(19) United States
(12) Patent Application Publication
(10) Pub. No.: US 2026/0317324 A1
(43) Pub. Date: Oct. 15, 2026

(54) BIFUNCTIONAL COMPOUNDS FOR DEGRADING BRAF VIA UBIQUITIN PROTEOSOME PATHWAY

(71) Applicant: Nurix Therapeutics, Inc., San Francisco, CA (US)

(72) Inventors: Ge PENG, San Francisco, CA (US); Jeffrey WU, San Francisco, CA (US)

(21) Appl. No.: 19/480,772

(22) PCT Filed: Apr. 26, 2024
(86) PCT No.: PCT/US2024/026679

§ 371 Date: Oct. 31, 2025

Related U.S. Application Data
(60) Provisional application No. 63/462,918, filed on Apr. 28, 2023.

(51) Int. Cl. C07D 471/10; A61K 31/4545; A61K 31/496; A61K 31/506; A61K 31/513; A61K 31/551; C07D 401/14; C07D 417/14

(52) U.S. Cl. CPC C07D 471/10; A61K 31/4545; A61K 31/496; A61K 31/506; A61K 31/513; A61K 31/551; C07D 401/14; C07D 417/14

(57) ABSTRACT

This disclosure relates to compounds of formula (I) useful for ex vivo, in vitro, or in vivo degradation of BRAF via a ubiquitin proteolytic pathway. This disclosure also provides pharmaceutically acceptable compositions comprising said compounds, and methods of using the compositions in the treatment of various diseases, conditions, and/or disorders. (I)

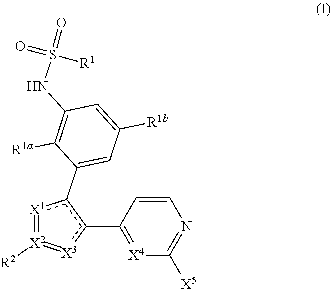

### CROSS REFERENCE

This patent application claims the benefit of U.S. Provisional Patent Application No. 63/462,918, filed Apr. 28, 2023, the contents of which are incorporated herein by reference in their entirety for all purposes.

### FIELD

This disclosure provides novel bifunctional compounds for proteolytically degrading targeted serine/threonine-protein kinase B-Raf (BRAF) and methods for treating diseases modulated by BRAF.

### BACKGROUND

Serine/threonine-protein kinase B-Raf (BRAF) participates in cellular signalling related to cell growth. BRAF provides a promising target for therapies for treating several inflammatory, autoimmune, and proliferative diseases and disorders.

### SUMMARY

Provided herein are compounds capable of binding, inhibiting, and/or degrading BRAF. The compounds are useful for the treatment or prevention of inflammatory, autoimmune, and proliferative diseases and disorders in a subject in need thereof. In certain embodiments, the compounds described herein include a BRAF hook bound to a linker further bound to a ubiquitin ligase harness.

In one aspect, provided herein are compounds according to Formula (I):

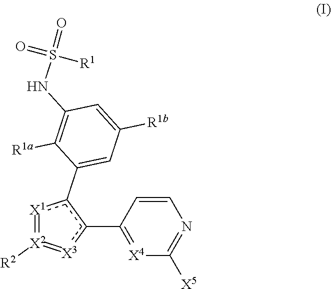

wherein
R¹ is alkyl, aryl, amino, alkylamino, dialkylamino, cycloalkyl, or heterocycloalkyl, each unsubstituted or substituted with one or more halogen or hydroxyl;
R^{1a} is halogen;
R^{1b} is hydrogen, haloalkyl, or halogen; R² is hydrogen, alkyl, cycloalkyl, haloalkyl, or —[Ar¹—Z¹]ₙ—Z²—Cy¹—Z³-L-UBM;
X¹ is carbon or nitrogen;
X² is carbon or nitrogen;
X³ is sulfur, nitrogen, oxygen, or carbon;
the bonds among X¹, X², and X³ comprise single and double bonds, and the ring containing X¹, X², and X³ is aromatic;
X⁴ is carbon or nitrogen;
X⁵ is hydrogen or —NR³R⁴;
R³ is hydrogen or alkyl where alkyl is unsubstituted or substituted with hydroxyl;
R⁴ is hydrogen, alkyl, cycloalkyl, or —[Ar¹—Z¹]ₙ—Z²—Cy¹—Z³-L-UBM wherein
   Ar¹ is arylene or heteroarylene, each unsubstituted or substituted with one or more alkyl or one or more halogen;
   Z¹ is a bond or —CH₂—;
   n is zero or one;
   Z² is absent, —C(O)—, —O—, C₁₋₁₀ alkylene, C₁₋₁₀ alkylene-C(Me)(Me)-, three- to six-membered cycloalkylene, three- to six-membered cycloalkylene-C₁₋₁₀ alkylene, heterocycloalkylene, or —C(Me)(Me)-;
   Cy¹ is absent or heterocycloalkylene, unsubstituted or substituted with one or more alkyl or one or more halogen;
   Z³ is absent, C₁₋₁₀ alkylene, or —C(O)—;
   L is a linker according to -L¹-L²-L³-L⁴- or -L⁴-L³-L²-L¹-, wherein
   L¹- is absent, —N(R¹⁰)—, —C(R¹¹)₂—, —C(O)—, —C₁₋₈ alkylene-, —C₂₋₈ alkynylene-, —C₆₋₁₀ aryl-, —C₄₋₁₀ heteroaryl-, -Q¹-, or -Q²-;
      each -L²-, -L³-, and -L⁴- is independently, absent, —N(R¹⁰)—, —C(R¹¹)₂—, —C(O)—, —O—, —(CH₂CH₂—O)₁₋₈—, —C₁₋₈ alkylene-, —C₂₋₈ alkynylene-, —C₆₋₁₀ aryl-, —C₄₋₁₀ heteroaryl-, -Q¹-, -Q²-, or -Q³-;
      each R¹⁰ is independently, hydrogen or methyl;
      each R¹¹ is, independently, hydrogen, methyl, aryl, or heteroaryl;
      each -Q¹- is a three- to seven-membered heterocycloalkylene comprising at least one nitrogen and is unsubstituted or substituted with one or more methyl, hydroxyl, or halogen;
      each -Q²- is a five- to thirteen-membered bicyclic heterocycloalkylene comprising at least one nitrogen, wherein the five- to thirteen-membered bicyclic heterocycloalkylene is optionally a spiro bicyclic heterocycloalkylene ring;
      each -Q³- is a three- to six-membered cycloalkylene; UBM is a ubiquitin ligase binding moiety;
wherein at least one of R² or R⁴ is —[Ar¹—Z¹]ₙ—Z²-Cy¹—Z³-L-UBM; or
a stereoisomer and/or pharmaceutical salt thereof.

In one aspect, provided herein are compounds according to Formula (I):

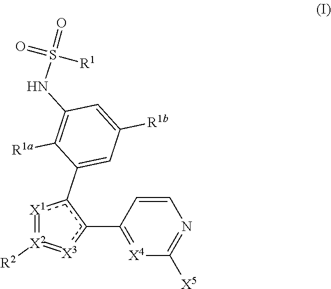

wherein
R¹ is alkyl, aryl, amino, alkylamino, dialkylamino, cycloalkyl, or heterocycloalkyl, each unsubstituted or substituted with a halogen or hydroxyl;
R^{1a} is halogen;
R^{1b} is hydrogen or halogen;
R² is hydrogen, alkyl, cycloalkyl, haloalkyl, or —[Ar¹—Z¹]ₙ—Z²-Cy¹—Z³-L-UBM;
X¹ is carbon or nitrogen;
X² is carbon or nitrogen;
X³ is sulfur, nitrogen, oxygen, or carbon;
the bonds among X¹, X², and X³ comprise single and double bonds, and the ring containing X¹, X², and X³ is aromatic;
X⁴ is carbon or nitrogen;
X⁵ is hydrogen or —NR³R⁴;
R³ is hydrogen or alkyl where alkyl is unsubstituted or substituted with hydroxyl;
R⁴ is hydrogen, alkyl, cycloalkyl, or —[Ar¹—Z¹]ₙ—Z²-Cy¹—Z³-L-UBM wherein
   Ar¹ is arylene or heteroarylene, each unsubstituted or substituted with one or more alkyl or one or more halogen;
   Z¹ is a bond or —CH₂—;
   n is zero or one.
   Z² is absent, —C(O)—, —O—, C₁₋₁₀ alkylene, C₁₋₁₀ alkylene-C(Me)(Me)-, three- to six-membered cycloalkylene, three- to six-membered cycloalkylene-C₁₋₁₀alkylene, heterocycloalkylene, or —C(Me)(Me)-;
   Cy¹ is absent or heterocycloalkylene, unsubstituted or substituted with alkyl;
   Z³ is absent, C₁₋₁₀ alkylene, or —C(O)—;
   L is a linker according to -L¹-L²-L³-L⁴- or -L⁴-L³-L²-L¹-, wherein
      -L¹- is absent, —N(R¹⁰)—, —C(R¹¹)₂—, —C(O)—, —C₁₋₈ alkylene-, —C₂₋₈ alkynylene-, —C₆₋₁₀ aryl-, —C₄₋₁₀ heteroaryl-, -Q¹-, or -Q²-;
      each -L²-, -L³-, and -L⁴- is independently, absent, —N(R¹⁰)—, —C(R¹¹)₂—, —C(O)—, —O—, —(CH₂CH₂—O)₁₋₈—, —C₁₋₈ alkylene-, —C₂₋₈ alkynylene-, —C₆₋₁₀ aryl-, —C₄₋₁₀ heteroaryl-, -Q¹-, -Q²-, or -Q³-;
      each R¹⁰ is independently, hydrogen or methyl;
      each R¹¹ is, independently, hydrogen, methyl, aryl, or heteroaryl;
      each -Q¹- is a three- to seven-membered heterocycloalkylene comprising at least one nitrogen and is unsubstituted or substituted with methyl, hydroxyl, or halogen;
      each -Q²- is a five- to thirteen-membered bicyclic heterocycloalkylene comprising at least one nitrogen, wherein the five- to thirteen-membered bicyclic heterocycloalkylene is optionally a spiro bicyclic heterocycloalkylene ring;
      each -Q³- is a three- to six-membered cycloalkylene; UBM is a ubiquitin ligase binding moiety;
         wherein at least one of R² or R⁴ is —[Ar¹—Z¹]n-Z²-Cy¹—Z³-L-UBM; or a stereoisomer and/or pharmaceutical salt thereof.

The compounds of Formula (I) comprise a BRAF hook. The BRAF hook is a moiety capable of binding BRAF in vitro, in vivo, and/or in a cell. Useful BRAF hooks are described herein. The ubiquitin ligase harness is a moiety capable of harnessing a ubiquitin ligase in vitro, in vivo, and/or in a cell. In certain embodiments, the ubiquitin ligase is an E3 ligase. Useful ubiquitin ligase harnesses are described herein. The compounds of Formula (I) further comprise a linker. The linker is any moiety capable of covalently binding the harness and the hook while permitting each to bind or harness its target. By harnessing a ubiquitin ligase and binding BRAF, the compounds of Formula (I) are capable of targeting BRAF for degradation under the appropriate conditions, for instance in a cell. Degrading BRAF provides a mechanism useful for treating inflammatory, autoimmune, and proliferative diseases and disorders in subjects in need thereof.

In another aspect, provided herein are compounds of Formula (II):

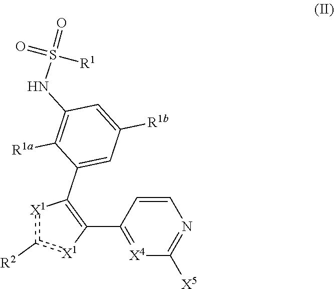

wherein
R¹ is alkyl, aryl, amino, alkylamino, dialkylamino, cycloalkyl, or heterocycloalkyl, each unsubstituted or substituted with a halogen or hydroxyl;
R^{1a} is halogen;
R^{1b} is hydrogen or halogen;
R² is —[Ar¹—Z¹]ₙ—Z²-Cy¹—Z³-L-UBM wherein
   Ar¹ is arylene or heteroarylene, each unsubstituted or substituted with one or more alkyl or one or more halogen;
   Z¹ is a bond or —CH₂—;
   n is zero or one;
   Z² is absent, —C(O)—, —O—, C₁₋₁₀ alkylene, C₁₋₁₀ alkylene-C(Me)(Me)-, three- to six-membered cycloalkylene, three- to six-membered cycloalkylene-C₁₋₁₀alkylene, heterocycloalkylene, or —C(Me)(Me)-;
   Cy¹ is absent or heterocycloalkylene, unsubstituted or substituted with alkyl;
   Z³ is absent, C₁₋₁₀ alkylene, or —C(O)—;
   L is a linker according to -L¹-L²-L³-L⁴- or -L⁴-L³-L²-L¹-, wherein
      -L¹- is absent, —N(R¹⁰)—, —C(R¹¹)₂—, —C(O)—, —C₁₋₈ alkylene-, —C₂₋₈ alkynylene-, —C₆₋₁₀ aryl-, —C₄₋₁₀ heteroaryl-, -Q¹-, or -Q²-;
      each -L²-, -L³-, and -L⁴- is independently, absent, —N(R¹⁰)—, —C(R¹¹)₂—, —C(O)—, —O—, —(CH₂CH₂—O)₁₋₈—, —C₁₋₈ alkylene-, —C₂₋₈ alkynylene-, —C₆₋₁₀ aryl-, —C₄₋₁₀ heteroaryl-, -Q¹-, -Q²-, or -Q³-;
      each R¹⁰ is independently, hydrogen or methyl;
      each R¹¹ is, independently, hydrogen, methyl, aryl, or heteroaryl;
      each -Q¹- is a three- to seven-membered heterocycloalkylene comprising at least one nitrogen and is unsubstituted or substituted with methyl, hydroxyl, or halogen;
      each -Q²- is a five- to thirteen-membered bicyclic heterocycloalkylene comprising at least one nitrogen, wherein the five- to thirteen-membered bicyclic heterocycloalkylene is optionally a spiro bicyclic heterocycloalkylene ring;
      each -Q³- is a three- to six-membered cycloalkylene;
   UBM is a ubiquitin ligase binding moiety;
X¹ is nitrogen or oxygen;
X³ is sulfur when X¹ is nitrogen; oxygen when X¹ is nitrogen; or nitrogen when X¹ is oxygen;
the bonds among X¹ and X³ comprise single and double bonds, and the ring containing
X¹ and X³ is aromatic;
X⁴ is carbon or nitrogen;
X⁵ is hydrogen or —NR³R⁴;
R³ is hydrogen or alkyl where alkyl is unsubstituted or substituted with hydroxyl;
R⁴ is hydrogen, alkyl, or cycloalkyl; or
   a stereoisomer and/or pharmaceutical salt thereof.

As described in more detail elsewhere herein, one side of the molecules of Formula I or II is a ubiquitin ligase harness. The other side of the molecules is a BRAF hook. The middle portion of the molecules is a linker.

In another aspect, provided herein are pharmaceutical compositions. The pharmaceutical compositions comprise the compounds of Formula (I) and/or (II) along with one or more pharmaceutically acceptable carriers, diluents, and/or excipients.

In another aspect, provided herein are methods of treating a disease or disorder in a subject in need thereof comprising the step of administering to the subject a therapeutically effective amount of the compound of Formula (I) and/or (II), or pharmaceutical compositions thereof to the subject. In certain embodiments, provided herein are the compounds and compositions of Formula (I) and/or (II) for use in therapy. In certain embodiments, provided herein are the compounds and compositions of Formula (I) and/or (II) for use in the treatment or prevention of inflammatory, autoimmune, or proliferative diseases and disorders. In certain embodiments, provided herein are the uses of the compounds and compositions of Formula (I) and/or (II) for the manufacture of medicaments. In certain embodiments, provided herein are the uses of the compounds and compositions of Formula (I) and/or (II) for the manufacture of medicaments for the treatment or prevention of inflammatory, autoimmune, or proliferative diseases and disorders. In certain embodiments, the disease or disorder is an autoimmune disease or disorder. In certain embodiments, the disease or disorder is a proliferative disease or disorder, for instance, a T-cell lymphoma. In certain embodiments, the disease or disorder is cancer. In certain embodiments, the compounds or compositions of Formula (I) and/or (II) are for use in the treatment of cancer.

### DETAILED DESCRIPTION

### Definitions

For purposes herein, the chemical elements are identified in accordance with the Periodic Table of the Elements, CAS version, Handbook of Chemistry and Physics, 75th Ed. Additionally, general principles of organic chemistry are described in “Organic Chemistry,” Thomas Sorrell, University Science Books, Sausalito: 1999, and “March's Advanced Organic Chemistry,” 5th Ed., Ed.: Smith, M. B. and March, J., John Wiley & Sons, New York: 2001, the entire contents of which are hereby incorporated herein by reference.

As described herein, “protecting group” refers to a moiety or functionality that is introduced into a molecule by chemical modification of a functional group in order to obtain chemoselectivity in a subsequent chemical reaction. Standard protecting groups are provided in Wuts and Greene: “Greene's Protective Groups in Organic Synthesis,” 4th Ed, Wuts, P. G. M. and Greene, T. W., Wiley-Interscience, New York: 2006.

As described herein, compounds herein optionally may be substituted with one or more substituents, such as those illustrated generally herein, or as exemplified by particular classes, subclasses, and/or species of this description.

As used herein, the term “hydroxyl” or “hydroxy” refers to an —OH moiety.

As used herein, the term “aliphatic” encompasses the terms alkyl, alkenyl, and alkynyl, each of which are optionally substituted as set forth below.

As used herein, an “alkyl” group refers to a saturated aliphatic hydrocarbon group containing one to twelve (e.g., one to eight, one to six, or one to four) carbon atoms. An alkyl group can be straight or branched. Examples of alkyl groups include, but are not limited to, methyl, ethyl, propyl, isopropyl, butyl, isobutyl, sec-butyl, tert-butyl, n-pentyl, n-heptyl, or 2-ethylhexyl. An alkyl group can be substituted (i.e., optionally substituted) with one or more substituents such as halo, phospho, cycloaliphatic (e.g., cycloalkyl or cycloalkenyl), heterocycloaliphatic (e.g., heterocycloalkyl or heterocycloalkenyl), aryl, heteroaryl, alkoxy, aroyl, heteroaroyl, acyl (e.g., (aliphatic)carbonyl, (cycloaliphatic)carbonyl, or (heterocycloaliphatic)carbonyl), nitro, cyano, amido (e.g., (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, alkylaminocarbonyl, cycloalkylaminocarbonyl, heterocycloalkylaminocarbonyl, arylaminocarbonyl, or heteroarylaminocarbonyl), amino (e.g., aliphaticamino, cycloaliphaticamino, or heterocycloaliphaticamino), sulfonyl (e.g., aliphatic-SO₂—, cycloaliphatic-SO₂—, or aryl-SO₂—), sulfinyl, sulfanyl, sulfoxy, urea, thiourea, sulfamoyl, sulfamide, oxo, carboxy, carbamoyl, cycloaliphaticoxy, heterocycloaliphaticoxy, aryloxy, heteroaryloxy, aralkyloxy, heteroarylalkoxy, alkoxycarbonyl, alkylcarbonyloxy, or hydroxy. Without limitation, some examples of substituted alkyls include carboxyalkyl (such as HOOC-alkyl, alkoxycarbonylalkyl, and alkylcarbonyloxyalkyl), cyanoalkyl, hydroxyalkyl, alkoxyalkyl, acylalkyl, aralkyl, (alkoxyaryl)alkyl, (sulfonylamino)alkyl (such as (alkyl-SO₂-amino)alkyl), aminoalkyl, amidoalkyl, (cycloaliphatic)alkyl, or haloalkyl.

As used herein, an “alkenyl” group refers to an aliphatic carbon group that contains two to eight (e.g., two to four or two to six) carbon atoms and at least one double bond. Like an alkyl group, an alkenyl group can be straight or branched. Examples of an alkenyl group include, but are not limited to, allyl, 1- or 2-isopropenyl, 2-butenyl, and 2-hexenyl. An alkenyl group can be optionally substituted with one or more substituents such as halo, phospho, cycloaliphatic (e.g., cycloalkyl or cycloalkenyl), heterocycloaliphatic (e.g., heterocycloalkyl or heterocycloalkenyl), aryl, heteroaryl, alkoxy, aroyl, heteroaroyl, acyl (e.g., (aliphatic)carbonyl, (cycloaliphatic)carbonyl, or (heterocycloaliphatic)carbonyl), nitro, cyano, amido (e.g., (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, alkylaminocarbonyl, cycloalkylaminocarbonyl, heterocycloalkylaminocarbonyl, arylaminocarbonyl, or heteroarylaminocarbonyl), amino (e.g., aliphaticamino, cycloaliphaticamino, heterocycloaliphaticamino, or aliphaticsulfonylamino), sulfonyl (e.g., alkyl-SO₂—, cycloaliphatic-SO₂—, or aryl-SO₂—), sulfinyl, sulfanyl, sulfoxy, urea, thiourea, sulfamoyl, sulfamide, oxo, carboxy, carbamoyl, cycloaliphaticoxy, heterocycloaliphaticoxy, aryloxy, heteroaryloxy, aralkyloxy, heteroaralkoxy, alkoxycarbonyl, alkylcarbonyloxy, or hydroxy. Without limitation, some examples of substituted alkenyls include cyanoalkenyl, alkoxyalkenyl, acylalkenyl, hydroxyalkenyl, aralkenyl, (alkoxyaryl)alkenyl, (sulfonylamino)alkenyl (such as (alkyl-SO₂-amino)alkenyl), aminoalkenyl, amidoalkenyl, (cycloaliphatic)alkenyl, or haloalkenyl.

As used herein, an “alkynyl” group refers to an aliphatic carbon group that contains two to eight (e.g., two to four or two to six) carbon atoms and has at least one triple bond. An alkynyl group can be straight or branched. Examples of an alkynyl group include, but are not limited to, propargyl and butynyl. An alkynyl group can be optionally substituted with one or more substituents such as aroyl, heteroaroyl, alkoxy, cycloalkyloxy, heterocycloalkyloxy, aryloxy, heteroaryloxy, aralkyloxy, nitro, carboxy, cyano, halo, hydroxy, sulfo, mercapto, sulfanyl (e.g., aliphaticsulfanyl or cycloaliphaticsulfanyl), sulfinyl (e.g., aliphaticsulfinyl or cycloaliphaticsulfinyl), sulfonyl (e.g., aliphatic-SO₂—, aliphaticamino-SO₂—, or cycloaliphatic-SO₂—), amido (e.g., aminocarbonyl, alkylaminocarbonyl, alkylcarbonylamino, cycloalkylaminocarbonyl, heterocycloalkylaminocarbonyl, cycloalkylcarbonylamino, arylaminocarbonyl, arylcarbonylamino, aralkylcarbonylamino, (heterocycloalkyl)carbonylamino, (cycloalkylalkyl)carbonylamino, heteroaralkylcarbonylamino, heteroarylcarbonylamino, or heteroarylaminocarbonyl), urea, thiourea, sulfamoyl, sulfamide, alkoxycarbonyl, alkylcarbonyloxy, cycloaliphatic, heterocycloaliphatic, aryl, heteroaryl, acyl (e.g., (cycloaliphatic)carbonyl or (heterocycloaliphatic)carbonyl), amino (e.g., aliphaticamino), sulfoxy, oxo, carboxy, carbamoyl, (cycloaliphatic)oxy, (heterocycloaliphatic)oxy, or (heteroaryl)alkoxy.

As used herein, an “amido” group encompasses both “aminocarbonyl” and “carbonylamino.” These terms when used alone or in connection with another group refer to an amido group such as —N(R^{X})—C(O)—R^{Y} or —C(O)—N(R^{X})₂ when used terminally, and —C(O)—N(R^{X})— or —N(R^{X})—C(O)— when used internally, wherein R^{X} and R^{Y} can be aliphatic, cycloaliphatic, aryl, araliphatic, heterocycloaliphatic, heteroaryl, or heteroaraliphatic. Examples of amido groups include alkylamido (such as alkylcarbonylamino or alkylaminocarbonyl), (heterocycloaliphatic)amido, (heteroaralkyl)amido, (heteroaryl)amido, (heterocycloalkyl)alkylamido, arylamido, aralkylamido, (cycloalkyl)alkylamido, or cycloalkylamido.

As used herein, an “amino” group refers to —NR^{X}R^{Y} wherein each of R^{X} and R^{Y} is independently hydrogen (H or —H), aliphatic, cycloaliphatic, (cycloaliphatic)aliphatic, aryl, araliphatic, heterocycloaliphatic, (heterocycloaliphatic)aliphatic, heteroaryl, carboxy, sulfanyl, sulfinyl, sulfonyl, (aliphatic)carbonyl, (cycloaliphatic)carbonyl, ((cycloaliphatic)aliphatic)carbonyl, arylcarbonyl, (araliphatic)carbonyl, (heterocycloaliphatic)carbonyl, ((heterocycloaliphatic)aliphatic)carbonyl, (heteroaryl)carbonyl, or (heteroaraliphatic)carbonyl, each of which being defined elsewhere herein and being optionally substituted. Examples of amino groups include alkylamino, dialkylamino, or arylamino. When the term “amino” is not the terminal group (e.g., alkylcarbonylamino), it is represented by —NR^{X}—, where R^{X} has the same meaning as defined above.

As used herein, an “aryl” group used alone or as part of a larger moiety as in “aralkyl,” “aralkoxy,” or “aryloxyalkyl” refers to monocyclic (e.g., phenyl); bicyclic (e.g., indenyl, naphthalenyl, tetrahydronaphthyl, or tetrahydroindenyl); and tricyclic (e.g., fluorenyl tetrahydrofluorenyl, tetrahydroanthracenyl, or anthracenyl) ring systems in which the monocyclic ring system is aromatic or at least one of the rings in a bicyclic or tricyclic ring system is aromatic. The bicyclic and tricyclic groups include benzofused (e.g., 2- or 3-membered, or bi- or tricyclic carbocyclic) rings. For example, a benzofused group includes phenyl fused with two or more C₄₋₈ carbocyclic moieties. An aryl is optionally substituted with one or more substituents including aliphatic (e.g., alkyl, alkenyl, or alkynyl); cycloaliphatic; (cycloaliphatic)aliphatic; heterocycloaliphatic; (heterocycloaliphatic)aliphatic; aryl; heteroaryl; alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; oxo (i.e., on a non-aromatic carbon within a carbocyclic ring of a benzofused bicyclic or tricyclic aryl); nitro; carboxy; amido; acyl (e.g., (aliphatic)carbonyl; (cycloaliphatic)carbonyl; ((cycloaliphatic)aliphatic)carbonyl; (araliphatic)carbonyl; (heterocycloaliphatic)carbonyl; ((heterocycloaliphatic)aliphatic)carbonyl; or (heteroaraliphatic)carbonyl); sulfonyl (e.g., aliphatic-SO₂— or amino-SO₂—); sulfinyl (e.g., aliphatic-S(O)— or cycloaliphatic-S(O)—); sulfanyl (e.g., aliphatic-S—); cyano; halo; hydroxy; mercapto; sulfoxy; urea; thiourea; sulfamoyl; sulfamide; or carbamoyl. Alternatively, an aryl can be unsubstituted. As used herein, an “arylene” is a divalent version of “aryl.”

Non-limiting examples of substituted aryls include haloaryl (e.g., mono-, di-(such as p,m-dihaloaryl), and (tri-halo)aryl); (carboxy)aryl (e.g., (alkoxycarbonyl)aryl, ((aralkyl)carbonyloxy)aryl, and (alkoxycarbonyl)aryl); (amido)aryl (e.g., (aminocarbonyl)aryl, (((alkylamino)alkyl)aminocarbonyl)aryl, (alkylcarbonyl)aminoaryl, (arylaminocarbonyl)aryl, and (((heteroaryl)amino)carbonyl)aryl); aminoaryl (e.g., ((alkylsulfonyl)amino)aryl or ((dialkyl)amino)aryl); (cyanoalkyl)aryl; (alkoxy)aryl; (sulfamoyl)aryl (e.g., (aminosulfonyl)aryl); (alkylsulfonyl)aryl; (cyano)aryl; (hydroxyalkyl)aryl; ((alkoxy)alkyl)aryl; (hydroxy)aryl, ((carboxy)alkyl)aryl; (((dialkyl)amino)alkyl)aryl; (nitroalkyl)aryl; (((alkylsulfonyl)amino)alkyl)aryl; ((heterocycloaliphatic)carbonyl)aryl; ((alkylsulfonyl)alkyl)aryl; (cyanoalkyl)aryl; (hydroxyalkyl)aryl; (alkylcarbonyl)aryl; alkylaryl; (trihaloalkyl)aryl; p-amino-m-alkoxycarbonylaryl; p-amino-m-cyanoaryl; p-halo-m-aminoaryl; or (m-(heterocycloaliphatic)-o-(alkyl))aryl.

As used herein, an “araliphatic” such as an “aralkyl” group refers to an aliphatic group (e.g., a C₁₋₄ alkyl group) that is substituted with an aryl group. “Aliphatic,” “alkyl,” and “aryl” are defined elsewhere herein. An example of an araliphatic such as an aralkyl group is benzyl.

As used herein, an “aralkyl” group refers to an alkyl group (e.g., a C₁₋₄ alkyl group) that is substituted with an aryl group. Both “alkyl” and “aryl” have been defined above. An example of an aralkyl group is benzyl. An aralkyl is optionally substituted with one or more substituents such as aliphatic (e.g., alkyl, alkenyl, or alkynyl, including carboxyalkyl, hydroxyalkyl, or haloalkyl such as trifluoromethyl), cycloaliphatic (e.g., cycloalkyl or cycloalkenyl), (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, amido (e.g., aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, or heteroaralkylcarbonylamino), cyano, halo, hydroxy, acyl, mercapto, alkylsulfanyl, sulfoxy, urea, thiourea, sulfamoyl, sulfamide, oxo, or carbamoyl.

As used herein, a “bicyclic ring system” includes 6- to 12-membered (e.g., 8- to 12- or 9-, 10-, or 11-membered) structures that form two rings, wherein the two rings have at least one atom in common (e.g., two atoms in common). Bicyclic ring systems include bicycloaliphatics (e.g., bicycloalkyl or bicycloalkenyl), bicycloheteroaliphatics, bicyclic aryls, and bicyclic heteroaryls.

As used herein, a “cycloaliphatic” group encompasses a “cycloalkyl” group and a “cycloalkenyl” group, each of which are optionally substituted as set forth below.

As used herein, a “cycloalkyl” group refers to a saturated carbocyclic mono- or bicyclic (fused, bridged, or spiro) ring of three to ten (e.g., five to ten) carbon atoms. Examples of cycloalkyl groups include cyclopropyl, cyclobutyl, cyclopentyl, cyclohexyl, cycloheptyl, adamantyl, norbornyl, cubyl, octahydro-indenyl, decahydro-naphthyl, bicyclo[3.2.1]octyl, bicyclo[2.2.2]octyl, bicyclo[3.3.1]nonyl, bicyclo[3.3.2]decyl, bicyclo[2.2.2]octyl, adamantyl, spiro[3.3]heptanyl, or ((aminocarbonyl)cycloalkyl)cycloalkyl.

A “cycloalkenyl” group, as used herein, refers to a non-aromatic carbocyclic ring of three to ten (e.g., four to eight) carbon atoms having one or more double bonds. Examples of cycloalkenyl groups include cyclopentenyl, 1,4-cyclohexa-di-enyl, cycloheptenyl, cyclooctenyl, hexahydro-indenyl, octahydro-naphthyl, cyclohexenyl, bicyclo[2.2.2]octenyl, or bicyclo[3.3.1]nonenyl.

A “cycloalkyl” or “cycloalkenyl” group can be optionally substituted with one or more substituents such as phospho, aliphatic (e.g., alkyl, alkenyl, or alkynyl), cycloaliphatic, (cycloaliphatic)aliphatic, heterocycloaliphatic, (heterocycloaliphatic)aliphatic, aryl, heteroaryl, alkoxy, (cycloaliphatic)oxy, (heterocycloaliphatic)oxy, aryloxy, heteroaryloxy, (araliphatic)oxy, (heteroaraliphatic)oxy, aroyl, heteroaroyl, amino, amido (e.g., (aliphatic)carbonylamino, (cycloaliphatic)carbonylamino, ((cycloaliphatic)aliphatic)carbonylamino, (aryl)carbonylamino, (araliphatic)carbonylamino, (heterocycloaliphatic)carbonylamino, ((heterocycloaliphatic)aliphatic)carbonylamino, (heteroaryl)carbonylamino, or (heteroaraliphatic)carbonylamino), nitro, carboxy (e.g., HOOC—, alkoxycarbonyl, or alkylcarbonyloxy), acyl (e.g., (cycloaliphatic)carbonyl, ((cycloaliphatic)aliphatic)carbonyl, (araliphatic)carbonyl, (heterocycloaliphatic)carbonyl, ((heterocycloaliphatic)aliphatic)carbonyl, or (heteroaraliphatic)carbonyl, cyano, halo, hydroxy, mercapto, sulfonyl (e.g., alkyl-SO₂— and aryl-SO₂—), sulfinyl (e.g., alkyl-S(O)—), sulfanyl (e.g., alkyl-S—), sulfoxy, urea, thiourea, sulfamoyl, sulfamide, oxo, or carbamoyl.

As used herein, the term “heterocycloaliphatic” encompasses heterocycloalkyl groups and heterocycloalkenyl groups, each of which being optionally substituted as set forth below.

As used herein, a “heterocycloalkyl” or divalent “heterocycloalkylene” group refers to a 3- to 15-membered mono- or bicylic (e.g., 5- to 10-membered mono- or bicyclic) saturated ring structure (e.g., fused, bridged, or spiro), in which one or more of the ring atoms is a heteroatom (e.g., nitrogen (N), oxygen (O), sulfur (S), or combinations thereof). Non-limiting examples of a heterocycloalkyl (or a divalent heterocycloalkylene) group include piperidyl or piperidinyl, piperazyl or piperazinyl, tetrahydropyranyl, tetrahydrofuryl or tetrahydrofuranyl, 1,4-dioxolanyl, 1,4-dithianyl, 1,3-dioxolanyl, 1,3-diazepanyl, 1,4-diazepanyl, oxazolidyl or oxazolidinyl, isoxazolidyl or isoxazolidinyl, morpholinyl, thiomorpholinyl, octahydrobenzofuryl or octahydrobenzofuranyl, octahydrochromenyl, octahydrothiochromenyl, octahydroindolyl or octahydroindolinyl, octahydropyrindinyl or octahydro-1H-cyclopenta[x]pyridine where x is b or c, decahydroquinolinyl, octahydrobenzo[b]thiopheneyl, 2-oxa-bicyclo[2.2.2]octyl, 1-aza-bicyclo[2.2.2]octyl, 3-aza-bicyclo[3.2.1]octyl, 3,8-diazabicyclo[3.2.1]octyl, decahydro-2,7-naphthyridine, 2,8-diazaspiro[4.5]decane, 2-azaspiro[3.3]heptane, 2-azaspiro[3.5]nonane, 3-azaspiro[5.5]undecane, 2,7-diazaspiro[3.5]nonane, octahydropyrrolo[3,4-c]pyrrole, octahydro-1H-pyrrolo[3,4-b]pyridine, 2,5-diazabicyclo[2.2.1]heptane, 1-oxa-8-azaspiro[4.5]decane, and 2,6-dioxa-tricyclo[3.3.1.0^{3,7}]nonyl. A monocyclic heterocycloalkyl group can be fused with a phenyl moiety to form, for example, tetrahydroisoquinoline, that could be categorized as a heteroaryl as defined elsewhere herein.

A “heterocycloalkenyl” group, as used herein, refers to a mono- or bicylic (e.g., 5- to 10-membered mono- or bicyclic) non-aromatic ring structure having one or more double bonds, and wherein one or more of the ring atoms is a heteroatom (e.g., nitrogen (N), oxygen (O), or sulfur (S)). Monocyclic and bicyclic heterocycloalkenyls are numbered according to standard chemical nomenclature.

A heterocycloalkyl or heterocycloalkenyl group can be optionally substituted with one or more substituents such as phospho, aliphatic (e.g., alkyl, alkenyl, or alkynyl), cycloaliphatic, (cycloaliphatic)aliphatic, heterocycloaliphatic, (heterocycloaliphatic)aliphatic, aryl, heteroaryl, alkoxy, (cycloaliphatic)oxy, (heterocycloaliphatic)oxy, aryloxy, heteroaryloxy, (araliphatic)oxy, (heteroaraliphatic)oxy, aroyl, heteroaroyl, amino, amido (e.g., (aliphatic)carbonylamino, (cycloaliphatic)carbonylamino, ((cycloaliphatic)aliphatic)carbonylamino, (aryl)carbonylamino, (araliphatic)carbonylamino, (heterocycloaliphatic)carbonylamino, ((heterocycloaliphatic)aliphatic)carbonylamino, (heteroaryl)carbonylamino, or (heteroaraliphatic)carbonylamino, nitro, carboxy (e.g., HOOC—, alkoxycarbonyl, or alkylcarbonyloxy), acyl (e.g., (cycloaliphatic)carbonyl, ((cycloaliphatic)aliphatic)carbonyl, (araliphatic)carbonyl, (heterocycloaliphatic)carbonyl, ((heterocycloaliphatic)aliphatic)carbonyl, or (heteroaraliphatic)carbonyl), nitro, cyano, halo, hydroxy, mercapto, sulfonyl (e.g., alkylsulfonyl or arylsulfonyl), sulfinyl (e.g., alkylsulfinyl), sulfanyl (e.g., alkylsulfanyl), sulfoxy, urea, thiourea, sulfamoyl, sulfamide, oxo, or carbamoyl.

A “heteroaryl” group, as used herein, refers to a monocyclic, bicyclic, or tricyclic ring system having four to fifteen ring atoms wherein one or more of the ring atoms is a heteroatom (e.g., N, O, S, or combinations thereof) and in which the monocyclic ring system is aromatic or at least one of the rings in the bicyclic or tricyclic ring systems is aromatic. A heteroaryl group includes a benzofused ring system having two to three rings. For example, a benzofused group includes one or two 4- to 8-membered heterocycloaliphatic moieties (e.g., indolizyl, indolyl, isoindolyl, isoindolinyl-1,3-dione, isoindolinyl-1-one, 3H-indolyl, indolinyl, benzo[b]furyl, benzo[b]thiopheneyl, quinolinyl, or isoquinolinyl). Some examples of heteroaryl are pyridyl, 1H-indazolyl, furyl or furanyl, pyrrolyl, thienyl, thiazolyl, oxazolyl, imidazolyl, tetrazolyl, benzofuryl or benzofuranyl, isoquinolinyl, 1-oxoisoquinolin-2(1H)-yl, benzthiazolyl, xanthene, thioxanthene, phenothiazine, dihydroindole, benzo[1,3]dioxole, benzo[b]furyl, benzo[b]thiopheneyl, indazolyl, benzimidazolyl, 3-methyl-2-oxo-2,3-dihydro-1H-benzo[d]imidazol-1-yl, benzthiazolyl, puryl or purinyl, cinnolyl, quinolyl, quinazolyl, phthalazyl, quinazolyl, quinoxalyl, isoquinolyl, 4H-quinolizyl, benzo-1,2,5-thiadiazolyl, or 1,8-naphthyridyl. Other examples of heteroaryls include 1,2,3,4-tetrahydroisoquinoline and 4,5,6,7-tetrahydropyrazolo[1,5-a]pyrazine. As used herein, “heteroarylene” is a divalent version of “heteroaryl.”

Without limitation, monocyclic heteroaryls include furyl, thiophene-yl, 2H-pyrrolyl, oxazolyl, thiazolyl, imidazolyl, pyrazolyl, isoxazolyl, isothiazolyl, 1,3,4-thiadiazolyl, pyridyl, pyridazyl, pyrimidyl, pyrazolyl, pyrazyl, or 1,3,5-triazyl. Monocyclic heteroaryls are numbered according to standard chemical nomenclature.

Without limitation, bicyclic heteroaryls include indolizyl, indolyl, isoindolyl, 3H-indolyl, indolinyl, benzo[b]furyl, benzo[b]thiopheneyl, quinolinyl, isoquinolinyl, indazolyl, benzimidazyl, benzthiazolyl, purinyl, 4H-quinolizyl, quinolyl, isoquinolyl, cinnolyl, phthalazyl, quinazolyl, quinoxalyl, 1,8-naphthyridyl, or pteridyl. Bicyclic heteroaryls are numbered according to standard chemical nomenclature.

A heteroaryl is optionally substituted with one or more substituents such as aliphatic (e.g., alkyl, alkenyl, or alkynyl); cycloaliphatic; (cycloaliphatic)aliphatic; heterocycloaliphatic; (heterocycloaliphatic)aliphatic; aryl; heteroaryl; alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; oxo (on a non-aromatic carbocyclic or heterocyclic ring of a bicyclic or tricyclic heteroaryl); carboxy; amido; acyl (e.g., (aliphatic)carbonyl; (cycloaliphatic)carbonyl; ((cycloaliphatic)aliphatic)carbonyl; (araliphatic)carbonyl; (heterocycloaliphatic)carbonyl; ((heterocycloaliphatic)aliphatic)carbonyl; or (heteroaraliphatic)carbonyl); sulfonyl (e.g., aliphaticsulfonyl or aminosulfonyl); sulfinyl (e.g., aliphaticsulfinyl); sulfanyl (e.g., aliphaticsulfanyl); nitro; cyano; halo; hydroxy; mercapto; sulfoxy; urea; thiourea; sulfamoyl; sulfamide; or carbamoyl. Alternatively, a heteroaryl can be unsubstituted.

Non-limiting examples of substituted heteroaryls include (halo)heteroaryl (e.g., mono- and di-(halo)heteroaryl); (carboxy)heteroaryl (e.g., (alkoxycarbonyl)heteroaryl); cyanoheteroaryl; aminoheteroaryl (e.g., ((alkylsulfonyl)amino)heteroaryl and ((dialkyl)amino)heteroaryl); (amido)heteroaryl (e.g., aminocarbonylheteroaryl, ((alkylcarbonyl)amino)heteroaryl, ((((alkyl)amino)alkyl)aminocarbonyl)heteroaryl, (((heteroaryl)amino)carbonyl)heteroaryl, ((heterocycloaliphatic)carbonyl)heteroaryl, and ((alkylcarbonyl)amino)heteroaryl); (cyanoalkyl)heteroaryl; (alkoxy)heteroaryl; (sulfamoyl)heteroaryl (e.g., (aminosulfonyl)heteroaryl); (sulfonyl)heteroaryl (e.g., (alkylsulfonyl)heteroaryl); (hydroxyalkyl)heteroaryl; (alkoxyalkyl)heteroaryl; (hydroxy)heteroaryl; ((carboxy)alkyl)heteroaryl; (((dialkyl)amino)alkyl)heteroaryl; (heterocycloaliphatic)heteroaryl; (cycloaliphatic)heteroaryl; (nitroalkyl)heteroaryl; (((alkylsulfonyl)amino)alkyl)heteroaryl; ((alkylsulfonyl)alkyl)heteroaryl; (cyanoalkyl)heteroaryl; (acyl)heteroaryl (e.g., (alkylcarbonyl)heteroaryl); (alkyl)heteroaryl; or (haloalkyl)heteroaryl (e.g., trihaloalkylheteroaryl).

As used herein, a “heteroaraliphatic” (such as a heteroaralkyl group) refers to an aliphatic group (e.g., a C₁₋₄ alkyl group) that is substituted with a heteroaryl group. “Aliphatic,” “alkyl,” and “heteroaryl” have been defined above.

As used herein, a “heteroaralkyl” group refers to an alkyl group (e.g., a C₁₋₄ alkyl group) that is substituted with a heteroaryl group. Both “alkyl” and “heteroaryl” have been defined above. A heteroaralkyl is optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, or carbamoyl.

As used herein, “cyclic moiety” and “cyclic group” refer to mono-, bi-, and tricyclic ring systems including cycloaliphatic, heterocycloaliphatic, aryl, or heteroaryl, each of which has been previously defined.

As used herein, a “bridged bicyclic ring system” refers to a bicyclic heterocycloalipahtic (or heterocycloaliphatic) ring system or bicyclic cycloaliphatic ring system in which the rings are bridged. Examples of bridged bicyclic ring systems include, but are not limited to, adamantanyl, norbornanyl, bicyclo[3.2.1]octyl, bicyclo[2.2.2]octyl, bicyclo[3.3.1]nonyl, bicyclo[3.3.2]decyl, 2-oxabicyclo[2.2.2]octyl, 1-azabicyclo[2.2.2]octyl, 3-azabicyclo[3.2.1]octyl, and 2,6-dioxa-tricyclo[3.3.1.0^{3,7}]nonyl. A bridged bicyclic ring system can be optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, or carbamoyl.

As used herein, an “acyl” group refers to a R^{X}—C(O)— (such as alkyl-C(O)—, also referred to as “alkylcarbonyl”) where R^{X} and “alkyl” have been defined previously. Acetyl and pivaloyl are examples of acyl groups.

As used herein, an “aroyl” or “heteroaroyl” refers to an aryl-C(O)— or a heteroaryl-C(O)—. The aryl and heteroaryl portion of the aroyl or heteroaroyl is optionally substituted as previously defined herein. For example, aroyl includes benzoyl.

As used herein, an “alkoxy” group refers to an alkyl-O— group where “alkyl” has been defined previously herein.

As used herein, a “carbamoyl” group refers to a group having the formula —O—CO—NR^{X}R^{Y} or —NR^{X}—CO—O—R^{Z}, wherein R^{X} and R^{Y} have been defined above and R^{Z} can be aliphatic, aryl, araliphatic, heterocycloaliphatic, heteroaryl, or heteroaraliphatic.

As used herein, a “carboxy” group refers to —COOH, when used as a terminal group; or —OC(O)—, or —C(O)O— when used as an internal group.

As used herein, an “ester” refers to —COOR^{X} when used as a terminal group; or —COOR^{X}— when used as an internal group, wherein R^{X} has been defined above. As used herein, an “alkoxycarbonyl,” which is encompassed by the term ester, used alone or in connection with another group refers to a group such as alkyl-O—C(O)— (i.e., a carbonate ester).

As used herein, a “formate” refers to —OC(O)H.

As used herein, an “acetate” refers to —OC(O)R^{X}, wherein R^{X} has been defined above. In one embodiment, acetate is —OC(O)Me

As used herein, a “haloaliphatic” group refers to an aliphatic group substituted with one to three halogen atoms. For instance, haloalkyl includes —CF₃.

As used herein, a “mercapto” or “sulfhydryl” group refers to —SH.

As used herein, a “sulfo” group refers to —SO₃H, or —SO₃R^{X} when used terminally or —S(O)₃— when used internally. In one embodiment, —SO₃H is a sulfonic acid. In one embodiment, R^{X}SO₃⁻ is a sulfonate.

As used herein, a “sulfamide” group refers to the formula —NR^{X}—S(O)₂—NR^{Y}R^{Z} when used terminally and —NR^{X}—S(O)₂—NR^{Y}— when used internally, wherein R^{X}, R^{Y}, and R^{Z} have been defined above.

As used herein, a “sulfamoyl” group refers to the formula —S(O)₂—NR^{Y}R^{Z} wherein R^{Y}, and R^{Z} have been defined above. In one embodiment, —O—S(O)₂—NR^{Y}R^{Z} is a sulfamate.

As used herein, a “sulfonamide” group refers to the formula R^{Z}-S(O)₂—NR^{X}R^{Y} or —NR^{X}—S(O)₂—R^{Z} when used terminally; or —S(O)₂—NR^{X}—, or —NR^{X}—S(O)₂—when used internally, wherein R^{X}, R^{Y}, and R^{Z} are defined above.

As used herein a “sulfide” group refers to —S—R^{X} when used terminally and —S-when used internally, wherein R^{X} has been defined above. Examples of sulfanyls include aliphatic-S—, cycloaliphatic-S—, aryl-S—, or the like.

As used herein a “sulfinyl” group refers to —S(O)—R^{X} when used terminally and —S(O)—when used internally, wherein R^{X} has been defined above. Examples of sulfinyl groups include aliphatic-S(O)—, aryl-S(O)—, (cycloaliphatic(aliphatic))-S(O)—, cycloalkyl-S(O)—, heterocycloaliphatic-S(O)—, heteroaryl-S(O)—, and/or the like.

As used herein, a “sulfonyl” group refers to —S(O)₂—R^{X} when used terminally and —S(O)₂—when used internally, wherein R^{X} has been defined above. Examples of sulfonyl groups include aliphatic-S(O)₂—, aryl-S(O)₂—, (cycloaliphatic(aliphatic))-S(O)₂—, cycloaliphatic-S(O)₂—, heterocycloaliphatic-S(O)₂—, heteroaryl-S(O)₂—, (cycloaliphatic(amido(aliphatic)))-S(O)₂—, and/or the like.

As used herein, a “sulfinate” group refers to —O—S(O)—R^{X}, or —S(O)—O-R^{X}, when used terminally and —O—S(O)— or —S(O)—O—when used internally, where R^{X} has been defined above.

As used herein, a “halogen” or “halo” group refers to fluorine (F), chlorine (C₁), bromine (Br), or iodine (I).

As used herein, an “alkoxyalkyl” refers to an alkyl group modified with an alkoxy group, such as alkyl-O-alkyl- or ethers, wherein alkyl has been defined above.

As used herein, a “carbonyl” or “oxo” refers to —C(O)— or ═O.

As used herein, the term “phospho” refers to phosphinates, phosphonates, phosphine oxides, phosphoramidates, phosphinic amides, and phosphonamidates. Examples of phosphinates, phosphonates, phosphine oxides, phosphoramidates, phosphinic amides, and phosphonamidates include —P(O)(R^{P})₂, (R^{P})₂P(O)OR^{P}, and R^{P}—PO(OR^{P})₂, wherein R^{P} is aliphatic, alkoxy, aryloxy, heteroaryloxy, (cycloaliphatic)oxy, (heterocycloaliphatic)oxy, aryl, heteroaryl, cycloaliphatic, or amino.

As used herein, an “aminoalkyl” refers to the formula (R^{X})₂N-alkyl- where R^{X} has been defined above.

As used herein, a “cyanoalkyl” refers to the formula (NC)-alkyl-.

As used herein, a “urea” group refers to the formula —NR^{X}—CO—NR^{Y}R^{Z} and a “thiourea” refers to the formula —NR^{X}—CS—NR^{Y}R^{Z} each when used terminally and —NR^{X}—CO—NR^{Y}— or —NR^{X}—CS—NR^{Y}—each when used internally, wherein R^{X}, R^{Y}, and R^{Z} have been defined above.

As used herein, a “guanidine” group refers to the formula —N═C(N(R^{X}R^{Y}))(N(R^{X}R^{Y})) or —NR^{X}—C(═NR^{X})NR^{X}R^{Y} wherein R^{X} and R^{Y} have been defined above.

As used herein, the term “amidino” group refers to the formula —C(NR^{X})NR^{X}R^{Y} wherein R^{X} and R^{Y} have been defined above.

As used herein, the term “vicinal” generally refers to the placement of substituents on a group that includes two or more carbon atoms, wherein the substituents are attached to adjacent carbon atoms.

As used herein, the term “geminal” generally refers to the placement of substituents on the same carbon atom.

As used herein, an “aliphatic chain” refers to a branched or straight aliphatic group (e.g., alkyl groups, alkenyl groups, or alkynyl groups). A straight aliphatic chain has the structure —[CH₂]ᵥ—, where v is one to twelve. A branched aliphatic chain is a straight aliphatic chain that is substituted with one or more aliphatic groups. A branched aliphatic chain has the structure -[CQQ]ᵥ-, where each Q is independently a hydrogen (H or —H) or an aliphatic group; however, Q shall be an aliphatic group in at least one instance. The term aliphatic chain includes alkyl chains, alkenyl chains, and alkynyl chains, where alkyl, alkenyl, and alkynyl are defined above.

The phrase “optionally substituted” is used interchangeably herein with the phrase “substituted or unsubstituted.” As described herein, compounds herein can optionally be substituted with one or more substituents, as illustrated generally above, or as exemplified by particular classes, subclasses, and species within this description. As described herein, the variables R, X, L, and Z, and other variables contained in Formula (I) or (II) described herein encompass specific groups, for example, alkyl and aryl. Unless otherwise noted, each of the specific groups within the variables R, X, L, and Z, and other variables contained therein can be optionally substituted with one or more substituents described herein. Each substituent of a specific group is further optionally substituted with one to three halo, cyano, oxo, alkoxy, hydroxy, amino, nitro, aryl, cycloaliphatic, heterocycloaliphatic, heteroaryl, haloalkyl, and/or alkyl. For instance, an alkyl group can be substituted with alkylsulfanyl and the alkylsulfanyl can be optionally substituted with one to three halo, cyano, oxo, alkoxy, hydroxy, amino, nitro, aryl, haloalkyl, and alkyl. As an additional example, the cycloalkyl portion of a (cycloalkyl)carbonylamino can be optionally substituted with one to three halo, cyano, alkoxy, hydroxy, nitro, haloalkyl, and alkyl. When two alkoxy groups are bound to the same atom or adjacent atoms, the two alkoxy groups can form a ring together with the atom(s) to which they are bound.

As used herein, the term “substituted,” whether preceded by the term “optionally” or not, refers generally to the replacement of one or more hydrogen atoms in a given chemical structure with the radical of a specified substituent. Specific substituents are defined above and described below within the compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position. A ring substituent, such as a heterocycloalkyl, can be bound to another ring, such as a cycloalkyl, to form a spiro-bicyclic ring system, for example, both rings share one common atom. Non-limiting examples of spiro heterocycloalkyls include

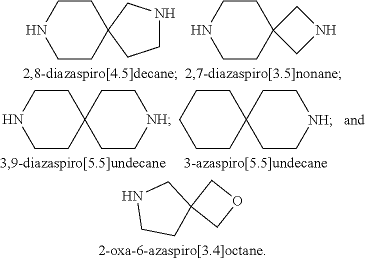

Spiro compounds depicted with overlapping rings indicate that the spirocyclic rings can bond at any vertex. For instance, in the spiro group

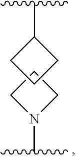

the two rings can bond at any of the three available vertex atoms in either ring.

As one of ordinary skill in the art will recognize, combinations of substituents envisioned by this description are those combinations that result in the formation of stable or chemically feasible compounds.

As used herein, the phrase “stable or chemically feasible” refers to compounds that are not substantially altered when subjected to conditions to allow for their production, detection, recovery, purification, and/or use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

As used herein, an “effective amount” is defined as the amount required to confer a therapeutic effect on the treated subject or patient, and is typically determined based on age, surface area, weight, and/or condition of the subject or patient. The interrelationship between dosages for animals and humans (based on milligrams per meter squared of body surface) is described by Freireich et al., Cancer Chemother. Rep., 50: 219 (1966). Body surface area may be approximately determined from height and weight of the subject or patient. See, e.g., Scientific Tables, Geigy Pharmaceuticals, Ardsley, New York, 537 (1970). As used herein, “patient” refers to an animal, alternatively a mammal, including a human.

The terms “pharmaceutical formulation” and “pharmaceutical composition” refer to preparations that are in such form as to permit the biological activity of the active ingredient to be effective, and that contain no additional components that are unacceptably toxic to an individual or subject to which the formulation or composition would be administered. Such formulations or compositions may be sterile.

The term “excipients” as used herein includes pharmaceutically acceptable excipients, carriers, vehicles, or stabilizers that are nontoxic to the subject, cell, or mammal being exposed thereto at the dosages and concentrations employed. In certain embodiments, the physiologically acceptable excipient is an aqueous pH buffered solution.

The terms “treating” or “treatment” of a disease refer to executing a protocol, which may include administering one or more therapeutic agent to a subject and/or an individual (human or otherwise), in an effort to obtain beneficial or desired results in the subject or individual, including clinical results. In certain embodiments, beneficial or desired clinical results include, but are not limited to, alleviation or amelioration of one or more symptoms, diminishment of extent of disease, stabilized (i.e., not worsening) state of disease, preventing spread of disease, delay or slowing of disease progression, amelioration or palliation of the disease state, and remission (whether partial or total). In certain embodiments, “treatment” can also mean prolonging survival as compared to expected survival of an individual not receiving treatment. In certain embodiments, “treating” and “treatment” may occur by administration of one dose of a therapeutic agent or therapeutic agents, or may occur upon administration of a series of doses of a therapeutic agent or therapeutic agents. In certain embodiments, “treating” or “treatment” does not require complete alleviation of signs or symptoms, and does not require a cure. In certain embodiments, “treatment” can also refer to clinical intervention, such as administering one or more therapeutic agents to a subject and/or an individual, designed to alter the natural course of the subject, individual, or cell being treated (i.e., to alter the course of the individual or cell that would occur in the absence of the clinical intervention). In certain embodiments, the term “therapeutic agent” can refer to a compound or drug that induces the proteolytic degradation of BRAF or compositions thereof.

The term “individual,” “patient,” or “subject” refers to a mammal. In certain embodiments, a “mammal” for purposes of treatment includes humans; non-human primates; domestic and farm animals; and zoo, sports, or pet animals, such as dogs, horses, rabbits, cattle, pigs, hamsters, gerbils, mice, ferrets, rats, cats, etc. In some embodiments, the individual or subject is human.

As used herein, the term “about” means within +10% of a stated value. For example, a dose that is about 100 mg/kg provides that the dose can be 90 mg/kg to 110 mg/kg. By way of further example, an amount of an additional therapeutic agent ranging from about 50% to about 100% provides that the amount of additional therapeutic agent ranges from 45-55% to 90-110%. A person of skill in the art will appreciate the scope and application of the term “about” when used to describe other stated values disclosed herein.

Unless otherwise stated, structures depicted herein also are meant to include all isomeric (e.g., enantiomeric, diastereomeric, and geometric (or conformational)) forms of the chemical structure; for example, the (R)- and (S)-configurations for each asymmetric center, (Z)—and (E)-double bond isomers, and syn-/cis- and anti-/trans-conformational isomers. Therefore, single stereochemical isomers as well as enantiomeric, diastereomeric, and geometric (or conformational) mixtures of the present compounds are within the scope of the description. Alternatively, as used herein, “enantiomeric excess (ee)” refers to a dimensionless mol ratio describing the purity of chiral substances that contain, for example, a single stereogenic center. For instance, an enantiomeric excess of zero would indicate a racemic (e.g., 50:50 mixture of enantiomers, or no excess of one enantiomer over the other). By way of further example, an enantiomeric excess of ninety-nine would indicate a nearly stereopure enantiomeric compound (i.e., large excess of one enantiomer over the other). The percentage enantiomeric excess, % ee=([(R)-compound]−[(S)-compound])/([(R)-compound]+[(S)-compound])×100, where the (R)-compound>(S)-compound; or % ee=([(S)-compound]−[(R)-compound])/([(S)-compound]+[(R)-compound])×100, where the (S)-compound>(R)-compound. Also as used herein, “diastereomeric excess (de)” refers to a dimensionless mol ratio describing the purity of chiral substances that contain more than one stereogenic center. For example, a diastereomeric excess of zero would indicate an equimolar mixture of diastereoisomers. By way of further example, diastereomeric excess of ninety-nine would indicate a nearly stereopure diastereomeric compound (i.e., large excess of one diastereomer over the other). Diastereomeric excess may be calculated via a similar method to ee. As would be appreciated by a person of skill, de is usually reported as percent de (% de). % de may be calculated in a similar manner to % ee.

In certain embodiments, the compounds or inhibitors described herein have an ee, de, % ee, or % de greater than zero. For example, in certain embodiments, the compounds or inhibitors described herein have an ee, de, % ee, or % de of ten. In certain embodiments, the compounds or inhibitors described herein have an ee, de, % ee, or % de of twenty-five. In certain embodiments, the compounds or inhibitors described herein have an ee, de, % ee, or % de of fifty. In certain embodiments, the compounds or inhibitors described herein have an ee, de, % ee, or % de of seventy-five. In certain embodiments, the compounds or inhibitors described herein have an ee, de, % ee, or % de of ninety-nine.

In certain embodiments, the compounds or inhibitors described herein have an ee, de, % ee, or % de range from ninety to one hundred. In certain embodiments, the compounds or inhibitors described herein have an ee, de, % ee, or % de range from ninety-five to one hundred. In certain embodiments, the compounds or inhibitors described herein have an ee, de, % ee, or % de range from ninety-seven to one hundred. In certain embodiments, the compounds or inhibitors described herein have an ee, de, % ee, or % de range from ninety-eight to one hundred. In certain embodiments, the compounds or inhibitors described herein have an ee, de, % ee, or % de range from ninety-nine to one hundred.

In one embodiment of a compound or inhibitor described herein, the ee, de, % ee, or % de is one. In one embodiment of a compound or inhibitor described herein, the ee, de, % ee, or % de is two. In one embodiment of a compound or inhibitor described herein, the ee, de, % ee, or % de is three. In one embodiment of a compound or inhibitor described herein, the ee, de, % ee, or % de is four. In one embodiment of a compound or inhibitor described herein, the ee, de, % ee, or % de is five. In one embodiment of a compound or inhibitor described herein, the ee, de, % ee, or % de is six. In one embodiment of a compound or inhibitor described herein, the ee, de, % ee, or % de is seven. In one embodiment of a compound or inhibitor described herein, the ee, de, % ee, or % de is eight. In one embodiment of a compound or inhibitor described herein, the ee, de, % ee, or % de is nine. In one embodiment of a compound or inhibitor described herein, the ee, de, % ee, or % de is ten. In one embodiment of a compound or inhibitor described herein, the ee, de, % ee, or % de is eleven. In one embodiment of a compound or inhibitor described herein, the ee, de, % ee, or % de is twelve. In one embodiment of a compound or inhibitor described herein, the ee, de, % ee, or % de is thirteen. In one embodiment of a compound or inhibitor described herein, the ee, de, % ee, or % de is fourteen. In one embodiment of a compound or inhibitor described herein, the ee, de, % ee, or % de is fifteen. In one embodiment of a compound or inhibitor described herein, the ee, de, % ee, or % de is sixteen. In one embodiment of a compound or inhibitor described herein, the ee, de, % ee, or % de is seventeen. In one embodiment of a compound or inhibitor described herein, the ee, de, % ee, or % de is eighteen. In one embodiment of a compound or inhibitor described herein, the ee, de, % ee, or % de is nineteen. In one embodiment of a compound or inhibitor described herein, the ee, de, % ee, or % de is twenty. In one embodiment of a compound or inhibitor described herein, the ee, de, % ee, or % de is twenty-one. In one embodiment of a compound or inhibitor described herein, the ee, de, % ee, or % de is twenty-two. In one embodiment of a compound or inhibitor described herein, the ee, de, % ee, or % de is twenty-three. In one embodiment of a compound or inhibitor described herein, the ee, de, % ee, or % de is twenty-four. In one embodiment of a compound or inhibitor described herein, the ee, de, % ee, or % de is twenty-five. In one embodiment of a compound or inhibitor described herein, the ee, de, % ee, or % de is twenty-six. In one embodiment of a compound or inhibitor described herein, the ee, de, % ee, or % de is twenty-seven. In one embodiment of a compound or inhibitor described herein, the ee, de, % ee, or % de is twenty-eight. In one embodiment of a compound or inhibitor described herein, the ee, de, % ee, or % de is twenty-nine. In one embodiment of a compound or inhibitor described herein, the ee, de, % ee, or % de is thirty. In one embodiment of a compound or inhibitor described herein, the ee, de, % ee, or % de is thirty-one. In one embodiment of a compound or inhibitor described herein, the ee, de, % ee, or % de is thirty-two. In one embodiment of a compound or inhibitor described herein, the ee, de, % ee, or % de is thirty-three. In one embodiment of a compound or inhibitor described herein, the ee, de, % ee, or % de is thirty-four. In one embodiment of a compound or inhibitor described herein, the ee, de, % ee, or % de is thirty-five. In one embodiment of a compound or inhibitor described herein, the ee, de, % ee, or % de is thirty-six. In one embodiment of a compound or inhibitor described herein, the ee, de, % ee, or % de is thirty-seven. In one embodiment of a compound or inhibitor described herein, the ee, de, % ee, or % de is thirty-eight. In one embodiment of a compound or inhibitor described herein, the ee, de, % ee, or % de is thirty-nine. In one embodiment of a compound or inhibitor described herein, the ee, de, % ee, or % de is forty. In one embodiment of a compound or inhibitor described herein, the ee, de, % ee, or % de is forty-one. In one embodiment of a compound or inhibitor described herein, the ee, de, % ee, or % de is forty-two. In one embodiment of a compound or inhibitor described herein, the ee, de, % ee, or % de is forty-three. In one embodiment of a compound or inhibitor described herein, the ee, de, % ee, or % de is forty-four. In one embodiment of a compound or inhibitor described herein, the ee, de, % ee, or % de is forty-five. In one embodiment of a compound or inhibitor described herein, the ee, de, % ee, or % de is forty-six. In one embodiment of a compound or inhibitor described herein, the ee, de, % ee, or % de is forty-seven. In one embodiment of a compound or inhibitor described herein, the ee, de, % ee, or % de is forty-eight. In one embodiment of a compound or inhibitor described herein, the ee, de, % ee, or % de is forty-nine. In one embodiment of a compound or inhibitor described herein, the ee, de, % ee, or % de is fifty. In one embodiment of a compound or inhibitor described herein, the ee, de, % ee, or % de is fifty-one. In one embodiment of a compound or inhibitor described herein, the ee, de, % ee, or % de is fifty-two. In one embodiment of a compound or inhibitor described herein, the ee, de, % ee, or % de is fifty-three. In one embodiment of a compound or inhibitor described herein, the ee, de, % ee, or % de is fifty-four. In one embodiment of a compound or inhibitor described herein, the ee, de, % ee, or % de is fifty-five. In one embodiment of a compound or inhibitor described herein, the ee, de, % ee, or % de is fifty-six. In one embodiment of a compound or inhibitor described herein, the ee, de, % ee, or % de is fifty-seven. In one embodiment of a compound or inhibitor described herein, the ee, de, % ee, or % de is fifty-eight. In one embodiment of a compound or inhibitor described herein, the ee, de, % ee, or % de is fifty-nine. In one embodiment of a compound or inhibitor described herein, the ee, de, % ee, or % de is sixty. In one embodiment of a compound or inhibitor described herein, the ee, de, % ee, or % de is sixty-one. In one embodiment of a compound or inhibitor described herein, the ee, de, % ee, or % de is sixty-two. In one embodiment of a compound or inhibitor described herein, the ee, de, % ee, or % de is sixty-three. In one embodiment of a compound or inhibitor described herein, the ee, de, % ee, or % de is sixty-four. In one embodiment of a compound or inhibitor described herein, the ee, de, % ee, or % de is sixty-five. In one embodiment of a compound or inhibitor described herein, the ee, de, % ee, or % de is sixty-six. In one embodiment of a compound or inhibitor described herein, the ee, de, % ee, or % de is sixty-seven. In one embodiment of a compound or inhibitor described herein, the ee, de, % ee, or % de is sixty-eight. In one embodiment of a compound or inhibitor described herein, the ee, de, % ee, or % de is sixty-nine. In one embodiment of a compound or inhibitor described herein, the ee, de, % ee, or % de is seventy. In one embodiment of a compound or inhibitor described herein, the ee, de, % ee, or % de is seventy-one. In one embodiment of a compound or inhibitor described herein, the ee, de, % ee, or % de is seventy-two. In one embodiment of a compound or inhibitor described herein, the ee, de, % ee, or % de is seventy-three. In one embodiment of a compound or inhibitor described herein, the ee, de, % ee, or % de is seventy-four. In one embodiment of a compound or inhibitor described herein, the ee, de, % ee, or % de is seventy-five. In one embodiment of a compound or inhibitor described herein, the ee, de, % ee, or % de is seventy-six. In one embodiment of a compound or inhibitor described herein, the ee, de, % ee, or % de is seventy-seven. In one embodiment of a compound or inhibitor described herein, the ee, de, % ee, or % de is seventy-eight. In one embodiment of a compound or inhibitor described herein, the ee, de, % ee, or % de is seventy-nine. In one embodiment of a compound or inhibitor described herein, the ee, de, % ee, or % de is eighty. In one embodiment of a compound or inhibitor described herein, the ee, de, % ee, or % de is eighty-one. In one embodiment of a compound or inhibitor described herein, the ee, de, % ee, or % de is eighty-two. In one embodiment of a compound or inhibitor described herein, the ee, de, % ee, or % de is eighty-three. In one embodiment of a compound or inhibitor described herein, the ee, de, % ee, or % de is eighty-four. In one embodiment of a compound or inhibitor described herein, the ee, de, % ee, or % de is eighty-five. In one embodiment of a compound or inhibitor described herein, the ee, de, % ee, or % de is eighty-six. In one embodiment of a compound or inhibitor described herein, the ee, de, % ee, or % de is eighty-seven. In one embodiment of a compound or inhibitor described herein, the ee, de, % ee, or % de is eighty-eight. In one embodiment of a compound or inhibitor described herein, the ee, de, % ee, or % de is eighty-nine. In one embodiment of a compound or inhibitor described herein, the ee, de, % ee, or % de is ninety. In one embodiment of a compound or inhibitor described herein, the ee, de, % ee, or % de is ninety-one. In one embodiment of a compound or inhibitor described herein, the ee, de, % ee, or % de is ninety-two. In one embodiment of a compound or inhibitor described herein, the ee, de, % ee, or % de is ninety-three. In one embodiment of a compound or inhibitor described herein, the ee, de, % ee, or % de is ninety-four. In one embodiment of a compound or inhibitor described herein, the ee, de, % ee, or % de is ninety-five. In one embodiment of a compound or inhibitor described herein, the ee, de, % ee, or % de is ninety-six. In one embodiment of a compound or inhibitor described herein, the ee, de, % ee, or % de is ninety-seven. In one embodiment of a compound or inhibitor described herein, the ee, de, % ee, or % de is ninety-eight. In one embodiment of a compound or inhibitor described herein, the ee, de, % ee, or % de is ninety-nine. In one embodiment of a compound or inhibitor described herein, the ee, de, % ee, or % de is one hundred. In certain embodiments, compounds or inhibitors described within Table 1 herein have an ee, de, % ee, or % de as described within this paragraph. In certain embodiments, compounds or inhibitors described in the Examples and/or Biological Examples have an ee, de, % ee, or % de as described within this paragraph. Unless otherwise stated, all tautomeric forms of the compounds of this description are within the scope of this description. Additionally, unless otherwise stated, structures depicted herein also are meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this description. Such compounds are useful, for example, as analytical tools or probes in biological assays, or as therapeutic agents.

As used herein, the term “&1” means that a compound including the “&1” notation at a particular chemical element or atom (e.g., carbon) within the compound was prepared as a mixture of two stereoisomers at the noted chemical element or atom (e.g., a diastereomeric mixture having a de or % de as described above).

Chemical structures and nomenclature are derived from ChemDraw, version 21.0, Cambridge, MA.

It is noted that the use of the descriptors “first,” “second,” “third,” or the like is used to differentiate separate elements (e.g., solvents, reaction steps, processes, reagents, or the like) and may or may not refer to the relative order or relative chronology of the elements described.

### Compounds

In one embodiment, provided is a compound of Formula (I)

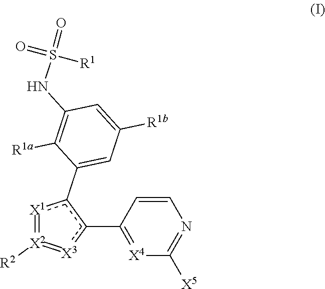

as described above. In certain embodiments, L is a linker according to -L¹-L²-L³-L⁴-L⁵-L⁶-L⁷- or -L⁷-L⁶-L⁵-L⁴-L³-L²-L¹-, wherein -L¹- is absent, —N(R¹⁰)—, —C(R¹¹)₂—, —C(O)—, —C₁₋₈ alkylene-, —C₂₋₈ alkynylene-, —C₆-C₁₀ aryl-, —C₄-C₁₀ heteroaryl-, Q¹, or Q²; each -L²-, -L³-, -L⁴-, and -L⁵- is independently, absent, —N(R¹⁰)—, —C(R¹¹)₂—, —C(O)—, —O—, —(CH₂—CH₂—O)₁₋₈—, —C₁₋₈ alkylene-, —C₂₋₈ alkynylene-, —C₆-C₁₀ aryl-, —C₄-C₁₀ heteroaryl-, Q¹, Q², or Q³; and each -L⁶- and -L⁷- is independently, absent, —N(R¹⁰)—, —C(R¹⁰)₂—, —C(O)—, —C(O)—N(R¹⁰)—, —N(R¹⁰)—C(O)—, or —C(R¹¹)₂—C(O)—N(R¹⁰)—.

In certain embodiments, R^{1b} is hydrogen. In certain embodiments, R^{1b} is halogen. In certain embodiments, X¹ is nitrogen; X² is carbon, X³ is sulfur; and X⁴ is carbon or nitrogen. In one embodiment, R¹ is alkyl; R^{1a} is fluoro; R² is —[Ar¹—Z¹]ₙ—Z²-Cy¹—Z³-L-UBM; n is zero; Z² is absent; and Cy¹ is an unsubstituted heterocycloalkylene. In one embodiment, Z³ is —C(O)—. In one embodiment, L¹ is Q¹. In one embodiment, L² is —C(O)—. In one embodiment, L³ is Q¹; and L⁴ is absent. In one embodiment, R¹ is —CH₂CH₂CH₃.

In certain embodiments, X¹ is carbon or nitrogen; X² is nitrogen, and X³ is carbon or nitrogen. In one embodiment, X¹ is carbon; X² is nitrogen, and X³ is nitrogen. In one embodiment, R¹ is alkyl; R^{1a} is fluoro; R² is —[Ar¹—Z¹]ₙ—Z²-Cy¹—Z³-L-UBM; Ar¹ is

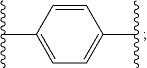 [SMILES: *c1ccc(*)cc1]

Z¹ is a bond; n is one; Z² and Z³ are absent; and Cy¹ is an unsubstituted heterocycloalkylene. In one embodiment, X⁴ is carbon and X⁵ is hydrogen. In one embodiment, L¹ is —C(R¹¹)₂—. In one embodiment, R¹¹ hydrogen. In one embodiment, L² is Q¹; and L³ and L⁴ are absent. In one embodiment, R¹ is —CH₂CH₂CH₃.

In certain embodiments, X¹ is nitrogen; X² is carbon, X³ is sulfur; and X⁴ is nitrogen. In one embodiment, R¹ is alkyl; R^{1a} is fluoro; R² is alkyl; n is zero; Z² is absent; and Cy¹ is an unsubstituted heterocycloalkylene. In one embodiment, Z³ is absent, —CH₂— or —C(O)—. In one embodiment, L¹ is Q¹, —C(R¹¹)₂—, or —CH₂CH₂CH₂CH₂CH₂—, or Q³. In one embodiment, L² is absent, Q¹, Q², —C(R¹¹)₂—, —C(O)—, or —O—. In one embodiment, L³ is absent, Q¹, or —CH₂CH₂CH₂—; and L⁴ is absent. In one embodiment, R¹ is —CH₂CH₂CH₃.

In certain embodiments, UBM binds an E3 ubiquitin ligase. In one embodiment, UBM binds SCFβ-TRCP, VHL, MDM2, IAP, or CRBN.

In certain embodiments, UBM binds VHL. In one embodiment, UBM binds VHL and has the following chemical formula

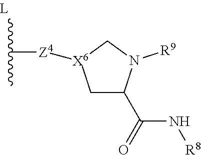

wherein

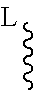

designates attachment to L; Z⁴ is a bond, —CH₂—, —C(O)—, —C(O)N(R¹²)—, or —N(R¹²)—, wherein R¹² is hydrogen or methyl; X⁶ is C—H or nitrogen; R¹ is alkyl, alkenyl, alkylene, alkynyl, aryl, cycloalkyl, heterocycloalkyl, heteroaryl, —S(O)(R¹³), or —S(O)₂(R¹³); wherein R¹³ is hydrogen, hydroxyl, alkyl, alkenyl, alkynyl, aryl, heterocycle, or heteroaryl, wherein each alkyl, alkenyl, alkylene, alkynyl, aryl, cycloalkyl, heterocycloalkyl, and heteroaryl is unsubstituted or substituted; R⁹ is hydrogen, unsubstituted or substituted C₁₋₈ alkyl, or -AA¹-AA²-R¹⁴ wherein each AA¹ and AA² is an amino acid residue, and R¹⁴is hydrogen or methyl. In one embodiment, UBM binds VHL and has the following chemical formula

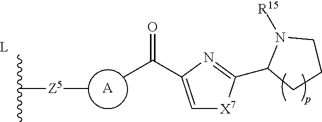

wherein

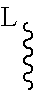

designates attachment to L; Z⁵ is a bond, —CH₂—, —C(O)—, —C(O)N(R¹²)—, —N(R¹²)—, or —O—, wherein R¹² is hydrogen or methyl; A is phenyl or C₄-heteroaryl; X⁷ is —CH₂, —NR¹², oxygen, or sulfur, wherein R¹² is hydrogen or methyl; p is zero or one; R¹⁵ is unsubstituted or substituted C₁₋₈ alkyl, -AA¹-AA²-R⁴, wherein each AA¹ and AA² is an amino acid residue, and R¹⁴ is hydrogen or methyl. In one embodiment, UBM binds VHL and has the following chemical formula

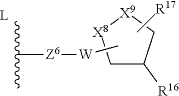

wherein

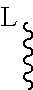

designates attachment to L; Z⁶ is a bond, —CH₂—, —C(O)—, —C(O)N(R¹²)—, or N(R¹²), wherein R¹² is hydrogen or methyl; W is

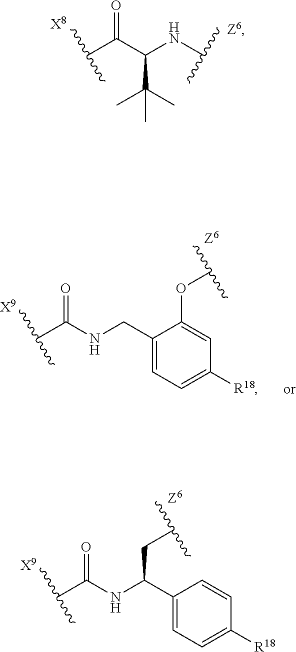

wherein,

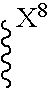

designates attachment to X⁸, wherein

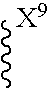

designates attachment to X⁹, and wherein,

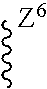

designates attachment to Z⁶; X⁸ is carbon or nitrogen; X⁹ is C—H or —CH₂—; R¹⁶ is hydrogen, —OH, halogen, —NH₂, —C₁₋₃ alkyl, —C₂₋₆ alkenyl, —C₂₋₆ alkynyl, —C₁₋₃ alkoxy, —C₁₋₃ thioalkyl, —C₁₋₃ alkylamine, —C₆₋₁₀ aryl, cycloalkyl, heterocycloalkyl, or heteroaryl; R¹⁷ is —C(O)N(H)—C₆₋₁₀ aralkyl, —C(O)CH(t-butyl)-N(H)—C₃₋₆ cycloalkyl, —C(O)—CH(t-butyl)-N(H)C(O)—C₃-C₆ cycloalkyl,

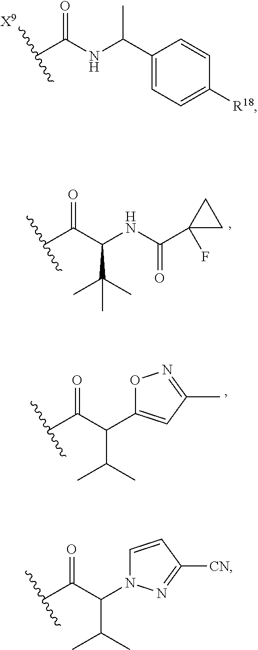

or -AA¹-AA²-R¹⁴, wherein each AA¹ and AA² is an amino acid residue and R¹⁴ is hydrogen or methyl; and R¹⁸ is halogen or

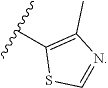 [SMILES: *c1scnc1C]

In certain embodiments, UBM binds CRBN. In one embodiment, UBM binds CRBN and has the following chemical formula

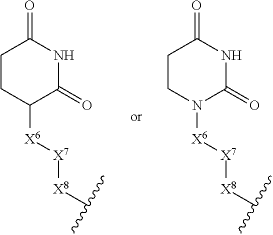

wherein
X⁶ absent or NR³; X⁷ absent or —C(O)—; and X⁸ is arylene or heteroarylene. In one embodiment, UBM binds CRBN and is selected from the group consisting of

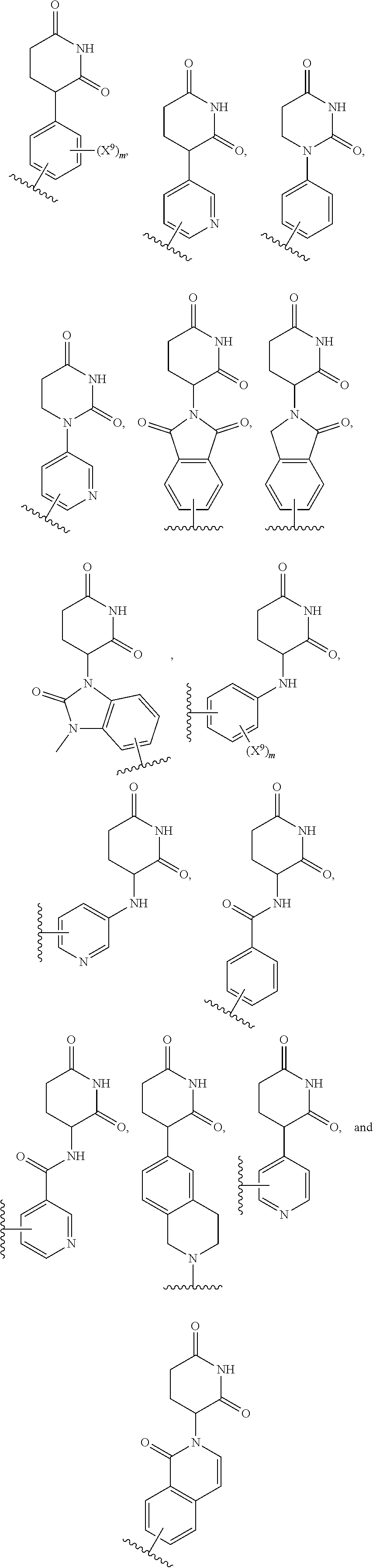

wherein
X⁹ is absent or halogen; and m is one, two, three, or four. In one embodiment, UBM binds CRBN and is

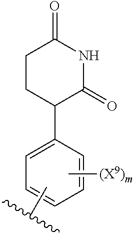

wherein X⁹ is absent or halogen; and m is one, two, three, or four.

In one embodiment, UBM binds CRBN and is

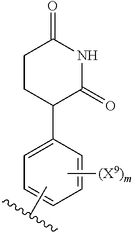

wherein X⁹ is absent. In one embodiment, UBM binds CRBN and is

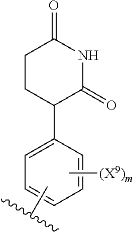

wherein X⁹ is halogen; and m is one. In one embodiment, UBM binds CRBN and is

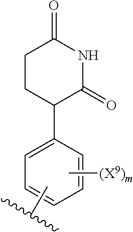

wherein X⁹ is halogen; and m is two.

In one embodiment, UBM binds CRBN and is

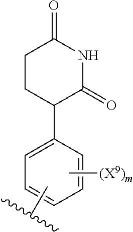

wherein X⁹ is halogen; and m is three. In one embodiment, UBM binds CRBN and is

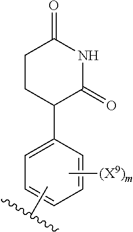

wherein X⁹ is halogen; and m is four. In one embodiment, UBM binds CRBN and is

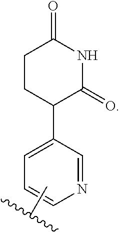

In one embodiment, UBM binds CRBN and is

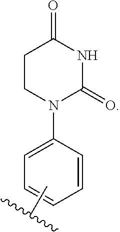

In one embodiment, UBM binds CRBN and is

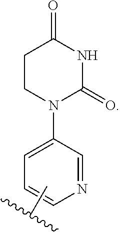

In one embodiment, UBM binds CRBN and is

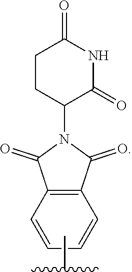

In one embodiment, UBM binds CRBN and is

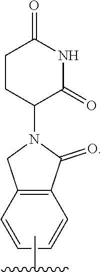

In one embodiment, UBM binds CRBN and is

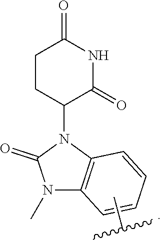

In one embodiment, UBM binds CRBN and is

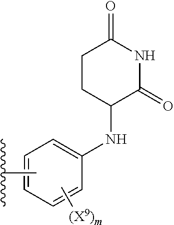

wherein X⁹ is absent or halogen; and m is one, two, three, or four. In one embodiment, UBM binds CRBN and is

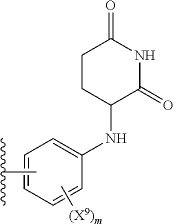

wherein X⁹ is absent. In one embodiment, UBM binds CRBN and is

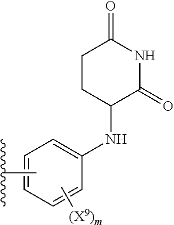

wherein X⁹ is halogen; and m is one. In one embodiment, UBM binds CRBN and is

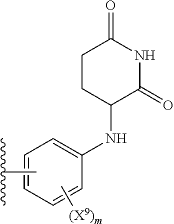

wherein X⁹ is halogen; and m is two. In one embodiment, UBM binds CRBN and is

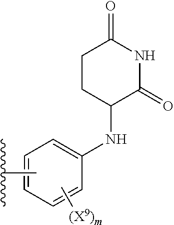

wherein X⁹ is halogen; and m is three. In one embodiment, UBM binds CRBN and is

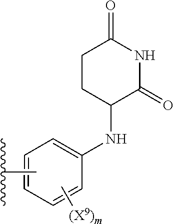

wherein X⁹ is halogen; and m is four. In one embodiment, UBM binds CRBN and is

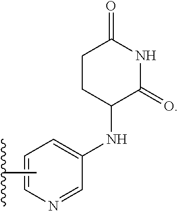

In one embodiment, UBM binds CRBN and is

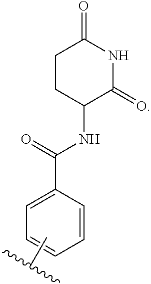

In one embodiment, UBM binds CRBN and is

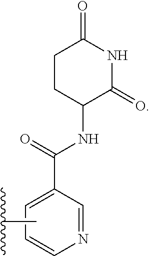

In one embodiment, UBM binds CRBN and is

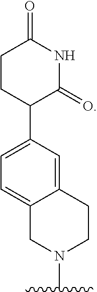 [SMILES: *N1CCc2cc(C3CCC(=O)NC3=O)ccc2C1]

In one embodiment, UBM binds CRBN and is

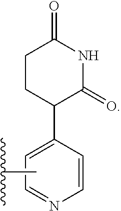

In one embodiment, UBM binds CRBN and is

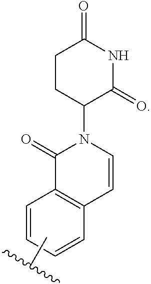

In one embodiment, provided is a compound of Formula (II)

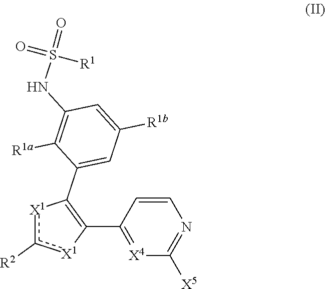

as described above. In certain embodiments, L is a linker according to -L¹-L²-L³-L⁴-L⁵-L⁶-L⁷- or -L⁷-L⁶-L⁵-L⁴-L³-L²-L¹-, wherein -L¹- is absent, —N(R¹⁰)—, —C(R¹¹)₂—, —C(O)—, —C₁₋₈ alkylene-, —C₂₋₈ alkynylene-, —C₆-C₁₀ aryl-, —C₄-C₁₀ heteroaryl-, Q¹, or Q²; each -L²-, -L³-, -L⁴-, and -L⁵- is independently, absent, —N(R¹⁰)—, —C(R¹¹)₂—, —C(O)—, —O—, —(CH₂—CH₂—O)₁₋₈—, —C₁₋₈ alkylene-, —C₂₋₈ alkynylene-, —C₆-C₁₀ aryl-, —C₄-C₁₀ heteroaryl-, Q¹, Q², or Q³; and each -L⁶- and -L⁷- is independently, absent, —N(R¹⁰)—, —C(R¹⁰)₂—, —C(O)—, —C(O)—N(R¹⁰)—, —N(R¹⁰)—C(O)—, or —C(R¹¹)₂—C(O)—N(R¹⁰)—.

In certain embodiments, R^{1b} is hydrogen. In certain embodiments, R^{1b} is halogen. In certain embodiments, X¹ is nitrogen; X³ is oxygen; and X⁴ is carbon or nitrogen. In certain embodiments, X¹ is oxygen; X³ is nitrogen; and X⁴ is carbon or nitrogen.

In certain embodiments, X¹ is nitrogen; X³ is sulfur; and X⁴ is carbon or nitrogen. In one embodiment, R¹ is alkyl; R^{1a} is fluoro; R² is —[Ar¹—Z¹]ₙ—Z²-Cy¹—Z³-L-UBM; n is zero; Z² is absent; and Cy¹ is an unsubstituted heterocycloalkylene. In one embodiment, Z³ is —C(O)—. In one embodiment, L¹ is Q¹. In one embodiment, L² is —C(O)—. In one embodiment, L³ is Q¹ and L⁴ is absent. In one embodiment, R¹ is —CH₂CH₂CH₃.

In certain embodiments, UBM binds an E3 ubiquitin ligase. In one embodiment, UBM binds SCFβ-TRCP, VHL, MDM2, IAP, or CRBN.

In certain embodiments, UBM binds VHL. In one embodiment, UBM binds VHL and has the following chemical formula

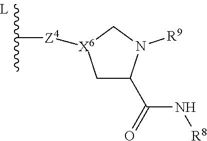

wherein

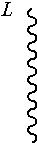

designates attachment to L; Z⁴ is a bond, —CH₂—, —C(O)—, —C(O)N(R¹²)—, or —N(R¹²)—, wherein R¹² is hydrogen or methyl; X⁶ is C—H or nitrogen; R⁸ is alkyl, alkenyl, alkylene, alkynyl, aryl, cycloalkyl, heterocycloalkyl, heteroaryl, —S(O)(R¹³), or —S(O)₂(R¹³); wherein R¹³ is hydrogen, hydroxyl, alkyl, alkenyl, alkynyl, aryl, heterocycle, or heteroaryl, wherein each alkyl, alkenyl, alkylene, alkynyl, aryl, cycloalkyl, heterocycloalkyl, and heteroaryl is unsubstituted or substituted; R⁹ is hydrogen, unsubstituted or substituted C₁₋₈ alkyl, or -AA¹-AA²-R¹⁴ wherein each AA¹ and AA² is an amino acid residue, and R¹⁴is hydrogen or methyl. In one embodiment, UBM binds VHL and has the following chemical formula

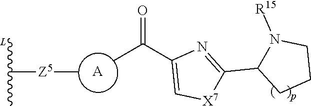

wherein

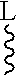

designates attachment to L; Z⁵ is a bond, —CH₂—, —C(O)—, —C(O)N(R¹²)—, —N(R¹²)—, or —O—, wherein R¹² is hydrogen or methyl; A is phenyl or C₄-heteroaryl; X⁷ is —CH₂, —NR¹², oxygen, or sulfur, wherein R¹² is hydrogen or methyl; p is zero or one; R¹⁵ is unsubstituted or substituted C₁₋₈ alkyl, -AA¹-AA²-R⁴, wherein each AA¹ and AA² is an amino acid residue, and R¹⁴ is hydrogen or methyl. In one embodiment, UBM binds VHL and has the following chemical formula

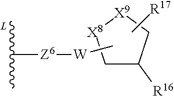

wherein

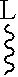

designates attachment to L; Z⁶ is a bond, —CH₂—, —C(O)—, —C(O)N(R¹²)—, or N(R¹²), wherein R¹² is hydrogen or methyl; W is

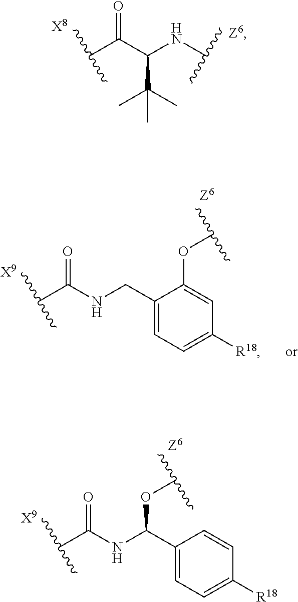

wherein,

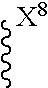

designates attachment to X⁸, wherein

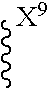

designates attachment to X⁹, and wherein,

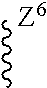

designates attachment to Z⁶; X⁸ is carbon or nitrogen; X⁹ is C—H or —CH₂—; R¹⁶ is hydrogen, —OH, halogen, —NH₂, —C₁₋₃ alkyl, —C₂₋₆ alkenyl, —C₂₋₆ alkynyl, —C₁₋₃ alkoxy, —C₁₋₃ thioalkyl, —C₁₋₃ alkylamine, —C₆₋₁₀ aryl, cycloalkyl, heterocycloalkyl, or heteroaryl; R¹⁷ is —C(O)N(H)—C₆₋₁₀ aralkyl, —C(O)CH(t-butyl)-N(H)—C₃₋₆ cycloalkyl, —C(O)—CH(t-butyl)-N(H)C(O)—C₃-C₆ cycloalkyl,

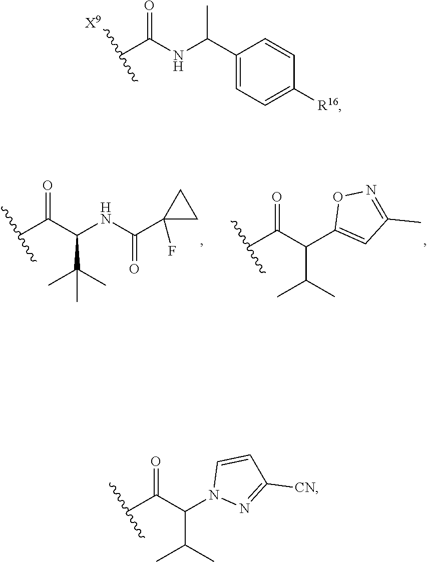

or -AA¹-AA²-R¹⁴, wherein each AA¹ and AA² is an amino acid residue and R¹⁴ is hydrogen or methyl; and R⁸ is halogen or

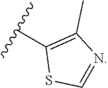 [SMILES: *c1scnc1C]

In certain embodiments, UBM binds CRBN. In one embodiment, UBM binds CRBN and has the following chemical formula

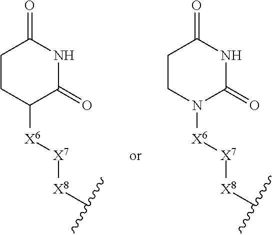

wherein
X⁶ is absent or NR³; X⁷ is absent or —C(O)—; and X⁸ is arylene or heteroarylene. In one embodiment, UBM binds CRBN and is selected from the group consisting of

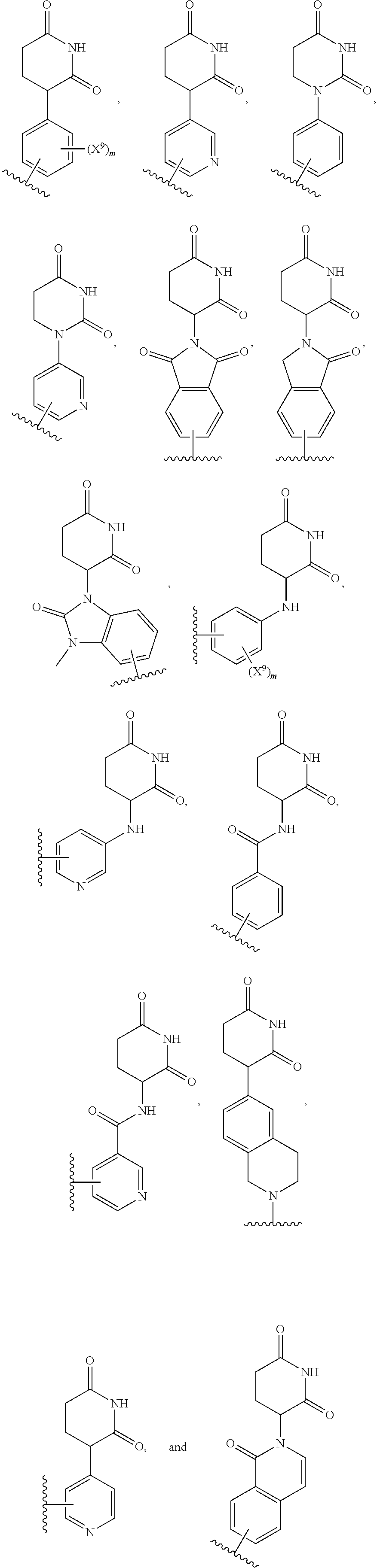

wherein X⁹ is absent or halogen; and m is one, two, three, or four. In one embodiment, UBM binds CRBN and is

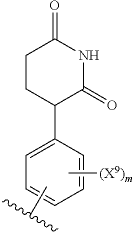

wherein X⁹ is absent or halogen; and m is one, two, three, or four. In one embodiment, UBM binds CRBN and is

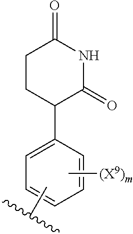

wherein X⁹ is absent. In one embodiment, UBM binds CRBN and is

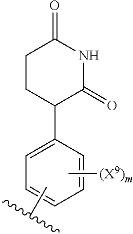

wherein X⁹ is halogen; and m is one. In one embodiment, UBM binds CRBN and is

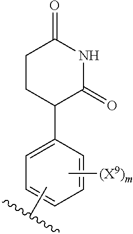

wherein X⁹ is halogen; and
m is two. In one embodiment, UBM binds CRBN and is

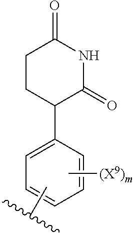

wherein X⁹ is halogen; and m is three. In one embodiment, UBM binds CRBN and is

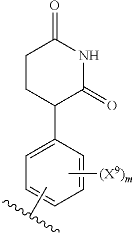

wherein
X⁹ is halogen; and m is four. In one embodiment, UBM binds CRBN and is

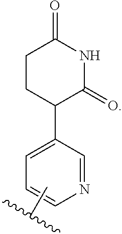

In one embodiment, UBM binds CRBN and is

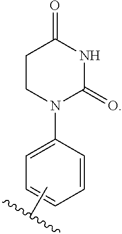

In one embodiment, UBM binds CRBN and is

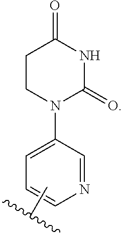

In one embodiment, UBM binds CRBN and is

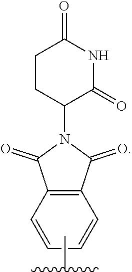

In one embodiment, UBM binds CRBN and is

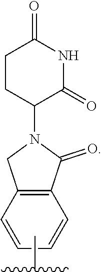

In one embodiment, UBM binds CRBN and is

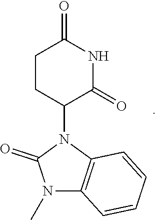 [SMILES: Cn1c(=O)n(C2CCC(=O)NC2=O)c2ccccc21]

In one embodiment, UBM binds CRBN and is

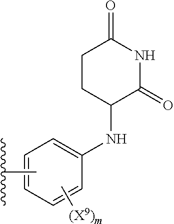

wherein
X⁹ is absent or halogen; and m is one, two, three, or four. In one embodiment, UBM binds CRBN and is

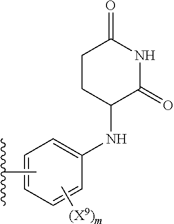

wherein X⁹ is absent. In one embodiment, UBM binds CRBN and is

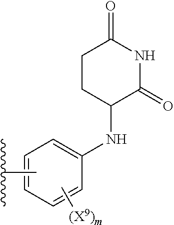

wherein X⁹ is halogen; and m is one. In one embodiment, UBM binds CRBN and is

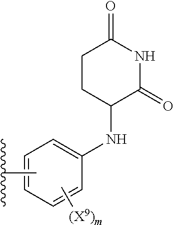

wherein X⁹ is halogen; and m is two. In one embodiment, UBM binds CRBN and is

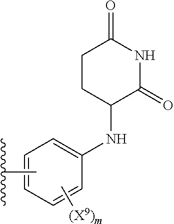

wherein X⁹ is halogen; and m is three. In one embodiment, UBM binds CRBN and is

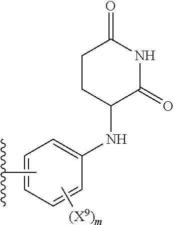

wherein X⁹ is halogen; and m is four. In one embodiment, UBM binds CRBN and is

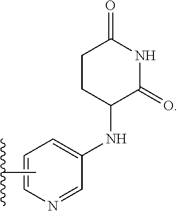

In one embodiment, UBM binds CRBN and is

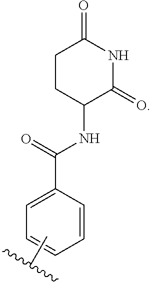

In one embodiment, UBM binds CRBN and is

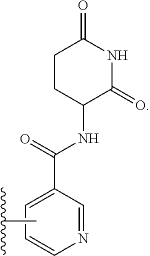

In one embodiment, UBM binds CRBN and is

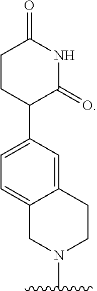 [SMILES: *N1CCc2cc(C3CCC(=O)NC3=O)ccc2C1]

In one embodiment, UBM binds CRBN and is

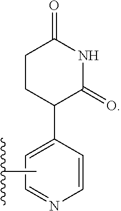

In one embodiment, UBM binds CRBN and is

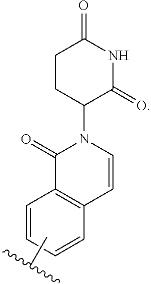

In certain embodiments of the compounds of Formula (I) or Formula (II), L includes at least one -Q¹-; at least one -Q²-; at least one -Q³-; at least one —C(R¹¹)₂—; or at least one C₁₋₈ alkylene-. In one embodiment, L is selected from the group consisting of
a. -Q¹-;
b. -Q¹-C(O)-Q¹-;
c. —C(R¹¹)₂-Q¹-;
d. -Q¹-C(R¹¹)₂-Q¹-;
e. —C(R¹¹)₂—O—;
f. -Q³-O—;
g. —C(R¹¹)₂-Q¹-Q-;
h. -Q¹-Q¹-;
i. —C(R¹¹)₂-Q²-;
j. —C₁₋₈ alkylene-;
k. —C(R¹¹)₂-Q¹-C₁₋₈ alkylene-;
l. -Q³-C(R¹¹)₂—;
m. —C(R¹¹)₂—;
n. -Q²-;
o. -Q¹-C(O)—C(R¹¹)₂—;
p. -Q¹-C(O)-Q³-O—;
q. -Q¹-C(O)—C(R¹¹)₂-Q¹-;
r. -Q¹-C(R¹¹)₂-Q³-O—; and
s. -Q¹-C(R¹¹)₂—.
   In one embodiment, L is -Q¹-. In one embodiment, L is -Q¹-C(O)-Q¹-. In one embodiment, L is —C(R¹¹)₂-Q¹-. In one embodiment, L is -Q¹-C(R¹¹)₂-Q¹-. In one embodiment, L is —C(R¹¹)₂—O—. In one embodiment, L is -Q³-O—. In one embodiment, L is —C(R¹¹)₂-Q¹-Q¹-. In one embodiment, L is -Q¹-Q¹-. In one embodiment, L is —C(R¹¹)₂-Q²-. In one embodiment, L is —C₁₋₈ alkylene-. In one embodiment, L is —C(R¹¹)₂-Q¹-C₁₋₈ alkylene-. In one embodiment, L is -Q³-C(R¹¹)₂—. In one embodiment, L is —C(R¹¹)₂—. In one embodiment, L is -Q²-. In one embodiment, L is -Q¹-C(O)—C(R¹¹)₂—. In one embodiment, L is -Q¹-C(O)-Q³-O—. In one embodiment, L is -Q¹-C(O)—C(R¹¹)₂-Q¹- In one embodiment, L is -Q¹-C(R¹¹)₂-Q³-O—. In one embodiment, L is -Q¹-C(R¹¹)₂—. In certain embodiments, -Q¹- is

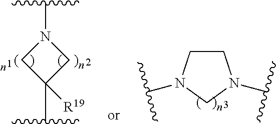

wherein R¹⁹ is hydrogen, hydroxyl, halogen, or unsubstituted alkyl, n¹ is one or two, n² is one or two, and n³ is one or two. In one embodiment, -Q¹- is selected from the group consisting of

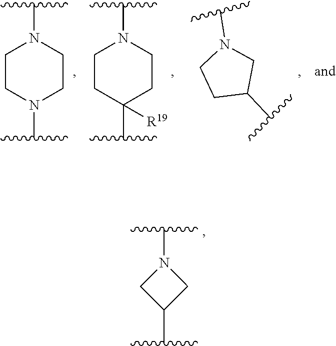

wherein R¹⁹ is hydrogen, hydroxyl, fluoro, or methyl. In one embodiment, -Q¹- is

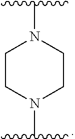 [SMILES: *N1CCN(*)CC1]

In one embodiment, -Q¹- is

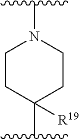

wherein R¹⁹ is hydrogen, hydroxyl, fluoro, or methyl. In one embodiment, -Q¹- is

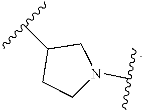 [SMILES: *C1CCN(*)C1]

In one embodiment, -Q¹- is

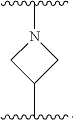 [SMILES: *C1CN(*)C1]

In certain embodiments, -Q²- is

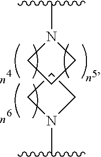

wherein n⁴ is one or two, n⁵ is one or two, and n⁶ is one or two. In one embodiment, -Q²- is selected from the group consisting of

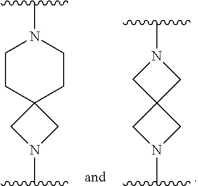 [SMILES: *N1CCC2(CC1)CN(*)C2 ; *N1CC2(C1)CN(*)C2]

In one embodiment, -Q²- is

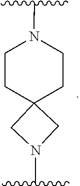 [SMILES: *N1CCC2(CC1)CN(*)C2]

In one embodiment, -Q²- is

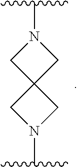 [SMILES: *N1CC2(C1)CN(*)C2]

In certain embodiments, -Q³- is

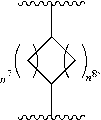

wherein n⁷ is one or two, and n⁸ is one or two. In one embodiment, -Q³- is selected from the group consisting of

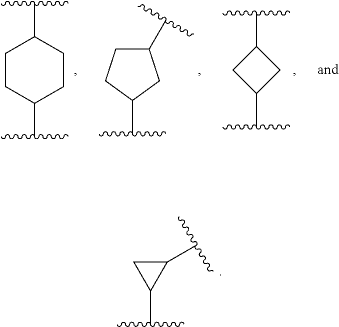

In one embodiment, -Q³- is

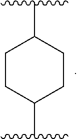 [SMILES: *C1CCC(*)CC1]

In one embodiment, -Q³- is

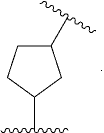 [SMILES: *C1CCC(*)C1]

In one embodiment, -Q³- is

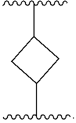 [SMILES: *C1CC(*)C1]

In one embodiment, -Q³- is

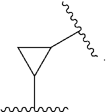 [SMILES: *C1CC1*]

In certain embodiments, each R¹¹ is, independently, hydrogen or methyl. In certain embodiments, both R¹¹ are hydrogen; one R¹¹ is hydrogen and one R¹¹ is methyl; or both R¹¹ are methyl. In certain embodiments, —C₁₋₈ alkylene- is selected from the group consisting of —CH₂—, —CH₂CH₂—, —CH₂CH₂CH₂—, —CH₂CH₂CH₂CH₂—, —CH₂CH₂CH₂CH₂CH₂—, —CH₂CH₂CH₂CH₂CH₂CH₂—, —CH₂CH₂CH₂CH₂CH₂CH₂CH₂—, and —CH₂CH₂CH₂CH₂CH₂CH₂CH₂CH₂—. In one embodiment, —C₁₋₈ alkylene- is —CH₂—. In one embodiment, —C₁₋₈ alkylene- is —CH₂CH₂—. In one embodiment, —C₁₋₈ alkylene- is —CH₂CH₂CH₂—. In one embodiment, —C₁₋₈ alkylene- is —CH₂CH₂CH₂CH₂—. In one embodiment, —C₁₋₈ alkylene- is —CH₂CH₂CH₂CH₂CH₂—. In one embodiment, —C₁₋₈ alkylene- is —CH₂CH₂CH₂CH₂CH₂CH₂—. In one embodiment, —C₁₋₈ alkylene- is —CH₂CH₂CH₂CH₂CH₂CH₂CH₂—. In one embodiment, —C₁₋₈ alkylene- is —CH₂CH₂CH₂CH₂CH₂CH₂CH₂CH₂—. In certain embodiments, the linker L is selected from

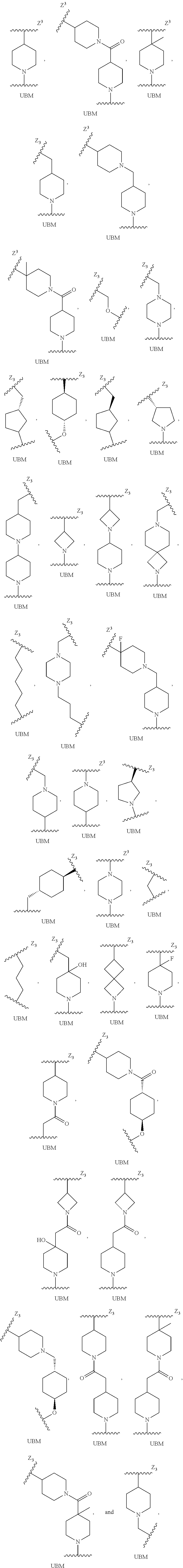

wherein

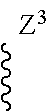

indicates attachment to Z³, and

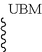

indicates attachment to UBM. In one embodiment, the linker L is

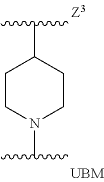 [SMILES: *C1CCN(*)CC1]

wherein

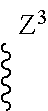

indicates attachment to Z³, and

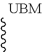

indicates attachment to UBM. In one embodiment, the linker L is

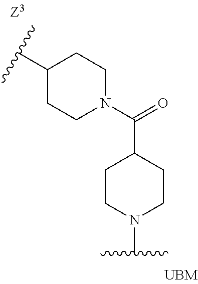 [SMILES: *C1CCN(C(=O)C2CCN(*)CC2)CC1]

wherein

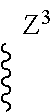

indicates attachment to Z³, and

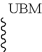

indicates attachment to UBM. In one embodiment, the linker L is

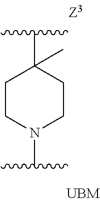 [SMILES: *N1CCC(*)(C)CC1]

wherein

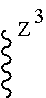

indicates attachment to Z³, and

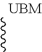

indicates attachment to UBM. In one embodiment, the linker L is

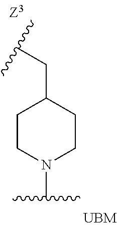 [SMILES: *CC1CCN(*)CC1]

wherein

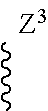

indicates attachment to Z³, and

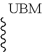

indicates attachment to UBM. In one embodiment, the linker L is

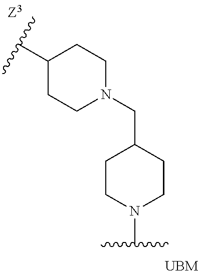 [SMILES: *C1CCN(CC2CCN(*)CC2)CC1]

wherein

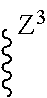

indicates attachment to Z³, and

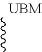

indicates attachment to UBM. In one embodiment, the linker L is

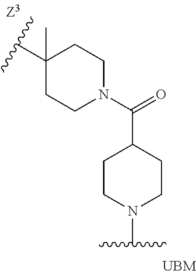 [SMILES: *N1CCC(C(=O)N2CCC(*)(C)CC2)CC1]

wherein

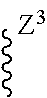

indicates attachment to Z³, and

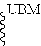

indicates attachment to UBM. In one embodiment, the linker L is

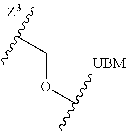 [SMILES: *CO*]

wherein

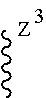

indicates attachment to Z³, and

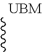

indicates attachment to UBM.

In one embodiment, the linker L is

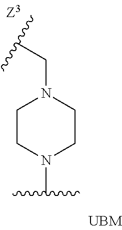 [SMILES: *CN1CCN(*)CC1]

wherein

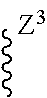

indicates attachment to Z³, and

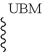

indicates attachment to UBM. In one embodiment, the linker L is

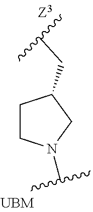 [SMILES: *C[C@H]1CCN(*)C1]

wherein

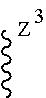

indicates attachment to Z³, and

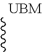

indicates attachment to UBM. In one embodiment, the linker L is

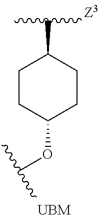 [SMILES: *O[C@H]1CC[C@H](*)CC1]

wherein

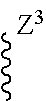

indicates attachment to Z³, and

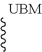

indicates attachment to UBM. In one embodiment, the linker L is

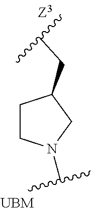 [SMILES: *C[C@@H]1CCN(*)C1]

wherein

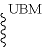

indicates attachment to Z³, and

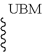

indicates attachment to UBM. In one embodiment, the linker L is

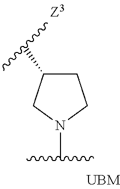 [SMILES: *[C@@H]1CCN(*)C1]

wherein

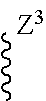

indicates attachment to Z³, and

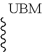

indicates attachment to UBM. In one embodiment, the linker L is

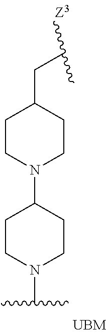 [SMILES: *CC1CCN(C2CCN(*)CC2)CC1]

wherein

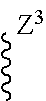

indicates attachment to Z³, and

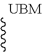

indicates attachment to UBM. In one embodiment, the linker L is

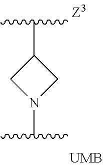 [SMILES: *C1CN(*)C1]

wherein

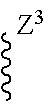

indicates attachment to Z³, and

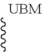

indicates attachment to UBM. In one embodiment, the linker L is

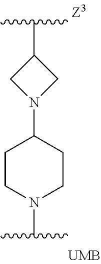 [SMILES: *C1CN(C2CCN(*)CC2)C1]

wherein

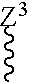

indicates attachment to Z³, and

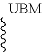

indicates attachment to UBM. In one embodiment, the linker L is

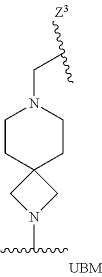 [SMILES: *CN1CCC2(CC1)CN(*)C2]

wherein

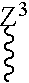

indicates attachment to Z³, and

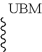

indicates attachment to UBM. In one embodiment, the linker L is

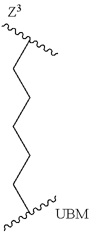 [SMILES: *CCCCC*]

wherein

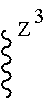

indicates attachment to Z³, and

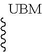

indicates attachment to UBM. In one embodiment, the linker L is

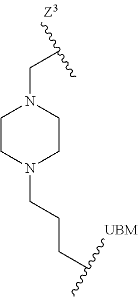 [SMILES: *CCCN1CCN(C*)CC1]

wherein

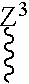

indicates attachment to Z³, and

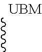

indicates attachment to UBM. In one embodiment, the linker L is

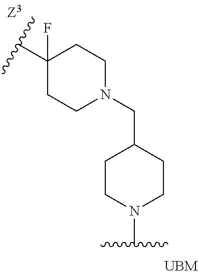 [SMILES: *N1CCC(CN2CCC(*)(F)CC2)CC1]

wherein

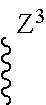

indicates attachment to Z³, and

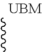

indicates attachment to UBM. In one embodiment, the linker L is

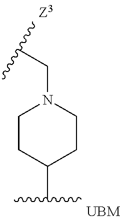 [SMILES: *CN1CCC(*)CC1]

wherein

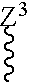

indicates attachment to Z³, and

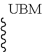

indicates attachment to UBM. In one embodiment, the linker L is

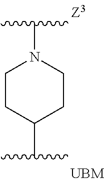 [SMILES: *C1CCN(*)CC1]

wherein

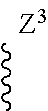

indicates attachment to Z³, and

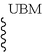

indicates attachment to UBM. In one embodiment, the linker L is

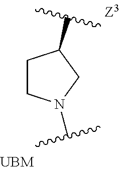 [SMILES: *[C@@H]1CCN(*)C1]

wherein

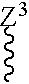

indicates attachment to Z³, and

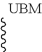

indicates attachment to UBM. In one embodiment, the linker L is

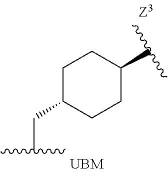 [SMILES: *C[C@H]1CC[C@H](*)CC1]

wherein

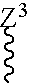

indicates attachment to Z³, and

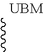

indicates attachment to UBM. In one embodiment, the linker L is

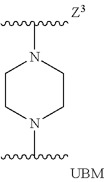 [SMILES: *N1CCN(*)CC1]

wherein

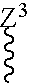

indicates attachment to Z³, and

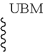

indicates attachment to UBM. In one embodiment, the linker L is

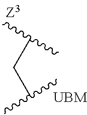 [SMILES: *C*]

wherein

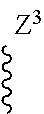

indicates attachment to Z³, and

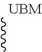

indicates attachment to UBM. In one embodiment, the linker L is

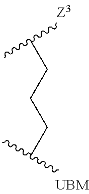 [SMILES: *CCC*]

wherein

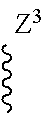

indicates attachment to Z³, and

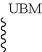

indicates attachment to UBM. In one embodiment, the linker L is

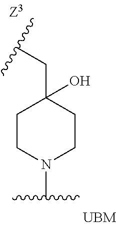 [SMILES: *CC1(O)CCN(*)CC1]

wherein

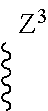

indicates attachment to Z³, and

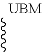

indicates attachment to UBM. In one embodiment, the linker L is

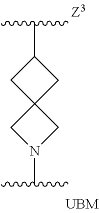 [SMILES: *C1CC2(C1)CN(*)C2]

wherein

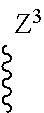

indicates attachment to Z³, and

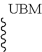

indicates attachment to UBM. In one embodiment, the linker L is

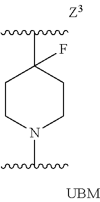 [SMILES: *N1CCC(*)(F)CC1]

wherein

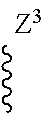

indicates attachment to Z³, and

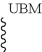

indicates attachment to UBM. In one embodiment, the linker L is

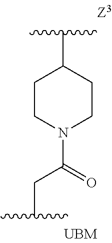 [SMILES: *CC(=O)N1CCC(*)CC1]

wherein

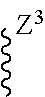

indicates attachment to Z³, and

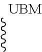

indicates attachment to UBM. In one embodiment, the linker L is

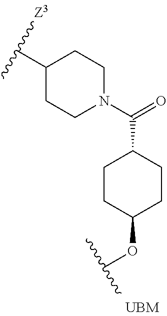 [SMILES: *O[C@H]1CC[C@H](C(=O)N2CCC(*)CC2)CC1]

wherein

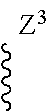

indicates attachment to Z³, and

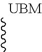

indicates attachment to UBM. In one embodiment, the linker L is

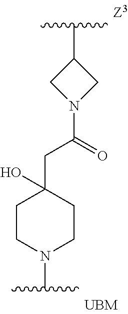 [SMILES: *C1CN(C(=O)CC2(O)CCN(*)CC2)C1]

wherein

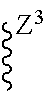

indicates attachment to Z³, and

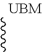

indicates attachment to UBM. In one embodiment, the linker L is

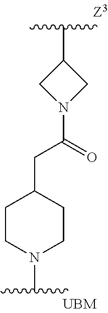 [SMILES: *C1CN(C(=O)CC2CCN(*)CC2)C1]

wherein

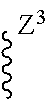

indicates attachment to Z³, and

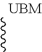

indicates attachment to UBM. In one embodiment, the linker L is

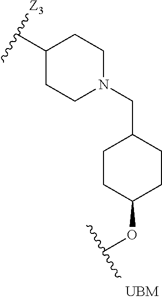

wherein

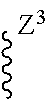

indicates attachment to Z³, and

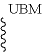

indicates attachment to UBM. In one embodiment, the linker L is

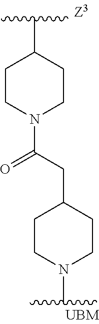 [SMILES: *C1CCN(C(=O)CC2CCN(*)CC2)CC1]

wherein

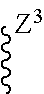

indicates attachment to Z³, and

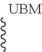

indicates attachment to UBM.

In one embodiment, the linker L is

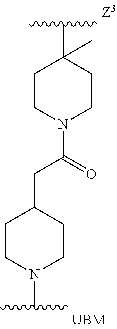 [SMILES: *N1CCC(CC(=O)N2CCC(*)(C)CC2)CC1]

wherein

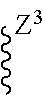

indicates attachment to Z³, and

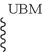

indicates attachment to UBM. In one embodiment, the linker L is

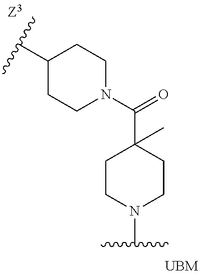 [SMILES: *C1CCN(C(=O)C2(C)CCN(*)CC2)CC1]

wherein

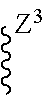

indicates attachment to Z³, and

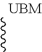

indicates attachment to UBM. In one embodiment, the linker L is

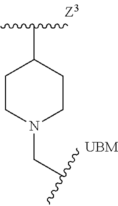 [SMILES: *CN1CCC(*)CC1]

wherein

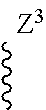

indicates attachment to Z³, and

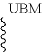

indicates attachment to UBM.

In certain embodiments, the compound of Formula (I) is selected from the group consisting of (S)—N-(3-(5-(2-aminopyrimidin-4-yl)-2-(1-(1-(1-(5-(2,6-dioxopiperidin-3-yl)pyridin-2-yl)piperidine-4-carbonyl)piperidine-4-carbonyl)piperidin-4-yl)thiazol-4-yl)-2-fluorophenyl)propane-1-sulfonamide, (S)—N-(3-(5-(2-aminopyrimidin-4-yl)-2-(1-(1-(1-(5-(2,6-dioxopiperidin-3-yl)pyridin-2-yl)-4-methylpiperidine-4-carbonyl)piperidine-4-carbonyl)piperidin-4-yl)thiazol-4-yl)-2-fluorophenyl)propane-1-sulfonamide, (S)—N-(3-(5-(2-aminopyrimidin-4-yl)-2-(1-(1-(1-(5-(2,6-dioxopiperidin-3-yl)pyridin-2-yl)piperidine-4-carbonyl)-4-methylpiperidine-4-carbonyl)piperidin-4-yl)thiazol-4-yl)-2-fluorophenyl)propane-1-sulfonamide, (R)—N-(3-(5-(2-aminopyrimidin-4-yl)-2-(1-(1-(1-(4-(2,6-dioxopiperidin-3-yl)phenyl)piperidine-4-carbonyl)-4-methylpiperidine-4-carbonyl)piperidin-4-yl)thiazol-4-yl)-2-fluorophenyl)propane-1-sulfonamide, (S)—N-(3-(2-(1-(1-(1-(4-(2,6-dioxopiperidin-3-yl)phenyl)piperidine-4-carbonyl)piperidine-4-carbonyl)piperidin-4-yl)-5-(pyrimidin-4-yl)thiazol-4-yl)-2-fluorophenyl)propane-1-sulfonamide, (S)—N-(3-(2-(1-(1-(1-(5-(2,6-dioxopiperidin-3-yl)pyridin-2-yl)piperidine-4-carbonyl)-4-methylpiperidine-4-carbonyl)piperidin-4-yl)-5-(pyrimidin-4-yl)thiazol-4-yl)-2-fluorophenyl)propane-1-sulfonamide, and (S)—N-(3-(2-(1-(1-(1-(5-(2,6-dioxopiperidin-3-yl)pyridin-2-yl)piperidine-4-carbonyl)-4-methylpiperidine-4-carbonyl)piperidin-4-yl)-5-(pyridin-4-yl)thiazol-4-yl)-2-fluorophenyl)propane-1-sulfonamide. In certain embodiments, the compound of Formula (I) is selected from the group consisting of (R)—N-(3-(5-(2-aminopyrimidin-4-yl)-2-(3-(2-(4-(3-(2-(2,6-dioxopiperidin-3-yl)-1,3-dioxoisoindolin-4-yl)propyl)piperazin-1-yl)acetyl)-3-azaspiro[5.5]undecan-9-yl)thiazol-4-yl)-2-fluorophenyl)propane-1-sulfonamide, N-(3-(5-(2-aminopyrimidin-4-yl)-2-(1-((1S,4r)-4-((6-((S)-2,6-dioxopiperidin-3-yl)-3,4-dihydroisoquinolin-2(1H)-yl)methyl)cyclohexane-1-carbonyl)piperidin-4-yl)thiazol-4-yl)-2-fluorophenyl)propane-1-sulfonamide, (S)—N-(3-(2-(1-(1-((1-(5-(2,6-dioxopiperidin-3-yl)pyridin-2-yl)piperidin-4-yl)methyl)piperidine-4-carbonyl)piperidin-4-yl)-5-(pyrimidin-4-yl)thiazol-4-yl)-2-fluorophenyl)propane-1-sulfonamide, (R)—N-(3-(5-(2-aminopyrimidin-4-yl)-2-(1-(2-(4-(2-(2,6-dioxopiperidin-3-yl)-1,3-dioxoisoindolin-5-yl)piperazin-1-yl)acetyl)-4-methylpiperidin-4-yl)thiazol-4-yl)-2-fluorophenyl)propane-1-sulfonamide, (R)—N-(3-(5-(2-aminopyrimidin-4-yl)-2-(1-(2-(1′-(2-(2,6-dioxopiperidin-3-yl)-1,3-dioxoisoindolin-5-yl)-[1,4′-bipiperidin]-4-yl)acetyl)-4-methylpiperidin-4-yl)thiazol-4-yl)-2-fluorophenyl)propane-1-sulfonamide, (S)—N-(3-(5-(2-aminopyrimidin-4-yl)-2-(1-(1-((1-(5-(2,6-dioxopiperidin-3-yl)pyridin-2-yl)piperidin-4-yl)methyl)piperidine-4-carbonyl)-4-methylpiperidin-4-yl)thiazol-4-yl)-2-fluorophenyl)propane-1-sulfonamide, (S)—N-(3-(5-(2-aminopyrimidin-4-yl)-2-(1-((1-(2-(6-(2,6-dioxopiperidin-3-yl)-3,4-dihydroisoquinolin-2(1H)-yl)acetyl)piperidin-4-yl)methyl)piperidin-4-yl)thiazol-4-yl)-2-fluorophenyl)propane-1-sulfonamide, (S)—N-(3-(5-(2-aminopyrimidin-4-yl)-2-(3-((1-(4-(2,6-dioxopiperidin-3-yl)phenyl)piperidin-4-yl)methyl)-3-azaspiro[5.5]undecan-9-yl)thiazol-4-yl)-2-fluorophenyl)propane-1-sulfonamide, (S)—N-(3-(2-(1-(1-((1-(5-(2,6-dioxopiperidin-3-yl)pyridin-2-yl)piperidin-4-yl)methyl)piperidine-4-carbonyl)piperidin-4-yl)-5-(pyridin-4-yl)thiazol-4-yl)-2-fluorophenyl)propane-1-sulfonamide, (R)—N-(3-(5-(2-aminopyrimidin-4-yl)-2-(1-(2-(1′-(2-(2,6-dioxopiperidin-3-yl)-1,3-dioxoisoindolin-5-yl)-[1,4′-bipiperidin]-4-yl)acetyl)piperidin-4-yl)thiazol-4-yl)-2-fluorophenyl)propane-1-sulfonamide, (R)—N-(3-(5-(2-aminopyrimidin-4-yl)-2-(1-(2-(4-(2-(2,6-dioxopiperidin-3-yl)-1,3-dioxoisoindolin-5-yl)piperazin-1-yl)acetyl)piperidin-4-yl)thiazol-4-yl)-2-fluorophenyl)propane-1-sulfonamide, (R)—N-(3-(5-(2-aminopyrimidin-4-yl)-2-(2-(1-(1-(1-(2-(2,6-dioxopiperidin-3-yl)-1,3-dioxoisoindolin-5-yl)piperidin-4-yl)azetidine-3-carbonyl)piperidin-4-yl)propan-2-yl)thiazol-4-yl)-2-fluorophenyl)propane-1-sulfonamide, N-(3-(5-(2-aminopyrimidin-4-yl)-2-(1-((1-((1R,4r)-4-(3-((R)-2,6-dioxopiperidin-3-yl)phenoxy)cyclohexane-1-carbonyl)piperidin-4-yl)methyl)piperidin-4-yl)thiazol-4-yl)-2-fluorophenyl)propane-1-sulfonamide, (R)—N-(3-(5-(2-aminopyrimidin-4-yl)-2-(3-((1-(5-(2,6-dioxopiperidin-3-yl)pyridin-2-yl)piperidin-4-yl)methyl)-3-azaspiro[5.5]undecan-9-yl)thiazol-4-yl)-2-fluorophenyl)propane-1-sulfonamide, (S)—N-(3-(5-(2-aminopyrimidin-4-yl)-2-(1-(1-(2-(1-(4-((2,6-dioxopiperidin-3-yl)amino)-2-fluorophenyl)-4-hydroxypiperidin-4-yl)acetyl)azetidin-3-yl)piperidin-4-yl)thiazol-4-yl)-2-fluorophenyl)propane-1-sulfonamide, (S)—N-(3-(5-(2-aminopyrimidin-4-yl)-2-(4-(1-(2-(1-(5-(2,6-dioxopiperidin-3-yl)pyridin-2-yl)piperidin-4-yl)acetyl)piperidin-4-yl)phenyl)thiazol-4-yl)-2-fluorophenyl)propane-1-sulfonamide, (S)—N-(3-(5-(2-aminopyrimidin-4-yl)-2-(4-(4-((1-(4-(2,6-dioxopiperidin-3-yl)phenyl)piperidin-4-yl)methyl)piperazin-1-yl)phenyl)thiazol-4-yl)-2-fluorophenyl)propane-1-sulfonamide, (S)—N-(3-(5-(2-aminopyrimidin-4-yl)-2-(4-(1-(2-(1-(4-((2,6-dioxopiperidin-3-yl)amino)phenyl)piperidin-4-yl)acetyl)piperidin-4-yl)phenyl)thiazol-4-yl)-2-fluorophenyl)propane-1-sulfonamide, (S)—N-(3-(5-(2-aminopyrimidin-4-yl)-2-(4-(1-(1-(5-(2,6-dioxopiperidin-3-yl)pyridin-2-yl)piperidine-4-carbonyl)piperidin-4-yl)phenyl)thiazol-4-yl)-2-fluorophenyl)propane-1-sulfonamide, (S)—N-(3-(5-(2-aminopyrimidin-4-yl)-2-(4-(1-((1-(4-(2,6-dioxopiperidin-3-yl)phenyl)piperidin-4-yl)methyl)piperidin-4-yl)phenyl)thiazol-4-yl)-2-fluorophenyl)propane-1-sulfonamide, N-(3-(5-(2-aminopyrimidin-4-yl)-2-(3-(((1S,4r)-4-((5-((S)-2,6-dioxopiperidin-3-yl)pyridin-2-yl)oxy)cyclohexyl)methyl)-3-azaspiro[5.5]undecan-9-yl)thiazol-4-yl)-2-fluorophenyl)propane-1-sulfonamide, (R)—N-(3-(5-(2-aminopyrimidin-4-yl)-2-(4-(4-(1-(4-(2,6-dioxopiperidin-3-yl)phenyl)piperidine-4-carbonyl)piperazin-1-yl)phenyl)thiazol-4-yl)-2-fluorophenyl)propane-1-sulfonamide, (R)-5-(4-(9-(5-(2-aminopyrimidin-4-yl)-4-(2-fluoro-3-(propylsulfonamido)phenyl)thiazol-2-yl)-3-azaspiro[5.5]undecane-3-carbonyl)piperidin-1-yl)-N-(2,6-dioxopiperidin-3-yl)picolinamide, N-(3-(5-(2-aminopyrimidin-4-yl)-2-(3-(2-((S)-1-(5-((S)-2,6-dioxopiperidin-3-yl)pyridin-2-yl)pyrrolidin-3-yl)ethyl)-3-azaspiro[5.5]undecan-9-yl)thiazol-4-yl)-2-fluorophenyl)propane-1-sulfonamide, (S)—N-(3-(5-(2-aminopyrimidin-4-yl)-2-(1-(1-(2-(1-(4-(2,6-dioxopiperidin-3-yl)phenyl)piperidin-4-yl)acetyl)azetidin-3-yl)piperidin-4-yl)thiazol-4-yl)-2-fluorophenyl)propane-1-sulfonamide, (S)—N-(3-(5-(2-aminopyrimidin-4-yl)-2-(3-(1-(5-(2,6-dioxopiperidin-3-yl)pyridin-2-yl)piperidine-4-carbonyl)-3-azaspiro[5.5]undecan-9-yl)thiazol-4-yl)-2-fluorophenyl)propane-1-sulfonamide, (S)—N-(3-(5-(2-aminopyrimidin-4-yl)-2-(4-(1-((1-(5-(2,6-dioxopiperidin-3-yl)pyridin-2-yl)piperidin-4-yl)methyl)piperidin-4-yl)phenyl)thiazol-4-yl)-2-fluorophenyl)propane-1-sulfonamide, (S)—N-(3-(2-(1-(1-(1-(4-(2,6-dioxopiperidin-3-yl)phenyl)piperidine-4-carbonyl)piperidine-4-carbonyl)piperidin-4-yl)-5-(pyridin-4-yl)thiazol-4-yl)-2-fluorophenyl)propane-1-sulfonamide, (S)—N-(3-(2-(1-(1-((1-(5-(2,6-dioxopiperidin-3-yl)pyridin-2-yl)piperidin-4-yl)methyl)piperidine-4-carbonyl)piperidin-4-yl)-5-(2-(methylamino)pyrimidin-4-yl)thiazol-4-yl)-2-fluorophenyl)propane-1-sulfonamide, (R)—N-(3-(5-(2-aminopyrimidin-4-yl)-2-(2-(2-(1′-(2-(2,6-dioxopiperidin-3-yl)-1,3-dioxoisoindolin-5-yl)-[1,4′-bipiperidin]-4-yl)acetyl)-2-azaspiro[3.5]nonan-7-yl)thiazol-4-yl)-2-fluorophenyl)propane-1-sulfonamide, (S)—N-(3-(2-(1-(1-(1-(5-(2,6-dioxopiperidin-3-yl)pyridin-2-yl)piperidine-4-carbonyl)piperidine-4-carbonyl)piperidin-4-yl)-5-(pyridin-4-yl)thiazol-4-yl)-2-fluorophenyl)propane-1-sulfonamide, N-(3-(5-(2-aminopyrimidin-4-yl)-2-(1-((1-(((1S,4r)-4-(4-((S)-2,6-dioxopiperidin-3-yl)phenoxy)cyclohexyl)methyl)piperidin-4-yl)methyl)piperidin-4-yl)thiazol-4-yl)-2-fluorophenyl)propane-1-sulfonamide, (R)—N-(3-(5-(2-aminopyrimidin-4-yl)-2-(2-(1-(2-(2,6-dioxopiperidin-3-yl)-1,3-dioxoisoindolin-5-yl)piperidine-4-carbonyl)-2-azaspiro[3.5]nonan-7-yl)thiazol-4-yl)-2-fluorophenyl)propane-1-sulfonamide, (S)—N-(3-(2-(1-(1-(2-(1-(4-((2,6-dioxopiperidin-3-yl)amino)phenyl)piperidin-4-yl)acetyl)piperidine-4-carbonyl)piperidin-4-yl)-5-(pyrimidin-4-yl)thiazol-4-yl)-2-fluorophenyl)propane-1-sulfonamide, (S)—N-(3-(5-(2-aminopyrimidin-4-yl)-2-(2-(1-(1-(1-(5-(2,6-dioxopiperidin-3-yl)pyridin-2-yl)piperidine-4-carbonyl)piperidine-4-carbonyl)piperidin-4-yl)ethyl)thiazol-4-yl)-2-fluorophenyl)propane-1-sulfonamide, (R)—N-(3-(5-(2-aminopyrimidin-4-yl)-2-(2-(2-(4-(2-(2,6-dioxopiperidin-3-yl)-1,3-dioxoisoindolin-5-yl)piperazin-1-yl)acetyl)-2-azaspiro[3.5]nonan-7-yl)thiazol-4-yl)-2-fluorophenyl)propane-1-sulfonamide, (R)—N-(3-(5-(2-aminopyrimidin-4-yl)-2-(4-(4-(1-(5-(2,6-dioxopiperidin-3-yl)pyridin-2-yl)piperidine-4-carbonyl)piperazin-1-yl)phenyl)thiazol-4-yl)-2-fluorophenyl)propane-1-sulfonamide, (S)—N-(3-(2-(1-(1-(1-(5-(2,6-dioxopiperidin-3-yl)pyridin-2-yl)piperidine-4-carbonyl)piperidine-4-carbonyl)piperidin-4-yl)-5-(pyrimidin-4-yl)thiazol-4-yl)-2-fluorophenyl)propane-1-sulfonamide, (R)—N-(3-(5-(2-aminopyrimidin-4-yl)-2-(1-(1-(1-(4-(2,6-dioxopiperidin-3-yl)phenyl)piperidine-4-carbonyl)piperidine-4-carbonyl)piperidin-4-yl)thiazol-4-yl)-2-fluorophenyl)propane-1-sulfonamide, (R)—N-(3-(5-(2-aminopyrimidin-4-yl)-2-(4-(4-((1-(4-(2,6-dioxopiperidin-3-yl)phenyl)piperidin-4-yl)methyl)piperazin-1-yl)phenyl)thiazol-4-yl)-2-fluorophenyl)propane-1-sulfonamide, (S)—N-(3-(5-(2-aminopyrimidin-4-yl)-2-(1-((1-(1-(5-(2,6-dioxopiperidin-3-yl)pyridin-2-yl)piperidine-4-carbonyl)piperidin-4-yl)methyl)piperidin-4-yl)thiazol-4-yl)-2-fluorophenyl)propane-1-sulfonamide, (R)—N-(3-(5-(2-aminopyrimidin-4-yl)-2-(4-(4-(2-(1-(5-(2,6-dioxopiperidin-3-yl)pyridin-2-yl)piperidin-4-yl)acetyl)piperazin-1-yl)phenyl)thiazol-4-yl)-2-fluorophenyl)propane-1-sulfonamide, (R)—N-(3-(5-(2-aminopyrimidin-4-yl)-2-(3-(2-(1′-(2-(2,6-dioxopiperidin-3-yl)-1,3-dioxoisoindolin-5-yl)-[1,4′-bipiperidin]-4-yl)acetyl)-3-azaspiro[5.5]undecan-9-yl)thiazol-4-yl)-2-fluorophenyl)propane-1-sulfonamide, (S)—N-(3-(5-(2-aminopyrimidin-4-yl)-2-(1-(1-((1-(4-(2,6-dioxopiperidin-3-yl)phenyl)piperidin-4-yl)methyl)piperidine-4-carbonyl)-4-methylpiperidin-4-yl)thiazol-4-yl)-2-fluorophenyl)propane-1-sulfonamide, (S)—N-(3-(2-(1-(1-(1-(4-(2,6-dioxopiperidin-3-yl)phenyl)piperidine-4-carbonyl)-4-methylpiperidine-4-carbonyl)piperidin-4-yl)-5-(2-(isopropylamino)pyrimidin-4-yl)thiazol-4-yl)-2-fluorophenyl)propane-1-sulfonamide, (R)—N-(3-(5-(2-aminopyrimidin-4-yl)-2-(3-(2-(4-(2-(2,6-dioxopiperidin-3-yl)-1,3-dioxoisoindolin-5-yl)piperazin-1-yl)acetyl)-3-azaspiro[5.5]undecan-9-yl)thiazol-4-yl)-2-fluorophenyl)propane-1-sulfonamide, N-(3-(5-(2-aminopyrimidin-4-yl)-2-(1-((1-((1R,4r)-4-(4-((R)-2,6-dioxopiperidin-3-yl)phenoxy)cyclohexane-1-carbonyl)piperidin-4-yl)methyl)piperidin-4-yl)thiazol-4-yl)-2-fluorophenyl)propane-1-sulfonamide, (R)—N-(3-(5-(2-aminopyrimidin-4-yl)-2-(2-(6-(2-(2,6-dioxopiperidin-3-yl)-3-oxoisoindolin-5-yl)hexanoyl)-2-azaspiro[3.5]nonan-7-yl)thiazol-4-yl)-2-fluorophenyl)propane-1-sulfonamide, N-(3-(2-(1-(1-(1-(5-((S)-2,6-dioxopiperidin-3-yl)pyridin-2-yl)piperidine-4-carbonyl)piperidine-4-carbonyl)piperidin-4-yl)-5-(2-(((R)-1-hydroxypropan-2-yl)amino)pyrimidin-4-yl)thiazol-4-yl)-2-fluorophenyl)propane-1-sulfonamide, (S)—N-(3-(5-(2-aminopyrimidin-4-yl)-2-(4-(4-((1-(5-(2,6-dioxopiperidin-3-yl)pyridin-2-yl)piperidin-4-yl)methyl)piperazin-1-yl)phenyl)thiazol-4-yl)-2-fluorophenyl)propane-1-sulfonamide, rac-(3R)-3-{6-[4-(4-{4-[5-(2-aminopyrimidin-4-yl)-4-(3-{[ethyl(methyl)sulfamoyl]amino}-2-fluorophenyl)-1,3-thiazol-2-yl]piperidine-1-carbonyl}piperidine-1-carbonyl)piperidin-1-yl]pyridin-3-yl}piperidine-2,6-dione, rac-(3R)-3-{6-[4-(4-{4-[4-(3-{[ethyl(methyl)sulfamoyl]amino}-2-fluorophenyl)-5-{2-[(propan-2-yl)amino]pyrimidin-4-yl}-1,3-thiazol-2-yl]piperidine-1-carbonyl}piperidine-1-carbonyl)piperidin-1-yl]pyridin-3-yl}piperidine-2,6-dione (S)—N-(3-(5-(2-aminopyrimidin-4-yl)-2-(1-(1-((1-(4-(2,6-dioxopiperidin-3-yl)phenyl)piperidin-4-yl)methyl)piperidine-4-carbonyl)piperidin-4-yl)thiazol-4-yl)-2-fluorophenyl)propane-1-sulfonamide, (S)—N-(3-(5-(2-aminopyrimidin-4-yl)-2-(1-(1-((1-(5-(2,6-dioxopiperidin-3-yl)pyridin-2-yl)piperidin-4-yl)methyl)piperidine-4-carbonyl)piperidin-4-yl)thiazol-4-yl)-2-fluorophenyl)propane-1-sulfonamide, (R)—N-(3-(5-(2-aminopyrimidin-4-yl)-2-(3-((1-(2-(2,6-dioxopiperidin-3-yl)-1-oxoisoindolin-5-yl)piperidin-4-yl)methyl)-3-azaspiro[5.5]undecan-9-yl)thiazol-4-yl)-2-fluorophenyl)propane-1-sulfonamide, (R)—N-(3-(5-(2-aminopyrimidin-4-yl)-2-(1-(2-(2-(2-(2,6-dioxopiperidin-3-yl)-1,3-dioxoisoindolin-5-yl)-2,7-diazaspiro[3.5]nonan-7-yl)acetyl)-4-methylpiperidin-4-yl)thiazol-4-yl)-2-fluorophenyl)propane-1-sulfonamide, (R)—N-(3-(5-(2-aminopyrimidin-4-yl)-2-(3-(1-(2-(2,6-dioxopiperidin-3-yl)-1,3-dioxoisoindolin-5-yl)piperidine-4-carbonyl)-3-azaspiro[5.5]undecan-9-yl)thiazol-4-yl)-2-fluorophenyl)propane-1-sulfonamide, (R)—N-(3-(2-(1-(2-(2-(2-(2,6-dioxopiperidin-3-yl)-1,3-dioxoisoindolin-5-yl)-2,7-diazaspiro[3.5]nonan-7-yl)acetyl)piperidin-4-yl)-5-(2-(methylamino)pyrimidin-4-yl)thiazol-4-yl)-2-fluorophenyl)propane-1-sulfonamide, (R)—N-(3-(5-(2-aminopyrimidin-4-yl)-2-(3-(6-(2-(2,6-dioxopiperidin-3-yl)-3-oxoisoindolin-5-yl)hexanoyl)-3-azaspiro[5.5]undecan-9-yl)thiazol-4-yl)-2-fluorophenyl)propane-1-sulfonamide, (R)—N-(3-(5-(2-aminopyrimidin-4-yl)-2-(1-(1-(2-(1-(5-(2,6-dioxopiperidin-3-yl)pyridin-2-yl)piperidin-4-yl)acetyl)-4-methylpiperidine-4-carbonyl)piperidin-4-yl)thiazol-4-yl)-2-fluorophenyl)propane-1-sulfonamide, (S)—N-(3-(2-(1-(1-(1-(5-(2,6-dioxopiperidin-3-yl)pyridin-2-yl)piperidine-4-carbonyl)piperidine-4-carbonyl)piperidin-4-yl)-5-(2-(methylamino)pyrimidin-4-yl)thiazol-4-yl)-2-fluorophenyl)propane-1-sulfonamide, (R)—N-(3-(5-(2-aminopyrimidin-4-yl)-2-(1-(1-(2-(1-(5-(2,6-dioxopiperidin-3-yl)pyridin-2-yl)piperidin-4-yl)acetyl)piperidine-4-carbonyl)piperidin-4-yl)thiazol-4-yl)-2-fluorophenyl)propane-1-sulfonamide, (S)—N-(3-(2-(1-(1-(1-(5-(2,6-dioxopiperidin-3-yl)pyridin-2-yl)piperidine-4-carbonyl)piperidine-4-carbonyl)piperidin-4-yl)-5-(2-(isopropylamino)pyrimidin-4-yl)thiazol-4-yl)-2-fluorophenyl)propane-1-sulfonamide, (S)—N-(3-(5-(2-(cyclopropylamino)pyrimidin-4-yl)-2-(1-(1-(1-(5-(2,6-dioxopiperidin-3-yl)pyridin-2-yl)piperidine-4-carbonyl)piperidine-4-carbonyl)piperidin-4-yl)thiazol-4-yl)-2-fluorophenyl)propane-1-sulfonamide, (S)—N-(3-(2-(1-(1-(1-(4-(2,6-dioxopiperidin-3-yl)phenyl)piperidine-4-carbonyl)-4-methylpiperidine-4-carbonyl)piperidin-4-yl)-5-(pyrimidin-4-yl)thiazol-4-yl)-2-fluorophenyl)propane-1-sulfonamide, N-(3-(5-(2-aminopyrimidin-4-yl)-2-(3-(2-((R)-1-(4-((S)-2,6-dioxopiperidin-3-yl)phenyl)pyrrolidin-3-yl)acetyl)-3-azaspiro[5.5]undecan-9-yl)thiazol-4-yl)-2-fluorophenyl)propane-1-sulfonamide, (R)—N-(3-(5-(2-aminopyrimidin-4-yl)-2-(2-(2-(2-(2-(2,6-dioxopiperidin-3-yl)-1,3-dioxoisoindolin-5-yl)-2,7-diazaspiro[3.5]nonan-7-yl)acetyl)-2-azaspiro[3.5]nonan-7-yl)thiazol-4-yl)-2-fluorophenyl)propane-1-sulfonamide, (R)—N-(3-(5-(2-aminopyrimidin-4-yl)-2-(1-((1-(2-(1-(5-(2,6-dioxopiperidin-3-yl)pyridin-2-yl)piperidin-4-yl)acetyl)piperidin-4-yl)methyl)piperidin-4-yl)thiazol-4-yl)-2-fluorophenyl)propane-1-sulfonamide, (R)—N-(3-(5-(2-aminopyrimidin-4-yl)-2-(1-(2-(2-(2-(2,6-dioxopiperidin-3-yl)-1,3-dioxoisoindolin-5-yl)-2,7-diazaspiro[3.5]nonan-7-yl)acetyl)piperidin-4-yl)thiazol-4-yl)-2-fluorophenyl)propane-1-sulfonamide, (R)—N-(3-(5-(2-aminopyrimidin-4-yl)-2-(3-(1-(1-(2,6-dioxopiperidin-3-yl)-3-methyl-2-oxo-2,3-dihydro-1H-benzo[d]imidazol-4-yl)piperidine-4-carbonyl)-3-azaspiro[5.5]undecan-9-yl)thiazol-4-yl)-2-fluorophenyl)propane-1-sulfonamide, (R)—N-(3-(2-(1-(1-((1-(4-(2,6-dioxopiperidin-3-yl)phenyl)piperidin-4-yl)methyl)piperidine-4-carbonyl)piperidin-4-yl)-5-(2-(methylamino)pyrimidin-4-yl)thiazol-4-yl)-2-fluorophenyl)propane-1-sulfonamide, (R)—N-(3-(5-(2-aminopyrimidin-4-yl)-2-(1-(1-(1-(2-(2,6-dioxopiperidin-3-yl)-1,3-dioxoisoindolin-5-yl)piperidin-4-yl)azetidine-3-carbonyl)piperidin-4-yl)thiazol-4-yl)-2-fluorophenyl)propane-1-sulfonamide, (R)—N-(3-(5-(2-aminopyrimidin-4-yl)-2-(2-(1-(1-(2-(2,6-dioxopiperidin-3-yl)-1,3-dioxoisoindolin-5-yl)piperidin-4-yl)azetidine-3-carbonyl)-2-azaspiro[3.5]nonan-7-yl)thiazol-4-yl)-2-fluorophenyl)propane-1-sulfonamide, (S)—N-(3-(5-(2-aminopyrimidin-4-yl)-2-(1-(1-((1-(1-(2,6-dioxopiperidin-3-yl)-3-methyl-2-oxo-2,3-dihydro-1H-benzo[d]imidazol-4-yl)piperidin-4-yl)methyl)piperidine-4-carbonyl)piperidin-4-yl)thiazol-4-yl)-2-fluorophenyl)propane-1-sulfonamide, (R)—N-(3-(5-(2-aminopyrimidin-4-yl)-2-(3-(2-(2-(2-(2,6-dioxopiperidin-3-yl)-1,3-dioxoisoindolin-5-yl)-2,7-diazaspiro[3.5]nonan-7-yl)acetyl)-3-azaspiro[5.5]undecan-9-yl)thiazol-4-yl)-2-fluorophenyl)propane-1-sulfonamide, (R)—N-(3-(5-(2-aminopyrimidin-4-yl)-2-(2-(1-(2-(2-(2-(2,6-dioxopiperidin-3-yl)-1,3-dioxoisoindolin-5-yl)-2,7-diazaspiro[3.5]nonan-7-yl)acetyl)piperidin-4-yl)propan-2-yl)thiazol-4-yl)-2-fluorophenyl)propane-1-sulfonamide, and (R)-4-((4-(5-(2-aminopyrimidin-4-yl)-4-(2-fluoro-3-(propylsulfonamido)phenyl)thiazol-2-yl)piperidin-1-yl)methyl)-N-(2,6-dioxopiperidin-3-yl)benzamide. In certain embodiments, the compound of Formula (I) is selected from the group consisting of (R)-4-(4-((6-((4-(2-(tert-butyl)-4-(2-fluoro-3-(propylsulfonamido)phenyl)thiazol-5-yl)pyrimidin-2-yl)amino)-2-azaspiro[3.3]heptan-2-yl)methyl)piperidin-1-yl)-N-(2,6-dioxopiperidin-3-yl)benzamide, N-(3-(2-(tert-butyl)-5-(2-((2-(2-((S)-1-(5-((S)-2,6-dioxopiperidin-3-yl)pyridin-2-yl)pyrrolidin-3-yl)ethyl)-2-azaspiro[3.3]heptan-6-yl)amino)pyrimidin-4-yl)thiazol-4-yl)-2-fluorophenyl)propane-1-sulfonamide, (R)—N-(3-(2-(tert-butyl)-5-(2-((2-(2-(1′-(2-(2,6-dioxopiperidin-3-yl)-1,3-dioxoisoindolin-5-yl)-[1,4′-bipiperidin]-4-yl)acetyl)-2-azaspiro[3.3]heptan-6-yl)amino)pyrimidin-4-yl)thiazol-4-yl)-2-fluorophenyl)propane-1-sulfonamide, (R)—N-(3-(2-(tert-butyl)-5-(2-((2-(1-(1-(2,6-dioxopiperidin-3-yl)-3-methyl-2-oxo-2,3-dihydro-1H-benzo[d]imidazol-4-yl)piperidine-4-carbonyl)-2-azaspiro[3.3]heptan-6-yl)amino)pyrimidin-4-yl)thiazol-4-yl)-2-fluorophenyl)propane-1-sulfonamide, (R)—N-(3-(2-(tert-butyl)-5-(2-((2-(1-(1-(2-(2,6-dioxopiperidin-3-yl)-1,3-dioxoisoindolin-5-yl)piperidin-4-yl)azetidine-3-carbonyl)-2-azaspiro[3.3]heptan-6-yl)amino)pyrimidin-4-yl)thiazol-4-yl)-2-fluorophenyl)propane-1-sulfonamide, (R)—N-(3-(2-(tert-butyl)-5-(2-((2-(2-(2-(2-(2,6-dioxopiperidin-3-yl)-1,3-dioxoisoindolin-5-yl)-2,7-diazaspiro[3.5]nonan-7-yl)acetyl)-2-azaspiro[3.3]heptan-6-yl)amino)pyrimidin-4-yl)thiazol-4-yl)-2-fluorophenyl)propane-1-sulfonamide, (R)—N-(3-(2-(tert-butyl)-5-(2-((2-(6-(2-(2,6-dioxopiperidin-3-yl)-3-oxoisoindolin-5-yl)hexanoyl)-2-azaspiro[3.3]heptan-6-yl)amino)pyrimidin-4-yl)thiazol-4-yl)-2-fluorophenyl)propane-1-sulfonamide, (R)—N-(3-(2-(tert-butyl)-5-(2-((2-((1-((1-(4-(2,6-dioxopiperidin-3-yl)phenyl)piperidin-4-yl)methyl)piperidin-4-yl)methyl)-2-azaspiro[3.3]heptan-6-yl)amino)pyrimidin-4-yl)thiazol-4-yl)-2-fluorophenyl)propane-1-sulfonamide, (R)—N-(3-(2-(tert-butyl)-5-(2-((2-(2-(4-(3-(2-(2,6-dioxopiperidin-3-yl)-1,3-dioxoisoindolin-4-yl)propyl)piperazin-1-yl)acetyl)-2-azaspiro[3.3]heptan-6-yl)amino)pyrimidin-4-yl)thiazol-4-yl)-2-fluorophenyl)propane-1-sulfonamide, (R)—N-(3-(2-(tert-butyl)-5-(2-((2-(1-((1-(4-(2,6-dioxopiperidin-3-yl)phenyl)piperidin-4-yl)methyl)-4-fluoropiperidine-4-carbonyl)-2-azaspiro[3.3]heptan-6-yl)amino)pyrimidin-4-yl)thiazol-4-yl)-2-fluorophenyl)propane-1-sulfonamide, (R)—N-(3-(2-(tert-butyl)-5-(2-((2-((1-(1-(4-(2,6-dioxopiperidin-3-yl)phenyl)piperidine-4-carbonyl)piperidin-4-yl)methyl)-2-azaspiro[3.3]heptan-6-yl)amino)pyrimidin-4-yl)thiazol-4-yl)-2-fluorophenyl)propane-1-sulfonamide, (S)—N-(3-(2-(tert-butyl)-5-(2-((1′-((1-(5-(2,6-dioxopiperidin-3-yl)pyridin-2-yl)piperidin-4-yl)methyl)-[1,4′-bipiperidin]-4-yl)amino)pyrimidin-4-yl)thiazol-4-yl)-2-fluorophenyl)propane-1-sulfonamide, (R)—N-(3-(2-(tert-butyl)-5-(2-((7-(2-(4-(4-((2,6-dioxopiperidin-3-yl)amino)phenyl)piperidin-1-yl)acetyl)-7-azaspiro[3.5]nonan-2-yl)amino)pyrimidin-4-yl)thiazol-4-yl)-2-fluorophenyl)propane-1-sulfonamide, N-(3-(2-(tert-butyl)-5-(2-((7-(((1S,4r)-4-(4-((S)-2,6-dioxopiperidin-3-yl)phenoxy)cyclohexyl)methyl)-7-azaspiro[3.5]nonan-2-yl)amino)pyrimidin-4-yl)thiazol-4-yl)-2-fluorophenyl)propane-1-sulfonamide, (R)—N-(3-(2-(tert-butyl)-5-(2-((7-((1-(2-(2,6-dioxopiperidin-3-yl)-1-oxo-1,2-dihydroisoquinolin-6-yl)piperidin-4-yl)methyl)-7-azaspiro[3.5]nonan-2-yl)amino)pyrimidin-4-yl)thiazol-4-yl)-2-fluorophenyl)propane-1-sulfonamide, (R)—N-(3-(2-(tert-butyl)-5-(2-((7-((1-(4-(2,6-dioxopiperidin-3-yl)phenyl)piperidin-4-yl)methyl)-7-azaspiro[3.5]nonan-2-yl)amino)pyrimidin-4-yl)thiazol-4-yl)-2-fluorophenyl)propane-1-sulfonamide, and (R)—N-(3-(2-(tert-butyl)-5-(2-((7-(2-(4-(2-(2,6-dioxopiperidin-3-yl)-1,3-dioxoisoindolin-5-yl)piperazin-1-yl)acetyl)-7-azaspiro[3.5]nonan-2-yl)amino)pyrimidin-4-yl)thiazol-4-yl)-2-fluorophenyl)propane-1-sulfonamide. In certain embodiments, the compound of Formula (I) is selected from the group consisting of (S)—N-(3-(2-(tert-butyl)-5-(2-((4-(1-((1-(5-(2,6-dioxopiperidin-3-yl)pyridin-3-yl)piperidin-4-yl)methyl)piperidin-4-yl)phenyl)amino)pyrimidin-4-yl)thiazol-4-yl)-2-fluorophenyl)propane-1-sulfonamide, (R)—N-(3-(2-(tert-butyl)-5-(2-((4-(1-(2-((2-(2,6-dioxopiperidin-3-yl)-1,3-dioxoisoindolin-5-yl)oxy)acetyl)piperidin-4-yl)phenyl)amino)pyrimidin-4-yl)thiazol-4-yl)-2-fluorophenyl)propane-1-sulfonamide, (R)—N-(3-(2-(tert-butyl)-5-(2-((4-(1-(2-((2-(2,6-dioxopiperidin-3-yl)-1,3-dioxoisoindolin-5-yl)oxy)acetyl)piperidin-4-yl)phenyl)amino)pyrimidin-4-yl)thiazol-4-yl)-2-fluorophenyl)propane-1-sulfonamide, (R)—N-(3-(2-(tert-butyl)-5-(2-((4-(1-(2-(4-(4-(2,6-dioxopiperidin-3-yl)pyridin-2-yl)piperazin-1-yl)acetyl)piperidin-4-yl)phenyl)amino)pyrimidin-4-yl)thiazol-4-yl)-2-fluorophenyl)propane-1-sulfonamide, N-(3-(2-(tert-butyl)-5-(2-((4-(1-(2-((S)-1-(5-((S)-2,6-dioxopiperidin-3-yl)pyridin-2-yl)pyrrolidin-3-yl)ethyl)piperidin-4-yl)phenyl)amino)pyrimidin-4-yl)thiazol-4-yl)-2-fluorophenyl)propane-1-sulfonamide, (R)-4-(4-((4-(4-((4-(2-(tert-butyl)-4-(2-fluoro-3-(propylsulfonamido)phenyl)thiazol-5-yl)pyrimidin-2-yl)amino)phenyl)piperidin-1-yl)methyl)piperidin-1-yl)-N-(2,6-dioxopiperidin-3-yl)benzamide, (R)-5-(4-((4-(4-((4-(2-(tert-butyl)-4-(2-fluoro-3-(propylsulfonamido)phenyl)thiazol-5-yl)pyrimidin-2-yl)amino)phenyl)piperidin-1-yl)methyl)piperidin-1-yl)-N-(2,6-dioxopiperidin-3-yl)picolinamide, N-(3-(2-(tert-butyl)-5-(2-((4-(1-(((1S,4r)-4-((5-((S)-2,6-dioxopiperidin-3-yl)pyridin-2-yl)oxy)cyclohexyl)methyl)piperidin-4-yl)phenyl)amino)pyrimidin-4-yl)thiazol-4-yl)-2-fluorophenyl)propane-1-sulfonamide, (S)—N-(3-(2-(tert-butyl)-5-(2-((4-(1-((1-(4-(2,6-dioxopiperidin-3-yl)phenyl)piperidin-4-yl)methyl)piperidin-4-yl)phenyl)amino)pyrimidin-4-yl)thiazol-4-yl)-2-fluorophenyl)propane-1-sulfonamide, N-(3-(2-(tert-butyl)-5-(2-((4-(1-(2-((S)-1-(4-((S)-2,6-dioxopiperidin-3-yl)phenyl)pyrrolidin-3-yl)acetyl)piperidin-4-yl)phenyl)amino)pyrimidin-4-yl)thiazol-4-yl)-2-fluorophenyl)propane-1-sulfonamide, N-(3-(2-(tert-butyl)-5-(2-((4-(1-(2-((R)-1-(4-((S)-2,6-dioxopiperidin-3-yl)phenyl)pyrrolidin-3-yl)acetyl)piperidin-4-yl)phenyl)amino)pyrimidin-4-yl)thiazol-4-yl)-2-fluorophenyl)propane-1-sulfonamide, N-(3-(2-(tert-butyl)-5-(2-((4-(1-((R)-1-(5-((S)-2,6-dioxopiperidin-3-yl)pyridin-2-yl)pyrrolidine-3-carbonyl)piperidin-4-yl)phenyl)amino)pyrimidin-4-yl)thiazol-4-yl)-2-fluorophenyl)propane-1-sulfonamide, (S)—N-(3-(2-(tert-butyl)-5-(2-((4-(1-(1-(4-(2,6-dioxopiperidin-3-yl)pyridin-2-yl)piperidine-4-carbonyl)piperidin-4-yl)phenyl)amino)pyrimidin-4-yl)thiazol-4-yl)-2-fluorophenyl)propane-1-sulfonamide, (R)—N-(3-(2-(tert-butyl)-5-(2-((4-(1-(2-(1′-(2-(2,6-dioxopiperidin-3-yl)-1,3-dioxoisoindolin-5-yl)-[1,4′-bipiperidin]-4-yl)acetyl)piperidin-4-yl)phenyl)amino)pyrimidin-4-yl)thiazol-4-yl)-2-fluorophenyl)propane-1-sulfonamide, (S)—N-(3-(2-(tert-butyl)-5-(2-((4-(1-(1-(5-(2,6-dioxopiperidin-3-yl)pyridin-2-yl)piperidine-4-carbonyl)piperidin-4-yl)phenyl)amino)pyrimidin-4-yl)thiazol-4-yl)-2-fluorophenyl)propane-1-sulfonamide, N-(3-(2-(tert-butyl)-5-(2-((4-(1-(2-((S)-1-(1-((R)-2,6-dioxopiperidin-3-yl)-3-methyl-2-oxo-2,3-dihydro-1H-benzo[d]imidazol-5-yl)pyrrolidin-3-yl)acetyl)piperidin-4-yl)phenyl)amino)pyrimidin-4-yl)thiazol-4-yl)-2-fluorophenyl)propane-1-sulfonamide, (R)—N-(3-(2-(tert-butyl)-5-(2-((4-(1-(1-(1-(2,6-dioxopiperidin-3-yl)-3-methyl-2-oxo-2,3-dihydro-1H-benzo[d]imidazol-4-yl)piperidine-4-carbonyl)piperidin-4-yl)phenyl)amino)pyrimidin-4-yl)thiazol-4-yl)-2-fluorophenyl)propane-1-sulfonamide, (S)—N-(3-(2-(tert-butyl)-5-(2-((4-(1-(1-(5-(2,6-dioxopiperidin-3-yl)pyridin-2-yl)azetidine-3-carbonyl)piperidin-4-yl)phenyl)amino)pyrimidin-4-yl)thiazol-4-yl)-2-fluorophenyl)propane-1-sulfonamide, (R)—N-(3-(2-(tert-butyl)-5-(2-((4-(1-(1-(2-(2,6-dioxopiperidin-3-yl)-1,3-dioxoisoindolin-5-yl)piperidine-4-carbonyl)piperidin-4-yl)phenyl)amino)pyrimidin-4-yl)thiazol-4-yl)-2-fluorophenyl)propane-1-sulfonamide, (R)—N-(3-(2-(tert-butyl)-5-(2-((4-(1-(2-(2-(2-(2,6-dioxopiperidin-3-yl)-1,3-dioxoisoindolin-5-yl)-2,7-diazaspiro[3.5]nonan-7-yl)acetyl)piperidin-4-yl)phenyl)amino)pyrimidin-4-yl)thiazol-4-yl)-2-fluorophenyl)propane-1-sulfonamide, (R)—N-(3-(2-(tert-butyl)-5-(2-((4-(1-(6-(2-(2,6-dioxopiperidin-3-yl)-3-oxoisoindolin-5-yl)hexanoyl)piperidin-4-yl)phenyl)amino)pyrimidin-4-yl)thiazol-4-yl)-2-fluorophenyl)propane-1-sulfonamide, (R)—N-(3-(2-(tert-butyl)-5-(2-((4-(1-(2-(4-(2-(2,6-dioxopiperidin-3-yl)-1,3-dioxoisoindolin-5-yl)piperazin-1-yl)acetyl)piperidin-4-yl)phenyl)amino)pyrimidin-4-yl)thiazol-4-yl)-2-fluorophenyl)propane-1-sulfonamide, (S)-5-(4-((4-(((4-(2-(tert-butyl)-4-(2-fluoro-3-(propylsulfonamido)phenyl)thiazol-5-yl)pyrimidin-2-yl)amino)methyl)piperidin-1-yl)methyl)piperidin-1-yl)-N-(2,6-dioxopiperidin-3-yl)picolinamide, (S)-5-(4-((4-(1-((4-(2-(tert-butyl)-4-(2-fluoro-3-(propylsulfonamido)phenyl)thiazol-5-yl)pyrimidin-2-yl)amino)-2-methylpropan-2-yl)piperidin-1-yl)methyl)piperidin-1-yl)-N-(2,6-dioxopiperidin-3-yl)picolinamide, (R)—N-(3-(2-(tert-butyl)-5-(2-(((1-((1-(4-(2,6-dioxopiperidin-3-yl)phenyl)piperidin-4-yl)methyl)piperidin-4-yl)methyl)amino)pyrimidin-4-yl)thiazol-4-yl)-2-fluorophenyl)propane-1-sulfonamide, (R)—N-(3-(2-(tert-butyl)-5-(2-((2-(1-((1-(4-(2,6-dioxopiperidin-3-yl)phenyl)piperidin-4-yl)methyl)piperidin-4-yl)-2-methylpropyl)amino)pyrimidin-4-yl)thiazol-4-yl)-2-fluorophenyl)propane-1-sulfonamide, 5-(4-(((R)-3-(((4-(2-(tert-butyl)-4-(2-fluoro-3-(propylsulfonamido)phenyl)thiazol-5-yl)pyrimidin-2-yl)amino)methyl)pyrrolidin-1-yl)methyl)piperidin-1-yl)-N—((S)-2,6-dioxopiperidin-3-yl)picolinamide, N-(3-(2-(tert-butyl)-5-(2-(((1r,4r)-4-((4-((1-(4-((S)-2,6-dioxopiperidin-3-yl)phenyl)piperidin-4-yl)methyl)piperazin-1-yl)methyl)cyclohexyl)amino)pyrimidin-4-yl)thiazol-4-yl)-2-fluorophenyl)propane-1-sulfonamide, N-(3-(2-(tert-butyl)-5-(2-(((1s,4s)-4-(4-(1-(5-((S)-2,6-dioxopiperidin-3-yl)pyridin-2-yl)piperidine-4-carbonyl)piperazin-1-yl)cyclohexyl)amino)pyrimidin-4-yl)thiazol-4-yl)-2-fluorophenyl)propane-1-sulfonamide, N-(3-(2-(tert-butyl)-5-(2-(((1r,4r)-4-(4-(1-(5-((S)-2,6-dioxopiperidin-3-yl)pyridin-2-yl)piperidine-4-carbonyl)piperazin-1-yl)cyclohexyl)amino)pyrimidin-4-yl)thiazol-4-yl)-2-fluorophenyl)propane-1-sulfonamide, N-(3-(2-(tert-butyl)-5-(2-(((1r,4r)-4-(4-((1-(5-((S)-2,6-dioxopiperidin-3-yl)pyridin-2-yl)piperidin-4-yl)methyl)piperazin-1-yl)cyclohexyl)amino)pyrimidin-4-yl)thiazol-4-yl)-2-fluorophenyl)propane-1-sulfonamide, N-(3-(2-(tert-butyl)-5-(2-(((1s,4s)-4-(4-((1-(5-((S)-2,6-dioxopiperidin-3-yl)pyridin-2-yl)piperidin-4-yl)methyl)piperazin-1-yl)cyclohexyl)amino)pyrimidin-4-yl)thiazol-4-yl)-2-fluorophenyl)propane-1-sulfonamide, (3R)-3-{4-[4-({4-[6-({4-[2-tert-butyl-4-(3-{[ethyl(methyl)sulfamoyl]amino}-2-fluorophenyl)-1,3-thiazol-5-yl]pyrimidin-2-yl}amino)pyridin-3-yl]piperazin-1-yl}methyl)piperidin-1-yl]phenyl}piperidine-2,6-dione, (3S)-3-{4-[4-({4-[6-({4-[2-tert-butyl-4-(3-{[ethyl(methyl)sulfamoyl]amino}-2-fluorophenyl)-1,3-thiazol-5-yl]pyrimidin-2-yl}amino)pyridin-3-yl]piperazin-1-yl}methyl)piperidin-1-yl]phenyl}piperidine-2,6-dione, rac-(3S)-3-{6-[4-({4-[6-({4-[2-tert-butyl-4-(3-{[ethyl(methyl)sulfamoyl]amino}-2-fluorophenyl)-1,3-thiazol-5-yl]pyrimidin-2-yl}amino)pyridin-3-yl]piperazin-1-yl}methyl)piperidin-1-yl]pyridin-3-yl}piperidine-2,6-dione, rac-(3S)-3-(2-{[(1r,4r)-4-{4-[6-({4-[2-tert-butyl-4-(3-{[ethyl(methyl)sulfamoyl]amino}-2-fluorophenyl)-1,3-thiazol-5-yl]pyrimidin-2-yl}amino)pyridin-3-yl]piperazine-1-carbonyl}cyclohexyl]methyl}-1,2,3,4-tetrahydroisoquinolin-6-yl)piperidine-2,6-dione, rac-(3S)-3-(2-{[(1r,4r)-4-{4-[6-({4-[2-tert-butyl-4-(3-{[ethyl(methyl)sulfamoyl]amino}-2-fluorophenyl)-1,3-thiazol-5-yl]pyrimidin-2-yl}amino)pyridin-3-yl]piperazine-1-carbonyl}cyclohexyl]methyl}-1,2,3,4-tetrahydroisoquinolin-7-yl)piperidine-2,6-dione, (R)—N-(3-(2-(tert-butyl)-5-(2-((2-(2-(4-(4-((2,6-dioxopiperidin-3-yl)amino)phenyl)piperidin-1-yl)acetyl)-1,2,3,4-tetrahydroisoquinolin-6-yl)amino)pyrimidin-4-yl)thiazol-4-yl)-2-fluorophenyl)propane-1-sulfonamide, (R)-5-(4-(6-((4-(2-(tert-butyl)-4-(2-fluoro-3-(propylsulfonamido)phenyl)thiazol-5-yl)pyrimidin-2-yl)amino)-1,2,3,4-tetrahydroisoquinoline-2-carbonyl)piperidin-1-yl)-N-(2,6-dioxopiperidin-3-yl)picolinamide, N-(3-(2-(tert-butyl)-5-(2-((2-((R)-1-(5-((S)-2,6-dioxopiperidin-3-yl)pyridin-2-yl)pyrrolidine-3-carbonyl)-1,2,3,4-tetrahydroisoquinolin-6-yl)amino)pyrimidin-4-yl)thiazol-4-yl)-2-fluorophenyl)propane-1-sulfonamide, (S)—N-(3-(2-(tert-butyl)-5-(2-((2-(2-(4-(4-(2,6-dioxopiperidin-3-yl)pyridin-2-yl)piperazin-1-yl)acetyl)-1,2,3,4-tetrahydroisoquinolin-6-yl)amino)pyrimidin-4-yl)thiazol-4-yl)-2-fluorophenyl)propane-1-sulfonamide, (S)—N-(3-(2-(tert-butyl)-5-(2-((1-((1-(2-(6-(2,6-dioxopiperidin-3-yl)-3,4-dihydroisoquinolin-2(1H)-yl)acetyl)piperidin-4-yl)methyl)-1H-pyrazol-4-yl)amino)pyrimidin-4-yl)thiazol-4-yl)-2-fluorophenyl)propane-1-sulfonamide, N-(3-(2-(tert-butyl)-5-(2-((1-((1-((1S,4r)-4-(4-((S)-2,6-dioxopiperidin-3-yl)phenoxy)cyclohexane-1-carbonyl)piperidin-4-yl)methyl)-1H-pyrazol-4-yl)amino)pyrimidin-4-yl)thiazol-4-yl)-2-fluorophenyl)propane-1-sulfonamide, N-(3-(2-(tert-butyl)-5-(2-((1-((1-((1S,4r)-4-((5-((S)-2,6-dioxopiperidin-3-yl)pyridin-2-yl)oxy)cyclohexane-1-carbonyl)piperidin-4-yl)methyl)-1H-pyrazol-4-yl)amino)pyrimidin-4-yl)thiazol-4-yl)-2-fluorophenyl)propane-1-sulfonamide, (R)—N-(3-(2-(tert-butyl)-5-(2-((1-((1-(2-(4-(4-((2,6-dioxopiperidin-3-yl)amino)phenyl)piperidin-1-yl)acetyl)piperidin-4-yl)methyl)-1H-pyrazol-4-yl)amino)pyrimidin-4-yl)thiazol-4-yl)-2-fluorophenyl)propane-1-sulfonamide, (R)-5-(4-(4-((4-((4-(2-(tert-butyl)-4-(2-fluoro-3-(propylsulfonamido)phenyl)thiazol-5-yl)pyrimidin-2-yl)amino)-1H-pyrazol-1-yl)methyl)piperidine-1-carbonyl)piperidin-1-yl)-N-(2,6-dioxopiperidin-3-yl)picolinamide, N-(3-(2-(tert-butyl)-5-(2-((1-((1-((R)-1-(5-((S)-2,6-dioxopiperidin-3-yl)pyridin-2-yl)pyrrolidine-3-carbonyl)piperidin-4-yl)methyl)-1H-pyrazol-4-yl)amino)pyrimidin-4-yl)thiazol-4-yl)-2-fluorophenyl)propane-1-sulfonamide, (S)—N-(3-(2-(tert-butyl)-5-(2-((1-((1-(2-(4-(4-(2,6-dioxopiperidin-3-yl)pyridin-2-yl)piperazin-1-yl)acetyl)piperidin-4-yl)methyl)-1H-pyrazol-4-yl)amino)pyrimidin-4-yl)thiazol-4-yl)-2-fluorophenyl)propane-1-sulfonamide, N-(3-(2-(tert-butyl)-5-(2-((1-((1-(2-((S)-1-(4-((S)-2,6-dioxopiperidin-3-yl)phenyl)pyrrolidin-3-yl)acetyl)piperidin-4-yl)methyl)-1H-pyrazol-4-yl)amino)pyrimidin-4-yl)thiazol-4-yl)-2-fluorophenyl)propane-1-sulfonamide, N-(3-(2-(tert-butyl)-5-(2-((1-((1-(2-((R)-1-(4-((S)-2,6-dioxopiperidin-3-yl)phenyl)pyrrolidin-3-yl)acetyl)piperidin-4-yl)methyl)-1H-pyrazol-4-yl)amino)pyrimidin-4-yl)thiazol-4-yl)-2-fluorophenyl)propane-1-sulfonamide, N-(3-(2-(tert-butyl)-5-(2-((1-(1-((1S,4r)-4-(4-((S)-2,6-dioxopiperidin-3-yl)phenoxy)cyclohexane-1-carbonyl)piperidin-4-yl)-1H-pyrazol-4-yl)amino)pyrimidin-4-yl)thiazol-4-yl)-2-fluorophenyl)propane-1-sulfonamide, N-(3-(2-(tert-butyl)-5-(2-((1-(1-((1S,4r)-4-((5-((S)-2,6-dioxopiperidin-3-yl)pyridin-2-yl)oxy)cyclohexane-1-carbonyl)piperidin-4-yl)-1H-pyrazol-4-yl)amino)pyrimidin-4-yl)thiazol-4-yl)-2-fluorophenyl)propane-1-sulfonamide, (R)—N-(3-(2-(tert-butyl)-5-(2-((1-(1-(2-(4-(4-((2,6-dioxopiperidin-3-yl)amino)phenyl)piperidin-1-yl)acetyl)piperidin-4-yl)-1H-pyrazol-4-yl)amino)pyrimidin-4-yl)thiazol-4-yl)-2-fluorophenyl)propane-1-sulfonamide, (R)-5-(4-(4-(4-((4-(2-(tert-butyl)-4-(2-fluoro-3-(propylsulfonamido)phenyl)thiazol-5-yl)pyrimidin-2-yl)amino)-1H-pyrazol-1-yl)piperidine-1-carbonyl)piperidin-1-yl)-N-(2,6-dioxopiperidin-3-yl)picolinamide, N-(3-(2-(tert-butyl)-5-(2-((1-(1-((R)-1-(5-((S)-2,6-dioxopiperidin-3-yl)pyridin-2-yl)pyrrolidine-3-carbonyl)piperidin-4-yl)-1H-pyrazol-4-yl)amino)pyrimidin-4-yl)thiazol-4-yl)-2-fluorophenyl)propane-1-sulfonamide, N-(3-(2-(tert-butyl)-5-(2-((1-(1-(2-((S)-1-(4-((S)-2,6-dioxopiperidin-3-yl)phenyl)pyrrolidin-3-yl)acetyl)piperidin-4-yl)-1H-pyrazol-4-yl)amino)pyrimidin-4-yl)thiazol-4-yl)-2-fluorophenyl)propane-1-sulfonamide, (S)—N-(3-(2-(tert-butyl)-5-(2-((3-(1-(2-(6-(2,6-dioxopiperidin-3-yl)-3,4-dihydroisoquinolin-2(1H)-yl)acetyl)piperidin-4-yl)phenyl)amino)pyrimidin-4-yl)thiazol-4-yl)-2-fluorophenyl)propane-1-sulfonamide, (R)—N-(3-(2-(tert-butyl)-5-(2-((3-(1-(2-(4-(4-((2,6-dioxopiperidin-3-yl)amino)phenyl)piperidin-1-yl)acetyl)piperidin-4-yl)phenyl)amino)pyrimidin-4-yl)thiazol-4-yl)-2-fluorophenyl)propane-1-sulfonamide, N-(3-(2-(tert-butyl)-5-(2-((3-(1-((R)-1-(5-((S)-2,6-dioxopiperidin-3-yl)pyridin-2-yl)pyrrolidine-3-carbonyl)piperidin-4-yl)phenyl)amino)pyrimidin-4-yl)thiazol-4-yl)-2-fluorophenyl)propane-1-sulfonamide, (S)—N-(3-(2-(tert-butyl)-5-(2-((3-((1-(2-(6-(2,6-dioxopiperidin-3-yl)-3,4-dihydroisoquinolin-2(1H)-yl)acetyl)piperidin-4-yl)oxy)phenyl)amino)pyrimidin-4-yl)thiazol-4-yl)-2-fluorophenyl)propane-1-sulfonamide, (S)—N-(3-(2-(tert-butyl)-5-(2-((3-((1-(1-(4-(2,6-dioxopiperidin-3-yl)pyridin-2-yl)piperidine-4-carbonyl)piperidin-4-yl)oxy)phenyl)amino)pyrimidin-4-yl)thiazol-4-yl)-2-fluorophenyl)propane-1-sulfonamide, (S)—N-(3-(2-(tert-butyl)-5-(2-((3-((1-(1-(3-(2,6-dioxopiperidin-3-yl)phenyl)piperidine-4-carbonyl)piperidin-4-yl)oxy)phenyl)amino)pyrimidin-4-yl)thiazol-4-yl)-2-fluorophenyl)propane-1-sulfonamide, N-(3-(2-(tert-butyl)-5-(2-((3-((1-((1S,4r)-4-(4-((S)-2,6-dioxopiperidin-3-yl)phenoxy)cyclohexane-1-carbonyl)piperidin-4-yl)oxy)phenyl)amino)pyrimidin-4-yl)thiazol-4-yl)-2-fluorophenyl)propane-1-sulfonamide, N-(3-(2-(tert-butyl)-5-(2-((3-((1-((1S,4r)-4-((5-((S)-2,6-dioxopiperidin-3-yl)pyridin-2-yl)oxy)cyclohexane-1-carbonyl)piperidin-4-yl)oxy)phenyl)amino)pyrimidin-4-yl)thiazol-4-yl)-2-fluorophenyl)propane-1-sulfonamide, (R)—N-(3-(2-(tert-butyl)-5-(2-((3-((1-(2-(4-(4-((2,6-dioxopiperidin-3-yl)amino)phenyl)piperidin-1-yl)acetyl)piperidin-4-yl)oxy)phenyl)amino)pyrimidin-4-yl)thiazol-4-yl)-2-fluorophenyl)propane-1-sulfonamide, (R)-5-(4-(4-(3-((4-(2-(tert-butyl)-4-(2-fluoro-3-(propylsulfonamido)phenyl)thiazol-5-yl)pyrimidin-2-yl)amino)phenoxy)piperidine-1-carbonyl)piperidin-1-yl)-N-(2,6-dioxopiperidin-3-yl)picolinamide, N-(3-(2-(tert-butyl)-5-(2-((3-((1-((R)-1-(5-((S)-2,6-dioxopiperidin-3-yl)pyridin-2-yl)pyrrolidine-3-carbonyl)piperidin-4-yl)oxy)phenyl)amino)pyrimidin-4-yl)thiazol-4-yl)-2-fluorophenyl)propane-1-sulfonamide, (S)—N-(3-(2-(tert-butyl)-5-(2-((3-((1-(2-(4-(4-(2,6-dioxopiperidin-3-yl)pyridin-2-yl)piperazin-1-yl)acetyl)piperidin-4-yl)oxy)phenyl)amino)pyrimidin-4-yl)thiazol-4-yl)-2-fluorophenyl)propane-1-sulfonamide, N-(3-(2-(tert-butyl)-5-(2-((3-((1-(2-((S)-1-(4-((S)-2,6-dioxopiperidin-3-yl)phenyl)pyrrolidin-3-yl)acetyl)piperidin-4-yl)oxy)phenyl)amino)pyrimidin-4-yl)thiazol-4-yl)-2-fluorophenyl)propane-1-sulfonamide, N-(3-(2-(tert-butyl)-5-(2-((3-((1-(2-((R)-1-(4-((S)-2,6-dioxopiperidin-3-yl)phenyl)pyrrolidin-3-yl)acetyl)piperidin-4-yl)oxy)phenyl)amino)pyrimidin-4-yl)thiazol-4-yl)-2-fluorophenyl)propane-1-sulfonamide, (S)—N-(3-(2-(tert-butyl)-5-(2-((4-(1-((1-(4-(2,6-dioxopiperidin-3-yl)phenyl)piperidin-4-yl)methyl)piperidin-4-yl)phenyl)amino)pyrimidin-4-yl)thiazol-4-yl)-2-fluorophenyl)propane-1-sulfonamide, (R)—N-(3-(2-(tert-butyl)-5-(2-((4-(1-((1-(4-(2,6-dioxopiperidin-3-yl)phenyl)piperidin-4-yl)methyl)piperidin-4-yl)phenyl)amino)pyrimidin-4-yl)thiazol-4-yl)-2-fluorophenyl)propane-1-sulfonamide, (S)—N-(3-(2-(tert-butyl)-5-(2-((4-(4-((1-(4-(2,6-dioxopiperidin-3-yl)phenyl)piperidin-4-yl)methyl)piperazin-1-yl)-3-fluorophenyl)amino)pyrimidin-4-yl)thiazol-4-yl)-2-fluorophenyl)propane-1-sulfonamide, (R)—N-(3-(2-(tert-butyl)-5-(2-((4-(4-((1-(4-(2,6-dioxopiperidin-3-yl)phenyl)piperidin-4-yl)methyl)piperazin-1-yl)-3-fluorophenyl)amino)pyrimidin-4-yl)thiazol-4-yl)-2-fluorophenyl)propane-1-sulfonamide, (2S,4R)-1-((S)-2-(5-(4-(4-((4-(2-(tert-butyl)-4-(2-fluoro-3-(propylsulfonamido)phenyl)thiazol-5-yl)pyrimidin-2-yl)amino)phenyl)piperidin-1-yl)-5-oxopentanamido)-3,3-dimethylbutanoyl)-4-hydroxy-N—((S)-1-(4-(4-methylthiazol-5-yl)phenyl)ethyl)pyrrolidine-2-carboxamide, (R)—N-(3-(2-(tert-butyl)-5-(2-((2-((1-(2-(2,6-dioxopiperidin-3-yl)-1-oxo-1,2-dihydroisoquinolin-6-yl)piperidin-4-yl)methyl)-1,2,3,4-tetrahydroisoquinolin-6-yl)amino)pyrimidin-4-yl)thiazol-4-yl)-2-fluorophenyl)propane-1-sulfonamide, N-(3-(2-(tert-butyl)-5-(2-((1-((1-(((1S,4r)-4-(4-((S)-2,6-dioxopiperidin-3-yl)phenoxy)cyclohexyl)methyl)piperidin-4-yl)methyl)-1H-pyrazol-4-yl)amino)pyrimidin-4-yl)thiazol-4-yl)-2-fluorophenyl)propane-1-sulfonamide, (R)—N-(3-(2-(tert-butyl)-5-(2-((1-((1-((1-(2-(2,6-dioxopiperidin-3-yl)-1-oxo-1,2-dihydroisoquinolin-6-yl)piperidin-4-yl)methyl)piperidin-4-yl)methyl)-1H-pyrazol-4-yl)amino)pyrimidin-4-yl)thiazol-4-yl)-2-fluorophenyl)propane-1-sulfonamide, (R)-5-(4-((4-((4-((4-(2-(tert-butyl)-4-(2-fluoro-3-(propylsulfonamido)phenyl)thiazol-5-yl)pyrimidin-2-yl)amino)-1H-pyrazol-1-yl)methyl)piperidin-1-yl)methyl)piperidin-1-yl)-N-(2,6-dioxopiperidin-3-yl)picolinamide, N-(3-(2-(tert-butyl)-5-(2-((1-((1-(2-((R)-1-(5-((S)-2,6-dioxopiperidin-3-yl)pyridin-2-yl)pyrrolidin-3-yl)ethyl)piperidin-4-yl)methyl)-1H-pyrazol-4-yl)amino)pyrimidin-4-yl)thiazol-4-yl)-2-fluorophenyl)propane-1-sulfonamide, (R)—N-(3-(2-(tert-butyl)-5-(2-((1-(1-((1-(2-(2,6-dioxopiperidin-3-yl)-1-oxo-1,2-dihydroisoquinolin-6-yl)piperidin-4-yl)methyl)piperidin-4-yl)-1H-pyrazol-4-yl)amino)pyrimidin-4-yl)thiazol-4-yl)-2-fluorophenyl)propane-1-sulfonamide, (R)-5-(4-((4-(4-((4-(2-(tert-butyl)-4-(2-fluoro-3-(propylsulfonamido)phenyl)thiazol-5-yl)pyrimidin-2-yl)amino)-1H-pyrazol-1-yl)piperidin-1-yl)methyl)piperidin-1-yl)-N-(2,6-dioxopiperidin-3-yl)picolinamide, N-(3-(2-(tert-butyl)-5-(2-((1-(1-(2-((R)-1-(5-((S)-2,6-dioxopiperidin-3-yl)pyridin-2-yl)pyrrolidin-3-yl)ethyl)piperidin-4-yl)-1H-pyrazol-4-yl)amino)pyrimidin-4-yl)thiazol-4-yl)-2-fluorophenyl)propane-1-sulfonamide, N-(3-(2-(tert-butyl)-5-(2-((1-(1-(2-((S)-1-(5-((S)-2,6-dioxopiperidin-3-yl)pyridin-2-yl)pyrrolidin-3-yl)ethyl)piperidin-4-yl)-1H-pyrazol-4-yl)amino)pyrimidin-4-yl)thiazol-4-yl)-2-fluorophenyl)propane-1-sulfonamide, (R)—N-(3-(2-(tert-butyl)-5-(2-((3-(1-((1-(2-(2,6-dioxopiperidin-3-yl)-1-oxo-1,2-dihydroisoquinolin-6-yl)piperidin-4-yl)methyl)piperidin-4-yl)phenyl)amino)pyrimidin-4-yl)thiazol-4-yl)-2-fluorophenyl)propane-1-sulfonamide, N-(3-(2-(tert-butyl)-5-(2-((3-(1-(2-((R)-1-(5-((S)-2,6-dioxopiperidin-3-yl)pyridin-2-yl)pyrrolidin-3-yl)ethyl)piperidin-4-yl)phenyl)amino)pyrimidin-4-yl)thiazol-4-yl)-2-fluorophenyl)propane-1-sulfonamide, N-(3-(2-(tert-butyl)-5-(2-((3-(1-(2-((S)-1-(5-((S)-2,6-dioxopiperidin-3-yl)pyridin-2-yl)pyrrolidin-3-yl)ethyl)piperidin-4-yl)phenyl)amino)pyrimidin-4-yl)thiazol-4-yl)-2-fluorophenyl)propane-1-sulfonamide, (S)—N-(3-(2-(tert-butyl)-5-(2-((3-(1-((1-(5-(2,6-dioxopiperidin-3-yl)pyridin-2-yl)-4-methylpiperidin-4-yl)methyl)piperidin-4-yl)phenyl)amino)pyrimidin-4-yl)thiazol-4-yl)-2-fluorophenyl)propane-1-sulfonamide, N-(3-(2-(tert-butyl)-5-(2-((3-((1-(((1S,4r)-4-(4-((S)-2,6-dioxopiperidin-3-yl)phenoxy)cyclohexyl)methyl)piperidin-4-yl)oxy)phenyl)amino)pyrimidin-4-yl)thiazol-4-yl)-2-fluorophenyl)propane-1-sulfonamide, (R)—N-(3-(2-(tert-butyl)-5-(2-((3-((1-((1-(2-(2,6-dioxopiperidin-3-yl)-1-oxo-1,2-dihydroisoquinolin-6-yl)piperidin-4-yl)methyl)piperidin-4-yl)oxy)phenyl)amino)pyrimidin-4-yl)thiazol-4-yl)-2-fluorophenyl)propane-1-sulfonamide, N-(3-(2-(tert-butyl)-5-(2-((3-((1-(2-((R)-1-(5-((S)-2,6-dioxopiperidin-3-yl)pyridin-2-yl)pyrrolidin-3-yl)ethyl)piperidin-4-yl)oxy)phenyl)amino)pyrimidin-4-yl)thiazol-4-yl)-2-fluorophenyl)propane-1-sulfonamide, N-(3-(2-(tert-butyl)-5-(2-((3-((1-(2-((S)-1-(5-((S)-2,6-dioxopiperidin-3-yl)pyridin-2-yl)pyrrolidin-3-yl)ethyl)piperidin-4-yl)oxy)phenyl)amino)pyrimidin-4-yl)thiazol-4-yl)-2-fluorophenyl)propane-1-sulfonamide, (S)—N-(3-(2-(tert-butyl)-5-(2-((3-((1-((1-(5-(2,6-dioxopiperidin-3-yl)pyridin-2-yl)-4-methylpiperidin-4-yl)methyl)piperidin-4-yl)oxy)phenyl)amino)pyrimidin-4-yl)thiazol-4-yl)-2-fluorophenyl)propane-1-sulfonamide, (R)-5-(4-((4-(4-((4-(2-(tert-butyl)-4-(2-fluoro-3-(propylsulfonamido)phenyl)thiazol-5-yl)pyrimidin-2-yl)amino)-1H-pyrazol-1-yl)piperidin-1-yl)methyl)piperidin-1-yl)-N-(2,6-dioxopiperidin-3-yl)picolinamide, (R)-5-(4-((4-((4-((4-(2-(tert-butyl)-4-(2-fluoro-3-(propylsulfonamido)phenyl)thiazol-5-yl)pyrimidin-2-yl)amino)-1H-pyrazol-1-yl)methyl)piperidin-1-yl)methyl)piperidin-1-yl)-N-(2,6-dioxopiperidin-3-yl)picolinamide, (S)—N-(3-(2-(tert-butyl)-5-(2-((4-(4-(1-(4-(2,6-dioxopiperidin-3-yl)phenyl)piperidine-4-carbonyl)piperazin-1-yl)-3-fluorophenyl)amino)pyrimidin-4-yl)thiazol-4-yl)-2-fluorophenyl)propane-1-sulfonamide, (S)—N-(3-(5-(2-((4-(1-((1-(4-(2,6-dioxopiperidin-3-yl)phenyl)piperidin-4-yl)methyl)piperidin-4-yl)phenyl)amino)pyrimidin-4-yl)-2-methylthiazol-4-yl)-2-fluorophenyl)propane-1-sulfonamide, (R)—N-(3-(5-(2-((4-(1-((1-(4-(2,6-dioxopiperidin-3-yl)phenyl)piperidin-4-yl)methyl)piperidin-4-yl)phenyl)amino)pyrimidin-4-yl)-2-methylthiazol-4-yl)-2-fluorophenyl)propane-1-sulfonamide, (S)—N-(3-(5-(2-((5-(4-((1-(5-(2,6-dioxopiperidin-3-yl)pyridin-2-yl)piperidin-4-yl)methyl)piperazin-1-yl)pyridin-2-yl)amino)pyrimidin-4-yl)-2-methylthiazol-4-yl)-2-fluorophenyl)propane-1-sulfonamide, (S)—N-(3-(5-(2-((5-(4-((1-(4-(2,6-dioxopiperidin-3-yl)phenyl)piperidin-4-yl)methyl)piperazin-1-yl)pyridin-2-yl)amino)pyrimidin-4-yl)-2-methylthiazol-4-yl)-2-fluorophenyl)propane-1-sulfonamide, (R)—N-(3-(5-(2-((5-(4-((1-(4-(2,6-dioxopiperidin-3-yl)phenyl)piperidin-4-yl)methyl)piperazin-1-yl)pyridin-2-yl)amino)pyrimidin-4-yl)-2-methylthiazol-4-yl)-2-fluorophenyl)propane-1-sulfonamide, (R)—N-(3-(2-(tert-butyl)-5-(2-((4-(4-((1-(4-(2,6-dioxopiperidin-3-yl)phenyl)piperidin-4-yl)methyl)piperazin-1-yl)phenyl)amino)pyrimidin-4-yl)thiazol-4-yl)-2-fluorophenyl)propane-1-sulfonamide, (S)—N-(3-(2-(tert-butyl)-5-(2-((4-(4-((1-(4-(2,6-dioxopiperidin-3-yl)phenyl)piperidin-4-yl)methyl)piperazin-1-yl)phenyl)amino)pyrimidin-4-yl)thiazol-4-yl)-2-fluorophenyl)propane-1-sulfonamide, (S)—N-(3-(2-(tert-butyl)-5-(2-((4-(4-(1-(5-(2,6-dioxopiperidin-3-yl)pyridin-2-yl)piperidine-4-carbonyl)piperazin-1-yl)-3-fluorophenyl)amino)pyrimidin-4-yl)thiazol-4-yl)-2-fluorophenyl)propane-1-sulfonamide, rac-(3R)-3-[6-(4-{4-[6-({4-[2-tert-butyl-4-(3-{[ethyl(methyl)sulfamoyl]amino}-2-fluorophenyl)-1,3-thiazol-5-yl]pyrimidin-2-yl}amino)pyridin-3-yl]piperazine-1-carbonyl}piperidin-1-yl)pyridin-3-yl]piperidine-2,6-dione, (S)—N-(3-(5-(2-((4-(1-(1-(5-(2,6-dioxopiperidin-3-yl)pyridin-2-yl)piperidine-4-carbonyl)piperidin-4-yl)phenyl)amino)pyrimidin-4-yl)-2-methylthiazol-4-yl)-2-fluorophenyl)propane-1-sulfonamide, N-(3-(2-(tert-butyl)-5-(2-((4-(4-((R)-1-(5-((R)-2,6-dioxopiperidin-3-yl)pyridin-2-yl)pyrrolidine-3-carbonyl)piperazin-1-yl)-3-fluorophenyl)amino)pyrimidin-4-yl)thiazol-4-yl)-2-fluorophenyl)propane-1-sulfonamide, (R)—N-(3-(5-(2-((4-(1-((1-(5-(2,6-dioxopiperidin-3-yl)pyridin-2-yl)piperidin-4-yl)methyl)piperidin-4-yl)phenyl)amino)pyrimidin-4-yl)-2-methylthiazol-4-yl)-2-fluorophenyl)propane-1-sulfonamide, N-(3-(2-(tert-butyl)-5-(2-((4-(4-((R)-1-(5-((R)-2,6-dioxopiperidin-3-yl)pyridin-2-yl)pyrrolidine-3-carbonyl)piperazin-1-yl)-3-fluorophenyl)amino)pyrimidin-4-yl)thiazol-4-yl)-2-fluorophenyl)propane-1-sulfonamide, rac-(3S)-3-[6-(3-{4-[6-({4-[2-tert-butyl-4-(3-{[ethyl(methyl)sulfamoyl]amino}-2-fluorophenyl)-1,3-thiazol-5-yl]pyrimidin-2-yl}amino)pyridin-3-yl]piperazine-1-carbonyl}azetidin-1-yl)pyridin-3-yl]piperidine-2,6-dione, (S)—N-(3-(2-(tert-butyl)-5-(2-((4-(4-(2-(1-(4-((2,6-dioxopiperidin-3-yl)amino)-2-fluorophenyl)-4-hydroxypiperidin-4-yl)acetyl)piperazin-1-yl)-3-fluorophenyl)amino)pyrimidin-4-yl)thiazol-4-yl)-2-fluorophenyl)propane-1-sulfonamide, (R)—N-(3-(2-(tert-butyl)-5-(2-((4-(4-(2-(6-(2,6-dioxopiperidin-3-yl)-3,4-dihydroisoquinolin-2(1H)-yl)acetyl)piperazin-1-yl)-3-fluorophenyl)amino)pyrimidin-4-yl)thiazol-4-yl)-2-fluorophenyl)propane-1-sulfonamide, N-(3-(2-(tert-butyl)-5-(2-((4-(4-((1S,4r)-4-((5-((S)-2,6-dioxopiperidin-3-yl)pyridin-2-yl)oxy)cyclohexane-1-carbonyl)piperazin-1-yl)-3-fluorophenyl)amino)pyrimidin-4-yl)thiazol-4-yl)-2-fluorophenyl)propane-1-sulfonamide, (R)—N-(3-(2-(tert-butyl)-5-(2-((4-(4-(2-(1-(5-(2,6-dioxopiperidin-3-yl)pyridin-2-yl)piperidin-4-yl)acetyl)piperazin-1-yl)-3-fluorophenyl)amino)pyrimidin-4-yl)thiazol-4-yl)-2-fluorophenyl)propane-1-sulfonamide, (S)—N-(3-(2-(tert-butyl)-5-(2-((4-(4-((1-(5-(2,6-dioxopiperidin-3-yl)pyridin-2-yl)piperidin-4-yl)methyl)piperazin-1-yl)phenyl)amino)pyrimidin-4-yl)thiazol-4-yl)-2-fluorophenyl)propane-1-sulfonamide, (S)—N-(3-(2-(tert-butyl)-5-(2-((4-(4-(2-(1-(4-((2,6-dioxopiperidin-3-yl)amino)phenyl)piperidin-4-yl)acetyl)piperazin-1-yl)-3-fluorophenyl)amino)pyrimidin-4-yl)thiazol-4-yl)-2-fluorophenyl)propane-1-sulfonamide, (S)—N-(3-(2-(tert-butyl)-5-(2-((4-(4-((1-(5-(2,6-dioxopiperidin-3-yl)pyridin-2-yl)piperidin-4-yl)methyl)piperazin-1-yl)-3-fluorophenyl)amino)pyrimidin-4-yl)thiazol-4-yl)-2-fluorophenyl)propane-1-sulfonamide, (S)—N-(3-(5-(2-((4-(1-(2-(1-(4-((2,6-dioxopiperidin-3-yl)amino)phenyl)piperidin-4-yl)acetyl)piperidin-4-yl)phenyl)amino)pyrimidin-4-yl)-2-methylthiazol-4-yl)-2-fluorophenyl)propane-1-sulfonamide, rac-(3S)-3-{6-[4-({4-[6-({4-[4-(3-{[ethyl(methyl)sulfamoyl]amino}-2-fluorophenyl)-2-methyl-1,3-thiazol-5-yl]pyrimidin-2-yl}amino)pyridin-3-yl]piperazin-1-yl}methyl)piperidin-1-yl]pyridin-3-yl}piperidine-2,6-dione, rac-(3S)-3-[6-(4-{4-[6-({4-[2-tert-butyl-4-(3-{[ethyl(methyl)sulfamoyl]amino}-2-fluorophenyl)-1,3-thiazol-5-yl]pyrimidin-2-yl}amino)pyridin-3-yl]piperazine-1-carbonyl}-4-methylpiperidin-1-yl)pyridin-3-yl]piperidine-2,6-dione, (3R)-3-{4-[4-({4-[6-({4-[4-(3-{[ethyl(methyl)sulfamoyl]amino}-2-fluorophenyl)-2-methyl-1,3-thiazol-5-yl]pyrimidin-2-yl}amino)pyridin-3-yl]piperazin-1-yl}methyl)piperidin-1-yl]phenyl}piperidine-2,6-dione, rac-(3S)-3-[6-(4-{4-[6-({4-[4-(3-{[ethyl(methyl)sulfamoyl]amino}-2-fluorophenyl)-2-methyl-1,3-thiazol-5-yl]pyrimidin-2-yl}amino)pyridin-3-yl]piperazine-1-carbonyl}piperidin-1-yl)pyridin-3-yl]piperidine-2,6-dione, rac-(3R)-3-{6-[4-(2-{4-[6-({4-[4-(3-{[ethyl(methyl)sulfamoyl]amino}-2-fluorophenyl)-2-methyl-1,3-thiazol-5-yl]pyrimidin-2-yl}amino)pyridin-3-yl]piperazin-1-yl}-2-oxoethyl)piperidin-1-yl]pyridin-3-yl}piperidine-2,6-dione, (S)—N-(3-(2-(tert-butyl)-5-(2-((4-(4-(1-(4-(2,6-dioxopiperidin-3-yl)phenyl)piperidine-4-carbonyl)piperazin-1-yl)phenyl)amino)pyrimidin-4-yl)thiazol-4-yl)-2-fluorophenyl)propane-1-sulfonamide, (S)—N-(3-(2-(tert-butyl)-5-(2-((4-(1-(1-(4-(2,6-dioxopiperidin-3-yl)phenyl)piperidine-4-carbonyl)piperidin-4-yl)phenyl)amino)pyrimidin-4-yl)thiazol-4-yl)-2-fluorophenyl)propane-1-sulfonamide, rac-(3S)-3-[4-(4-{4-[6-({4-[2-tert-butyl-4-(3-{[ethyl(methyl)sulfamoyl]amino}-2-fluorophenyl)-1,3-thiazol-5-yl]pyrimidin-2-yl}amino)pyridin-3-yl]piperazine-1-carbonyl}piperidin-1-yl)phenyl]piperidine-2,6-dione, (R)—N-(3-(2-(tert-butyl)-5-(2-((4-(1-((1-(5-(2,6-dioxopiperidin-3-yl)pyridin-2-yl)piperidin-4-yl)methyl)piperidin-4-yl)phenyl)amino)pyrimidin-4-yl)thiazol-4-yl)-2-fluorophenyl)propane-1-sulfonamide, rac-(3S)-3-{6-[4-({4-[6-({4-[4-(3-{[ethyl(methyl)sulfamoyl]amino}-2-fluorophenyl)-2-methyl-1,3-thiazol-5-yl]pyrimidin-2-yl}amino)pyridin-3-yl]piperazin-1-yl}methyl)-4-methylpiperidin-1-yl]pyridin-3-yl}piperidine-2,6-dione, rac-(3S)-3-{4-[4-({4-[6-({4-[4-(3-{[ethyl(methyl)sulfamoyl]amino}-2-fluorophenyl)-2-methyl-1,3-thiazol-5-yl]pyrimidin-2-yl}amino)pyridin-3-yl]piperazin-1-yl}methyl)-4-fluoropiperidin-1-yl]phenyl}piperidine-2,6-dione, (S)—N-(3-(5-(2-((4-(4-((1-(5-(2,6-dioxopiperidin-3-yl)pyridin-2-yl)-4-methylpiperidin-4-yl)methyl)piperazin-1-yl)phenyl)amino)pyrimidin-4-yl)-2-methylthiazol-4-yl)-2-fluorophenyl)propane-1-sulfonamide, (S)—N-(3-(5-(2-((4-(4-((1-(4-(2,6-dioxopiperidin-3-yl)phenyl)-4-fluoropiperidin-4-yl)methyl)piperazin-1-yl)phenyl)amino)pyrimidin-4-yl)-2-methylthiazol-4-yl)-2-fluorophenyl)propane-1-sulfonamide, rac-(3R)-3-{6-[6-({4-[6-({4-[2-tert-butyl-4-(3-{[ethyl(methyl)sulfamoyl]amino}-2-fluorophenyl)-1,3-thiazol-5-yl]pyrimidin-2-yl}amino)pyridin-3-yl]piperazin-1-yl}methyl)-2-azaspiro[3.3]heptan-2-yl]pyridin-3-yl}piperidine-2,6-dione, (3R)-3-{6-[4-({4-[6-({4-[2-tert-butyl-4-(3-{[ethyl(methyl)sulfamoyl]amino}-2-fluorophenyl)-1,3-thiazol-5-yl]pyrimidin-2-yl}amino)pyridin-3-yl]piperazin-1-yl}methyl)piperidin-1-yl]pyridin-3-yl}piperidine-2,6-dione, rac-(3R)-3-[2-(2-{4-[4-({4-[2-tert-butyl-4-(3-{[ethyl(methyl)sulfamoyl]amino}-2-fluorophenyl)-1,3-thiazol-5-yl]pyrimidin-2-yl}amino)-2-fluorophenyl]piperazin-1-yl}-2-oxoethyl)-1,2,3,4-tetrahydroisoquinolin-6-yl]piperidine-2,6-dione, and (3R)-3-{6-[4-({4-[6-({4-[4-(3-{[ethyl(methyl)sulfamoyl]amino}-2-fluorophenyl)-2-methyl-1,3-thiazol-5-yl]pyrimidin-2-yl}amino)pyridin-3-yl]piperazin-1-yl}methyl)piperidin-1-yl]pyridin-3-yl}piperidine-2,6-dione. In certain embodiments, the compound of Formula (I) is selected from the group consisting of (S)—N-(3-(1-(4-(4-((1-(4-(2,6-dioxopiperidin-3-yl)phenyl)piperidin-4-yl)methyl)piperazin-1-yl)phenyl)-3-(pyridin-4-yl)-1H-pyrazol-4-yl)-2-fluorophenyl)propane-1-sulfonamide, (R)—N-(3-(1-(4-(4-((1-(4-(2,6-dioxopiperidin-3-yl)phenyl)piperidin-4-yl)methyl)piperazin-1-yl)phenyl)-3-(pyridin-4-yl)-1H-pyrazol-4-yl)-2-fluorophenyl)propane-1-sulfonamide, and (S)—N-(3-(1-(4-(4-((1-(5-(2,6-dioxopiperidin-3-yl)pyridin-2-yl)piperidin-4-yl)methyl)piperazin-1-yl)phenyl)-3-(pyridin-4-yl)-1H-pyrazol-4-yl)-2-fluorophenyl)propane-1-sulfonamide. In certain embodiments, the compound of Formula (I) is selected from the group consisting of (R)—N-(3-(1-(4-(4-(1-(4-(2,6-dioxopiperidin-3-yl)phenyl)piperidine-4-carbonyl)piperazine-1-carbonyl)phenyl)-3-(pyridin-4-yl)-1H-pyrazol-4-yl)-2-fluorophenyl)propane-1-sulfonamide, (S)—N-(3-(1-(4-(4-((1-(4-(2,6-dioxopiperidin-3-yl)phenyl)piperidin-4-yl)methyl)piperazine-1-carbonyl)phenyl)-3-(pyridin-4-yl)-1H-pyrazol-4-yl)-2-fluorophenyl)propane-1-sulfonamide, (S)—N-(3-(1-(4-(4-(1-(4-(2,6-dioxopiperidin-3-yl)phenyl)piperidine-4-carbonyl)piperazin-1-yl)phenyl)-3-(pyridin-4-yl)-1H-pyrazol-4-yl)-2-fluorophenyl)propane-1-sulfonamide, (R)—N-(3-(1-(4-((1-(1-(5-(2,6-dioxopiperidin-3-yl)pyridin-2-yl)piperidine-4-carbonyl)piperidin-4-yl)oxy)phenyl)-3-(pyridin-4-yl)-1H-pyrazol-4-yl)-2-fluorophenyl)propane-1-sulfonamide, (S)—N-(3-(1-(4-(4-((1-(5-(2,6-dioxopiperidin-3-yl)pyridin-2-yl)-4-methylpiperidin-4-yl)methyl)piperazin-1-yl)phenyl)-3-(pyridin-4-yl)-1H-pyrazol-4-yl)-2-fluorophenyl)propane-1-sulfonamide, (R)—N-(3-(1-(4-(4-((1-(4-(2,6-dioxopiperidin-3-yl)phenyl)piperidin-4-yl)methyl)piperazine-1-carbonyl)phenyl)-3-(pyridin-4-yl)-1H-pyrazol-4-yl)-2-fluorophenyl)propane-1-sulfonamide, (S)—N-(3-(1-(1-(1-(1-(4-(2,6-dioxopiperidin-3-yl)phenyl)piperidine-4-carbonyl)piperidine-4-carbonyl)piperidin-4-yl)-3-(pyridin-4-yl)-1H-pyrazol-4-yl)-2-fluorophenyl)propane-1-sulfonamide, (S)—N-(3-(1-(4-(4-((1-(4-(2,6-dioxopiperidin-3-yl)-3-fluorophenyl)piperidin-4-yl)methyl)piperazin-1-yl)phenyl)-3-(pyridin-4-yl)-1H-pyrazol-4-yl)-2-fluorophenyl)propane-1-sulfonamide, (S)—N-(3-(1-(4-(1-((1-(4-(2,6-dioxopiperidin-3-yl)-2-fluorophenyl)piperidin-4-yl)methyl)piperidin-4-yl)phenyl)-3-(pyridin-4-yl)-1H-pyrazol-4-yl)-2-fluorophenyl)propane-1-sulfonamide, (S)—N-(3-(1-(1-(1-(1-(5-(2,6-dioxopiperidin-3-yl)pyridin-2-yl)piperidine-4-carbonyl)piperidine-4-carbonyl)piperidin-4-yl)-3-(pyridin-4-yl)-1H-pyrazol-4-yl)-2-fluorophenyl)propane-1-sulfonamide, (S)—N-(3-(1-(4-(4-((1-(4-(2,6-dioxopiperidin-3-yl)-2-fluorophenyl)piperidin-4-yl)methyl)piperazin-1-yl)phenyl)-3-(pyridin-4-yl)-1H-pyrazol-4-yl)-2-fluorophenyl)propane-1-sulfonamide, (S)—N-(3-(1-(6-(4-((1-(4-(2,6-dioxopiperidin-3-yl)phenyl)piperidin-4-yl)methyl)piperazin-1-yl)pyridin-3-yl)-3-(pyridin-4-yl)-1H-pyrazol-4-yl)-2-fluorophenyl)propane-1-sulfonamide, (R)—N-(3-(1-(4-(4-(2-(1-(5-(2,6-dioxopiperidin-3-yl)pyridin-2-yl)piperidin-4-yl)acetyl)piperazine-1-carbonyl)phenyl)-3-(pyridin-4-yl)-1H-pyrazol-4-yl)-2-fluorophenyl)propane-1-sulfonamide, (S)—N-(3-(1-(1-(1-((1-(5-(2,6-dioxopiperidin-3-yl)pyridin-2-yl)piperidin-4-yl)methyl)piperidine-4-carbonyl)piperidin-4-yl)-3-(pyridin-4-yl)-1H-pyrazol-4-yl)-2-fluorophenyl)propane-1-sulfonamide, (S)—N-(3-(1-(4-(4-((1-(5-(2,6-dioxopiperidin-3-yl)pyridin-2-yl)piperidin-4-yl)methyl)piperazin-1-yl)phenyl)-3-(pyridin-4-yl)-1H-pyrazol-4-yl)-2-fluorophenyl)propane-1-sulfonamide, (S)—N-(3-(1-(6-(4-((1-(5-(2,6-dioxopiperidin-3-yl)pyridin-2-yl)piperidin-4-yl)methyl)piperazin-1-yl)pyridin-3-yl)-3-(pyridin-4-yl)-1H-pyrazol-4-yl)-2-fluorophenyl)propane-1-sulfonamide, (S)—N-(3-(1-(4-(1-((1-(4-(2,6-dioxopiperidin-3-yl)phenyl)piperidin-4-yl)methyl)piperidin-4-yl)phenyl)-3-(pyridin-4-yl)-1H-pyrazol-4-yl)-2-fluorophenyl)propane-1-sulfonamide, (S)—N-(3-(1-(4-(1-((1-(4-(2,6-dioxopiperidin-3-yl)phenyl)piperidin-4-yl)methyl)piperidin-4-yl)phenyl)-3-(pyridin-4-yl)-1H-pyrazol-4-yl)-2-fluorophenyl)propane-1-sulfonamide, (S)—N-(3-(1-(4-(1-((1-(5-(2,6-dioxopiperidin-3-yl)pyridin-2-yl)piperidin-4-yl)methyl)piperidin-4-yl)phenyl)-3-(pyridin-4-yl)-1H-pyrazol-4-yl)-2-fluorophenyl)propane-1-sulfonamide, (S)—N-(3-(1-(4-(4-((1-(4-(2,6-dioxopiperidin-3-yl)phenyl)piperidin-4-yl)methyl)piperazin-1-yl)phenyl)-3-(pyridin-4-yl)-1H-pyrazol-4-yl)-2-fluorophenyl)propane-1-sulfonamide, (S)—N-(3-(1-(1-(1-(1-(5-(2,6-dioxopiperidin-3-yl)pyridin-2-yl)piperidine-4-carbonyl)-4-methylpiperidine-4-carbonyl)piperidin-4-yl)-3-(pyridin-4-yl)-1H-pyrazol-4-yl)-2-fluorophenyl)propane-1-sulfonamide, (S)—N-(3-(1-(4-(1-((1-(4-(2,6-dioxopiperidin-3-yl)-3-fluorophenyl)piperidin-4-yl)methyl)piperidin-4-yl)phenyl)-3-(pyridin-4-yl)-1H-pyrazol-4-yl)-2-fluorophenyl)propane-1-sulfonamide, and (R)—N-(3-(1-(4-(4-((1-(5-(2,6-dioxopiperidin-3-yl)pyridin-2-yl)piperidin-4-yl)methyl)piperazin-1-yl)phenyl)-3-(pyridin-4-yl)-1H-pyrazol-4-yl)-2-fluorophenyl)propane-1-sulfonamide.

In certain embodiments, the compound of Formula (II) is selected from the group consisting of (S)—N-(3-(5-(2-aminopyrimidin-4-yl)-2-(1-(1-(1-(5-(2,6-dioxopiperidin-3-yl)pyridin-2-yl)piperidine-4-carbonyl)piperidine-4-carbonyl)piperidin-4-yl)thiazol-4-yl)-2-fluorophenyl)propane-1-sulfonamide, (S)—N-(3-(5-(2-aminopyrimidin-4-yl)-2-(1-(1-(1-(5-(2,6-dioxopiperidin-3-yl)pyridin-2-yl)-4-methylpiperidine-4-carbonyl)piperidine-4-carbonyl)piperidin-4-yl)thiazol-4-yl)-2-fluorophenyl)propane-1-sulfonamide, (S)—N-(3-(5-(2-aminopyrimidin-4-yl)-2-(1-(1-(1-(5-(2,6-dioxopiperidin-3-yl)pyridin-2-yl)piperidine-4-carbonyl)-4-methylpiperidine-4-carbonyl)piperidin-4-yl)thiazol-4-yl)-2-fluorophenyl)propane-1-sulfonamide, (R)—N-(3-(5-(2-aminopyrimidin-4-yl)-2-(1-(1-(1-(4-(2,6-dioxopiperidin-3-yl)phenyl)piperidine-4-carbonyl)-4-methylpiperidine-4-carbonyl)piperidin-4-yl)thiazol-4-yl)-2-fluorophenyl)propane-1-sulfonamide, (S)—N-(3-(2-(1-(1-(1-(4-(2,6-dioxopiperidin-3-yl)phenyl)piperidine-4-carbonyl)piperidine-4-carbonyl)piperidin-4-yl)-5-(pyrimidin-4-yl)thiazol-4-yl)-2-fluorophenyl)propane-1-sulfonamide, (S)—N-(3-(2-(1-(1-(1-(5-(2,6-dioxopiperidin-3-yl)pyridin-2-yl)piperidine-4-carbonyl)-4-methylpiperidine-4-carbonyl)piperidin-4-yl)-5-(pyrimidin-4-yl)thiazol-4-yl)-2-fluorophenyl)propane-1-sulfonamide, and (S)—N-(3-(2-(1-(1-(1-(5-(2,6-dioxopiperidin-3-yl)pyridin-2-yl)piperidine-4-carbonyl)-4-methylpiperidine-4-carbonyl)piperidin-4-yl)-5-(pyridin-4-yl)thiazol-4-yl)-2-fluorophenyl)propane-1-sulfonamide. In certain embodiments, the compound of Formula (II) is selected from the group consisting of (R)—N-(3-(5-(2-aminopyrimidin-4-yl)-2-(3-(2-(4-(3-(2-(2,6-dioxopiperidin-3-yl)-1,3-dioxoisoindolin-4-yl)propyl)piperazin-1-yl)acetyl)-3-azaspiro[5.5]undecan-9-yl)thiazol-4-yl)-2-fluorophenyl)propane-1-sulfonamide, N-(3-(5-(2-aminopyrimidin-4-yl)-2-(1-((1S,4r)-4-((6-((S)-2,6-dioxopiperidin-3-yl)-3,4-dihydroisoquinolin-2(1H)-yl)methyl)cyclohexane-1-carbonyl)piperidin-4-yl)thiazol-4-yl)-2-fluorophenyl)propane-1-sulfonamide, (S)—N-(3-(2-(1-(1-((1-(5-(2,6-dioxopiperidin-3-yl)pyridin-2-yl)piperidin-4-yl)methyl)piperidine-4-carbonyl)piperidin-4-yl)-5-(pyrimidin-4-yl)thiazol-4-yl)-2-fluorophenyl)propane-1-sulfonamide, (R)—N-(3-(5-(2-aminopyrimidin-4-yl)-2-(1-(2-(4-(2-(2,6-dioxopiperidin-3-yl)-1,3-dioxoisoindolin-5-yl)piperazin-1-yl)acetyl)-4-methylpiperidin-4-yl)thiazol-4-yl)-2-fluorophenyl)propane-1-sulfonamide, (R)—N-(3-(5-(2-aminopyrimidin-4-yl)-2-(1-(2-(1′-(2-(2,6-dioxopiperidin-3-yl)-1,3-dioxoisoindolin-5-yl)-[1,4′-bipiperidin]-4-yl)acetyl)-4-methylpiperidin-4-yl)thiazol-4-yl)-2-fluorophenyl)propane-1-sulfonamide, (S)—N-(3-(5-(2-aminopyrimidin-4-yl)-2-(1-(1-((1-(5-(2,6-dioxopiperidin-3-yl)pyridin-2-yl)piperidin-4-yl)methyl)piperidine-4-carbonyl)-4-methylpiperidin-4-yl)thiazol-4-yl)-2-fluorophenyl)propane-1-sulfonamide, (S)—N-(3-(5-(2-aminopyrimidin-4-yl)-2-(1-((1-(2-(6-(2,6-dioxopiperidin-3-yl)-3,4-dihydroisoquinolin-2(1H)-yl)acetyl)piperidin-4-yl)methyl)piperidin-4-yl)thiazol-4-yl)-2-fluorophenyl)propane-1-sulfonamide, (S)—N-(3-(5-(2-aminopyrimidin-4-yl)-2-(3-((1-(4-(2,6-dioxopiperidin-3-yl)phenyl)piperidin-4-yl)methyl)-3-azaspiro[5.5]undecan-9-yl)thiazol-4-yl)-2-fluorophenyl)propane-1-sulfonamide, (S)—N-(3-(2-(1-(1-((1-(5-(2,6-dioxopiperidin-3-yl)pyridin-2-yl)piperidin-4-yl)methyl)piperidine-4-carbonyl)piperidin-4-yl)-5-(pyridin-4-yl)thiazol-4-yl)-2-fluorophenyl)propane-1-sulfonamide, (R)—N-(3-(5-(2-aminopyrimidin-4-yl)-2-(1-(2-(1′-(2-(2,6-dioxopiperidin-3-yl)-1,3-dioxoisoindolin-5-yl)-[1,4′-bipiperidin]-4-yl)acetyl)piperidin-4-yl)thiazol-4-yl)-2-fluorophenyl)propane-1-sulfonamide, (R)—N-(3-(5-(2-aminopyrimidin-4-yl)-2-(1-(2-(4-(2-(2,6-dioxopiperidin-3-yl)-1,3-dioxoisoindolin-5-yl)piperazin-1-yl)acetyl)piperidin-4-yl)thiazol-4-yl)-2-fluorophenyl)propane-1-sulfonamide, (R)—N-(3-(5-(2-aminopyrimidin-4-yl)-2-(2-(1-(1-(1-(2-(2,6-dioxopiperidin-3-yl)-1,3-dioxoisoindolin-5-yl)piperidin-4-yl)azetidine-3-carbonyl)piperidin-4-yl)propan-2-yl)thiazol-4-yl)-2-fluorophenyl)propane-1-sulfonamide, N-(3-(5-(2-aminopyrimidin-4-yl)-2-(1-((1-((1R,4r)-4-(3-((R)-2,6-dioxopiperidin-3-yl)phenoxy)cyclohexane-1-carbonyl)piperidin-4-yl)methyl)piperidin-4-yl)thiazol-4-yl)-2-fluorophenyl)propane-1-sulfonamide, (R)—N-(3-(5-(2-aminopyrimidin-4-yl)-2-(3-((1-(5-(2,6-dioxopiperidin-3-yl)pyridin-2-yl)piperidin-4-yl)methyl)-3-azaspiro[5.5]undecan-9-yl)thiazol-4-yl)-2-fluorophenyl)propane-1-sulfonamide, (S)—N-(3-(5-(2-aminopyrimidin-4-yl)-2-(1-(1-(2-(1-(4-((2,6-dioxopiperidin-3-yl)amino)-2-fluorophenyl)-4-hydroxypiperidin-4-yl)acetyl)azetidin-3-yl)piperidin-4-yl)thiazol-4-yl)-2-fluorophenyl)propane-1-sulfonamide, (S)—N-(3-(5-(2-aminopyrimidin-4-yl)-2-(4-(1-(2-(1-(5-(2,6-dioxopiperidin-3-yl)pyridin-2-yl)piperidin-4-yl)acetyl)piperidin-4-yl)phenyl)thiazol-4-yl)-2-fluorophenyl)propane-1-sulfonamide, (S)—N-(3-(5-(2-aminopyrimidin-4-yl)-2-(4-(4-((1-(4-(2,6-dioxopiperidin-3-yl)phenyl)piperidin-4-yl)methyl)piperazin-1-yl)phenyl)thiazol-4-yl)-2-fluorophenyl)propane-1-sulfonamide, (S)—N-(3-(5-(2-aminopyrimidin-4-yl)-2-(4-(1-(2-(1-(4-((2,6-dioxopiperidin-3-yl)amino)phenyl)piperidin-4-yl)acetyl)piperidin-4-yl)phenyl)thiazol-4-yl)-2-fluorophenyl)propane-1-sulfonamide, (S)—N-(3-(5-(2-aminopyrimidin-4-yl)-2-(4-(1-(1-(5-(2,6-dioxopiperidin-3-yl)pyridin-2-yl)piperidine-4-carbonyl)piperidin-4-yl)phenyl)thiazol-4-yl)-2-fluorophenyl)propane-1-sulfonamide, (S)—N-(3-(5-(2-aminopyrimidin-4-yl)-2-(4-(1-((1-(4-(2,6-dioxopiperidin-3-yl)phenyl)piperidin-4-yl)methyl)piperidin-4-yl)phenyl)thiazol-4-yl)-2-fluorophenyl)propane-1-sulfonamide, N-(3-(5-(2-aminopyrimidin-4-yl)-2-(3-(((1S,4r)-4-((5-((S)-2,6-dioxopiperidin-3-yl)pyridin-2-yl)oxy)cyclohexyl)methyl)-3-azaspiro[5.5]undecan-9-yl)thiazol-4-yl)-2-fluorophenyl)propane-1-sulfonamide, (R)—N-(3-(5-(2-aminopyrimidin-4-yl)-2-(4-(4-(1-(4-(2,6-dioxopiperidin-3-yl)phenyl)piperidine-4-carbonyl)piperazin-1-yl)phenyl)thiazol-4-yl)-2-fluorophenyl)propane-1-sulfonamide, (R)-5-(4-(9-(5-(2-aminopyrimidin-4-yl)-4-(2-fluoro-3-(propylsulfonamido)phenyl)thiazol-2-yl)-3-azaspiro[5.5]undecane-3-carbonyl)piperidin-1-yl)-N-(2,6-dioxopiperidin-3-yl)picolinamide, N-(3-(5-(2-aminopyrimidin-4-yl)-2-(3-(2-((S)-1-(5-((S)-2,6-dioxopiperidin-3-yl)pyridin-2-yl)pyrrolidin-3-yl)ethyl)-3-azaspiro[5.5]undecan-9-yl)thiazol-4-yl)-2-fluorophenyl)propane-1-sulfonamide, (S)—N-(3-(5-(2-aminopyrimidin-4-yl)-2-(1-(1-(2-(1-(4-(2,6-dioxopiperidin-3-yl)phenyl)piperidin-4-yl)acetyl)azetidin-3-yl)piperidin-4-yl)thiazol-4-yl)-2-fluorophenyl)propane-1-sulfonamide, (S)—N-(3-(5-(2-aminopyrimidin-4-yl)-2-(3-(1-(5-(2,6-dioxopiperidin-3-yl)pyridin-2-yl)piperidine-4-carbonyl)-3-azaspiro[5.5]undecan-9-yl)thiazol-4-yl)-2-fluorophenyl)propane-1-sulfonamide, (S)—N-(3-(5-(2-aminopyrimidin-4-yl)-2-(4-(1-((1-(5-(2,6-dioxopiperidin-3-yl)pyridin-2-yl)piperidin-4-yl)methyl)piperidin-4-yl)phenyl)thiazol-4-yl)-2-fluorophenyl)propane-1-sulfonamide, (S)—N-(3-(2-(1-(1-(1-(4-(2,6-dioxopiperidin-3-yl)phenyl)piperidine-4-carbonyl)piperidine-4-carbonyl)piperidin-4-yl)-5-(pyridin-4-yl)thiazol-4-yl)-2-fluorophenyl)propane-1-sulfonamide, (S)—N-(3-(2-(1-(1-((1-(5-(2,6-dioxopiperidin-3-yl)pyridin-2-yl)piperidin-4-yl)methyl)piperidine-4-carbonyl)piperidin-4-yl)-5-(2-(methylamino)pyrimidin-4-yl)thiazol-4-yl)-2-fluorophenyl)propane-1-sulfonamide, (R)—N-(3-(5-(2-aminopyrimidin-4-yl)-2-(2-(2-(1′-(2-(2,6-dioxopiperidin-3-yl)-1,3-dioxoisoindolin-5-yl)-[1,4′-bipiperidin]-4-yl)acetyl)-2-azaspiro[3.5]nonan-7-yl)thiazol-4-yl)-2-fluorophenyl)propane-1-sulfonamide, (S)—N-(3-(2-(1-(1-(1-(5-(2,6-dioxopiperidin-3-yl)pyridin-2-yl)piperidine-4-carbonyl)piperidine-4-carbonyl)piperidin-4-yl)-5-(pyridin-4-yl)thiazol-4-yl)-2-fluorophenyl)propane-1-sulfonamide, N-(3-(5-(2-aminopyrimidin-4-yl)-2-(1-((1-(((1S,4r)-4-(4-((S)-2,6-dioxopiperidin-3-yl)phenoxy)cyclohexyl)methyl)piperidin-4-yl)methyl)piperidin-4-yl)thiazol-4-yl)-2-fluorophenyl)propane-1-sulfonamide, (R)—N-(3-(5-(2-aminopyrimidin-4-yl)-2-(2-(1-(2-(2,6-dioxopiperidin-3-yl)-1,3-dioxoisoindolin-5-yl)piperidine-4-carbonyl)-2-azaspiro[3.5]nonan-7-yl)thiazol-4-yl)-2-fluorophenyl)propane-1-sulfonamide, (S)—N-(3-(2-(1-(1-(2-(1-(4-((2,6-dioxopiperidin-3-yl)amino)phenyl)piperidin-4-yl)acetyl)piperidine-4-carbonyl)piperidin-4-yl)-5-(pyrimidin-4-yl)thiazol-4-yl)-2-fluorophenyl)propane-1-sulfonamide, (S)—N-(3-(5-(2-aminopyrimidin-4-yl)-2-(2-(1-(1-(1-(5-(2,6-dioxopiperidin-3-yl)pyridin-2-yl)piperidine-4-carbonyl)piperidine-4-carbonyl)piperidin-4-yl)ethyl)thiazol-4-yl)-2-fluorophenyl)propane-1-sulfonamide, (R)—N-(3-(5-(2-aminopyrimidin-4-yl)-2-(2-(2-(4-(2-(2,6-dioxopiperidin-3-yl)-1,3-dioxoisoindolin-5-yl)piperazin-1-yl)acetyl)-2-azaspiro[3.5]nonan-7-yl)thiazol-4-yl)-2-fluorophenyl)propane-1-sulfonamide, (R)—N-(3-(5-(2-aminopyrimidin-4-yl)-2-(4-(4-(1-(5-(2,6-dioxopiperidin-3-yl)pyridin-2-yl)piperidine-4-carbonyl)piperazin-1-yl)phenyl)thiazol-4-yl)-2-fluorophenyl)propane-1-sulfonamide, (S)—N-(3-(2-(1-(1-(1-(5-(2,6-dioxopiperidin-3-yl)pyridin-2-yl)piperidine-4-carbonyl)piperidine-4-carbonyl)piperidin-4-yl)-5-(pyrimidin-4-yl)thiazol-4-yl)-2-fluorophenyl)propane-1-sulfonamide, (R)—N-(3-(5-(2-aminopyrimidin-4-yl)-2-(1-(1-(1-(4-(2,6-dioxopiperidin-3-yl)phenyl)piperidine-4-carbonyl)piperidine-4-carbonyl)piperidin-4-yl)thiazol-4-yl)-2-fluorophenyl)propane-1-sulfonamide, (R)—N-(3-(5-(2-aminopyrimidin-4-yl)-2-(4-(4-((1-(4-(2,6-dioxopiperidin-3-yl)phenyl)piperidin-4-yl)methyl)piperazin-1-yl)phenyl)thiazol-4-yl)-2-fluorophenyl)propane-1-sulfonamide, (S)—N-(3-(5-(2-aminopyrimidin-4-yl)-2-(1-((1-(1-(5-(2,6-dioxopiperidin-3-yl)pyridin-2-yl)piperidine-4-carbonyl)piperidin-4-yl)methyl)piperidin-4-yl)thiazol-4-yl)-2-fluorophenyl)propane-1-sulfonamide, (R)—N-(3-(5-(2-aminopyrimidin-4-yl)-2-(4-(4-(2-(1-(5-(2,6-dioxopiperidin-3-yl)pyridin-2-yl)piperidin-4-yl)acetyl)piperazin-1-yl)phenyl)thiazol-4-yl)-2-fluorophenyl)propane-1-sulfonamide, (R)—N-(3-(5-(2-aminopyrimidin-4-yl)-2-(3-(2-(1′-(2-(2,6-dioxopiperidin-3-yl)-1,3-dioxoisoindolin-5-yl)-[1,4′-bipiperidin]-4-yl)acetyl)-3-azaspiro[5.5]undecan-9-yl)thiazol-4-yl)-2-fluorophenyl)propane-1-sulfonamide, (S)—N-(3-(5-(2-aminopyrimidin-4-yl)-2-(1-(1-((1-(4-(2,6-dioxopiperidin-3-yl)phenyl)piperidin-4-yl)methyl)piperidine-4-carbonyl)-4-methylpiperidin-4-yl)thiazol-4-yl)-2-fluorophenyl)propane-1-sulfonamide, (S)—N-(3-(2-(1-(1-(1-(4-(2,6-dioxopiperidin-3-yl)phenyl)piperidine-4-carbonyl)-4-methylpiperidine-4-carbonyl)piperidin-4-yl)-5-(2-(isopropylamino)pyrimidin-4-yl)thiazol-4-yl)-2-fluorophenyl)propane-1-sulfonamide, (R)—N-(3-(5-(2-aminopyrimidin-4-yl)-2-(3-(2-(4-(2-(2,6-dioxopiperidin-3-yl)-1,3-dioxoisoindolin-5-yl)piperazin-1-yl)acetyl)-3-azaspiro[5.5]undecan-9-yl)thiazol-4-yl)-2-fluorophenyl)propane-1-sulfonamide, N-(3-(5-(2-aminopyrimidin-4-yl)-2-(1-((1-((1R,4r)-4-(4-((R)-2,6-dioxopiperidin-3-yl)phenoxy)cyclohexane-1-carbonyl)piperidin-4-yl)methyl)piperidin-4-yl)thiazol-4-yl)-2-fluorophenyl)propane-1-sulfonamide, (R)—N-(3-(5-(2-aminopyrimidin-4-yl)-2-(2-(6-(2-(2,6-dioxopiperidin-3-yl)-3-oxoisoindolin-5-yl)hexanoyl)-2-azaspiro[3.5]nonan-7-yl)thiazol-4-yl)-2-fluorophenyl)propane-1-sulfonamide, N-(3-(2-(1-(1-(1-(5-((S)-2,6-dioxopiperidin-3-yl)pyridin-2-yl)piperidine-4-carbonyl)piperidine-4-carbonyl)piperidin-4-yl)-5-(2-(((R)-1-hydroxypropan-2-yl)amino)pyrimidin-4-yl)thiazol-4-yl)-2-fluorophenyl)propane-1-sulfonamide, (S)—N-(3-(5-(2-aminopyrimidin-4-yl)-2-(4-(4-((1-(5-(2,6-dioxopiperidin-3-yl)pyridin-2-yl)piperidin-4-yl)methyl)piperazin-1-yl)phenyl)thiazol-4-yl)-2-fluorophenyl)propane-1-sulfonamide, rac-(3S)-3-{6-[4-(4-{4-[5-(2-aminopyrimidin-4-yl)-4-(3-{[ethyl(methyl)sulfamoyl]amino}-2-fluorophenyl)-1,3-thiazol-2-yl]piperidine-1-carbonyl}piperidine-1-carbonyl)piperidin-1-yl]pyridin-3-yl}piperidine-2,6-dione, rac-(3S)-3-{6-[4-(4-{4-[4-(3-{[ethyl(methyl)sulfamoyl]amino}-2-fluorophenyl)-5-{2-[(propan-2-yl)amino]pyrimidin-4-yl}-1,3-thiazol-2-yl]piperidine-1-carbonyl}piperidine-1-carbonyl)piperidin-1-yl]pyridin-3-yl}piperidine-2,6-dione, (S)—N-(3-(5-(2-aminopyrimidin-4-yl)-2-(1-(1-((1-(4-(2,6-dioxopiperidin-3-yl)phenyl)piperidin-4-yl)methyl)piperidine-4-carbonyl)piperidin-4-yl)thiazol-4-yl)-2-fluorophenyl)propane-1-sulfonamide, (S)—N-(3-(5-(2-aminopyrimidin-4-yl)-2-(1-(1-((1-(5-(2,6-dioxopiperidin-3-yl)pyridin-2-yl)piperidin-4-yl)methyl)piperidine-4-carbonyl)piperidin-4-yl)thiazol-4-yl)-2-fluorophenyl)propane-1-sulfonamide, (R)—N-(3-(5-(2-aminopyrimidin-4-yl)-2-(3-((1-(2-(2,6-dioxopiperidin-3-yl)-1-oxoisoindolin-5-yl)piperidin-4-yl)methyl)-3-azaspiro[5.5]undecan-9-yl)thiazol-4-yl)-2-fluorophenyl)propane-1-sulfonamide, (R)—N-(3-(5-(2-aminopyrimidin-4-yl)-2-(1-(2-(2-(2-(2,6-dioxopiperidin-3-yl)-1,3-dioxoisoindolin-5-yl)-2,7-diazaspiro[3.5]nonan-7-yl)acetyl)-4-methylpiperidin-4-yl)thiazol-4-yl)-2-fluorophenyl)propane-1-sulfonamide, (R)—N-(3-(5-(2-aminopyrimidin-4-yl)-2-(3-(1-(2-(2,6-dioxopiperidin-3-yl)-1,3-dioxoisoindolin-5-yl)piperidine-4-carbonyl)-3-azaspiro[5.5]undecan-9-yl)thiazol-4-yl)-2-fluorophenyl)propane-1-sulfonamide, (R)—N-(3-(2-(1-(2-(2-(2-(2,6-dioxopiperidin-3-yl)-1,3-dioxoisoindolin-5-yl)-2,7-diazaspiro[3.5]nonan-7-yl)acetyl)piperidin-4-yl)-5-(2-(methylamino)pyrimidin-4-yl)thiazol-4-yl)-2-fluorophenyl)propane-1-sulfonamide, (R)—N-(3-(5-(2-aminopyrimidin-4-yl)-2-(3-(6-(2-(2,6-dioxopiperidin-3-yl)-3-oxoisoindolin-5-yl)hexanoyl)-3-azaspiro[5.5]undecan-9-yl)thiazol-4-yl)-2-fluorophenyl)propane-1-sulfonamide, (R)—N-(3-(5-(2-aminopyrimidin-4-yl)-2-(1-(1-(2-(1-(5-(2,6-dioxopiperidin-3-yl)pyridin-2-yl)piperidin-4-yl)acetyl)-4-methylpiperidine-4-carbonyl)piperidin-4-yl)thiazol-4-yl)-2-fluorophenyl)propane-1-sulfonamide, (S)—N-(3-(2-(1-(1-(1-(5-(2,6-dioxopiperidin-3-yl)pyridin-2-yl)piperidine-4-carbonyl)piperidine-4-carbonyl)piperidin-4-yl)-5-(2-(methylamino)pyrimidin-4-yl)thiazol-4-yl)-2-fluorophenyl)propane-1-sulfonamide, (R)—N-(3-(5-(2-aminopyrimidin-4-yl)-2-(1-(1-(2-(1-(5-(2,6-dioxopiperidin-3-yl)pyridin-2-yl)piperidin-4-yl)acetyl)piperidine-4-carbonyl)piperidin-4-yl)thiazol-4-yl)-2-fluorophenyl)propane-1-sulfonamide, (S)—N-(3-(2-(1-(1-(1-(5-(2,6-dioxopiperidin-3-yl)pyridin-2-yl)piperidine-4-carbonyl)piperidine-4-carbonyl)piperidin-4-yl)-5-(2-(isopropylamino)pyrimidin-4-yl)thiazol-4-yl)-2-fluorophenyl)propane-1-sulfonamide, (S)—N-(3-(5-(2-(cyclopropylamino)pyrimidin-4-yl)-2-(1-(1-(1-(5-(2,6-dioxopiperidin-3-yl)pyridin-2-yl)piperidine-4-carbonyl)piperidine-4-carbonyl)piperidin-4-yl)thiazol-4-yl)-2-fluorophenyl)propane-1-sulfonamide, (S)—N-(3-(2-(1-(1-(1-(4-(2,6-dioxopiperidin-3-yl)phenyl)piperidine-4-carbonyl)-4-methylpiperidine-4-carbonyl)piperidin-4-yl)-5-(pyrimidin-4-yl)thiazol-4-yl)-2-fluorophenyl)propane-1-sulfonamide, N-(3-(5-(2-aminopyrimidin-4-yl)-2-(3-(2-((R)-1-(4-((S)-2,6-dioxopiperidin-3-yl)phenyl)pyrrolidin-3-yl)acetyl)-3-azaspiro[5.5]undecan-9-yl)thiazol-4-yl)-2-fluorophenyl)propane-1-sulfonamide, (R)—N-(3-(5-(2-aminopyrimidin-4-yl)-2-(2-(2-(2-(2-(2,6-dioxopiperidin-3-yl)-1,3-dioxoisoindolin-5-yl)-2,7-diazaspiro[3.5]nonan-7-yl)acetyl)-2-azaspiro[3.5]nonan-7-yl)thiazol-4-yl)-2-fluorophenyl)propane-1-sulfonamide, (R)—N-(3-(5-(2-aminopyrimidin-4-yl)-2-(1-((1-(2-(1-(5-(2,6-dioxopiperidin-3-yl)pyridin-2-yl)piperidin-4-yl)acetyl)piperidin-4-yl)methyl)piperidin-4-yl)thiazol-4-yl)-2-fluorophenyl)propane-1-sulfonamide, (R)—N-(3-(5-(2-aminopyrimidin-4-yl)-2-(1-(2-(2-(2-(2,6-dioxopiperidin-3-yl)-1,3-dioxoisoindolin-5-yl)-2,7-diazaspiro[3.5]nonan-7-yl)acetyl)piperidin-4-yl)thiazol-4-yl)-2-fluorophenyl)propane-1-sulfonamide, (R)—N-(3-(5-(2-aminopyrimidin-4-yl)-2-(3-(1-(1-(2,6-dioxopiperidin-3-yl)-3-methyl-2-oxo-2,3-dihydro-1H-benzo[d]imidazol-4-yl)piperidine-4-carbonyl)-3-azaspiro[5.5]undecan-9-yl)thiazol-4-yl)-2-fluorophenyl)propane-1-sulfonamide, (R)—N-(3-(2-(1-(1-((1-(4-(2,6-dioxopiperidin-3-yl)phenyl)piperidin-4-yl)methyl)piperidine-4-carbonyl)piperidin-4-yl)-5-(2-(methylamino)pyrimidin-4-yl)thiazol-4-yl)-2-fluorophenyl)propane-1-sulfonamide, (R)—N-(3-(5-(2-aminopyrimidin-4-yl)-2-(1-(1-(1-(2-(2,6-dioxopiperidin-3-yl)-1,3-dioxoisoindolin-5-yl)piperidin-4-yl)azetidine-3-carbonyl)piperidin-4-yl)thiazol-4-yl)-2-fluorophenyl)propane-1-sulfonamide, (R)—N-(3-(5-(2-aminopyrimidin-4-yl)-2-(2-(1-(1-(2-(2,6-dioxopiperidin-3-yl)-1,3-dioxoisoindolin-5-yl)piperidin-4-yl)azetidine-3-carbonyl)-2-azaspiro[3.5]nonan-7-yl)thiazol-4-yl)-2-fluorophenyl)propane-1-sulfonamide, (S)—N-(3-(5-(2-aminopyrimidin-4-yl)-2-(1-(1-((1-(1-(2,6-dioxopiperidin-3-yl)-3-methyl-2-oxo-2,3-dihydro-1H-benzo[d]imidazol-4-yl)piperidin-4-yl)methyl)piperidine-4-carbonyl)piperidin-4-yl)thiazol-4-yl)-2-fluorophenyl)propane-1-sulfonamide, (R)—N-(3-(5-(2-aminopyrimidin-4-yl)-2-(3-(2-(2-(2-(2,6-dioxopiperidin-3-yl)-1,3-dioxoisoindolin-5-yl)-2,7-diazaspiro[3.5]nonan-7-yl)acetyl)-3-azaspiro[5.5]undecan-9-yl)thiazol-4-yl)-2-fluorophenyl)propane-1-sulfonamide, (R)—N-(3-(5-(2-aminopyrimidin-4-yl)-2-(2-(1-(2-(2-(2-(2,6-dioxopiperidin-3-yl)-1,3-dioxoisoindolin-5-yl)-2,7-diazaspiro[3.5]nonan-7-yl)acetyl)piperidin-4-yl)propan-2-yl)thiazol-4-yl)-2-fluorophenyl)propane-1-sulfonamide, and (R)-4-((4-(5-(2-aminopyrimidin-4-yl)-4-(2-fluoro-3-(propylsulfonamido)phenyl)thiazol-2-yl)piperidin-1-yl)methyl)-N-(2,6-dioxopiperidin-3-yl)benzamide.

In certain embodiments of Formula (I), X¹ is carbon; X² is nitrogen, X³ is nitrogen; X⁴ is carbon; and X⁵ is hydrogen. In certain embodiments of Formula (I), R¹ is alkyl, dialkylamino, cycloalkyl, or heterocycloalkyl each unsubstituted or substituted with one or more halogen; R^{1a} is halogen; R^{1b} is hydrogen, haloalkyl or halogen; R² is —[Ar¹—Z¹]ₙ—Z²-Cy¹—Z³-L-UBM; Ar¹ is arylene or heteroarylene, each unsubstituted or substituted with one or more halogen; Z¹ is a bond; n is one; Z² is absent; Cy¹ is absent or heterocycloalkylene, unsubstituted or substituted with one or more alkyl or one or more halogen; Z³ is absent or C₁₋₁₀ alkylene; L is a linker according to -L¹-L²-L³-L⁴- or -L⁴-L³-L²-L¹-, wherein -L¹- is -Q¹- or -Q²-; each -L²-, -L³-, and -L⁴- is independently, absent, —C₁₋₈ alkylene-, or -Q¹-; each -Q¹- is a three- to seven-membered heterocycloalkylene comprising at least one nitrogen and is unsubstituted or substituted with one or more methyl or halogen; each -Q²- is a five- to thirteen-membered bicyclic heterocycloalkylene comprising at least one nitrogen, wherein the five- to thirteen-membered bicyclic heterocycloalkylene is optionally a spiro bicyclic heterocycloalkylene ring; and UBM is a ubiquitin ligase binding moiety; or a stereoisomer and/or pharmaceutical salt thereof.

In certain embodiments of Formula (I), R¹ is selected from the group consisting of propyl, ethylmethylamino, sec-butyl,

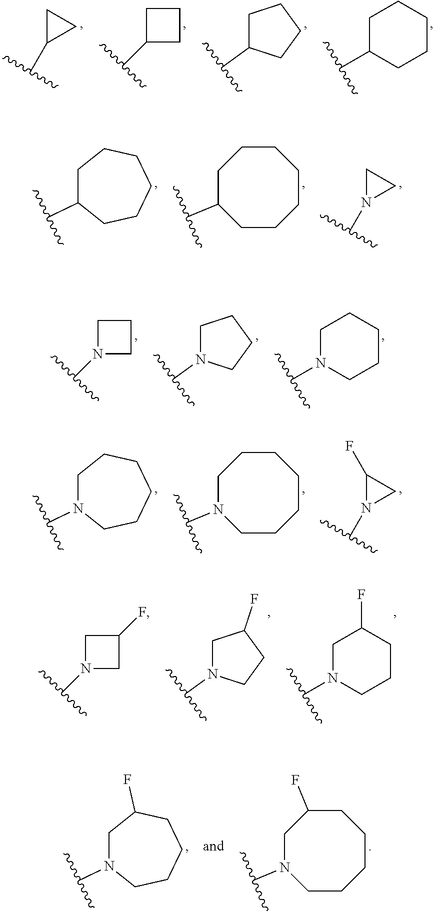

In one embodiment, R¹ is propyl. In one embodiment, R¹ is ethylmethylamino. In one embodiment, R¹ is sec-butyl. In one embodiment, R¹ is

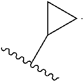 [SMILES: *C1CC1]

In one embodiment, R¹ is

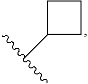 [SMILES: *C1CCC1]

In one embodiment, R¹ is

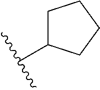 [SMILES: *C1CCCC1]

In one embodiment, R¹ is

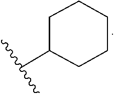 [SMILES: *C1CCCCC1]

In one embodiment, R¹ is

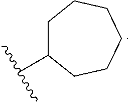 [SMILES: *C1CCCCCC1]

In one embodiment, R¹ is

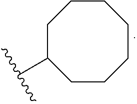 [SMILES: *C1CCCCCCC1]

In one embodiment, R¹ is

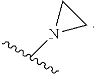 [SMILES: *N1CC1]

In one embodiment, R¹ is

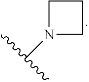 [SMILES: *N1CCC1]

In one embodiment, R¹ is

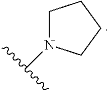 [SMILES: *N1CCCC1]

In one embodiment, R¹ is

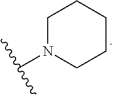 [SMILES: *N1CCCCC1]

In one embodiment, R¹ is

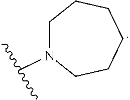 [SMILES: *N1CCCCCC1]

In one embodiment, R¹ is

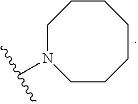 [SMILES: *N1CCCCCCC1]

In one embodiment, R¹ is

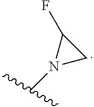 [SMILES: *N1CC1F]

In one embodiment, R¹ is

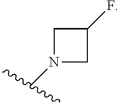 [SMILES: *N1CC(F)C1]

In one embodiment, R¹ is

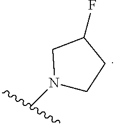 [SMILES: *N1CCC(F)C1]

In one embodiment, R¹ is

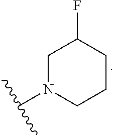 [SMILES: *N1CCCC(F)C1]

In one embodiment, R¹ is

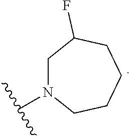 [SMILES: *N1CCCCC(F)C1]

In one embodiment, R¹ is

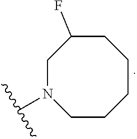 [SMILES: *N1CCCCCC(F)C1]

In certain embodiments of Formula (I), R^{1a} is chloro or fluoro; and R^{1b} is hydrogen, chloro, fluoro, or haloalkyl. In certain embodiments of Formula (I), Ar¹ is arylene or heteroarylene unsubstituted or substituted with one or two halogen. In certain embodiments of Formula (I), UBM binds an E3 ubiquitin ligase. In certain embodiments of Formula (I), UBM binds SCFβ-TRCP, VHL, MDM2, IAP, or CRBN. In certain embodiments of Formula (I), UBM binds CRBN. In certain embodiments of Formula (I), UBM binds CRBN and has the following chemical formula

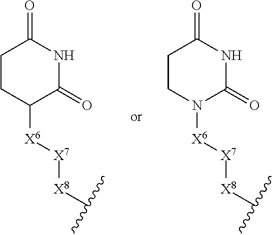

wherein X⁶ absent or NW; X⁷ absent or —C(O)—; and X⁸ is arylene or heteroarylene. In certain embodiments of Formula (I), UBM binds CRBN and is selected from the group consisting of

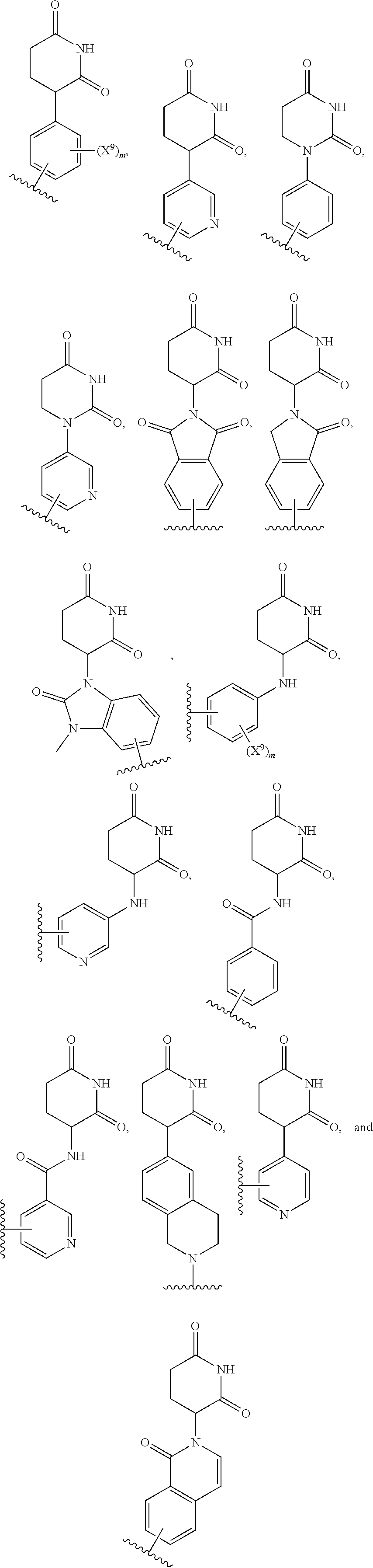

wherein X⁹ is absent or halogen; and m is one, two, three, or four. In one embodiment, UBM binds CRBN and is

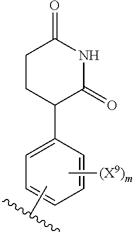

wherein X⁹ is absent or halogen; and m is one, two, three, or four. In one embodiment, UBM binds CRBN and is

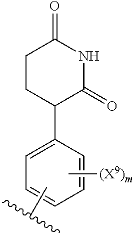

wherein X⁹ is absent. In one embodiment, UBM binds CRBN and is

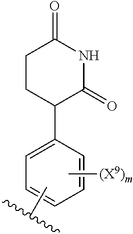

wherein X⁹ is halogen; and m is one. In one embodiment, UBM binds CRBN and is

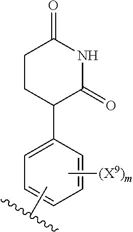

wherein X⁹ is halogen; and m is two.

In one embodiment, UBM binds CRBN and is

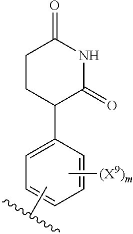

wherein X⁹ is halogen; and m is three. In one embodiment, UBM binds CRBN and is

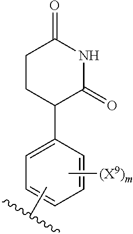

wherein X⁹ is halogen; and m is four. In one embodiment, UBM binds CRBN and is

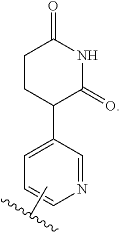

In one embodiment, UBM binds CRBN and is

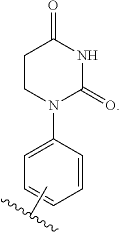

In one embodiment, UBM binds CRBN and is

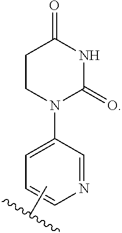

In one embodiment, UBM binds CRBN and is

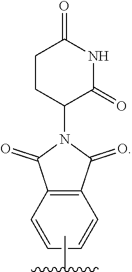

In one embodiment, UBM binds CRBN and is

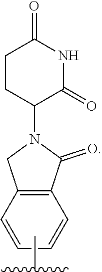

In one embodiment, UBM binds CRBN and is

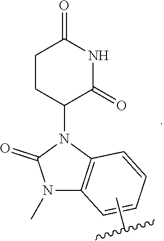

In one embodiment, UBM binds CRBN and is

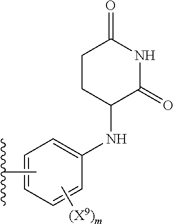

wherein X⁹ is absent or halogen; and m is one, two, three, or four. In one embodiment, UBM binds CRBN and is

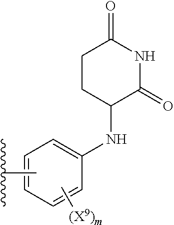

wherein X⁹ is absent. In one embodiment, UBM binds CRBN and is

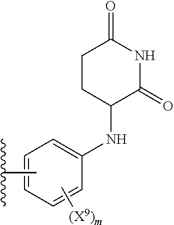

wherein X⁹ is halogen; and m is one. In one embodiment, UBM binds CRBN and is

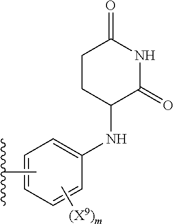

wherein X⁹ is halogen; and m is two. In one embodiment, UBM binds CRBN and is

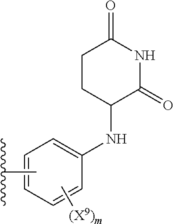

wherein X⁹ is halogen; and m is three. In one embodiment, UBM binds CRBN and is

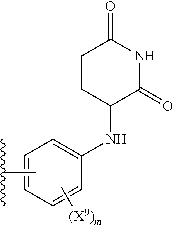

wherein X⁹ is halogen; and m is four. In one embodiment, UBM binds CRBN and is

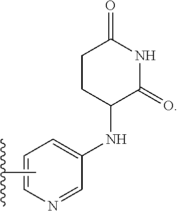

In one embodiment, UBM binds CRBN and is

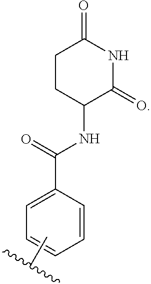

In one embodiment, UBM binds CRBN and is

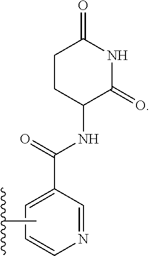

In one embodiment, UBM binds CRBN and is

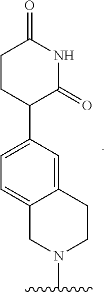 [SMILES: *N1CCc2cc(C3CCC(=O)NC3=O)ccc2C1]

In one embodiment, UBM binds CRBN and is

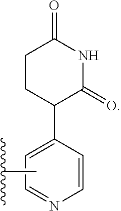

In one embodiment, UBM binds CRBN and is

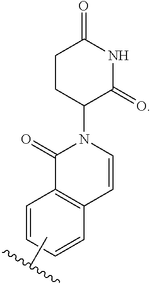

In certain embodiments of Formula (I), the compound is selected from the group consisting of (R)—N-(5-chloro-3-(1-(4-(4-((1-(5-(2,4-dioxotetrahydropyrimidin-1(2H)-yl)pyridin-2-yl)piperidin-4-yl)methyl)piperazin-1-yl)phenyl)-3-(pyridin-4-yl)-1H-pyrazol-4-yl)-2-fluorophenyl)-3-fluoropyrrolidine-1-sulfonamide; (R)—N-(5-chloro-3-(1-(4-(4-((1-(5-((R)-2,6-dioxopiperidin-3-yl)pyridin-2-yl)piperidin-4-yl)methyl)piperazin-1-yl)phenyl)-3-(pyridin-4-yl)-1H-pyrazol-4-yl)-2-fluorophenyl)-3-fluoropyrrolidine-1-sulfonamide; rac-(R)—N-(5-chloro-3-(1-(4-(4-((1-(5-(2,6-dioxopiperidin-3-yl)pyridin-2-yl)piperidin-4-yl)methyl)piperazin-1-yl)phenyl)-3-(pyridin-4-yl)-1H-pyrazol-4-yl)-2-fluorophenyl)pyrrolidine-1-sulfonamide; rac-(R)—N-(5-chloro-3-(1-(4-(4-((1-(5-(2,6-dioxopiperidin-3-yl)pyridin-2-yl)piperidin-4-yl)methyl)piperazin-1-yl)-2-fluorophenyl)-3-(pyridin-4-yl)-1H-pyrazol-4-yl)-2-fluorophenyl)pyrrolidine-1-sulfonamide; rac-(R)—N-(3-(1-(4-(4-((1-(5-(2,6-dioxopiperidin-3-yl)pyridin-2-yl)piperidin-4-yl)methyl)piperazin-1-yl)phenyl)-3-(pyridin-4-yl)-1H-pyrazol-4-yl)-2,5-difluorophenyl)pyrrolidine-1-sulfonamide; N-(5-chloro-3-(1-(4-((R)-4-((1-(5-((RS)-2,6-dioxopiperidin-3-yl)pyridin-2-yl)piperidin-4-yl)methyl)-3-methylpiperazin-1-yl)phenyl)-3-(pyridin-4-yl)-1H-pyrazol-4-yl)-2-fluorophenyl)pyrrolidine-1-sulfonamide; (R)—N-(3-(1-(4-(4-((1-(4-(2,6-dioxopiperidin-3-yl)-2-fluorophenyl)piperidin-4-yl)methyl)piperazin-1-yl)-2-fluorophenyl)-3-(pyridin-4-yl)-1H-pyrazol-4-yl)-2,5-difluorophenyl)cyclopentanesulfonamide;
rac-(R)—N-(3-(1-(4-(4-((1-(5-(2,6-dioxopiperidin-3-yl)pyridin-2-yl)piperidin-4-yl)methyl)piperazin-1-yl)-3-fluorophenyl)-3-(pyridin-4-yl)-1H-pyrazol-4-yl)-2,5-difluorophenyl)cyclopentanesulfonamide;
rac-(R)—N-(5-chloro-3-(1-(4-(4-((1-(5-(2,6-dioxopiperidin-3-yl)pyridin-2-yl)piperidin-4-yl)methyl)piperazin-1-yl)-3-fluorophenyl)-3-(pyridin-4-yl)-1H-pyrazol-4-yl)-2-fluorophenyl)cyclopentanesulfonamide;
(R)—N-(5-chloro-3-(1-(4-(4-((1-(4-(2,6-dioxopiperidin-3-yl)-2-fluorophenyl)piperidin-4-yl)methyl)piperazin-1-yl)-3-fluorophenyl)-3-(pyridin-4-yl)-1H-pyrazol-4-yl)-2-fluorophenyl)cyclopentanesulfonamide;
(R)—N-(5-chloro-3-(1-(4-(4-((1-(4-(2,6-dioxopiperidin-3-yl)-2-fluorophenyl)piperidin-4-yl)methyl)-1,4-diazepan-1-yl)-2-fluorophenyl)-3-(pyridin-4-yl)-1H-pyrazol-4-yl)-2-fluorophenyl)pyrrolidine-1-sulfonamide; (R)—N-(3-(1-(4-(4-((1-(4-(2,6-dioxopiperidin-3-yl)-2-fluorophenyl)piperidin-4-yl)methyl)-1,4-diazepan-1-yl)-2-fluorophenyl)-3-(pyridin-4-yl)-1H-pyrazol-4-yl)-2,5-difluorophenyl)pyrrolidine-1-sulfonamide; N-[5-chloro-3-(1-{4-[(3R)-4-[(1-{4-[(3R)-2,6-dioxopiperidin-3-yl]phenyl}piperidin-4-yl)methyl]-3-methylpiperazin-1-yl]phenyl}-3-(pyridin-4-yl)-1H-pyrazol-4-yl)-2-fluorophenyl]propane-1-sulfonamide; N-[5-chloro-3-(1-{4-[(3R)-4-[(1-{5-[(3RS)-2,6-dioxopiperidin-3-yl]pyridin-2-yl}piperidin-4-yl)methyl]-3-methylpiperazin-1-yl]phenyl}-3-(pyridin-4-yl)-1H-pyrazol-4-yl)-2-fluorophenyl]propane-1-sulfonamide; N-[5-chloro-3-(1-{4-[(3R)-4-({1-[4-(2,4-dioxo-1,3-diazinan-1-yl)phenyl]piperidin-4-yl}methyl)-3-methylpiperazin-1-yl]phenyl}-3-(pyridin-4-yl)-1H-pyrazol-4-yl)-2-fluorophenyl]propane-1-sulfonamide; N-[5-chloro-3-(1-{4-[(3S)-4-[(1-{4-[(3R)-2,6-dioxopiperidin-3-yl]phenyl}piperidin-4-yl)methyl]-3-methylpiperazin-1-yl]phenyl}-3-(pyridin-4-yl)-1H-pyrazol-4-yl)-2-fluorophenyl]propane-1-sulfonamide; N-[5-chloro-3-(1-{4-[(3S)-4-[(1-{5-[(3RS)-2,6-dioxopiperidin-3-yl]pyridin-2-yl}piperidin-4-yl)methyl]-3-methylpiperazin-1-yl]phenyl}-3-(pyridin-4-yl)-1H-pyrazol-4-yl)-2-fluorophenyl]propane-1-sulfonamide; N-[5-chloro-3-(1-{4-[(3S)-4-({1-[4-(2,4-dioxo-1,3-diazinan-1-yl)phenyl]piperidin-4-yl}methyl)-3-methylpiperazin-1-yl]phenyl}-3-(pyridin-4-yl)-1H-pyrazol-4-yl)-2-fluorophenyl]propane-1-sulfonamide; N-{5-chloro-3-[1-(4-{4-[(1-{4-[(3R)-2,6-dioxopiperidin-3-yl]phenyl}piperidin-4-yl)methyl]piperazin-1-yl}-2-fluorophenyl)-3-(pyridin-4-yl)-1H-pyrazol-4-yl]-2-fluorophenyl}pyrrolidine-1-sulfonamide; N-{5-chloro-3-[1-(4-{4-[(1-{4-[(3R)-2,6-dioxopiperidin-3-yl]phenyl}piperidin-4-yl)methyl]piperazin-1-yl}phenyl)-3-(pyridin-4-yl)-1H-pyrazol-4-yl]-2-fluorophenyl}pyrrolidine-1-sulfonamide; N-[5-chloro-3-(1-{4-[4-({1-[4-(2,4-dioxo-1,3-diazinan-1-yl)phenyl]piperidin-4-yl}methyl)piperazin-1-yl]phenyl}-3-(pyridin-4-yl)-1H-pyrazol-4-yl)-2-fluorophenyl]pyrrolidine-1-sulfonamide; rac-N-{5-chloro-3-[1-(4-{4-[(1-{4-[(3R)-2,6-dioxopiperidin-3-yl]phenyl}-4-fluoropiperidin-4-yl)methyl]piperazin-1-yl}phenyl)-3-(pyridin-4-yl)-1H-pyrazol-4-yl]-2-fluorophenyl}pyrrolidine-1-sulfonamide; N-{5-chloro-3-[1-(4-{4-[(1-{5-[(3R)-2,6-dioxopiperidin-3-yl]pyridin-2-yl}piperidin-4-yl)methyl]piperazin-1-yl}phenyl)-3-(pyridin-4-yl)-1H-pyrazol-4-yl]-2-fluorophenyl}pyrrolidine-1-sulfonamide; N-{5-chloro-3-[1-(5-{4-[(1-{4-[(3R)-2,6-dioxopiperidin-3-yl]phenyl}piperidin-4-yl)methyl]piperazin-1-yl}pyridin-2-yl)-3-(pyridin-4-yl)-1H-pyrazol-4-yl]-2-fluorophenyl}pyrrolidine-1-sulfonamide; N-[5-chloro-3-(1-{5-[4-({1-[4-(2,4-dioxo-1,3-diazinan-1-yl)phenyl]piperidin-4-yl}methyl)piperazin-1-yl]pyridin-2-yl}-3-(pyridin-4-yl)-1H-pyrazol-4-yl)-2-fluorophenyl]pyrrolidine-1-sulfonamide; rac-N-{5-chloro-3-[1-(5-{4-[(1-{5-[(3R)-2,6-dioxopiperidin-3-yl]pyridin-2-yl}piperidin-4-yl)methyl]piperazin-1-yl}pyridin-2-yl)-3-(pyridin-4-yl)-1H-pyrazol-4-yl]-2-fluorophenyl}pyrrolidine-1-sulfonamide; N-(3-(1-(4-((R)-4-((1-(5-((RS)-2,6-dioxopiperidin-3-yl)pyridin-2-yl)piperidin-4-yl)methyl)-3-methylpiperazin-1-yl)-2-fluorophenyl)-3-(pyridin-4-yl)-1H-pyrazol-4-yl)-2,5-difluorophenyl)pyrrolidine-1-sulfonamide; N-{5-chloro-3-[1-(5-{4-[(1-{5-[(3R)-2,6-dioxopiperidin-3-yl]pyridin-2-yl}piperidin-4-yl)methyl]piperazin-1-yl}pyridin-2-yl)-3-(pyridin-4-yl)-1H-pyrazol-4-yl]-2-fluorophenyl}pyrrolidine-1-sulfonamide; (3R)—N-{5-chloro-3-[1-(4-{4-[(1-{4-[(3R)-2,6-dioxopiperidin-3-yl]phenyl}piperidin-4-yl)methyl]piperazin-1-yl}phenyl)-3-(pyridin-4-yl)-1H-pyrazol-4-yl]-2-fluorophenyl}-3-fluoropyrrolidine-1-sulfonamide; (3R)—N-[5-chloro-3-(1-{4-[4-({1-[4-(2,4-dioxo-1,3-diazinan-1-yl)phenyl]piperidin-4-yl}methyl)piperazin-1-yl]phenyl}-3-(pyridin-4-yl)-1H-pyrazol-4-yl)-2-fluorophenyl]-3-fluoropyrrolidine-1-sulfonamide; (3R)—N-{5-chloro-3-[1-(4-{4-[(1-{5-[(3R)-2,6-dioxopiperidin-3-yl]pyridin-2-yl}piperidin-4-yl)methyl]piperazin-1-yl}phenyl)-3-(pyridin-4-yl)-1H-pyrazol-4-yl]-2-fluorophenyl}-3-fluoropyrrolidine-1-sulfonamide; (3R)—N-{5-chloro-3-[1-(5-{4-[(1-{4-[(3R)-2,6-dioxopiperidin-3-yl]phenyl}piperidin-4-yl)methyl]piperazin-1-yl}pyridin-2-yl)-3-(pyridin-4-yl)-1H-pyrazol-4-yl]-2-fluorophenyl}-3-fluoropyrrolidine-1-sulfonamide; (3R)—N-[5-chloro-3-(1-{5-[4-({1-[4-(2,4-dioxo-1,3-diazinan-1-yl)phenyl]piperidin-4-yl}methyl)piperazin-1-yl]pyridin-2-yl}-3-(pyridin-4-yl)-1H-pyrazol-4-yl)-2-fluorophenyl]-3-fluoropyrrolidine-1-sulfonamide; (3R)—N-{5-chloro-3-[1-(5-{4-[(1-{5-[(3RS)-2,6-dioxopiperidin-3-yl]pyridin-2-yl}piperidin-4-yl)methyl]piperazin-1-yl}pyridin-2-yl)-3-(pyridin-4-yl)-1H-pyrazol-4-yl]-2-fluorophenyl}-3-fluoropyrrolidine-1-sulfonamide; (3R)—N-{5-chloro-3-[1-(5-{4-[(1-{5-[(3R)-2,6-dioxopiperidin-3-yl]pyridin-2-yl}piperidin-4-yl)methyl]piperazin-1-yl}pyridin-2-yl)-3-(pyridin-4-yl)-1H-pyrazol-4-yl]-2-fluorophenyl}-3-fluoropyrrolidine-1-sulfonamide; (3R)—N-{5-chloro-3-[1-(5-{4-[(7-{4-[(3RS)-2,6-dioxopiperidin-3-yl]phenyl}-7-azaspiro[3.5]nonan-2-yl)methyl]piperazin-1-yl}pyridin-2-yl)-3-(pyridin-4-yl)-1H-pyrazol-4-yl]-2-fluorophenyl}-3-fluoropyrrolidine-1-sulfonamide; N-{3-[1-(4-{4-[(1-{4-[(3R)-2,6-dioxopiperidin-3-yl]phenyl}piperidin-4-yl)methyl]piperazin-1-yl}phenyl)-3-(pyridin-4-yl)-1H-pyrazol-4-yl]-2,5-difluorophenyl}pyrrolidine-1-sulfonamide; N-[3-(1-{4-[4-({1-[4-(2,4-dioxo-1,3-diazinan-1-yl)phenyl]piperidin-4-yl}methyl)piperazin-1-yl]phenyl}-3-(pyridin-4-yl)-1H-pyrazol-4-yl)-2,5-difluorophenyl]pyrrolidine-1-sulfonamide; N-{3-[1-(4-{4-[(1-{5-[(3R)-2,6-dioxopiperidin-3-yl]pyridin-2-yl}piperidin-4-yl)methyl]piperazin-1-yl}phenyl)-3-(pyridin-4-yl)-1H-pyrazol-4-yl]-2,5-difluorophenyl}pyrrolidine-1-sulfonamide; (3R)—N-{3-[1-(5-{4-[(1-{4-[(3R)-2,6-dioxopiperidin-3-yl]phenyl}piperidin-4-yl)methyl]piperazin-1-yl}pyridin-2-yl)-3-(pyridin-4-yl)-1H-pyrazol-4-yl]-2,5-difluorophenyl}-3-fluoropyrrolidine-1-sulfonamide; (3R)—N-[3-(1-{5-[4-({1-[4-(2,4-dioxo-1,3-diazinan-1-yl)phenyl]piperidin-4-yl}methyl)piperazin-1-yl]pyridin-2-yl}-3-(pyridin-4-yl)-1H-pyrazol-4-yl)-2,5-difluorophenyl]-3-fluoropyrrolidine-1-sulfonamide; (3R)—N-{3-[1-(5-{4-[(1-{5-[(3RS)-2,6-dioxopiperidin-3-yl]pyridin-2-yl}piperidin-4-yl)methyl]piperazin-1-yl}pyridin-2-yl)-3-(pyridin-4-yl)-1H-pyrazol-4-yl]-2,5-difluorophenyl}-3-fluoropyrrolidine-1-sulfonamide; (3R)—N-{3-[1-(5-{4-[(1-{5-[(3R)-2,6-dioxopiperidin-3-yl]pyridin-2-yl}piperidin-4-yl)methyl]piperazin-1-yl}pyridin-2-yl)-3-(pyridin-4-yl)-1H-pyrazol-4-yl]-2,5-difluorophenyl}-3-fluoropyrrolidine-1-sulfonamide; (3R)-3-(4-{4-[(4-{4-[4-(5-chloro-3-{[ethyl(methyl)sulfamoyl]amino}-2-fluorophenyl)-3-(pyridin-4-yl)-1H-pyrazol-1-yl]phenyl}piperazin-1-yl)methyl]piperidin-1-yl}phenyl)piperidine-2,6-dione; 1-(4-{4-[(4-{4-[4-(5-chloro-3-{[ethyl(methyl)sulfamoyl]amino}-2-fluorophenyl)-3-(pyridin-4-yl)-1H-pyrazol-1-yl]phenyl}piperazin-1-yl)methyl]piperidin-1-yl}phenyl)-1,3-diazinane-2,4-dione; rac-(3R)-3-(6-{4-[(4-{4-[4-(5-chloro-3-{[ethyl(methyl)sulfamoyl]amino}-2-fluorophenyl)-3-(pyridin-4-yl)-1H-pyrazol-1-yl]phenyl}piperazin-1-yl)methyl]piperidin-1-yl}pyridin-3-yl)piperidine-2,6-dione; (3R)-3-(6-{4-[(4-{4-[4-(5-chloro-3-{[ethyl(methyl)sulfamoyl]amino}-2-fluorophenyl)-3-(pyridin-4-yl)-1H-pyrazol-1-yl]phenyl}piperazin-1-yl)methyl]piperidin-1-yl}pyridin-3-yl)piperidine-2,6-dione; (3R)-3-(4-{4-[(4-{6-[4-(5-chloro-3-{[ethyl(methyl)sulfamoyl]amino}-2-fluorophenyl)-3-(pyridin-4-yl)-1H-pyrazol-1-yl]pyridin-3-yl}piperazin-1-yl)methyl]piperidin-1-yl}phenyl)piperidine-2,6-dione; 1-(4-{4-[(4-{6-[4-(5-chloro-3-{[ethyl(methyl)sulfamoyl]amino}-2-fluorophenyl)-3-(pyridin-4-yl)-1H-pyrazol-1-yl]pyridin-3-yl}piperazin-1-yl)methyl]piperidin-1-yl}phenyl)-1,3-diazinane-2,4-dione; rac-(3R)-3-(6-{4-[(4-{6-[4-(5-chloro-3-{[ethyl(methyl)sulfamoyl]amino}-2-fluorophenyl)-3-(pyridin-4-yl)-1H-pyrazol-1-yl]pyridin-3-yl}piperazin-1-yl)methyl]piperidin-1-yl}pyridin-3-yl)piperidine-2,6-dione; 1-(6-{4-[(4-{6-[4-(5-chloro-3-{[ethyl(methyl)sulfamoyl]amino}-2-fluorophenyl)-3-(pyridin-4-yl)-1H-pyrazol-1-yl]pyridin-3-yl}piperazin-1-yl)methyl]piperidin-1-yl}pyridin-3-yl)-1,3-diazinane-2,4-dione; (3R)-3-(6-{4-[(4-{6-[4-(5-chloro-3-{[ethyl(methyl)sulfamoyl]amino}-2-fluorophenyl)-3-(pyridin-4-yl)-1H-pyrazol-1-yl]pyridin-3-yl}piperazin-1-yl)methyl]piperidin-1-yl}pyridin-3-yl)piperidine-2,6-dione; (3R)—N-[5-chloro-3-(1-{4-[(3R)-4-({1-[4-(2,4-dioxo-1,3-diazinan-1-yl)phenyl]piperidin-4-yl}methyl)-3-methylpiperazin-1-yl]phenyl}-3-(pyridin-4-yl)-1H-pyrazol-4-yl)-2-fluorophenyl]-3-fluoropyrrolidine-1-sulfonamide; N-[5-chloro-3-(1-{4-[(3R)-4-[(1-{4-[(3R)-2,6-dioxopiperidin-3-yl]phenyl}piperidin-4-yl)methyl]-3-methylpiperazin-1-yl]phenyl}-3-(pyridin-4-yl)-1H-pyrazol-4-yl)-2-fluorophenyl]pyrrolidine-1-sulfonamide; (3R)—N-[5-chloro-3-(1-{4-[(3R)-4-[(1-{4-[(3R)-2,6-dioxopiperidin-3-yl]phenyl}piperidin-4-yl)methyl]-3-methylpiperazin-1-yl]phenyl}-3-(pyridin-4-yl)-1H-pyrazol-4-yl)-2-fluorophenyl]-3-fluoropyrrolidine-1-sulfonamide; (3R)—N-[5-chloro-3-(1-{4-[(3R)-4-[(1-{5-[(3RS)-2,6-dioxopiperidin-3-yl]pyridin-2-yl}piperidin-4-yl)methyl]-3-methylpiperazin-1-yl]phenyl}-3-(pyridin-4-yl)-1H-pyrazol-4-yl)-2-fluorophenyl]-3-fluoropyrrolidine-1-sulfonamide; N-[5-chloro-3-(1-{4-[(3R)-4-({1-[4-(2,4-dioxo-1,3-diazinan-1-yl)phenyl]piperidin-4-yl}methyl)-3-methylpiperazin-1-yl]phenyl}-3-(pyridin-4-yl)-1H-pyrazol-4-yl)-2-fluorophenyl]pyrrolidine-1-sulfonamide; (3R)—N-[5-chloro-3-(1-{5-[(3R)-4-[(1-{4-[(3R)-2,6-dioxopiperidin-3-yl]phenyl}piperidin-4-yl)methyl]-3-methylpiperazin-1-yl]pyridin-2-yl}-3-(pyridin-4-yl)-1H-pyrazol-4-yl)-2-fluorophenyl]-3-fluoropyrrolidine-1-sulfonamide; (3R)—N-[5-chloro-3-(1-{5-[(3R)-4-[(1-{5-[(3RS)-2,6-dioxopiperidin-3-yl]pyridin-2-yl}piperidin-4-yl)methyl]-3-methylpiperazin-1-yl]pyridin-2-yl}-3-(pyridin-4-yl)-1H-pyrazol-4-yl)-2-fluorophenyl]-3-fluoropyrrolidine-1-sulfonamide; (3R)—N-[5-chloro-3-(1-{5-[(3R)-4-({1-[4-(2,4-dioxo-1,3-diazinan-1-yl)phenyl]piperidin-4-yl}methyl)-3-methylpiperazin-1-yl]pyridin-2-yl}-3-(pyridin-4-yl)-1H-pyrazol-4-yl)-2-fluorophenyl]-3-fluoropyrrolidine-1-sulfonamide; N-[5-chloro-3-(1-{5-[(3R)-4-[(1-{5-[(3RS)-2,6-dioxopiperidin-3-yl]pyridin-2-yl}piperidin-4-yl)methyl]-3-methylpiperazin-1-yl]pyridin-2-yl}-3-(pyridin-4-yl)-1H-pyrazol-4-yl)-2-fluorophenyl]pyrrolidine-1-sulfonamide; N-[5-chloro-3-(1-{5-[(3R)-4-({1-[4-(2,4-dioxo-1,3-diazinan-1-yl)phenyl]piperidin-4-yl}methyl)-3-methylpiperazin-1-yl]pyridin-2-yl}-3-(pyridin-4-yl)-1H-pyrazol-4-yl)-2-fluorophenyl]pyrrolidine-1-sulfonamide; N-[5-chloro-3-(1-{5-[(3R)-4-[(1-{5-[(3R)-2,6-dioxopiperidin-3-yl]pyridin-2-yl}piperidin-4-yl)methyl]-3-methylpiperazin-1-yl]pyridin-2-yl}-3-(pyridin-4-yl)-1H-pyrazol-4-yl)-2-fluorophenyl]pyrrolidine-1-sulfonamide; N-[5-chloro-3-(1-{5-[(3R)-4-[(1-{4-[(3R)-2,6-dioxopiperidin-3-yl]phenyl}piperidin-4-yl)methyl]-3-methylpiperazin-1-yl]pyridin-2-yl}-3-(pyridin-4-yl)-1H-pyrazol-4-yl)-2-fluorophenyl]pyrrolidine-1-sulfonamide; (RS)—N-(5-chloro-3-(1-(4-(4-((1-(5-((R)-2,6-dioxopiperidin-3-yl)pyridin-2-yl)piperidin-4-yl)methyl)piperazin-1-yl)phenyl)-3-(pyridin-4-yl)-1H-pyrazol-4-yl)-2-fluorophenyl)butane-2-sulfonamide; rac-(2R)—N-[5-chloro-3-(1-{4-[4-({1-[4-(2,4-dioxo-1,3-diazinan-1-yl)phenyl]piperidin-4-yl}methyl)piperazin-1-yl]phenyl}-3-(pyridin-4-yl)-1H-pyrazol-4-yl)-2-fluorophenyl]butane-2-sulfonamide; rac-(R)—N-(5-chloro-3-(1-(4-(4-((1-(5-((S)-2,6-dioxopiperidin-3-yl)pyridin-2-yl)piperidin-4-yl)methyl)piperazin-1-yl)phenyl)-3-(pyridin-4-yl)-1H-pyrazol-4-yl)-2-fluorophenyl)butane-2-sulfonamide; (2RS)—N-{5-chloro-3-[1-(4-{4-[(1-{4-[(3R)-2,6-dioxopiperidin-3-yl]phenyl}piperidin-4-yl)methyl]piperazin-1-yl}phenyl)-3-(pyridin-4-yl)-1H-pyrazol-4-yl]-2-fluorophenyl}butane-2-sulfonamide; N-{5-chloro-3-[1-(4-{4-[(1-{5-[(3R)-2,6-dioxopiperidin-3-yl]pyridin-2-yl}piperidin-4-yl)methyl]piperazin-1-yl}phenyl)-3-(pyridin-4-yl)-1H-pyrazol-4-yl]-2-fluorophenyl}-3-fluoroazetidine-1-sulfonamide; rac-N-{5-chloro-3-[1-(4-{4-[(1-{5-[(3R)-2,6-dioxopiperidin-3-yl]pyridin-2-yl}piperidin-4-yl)methyl]piperazin-1-yl}phenyl)-3-(pyridin-4-yl)-1H-pyrazol-4-yl]-2-fluorophenyl}-3-fluoroazetidine-1-sulfonamide; N-{5-chloro-3-[1-(4-{4-[(1-{4-[(3R)-2,6-dioxopiperidin-3-yl]phenyl}piperidin-4-yl)methyl]piperazin-1-yl}phenyl)-3-(pyridin-4-yl)-1H-pyrazol-4-yl]-2-fluorophenyl}-3-fluoroazetidine-1-sulfonamide; (3R)—N-{5-chloro-3-[1-(4-{4-[(1-{5-[(3R)-2,6-dioxopiperidin-3-yl]pyridin-2-yl}piperidin-4-yl)methyl]piperazin-1-yl}-2-fluorophenyl)-3-(pyridin-4-yl)-1H-pyrazol-4-yl]-2-fluorophenyl}-3-fluoropyrrolidine-1-sulfonamide; (3R)—N-{5-chloro-3-[1-(4-{4-[(1-{5-[(3RS)-2,6-dioxopiperidin-3-yl]pyridin-2-yl}piperidin-4-yl)methyl]piperazin-1-yl}-2-fluorophenyl)-3-(pyridin-4-yl)-1H-pyrazol-4-yl]-2-fluorophenyl}-3-fluoropyrrolidine-1-sulfonamide; (3R)—N-{5-chloro-3-[1-(4-{4-[(1-{4-[(3R)-2,6-dioxopiperidin-3-yl]phenyl}piperidin-4-yl)methyl]piperazin-1-yl}-2-fluorophenyl)-3-(pyridin-4-yl)-1H-pyrazol-4-yl]-2-fluorophenyl}-3-fluoropyrrolidine-1-sulfonamide; rac-N-{5-chloro-3-[1-(4-{4-[(1-{5-[(3R)-2,6-dioxopiperidin-3-yl]pyridin-2-yl}piperidin-4-yl)methyl]piperazin-1-yl}-2-fluorophenyl)-3-(pyridin-4-yl)-1H-pyrazol-4-yl]-2-fluorophenyl}-3-fluoroazetidine-1-sulfonamide; N-{5-chloro-3-[1-(4-{4-[(1-{4-[(3R)-2,6-dioxopiperidin-3-yl]phenyl}piperidin-4-yl)methyl]piperazin-1-yl}-2-fluorophenyl)-3-(pyridin-4-yl)-1H-pyrazol-4-yl]-2-fluorophenyl}-3-fluoroazetidine-1-sulfonamide; N-[5-chloro-3-(1-{4-[4-({1-[4-(2,4-dioxo-1,3-diazinan-1-yl)phenyl]piperidin-4-yl}methyl)piperazin-1-yl]-2-fluorophenyl}-3-(pyridin-4-yl)-1H-pyrazol-4-yl)-2-fluorophenyl]-3-fluoroazetidine-1-sulfonamide; N-{3-[1-(4-{4-[(1-{4-[(3R)-2,6-dioxopiperidin-3-yl]phenyl}piperidin-4-yl)methyl]piperazin-1-yl}-2-fluorophenyl)-3-(pyridin-4-yl)-1H-pyrazol-4-yl]-2,5-difluorophenyl}pyrrolidine-1-sulfonamide; rac-N-{3-[1-(4-{4-[(1-{5-[(3R)-2,6-dioxopiperidin-3-yl]pyridin-2-yl}piperidin-4-yl)methyl]piperazin-1-yl}-2-fluorophenyl)-3-(pyridin-4-yl)-1H-pyrazol-4-yl]-2,5-difluorophenyl}pyrrolidine-1-sulfonamide; N-{3-[1-(5-{4-[(1-{4-[(3R)-2,6-dioxopiperidin-3-yl]phenyl}piperidin-4-yl)methyl]piperazin-1-yl}pyrimidin-2-yl)-3-(pyridin-4-yl)-1H-pyrazol-4-yl]-2,5-difluorophenyl}pyrrolidine-1-sulfonamide; rac-N-{3-[1-(5-{4-[(1-{5-[(3R)-2,6-dioxopiperidin-3-yl]pyridin-2-yl}piperidin-4-yl)methyl]piperazin-1-yl}pyrimidin-2-yl)-3-(pyridin-4-yl)-1H-pyrazol-4-yl]-2,5-difluorophenyl}pyrrolidine-1-sulfonamide; N-{5-chloro-3-[1-(4-{4-[(1-{4-[(3R)-2,6-dioxopiperidin-3-yl]-3-fluorophenyl}piperidin-4-yl)methyl]piperazin-1-yl}phenyl)-3-(pyridin-4-yl)-1H-pyrazol-4-yl]-2-fluorophenyl}pyrrolidine-1-sulfonamide; rac-N-{5-chloro-3-[1-(4-{4-[(1-{5-[(3R)-2,6-dioxopiperidin-3-yl]pyridin-2-yl}piperidin-4-yl)methyl]-1,4-diazepan-1-yl}-2-fluorophenyl)-3-(pyridin-4-yl)-1H-pyrazol-4-yl]-2-fluorophenyl}pyrrolidine-1-sulfonamide; N-{5-chloro-3-[1-(4-{4-[(1-{4-[(3R)-2,6-dioxopiperidin-3-yl]phenyl}piperidin-4-yl)methyl]-1,4-diazepan-1-yl}-2-fluorophenyl)-3-(pyridin-4-yl)-1H-pyrazol-4-yl]-2-fluorophenyl}pyrrolidine-1-sulfonamide; rac-N-{5-chloro-3-[1-(4-{4-[(1-{4-[(3R)-2,6-dioxopiperidin-3-yl]-2-fluorophenyl}piperidin-4-yl)methyl]piperazin-1-yl}phenyl)-3-(pyridin-4-yl)-1H-pyrazol-4-yl]-2-fluorophenyl}pyrrolidine-1-sulfonamide; rac-N-{5-chloro-3-[1-(4-{4-[(1-{4-[(3R)-2,6-dioxopiperidin-3-yl]-3-fluorophenyl}piperidin-4-yl)methyl]piperazin-1-yl}phenyl)-3-(pyridin-4-yl)-1H-pyrazol-4-yl]-2-fluorophenyl}pyrrolidine-1-sulfonamide; N-[5-(difluoromethyl)-3-[1-(4-{4-[(1-{4-[(3R)-2,6-dioxopiperidin-3-yl]phenyl}piperidin-4-yl)methyl]piperazin-1-yl}phenyl)-3-(pyridin-4-yl)-1H-pyrazol-4-yl]-2-fluorophenyl]pyrrolidine-1-sulfonamide; rac-N-[5-(difluoromethyl)-3-[1-(4-{4-[(1-{5-[(3R)-2,6-dioxopiperidin-3-yl]pyridin-2-yl}piperidin-4-yl)methyl]piperazin-1-yl}phenyl)-3-(pyridin-4-yl)-1H-pyrazol-4-yl]-2-fluorophenyl]pyrrolidine-1-sulfonamide; N-{5-chloro-3-[1-(6-{4-[(1-{4-[(3R)-2,6-dioxopiperidin-3-yl]phenyl}piperidin-4-yl)methyl]piperazin-1-yl}pyridin-3-yl)-3-(pyridin-4-yl)-1H-pyrazol-4-yl]-2-fluorophenyl}pyrrolidine-1-sulfonamide; rac-N-{5-chloro-3-[1-(6-{4-[(1-{5-[(3R)-2,6-dioxopiperidin-3-yl]pyridin-2-yl}piperidin-4-yl)methyl]piperazin-1-yl}pyridin-3-yl)-3-(pyridin-4-yl)-1H-pyrazol-4-yl]-2-fluorophenyl}pyrrolidine-1-sulfonamide; N-{5-chloro-3-[1-(4-{4-[(1-{4-[(3R)-2,6-dioxopiperidin-3-yl]phenyl}piperidin-4-yl)methyl]piperazin-1-yl}-2,5-difluorophenyl)-3-(pyridin-4-yl)-1H-pyrazol-4-yl]-2-fluorophenyl}pyrrolidine-1-sulfonamide; rac-N-{5-chloro-3-[1-(4-{4-[(1-{5-[(3R)-2,6-dioxopiperidin-3-yl]pyridin-2-yl}piperidin-4-yl)methyl]piperazin-1-yl}-2,5-difluorophenyl)-3-(pyridin-4-yl)-1H-pyrazol-4-yl]-2-fluorophenyl}pyrrolidine-1-sulfonamide; N-[5-chloro-3-(1-{4-[4-({1-[5-(2,4-dioxo-1,3-diazinan-1-yl)pyridin-2-yl]piperidin-4-yl}methyl)piperazin-1-yl]phenyl}-3-(pyridin-4-yl)-1H-pyrazol-4-yl)-2-fluorophenyl]pyrrolidine-1-sulfonamide; N-[5-(difluoromethyl)-3-(1-{4-[4-({1-[4-(2,4-dioxo-1,3-diazinan-1-yl)phenyl]piperidin-4-yl}methyl)piperazin-1-yl]-2-fluorophenyl}-3-(pyridin-4-yl)-1H-pyrazol-4-yl)-2-fluorophenyl]pyrrolidine-1-sulfonamide; rac-N-[5-(difluoromethyl)-3-[1-(4-{4-[(1-{5-[(3R)-2,6-dioxopiperidin-3-yl]pyridin-2-yl}piperidin-4-yl)methyl]piperazin-1-yl}-2-fluorophenyl)-3-(pyridin-4-yl)-1H-pyrazol-4-yl]-2-fluorophenyl]pyrrolidine-1-sulfonamide; N-[5-chloro-3-(1-{5-[4-({1-[4-(2,4-dioxo-1,3-diazinan-1-yl)phenyl]piperidin-4-yl}methyl)piperazin-1-yl]pyrimidin-2-yl}-3-(pyridin-4-yl)-1H-pyrazol-4-yl)-2-fluorophenyl]pyrrolidine-1-sulfonamide; rac-N-{5-chloro-3-[1-(5-{4-[(1-{5-[(3R)-2,6-dioxopiperidin-3-yl]pyridin-2-yl}piperidin-4-yl)methyl]piperazin-1-yl}pyrimidin-2-yl)-3-(pyridin-4-yl)-1H-pyrazol-4-yl]-2-fluorophenyl}pyrrolidine-1-sulfonamide; N-{5-chloro-3-[1-(5-{4-[(1-{4-[(3R)-2,6-dioxopiperidin-3-yl]phenyl}piperidin-4-yl)methyl]piperazin-1-yl}pyrimidin-2-yl)-3-(pyridin-4-yl)-1H-pyrazol-4-yl]-2-fluorophenyl}pyrrolidine-1-sulfonamide; N-[5-chloro-3-(1-{4-[4-({1-[4-(2,4-dioxo-1,3-diazinan-1-yl)phenyl]piperidin-4-yl}methyl)piperazin-1-yl]-3-fluorophenyl}-3-(pyridin-4-yl)-1H-pyrazol-4-yl)-2-fluorophenyl]pyrrolidine-1-sulfonamide; N-[5-(difluoromethyl)-3-[1-(4-{4-[(1-{4-[(3R)-2,6-dioxopiperidin-3-yl]phenyl}piperidin-4-yl)methyl]piperazin-1-yl}-2-fluorophenyl)-3-(pyridin-4-yl)-1H-pyrazol-4-yl]-2-fluorophenyl]pyrrolidine-1-sulfonamide; rac-N-{5-chloro-3-[1-(4-{4-[(1-{5-[(3R)-2,6-dioxopiperidin-3-yl]pyridin-2-yl}piperidin-4-yl)methyl]piperazin-1-yl}-3-fluorophenyl)-3-(pyridin-4-yl)-1H-pyrazol-4-yl]-2-fluorophenyl}pyrrolidine-1-sulfonamide; N-{5-chloro-3-[1-(4-{4-[(1-{4-[(3R)-2,6-dioxopiperidin-3-yl]phenyl}piperidin-4-yl)methyl]piperazin-1-yl}-3-fluorophenyl)-3-(pyridin-4-yl)-1H-pyrazol-4-yl]-2-fluorophenyl}pyrrolidine-1-sulfonamide; (3R)—N-[5-chloro-3-(1-{4-[4-({1-[4-(2,4-dioxo-1,3-diazinan-1-yl)phenyl]piperidin-4-yl}methyl)piperazin-1-yl]-3-fluorophenyl}-3-(pyridin-4-yl)-1H-pyrazol-4-yl)-2-fluorophenyl]-3-fluoropyrrolidine-1-sulfonamide; (3R)—N-{5-chloro-3-[1-(4-{4-[(1-{5-[(3RS)-2,6-dioxopiperidin-3-yl]pyridin-2-yl}piperidin-4-yl)methyl]piperazin-1-yl}-3-fluorophenyl)-3-(pyridin-4-yl)-1H-pyrazol-4-yl]-2-fluorophenyl}-3-fluoropyrrolidine-1-sulfonamide; (3R)—N-{5-chloro-3-[1-(4-{4-[(1-{4-[(3R)-2,6-dioxopiperidin-3-yl]phenyl}piperidin-4-yl)methyl]piperazin-1-yl}-3-fluorophenyl)-3-(pyridin-4-yl)-1H-pyrazol-4-yl]-2-fluorophenyl}-3-fluoropyrrolidine-1-sulfonamide; N-[5-chloro-3-(1-{4-[4-({1-[5-(2,4-dioxo-1,3-diazinan-1-yl)pyridin-2-yl]piperidin-4-yl}methyl)piperazin-1-yl]phenyl}-3-(pyridin-4-yl)-1H-pyrazol-4-yl)-2-fluorophenyl]-3-fluoroazetidine-1-sulfonamide; N-[5-chloro-3-(1-{4-[4-({1-[5-(2,4-dioxo-1,3-diazinan-1-yl)pyridin-2-yl]piperidin-4-yl}methyl)piperazin-1-yl]-2-fluorophenyl}-3-(pyridin-4-yl)-1H-pyrazol-4-yl)-2-fluorophenyl]-3-fluoroazetidine-1-sulfonamide; N-[5-chloro-3-(1-{4-[4-({1-[5-(2,4-dioxo-1,3-diazinan-1-yl)pyridin-2-yl]piperidin-4-yl}methyl)piperazin-1-yl]-2-fluorophenyl}-3-(pyridin-4-yl)-1H-pyrazol-4-yl)-2-fluorophenyl]pyrrolidine-1-sulfonamide; (3R)—N-[5-chloro-3-(1-{4-[4-({1-[5-(2,4-dioxo-1,3-diazinan-1-yl)pyridin-2-yl]piperidin-4-yl}methyl)piperazin-1-yl]-2-fluorophenyl}-3-(pyridin-4-yl)-1H-pyrazol-4-yl)-2-fluorophenyl]-3-fluoropyrrolidine-1-sulfonamide; N-{5-chloro-3-[1-(4-{4-[(1-{5-[(3R)-2,6-dioxopiperidin-3-yl]pyridin-2-yl}piperidin-4-yl)methyl]piperazin-1-yl}phenyl)-3-(pyridin-4-yl)-1H-pyrazol-4-yl]-2-fluorophenyl}cyclopentanesulfonamide; rac-N-{5-chloro-3-[1-(4-{4-[(1-{5-[(3R)-2,6-dioxopiperidin-3-yl]pyridin-2-yl}piperidin-4-yl)methyl]piperazin-1-yl}phenyl)-3-(pyridin-4-yl)-1H-pyrazol-4-yl]-2-fluorophenyl}cyclopentanesulfonamide; N-{5-chloro-3-[1-(4-{4-[(1-{4-[(3R)-2,6-dioxopiperidin-3-yl]phenyl}piperidin-4-yl)methyl]piperazin-1-yl}phenyl)-3-(pyridin-4-yl)-1H-pyrazol-4-yl]-2-fluorophenyl}cyclopentanesulfonamide; (3R)-3-(6-{4-[(4-{4-[4-(3-{[ethyl(methyl)sulfamoyl]amino}-2,5-difluorophenyl)-3-(pyridin-4-yl)-1H-pyrazol-1-yl]phenyl}piperazin-1-yl)methyl]piperidin-1-yl}pyridin-3-yl)piperidine-2,6-dione; rac-(3R)-3-(6-{4-[(4-{4-[4-(3-{[ethyl(methyl)sulfamoyl]amino}-2,5-difluorophenyl)-3-(pyridin-4-yl)-1H-pyrazol-1-yl]phenyl}piperazin-1-yl)methyl]piperidin-1-yl}pyridin-3-yl)piperidine-2,6-dione; (3R)-3-(4-{4-[(4-{4-[4-(3-{[ethyl(methyl)sulfamoyl]amino}-2,5-difluorophenyl)-3-(pyridin-4-yl)-1H-pyrazol-1-yl]phenyl}piperazin-1-yl)methyl]piperidin-1-yl}phenyl)piperidine-2,6-dione; N-[3-(1-{4-[(3R)-4-[(1-{4-[(3R)-2,6-dioxopiperidin-3-yl]phenyl}piperidin-4-yl)methyl]-3-methylpiperazin-1-yl]phenyl}-3-(pyridin-4-yl)-1H-pyrazol-4-yl)-2-fluorophenyl]propane-1-sulfonamide; (3R)-3-(6-{4-[(4-{4-[4-(3-{[ethyl(methyl)sulfamoyl]amino}-2-fluorophenyl)-3-(pyridin-4-yl)-1H-pyrazol-1-yl]-3-fluorophenyl}piperazin-1-yl)methyl]piperidin-1-yl}pyridin-3-yl)piperidine-2,6-dione; (3S)-3-(6-{4-[(4-{4-[4-(3-{[ethyl(methyl)sulfamoyl]amino}-2-fluorophenyl)-3-(pyridin-4-yl)-1H-pyrazol-1-yl]-2-fluorophenyl}piperazin-1-yl)methyl]piperidin-1-yl}pyridin-3-yl)piperidine-2,6-dione; rac-(R)—N-(3-(1-(4-(4-((4-(4-(2,6-dioxopiperidin-3-yl)phenyl)piperazin-1-yl)methyl)piperidin-1-yl)phenyl)-3-(pyridin-4-yl)-1H-pyrazol-4-yl)-2-fluorophenyl)propane-1-sulfonamide; N-(5-chloro-3-(1-(4-((3R,5R)-4-((1-(5-((RS)-2,6-dioxopiperidin-3-yl)pyridin-2-yl)piperidin-4-yl)methyl)-3,5-dimethylpiperazin-1-yl)-2-fluorophenyl)-3-(pyridin-4-yl)-1H-pyrazol-4-yl)-2-fluorophenyl)pyrrolidine-1-sulfonamide; N-{5-chloro-3-[1-(5-{4-[(1-{5-[(3S)-2,6-dioxopiperidin-3-yl]pyridin-2-yl}piperidin-4-yl)methyl]piperazin-1-yl}pyridin-2-yl)-3-(pyridin-4-yl)-1H-pyrazol-4-yl]-2-fluorophenyl}pyrrolidine-1-sulfonamide; N-{5-chloro-3-[1-(4-{4-[(1-{4-[(3S)-2,6-dioxopiperidin-3-yl]-3-fluorophenyl}piperidin-4-yl)methyl]piperazin-1-yl}phenyl)-3-(pyridin-4-yl)-1H-pyrazol-4-yl]-2-fluorophenyl}pyrrolidine-1-sulfonamide; rac-N-{5-chloro-3-[1-(4-{4-[(1-{4-[(3R)-2,6-dioxopiperidin-3-yl]-2,3-difluorophenyl}piperidin-4-yl)methyl]piperazin-1-yl}phenyl)-3-(pyridin-4-yl)-1H-pyrazol-4-yl]-2-fluorophenyl}pyrrolidine-1-sulfonamide; rac-N-{5-chloro-3-[1-(4-{4-[(1-{4-[(3R)-2,6-dioxopiperidin-3-yl]-2,5-difluorophenyl}piperidin-4-yl)methyl]piperazin-1-yl}phenyl)-3-(pyridin-4-yl)-1H-pyrazol-4-yl]-2-fluorophenyl}pyrrolidine-1-sulfonamide; rac-N-{5-chloro-3-[1-(4-{4-[(1-{4-[(3R)-2,6-dioxopiperidin-3-yl]-3,5-difluorophenyl}piperidin-4-yl)methyl]piperazin-1-yl}phenyl)-3-(pyridin-4-yl)-1H-pyrazol-4-yl]-2-fluorophenyl}pyrrolidine-1-sulfonamide; and N-[5-chloro-3-(1-{4-[4-({1-[5-(2,4-dioxo-1,3-diazinan-1-yl)pyridin-2-yl]piperidin-4-yl}methyl)piperazin-1-yl]phenyl}-3-(pyridin-4-yl)-1H-pyrazol-4-yl)-2-fluorophenyl]cyclopentanesulfonamide.

### Linkers

In Formula (I) or (II), L is a linker. The linker can be any linker suitable for linking the right and left portions of a molecule or Formula herein. In certain embodiments, the linker does not interfere with the harness or hook functions of a molecule or Formula herein. In certain embodiments, the linker provides useful solubility, flexibility, and/or distance between the portions of a molecule or Formula herein. In certain embodiments, L is a linker according to -L¹-L²-L³-L⁴-L⁵-L⁶-L⁷- or -L⁷-L⁶-L⁵-L⁴-L³-L²-L¹-. In certain embodiments, L is a linker according to -L¹-L²-L³-L⁴-L⁵-L⁶- or -L⁶-L⁵-L⁴-L³-L²-L¹-. In certain embodiments, L is a linker according to -L¹-L²-L³-L⁴-L⁵- or -L⁵-L⁴-L³-L²-L¹-. In certain embodiments, L is a linker according to -L¹-L²-L³-L⁴- or -L⁴-L³-L²-L¹-. In certain embodiments, L is a linker according to -L¹-L²-L³- or -L³-L²-L¹-. In certain embodiments, L is a linker according to -L¹-L²- or -L²-L¹-. In certain embodiments, L is a linker according to -L¹-, -L²-, -L³-, -L⁴-, -L⁵-, -L⁶-, -L⁷-, or other combinations as would be appreciated by a person of skill in the art. For example, in certain embodiments, L is a linker according to -L¹-L³-L⁶-, -L⁷-L⁵-L¹-, or -L¹-L³-L⁶-L²-. Each group L^{x} is described in detail below. In certain embodiments, the linker L comprises at least one heterocyclic group. In certain embodiments, the linker L comprises one heterocyclic group. In certain embodiments, the linker L comprises two heterocyclic groups. In certain embodiments, the linker L comprises three heterocyclic groups. In certain embodiments, the linker L comprises at least one spiro bicyclic heterocycloalkylene group. In certain embodiments, the linker L comprises one spiro bicyclic heterocycloalkylene group. In certain embodiments, the linker L comprises two spiro bicyclic heterocycloalkylene groups. In certain embodiments, the linker L comprises three spiro bicyclic heterocycloalkylene groups. In certain embodiments, the linker L comprises at least one heterocycloalkylene group and at least one spiro bicyclic heterocycloalkylene. The remaining groups of or within the linker are selected for chemical compatibility with adjacent groups, as will be recognized by those of skill in the art.

In certain embodiments, L is a linker according to -L¹-L²-L³-L⁴-L⁵-L⁶-L⁷-. In certain embodiments, L is a linker according to -L⁷-L⁶-L⁵-L⁴-L³-L²-L¹-. In certain embodiments, -L¹- is absent, —N(R¹⁰)—, —C(R¹¹)₂—, —C(O)—, —C₁₋₈ alkylene-, —C₂₋₈ alkynylene-, —C₆-C₁₀ aryl-, -heteroaryl-, —C₄-C₁₀ heteroaryl-, -Q¹-, or -Q²-; each -L²-, -L³-, -L⁴-, and -L⁵- is independently, absent, —N(R¹⁰)—, —C(R¹¹)₂—, —C(O)—, —O—, —(CH₂—CH₂—O)₁₋₈—, C₁₋₈ alkylene-, —C₂₋₈ alkynylene-, —C₆-C₁₀ aryl-, substituted —C₆-C₁₀ aryl-, -heteroaryl-, —C₄-C₁₀ heteroaryl-, -Q¹-, -Q²-, or -Q³-; each -L⁶- and -L⁷- is independently, absent, —N(R¹⁰)—, —C(R¹⁰)₂—, —C(O)—, —C(O)—N(R¹⁰)—, —N(R¹⁰)—C(O)—, -heteroaryl-, —C₄-C₁₀ heteroaryl-, or —C(R¹¹)₂—C(O)—N(R¹⁰)—. In certain embodiments, L comprises at least one -Q¹-. In certain embodiments, L comprises one -Q¹-. In certain embodiments, L comprises two -Q¹-. In certain embodiments, L comprises three -Q¹-. In certain embodiments, L comprises at least one -Q²-. In certain embodiments, L comprises one -Q²-. In certain embodiments, L comprises two -Q²-. In certain embodiments, L comprises three -Q²-. In certain embodiments, L comprises at least one -Q¹- and at least one -Q²-. In certain embodiments, L comprises one -Q¹- and one -Q²-;

In certain embodiments, each -Q¹- is an unsubstituted or substituted three- to seven-membered heterocycloalkylene comprising at least one nitrogen; each -Q²- is a five- to thirteen-membered bicyclic heterocycloalkylene comprising at least one nitrogen, wherein the five- to thirteen-membered bicyclic heterocycloalkylene is optionally a spiro bicyclic heterocycloalkylene ring; each -Q³- is a three- to six-membered cycloalkylene; each R¹⁰ is hydrogen or methyl; and each R¹¹ is independently, hydrogen, methyl, aryl, substituted aryl, or heteroaryl. In certain embodiments, R¹⁰ is hydrogen. In certain embodiments, R¹⁰ is methyl. In certain embodiments, one R¹¹ is hydrogen. In certain embodiments, both R¹¹ are hydrogen. In certain embodiments, one R¹¹ is methyl. In certain embodiments, one R¹¹ is hydrogen and the other is methyl. In certain embodiments, both R¹¹ are methyl. In certain embodiments, one R¹¹ is aryl. In certain embodiments, one R¹¹ is hydrogen or methyl and the other is aryl. In certain embodiments, both R¹¹ are aryl. In certain embodiments, R¹¹ is a substituted aryl. In certain embodiments, one R¹¹ is heteroaryl. In certain embodiments, one R¹¹ is hydrogen or methyl and the other is heteroaryl. In certain embodiments, both R¹¹ are heteroaryl.

In certain embodiments of Formula (I) or (II), L comprises at least one -Q¹-according to

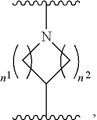

wherein n¹ is one or two, and n² is one or two.

In certain embodiments of Formula (I) or (II), L comprises at least one -Q¹-.

In certain embodiments of Formula (I) or (II), L is selected from

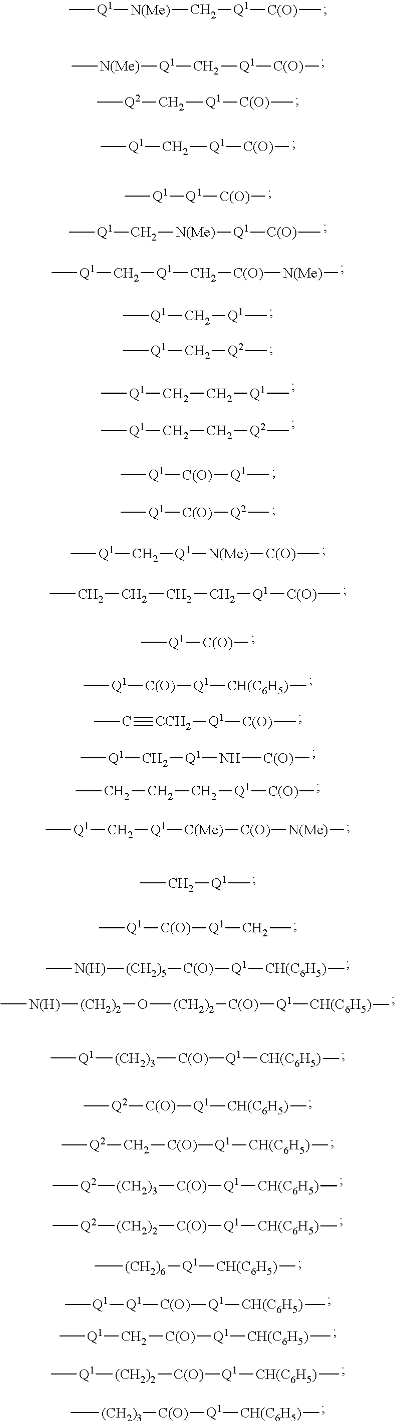

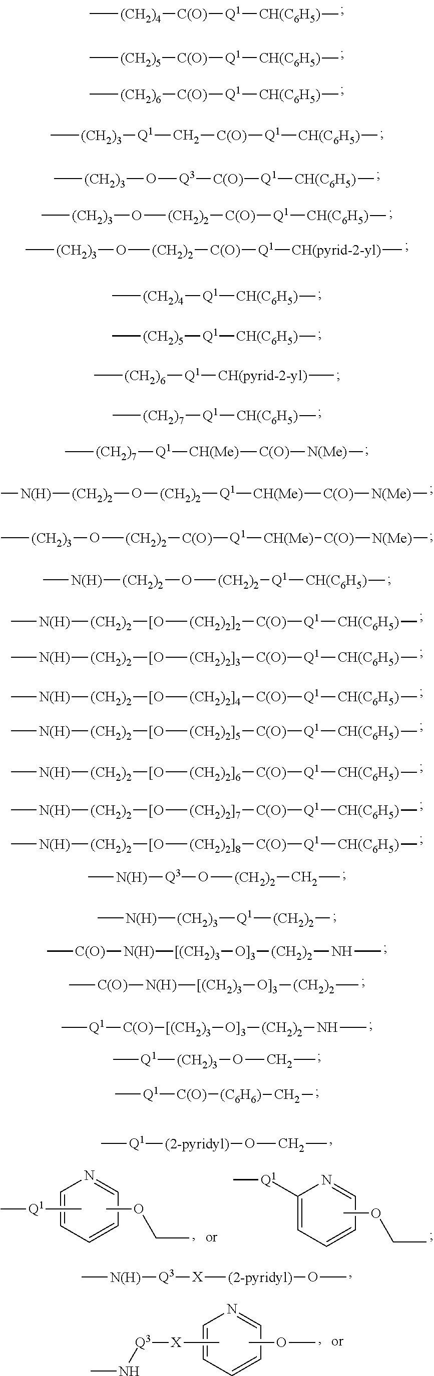

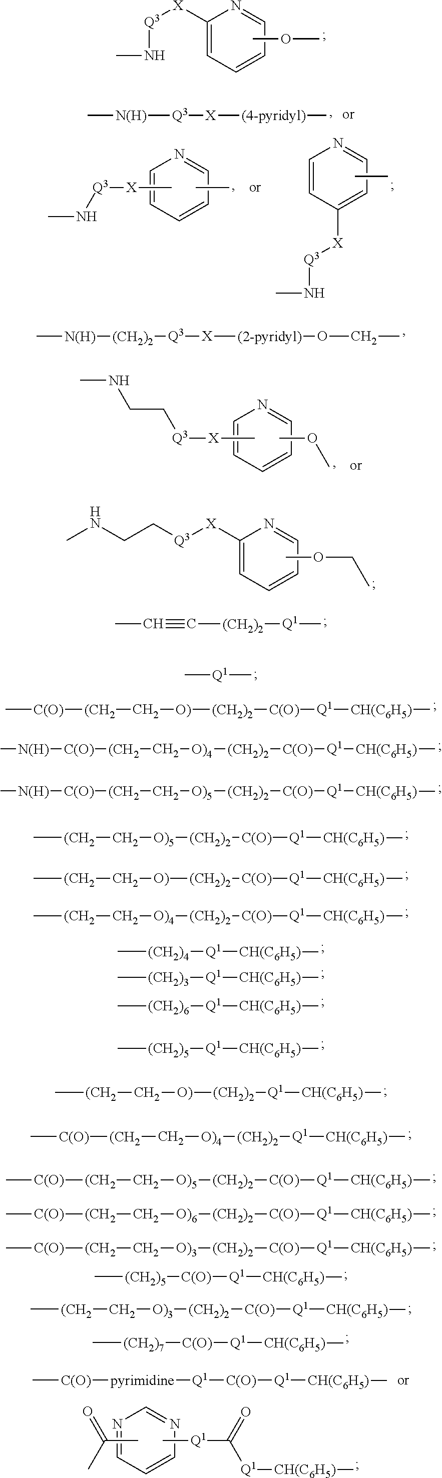

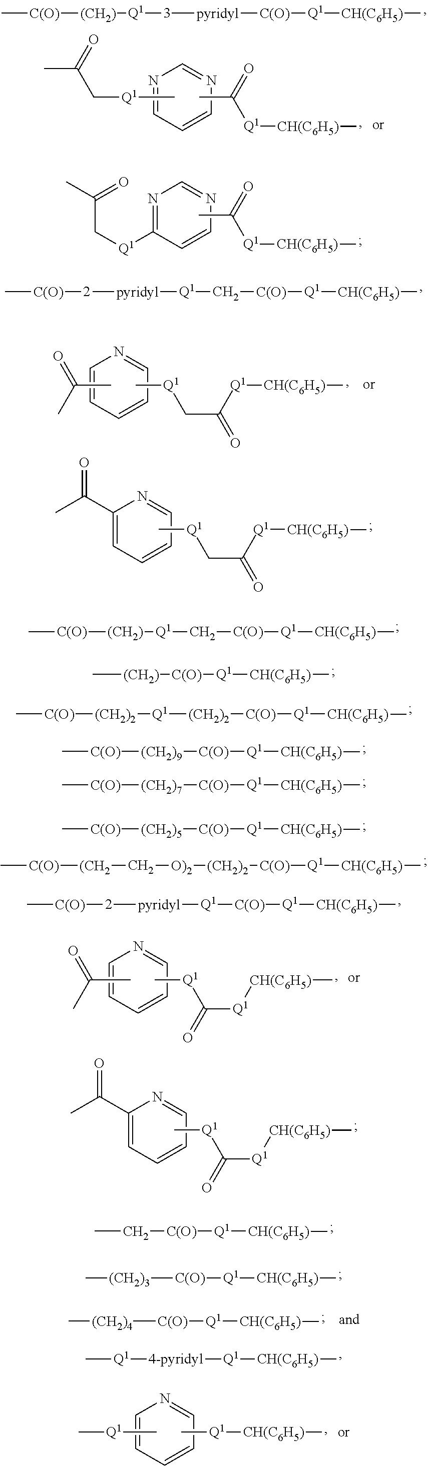

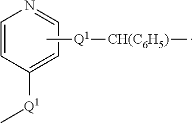

In some embodiments, X is oxygen or sulfur.

In certain embodiments of Formula (I) or Formula (II), L comprises at least one -Q¹- selected from the group consisting of

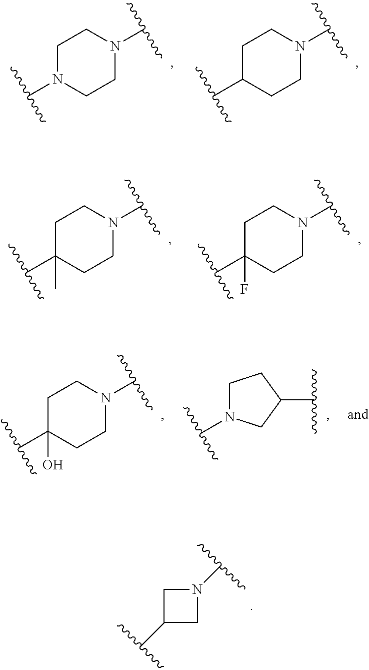

In certain embodiments of Formula (I) or Formula (II), L comprises at least one -Q¹- as

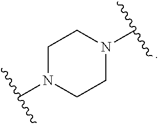 [SMILES: *N1CCN(*)CC1]

In certain embodiments of Formula (I) or Formula (II), L comprises at least one -Q¹- as

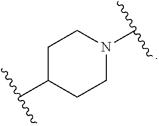 [SMILES: *C1CCN(*)CC1]

In certain embodiments of Formula (I) or Formula (II), L comprises at least one -Q¹- as

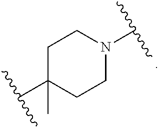 [SMILES: *N1CCC(*)(C)CC1]

In certain embodiments of Formula (I) or Formula (II), L comprises at least one -Q¹- as

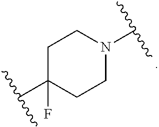 [SMILES: *N1CCC(*)(F)CC1]

In certain embodiments of Formula (I) or Formula (II), L comprises at least one -Q¹- as

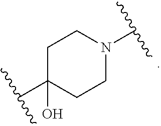 [SMILES: *N1CCC(*)(O)CC1]

In certain embodiments of Formula (I) or Formula (II), L comprises at least one -Q as

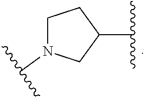 [SMILES: *C1CCN(*)C1]

In certain embodiments of Formula (I) or Formula (II), L comprises at least one -Q¹- as

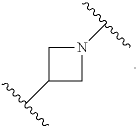 [SMILES: *C1CN(*)C1]

In certain embodiments of Formula (I) or Formula (II), L comprises at least one -Q²- according to

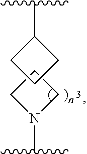

wherein n³ is one or two.

In certain embodiments of Formula (I) or Formula (II), L comprises at least one -Q²- according to

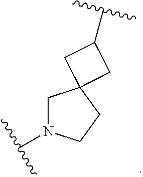 [SMILES: *C1CC2(CCN(*)C2)C1]

In certain embodiments of Formula (I) or Formula (II), L comprises at least one -Q²- according to

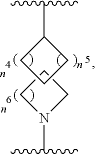

wherein n⁴ is one or two, n⁵ is one or two, and n⁶ is one or two.

In certain embodiments of Formula (I) or Formula (II), L comprises at least one -Q²- according to

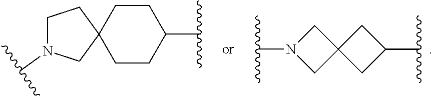 [SMILES: *C1CCC2(CC1)CCN(*)C2 ; *C1CC2(C1)CN(*)C2]

In certain embodiments of Formula (I) or Formula (II), L comprises at least one -Q²- according to

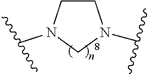

wherein n⁸ is one or two.

In certain embodiments of Formula (I) or Formula (II), L comprises at least one -Q²- according to

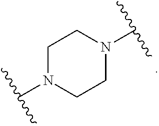 [SMILES: *N1CCN(*)CC1]

In certain embodiments of Formula (I) or Formula (II), L comprises at least one -Q²- according to

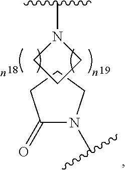

wherein n¹⁸ and n¹⁹ is two, or n¹⁸ is two and n¹⁹ is three, or n¹⁸ is three and n¹⁹ is two.

In certain embodiments of Formula (I) or Formula (II), L comprises at least one -Q²- according to

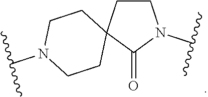 [SMILES: *N1CCC2(CC1)CCN(*)C2=O]

In certain embodiments of Formula (I) or Formula (II), L comprises at least one -Q²- according to

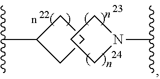

wherein n²² is zero to two; n²³ is zero to two, and n²⁴ is one or two, or wherein n²² is two and each n²³ and n²⁴ is one; or n²² is two and each n²³ and n²⁴ is two.

In certain embodiments of Formula (I) or Formula (II), L comprises at least one -Q²- according to

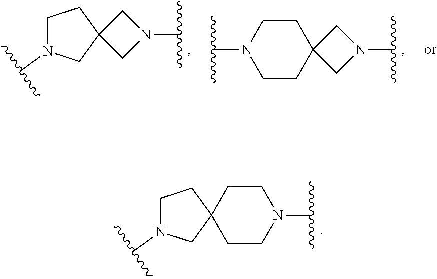

In certain embodiments of Formula (I) or Formula (II), L comprises at least one -Q²- according to

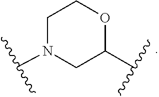 [SMILES: *C1CN(*)CCO1]

In certain embodiments of Formula (I) or Formula (II), L comprises at least one -Q³- according to

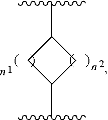

wherein n¹ is one or two, and n² is one or two.

In certain embodiments of Formula (I) or Formula (II), L comprises at least one -Q³- selected from the group consisting of

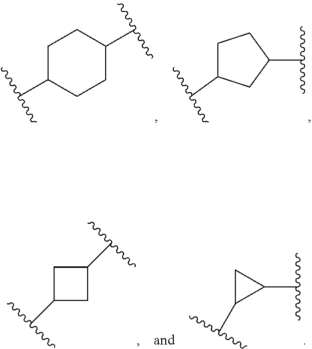 [SMILES: *C1CCC(*)CC1 ; *C1CCC(*)C1 ; *C1CC(*)C1 ; *C1CC1*]

In certain embodiments of Formula (I) or Formula (II), L comprises at least one -Q³- as

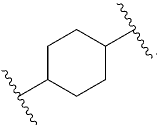 [SMILES: *C1CCC(*)CC1]

In certain embodiments of Formula (I) or Formula (II), L comprises at least one -Q³- as

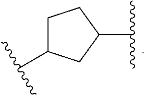 [SMILES: *C1CCC(*)C1]

In certain embodiments of Formula (I) or Formula (II), L comprises at least one -Q³- as

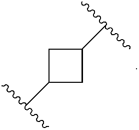 [SMILES: *C1CC(*)C1]

In certain embodiments of Formula (I) or Formula (II), L comprises at least one -Q³- as

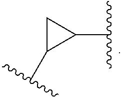 [SMILES: *C1CC1*]

### Pharmaceutical Compositions

The compounds described herein can be formulated into pharmaceutical compositions that further comprise a pharmaceutically acceptable carrier, diluent, adjuvant, or vehicle. In one embodiment, this disclosure provides a pharmaceutical composition comprising one or more compounds described herein, and a pharmaceutically acceptable carrier, diluent, adjuvant, or vehicle. In one embodiment, this disclosure provides a pharmaceutical composition comprising an effective amount of one or more compounds of this disclosure, or a pharmaceutically acceptable salt thereof and a pharmaceutically acceptable carrier, diluent, adjuvant, or vehicle. Pharmaceutically acceptable carriers include, for example, pharmaceutical diluents, excipients, or carriers suitably selected with respect to the intended form of administration, and consistent with conventional pharmaceutical practices.

According to another embodiment, this description provides a composition comprising one or more compounds herein, or a pharmaceutically acceptable salt thereof, and a pharmaceutically acceptable carrier, adjuvant, or vehicle. Pharmaceutical compositions of this description comprise a therapeutically effective amount of one or more compounds of Formula (I) or (II), wherein a “therapeutically effective amount” is an amount that is (a) effective to measurably degrade BRAF (or reduce the amount of BRAF) in a biological sample or in a patient; or (b) effective in treating and/or ameliorating a disease or disorder that is mediated by BRAF.

It will also be appreciated that certain compounds of this disclosure can exist in free form (e.g., a neutral compound) for treatment, or where appropriate, as a pharmaceutically acceptable derivative (e.g., a salt) thereof. According to this disclosure, a pharmaceutically acceptable derivative includes, but is not limited to, pharmaceutically acceptable prodrugs, salts, esters, salts of such esters, or any other adduct/educt or derivative that upon administration to a patient in need is capable of providing, directly or indirectly, one or more compounds as otherwise described herein, or a metabolite, or residue thereof.

As used herein, the term “pharmaceutically acceptable salt” refers to those salts that are, within the scope of sound medical practice or judgment, suitable for use in contact with cells, tissues, and/or the tissues of humans and lower animals without undue toxicity, irritation, allergic response, and the like.

Pharmaceutically acceptable salts are well known in the art. For example, S. M. Berge et al. describe pharmaceutically acceptable salts in detail in J. Pharmaceutical Sciences 1977, 66, 1-19, incorporated herein by reference. Pharmaceutically acceptable salts of the compounds of this description include those derived from suitable inorganic and organic acids and bases. Examples of pharmaceutically acceptable, nontoxic acid addition salts include salts of an amino group formed with inorganic acids such as hydrochloric acid, hydrobromic acid, phosphoric acid, sulfuric acid, and perchloric acid; or with organic acids such as acetic acid, oxalic acid, maleic acid, tartaric acid, citric acid, succinic acid, or malonic acid; or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, and valerate salts, and the like. Salts derived from appropriate bases include alkali metal, alkaline earth metal, ammonium (NH₄⁺), and N⁺(C₁₋₄ alkyl)₄ salts. This description also envisions the quaternization of any basic nitrogen-containing groups of the compounds disclosed herein. Water or oil-soluble or dispersable products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

A pharmaceutically acceptable carrier may contain inert ingredients that do not unduly inhibit the biological activity of the compounds described herein. The pharmaceutically acceptable carriers should be biocompatible, for example, non-toxic, non-inflammatory, non-immunogenic, and/or devoid of other undesired reactions or side-effects upon administration to a subject. Standard pharmaceutical formulation techniques can be employed.

The pharmaceutically acceptable carrier, adjuvant, or vehicle, as used herein, includes any and all solvents, diluents, or other liquid vehicle, dispersion, or suspension aids, surface active agents, isotonic agents, thickening or emulsifying agents, preservatives, solid binders, lubricants, and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions, and known techniques for the preparation thereof. Except insofar as any conventional carrier medium is incompatible with the compounds described herein, such as by producing any undesirable biological effect, or otherwise interacting in a deleterious manner with any other component(s) of the pharmaceutically acceptable composition, the use of such conventional carrier medium is contemplated to be within the scope of this description. As used herein, the phrase “side effects” encompasses unwanted and adverse effects of a therapy (e.g., a prophylactic or therapeutic agent). Side effects are usually unwanted, but unwanted effects are not necessarily adverse or unbeneficial. An adverse effect from a therapy (e.g., prophylactic or therapeutic agent) might be harmful, uncomfortable, or risky. Side effects include, but are not limited to, fever, chills, lethargy, gastrointestinal toxicities (including gastric and intestinal ulcerations and erosions), nausea, vomiting, neurotoxicities, nephrotoxicities or renal toxicities (including such conditions as papillary necrosis and chronic interstitial nephritis), hepatic toxicities (including elevated serum liver enzyme levels), myelotoxicities (including leukopenia, myelosuppression, thrombocytopenia, and anemia), dry mouth, metallic taste, prolongation of gestation, weakness, somnolence, pain (including muscle pain, bone pain, and headache), hair loss, asthenia, dizziness, extra-pyramidal symptoms, akathisia, cardiovascular disturbances, and sexual dysfunction.

Some examples of materials that can serve as pharmaceutically acceptable carriers include, but are not limited to, ion exchangers, alumina, aluminum stearate, lecithin, serum proteins (such as human serum albumin), buffer substances (such as Tween 80, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil, safflower oil, sesame oil, olive oil, corn oil, and soybean oil; glycols such as propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring, and perfuming agents. Preservatives and antioxidants can also be present in the composition, according to the judgment of the formulator.

As used herein, the term “measurably degrade” means a measurable reduction in (a) BRAF activity, between a sample comprising one or more compounds of this description and BRAF versus an equivalent sample comprising BRAF in the absence of said compound(s); or (b) the concentration of BRAF in a sample over time.

### Administration

The compositions of this disclosure may be administered orally, parenterally, via inhalation spray, topically, rectally, nasally, buccally, vaginally, or via an implanted reservoir. As used herein, the term “parenteral” includes subcutaneous, intravenous, intramuscular, intra-articular, intra-synovial, intrasternal, intrathecal, intraocular, intrahepatic, intralesional, and intracranial injection or infusion techniques. Compositions can be administered orally, intraperitoneally, or intravenously. Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives, are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans, and other emulsifying agents or bioavailability enhancers that are commonly used in the manufacture of pharmaceutically acceptable solid, liquid, or other dosage forms may also be used for the purposes of formulation.

The pharmaceutically acceptable compositions of this disclosure may be orally administered in any orally acceptable dosage form including, but not limited to, capsules, tablets, aqueous suspensions, or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring, or coloring agents may also be added.

Alternatively, the pharmaceutically acceptable compositions of this disclosure may be administered in the form of suppositories for rectal or vaginal administration. These can be prepared by mixing the agent with a suitable non-irritating excipient that is solid at room temperature but liquid at rectal or vaginal temperature and therefore will melt in the rectum or vaginal cavity to release the drug. Such materials include cocoa butter, polyethylene glycol, or a suppository wax that is solid at ambient temperature but liquid at body temperature and therefore melts in the rectum or vaginal cavity and releases the active compound.

The pharmaceutically acceptable compositions of this disclosure may also be administered topically, especially when the target of treatment includes areas or organs readily accessible by topical application, including diseases of the eye, skin, or lower intestinal tract. Suitable topical formulations are readily prepared for each of these areas or organs.

Topical application for the lower intestinal tract can be via a rectal suppository formulation (see above) or in a suitable enema formulation. Topically-transdermal patches may also be used.

For topical applications, the pharmaceutically acceptable compositions may be formulated in a suitable ointment containing the active component suspended or dissolved in one or more carriers. Carriers for topical administration of the compounds of this disclosure include, but are not limited to, mineral oil, liquid petrolatum, white petrolatum, propylene glycol, polyoxyethylene, polyoxypropylene compound, emulsifying wax, and water. Alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol, and water.

For ophthalmic use, the pharmaceutically acceptable compositions may be formulated, for example, as micronized suspensions in isotonic, pH adjusted sterile saline or other aqueous solution, or as solutions in isotonic, pH adjusted sterile saline or other aqueous solution, either with or without a preservative such as benzylalkonium chloride. Alternatively, for ophthalmic uses, the pharmaceutically acceptable compositions may be formulated in an ointment such as petrolatum. The pharmaceutically acceptable compositions of this disclosure may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In certain embodiments, the compositions of this disclosure are administered orally. The pharmaceutically acceptable compositions of this description may be orally administered in any orally acceptable dosage form including, but not limited to, capsules, tablets, aqueous suspensions, or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring, or coloring agents may also be added.

Liquid dosage forms for oral administration include, but are not limited to, pharmaceutically acceptable emulsions, microemulsions, solutions, suspensions, syrups, and elixirs. In addition to the biologically active compounds herein, the liquid dosage forms may contain inert diluents commonly used in the art, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid dosage forms for oral administration include capsules, tablets, pills, powders, and granules. In such solid dosage forms, the biologically active compound(s) described herein are mixed with at least one inert, pharmaceutically acceptable excipient or carrier such as sodium citrate or dicalcium phosphate, and/or a) fillers or extenders such as starches, lactose, sucrose, glucose, mannitol, and silicic acid; b) binders such as carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents, for example, cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. Solid dosage forms optionally may contain opacifying agents. These solid dosage forms can also be of a composition such that they release the active ingredient(s) only, for example, in a certain part of the intestinal tract, optionally in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds herein can also be in micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active compound may be admixed with at least one inert diluent such as sucrose, lactose, or starch. Such dosage forms may also comprise, as is normal practice, additional substances other than inert diluents, for example, tableting lubricants and other tableting aids such a magnesium stearate and microcrystalline cellulose. In the case of capsules, tablets, and pills, the dosage forms may also comprise buffering agents. They may optionally contain opacifying agents and can also be of a composition such that they release the active ingredient(s) only, for example, in a certain part of the intestinal tract, optionally in a delayed manner. Examples of embedding compositions that can be used include polymeric substances and waxes.

The compounds of the description are formulated in dosage unit form for ease of administration and uniformity of dosage. As used herein, the phrase “dosage unit form” refers to a physically discrete unit of agent appropriate for the subject, cell, tissue, or patient to be treated. It will be understood, however, that the total daily usage of the compounds, and compositions of this disclosure will be decided by the attending physician within the scope of sound medical judgment. The specific effective dose level for any particular subject, cell, tissue, patient, or organism will depend upon a variety of factors including the disorder being treated and the severity of the disorder; the activity of the specific compound(s) employed; the specific composition(s) employed; the age, body weight, general health, sex, and diet of the subject, cell, tissue, and/or patient; the time of administration, route of administration, and rate of excretion of the specific compound(s) employed; the duration of the treatment; drugs used in combination or coincidental with the specific compound(s) employed, and like factors well known in the medical arts.

The amount of the compounds of this disclosure that may be combined with the carrier materials to produce a composition in a single dosage form will vary depending upon the subject, cell, tissue, and/or host treated, the particular mode of administration, and other factors. The compositions should be formulated so that a dosage of between 0.01-100 mg/kg body weight/day of the compound(s) or inhibitor(s) can be administered to a subject, cell, tissue, and/or patient receiving these compositions.

Depending upon the particular condition or disease to be treated or prevented, additional therapeutic agents, which are normally administered to treat or prevent that condition, may also be present in the compositions of this disclosure. As used herein, additional therapeutic agents that are normally administered to treat or prevent a particular disease, or condition, are known as “appropriate for the disease, or condition, being treated.”

The compound or composition can be administered concurrently with, prior to, or subsequent to, one or more additional therapeutically active agents. In general, each agent will be administered at a dose and/or on a time schedule determined for that agent. It will further be appreciated that the additional therapeutically active agent utilized in this combination can be administered together in a single composition or administered separately in different composition. The particular combination to employ in a regimen will take into account compatibility of the compounds described herein with the additional therapeutically active agent and/or the desired therapeutic effect to be achieved. In general, it is expected that additional therapeutically active agents utilized in combination will be utilized at levels that do not exceed the levels at which they are utilized individually. In some embodiments, the levels utilized in combination will be lower than those utilized individually. Additional therapeutically active agents include, but are not limited to, small organic molecules such as drug compounds (e.g., compounds approved by the Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional therapeutically active agent is a cancer agent (e.g., a biotherapeutic or chemo therapeutic cancer agent). In other embodiments, the additional therapeutically active agent is an anti-inflammatory agent.

The amount of additional therapeutic agent present in the compositions of this disclosure will be no more than the amount that would normally be administered in a composition comprising that therapeutic agent as the only active agent. The amount of additional therapeutic agent in the presently disclosed compositions will range from about 50% to 100% of the amount normally present in a composition comprising that agent as the only therapeutically active agent.

### Methods of Use

The bifunctional compounds described herein are useful for degrading BRAF in biological samples, or in patients via a ubiquitin proteolytic pathway. Thus, an embodiment of this disclosure provides a method of treating a BRAF-mediated disease or disorder. As used herein, the term “BRAF-mediated disease or disorder” means any disease, disorder, or other deleterious condition in which BRAF is known to play a role. In some instances, an BRAF-mediated disease or disorder is a proliferative disorder or an autoimmune disorder. Examples of proliferative disorders include cancer.

In one aspect, provided herein are methods of treating or preventing cancer in a subject in need thereof. In certain embodiments, the methods comprise the step of orally administering to the subject an amount of a bifunctional compounds(s) described herein capable of inducing proteolytic degradation of BRAF. In certain embodiments, the amount is effective to treat or prevent the cancer.

In certain embodiments, the cancer is any cancer described below. In particular embodiments, the cancer comprises a solid tumor. In certain embodiments, the cancer is a B cell malignancy. In certain embodiments, the cancer is selected from the group consisting of chronic lymphocytic leukemia (CLL), small lymphocytic lymphoma (SLL), transformed CLL or Richter's transformation, small cell lymphoma, follicular lymphoma (FL), diffuse large B-cell lymphoma (DLBCL), non-Hodgkin lymphoma, mantle cell lymphoma (MCL), marginal zone lymphoma (MZL), Waldenstrom macroglobulinemia (WM), and central nervous system (CNS) lymphoma. In certain embodiments, the cancer is chronic lymphocytic leukemia. In certain embodiments, the cancer is small cell lymphoma. In certain embodiments, the cancer is follicular lymphoma. In certain embodiments, the cancer is diffuse large B-cell lymphoma. In certain embodiments, the cancer is non-Hodgkin lymphoma. In certain embodiments, the cancer is mantle cell lymphoma. In certain embodiments, the cancer is marginal zone lymphoma. In certain embodiments, the cancer is Waldenstrom macroglobulinemia. In certain embodiments, the cancer is small lymphocytic lymphoma (SLL). In certain embodiments, the cancer is CNS lymphoma. In certain embodiments, the cancer is transformed CLL or Richter's transformation. In certain embodiments, the cancer is chronic lymphocytic leukemia (CLL).

In another aspect, provided herein are methods of degrading BRAF in a subject in need thereof. The methods comprise the step of orally administering to the subject an amount of a bifunctional compound(s) described herein and capable of inducing proteolytic degradation of BRAF. In certain embodiments, the amount is effective to degrade BRAF in the subject. The BRAF can be expressed in any cells or tissues of the subject.

In another aspect, provided herein are methods of preventing B cell activation in a subject in need thereof. The methods comprise the step of orally administering to the subject an amount of a bifunctional compound(s) described herein and capable of inducing proteolytic degradation of BRAF. In certain embodiments, the amount is effective to prevent B cell activation. In certain embodiments, the B cell expresses CD69. In certain embodiments, the B cell expresses CD86. In certain embodiments, the B cell expresses CD69 and CD86.

In the methods, the bifunctional compound(s) described herein comprise a moiety capable of specifically binding BRAF and further comprise a moiety capable of recruiting a ubiquitin ligase to degrade the BRAF. Particular compounds with each capability are described herein. The compounds can be administered in any form, including pharmaceutically acceptable salts and pharmaceutical compositions.

The bifunctional compound(s) described herein can be administered in any dose deemed suitable by the practitioner of skill. In certain embodiments, the dose is 0.1-1000 mg/kg. In certain embodiments, the dose is 0.1-900 mg/kg. In certain embodiments, the dose is 0.1-800 mg/kg. In certain embodiments, the dose is 0.1-700 mg/kg. In certain embodiments, the dose is 0.1-600 mg/kg. In certain embodiments, the dose is 0.1-500 mg/kg. In certain embodiments, the dose is 0.1-400 mg/kg. In certain embodiments, the dose is 0.1-300 mg/kg. In certain embodiments, the dose is 0.1-200 mg/kg. In certain embodiments, the dose is 0.1-100 mg/kg. In certain embodiments, the dose is selected from the group consisting of 100 mg/kg, 200 mg/kg, 300 mg/kg, 450 mg/kg, 600 mg/kg, 800 mg/kg, and 1000 mg/kg. In certain embodiments, the dose is about 25 mg/kg. In certain embodiments, the dose is about 50 mg/kg. In certain embodiments, the dose is about 75 mg/kg. In certain embodiments, the dose is about 100 mg/kg. In certain embodiments, the dose is about 150 mg/kg. In certain embodiments, the dose is about 200 mg/kg. In certain embodiments, the dose is about 250 mg/kg. In certain embodiments, the dose is about 300 mg/kg. In certain embodiments, the dose is about 400 mg/kg. In certain embodiments, the dose is about 450 mg/kg. In certain embodiments, the dose is about 500 mg/kg. In certain embodiments, the dose is about 600 mg/kg. In certain embodiments, the dose is about 700 mg/kg. In certain embodiments, the dose is about 750 mg/kg. In certain embodiments, the dose is about 800 mg/kg. In certain embodiments, the dose is about 900 mg/kg. In certain embodiments, the dose is about 1000 mg/kg.

The dose can be administered on a schedule deemed suitable by the person of skill in the art. In certain embodiments, the dose is administered once per day. In certain embodiments, the dose is administered twice per day. In certain embodiments, the dose is administered three times per day. In certain embodiments, the dose is administered four times per day. In certain embodiments, the dose is administered in divided doses. In certain embodiments, the dose is administered in two divided doses per day. In certain embodiments, the dose is administered in three divided doses per day. In certain embodiments, the dose is administered in four divided doses per day.

Dosing can continue for any length of time deemed suitable by the person of skill in the art. In certain embodiments, the dose is administered daily for fourteen days. In certain embodiments, the dose is administered daily for thirteen days. In certain embodiments, the dose is administered daily for twelve days. In certain embodiments, the dose is administered daily for eleven days. In certain embodiments, the dose is administered daily for ten days. In certain embodiments, the dose is administered daily for nine days. In certain embodiments, the dose is administered daily for eight days. In certain embodiments, the dose is administered daily for seven days. In certain embodiments, the dose is administered daily for six days. In certain embodiments, the dose is administered daily for five days. In certain embodiments, the dose is administered daily for four days. In certain embodiments, the dose is administered daily for three days. In certain embodiments, the dose is administered daily for two days. In certain embodiments, the dose is administered for one day.

In the dosing schedule, the doses can be administered on consecutive days or cyclically, according to the judgment of the practitioner of skill. In certain embodiments, the doses are administered on consecutive days. In certain embodiments, the doses are administered with an interval between doses. In certain embodiments, the interval is one day. In certain embodiments, the interval is two days. In certain embodiments, the interval is three days. In certain embodiments, the interval is four days. In certain embodiments, the interval is five days. In certain embodiments, the interval is six days.

In certain embodiments, the dose is administered weekly. In certain embodiments, the dose is administered twice per week. In certain embodiments, the dose is administered three times per week.

In certain embodiments, the dose(s) are administered for a period of time with a first interval between dose(s), and then the dose(s) are re-administered for a period of time following the first interval between dose(s), wherein this dosing regimen can be repeated (i.e., cyclically or cyclically, for example, after a second, third, etc. interval between subsequent administrations of dose(s)) according to the judgment of the practitioner of skill. For example, in one embodiment, a first dose is administered for one week, followed by a first interval of one week without the first dose administration; then, a second dose is re-administered for another week, followed by a second interval of one week without the first or second dose administration, and so on cyclically. Other perturbations for first, second, third, etc. dose(s) followed by perturbations for first, second, third, etc. interval(s), and combinations thereof, are contemplated herein as would be appreciated by the practitioner of skill and the need of the subject, cell, tissue, and/or patient. For example, in one embodiment, a first dose is administered daily for one week, followed by a first interval of three weeks without the first daily dose administration; then, a second dose is re-administered biweekly for another week, followed by a second interval of four weeks without the first daily or second biweekly dose administration, and so on cyclically.

The compound can be administered by any route of administration deemed suitable by the practioner of skill. In certain embodiments, the dose is administered orally. Formulations and techniques for administration are described elsewhere herein.

In certain embodiments, term “cancer” includes, but is not limited to, the following cancers: epidermoid Oral: buccal cavity, lip, tongue, mouth, pharynx, squamous cell carcinoma of the head and neck (HNSCC); Cardiac: sarcoma (angiosarcoma, fibrosarcoma, rhabdomyosarcoma, liposarcoma), myxoma, rhabdomyoma, fibroma, lipoma, and teratoma; Lung: bronchogenic carcinoma (squamous cell or epidermoid, undifferentiated small cell, undifferentiated large cell, adenocarcinoma), alveolar (bronchiolar) carcinoma, bronchial adenoma, sarcoma, lymphoma, chondromatous hamartoma, mesothelioma, non-small cell lung cancer (NSCLC); Gastrointestinal: gastric cancer, esophagus (squamous cell carcinoma, larynx, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel or small intestines (adenocarcinoma, lymphoma, carcinoid tumors, Karposi's sarcoma, leiomyoma, hemangioma, lipoma, neurofibroma, fibroma), large bowel or large intestines (adenocarcinoma, tubular adenoma, villous adenoma, hamartoma, leiomyoma), colon, colon-rectum, colorectal, microsatellite stable colorectal cancer (MSS CRC), rectum; Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma), metastatic castrate-resistant prostate cancer (mCRPC), muscle-invasive urothelial cancer; Liver: hepatoma (hepatocellular carcinoma), cholangiocarcinoma, hepatoblastoma, angiosarcoma, hepatocellular adenoma, hemangioma, biliary passages; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma (MM), malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical cancer, cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), breast, triple-negative breast cancer (TNBC), platinum-resistant epithelial ovarian cancer (EOC); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma) hairy cell; lymphoid disorders (e.g., mantle cell lymphoma, Waldenstrom's macroglobulinemia, Marginal zone lymphoma, and Follicular lymphoma); Skin: malilymphgnant melanoma, basal cell carcinoma, squamous cell carcinoma, Karposi's sarcoma, keratoacanthoma, moles or dysplastic nevi, lipoma, angioma, dermatofibroma, keloids, psoriasis; Thyroid gland: papillary thyroid carcinoma, follicular thyroid carcinoma; medullary thyroid carcinoma, undifferentiated thyroid cancer, multiple endocrine neoplasia type 2A, multiple endocrine neoplasia type 2B, familial medullary thyroid cancer, pheochromocytoma, paraganglioma; Adrenal glands: neuroblastoma; and metastatic melanoma.

In certain embodiments, the term “autoimmune disease” includes, but is not limited to, the following autoimmune diseases: uticaria, graft-versus-host disease (GVHD), acute graft-versus-host disease, pemphigus vulgaris, achalasia, Addison's disease, Adult Still's disease, agammaglobulinemia, alopecia areata, amyloidosis, ankylosing spondylitis, anti-GBM/anti-TBM nephritis, antiphospholipid syndrome, autoimmune angioedema, autoimmune dysautonomia, autoimmune encephalomyelitis, autoimmune hepatitis, autoimmune inner ear disease (AIED), autoimmune myocarditis, autoimmune oophoritis, autoimmune orchitis, autoimmune pancreatitis, autoimmune retinopathy, axonal and neuronal neuropathy (AMAN), Baló disease, Behcet's disease, benign mucosal pemphigoid, bullous pemphigoid, Castleman disease (CD), Celiac disease, Chagas disease, chronic inflammatory demyelinating polyneuropathy (CIDP), chronic recurrent multifocal osteomyelitis (CRMO), Churg-Strauss Syndrome (CSS) or Eosinophilic Granulomatosis (EGPA), cicatricial pemphigoid, Cogan's syndrome, cold agglutinin disease, congenital heart block, coxsackie myocarditis, CREST syndrome, Crohn's disease, dermatitis herpetiformis, dermatomyositis, Devic's disease (neuromyelitis optica), discoid lupus, Dressler's syndrome, endometriosis, eosinophilic esophagitis (EoE), eosinophilic fasciitis, erythema nodosum, essential mixed cryoglobulinemia, Evans syndrome, fibromyalgia, fibrosing alveolitis, giant cell arteritis (temporal arteritis), giant cell myocarditis, glomerulonephritis, Goodpasture's syndrome, granulomatosis with polyangiitis, Graves' disease, Guillain-Barre syndrome, Hashimoto's thyroiditis, hemolytic anemia, Henoch-Schonlein purpura (HSP), herpes gestationis or pemphigoid gestationis (PG), hidradenitis suppurativa (HS) (Acne Inversa), hypogammaglobulinemia, IgA nephropathy, IgG4-related sclerosing disease, immune thrombocytopenic purpura (ITP), inclusion body myositis (IBM), interstitial cystitis (IC), juvenile arthritis, juvenile diabetes (Type 1 diabetes), juvenile myositis (JM), Kawasaki disease, Lambert-Eaton syndrome, leukocytoclastic vasculitis, lichen planus, lichen sclerosus, ligneous conjunctivitis, linear IgA disease (LAD), lupus, lyme disease (chronic), Meniere's disease, microscopic polyangiitis (MPA), mixed connective tissue disease (MCTD), Mooren's ulcer, Mucha-Habermann disease, Multifocal Motor Neuropathy (MMN) or MMNCB, multiple sclerosis, myasthenia gravis, myositis, narcolepsy, neonatal lupus, neuromyelitis optica, neutropenia, ocular cicatricial pemphigoid, optic neuritis, palindromic rheumatism (PR), PANDAS, paraneoplastic cerebellar degeneration (PCD), paroxysmal nocturnal hemoglobinuria (PNH), Parry Romberg syndrome, pars planitis (peripheral uveitis), Parsonnage-Turner syndrome, pemphigus, peripheral neuropathy, perivenous encephalomyelitis, pernicious anemia (PA), POEMS syndrome, polyarteritis nodosa, polyglandular syndromes type I, II, or III, polymyalgia rheumatica, polymyositis, postmyocardial infarction syndrome, postpericardiotomy syndrome, primary biliary cirrhosis, primary sclerosing cholangitis, progesterone dermatitis, psoriasis, psoriatic arthritis, pure red cell aplasia (PRCA), pyoderma gangrenosum, Raynaud's phenomenon, reactive arthritis, reflex sympathetic dystrophy, relapsing polychondritis, restless legs syndrome (RLS), retroperitoneal fibrosis, rheumatic fever, rheumatoid arthritis, sarcoidosis, Schmidt syndrome, scleritis, scleroderma, Sjögren's syndrome, sperm and testicular autoimmunity, stiff person syndrome (SPS), subacute bacterial endocarditis (SBE), Susac's syndrome, sympathetic ophthalmia (SO), Takayasu's arteritis, temporal arteritis (giant cell arteritis), thrombocytopenic purpura (TTP), Tolosa-Hunt syndrome (THS), transverse myelitis, Type 1 diabetes, ulcerative colitis (UC), undifferentiated connective tissue disease (UCTD), uveitis, vasculitis, vitiligo, Vogt-Koyanagi-Harada Disease, and Wegener's granulomatosis (or Granulomatosis with Polyangiitis (GPA)).

In certain embodiments, the term “inflammatory disease” includes, but is not limited to, the following inflammatory diseases: encephalitis, myelitis, meningitis, arachnoiditis, neuritis, dacryoadenitis, scleritis, episcleritis, keratitis, retinitis, chorioretinitis, blepharitis, conjunctivitis, uveitis, otitisexterna, otitismedia, labyrinthitis, mastoiditis, endocarditis, myocarditis, pericarditis, vasculitis, arteritis, phlebitis, capillaritis, sinusitis, rhinitis, pharyngitis, laryngitis, tracheitis, bronchitis, bronchiolitis, pneumonitis, pleuritis, mediastinitis, stomatitis, gingivitis, gingivostomatitis, glossitis, tonsillitis, sialadenitis/parotitis, cheilitis, pulpitis, gnathitis, esophagitis, gastritis, gastroenteritis, enteritis, colitis, enterocolitis, duodenitis, ileitis, caecitis, appendicitis, proctitis, hepatitis, ascending cholangitis, cholecystitis, pancreatitis, peritonitis, dermatitis, folliculitis, cellulitis, hidradenitis, arthritis, dermatomyositis, myositis, synovitis/tenosynovitis, bursitis, enthesitis, fasciitis, capsulitis, epicondylitis, tendinitis, panniculitis, osteochondritis/osteitis/osteomyelitis, spondylitis, periostitis, chondritis, nephritis, glomerulonephritis, pyelonephritis, urethritis, cystitis, urethritis, oophoritis, salpingitis, endometritis, parametritis, cervicitis, vaginitis, vulvitis, mastitis, orchitis, epididymitis, prostatitis, seminalvesiculitis, balanitis, posthitis, balanoposthitis, chorioamnionitis, funisitis, omphalitis, insulitis, hypophysitis, thyroiditis, parathyroiditis, adrenalitis, lymphangitis, and lymphadenitis.

### Articles of Manufacture and Kits

Also provided are articles of manufacture comprising any of the compounds or pharmaceutical compositions described herein. The articles of manufacture include suitable containers or packaging materials for the compounds or pharmaceutical compositions. Examples of a suitable container include, but are not limited to, a bottle, a vial, a syringe, an intravenous bag, or a tube.

Also provided are kits comprising any of the compounds or pharmaceutical compositions described herein. The kits can contain the compounds or pharmaceutical compositions in suitable containers or packaging materials, including, but not limited to, a bottle, a vial, a syringe, an intravenous bag, or a tube. The kits can comprise the compounds or pharmaceutical compositions for administration to a subject, cell, tissue, and/or individual in single-dose form or in multiple-dose form. The kits can further comprise instructions or a label for administering the compounds or pharmaceutical compositions to a subject, cell, tissue, and/or individual according to any of the methods disclosed herein. The kits can further comprise equipment for administering the compounds or pharmaceutical compositions to a subject, cell, tissue, and/or individual, including, but not limited to, needles, syringes, tubing, or intravenous bags. The kits can further comprise instructions for producing any of the compounds or pharmaceutical compositions disclosed herein.

Also provided are articles of manufacture comprising any of the compounds or pharmaceutical compositions described herein. The articles of manufacture include suitable containers or packaging materials for the compounds or pharmaceutical compositions. The articles of manufacture include suitable containers or packaging materials for the compounds or pharmaceutical compositions. Examples of a suitable container include, but are not limited to, a bottle, a vial, a syringe, an intravenous bag, or a tube.

This disclosure will be more fully understood by reference to the following Examples. They should not, however, be construed as limiting the scope of this disclosure. It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

Additional embodiments are disclosed in further detail in the following examples, which are not in any way intended to limit the scope of the claims.

### Analytical Methods and Instrumentation

Proton nuclear magnetic resonance (NMR) spectra were obtained on Bruker Ascend™ 500 MHz spectrometer. NMR spectra are reported as follows: chemical shift δ (ppm), multiplicity, coupling constant J(Hz), and (relative) integration. The abbreviations s=singlet, d=doublet, t=triplet, q=quartet, m=multiplet, and br=broad are used throughout. Mass spectral data were measured using the following systems: Waters Acquity i-class ultra-performance liquid chromatography (UPLC) system with Acquity Photo Diode Array Detector, Acquity Evaporative Light Scattering Detector (ELSD), and Waters ZQ Mass Spectrometer. Data was acquired using Waters MassLynx 4.1 software and purity was characterized by UV wavelength 220 nm, evaporative light scattering detection (ELSD), and electrospray positive ion (ESI) (column: Acquity UPLC BEH C18 1.7 μ t 2.1×50 mm). Solvents used: acetonitrile/water, containing 0.1% formic acid; flow rate 0.7 mL/min. Preparatory HPLC purifications were conducted with a flow rate of 15 mL/min and detection by UV wavelength at 214 nm and 254 nm (Column: Jupiter© 10 μM Proteo 90 Å, 250×21.2 mm A, solvent: acetonitrile/water, containing a modifier such as 0.1% trifluoroacetic acid).

Abbreviations used in the examples include:

Lengthy table referenced here
US20260317324A1-20261015-T00001
Please refer to the end of the specification for access instructions.

Lengthy table referenced here
US20260317324A1-20261015-T00002
Please refer to the end of the specification for access instructions.

### Synthesis of N-{2-[5-(2-aminopyrimidin-4-yl)-4-[2-fluoro-3-(propane-1-sulfonamido)phenyl]-1,3-thiazol-2-yl]-2-methylpropyl}oxane-4-carboxamide

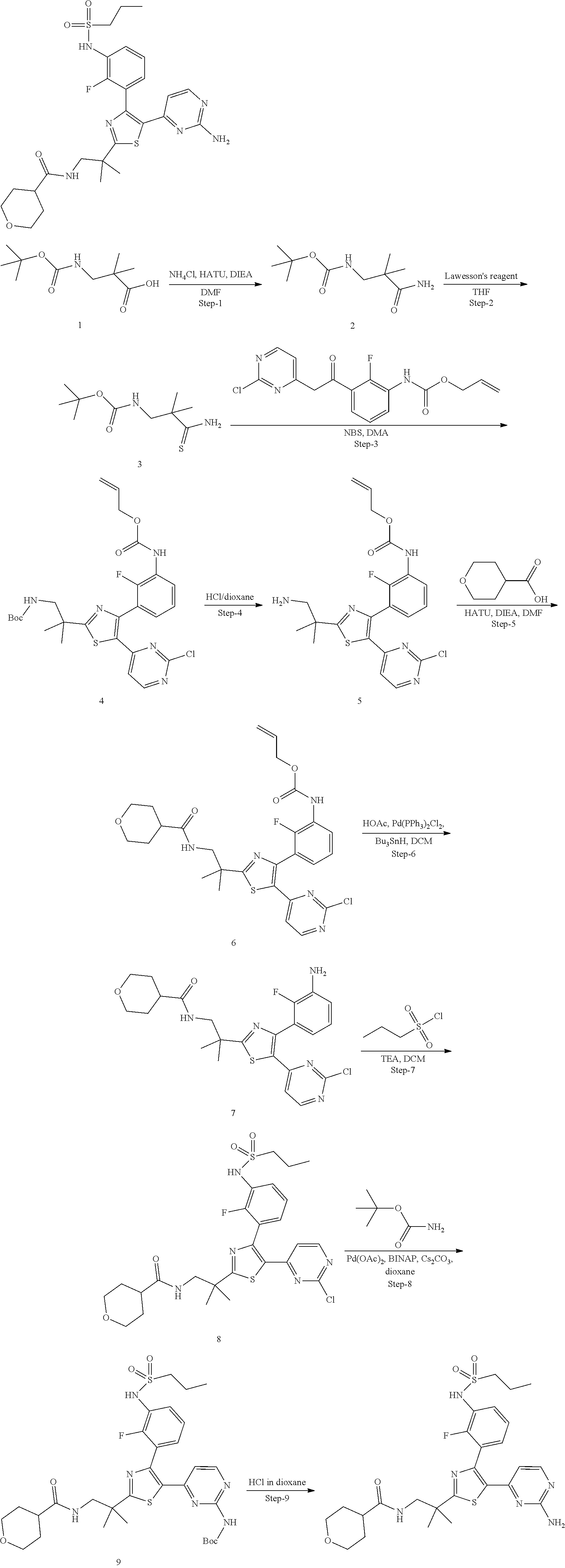

### Step-1: Synthesis of tert-butyl N-(2-carbamoyl-2,2-dimethylethyl)carbamate (2)

To a mixture of 3-[(tert-butoxycarbonyl)amino]-2,2-dimethylpropanoic acid (5 g, 23.01 mmol, 1 equiv) and NH₄Cl (6.15 g, 115.06 mmol, 5 equiv) in DMF (50 mL) was added HATU (13.13 g, 34.52 mmol, 1.5 equiv) and DIEA (8.92 g, 69.03 mmol, 3 equiv) at room temperature. The resulting mixture was stirred for one hour at room temperature. The resulting mixture was extracted with EtOAc. The combined organic layers were washed with water and dried over anhydrous Na₂SO₄. After filtration, the filtrate was concentrated under reduced pressure to afford tert-butyl N-(2-carbamoyl-2,2-dimethylethyl)carbamate (3.5 g, crude) as a yellow oil.

### Step-2: Synthesis of tert-butyl N-(2-carbamothioyl-2,2-dimethylethyl)carbamate (3)

To a mixture of tert-butyl N-(2-carbamoyl-2,2-dimethylethyl)carbamate (3.5 g, 16.183 mmol, 1 equiv) in THF (40 mL) was added Lawesson's Reagent (3.27 g, 8.091 mmol, 0.5 equiv) at room temperature. The resulting mixture was stirred overnight at room temperature. The resulting mixture was extracted with EtOAc. The combined organic layers were washed with water and dried over anhydrous Na₂SO₄. After filtration, the filtrate was concentrated under reduced pressure. The residue was purified by silica gel column chromatography, eluted with PE:EtOAc(5:1) to afford tert-butyl N-(2-carbamothioyl-2,2-dimethylethyl)carbamate (1.6 g, 38.30%) as a white solid. LCMS: C₁₀H₂₀N₂O₂S requires: 232.1, found: m/z=233.1 [M+H]⁺.

### Step-3: Synthesis of tert-butyl N-{2-[5-(2-chloropyrimidin-4-yl)-4-(2-fluoro-3-{[(prop-2-en-1-yloxy)carbonyl]amino}phenyl)-1,3-thiazol-2-yl]-2-methylpropyl}carbamate (4)

To a mixture of tert-butyl N-(2-carbamothioyl-2,2-dimethylethyl)carbamate (370 mg, 1.59 mmol, 1 equiv) and NBS (283 mg, 1.59 mmol, 1 equiv) in DMA (5 mL) was added prop-2-en-1-yl N-{3-[2-(2-chloropyrimidin-4-yl)acetyl]-2-fluorophenyl}carbamate (556 mg, 1.59 mmol, 1 equiv) at room temperature. The resulting mixture was stirred for 2 h at 60° C. The resulting mixture was extracted with EtOAc. The combined organic layers were washed with water and dried over anhydrous Na₂SO₄. After filtration, the filtrate was concentrated under reduced pressure. The residue was purified by silica gel column chromatography, eluted with PE:EtOAc (5:1) to afford tert-butyl N-{2-[5-(2-chloropyrimidin-4-yl)-4-(2-fluoro-3-{[(prop-2-en-1-yloxy)carbonyl]amino}phenyl)-1,3-thiazol-2-yl]-2-methylpropyl}carbamate (200 mg, 20.11%) as a white solid. LCMS: C₂₆H₂₉ClFN₅O₄S requires: 561.2, found: m/z=562.1 [M+H]⁺.

### Step-4: Synthesis of prop-2-en-1-yl N-{3-[2-(1-amino-2-methylpropan-2-yl)-5-(2-chloropyrimidin-4-yl)-1,3-thiazol-4-yl]-2-fluorophenyl}carbamate (5)

A solution of tert-butyl N-{2-[5-(2-chloropyrimidin-4-yl)-4-(2-fluoro-3-{[(prop-2-en-1-yloxy)carbonyl]amino}phenyl)-1,3-thiazol-2-yl]-2-methylpropyl}carbamate (190 mg, 0.338 mmol, 1 equiv) in HCl/1,4-dioxane (5 mL, 4 M) was stirred for one hour at room temperature. The resulting mixture was concentrated under reduced pressure to afford prop-2-en-1-yl N-{3-[2-(1-amino-2-methylpropan-2-yl)-5-(2-chloropyrimidin-4-yl)-1,3-thiazol-4-yl]-2-fluorophenyl}carbamate (110 mg, crude) as a yellow oil. LCMS: C₂₁H₂₁ClFN₅O₂S requires: 461.1, found: m/z=462.0 [M+H]⁺.

### Step-5: Synthesis of prop-2-en-1-yl N-{3-[5-(2-chloropyrimidin-4-yl)-2-[2-methyl-1-(oxan-4-ylformamido)propan-2-yl]-1,3-thiazol-4-yl]-2-fluorophenyl}carbamate (6)

To a mixture of prop-2-en-1-yl N-{3-[2-(1-amino-2-methylpropan-2-yl)-5-(2-chloropyrimidin-4-yl)-1,3-thiazol-4-yl]-2-fluorophenyl}carbamate (200 mg, 0.43 mmol, 1 equiv) and oxane-4-carboxylic acid (84 mg, 0.64 mmol, 1.5 equiv) in DMF (2 mL) was added HATU (246 mg, 0.64 mmol, 1.5 equiv) and DIEA (279 mg, 2.16 mmol, 5 equiv) at room temperature. The resulting mixture was stirred for one hour at room temperature. The resulting mixture was extracted with EtOAc. The combined organic layers were washed with water and dried over anhydrous Na₂SO₄. After filtration, the filtrate was concentrated under reduced pressure. The residue was purified by silica gel column chromatography, eluted with PE:EtOAc (5:1) to afford prop-2-en-1-yl N-{3-[5-(2-chloropyrimidin-4-yl)-2-[2-methyl-1-(oxan-4-ylformamido)propan-2-yl]-1,3-thiazol-4-yl]-2-fluorophenyl}carbamate (150 mg, 53.11%) as a yellow oil. LCMS: C₂₇H₂₉ClFN₅O₄S requires: 573.2, found: m/z=574.1 [M+H]⁺.

### Step-6: Synthesis of N-{2-[4-(3-amino-2-fluorophenyl)-5-(2-chloropyrimidin-4-yl)-1,3-thiazol-2-yl]-2-methylpropyl}oxane-4-carboxamide (7)

To a mixture of prop-2-en-1-yl N-{3-[5-(2-chloropyrimidin-4-yl)-2-[2-methyl-1-(oxan-4-ylformamido)propan-2-yl]-1,3-thiazol-4-yl]-2-fluorophenyl}carbamate (140 mg, 0.244 mmol, 1 equiv) and Pd(PPh₃)₂Cl₂ (3.42 mg, 0.005 mmol, 0.02 equiv) in DCM (2 mL) was added HOAc (35.15 mg, 0.586 mmol, 2.4 equiv) and n-Bu₃SnH (106.47 mg, 0.366 mmol, 1.5 equiv) at 0° C. The resulting mixture was stirred for 0.5 h at 0° C. The reaction was quenched by the addition of saturated NaHCO₃ aqueous at 0° C. The resulting mixture was extracted with CH₂Cl₂. The combined organic layers were washed with water and dried over anhydrous Na₂SO₄. After filtration, the filtrate was concentrated under reduced pressure to afford N-{2-[4-(3-amino-2-fluorophenyl)-5-(2-chloropyrimidin-4-yl)-1,3-thiazol-2-yl]-2-methylpropyl}oxane-4-carboxamide (120 mg, crude) as a yellow oil. LCMS: C₂₃H₂₅ClFN₅O₂S requires: 489.1, found: m/z=490.1 [M+H]⁺.

### Step-7: Synthesis of N-(2-(5-(2-chloropyrimidin-4-yl)-4-(2-fluoro-3-(propylsulfonamido)phenyl)thiazol-2-yl)-2-methylpropyl)tetrahydro-2H-pyran-4-carboxamide (8)

To a mixture of N-{2-[4-(3-amino-2-fluorophenyl)-5-(2-chloropyrimidin-4-yl)-1,3-thiazol-2-yl]-2-methylpropyl}oxane-4-carboxamide (80 mg, 0.163 mmol, 1 equiv) in DCM (2 mL) was added TEA (49.56 mg, 0.489 mmol, 3 equiv) and propane-1-sulfonyl chloride (34.92 mg, 0.244 mmol, 1.5 equiv) at room temperature. The resulting mixture was stirred for 2 h at room temperature. The resulting mixture was concentrated under reduced pressure to afford N-(2-(5-(2-chloropyrimidin-4-yl)-4-(2-fluoro-3-(propylsulfonamido)phenyl)thiazol-2-yl)-2-methylpropyl)tetrahydro-2H-pyran-4-carboxamide (100 mg, crude) as a brown oil. LCMS: C₂₆H₃₁ClFN₅O₄S₂ requires: 595.1, found: m/z=596.1 [M+H]⁺.

### Step-8: Synthesis of tert-butyl N-(4-{4-[2-fluoro-3-(propane-1-sulfonamido)phenyl]-2-[2-methyl-1-(oxan-4-ylformamido)propan-2-yl]-1,3-thiazol-5-yl}pyrimidin-2-yl)carbamate (9)

To a mixture of N-{2-[5-(2-chloropyrimidin-4-yl)-4-[2-fluoro-3-(propane-1-sulfonamido)phenyl]-1,3-thiazol-2-yl]-2-methylpropyl}oxane-4-carboxamide (70 mg, 0.117 mmol, 1 equiv) and tert-butyl carbamate (20.63 mg, 0.176 mmol, 1.5 equiv) in dioxane (2 mL) was added Pd(OAc)₂ (2.64 mg, 0.012 mmol, 0.1 equiv), Cs₂CO₃ (61.21 mg, 0.187 mmol, 1.6 equiv), and BINAP (21.94 mg, 0.035 mmol, 0.3 equiv) at room temperature under a nitrogen atmosphere. The resulting mixture was stirred for 3 h at 90° C. under the nitrogen atmosphere. The resulting mixture was concentrated under reduced pressure. The residue was purified by Prep-TLC CH₂Cl₂:EtOAc (1:1) to afford tert-butyl N-(4-{4-[2-fluoro-3-(propane-1-sulfonamido)phenyl]-2-[2-methyl-1-(oxan-4-ylformamido)propan-2-yl]-1,3-thiazol-5-yl}pyrimidin-2-yl)carbamate (45 mg, 50.96%) as a white solid. LCMS: C₃₁H₄₁FN₆O₆S₂ requires: 676.3, found: m/z=677.2 [M+H]⁺.

### Step-9: Synthesis of N-{2-[5-(2-aminopyrimidin-4-yl)-4-[2-fluoro-3-(propane-1-sulfonamido)phenyl]-1,3-thiazol-2-yl]-2-methylpropyl}oxane-4-carboxamide

A solution of tert-butyl N-(4-{4-[2-fluoro-3-(propane-1-sulfonamido)phenyl]-2-[2-methyl-1-(oxan-4-ylformamido)propan-2-yl]-1,3-thiazol-5-yl}pyrimidin-2-yl)carbamate (35 mg, 0.052 mmol, 1 equiv) in HCl/1,4-dioxane (5 mL, 4 M) was stirred overnight at room temperature. The resulting mixture was concentrated under reduced pressure. The crude product was purified by reverse phase flash chromatography with H₂O:MeCN (3:1) to afford N-{2-[5-(2-aminopyrimidin-4-yl)-4-[2-fluoro-3-(propane-1-sulfonamido)phenyl]-1,3-thiazol-2-yl]-2-methylpropyl}oxane-4-carboxamide (28.8 mg, 92.71%) as an off-white solid. LCMS: C₂₆H₃₃FN₆O₄S₂ requires: 576.2, found: m/z=577.2 [M+H]⁺.

¹H NMR (400 MHz, CD₃OD) δ 8.10-8.08 (m, 1H), 7.71-7.67 (m, 1H), 7.53-7.51 (m, 1H), 7.40-7.36 (m, 1H), 6.67-6.64 (m, 1H), 3.92-3.88 (m, 2H), 3.56-3.54 (m, 2H), 3.42-3.33 (m, 2H), 3.18-3.14 (m, 2H), 2.57-2.42 (m, 1H), 1.90-1.84 (m, 2H), 1.74-1.62 (m, 4H), 1.52 (s, 6H), 1.06-1.04 (m, 3H).

### Synthesis of N-(3-(5-(2-aminopyrimidin-4-yl)-2-(2-(piperidin-4-yl)propan-2-yl)thiazol-4-yl)-2-fluorophenyl)propane-1-sulfonamide

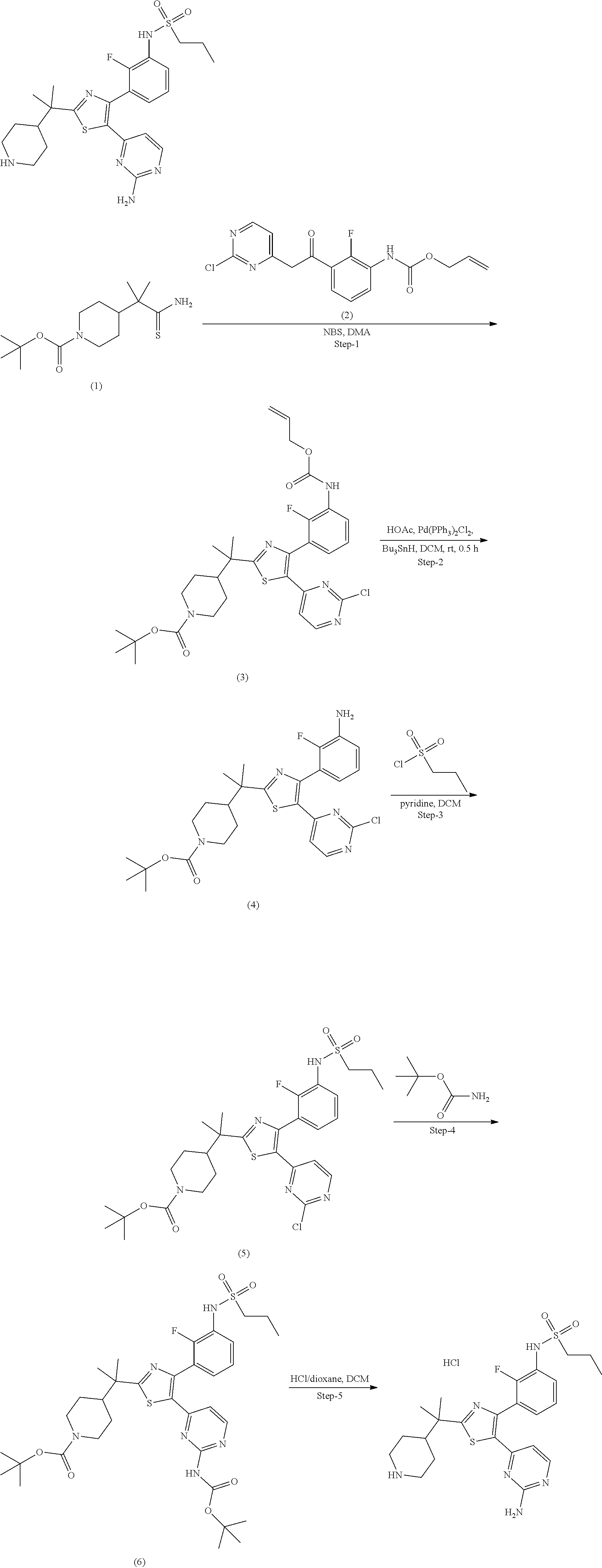

### Step-1: Synthesis of tert-butyl 4-(2-(4-(3-(((allyloxy)carbonyl)amino)-2-fluorophenyl)-5-(2-chloropyrimidin-4-yl)thiazol-2-yl)propan-2-yl)piperidine-1-carboxylate (3)

To a mixture of prop-2-en-1-yl N-{3-[2-(2-chloropyrimidin-4-yl)acetyl]-2-fluorophenyl}carbamate (2.4 g, 6.98 mmol, 1 equiv) in DMA (20.0 mL) was added NBS (1.24 g, 6.98 mmol, 1 equiv) for 15 min at room temperature followed by the addition of tert-butyl 4-(1-carbamothioyl-1-methylethyl)piperidine-1-carboxylate (2.0 g, 6.98 mmol, 1 equiv) at room temperature. The resulting mixture was stirred for 2 h at 60° C. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with PE:EtOAc (3:1) to afford tert-butyl 4-{2-[5-(2-chloropyrimidin-4-yl)-4-(2-fluoro-3-{[(prop-2-en-1-yloxy)carbonyl]amino}phenyl)-1,3-thiazol-2-yl]propan-2-yl}piperidine-1-carboxylate (1.80 g, 41.84%) as a white solid. LCMS: C₃₀H₃₅ClFN₅O₄S requires: 615.2, found: m/z=616.2 [M+H]⁺.

### Step-2: Synthesis of tert-butyl 4-{2-[4-(3-amino-2-fluorophenyl)-5-(2-chloropyrimidin-4-yl)-1,3-thiazol-2-yl]propan-2-yl}piperidine-1-carboxylate (4)

To a mixture of tert-butyl 4-{2-[5-(2-chloropyrimidin-4-yl)-4-(2-fluoro-3-{[(prop-2-en-1-yloxy)carbonyl]amino}phenyl)-1,3-thiazol-2-yl]propan-2-yl}piperidine-1-carboxylate (1.0 g, 1.623 mmol, 1 equiv) and HOAc (243.66 mg, 4.058 mmol, 2.5 equiv) in DCM (4 mL) was added Pd(PPh₃)₂Cl₂ (22.78 mg, 0.032 mmol, 0.02 equiv) and n-Bu₃SnH (755.83 mg, 2.597 mmol, 1.6 equiv) at room temperature under a nitrogen atmosphere. The resulting mixture was stirred for 0.5 h at room temperature under the nitrogen atmosphere. The resulting mixture was extracted with EtOAc. The combined organic layers were washed with water and dried over anhydrous Na₂SO₄. After filtration, the filtrate was concentrated under reduced pressure to afford tert-butyl 4-{2-[4-(3-amino-2-fluorophenyl)-5-(2-chloropyrimidin-4-yl)-1,3-thiazol-2-yl]propan-2-yl}piperidine-1-carboxylate (700 mg, crude) as a brown oil. The crude product was used in the next step directly without further purification. LCMS: C₂₆H₃₁ClFN₅O₂S requires: 531.2, found: m/z=532.2 [M+H]⁺.

### Step-3: Synthesis of tert-butyl 4-{2-[5-(2-chloropyrimidin-4-yl)-4-[2-fluoro-3-(propane-1-sulfonamido)phenyl]-1,3-thiazol-2-yl]propan-2-yl}piperidine-1-carboxylate (5)

To a mixture of tert-butyl 4-{2-[4-(3-amino-2-fluorophenyl)-5-(2-chloropyrimidin-4-yl)-1,3-thiazol-2-yl]propan-2-yl}piperidine-1-carboxylate (1.6 g, 3.007 mmol, 1 equiv) and pyridine (0.24 g, 3.007 mmol, 1.0 equiv) in DCM (15 mL) was added propane-1-sulfonyl chloride (0.43 g, 3.007 mmol, 1.0 equiv) dropwise at 0° C. The resulting mixture was stirred for 2 h at room temperature. The resulting mixture was concentrated under vacuum. The crude product was dissolved in THF (5 mL), ACN (5 mL), and H₂O (5 mL). Na₂CO₃ (3.7 g, 35.190 mmol, 6 equiv) was added at room temperature. The resulting mixture was stirred at 50° C. overnight. The resulting mixture was concentrated under vacuum. The resulting mixture was extracted with DCM. The combined organic layers were washed with water and dried over anhydrous Na₂SO₄. After filtration, the filtrate was concentrated under reduced pressure. The residue was purified by silica gel column chromatography eluted with PE:EtOAc (5:1) to afford tert-butyl 4-{2-[5-(2-chloropyrimidin-4-yl)-4-[2-fluoro-3-(propane-1-sulfonamido)phenyl]-1,3-thiazol-2-yl]propan-2-yl}piperidine-1-carboxylate (1.0 g, 52.11%) as a yellow solid. LCMS: C₂₉H₃₇ClFN₅O₄S₂ requires: 637.2, found: m/z=638.2 [M+H]⁺.

### Step-4: Synthesis of tert-butyl 4-[2-(5-{2-[(tert-butoxycarbonyl)amino]pyrimidin-4-yl}-4-[2-fluoro-3-(propane-1-sulfonamido)phenyl]-1,3-thiazol-2-yl)propan-2-yl]piperidine-1-carboxylate (6)

To a mixture of tert-butyl 4-{2-[5-(2-chloropyrimidin-4-yl)-4-[2-fluoro-3-(propane-1-sulfonamido)phenyl]-1,3-thiazol-2-yl]propan-2-yl}piperidine-1-carboxylate (1.0 g, 1.567 mmol, 1 equiv) and BocNH₂ (917.79 mg, 7.835 mmol, 5.0 equiv) in 1,4-dioxane (10 mL) was added BrettPhos Pd G₃ (142.0 mg, 0.157 mmol, 0.1 equiv) and Cs₂CO₃ (1.02 g, 3.134 mmol, 2.0 equiv) at room temperature under a nitrogen atmosphere. The resulting mixture was stirred at 80° C. overnight under the nitrogen atmosphere. The resulting mixture was concentrated under reduced pressure. The residue was purified by silica gel column chromatography eluted with PE:EtOAc (1:1) to afford tert-butyl 4-[2-(5-{2-[(tert-butoxycarbonyl)amino]pyrimidin-4-yl}-4-[2-fluoro-3-(propane-1-sulfonamido)phenyl]-1,3-thiazol-2-yl)propan-2-yl]piperidine-1-carboxylate (800 mg, 82.51%) as a yellow solid. LCMS: C₃₄H₄₇FN₆O₆S₂ requires: 718.3, found: m/z=719.1 [M+H]⁺.

### Step-5: Synthesis of N-{3-[5-(2-aminopyrimidin-4-yl)-2-[2-(piperidin-4-yl)propan-2-yl]-1,3-thiazol-4-yl]-2-fluorophenyl}propane-1-sulfonamide Hydrochloride

To a mixture of tert-butyl 4-[2-(5-{2-[(tert-butoxycarbonyl)amino]pyrimidin-4-yl}-4-[2-fluoro-3-(propane-1-sulfonamido)phenyl]-1,3-thiazol-2-yl)propan-2-yl]piperidine-1-carboxylate (800 mg, 1.113 mmol, 1 equiv) in DCM (8 mL) was added HCl (gas) in 1,4-dioxane (2 mL, 4 M). The resulting mixture was stirred at room temperature for 2 h. The resulting mixture was concentrated under reduced pressure. The residue was purified by reverse phase flash chromatography with the following conditions: column, C18 silica gel; mobile phase, MeCN in Water (0.1% formic acid (FA)), 10% to 50% gradient in 10 min; detector, UV 254 nm to afford N-{3-[5-(2-aminopyrimidin-4-yl)-2-[2-(piperidin-4-yl)propan-2-yl]-1,3-thiazol-4-yl]-2-fluorophenyl}propane-1-sulfonamide hydrochloride (553 mg, 89.52%) as a yellow solid. LCMS: C₂₄H₃₁FN₆O₂S₂ requires: 518.2, found: m/z=519.2 [M+H]⁺.

¹H NMR (400 MHz, CD₃OD) δ 8.10 (d, J=6.8 Hz, 1H), 7.69 (d, J=1.6 Hz, 1H), 7.52-7.49 (m, 1H), 7.40-7.36 (m, 1H), 6.66-6.64 (m, 1H), 3.51-3.40 (m, 2H), 3.23-3.13 (m, 2H), 3.05-2.85 (m, 2H), 2.24-2.16 (m, 1H), 2.01-1.82 (m, 4H), 1.71-1.65 (m, 2H), 1.55 (s, 6H), 1.10-1.07 (m, 3H).

### Synthesis of N-(3-(5-(2-aminopyrimidin-4-yl)-2-(piperidin-4-yl)thiazol-4-yl)-2-fluorophenyl)propane-1-sulfonamide Hydrochloride Salt

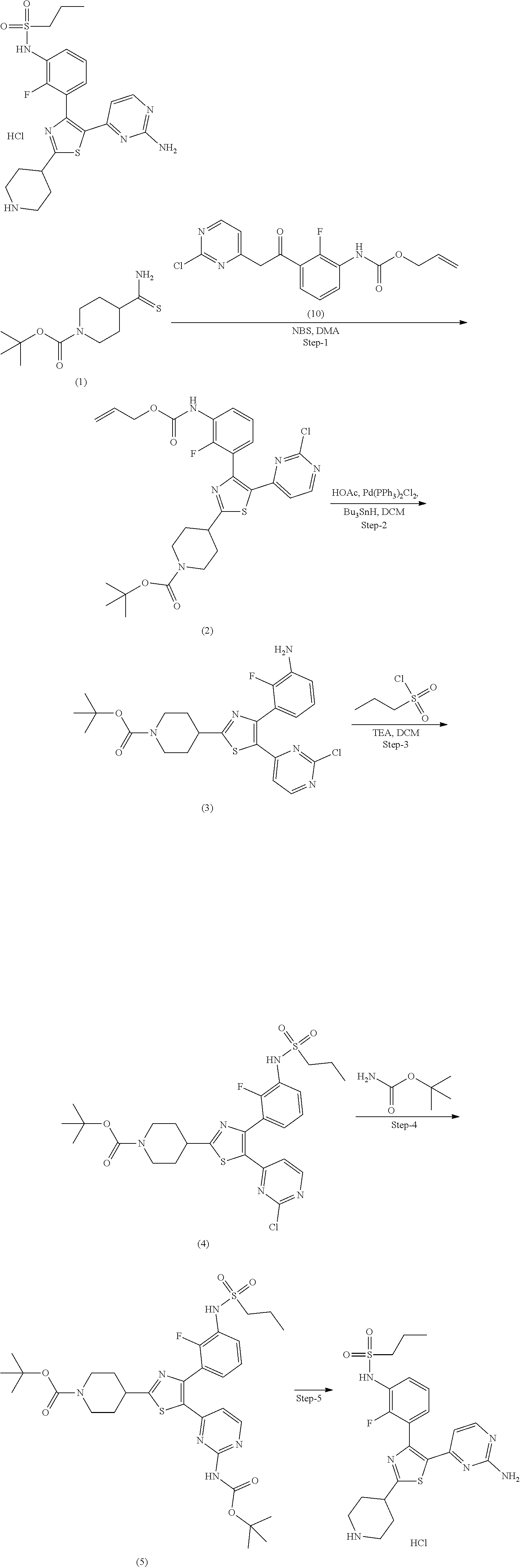

### Step-1: Synthesis of tert-butyl 4-[5-(2-chloropyrimidin-4-yl)-4-(2-fluoro-3-{[(prop-2-en-1-yloxy)carbonyl]amino}phenyl)-1,3-thiazol-2-yl]piperidine-1-carboxylate (2)

To a mixture of prop-2-en-1-yl N-{3-[2-(2-chloropyrimidin-4-yl)acetyl]-2-fluorophenyl}carbamate (1.1 g, 3.145 mmol, 1.00 equiv) and NBS (559.8 mg, 3.145 mmol, 1.0 equiv) in DMA (13 mL) was added tert-butyl 4-carbamothioylpiperidine-1-carboxylate (922.2 mg, 3.774 mmol, 1.20 equiv) at room temperature. The resulting mixture was stirred for 2 h at 60° C. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with PE:EtOAc (1:1) to afford tert-butyl 4-[5-(2-chloropyrimidin-4-yl)-4-(2-fluoro-3-{[(prop-2-en-1-yloxy)carbonyl]amino}phenyl)-1,3-thiazol-2-yl]piperidine-1-carboxylate (580 mg, 32.12%) as a yellow solid. LCMS: C₂₇H₂₉ClFN₅O₄S requires: 573.2, found: m/z=574.2 [M+H]⁺.

### Step-2: Synthesis of tert-butyl 4-[4-(3-amino-2-fluorophenyl)-5-(2-chloropyrimidin-4-yl)-1,3-thiazol-2-yl]piperidine-1-carboxylate (3)

To a mixture of tert-butyl 4-[5-(2-chloropyrimidin-4-yl)-4-(2-fluoro-3-{[(prop-2-en-1-yloxy)carbonyl]amino}phenyl)-1,3-thiazol-2-yl]piperidine-1-carboxylate (600 mg, 1.045 mmol, 1 equiv) and HOAc (156.9 mg, 2.612 mmol, 2.5 equiv) in DCM (8 mL) was added Pd(PPh₃)₂Cl₂ (14.7 mg, 0.021 mmol, 0.02 equiv) and n-Bu₃SnH (486.7 mg, 1.672 mmol, 1.6 equiv) in portions at 0° C. The resulting mixture was stirred for 0.5 h at room temperature under a nitrogen atmosphere. The resulting mixture was concentrated under vacuum to afford tert-butyl 4-[4-(3-amino-2-fluorophenyl)-5-(2-chloropyrimidin-4-yl)-1,3-thiazol-2-yl]piperidine-1-carboxylate (510 mg, crude) as a red oil. The resulting mixture was used in the next step directly without further purification. LCMS: C₂₃H₂₅ClFN₅O₂S requires: 489.1, found: m/z=490.1 [M+H]⁺.

### Step-3: Synthesis of tert-butyl 4-(5-(2-chloropyrimidin-4-yl)-4-(2-fluoro-3-(propylsulfonamido)phenyl)thiazol-2-yl)piperidine-1-carboxylate (4)

To a mixture of tert-butyl 4-[4-(3-amino-2-fluorophenyl)-5-(2-chloropyrimidin-4-yl)-1,3-thiazol-2-yl]piperidine-1-carboxylate (2 g, 4.082 mmol, 1 equiv) and TEA (1.24 g, 12.246 mmol, 3 equiv) in DCM (20 mL) was added propane-1-sulfonyl chloride (873.08 mg, 6.123 mmol, 1.5 equiv) dropwise at 0° C. The resulting mixture was stirred for 2 h at room temperature. The resulting mixture was concentrated under vacuum. To the crude product in H₂O (7 mL), MeCN (7 mL), and THF (7 mL) was added Na₂CO₃ (1.06 g, 10.065 mmol, 2.46 equiv) at room temperature. The resulting mixture was stirred at 50° C. overnight. The resulting mixture was extracted with DCM. The combined organic layers were washed with water and dried over anhydrous Na₂SO₄. After filtration, the filtrate was concentrated under reduced pressure. The residue was purified by silica gel column chromatography eluted with CH₂Cl₂:MeOH (10:1) to afford tert-butyl 4-(5-(2-chloropyrimidin-4-yl)-4-(2-fluoro-3-(propylsulfonamido)phenyl)thiazol-2-yl)piperidine-1-carboxylate (1.58 g, 64.93%) as a yellow solid. LCMS: C₂₆H₃₁ClFN₅O₄S₂ requires: 595.1, found: m/z=596.1 [M+H]⁺.

### Step-4: Synthesis of tert-butyl 4-(5-(2-((tert-butoxycarbonyl)amino)pyrimidin-4-yl)-4-(2-fluoro-3-(propylsulfonamido)phenyl)thiazol-2-yl)piperidine-1-carboxylate (5)

To a mixture of tert-butyl 4-(5-(2-chloropyrimidin-4-yl)-4-(2-fluoro-3-(propylsulfonamido)phenyl)thiazol-2-yl)piperidine-1-carboxylate (1 g, 1.677 mmol, 1 equiv) and tert-butyl carbamate (982.6 mg, 8.385 mmol, 5 equiv) in 1,4-dioxane (10 mL) was added BrettPhos Pd G₃ (152 mg, 0.168 mmol, 0.1 equiv) and Cs₂CO₃ (1.1 g, 3.354 mmol, 2 equiv) at room temperature. The resulting mixture was stirred for 2 h at 80° C. under a nitrogen atmosphere. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with PE:EtOAc (1:1) to afford tert-butyl 4-(5-(2-((tert-butoxycarbonyl)amino)pyrimidin-4-yl)-4-(2-fluoro-3-(propylsulfonamido)phenyl)thiazol-2-yl)piperidine-1-carboxylate (850 mg, 74.87%) as a yellow solid. LCMS: C₃₁H₄₁FN₆O₆S₂ requires: 676.3, found: m/z=677.3 [M+H]⁺.

### Step-5: Synthesis of N-{3-[5-(2-aminopyrimidin-4-yl)-2-(piperidin-4-yl)-1,3-thiazol-4-yl]-2-fluorophenyl}propane-1-sulfonamide Hydrochloride

To a mixture of tert-butyl 4-(5-(2-((tert-butoxycarbonyl)amino)pyrimidin-4-yl)-4-(2-fluoro-3-(propylsulfonamido)phenyl)thiazol-2-yl)piperidine-1-carboxylate (1.5 g, 2.217 mmol, 1 equiv) in DCM (3 mL) was added HCl (gas) in 1,4-dioxane (12 mL, 4 M) at room temperature. The resulting mixture was stirred at room temperature overnight. The resulting mixture was concentrated under reduced pressure. The residue was purified by reverse phase flash chromatography with the following conditions: column, C18 silica gel; mobile phase, MeCN in water (0.1% formic acid (FA)), 10% to 50% gradient in 10 min; detector, UV 254 nm to afford N-{3-[5-(2-aminopyrimidin-4-yl)-2-(piperidin-4-yl)-1,3-thiazol-4-yl]-2-fluorophenyl}propane-1-sulfonamide hydrochloride salt (649.6 mg, 57.13%) as a yellow solid. LCMS: C₂₁H₂₅FN₆O₂S₂ requires: 476.3, found: m/z=477.3 [M+H]⁺. ¹H NMR (400 MHz, CD₃OD) δ 8.09 (d, J=6.0 Hz, 1H), 7.68-7.66 (m, 1H), 7.49-7.28 (m, 2H), 6.65-6.63 (m, 1H), 3.60-3.48 (m, 3H), 3.28-3.16 (m, 2H), 3.17-3.05 (m, 2H), 2.49-2.39 (m, 2H), 2.21-2.09 (m, 2H), 1.94-1.78 (m, 2H), 1.14-0.98 (m, 3H).

### Synthesis of N-{2-fluoro-3-[2-(piperidin-4-yl)-5-(pyrimidin-4-yl)-1,3-thiazol-4-yl]phenyl}propane-1-sulfonamide Hydrochloride Salt

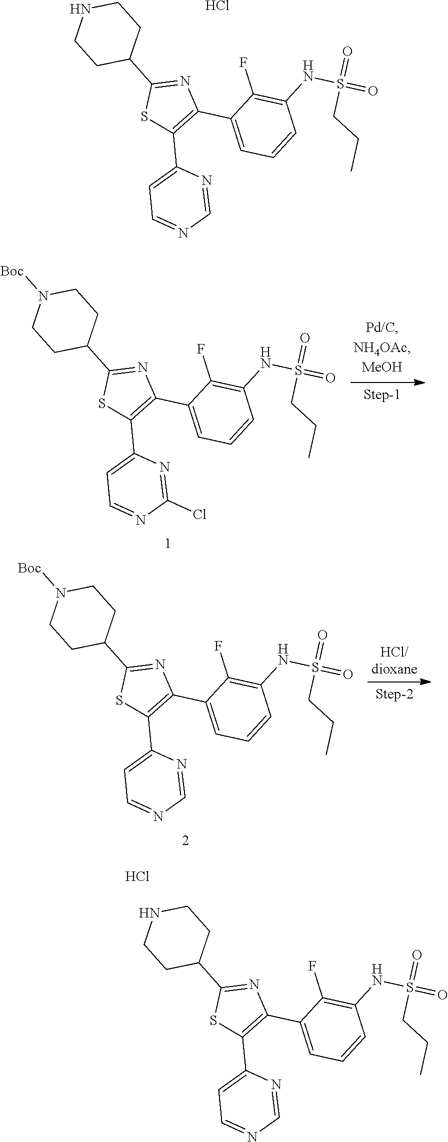 [SMILES: CCCS(=O)(=O)Nc1cccc(-c2nc(C3CCNCC3)sc2-c2ccncn2)c1F ; Cl ; CCCS(=O)(=O)Nc1cccc(-c2nc(C3CCN(C(=O)OC(C)(C)C)CC3)sc2-c2ccnc(Cl)n2)c1F ; CCCS(=O)(=O)Nc1cccc(-c2nc(C3CCN(C(=O)OC(C)(C)C)CC3)sc2-c2ccncn2)c1F ; Cl ; CCCS(=O)(=O)Nc1cccc(-c2nc(C3CCNCC3)sc2-c2ccncn2)c1F]

### Step-1: Synthesis of tert-butyl 4-{4-[2-fluoro-3-(propane-1-sulfonamido)phenyl]-5-(pyrimidin-4-yl)-1,3-thiazol-2-yl}piperidine-1-carboxylate (2)

To a mixture of tert-butyl 4-[5-(2-chloropyrimidin-4-yl)-4-[2-fluoro-3-(propane-1-sulfonamido)phenyl]-1,3-thiazol-2-yl]piperidine-1-carboxylate (700 mg, 1.174 mmol, 1 equiv) and NH₄OAc (905.14 mg, 11.740 mmol, 10 equiv) in MeOH (10 mL) was added Pd/C (700 mg) at room temperature. The resulting mixture was stirred for 30 min at 40° C. The resulting mixture was filtered. The filtrate was concentrated under reduced pressure. The residue was purified by C18 silica gel column chromatography eluted with CH₃CN:H₂O (1:1) to afford tert-butyl 4-{4-[2-fluoro-3-(propane-1-sulfonamido)phenyl]-5-(pyrimidin-4-yl)-1,3-thiazol-2-yl}piperidine-1-carboxylate (450 mg, 68.23%) as a yellow solid. LCMS: C₂₆H₃₂FN₅O₄S₂ requires: 561.2, found: m/z=562.2 [M+H]⁺.

### Step-2: Synthesis of N-{2-fluoro-3-[2-(piperidin-4-yl)-5-(pyrimidin-4-yl)-1,3-thiazol-4-yl]phenyl}propane-1-sulfonamide Hydrochloride

A mixture of tert-butyl 4-{4-[2-fluoro-3-(propane-1-sulfonamido)phenyl]-5-(pyrimidin-4-yl)-1,3-thiazol-2-yl}piperidine-1-carboxylate (450 mg, 0.801 mmol, 1 equiv) in HCl/1,4-dioxane (5 mL, 4N) was stirred overnight at room temperature. The resulting mixture was concentrated under reduced pressure. This resulted in N-{2-fluoro-3-[2-(piperidin-4-yl)-5-(pyrimidin-4-yl)-1,3-thiazol-4-yl]phenyl}propane-1-sulfonamide hydrochloride salt (300.1 mg, crude) as a yellow solid. LCMS: C₂₁H₂₄FN₅O₂S₂ requires: 461.1, found: m/z=462.1 [M+H]⁺. ¹H NMR (400 MHz, CD₃OD) δ 9.30-9.28 (m, 1H), 8.76-8.74 (m, 1H), 7.71-7.67 (m, 1H), 7.50-7.38 (m, 3H), 3.60-3.55 (m, 3H), 3.27-3.22 (m, 2H), 3.16-3.12 (m, 2H), 2.48-2.43 (m, 2H), 2.20-2.14 (m, 2H), 1.89-1.83 (m, 2H), 1.06-1.03 (m, 3H).

### Synthesis of N-{3-[5-(2-aminopyrimidin-4-yl)-2-[4-(piperidin-4-yl)phenyl]-1,3-thiazol-4-yl]-2-fluorophenyl}propane-1-sulfonamide

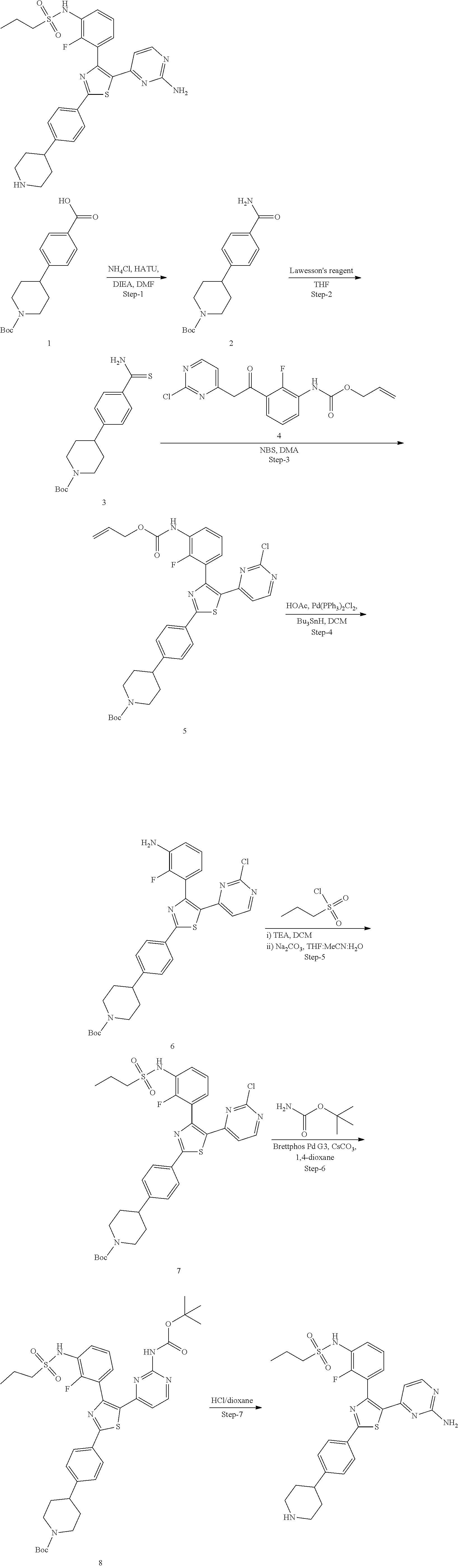

### Step-1: Synthesis of tert-butyl 4-(4-carbamoylphenyl)piperidine-1-carboxylate (2)

To a mixture of 4-[1-(tert-butoxycarbonyl)piperidin-4-yl]benzoic acid (5 g, 16.37 mmol, 1 equiv) and NH₄Cl (4.37 g, 81.86 mmol, 5 equiv) in DMF (50 mL) was added HATU (9.33 g, 24.55 mmol, 1.5 equiv) and DIEA (6.34 g, 49.11 mmol, 3 equiv) at room temperature. The resulting mixture was stirred for one hour at room temperature. The resulting mixture was extracted with EtOAc. The combined organic layers were washed with water and dried over anhydrous Na₂SO₄. After filtration, the filtrate was concentrated under reduced pressure. The product was washed with EtOAc. The precipitated solids were collected by filtration to afford tert-butyl 4-(4-carbamoylphenyl)piperidine-1-carboxylate (4.8 g, 96.31%) as a white solid. LCMS: C₁₇H₂₄N₂O₃ requires: 304.2, found: m/z=305.2 [M+H]⁺.

### Step-2: Synthesis of tert-butyl 4-(4-carbamothioylphenyl)piperidine-1-carboxylate (3)

To a mixture of tert-butyl 4-(4-carbamoylphenyl)piperidine-1-carboxylate (3 g, 9.85 mmol, 1 equiv) in THF (30 mL) was added Lawesson's Reagent (1.99 g, 4.92 mmol, 0.5 equiv) at room temperature. The resulting mixture was stirred overnight at 50° C. The resulting mixture was extracted with EtOAc. The combined organic layers were washed with aqueous NaHCO₃ and dried over anhydrous Na₂SO₄. After filtration, the filtrate was concentrated under reduced pressure. The residue was purified by silica gel column chromatography eluted with CH₂Cl₂:EtOAc (4:1) to afford tert-butyl 4-(4-carbamothioylphenyl)piperidine-1-carboxylate (2.2 g, 69.66%) as a white solid. LCMS: C₁₇H₂₄N₂O₂S requires: 320.1, found: m/z=321.2 [M+H]⁺.

### Step-3: Synthesis of tert-butyl 4-{4-[5-(2-chloropyrimidin-4-yl)-4-(2-fluoro-3-{[(prop-2-en-1-yloxy)carbonyl]amino}phenyl)-1,3-thiazol-2-yl]phenyl}piperidine-1-carboxylate (5)

To a mixture of prop-2-en-1-yl N-{3-[2-(2-chloropyrimidin-4-yl)acetyl]-2-fluorophenyl}carbamate (1.8 g, 5.14 mmol, 1 equiv) in DMA (20 mL) was added tert-butyl 4-(4-carbamothioylphenyl)piperidine-1-carboxylate (1.98 g, 6.17 mmol, 1.2 equiv) and NBS (1.83 g, 10.29 mmol, 2 equiv) at room temperature. The resulting mixture was stirred for 2 h at 60° C. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with PE:EtOAc (5:1) to afford tert-butyl 4-{4-[5-(2-chloropyrimidin-4-yl)-4-(2-fluoro-3-{[(prop-2-en-1-yloxy)carbonyl]amino}phenyl)-1,3-thiazol-2-yl]phenyl}piperidine-1-carboxylate (1.8 g, 48.95%) as a yellow solid. LCMS: C₃₃H₃₃ClFN₅O₄S requires: 649.2, found: m/z=650.2 [M+H]⁺.

### Step-4: Synthesis of tert-butyl 4-{4-[4-(3-amino-2-fluorophenyl)-5-(2-chloropyrimidin-4-yl)-1,3-thiazol-2-yl]phenyl}piperidine-1-carboxylate (6)

To a mixture of tert-butyl 4-{4-[5-(2-chloropyrimidin-4-yl)-4-(2-fluoro-3-{[(prop-2-en-1-yloxy)carbonyl]amino}phenyl)-1,3-thiazol-2-yl]phenyl}piperidine-1-carboxylate (1.8 g, 2.76 mmol, 1 equiv) and HOAc (415 mg, 6.93 mmol, 2.5 equiv) in DCM (20 mL) was added n-Bu₃SnH (1.29 g, 4.43 mmol, 1.6 equiv) and Pd(PPh₃)₂Cl₂ (39 mg, 0.05 mmol, 0.02 equiv) at 0° C. under a nitrogen atmosphere. The resulting mixture was stirred for 30 min at room temperature. The reaction was quenched with saturated aqueous NaHCO₃ at 0° C. The resulting mixture was extracted with CH₂Cl₂. The combined organic layers were washed with water and dried over anhydrous Na₂SO₄. After filtration, the filtrate was concentrated under reduced pressure to afford tert-butyl 4-{4-[4-(3-amino-2-fluorophenyl)-5-(2-chloropyrimidin-4-yl)-1,3-thiazol-2-yl]phenyl}piperidine-1-carboxylate (1.5 g, crude) as a brown solid. LCMS: C₂₉H₂₉ClFN₅O₂S requires: 565.2, found: m/z=566.2 [M+H]⁺.

### Step-5: Synthesis of tert-butyl 4-(4-(5-(2-chloropyrimidin-4-yl)-4-(2-fluoro-3-(propylsulfonamido)phenyl)thiazol-2-yl)phenyl)piperidine-1-carboxylate (7)

To a mixture of tert-butyl 4-{4-[4-(3-amino-2-fluorophenyl)-5-(2-chloropyrimidin-4-yl)-1,3-thiazol-2-yl]phenyl}piperidine-1-carboxylate (1.5 g, 2.65 mmol, 1 equiv) in DCM (15 mL) was added propane-1-sulfonyl chloride (755 mg, 5.30 mmol, 2 equiv) and TEA (804 mg, 7.95 mmol, 3 equiv) at 0° C. The resulting mixture was stirred for one hour at room temperature. The resulting mixture was concentrated under reduced pressure. The crude product was dissolved in THF (5 mL), H₂O (5 mL), and MeCN (5 mL). Na₂CO₃ (1.13 g, 10.68 mmol, 5 equiv) was added at room temperature. The resulting mixture was stirred overnight at 50° C. The resulting mixture was concentrated under vacuum. The residue was extracted with DCM. The combined organic layers were washed with water and dried over anhydrous Na₂SO₄. After filtration, the filtrate was concentrated under reduced pressure. The residue was purified by silica gel column chromatography eluted with DCM:MeOH (10:1) to afford tert-butyl 4-(4-(5-(2-chloropyrimidin-4-yl)-4-(2-fluoro-3-(propylsulfonamido)phenyl)thiazol-2-yl)phenyl)piperidine-1-carboxylate (1.1 g, 73.77%) as a yellow solid. LCMS: C₃₂H₃₅ClFN₅O₄S₂ requires: 671.2, found: m/z=672.2 [M+H]⁺.

### Step-6: Synthesis of tert-butyl 4-[4-(5-{2-[(tert-butoxycarbonyl)amino]pyrimidin-4-yl}-4-[2-fluoro-3-(propane-1-sulfonamido)phenyl]-1,3-thiazol-2-yl)phenyl]piperidine-1-carboxylate (8)

To a mixture of tert-butyl 4-{4-[5-(2-chloropyrimidin-4-yl)-4-[2-fluoro-3-(propane-1-sulfonamido)phenyl]-1,3-thiazol-2-yl]phenyl}piperidine-1-carboxylate (600 mg, 0.89 mmol, 1 equiv) and tert-butyl carbamate (522.80 mg, 4.465 mmol, 5 equiv) in dioxane (6 mL) was added BrettPhos Pd G3 (81 mg, 0.089 mmol, 0.1 equiv) and Cs₂CO₃ (581 mg, 1.78 mmol, 2 equiv) at room temperature under a nitrogen atmosphere. The resulting mixture was stirred for 2 h at 80° C. The resulting mixture was concentrated under reduced pressure. The residue was purified by silica gel column chromatography eluted with PE:EtOAc (1:1) to afford tert-butyl 4-[4-(5-{2-[(tert-butoxycarbonyl)amino]pyrimidin-4-yl}-4-[2-fluoro-3-(propane-1-sulfonamido)phenyl]-1,3-thiazol-2-yl)phenyl]piperidine-1-carboxylate (400 mg, 53.57%) as a yellow solid. LCMS: C₃₇H₄₅FN₆O₆S₂ requires: 752.2, found: m/z=753.3 [M+H]⁺.

### Step-7: Synthesis of N-{3-[5-(2-aminopyrimidin-4-yl)-2-[4-(piperidin-4-yl)phenyl]-1,3-thiazol-4-yl]-2-fluorophenyl}propane-1-sulfonamide

A mixture of tert-butyl 4-[4-(5-{2-[(tert-butoxycarbonyl)amino]pyrimidin-4-yl}-4-[2-fluoro-3-(propane-1-sulfonamido)phenyl]-1,3-thiazol-2-yl)phenyl]piperidine-1-carboxylate (400 mg, 0.53 mmol, 1 equiv) in HCl/1,4-dioxane (5 mL, 4 M) was stirred overnight at room temperature. The resulting mixture was concentrated under reduced pressure. The crude product was purified by Prep-HPLC with the following conditions: Column: XBridge Prep OBD C18 Column, 30*150 mm, 5 m; Mobile Phase A: water (10 mmol/L NH₄HCO₃), Mobile Phase B: ACN; Flow rate: 60 mL/min; Gradient: 30% B to 38% B in 8 min; Wave Length: 220/254 nm; Rₜ₁ (min): 6.75 to afford N-{3-[5-(2-aminopyrimidin-4-yl)-2-[4-(piperidin-4-yl)phenyl]-1,3-thiazol-4-yl]-2-fluorophenyl}propane-1-sulfonamide (223.7 mg, 70.01%) as a yellow solid. LCMS: C₂₇H₂₉FN₆O₂S₂ requires: 552.2, found: m/z=553.2 [M+H]⁺. ¹H NMR (400 MHz, DMSO-d₆) δ 8.15-8.06 (m, 1H), 7.98-7.93 (m, 2H), 7.56-7.52 (m, 1H), 7.40-7.37 (m, 2H), 7.24-7.16 (m, 2H), 6.79 (s, 2H), 6.18-6.16 (m, 1H), 3.21-3.12 (m, 2H), 2.97-2.93 (m, 2H), 2.77-2.69 (m, 3H), 1.80-1.71 (m, 2H), 1.69-1.57 (m, 4H), 0.94-0.90 (m, 3H).

### Synthesis of 4-[2-tert-butyl-4-(3-{[ethyl(methyl)sulfamoyl]amino}-2-fluorophenyl)-1,3-thiazol-5-yl]-N-(4-{3,8-diazabicyclo[3.2.1]octan-8-yl}-3-fluorophenyl)pyrimidin-2-amine; Trifluoroacetic Acid Salt

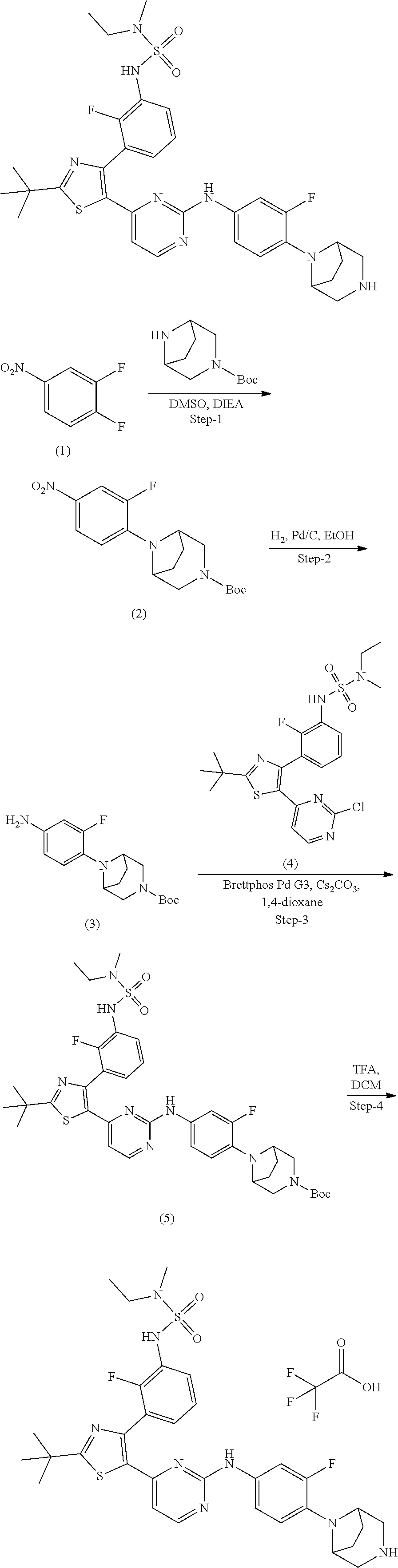

### Step-1: Synthesis of tert-butyl 8-(2-fluoro-4-nitrophenyl)-3,8-diazabicyclo[3.2.1]octane-3-carboxylate (2)

To a mixture of 1,2-difluoro-4-nitrobenzene (1.0 g, 6.286 mmol, 1 equiv) and DIEA (2.44 g, 18.858 mmol, 3.0 equiv) in DMSO (10 mL) was added tert-butyl 3,8-diazabicyclo[3.2.1]octane-3-carboxylate (1.33 g, 6.286 mmol, 1.0 equiv) in portions at room temperature. The resulting mixture was stirred for 3 h at 100° C. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with CH₂Cl₂:MeOH (10:1) to afford tert-butyl 8-(2-fluoro-4-nitrophenyl)-3,8-diazabicyclo[3.2.1]octane-3-carboxylate (1.2 g, 54.33%) as a yellow solid. LCMS: C₁₇H₂₂FN₃O₄ requires: 351.2, found: m/z=352.2 [M+H]⁺.

### Step-2: Synthesis of tert-butyl 8-(4-amino-2-fluorophenyl)-3,8-diazabicyclo[3.2.1]octane-3-carboxylate (3)

To a mixture of tert-butyl 8-(2-fluoro-4-nitrophenyl)-3,8-diazabicyclo[3.2.1]octane-3-carboxylate (600 mg, 1.708 mmol, 1 equiv) in EtOH (10 mL) was added Pd/C (299.84 mg, 2.818 mmol, 1.65 equiv) in portions at room temperature. The resulting mixture was stirred for 3 h at room temperature under a hydrogen atmosphere. The resulting mixture was filtered. The filtrate was concentrated under reduced pressure. The residue was purified by silica gel column chromatography eluted with CH₂Cl₂:MeOH (10:1) to afford tert-butyl 8-(4-amino-2-fluorophenyl)-3,8-diazabicyclo[3.2.1]octane-3-carboxylate (400 mg, 72.89%) as a yellow solid. LCMS: C₁₇H₂₄FN₃O₂ requires: 321.2, found: m/z=322.2 [M+H]⁺.

### Step-3: Synthesis of tert-butyl 8-[4-({4-[2-tert-butyl-4-(3-{[ethyl(methyl)sulfamoyl]amino}-2-fluorophenyl)-1,3-thiazol-5-yl]pyrimidin-2-yl}amino)-2-fluorophenyl]-3,8-diazabicyclo[3.2.1]octane-3-carboxylate (5)

To a mixture of tert-butyl 8-(4-amino-2-fluorophenyl)-3,8-diazabicyclo[3.2.1]octane-3-carboxylate (380 mg, 1.182 mmol, 1 equiv) and ({3-[2-tert-butyl-5-(2-chloropyrimidin-4-yl)-1,3-thiazol-4-yl]-2-fluorophenyl}sulfamoyl)(ethyl)methylamine (629.49 mg, 1.300 mmol, 1.1 equiv) in 1,4-dioxane (4 mL) was added BrettPhos Pd G₃ (107.18 mg, 0.118 mmol, 0.1 equiv) and Cs₂CO₃ (770.46 mg, 2.364 mmol, 2.0 equiv) at room temperature under a nitrogen atmosphere. The resulting mixture was stirred overnight at 80° C. under the nitrogen atmosphere. The resulting mixture was concentrated under reduced pressure. The residue was purified by silica gel column chromatography eluted with CH₂Cl₂:MeOH (5:1) to afford tert-butyl 8-[4-({4-[2-tert-butyl-4-(3-{[ethyl(methyl)sulfamoyl]amino}-2-fluorophenyl)-1,3-thiazol-5-yl]pyrimidin-2-yl}amino)-2-fluorophenyl]-3,8-diazabicyclo[3.2.1]octane-3-carboxylate (385 mg, 42.35%) as a yellow solid. LCMS: C₃₇H₄₆F₂N₈O₄S₂ requires: 768.3, found: m/z=769.3 [M+H]⁺.

### Step-4: Synthesis of 4-[2-tert-butyl-4-(3-{[ethyl(methyl)sulfamoyl]amino}-2-fluorophenyl)-1,3-thiazol-5-yl]-N-(4-{3,8-diazabicyclo[3.2.1]octan-8-yl}-3-fluorophenyl)pyrimidin-2-amine; Trifluoroacetic Acid

To a mixture of tert-butyl 8-[4-({4-[2-tert-butyl-4-(3-{[ethyl(methyl)sulfamoyl]amino}-2-fluorophenyl)-1,3-thiazol-5-yl]pyrimidin-2-yl}amino)-2-fluorophenyl]-3,8-diazabicyclo[3.2.1]octane-3-carboxylate (380 mg, 0.494 mmol, 1 equiv) in DCM (3 mL) was added TFA (3 mL) dropwise at room temperature. The resulting mixture was stirred for one hour at room temperature. The resulting mixture was concentrated under reduced pressure to afford 4-[2-tert-butyl-4-(3-{[ethyl(methyl)sulfamoyl]amino}-2-fluorophenyl)-1,3-thiazol-5-yl]-N-(4-{3,8-diazabicyclo[3.2.1]octan-8-yl}-3-fluorophenyl)pyrimidin-2-amine; trifluoroacetic acid salt (350 mg, crude) as a red solid. LCMS: C₃₂H₃₈F₂N₈O₂S₂ requires: 668.3, found: m/z=669.3 [M+H]⁺. ¹H NMR (300 MHz, CD₃OD) δ 8.24 (d, J=4.8 Hz, 1H), 7.72-7.62 (m, 2H), 7.32 (d, J=8.4 Hz, 2H), 7.20 (d, J=8.7 Hz, 1H), 6.95-7.82 (m, 1H), 6.60-6.50 (m, 1H), 4.28 (s, 2H), 3.49-3.38 (m, 2H), 3.28-3.17 (m, 4H), 2.82-2.76 (m, 3H), 2.32-2.21 (m, 2H), 2.09-1.98 (m, 2H), 1.58-1.52 (m, 9H), 1.18-1.02 (m, 3H).

### Synthesis of N-{3-[5-(2-aminopyrimidin-4-yl)-2-(4-methylpiperidin-4-yl)-1,3-thiazol-4-yl]-2-fluorophenyl}propane-1-sulfonamide

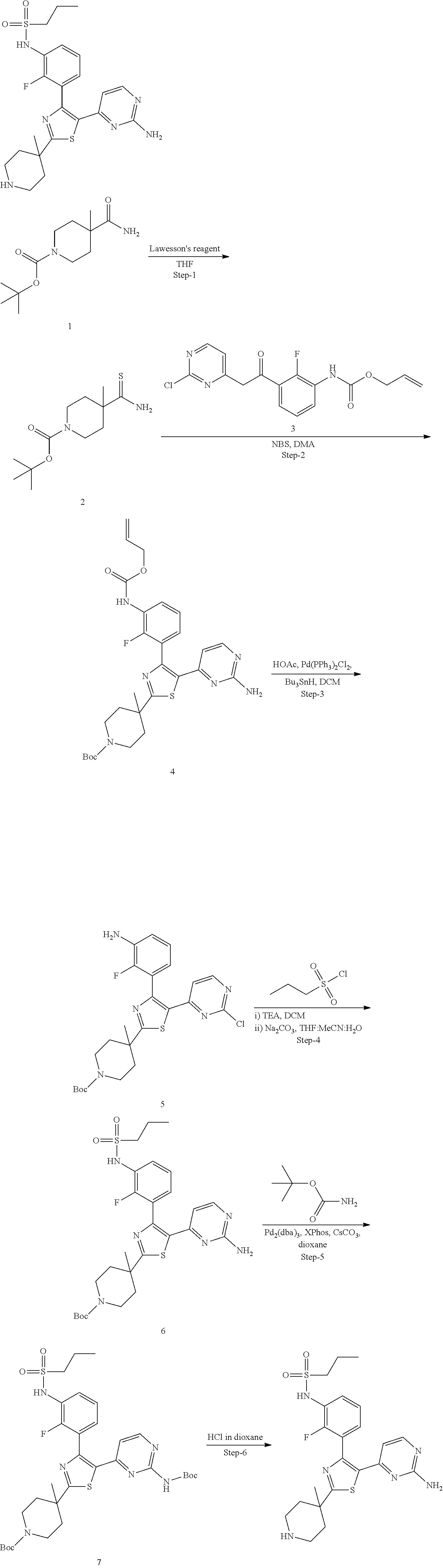

### Step-1: Synthesis of tert-butyl 4-carbamothioyl-4-methylpiperidine-1-carboxylate (2)

To a mixture of tert-butyl 4-carbamoyl-4-methylpiperidine-1-carboxylate (4 g, 16.5 mmol, 1 equiv) in THF (40 mL) was added Lawesson's Reagent (3.34 g, 8.25 mmol, 0.5 equiv) at room temperature. The resulting mixture was stirred overnight at 50° C. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with PE:EtOAc (3:1) to afford tert-butyl 4-carbamothioyl-4-methylpiperidine-1-carboxylate (2.1 g, 44.31%) as a yellow oil. LCMS: C₁₂H₂₂N₂O₂S requires: 258.1, found: m/z=259.0 [M+H]⁺.

### Step-2: Synthesis of tert-butyl 4-[5-(2-chloropyrimidin-4-yl)-4-(2-fluoro-3-{[(prop-2-en-1-yloxy)carbonyl]amino}phenyl)-1,3-thiazol-2-yl]-4-methylpiperidine-1-carboxylate (4)

To a mixture of prop-2-en-1-yl N-{3-[2-(2-chloropyrimidin-4-yl)acetyl]-2-fluorophenyl}carbamate (3 g, 8.57 mmol, 1 equiv) in DMA (30 mL) was added NBS (1.53 g, 8.57 mmol, 1 equiv) and tert-butyl 4-carbamothioyl-4-methylpiperidine-1-carboxylate (2.66 g, 10.29 mmol, 1.2 equiv) at room temperature. The resulting mixture was stirred for 2 h at 60° C. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with PE:EtOAc (3:1) to afford tert-butyl 4-[5-(2-chloropyrimidin-4-yl)-4-(2-fluoro-3-{[(prop-2-en-1-yloxy)carbonyl]amino}phenyl)-1,3-thiazol-2-yl]-4-methylpiperidine-1-carboxylate (2.1 g, 36.63%) as a yellow solid. LCMS: C₂₈H₃₁ClFN₅O₄S requires: 587.2, found: m/z=588.1 [M+H]⁺.

### Step-3: Synthesis of tert-butyl 4-(4-(3-amino-2-fluorophenyl)-5-(2-chloropyrimidin-4-yl)thiazol-2-yl)-4-methylpiperidine-1-carboxylate (5)

To a mixture of tert-butyl 4-[5-(2-chloropyrimidin-4-yl)-4-(2-fluoro-3-{[(prop-2-en-1-yloxy)carbonyl]amino}phenyl)-1,3-thiazol-2-yl]-4-methylpiperidine-1-carboxylate (2.1 g, 3.57 mmol, 1 equiv) and Pd(PPh₃)₂Cl₂ (50.13 mg, 0.07 mmol, 0.02 equiv) in DCM (30 mL) was added HOAc (514.6 mg, 8.57 mmol, 2.4 equiv) and n-Bu₃SnH (1.56 g, 5.35 mmol, 1.5 equiv) at 0° C. under a nitrogen atmosphere. The resulting mixture was stirred for 0.5 h at 0° C. under the nitrogen atmosphere. The resulting mixture was concentrated under reduced pressure. The residue was purified by silica gel column chromatography eluted with PE:EtOAc (1:1) to afford tert-butyl 4-(4-(3-amino-2-fluorophenyl)-5-(2-chloropyrimidin-4-yl)thiazol-2-yl)-4-methylpiperidine-1-carboxylate (1.6 g, 92.82%) as a yellow oil. LCMS: C₂₄H₂₇ClFN₅O₂S requires: 503.2, found: m/z=504.1 [M+H]⁺.

### Step-4: Synthesis of tert-butyl 4-(5-(2-chloropyrimidin-4-yl)-4-(2-fluoro-3-(propylsulfonamido)phenyl)thiazol-2-yl)-4-methylpiperidine-1-carboxylate (6)

To a mixture of tert-butyl 4-[4-(3-amino-2-fluorophenyl)-5-(2-chloropyrimidin-4-yl)-1,3-thiazol-2-yl]-4-methylpiperidine-1-carboxylate (1.6 g, 3.17 mmol, 1 equiv) in DCM (20 mL) was added TEA (963.7 mg, 9.52 mmol, 3 equiv) and propane-1-sulfonyl chloride (679.0 mg, 4.76 mmol, 1.5 equiv) at 0° C. The resulting mixture was stirred for 2 h at room temperature. The resulting mixture was concentrated under reduced pressure. To the crude product in THF (10 mL), MeCN (10 mL), and H₂O (10 mL) was added Na₂CO₃ (2 g, 19.25 mmol, 10 equiv) at room temperature. The resulting mixture was stirred overnight at 50° C. The resulting mixture was extracted with DCM. The combined organic layers were washed with water and dried over anhydrous Na₂SO₄. After filtration, the filtrate was concentrated under reduced pressure. The residue was purified by silica gel column chromatography eluted with PE:EtOAc (5:1) to afford tert-butyl 4-[5-(2-chloropyrimidin-4-yl)-4-[2-fluoro-3-(propane-1-sulfonamido)phenyl]-1,3-thiazol-2-yl]-4-methylpiperidine-1-carboxylate (1.1 g, 92.38%) as a yellow solid. LCMS: C₂₇H₃₃ClFN₅O₄S₂ requires: 609.2, found: m/z=610.1 [M+H]⁺.

### Step-5: Synthesis of tert-butyl 4-(5-{2-[(tert-butoxycarbonyl)amino]pyrimidin-4-yl}-4-[2-fluoro-3-(propane-1-sulfonamido)phenyl]-1,3-thiazol-2-0l)-4-methylpiperidine-1-carboxylate (7)

To a mixture of tert-butyl 4-[5-(2-chloropyrimidin-4-yl)-4-[2-fluoro-3-(propane-1-sulfonamido)phenyl]-1,3-thiazol-2-yl]-4-methylpiperidine-1-carboxylate (1 g, 1.63 mmol, 1 equiv) and Pd₂(dba)₃ (150 mg, 0.16 mmol, 0.1 equiv) in dioxane (10 mL) was added XPhos (156.2 mg, 0.32 mmol, 0.2 equiv), Cs₂CO₃ (1.1 g, 3.27 mmol, 2 equiv) and tert-butyl carbamate (287.9 mg, 2.45 mmol, 1.5 equiv) at room temperature under a nitrogen atmosphere. The resulting mixture was stirred overnight at 80° C. The resulting mixture was concentrated under reduced pressure. The residue was purified by silica gel column chromatography eluted with PE:EtOAc (9:1) to afford tert-butyl 4-(5-{2-[(tert-butoxycarbonyl)amino]pyrimidin-4-yl}-4-[2-fluoro-3-(propane-1-sulfonamido)phenyl]-1,3-thiazol-2-yl)-4-methylpiperidine-1-carboxylate (1 g, 77.72%) as a brown solid. LCMS: C₃₂H₄₃FN₆O₆S₂ requires: 690.3, found: m/z=691.2 [M+H]⁺.

### Step-6: Synthesis of N-{3-[5-(2-aminopyrimidin-4-yl)-2-(4-methylpiperidin-4-yl)-1,3-thiazol-4-yl]-2-fluorophenyl}propane-1-sulfonamide

A mixture of tert-butyl 4-(5-{2-[(tert-butoxycarbonyl)amino]pyrimidin-4-yl}-4-[2-fluoro-3-(propane-1-sulfonamido)phenyl]-1,3-thiazol-2-yl)-4-methylpiperidine-1-carboxylate (1 g, 1.44 mmol, 1 equiv) in HCl/1,4-dioxane (10 mL, 4 M) was stirred overnight at room temperature. The resulting mixture was concentrated under reduced pressure. The residue was purified by reverse phase flash chromatography with H₂O:MeCN (1:3) to afford N-{3-[5-(2-aminopyrimidin-4-yl)-2-(4-methylpiperidin-4-yl)-1,3-thiazol-4-yl]-2-fluorophenyl}propane-1-sulfonamide (614.8 mg, 84.49%) as a yellow solid. LCMS: C₂₂H₂₇FN₆O₂S₂ requires: 490.2, found: m/z=491.1 [M+H]⁺.

¹H NMR (400 MHz, CD₃OD) δ 8.11 (d, J=6.4 Hz, 1H), 7.71-7.67 (m, 1H), 7.53-7.50 (m, 1H), 7.40-7.36 (m, 1H), 6.67-6.64 (m, 1H), 3.42-3.35 (m, 3H), 3.32-3.30 (m, 1H), 3.18-3.14 (m, 2H), 2.56-2.50 (m, 2H), 2.15-2.08 (m, 2H), 1.90-1.84 (m, 2H), 1.62 (s, 3H), 1.08-1.04 (m, 3H).

### Synthesis of N-{3-[5-(2-aminopyrimidin-4-yl)-2-{2-azaspiro[3.5]nonan-7-yl}-1,3-thiazol-4-yl]-2-fluorophenyl}propane-1-sulfonamide; Trifluoroacetic Acid Salt

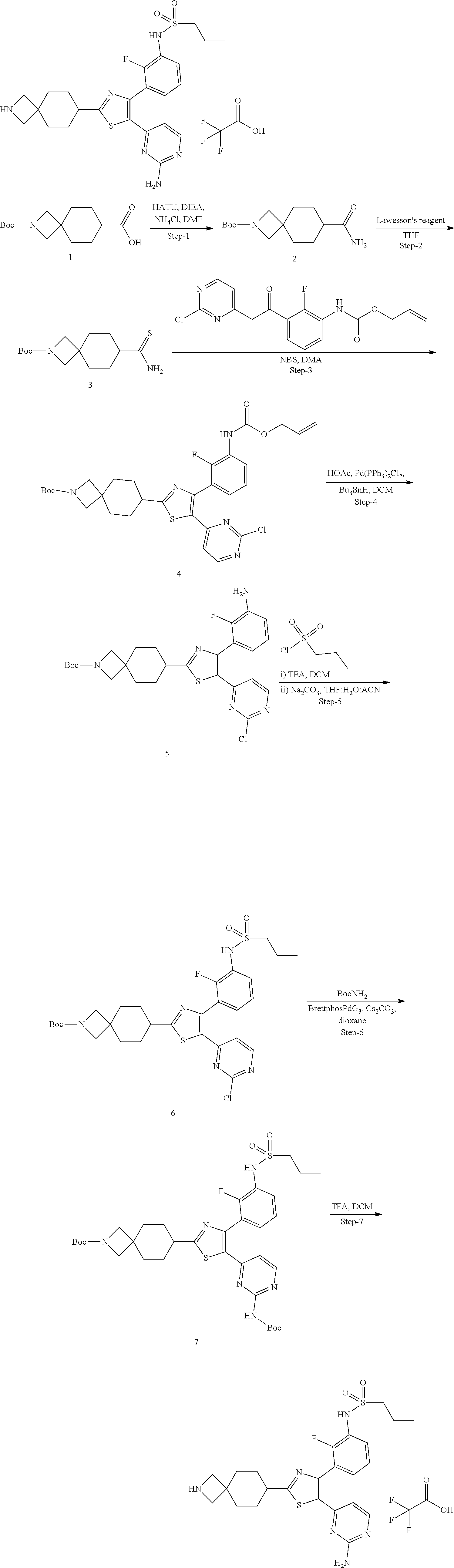

### Step-1: Synthesis of tert-butyl 7-carbamoyl-2-azaspiro[3.5]nonane-2-carboxylate (2)

To a mixture of 2-(tert-butoxycarbonyl)-2-azaspiro[3.5]nonane-7-carboxylic acid (4 g, 14.85 mmol, 1 equiv) and HATU (8.47 g, 22.28 mmol, 1.5 equiv) in DMF (40 mL) was added NH₄Cl (3.97 g, 74.26 mmol, 5 equiv) and the mixture was stirred for 15 min. Then, DIEA (5.76 g, 44.55 mmol, 3 equiv) was added. The resulting mixture was stirred at room temperature for one hour. The residue was purified by reverse phase flash chromatography eluted with ACN:H₂O (1:1) to afford tert-butyl 7-carbamoyl-2-azaspiro[3.5]nonane-2-carboxylate (3.5 g, 87.82%) as a white solid. LCMS: C₁₄H₂₄N₂O₃ requires: 268.2, found: m/z=269.3 [M+H]⁺.

### Step-2: Synthesis of tert-butyl 7-carbamothioyl-2-azaspiro[3.5]nonane-2-carboxylate (3)

To a mixture of tert-butyl 7-carbamoyl-2-azaspiro[3.5]nonane-2-carboxylate (3.5 g, 13.04 mmol, 1 equiv) in THF (40 mL) was added Lawesson's Reagent (2.64 g, 6.53 mmol, 0.5 equiv). The resulting mixture was stirred at room temperature overnight under a nitrogen atmosphere. The resulting mixture was concentrated under vacuum. The residue was purified by silica gel column chromatography eluted with DCM:MeOH (10:1) to afford tert-butyl 7-carbamothioyl-2-azaspiro[3.5]nonane-2-carboxylate (2.5 g, 51.89%) as a white solid. LCMS: C₁₄H₂₄N₂O₂S requires: 284.2, found: m/z=285.2 [M+H]⁺.

### Step-3: Synthesis of tert-butyl 7-[5-(2-chloropyrimidin-4-yl)-4-(2-fluoro-3-{[(prop-2-en-1-yloxy)carbonyl]amino}phenyl)-1,3-thiazol-2-yl]-2-azaspiro[3.5]nonane-2-carboxylate (4)

To a mixture of prop-2-en-1-yl N-{3-[2-(2-chloropyrimidin-4-yl)acetyl]-2-fluorophenyl}carbamate (3.07 g, 8.78 mmol, 1 equiv) in DMA (20 mL) was added NBS (1.56 g, 8.78 mmol, 1 equiv) and the mixture was stirred for 15 min. Then, tert-butyl 7-carbamothioyl-2-azaspiro[3.5]nonane-2-carboxylate (2.5 g, 8.79 mmol, 1 equiv) was added. The resulting mixture was stirred at 60° C. for 2 h. The residue was purified by reverse phase flash chromatography eluted with ACN:H₂O (1:1) to afford tert-butyl 7-[5-(2-chloropyrimidin-4-yl)-4-(2-fluoro-3-{[(prop-2-en-1-yloxy)carbonyl]amino}phenyl)-1,3-thiazol-2-yl]-2-azaspiro[3.5]nonane-2-carboxylate (1.4 g, 19.48%) as a yellow solid. LCMS: C₃₀H₃₃ClFN₅O₄S requires: 613.2, found: m/z=614.2 [M+H]⁺.

### Step-4: Synthesis of tert-butyl 7-[4-(3-amino-2-fluorophenyl)-5-(2-chloropyrimidin-4-yl)-1,3-thiazol-2-yl]-2-azaspiro[3.5]nonane-2-carboxylate (5)

To a mixture of tert-butyl 7-[5-(2-chloropyrimidin-4-yl)-4-(2-fluoro-3-{[(prop-2-en-1-yloxy)carbonyl]amino}phenyl)-1,3-thiazol-2-yl]-2-azaspiro[3.5]nonane-2-carboxylate (1.4 g, 2.28 mmol, 1 equiv) in DCM (10 mL) was added HOAc (329 mg, 5.47 mmol, 2.4 equiv) and Pd(PPh₃)₂Cl₂ (32 mg, 0.046 mmol, 0.02 equiv). Then, n-Bu₃SnH (995 mg, 3.42 mmol, 1.5 equiv) was added at 0° C. under a nitrogen atmosphere. The resulting mixture was stirred at room temperature for one hour. The reaction was quenched with saturated aqueous NaHCO₃ at 0° C. The resulting mixture was concentrated under vacuum. The residue was purified by silica gel column chromatography eluted with PE:EtOAc (1:1) to afford tert-butyl 7-[4-(3-amino-2-fluorophenyl)-5-(2-chloropyrimidin-4-yl)-1,3-thiazol-2-yl]-2-azaspiro[3.5]nonane-2-carboxylate (750 mg, 58.96%) as a yellow solid. LCMS: C₂₆H₂₉ClFN₅O₂S requires: 529.2, found: m/z=530.4 [M+H]⁺.

### Step-5: Synthesis of tert-butyl 7-[5-(2-chloropyrimidin-4-yl)-4-[2-fluoro-3-(propane-1-sulfonamido)Phenyl]-1,3-thiazol-2-yl]-2-azaspiro[3.5]nonane-2-carboxylate

To a mixture of tert-butyl 7-[4-(3-amino-2-fluorophenyl)-5-(2-chloropyrimidin-4-yl)-1,3-thiazol-2-yl]-2-azaspiro[3.5]nonane-2-carboxylate (750 mg, 1.42 mmol, 1 equiv) and TEA (430 mg, 4.25 mmol, 3 equiv) in DCM (10 mL) was added propane-1-sulfonyl chloride (605 mg, 4.25 mmol, 3 equiv) at 0° C. The resulting mixture was stirred at room temperature for one hour. The resulting mixture was concentrated under vacuum. To the above mixture was added THF (10 mL), ACN (10 mL), and Na₂CO₃ (2249 mg, 21.22 mmol, 15 equiv) in H₂O (10 mL). The resulting mixture was stirred at 50° C. overnight. The resulting mixture was concentrated under vacuum. The residue was purified by silica gel column chromatography eluted with PE:EtOAc (1:1) to afford tert-butyl 7-[5-(2-chloropyrimidin-4-yl)-4-[2-fluoro-3-(propane-1-sulfonamido)phenyl]-1,3-thiazol-2-yl]-2-azaspiro[3.5]nonane-2-carboxylate (750 mg, 79.15%) as a yellow solid. LCMS: C₂₉H₃₅ClFN₅O₄S₂ requires: 635.2, found: m/z=636.2 [M+H]⁺.

### Step-6: Synthesis of tert-butyl 7-(5-{2-[(tert-butoxycarbonyl)amino]pyrimidin-4-yl}-4-[2-fluoro-3-(propane-1-sulfonamido)phenyl]-1,3-thiazol-2-yl)-2-azaspiro[3.5]nonane-2-carboxylate (7)

To a mixture of tert-butyl 7-[5-(2-chloropyrimidin-4-yl)-4-[2-fluoro-3-(propane-1-sulfonamido)phenyl]-1,3-thiazol-2-yl]-2-azaspiro[3.5]nonane-2-carboxylate (750 mg, 1.18 mmol, 1 equiv) and tert-butyl carbamate (691 mg, 5.9 mmol, 5 equiv) in 1,4-dioxane (10 mL) was added Cs₂CO₃ (768 mg, 2.36 mmol, 2 equiv) and BrettPhos Pd G3 (107 mg, 0.12 mmol, 0.1 equiv). The resulting mixture was stirred at 80° C. for one hour under a nitrogen atmosphere. The resulting mixture was concentrated under vacuum. The residue was purified by silica gel column chromatography eluted with DCM:MeOH (10:1) to afford tert-butyl 7-(5-{2-[(tert-butoxycarbonyl)amino]pyrimidin-4-yl}-4-[2-fluoro-3-(propane-1-sulfonamido)phenyl]-1,3-thiazol-2-yl)-2-azaspiro[3.5]nonane-2-carboxylate (800 mg, 85.19%) as a yellow solid. LCMS: C₃₄H₄₅FN₆O₆S₂ requires: 716.3, found: m/z=717.2 [M+H]⁺.

### Step-7: Synthesis of N-{3-[5-(2-aminopyrimidin-4-yl)-2-{2-azaspiro[3.5]nonan-7-yl}-1,3-thiazol-4-yl]-2-fluorophenyl}propane-1-sulfonamide; Trifluoroacetic Acid

To a mixture of tert-butyl 7-(5-{2-[(tert-butoxycarbonyl)amino]pyrimidin-4-yl}-4-[2-fluoro-3-(propane-1-sulfonamido)phenyl]-1,3-thiazol-2-yl)-2-azaspiro[3.5]nonane-2-carboxylate (500 mg, 0.7 mmol, 1 equiv) in DCM (6 mL) was added TFA (2 mL) at 0° C. The resulting mixture was stirred at 0° C. for one hour. The resulting mixture was concentrated under vacuum. The residue was purified by reverse phase flash chromatography eluted with ACN:H₂O (1:1) to afford N-{3-[5-(2-aminopyrimidin-4-yl)-2-{2-azaspiro[3.5]nonan-7-yl}-1,3-thiazol-4-yl]-2-fluorophenyl}propane-1-sulfonamide; trifluoroacetic acid salt (211 mg, 47.54%) as a yellow solid. LCMS: C₂₄H₂₉FN₆O₂S₂ requires: 516.2, found: m/z=517.2 [M+H]⁺.

¹H NMR (400 MHz, DMSO-d₆) δ 8.05 (d, J=5.2 Hz, 1H), 7.51-7.42 (m, 1H), 7.13-7.05 (m, 1H), 6.92-6.84 (m, 1H), 6.71 (s, 2H), 6.14 (d, J=5.2 Hz, 1H), 3.53 (s, 2H), 3.45 (s, 2H), 2.97 (s, 1H), 2.91-2.82 (m, 2H), 2.08-1.97 (m, 4H), 1.74-1.61 (m, 2H), 1.60-1.41 (m, 4H), 0.97-0.83 (m, 3H).

### Synthesis of N-{3-[5-(2-aminopyrimidin-4-yl)-2-{3-azaspiro[5.5]undecan-9-yl}-1,3-thiazol-4-yl]-2-fluorophenyl}propane-1-sulfonamide

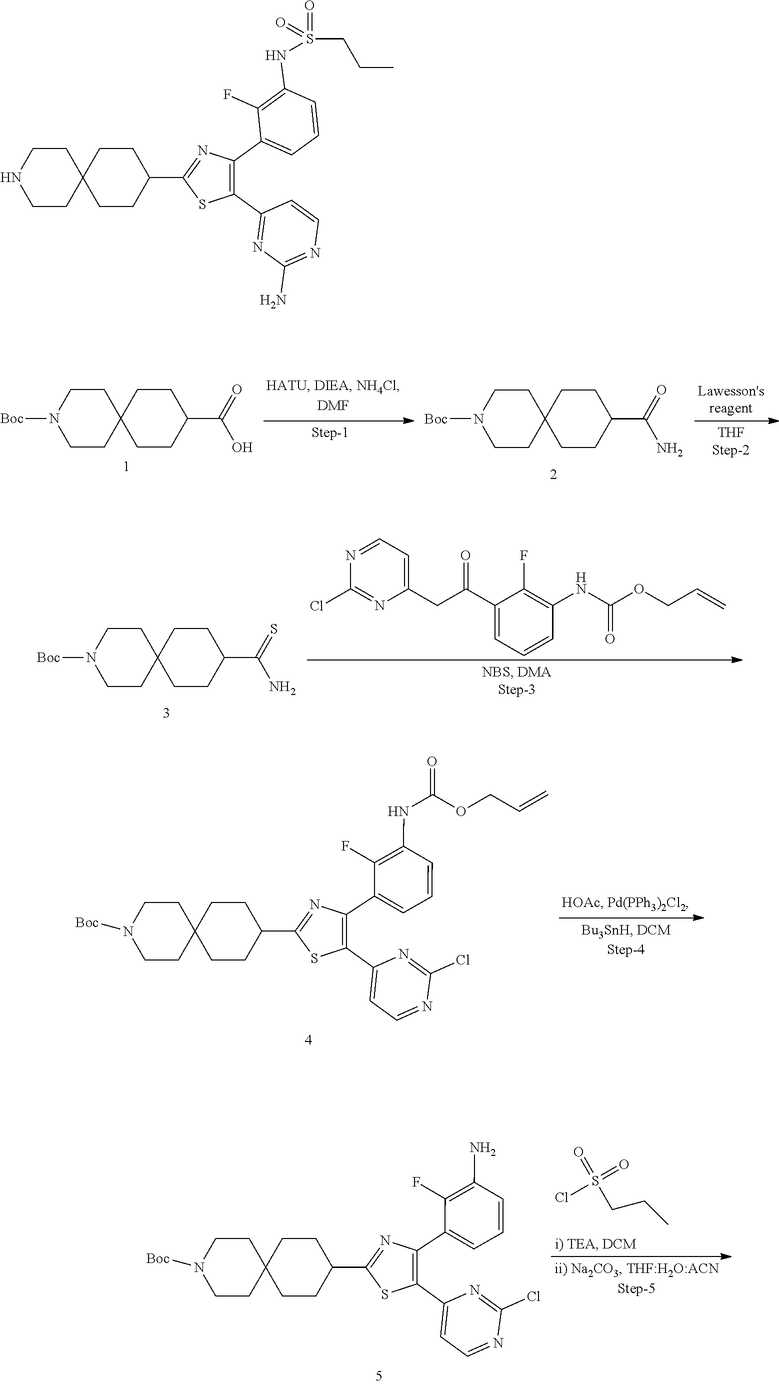

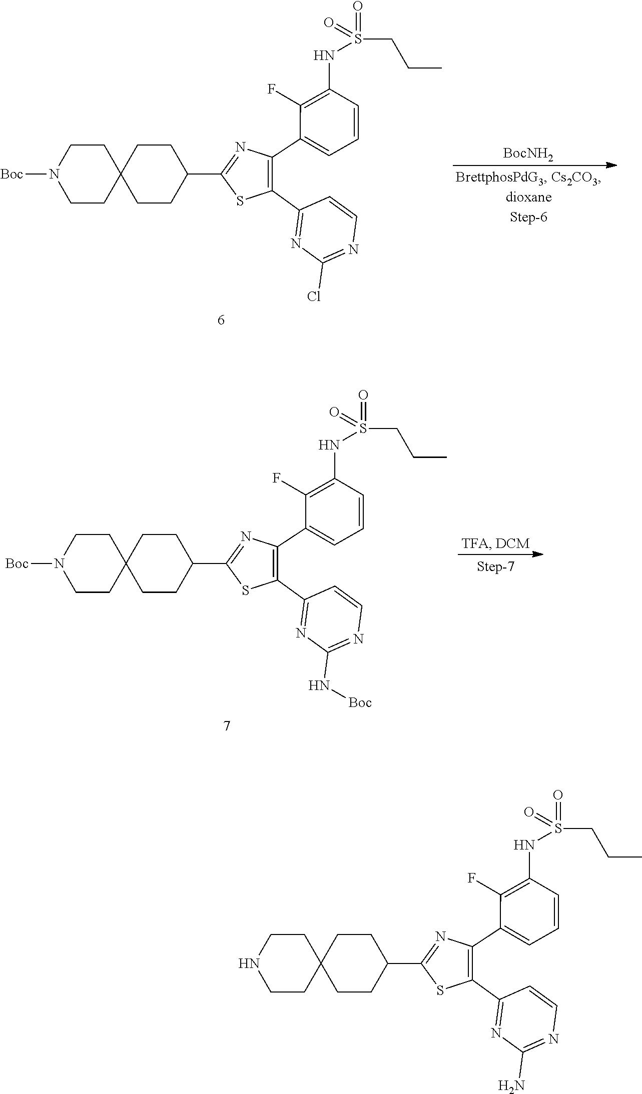

### Step-1: Synthesis of tert-butyl 9-carbamoyl-3-azaspiro[5.5]undecane-3-carboxylate (2)

To a mixture of 3-(tert-butoxycarbonyl)-3-azaspiro[5.5]undecane-9-carboxylic acid (3 g, 10.09 mmol, 1 equiv) and HATU (5.75 g, 15.13 mmol, 1.5 equiv) in DMF (30 mL) was added NH₄Cl (2.7 g, 50.44 mmol, 5 equiv) and the mixture was stirred for 15 min. Then, DIEA (3.91 g, 30.26 mmol, 3 equiv) was added. The resulting mixture was stirred at room temperature for one hour. The residue was purified by reverse phase flash chromatography eluted with ACN:H₂O (1:1) to afford tert-butyl 9-carbamoyl-3-azaspiro[5.5]undecane-3-carboxylate (2.1 g, 70.23%) as a yellow solid. LCMS: C₁₆H₂₈N₂O₃ requires: 296.2, found: m/z=297.2 [M+H]⁺.

### Step-2: Synthesis of tert-butyl 9-carbamothioyl-3-azaspiro[5.5]undecane-3-carboxylate (3)

To a mixture of tert-butyl 9-carbamoyl-3-azaspiro[5.5]undecane-3-carboxylate (2.2 g, 7.42 mmol, 1 equiv) in THF (30 mL) was added Lawesson's Reagent (1.5 g, 3.71 mmol, 0.5 equiv). The resulting mixture was stirred at room temperature overnight under a nitrogen atmosphere. The resulting mixture was concentrated under vacuum. The residue was purified by silica gel column chromatography eluted with PE:EtOAc (2:1) to afford tert-butyl 9-carbamothioyl-3-azaspiro[5.5]undecane-3-carboxylate (1.43 g, 55.49%) as an off-white solid. LCMS: C₁₆H₂₈N₂O₂S requires: 312.2, found: m/z=313.3 [M+H]⁺.

### Step-3: Synthesis of tert-butyl 9-[5-(2-chloropyrimidin-4-yl)-4-(2-fluoro-3-{[(prop-2-en-1-yloxy)carbonyl]amino}phenyl)-1,3-thiazol-2-yl]-3-azaspiro[5.5]undecane-3-carboxylate (4)

To a mixture of prop-2-en-1-yl N-{3-[2-(2-chloropyrimidin-4-yl)acetyl]-2-fluorophenyl}carbamate (1.3 g, 3.72 mmol, 1 equiv) in DMA (10 mL) was added NBS (662 mg, 3.72 mmol, 1 equiv) and the mixture was stirred for 15 min. Then, tert-butyl 9-carbamothioyl-3-azaspiro[5.5]undecane-3-carboxylate (1.51 g, 4.83 mmol, 1.3 equiv) was added. The resulting mixture was stirred at 60° C. for 2 h. The residue was purified by reverse phase flash chromatography eluted with ACN:H₂O (1:1) to afford tert-butyl 9-[5-(2-chloropyrimidin-4-yl)-4-(2-fluoro-3-{[(prop-2-en-1-yloxy)carbonyl]amino}phenyl)-1,3-thiazol-2-yl]-3-azaspiro [5.5]undecane-3-carboxylate (1.14 g, 38.68%) as a yellow solid. LCMS: C₃₂H₃₇ClFN₅O₄S requires: 641.2, found: m/z=642.4 [M+H]⁺.

### Step-4: Synthesis of tert-butyl 9-[4-(3-amino-2-fluorophenyl)-5-(2-chloropyrimidin-4-yl)-1,3-thiazol-2-yl]-3-azaspiro[5.5]undecane-3-carboxylate (5)

To a mixture of tert-butyl 9-[5-(2-chloropyrimidin-4-yl)-4-(2-fluoro-3-{[(prop-2-en-1-yloxy)carbonyl]amino}phenyl)-1,3-thiazol-2-yl]-3-azaspiro[5.5]undecane-3-carboxylate (1.3 g, 2.02 mmol, 1 equiv) in DCM (10 mL) was added HOAc (0.29 g, 4.83 mmol, 2.4 equiv) and Pd(PPh₃)₂Cl₂ (28 mg, 0.04 mmol, 0.02 equiv). Then, n-Bu₃SnH (884 mg, 3.04 mmol, 1.5 equiv) was added at 0° C. under a nitrogen atmosphere. The resulting mixture was stirred at room temperature for one hour. The reaction was quenched with saturated aqueous NaHCO₃ at 0° C. The resulting mixture was concentrated under vacuum. The residue was purified by silica gel column chromatography eluted with PE:EtOAc (1:1) to afford tert-butyl 9-[4-(3-amino-2-fluorophenyl)-5-(2-chloropyrimidin-4-yl)-1,3-thiazol-2-yl]-3-azaspiro[5.5]undecane-3-carboxylate (1.02 g, 90%) as a yellow solid. LCMS: C₂₈H₃₃ClFN₅O₂S requires: 557.2, found: m/z=558.2 [M+H]⁺.

### Step-5: Synthesis of tert-butyl 9-[5-(2-chloropyrimidin-4-yl)-4-[2-fluoro-3-(propane-1-sulfonamido)Phenyl]-1,3-thiazol-2-yl]-3-azaspiro[5.5]undecane-3-carboxylate (6)

To a mixture of tert-butyl 9-[4-(3-amino-2-fluorophenyl)-5-(2-chloropyrimidin-4-yl)-1,3-thiazol-2-yl]-3-azaspiro[5.5]undecane-3-carboxylate (1 g, 1.79 mmol, 1 equiv) and TEA (0.54 g, 5.38 mmol, 3 equiv) in DCM (15 mL) was added propane-1-sulfonyl chloride (0.38 g, 2.69 mmol, 1.5 equiv) at 0° C. The resulting mixture was stirred at room temperature for one hour. The resulting mixture was concentrated under vacuum. To the above mixture was added THF (10 mL), ACN (10 mL), and Na₂CO₃ (2.85 g, 26.85 mmol, 15 equiv) in H₂O (10 mL). The resulting mixture was stirred at 50° C. overnight. The resulting mixture was concentrated under vacuum. The residue was purified by silica gel column chromatography eluted with PE:EtOAc (1:1) to afford tert-butyl 9-[5-(2-chloropyrimidin-4-yl)-4-[2-fluoro-3-(propane-1-sulfonamido)phenyl]-1,3-thiazol-2-yl]-3-azaspiro[5.5]undecane-3-carboxylate (1.12 g, 89.40%) as a yellow solid. LCMS: C₃₁H₃₉ClFN₅O₄S₂ requires: 663.2, found: m/z=664.2 [M+H]⁺.

### Step-6: Synthesis of tert-butyl 9-(5-{2-[(tert-butoxycarbonyl)amino]pyrimidin-4-yl}-4-[2-fluoro-3-(propane-1-sulfonamido)phenyl]-1,3-thiazol-2-yl)-3-azaspiro[5.5]undecane-3-carboxylate (7)

To a mixture of tert-butyl 9-[5-(2-chloropyrimidin-4-yl)-4-[2-fluoro-3-(propane-1-sulfonamido)phenyl]-1,3-thiazol-2-yl]-3-azaspiro[5.5]undecane-3-carboxylate (1.1 g, 1.66 mmol, 1 equiv) and tert-butyl carbamate (0.97 g, 8.28 mmol, 5 equiv) in 1,4-dioxane (10 mL) was added Cs₂CO₃ (1.08 g, 3.32 mmol, 2 equiv) and BrettPhos Pd G3 (150 mg, 0.17 mmol, 0.1 equiv). The resulting mixture was stirred at 80° C. for one hour under a nitrogen atmosphere. The resulting mixture was concentrated under vacuum. The residue was purified by silica gel column chromatography eluted with PE:EtOAc (1:10) to afford tert-butyl 9-(5-{2-[(tert-butoxycarbonyl)amino]pyrimidin-4-yl}-4-[2-fluoro-3-(propane-1-sulfonamido)phenyl]-1,3-thiazol-2-yl)-3-azaspiro [5.5]undecane-3-carboxylate (1.2 g, 84.63%) as a yellow solid. LCMS: C₃₆H₄₉FN₆O₆S₂ requires: 744.3, found: m/z=745.3 [M+H]⁺.

### Step-7: Synthesis of N-{3-[5-(2-aminopyrimidin-4-yl)-2-{3-azaspiro[5.5]undecan-9-yl}-1,3-thiazol-4-yl]-2-fluorophenyl}propane-1-sulfonamide

To a mixture of tert-butyl 9-(5-{2-[(tert-butoxycarbonyl)amino]pyrimidin-4-yl}-4-[2-fluoro-3-(propane-1-sulfonamido)phenyl]-1,3-thiazol-2-yl)-3-azaspiro[5.5]undecane-3-carboxylate (1.2 g, 1.61 mmol, 1 equiv) in DCM (12 mL) was added TFA (4 mL) at 0° C. The resulting mixture was stirred at 0° C. for one hour. The resulting mixture was concentrated under vacuum. The residue was purified by reverse phase flash chromatography eluted with ACN:H₂O (0.1% NH₄HCO₃) (1:1) to afford N-{3-[5-(2-aminopyrimidin-4-yl)-2-{3-azaspiro[5.5]undecan-9-yl}-1,3-thiazol-4-yl]-2-fluorophenyl}propane-1-sulfonamide (520.8 mg, 57.69%) as a light yellow solid. LCMS: C₂₆H₃₃FN₆O₂S₂ requires: 544.2, found: m/z=545.3 [M+H]⁺.

¹H NMR (400 MHz, CD₃OD) δ 8.04 (d, J=5.2 Hz, 1H), 7.70-7.60 (m, 1H), 7.34-7.21 (m, 2H), 6.28 (d, J=5.2 Hz, 1H), 3.19-2.91 (m, 7H), 2.09-2.00 (m, 2H), 1.98-1.67 (m, 8H), 1.57-1.50 (m, 2H), 1.46-1.34 (m, 2H), 1.05-0.97 (m, 3H).

### Synthesis of N-(3-(5-(2-aminopyrimidin-4-yl)-2-(piperidin-4-yl)thiazol-4-yl)-2-fluorophenyl)propane-1-sulfonamide Hydrochloride Salt

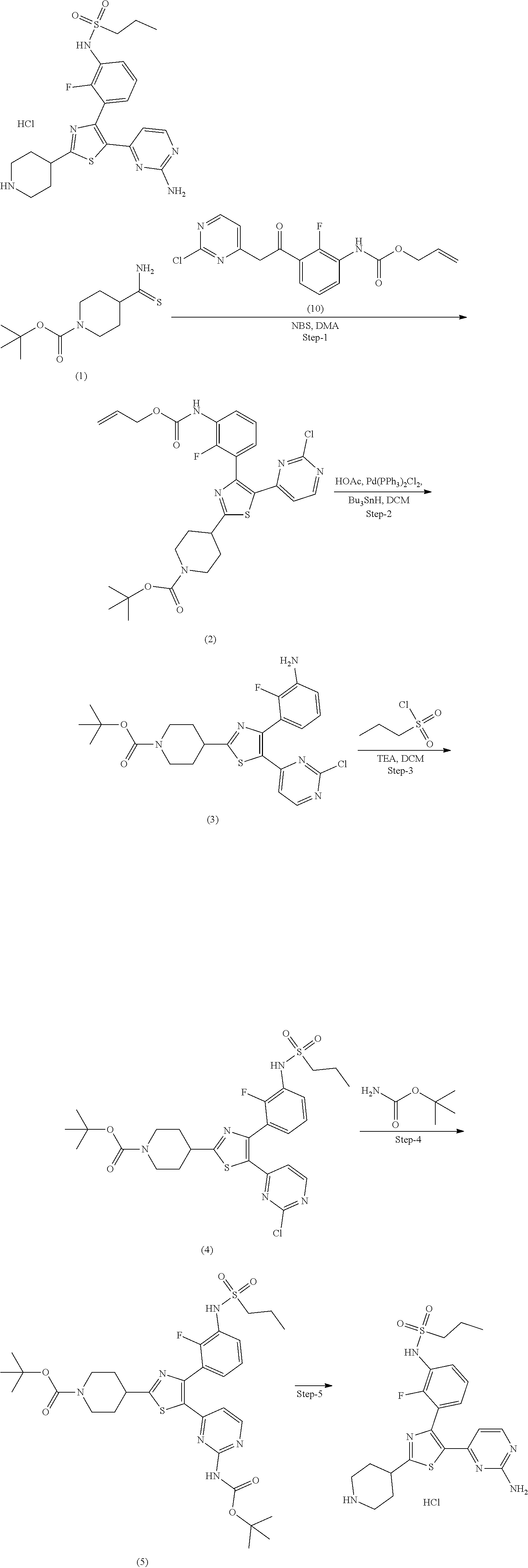

### Step-1: Synthesis of tert-butyl 4-[5-(2-chloropyrimidin-4-yl)-4-(2-fluoro-3-{[(prop-2-en-1-yloxy)carbonyl]amino}phenyl)-1,3-thiazol-2-yl}piperidine-1-carboxylate

To a mixture of prop-2-en-1-yl N-{3-[2-(2-chloropyrimidin-4-yl)acetyl]-2-fluorophenyl}carbamate (1.1 g, 3.145 mmol, 1.00 equiv) and NBS (559.8 mg, 3.145 mmol, 1.0 equiv) in DMA (13 mL) was added tert-butyl 4-carbamothioylpiperidine-1-carboxylate (922.2 mg, 3.774 mmol, 1.20 equiv) at room temperature. The resulting mixture was stirred for 2 h at 60° C. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with PE:EtOAc (1:1) to afford tert-butyl 4-[5-(2-chloropyrimidin-4-yl)-4-(2-fluoro-3-{[(prop-2-en-1-yloxy)carbonyl]amino}phenyl)-1,3-thiazol-2-yl]piperidine-1-carboxylate (580 mg, 32.12%) as a yellow solid. LCMS: C₂₇H₂₉ClFN₅O₄S requires: 573.2, found: m/z=574.2 [M+H]⁺.

### Step-2: Synthesis of tert-butyl 4-[4-(3-amino-2-fluorophenyl)-5-(2-chloropyrimidin-4-yl)-1,3-thiazol-2-yl]piperidine-1-carboxylate (3)

To a mixture of tert-butyl 4-[5-(2-chloropyrimidin-4-yl)-4-(2-fluoro-3-{[(prop-2-en-1-yloxy)carbonyl]amino}phenyl)-1,3-thiazol-2-yl]piperidine-1-carboxylate (600 mg, 1.045 mmol, 1 equiv) and HOAc (156.9 mg, 2.612 mmol, 2.5 equiv) in DCM (8 mL) was added Pd(PPh₃)₂Cl₂ (14.7 mg, 0.021 mmol, 0.02 equiv) and n-Bu₃SnH (486.7 mg, 1.672 mmol, 1.6 equiv) in portions at 0° C. The resulting mixture was stirred for 0.5 h at room temperature under a nitrogen atmosphere. The resulting mixture was concentrated under vacuum to afford tert-butyl 4-[4-(3-amino-2-fluorophenyl)-5-(2-chloropyrimidin-4-yl)-1,3-thiazol-2-yl]piperidine-1-carboxylate (510 mg, crude) as a red oil. The resulting mixture was used in the next step directly without further purification. LCMS: C₂₃H₂₅ClFN₅O₂S requires: 489.1, found: m/z=490.1 [M+H]⁺.

### Step-3: Synthesis of tert-butyl 4-(5-(2-chloropyrimidin-4-yl)-4-(2-fluoro-3-(propylsulfonamido)phenyl)thiazol-2-yl)piperidine-1-carboxylate (4)

To a mixture of tert-butyl 4-[4-(3-amino-2-fluorophenyl)-5-(2-chloropyrimidin-4-yl)-1,3-thiazol-2-yl]piperidine-1-carboxylate (2 g, 4.082 mmol, 1 equiv) and TEA (1.24 g, 12.246 mmol, 3 equiv) in DCM (20 mL) was added propane-1-sulfonyl chloride (873.08 mg, 6.123 mmol, 1.5 equiv) dropwise at 0° C. The resulting mixture was stirred for 2 h at room temperature. The resulting mixture was concentrated under vacuum. To the crude product in H₂O (7 mL), MeCN (7 mL), and THF (7 mL) was added Na₂CO₃ (1.06 g, 10.065 mmol, 3.0 equiv) at room temperature. The resulting mixture was stirred at 50° C. overnight. The resulting mixture was extracted with DCM. The combined organic layers were washed with water and dried over anhydrous Na₂SO₄. After filtration, the filtrate was concentrated under reduced pressure. The residue was purified by silica gel column chromatography eluted with CH₂Cl₂:MeOH (10:1) to afford tert-butyl 4-(5-(2-chloropyrimidin-4-yl)-4-(2-fluoro-3-(propylsulfonamido)phenyl)thiazol-2-yl)piperidine-1-carboxylate_(1.58 g, 69.85%) as a yellow solid. LCMS: C₂₆H₃₁ClFN₅O₄S₂ requires: 595.1, found: m/z=596.1 [M+H]⁺.

### Step-4: Synthesis of tert-butyl 4-(5-(2-((tert-butoxycarbonyl)amino)pyrimidin-4-yl)-4-(2-fluoro-3-(propylsulfonamido)phenyl)thiazol-2-yl)piperidine-1-carboxylate (5)

To a mixture of tert-butyl 4-(5-(2-chloropyrimidin-4-yl)-4-(2-fluoro-3-(propylsulfonamido)phenyl)thiazol-2-yl)piperidine-1-carboxylate_(1.0 g, 1.677 mmol, 1 equiv) and BocNH₂ (982.57 mg, 8.385 mmol, 5 equiv) in 1,4-dioxane (12 mL) was added BrettPhos Pd G₃ (152.1 mg, 0.168 mmol, 0.1 equiv) at room temperature. The resulting mixture was stirred for 2 h at 80° C. under nitrogen atmosphere. The resulting mixture was concentrated under reduced pressure. The residue was purified by silica gel column chromatography eluted with PE:EtOAc (1:1) to afford tert-butyl 4-(5-(2-((tert-butoxycarbonyl)amino)pyrimidin-4-yl)-4-(2-fluoro-3-(propylsulfonamido)phenyl)thiazol-2-yl)piperidine-1-carboxylate (850 mg, 87.86%) as a white solid. LCMS: C₃₁H₄₁FN₆O₆S₂ requires: 676.3, found: m/z=677.3 [M+H]⁺.

### Step-5: Synthesis of N-{3-[5-(2-aminopyrimidin-4-yl)-2-(piperidin-4-yl)-1,3-thiazol-4-yl]-2-fluorophenyl}propane-1-sulfonamide Hydrochloride

To a mixture of tert-butyl 4-(5-(2-((tert-butoxycarbonyl)amino)pyrimidin-4-yl)-4-(2-fluoro-3-(propylsulfonamido)phenyl)thiazol-2-yl)piperidine-1-carboxylate (1.5 g, 2.263 mmol, 1 equiv) in DCM (3 mL) was added HCl (gas) in 1,4-dioxane (12 mL, 4 M) at room temperature. The resulting mixture was stirred at room temperature overnight. The resulting mixture was concentrated under reduced pressure. The residue was purified by reverse phase flash chromatography with the following conditions: column, C18 silica gel; mobile phase, MeCN in water (0.1% FA), 10% to 50% gradient in 10 min; detector, UV 254 nm to afford N-{3-[5-(2-aminopyrimidin-4-yl)-2-(piperidin-4-yl)-1,3-thiazol-4-yl]-2-fluorophenyl}propane-1-sulfonamide hydrochloride salt (649.6 mg, 54.60%) as a yellow solid. LCMS: C₂₁H₂₅FN₆O₂S₂ requires: 476.3, found: m/z=477.3 [M+H]⁺.

¹H NMR (400 MHz, CD₃OD) δ 8.09 (d, J=6.0 Hz, 1H), 7.68-7.66 (m, 1H), 7.49-7.28 (m, 2H), 6.65-6.63 (m, 1H), 3.60-3.48 (m, 3H), 3.28-3.16 (m, 2H), 3.17-3.05 (m, 2H), 2.49-2.39 (m, 2H), 2.21-2.09 (m, 2H), 1.94-1.78 (m, 2H), 1.14-0.98 (m, 3H).

### Synthesis of N-(3-{1-[4-(azetidin-3-yl)phenyl]-3-(pyridin-4-yl)pyrazol-4-yl}-2-fluorophenyl)propane-1-sulfonamide; Trifluoroacetic Acid Salt

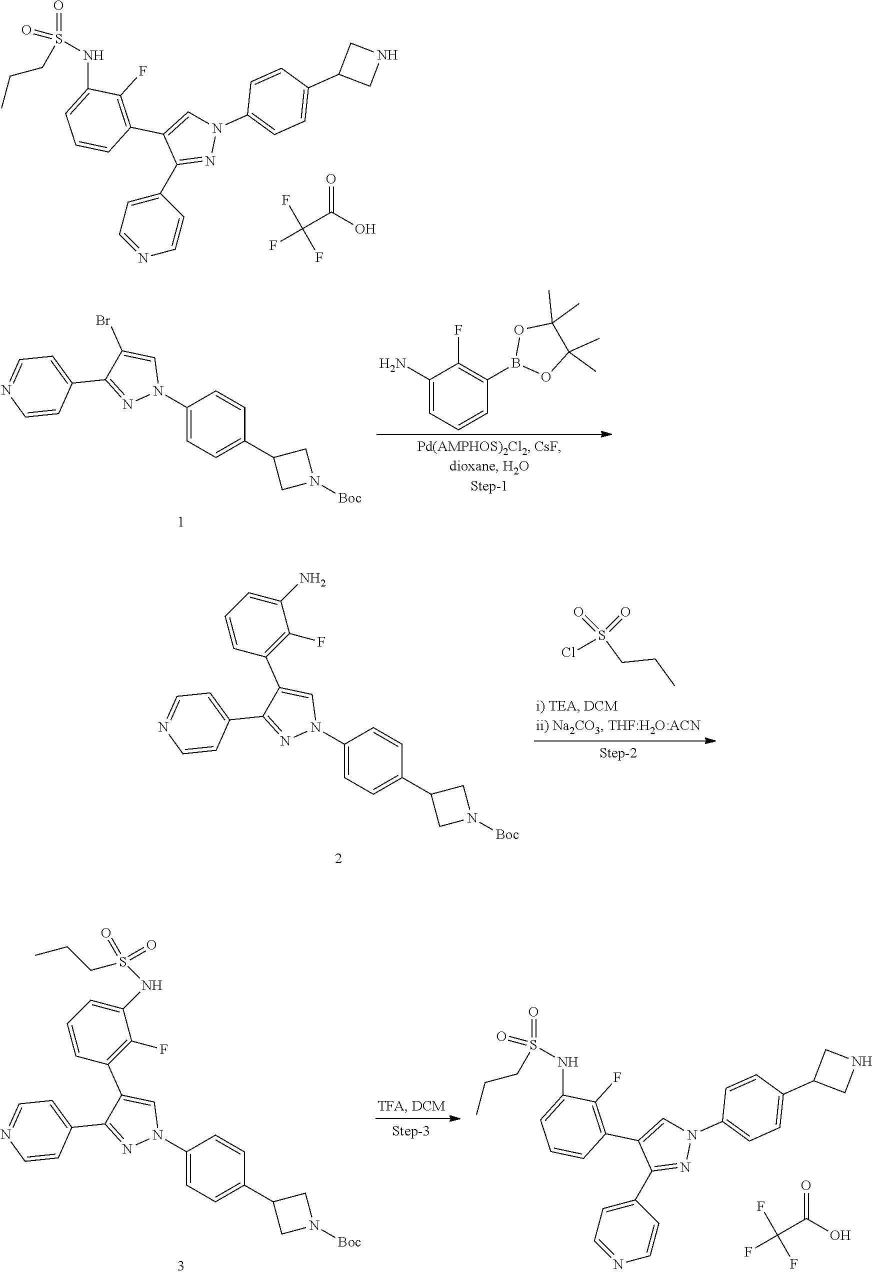

### Step-1: Synthesis of tert-butyl 3-{4-[4-(3-amino-2-fluorophenyl)-3-(pyridin-4-yl)pyrazol-1-yl]phenyl}azetidine-1-carboxylate (2)

To a mixture of tert-butyl 3-{4-[4-bromo-3-(pyridin-4-yl)pyrazol-1-yl]phenyl}azetidine-1-carboxylate (100 mg, 0.22 mmol, 1 equiv) and 2-fluoro-3-(4,4,5,5-tetramethyl-1,3,2-dioxaborolan-2-yl) aniline (78 mg, 0.33 mmol, 1.5 equiv) in dioxane (3 mL) was added CsF (67 mg, 0.44 mmol, 2 equiv) in H₂O (0.3 mL) and Pd(AMPHOS)₂Cl₂ (31 mg, 0.044 mmol, 0.2 equiv). The resulting mixture was stirred at 95° C. for 2 h under nitrogen atmosphere. The resulting mixture was concentrated under vacuum. The residue was purified by silica gel column chromatography eluted with PE:EtOAc (1:10) to afford tert-butyl 3-{4-[4-(3-amino-2-fluorophenyl)-3-(pyridin-4-yl)pyrazol-1-yl]phenyl}azetidine-1-carboxylate (100 mg, 89.09%) as a yellow solid. LCMS: C₂₈H₂₈FN₅O₂ requires: 485.2, found: m/z=486.3 [M+H]⁺.

### Step-2: Synthesis of tert-butyl 3-(4-{4-[2-fluoro-3-(propane-1-sulfonamido)phenyl]-3-(pyridin-4-yl)pyrazol-1-yl}phenyl)azetidine-1-carboxylate (3)

To a mixture of tert-butyl 3-{4-[4-(3-amino-2-fluorophenyl)-3-(pyridin-4-yl)pyrazol-1-yl]phenyl}azetidine-1-carboxylate (60 mg, 0.12 mmol, 1 equiv) and TEA (38 mg, 0.37 mmol, 3 equiv) in DCM (3 mL) was added propane-1-sulfonyl chloride (53 mg, 0.37 mmol, 3 equiv) at 0° C. The resulting mixture was stirred at room temperature for one hour. The resulting mixture was concentrated under vacuum. To the above mixture was added THF (1.5 mL), ACN (1.5 mL), and Na₂CO₃ (196 mg, 1.86 mmol, 15 equiv) in H₂O (1.5 mL). The resulting mixture was stirred at 50° C. overnight. The resulting mixture was concentrated under vacuum. The residue was purified by silica gel column chromatography eluted with PE:EtOAc (1:1) to afford tert-butyl 3-(4-{4-[2-fluoro-3-(propane-1-sulfonamido)phenyl]-3-(pyridin-4-yl)pyrazol-1-yl}phenyl)azetidine-1-carboxylate (50 mg, 64.97%) as a yellow solid. LCMS: C₃₁H₃₄FN₅O₄S requires: 591.2, found: m/z=592.3 [M+H]⁺.

### Step-3: Synthesis of N-(3-{1-[4-(azetidin-3-yl)phenyl]-3-(pyridin-4-yl)pyrazol-4-yl}-2-fluorophenyl)propane-1-sulfonamide; trifluoroacetic acid

To a mixture of tert-butyl 3-(4-{4-[2-fluoro-3-(propane-1-sulfonamido)phenyl]-3-(pyridin-4-yl)pyrazol-1-yl}phenyl)azetidine-1-carboxylate (45 mg, 0.076 mmol, 1 equiv) in DCM (4 mL) was added TFA (1 mL) at 0° C. The resulting mixture was stirred at 0° C. for one hour. The resulting mixture was concentrated under vacuum. The residue was purified by reverse phase flash chromatography eluted with ACN:H₂O (1:1) to afford N-(3-{1-[4-(azetidin-3-yl)phenyl]-3-(pyridin-4-yl)pyrazol-4-yl}-2-fluorophenyl)propane-1-sulfonamide; trifluoroacetic acid salt (18 mg, 37.24%) as a white solid. LCMS: C₂₆H₂₆FN₅O₂S requires: 491.2, found: m/z=492.2 [M+H]⁺. ¹H NMR (400 MHz, CD₃OD) δ 8.66-8.41 (m, 3H), 7.95 (d, J=8.4 Hz, 2H), 7.72-7.46 (m, 5H), 7.37-7.17 (m, 2H), 4.27-4.16 (m, 1H), 4.16-4.08 (m, 2H), 4.07-3.94 (m, 2H), 3.14-2.92 (m, 2H), 1.94-1.64 (m, 2H), 1.12-0.80 (m, 3H).

### Synthesis of N-{3-[5-(2-aminopyrimidin-4-yl)-2-[1-(piperidine-4-carbonyl)piperidin-4-yl]-1,3-thiazol-4-yl]-2-fluorophenyl}propane-1-sulfonamide Hydrochloride Salt

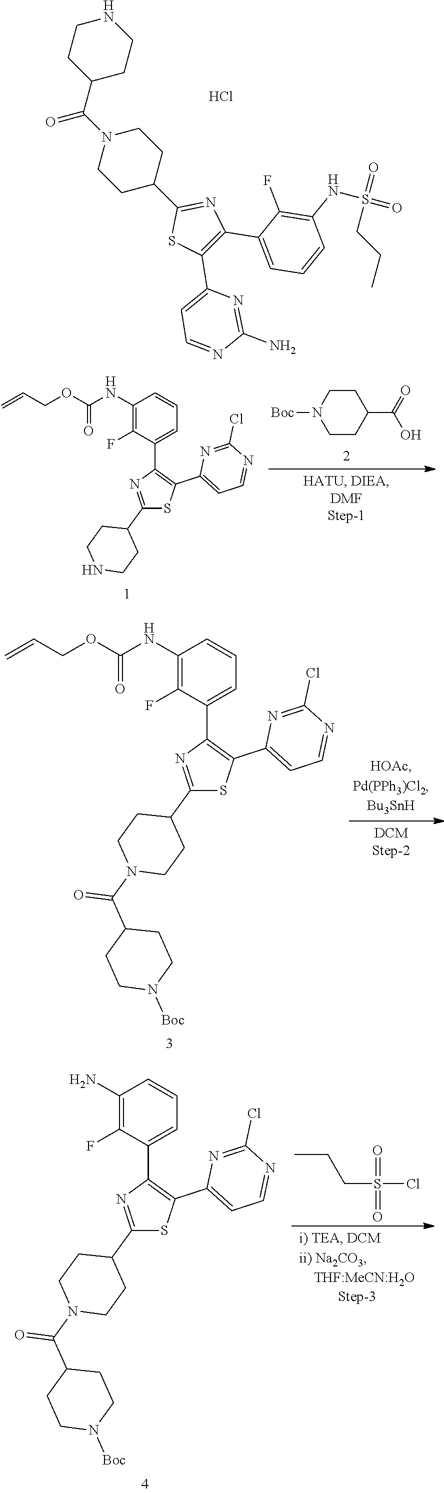

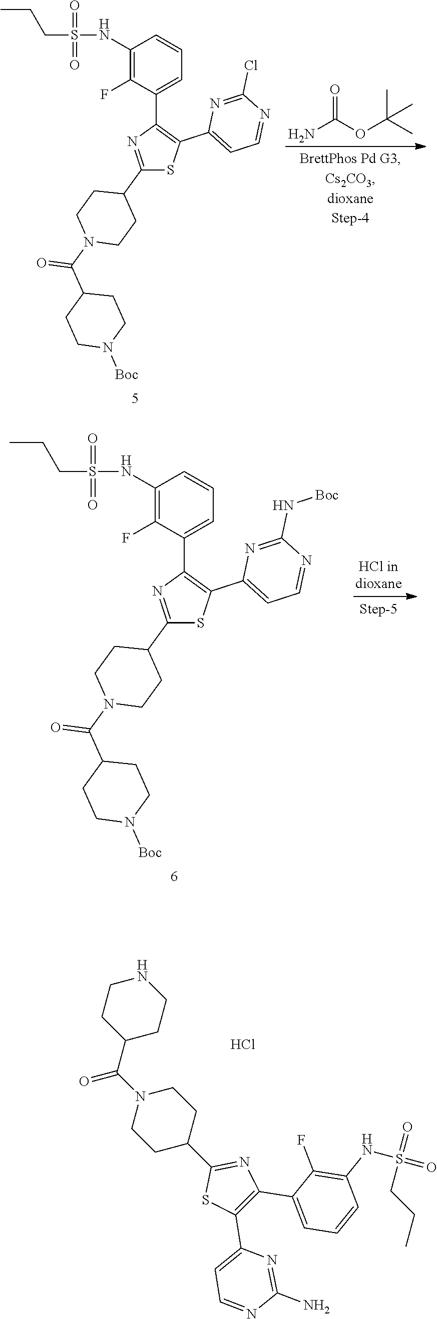

### Step-1: Synthesis of tert-butyl 4-{4-[5-(2-chloropyrimidin-4-yl)-4-(2-fluoro-3-{[(prop-2-en-1-yloxy)carbonyl]amino}phenyl)-1,3-thiazol-2-yl]piperidine-1-carbonyl}piperidine-1-carboxylate (3)

To a mixture of prop-2-en-1-yl N-{3-[5-(2-chloropyrimidin-4-yl)-2-(piperidin-4-yl)-1,3-thiazol-4-yl]-2-fluorophenyl}carbamate (2 g, 4.22 mmol, 1 equiv) and 1-(tert-butoxycarbonyl)piperidine-4-carboxylic acid (1.45 g, 6.33 mmol, 1.5 equiv) in DMF (20 mL) was added HATU (2.41 g, 6.33 mmol, 1.5 equiv) and DIEA (2.73 g, 21.10 mmol, 5 equiv) at room temperature. The resulting mixture was stirred for 2 h at room temperature. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with CH₂Cl₂:MeOH (10:1) to afford tert-butyl 4-{4-[5-(2-chloropyrimidin-4-yl)-4-(2-fluoro-3-{[(prop-2-en-1-yloxy)carbonyl]amino}phenyl)-1,3-thiazol-2-yl]piperidine-1-carbonyl}piperidine-1-carboxylate (1.4 g, 43.58%) as a brown oil. LCMS: C₃₃H₃₈ClFN₆O₅S requires: 684.2, found: m/z=685.3 [M+H]⁺.

### Step-2: Synthesis of tert-butyl 4-{4-[4-(3-amino-2-fluorophenyl)-5-(2-chloropyrimidin-4-yl)-1,3-thiazol-2-yl]piperidine-1-carbonyl}piperidine-1-carboxylate

To a mixture of tert-butyl 4-{4-[5-(2-chloropyrimidin-4-yl)-4-(2-fluoro-3-{[(prop-2-en-1-yloxy)carbonyl]amino}phenyl)-1,3-thiazol-2-yl]piperidine-1-carbonyl}piperidine-1-carboxylate (1.1 g, 1.60 mmol, 1 equiv) and HOAc (241 mg, 4.01 mmol, 2.5 equiv) in DCM (10 mL) was added Pd(PPh₃)₂Cl₂ (22 mg, 0.032 mmol, 0.02 equiv) and n-Bu₃SnH (747 mg, 2.56 mmol, 1.6 equiv) at room temperature under a nitrogen atmosphere. The resulting mixture was stirred for 30 min at room temperature. The reaction was quenched by the addition of saturated aqueous NaHCO₃ at 0° C. The resulting mixture was extracted with CH₂Cl₂. The combined organic layers were washed with water and dried over anhydrous Na₂SO₄. After filtration, the filtrate was concentrated under reduced pressure. The residue was purified by silica gel column chromatography eluted with PE:EtOAc (5:1) to afford tert-butyl 4-{4-[4-(3-amino-2-fluorophenyl)-5-(2-chloropyrimidin-4-yl)-1,3-thiazol-2-yl]piperidine-1-carbonyl}piperidine-1-carboxylate (700 mg, 63.83%) as a yellow oil. LCMS: C₂₉H₃₄ClFN₆O₃S requires: 600.2, found: m/z=601.3 [M+H]⁺.

### Step-3: Synthesis of tert-butyl 4-(4-(5-(2-chloropyrimidin-4-yl)-4-(2-fluoro-3-(propylsulfonamido)phenyl)thiazol-2-yl)piperidine-1-carbonyl)piperidine-1-carboxylate (5)

To a mixture of tert-butyl 4-{4-[4-(3-amino-2-fluorophenyl)-5-(2-chloropyrimidin-4-yl)-1,3-thiazol-2-yl]piperidine-1-carbonyl}piperidine-1-carboxylate (700 mg, 1.16 mmol, 1 equiv) in DCM (7 mL) was added TEA (353 mg, 3.49 mmol, 3 equiv) and propane-1-sulfonyl chloride (199 mg, 1.39 mmol, 1.2 equiv) at 0° C. The resulting mixture was stirred for 2 h at room temperature. The resulting mixture was concentrated under reduced pressure. The crude product was dissolved in THF (3 mL), H₂O (3 mL), and MeCN (3 mL). Na₂CO₃ (456 mg, 4.30 mmol, 5 equiv) was added at room temperature. The resulting mixture was stirred overnight at 50° C. The resulting mixture was concentrated under vacuum. The residue was extracted with DCM. The combined organic layers were washed with water and dried over anhydrous Na₂SO₄. After filtration, the filtrate was concentrated under reduced pressure to afford tert-butyl 4-(4-(5-(2-chloropyrimidin-4-yl)-4-(2-fluoro-3-(propylsulfonamido)phenyl)thiazol-2-yl)piperidine-1-carbonyl)piperidine-1-carboxylate (800 mg, crude) as a brown solid. LCMS: C₃₂H₄₀ClFN₆O₅S₂ requires: 706.2, found: m/z=707.2 [M+H]⁺.

### Step-4: Synthesis of tert-butyl 4-[4-(5-{2-[(tert-butoxycarbonyl)amino]pyrimidin-4-yl}-4-[2-fluoro-3-(propane-1-sulfonamido)phenyl]-1,3-thiazol-2-yl)piperidine-1-carbonyl]piperidine-1-carboxylate (6)

To a mixture of tert-butyl 4-{4-[5-(2-chloropyrimidin-4-yl)-4-[2-fluoro-3-(propane-1-sulfonamido)phenyl]-1,3-thiazol-2-yl]piperidine-1-carbonyl}piperidine-1-carboxylate (1 g, 1.41 mmol, 1 equiv) and tert-butyl carbamate (828 mg, 7.07 mmol, 5 equiv) in dioxane (10 mL) was added BrettPhos Pd G3 (128 mg, 0.14 mmol, 0.1 equiv) and Cs₂CO₃ (921 mg, 2.82 mmol, 2 equiv) at room temperature under a nitrogen atmosphere. The resulting mixture was stirred for 2 h at 80° C. The resulting mixture was then extracted with EtOAc. The combined organic layers were washed with water and dried over anhydrous Na₂SO₄. After filtration, the filtrate was concentrated under reduced pressure. The residue was purified by silica gel column chromatography eluted with CH₂Cl₂:MeOH (10:1) to afford tert-butyl 4-[4-(5-{2-[(tert-butoxycarbonyl)amino]pyrimidin-4-yl}-4-[2-fluoro-3-(propane-1-sulfonamido)phenyl]-1,3-thiazol-2-yl)piperidine-1-carbonyl]piperidine-1-carboxylate (700 mg, 56.55%) as a yellow solid. LCMS: C₃₇H₅₀FN₇O₇S₂ requires: 787.3, found: m/z=788.3 [M+H]⁺.

### Step-5: Synthesis of N-{3-[5-(2-aminopyrimidin-4-yl)-2-[1-(piperidine-4-carbonyl)piperidin-4-yl]-1,3-thiazol-4-yl]-2-fluorophenyl}propane-1-sulfonamide Hydrochloride

A mixture of tert-butyl 4-[4-(5-{2-[(tert-butoxycarbonyl)amino]pyrimidin-4-yl}-4-[2-fluoro-3-(propane-1-sulfonamido)phenyl]-1,3-thiazol-2-yl)piperidine-1-carbonyl]piperidine-1-carboxylate (700 mg, 0.761 mmol, 1 equiv) in HCl in 1,4-dioxane (10 mL, 4 M) was stirred overnight at room temperature. The resulting mixture was concentrated under reduced pressure to afford N-{3-[5-(2-aminopyrimidin-4-yl)-2-[1-(piperidine-4-carbonyl)piperidin-4-yl]-1,3-thiazol-4-yl]-2-fluorophenyl}propane-1-sulfonamide hydrochloride salt (544.7 mg, crude) as a yellow solid. LCMS: C₂₇H₃₄FN₇O₃S₂ requires: 587.2, found: m/z=588.2 [M+H]⁺.

¹H NMR (400 MHz, CD₃OD) δ 8.09-8.06 (m, 1H), 7.72-7.68 (m, 1H), 7.46-7.40 (m, 1H), 7.38-7.36 (m, 1H), 6.62-6.61 (m, 1H), 4.65-4.62 (m, 1H), 4.30-4.20 (m, 1H), 3.77-3.70 (m, 1H), 3.69-3.61 (m, 2H), 3.69-3.50 (m, 1H), 3.47-3.38 (m, 3H), 3.32-3.12 (m, 5H), 2.92-2.90 (m, 1H), 2.28-2.23 (m, 3H), 1.91-1.84 (m, 3H), 1.82-1.70 (m, 1H), 1.07-1.04 (m, 3H).

### Synthesis of N-{3-[5-(2-aminopyrimidin-4-yl)-2-[1-(piperidin-4-ylmethyl)piperidin-4-yl]-1,3-thiazol-4-yl]-2-fluorophenyl}propane-1-sulfonamide Hydrochloride Salt

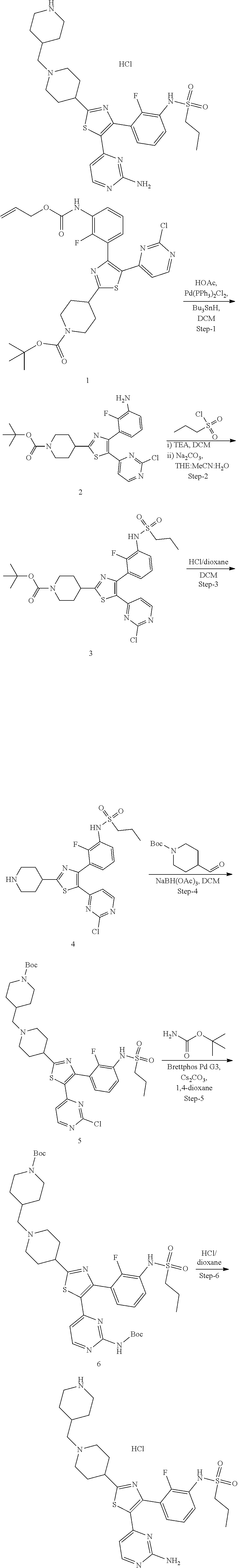

### Step-1: Synthesis of tert-butyl 4-[4-(3-amino-2-fluorophenyl)-5-(2-chloropyrimidin-4-yl)-1,3-thiazol-2-yl]piperidine-1-carboxylate (2)

To a mixture of tert-butyl 4-[5-(2-chloropyrimidin-4-yl)-4-(2-fluoro-3-{[(prop-2-en-1-yloxy)carbonyl]amino}phenyl)-1,3-thiazol-2-yl]piperidine-1-carboxylate (1 g, 1.74 mmol, 1 equiv) and HOAc (261 mg, 4.35 mmol, 2.5 equiv) in DCM (10 mL) was added Pd(PPh₃)₂Cl₂ (24 mg, 0.035 mmol, 0.02 equiv) and n-Bu₃SnH (811 mg, 2.78 mmol, 1.6 equiv) at 0° C. under a nitrogen atmosphere. The resulting mixture was stirred for 30 min at room temperature. The reaction was quenched by saturated aqueous NaHCO₃ at 0° C. The resulting mixture was extracted with CH₂Cl₂. The combined organic layers were washed with water and dried over anhydrous Na₂SO₄. After filtration, the filtrate was concentrated under reduced pressure to afford tert-butyl 4-[4-(3-amino-2-fluorophenyl)-5-(2-chloropyrimidin-4-yl)-1,3-thiazol-2-yl]piperidine-1-carboxylate (1.5 g, crude) as a brown solid. LCMS: C₂₃H₂₅ClFN₅O₂S requires: 489.1, found: m/z=490.1 [M+H]⁺.

### Step-2: Synthesis of tert-butyl 4-(5-(2-chloropyrimidin-4-yl)-4-(2-fluoro-3-(propylsulfonamido)phenyl)thiazol-2-yl)piperidine-1-carboxylate (3)

To a mixture of tert-butyl 4-[4-(3-amino-2-fluorophenyl)-5-(2-chloropyrimidin-4-yl)-1,3-thiazol-2-yl]piperidine-1-carboxylate (1.5 g, 3.06 mmol, 1 equiv) in DCM (15 mL) was added TEA (929 mg, 9.18 mmol, 3 equiv) and propane-1-sulfonyl chloride (436 mg, 3.06 mmol, 1 equiv) at 0° C. The resulting mixture was stirred for one hour at room temperature. The resulting mixture was concentrated under reduced pressure. The crude product was dissolved in THF (5 mL), H₂O (5 mL), and MeCN (5 mL). Na₂CO₃ (1.13 g, 10.68 mmol, 5 equiv) was added at room temperature. The resulting mixture was stirred overnight at 50° C. The resulting mixture was concentrated under vacuum. The residue was extracted with DCM. The combined organic layers were washed with water and dried over anhydrous Na₂SO₄. After filtration, the filtrate was concentrated under reduced pressure. The residue was purified by silica gel column chromatography eluted with DCM:MeOH (10:1) to afford tert-butyl 4-(5-(2-chloropyrimidin-4-yl)-4-(2-fluoro-3-(propylsulfonamido)phenyl)thiazol-2-yl)piperidine-1-carboxylate (2 g, 93.03%) as a brown solid. LCMS: C₂₆H₃₁ClFN₅O₄S₂ requires: 595.2, found: m/z=596.2 [M+H]⁺.

### Step-3: Synthesis of N-{3-[5-(2-chloropyrimidin-4-yl)-2-(piperidin-4-yl)-1,3-thiazol-4-yl]-2-fluorophenyl}propane-1-sulfonamide (4)

A mixture of tert-butyl 4-[5-(2-chloropyrimidin-4-yl)-4-[2-fluoro-3-(propane-1-sulfonamido)phenyl]-1,3-thiazol-2-yl]piperidine-1-carboxylate (1.7 g, 2.85 mmol, 1 equiv) in HCl in 1,4-dioxane (20 mL, 4 M) was stirred for 2 h at room temperature. The resulting mixture was concentrated under reduced pressure to afford N-{3-[5-(2-chloropyrimidin-4-yl)-2-(piperidin-4-yl)-1,3-thiazol-4-yl]-2-fluorophenyl}propane-1-sulfonamide (1.4 g, crude) as a yellow oil. LCMS: C₂₁H₂₃ClFN₅O₂S₂ requires: 495.1, found: m/z=496.2 [M+H]⁺.

### Step-4: Synthesis of tert-butyl 4-({4-[5-(2-chloropyrimidin-4-yl)-4-[2-fluoro-3-(propane-1-sulfonamido)phenyl]-1,3-thiazol-2-yl]piperidin-1-yl}methyl)piperidine-1-carboxylate (5)

To a mixture of N-{3-[5-(2-chloropyrimidin-4-yl)-2-(piperidin-4-yl)-1,3-thiazol-4-yl]-2-fluorophenyl}propane-1-sulfonamide (1.4 g, 2.82 mmol, 1 equiv) in DCM (10 mL) was added tert-butyl 4-formylpiperidine-1-carboxylate (902 mg, 4.23 mmol, 1.5 equiv) and NaOAc (231 mg, 2.82 mmol, 1 equiv) at room temperature under a nitrogen atmosphere. The resulting mixture was stirred for one hour at room temperature. To the above mixture was added NaBH(OAc)₃ (1.2 g, 5.64 mmol, 2 equiv) at 0° C. The resulting mixture was stirred for 2 h at room temperature. The reaction was quenched by the addition of water at 0° C. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with CH₂Cl₂:MeOH (10:1) to afford tert-butyl 4-({4-[5-(2-chloropyrimidin-4-yl)-4-[2-fluoro-3-(propane-1-sulfonamido)phenyl]-1,3-thiazol-2-yl]piperidin-1-yl}methyl)piperidine-1-carboxylate (1.2 g, 55.19%) as a yellow solid. LCMS: C₃₂H₄₂ClFN₆O₄S₂ requires: 692.2, found: m/z=693.3 [M+H]⁺.

### Step-5: Synthesis of tert-butyl 4-{[4-(5-{2-[(tert-butoxycarbonyl)amino]pyrimidin-4-yl}-4-[2-fluoro-3-(propane-1-sulfonamido)phenyl]-1,3-thiazol-2-yl)piperidin-1-yl]methyl}piperidine-1-carboxylate (6)

To a mixture of tert-butyl 4-({4-[5-(2-chloropyrimidin-4-yl)-4-[2-fluoro-3-(propane-1-sulfonamido)phenyl]-1,3-thiazol-2-yl]piperidin-1-yl}methyl)piperidine-1-carboxylate (1.2 g, 1.73 mmol, 1 equiv) and tert-butyl carbamate (1.01 g, 8.65 mmol, 5 equiv) in 1,4-dioxane (10 mL) was added BrettPhos Pd G3 (156 mg, 0.17 mmol, 0.10 equiv) and Cs₂CO₃ (1.13 g, 3.46 mmol, 2 equiv) at room temperature under a nitrogen atmosphere. The resulting mixture was stirred for 2 h at 80° C. The resulting mixture was extracted with EtOAc. The combined organic layers were washed with water and dried over anhydrous Na₂SO₄. After filtration, the filtrate was concentrated under reduced pressure. The crude product was purified by reverse phase flash chromatography with H₂O:MeCN (1:7) to afford tert-butyl 4-{[4-(5-{2-[(tert-butoxycarbonyl)amino]pyrimidin-4-yl}-4-[2-fluoro-3-(propane-1-sulfonamido)phenyl]-1,3-thiazol-2-yl)piperidin-1-yl]methyl}piperidine-1-carboxylate (700 mg, 49.64%) as a yellow solid. LCMS: C₃₇H₅₂FN₇O₆S₂ requires: 773.3, found: m/z=774.3 [M+H]⁺.

### Step-6: Synthesis of N-{3-[5-(2-aminopyrimidin-4-yl)-2-[1-(piperidin-4-ylmethyl)piperidin-4-yl]-1,3-thiazol-4-yl]-2-fluorophenyl}propane-1-sulfonamide Hydrochloride

A mixture of tert-butyl 4-{[4-(5-{2-[(tert-butoxycarbonyl)amino]pyrimidin-4-yl}-4-[2-fluoro-3-(propane-1-sulfonamido)phenyl]-1,3-thiazol-2-yl)piperidin-1-yl]methyl}piperidine-1-carboxylate (700 mg, 1.034 mmol, 1 equiv) in HCl/1,4-dioxane (10 mL, 4 M) was stirred overnight at room temperature. The resulting mixture was concentrated under reduced pressure to afford N-{3-[5-(2-aminopyrimidin-4-yl)-2-[1-(piperidin-4-ylmethyl)piperidin-4-yl]-1,3-thiazol-4-yl]-2-fluorophenyl}propane-1-sulfonamide hydrochloride salt (596.3 mg, crude) as a yellow solid. LCMS: C₂₇H₃₆FN₇O₂S₂ requires: 573.2, found: m/z=574.3 [M+H]⁺.

¹H NMR (400 MHz, CD₃OD) δ 8.15-8.12 (m, 1H), 7.74-7.70 (m, 1H), 7.48-7.40 (m, 1H), 7.38-7.33 (m, 1H), 6.64-6.62 (m, 1H), 3.90-3.83 (m, 2H), 3.77-3.68 (m, 1H), 3.65-3.53 (m, 1H), 3.48-3.40 (m, 2H), 3.30-3.19 (m, 3H), 3.17-3.09 (m, 4H), 2.61-2.42 (m, 5H), 2.20-2.10 (m, 2H), 1.89-1.83 (m, 2H), 1.75-1.61 (m, 2H), 1.07-1.03 (m, 3H).

### Synthesis of N-{3-[2-tert-butyl-5-(2-{[1-(piperidin-4-yl)pyrazol-4-yl]amino}pyrimidin-4-yl)-1,3-thiazol-4-yl]-2-fluorophenyl}propane-1-sulfonamide Hydrochloride Salt

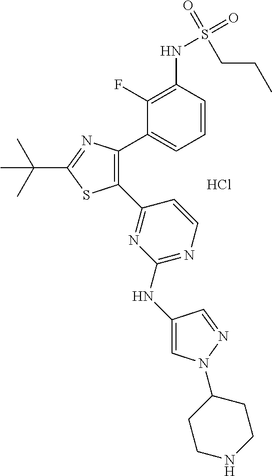 [SMILES: CCCS(=O)(=O)Nc1cccc(-c2nc(C(C)(C)C)sc2-c2ccnc(Nc3cnn(C4CCNCC4)c3)n2)c1F ; Cl]

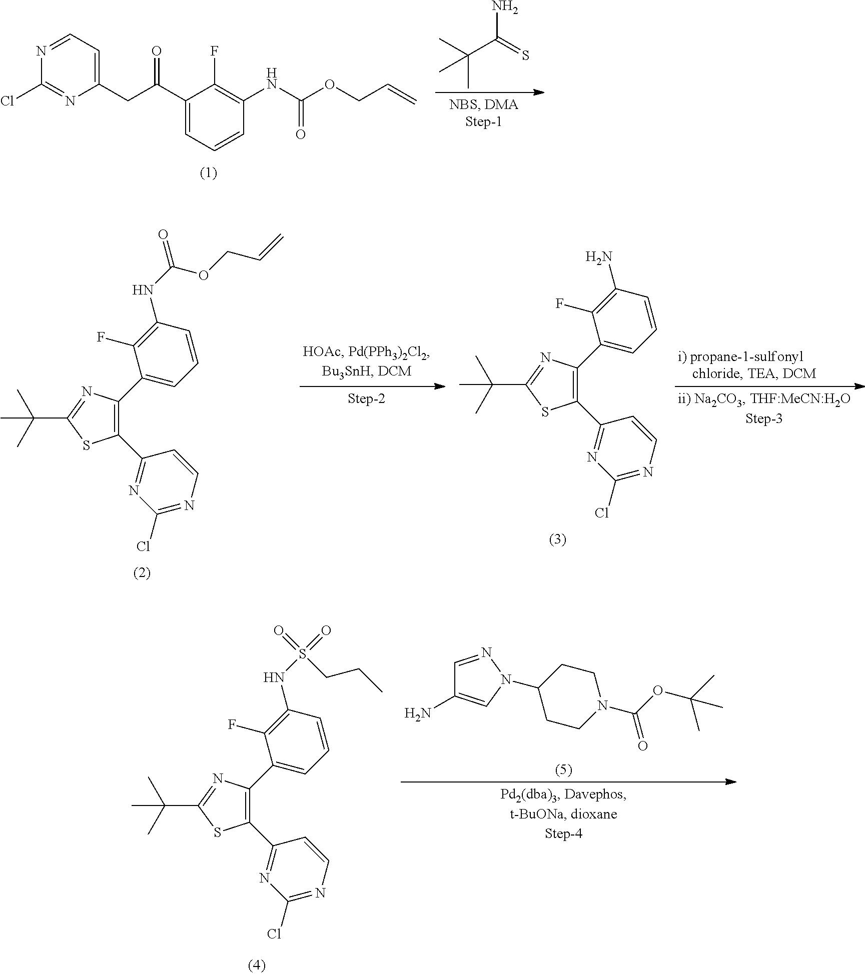

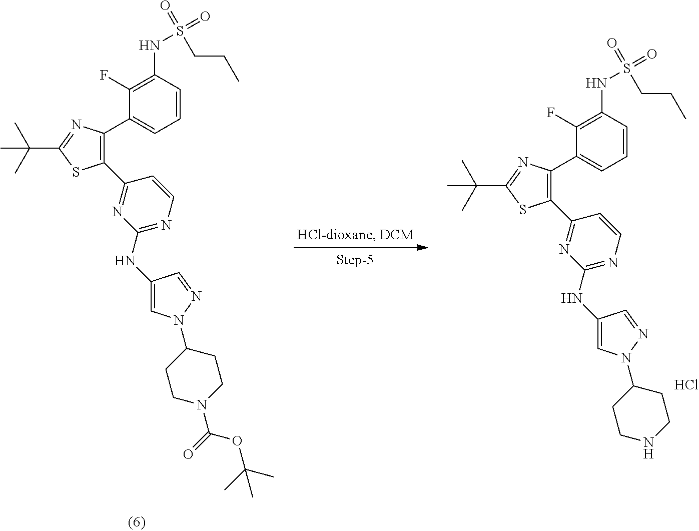 [SMILES: CCCS(=O)(=O)Nc1cccc(-c2nc(C(C)(C)C)sc2-c2ccnc(Nc3cnn(C4CCN(C(=O)OC(C)(C)C)CC4)c3)n2)c1F ; CCCS(=O)(=O)Nc1cccc(-c2nc(C(C)(C)C)sc2-c2ccnc(Nc3cnn(C4CCNCC4)c3)n2)c1F ; Cl]

### Step-1: Synthesis of prop-2-en-1-yl N-{3-[2-tert-butyl-5-(2-chloropyrimidin-4-yl)-1,3-thiazol-4-yl]-2-fluorophenyl}carbamate(2)

To a mixture of 2,2-dimethylpropanethioamide (0.97 g, 8.234 mmol, 1.2 equiv) in DMA (10 mL) was added NBS (1.22 g, 6.862 mmol, 1.0 equiv) at 0° C. The resulting mixture was stirred for 15 min at room temperature. To the above mixture was added prop-2-en-1-yl N-{3-[2-(2-chloropyrimidin-4-yl)acetyl]-2-fluorophenyl}carbamate (2.4 g, 6.862 mmol, 1.0 equiv) at room temperature. The resulting mixture was stirred for additional 2 h at 60° C. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with CH₂Cl₂:MeOH (10:1) to afford prop-2-en-1-yl N-{3-[2-tert-butyl-5-(2-chloropyrimidin-4-yl)-1,3-thiazol-4-yl]-2-fluorophenyl}carbamate (2.6 g, 84.78%) as a yellow solid. LCMS: C₂₁H₂₀ClFN₄O₂S requires: 446.1, found: m/z=447.1 [M+H]⁺.

### Step-2: Synthesis of 3-[2-tert-butyl-5-(2-chloropyrimidin-4-yl)-1,3-thiazol-4-yl]-2-fluoroaniline (3)

To a mixture of prop-2-en-1-yl N-{3-[2-tert-butyl-5-(2-chloropyrimidin-4-yl)-1,3-thiazol-4-yl]-2-fluorophenyl}carbamate (2.5 g, 5.594 mmol, 1 equiv) and HOAc (0.84 g, 13.985 mmol, 2.5 equiv) in DCM (30 mL) was added n-Bu₃SnH (2.61 g, 8.950 mmol, 1.6 equiv) and Pd(PPh₃)₂Cl₂ (78.52 mg, 0.112 mmol, 0.02 equiv) at room temperature under a nitrogen atmosphere. The resulting mixture was stirred for 0.5 h at room temperature under a nitrogen atmosphere. The resulting mixture was concentrated under reduced pressure to afford 3-[2-tert-butyl-5-(2-chloropyrimidin-4-yl)-1,3-thiazol-4-yl]-2-fluoroaniline (2.4 g, crude) as a yellow oil. The crude product was used in the next step directly without further purification. LCMS: C₁₇H₁₆ClFN₄S requires: 362.1, found: m/z=363.1 [M+H]⁺.

### Step-3: Synthesis of N-{3-[2-tert-butyl-5-(2-chloropyrimidin-4-yl)-1,3-thiazol-4-yl]-2-fluorophenyl}propane-1-sulfonamide (4)

To a mixture of 3-[2-tert-butyl-5-(2-chloropyrimidin-4-yl)-1,3-thiazol-4-yl]-2-fluoroaniline (2.4 g, 5.117 mmol, 1 equiv) and TEA (1.55 g, 15.351 mmol, 3.0 equiv) in DCM (30 mL) was added propane-1-sulfonyl chloride (1.46 g, 10.234 mmol, 2.0 equiv) at room temperature. The resulting mixture was stirred for one hour at room temperature. The resulting mixture was concentrated under reduced pressure. The crude product was dissolved in THF (10 mL), CH₃CN (10 mL), and H₂O (10 mL). Na₂CO₃ (5.42 g, 51.170 mmol, 10.0 equiv) was added at room temperature. The resulting mixture was stirred at 50° C. overnight. The resulting mixture was concentrated under reduced pressure. The residue was purified by silica gel column chromatography eluted with CH₂Cl₂:MeOH (10:1) to afford N-{3-[2-tert-butyl-5-(2-chloropyrimidin-4-yl)-1,3-thiazol-4-yl]-2-fluorophenyl}propane-1-sulfonamide (2.0 g, 83.33%) as a yellow solid. LCMS: C₂₀H₂₂ClFN₄O₂S₂ requires: 468.1, found: m/z=469.1 [M+H]⁺.

### Step-4: Synthesis of tert-butyl 4-{4-[(4-{2-tert-butyl-4-[2-fluoro-3-(propane-1-sulfonamido)phenyl]-1,3-thiazol-5-yl}pyrimidin-2-yl)amino]pyrazol-1-yl}piperidine-1-carboxylate (6)

To a mixture of N-{3-[2-tert-butyl-5-(2-chloropyrimidin-4-yl)-1,3-thiazol-4-yl]-2-fluorophenyl}propane-1-sulfonamide (500 mg, 1.099 mmol, 1 equiv) and tert-butyl 4-(4-aminopyrazol-1-yl)piperidine-1-carboxylate (351.26 mg, 1.319 mmol, 1.2 equiv) in dioxane (5 mL) was added DavePhos (129.75 mg, 0.330 mmol, 0.3 equiv), Pd₂(dba)₃ (150.96 mg, 0.165 mmol, 0.15 equiv) and t-BuONa (158.43 mg, 1.648 mmol, 1.5 equiv) at room temperature under a nitrogen atmosphere. The final reaction mixture was irradiated with microwave radiation for 2 h at 120° C. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with CH₂Cl₂:MeOH (10:1) to afford tert-butyl 4-{4-[(4-{2-tert-butyl-4-[2-fluoro-3-(propane-1-sulfonamido)phenyl]-1,3-thiazol-5-yl}pyrimidin-2-yl)amino]pyrazol-1-yl}piperidine-1-carboxylate (600 mg, 78.12%) as a white solid. LCMS: C₃₃H₄₃FN₈O₄S₂ requires: 698.3, found: m/z=699.3 [M+H]⁺.

### Step-5: Synthesis of N-{3-[2-tert-butyl-5-(2-{[1-(piperidin-4-yl)pyrazol-4-yl]amino}pyrimidin-4-yl)-1,3-thiazol-4-yl]-2-fluorophenyl}propane-1-sulfonamide Hydrochloride

To a mixture of tert-butyl 4-{4-[(4-{2-tert-butyl-4-[2-fluoro-3-(propane-1-sulfonamido)phenyl]-1,3-thiazol-5-yl}pyrimidin-2-yl)amino]pyrazol-1-yl}piperidine-1-carboxylate (550 mg, 0.787 mmol, 1 equiv) in DCM (3 mL) was added HCl (gas) in 1,4-dioxane (3 mL, 4N) at room temperature. The resulting mixture was stirred overnight at room temperature. The resulting mixture was concentrated under vacuum. The residue was purified by reverse phase flash chromatography with the following conditions: column, C18 silica gel; mobile phase, MeCN in Water (10 mmol/L NH₄HCO₃), 10% to 50% gradient in 10 min; detector, UV 254 nm to afford N-{3-[2-tert-butyl-5-(2-{[1-(piperidin-4-yl)pyrazol-4-yl]amino}pyrimidin-4-yl)-1,3-thiazol-4-yl]-2-fluorophenyl}propane-1-sulfonamide hydrochloride salt (409.3 mg, 81.88%) as a yellow solid. LCMS: C₂₈H₃₅FN₈O₂S₂ requires: 598.2, found: m/z=599.3 [M+H]⁺. ¹H NMR (400 MHz, CD₃OD) δ 8.38-7.74 (m, 2H), 7.71-7.63 (m, 2H), 7.52 (s, 1H), 7.38-7.32 (m, 1H), 6.77 (s, 1H), 4.57 (s, 1H), 3.64-3.54 (m, 2H), 3.32-3.22 (m, 2H), 3.10 (s, 2H), 2.42-2.26 (m, 4H), 1.92-1.78 (m, 2H), 1.56 (s, 9H), 1.04-0.96 (m, 3H).

### Synthesis of N-(3-{2-tert-butyl-5-[2-(1,2,3,4-tetrahydroisoquinolin-6-ylamino)pyrimidin-4-yl]-1,3-thiazol-4-yl}-2-fluorophenyl)propane-1-sulfonamide Hydrochloride Salt

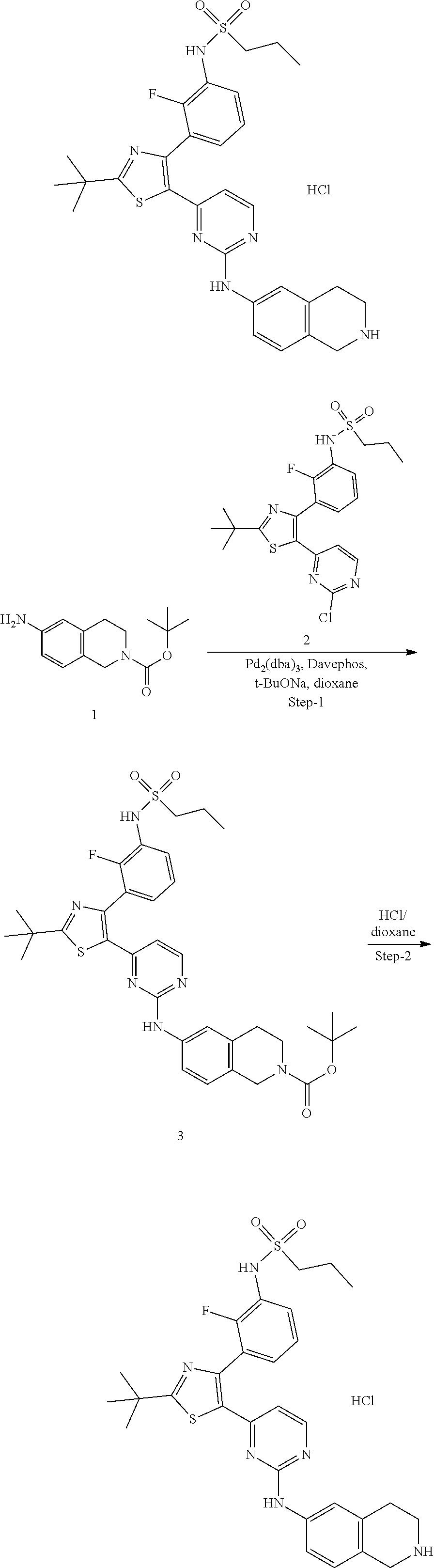

### Step-1: Synthesis of tert-butyl 6-[(4-{2-tert-butyl-4-[2-fluoro-3-(propane-1-sulfonamido)phenyl]-1,3-thiazol-5-yl}pyrimidin-2-yl)amino]-3,4-dihydro-1H-isoquinoline-2-carboxylate (3)

To a mixture of tert-butyl 6-amino-3,4-dihydro-1H-isoquinoline-2-carboxylate (444 mg, 1.79 mmol, 1.2 equiv) and N-{3-[2-tert-butyl-5-(2-chloropyrimidin-4-yl)-1,3-thiazol-4-yl]-2-fluorophenyl}propane-1-sulfonamide (700 mg, 1.49 mmol, 1 equiv) in dioxane (5 mL) was added Pd₂(dba)₃ (205 mg, 0.22 mmol, 0.15 equiv), DavePhos (176 mg, 0.44 mmol, 0.3 equiv), and t-BuONa (215 mg, 2.24 mmol, 1.5 equiv) at room temperature under a nitrogen atmosphere. The final reaction mixture was irradiated with microwave radiation for 2 h at 120° C. under the nitrogen atmosphere. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with PE:EtOAc (5:1) to afford tert-butyl 6-[(4-{2-tert-butyl-4-[2-fluoro-3-(propane-1-sulfonamido)phenyl]-1,3-thiazol-5-yl}pyrimidin-2-yl)amino]-3,4-dihydro-1H-isoquinoline-2-carboxylate (700 mg, 61.99%) as a yellow solid. LCMS: C₃₄H₄₁FN₆O₄S₂ requires: 680.3, found: m/z=681.3 [M+H]⁺.

### Step-2: Synthesis of N-(3-{2-tert-butyl-5-[2-(1,2,3,4-tetrahydroisoquinolin-6-ylamino)pyrimidin-4-yl]-1,3-thiazol-4-yl}-2-fluorophenyl)propane-1-sulfonamide Hydrochloride

A mixture of tert-butyl 6-[(4-{2-tert-butyl-4-[2-fluoro-3-(propane-1-sulfonamido)phenyl]-1,3-thiazol-5-yl}pyrimidin-2-yl)amino]-3,4-dihydro-1H-isoquinoline-2-carboxylate (700 mg, 1.028 mmol, 1 equiv) in HCl/1,4-dioxane (10 mL, 4 M) was stirred overnight at room temperature. The resulting mixture was concentrated under reduced pressure. The residue was purified by reverse phase flash chromatography with MeCN:H₂O (7:1) to afford N-(3-{2-tert-butyl-5-[2-(1,2,3,4-tetrahydroisoquinolin-6-ylamino)pyrimidin-4-yl]-1,3-thiazol-4-yl}-2-fluorophenyl)propane-1-sulfonamide hydrochloride salt (401.2 mg, 66.86%) as a yellow solid. LCMS: C₂₉H₃₃FN₆O₂S₂ requires: 580.2, found: m/z=581.2 [M+H]⁺.

¹H NMR (400 MHz, CD₃OD) δ 8.25-8.20 (m, 1H), 7.66-7.61 (m, 1H), 7.52-7.41 (m, 2H), 7.39-7.34 (m, 2H), 7.25-7.23 (m, 1H), 6.78-6.76 (m, 1H), 4.40 (s, 2H), 3.58-3.55 (m, 2H), 3.32-3.20 (m, 2H), 3.11-3.07 (m, 2H), 1.86-1.80 (m, 3H), 1.54 (s, 9H), 1.50-1.30 (m, 1H), 1.04-1.01 (m, 3H).

### Synthesis of N-{3-[2-tert-butyl-5-(2-{[3-(piperidin-4-yl)phenyl]amino}pyrimidin-4-yl)-1,3-thiazol-4-yl]-2-fluorophenyl}propane-1-sulfonamide Hydrochloride Salt

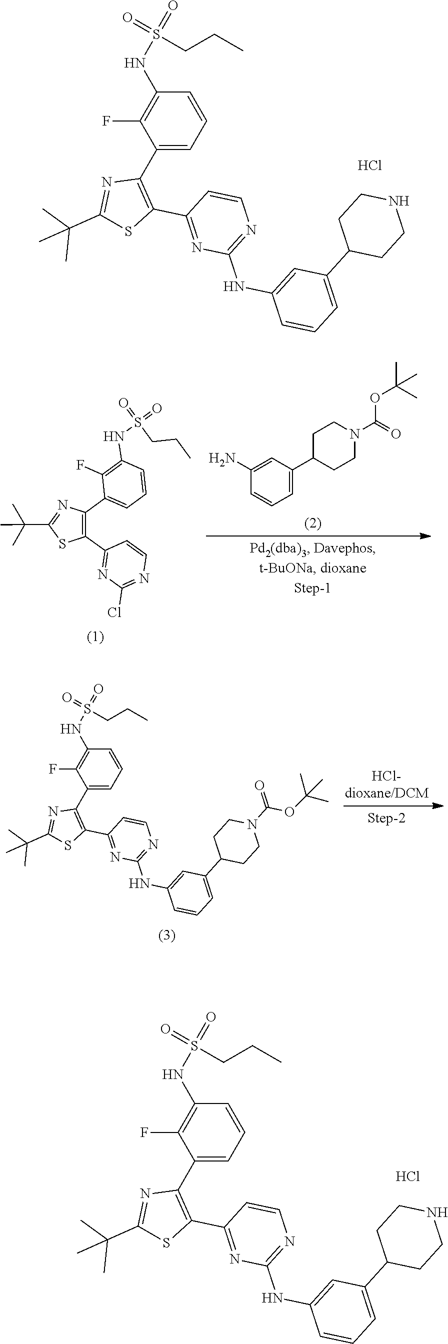

### Step-1: Synthesis of tert-butyl 4-{3-[(4-{2-tert-butyl-4-[2-fluoro-3-(propane-1-sulfonamido)phenyl]-1,3-thiazol-5-yl}pyrimidin-2-yl)amino]phenyl}piperidine-1-carboxylate (3)

To a mixture of N-{3-[2-tert-butyl-5-(2-chloropyrimidin-4-yl)-1,3-thiazol-4-yl]-2-fluorophenyl}propane-1-sulfonamide (500 mg, 1.066 mmol, 1 equiv) and tert-butyl 4-(3-aminophenyl)piperidine-1-carboxylate (353.59 mg, 1.279 mmol, 1.2 equiv) in dioxane (5 mL) was added DavePhos (125.87 mg, 0.320 mmol, 0.3 equiv), Pd₂(dba)₃ (146.44 mg, 0.160 mmol, 0.15 equiv) and t-BuONa (153.69 mg, 1.599 mmol, 1.5 equiv) at room temperature under a nitrogen atmosphere. The final reaction mixture was irradiated with microwave radiation for 2 h at 120° C. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with PE:EtOAc (1:1) to afford tert-butyl 4-{3-[(4-{2-tert-butyl-4-[2-fluoro-3-(propane-1-sulfonamido)phenyl]-1,3-thiazol-5-yl}pyrimidin-2-yl)amino]phenyl}piperidine-1-carboxylate (650 mg, 86.00%) as a yellow solid. LCMS: C₃₆H₄₅FN₆O₄S₂ requires: 708.3, found: m/z=709.3 [M+H]⁺.

### Step-2: Synthesis of N-{3-[2-tert-butyl-5-(2-{[3-(piperidin-4-yl)phenyl]amino}pyrimidin-4-yl)-1,3-thiazol-4-yl]-2-fluorophenyl}propane-1-sulfonamide Hydrochloride

To a mixture of tert-butyl 4-{3-[(4-{2-tert-butyl-4-[2-fluoro-3-(propane-1-sulfonamido)phenyl]-1,3-thiazol-5-yl}pyrimidin-2-yl)amino]phenyl}piperidine-1-carboxylate (550 mg, 0.776 mmol, 1 equiv) in DCM (3 mL) was added HCl (gas) in 1,4-dioxane (3 mL, 4N) at room temperature. The resulting mixture was stirred for 2 h at room temperature. The resulting mixture was concentrated under reduced pressure. The residue was purified by silica gel column chromatography eluted with CH₃CN:H₂O (70:30) to afford N-{3-[2-tert-butyl-5-(2-{[3-(piperidin-4-yl)phenyl]amino}pyrimidin-4-yl)-1,3-thiazol-4-yl]-2-fluorophenyl}propane-1-sulfonamide hydrochloride salt (447 mg, 89.29%) as a yellow solid. LCMS: C₃₁H₃₇FN₆O₂S₂ requires: 608.2, found: m/z=609.3 [M+H]⁺. ¹H NMR (400 MHz, CD₃OD) δ 8.20 (d, J=6.4 Hz, 1H), 7.78-7.68 (m, 1H), 7.52-7.28 (m, 5H), 7.18 (d, J=7.2 Hz, 1H), 6.70 (d, J=6.0 Hz, 1H), 3.58-3.48 (m, 2H), 3.26-3.18 (m, 2H), 3.15-3.05 (m, 2H), 2.99-2.92 (m, 1H), 2.25-2.12 (m, 2H), 2.07-1.92 (m, 2H), 1.89-1.78 (m, 2H), 1.54 (s, 9H), 1.08-1.05 (m, 3H).

### Synthesis of N-{3-[2-tert-butyl-5-(2-{[3-(piperidin-4-yloxy)phenyl]amino}pyrimidin-4-yl)-1,3-thiazol-4-yl]-2-fluorophenyl}propane-1-sulfonamide Hydrochloride Salt

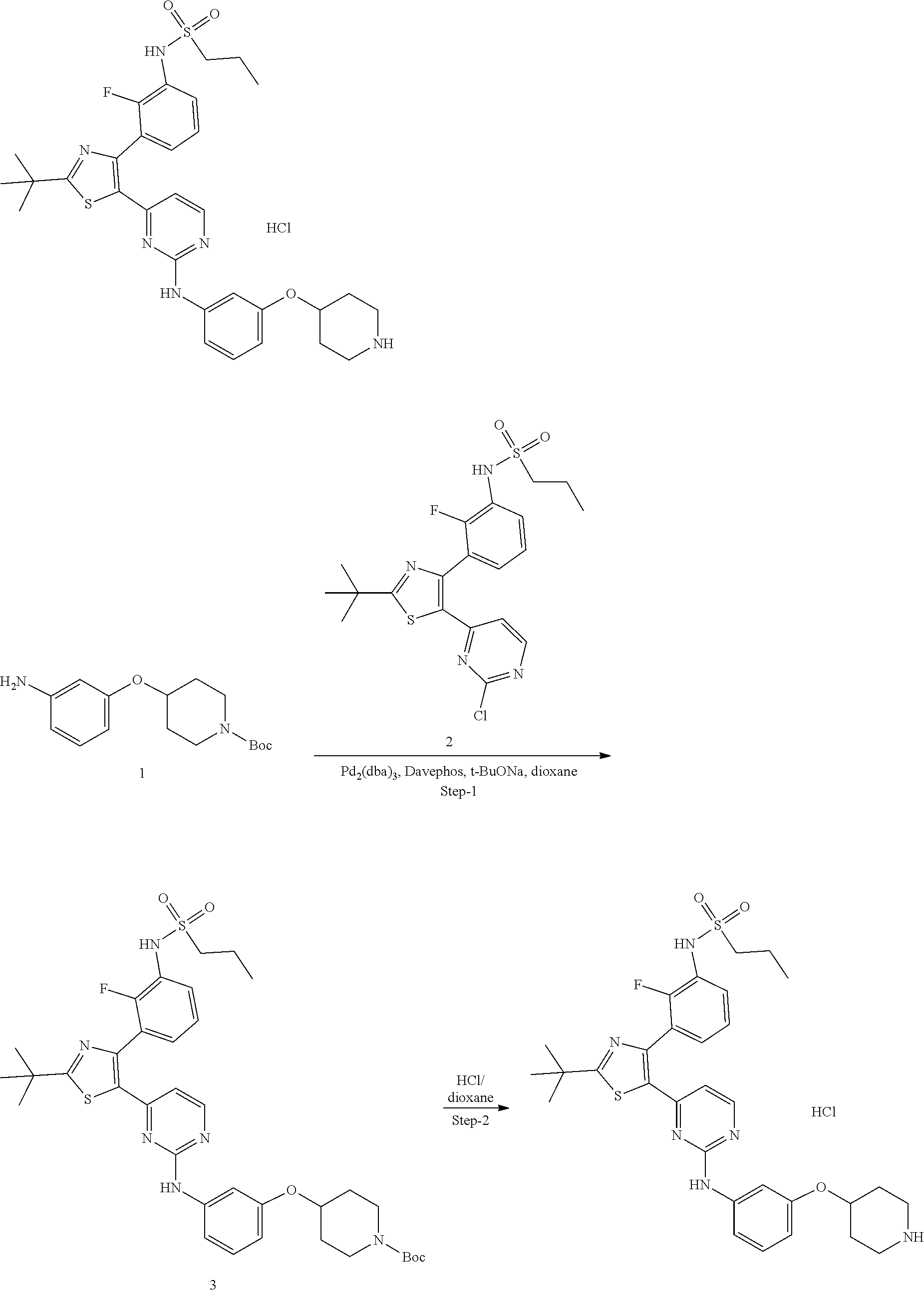

### Step-1: Synthesis of tert-butyl 4-{3-[(4-{2-tert-butyl-4-[2-fluoro-3-(propane-1-sulfonamido)phenyl]-1,3-thiazol-5-yl}pyrimidin-2-yl)amino]phenoxy}piperidine-1-carboxylate (3)

To a mixture of tert-butyl 4-(3-aminophenoxy)piperidine-1-carboxylate (374 mg, 1.27 mmol, 1.2 equiv) and N-{3-[2-tert-butyl-5-(2-chloropyrimidin-4-yl)-1,3-thiazol-4-yl]-2-fluorophenyl}propane-1-sulfonamide (500 mg, 1.06 mmol, 1 equiv) in dioxane (5 mL) was added Pd₂(dba)₃ (146 mg, 0.16 mmol, 0.15 equiv), DavePhos (125 mg, 0.32 mmol, 0.3 equiv) and t-BuONa (153 mg, 1.59 mmol, 1.5 equiv) at room temperature under a nitrogen atmosphere. The final reaction mixture was irradiated with microwave radiation for 2 h at 120° C. under nitrogen atmosphere. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with PE:EtOAc (4:1) to afford tert-butyl 4-{3-[(4-{2-tert-butyl-4-[2-fluoro-3-(propane-1-sulfonamido)phenyl]-1,3-thiazol-5-yl}pyrimidin-2-yl)amino]phenoxy}piperidine-1-carboxylate (500 mg, 58.23%) as a yellow solid. LCMS: C₃₆H₄₅FN₆O₅S₂ requires: 724.2, found: m/z=725.3 [M+H]⁺.

### Step-2: Synthesis of N-{3-[2-tert-butyl-5-(2-{[3-(piperidin-4-yloxy)phenyl]amino}pyrimidin-4-yl)-1,3-thiazol-4-yl]-2-fluorophenyl}propane-1-sulfonamide Hydrochloride

A mixture of tert-butyl 4-{3-[(4-{2-tert-butyl-4-[2-fluoro-3-(propane-1-sulfonamido)phenyl]-1,3-thiazol-5-yl}pyrimidin-2-yl)amino]phenoxy}piperidine-1-carboxylate (500 mg, 0.69 mmol, 1 equiv) in HCl/1,4-dioxane (10 mL, 4 M) was stirred overnight at room temperature. The resulting mixture was concentrated under reduced pressure to afford N-{3-[2-tert-butyl-5-(2-{[3-(piperidin-4-yloxy)phenyl]amino}pyrimidin-4-yl)-1,3-thiazol-4-yl]-2-fluorophenyl}propane-1-sulfonamide hydrochloride salt (414.0 mg, crude) as an orange solid. LCMS: C₃₁H₃₇FN₆O₃S₂ requires: 624.2, found: m/z=625.2 [M+H]⁺.

¹H NMR (400 MHz, CD₃OD) δ 8.15-8.12 (m, 1H), 7.66-7.62 (m, 1H), 7.46-7.41 (m, 1H), 7.40-7.34 (m, 3H), 7.07-7.05 (m, 2H), 6.73-6.70 (m, 1H), 4.86-4.77 (m, 1H), 3.47-3.41 (m, 2H), 3.36-3.26 (m, 2H), 3.09-3.05 (m, 2H), 2.23-2.20 (m, 2H), 2.10-2.04 (m, 2H), 1.97-1.86 (m, 2H), 1.53 (s, 9H), 1.04-1.00 (m, 3H).

### Synthesis of N-{3-[2-tert-butyl-5-(2-{[3-(pyrrolidin-3-yl)phenyl]amino}pyrimidin-4-yl)-1,3-thiazol-4-yl]-2-fluorophenyl}propane-1-sulfonamide Hydrochloride Salt

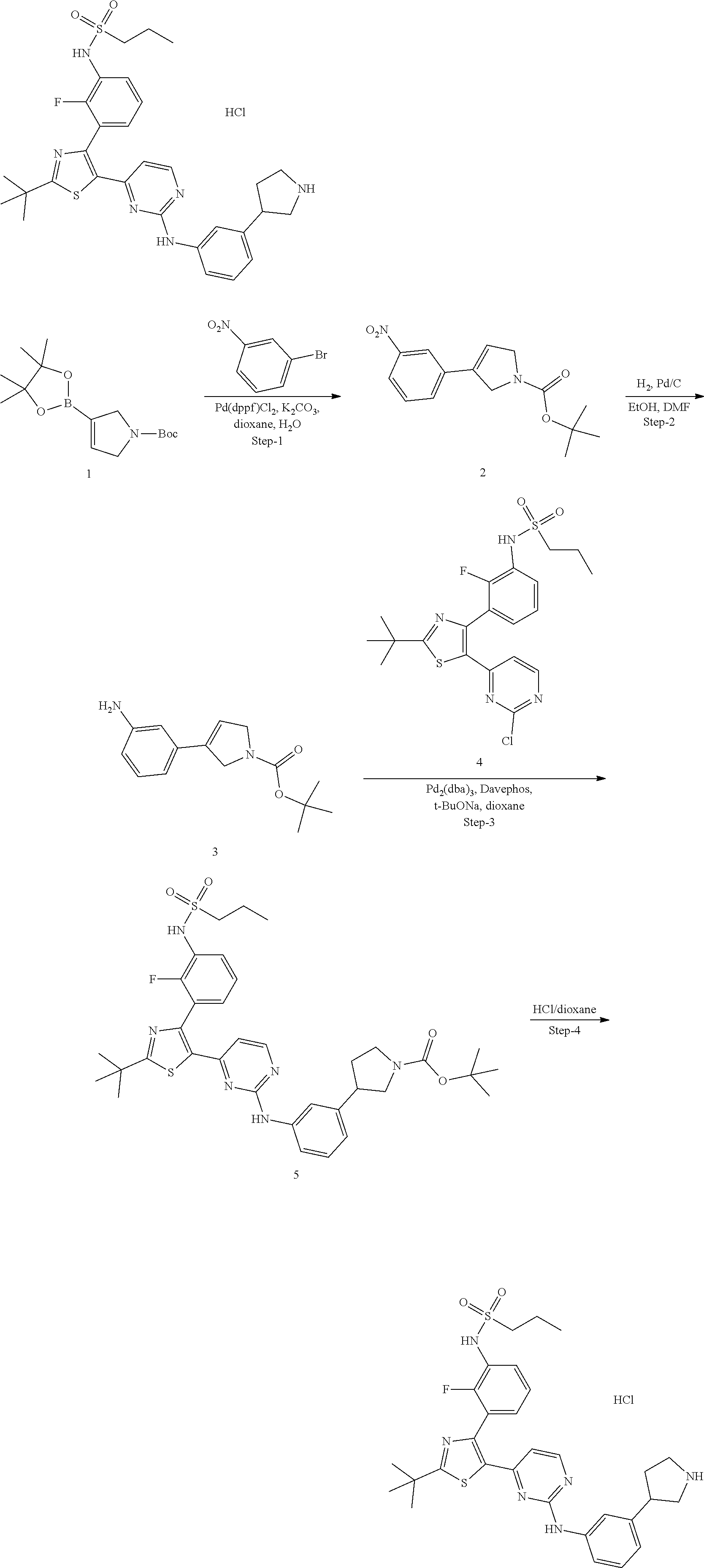

### Step-1: Synthesis of tert-butyl 3-(4-nitrophenyl)-2,5-dihydropyrrole-1-carboxylate (2)

To a mixture of tert-butyl 3-(4,4,5,5-tetramethyl-1,3,2-dioxaborolan-2-yl)-2,5-dihydropyrrole-1-carboxylate (1 g, 3.388 mmol, 1 equiv) and Pd(dppf)Cl₂ (148.73 mg, 0.203 mmol, 0.06 equiv) in dioxane (9 mL) and H₂O (3 mL) was added Cs₂CO₃ (2.21 g, 6.776 mmol, 2 equiv) and 3-bromo-1-nitrobenzene (684.33 mg, 3.388 mmol, 1 equiv) at room temperature under a nitrogen atmosphere. The resulting mixture was stirred overnight at 90° C. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with PE:EtOAc (4:1) to afford tert-butyl 3-(3-nitrophenyl)-2,5-dihydropyrrole-1-carboxylate (700 mg, 67.62%) as a yellow solid.

### Step-2: Synthesis of tert-butyl 3-(3-aminophenyl)pyrrolidine-1-carboxylate

To a mixture of tert-butyl 3-(3-nitrophenyl)-2,5-dihydropyrrole-1-carboxylate (1.4 g, 4.82 mmol, 1 equiv) in DMF (5 mL) and EtOH (5 mL) was added Pd/C (1.4 g) at room temperature. The resulting mixture was stirred overnight at room temperature under a hydrogen atmosphere. The resulting mixture was filtered. The filtrate was concentrated under reduced pressure. The residue was purified by silica gel column chromatography eluted with CH₂Cl₂:MeOH (10:1) to afford tert-butyl 3-(3-aminophenyl)pyrrolidine-1-carboxylate (900 mg, 64.02%) as a yellow solid. LCMS: C₁₅H₂₂N₂O₂ requires: 262.2, found: m/z=263.1 [M+H]⁺.

### Step-3: Synthesis of tert-butyl 3-{3-[(4-{2-tert-butyl-4-[2-fluoro-3-(propane-1-sulfonamido)phenyl]-1,3-thiazol-5-yl}pyrimidin-2-yl)amino]phenyl}pyrrolidine-1-carboxylate (5)

To a mixture of tert-butyl 3-(3-aminophenyl)pyrrolidine-1-carboxylate (268 mg, 1.02 mmol, 1.2 equiv) and N-{3-[2-tert-butyl-5-(2-chloropyrimidin-4-yl)-1,3-thiazol-4-yl]-2-fluorophenyl}propane-1-sulfonamide (400 mg, 0.85 mmol, 1 equiv) in dioxane (5 mL) was added Pd₂(dba)₃ (117 mg, 0.12 mmol, 0.15 equiv), DavePhos (100 mg, 0.25 mmol, 0.3 equiv) and t-BuONa (122 mg, 1.28 mmol, 1.5 equiv) at room temperature under a nitrogen atmosphere. The final reaction mixture was irradiated with microwave radiation for 2 h at 120° C. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with PE:EtOAc (5:1) to afford tert-butyl 3-{3-[(4-{2-tert-butyl-4-[2-fluoro-3-(propane-1-sulfonamido)phenyl]-1,3-thiazol-5-yl}pyrimidin-2-yl)amino]phenyl}pyrrolidine-1-carboxylate (220 mg, 33.41%) as a yellow solid. LCMS: C₃₅H₄₃FN₆O₄S₂ requires: 694.3, found: m/z=695.2 [M+H]⁺.

### Step-4: Synthesis of N-{3-[2-tert-butyl-5-(2-{[3-(pyrrolidin-3-yl)phenyl]amino}pyrimidin-4-yl)-1,3-thiazol-4-yl]-2-fluorophenyl}propane-1-sulfonamide Hydrochloride

A mixture of tert-butyl 3-{3-[(4-{2-tert-butyl-4-[2-fluoro-3-(propane-1-sulfonamido)phenyl]-1,3-thiazol-5-yl}pyrimidin-2-yl)amino]phenyl}pyrrolidine-1-carboxylate (400 mg, 0.576 mmol, 1 equiv) in HCl/1,4-dioxane (5 mL, 4 M) was stirred overnight at room temperature. The resulting mixture was concentrated under reduced pressure to afford N-{3-[2-tert-butyl-5-(2-{[3-(pyrrolidin-3-yl)phenyl]amino}pyrimidin-4-yl)-1,3-thiazol-4-yl]-2-fluorophenyl}propane-1-sulfonamide hydrochloride (313.1 mg, crude) as a yellow solid. LCMS: C₃₀H₃₅FN₆O₂S₂ requires: 594.2, found: m/z=595.2 [M+H]⁺.

¹H NMR (300 MHz, CD₃OD) δ 8.26 (d, J=5.1 Hz, 1H), 7.65-7.62 (m, 2H), 7.56-7.53 (m, 1H), 7.44-7.41 (m, 1H), 7.38-7.26 (m, 2H), 7.00-6.97 (m, 1H), 6.52-6.50 (m, 1H), 3.75-3.71 (m, 1H), 3.59-3.55 (m, 2H), 3.48-3.40 (m, 1H), 3.28-3.21 (m, 1H), 3.06-3.01 (m, 2H), 2.60-2.50 (m, 1H), 2.25-2.06 (m, 1H), 1.85-1.70 (m, 2H), 1.54 (s, 9H), 1.02-0.97 (m, 3H).

### Synthesis of N-{3-[2-tert-butyl-5-(2-{[1-(piperidin-4-ylmethyl)pyrazol-4-yl]amino}pyrimidin-4-yl)-1,3-thiazol-4-yl]-2-fluorophenyl}propane-1-sulfonamide hydrochloride Salt

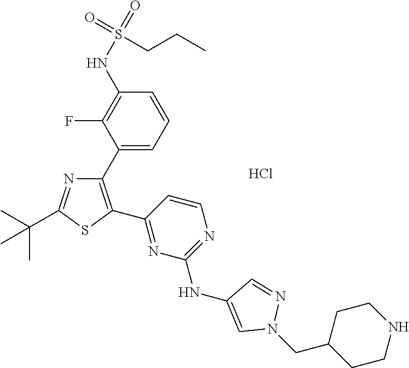 [SMILES: CCCS(=O)(=O)Nc1cccc(-c2nc(C(C)(C)C)sc2-c2ccnc(Nc3cnn(CC4CCNCC4)c3)n2)c1F ; Cl]

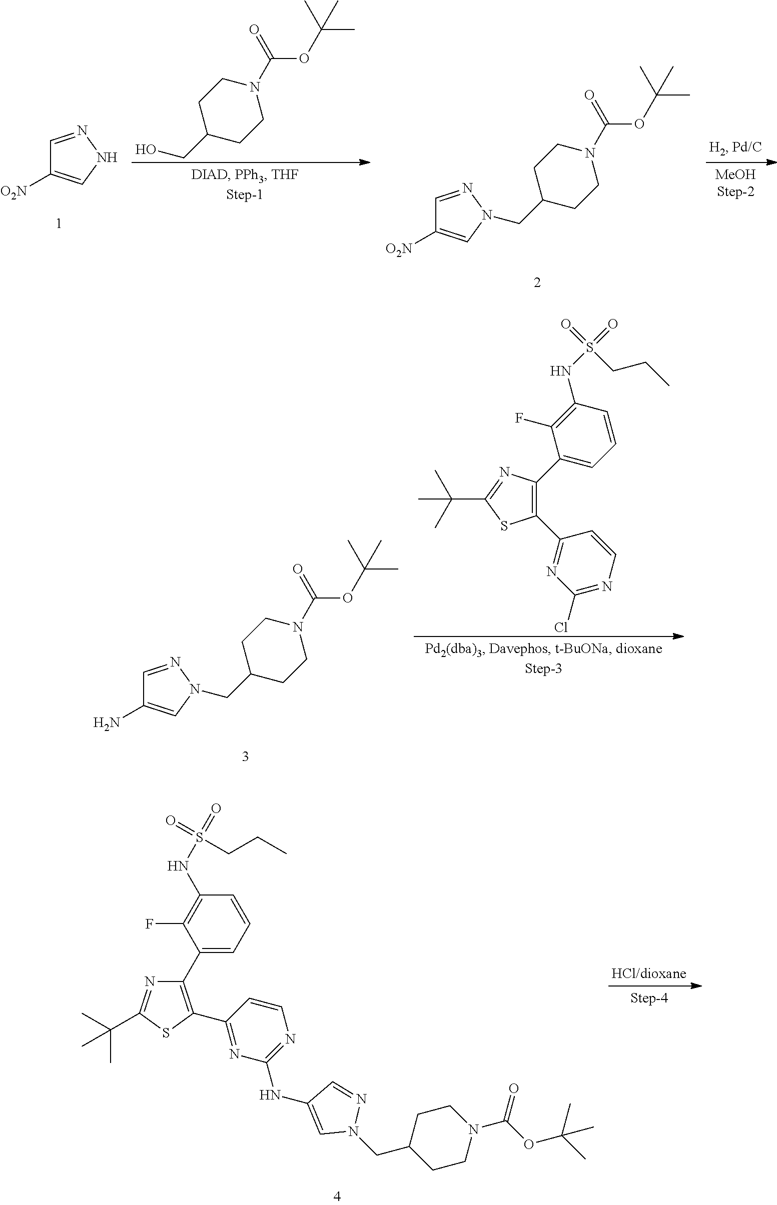 [SMILES: O=[N+]([O-])c1cn[nH]c1 ; CC(C)(C)OC(=O)N1CCC(CO)CC1 ; CC(C)(C)OC(=O)N1CCC(Cn2cc([N+](=O)[O-])cn2)CC1 ; CC(C)(C)OC(=O)N1CCC(Cn2cc(N)cn2)CC1 ; CCCS(=O)(=O)Nc1cccc(-c2nc(C(C)(C)C)sc2-c2ccnc(Cl)n2)c1F ; CCCS(=O)(=O)Nc1cccc(-c2nc(C(C)(C)C)sc2-c2ccnc(Nc3cnn(CC4CCN(C(=O)OC(C)(C)C)CC4)c3)n2)c1F]

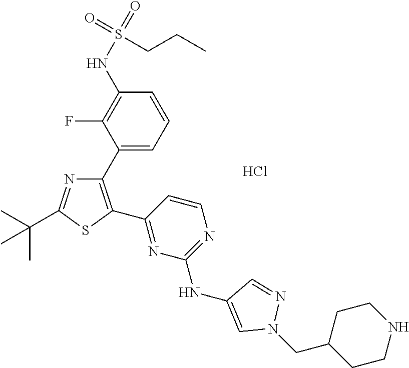 [SMILES: CCCS(=O)(=O)Nc1cccc(-c2nc(C(C)(C)C)sc2-c2ccnc(Nc3cnn(CC4CCNCC4)c3)n2)c1F ; Cl]

### Step-1: Synthesis of tert-butyl 4-[(4-nitropyrazol-1-yl)methyl]piperidine-1-carboxylate (2)

To a mixture of 4-nitropyrazole (2 g, 17.68 mmol, 1 equiv) and tert-butyl 4-(hydroxymethyl)piperidine-1-carboxylate (3.81 g, 17.68 mmol, 1 equiv) in THF (20 mL) was added DIAD (3.58 g, 17.68 mmol, 1 equiv) and PPh₃ (4.64 g, 17.68 mmol, 1 equiv) at room temperature. The resulting mixture was stirred overnight at room temperature. The resulting mixture was concentrated under reduced pressure. The residue was purified by silica gel column chromatography eluted with PE:EtOAc (1:1) to afford tert-butyl 4-[(4-nitropyrazol-1-yl)methyl]piperidine-1-carboxylate (2 g, 32.79%) as a yellow solid. LCMS: C₁₄H₂₂N₄O₄ requires: 310.2, found: m/z=311.2 [M+H]⁺.

### Step-2: Synthesis of tert-butyl 4-[(4-aminopyrazol-1-yl)methyl]piperidine-1-carboxylate (3)

To a mixture of tert-butyl 4-[(4-nitropyrazol-1-yl)methyl]piperidine-1-carboxylate (2 g, 6.44 mmol, 1 equiv) in MeOH (10 mL) was added Pd/C (1.0 g) at room temperature. The resulting mixture was stirred for 1 h at room temperature under a hydrogen atmosphere. The resulting mixture was filtered. The filtrate was concentrated under reduced pressure. The residue was purified by silica gel column chromatography eluted with CH₂Cl₂:MeOH (10:1) to afford tert-butyl 4-[(4-aminopyrazol-1-yl)methyl]piperidine-1-carboxylate (1 g, 49.81%) as a pink solid. LCMS: C₁₄H₂₄N₄O₂ requires: 280.2, found: m/z=281.1 [M+H]⁺.

### Step-3: Synthesis of tert-butyl 4-({4-[(4-{2-tert-butyl-4-[2-fluoro-3-(propane-1-sulfonamido)phenyl]-1,3-thiazol-5-yl}pyrimidin-2-yl)amino]pyrazol-1-yl}methyl)piperidine-1-carboxylate (4)

To a mixture of tert-butyl 4-[(4-aminopyrazol-1-yl)methyl]piperidine-1-carboxylate (358 mg, 1.27 mmol, 1.2 equiv) and N-{3-[2-tert-butyl-5-(2-chloropyrimidin-4-yl)-1,3-thiazol-4-yl]-2-fluorophenyl}propane-1-sulfonamide (500 mg, 1.06 mmol, 1 equiv) in dioxane (5 mL) was added Pd₂(dba)₃ (146 mg, 0.16 mmol, 0.15 equiv), DavePhos (125 mg, 0.32 mmol, 0.3 equiv), and t-BuONa (153 mg, 1.59 mmol, 1.5 equiv) at room temperature under nitrogen atmosphere. The final reaction mixture was irradiated with microwave radiation for 2 h at 120° C. under nitrogen atmosphere. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with PE:EtOAc (6:1) to afford tert-butyl 4-({4-[(4-{2-tert-butyl-4-[2-fluoro-3-(propane-1-sulfonamido)phenyl]-1,3-thiazol-5-yl}pyrimidin-2-yl)amino]pyrazol-1-yl}methyl)piperidine-1-carboxylate (500 mg, 59.21%) as a yellow oil. LCMS: C₃₄H₄₅FN₈O₄S₂ requires: 712.3, found: m/z=713.3 [M+H]⁺.

### Step-4: Synthesis of N-{3-[2-tert-butyl-5-(2-{[1-(piperidin-4-ylmethyl)pyrazol-4-yl]amino}pyrimidin-4-yl)-1,3-thiazol-4-yl]-2-fluorophenyl}propane-1-sulfonamide Hydrochloride

A mixture of tert-butyl 4-({4-[(4-{2-tert-butyl-4-[2-fluoro-3-(propane-1-sulfonamido)phenyl]-1,3-thiazol-5-yl}pyrimidin-2-yl)amino]pyrazol-1-yl}methyl)piperidine-1-carboxylate (500 mg, 0.70 mmol, 1 equiv) in HCl/1,4-dioxane (10 mL, 4 M) was stirred overnight at room temperature. The resulting mixture was concentrated under reduced pressure. The crude product was purified by reverse phase flash chromatography with MeCN:H₂O (9:1) to afford N-{3-[2-tert-butyl-5-(2-{[1-(piperidin-4-ylmethyl)pyrazol-4-yl]amino}pyrimidin-4-yl)-1,3-thiazol-4-yl]-2-fluorophenyl}propane-1-sulfonamide hydrochloride salt (369.1 mg, 85.19%) as an orange solid. LCMS: C₂₉H₃₇FN₈O₂S₂ requires: 612.3, found: m/z=613.3 [M+H]⁺. ¹H NMR (400 MHz, CD₃OD) δ 8.48-8.17 (m, 1H), 7.75-7.64 (m, 3H), 7.60-7.53 (m, 1H), 7.42-7.38 (m, 1H), 6.84-6.57 (m, 1H), 4.26-4.14 (m, 2H), 3.46-3.33 (m, 2H), 3.19-2.98 (m, 4H), 2.41-2.20 (m, 1H), 1.93-1.79 (m, 4H), 1.65-1.56 (m, 11H), 1.09-1.05 (m, 3H).

### Synthesis of N-(2-fluoro-3-{1-[4-(piperidin-4-yl)phenyl]-3-(pyridin-4-yl)pyrazol-4-yl}phenyl)propane-1-sulfonamide Hydrochloride Salt

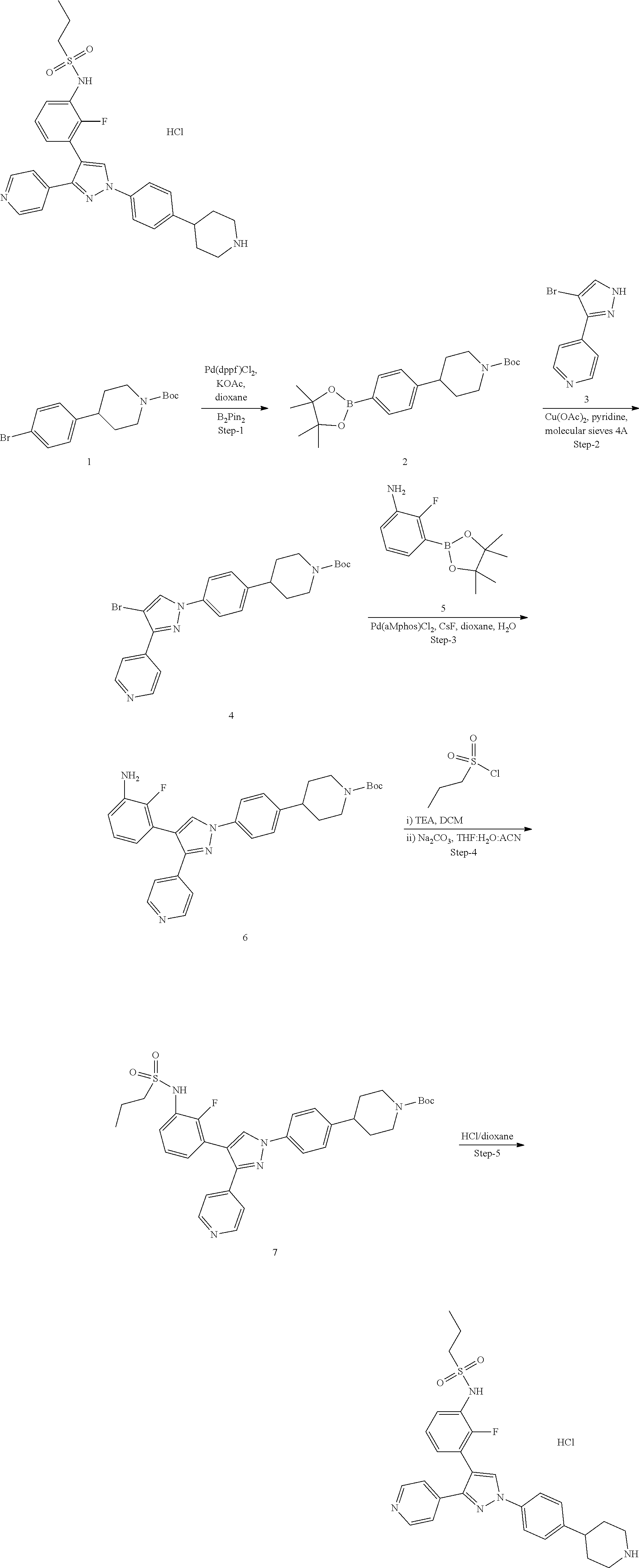

### Step-1: Synthesis of tert-butyl 4-[4-(4,4,5,5-tetramethyl-1,3,2-dioxaborolan-2-yl)phenyl]piperidine-1-carboxylate (2)

To a mixture of tert-butyl 4-(4-bromophenyl)piperidine-1-carboxylate (6 g, 17.634 mmol, 1 equiv) and bis(pinacolato)diboron (13.4 g, 52.902 mmol, 3 equiv) in dioxane (60 mL) was added Pd(dppf)Cl₂·CH₂Cl₂ (1.4 g, 1.763 mmol, 0.1 equiv) and KOAc (5.2 g, 52.902 mmol, 3 equiv) in portions at room temperature under a nitrogen atmosphere. The resulting mixture was stirred at 85° C. overnight under the nitrogen atmosphere. The resulting mixture was concentrated under vacuum. The residue was purified by silica gel column chromatography eluted with PE:EtOAc (82:18) to afford tert-butyl 4-[4-(4,4,5,5-tetramethyl-1,3,2-dioxaborolan-2-yl)phenyl]piperidine-1-carboxylate (4.8 g, 70.28%) as a yellow oil. LCMS: C₂₂H₃₄BNO₄ requires: 387.3, found: m/z=388.2 [M+H]⁺.

### Step-2: Synthesis of tert-butyl 4-{4-[4-bromo-3-(pyridin-4-yl)pyrazol-1-yl]phenyl}piperidine-1-carboxylate (4)

To a mixture of tert-butyl 4-[4-(4,4,5,5-tetramethyl-1,3,2-dioxaborolan-2-yl)phenyl]piperidine-1-carboxylate (2.5 g, 6.454 mmol, 1 equiv) and 4-(4-bromo-1H-pyrazol-3-yl)pyridine (2.2 g, 9.681 mmol, 1.5 equiv) in pyridine (20 mL) was added molecular sieves 4 A (2.2 g) and Cu(OAc)₂ (820.3 mg, 12.908 mmol, 2 equiv) in portions at room temperature under an oxygen atmosphere. The resulting mixture was stirred at 100° C. under the oxygen atmosphere overnight. The resulting mixture was concentrated under vacuum. The residue was purified by silica gel column chromatography eluted with PE:EtOAc (70:30) to afford tert-butyl 4-{4-[4-bromo-3-(pyridin-4-yl)pyrazol-1-yl]phenyl}piperidine-1-carboxylate (2 g, 64.10%) as a light yellow solid. LCMS: C₂₄H₂₇BrN₄O₂ requires: 482.1, found: m/z=483.1 [M+H]⁺.

### Step-3: Synthesis of tert-butyl 4-{4-[4-(3-amino-2-fluorophenyl)-3-(pyridin-4-yl)pyrazol-1-yl]phenyl}piperidine-1-carboxylate (6)

To a mixture of tert-butyl 4-{4-[4-bromo-3-(pyridin-4-yl)pyrazol-1-yl]phenyl}piperidine-1-carboxylate (1.5 g, 3.103 mmol, 1 equiv) and 2-fluoro-3-(4,4,5,5-tetramethyl-1,3,2-dioxaborolan-2-yl) aniline (1.5 g, 6.206 mmol, 2 equiv) in dioxane (15 mL) and H₂O (1.5 mL) was added Pd(AMPHOS)₂Cl₂ (0.44 g, 0.621 mmol, 0.2 equiv) at room temperature under a nitrogen atmosphere. The resulting mixture was stirred at 95° C. under the nitrogen atmosphere for 2 h. The resulting mixture was concentrated under vacuum. The residue was purified by silica gel column chromatography eluted with PE:EtOAc (45:55) to afford tert-butyl 4-{4-[4-(3-amino-2-fluorophenyl)-3-(pyridin-4-yl)pyrazol-1-yl]phenyl}piperidine-1-carboxylate (1.0 g, 62.75%) as a yellow solid. LCMS: C₃₀H₃₂FN₅O₂ requires: 513.3, found: m/z=514.3 [M+H]⁺.

### Step-4: Synthesis of tert-butyl 4-(4-{4-[2-fluoro-3-(propane-1-sulfonamido)phenyl]-3-(pyridin-4-yl)pyrazol-1-yl}phenyl)piperidine-1-carboxylate (7)

To a mixture of tert-butyl 4-{4-[4-(3-amino-2-fluorophenyl)-3-(pyridin-4-yl)pyrazol-1-yl]phenyl}piperidine-1-carboxylate (1.5 g, 2.920 mmol, 1 equiv) and TEA (0.89 g, 8.760 mmol, 3 equiv) in DCM (15 mL) was added propane-1-sulfonyl chloride (0.62 g, 4.380 mmol, 1.5 equiv) dropwise at 0° C. The resulting mixture was stirred at room temperature for one hour. The reaction was quenched with water at 0° C. The resulting mixture was concentrated under vacuum. To the above mixture was added THF (10 mL), ACN (10 mL), and Na₂CO₃ (619.8 mg, 5.847 mmol, 2 equiv) in H₂O (10 mL) at room temperature. The resulting mixture was stirred overnight at 60° C. The resulting mixture was extracted with CH₂Cl₂. The combined organic layers were washed with water and dried over anhydrous Na₂SO₄. After filtration, the filtrate was concentrated under reduced pressure. The residue product was purified by reverse phase flash chromatography with MeCN:H₂O (60:40) to afford tert-butyl 4-(4-{4-[2-fluoro-3-(propane-1-sulfonamido)phenyl]-3-(pyridin-4-yl)pyrazol-1-yl}phenyl)piperidine-1-carboxylate (1.0 g, 55.25%) as a yellow solid. LCMS: C₃₃H₃₈FN₅O₄S requires: 619.3, found: m/z=620.3 [M+H]⁺.

### Step-5: Synthesis of N-(2-fluoro-3-{1-[4-(piperidin-4-yl)phenyl]-3-(pyridin-4-yl)pyrazol-4-yl}phenyl)propane-1-sulfonamide Hydrochloride

A mixture of tert-butyl 4-(4-{4-[2-fluoro-3-(propane-1-sulfonamido)phenyl]-3-(pyridin-4-yl)pyrazol-1-yl}phenyl)piperidine-1-carboxylate (1 g, 1.614 mmol, 1 equiv) and HCl (gas) in 1,4-dioxane (20 mL, 4N) was stirred at room temperature overnight. The resulting mixture was concentrated under vacuum. This resulted in N-(2-fluoro-3-{1-[4-(piperidin-4-yl)phenyl]-3-(pyridin-4-yl)pyrazol-4-yl}phenyl)propane-1-sulfonamide hydrochloride (576.8 mg, crude) as a yellow solid. LCMS: C₂₈H₃₀FN₅O₂S requires: 519.2, found: m/z=520.2 [M+H]⁺.

¹H NMR (400 MHz, CD₃OD) δ 8.81-8.74 (m, 2H), 8.68 (s, 1H), 8.26-8.21 (m, 2H), 8.03-7.96 (m, 2H), 7.65-7.49 (m, 3H), 7.45-7.32 (m, 2H), 3.61-3.52 (m, 2H), 3.26-3.12 (m, 4H), 3.11-3.01 (m, 1H), 2.18-2.12 (m, 2H), 2.09-1.95 (m, 2H), 1.93-1.84 (m, 2H), 1.07-1.03 (m, 3H).

### Synthesis of N-(2-fluoro-3-{1-[4-(piperazin-1-yl)phenyl]-3-(pyridin-4-yl)pyrazol-4-yl}phenyl)propane-1-sulfonamide

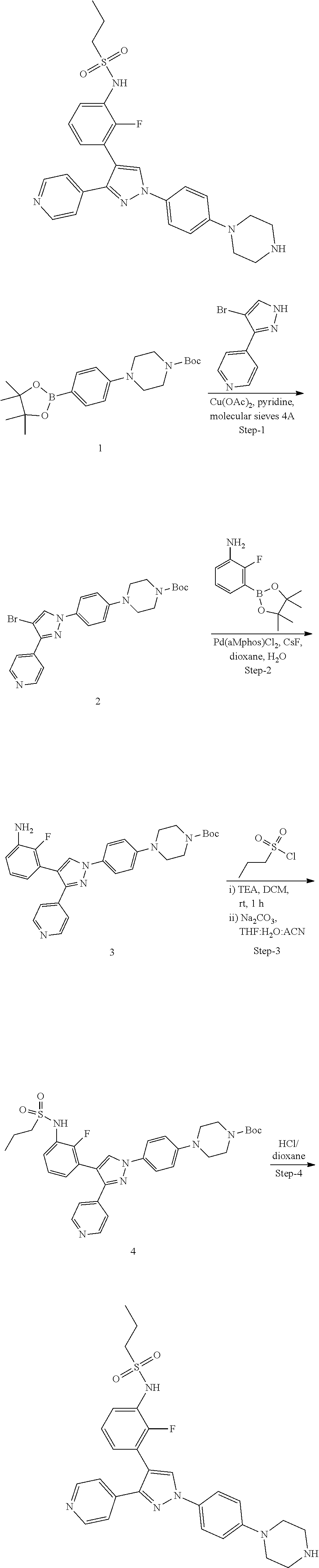

### Step-1: Synthesis of tert-butyl 4-{4-[4-bromo-3-(pyridin-4-yl)pyrazol-1-yl]phenyl}piperazine-1-carboxylate (2)

To a mixture of tert-butyl 4-[4-(4,4,5,5-tetramethyl-1,3,2-dioxaborolan-2-yl)phenyl]piperazine-1-carboxylate (1 g, 2.57 mmol, 1 equiv) in pyridine (10 mL) was added 4-(4-bromo-1H-pyrazol-3-yl)pyridine (865 mg, 3.86 mmol, 1.5 equiv), Cu(OAc)₂ (1.87 g, 5.15 mmol, 2 equiv), and molecular sieves (4 A) (865 mg) at room temperature under an oxygen atmosphere. The resulting mixture was stirred overnight at 100° C. under the oxygen atmosphere. The resulting mixture was concentrated under reduced pressure. The residue was purified by silica gel column chromatography eluted with CH₂Cl₂:MeOH (10:1) to afford tert-butyl 4-{4-[4-bromo-3-(pyridin-4-yl)pyrazol-1-yl]phenyl}piperazine-1-carboxylate (500 mg, 32.07%) as a brown oil. MS (ESI) calc'd for (C₂₃H₂₆BrN₅O₂) [M+H]⁺, 484.1; found, 484.1.

### Step-2: Synthesis of tert-butyl 4-{4-[4-(3-amino-2-fluorophenyl)-3-(pyridin-4-yl)pyrazol-1-yl]phenyl}piperazine-1-carboxylate (3)

To a mixture of tert-butyl 4-{4-[4-bromo-3-(pyridin-4-yl)pyrazol-1-yl]phenyl}piperazine-1-carboxylate (2 g, 4.12 mmol, 1 equiv) in dioxane (20 mL) and H₂O (4 mL) was added 2-fluoro-3-(4,4,5,5-tetramethyl-1,3,2-dioxaborolan-2-yl) aniline (1.96 g, 8.25 mmol, 2 equiv), PdAMPHOS (584 mg, 0.86 mmol, 0.2 equiv), and CsF (1.25 g, 8.25 mmol, 2 equiv) at room temperature under a nitrogen atmosphere. The resulting mixture was stirred 2 h at 95° C. The resulting mixture was concentrated under reduced pressure. The residue was purified by silica gel column chromatography eluted with PE:EtOAc (6:1) to afford tert-butyl 4-{4-[4-(3-amino-2-fluorophenyl)-3-(pyridin-4-yl)pyrazol-1-yl]phenyl}piperazine-1-carboxylate (1.8 g, 76.25%) as a yellow solid. MS (ESI) calc'd for (C₂₉H₃₁FN₆O₂) [M+H]⁺, 515.3; found, 515.1.

### Step-3: Synthesis of tert-butyl 4-(4-{4-[2-fluoro-3-(propane-1-sulfonamido)phenyl]-3-(pyridin-4-yl)pyrazol-1-yl}phenyl)piperazine-1-carboxylate (4)

To a mixture of tert-butyl 4-{4-[4-(3-amino-2-fluorophenyl)-3-(pyridin-4-yl)pyrazol-1-yl]phenyl}piperazine-1-carboxylate (1.8 g, 3.49 mmol, 1 equiv) and TEA (1.06 g, 10.49 mmol, 3 equiv) in DCM (20 mL) was added propane-1-sulfonyl chloride (748 mg, 5.24 mmol, 1.5 equiv) at 0° C. The resulting mixture was stirred one hour at room temperature. The resulting mixture was concentrated under reduced pressure. The crude product was dissolved in THF (10 mL), MeCN (10 mL), and H₂O (10 mL). Then Na₂CO₃ (2 g, 19.25 mmol, 10 equiv) was added at room temperature. The resulting mixture was stirred overnight at 50° C. The resulting mixture was extracted with DCM. The combined organic layers were washed with water and dried over anhydrous Na₂SO₄. After filtration, the filtrate was concentrated under reduced pressure. The residue was purified by silica gel column chromatography eluted with CH₂Cl₂:MeOH (10:1) to afford tert-butyl 4-(4-{4-[2-fluoro-3-(propane-1-sulfonamido)phenyl]-3-(pyridin-4-yl)pyrazol-1-yl}phenyl)piperazine-1-carboxylate (1 g, 58.55%) as a yellow solid. MS (ESI) calc'd for (C₃₂H₃₇FN₆O₄S) [M+H]⁺, 621.3; found, 621.2.

### Step-4: Synthesis of N-(2-fluoro-3-{1-[4-(piperazin-1-yl)phenyl]-3-(pyridin-4-yl)pyrazol-4-yl}phenyl)propane-1-sulfonamide

A mixture of tert-butyl 4-(4-{4-[2-fluoro-3-(propane-1-sulfonamido)phenyl]-3-(pyridin-4-yl)pyrazol-1-yl}phenyl)piperazine-1-carboxylate (1 g, 1.61 mmol, 1 equiv) in HCl in 1,4-dioxane (20 mL, 4 M) was stirred overnight at room temperature. The resulting mixture was concentrated under reduced pressure. The residue was purified by reverse phase flash chromatography with H₂O (0.5% NH₄HCO₃):MeCN (7:1) to afford N-(2-fluoro-3-{1-[4-(piperazin-1-yl)phenyl]-3-(pyridin-4-yl)pyrazol-4-yl}phenyl)propane-1-sulfonamide (514.7 mg, 60.69%) as a white solid. MS (ESI) calc'd for (C₂₇H₂₉FN₆O₂S) [M+H]⁺, 521.2; found, 521.2. ¹H NMR (400 MHz, DMSO-d₆) δ 8.66 (s, 1H), 8.54-8.52 (m, 2H), 7.79-7.77 (m, 2H), 7.47-7.42 (m, 3H), 7.30-7.23 (m, 2H), 7.09-7.07 (m, 2H), 6.20 (s, 1H), 3.15-3.12 (m, 4H), 3.01-2.97 (m, 2H), 2.89-2.81 (m, 4H), 1.72-1.63 (m, 2H), 0.95-0.89 (m, 3H).

### Synthesis of N-(3-(2-(1-(2-(2,7-diazaspiro[3.5]nonan-7-yl)acetyl)piperidin-4-yl)-5-(2-aminopyrimidin-4-yl)thiazol-4-yl)-2-fluorophenyl)propane-1-sulfonamide

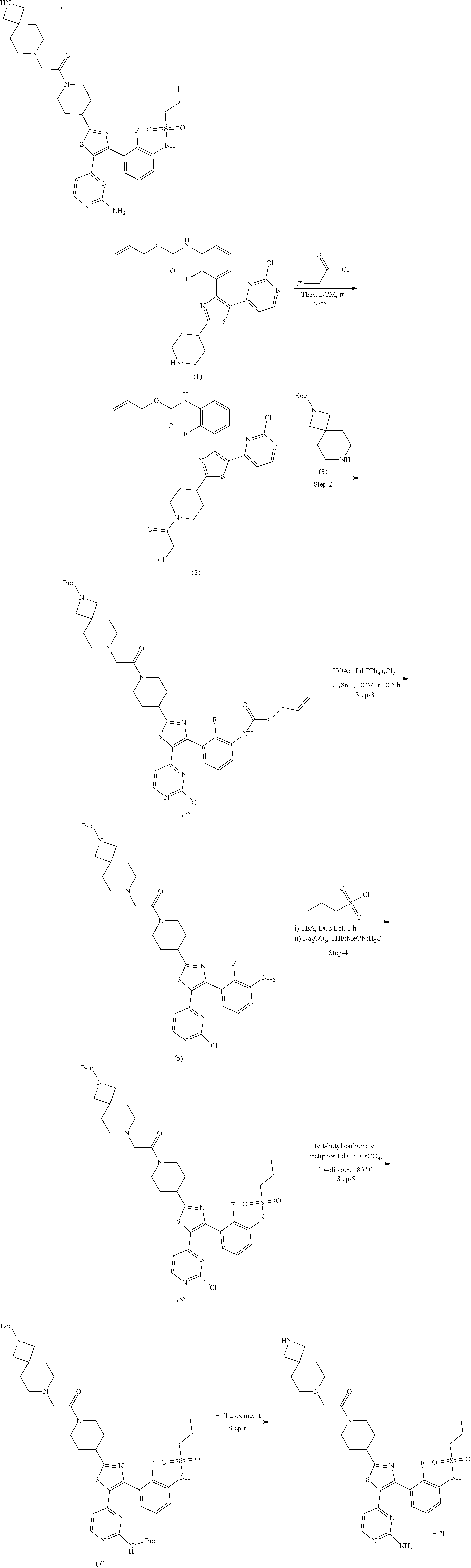

### Step-1: Synthesis of prop-2-en-1-yl N-(3-{2-[1-(2-chloroacetyl)piperidin-4-yl]-5-(2-chloropyrimidin-4-yl)-1,3-thiazol-4-yl}-2-fluorophenyl)carbamate (2)

To a mixture of prop-2-en-1-yl N-{3-[5-(2-chloropyrimidin-4-yl)-2-(piperidin-4-yl)-1,3-thiazol-4-yl]-2-fluorophenyl}carbamate (1000 mg, 2.110 mmol, 1 equiv) and TEA (640.53 mg, 6.330 mmol, 3.0 equiv) in DCM (5 mL) was added chloroacetyl chloride (238.3 mg, 2.110 mmol, 1.0 equiv) at 0° C. The resulting mixture was stirred for 2 h at room temperature. The reaction was quenched with water/ice at room temperature. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with CH₂Cl₂:MeOH (8:1) to afford prop-2-en-1-yl N-(3-{2-[1-(2-chloroacetyl)piperidin-4-yl]-5-(2-chloropyrimidin-4-yl)-1,3-thiazol-4-yl}-2-fluorophenyl)carbamate (800 mg, 68.88%) as a yellow solid. LCMS: C₂₄H₂₂Cl₂FN₅O₃S requires: 549.1, found: m/z=550.1[M+H]⁺.

### Step-2: Synthesis of tert-butyl 7-(2-{4-[5-(2-chloropyrimidin-4-yl)-4-(2-fluoro-3-{[(prop-2-en-1-yloxy)carbonyl]amino}phenyl)-1,3-thiazol-2-yl]piperidin-1-yl}-2-oxoethyl)-2,7-diazaspiro[3.5]nonane-2-carboxylate (4)

To a mixture of prop-2-en-1-yl N-(3-{2-[1-(2-chloroacetyl)piperidin-4-yl]-5-(2-chloropyrimidin-4-yl)-1,3-thiazol-4-yl}-2-fluorophenyl)carbamate (1.5 g, 2.725 mmol, 1 equiv) and tert-butyl 2,7-diazaspiro[3.5]nonane-2-carboxylate (0.31 g, 1.363 mmol, 0.5 equiv) in DMSO (10 mL) was added DIEA (1.06 g, 8.175 mmol, 3 equiv) at room temperature. The resulting mixture was stirred at 80° C. overnight. The resulting mixture was extracted with EtOAc. The combined organic layers were washed with water and dried over anhydrous Na₂SO₄. After filtration, the filtrate was concentrated under reduced pressure. The residue was purified by Prep-TLC (CH₂Cl₂:EtOAc 2:1) to afford tert-butyl 7-(2-{4-[5-(2-chloropyrimidin-4-yl)-4-(2-fluoro-3-{[(prop-2-en-1-yloxy)carbonyl]amino}phenyl)-1,3-thiazol-2-yl]piperidin-1-yl}-2-oxoethyl)-2,7-diazaspiro[3.5]nonane-2-carboxylate (700 mg, 34.70%) as a yellow solid. LCMS: C₃₆H₄₃ClFN₇O₅S requires: 739.3, found: m/z=740.3[M+H]⁺.

### Step-3: Synthesis of tert-butyl 7-(2-{4-[4-(3-amino-2-fluorophenyl)-5-(2-chloropyrimidin-4-yl)-1,3-thiazol-2-yl]piperidin-1-yl}-2-oxoethyl)-2,7-diazaspiro[3.5]nonane-2-carboxylate (5)

To a mixture of tert-butyl 7-(2-{4-[5-(2-chloropyrimidin-4-yl)-4-(2-fluoro-3-{[(prop-2-en-1-yloxy)carbonyl]amino}phenyl)-1,3-thiazol-2-yl]piperidin-1-yl}-2-oxoethyl)-2,7-diazaspiro[3.5]nonane-2-carboxylate (1.2 g, 1.621 mmol, 1 equiv) and Pd(PPh₃)₂Cl₂ (0.02 g, 0.032 mmol, 0.02 equiv) in DCM (15 mL) was added HOAc (0.24 g, 4.053 mmol, 2.5 equiv) at room temperature under a nitrogen atmosphere. To the above mixture was added n-Bu₃SnH (0.75 g, 2.594 mmol, 1.6 equiv) at 0° C. The resulting mixture was stirred for an additional 30 min at room temperature. The resulting mixture was extracted with CH₂Cl₂. The combined organic layers were washed with water and dried over anhydrous Na₂SO₄. After filtration, the filtrate was concentrated under reduced pressure. The residue was purified by silica gel column chromatography eluted with CH₂Cl₂:MeOH (92:8) to afford tert-butyl 7-(2-{4-[4-(3-amino-2-fluorophenyl)-5-(2-chloropyrimidin-4-yl)-1,3-thiazol-2-yl]piperidin-1-yl}-2-oxoethyl)-2,7-diazaspiro[3.5]nonane-2-carboxylate (570 mg, 53.59%) as a yellow solid. LCMS: C₃₂H₃₉ClFN₇O₃S requires: 655.3, found: m/z=656.3[M+H]⁺.

### Step-4: Synthesis of tert-butyl 7-(2-{4-[5-(2-chloropyrimidin-4-yl)-4-[2-fluoro-3-(propane-1-sulfonamido)phenyl]-1,3-thiazol-2-yl]piperidin-1-yl}-2-oxoethyl)-2,7-diazaspiro[3.5]nonane-2-carboxylate (6)

To a mixture of tert-butyl 7-(2-{4-[4-(3-amino-2-fluorophenyl)-5-(2-chloropyrimidin-4-yl)-1,3-thiazol-2-yl]piperidin-1-yl}-2-oxoethyl)-2,7-diazaspiro[3.5]nonane-2-carboxylate (1.1 g, 1.676 mmol, 1 equiv) and TEA (508.88 mg, 5.028 mmol, 3 equiv) in DCM (11 mL) was added propane-1-sulfonyl chloride (358.55 mg, 2.514 mmol, 1.5 equiv) at 0° C. The resulting mixture was stirred for one hour at room temperature. The resulting mixture was concentrated under reduced pressure. To the above mixture was added THF (10 mL), ACN (10 mL), and Na₂CO₃ (0.36 g, 3.352 mmol, 2 equiv) in H₂O (10 mL) at room temperature. The resulting mixture was stirred overnight at 50° C. The resulting mixture was concentrated under reduced pressure. The residue was purified by silica gel column chromatography eluted with CH₂Cl₂:MeOH (10:1) to afford tert-butyl 7-(2-{4-[5-(2-chloropyrimidin-4-yl)-4-[2-fluoro-3-(propane-1-sulfonamido)phenyl]-1,3-thiazol-2-yl]piperidin-1-yl}-2-oxoethyl)-2,7-diazaspiro[3.5]nonane-2-carboxylate (980 mg, 76.69%) as a yellow solid. LCMS: C₃₅H₄₅ClFN₇O₅S₂ requires: 761.3, found: m/z=762.3[M+H]⁺.

### Step-5: Synthesis of tert-butyl 7-{2-[4-(5-{2-[(tert-butoxycarbonyl)amino]pyrimidin-4-yl}-4-[2-fluoro-3-(propane-1-sulfonamido)phenyl]-1,3-thiazol-2-yl)piperidin-1-yl]-2-oxoethyl}-2,7-diazaspiro[3.5]nonane-2-carboxylate (7)

To a mixture of tert-butyl 7-(2-{4-[5-(2-chloropyrimidin-4-yl)-4-[2-fluoro-3-(propane-1-sulfonamido)phenyl]-1,3-thiazol-2-yl]piperidin-1-yl}-2-oxoethyl)-2,7-diazaspiro[3.5]nonane-2-carboxylate (900 mg, 1.181 mmol, 1 equiv) and tert-butyl carbamate (691.49 mg, 5.905 mmol, 5 equiv) in 1,4-dioxane (10 mL) was added BrettPhos Pd G3 (100.87 mg, 0.111 mmol, 0.094 equiv) and Cs₂CO₃ (769.29 mg, 2.362 mmol, 2 equiv) at room temperature under nitrogen atmosphere. The resulting mixture was stirred for 2 h at 80° C. under a nitrogen atmosphere. The resulting mixture was concentrated under vacuum. The residue was purified by silica gel column chromatography eluted with CH₂Cl₂:EtOAc (1:1) to afford tert-butyl 7-{2-[4-(5-{2-[(tert-butoxycarbonyl)amino]pyrimidin-4-yl}-4-[2-fluoro-3-(propane-1-sulfonamido)phenyl]-1,3-thiazol-2-yl)piperidin-1-yl]-2-oxoethyl}-2,7-diazaspiro[3.5]nonane-2-carboxylate (500 mg, 50.24%) as a light yellow solid. LCMS: C₄₀H₅₅FN₈O₇S₂ requires: 842.4, found: m/z=843.4[M+H]⁺.

### Step-6: Synthesis of N-{3-[5-(2-aminopyrimidin-4-yl)-2-[1-(2-{2,7-diazaspiro[3.5]nonan-7-yl}acetyl)piperidin-4-yl]-1,3-thiazol-4-yl]-2-fluorophenyl}propane-1-sulfonamide Hydrochloride

To tert-butyl 7-{2-[4-(5-{2-[(tert-butoxycarbonyl)amino]pyrimidin-4-yl}-4-[2-fluoro-3-(propane-1-sulfonamido)phenyl]-1,3-thiazol-2-yl)piperidin-1-yl]-2-oxoethyl}-2,7-diazaspiro[3.5]nonane-2-carboxylate (500 mg, 0.593 mmol, 1 equiv) was added HCl (gas) in 1,4-dioxane (10 mL, 4N) at room temperature. The resulting mixture was stirred at room temperature overnight. The resulting mixture was concentrated under vacuum. This resulted in N-{3-[5-(2-aminopyrimidin-4-yl)-2-[1-(2-{2,7-diazaspiro[3.5]nonan-7-yl}acetyl)piperidin-4-yl]-1,3-thiazol-4-yl]-2-fluorophenyl}propane-1-sulfonamide hydrochloride salt (501.4 mg, crude) as a yellow solid. LCMS: C₃₀H₃₉FN₈O₃S₂ requires: 642.3, found: m/z=643.2[M+H]⁺.

¹H NMR (400 MHz, CD₃OD) δ 8.10 (d, J=6.8 Hz, 1H), 7.70-7.67 (m, 1H), 7.51-7.44 (m, 1H), 7.43-7.33 (m, 1H), 6.66-6.60 (m, 1H), 4.63-4.57 (m, 1H), 4.50-4.27 (m, 2H), 4.08 (s, 2H), 3.96 (s, 2H), 3.90-3.86 (m, 1H), 3.65-3.58 (m, 2H), 3.55-3.44 (m, 1H), 3.42-3.35 (m, 1H), 3.31-3.20 (m, 2H), 3.19-3.13 (m, 2H), 3.05-3.00 (m, 1H), 2.44-2.35 (m, 2H), 2.33-2.23 (m, 2H), 2.25-2.10 (m, 2H), 2.06-1.94 (m, 1H), 1.90-1.82 (m, 3H), 1.07-1.04 (m, 3H).

### Synthesis of N-{2-fluoro-3-[2-(piperidin-4-yl)-5-(pyrimidin-4-yl)-1,3-thiazol-4-yl]phenyl}propane-1-sulfonamide hydrochloride salt

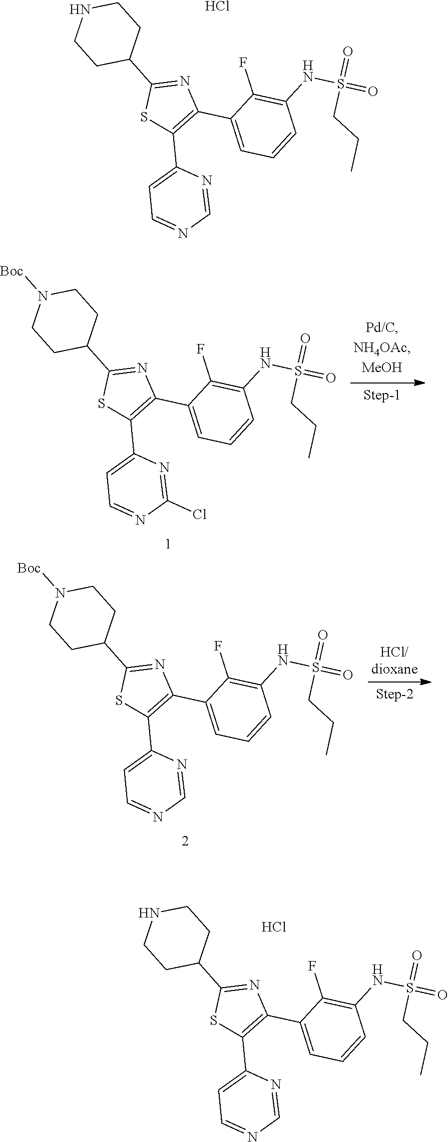 [SMILES: CCCS(=O)(=O)Nc1cccc(-c2nc(C3CCNCC3)sc2-c2ccncn2)c1F ; Cl ; CCCS(=O)(=O)Nc1cccc(-c2nc(C3CCN(C(=O)OC(C)(C)C)CC3)sc2-c2ccnc(Cl)n2)c1F ; CCCS(=O)(=O)Nc1cccc(-c2nc(C3CCN(C(=O)OC(C)(C)C)CC3)sc2-c2ccncn2)c1F ; CCCS(=O)(=O)Nc1cccc(-c2nc(C3CCNCC3)sc2-c2ccncn2)c1F ; Cl]

### Step-1: Synthesis of tert-butyl 4-{4-[2-fluoro-3-(propane-1-sulfonamido)phenyl]-5-(pyrimidin-4-yl)-1,3-thiazol-2-yl}piperidine-1-carboxylate (2)

To a mixture of tert-butyl 4-[5-(2-chloropyrimidin-4-yl)-4-[2-fluoro-3-(propane-1-sulfonamido)phenyl]-1,3-thiazol-2-yl]piperidine-1-carboxylate (700 mg, 1.174 mmol, 1 equiv) and NH₄OAc (905.14 mg, 11.740 mmol, 10 equiv) in MeOH (10 mL) was added Pd/C (700 mg) at room temperature. The resulting mixture was stirred for 30 min at 40° C. The resulting mixture was filtered. The filtrate was concentrated under reduced pressure. The residue was purified by reverse phase chromatography eluted with CH₃CN:H₂O (1:1) to afford tert-butyl 4-{4-[2-fluoro-3-(propane-1-sulfonamido)phenyl]-5-(pyrimidin-4-yl)-1,3-thiazol-2-yl}piperidine-1-carboxylate (450 mg, 68.23%) as a yellow solid. LCMS: C₂₆H₃₂FN₅O₄S₂ requires: 561.2, found: m/z=562.2 [M+H]⁺.

### Step-2: Synthesis of N-{2-fluoro-3-[2-(piperidin-4-yl)-5-(pyrimidin-4-yl)-1,3-thiazol-4-yl]phenyl}propane-1-sulfonamide Hydrochloride

A mixture of tert-butyl 4-{4-[2-fluoro-3-(propane-1-sulfonamido)phenyl]-5-(pyrimidin-4-yl)-1,3-thiazol-2-yl}piperidine-1-carboxylate (450 mg, 0.801 mmol, 1 equiv) in HCl (gas) in 1,4-dioxane (5 mL, 4N) was stirred overnight at room temperature. The resulting mixture was concentrated under reduced pressure. This resulted in N-{2-fluoro-3-[2-(piperidin-4-yl)-5-(pyrimidin-4-yl)-1,3-thiazol-4-yl]phenyl}propane-1-sulfonamide hydrochloride salt (300.1 mg, crude) as a yellow solid. LCMS: C₂₁H₂₄FN₅O₂S₂ requires: 461.1, found: m/z=462.1 [M+H]⁺. ¹H NMR (400 MHz, CD₃OD) δ 9.30-9.28 (m, 1H), 8.76-8.74 (m, 1H), 7.71-7.67 (m, 1H), 7.50-7.38 (m, 3H), 3.60-3.55 (m, 3H), 3.27-3.22 (m, 2H), 3.16-3.12 (m, 2H), 2.48-2.43 (m, 2H), 2.20-2.14 (m, 2H), 1.89-1.83 (m, 2H), 1.06-1.03 (m, 3H).

### Synthesis of N-{2-fluoro-3-[2-(piperidin-4-yl)-5-(pyridin-4-yl)-1,3-thiazol-4-yl]phenyl}propane-1-sulfonamide

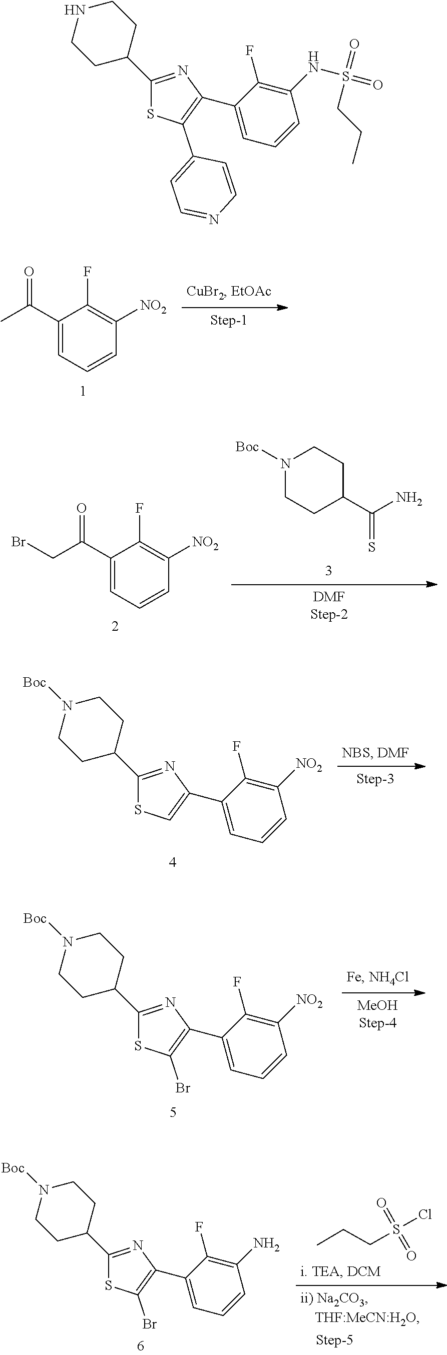

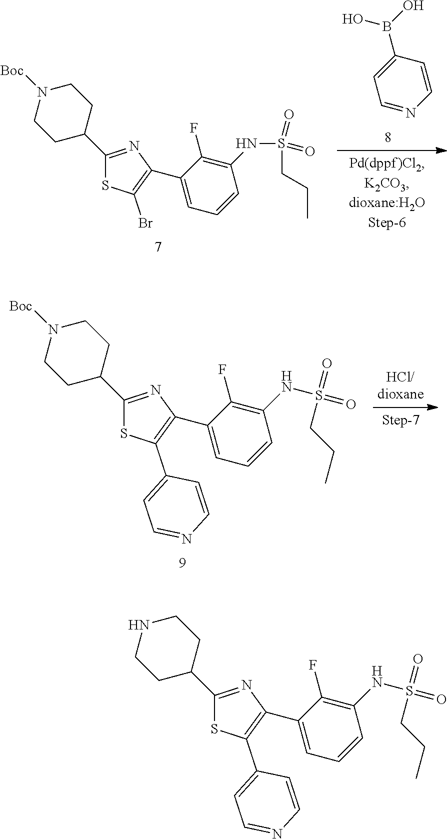 [SMILES: CCCS(=O)(=O)Nc1cccc(-c2nc(C3CCN(C(=O)OC(C)(C)C)CC3)sc2Br)c1F ; OB(O)c1ccncc1 ; CCCS(=O)(=O)Nc1cccc(-c2nc(C3CCN(C(=O)OC(C)(C)C)CC3)sc2-c2ccncc2)c1F ; CCCS(=O)(=O)Nc1cccc(-c2nc(C3CCNCC3)sc2-c2ccncc2)c1F]

### Step-1: Synthesis of 2-bromo-1-(2-fluoro-3-nitrophenyl)ethanone (2)

To a mixture of 1-(2-fluoro-3-nitrophenyl)ethanone (2 g, 10.92 mmol, 1 equiv) in EtOAc (20 mL) was added copper (II) bromide (4.88 g, 21.84 mmol, 2 equiv) at room temperature. The resulting mixture was stirred overnight at 75° C. The precipitated solids were filtered out. The resulting mixture was concentrated under reduced pressure. The residue was purified by silica gel column chromatography eluted with PE:EtOAc (6:1) to afford 2-bromo-1-(2-fluoro-3-nitrophenyl)ethanone (2 g, 61.50%) as a yellow oil. LCMS: C₈H₅BrFNO₃ requires: 260.9, found: m/z=262.0 [M+H]⁺.

### Step-2: Synthesis of tert-butyl 4-[4-(2-fluoro-3-nitrophenyl)-1,3-thiazol-2-yl]piperidine-1-carboxylate (4)

To a mixture of 2-bromo-1-(2-fluoro-3-nitrophenyl)ethanone (2 g, 7.63 mmol, 1 equiv) in DMF (20 mL) was added tert-butyl 4-carbamothioylpiperidine-1-carboxylate (2.05 g, 8.39 mmol, 1.1 equiv) at room temperature. The resulting mixture was stirred for 5 h at room temperature. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with PE:EtOAc (5:1) to afford tert-butyl 4-[4-(2-fluoro-3-nitrophenyl)-1,3-thiazol-2-yl]piperidine-1-carboxylate (2 g, 57.88%) as a yellow oil. LCMS: C₁₉H₂₂FN₃O₄S requires: 407.1, found: m/z=408.1 [M+H]⁺.

### Step-3: Synthesis of tert-butyl 4-[5-bromo-4-(2-fluoro-3-nitrophenyl)-1,3-thiazol-2-yl]piperidine-1-carboxylate (5)

To a mixture of tert-butyl 4-[4-(2-fluoro-3-nitrophenyl)-1,3-thiazol-2-yl]piperidine-1-carboxylate (2 g, 4.90 mmol, 1 equiv) in DMF (20 mL) was added NBS (960 mg, 5.39 mmol, 1.1 equiv) at room temperature. The resulting mixture was stirred overnight at room temperature. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with PE:EtOAc (3:1) to afford tert-butyl 4-[5-bromo-4-(2-fluoro-3-nitrophenyl)-1,3-thiazol-2-yl]piperidine-1-carboxylate (1.2 g, 45.24%) as a yellow oil. LCMS: C₁₉H₂₁BrFN₃O₄S requires: 485.0, found: m/z=486.1 [M+H]⁺.

### Step-4: Synthesis of tert-butyl 4-[4-(3-amino-2-fluorophenyl)-5-bromo-1,3-thiazol-2-yl]piperidine-1-carboxylate (6)

To a mixture of tert-butyl 4-[5-bromo-4-(2-fluoro-3-nitrophenyl)-1,3-thiazol-2-yl]piperidine-1-carboxylate (1 g, 2.05 mmol, 1 equiv) in saturated aqueous NH₄Cl (10 mL) and MeOH (2 mL) was added Fe (114 mg, 2.05 mmol, 5 equiv) at room temperature. The resulting mixture was stirred for 2 h at 70° C. The resulting mixture was concentrated under reduced pressure. The residue was purified by silica gel column chromatography eluted with CH₂Cl₂:MeOH (10:1) to afford tert-butyl 4-[4-(3-amino-2-fluorophenyl)-5-bromo-1,3-thiazol-2-yl]piperidine-1-carboxylate (500 mg, 46.89%) as a yellow oil. LCMS: C₁₉H₂₃BrFN₃O₂S requires: 455.0, found: m/z=456.1 [M+H]⁺.

### Step-5: Synthesis of tert-butyl 4-(5-bromo-4-(2-fluoro-3-(propylsulfonamido)phenyl)thiazol-2-yl)piperidine-1-carboxylate (7)

To a mixture of tert-butyl 4-[4-(3-amino-2-fluorophenyl)-5-bromo-1,3-thiazol-2-yl]piperidine-1-carboxylate (1.2 g, 2.62 mmol, 1 equiv) in DCM (12 mL) was added TEA (798 mg, 7.88 mmol, 3 equiv) and propane-1-sulfonyl chloride (749 mg, 5.25 mmol, 2 equiv) at 0° C. The resulting mixture was stirred for one hour at room temperature. The resulting mixture was concentrated under reduced pressure. The crude product was dissolved in THF (5 mL), H₂O (5 mL), and MeCN (5 mL). Na₂CO₃ (1.13 g, 10.68 mmol, 5 equiv) was added at room temperature. The resulting mixture was stirred overnight at 50° C. The resulting mixture was concentrated under vacuum. The residue was extracted with DCM. The combined organic layers were washed with water and dried over anhydrous Na₂SO₄. After filtration, the filtrate was concentrated under reduced pressure. The residue was purified by silica gel column chromatography eluted with DCM:MeOH (10:1) to afford tert-butyl 4-(5-bromo-4-(2-fluoro-3-(propylsulfonamido)phenyl)thiazol-2-yl)piperidine-1-carboxylate (1 g, 56.88%) as a brown solid. LCMS: C₂₂H₂₉BrFN₃O₄S₂ requires: 561.1, found: m/z=562.1 [M+H]⁺.

### Step-6: Synthesis of tert-butyl 4-{4-[2-fluoro-3-(propane-1-sulfonamido)phenyl]-5-(pyridin-4-yl)-1,3-thiazol-2-yl}piperidine-1-carboxylate (9)

To a mixture of tert-butyl 4-{5-bromo-4-[2-fluoro-3-(propane-1-sulfonamido)phenyl]-1,3-thiazol-2-yl}piperidine-1-carboxylate (1 g, 1.77 mmol, 1 equiv) and pyridin-4-yl boronic acid (218 mg, 1.77 mmol, 1 equiv) in dioxane (10 mL) and H₂O (1 mL) was added Pd(dppf)Cl₂ (130 mg, 0.17 mmol, 0.1 equiv) and K₂CO₃ (491 mg, 3.55 mmol, 2 equiv) at room temperature under a nitrogen atmosphere. The resulting mixture was stirred for 2 h at 80° C. The resulting mixture was concentrated under reduced pressure. The residue was purified by silica gel column chromatography eluted with PE:EtOAc (3:1) to afford tert-butyl 4-{4-[2-fluoro-3-(propane-1-sulfonamido)phenyl]-5-(pyridin-4-yl)-1,3-thiazol-2-yl}piperidine-1-carboxylate (500 mg, 40.13%) as a yellow solid. LCMS: C₂₇H₃₃FN₄O₄S₂ requires: 560.2, found: m/z=561.2 [M+H]⁺.

### Step-7: Synthesis of N-{2-fluoro-3-[2-(piperidin-4-yl)-5-(pyridin-4-yl)-1,3-thiazol-4-yl]phenyl}propane-1-sulfonamide

A mixture of tert-butyl 4-{4-[2-fluoro-3-(propane-1-sulfonamido)phenyl]-5-(pyridin-4-yl)-1,3-thiazol-2-yl}piperidine-1-carboxylate (600 mg, 1.07 mmol, 1 equiv) in HCl/1,4-dioxane (5 mL, 4 M) was stirred overnight at room temperature. The resulting mixture was concentrated under reduced pressure. The crude product (500 mg) was purified by Prep-HPLC with the following conditions Column: XBridge Prep OBD C18 Column, 30*150 mm, 5 m; Mobile Phase A: water (10 mmol/L NH₄HCO₃); Mobile Phase B: ACN; Flow rate: 60 mL/min; Gradient: 20% B to 30% B in 10 min; Wave Length: 254 nm; Rₜ (min): 8.6 to afford N-{2-fluoro-3-[2-(piperidin-4-yl)-5-(pyridin-4-yl)-1,3-thiazol-4-yl]phenyl}propane-1-sulfonamide (324.8 mg, 65.77%) as a white solid. LCMS: C₂₂H₂₅FN₄O₂S₂ requires: 460.1, found: m/z=461.1 [M+H]⁺. ¹H NMR (400 MHz, CD₃OD) δ 8.45-8.44 (m, 2H), 7.61-7.57 (m, 1H), 7.35-7.23 (m, 4H), 3.29-3.19 (m, 3H), 2.95-2.91 (m, 2H), 2.86-2.79 (m, 2H), 2.21-2.18 (m, 2H), 1.86-1.62 (m, 4H), 0.98-0.94 (m, 3H).

### Synthesis of N-{3-[5-(2-aminopyrimidin-4-yl)-2-[4-(piperidin-4-yl)phenyl]-1,3-thiazol-4-yl]-2-fluorophenyl}propane-1-sulfonamide

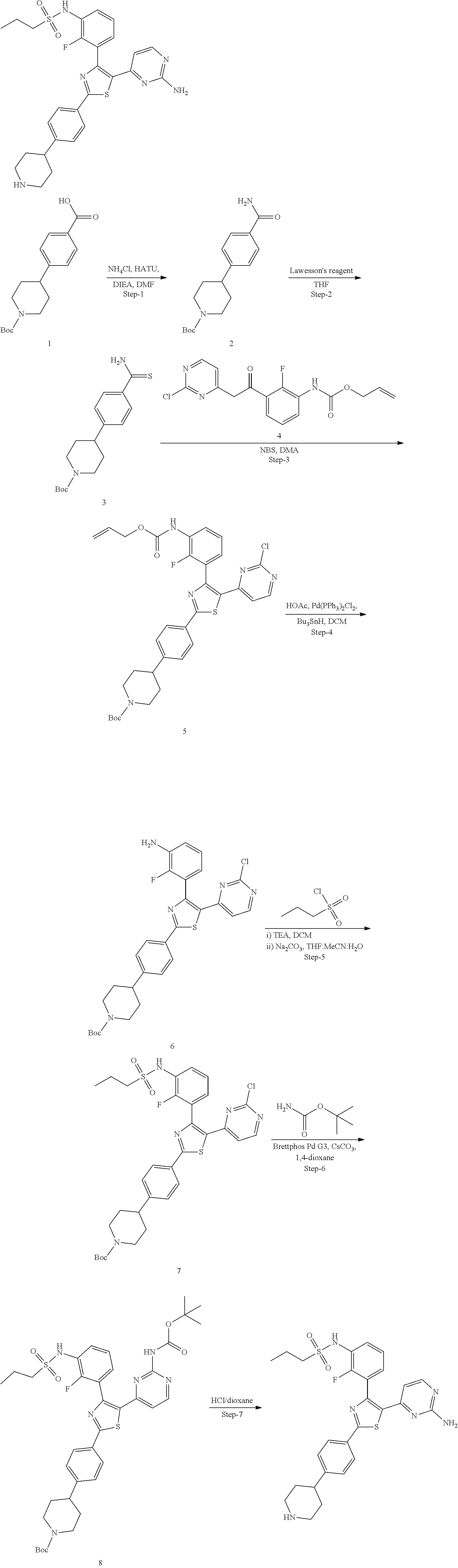

### Step-1: Synthesis of tert-butyl 4-(4-carbamoylphenyl)piperidine-1-carboxylate (2)

To a mixture of 4-[1-(tert-butoxycarbonyl)piperidin-4-yl]benzoic acid (5 g, 16.37 mmol, 1 equiv) and NH₄Cl (4.37 g, 81.86 mmol, 5 equiv) in DMF (50 mL) was added HATU (9.33 g, 24.55 mmol, 1.5 equiv) and DIEA (6.34 g, 49.11 mmol, 3 equiv) at room temperature. The resulting mixture was stirred for one hour at room temperature. The resulting mixture was extracted with EtOAc. The combined organic layers were washed with water and dried over anhydrous Na₂SO₄. After filtration, the filtrate was concentrated under reduced pressure. The product was washed with EtOAc. The precipitated solids were collected by filtration to afford tert-butyl 4-(4-carbamoylphenyl)piperidine-1-carboxylate (4.8 g, 96.31%) as a white solid. LCMS: C₁₇H₂₄N₂O₃ requires: 304.2, found: m/z=305.2 [M+H]⁺.

### Step-2: Synthesis of tert-butyl 4-(4-carbamothioylphenyl)piperidine-1-carboxylate (3)

To a mixture of tert-butyl 4-(4-carbamoylphenyl)piperidine-1-carboxylate (3 g, 9.85 mmol, 1 equiv) in THF (30 mL) were added Lawesson's Reagent (1.99 g, 4.92 mmol, 0.5 equiv) at room temperature. The resulting mixture was stirred overnight at 50° C. The resulting mixture was extracted with EtOAc. The combined organic layers were washed with aqueous NaHCO₃ and dried over anhydrous Na₂SO₄. After filtration, the filtrate was concentrated under reduced pressure. The residue was purified by silica gel column chromatography eluted with CH₂Cl₂:EtOAc (4:1) to afford tert-butyl 4-(4-carbamothioylphenyl)piperidine-1-carboxylate (2.2 g, 69.66%) as a white solid. LCMS: C₁₇H₂₄N₂O₂S requires: 320.1, found: m/z=321.2 [M+H]⁺.

### Step-3: Synthesis of tert-butyl 4-{4-[5-(2-chloropyrimidin-4-yl)-4-(2-fluoro-3-{[(prop-2-en-1-yloxy)carbonyl]amino}phenyl)-1,3-thiazol-2-yl]phenyl}piperidine-1-carboxylate (5)

To a mixture of prop-2-en-1-yl N-{3-[2-(2-chloropyrimidin-4-yl)acetyl]-2-fluorophenyl}carbamate (1.8 g, 5.14 mmol, 1 equiv) in DMA (20 mL) was added tert-butyl 4-(4-carbamothioylphenyl)piperidine-1-carboxylate (1.98 g, 6.17 mmol, 1.2 equiv) and NBS (1.83 g, 10.29 mmol, 2 equiv) at room temperature. The resulting mixture was stirred for 2 h at 60° C. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with PE:EtOAc (5:1) to afford tert-butyl 4-{4-[5-(2-chloropyrimidin-4-yl)-4-(2-fluoro-3-{[(prop-2-en-1-yloxy)carbonyl]amino}phenyl)-1,3-thiazol-2-yl]phenyl}piperidine-1-carboxylate (1.8 g, 48.95%) as a yellow solid. LCMS: C₃₃H₃₃ClFN₅O₄S requires: 649.2, found: m/z=650.2 [M+H]⁺.

### Step-4: Synthesis of tert-butyl 4-{4-[4-(3-amino-2-fluorophenyl)-5-(2-chloropyrimidin-4-yl)-1,3-thiazol-2-yl]phenyl}piperidine-1-carboxylate (6)

To a mixture of tert-butyl 4-{4-[5-(2-chloropyrimidin-4-yl)-4-(2-fluoro-3-{[(prop-2-en-1-yloxy)carbonyl]amino}phenyl)-1,3-thiazol-2-yl]phenyl}piperidine-1-carboxylate (1.8 g, 2.76 mmol, 1 equiv) and HOAc (415 mg, 6.93 mmol, 2.5 equiv) in DCM (20 mL) was added n-Bu₃SnH (1.29 g, 4.43 mmol, 1.6 equiv) and Pd(PPh₃)₂Cl₂ (39 mg, 0.05 mmol, 0.02 equiv) at 0° C. under a nitrogen atmosphere. The resulting mixture was stirred for 30 min at room temperature. The reaction was quenched with saturated aqueous NaHCO₃ at 0° C. The resulting mixture was extracted with CH₂Cl₂. The combined organic layers were washed with water and dried over anhydrous Na₂SO₄. After filtration, the filtrate was concentrated under reduced pressure to afford tert-butyl 4-{4-[4-(3-amino-2-fluorophenyl)-5-(2-chloropyrimidin-4-yl)-1,3-thiazol-2-yl]phenyl}piperidine-1-carboxylate (1.5 g, crude) as a brown solid. LCMS: C₂₉H₂₉ClFN₅O₂S requires: 565.2, found: m/z=566.2 [M+H]⁺.

### Step-5: Synthesis of tert-butyl 4-(4-(5-(2-chloropyrimidin-4-yl)-4-(2-fluoro-3-(propylsulfonamido)phenyl)thiazol-2-yl)phenyl)piperidine-1-carboxylate (7)

To a mixture of tert-butyl 4-{4-[4-(3-amino-2-fluorophenyl)-5-(2-chloropyrimidin-4-yl)-1,3-thiazol-2-yl]phenyl}piperidine-1-carboxylate (1.5 g, 2.65 mmol, 1 equiv) in DCM (15 mL) was added propane-1-sulfonyl chloride (755 mg, 5.30 mmol, 2 equiv) and TEA (804 mg, 7.95 mmol, 3 equiv) at 0° C. The resulting mixture was stirred for one hour at room temperature. The resulting mixture was concentrated under reduced pressure. The crude product was dissolved in THF (5 mL), H₂O (5 mL), and MeCN (5 mL). Na₂CO₃ (1.13 g, 10.68 mmol, 5 equiv) was added at room temperature. The resulting mixture was stirred overnight at 50° C. The resulting mixture was concentrated under vacuum. The residue was extracted with DCM. The combined organic layers were washed with water and dried over anhydrous Na₂SO₄. After filtration, the filtrate was concentrated under reduced pressure. The residue was purified by silica gel column chromatography eluted with DCM:MeOH (10:1) to afford tert-butyl 4-(4-(5-(2-chloropyrimidin-4-yl)-4-(2-fluoro-3-(propylsulfonamido)phenyl)thiazol-2-yl)phenyl)piperidine-1-carboxylate (1.1 g, 73.77%) as a yellow solid. LCMS: C₃₂H₃₅ClFN₅O₄S₂ requires: 671.2, found: m/z=672.2 [M+H]⁺.

### Step-6: Synthesis of tert-butyl 4-[4-(5-{2-[(tert-butoxycarbonyl)amino]pyrimidin-4-yl}-4-[2-fluoro-3-(propane-1-sulfonamido)phenyl]-1,3-thiazol-2-yl)phenyl]piperidine-1-carboxylate (8)

To a mixture of tert-butyl 4-{4-[5-(2-chloropyrimidin-4-yl)-4-[2-fluoro-3-(propane-1-sulfonamido)phenyl]-1,3-thiazol-2-yl]phenyl}piperidine-1-carboxylate (600 mg, 0.89 mmol, 1 equiv) and tert-butyl carbamate (522.80 mg, 4.465 mmol, 5 equiv) in dioxane (6 mL) was added BrettPhos Pd G3 (81 mg, 0.089 mmol, 0.1 equiv) and Cs₂CO₃ (581 mg, 1.78 mmol, 2 equiv) at room temperature under a nitrogen atmosphere. The resulting mixture was stirred for 2 h at 80° C. The resulting mixture was concentrated under reduced pressure. The residue was purified by silica gel column chromatography, eluted with PE:EtOAc (1:1) to afford tert-butyl 4-[4-(5-{2-[(tert-butoxycarbonyl)amino]pyrimidin-4-yl}-4-[2-fluoro-3-(propane-1-sulfonamido)phenyl]-1,3-thiazol-2-yl)phenyl]piperidine-1-carboxylate (400 mg, 53.57%) as a yellow solid. LCMS: C₃₇H₄₅FN₆O₆S₂ requires: 752.2, found: m/z=753.3 [M+H]⁺.

### Step-7: Synthesis of N-{3-[5-(2-aminopyrimidin-4-yl)-2-[4-(piperidin-4-yl)phenyl]-1,3-thiazol-4-yl]-2-fluorophenyl}propane-1-sulfonamide

A mixture of tert-butyl 4-[4-(5-{2-[(tert-butoxycarbonyl)amino]pyrimidin-4-yl}-4-[2-fluoro-3-(propane-1-sulfonamido)phenyl]-1,3-thiazol-2-yl)phenyl]piperidine-1-carboxylate (400 mg, 0.53 mmol, 1 equiv) in HCl/1,4-dioxane (5 mL, 4 M) was stirred overnight at room temperature. The resulting mixture was concentrated under reduced pressure. The crude product was purified by Prep-HPLC with the following conditions Column: XBridge Prep OBD C18 Column, 30*150 mm, 5 m; Mobile Phase A: water (10 mmol/L NH₄HCO₃); Mobile Phase B: ACN; Flow rate: 60 mL/min; Gradient: 30% B to 38% B in 8 min; Wave Length: 220/254 nm; Rₜ₁ (min): 6.75 to afford N-{3-[5-(2-aminopyrimidin-4-yl)-2-[4-(piperidin-4-yl)phenyl]-1,3-thiazol-4-yl]-2-fluorophenyl}propane-1-sulfonamide (223.7 mg, 70.01%) as a yellow solid. LCMS: C₂₇H₂₉FN₆O₂S₂ requires: 552.2, found: m/z=553.2 [M+H]⁺.

¹H NMR (400 MHz, DMSO-d₆) δ 8.15-8.06 (m, 1H), 7.98-7.93 (m, 2H), 7.56-7.52 (m, 1H), 7.40-7.37 (m, 2H), 7.24-7.16 (m, 2H), 6.79 (s, 2H), 6.18-6.16 (m, 1H), 3.21-3.12 (m, 2H), 2.97-2.93 (m, 2H), 2.77-2.69 (m, 3H), 1.80-1.71 (m, 2H), 1.69-1.57 (m, 4H), 0.94-0.90 (m, 3H).

### Synthesis of N-(2-fluoro-3-{5-[2-(methylamino)pyrimidin-4-yl]-2-(piperidin-4-yl)-1,3-thiazol-4-yl}phenyl)propane-1-sulfonamide Hydrochloride Salt

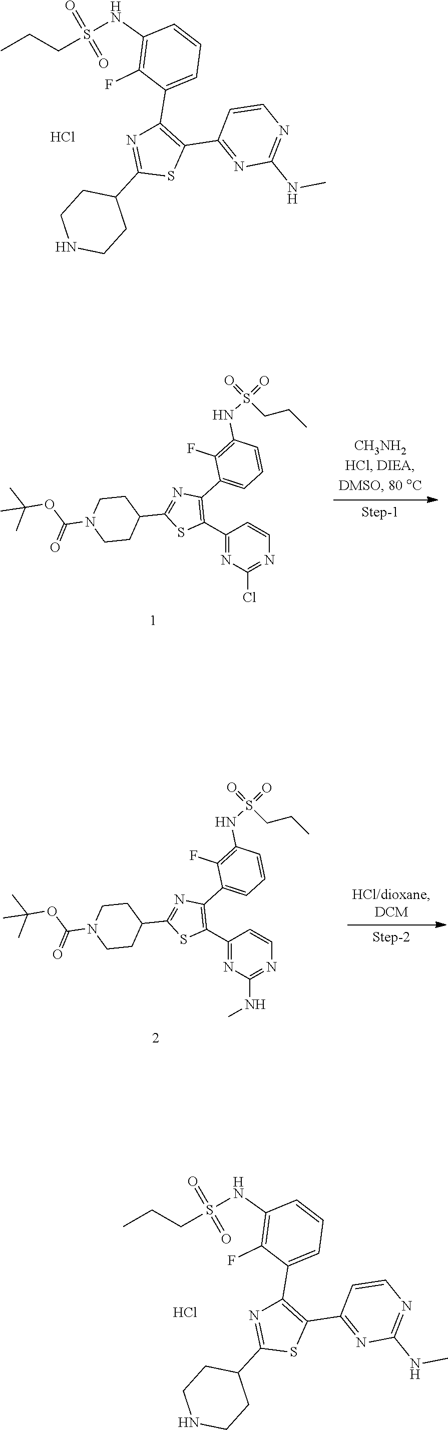

### Step-1: Synthesis of tert-butyl 4-{4-[2-fluoro-3-(propane-1-sulfonamido)phenyl]-5-[2-(methylamino)pyrimidin-4-yl]-1,3-thiazol-2-yl}piperidine-1-carboxylate (2)

To a mixture of tert-butyl 4-[5-(2-chloropyrimidin-4-yl)-4-[2-fluoro-3-(propane-1-sulfonamido)phenyl]-1,3-thiazol-2-yl]piperidine-1-carboxylate (300 mg, 0.503 mmol, 1 equiv) and CH₃NH₂HCl (101.9 mg, 1.509 mmol, 3 equiv) in DMSO (3 mL) was added DIEA (195.1 mg, 1.509 mmol, 3 equiv) at room temperature. The resulting mixture was stirred at 80° C. overnight. The residue was purified by reverse phase column chromatography eluted with CH₃CN:H₂O (1:1) to afford tert-butyl 4-{4-[2-fluoro-3-(propane-1-sulfonamido)phenyl]-5-[2-(methylamino)pyrimidin-4-yl]-1,3-thiazol-2-yl}piperidine-1-carboxylate (280 mg, 94.19%) as a light yellow solid. LCMS: C₂₇H₃₅FN₆O₄S₂ requires: 590.2, found: m/z=591.3. [M+H]⁺.

### Step-2: Synthesis of N-(2-fluoro-3-{5-[2-(methylamino)pyrimidin-4-yl]-2-(piperidin-4-yl)-1,3-thiazol-4-yl}phenyl)propane-1-sulfonamide Hydrochloride

To a mixture of tert-butyl 4-{4-[2-fluoro-3-(propane-1-sulfonamido)phenyl]-5-[2-(methylamino)pyrimidin-4-yl]-1,3-thiazol-2-yl}piperidine-1-carboxylate (280 mg, 0.474 mmol, 1 equiv) in DCM (3 mL) was added HCl (gas) in 1,4-dioxane (3 mL, 4N) at room temperature. The resulting mixture was stirred at room temperature overnight. The resulting mixture was concentrated under reduced pressure. This resulted in N-(2-fluoro-3-{5-[2-(methylamino)pyrimidin-4-yl]-2-(piperidin-4-yl)-1,3-thiazol-4-yl}phenyl)propane-1-sulfonamide hydrochloride salt (174.0 mg, crude) as a yellow solid. LCMS: C₂₂H₂₇FN₆O₂S₂ requires: 490.2, found: m/z=491.2. [M+H]⁺.

¹H NMR (400 MHz, CD₃OD) δ 8.14 (s, 1H), 7.72-7.62 (m, 1H), 7.49-7.43 (m, 1H), 7.42-7.35 (m, 1H), 6.72 (s, 1H), 3.72-3.49 (m, 3H), 3.31-3.20 (m, 2H), 3.20-3.12 (m, 2H), 2.98 (s, 3H), 2.51-2.36 (m, 2H), 2.26-2.05 (m, 2H), 1.98-1.76 (m, 2H), 1.16-1.00 (m, 3H).

### Synthesis of N-{2-fluoro-3-[5-(2-{[(2R)-1-hydroxypropan-2-yl]amino]pyrimidin-4-yl)-2-(piperidin-4-yl)-1,3-thiazol-4-yl}phenyl}propane-1-sulfonamide Hydrochloride Salt

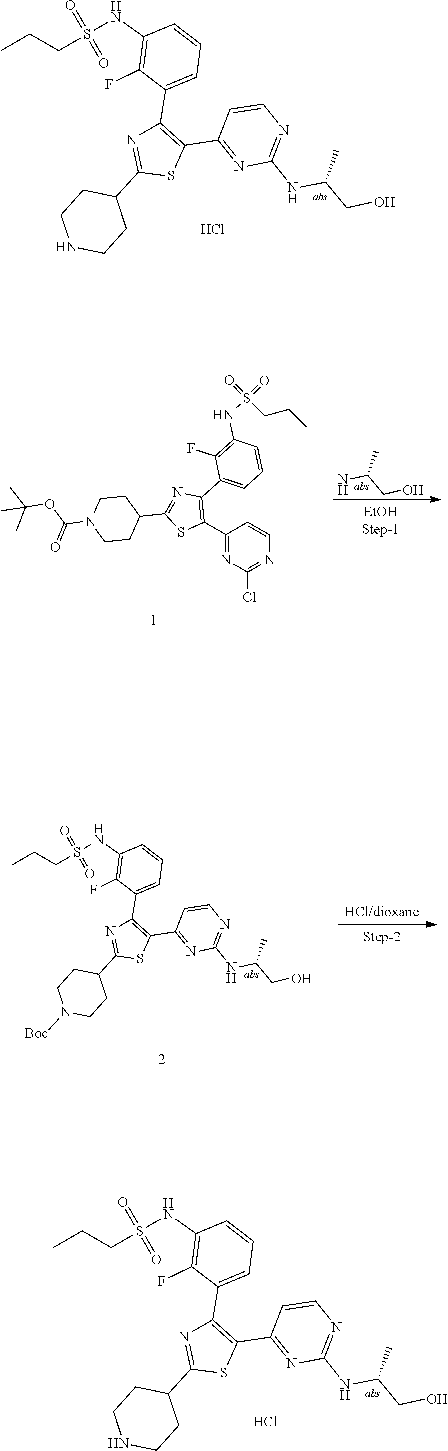

### Step-1: Synthesis of tert-butyl 4-{4-[2-fluoro-3-(propane-1-sulfonamido)phenyl]-5-(2-{[(2R)-1-hydroxypropan-2-yl]amino}pyrimidin-4-yl)-1,3-thiazol-2-yl}piperidine-1-carboxylate (2)

To a mixture of tert-butyl 4-[5-(2-chloropyrimidin-4-yl)-4-[2-fluoro-3-(propane-1-sulfonamido)phenyl]-1,3-thiazol-2-yl]piperidine-1-carboxylate (300 mg, 0.503 mmol, 1 equiv) in EtOH (2 mL) was added (R)-(−)-2-amino-1-propanol (113 mg, 1.509 mmol, 3 equiv) at room temperature. The resulting mixture was stirred overnight at 70° C. The resulting mixture was concentrated under reduced pressure. The residue was purified by reverse phase flash chromatography with H₂O:MeCN (3:1) to afford tert-butyl 4-{4-[2-fluoro-3-(propane-1-sulfonamido)phenyl]-5-(2-{[(2R)-1-hydroxypropan-2-yl]amino}pyrimidin-4-yl)-1,3-thiazol-2-yl}piperidine-1-carboxylate (210 mg, 62.45%) as a yellow solid. LCMS: C₂₉H₃₉FN₆O₅S₂ requires: 634.2, found: m/z=635.3 [M+H]⁺.

### Step-2: Synthesis of N-{2-fluoro-3-[5-(2-{[(2R)-1-hydroxypropan-2-yl]amino}pyrimidin-4-yl)-2-(piperidin-4-yl)-1,3-thiazol-4-yl]phenyl}propane-1-sulfonamide Hydrochloride

A mixture of tert-butyl 4-{4-[2-fluoro-3-(propane-1-sulfonamido)phenyl]-5-(2-{[(2R)-1-hydroxypropan-2-yl]amino}pyrimidin-4-yl)-1,3-thiazol-2-yl}piperidine-1-carboxylate (210 mg, 0.331 mmol, 1 equiv) in HCl/1,4-dioxane (10 mL, 4 M) was stirred overnight at room temperature. The resulting mixture was concentrated under reduced pressure to afford N-{2-fluoro-3-[5-(2-{[(2R)-1-hydroxypropan-2-yl]amino}pyrimidin-4-yl)-2-(piperidin-4-yl)-1,3-thiazol-4-yl]phenyl}propane-1-sulfonamide hydrochloride salt (150.4 mg, crude) as a yellow solid. LCMS: C₂₄H₃₁FN₆O₃S₂ requires: 534.2, found: m/z=535.2 [M+H]⁺.

¹H NMR (400 MHz, CD₃OD) δ 8.14 (s, 1H), 7.80-7.69 (m, 1H), 7.47-7.40 (m, 1H), 7.38-7.30 (m, 1H), 6.71 (s, 1H), 4.05 (s, 1H), 3.83-3.45 (m, 5H), 3.33-3.24 (m, 2H), 3.22-3.15 (m, 2H), 2.46-2.43 (m, 2H), 2.21-2.14 (m, 2H), 1.90-1.84 (m, 2H), 1.28-1.21 (m, 3H), 1.08-1.04 (m, 3H).

### Synthesis of N-{2-fluoro-3-[2-methyl-5-(2-{[4-(piperidin-4-yl)phenyl]amino}pyrimidin-4-yl)-1,3-thiazol-4-yl]phenyl}propane-1-sulfonamide Hydrochloride Salt

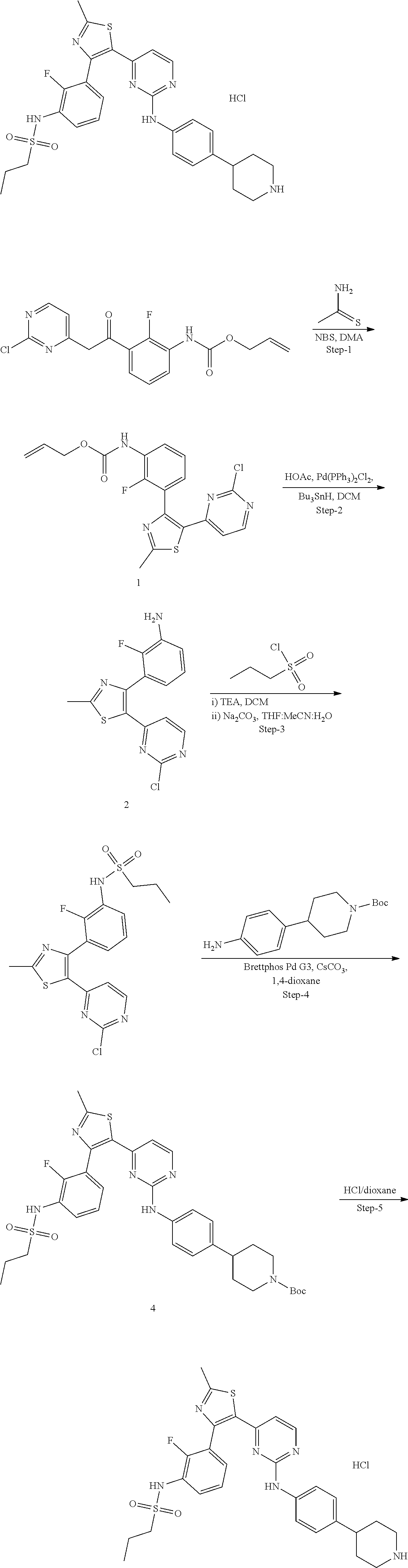

### Step-1: Synthesis of prop-2-en-1-yl N-{3-[5-(2-chloropyrimidin-4-yl)-2-methyl-1,3-thiazol-4-yl]-2-fluorophenyl}carbamate (1)

To a mixture of prop-2-en-1-yl N-{3-[2-(2-chloropyrimidin-4-yl)acetyl]-2-fluorophenyl}carbamate (2 g, 5.718 mmol, 1 equiv) in DMA (10 mL) was added NBS (1.02 g, 5.731 mmol, 1 equiv) and the mixture was stirred for 15 min. Then, thioacetamide (516 mg, 6.862 mmol, 1.2 equiv) was added. The resulting mixture was stirred at 60° C. for 2 h. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with PE:EtOAc (1:2) to afford prop-2-en-1-yl N-{3-[5-(2-chloropyrimidin-4-yl)-2-methyl-1,3-thiazol-4-yl]-2-fluorophenyl}carbamate (1 g, 35.42%) as a yellow solid. LCMS: C₁₈H₁₄ClFN₄O₂S requires: 404.1, found: m/z=405.1 [M+H]⁺.

### Step-2: Synthesis of 3-[5-(2-chloropyrimidin-4-yl)-2-methyl-1,3-thiazol-4-yl]-2-fluoroaniline (2)

To a mixture of prop-2-en-1-yl N-{3-[5-(2-chloropyrimidin-4-yl)-2-methyl-1,3-thiazol-4-yl]-2-fluorophenyl}carbamate (1 g, 2.47 mmol, 1 equiv) in DCM (10 mL) was added HOAc (356 mg, 5.928 mmol, 2.4 equiv) and Pd(PPh₃)₂Cl₂ (34.68 mg, 0.049 mmol, 0.02 equiv). Then, n-Bu₃SnH (1.08 g, 3.705 mmol, 1.5 equiv) was added at 0° C. under a nitrogen atmosphere. The resulting mixture was stirred at room temperature for one hour. The reaction was quenched with saturated aqueous NaHCO₃ at 0° C. The resulting mixture was concentrated under vacuum. The residue was purified by silica gel column chromatography eluted with PE:EtOAc (1:1) to afford 3-[5-(2-chloropyrimidin-4-yl)-2-methyl-1,3-thiazol-4-yl]-2-fluoroaniline (700 mg, 79.51%) as a yellow solid. LCMS: C₁₄H₁₀ClFN₄S requires: 320.0, found: m/z=321.0 [M+H]⁺.

### Step-3: Synthesis of N-{3-[5-(2-chloropyrimidin-4-yl)-2-methyl-1,3-thiazol-4-yl]-2-fluorophenyl}propane-1-sulfonamide

To a mixture of 3-[5-(2-chloropyrimidin-4-yl)-2-methyl-1,3-thiazol-4-yl]-2-fluoroaniline (150 mg, 0.468 mmol, 1 equiv) and TEA (142 mg, 1.404 mmol, 3 equiv) in DCM (10 mL) was added propane-1-sulfonyl chloride (200 mg, 1.404 mmol, 3 equiv) at 0° C. The resulting mixture was stirred at room temperature for one hour. The resulting mixture was concentrated under vacuum. To the above mixture was added THF (5 mL), ACN (5 mL), and Na₂CO₃ (743 mg, 7.02 mmol, 15 equiv) in H₂O (5 mL). The resulting mixture was stirred at 50° C. overnight. The resulting mixture was concentrated under vacuum. The residue was purified by reverse phase flash chromatography eluted with ACN:H₂O (1:1) to afford N-{3-[5-(2-chloropyrimidin-4-yl)-2-methyl-1,3-thiazol-4-yl]-2-fluorophenyl}propane-1-sulfonamide (113.4 mg, 56.63%) as a light yellow solid. LCMS: C₁₇H₁₆ClFN₄O₂S₂ requires: 426.0, found: m/z=427.0 [M+H]⁺.

¹H NMR (400 MHz, CD₃OD) δ 8.48 (d, J=5.2 Hz, 1H), 7.76-7.63 (m, 1H), 7.50-7.30 (m, 2H), 7.15-7.02 (m, 1H), 3.19-3.07 (m, 2H), 2.81 (s, 3H), 1.93-1.74 (m, 2H), 1.13-0.94 (m, 3H).

### Step-4: Synthesis of tert-butyl 4-{4-[(4-{4-[2-fluoro-3-(propane-1-sulfonamido)phenyl]-2-methyl-1,3-thiazol-5-yl}pyrimidin-2-yl)amino]phenyl}piperidine-1-carboxylate (4)

To a mixture of N-{3-[5-(2-chloropyrimidin-4-yl)-2-methyl-1,3-thiazol-4-yl]-2-fluorophenyl}propane-1-sulfonamide (500 mg, 1.171 mmol, 1 equiv) and tert-butyl 4-(4-aminophenyl)piperidine-1-carboxylate (647 mg, 2.342 mmol, 2 equiv) in 1,4-dioxane (10 mL) was added Cs₂CO₃ (763 mg, 2.342 mmol, 2 equiv) and BrettPhos Pd G3 (106 mg, 0.117 mmol, 0.1 equiv). The resulting mixture was stirred at 80° C. for 2 h under a nitrogen atmosphere. The resulting mixture was concentrated under vacuum. The residue was purified by silica gel column chromatography eluted with PE:EtOAc (1:1) to afford tert-butyl 4-{4-[(4-{4-[2-fluoro-3-(propane-1-sulfonamido)phenyl]-2-methyl-1,3-thiazol-5-yl}pyrimidin-2-yl)amino]phenyl}piperidine-1-carboxylate (500 mg, 57.62%) as a yellow solid. LCMS: C₃₃H₃₉FN₆O₄S₂ requires: 666.3, found: m/z=667.3 [M+H]⁺.

### Step-5: Synthesis of N-{2-fluoro-3-[2-methyl-5-(2-{[4-(piperidin-4-yl)phenyl]amino}pyrimidin-4-yl)-1,3-thiazol-4-yl]phenyl}propane-1-sulfonamide Hydrochloride

To a mixture of tert-butyl 4-{4-[(4-{4-[2-fluoro-3-(propane-1-sulfonamido)phenyl]-2-methyl-1,3-thiazol-5-yl}pyrimidin-2-yl)amino]phenyl}piperidine-1-carboxylate (1.0 g, 1.5 mmol, 1 equiv) in DCM (10 mL) was added HCl (gas) in 1,4-dioxane (5 mL, 4N) at 0° C. The resulting mixture was stirred overnight at room temperature. The resulting mixture was concentrated under vacuum. The residue was purified by reverse phase flash chromatography eluted with ACN:H₂O (1:1) to afford N-{2-fluoro-3-[2-methyl-5-(2-{[4-(piperidin-4-yl)phenyl]amino}pyrimidin-4-yl)-1,3-thiazol-4-yl]phenyl}propane-1-sulfonamide hydrochloride salt (561.3 mg, 61.12%) as a yellow solid. LCMS: C₂₈H₃₁FN₆O₂S₂ requires: 566.2, found: m/z=567.2 [M+H]⁺. ¹H NMR (400 MHz, CD₃COOD) δ 8.24 (d, J=5.6 Hz, 1H), 7.82-7.70 (m, 1H), 7.68-7.59 (m, 2H), 7.56-7.46 (m, 1H), 7.44-7.34 (m, 1H), 7.32-7.20 (m, 2H), 6.62 (d, J=5.6 Hz, 1H), 3.77-3.63 (m, 2H), 3.34-3.16 (m, 2H), 3.14-3.00 (m, 2H), 2.87 (s, 4H), 2.11 (s, 4H), 1.87-1.72 (m, 2H), 1.07-0.94 (m, 3H).

### Synthesis of N-{3-[5-(2-aminopyrimidin-4-yl)-2-[2-(piperidin-4-yl)ethyl]-1,3-thiazol-4-yl]-2-fluorophenyl}propane-1-sulfonamide Hydrochloride Salt

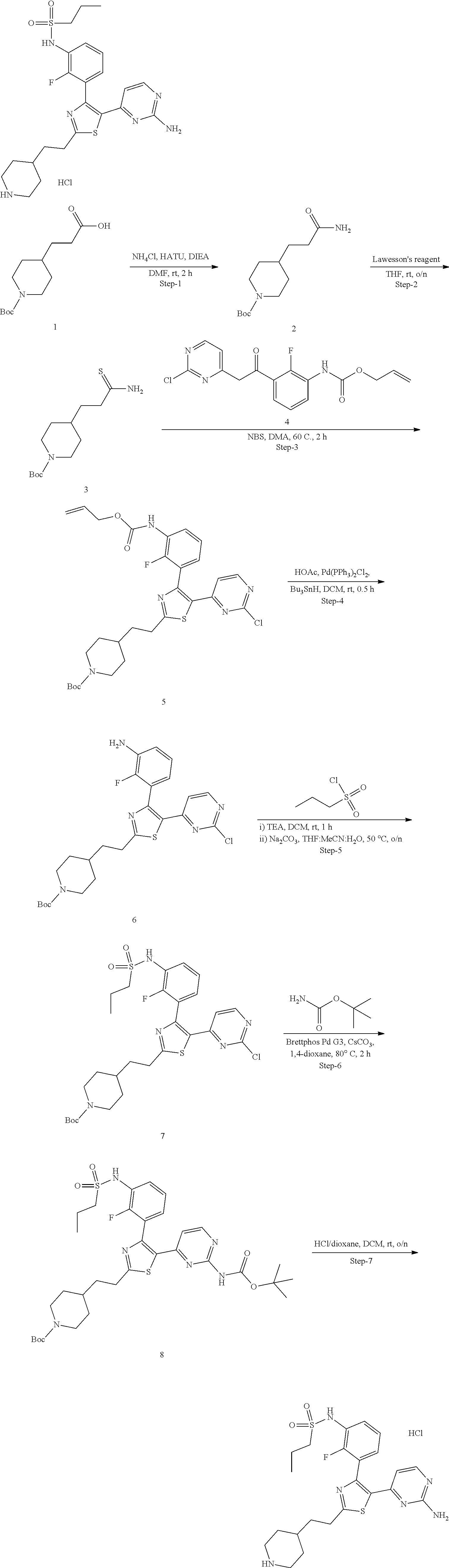

### Step-1: Synthesis of tert-butyl 4-(2-carbamoylethyl)piperidine-1-carboxylate (2)

To a mixture of 3-[1-(tert-butoxycarbonyl)piperidin-4-yl]propanoic acid (5.0 g, 19.430 mmol, 1 equiv), HATU (11.08 g, 29.145 mmol, 1.5 equiv), and NH₄Cl (5.19 g, 97.150 mmol, 5.0 equiv) in DMF (50 mL) was added DIEA (7.53 g, 58.290 mmol, 3.0 equiv) dropwise at room temperature. The resulting mixture was stirred for 2 h at room temperature. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with PE:EtOAc (2:1) to afford tert-butyl 4-(2-carbamoylethyl)piperidine-1-carboxylate (3.2 g, 64.25%) as a white solid. LCMS: C₁₃H₂₄N₂O₃ requires: 256.2, found: m/z=257.2 [M+H]⁺.

### Step-2: Synthesis of tert-butyl 4-(2-carbomethoxyethyl)piperidine-1-carboxylate (3)

To a mixture of tert-butyl 4-(2-carbamoylethyl)piperidine-1-carboxylate (3.0 g, 11.703 mmol, 1 equiv) in THF (30 mL) was added Lawesson's Reagent (2.36 g, 5.851 mmol, 0.5 equiv). The resulting mixture was stirred for at 50° C. overnight. The reaction was quenched by the addition of saturated aqueous sodium hyposulfite at 0° C. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with CH₂Cl₂:MeOH (1:1) to afford tert-butyl 4-(2-carbomethoxyethyl)piperidine-1-carboxylate (1.5 g, 47.05%) as a white solid. LCMS: C₁₃H₂₄N₂O₂S requires: 272.2, found: m/z=273.2 [M+H]⁺.

### Step-3: Synthesis of tert-butyl 4-{2-[5-(2-chloropyrimidin-4-yl)-4-(2-fluoro-3-{[(prop-2-en-1-yloxy)carbonyl]amino}phenyl)-1,3-thiazol-2-yl]ethyl}piperidine-1-carboxylate (5)

To a mixture of prop-2-en-1-yl N-{3-[2-(2-chloropyrimidin-4-yl)acetyl]-2-fluorophenyl}carbamate (1.92 g, 5.506 mmol, 1.00 equiv) in DMA (15 mL) was added NBS (980.06 mg, 5.506 mmol, 1.0 equiv) for 15 min at room temperature followed by the addition of tert-butyl 4-(2-carbamothioylethyl)piperidine-1-carboxylate (1.5 g, 5.506 mmol, 1.00 equiv) at room temperature. The resulting mixture was stirred for 2 h at 60° C. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with CH₂Cl₂:MeOH (6:1) to afford tert-butyl 4-{2-[5-(2-chloropyrimidin-4-yl)-4-(2-fluoro-3-{[(prop-2-en-1-yloxy)carbonyl]amino}phenyl)-1,3-thiazol-2-yl]ethyl}piperidine-1-carboxylate (1.2 g, 36.19%) as a white solid. LCMS: C₂₉H₃₃ClFN₅O₄S requires: 601.2, found: m/z=602.2 [M+H]⁺.

### Step-4: Synthesis of tert-butyl 4-{2-[4-(3-amino-2-fluorophenyl)-5-(2-chloropyrimidin-4-yl)-1,3-thiazol-2-yl]ethyl}piperidine-1-carboxylate (6)

To a mixture of tert-butyl 4-{2-[5-(2-chloropyrimidin-4-yl)-4-(2-fluoro-3-{[(prop-2-en-1-yloxy)carbonyl]amino}phenyl)-1,3-thiazol-2-yl]ethyl}piperidine-1-carboxylate (1.2 g, 1.993 mmol, 1 equiv) and HOAc (0.30 g, 4.982 mmol, 2.5 equiv) in DCM (12 mL) was added n-Bu₃SnH (0.93 g, 3.189 mmol, 1.6 equiv) and Pd(PPh₃)₂Cl₂ (27.98 mg, 0.040 mmol, 0.02 equiv) at room temperature. The resulting mixture was stirred for 0.5 h at room temperature under a nitrogen atmosphere. The resulting mixture was extracted with DCM. The combined organic layers were washed with water and dried over anhydrous Na₂SO₄. After filtration, the filtrate was concentrated under reduced pressure to afford tert-butyl 4-{2-[4-(3-amino-2-fluorophenyl)-5-(2-chloropyrimidin-4-yl)-1,3-thiazol-2-yl]ethyl}piperidine-1-carboxylate (800 mg, crude) as a yellow oil. The crude product was used in the next step directly without further purification. LCMS: C₂₅H₂₉ClFN₅O₂S requires: 517.2, found: m/z=518.2 [M+H]⁺.

### Step-5: Synthesis of tert-butyl 4-{2-[5-(2-chloropyrimidin-4-yl)-4-[2-fluoro-3-(propane-1-sulfonamido)phenyl]-1,3-thiazol-2-yl]ethyl}piperidine-1-carboxylate (7)

To a mixture of tert-butyl 4-{2-[4-(3-amino-2-fluorophenyl)-5-(2-chloropyrimidin-4-yl)-1,3-thiazol-2-yl]ethyl}piperidine-1-carboxylate (800 mg, 1.544 mmol, 1 equiv) and TEA (468.80 mg, 4.632 mmol, 3.0 equiv) in DCM (10 mL) was added propane-1-sulfonyl chloride (440.42 mg, 3.088 mmol, 2.0 equiv) at room temperature. The resulting mixture was stirred for one hour at room temperature. The resulting mixture was concentrated under reduced pressure. The crude product was dissolved in THF (3 mL), ACN (3 mL), and H₂O (3 mL). Na₂CO₃ (799.92 mg, 7.545 mmol, 6.89 equiv) was added at room temperature. The resulting mixture was stirred at 50° C. overnight. The resulting mixture was concentrated under reduced pressure. The residue was purified by silica gel column chromatography eluted with CH₂Cl₂:MeOH (10:1) to afford tert-butyl 4-{2-[5-(2-chloropyrimidin-4-yl)-4-[2-fluoro-3-(propane-1-sulfonamido)phenyl]-1,3-thiazol-2-yl]ethyl}piperidine-1-carboxylate (680 mg, 85.00%) as a yellow solid. LCMS: C₂₈H₃₅ClFN₅O₄S₂ requires: 623.2, found: m/z=624.2 [M+H]⁺.

### Step-6: Synthesis of tert-butyl 4-{2-[5-(2-chloropyrimidin-4-yl)-4-(2-fluoro-3-{[(prop-2-en-1-yloxy)carbonyl]amino}phenyl)-1,3-thiazol-2-yl]ethyl}piperidine-1-carboxylate (8)

To a mixture of tert-butyl 4-{2-[5-(2-chloropyrimidin-4-yl)-4-[2-fluoro-3-(propane-1-sulfonamido)phenyl]-1,3-thiazol-2-yl]ethyl}piperidine-1-carboxylate (660 mg, 1.057 mmol, 1 equiv) and tert-butyl carbamate (619.34 mg, 5.285 mmol, 5.0 equiv) in 1,4-dioxane (7 mL) was added Cs₂CO₃ (689.02 mg, 2.114 mmol, 2.0 equiv) and BrettPhos Pd G₃ (95.85 mg, 0.106 mmol, 0.1 equiv) at room temperature under a nitrogen atmosphere. The resulting mixture was stirred for 2 h at 80° C. under the nitrogen atmosphere. The resulting mixture was concentrated under reduced pressure. The residue was purified by silica gel column chromatography eluted with CH₂Cl₂:MeOH (10:1) to afford tert-butyl 4-[2-(5-{2-[(tert-butoxycarbonyl)amino]pyrimidin-4-yl}-4-[2-fluoro-3-(propane-1-sulfonamido)phenyl]-1,3-thiazol-2-yl)ethyl]piperidine-1-carboxylate (400 mg, 53.67%) as a white solid. LCMS: C₃₃H₄₅FN₆O₆S₂ requires: 704.3, found: m/z=705.3 [M+H]⁺.

### Step-7: Synthesis of N-{3-[5-(2-aminopyrimidin-4-yl)-2-[2-(piperidin-4-yl)ethyl]-1,3-thiazol-4-yl]-2-fluorophenyl}propane-1-sulfonamide Hydrochloride

To a mixture of tert-butyl 4-[2-(5-{2-[(tert-butoxycarbonyl)amino]pyrimidin-4-yl}-4-[2-fluoro-3-(propane-1-sulfonamido)phenyl]-1,3-thiazol-2-yl)ethyl]piperidine-1-carboxylate (380 mg, 0.539 mmol, 1 equiv) in DCM (2 mL) was added HCl (gas) in 1,4-dioxane (6 mL, 4 M). The resulting mixture was stirred at room temperature overnight. The resulting mixture was concentrated under reduced pressure. The residue was purified by reversed-phase flash chromatography with the following conditions: column, C18 silica gel; mobile phase, MeCN in water (0.1% NH₄HCO₃), 10% to 50% gradient in 10 min; detector, UV 254 nm to afford N-{3-[5-(2-aminopyrimidin-4-yl)-2-[2-(piperidin-4-yl)ethyl]-1,3-thiazol-4-yl]-2-fluorophenyl}propane-1-sulfonamide hydrochloride salt (205.9 mg, 67.55%) as a yellow solid. LCMS: C₂₃H₂₉FN₆O₂S₂ requires: 504.2, found: m/z=505.2 [M+H]⁺. ¹H NMR (400 MHz, CD₃OD) δ 8.00 (s, 1H), 7.62-7.58 (m, 1H), 7.39-7.19 (m, 1H), 6.58-6.47 (m, 1H), 3.82-3.42 (m, 1H), 3.36-3.25 (m, 2H), 3.14-2.98 (m, 4H), 2.94-2.86 (m, 2H), 2.03-1.90 (m, 2H), 1.82-1.65 (m, 5H), 1.48-1.31 (m, 2H), 0.97-0.93 (m, 3H).

### Synthesis of N-(3-{5-[2-(cyclopropylamino)pyrimidin-4-yl]-2-(piperidin-4-yl)-1,3-thiazol-4-yl}-2-fluorophenyl)propane-1-sulfonamide Hydrochloride Salt

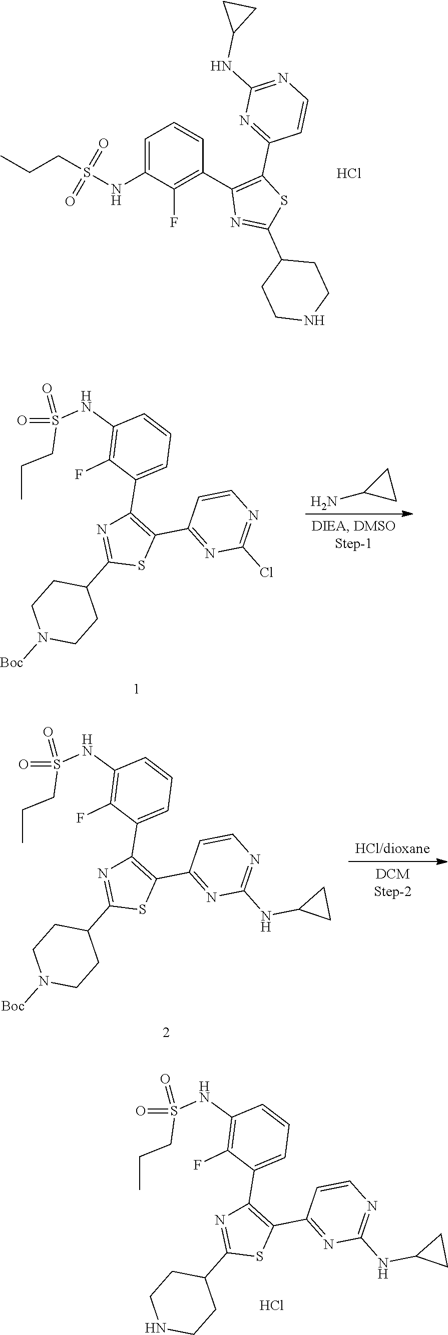

### Step-1: Synthesis of tert-butyl 4-{5-[2-(cyclopropylamino)pyrimidin-4-yl]-4-[2-fluoro-3-(propane-1-sulfonamido)phenyl]-1,3-thiazol-2-yl}piperidine-1-carboxylate

To a mixture of tert-butyl 4-[5-(2-chloropyrimidin-4-yl)-4-[2-fluoro-3-(propane-1-sulfonamido)phenyl]-1,3-thiazol-2-yl]piperidine-1-carboxylate (500 mg, 0.83 mmol, 1 equiv) and DIEA (325 mg, 2.51 mmol, 3 equiv) in DMSO (5 mL) was added aminocyclopropane (143 mg, 2.51 mmol, 3 equiv) at room temperature. The resulting mixture was stirred overnight at 80° C. The resulting mixture was extracted with EtOAc. The combined organic layers were washed with water and dried over anhydrous Na₂SO₄. After filtration, the filtrate was concentrated under reduced pressure. The residue was purified by reverse phase flash chromatography with H₂O:MeCN (1:1) to afford tert-butyl 4-{5-[2-(cyclopropylamino)pyrimidin-4-yl]-4-[2-fluoro-3-(propane-1-sulfonamido)phenyl]-1,3-thiazol-2-yl}piperidine-1-carboxylate (400 mg, 75.78%) as a yellow solid. LCMS: C₂₉H₃₇FN₆O₄S₂ requires: 616.2, found: m/z=617.3 [M+H]⁺.

### Step-2: Synthesis of N-(3-{5-[2-(cyclopropylamino)pyrimidin-4-yl]-2-(piperidin-4-yl)-1,3-thiazol-4-yl}-2-fluorophenyl)propane-1-sulfonamide Hydrochloride

To a mixture of tert-butyl 4-{5-[2-(cyclopropylamino)pyrimidin-4-yl]-4-[2-fluoro-3-(propane-1-sulfonamido)phenyl]-1,3-thiazol-2-yl}piperidine-1-carboxylate (400 mg, 0.649 mmol, 1 equiv) in DCM (10 mL) was added HCl in 1,4-dioxane (1 mL, 4 M) at room temperature. The resulting mixture was stirred overnight at room temperature. The resulting mixture was concentrated under reduced pressure to afford N-(3-{5-[2-(cyclopropylamino)pyrimidin-4-yl]-2-(piperidin-4-yl)-1,3-thiazol-4-yl}-2-fluorophenyl)propane-1-sulfonamide hydrochloride salt (213.6 mg, crude) as a yellow solid. LCMS: C₂₄H₂₉FN₆O₂S₂ requires: 516.2, found: m/z=517.2 [M+H]⁺. ¹H NMR (400 MHz, CD₃OD) δ 8.13 (d, J=6.8 Hz, 1H), 7.67-7.65 (m, 1H), 7.45-7.43 (m, 1H), 7.38-7.36 (m, 1H), 6.66 (d, J=6.8 Hz, 1H), 3.56-3.52 (m, 3H), 3.24-3.12 (m, 4H), 2.71-2.69 (m, 1H), 2.42-2.38 (m, 2H), 2.14-2.09 (m, 2H), 1.88-1.82 (m, 2H), 1.06-1.00 (m, 5H), 0.83-0.75 (m, 2H).

### Synthesis of N-(2-fluoro-3-{5-[2-(isopropylamino)pyrimidin-4-yl]-2-(piperidin-4-yl)-1,3-thiazol-4-yl}phenyl)propane-1-sulfonamide Hydrochloride Salt

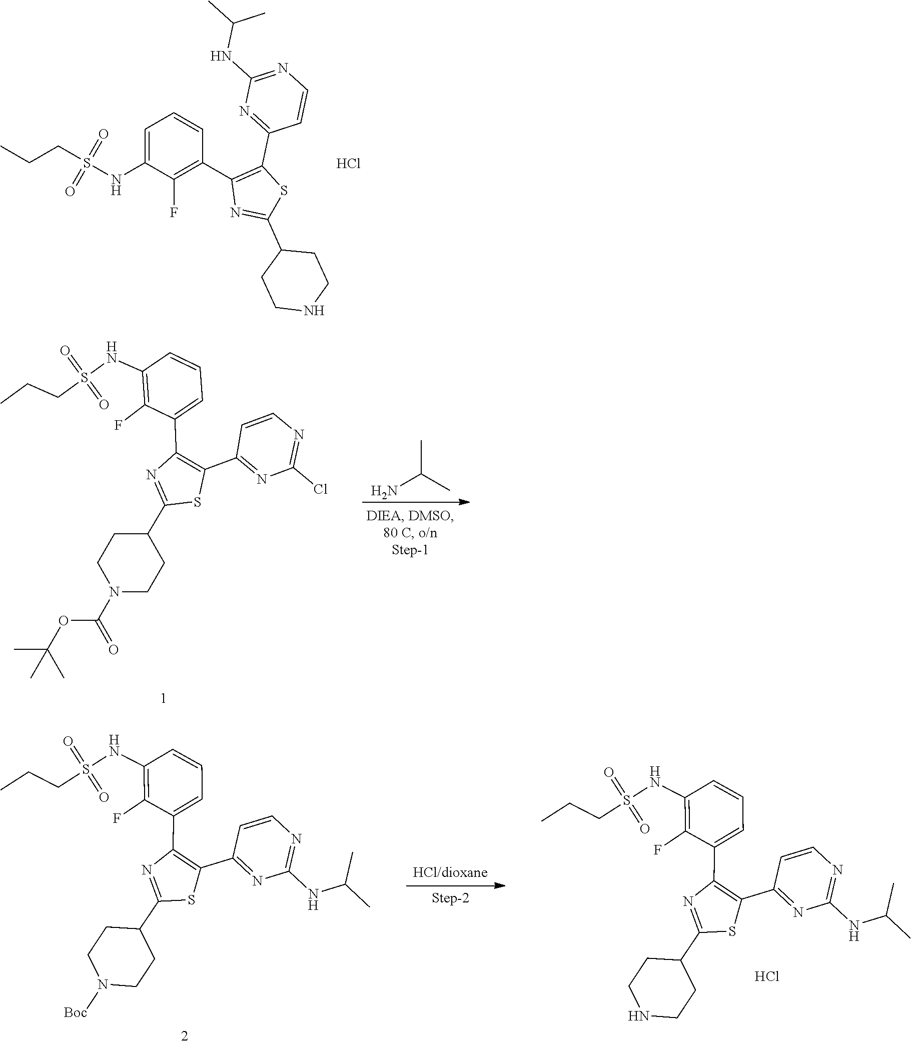

### Step-1: Synthesis of tert-butyl 4-{4-[2-fluoro-3-(propane-1-sulfonamido)phenyl]-5-[2-(isopropylamino)pyrimidin-4-yl]-1,3-thiazol-2-yl}piperidine-1-carboxylate (2)

To a mixture of tert-butyl 4-[5-(2-chloropyrimidin-4-yl)-4-[2-fluoro-3-(propane-1-sulfonamido)phenyl]-1,3-thiazol-2-yl]piperidine-1-carboxylate (500 mg, 0.839 mmol, 1 equiv) and isopropylamine (148.74 mg, 2.517 mmol, 3.0 equiv) in DMSO (5 mL) was added DIEA (325.22 mg, 2.517 mmol, 3.0 equiv) at room temperature. The resulting mixture was stirred at 80° C. overnight. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with CH₂Cl₂:MeOH (10:1) to afford tert-butyl 4-{4-[2-fluoro-3-(propane-1-sulfonamido)phenyl]-5-[2-(isopropylamino)pyrimidin-4-yl]-1,3-thiazol-2-yl}piperidine-1-carboxylate (490 mg, 94.41%) as a yellow solid. LCMS: C₂₉H₃₉FN₆O₄S₂ requires: 618.3, found: m/z=619.1 [M+H]⁺.

### Step-2: Synthesis of N-(2-fluoro-3-{5-[2-(isopropylamino)pyrimidin-4-yl]-2-(piperidin-4-yl)-1,3-thiazol-4-yl}phenyl)propane-1-sulfonamide Hydrochloride

A mixture of tert-butyl 4-{4-[2-fluoro-3-(propane-1-sulfonamido)phenyl]-5-[2-(isopropylamino)pyrimidin-4-yl]-1,3-thiazol-2-yl}piperidine-1-carboxylate (490 mg, 0.792 mmol, 1 equiv) in HCl (gas)/1,4-dioxane (5 mL, 4 M) was stirred at room temperature overnight. The resulting mixture was concentrated under reduced pressure. The residue was purified by C18 reverse phase flash chromatography with ACN:H₂O (0.5% HCl) (1:1) to afford N-(2-fluoro-3-{5-[2-(isopropylamino)pyrimidin-4-yl]-2-(piperidin-4-yl)-1,3-thiazol-4-yl}phenyl)propane-1-sulfonamide hydrochloride salt (185.9 mg, 45.26%) as a yellow solid. LCMS: C₂₄H₃₁FN₆O₂S₂ requires: 518.2, found: m/z=519.2 [M+H]⁺.

¹H NMR (300 MHz, CD₃OD) δ 8.12 (d, J=6.6 Hz, 1H), 7.70-768 (m, 1H), 7.52-7.35 (m, 2H), 6.69 (s, 1H), 4.01 (s, 1H), 3.64-3.52 (m, 3H), 3.30-3.25 (m, 2H), 3.20-3.08 (m, 2H), 2.47-2.43 (m, 2H), 2.28-2.08 (m, 2H), 1.96-1.79 (m, 2H), 1.37-1.23 (m, 6H), 1.10-1.02 (m, 3H).

### Synthesis of N-{2-fluoro-3-[3-(pyridin-4-yl)-1H-pyrazol-4-yl]phenyl}propane-1-sulfonamide

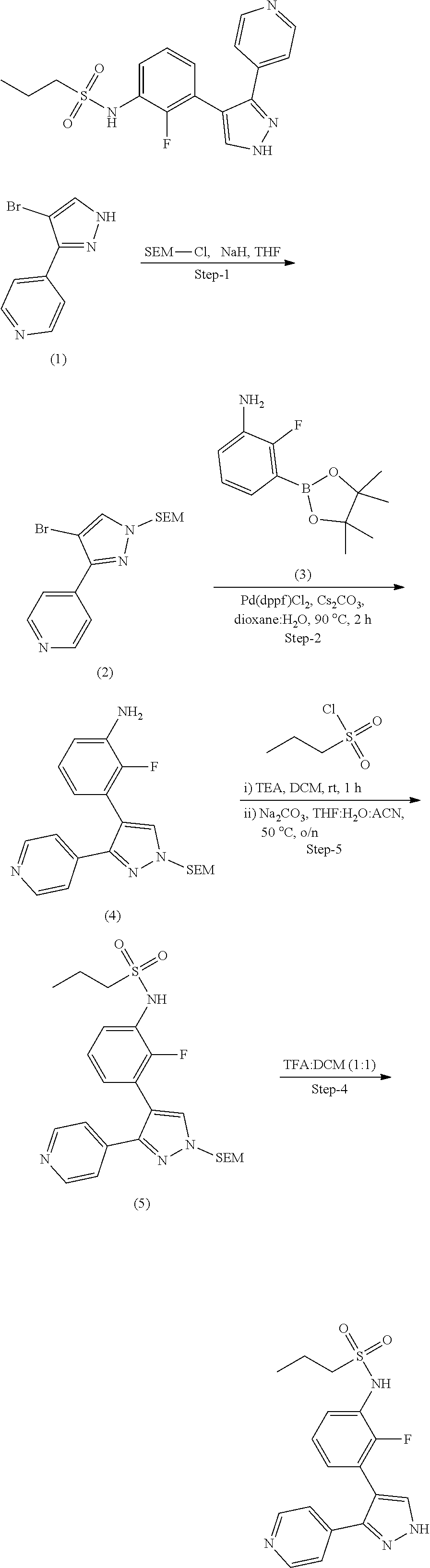

### Step-1: Synthesis of 4-(4-bromo-1-{[2-(trimethylsilyl)ethoxy]methyl}pyrazol-3-yl)pyridin (2)

To a mixture of 4-(4-bromo-1H-pyrazol-3-yl)pyridine (5 g, 22.315 mmol, 1 equiv) in THF (70 mL) was added NaH (1.07 g, 44.630 mmol, 2 equiv, 60% in mineral oil) at 0° C. After the mixture was stirred for 30 min, SEM-Cl (5.58 g, 33.473 mmol, 1.5 equiv) was added at 0° C. The resulting mixture was stirred at room temperature for 2 h. The reaction was quenched with water. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with CH₂Cl₂:MeOH (10:1) to afford 4-(4-bromo-1-{[2-(trimethylsilyl)ethoxy]methyl}pyrazol-3-yl)pyridine (6.8 g, 86.00%) as a yellow oil. MS (ESI) calc'd for (C₁₄H₂₀BrN₃OSi) [M+H]⁺, 354.1; found, 354.0.

### Step-2: Synthesis of 2-fluoro-3-[3-(pyridin-4-yl)-1-{[2-(trimethylsilyl)ethoxy]methyl}pyrazol-4-yl]aniline (4)

To a mixture of 4-(4-bromo-1-{[2-(trimethylsilyl)ethoxy]methyl}pyrazol-3-yl)pyridine (5.0 g, 14.111 mmol, 1 equiv) and 2-fluoro-3-(4,4,5,5-tetramethyl-1,3,2-dioxaborolan-2-yl) aniline (4.01 g, 16.933 mmol, 1.2 equiv) in dioxane (50 mL) and H₂O (10 mL) was added Cs₂CO₃ (9.2 g, 28.222 mmol, 2 equiv) and Pd(dppf)Cl₂·CH₂Cl₂ (2.3 g, 2.822 mmol, 0.2 equiv). The resulting mixture was stirred at 90° C. for 2 h under a nitrogen atmosphere. The resulting mixture was concentrated under reduced pressure. The residue was purified by silica gel column chromatography eluted with CH₂Cl₂:MeOH (10:1) to afford 2-fluoro-3-[3-(pyridin-4-yl)-1-{[2-(trimethylsilyl)ethoxy]methyl}pyrazol-4-yl]aniline (4.2 g, 77.40%) as a yellow solid. MS (ESI) calc'd for (C₂₀H₂₅FN₄OSi) [M+H]⁺, 385.2; found, 385.2.

### Step-3: Synthesis of N-{2-fluoro-3-[3-(pyridin-4-yl)-1-{[2-(trimethylsilyl)ethoxy]methyl}pyrazol-4-yl]phenyl}propane-1-sulfonamide (5)

To a mixture of 2-fluoro-3-[3-(pyridin-4-yl)-1-{[2-(trimethylsilyl)ethoxy]methyl}pyrazol-4-yl]aniline (4.2 g, 10.922 mmol, 1 equiv) and TEA (3.3 g, 32.766 mmol, 3 equiv) in DCM (50 mL) was added propane-1-sulfonyl chloride (3.1 g, 21.844 mmol, 2 equiv) at room temperature. The resulting mixture was stirred at room temperature for one hour. The resulting mixture was concentrated under reduced pressure. The crude product was dissolved in THF (12 mL), ACN (12 mL), and H₂O (12 mL). Then Na₂CO₃ (3.7 g, 35.190 mmol, 6 equiv) was added at room temperature. The resulting mixture was stirred at 50° C. overnight. The bulk of the organic solvents were removed under reduced pressure. The resulting mixture was extracted with DCM. The combined organic layers were washed with water and dried over anhydrous Na₂SO₄. After filtration, the filtrate was concentrated under reduced pressure. The residue was purified by silica gel column chromatography eluted with CH₂Cl₂:MeOH (10:1) to afford N-{2-fluoro-3-[3-(pyridin-4-yl)-1-{[2-(trimethylsilyl)ethoxy]methyl}pyrazol-4-yl]phenyl}propane-1-sulfonamide (2.5 g, 86.88%) as a yellow solid. MS (ESI) calc'd for (C₂₃H₃₁FN₄O₃SSi) [M+H]⁺, 491.2; found, 491.2.

### Step-4: Synthesis of N-{2-fluoro-3-[3-(pyridin-4-yl)-1H-pyrazol-4-yl]phenyl}propane-1-sulfonamide

To a mixture of N-{2-fluoro-3-[3-(pyridin-4-yl)-1-{[2-(trimethylsilyl)ethoxy]methyl}pyrazol-4-yl]phenyl}propane-1-sulfonamide (2.5 g, 5.095 mmol, 1 equiv) in DCM (15 mL) was added TFA (15 mL). The resulting mixture was stirred at room temperature for 2 h. The resulting mixture was concentrated under reduced pressure. The residue was purified by silica gel column chromatography eluted with CH₂Cl₂:MeOH (10:1) to afford N-{2-fluoro-3-[3-(pyridin-4-yl)-1H-pyrazol-4-yl]phenyl}propane-1-sulfonamide (1.55 g, 84.41%) as a yellow solid. MS (ESI) calc'd for (C₁₇H₁₇FN₄O₂S) [M+H]⁺, 361.1; found, 361.0.

### Synthesis of N-(2-fluoro-3-{2-[4-(piperazin-1-yl)phenyl]-5-(pyridin-4-yl)-1,3-thiazol-4-yl}phenyl)propane-1-sulfonamide Hydrochloride Salt

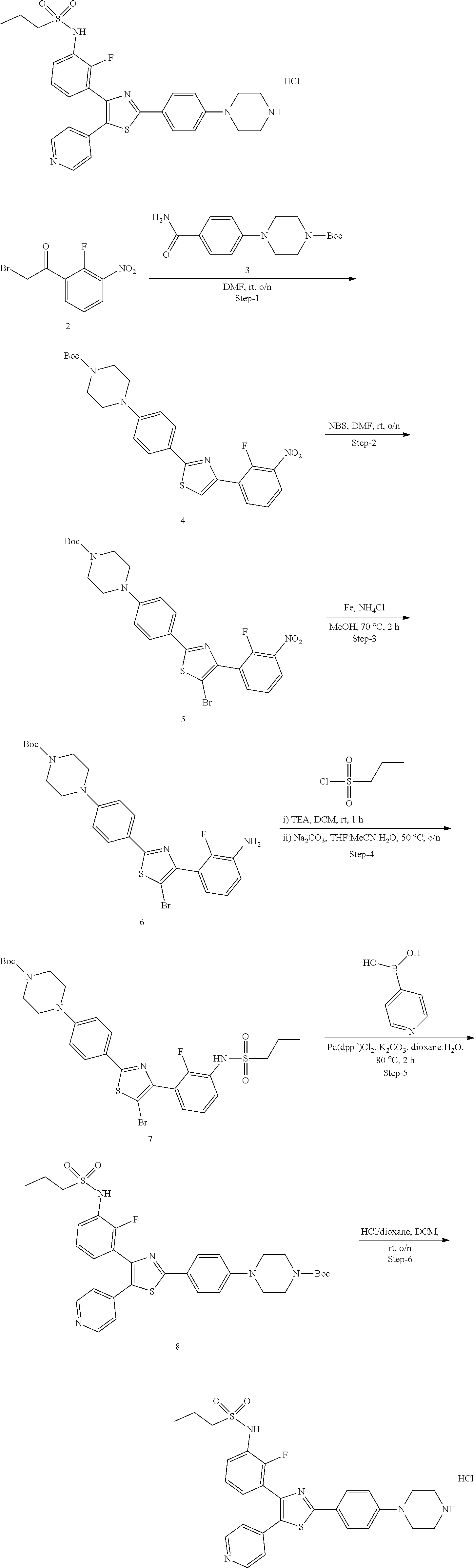

### Step-1: Synthesis of tert-butyl 4-{4-[4-(2-fluoro-3-nitrophenyl)-1,3-thiazol-2-yl]phenyl}piperazine-1-carboxylate (4)

To a mixture of 2-bromo-1-(2-fluoro-3-nitrophenyl)ethanone (2 g, 7.633 mmol, 1 equiv) in DMF (20 mL) was added tert-butyl 4-(4-carbamothioylphenyl)piperazine-1-carboxylate (2.70 g, 8.396 mmol, 1.1 equiv) at room temperature. The resulting mixture was stirred at room temperature overnight. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with PE:EtOAc (2:1) to afford tert-butyl 4-{4-[4-(2-fluoro-3-nitrophenyl)-1,3-thiazol-2-yl]phenyl}piperazine-1-carboxylate (3.2 g, 86.52%) as a yellow solid. LCMS: C₂₄H₂₅FN₄O₄S requires: 484.2, found: m/z=485.2 [M+H]⁺.

### Step-2: Synthesis of tert-butyl 4-{4-[5-bromo-4-(2-fluoro-3-nitrophenyl)-1,3-thiazol-2-yl]phenyl}piperazine-1-carboxylate (5)

To a mixture of tert-butyl 4-{4-[4-(2-fluoro-3-nitrophenyl)-1,3-thiazol-2-yl]phenyl}piperazine-1-carboxylate (3 g, 6.191 mmol, 1 equiv) in DMF (30 mL) was added NBS (1.21 g, 6.810 mmol, 1.1 equiv) at room temperature. The resulting mixture was stirred at room temperature overnight. The resulting mixture was extracted with EtOAc. The combined organic layers were washed with water and dried over anhydrous Na₂SO₄. After filtration, the filtrate was concentrated under reduced pressure. The residue was purified by C18 reverse phase flash chromatography with ACN:H₂O (1:1) to afford tert-butyl 4-{4-[5-bromo-4-(2-fluoro-3-nitrophenyl)-1,3-thiazol-2-yl]phenyl}piperazine-1-carboxylate (900 mg, 25.8%) as a yellow solid. LCMS: C₂₄H₂₄BrFN₄O₄S requires: 562.1, found: m/z=563.1 [M+H]⁺.

### Step-3: Synthesis of tert-butyl 4-{4-[4-(3-amino-2-fluorophenyl)-5-bromo-1,3-thiazol-2-yl]phenyl}piperazine-1-carboxylate (6)

To a mixture of tert-butyl 4-{4-[5-bromo-4-(2-fluoro-3-nitrophenyl)-1,3-thiazol-2-yl]phenyl}piperazine-1-carboxylate (900 mg, 1.588 mmol, 1 equiv) in saturated aqueous NH₄Cl (2 mL) and MeOH (8 mL) was added Fe (55.75 mg, 0.995 mmol, 0.6 equiv) in portions at room temperature. The resulting mixture was stirred for 2 h at 70° C. The resulting mixture was concentrated under reduced pressure. The residue was purified by C18 reverse phase flash chromatography with ACN:H₂O (1:1) to afford tert-butyl 4-{4-[4-(3-amino-2-fluorophenyl)-5-bromo-1,3-thiazol-2-yl]phenyl}piperazine-1-carboxylate (800 mg, 94.44%) as a yellow solid. LCMS: C₂₄H₂₆BrFN₄O₂S requires: 532.1, found: m/z=533.1 [M+H]⁺.

### Step-4: Synthesis of tert-butyl 4-(4-{5-bromo-4-[2-fluoro-3-(propane-1-sulfonamido)phenyl]-1,3-thiazol-2-yl}phenyl)piperazine-1-carboxylate (7)

To a mixture of tert-butyl 4-{4-[4-(3-amino-2-fluorophenyl)-5-bromo-1,3-thiazol-2-yl]phenyl}piperazine-1-carboxylate (800 mg, 1.500 mmol, 1 equiv) and TEA (455.26 mg, 4.500 mmol, 3.0 equiv) in DCM (10 mL) was added propane-1-sulfonyl chloride (320.77 mg, 2.250 mmol, 1.5 equiv) in portions at 0° C. The resulting mixture was stirred for one hour at room temperature. The resulting mixture was concentrated under reduced pressure. The crude product was dissolved in THF (3 mL), ACN (3 mL), and H₂O (3 mL). Na₂CO₃ (953.67 mg, 9.000 mmol, 6 equiv) was added at room temperature. The resulting mixture was stirred at 50° C. overnight. The resulting mixture was concentrated under reduced pressure. The resulting mixture was extracted with DCM. The combined organic layers were washed with water and dried over anhydrous Na₂SO₄. After filtration, the filtrate was concentrated under reduced pressure. The residue was purified by silica gel column chromatography eluted with CH₂Cl₂:MeOH (5:1) to afford tert-butyl 4-(4-{5-bromo-4-[2-fluoro-3-(propane-1-sulfonamido)phenyl]-1,3-thiazol-2-yl}phenyl)piperazine-1-carboxylate (900 mg, 93.83%) as a yellow solid. LCMS: C₂₇H₃₂BrFN₄O₄S₂ requires: 638.1, found: m/z=639.1 [M+H]⁺.

### Step-5: Synthesis of tert-butyl 4-(4-{4-[2-fluoro-3-(propane-1-sulfonamido)phenyl]-5-(pyridin-4-yl)-1,3-thiazol-2-yl}phenyl)piperazine-1-carboxylate

To a mixture of tert-butyl 4-(4-{5-bromo-4-[2-fluoro-3-(propane-1-sulfonamido)phenyl]-1,3-thiazol-2-yl}phenyl)piperazine-1-carboxylate (900 mg, 1.407 mmol, 1 equiv) and pyridin-4-ylboronic acid (259.45 mg, 2.111 mmol, 1.5 equiv) in dioxane (10 mL) and H₂O (1 mL) was added Pd(dppf)Cl₂·CH₂Cl₂ (229.26 mg, 0.281 mmol, 0.2 equiv) in portions at room temperature. The resulting mixture was stirred for 2 h at 90° C. under a nitrogen atmosphere. The resulting mixture was concentrated under reduced pressure. The residue was purified by silica gel column chromatography eluted with DCM:EtOAc (5:1) to afford tert-butyl 4-(4-{4-[2-fluoro-3-(propane-1-sulfonamido)phenyl]-5-(pyridin-4-yl)-1,3-thiazol-2-yl}phenyl)piperazine-1-carboxylate (200 mg, 22.28%) as a yellow solid. LCMS: C₃₂H₃₆FN₅O₄S₂ requires: 637.2, found: m/z=638.2 [M+H]⁺.

### Step-6: Synthesis of N-(2-fluoro-3-{2-[4-(piperazin-1-yl)phenyl]-5-(pyridin-4-yl)-1,3-thiazol-4-yl}phenyl)propane-1-sulfonamide Hydrochloride

To a mixture of tert-butyl 4-(4-{4-[2-fluoro-3-(propane-1-sulfonamido)phenyl]-5-(pyridin-4-yl)-1,3-thiazol-2-yl}phenyl)piperazine-1-carboxylate (200 mg, 0.313 mmol, 1 equiv) in DCM (2 mL) was added HCl (gas) in 1,4-dioxane (2 mL, 4 M). The resulting mixture was stirred at room temperature overnight. The resulting mixture was concentrated under reduced pressure. The residue was purified by reversed-phase flash chromatography with the following conditions: column, C18 silica gel; mobile phase, MeCN in water (0.5% HCl), 10% to 50% gradient in 10 min; detector, UV 254 nm to afford N-(2-fluoro-3-{2-[4-(piperazin-1-yl)phenyl]-5-(pyridin-4-yl)-1,3-thiazol-4-yl}phenyl)propane-1-sulfonamide hydrochloride salt (136.9 mg, 80.90%) as a yellow solid. LCMS: C₂₇H₂₈FN₅O₂S₂ requires: 537.2, found: m/z=538.2 [M+H]⁺.

¹H NMR (400 MHz, CD₃OD) δ 8.97 (d, J=6.2 Hz, 2H), 8.56 (d, J=6.2 Hz, 2H), 8.31-8.16 (m, 2H), 8.13-8.08 (m, 1H), 7.96 (d, J=2.6 Hz, 1H), 7.65-7.52 (m, 2H), 7.28-7.25 (m, 1H), 3.32-3.26 (m, 8H), 3.21-3.13 (m, 2H), 1.97-1.86 (m, 2H), 1.09-1.05 (m, 3H).

### Synthesis of N-{3-[2-tert-butyl-5-(2-{[4-(piperazin-1-yl)phenyl]amino}pyrimidin-4-yl)-1,3-thiazol-4-yl]-2-fluorophenyl}propane-1-sulfonamide Hydrochloride Salt

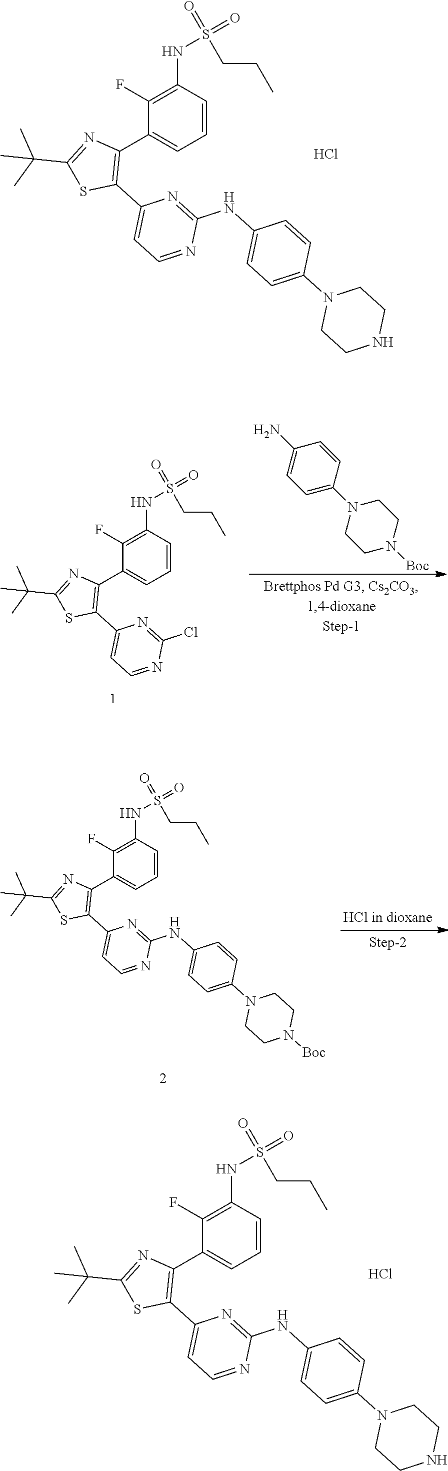

### Step-1: Synthesis of tert-butyl 4-{4-[(4-{2-tert-butyl-4-[2-fluoro-3-(propane-1-sulfonamido)phenyl]-1,3-thiazol-5-yl}pyrimidin-2-yl)amino]phenyl}piperazine-1-carboxylate (2)

To a mixture of N-{3-[2-tert-butyl-5-(2-chloropyrimidin-4-yl)-1,3-thiazol-4-yl]-2-fluorophenyl}propane-1-sulfonamide (800 mg, 1.70 mmol, 1 equiv) in dioxane (10 mL) was added tert-butyl 4-(4-aminophenyl)piperazine-1-carboxylate (1.18 g, 4.265 mmol, 2.5 equiv), BrettPhosPdG₃ (154 mg, 0.17 mmol, 0.1 equiv), and Cs₂CO₃ (1.11 g, 3.41 mmol, 2 equiv) at room temperature. The resulting mixture was stirred 2 h at 80° C. under a nitrogen atmosphere. The resulting mixture was concentrated under reduced pressure. The residue was purified by reverse phase flash chromatography with H₂O:MeCN (1:8) to afford tert-butyl 4-{4-[(4-{2-tert-butyl-4-[2-fluoro-3-(propane-1-sulfonamido)phenyl]-1,3-thiazol-5-yl}pyrimidin-2-yl)amino]phenyl}piperazine-1-carboxylate (500 mg, 39.23%) as a yellow solid. LCMS: C₃₅H₄₄FN₇O₄S₂ requires: 709.3, found: m/z=710.2 [M+H]⁺.

### Step-2: Synthesis of N-{3-[2-tert-butyl-5-(2-{[4-(piperazin-1-yl)phenyl]amino}pyrimidin-4-yl)-1,3-thiazol-4-yl]-2-fluorophenyl}propane-1-sulfonamide Hydrochloride

A mixture of tert-butyl 4-{4-[(4-{2-tert-butyl-4-[2-fluoro-3-(propane-1-sulfonamido)phenyl]-1,3-thiazol-5-yl}pyrimidin-2-yl)amino]phenyl}piperazine-1-carboxylate (500 mg, 0.70 mmol, 1 equiv) in HCl/1,4-dioxane (10 mL, 4 M) was stirred overnight at room temperature. The resulting mixture was concentrated under reduced pressure to afford N-{3-[2-tert-butyl-5-(2-{[4-(piperazin-1-yl)phenyl]amino}pyrimidin-4-yl)-1,3-thiazol-4-yl]-2-fluorophenyl}propane-1-sulfonamide hydrochloride salt (371.7 mg, crude) as a brown solid. LCMS: C₃₀H₃₆FN₇O₂S₂ requires: 609.2, found: m/z=610.2 [M+H]⁺.

¹H NMR (400 MHz, CD₃OD) δ 8.11 (s, 1H), 7.62-7.45 (m, 2H), 7.36-7.32 (m, 3H), 7.14-7.12 (m, 2H), 6.78 (s, 1H), 3.59-3.41 (m, 8H), 3.09-3.06 (m, 2H), 1.84-1.79 (m, 2H), 1.52 (s, 9H), 1.07-1.03 (m, 3H).

### Synthesis of N-(2-fluoro-3-{1-[4-(piperazine-1-carbonyl)phenyl]-3-(pyridin-4-yl)pyrazol-4-yl}phenyl)propane-1-sulfonamide Hydrochloride Salt

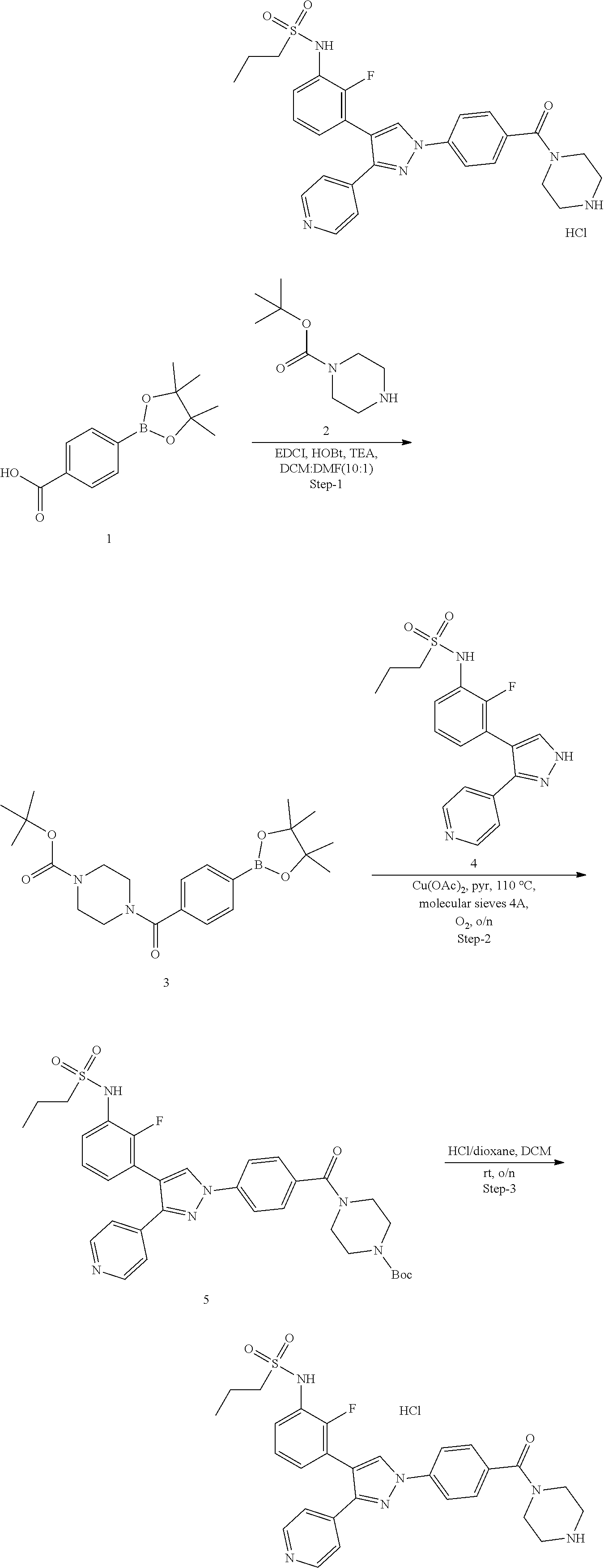

### Step-1: Synthesis of tert-butyl 4-[4-(4,4,5,5-tetramethyl-1,3,2-dioxaborolan-2-yl)benzoyl]piperazine-1-carboxylate (3)

To a mixture of 4-(4,4,5,5-tetramethyl-1,3,2-dioxaborolan-2-yl)benzoic acid (3.2 g, 12.886 mmol, 1.2 equiv) in DCM (20 mL) and DMF (2 mL) was added HOBT (1.74 g, 12.886 mmol, 1.2 equiv) and tert-butyl piperazine-1-carboxylate (2.0 g, 10.738 mmol, 1.00 equiv) at room temperature. To the above mixture was added EDCI (2.47 g, 12.886 mmol, 1.2 equiv) at room temperature. The resulting mixture was stirred for 2 h at room temperature. The resulting mixture was extracted with CH₂Cl₂. The combined organic layers were washed with water and dried over anhydrous Na₂SO₄. After filtration, the filtrate was concentrated under reduced pressure. The residue was purified by silica gel column chromatography eluted with PE:EtOAc (2:1) to afford tert-butyl 4-[4-(4,4,5,5-tetramethyl-1,3,2-dioxaborolan-2-yl)benzoyl]piperazine-1-carboxylate (2.2 g, 49.21%) as a white solid. LCMS: C₂₂H₃₃BN₂O₅ requires: 416.2, found: m/z=417.2 [M+H]⁺.

### Step-2: Synthesis of tert-butyl 4-(4-{4-[2-fluoro-3-(propane-1-sulfonamido)phenyl]-3-(pyridin-4-yl)pyrazol-1-yl}benzoyl)piperazine-1-carboxylate (5)

To a stirred mixture of tert-butyl 4-[4-(4,4,5,5-tetramethyl-1,3,2-dioxaborolan-2-yl)benzoyl]piperazine-1-carboxylate (1.50 g, 3.603 mmol, 1.5 equiv) and N-{2-fluoro-3-[3-(pyridin-4-yl)-1H-pyrazol-4-yl]phenyl}propane-1-sulfonamide (0.58 g, 1.609 mmol, 0.67 equiv) in pyridine (15 mL) was added Cu(OAc)₂ (0.87 g, 4.804 mmol, 2.0 equiv) and molecular sieves 4 A (0.58 g) at room temperature. The resulting mixture was stirred at 110° C. overnight under an oxygen atmosphere. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with CH₂Cl₂:MeOH (10:1) to afford tert-butyl 4-(4-{4-[2-fluoro-3-(propane-1-sulfonamido)phenyl]-3-(pyridin-4-yl)pyrazol-1-yl}benzoyl)piperazine-1-carboxylate (400 mg, 25.67%) as a yellow solid. LCMS: C₃₃H₃₇FN₆O₅S requires: 648.3, found: m/z=649.3 [M+H]⁺.

### Step-3: Synthesis of N-(2-fluoro-3-{1-[4-(piperazine-1-carbonyl)phenyl]-3-(pyridin-4-yl)pyrazol-4-yl}phenyl)propane-1-sulfonamide Hydrochloride

To a mixture of tert-butyl 4-(4-{4-[2-fluoro-3-(propane-1-sulfonamido)phenyl]-3-(pyridin-4-yl)pyrazol-1-yl}benzoyl)piperazine-1-carboxylate (400 mg, 0.617 mmol, 1 equiv) in DCM (2 mL) was added HCl in 1,4-dioxane (5 mL, 4 M). The resulting mixture was stirred at room temperature overnight. The resulting mixture was concentrated under reduced pressure. The residue was purified by reverse-phase flash chromatography with the following conditions: column, C18 silica gel; mobile phase, MeCN in water (0.5% HCl), 10% to 50% gradient in 10 min; detector, UV 254 nm to afford N-(2-fluoro-3-f{1-[4-(piperazine-1-carbonyl)phenyl]-3-(pyridin-4-yl)pyrazol-4-yl}phenyl)propane-1-sulfonamide hydrochloride salt (216.3 mg, 57.98%) as a white solid. LCMS: C₂₈H₂₉FN₆O₃S requires: 548.2, found: m/z=549.3 [M+H]⁺.

¹H NMR (400 MHz, DMSO-d₆) δ 9.74 (s, 1H), 9.48-9.44 (m, 2H), 9.07-9.02 (m, 1H), 8.85-8.80 (m, 1H), 8.15-8.09 (m, 2H), 7.93-7.89 (m, 2H), 7.75-7.68 (m, 2H), 7.57-7.48 (m, 1H), 7.46-7.38 (m, 1H), 7.38-7.30 (m, 1H), 3.78-3.41 (m, 4H), 3.21-3.16 (m, 4H), 3.11-3.03 (m, 2H), 1.78-1.63 (m, 2H), 0.98-0.89 (m, 3H).

### Synthesis of N-(2-fluoro-3-{1-[4-(piperidin-4-yloxy)phenyl]-3-(pyridin-4-yl)pyrazol-4-yl}phenyl)propane-1-sulfonamide Hydrochloride Salt

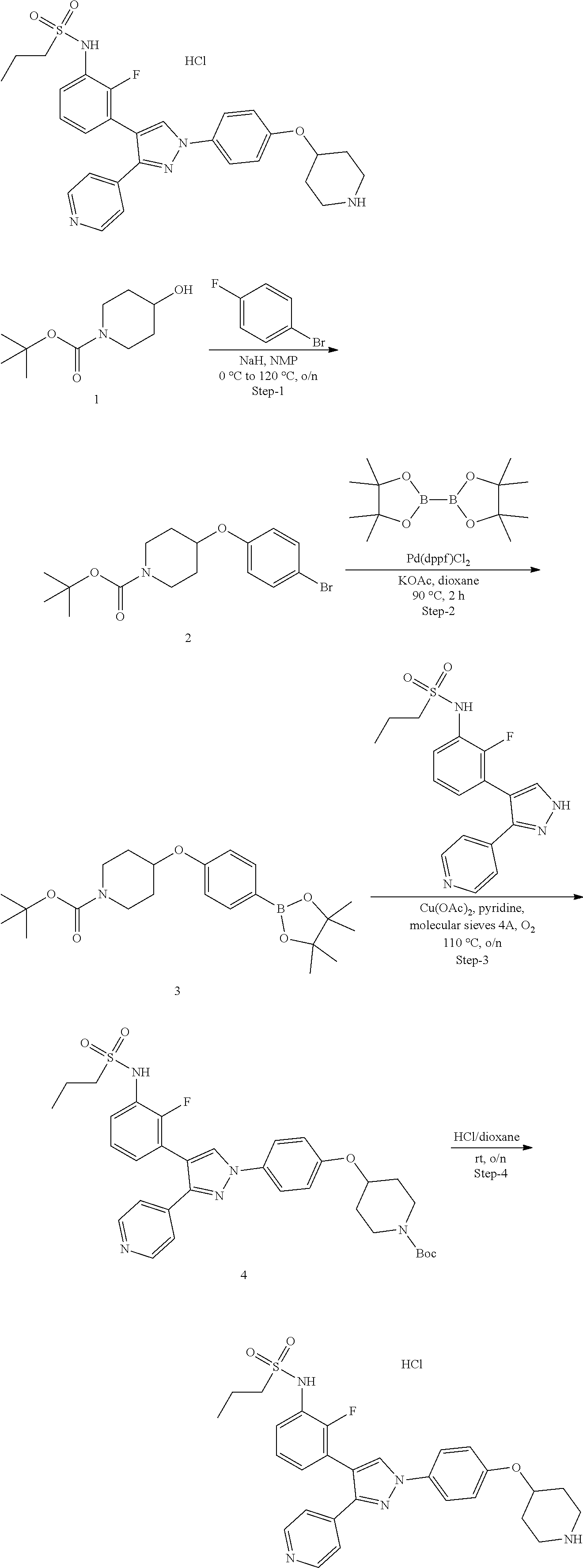

### Step-1: Synthesis of tert-butyl 4-(4-bromophenoxy)piperidine-1-carboxylate (2)

To a mixture of NaH (238.47 mg, 9.93 mmol, 1 equiv) in NMP (20 mL) was added tert-butyl 4-hydroxypiperidine-1-carboxylate (2 g, 9.93 mmol, 1 equiv) at 0° C. The resulting mixture was stirred one hour at 0° C. under a nitrogen atmosphere. To the above mixture was added 4-bromofluorobenzene (1.74 g, 9.93 mmol, 1 equiv) at 0° C. The resulting mixture was stirred overnight at 120° C. under the nitrogen atmosphere. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with PE:EtOAc (4:1) to afford tert-butyl 4-(4-bromophenoxy)piperidine-1-carboxylate (1.3 g, 33.05%) as a white solid. LCMS: C₁₆H₂₂BrNO₃ requires: 355.1, found: m/z=356.1 [M+H]⁺.

### Step-2: Synthesis of tert-butyl 4-[4-(4,4,5,5-tetramethyl-1,3,2-dioxaborolan-2-yl)phenoxy]piperidine-1-carboxylate (3)

To a mixture of tert-butyl 4-(4-bromophenoxy)piperidine-1-carboxylate (1.3 g, 3.64 mmol, 1 equiv) in dioxane (10 mL) was added 4,4,5,5-tetramethyl-2-(tetramethyl-1,3,2-dioxaborolan-2-yl)-1,3,2-dioxaborolane (1.85 g, 7.29 mmol, 2 equiv), KOAc (1.07 g, 10.94 mmol, 3 equiv), and Pd(dppf)Cl₂ (1.34 g, 1.85 mmol, 0.5 equiv) at room temperature under a nitrogen atmosphere. The resulting mixture was stirred 2 h at 90° C. The resulting mixture was concentrated under reduced pressure. The residue was purified by silica gel column chromatography eluted with PE:EtOAc (2:1) to afford tert-butyl 4-[4-(4,4,5,5-tetramethyl-1,3,2-dioxaborolan-2-yl)phenoxy]piperidine-1-carboxylate (1 g, 61.15%) as a yellow solid. LCMS: C₂₂H₃₄BNO₅ requires: 403.3, found: m/z=404.2 [M+H]⁺.

### Step-3: Synthesis of tert-butyl 4-(4-{4-[2-fluoro-3-(propane-1-sulfonamido)phenyl]-3-(pyridin-4-yl)pyrazol-1-yl}phenoxy)piperidine-1-carboxylate (4)

To a mixture of tert-butyl 4-[4-(4,4,5,5-tetramethyl-1,3,2-dioxaborolan-2-yl)phenoxy]piperidine-1-carboxylate (1 g, 2.47 mmol, 1 equiv) in pyridine (10 mL) was added N-{2-fluoro-3-[3-(pyridin-4-yl)-1H-pyrazol-4-yl]phenyl}propane-1-sulfonamide (893 mg, 2.47 mmol, 1 equiv), Cu(OAc)₂ (900 mg, 4.95 mmol, 2 equiv), and molecular sieves 4 A (893 mg) at room temperature. The resulting mixture was stirred at 110° C. overnight under an oxygen atmosphere. The resulting mixture was concentrated under reduced pressure. The residue was purified by reverse phase flash chromatography with the following conditions H₂O:MeCN (1:1) to afford tert-butyl 4-(4-{4-[2-fluoro-3-(propane-1-sulfonamido)phenyl]-3-(pyridin-4-yl)pyrazol-1-yl}phenoxy)piperidine-1-carboxylate (400 mg, 24.11%) as a white solid. LCMS: C₃₃H₃₈FN₅O₅S requires: 635.3, found: m/z=636.2 [M+H]⁺.

### Step-4: Synthesis of N-(2-fluoro-3-{1-[4-(piperidin-4-yloxy)phenyl]-3-(pyridin-4-yl)pyrazol-4-yl}phenyl)propane-1-sulfonamide Hydrochloride

A mixture of tert-butyl 4-(4-{4-[2-fluoro-3-(propane-1-sulfonamido)phenyl]-3-(pyridin-4-yl)pyrazol-1-yl}phenoxy)piperidine-1-carboxylate (500 mg, 0.78 mmol, 1 equiv) in HCl/1,4-dioxane (10 mL, 4 M) was stirred overnight at room temperature. The resulting mixture was concentrated under reduced pressure to afford N-(2-fluoro-3-{1-[4-(piperidin-4-yloxy)phenyl]-3-(pyridin-4-yl)pyrazol-4-yl}phenyl)propane-1-sulfonamide hydrochloride salt (341.3 mg, crude) as a yellow solid. LCMS: C₂₈H₃₀FN₅O₃S requires: 535.2, found: m/z=536.2 [M+H]⁺.

¹H NMR (400 MHz, CD₃OD) δ 8.75-8.73 (m, 2H), 8.58 (s, 1H), 8.21-8.20 (m, 2H), 7.93-7.91 (m, 2H), 7.58-7.56 (m, 1H), 7.38-7.30 (m, 2H), 7.23-7.20 (m, 2H), 3.47-3.40 (m, 2H), 3.23-3.26 (m, 2H), 3.14-3.11 (m, 2H), 2.26-2.20 (m, 2H), 2.10-2.05 (m, 2H), 1.93 (s, 1H), 1.87-1.81 (m, 2H), 1.05-1.01 (m, 3H).

### Synthesis of N-{2-fluoro-3-[1-(piperidin-4-yl)-3-(pyridin-4-yl)pyrazol-4-yl]phenyl}propane-1-sulfonamide Hydrochloride Salt

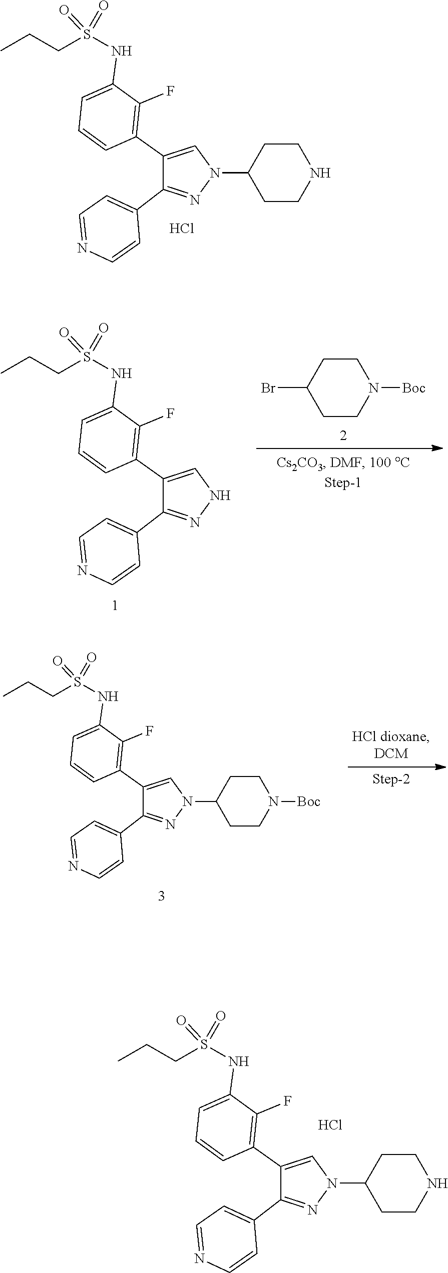

### Step-1: Synthesis of tert-butyl 4-{4-[2-fluoro-3-(propane-1-sulfonamido)phenyl]-3-(pyridin-4-yl)pyrazol-1-yl}piperidine-1-carboxylate (3)

To a stirred mixture of N-{2-fluoro-3-[3-(pyridin-4-yl)-1H-pyrazol-4-yl]phenyl}propane-1-sulfonamide (400 mg, 1.110 mmol, 1 equiv) and tert-butyl 4-bromopiperidine-1-carboxylate (322.50 mg, 1.221 mmol, 1.1 equiv) in DMF (4 mL) was added Cs₂CO₃ (723.22 mg, 2.220 mmol, 2.0 equiv) at room temperature. The resulting mixture was stirred at 100° C. overnight. The resulting mixture was extracted with EtOAc. The combined organic layers were washed with water and dried over anhydrous Na₂SO₄. The resulting mixture was concentrated under reduced pressure. The residue was purified by silica gel column chromatography eluted with CH₂Cl₂:MeOH (10:1) to afford tert-butyl 4-{4-[2-fluoro-3-(propane-1-sulfonamido)phenyl]-3-(pyridin-4-yl)pyrazol-1-yl}piperidine-1-carboxylate (220 mg, 36.46%) as a yellow solid. LCMS: C₂₇H₃₄FN₅O₄S requires: 543.2, found: m/z=544.2 [M+H]⁺.

### Step-2: Synthesis of N-{2-fluoro-3-[1-(piperidin-4-yl)-3-(pyridin-4-yl)pyrazol-4-yl]phenyl}propane-1-sulfonamide Hydrochloride

To a mixture of tert-butyl 4-{4-[2-fluoro-3-(propane-1-sulfonamido)phenyl]-3-(pyridin-4-yl)pyrazol-1-yl}piperidine-1-carboxylate (200 mg, 0.368 mmol, 1 equiv) in DCM (2 mL) was added HCl (gas) in 1,4-dioxane (2 mL, 4 M). The resulting mixture was stirred at room temperature overnight. The resulting mixture was concentrated under reduced pressure. The residue was purified by reversed-phase flash chromatography with the following conditions: column, C18 silica gel; mobile phase, MeCN in Water (0.5% HCl), 10% to 50% gradient in 10 min; detector, UV 254 nm to afford N-{2-fluoro-3-[1-(piperidin-4-yl)-3-(pyridin-4-yl)pyrazol-4-yl]phenyl}propane-1-sulfonamide hydrochloride salt (140 mg, 79.28%) as a yellow solid. LCMS: C₂₂H₂₆FN₅O₂S requires: 443.2, found: m/z=444.1 [M+H]⁺.

¹H NMR (400 MHz, CD₃OD) δ 8.78-8.67 (m, 2H), 8.19-8.11 (m, 3H), 7.63-7.52 (m, 1H), 7.39-7.29 (m, 2H), 4.85-4.79 (m, 1H), 3.73-3.62 (m, 2H), 3.39-3.36 (m, 1H), 3.35-3.29 (m, 1H), 3.21-3.11 (m, 2H), 2.55-2.22 (m, 4H), 1.91-1.79 (m, 2H), 1.10-0.95 (m, 3H).

### Synthesis of N-{2-fluoro-3-[3-(morpholin-4-yl)-1-(piperidin-4-yl)pyrazol-4-yl]phenyl}propane-1-sulfonamide; Trifluoroacetic Acid Salt

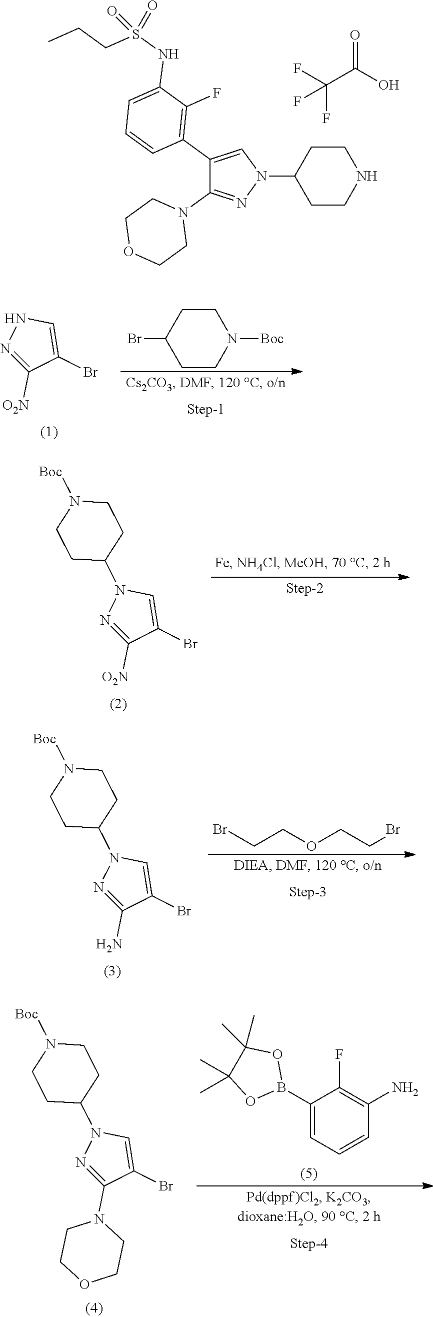

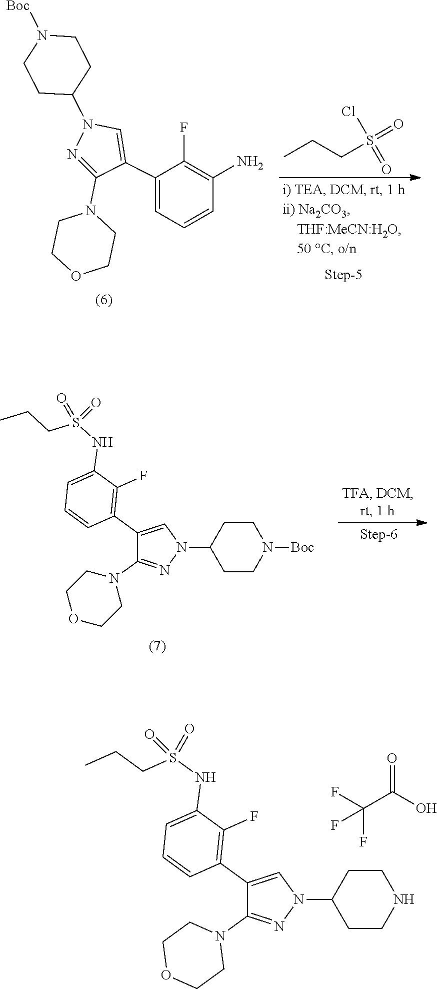

### Step-1: Synthesis of tert-butyl 4-(4-bromo-3-nitropyrazol-1-yl)piperidine-1-carboxylate (2)

To a mixture of 4-bromo-3-nitro-1H-pyrazole (2 g, 10.418 mmol, 1 equiv) in DMF (20 mL) was added tert-butyl 4-bromopiperidine-1-carboxylate (3.03 g, 11.460 mmol, 1.1 equiv) and Cs₂CO₃ (3.39 g, 10.418 mmol, 1 equiv) at room temperature. The resulting mixture was stirred at 120° C. overnight. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with PE:EtOAc (20:80) to afford tert-butyl 4-(4-bromo-3-nitropyrazol-1-yl)piperidine-1-carboxylate (3 g, 76.74%) as a yellow solid. LCMS: C₁₃H₁₉BrN₄O₄ requires: 374.1, found: m/z=375.1 [M+H]⁺.

### Step-2: Synthesis of tert-butyl 4-(3-amino-4-bromopyrazol-1-yl)piperidine-1-carboxylate (3)

To a mixture of tert-butyl 4-(4-bromo-3-nitropyrazol-1-yl)piperidine-1-carboxylate (3 g, 7.995 mmol, 1 equiv) and Fe (22.32 mg, 0.400 mmol, 5 equiv) in MeOH (30 mL) was added saturated aqueous NH₄Cl (4 mL) at room temperature. The resulting mixture was stirred for 2 h at 70° C. The resulting mixture was concentrated under reduced pressure. The residue was purified by silica gel column chromatography eluted with CH₂Cl₂:MeOH (10:1) to afford tert-butyl 4-(3-amino-4-bromopyrazol-1-yl)piperidine-1-carboxylate (2.6 g, 94.19%) as a yellow solid. LCMS: C₁₃H₂₁BrN₄O₂ requires: 344.1, found: m/z=345.1 [M+H]⁺.

### Step-3: Synthesis of tert-butyl 4-[4-bromo-3-(morpholin-4-yl)pyrazol-1-yl]piperidine-1-carboxylate (4)

To a mixture of tert-butyl 4-(3-amino-4-bromopyrazol-1-yl)piperidine-1-carboxylate (2.5 g, 7.241 mmol, 1 equiv) and DIEA (2.81 g, 21.723 mmol, 3 equiv) in DMF (25 mL) was added 1-bromo-2-(2-bromoethoxy)ethane (2.02 g, 8.689 mmol, 1.2 equiv) at room temperature. The resulting mixture was stirred overnight at 120° C. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with CH₂Cl₂:EtOAc (75:25) to afford tert-butyl 4-[4-bromo-3-(morpholin-4-yl)pyrazol-1-yl]piperidine-1-carboxylate (1.4 g, 46.55%) as a yellow oil. LCMS: C₁₇H₁₆ClFN₄O₂S₂ requires: 414.1, found: m/z=414.2 [M+H]⁺.

### Step-4: Synthesis of tert-butyl 4-[4-(3-amino-2-fluorophenyl)-3-(morpholin-4-yl)pyrazol-1-yl]piperidine-1-carboxylate (6)

To a mixture of tert-butyl 4-[4-bromo-3-(morpholin-4-yl)pyrazol-1-yl]piperidine-1-carboxylate (1.4 g, 3.371 mmol, 1 equiv) and 2-fluoro-3-(4,4,5,5-tetramethyl-1,3,2-dioxaborolan-2-yl) aniline (1.04 g, 4.382 mmol, 1.3 equiv) in dioxane (15 mL) and H₂O (1.5 mL) was added Pd(dppf)Cl₂·CH₂Cl₂ (274.59 mg, 0.337 mmol, 0.1 equiv) and K₂CO₃ (931.7 mg, 6.742 mmol, 2 equiv) at room temperature under a nitrogen atmosphere. The resulting mixture was stirred for 2 h at 90° C. under the nitrogen atmosphere. The resulting mixture was concentrated under vacuum. The residue was purified by silica gel column chromatography eluted with CH₂Cl₂:MeOH (10:1) to afford tert-butyl 4-[4-(3-amino-2-fluorophenyl)-3-(morpholin-4-yl)pyrazol-1-yl]piperidine-1-carboxylate (1 g, 66.59%) as a dark yellow oil. LCMS: C₂₃H₃₂FN₅O₃ requires: 445.3, found: m/z=446.2 [M+H]⁺.

### Step-5: Synthesis of tert-butyl 4-{4-[2-fluoro-3-(propane-1-sulfonamido)phenyl]-3-(morpholin-4-yl)pyrazol-1-yl}piperidine-1-carboxylate (7)

To a mixture of tert-butyl 4-[4-(3-amino-2-fluorophenyl)-3-(morpholin-4-yl)pyrazol-1-yl]piperidine-1-carboxylate (1 g, 2.244 mmol, 1 equiv) and TEA (1.14 g, 11.220 mmol, 5 equiv) in DCM (10 mL) was added propane-1-sulfonyl chloride (384.07 mg, 2.693 mmol, 1.2 equiv) at 0° C. The resulting mixture was stirred for one hour at room temperature. The resulting mixture was concentrated under vacuum. To the above mixture was added Na₂CO₃ (0.71 g, 6.732 mmol, 3 equiv) in THF (10 mL), MeCN (10 mL), and H₂O (10 mL) at room temperature. The resulting mixture was stirred overnight at 50° C. The resulting mixture was extracted with CH₂Cl₂. The combined organic layers were washed with water and dried over anhydrous Na₂SO₄. After filtration, the filtrate was concentrated under reduced pressure. The residue was purified by silica gel column chromatography eluted with CH₂Cl₂:MeOH (10:1). The residue product was purified by reverse phase flash chromatography with MeCN:H₂O (60:40) to afford tert-butyl 4-{4-[2-fluoro-3-(propane-1-sulfonamido)phenyl]-3-(morpholin-4-yl)pyrazol-1-yl}piperidine-1-carboxylate (600 mg, 48.46%) as a yellow solid. LCMS: C₂₆H₃₈FN₅O₅S requires: 551.3, found: m/z=552.3 [M+H]⁺. ¹H NMR (300 MHz, CD₃OD) δ 7.75 (d, J=2.4 Hz, 1H), 7.70-7.61 (m, 1H), 7.40-7.34 (m, 1H), 7.21-7.12 (m, 1H), 4.33-4.16 (m, 3H), 3.77-3.72 (m, 4H), 3.19-3.09 (m, 2H), 3.06-2.90 (m, 6H), 2.15-2.03 (m, 2H), 1.97-1.83 (m, 4H), 1.50 (s, 9H), 1.08-1.03 (m, 3H).

### Step-6: Synthesis of N-{2-fluoro-3-[3-(morpholin-4-yl)-1-(piperidin-4-yl)pyrazol-4-yl]phenyl}propane-1-sulfonamide, Trifluoroacetic Acid

To a mixture of tert-butyl 4-{4-[2-fluoro-3-(propane-1-sulfonamido)phenyl]-3-(morpholin-4-yl)pyrazol-1-yl}piperidine-1-carboxylate (600 mg, 1.088 mmol, 1 equiv) in DCM (9 mL) was added TFA (3 mL) at room temperature. The resulting mixture was stirred for one hour at room temperature. The resulting mixture was concentrated under vacuum. This resulted in N-{2-fluoro-3-[3-(morpholin-4-yl)-1-(piperidin-4-yl)pyrazol-4-yl]phenyl}propane-1-sulfonamide, trifluoroacetic acid salt (232.0 mg, crude) as a yellow solid. LCMS: C₂₁H₃₀FN₅O₃S requires: 451.2, found: m/z=452.1 [M+H]⁺.

¹H NMR (300 MHz, CD₃OD) δ 7.77 (d, J=2.4 Hz, 1H), 7.69-7.63 (m, 1H), 7.40-7.35 (m, 1H), 7.19-7.14 (m, 1H), 4.51-4.38 (m, 1H), 3.77-3.72 (m, 4H), 3.62-3.56 (m, 2H), 3.31-3.10 (m, 4H), 3.02-2.99 (m, 4H), 2.32-2.27 (m, 4H), 1.91-1.84 (m, 2H), 1.08-1.03 (m, 3H).

### Synthesis of N-{3-[5-(2-aminopyrimidin-4-yl)-2-[4-(piperazin-1-yl)phenyl]-1,3-thiazol-4-yl]-2-fluorophenyl}propane-1-sulfonamide Hydrochloride Salt

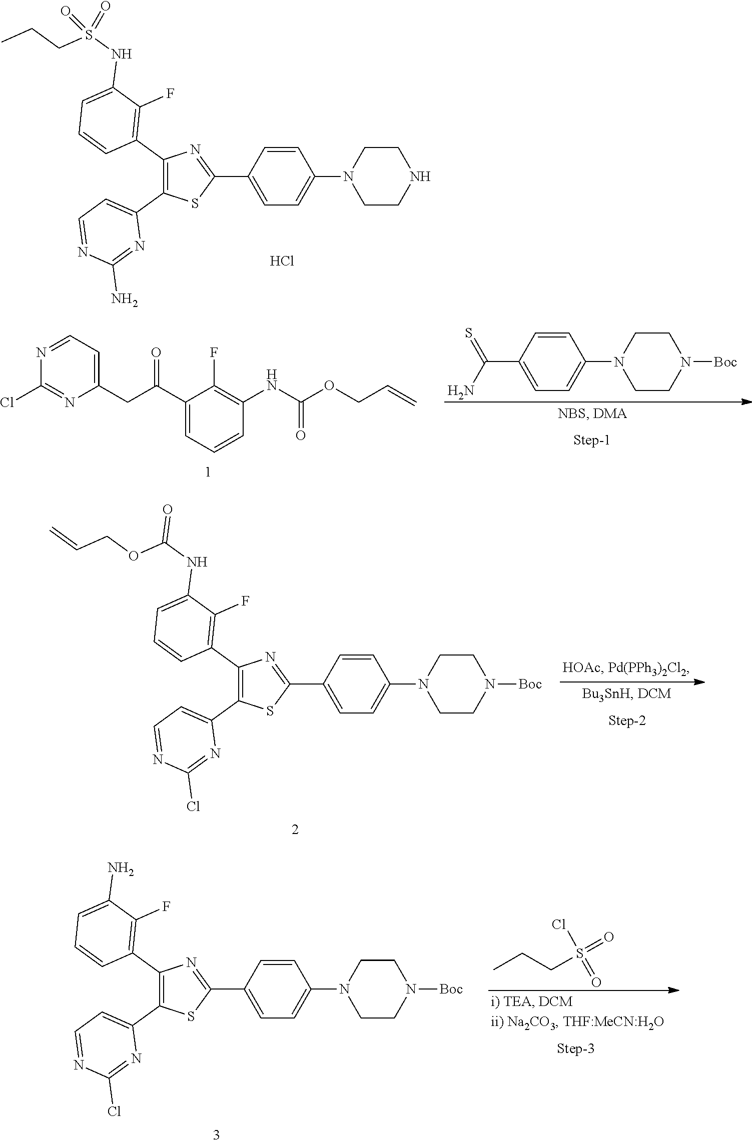

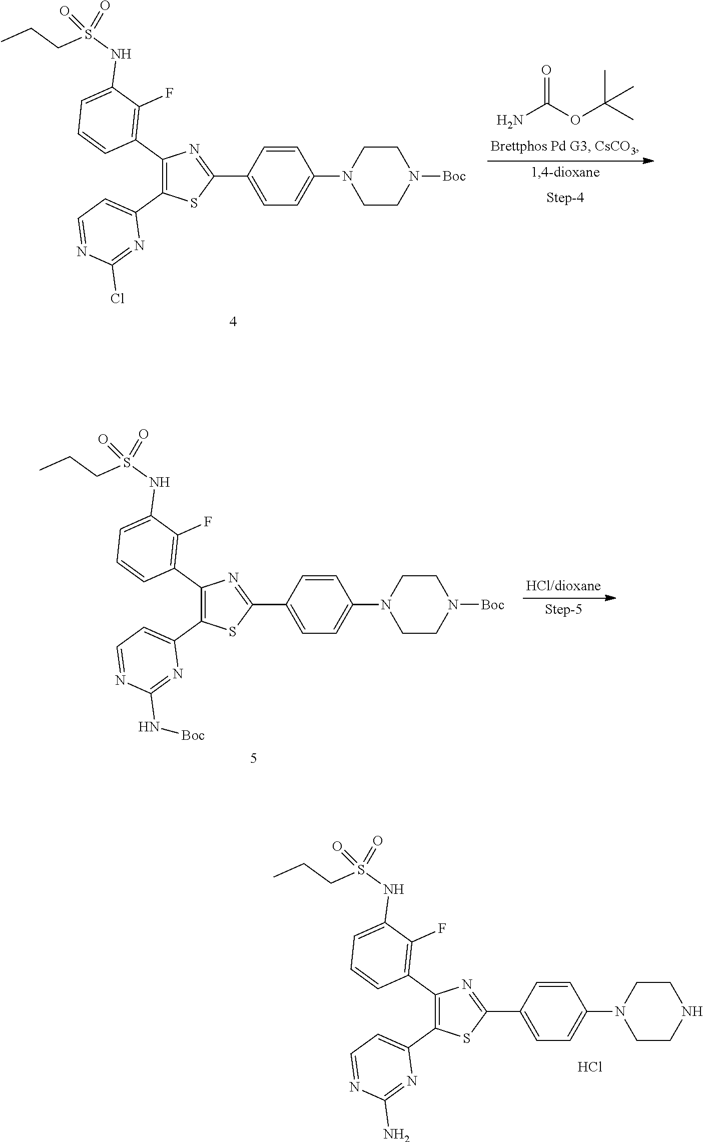

### Step-1: Synthesis of tert-butyl 4-{4-[5-(2-chloropyrimidin-4-yl)-4-(2-fluoro-3-{[(prop-2-en-1-yloxy)carbonyl]amino}phenyl)-1,3-thiazol-2-yl]phenyl}piperazine-1-carboxylate (2)

To a mixture of prop-2-en-1-yl N-{3-[2-(2-chloropyrimidin-4-yl)acetyl]-2-fluorophenyl}carbamate (2 g, 5.71 mmol, 1 equiv) in DMA (20 mL) was added tert-butyl 4-(4-carbamothioylphenyl)piperazine-1-carboxylate (2.21 g, 6.86 mmol, 1.2 equiv) and NBS (508 mg, 2.85 mmol, 0.5 equiv) at 0° C. The resulting mixture was stirred 2 h at 60° C. The resulting mixture was extracted with EtOAc. The combined organic layers were washed with water and dried over anhydrous Na₂SO₄. After filtration, the filtrate was concentrated under reduced pressure. The residue was purified by silica gel column chromatography, eluted with PE:EtOAc (1:1) to afford tert-butyl 4-{4-[5-(2-chloropyrimidin-4-yl)-4-(2-fluoro-3-{[(prop-2-en-1-yloxy)carbonyl]amino}phenyl)-1,3-thiazol-2-yl]phenyl}piperazine-1-carboxylate (1.5 g, 36.26%) as a yellow green solid. LCMS: C₃₂H₃₂ClFN₆O₄S requires: 650.2, found: m/z=651.1 [M+H]⁺.

### Step-2: Synthesis of tert-butyl 4-{4-[4-(3-amino-2-fluorophenyl)-5-(2-chloropyrimidin-4-yl)-1,3-thiazol-2-yl]phenyl}piperazine-1-carboxylate (3)

To a mixture of tert-butyl 4-{4-[5-(2-chloropyrimidin-4-yl)-4-(2-fluoro-3-{[(prop-2-en-1-yloxy)carbonyl]amino}phenyl)-1,3-thiazol-2-yl]phenyl}piperazine-1-carboxylate (1.5 g, 2.30 mmol, 1 equiv), HOAc (332 mg, 5.53 mmol, 2.4 equiv), and Pd(PPh₃)₂Cl₂ (32 mg, 0.046 mmol, 0.02 equiv) in DCM (15 mL) was added n-Bu₃SnH (1 g, 3.45 mmol, 1.5 equiv) at 0° C. under a nitrogen atmosphere. The resulting mixture was stirred for 30 min at room temperature. The reaction was quenched by the addition of saturated aqueous NaHCO₃ (10 mL) at 0° C. The resulting mixture was extracted with DCM. The combined organic layers were washed with water and dried over anhydrous Na₂SO₄. After filtration, the filtrate was concentrated under reduced pressure. The residue was purified by silica gel column chromatography eluted with PE:EtOAc (2:1) to afford tert-butyl 4-{4-[4-(3-amino-2-fluorophenyl)-5-(2-chloropyrimidin-4-yl)-1,3-thiazol-2-yl]phenyl}piperazine-1-carboxylate (1.2 g, 83.59%) as a yellow solid. LCMS: C₂₈H₂₈ClFN₆O₂S requires: 566.2, found: m/z=567.2 [M+H]⁺.

### Step-3: Synthesis of tert-butyl 4-{4-[5-(2-chloropyrimidin-4-yl)-4-[2-fluoro-3-(propane-1-sulfonamido)phenyl]-1,3-thiazol-2-yl]phenyl}piperazine-1-carboxylate (4)

To a mixture of tert-butyl 4-{4-[4-(3-amino-2-fluorophenyl)-5-(2-chloropyrimidin-4-yl)-1,3-thiazol-2-yl]phenyl}piperazine-1-carboxylate (1.2 g, 2.11 mmol, 1 equiv) and TEA (642 mg, 6.34 mmol, 3 equiv) in DCM (10 mL) was added propane-1-sulfonyl chloride (452 mg, 3.17 mmol, 1.5 equiv) at 0° C. The resulting mixture was stirred for one hour at room temperature. The resulting mixture was concentrated under reduced pressure. The crude product was dissolved in THF (10 mL), MeCN (10 mL), and H₂O (10 mL). Na₂CO₃ (2 g, 19.25 mmol, 10 equiv) was added at room temperature. The resulting mixture was stirred overnight at 50° C. The resulting mixture was concentrated under vacuum. The residue was extracted with DCM. The combined organic layers were washed with water and dried over anhydrous Na₂SO₄. After filtration, the filtrate was concentrated under reduced pressure. The residue was purified by silica gel column chromatography eluted with DCM:MeOH (10:1) to afford tert-butyl 4-{4-[5-(2-chloropyrimidin-4-yl)-4-[2-fluoro-3-(propane-1-sulfonamido)phenyl]-1,3-thiazol-2-yl]phenyl}piperazine-1-carboxylate (1 g, 69.46%) as a yellow-green solid. LCMS: C₃₁H₃₄ClFN₆O₄S₂ requires: 672.2, found: m/z=673.2 [M+H]⁺.

### Step-4: Synthesis of tert-butyl 4-[4-(5-{2-[(tert-butoxycarbonyl)amino]pyrimidin-4-yl}-4-[2-fluoro-3-(propane-1-sulfonamido)phenyl]-1,3-thiazol-2-yl)phenyl]piperazine-1-carboxylate (5)

To a mixture of tert-butyl 4-{4-[5-(2-chloropyrimidin-4-yl)-4-[2-fluoro-3-(propane-1-sulfonamido)phenyl]-1,3-thiazol-2-yl]phenyl}piperazine-1-carboxylate (1 g, 1.48 mmol, 1 equiv) in dioxane (10 mL) was added tert-butyl carbamate (870 mg, 7.42 mmol, 5 equiv), Cs₂CO₃ (967 mg, 2.97 mmol, 2 equiv), and BrettPhos Pd G₃ (134 mg, 0.15 mmol, 0.1 equiv) at room temperature. The resulting mixture was stirred 2 h at 80° C. under nitrogen atmosphere. The resulting mixture was concentrated under reduced pressure. The residue was purified by reverse phase flash chromatography with H₂O:MeCN (1:7) to afford tert-butyl 4-[4-(5-{2-[(tert-butoxycarbonyl)amino]pyrimidin-4-yl}-4-[2-fluoro-3-(propane-1-sulfonamido)phenyl]-1,3-thiazol-2-yl)phenyl]piperazine-1-carboxylate (500 mg, 42.42%) as a yellow solid. LCMS: C₃₆H₄₄FN₇O₆S₂ requires: 753.3, found: m/z=754.2 [M+H]⁺.

### Step-5: Synthesis of N-{3-[5-(2-aminopyrimidin-4-yl)-2-[4-(piperazin-1-yl)phenyl]-1,3-thiazol-4-yl]-2-fluorophenyl}propane-1-sulfonamide Hydrochloride

A mixture of tert-butyl 4-[4-(5-{2-[(tert-butoxycarbonyl)amino]pyrimidin-4-yl}-4-[2-fluoro-3-(propane-1-sulfonamido)phenyl]-1,3-thiazol-2-yl)phenyl]piperazine-1-carboxylate (500 mg, 0.663 mmol, 1 equiv) in HCl/1,4-dioxane (10 mL, 4 M) was stirred overnight at room temperature. The resulting mixture was concentrated under reduced pressure to afford N-{3-[5-(2-aminopyrimidin-4-yl)-2-[4-(piperazin-1-yl)phenyl]-1,3-thiazol-4-yl]-2-fluorophenyl}propane-1-sulfonamide hydrochloride salt (383.5 mg, crude) as a red solid. LCMS: C₂₆H₂₈FN₇O₂S₂ requires: 553.2, found: m/z=554.2 [M+H]⁺. ¹H NMR (400 MHz, CD₃OD) δ 8.02-7.99 (m, 3H), 7.73-7.69 (m, 1H), 7.52-7.48 (m, 1H), 7.41-7.37 (m, 1H), 7.16-7.14 (m, 2H), 6.58-6.56 (m, 1H), 3.67-3.57 (m, 4H), 3.40-3.38 (m, 4H), 3.17-3.14 (m, 2H), 1.89-1.83 (m, 2H), 1.06-1.02 (m, 3H).

### Synthesis of 4-[4-(3-{[ethyl(methyl)sulfamoyl]amino}-2-fluorophenyl)-2-methyl-1,3-thiazol-5-yl]-N-[5-(piperazin-1-yl)pyridin-2-yl]pyrimidin-2-amine; Trifluoroacetic Acid Salt

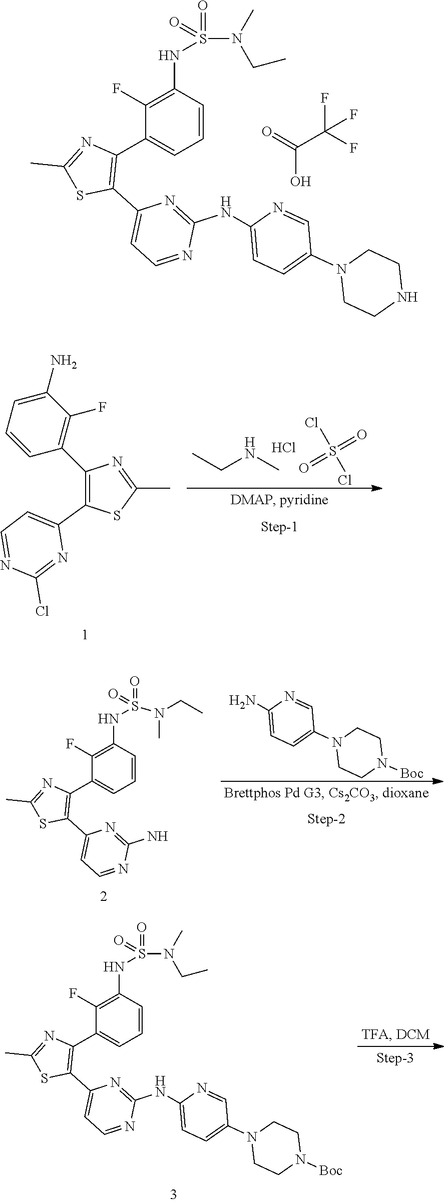

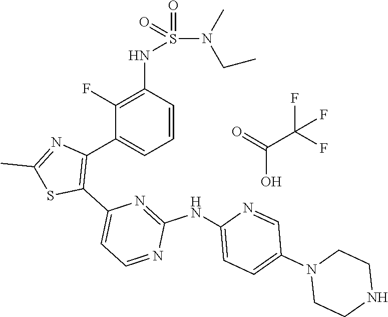

### Step-1: Synthesis of ({3-[5-(2-chloropyrimidin-4-yl)-2-methyl-1,3-thiazol-4-yl]-2-fluorophenyl}sulfamoyl)(ethyl)methylamine (2)

To a stirred mixture of 3-[5-(2-chloropyrimidin-4-yl)-2-methyl-1,3-thiazol-4-yl]-2-fluoroaniline (2.62 g, 8.162 mmol, 0.78 equiv) and methylethylamine hydrochloride (1 g, 10.464 mmol, 1 equiv) in pyridine (30 mL) was added DMAP (256 mg, 2.093 mmol, 0.2 equiv). The resulting mixture was stirred at −30° C. for 10 min under a nitrogen atmosphere. To the above mixture was added sulfonyl chloride (1.77 g, 13.08 mmol, 1.25 equiv) at −30° C. under the nitrogen atmosphere. The resulting mixture was stirred at −30° C. for 20 min under the nitrogen atmosphere and then was stirred at room temperature for one hour under the nitrogen atmosphere. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with PE:EtOAc (3:2) to afford ({3-[5-(2-chloropyrimidin-4-yl)-2-methyl-1,3-thiazol-4-yl]-2-fluorophenyl}sulfamoyl)(ethyl)methylamine (1.1 g, 79.82%) as a brown solid. LCMS: C₁₇H₁₇ClFN₅O₂S₂ requires: 441.1, found: m/z=442.2 [M+H]⁺.

### Step-2: Synthesis of tert-butyl 4-[6-({4-[4-(3-{[ethyl(methyl)sulfamoyl]amino}-2-fluorophenyl)-2-methyl-1,3-thiazol-5-yl]pyrimidin-2-yl}amino)pyridin-3-yl]piperazine-1-carboxylate (3)

To a stirred mixture of ({3-[5-(2-chloropyrimidin-4-yl)-2-methyl-1,3-thiazol-4-yl]-2-fluorophenyl}sulfamoyl)(ethyl)methylamine (1.1 g, 2.489 mmol, 1 equiv) and tert-butyl 4-(6-aminopyridin-3-yl)piperazine-1-carboxylate (901 mg, 3.236 mmol, 1.3 equiv) in dioxane (10 mL) was added Brettphos Pd G₃ (226 mg, 0.249 mmol, 0.1 equiv) and Cs₂CO₃ (1.62 g, 4.978 mmol, 2 equiv). The resulting mixture was stirred at 80° C. for one hour under a nitrogen atmosphere. The resulting mixture was concentrated under reduced pressure. The residue was purified by silica gel column chromatography eluted with PE:EtOAc (1:4) to afford tert-butyl 4-[6-({4-[4-(3-{[ethyl(methyl)sulfamoyl]amino}-2-fluorophenyl)-2-methyl-1,3-thiazol-5-yl]pyrimidin-2-yl}amino)pyridin-3-yl]piperazine-1-carboxylate (700 mg, 37.01%) as a yellow solid. LCMS: C₃₁H₃₈FN₉O₄S₂ requires: 683.3, found: m/z=684.4 [M+H]⁺.

### Step-3: Synthesis of 4-[4-(3-{[ethyl(methyl)sulfamoyl]amino}-2-fluorophenyl)-2-methyl-1,3-thiazol-5-yl]-N-[5-(piperazin-1-yl)pyridin-2-yl]pyrimidin-2-amine; Trifluoroacetic Acid

To a stirred mixture of tert-butyl 4-[6-({4-[4-(3-{[ethyl(methyl)sulfamoyl]amino}-2-fluorophenyl)-2-methyl-1,3-thiazol-5-yl]pyrimidin-2-yl}amino)pyridin-3-yl]piperazine-1-carboxylate (700 mg, 1.024 mmol, 1 equiv) in DCM (12 mL) was added TFA (4 mL) at 0° C. The resulting mixture was stirred at room temperature for one hour. The resulting mixture was concentrated under reduced pressure. The residue was purified by C18 reverse phase column chromatography with ACN:H₂O (1:3) to afford 4-[4-(3-{[ethyl(methyl)sulfamoyl]amino}-2-fluorophenyl)-2-methyl-1,3-thiazol-5-yl]-N-[5-(piperazin-1-yl)pyridin-2-yl]pyrimidin-2-amine; trifluoroacetic acid salt (520.4 mg, 71.04%) as an orange solid. LCMS: C₂₆H₃₀FN₉O₂S₂ requires: 583.2, found: m/z=584.2 [M+H]⁺.

¹H NMR (400 MHz, CD₃OD) δ 8.58-8.52 (m, 1H), 8.20-8.13 (m, 1H), 7.82-7.77 (m, 1H), 7.69-7.60 (m, 1H), 7.57-7.50 (m, 1H), 7.48-7.40 (m, 1H), 7.40-7.32 (m, 1H), 6.95-6.89 (m, 1H), 3.66-3.50 (m, 4H), 3.50-3.29 (m, 4H), 3.27-3.17 (m, 2H), 2.86-2.79 (m, 6H), 1.16-1.08 (m, 3H).

### Synthesis of 4-[4-(3-{[ethyl(methyl)sulfamoyl]amino}-2-fluorophenyl)-2-methyl-1,3-thiazol-5-yl]-N-[5-(piperidin-4-yl)pyridin-2-yl]pyrimidin-2-amine; Trifluoroacetic Acid Salt

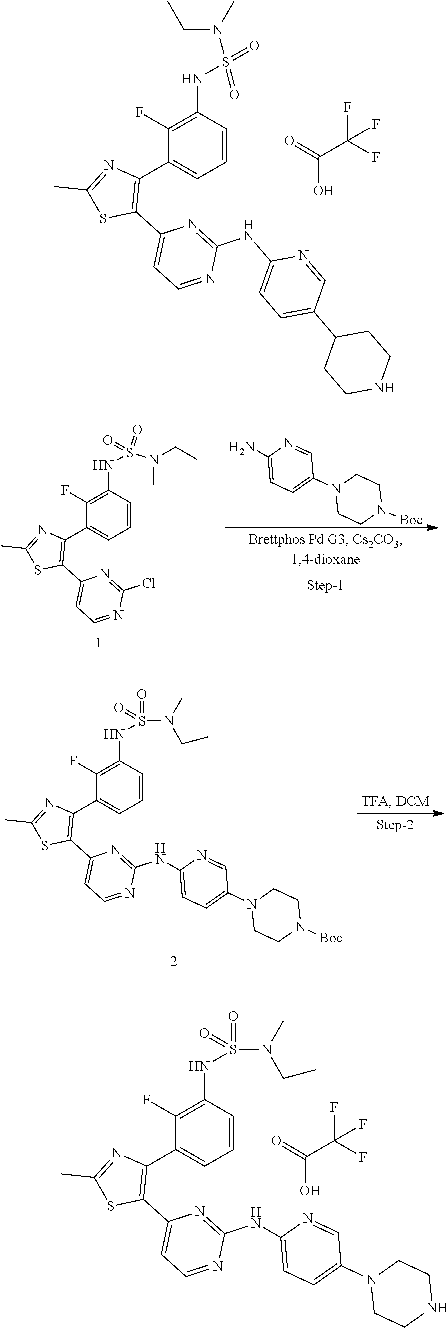

### Step-1: Synthesis of tert-butyl 4-[6-({4-[4-(3-{[ethyl(methyl)sulfamoyl]amino}-2-fluorophenyl)-2-methyl-1,3-thiazol-5-yl}pyrimidin-2-yl]amino)pyridin-3-yl]piperidine-1-carboxylate (2)

To a mixture of ({3-[5-(2-chloropyrimidin-4-yl)-2-methyl-1,3-thiazol-4-yl]-2-fluorophenyl}sulfamoyl)(ethyl)methylamine (300 mg, 0.679 mmol, 1 equiv) and tert-butyl 4-(6-aminopyridin-3-yl)piperidine-1-carboxylate (282 mg, 1.018 mmol, 1.5 equiv) in dioxane (10 mL) was added Brettphos Pd G₃ (62 mg, 0.068 mmol, 0.1 equiv) and Cs₂CO₃ (442 mg, 1.358 mmol, 2 equiv). The resulting mixture was stirred at 80° C. for 2 h under a nitrogen atmosphere. The resulting mixture was concentrated under reduced pressure. The residue was purified by silica gel column chromatography eluted with PE:EtOAc (1:4) to afford tert-butyl 4-[6-({4-[4-(3-{[ethyl(methyl)sulfamoyl]amino}-2-fluorophenyl)-2-methyl-1,3-thiazol-5-yl]pyrimidin-2-yl}amino)pyridin-3-yl]piperidine-1-carboxylate (350 mg, 67.95%) as a brown solid. LCMS: C₃₂H₃₉FN₈O₄S₂ requires: 682.3, found: m/z=683.2 [M+H]⁺.

### Step-2: Synthesis of 4-[4-(3-{[ethyl(methyl)sulfamoyl]amino}-2-fluorophenyl)-2-methyl-1,3-thiazol-5-yl]-N-[5-(piperidin-4-yl)pyridin-2-yl]pyrimidin-2-amine; Trifluoroacetic Acid

To a mixture of tert-butyl 4-[6-({4-[4-(3-{[ethyl(methyl)sulfamoyl]amino}-2-fluorophenyl)-2-methyl-1,3-thiazol-5-yl]pyrimidin-2-yl}amino)pyridin-3-yl]piperidine-1-carboxylate (340 mg, 0.498 mmol, 1 equiv) in DCM (6 mL) was added TFA (2 mL) at 0° C. under an air atmosphere. The resulting mixture was stirred at room temperature for one hour. The resulting mixture was concentrated under reduced pressure. This resulted in 4-[4-(3-{[ethyl(methyl)sulfamoyl]amino}-2-fluorophenyl)-2-methyl-1,3-thiazol-5-yl]-N-[5-(piperidin-4-yl)pyridin-2-yl]pyrimidin-2-amine; trifluoroacetic acid salt (319.4 mg, 91.05%) as a brown solid. LCMS: C₂₇H₃₁FN₈O₂S₂ requires: 582.2, found: m/z=583.2 [M+H]⁺. ¹H NMR (400 MHz, DMSO-d₆) δ 11.09-11.05 (m, 1H), 9.69-9.65 (m, 1H), 8.76-8.69 (m, 1H), 8.55-8.46 (m, 1H), 8.19-8.14 (m, 1H), 7.97-7.90 (m, 1H), 7.86-7.79 (m, 1H), 7.58-7.49 (m, 1H), 7.48-7.39 (m, 1H), 7.39-7.31 (m, 1H), 6.72-6.66 (m, 1H), 3.46-3.39 (m, 2H), 3.11-3.00 (m, 4H), 3.00-2.90 (m, 1H), 2.81-2.76 (m, 3H), 2.69-2.64 (m, 3H), 2.04-1.96 (m, 2H), 1.87-1.72 (m, 2H), 1.06-0.98 (m, 3H).

### Synthesis of 4-[4-(3-{[ethyl(methyl)sulfamoyl]amino}-2-fluorophenyl)-2-methyl-1,3-thiazol-5-yl]-N-[3-fluoro-4-(piperazin-1-yl)phenyl]pyrimidin-2-amine; Trifluoroacetic Acid Salt

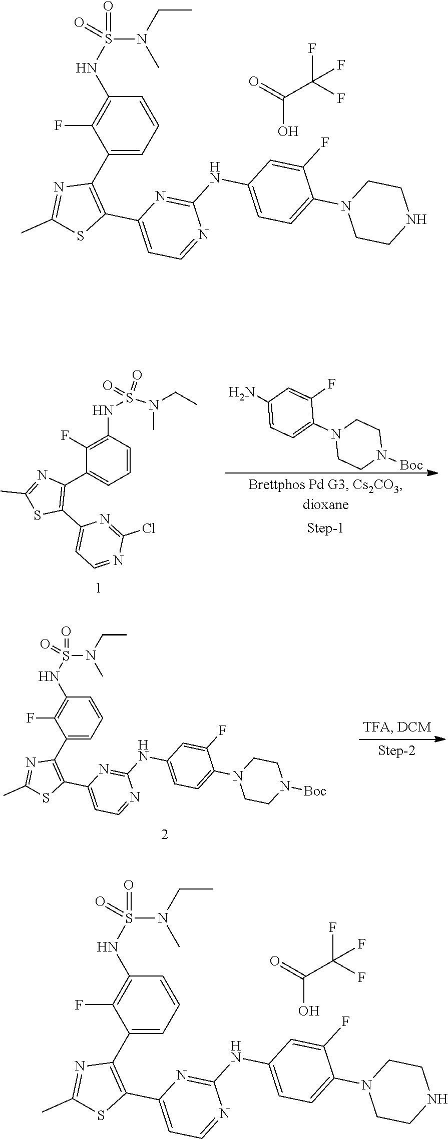

### Step-1: Synthesis of tert-butyl 4-[4-({4-[4-(3-{[ethyl(methyl)sulfamoyl]amino}-2-fluorophenyl)-2-methyl-1,3-thiazol-5-yl]pyrimidin-2-yl}amino)-2-fluorophenyl]piperazine-1-carboxylate (2)

To a stirred mixture of ({3-[5-(2-chloropyrimidin-4-yl)-2-methyl-1,3-thiazol-4-yl]-2-fluorophenyl}sulfamoyl)(ethyl)methylamine (450 mg, 1.018 mmol, 1 equiv) and tert-butyl 4-(4-amino-2-fluorophenyl)piperazine-1-carboxylate (451 mg, 1.527 mmol, 1.5 equiv) in dioxane (10 mL) was added Brettphos Pd G₃ (92 mg, 0.102 mmol, 0.1 equiv) and Cs₂CO₃ (664 mg, 2.036 mmol, 2 equiv). The resulting mixture was stirred at 80° C. for 2 h under a nitrogen atmosphere. The residue was purified by C18 reverse phase column chromatography with ACN:H₂O (7:3) to afford tert-butyl 4-[4-({4-[4-(3-{[ethyl(methyl)sulfamoyl]amino}-2-fluorophenyl)-2-methyl-1,3-thiazol-5-yl]pyrimidin-2-yl}amino)-2-fluorophenyl]piperazine-1-carboxylate (400 mg, 50.45%) as a yellow solid. LCMS: C₃₂H₃₈F₂N₈O₄S₂ requires: 700.2, found: m/z=701.2 [M+H]⁺.

### Step-2: Synthesis of 4-[4-(3-{[ethyl(methyl)sulfamoyl]amino}-2-fluorophenyl)-2-methyl-1,3-thiazol-5-yl]-N-[3-fluoro-4-(piperazin-1-yl)phenyl]pyrimidin-2-amine; Trifluoroacetic Acid

To a stirred mixture of tert-butyl 4-[4-({4-[4-(3-{[ethyl(methyl)sulfamoyl]amino}-2-fluorophenyl)-2-methyl-1,3-thiazol-5-yl]pyrimidin-2-yl}amino)-2-fluorophenyl]piperazine-1-carboxylate (400 mg, 0.571 mmol, 1 equiv) in DCM (6 mL) was added TFA (2 mL) at 0° C. The resulting mixture was stirred at room temperature for one hour. The resulting mixture was concentrated under reduced pressure. This resulted in 4-[4-(3-{[ethyl(methyl)sulfamoyl]amino}-2-fluorophenyl)-2-methyl-1,3-thiazol-5-yl]-N-[3-fluoro-4-(piperazin-1-yl)phenyl]pyrimidin-2-amine; trifluoroacetic acid salt (345.9 mg, crude) as an orange solid. LCMS: C₂₇H₃₀F₂N₈O₂S₂ requires: 600.2, found: m/z=601.1 [M+H]⁺.

¹H NMR (400 MHz, CD₃OD) δ 8.29-8.23 (m, 1H), 7.71-7.60 (m, 2H), 7.37-7.27 (m, 3H), 7.09-7.00 (m, 1H), 6.52-6.46 (m, 1H), 3.45-3.38 (m, 4H), 3.33-3.14 (m, 4H), 3.27-3.12 (m, 2H), 2.82-2.76 (m, 6H), 1.13-1.05 (m, 3H).

### Synthesis of N-{2-fluoro-3-[1-(4-formylphenyl)-3-(pyridin-4-yl)pyrazol-4-yl]phenyl}propane-1-sulfonamide

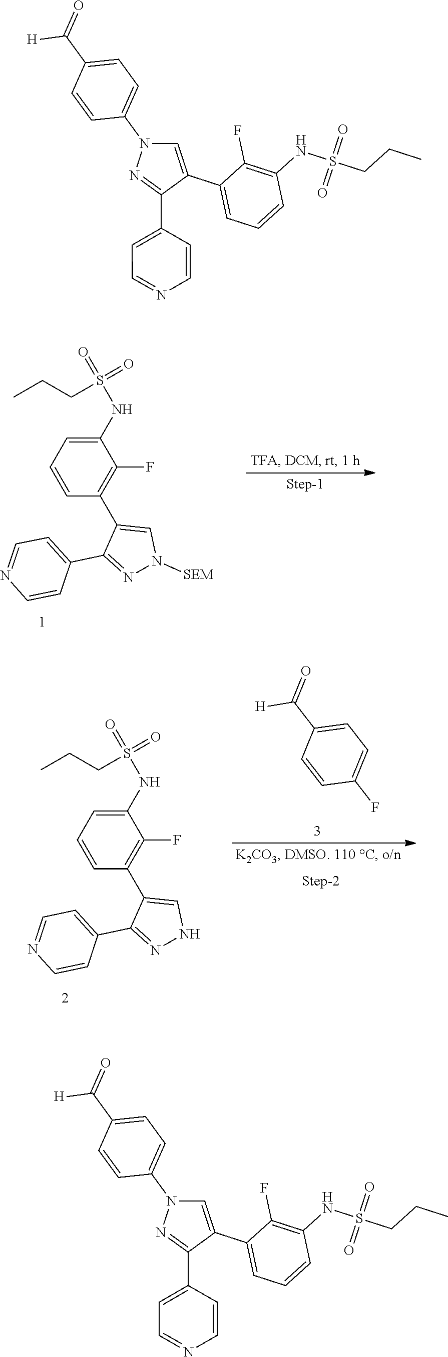 [SMILES: CCCS(=O)(=O)Nc1cccc(-c2cn(-c3ccc(C=O)cc3)nc2-c2ccncc2)c1F ; CCCS(=O)(=O)Nc1cccc(-c2cn(COCC[Si](C)(C)C)nc2-c2ccncc2)c1F ; CCCS(=O)(=O)Nc1cccc(-c2c[nH]nc2-c2ccncc2)c1F ; O=Cc1ccc(F)cc1 ; CCCS(=O)(=O)Nc1cccc(-c2cn(-c3ccc(C=O)cc3)nc2-c2ccncc2)c1F]

### Step-1: Synthesis of N-{2-fluoro-3-[3-(pyridin-4-yl)-1H-pyrazol-4-yl]phenyl}propane-1-sulfonamide (2)

To a mixture of N-{2-fluoro-3-[3-(pyridin-4-yl)-1-{[2-(trimethylsilyl)ethoxy]methyl}pyrazol-4-yl]phenyl}propane-1-sulfonamide (2.0 g, 4.076 mmol, 1 equiv) in DCM (15 mL) was added TFA (5 mL). The resulting mixture was stirred for one hour at room temperature. The resulting mixture was concentrated under reduced pressure. The residue was purified by silica gel column chromatography eluted with CH₂Cl₂:MeOH (8:1) to afford N-{2-fluoro-3-[3-(pyridin-4-yl)-1H-pyrazol-4-yl]phenyl}propane-1-sulfonamide (1.0 g, 68.07%) as a yellow solid. LCMS: C₁₇H₁₇FN₄O₂S requires: 360.1, found: m/z=361.1 [M+H]⁺.

### Step-2: Synthesis of N-{2-fluoro-3-[1-(4-formylphenyl)-3-(pyridin-4-yl)pyrazol-4-yl]phenyl}propane-1-sulfonamide

To a mixture of N-{2-fluoro-3-[3-(pyridin-4-yl)-1H-pyrazol-4-yl]phenyl}propane-1-sulfonamide (500 mg, 1.387 mmol, 1 equiv) and 4-fluorobenzaldehyde (516.55 mg, 4.161 mmol, 3.0 equiv) in DMSO (1 mL) was added K₂CO₃ (958.67 mg, 6.935 mmol, 5.0 equiv) in portions at room temperature. The resulting mixture was stirred at 110° C. overnight. The resulting mixture was extracted with EtOAc. The combined organic layers were washed with water and dried over anhydrous Na₂SO₄. After filtration, the filtrate was concentrated under reduced pressure. The residue was purified by reversed-phase flash chromatography with the following conditions: column, C18 silica gel; mobile phase, MeCN in water (10 mmol/L NH₄HCO₃), 10% to 50% gradient in 10 min; detector, UV 254 nm to afford N-{2-fluoro-3-[1-(4-formylphenyl)-3-(pyridin-4-yl)pyrazol-4-yl]phenyl}propane-1-sulfonamide (297.3 mg, 46.13%) as a light yellow solid. LCMS: C₂₄H₂₁FN₄O₃S requires: 464.1, found: m/z=465.1 [M+H]⁺. ¹H NMR (400 MHz, CD₃OD) δ 10.06 (s, 1H), 8.71 (s, 1H), 8.55 (d, J=1.8 Hz, 2H), 8.23-8.19 (s, 2H), 8.12 (d, J=8.6 Hz, 2H), 7.66-7.55 (m, 3H), 7.28-7.13 (m, 2H), 3.10-3.02 (m, 2H), 1.87-1.73 (m, 2H), 1.05-0.95 (m, 3H).

### Synthesis of N-(2-fluoro-3-{1-[4-(4-formylpiperidin-1-yl)phenyl]-3-(pyridin-4-yl)pyrazol-4-yl}phenyl)propane-1-sulfonamide

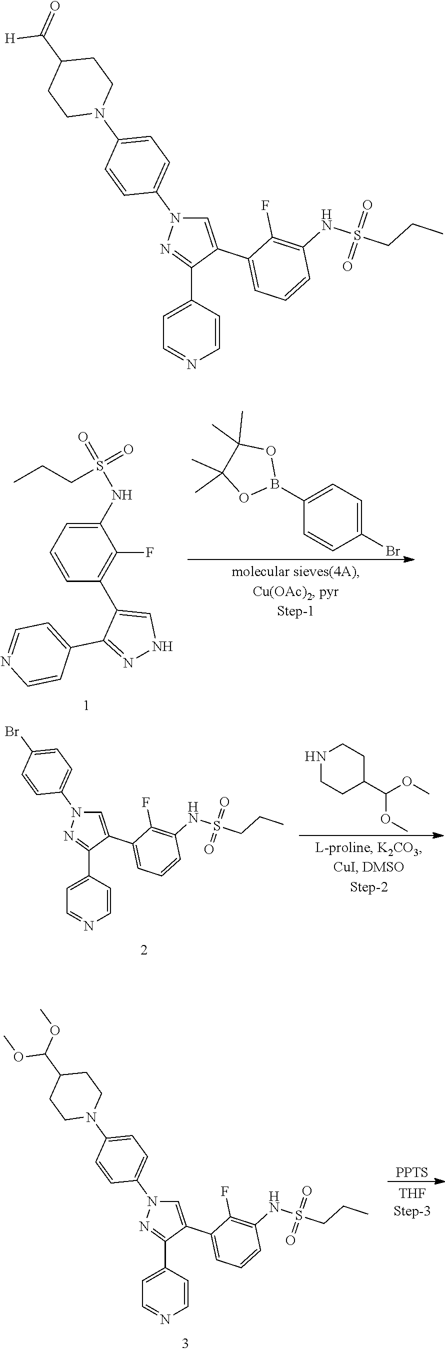

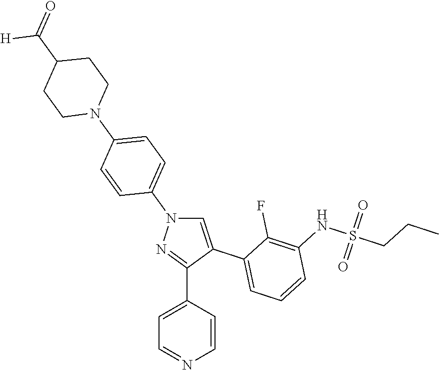 [SMILES: CCCS(=O)(=O)Nc1cccc(-c2cn(-c3ccc(N4CCC(C=O)CC4)cc3)nc2-c2ccncc2)c1F]

### Step-1: Synthesis of N-{3-[1-(4-bromophenyl)-3-(pyridin-4-yl)pyrazol-4-yl]-2-fluorophenyl}propane-1-sulfonamide (2)

To a stirred mixture of N-{2-fluoro-3-[3-(pyridin-4-yl)-1H-pyrazol-4-yl]phenyl}propane-1-sulfonamide (3 g, 8.324 mmol, 1 equiv) and 2-(4-bromophenyl)-4,4,5,5-tetramethyl-1,3,2-dioxaborolane (3.53 g, 12.486 mmol, 1.5 equiv) in pyridine (50 mL) was added Cu(OAc)₂ (3.02 g, 16.648 mmol, 2 equiv) and molecular sieves (4 A) (3 g). The resulting mixture was stirred at 100° C. overnight under an oxygen atmosphere. The resulting mixture was concentrated under reduced pressure. The residue was purified by silica gel column chromatography eluted with PE:EtOAc (1:1) to afford N-{3-[1-(4-bromophenyl)-3-(pyridin-4-yl)pyrazol-4-yl]-2-fluorophenyl}propane-1-sulfonamide (2.4 g, 39.16%) as a brown semi-solid. LCMS: C₂₃H₂₀BrFN₄O₂S requires: 514.1, found: m/z=515.0 [M+H]⁺.

### Step-2: Synthesis of N-[3-(1-{4-[4-(dimethoxymethyl)piperidin-1-yl]phenyl}-3-(pyridin-4-yl)pyrazol-4-yl)-2-fluorophenyl]propane-1-sulfonamide (3)

To a stirred mixture of N-{3-[1-(4-bromophenyl)-3-(pyridin-4-yl)pyrazol-4-yl]-2-fluorophenyl}propane-1-sulfonamide (2.4 g, 4.657 mmol, 1 equiv) and 4-(dimethoxymethyl)piperidine (1.11 g, 6.986 mmol, 1.5 equiv) in DMSO (20 mL) was added L-proline (214 mg, 1.863 mmol, 0.4 equiv), K₂CO₃ (1.93 g, 13.971 mmol, 3 equiv), and CuI (177 mg, 0.931 mmol, 0.2 equiv). The resulting mixture was stirred at 100° C. overnight under a nitrogen atmosphere. The resulting mixture was concentrated under reduced pressure. The residue was purified by silica gel column chromatography eluted with PE:EtOAc (4:1) to afford N-[3-(1-{4-[4-(dimethoxymethyl)piperidin-1-yl]phenyl}-3-(pyridin-4-yl)pyrazol-4-yl)-2-fluorophenyl]propane-1-sulfonamide (700 mg, 22.79%) as a brown solid. LCMS: C₃₁H₃₆FN₅O₄S requires: 593.3, found: m/z=594.2 [M+H]⁺.

### Step-3: Synthesis of N-(2-fluoro-3-{1-[4-(4-formylpiperidin-1-yl)phenyl]-3-(pyridin-4-yl)pyrazol-4-yl}phenyl)propane-1-sulfonamide

To a stirred mixture of N-[3-(1-{4-[4-(dimethoxymethyl)piperidin-1-yl]phenyl}-3-(pyridin-4-yl)pyrazol-4-yl)-2-fluorophenyl]propane-1-sulfonamide (500 mg, 0.842 mmol, 1 equiv) in THF (5 mL) was added PPTS (423 mg, 1.684 mmol, 2 equiv). The resulting mixture was stirred at 70° C. overnight under a nitrogen atmosphere. The residue was purified by C18 reverse phase column chromatography with ACN:H₂O (1:1) to afford N-(2-fluoro-3-{1-[4-(4-formylpiperidin-1-yl)phenyl]-3-(pyridin-4-yl)pyrazol-4-yl}phenyl)propane-1-sulfonamide (348.7 mg, 74.09%) as a light yellow solid. LCMS: C₂₉H₃₀FN₅O₃S requires: 547.2, found: m/z=548.2 [M+H]⁺. ¹H NMR (400 MHz, DMSO-d₆) δ 9.70-9.64 (m, 2H), 8.72-8.68 (m, 1H), 8.64-8.56 (m, 2H), 7.82-7.75 (m, 2H), 7.56-7.43 (m, 3H), 7.39-7.25 (m, 2H), 7.16-7.08 (m, 2H), 3.75-3.66 (m, 2H), 3.07-2.99 (m, 2H), 2.98-2.87 (m, 2H), 2.56-2.51 (m, 1H), 2.01-1.92 (m, 2H), 1.76-1.55 (m, 4H), 0.96-0.88 (m, 3H).

### Synthesis of 4-[4-(3-{[ethyl(methyl)sulfamoyl]amino}-2-fluorophenyl)-2-(piperidin-4-yl)-1,3-thiazol-5-yl]-N-isopropylpyrimidin-2-amine; Trifluoroacetic Acid Salt

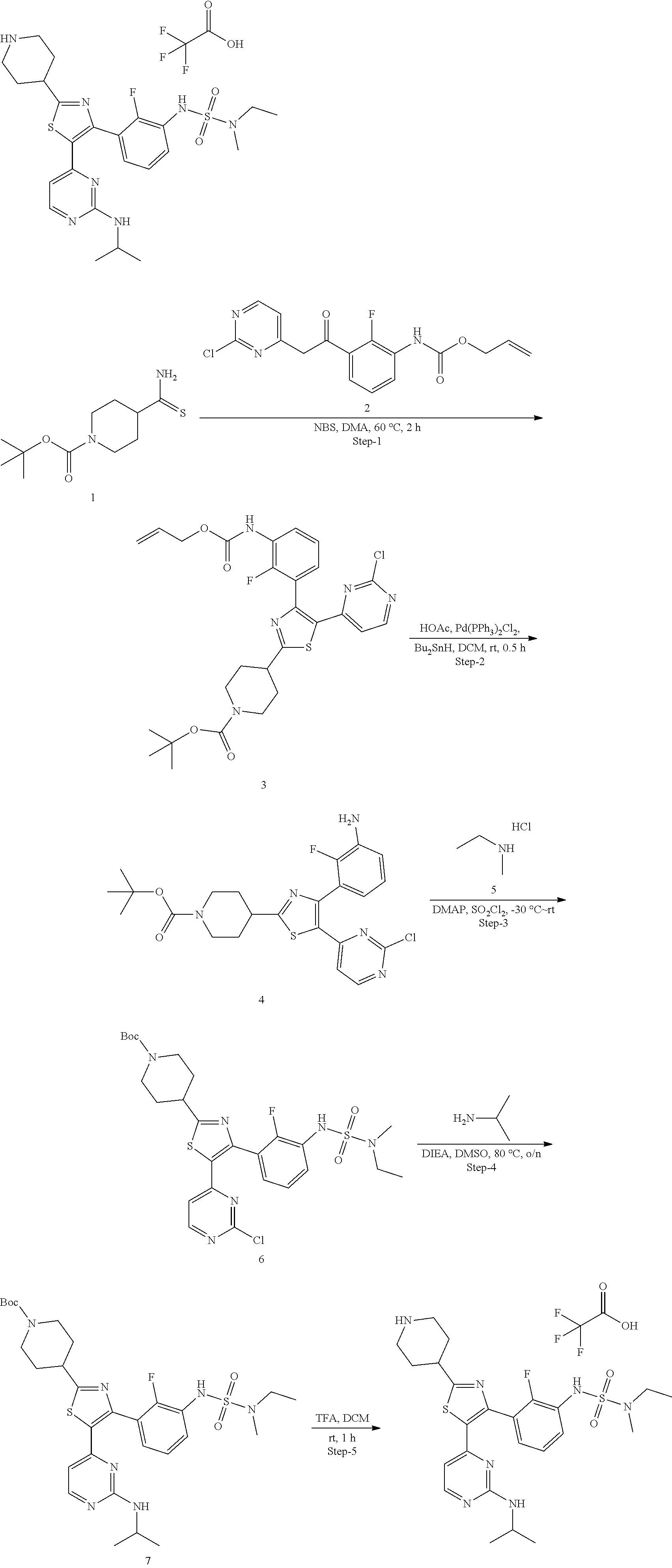

### Step-1: Synthesis of tert-butyl 4-[5-(2-chloropyrimidin-4-yl)-4-(2-fluoro-3-{[(prop-2-en-1-yloxy)carbonyl]amino}phenyl)-1,3-thiazol-2-yl]piperidine-1-carboxylate

A mixture of prop-2-en-1-yl N-{3-[2-(2-chloropyrimidin-4-yl)acetyl]-2-fluorophenyl}carbamate (4.29 g, 12.277 mmol, 1.0 equiv) and NBS (1.75 g, 9.822 mmol, 0.8 equiv) in DMA (15 mL) was stirred for 15 min at room temperature. To the above mixture was added tert-butyl 4-carbamothioylpiperidine-1-carboxylate (3.0 g, 12.277 mmol, 1 equiv) in portions at room temperature. The resulting mixture was stirred for an additional 2 h at room temperature. The reaction was quenched with water/ice at 0° C. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with PE:EtOAc (4:1) to afford tert-butyl 4-[5-(2-chloropyrimidin-4-yl)-4-(2-fluoro-3-{[(prop-2-en-1-yloxy)carbonyl]amino}phenyl)-1,3-thiazol-2-yl]piperidine-1-carboxylate (3.4 g, 48.23%) as a yellow solid. LCMS: C₂₇H₂₉ClFN₅O₄S requires: 573.2, found: m/z=574.2 [M+H]⁺.

### Step-2: Synthesis of tert-butyl 4-[4-(3-amino-2-fluorophenyl)-5-(2-chloropyrimidin-4-yl)-1,3-thiazol-2-yl]piperidine-1-carboxylate (4)

To a mixture of tert-butyl 4-[5-(2-chloropyrimidin-4-yl)-4-{3-[(ethoxycarbonyl)amino]-2-fluorophenyl}-1,3-thiazol-2-yl]piperidine-1-carboxylate (3.4 g, 6.049 mmol, 1 equiv) and Pd(PPh₃)₂Cl₂ (84.92 mg, 0.121 mmol, 0.02 equiv) in DCM (35 mL) was added HOAc (908.16 mg, 15.123 mmol, 2.5 equiv) dropwise at room temperature under a nitrogen atmosphere. To the above mixture was added n-Bu₃SnH (2817.14 mg, 9.678 mmol, 1.6 equiv) dropwise at 0° C. The resulting mixture was stirred for one hour at room temperature. The reaction was quenched with saturated aqueous sodium hyposulfite at 0° C. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with CH₂Cl₂:MeOH (10:1) to afford tert-butyl 4-[4-(3-amino-2-fluorophenyl)-5-(2-chloropyrimidin-4-yl)-1,3-thiazol-2-yl]piperidine-1-carboxylate (2.5 g, 84.34%) as a yellow solid. LCMS: C₂₃H₂₅ClFN₅O₂S requires: 489.1 found: m/z=490.1 [M+H]⁺.

### Step-3: Synthesis of tert-butyl 4-[5-(2-chloropyrimidin-4-yl)-4-(3-{[ethyl(methyl)sulfamoyl]amino}-2-fluorophenyl)-1,3-thiazol-2-yl]piperidine-1-carboxylate (6)

To a stirred mixture of tert-butyl 4-[4-(3-amino-2-fluorophenyl)-5-(2-chloropyrimidin-4-yl)-1,3-thiazol-2-yl]piperidine-1-carboxylate (1.0 g, 2.041 mmol, 1 equiv) and N-ethyl-N-methylsulfamoyl chloride (1.93 g, 12.246 mmol, 6.0 equiv, in situ) in DCM (10 mL) was added TEA (1.03 g, 10.205 mmol, 5.0 equiv) at room temperature. The resulting mixture was stirred for 3 h at 70° C. The reaction was quenched with water/ice at 0° C. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with CH₂Cl₂:EtOAc (2:1) to afford tert-butyl 4-[5-(2-chloropyrimidin-4-yl)-4-(3-{[ethyl(methyl)sulfamoyl]amino}-2-fluorophenyl)-1,3-thiazol-2-yl]piperidine-1-carboxylate (500 mg, 40.09%) as a yellow solid. LCMS: C₂₆H₃₂ClFN₆O₄S₂ requires: 610.2 found: m/z=611.2 [M+H]⁺.

### Step-4: Synthesis of tert-butyl 4-[4-(3-{[ethyl(methyl)sulfamoyl]amino}-2-fluorophenyl)-5-[2-(isopropylamino)pyrimidin-4-yl]-1,3-thiazol-2-yl]piperidine-1-carboxylate (7)

To a mixture of tert-butyl 4-[5-(2-chloropyrimidin-4-yl)-4-(3-{[ethyl(methyl)sulfamoyl]amino}-2-fluorophenyl)-1,3-thiazol-2-yl]piperidine-1-carboxylate (500 mg, 0.818 mmol, 1 equiv) and isopropylamine (145.08 mg, 2.454 mmol, 3.0 equiv) in DMSO (5 mL) was added DIEA (317.22 mg, 2.454 mmol, 3.0 equiv) dropwise at room temperature. The resulting mixture was stirred at 80° C. overnight. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with CH₂Cl₂:EtOAc (2:1) to afford tert-butyl 4-[4-(3-{[ethyl(methyl)sulfamoyl]amino}-2-fluorophenyl)-5-[2-(isopropylamino)pyrimidin-4-yl]-1,3-thiazol-2-yl]piperidine-1-carboxylate (350 mg, 67.50%) as a yellow solid. LCMS: C₂₉H₄₀FN₇O₄S₂ requires: 633.3 found: m/z=634.3 [M+H]⁺.

### Step-5: Synthesis of 4-[4-(3-{[ethyl(methyl)sulfamoyl]amino}-2-fluorophenyl)-2-(piperidin-4-yl)-1,3-thiazol-5-yl]-N-isopropylpyrimidin-2-amine; Trifluoroacetic Acid

To a mixture of tert-butyl 4-[4-(3-{[ethyl(methyl)sulfamoyl]amino}-2-fluorophenyl)-5-[2-(isopropylamino)pyrimidin-4-yl]-1,3-thiazol-2-yl]piperidine-1-carboxylate (350 mg, 0.552 mmol, 1 equiv) in DCM (2 mL) was added TFA (2 mL). The resulting mixture was stirred at room temperature for one hour. The resulting mixture was concentrated under reduced pressure. The residue was purified by reversed-phase flash chromatography with the following conditions: column, C18 silica gel; mobile phase, MeCN in water (0.1% TFA), 10% to 50% gradient in 10 min; detector, UV 254 nm to afford 4-[4-(3-{[ethyl(methyl)sulfamoyl]amino}-2-fluorophenyl)-2-(piperidin-4-yl)-1,3-thiazol-5-yl]-N-isopropylpyrimidin-2-amine; trifluoroacetic acid salt (339.6 mg, 94.94%) as a yellow solid. LCMS: C₂₄H₃₂FN₇O₂S₂ requires: 533.2 found: m/z=534.2 [M+H]⁺. ¹H NMR (400 MHz, CD₃OD) δ 8.07 (d, J=6.2 Hz, 1H), 7.72-7.62 (m, 1H), 7.38-7.27 (m, 2H), 6.48 (s, 1H), 4.05-3.95 (m, 1H), 3.58-3.50 (m, 3H), 3.26-3.15 (m, 4H), 2.85-2.77 (m, 3H), 2.41-2.31 (m, 2H), 2.19-2.04 (m, 2H), 1.24-1.18 (m, 6H), 1.21-1.05 (m, 3H).

### Synthesis of N-(2-fluoro-3-{1-[6-(piperazin-1-yl)pyridin-3-yl]-3-(pyridin-4-yl)pyrazol-4-yl}phenyl)propane-1-sulfonamide; Trifluoroacetic Acid Salt

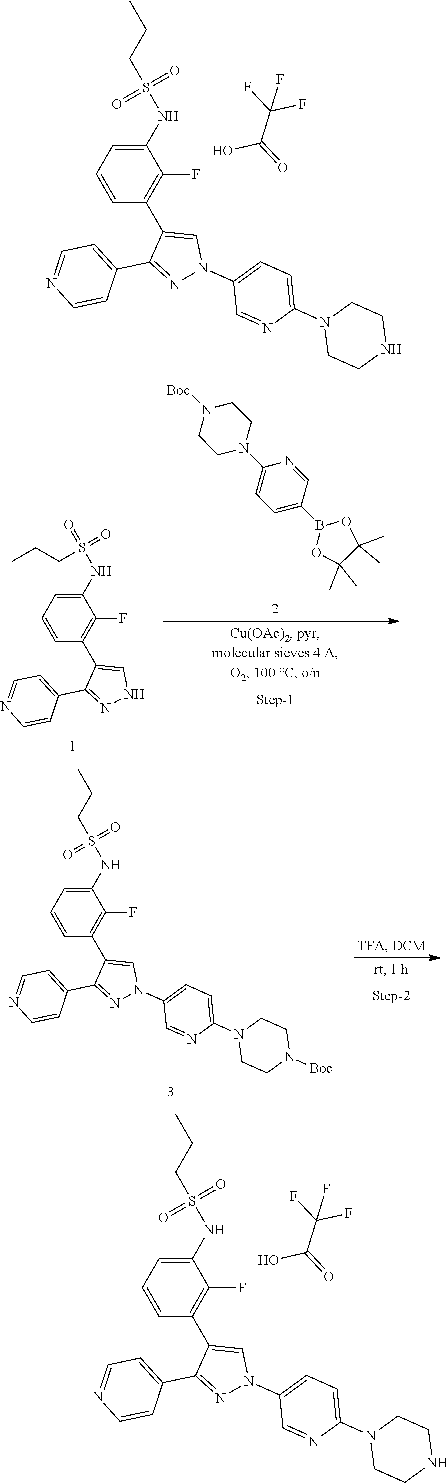

### Step-1: Synthesis of tert-butyl 4-(5-{4-[2-fluoro-3-(propane-1-sulfonamido)phenyl]-3-(pyridin-4-yl)pyrazol-1-yl}pyridin-2-yl)piperazine-1-carboxylate

To a mixture of N-{2-fluoro-3-[3-(pyridin-4-yl)-1H-pyrazol-4-yl]phenyl}propane-1-sulfonamide (600 mg, 1.665 mmol, 1 equiv) and tert-butyl 4-[5-(4,4,5,5-tetramethyl-1,3,2-dioxaborolan-2-yl)pyridin-2-yl]piperazine-1-carboxylate (972.14 mg, 2.498 mmol, 1.5 equiv) in pyridine (4 mL) was added Cu(OAc)₂ (604.76 mg, 3.330 mmol, 2.0 equiv) and molecular sieves 4 A (600 mg) at room temperature. The resulting mixture was stirred at 100° C. overnight under an oxygen atmosphere. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with CH₂Cl₂:MeOH (10:1) to afford tert-butyl 4-(5-{4-[2-fluoro-3-(propane-1-sulfonamido)phenyl]-3-(pyridin-4-yl)pyrazol-1-yl}pyridin-2-yl)piperazine-1-carboxylate (440 mg, 42.51%) as a white solid. LCMS: C₃₁H₃₆FN₇O₄S requires: 621.3, found: m/z=622.3 [M+H]⁺.

### Step-2: Synthesis of N-(2-fluoro-3-{1-[6-(piperazin-1-yl)pyridin-3-yl]-3-(pyridin-4-yl)pyrazol-4-yl}phenyl)propane-1-sulfonamide; Trifluoroacetic Acid

To a mixture of tert-butyl 4-(5-{4-[2-fluoro-3-(propane-1-sulfonamido)phenyl]-3-(pyridin-4-yl)pyrazol-1-yl}pyridin-2-yl)piperazine-1-carboxylate (440 mg, 0.708 mmol, 1 equiv) in DCM (3 mL) was added TFA (3 mL). The resulting mixture was stirred for one hour at room temperature. The resulting mixture was concentrated under reduced pressure. The residue was purified by reverse-phase flash chromatography with the following conditions: column, C18 silica gel; mobile phase, MeCN in Water (0.1% TFA), 10% to 50% gradient in 10 min; detector, UV 254 nm to afford N-(2-fluoro-3-{1-[6-(piperazin-1-yl)pyridin-3-yl]-3-(pyridin-4-yl)pyrazol-4-yl}phenyl)propane-1-sulfonamide; trifluoroacetic acid salt (314.4 mg, 68.03%) as a yellow solid. LCMS: C₂₆H₂₈FN₇O₂S requires: 521.2, found: m/z=522.1 [M+H]⁺. ¹H NMR (400 MHz, CD₃OD) δ 8.78 (s, 1H), 8.76-8.69 (m, 2H), 8.58 (s, 1H), 8.24-8.18 (m, 1H), 8.16 (d, J=6.8 Hz, 2H), 7.62-7.56 (m, 1H), 7.43-7.28 (m, 2H), 7.12 (d, J=9.2 Hz, 1H), 4.08-3.80 (m, 4H), 3.48-3.32 (m, 4H), 3.21-3.06 (m, 2H), 1.99-1.70 (m, 2H), 1.10-0.92 (m, 3H).

### Synthesis of N-{3-[5-(2-aminopyrimidin-4-yl)-2-[2-(piperidin-4-yl)ethynyl]-1,3-thiazol-4-yl]-2-fluorophenyl}propane-1-sulfonamide; Trifluoroacetic Acid Salt

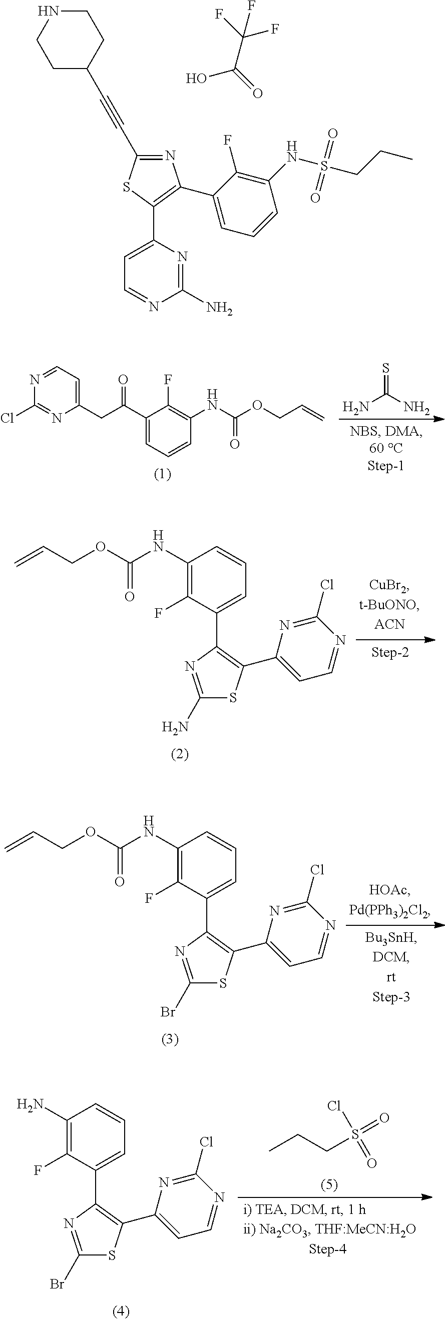

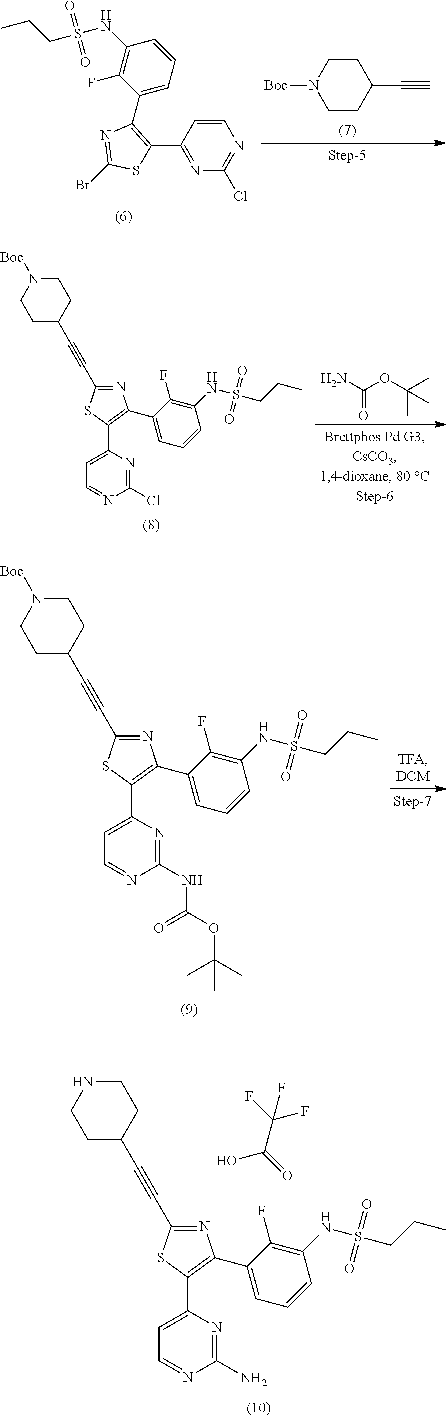

### Step-2: Synthesis of prop-2-en-1-yl N-{3-[2-bromo-5-(2-chloropyrimidin-4-yl)-1,3-thiazol-4-yl]-2-fluorophenyl}carbamate (3)

To a mixture of prop-2-en-1-yl N-{3-[2-amino-5-(2-chloropyrimidin-4-yl)-1,3-thiazol-4-yl]-2-fluorophenyl}carbamate (3 g, 7.392 mmol, 1 equiv) in MeCN (30 mL) was added CuBr₂ (825.55 mg, 3.696 mmol, 0.5 equiv) and t-BuONO (762.30 mg, 7.392 mmol, 1 equiv) at 0° C. The resulting mixture was stirred for 3 h at 0° C. The resulting mixture was concentrated under vacuum. The residue was purified by silica gel column chromatography eluted with CH₂Cl₂:EtOAc (1:1) to afford prop-2-en-1-yl N-{3-[2-bromo-5-(2-chloropyrimidin-4-yl)-1,3-thiazol-4-yl]-2-fluorophenyl}carbamate (1.5 g, 43.20%) as an off-white solid. LCMS: C₁₇H₁₁BrClFN₄O₂S requires: 468.0, found: m/z=469.0 [M+H]⁺.

### Step-3: Synthesis of 3-[2-bromo-5-(2-chloropyrimidin-4-yl)-1,3-thiazol-4-yl]-2-fluoroaniline (4)

To a mixture of prop-2-en-1-yl N-{3-[2-bromo-5-(2-chloropyrimidin-4-yl)-1,3-thiazol-4-yl]-2-fluorophenyl}carbamate (1.2 g, 2.555 mmol, 1 equiv) and Pd(PPh₃)₂Cl₂ (0.04 g, 0.051 mmol, 0.02 equiv) in DCM (10 mL) was added HOAc (0.40 g, 6.643 mmol, 2.6 equiv) and n-Bu₃SnH (1.19 g, 4.088 mmol, 1.6 equiv) at 0° C. under a nitrogen atmosphere. The resulting mixture was stirred for 30 min at room temperature under nitrogen atmosphere. The resulting mixture was extracted with EtOAc. The combined organic layers were washed with saturated aqueous NaHCO₃ and dried over anhydrous Na₂SO₄. After filtration, the filtrate was concentrated under reduced pressure. The residue was purified by silica gel column chromatography eluted with CH₂Cl₂:MeOH (10:1) to afford 3-[2-bromo-5-(2-chloropyrimidin-4-yl)-1,3-thiazol-4-yl]-2-fluoroaniline (603 mg, 61.20%) as a yellow solid. LCMS: C₁₃H₇BrClFN₄S requires: 383.9, found: m/z=385.0 [M+H]⁺.

### Step-4: Synthesis of N-{3-[2-bromo-5-(2-chloropyrimidin-4-yl)-1,3-thiazol-4-yl]-2-fluorophenyl}propane-1-sulfonamide (6)

To a mixture of 3-[2-bromo-5-(2-chloropyrimidin-4-yl)-1,3-thiazol-4-yl]-2-fluoroaniline (603 mg, 1.564 mmol, 1 equiv) and TEA (791.14 mg, 7.820 mmol, 5 equiv) in DCM (650 mL) was added propane-1-sulfonyl chloride (267.57 mg, 1.877 mmol, 1.2 equiv) at 0° C. The resulting mixture was stirred for one hour at room temperature. The resulting mixture was concentrated under vacuum. To the above mixture was added Na₂CO₃ (497.18 mg, 4.692 mmol, 3 equiv) in THF (10 mL), MeCN (10 mL), and H₂O (10 mL) at room temperature. The resulting mixture was stirred overnight at 50° C. The resulting mixture was extracted with CH₂Cl₂. The combined organic layers were washed with water and dried over anhydrous Na₂SO₄. After filtration, the filtrate was concentrated under reduced pressure. The residue was purified by silica gel column chromatography eluted with CH₂Cl₂:MeOH (10:1) to afford N-{3-[2-bromo-5-(2-chloropyrimidin-4-yl)-1,3-thiazol-4-yl]-2-fluorophenyl}propane-1-sulfonamide (701 mg, 91.16%) as a yellow solid. LCMS: C₁₆H₁₃BrClFN₄O₂S₂ requires: 489.9, found: m/z=491.0 [M+H]⁺.

### Step-5: Synthesis of tert-butyl 4-{2-[5-(2-chloropyrimidin-4-yl)-4-[2-fluoro-3-(propane-1-sulfonamido)phenyl]-1,3-thiazol-2-yl]ethynyl}piperidine-1-carboxylate (8)

To a mixture of N-{3-[2-bromo-5-(2-chloropyrimidin-4-yl)-1,3-thiazol-4-yl]-2-fluorophenyl}propane-1-sulfonamide (710 mg, 1.444 mmol, 1 equiv) and tert-butyl 4-ethynylpiperidine-1-carboxylate (453.24 mg, 2.166 mmol, 1.5 equiv) in THF (300 mL) was added TEA (100 mL), Pd(PPh₃)₄(250.25 mg, 0.217 mmol, 0.15 equiv), and CuI (82.49 mg, 0.433 mmol, 0.3 equiv) at room temperature under a nitrogen atmosphere. The resulting mixture was stirred at room temperature overnight under the nitrogen atmosphere. The resulting mixture was concentrated under reduced pressure. The residue was purified by silica gel column chromatography eluted with CH₂Cl₂:MeOH (10:1) to afford tert-butyl 4-{2-[5-(2-chloropyrimidin-4-yl)-4-[2-fluoro-3-(propane-1-sulfonamido)phenyl]-1,3-thiazol-2-yl]ethynyl}piperidine-1-carboxylate (587 mg, 65.56%) as a yellow solid. LCMS: C₂₈H₃₁ClFN₅O₄S₂ requires: 619.2, found: m/z=620.1 [M+H]⁺.

### Step-6: Synthesis of tert-butyl 4-[2-(5-{2-[(tert-butoxycarbonyl)amino]pyrimidin-4-yl}-4-[2-fluoro-3-(propane-1-sulfonamido)phenyl]-1,3-thiazol-2-yl)ethynyl]piperidine-1-carboxylate (9)

To a mixture of tert-butyl 4-{2-[5-(2-chloropyrimidin-4-yl)-4-[2-fluoro-3-(propane-1-sulfonamido)phenyl]-1,3-thiazol-2-yl]ethynyl}piperidine-1-carboxylate (587 mg, 0.947 mmol, 1 equiv) and tert-butyl carbamate (554.42 mg, 4.735 mmol, 5 equiv) in dioxane (15 mL) was added BrettPhos Pd G3 (85.80 mg, 0.095 mmol, 0.1 equiv) and Cs₂CO₃ (616.79 mg, 1.894 mmol, 2 equiv) at room temperature under a nitrogen atmosphere. The resulting mixture was stirred for 2 h at 80° C. under the nitrogen atmosphere. The resulting mixture was concentrated under reduced pressure. The residue was purified by silica gel column chromatography eluted with CH₂Cl₂:MeOH (10:1). The residue product was purified by reverse phase flash chromatography with MeCN:H₂O (65:35) to afford tert-butyl 4-[2-(5-{2-[(tert-butoxycarbonyl)amino]pyrimidin-4-yl}-4-[2-fluoro-3-(propane-1-sulfonamido)phenyl]-1,3-thiazol-2-yl)ethynyl]piperidine-1-carboxylate (270 mg, 40.70%) as a yellow solid. LCMS: C₃₃H₄₁FN₆O₆S₂ requires: 700.3, found: m/z=701.3 [M+H]⁺.

### Step-7: Synthesis of N-{3-[5-(2-aminopyrimidin-4-yl)-2-[2-(piperidin-4-yl)ethynyl]-1,3-thiazol-4-yl]-2-fluorophenyl}propane-1-sulfonamide; Trifluoroacetic Acid (10)

To a mixture of tert-butyl 4-[2-(5-{2-[(tert-butoxycarbonyl)amino]pyrimidin-4-yl}-4-[2-fluoro-3-(propane-1-sulfonamido)phenyl]-1,3-thiazol-2-yl)ethynyl]piperidine-1-carboxylate (270 mg, 0.385 mmol, 1 equiv) in DCM (6 mL) was added TFA (2 mL) at room temperature. The resulting mixture was stirred for one hour at room temperature. The resulting mixture was concentrated under vacuum. This resulted in N-{3-[5-(2-aminopyrimidin-4-yl)-2-[2-(piperidin-4-yl)ethynyl]-1,3-thiazol-4-yl]-2-fluorophenyl}propane-1-sulfonamide; trifluoroacetic acid (297.6 mg, crude) as a yellow solid. LCMS: C₂₃H₂₅FN₆O₂S₂ requires: 500.2, found: m/z=501.0 [M+H]⁺. ¹H NMR (400 MHz, CD₃OD) δ 8.11 (d, J=5.6 Hz, 1H), 7.71-7.68 (m, 1H), 7.40-7.33 (m, 2H), 6.40-6.38 (m, 1H), 3.47-3.41 (m, 2H), 3.24-3.17 (m, 3H), 3.14-3.10 (m, 2H), 2.27-2.22 (m, 2H), 2.10-1.96 (m, 2H), 1.88-1.82 (m, 2H), 1.05-1.02 (m, 3H).

### Synthesis of N-[3-(1-{3-azaspiro[5.5]undecan-9-yl}-3-(pyridin-4-yl)pyrazol-4-yl)-2-fluorophenyl]propane-1-sulfonamide; Trifluoroacetic Acid Salt

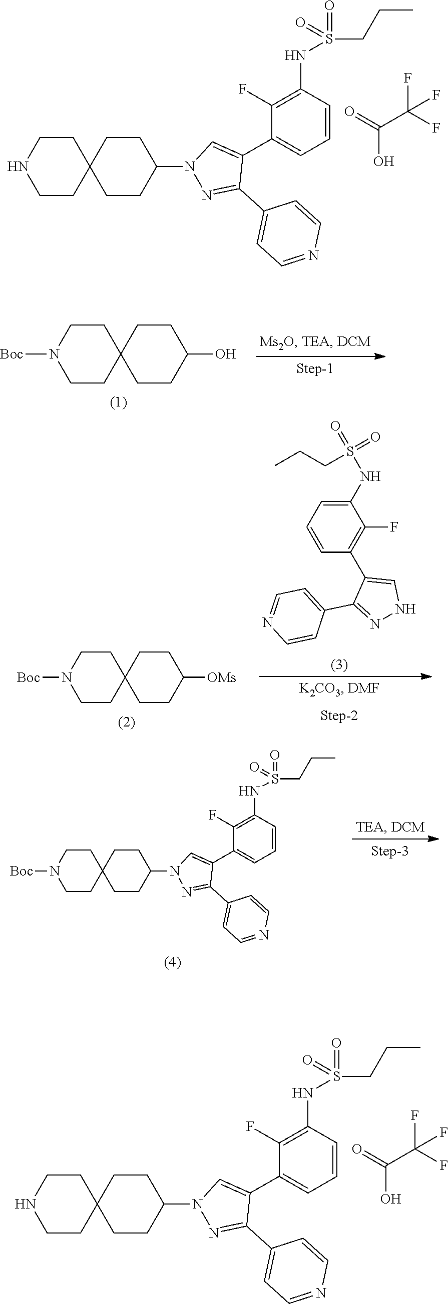

### Step-1: Synthesis of tert-butyl 9-(methanesulfonyloxy)-3-azaspiro[5.5]undecane-3-carboxylate (2)

To a mixture of tert-butyl 9-hydroxy-3-azaspiro[5.5]undecane-3-carboxylate (1.0 g, 3.712 mmol, 1 equiv) and TEA (563.47 mg, 5.568 mmol, 1.5 equiv) in DCM (5 mL) was added methanesulfonic anhydride (711.28 mg, 4.083 mmol, 1.1 equiv) dropwise at 0° C. The resulting mixture was stirred for 3 h at room temperature. The reaction was quenched by the addition of H₂O at 0° C. The resulting mixture was extracted with CH₂Cl₂. The combined organic layers were washed with water and dried over anhydrous Na₂SO₄. After filtration, the filtrate was concentrated under reduced pressure to afford tert-butyl 9-(methanesulfonyloxy)-3-azaspiro[5.5]undecane-3-carboxylate (1.15 g, crude) as a white solid.

### Step-2: Synthesis of tert-butyl 9-{4-[2-fluoro-3-(propane-1-sulfonamido)phenyl]-3-(pyridin-4-yl)pyrazol-1-yl}-3-azaspiro[5.5]undecane-3-carboxylate (4)

To a mixture of tert-butyl 9-(methanesulfonyloxy)-3-azaspiro[5.5]undecane-3-carboxylate (2603.06 mg, 7.494 mmol, 6.0 equiv) and N-{2-fluoro-3-[3-(pyridin-4-yl)-1H-pyrazol-4-yl]phenyl}propane-1-sulfonamide (450 mg, 1.249 mmol, 1.00 equiv) in DMF (5 mL) was added K₂CO₃ (345.12 mg, 2.498 mmol, 2.0 equiv) at room temperature. The resulting mixture was stirred at 80° C. overnight. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with CH₂Cl₂:MeOH (10:1) to afford tert-butyl 9-{4-[2-fluoro-3-(propane-1-sulfonamido)phenyl]-3-(pyridin-4-yl)pyrazol-1-yl}-3-azaspiro[5.5]undecane-3-carboxylate (221 mg, 28.93%) as a yellow solid. LCMS: C₃₂H₄₂FN₅O₄S requires: 611.3, found: m/z=612.0 [M+H]⁺.

### Step-3: Synthesis of N-[3-(1-{3-azaspiro[5.5]undecan-9-yl}-3-(pyridin-4-yl)pyrazol-4-yl)-2-fluorophenyl]propane-1-sulfonamide; Trifluoroacetic Acid

To a stirred mixture of tert-butyl 9-{4-[2-fluoro-3-(propane-1-sulfonamido)phenyl]-3-(pyridin-4-yl)pyrazol-1-yl}-3-azaspiro[5.5]undecane-3-carboxylate (200 mg, 0.327 mmol, 1 equiv) in DCM (0.5 mL) was added TFA (0.5 mL) at room temperature. The resulting mixture was stirred for one hour at room temperature. The resulting mixture was concentrated under reduced pressure. The residue was purified by silica gel column chromatography eluted with CH₃CN:H₂O (10:1) to afford N-[3-(1-{3-azaspiro[5.5]undecan-9-yl}-3-(pyridin-4-yl)pyrazol-4-yl)-2-fluorophenyl]propane-1-sulfonamide; trifluoroacetic acid salt (165.3 mg, 78.96%) as an off-white solid. LCMS: C₂₇H₃₄FN₅O₂S requires: 511.2, found: m/z=512.2 [M+H]⁺. ¹H NMR (400 MHz, CD₃OD) δ 8.68-8.60 (m, 2H), 8.08-7.92 (m, 3H), 7.62-7.58 (m, 1H), 7.49-7.45 (m, 1H), 7.41-7.35 (m, 1H), 3.97-3.91 (m, 1H), 3.25-3.11 (m, 4H), 3.08-3.02 (m, 2H), 2.03-1.93 (m, 1H), 1.88-1.72 (m, 5H), 1.58-1.52 (m, 4H), 1.43-1.30 (m, 3H), 1.27-1.23 (m, 1H), 1.08-1.02 (m, 3H).

### Synthesis of 4-[4-(3-{[ethyl(methyl)sulfamoyl]amino}-2-fluorophenyl)-1,3-thiazol-5-yl]-N-[5-(piperazin-1-yl)pyridin-2-yl]pyrimidin-2-amine; trifluoroacetic acid Salt

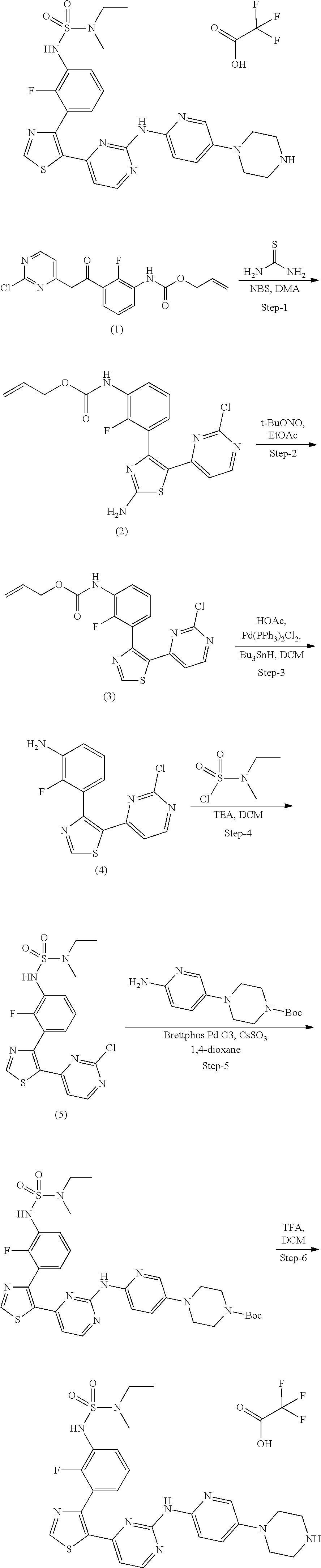

### Step-1: Synthesis of prop-2-en-1-yl N-{3-[2-amino-5-(2-chloropyrimidin-4-yl)-1,3-thiazol-4-yl]-2-fluorophenyl}carbamate (2)

To a mixture of prop-2-en-1-yl N-{3-[2-(2-chloropyrimidin-4-yl)acetyl]-2-fluorophenyl}carbamate (5.0 g, 14.296 mmol, 1 equiv) and NBS (2.54 g, 14.296 mmol, 1.0 equiv) in DMA (40 mL) was stirred for 15 min at 0° C. To the above mixture was added thiourea (1.31 g, 17.155 mmol, 1.2 equiv) in portions at 0° C. The resulting mixture was stirred for an additional 2 h at room temperature. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with CH₂Cl₂:MeOH (10:1) to afford prop-2-en-1-yl N-{3-[2-amino-5-(2-chloropyrimidin-4-yl)-1,3-thiazol-4-yl]-2-fluorophenyl}carbamate (3.8 g, 65.50%) as a yellow solid. LCMS: C₁₇H₁₃ClFN₅O₂S requires: 405.1, found: m/z=406.0 [M+H]⁺.

### Step-2: Synthesis of prop-2-en-1-yl N-{3-[5-(2-chloropyrimidin-4-yl)-1,3-thiazol-4-yl]-2-fluorophenyl}carbamate (3)

To a mixture of prop-2-en-1-yl N-{3-[2-amino-5-(2-chloropyrimidin-4-yl)-1,3-thiazol-4-yl]-2-fluorophenyl}carbamate (3.0 g, 7.392 mmol, 1 equiv) in EtOAc (30.0 mL) was added tert-butyl nitrite (1.14 g, 11.088 mmol, 1.5 equiv) in portions at room temperature. The resulting mixture was stirred for 4 h at 50° C. The resulting mixture was concentrated under reduced pressure. The residue was purified by silica gel column chromatography eluted with CH₂Cl₂:EtOAc (3:1) to afford prop-2-en-1-yl N-{3-[5-(2-chloropyrimidin-4-yl)-1,3-thiazol-4-yl]-2-fluorophenyl}carbamate (1.5 g, 51.92%) as a yellow solid. LCMS: C₁₇H₁₂ClFN₄O₂S requires: 390.0, found: m/z=391.0 [M+H]⁺.

### Step-3: Synthesis of 3-[5-(2-chloropyrimidin-4-yl)-1,3-thiazol-4-yl]-2-fluoroaniline (4)

To a mixture of prop-2-en-1-yl N-{3-[5-(2-chloropyrimidin-4-yl)-1,3-thiazol-4-yl]-2-fluorophenyl}carbamate (1.5 g, 3.838 mmol, 1 equiv) and HOAc (0.58 g, 9.595 mmol, 2.5 equiv) in DCM (15 mL) was added n-Bu₃SnH (1.79 g, 6.141 mmol, 1.6 equiv) dropwise at 0° C. under a nitrogen atmosphere. The resulting mixture was stirred for one hour at room temperature under the nitrogen atmosphere. The reaction was quenched by the addition of sat. sodium hyposulfite at 0° C. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with CH₂Cl₂:MeOH (10:1) to afford 3-[5-(2-chloropyrimidin-4-yl)-1,3-thiazol-4-yl]-2-fluoroaniline (800 mg, 78.14%) as a yellow oil. LCMS: C₁₃H₈ClFN₄S requires: 306.0, found: m/z=307.1 [M+H]⁺.

### Step-4: Synthesis of ({3-[5-(2-chloropyrimidin-4-yl)-1,3-thiazol-4-yl]-2-fluorophenyl}sulfamoyl) (ethyl)methylamine (5)

To a mixture of 3-[5-(2-chloropyrimidin-4-yl)-1,3-thiazol-4-yl]-2-fluoroaniline (800 mg, 2.608 mmol, 1 equiv) and TEA (1.32 g, 13.040 mmol, 5.0 equiv) in DCM (8 mL) was added N-ethyl-N-methylsulfamoyl chloride (2.46 g, 15.648 mmol, 6.0 equiv) at room temperature. The resulting mixture was stirred for 3 h at 70° C. The resulting mixture was concentrated under reduced pressure. The residue was purified by silica gel column chromatography eluted with CH₂Cl₂:MeOH (10:1) to afford ({3-[5-(2-chloropyrimidin-4-yl)-1,3-thiazol-4-yl]-2-fluorophenyl}sulfamoyl)(ethyl)methylamine (290 mg, 25.99%) as a yellow solid. LCMS: C₁₆H₁₅ClFN₅O₂S₂ requires: 427.0, found: m/z=428.0 [M+H]⁺.

### Step-5: Synthesis of tert-butyl 4-[6-({4-[4-(3-{[ethyl(methyl)sulfamoyl]amino}-2-fluorophenyl)-1,3-thiazol-5-yl]pyrimidin-2-yl}amino)pyridin-3-yl]piperazine-1-carboxylate (6)

To a mixture of ({3-[5-(2-chloropyrimidin-4-yl)-1,3-thiazol-4-yl]-2-fluorophenyl}sulfamoyl)(ethyl)methylamine (290 mg, 0.678 mmol, 1 equiv) and tert-butyl 4-(6-aminopyridin-3-yl)piperazine-1-carboxylate (245.24 mg, 0.881 mmol, 1.3 equiv) in 1,4-dioxane (3 mL) was added Cs₂CO₃ (441.63 mg, 1.356 mmol, 2.0 equiv) and BrettPhos Pd G₃ (61.44 mg, 0.068 mmol, 0.1 equiv) at room temperature under a nitrogen atmosphere. The resulting mixture was stirred for 2 h at 80° C. under the nitrogen atmosphere. The resulting mixture was concentrated under reduced pressure. The residue was purified by silica gel column chromatography, eluted with CH₂Cl₂:MeOH (8:1) to afford tert-butyl 4-[6-({4-[4-(3-{[ethyl(methyl)sulfamoyl]amino}-2-fluorophenyl)-1,3-thiazol-5-yl]pyrimidin-2-yl}amino)pyridin-3-yl]piperazine-1-carboxylate (200 mg, 44.06%) as a yellow solid. LCMS: C₃₀H₃₆FN₉O₄S₂ requires: 669.2, found: m/z=670.2 [M+H]⁺.

### Step-6: Synthesis of 4-[4-(3-{[ethyl(methyl)sulfamoyl]amino}-2-fluorophenyl)-1,3-thiazol-5-yl]-N-[5-(piperazin-1-yl)pyridin-2-yl]pyrimidin-2-amine; Trifluoroacetic Acid

To a mixture of tert-butyl 4-[6-({4-[4-(3-{[ethyl(methyl)sulfamoyl]amino}-2-fluorophenyl)-1,3-thiazol-5-yl]pyrimidin-2-yl}amino)pyridin-3-yl]piperazine-1-carboxylate (200 mg, 0.299 mmol, 1 equiv) in DCM (2 mL) was added TFA (2 mL) at room temperature. The resulting mixture was stirred for one hour at room temperature. The resulting mixture was concentrated under reduced pressure. The residue was purified by reversed-phase flash chromatography with the following conditions: column, C18 silica gel; mobile phase, MeCN in water (0.1% TFA), 10% to 50% gradient in 10 min; detector, UV 254 nm to afford 4-[4-(3-{[ethyl(methyl)sulfamoyl]amino}-2-fluorophenyl)-1,3-thiazol-5-yl]-N-[5-(piperazin-1-yl)pyridin-2-yl]pyrimidin-2-amine; trifluoroacetic acid salt (160.8 mg, 78.77%) as a yellow solid. LCMS: C₂₅H₂₈FN₉O₂S₂ requires: 569.2, found: m/z=570.2 [M+H]⁺. ¹HNMR (400 MHz, CD₃OD) δ 9.31 (s, 1H), 8.62-8.58 (m, 1H), 8.17-8.13 (m, 1H), 7.79-7.75 (m, 1H), 7.68-7.64 (m, 1H), 7.56 (s, 1H), 7.49-7.47 (m, 1H), 7.39-7.34 (m 1H), 7.01 (d, J=5.6 Hz, 1H), 3.58-3.54 (m, 4H), 3.47-3.43 (m, 4H), 3.33-3.27 (m, 2H), 2.81 (s, 3H), 1.15-1.12 (m, 3H).

### Synthesis of N-{3-[2-tert-butyl-5-(2-{[5-(piperazin-1-yl)pyridin-2-yl]amino}pyrimidin-4-yl)-1,3-thiazol-4-yl]-2-fluorophenyl}propane-1-sulfonamide; Trifluoroacetic Acid Salt

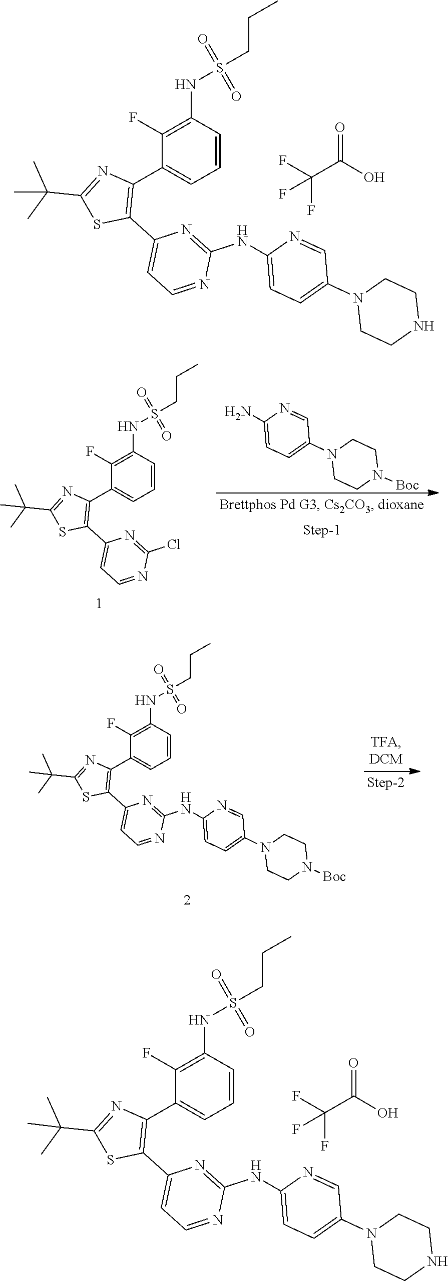

### Step-1: Synthesis of tert-butyl 4-{6-[(4-{2-tert-butyl-4-[2-fluoro-3-(propane-1-sulfonamido)phenyl]-1,3-thiazol-5-yl}pyrimidin-2-yl)amino]pyridin-3-yl}piperazine-1-carboxylate (2)

To a stirred mixture of N-{3-[2-tert-butyl-5-(2-chloropyrimidin-4-yl)-1,3-thiazol-4-yl]-2-fluorophenyl}propane-1-sulfonamide (600 mg, 1.279 mmol, 1 equiv) and tert-butyl 4-(6-aminopyridin-3-yl)piperazine-1-carboxylate (534 mg, 1.918 mmol, 1.5 equiv) in dioxane (10 mL) was added Brettphos Pd G₃ (116 mg, 0.128 mmol, 0.1 equiv) and Cs₂CO₃ (834 mg, 2.558 mmol, 2 equiv). The resulting mixture was stirred at 80° C. for 2 h under a nitrogen atmosphere. The resulting mixture was concentrated under reduced pressure. The residue was purified by silica gel column chromatography eluted with PE:EtOAc (1:4) to afford tert-butyl 4-{6-[(4-{2-tert-butyl-4-[2-fluoro-3-(propane-1-sulfonamido)phenyl]-1,3-thiazol-5-yl}pyrimidin-2-yl)amino]pyridin-3-yl}piperazine-1-carboxylate (620 mg, 61.35%) as a yellow solid. LCMS: C₃₄H₄₃FN₈O₄S₂ requires: 710.3, found: m/z=711.4 [M+H]⁺.

### Step-2: Synthesis of N-{3-[2-tert-butyl-5-(2-{[5-(piperazin-1-yl)pyridin-2-yl]amino}pyrimidin-4-yl)-1,3-thiazol-4-yl]-2-fluorophenyl}propane-1-sulfonamide; Trifluoroacetic Acid

To a stirred mixture of tert-butyl 4-{6-[(4-{2-tert-butyl-4-[2-fluoro-3-(propane-1-sulfonamido)phenyl]-1,3-thiazol-5-yl}pyrimidin-2-yl)amino]pyridin-3-yl}piperazine-1-carboxylate (600 mg, 0.844 mmol, 1 equiv) in DCM (6 mL) was added TFA (3 mL) at 0° C. The resulting mixture was stirred at room temperature for one hour. The resulting mixture was concentrated under reduced pressure. This resulted in N-{3-[2-tert-butyl-5-(2-{[5-(piperazin-1-yl)pyridin-2-yl]amino}pyrimidin-4-yl)-1,3-thiazol-4-yl]-2-fluorophenyl}propane-1-sulfonamide; trifluoroacetic acid salt (549.7 mg, crude) as a brown solid. LCMS: C₂₉H₃₅FN₈O₂S₂ requires: 610.2, found: m/z=611.2 [M+H]⁺. ¹H NMR (400 MHz, DMSO-d₆) δ 10.76-10.72 (m, 1H), 9.72-9.68 (m, 1H), 8.91-8.87 (m, 2H), 7.95-7.90 (m, 1H), 7.81-7.69 (m, 2H), 7.58-7.47 (m, 2H), 7.42-7.34 (m, 1H), 6.75-6.69 (m, 1H), 3.47-3.40 (m, 4H), 3.40-3.31 (m, 4H), 3.03-2.95 (m, 2H), 1.74-1.60 (m, 2H), 1.50-1.46 (m, 9H), 0.96-0.87 (m, 3H).

### Synthesis of N-{2-fluoro-3-[2-methyl-5-(2-{[5-(piperazin-1-yl)pyridin-2-yl]amino}pyrimidin-4-yl)-1,3-thiazol-4-yl]phenyl}propane-1-sulfonamide; Trifluoroacetic Acid Salt

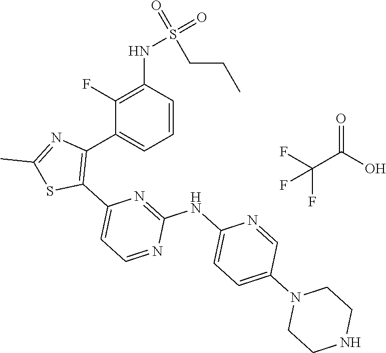 [SMILES: CCCS(=O)(=O)Nc1cccc(-c2nc(C)sc2-c2ccnc(Nc3ccc(N4CCNCC4)cn3)n2)c1F ; O=C(O)C(F)(F)F]

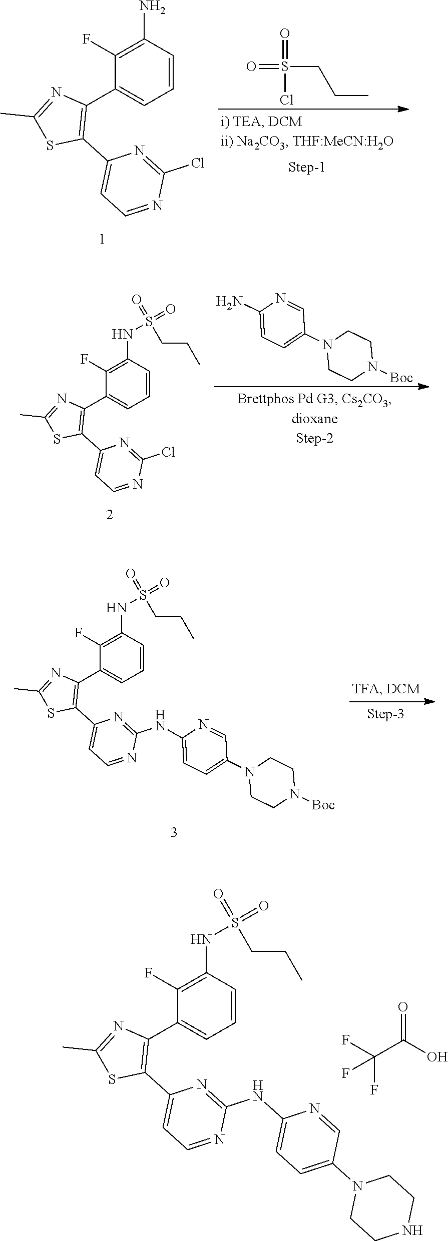

### Step-1: Synthesis of N-{3-[5-(2-chloropyrimidin-4-yl)-2-methyl-1,3-thiazol-4-yl]-2-fluorophenyl}propane-1-sulfonamide (2)

To a stirred mixture of 3-[5-(2-chloropyrimidin-4-yl)-2-methyl-1,3-thiazol-4-yl]-2-fluoroaniline (600 mg, 1.870 mmol, 1 equiv) and TEA (568 mg, 5.61 mmol, 3 equiv) in DCM (10 mL) was added propane-1-sulfonyl chloride (800 mg, 5.61 mmol, 3 equiv) at 0° C. The resulting mixture was stirred at room temperature for one hour. The resulting mixture was concentrated under reduced pressure. To the above mixture was added THF (5 mL), MeCN (5 mL), and Na₂CO₃ (2.97 g, 28.05 mmol, 15 equiv) in H₂O (5 mL). The resulting mixture was stirred at 50° C. overnight. The resulting mixture was concentrated under reduced pressure. The residue was purified by silica gel column chromatography eluted with PE:EtOAc (2:3) to afford N-{3-[5-(2-chloropyrimidin-4-yl)-2-methyl-1,3-thiazol-4-yl]-2-fluorophenyl}propane-1-sulfonamide (550 mg, 61.99%) as a brown solid. LCMS: C₁₇H₁₆ClFN₄O₂S₂ requires: 426.0, found: m/z=427.0 [M+H]⁺.

### Step-2: Synthesis of tert-butyl 4-{6-[(4-{4-[2-fluoro-3-(propane-1-sulfonamido)phenyl]-2-methyl-1,3-thiazol-5-yl}pyrimidin-2-yl)amino]pyridin-3-yl}piperazine-1-carboxylate (3)

To a stirred mixture of N-{3-[5-(2-chloropyrimidin-4-yl)-2-methyl-1,3-thiazol-4-yl]-2-fluorophenyl}propane-1-sulfonamide (550 mg, 1.288 mmol, 1 equiv) and tert-butyl 4-(6-aminopyridin-3-yl)piperazine-1-carboxylate (538 mg, 1.932 mmol, 1.5 equiv) in dioxane (10 mL) was added Brettphos Pd G3 (117 mg, 0.129 mmol, 0.1 equiv) and Cs₂CO₃ (839.52 mg, 2.576 mmol, 2 equiv). The resulting mixture was stirred at 80° C. for one hour under a nitrogen atmosphere. The resulting mixture was concentrated under reduced pressure. The residue was purified by silica gel column chromatography eluted with PE:EtOAc (1:4) to afford tert-butyl 4-{6-[(4-{4-[2-fluoro-3-(propane-1-sulfonamido)phenyl]-2-methyl-1,3-thiazol-5-yl}pyrimidin-2-yl)amino]pyridin-3-yl}piperazine-1-carboxylate (600 mg, 66.15%) as a brown solid. LCMS: C₃₁H₃₇FN₈O₄S₂ requires: 668.2, found: m/z=669.2 [M+H]⁺.

### Step-3: Synthesis of in N-{2-fluoro-3-[2-methyl-5-(2-{[5-(piperazin-1-yl)pyridin-2-yl]amino}pyrimidin-4-yl)-1,3-thiazol-4-yl]phenyl}propane-1-sulfonamide; Trifluoroacetic Acid

To a stirred mixture of tert-butyl 4-{6-[(4-{4-[2-fluoro-3-(propane-1-sulfonamido)phenyl]-2-methyl-1,3-thiazol-5-yl}pyrimidin-2-yl)amino]pyridin-3-yl}piperazine-1-carboxylate (300 mg, 0.449 mmol, 1 equiv) in DCM (8 mL) was added TFA (2 mL) at 0° C. The resulting mixture was stirred at room temperature for one hour. The resulting mixture was concentrated under reduced pressure. This resulted in N-{2-fluoro-3-[2-methyl-5-(2-{[5-(piperazin-1-yl)pyridin-2-yl]amino}pyrimidin-4-yl)-1,3-thiazol-4-yl]phenyl}propane-1-sulfonamide; trifluoroacetic acid salt (225.1 mg, crude) as a brown solid. LCMS: C₂₆H₂₉FN₈O₂S₂ requires: 568.2, found: m/z=569.3 [M+H]⁺. ¹H NMR (400 MHz, CD₃OD) δ 8.60-8.54 (m, 1H), 8.23-8.16 (m, 1H), 7.81-7.76 (m, 1H), 7.68-7.59 (m, 1H), 7.56-7.47 (m, 2H), 7.43-7.35 (m, 1H), 7.00-6.94 (m, 1H), 3.56-3.51 (m, 4H), 3.50-3.42 (m, 4H), 3.13-3.05 (m, 2H), 2.86-2.79 (m, 3H), 1.92-1.76 (m, 2H), 1.07-0.96 (m, 3H).

### Synthesis of 4-[2-tert-butyl-4-(3-{[ethyl(methyl)sulfamoyl]amino}-2-fluorophenyl)-1,3-thiazol-5-yl]-N-(5-{3,8-diazabicyclo[3.2.1]octan-8-yl}pyridin-2-yl)pyrimidin-2-amine; Trifluoroacetic Acid Salt

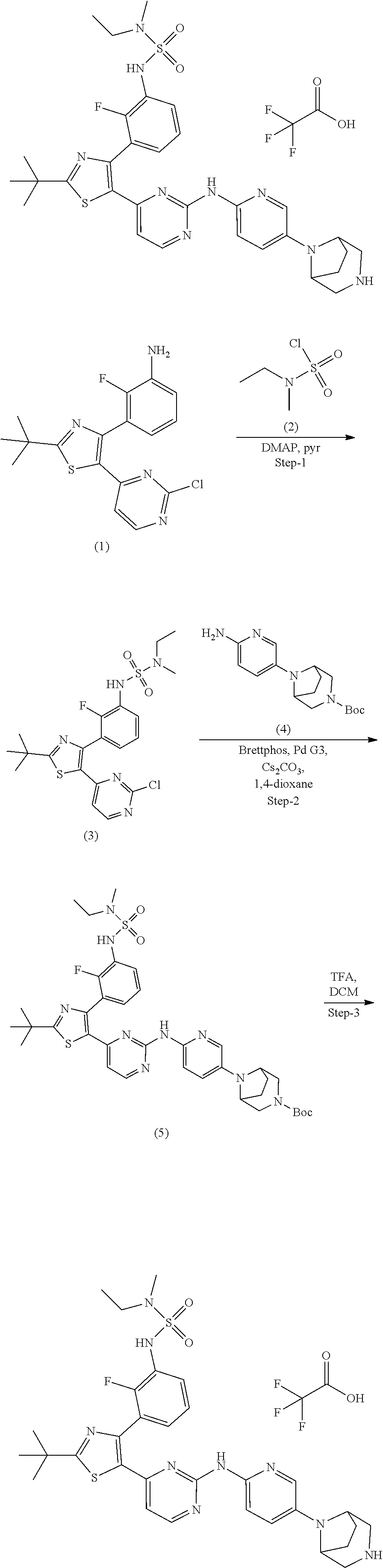

### Step-1: Synthesis of (13-[2-tert-butyl-5-(2-chloropyrimidin-4-yl)-1,3-thiazol-4-yl]-2-fluorophenyl]sulfamoyl)(ethyl)methylamine (3)

To a mixture of 3-[2-tert-butyl-5-(2-chloropyrimidin-4-yl)-1,3-thiazol-4-yl]-2-fluoroaniline (3.0 g, 8.268 mmol, 1 equiv) and DMAP (1.21 g, 9.922 mmol, 1.2 equiv) in pyridine (1.5 mL) was added N-ethyl-N-methylsulfamoyl chloride (2.61 g, 16.536 mmol, 2.0 equiv) dropwise at room temperature. The resulting mixture was stirred overnight at 40° C. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with DCM:EtOAc (5:1) to afford ({3-[2-tert-butyl-5-(2-chloropyrimidin-4-yl)-1,3-thiazol-4-yl]-2-fluorophenyl}sulfamoyl)(ethyl)methylamine (1.82 g, 45.48%) as a yellow solid. LCMS: C₂₀H₂₃ClFN₅O₂S₂ requires: 483.1, found: m/z=484.1 [M+H]⁺.

### Step-2: Synthesis of tert-butyl 8-[6-({4-[2-tert-butyl-4-(3-{[ethyl(methyl)sulfamoyl]amino}-2-fluorophenyl)-1,3-thiazol-5-yl]pyrimidin-2-yl}amino)pyridin-3-yl]-3,8-diazabicyclo[3.2.1]octane-3-carboxylate (5)

To a mixture of ({3-[2-tert-butyl-5-(2-chloropyrimidin-4-yl)-1,3-thiazol-4-yl]-2-fluorophenyl}sulfamoyl)(ethyl)methylamine (700 mg, 1.446 mmol, 1 equiv) and tert-butyl 8-(6-aminopyridin-3-yl)-3,8-diazabicyclo[3.2.1]octane-3-carboxylate (572.30 mg, 1.880 mmol, 1.3 equiv) in 1,4-dioxane (6 mL) was added BrettPhos Pd G₃ (131.10 mg, 0.145 mmol, 0.1 equiv) and Cs₂CO₃ (942.43 mg, 2.892 mmol, 2.0 equiv) at room temperature. The resulting mixture was stirred for 2 h at 80° C. under a nitrogen atmosphere. The resulting mixture was concentrated under reduced pressure. The residue was purified by silica gel column chromatography eluted with CH₂Cl₂:MeOH (5:1) to afford tert-butyl 8-[6-({4-[2-tert-butyl-4-(3-{[ethyl(methyl)sulfamoyl]amino}-2-fluorophenyl)-1,3-thiazol-5-yl]pyrimidin-2-yl}amino)pyridin-3-yl]-3,8-diazabicyclo[3.2.1]octane-3-carboxylate (400 mg, 36.78%) as a yellow solid. LCMS: C₃₆H₄₆FN₉O₄S₂ requires: 751.3, found: m/z=752.3 [M+H]⁺.

### Step-3: Synthesis of 4-[2-tert-butyl-4-(3-{[ethyl(methyl)sulfamoyl]amino}-2-fluorophenyl)-1,3-thiazol-5-yl]-N-(5-{3,8-diazabicyclo[3.2.1]octan-8-yl}pyridin-2-yl)pyrimidin-2-amine; Trifluoroacetic Acid

To a stirred mixture of tert-butyl 8-[6-({4-[2-tert-butyl-4-(3-{[ethyl(methyl)sulfamoyl]amino}-2-fluorophenyl)-1,3-thiazol-5-yl]pyrimidin-2-yl}amino)pyridin-3-yl]-3,8-diazabicyclo[3.2.1]octane-3-carboxylate (390 mg, 0.519 mmol, 1 equiv) in DCM (2 mL) was added TFA (2 mL) dropwise at 0° C. The resulting mixture was stirred for one hour at room temperature. The resulting mixture was concentrated under reduced pressure. The residue was purified by reversed-phase flash chromatography with the following conditions: column, C18 silica gel; mobile phase, MeCN in water (0.1% TFA), 10% to 50% gradient in 10 min; detector, UV 254 nm to afford 4-[2-tert-butyl-4-(3-{[ethyl(methyl)sulfamoyl]amino}-2-fluorophenyl)-1,3-thiazol-5-yl]-N-(5-{3,8-diazabicyclo[3.2.1]octan-8-yl}pyridin-2-yl)pyrimidin-2-amine; trifluoroacetic acid salt (352.1 mg, 86.25%) as a yellow solid. LCMS: C₃₁H₃₈FN₉O₂S₂ requires: 651.3, found: m/z=652.3 [M+H]⁺. ¹H NMR (300 MHz, CD₃OD) δ 8.57-8.55 (m, 1H), 8.07 (d, J=9.6 Hz, 1H), 7.72 (s, 1H), 7.62 (d, J=8.1 Hz, 1H), 7.52-7.48 (m, 2H), 7.42-7.31 (m, 1H), 6.96 (d, J=4.8 Hz, 1H), 4.57-4.43 (m, 2H), 3.52-3.38 (m, 2H), 3.31-3.18 (m, 4H), 2.85-2.79 (m, 3H), 2.41-2.23 (m, 2H), 2.21-2.11 (m, 2H), 1.62-1.48 (m, 9H), 1.20-1.08 (m, 3H).

### Synthesis of 4-{4-[4-(3-{[ethyl(methyl)sulfamoyl]amino}-2-fluorophenyl)-3-(pyrimidin-4-yl)pyrazol-1-yl]phenyl}piperazine; Trifluoroacetic Acid Salt

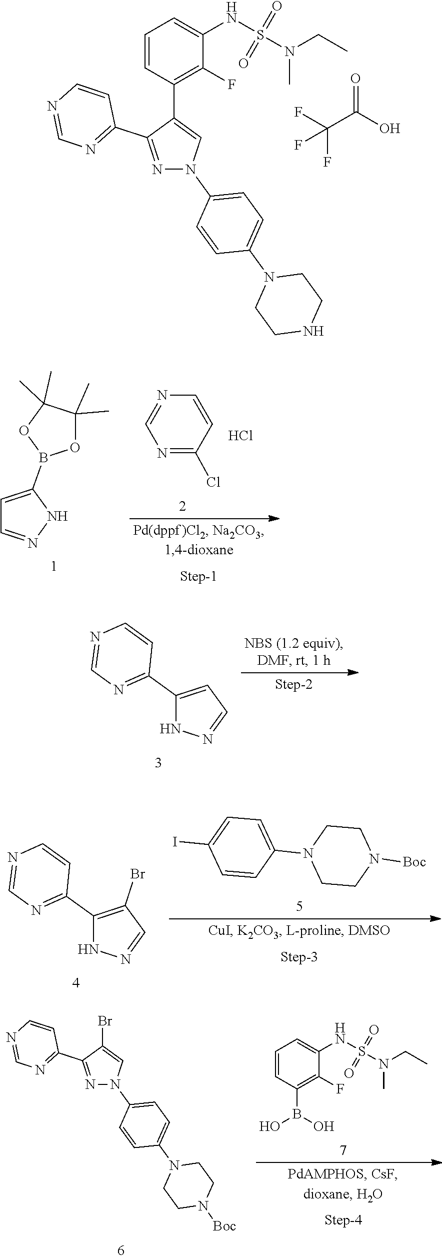

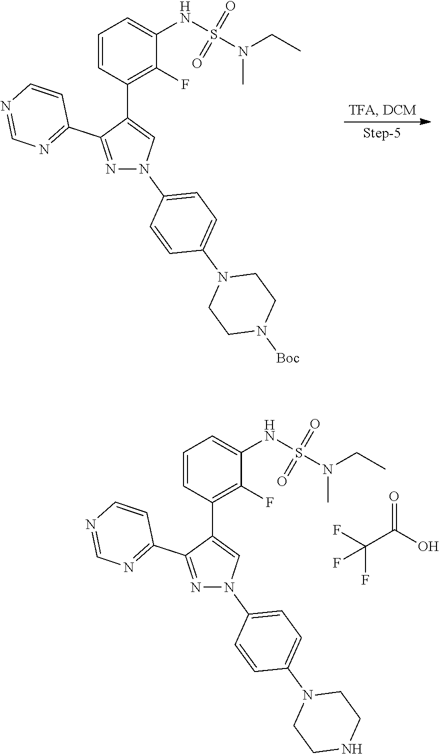

### Step-1: Synthesis of 4-(2H-pyrazol-3-yl)pyrimidine (3)

To a mixture of 3-(4,4,5,5-tetramethyl-1,3,2-dioxaborolan-2-yl)-2H-pyrazole (3.05 g, 15.716 mmol, 1.2 equiv) and 4-chloropyrimidine (1.5 g, 13.097 mmol, 1.00 equiv) in 1,4-dioxane (20 mL) and H₂O (2 mL) was added Na₂CO₃ (2.78 g, 26.194 mmol, 2.0 equiv) and Pd(dppf)Cl₂ (958.32 mg, 1.310 mmol, 0.1 equiv) at room temperature. The resulting mixture was stirred overnight at 100° C. under a nitrogen atmosphere. The resulting mixture was concentrated under reduced pressure. The residue was purified by silica gel column chromatography eluted with CH₂Cl₂:MeOH (5:1) to afford 4-(2H-pyrazol-3-yl)pyrimidine (790 mg, 41.27%) as a yellow solid. LCMS: C₇H₆N₄ requires: 146.1, found: m/z=147.1 [M+H]⁺.

### Step-2: Synthesis of 4-(4-bromo-2H-pyrazol-3-yl)pyrimidine (4)

To a mixture of 4-(2H-pyrazol-3-yl)pyrimidine (790 mg, 5.405 mmol, 1 equiv) in DMF (8 mL) was added NBS (1.06 g, 5.946 mmol, 1.1 equiv) in portions at 0° C. The resulting mixture was stirred for one hour at room temperature. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with CH₂Cl₂:MeOH (5:1) to afford 4-(4-bromo-2H-pyrazol-3-yl)pyrimidine (710 mg, 58.37%) as a yellow solid. LCMS: C₇H₅BrN₄ requires: 224.0, found: m/z=225.0 [M+H]⁺.

### Step-3: Synthesis of tert-butyl 4-{4-[4-bromo-3-(pyrimidin-4-yl)pyrazol-1-yl]phenyl}piperazine-1-carboxylate (6)

To a stirred mixture of 4-(4-bromo-2H-pyrazol-3-yl)pyrimidine (710 mg, 3.155 mmol, 1 equiv) and tert-butyl 4-(4-iodophenyl)piperazine-1-carboxylate (1.83 g, 4.732 mmol, 1.5 equiv) in DMSO (8 mL) was added L-proline (145.29 mg, 1.262 mmol, 0.4 equiv), K₂CO₃ (1.31 g, 9.47 mmol, 3.0 equiv), and CuI (120.17 mg, 0.631 mmol, 0.2 equiv) at room temperature. The resulting mixture was stirred for 2 h at 100° C. under a nitrogen atmosphere. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with CH₂Cl₂:MeOH (5:1) to afford tert-butyl 4-{4-[4-bromo-3-(pyrimidin-4-yl)pyrazol-1-yl]phenyl}piperazine-1-carboxylate (310 mg, 20.24%) as a yellow solid. LCMS: C₂₂H₂₅BrN₆O₂ requires: 484.1, found: m/z=485.1 [M+H]⁺.

### Step-4: Synthesis of tert-butyl 4-{4-[4-(3-{[ethyl(methyl)sulfamoyl]amino}-2-fluorophenyl)-3-(pyrimidin-4-yl)pyrazol-1-yl]phenyl}piperazine-1-carboxylate

To a mixture of tert-butyl 4-{4-[4-bromo-3-(pyrimidin-4-yl)pyrazol-1-yl]phenyl}piperazine-1-carboxylate (310 mg, 0.639 mmol, 1 equiv) and 3-{[ethyl(methyl)sulfamoyl]amino}-2-fluorophenylboronic acid (176.33 mg, 0.639 mmol, 1.0 equiv) in 1,4-dioxane (3.0 mL) and H₂O (0.3 mL) was added CsF (194.03 mg, 1.278 mmol, 2.0 equiv) and PdCl₂(AMPHOS)₂ (90.45 mg, 0.128 mmol, 0.2 equiv) at room temperature under a nitrogen atmosphere. The resulting mixture was stirred for 2 h at 90° C. under the nitrogen atmosphere. The resulting mixture was concentrated under reduced pressure. The residue was purified by silica gel column chromatography eluted with CH₂Cl₂:CH₃OH (5:1) to afford tert-butyl 4-{4-[4-(3-{[ethyl(methyl)sulfamoyl]amino}-2-fluorophenyl)-3-(pyrimidin-4-yl)pyrazol-1-yl]phenyl}piperazine-1-carboxylate (150 mg, 36.88%) as a pink solid. LCMS: C₃₁H₃₇FN₈O₄S requires: 636.3, found: m/z=637.2 [M+H]⁺. ¹H NMR (400 MHz, CD₃OD) δ 9.06 (s, 1H), 8.68 (d, J=5.2 Hz, 1H), 7.93 (s, 1H), 7.52-7.44 (m, 1H), 7.28-7.22 (m, 1H), 7.20 (d, J=8.8 Hz, 2H), 7.18-7.10 (m, 2H), 7.00 (d, J=8.9 Hz, 2H), 3.59-3.54 (m, 4H), 3.35-3.26 (m, 4H), 3.21-3.12 (m, 2H), 2.74 (s, 3H), 1.50 (s, 9H), 1.07-1.01 (m, 3H).

### Step-5: Synthesis of 4-{4-[4-(3-{[ethyl(methyl)sulfamoyl]amino}-2-fluorophenyl)-3-(pyrimidin-4-yl)pyrazol-1-yl]phenyl}piperazine; Trifluoroacetic Acid

To a mixture of tert-butyl 4-{4-[4-(3-{[ethyl(methyl)sulfamoyl]amino}-2-fluorophenyl)-3-(pyrimidin-4-yl)pyrazol-1-yl]phenyl}piperazine-1-carboxylate (150 mg, 0.236 mmol, 1 equiv) in DCM (3 mL) was added TFA (3 mL) at room temperature. The resulting mixture was stirred for one hour at room temperature. The resulting mixture was concentrated under reduced pressure. The residue was purified by reversed-phase flash chromatography with the following conditions: column, C18 silica gel; mobile phase, MeCN in water (0.1% TFA), 10% to 60% gradient in 10 min; detector, UV 254 nm to afford 4-{4-[4-(3-{[ethyl(methyl)sulfamoyl]amino}-2-fluorophenyl)-3-(pyrimidin-4-yl)pyrazol-1-yl]phenyl}piperazine; trifluoroacetic acid salt (130.8 mg, 85.34%) as a yellow solid. LCMS: C₂₆H₂₉FN₈O₂S requires: 536.2, found: m/z=537.4 [M+H]⁺. ¹H NMR (400 MHz, CD₃OD) δ 9.02-8.97 (m, 1H), 8.81-8.75 (m, 1H), 8.43 (s, 1H), 8.01-7.95 (m, 1H), 7.92-7.84 (m, 2H), 7.60-7.51 (m, 1H), 7.31-7.23 (m, 1H), 7.26-7.15 (m, 3H), 3.56-3.49 (m, 4H), 3.48-3.40 (m, 4H), 3.26-3.16 (m, 2H), 2.81 (s, 3H), 1.15-1.07 (m, 3H).

### Synthesis of 4-{4-[3-(3-{[ethyl(methyl)sulfamoyl]amino}-2-fluorophenyl)-4-(pyridin-4-yl)pyrazol-1-yl]phenyl}piperidine; Trifluoroacetic Acid Salt

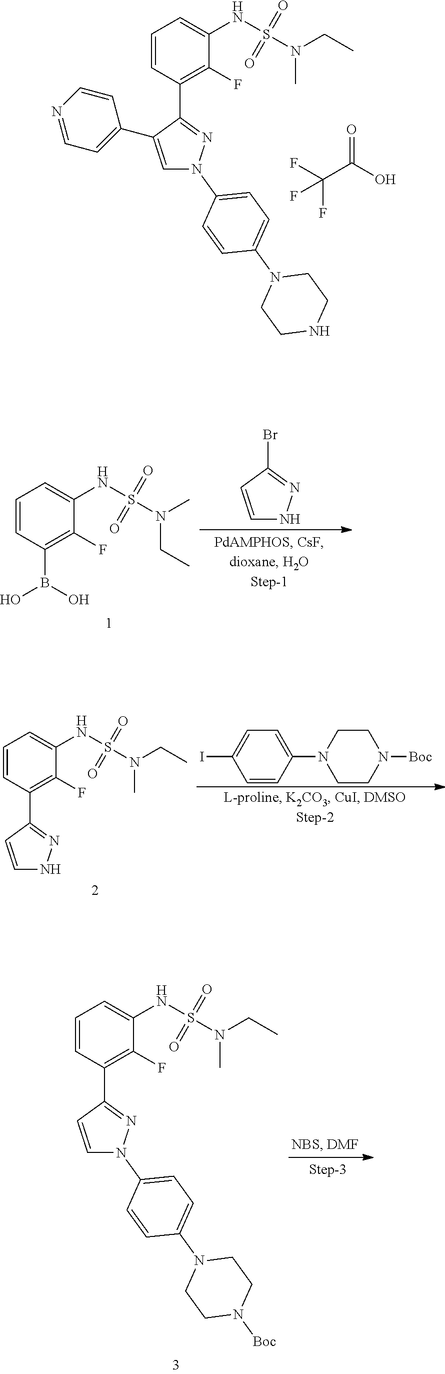

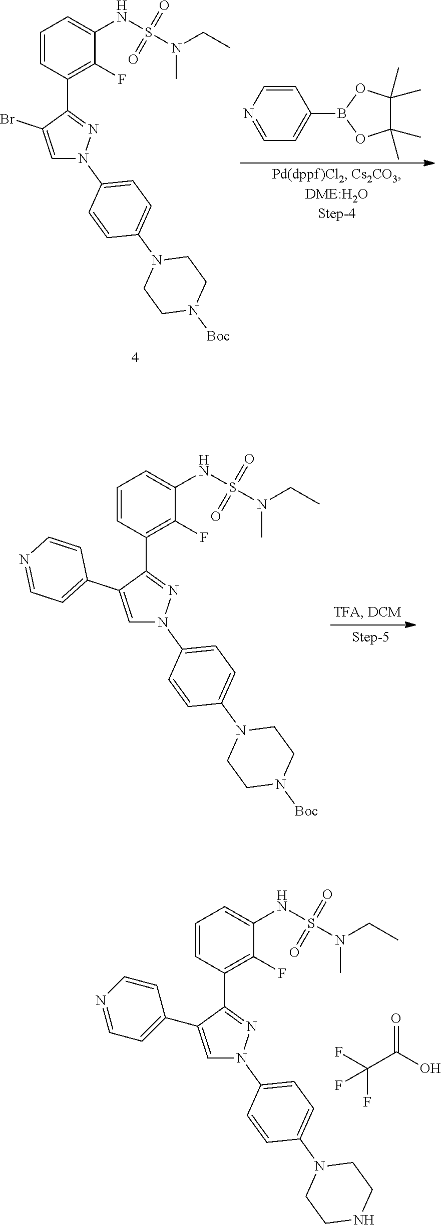

### Step-1: Synthesis of 3-{[ethyl(methyl)sulfamoyl]amino}-2-fluorophenylpyrazole (2)

To a mixture of 3-bromo-1H-pyrazole (1.6 g, 10.886 mmol, 1.2 equiv) and 3-{[ethyl(methyl)sulfamoyl]amino}-2-fluorophenylboronic acid (2.5 g, 9.055 mmol, 1 equiv) in 1,4-dioxane (30 mL) was added CsF (2.75 g, 18.104 mmol, 2 equiv) in H₂O (6 mL) and PdAMPHOS (1.28 g, 1.811 mmol, 0.2 equiv). The resulting mixture was stirred at 90° C. for 2 h under a nitrogen atmosphere. The resulting mixture was concentrated under vacuum. The residue was purified by silica gel column chromatography eluted with PE:EtOAc (1/2) to afford 3-{[ethyl(methyl)sulfamoyl]amino}-2-fluorophenylpyrazole (1.9 g, 63.30%) as a yellow solid. LCMS: C₁₂H₁₅FN₄O₂S requires: 298.1, found: m/z=299.1 [M+H]⁺.

### Step-2: Synthesis of tert-butyl 4-{4-[3-(3-{[ethyl(methyl)sulfamoyl]amino}-2-fluorophenyl)pyrazol-1-yl]phenyl}piperazine-1-carboxylate (3)

To a mixture of 3-{[ethyl(methyl)sulfamoyl]amino}-2-fluorophenylpyrazole (800 mg, 2.682 mmol, 1 equiv) and tert-butyl 4-(4-iodophenyl)piperazine-1-carboxylate (1.56 g, 4.018 mmol, 1.50 equiv) in DMSO (20 mL) was added L-proline (123 mg, 1.073 mmol, 0.4 equiv), K₂CO₃(1.11 g, 8.046 mmol, 3 equiv), and CuI (102 mg, 0.536 mmol, 0.2 equiv). The resulting mixture was stirred at 100° C. for three days under a nitrogen atmosphere. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with PE:EtOAc (1:1) to afford tert-butyl 4-{4-[3-(3-{[ethyl(methyl)sulfamoyl]amino}-2-fluorophenyl)pyrazol-1-yl]phenyl}piperazine-1-carboxylate (630 mg, 37.85%) as a yellow solid. LCMS: C₂₇H₃₅FN₆O₄S requires: 558.2, found: m/z=559.4 [M+H]⁺.

### Step-3: Synthesis of tert-butyl 4-{4-[4-bromo-3-(3-{[ethyl(methyl)sulfamoyl]amino}-2-fluorophenyl)pyrazol-1-yl]phenyl}piperazine-1-carboxylate (4)

To a mixture of tert-butyl 4-{4-[3-(3-{[ethyl(methyl)sulfamoyl]amino}-2-fluorophenyl)pyrazol-1-yl]phenyl}piperazine-1-carboxylate (620 mg, 1.11 mmol, 1 equiv) in DMF (5 mL) was added NBS (277 mg, 1.554 mmol, 1.4 equiv) at 0° C. The resulting mixture was stirred at room temperature for one hour. The resulting mixture was extracted with EtOAc. The combined organic layers were washed with saturated aqueous Na₂S₂O₃ and dried over anhydrous Na₂SO₄. After filtration, the filtrate was concentrated under reduced pressure. This resulted in tert-butyl 4-{4-[4-bromo-3-(3-{[ethyl(methyl)sulfamoyl]amino}-2-fluorophenyl)pyrazol-1-yl]phenyl}piperazine-1-carboxylate (700 mg, 98.93%) as a yellow solid. LCMS: C₂₇H₃₄BrFN₆O₄S requires: 636.2, found: m/z=637.4 [M+H]⁺.

### Step-4: Synthesis of tert-butyl 4-{4-[3-(3-{[ethyl(methyl)sulfamoyl]amino}-2-fluorophenyl)-4-(pyridin-4-yl)pyrazol-1-yl]phenyl}piperazine-1-carboxylate

To a mixture of tert-butyl 4-{4-[4-bromo-3-(3-{[ethyl(methyl)sulfamoyl]amino}-2-fluorophenyl)pyrazol-1-yl]phenyl}piperazine-1-carboxylate (700 mg, 1.098 mmol, 1 equiv) and 4-(4,4,5,5-tetramethyl-1,3,2-dioxaborolan-2-yl)pyridine (450 mg, 2.196 mmol, 2 equiv) in DME (11 mL) was added Cs₂CO₃ (715 mg, 2.196 mmol, 2 equiv) in H₂O (1.3 mL) and Pd(dppf)Cl₂CH₂Cl₂ (89 mg, 0.11 mmol, 0.1 equiv). The final reaction mixture was irradiated with microwave radiation at 100° C. for 30 min under a nitrogen atmosphere. The resulting mixture was concentrated under reduced pressure. The residue was purified by silica gel column chromatography eluted with PE:EtOAc (1:1) to afford tert-butyl 4-{4-[3-(3-{[ethyl(methyl)sulfamoyl]amino}-2-fluorophenyl)-4-(pyridin-4-yl)pyrazol-1-yl]phenyl}piperazine-1-carboxylate (420 mg, 57.16%) as a yellow solid. LCMS: C₃₂H₃₈FN₇O₄S requires: 635.3, found: m/z=636.4 [M+H]⁺. ¹H NMR (400 MHz, CD₃OD) δ 8.68-8.62 (m, 2H), 8.35-8.30 (m, 1H), 7.91-7.80 (m, 5H), 7.57-7.49 (m, 1H), 7.34 (d, J=8.7 Hz, 1H), 7.26-7.17 (m, 1H), 6.96-6.90 (m, 1H), 3.42-3.35 (m, 4H), 3.29-3.19 (m, 2H), 2.89-2.82 (m, 7H), 1.47 (s, 9H), 1.14-1.06 (m, 3H).

### Step-5: Synthesis of 4-{4-[3-(3-{[ethyl(methyl)sulfamoyl]amino}-2-fluorophenyl)-4-(pyridin-4-yl)pyrazol-1-yl]phenyl}piperidine; Trifluoroacetic Acid

To a mixture of tert-butyl 4-{4-[3-(3-{[ethyl(methyl)sulfamoyl]amino}-2-fluorophenyl)-4-(pyridin-4-yl)pyrazol-1-yl]phenyl}piperazine-1-carboxylate (420 mg, 0.661 mmol, 1 equiv) in DCM (9 mL) was added TFA (3 mL) at 0° C. The resulting mixture was stirred at 0° C. for one hour. The resulting mixture was concentrated under vacuum. This resulted in 4-{4-[3-(3-{[ethyl(methyl)sulfamoyl]amino}-2-fluorophenyl)-4-(pyridin-4-yl)pyrazol-1-yl]phenyl}piperidine; trifluoroacetic acid salt (411.2 mg, crude) as a yellow solid. LCMS: C₂₇H₃₀FN₇O₂S requires: 535.2, found: m/z=536.2 [M+H]⁺. ¹H NMR (400 MHz, CD₃OD) δ 8.96-8.87 (m, 2H), 8.54-8.44 (m, 2H), 8.39 (d, J=2.8 Hz, 1H), 8.10-8.01 (m, 2H), 7.89-7.79 (m, 1H), 7.58-7.46 (m, 2H), 7.25-7.12 (m, 1H), 7.01-6.89 (m, 1H), 3.36-3.28 (m, 6H), 3.20-3.11 (m, 4H), 2.82 (s, 3H), 1.12-1.04 (m, 3H).

### General Procedure for Buchwald Coupling Followed by Deprotection

### Synthesis of N-{3-[2-tert-butyl-5-(2-{[4-(piperazin-1-ylmethyl)phenyl]amino}pyrimidin-4-yl)-1,3-thiazol-4-yl]-2-fluorophenyl}propane-1-sulfonamide

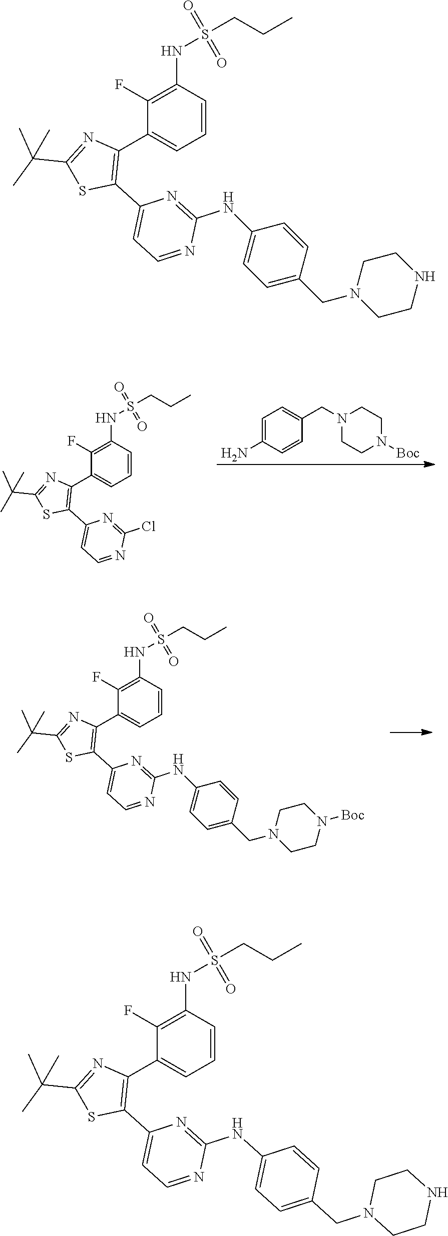 [SMILES: CCCS(=O)(=O)Nc1cccc(-c2nc(C(C)(C)C)sc2-c2ccnc(Nc3ccc(CN4CCNCC4)cc3)n2)c1F ; CCCS(=O)(=O)Nc1cccc(-c2nc(C(C)(C)C)sc2-c2ccnc(Cl)n2)c1F ; CC(C)(C)OC(=O)N1CCN(Cc2ccc(N)cc2)CC1 ; CCCS(=O)(=O)Nc1cccc(-c2nc(C(C)(C)C)sc2-c2ccnc(Nc3ccc(CN4CCN(C(=O)OC(C)(C)C)CC4)cc3)n2)c1F ; CCCS(=O)(=O)Nc1cccc(-c2nc(C(C)(C)C)sc2-c2ccnc(Nc3ccc(CN4CCNCC4)cc3)n2)c1F]

### Step-1: tert-butyl 4-(4-((4-(2-(tert-butyl)-4-(2-fluoro-3-(propylsulfonamido)phenyl)thiazol-5-yl)pyrimidin-2-yl)amino)benzyl)piperazine-1-carboxylate

To a solution of N-{3-[2-tert-butyl-5-(2-chloropyrimidin-4-yl)-1,3-thiazol-4-yl]-2-fluorophenyl}propane-1-sulfonamide (703 mg, 1.5 mmol) and tert-butyl 4-(4-aminobenzyl)piperazine-1-carboxylate (304 mg, 1.05 equiv) in dioxane (10.00 mL) was added sodium tert-butoxide (216 mg, 1.5 eq), Pd₂(dba)₃ or tris(dibenzylideneacetone)dipalladium(0) (205 mg, 0.15 equiv), and 2′-(dicyclohexylphosphanyl)-N,N-dimethyl-[1,1′-biphenyl]-2-amine (DavePhos 177 mg, 0.3 mmol). Then the mixture was purged with nitrogen gas for 5 min, followed by irradiation in a microwave reactor at 120° C. for 2 h. The reaction mixture was filtered through a Celite plug and washed with ethyl acetate. Solvent was then removed by rotary evaporation. Silica gel column chromatography purification eluting with 0-70% EtOAc:DCM provided tert-butyl 4-(4-((4-(2-(tert-butyl)-4-(2-fluoro-3-(propylsulfonamido)phenyl)thiazol-5-yl)pyrimidin-2-yl)amino)benzyl)piperazine-1-carboxylate.

### Step-2: N-{3-[2-tert-butyl-5-(2-{[4-(piperazin-1-ylmethyl)phenyl]amino}pyrimidin-4-yl)-1,3-thiazol-4-yl]-2-fluorophenyl}propane-1-sulfonamide

Tert-butyl 4-(4-((4-(2-(tert-butyl)-4-(2-fluoro-3-(propylsulfonamido)phenyl)thiazol-5-yl)pyrimidin-2-yl)amino)benzyl)piperazine-1-carboxylate was dissolved in DCM:TFA (4:1 ratio, 0.1 M) at room temperature, followed by stirring for 3 h. The reaction mixture was concentrated, then triturated with ether, and used as-is (667 mg, 63% over two steps). LCMS: C₃₁H₃₈FN₇O₂S₂ requires 623.3, found: m/z=624.5 [M+H]⁺.

The following compounds were made using the above General Procedure for Buchwald Coupling Followed by Deprotection.

### Tert-butyl 4-[(4-{2-tert-butyl-4-[2-fluoro-3-(propane-1-sulfonamido)phenyl]-1,3-thiazol-5-yl}pyrimidin-2-yl)amino]benzoate

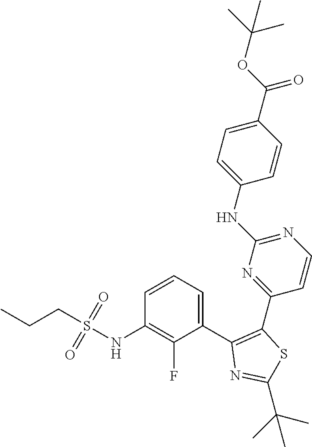 [SMILES: CCCS(=O)(=O)Nc1cccc(-c2nc(C(C)(C)C)sc2-c2ccnc(Nc3ccc(C(=O)OC(C)(C)C)cc3)n2)c1F]

LCMS: C₃₁H₃₆FN₅O₄S₂ requires 625.2, found: m/z=626.5 [M+H]⁺.

### 4-[(4-{2-tert-butyl-4-[2-fluoro-3-(propane-1-sulfonamido)phenyl]-1,3-thiazol-5-yl}pyrimidin-2-yl)amino]benzoic Acid

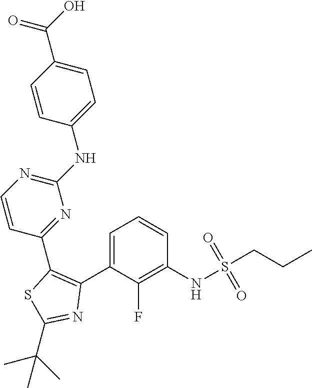 [SMILES: CCCS(=O)(=O)Nc1cccc(-c2nc(C(C)(C)C)sc2-c2ccnc(Nc3ccc(C(=O)O)cc3)n2)c1F]

LCMS: C₂₇H₂₈FN₅O₄S₂ requires 569.2, found: m/z=570.5 [M+H]⁺.

### Tert-butyl 4-{4-[(4-{2-tert-butyl-4-[2-fluoro-3-(propane-1-sulfonamido)phenyl]-1,3-thiazol-5-yl}pyrimidin-2-yl)amino]-2-fluorophenyl}piperazine-1-carboxylate

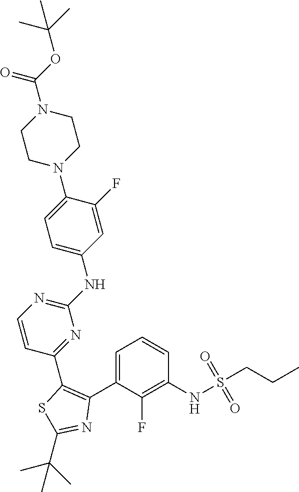 [SMILES: CCCS(=O)(=O)Nc1cccc(-c2nc(C(C)(C)C)sc2-c2ccnc(Nc3ccc(N4CCN(C(=O)OC(C)(C)C)CC4)c(F)c3)n2)c1F]

LCMS: C₃₅H₄₃F₂N₇O₄S₂ requires 727.3, found: m/z=728.5 [M+H]⁺.

### N-{3-[2-tert-butyl-5-(2-{[3-fluoro-4-(piperazin-1-yl)phenyl]amino}pyrimidin-4-yl)-1,3-thiazol-4-yl]-2-fluorophenyl}propane-1-sulfonamide

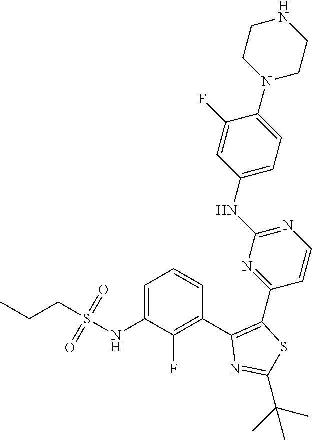 [SMILES: CCCS(=O)(=O)Nc1cccc(-c2nc(C(C)(C)C)sc2-c2ccnc(Nc3ccc(N4CCNCC4)c(F)c3)n2)c1F]

LCMS: C₃₅H₄₃F₂N₇O₄S₂ requires 627.2, found: m/z=628.5 [M+H]⁺.

### Tert-butyl 4-[4-({4-[2-tert-butyl-4-(3-{[ethyl(methyl)sulfamoyl]amino}-2-fluorophenyl)-1,3-thiazol-5-yl]pyrimidin-2-yl}amino)-2-fluorophenyl]piperazine-1-carboxylate

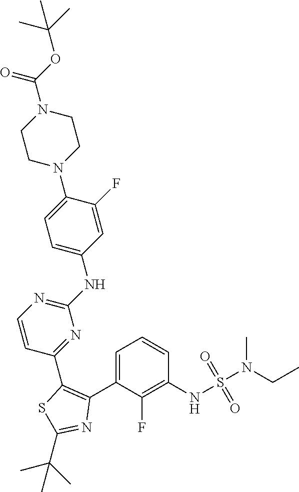 [SMILES: CCN(C)S(=O)(=O)Nc1cccc(-c2nc(C(C)(C)C)sc2-c2ccnc(Nc3ccc(N4CCN(C(=O)OC(C)(C)C)CC4)c(F)c3)n2)c1F]

LCMS: C₃₅H₄₄F₂N₈O₄S₂ requires 742.3, found: m/z=743.7 [M+H]⁺.

### 4-[2-tert-butyl-4-(3-{[ethyl(methyl)sulfamoyl]amino}-2-fluorophenyl)-1,3-thiazol-5-yl]-N-[3-fluoro-4-(piperazin-1-yl)phenyl]pyrimidin-2-amine

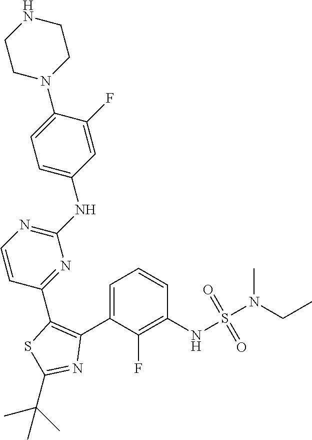 [SMILES: CCN(C)S(=O)(=O)Nc1cccc(-c2nc(C(C)(C)C)sc2-c2ccnc(Nc3ccc(N4CCNCC4)c(F)c3)n2)c1F]

LCMS: C₃₀H₃₆F₂N₈O₂S₂ requires 642.2, found: m/z=643.6 [M+H]⁺.

### Tert-butyl 4-(4-((4-(2-(tert-butyl)-4-(3-((N-ethyl-N-methylsulfamoyl)amino)-2-fluorophenyl)thiazol-5-yl)pyrimidin-2-yl)amino)-2-chlorophenyl)piperazine-1-carboxylate

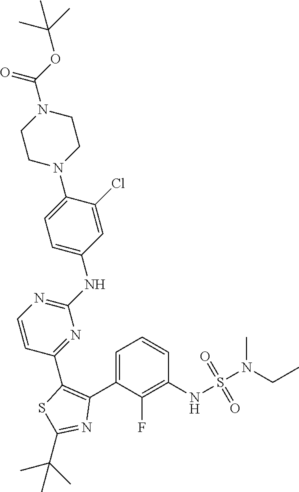 [SMILES: CCN(C)S(=O)(=O)Nc1cccc(-c2nc(C(C)(C)C)sc2-c2ccnc(Nc3ccc(N4CCN(C(=O)OC(C)(C)C)CC4)c(Cl)c3)n2)c1F]

LCMS: C₃₅H₄₄ClFN₈O₄S₂ requires 758.3, found: m/z=759.5 [M+H]⁺.

### 4-[2-tert-butyl-4-(3-{[ethyl(methyl)sulfamoyl]amino}-2-fluorophenyl)-1,3-thiazol-5-yl]-N-[3-chloro-4-(piperazin-1-yl)phenyl]pyrimidin-2-amine

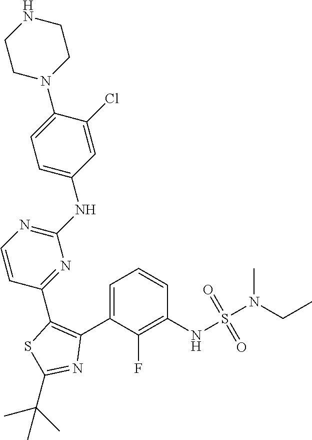 [SMILES: CCN(C)S(=O)(=O)Nc1cccc(-c2nc(C(C)(C)C)sc2-c2ccnc(Nc3ccc(N4CCNCC4)c(Cl)c3)n2)c1F]

LCMS: C₃₀H₃₆ClFN₈O₂S₂ requires 658.2, found: m/z=659.5 [M+H]⁺.

### General Procedure for SnAr Coupling Followed by Deprotection (Version 1)

### Synthesis of N-[3-(2-tert-butyl-5-{2-[(piperidin-4-ylmethyl)amino]pyrimidin-4-yl}-1,3-thiazol-4-yl)-2-fluorophenyl]propane-1-sulfonamide

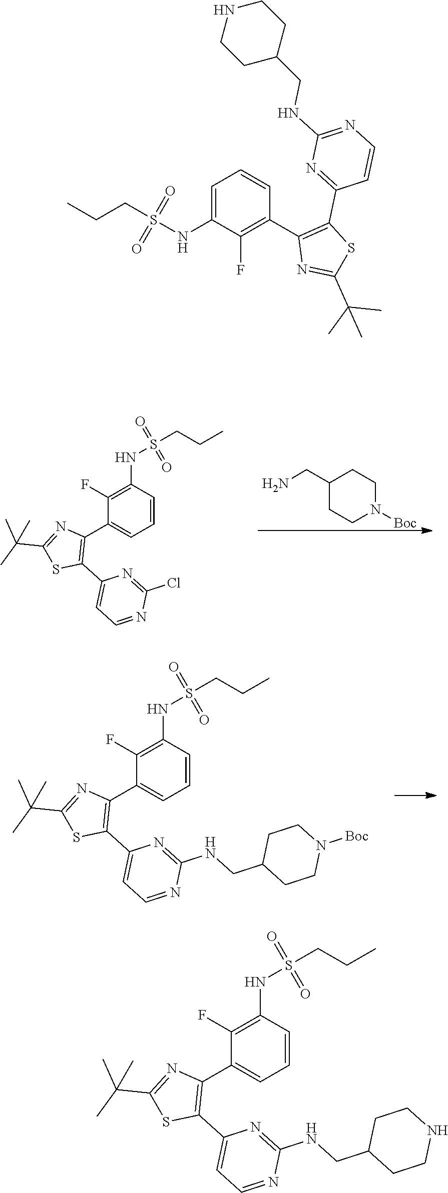

### Step-1: tert-butyl 4-(((4-(2-(tert-butyl)-4-(2-fluoro-3-(propylsulfonamido)phenyl)thiazol-5-yl)pyrimidin-2-yl)amino)methyl)piperidine-1-carboxylate

To a solution of N-{3-[2-tert-butyl-5-(2-chloropyrimidin-4-yl)-1,3-thiazol-4-yl]-2-fluorophenyl}propane-1-sulfonamide (850 mg, 1.81 mmol) and triethylamine (0.75 mL, 3 equiv) in dioxane (10 mL) and isopropanol (2.5 mL) was added tert-butyl 4-(aminomethyl)piperidine-1-carboxylate (505 mg, 1.3 equiv), and the solution was heated at 60° C. for 30 h. The reaction mixture was diluted with EtOAc (40 mL), and the resulting organic solution was washed with aqueous ammonium chloride, then brine, and dried with mag sulfate. Filtration and concentration of the organic solution provided the boc-protected intermediate tert-butyl 4-(((4-(2-(tert-butyl)-4-(2-fluoro-3-(propylsulfonamido)phenyl)thiazol-5-yl)pyrimidin-2-yl)amino)methyl)piperidine-1-carboxylate.

### Step-2: N-[3-(2-tert-butyl-5-{2-[(piperidin-4-ylmethyl)amino]pyrimidin-4-yl}-1,3-thiazol-4-yl)-2-fluorophenyl]propane-1-sulfonamide

The crude tert-butyl 4-(((4-(2-(tert-butyl)-4-(2-fluoro-3-(propylsulfonamido)phenyl)thiazol-5-yl)pyrimidin-2-yl)amino)methyl)piperidine-1-carboxylate was dissolved in DCM (10 mL) and TFA (3 mL). This reaction was stirred for 6 h, and was then concentrated onto silica. Silica gel column chromatography purification eluting with methanol:DCM (0-20%) provided N-[3-(2-tert-butyl-5-{2-[(piperidin-4-ylmethyl)amino]pyrimidin-4-yl}-1,3-thiazol-4-yl)-2-fluorophenyl]propane-1-sulfonamide (0.86 g, 87% over two steps). LCMS: C₂₆H₃₅FN₆O₂S₂ requires 546.2, found: m/z=547.5 [M+H]⁺.

The following compounds were made using the above General Procedure for SnAr Coupling Followed by Deprotection (Version 1).

### N-(3-{2-tert-butyl-5-[2-(pyrrolidin-3-ylamino)pyrimidin-4-yl]-1,3-thiazol-4-yl}-2-fluorophenyl)propane-1-sulfonamide

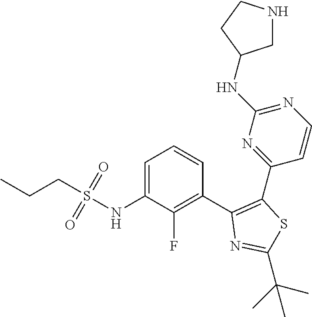 [SMILES: CCCS(=O)(=O)Nc1cccc(-c2nc(C(C)(C)C)sc2-c2ccnc(NC3CCNC3)n2)c1F]

LCMS: C₂₄H₃₁FN₆O₂S₂ requires 518.2, found: m/z=519.5 [M+H]⁺.

### [(4-{2-tert-butyl-4-[2-fluoro-3-(propane-1-sulfonamido)phenyl]-1,3-thiazol-5-yl}pyrimidin-2-yl)amino]acetic Acid

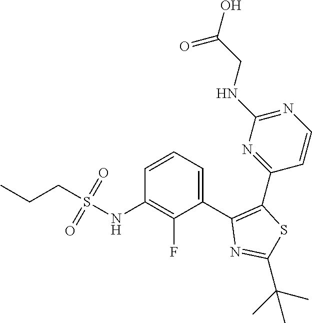 [SMILES: CCCS(=O)(=O)Nc1cccc(-c2nc(C(C)(C)C)sc2-c2ccnc(NCC(=O)O)n2)c1F]

LCMS: C₂₂H₂₆FN₅O₄S₂ requires 507.1, found: m/z=506.5 [M−H]⁺.

### N-(3-{2-tert-butyl-5-[2-(piperidin-4-ylamino)pyrimidin-4-yl]-1,3-thiazol-4-yl}-2-fluorophenyl)propane-1-sulfonamide

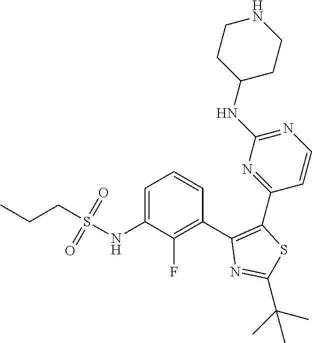 [SMILES: CCCS(=O)(=O)Nc1cccc(-c2nc(C(C)(C)C)sc2-c2ccnc(NC3CCNCC3)n2)c1F]

LCMS: C₂₅H₃₃FN₆O₂S₂ requires 532.2, found: m/z=533.5 [M+H]⁺.

### N-{3-[2-tert-butyl-5-(2-{[2-methyl-2-(piperidin-4-yl)propyl]amino}pyrimidin-4-yl)-1,3-thiazol-4-yl]-2-fluorophenyl}propane-1-sulfonamide

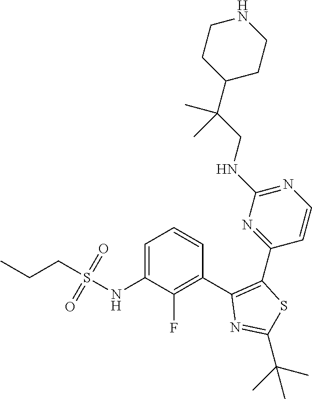 [SMILES: CCCS(=O)(=O)Nc1cccc(-c2nc(C(C)(C)C)sc2-c2ccnc(NCC(C)(C)C3CCNCC3)n2)c1F]

LCMS: C₂₉H₄₁FN₆O₂S₂ requires 588.3, found: m/z=589.7 [M−H]⁺.

### N-{3-[5-(2-{[1,4-bipiperidin]-4-ylamino}pyrimidin-4-yl)-2-tert-butyl-1,3-thiazol-4-yl]-2-fluorophenyl}propane-1-sulfonamide

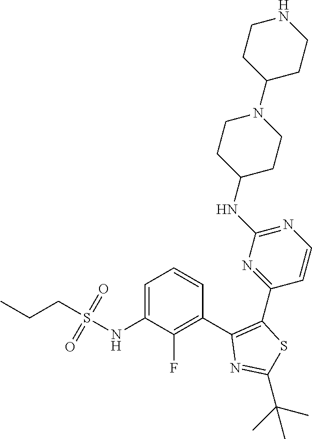 [SMILES: CCCS(=O)(=O)Nc1cccc(-c2nc(C(C)(C)C)sc2-c2ccnc(NC3CCN(C4CCNCC4)CC3)n2)c1F]

LCMS: C₃₀H₄₂FN₇O₂S₂ requires 615.3, found: m/z=616.6 [M+H]⁺.

### N-{3-[2-tert-butyl-5-(2-{[4-(piperazin-1-yl)cyclohexyl]amino}pyrimidin-4-yl)-1,3-thiazol-4-yl]-2-fluorophenyl}propane-1-sulfonamide

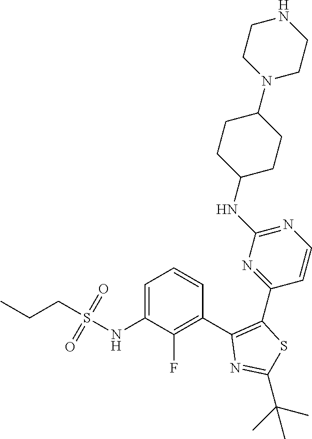 [SMILES: CCCS(=O)(=O)Nc1cccc(-c2nc(C(C)(C)C)sc2-c2ccnc(NC3CCC(N4CCNCC4)CC3)n2)c1F]

LCMS: C₃₀H₄₂FN₇O₂S₂ requires 615.3, found: m/z=616.6 [M+H]⁺.

### N-{3-[5-(2-{7-azaspiro[3.5]nonan-2-ylamino}pyrimidin-4-yl)-2-tert-butyl-1,3-thiazol-4-yl]-2-fluorophenyl}propane-1-sulfonamide

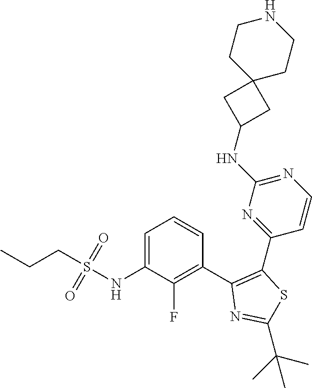 [SMILES: CCCS(=O)(=O)Nc1cccc(-c2nc(C(C)(C)C)sc2-c2ccnc(NC3CC4(CCNCC4)C3)n2)c1F]

LCMS: C₂₈H₃₇FN₆O₂S₂ requires 572.2, found: m/z=573.5 [M+H]⁺.

### General Procedure for Nucleophilic Substitution Followed by Oxidation

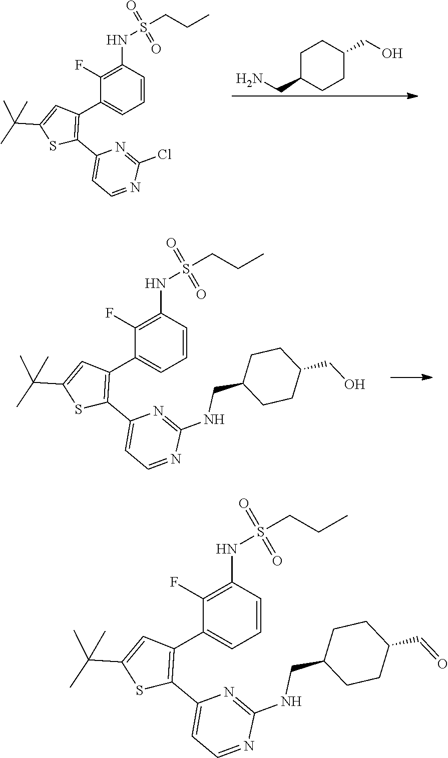 [SMILES: CCCS(=O)(=O)Nc1cccc(-c2cc(C(C)(C)C)sc2-c2ccnc(Cl)n2)c1F ; NC[C@H]1CC[C@H](CO)CC1 ; CCCS(=O)(=O)Nc1cccc(-c2cc(C(C)(C)C)sc2-c2ccnc(NC[C@H]3CC[C@H](CO)CC3)n2)c1F ; CCCS(=O)(=O)Nc1cccc(-c2cc(C(C)(C)C)sc2-c2ccnc(NC[C@H]3CC[C@H](C=O)CC3)n2)c1F]

### Step-1: N-(3-{2-tert-butyl-5-[2-({[(1r,4r)-4-(hydroxymethyl)cyclohexyl]methyl}amino)pyrimidin-4-yl]-1,3-thiazol-4-yl}-2-fluorophenyl)propane-1-sulfonamide

To a solution of N-{3-[2-tert-butyl-5-(2-chloropyrimidin-4-yl)-1,3-thiazol-4-yl]-2-fluorophenyl}propane-1-sulfonamide (950 mg, 2.03 mmol) and triethylamine (0.83 mL, 3 equiv) in dioxane (15 mL) and isopropanol (5 mL) was added [(1r,4r)-4-(aminomethyl)cyclohexyl]methanol (571 mg, 1.1 equiv) and the solution was heated at 60° C. for 18 h. The reaction mixture was diluted with EtOAc (40 mL) and the combined organic solution was washed with aqueous ammonium chloride, brine, dried over magnesium sulfate, filtered, and concentrated to provide N-(3-{2-tert-butyl-5-[2-({[(1r,4r)-4-(hydroxymethyl)cyclohexyl]methyl}amino)pyrimidin-4-yl]-1,3-thiazol-4-yl}-2-fluorophenyl)propane-1-sulfonamide (0.8 g). LCMS: C₂₈H₃₇FN₆O₂S₂ requires 575.2, found: m/z=576.7 [M+H]⁺.

### Step-2: N-(3-{2-tert-butyl-5-[2-({[(1r,4r)-4-formylcyclohexyl]methyl}amino)pyrimidin-4-yl]-1,3-thiazol-4-yl}-2-fluorophenyl)propane-1-sulfonamide

Crude N-(3-{2-tert-butyl-5-[2-({[(1r,4r)-4-(hydroxymethyl)cyclohexyl]methyl}amino)pyrimidin-4-yl]-1,3-thiazol-4-yl}-2-fluorophenyl)propane-1-sulfonamide from the previous reaction was dissolved in DCM at rt and then DMP (0.66 g, 1.1 equiv) was added in one portion. The reaction stirred for 2 h and then concentrated onto silica and purified by silica gel column chromatography (0-100% ethyl acetate in DCM) to provide N-(3-{2-tert-butyl-5-[2-({[(1r,4r)-4-formylcyclohexyl]methyl}amino)pyrimidin-4-yl]-1,3-thiazol-4-yl}-2-fluorophenyl)propane-1-sulfonamide (0.8 g, 70% over two steps). LCMS: C₂₈H₃₇FN₆O₂S₂ requires 573.2, found: m/z=574.7 [M+H]⁺.

The following compounds were made using the above General Procedure for Nucleophilic Substitution Followed by Oxidation.

### N-{3-[2-tert-butyl-5-(2-{[(1r,4r)-4-(hydroxymethyl)cyclohexyl]amino}pyrimidin-4-yl)-1,3-thiazol-4-yl]-2-fluorophenyl}propane-1-sulfonamide

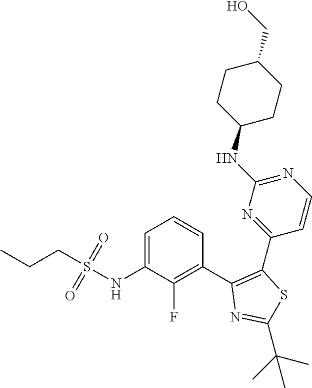 [SMILES: CCCS(=O)(=O)Nc1cccc(-c2nc(C(C)(C)C)sc2-c2ccnc(N[C@H]3CC[C@H](CO)CC3)n2)c1F]

LCMS: C₂₇H₃₆FN₅O₃S₂ requires 561.2, found: m/z=562.6 [M+H]⁺.

### N-(3-(2-(tert-butyl)-5-(2-(((1r,4r)-4-formylcyclohexyl)amino)pyrimidin-4-yl)thiazol-4-yl)-2-fluorophenyl)propane-1-sulfonamide

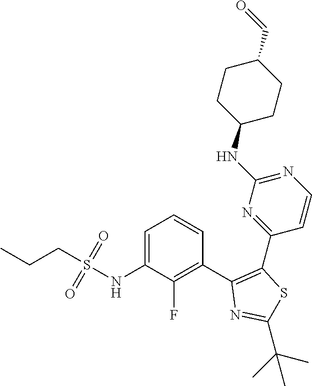 [SMILES: CCCS(=O)(=O)Nc1cccc(-c2nc(C(C)(C)C)sc2-c2ccnc(N[C@H]3CC[C@H](C=O)CC3)n2)c1F]

LCMS: C₂₇H₃₄FN₅O₃S₂ requires 559.2, found: m/z=560.5 [M+H]⁺.

### General Procedure for Amide Coupling Followed by Deprotection

### Synthesis of N-{3-[2-tert-butyl-5-(2-{[4-(piperazine-1-carbonyl)phenyl]amino}pyrimidin-4-yl)-1,3-thiazol-4-yl]-2-fluorophenyl}propane-1-sulfonamide

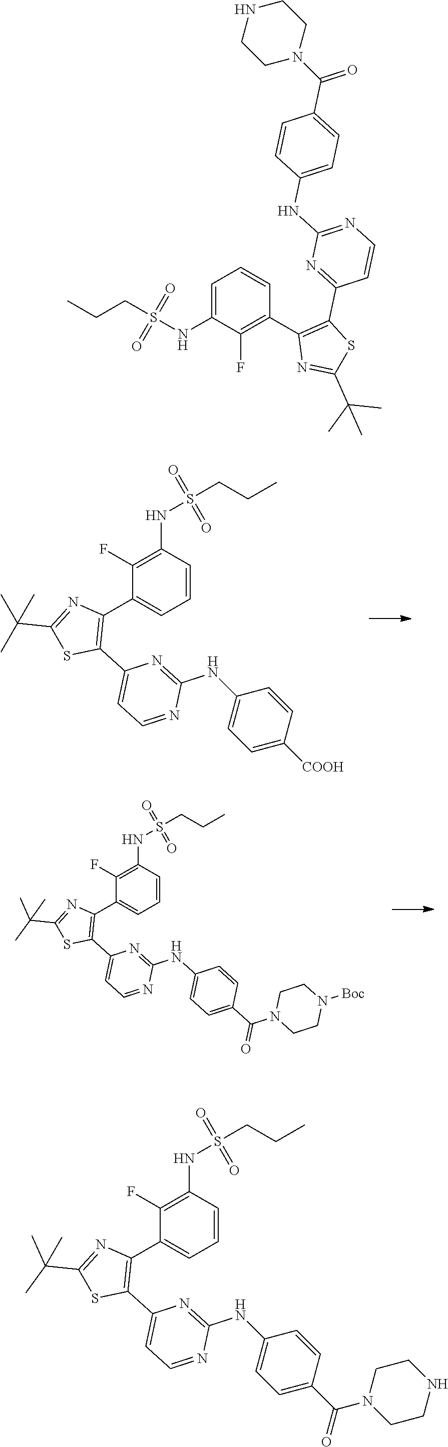

### Step-1: tert-butyl 4-(4-((4-(2-(tert-butyl)-4-(2-fluoro-3-(propylsulfonamido)phenyl)thiazol-5-yl)pyrimidin-2-yl)amino)benzoyl)piperazine-1-carboxylate

4-((4-(2-(tert-butyl)-4-(2-fluoro-3-(propylsulfonamido)phenyl)thiazol-5-yl)pyrimidin-2-yl)amino)benzoic acid (0.5 g, 0.887 mmol), and HATU (0.33 g, 1 equiv) were dissolved in DMF (0.2 M) and DIEA (0.1 mL) followed by the addition of tert-butyl piperazine-1-carboxylate (0.16 g, 1 equiv). The reaction was stirred for 2 h, followed by partition between water and ethyl acetate. The organic layer was separated, washed with brine, dried over magnesium sulfate, filtered, and concentrated. Crude tert-butyl 4-(4-((4-(2-(tert-butyl)-4-(2-fluoro-3-(propylsulfonamido)phenyl)thiazol-5-yl)pyrimidin-2-yl)amino)benzoyl)piperazine-1-carboxylate was used in the next step without purification.

### Step-2: N-{3-[2-tert-butyl-5-(2-{[4-(piperazine-1-carbonyl)phenyl]amino}pyrimidin-4-yl)-1,3-thiazol-4-yl]-2-fluorophenyl}propane-1-sulfonamide

Crude tert-butyl 4-(4-((4-(2-(tert-butyl)-4-(2-fluoro-3-(propylsulfonamido)phenyl)thiazol-5-yl)pyrimidin-2-yl)amino)benzoyl)piperazine-1-carboxylate was dissolved in DCM (10 mL) and TFA (2 mL) was added. The reaction was then stirred for 3 h and then concentrated onto silica gel. Silica gel column chromatography (0-20% methanol in DCM) provided N-{3-[2-tert-butyl-5-(2-{[4-(piperazine-1-carbonyl)phenyl]amino}pyrimidin-4-yl)-1,3-thiazol-4-yl]-2-fluorophenyl}propane-1-sulfonamide (0.2 g, 36%). LCMS: C₃₁H₃₆FN₇O₃S₂ requires 637.2, found: m/z=638.5 [M+H]⁺.

### General Procedure for Buchwald Coupling Followed by BOC Deprotection

### Synthesis of N-(3-(2-(tert-butyl)-5-(2-((4-(piperidin-4-yl)phenyl)amino)pyrimidin-4-yl)thiazol-4-yl)-2-fluorophenyl)propane-1-sulfonamide

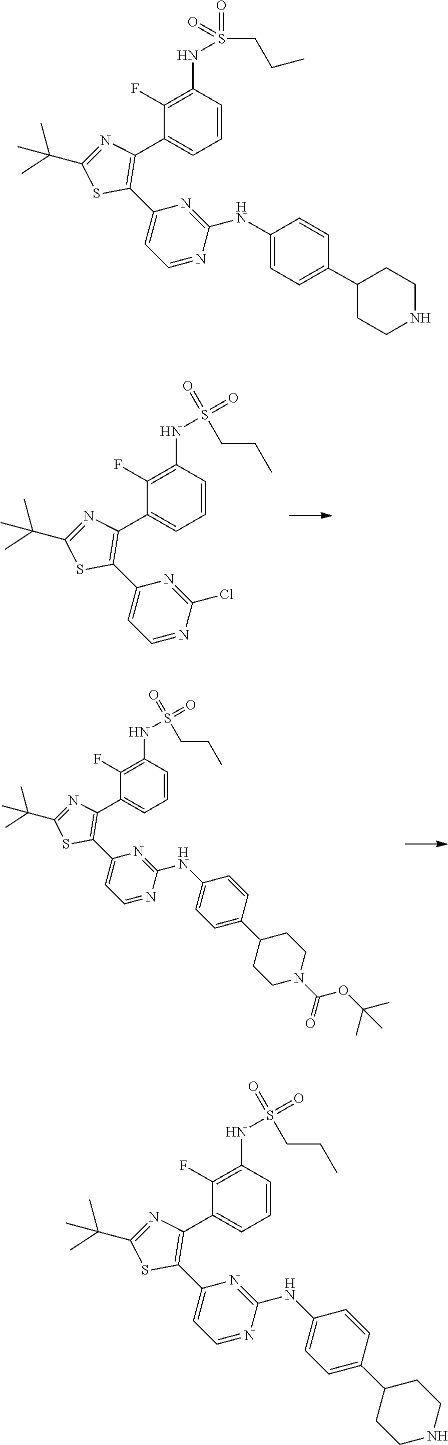

### Step-1: tert-butyl 4-(4-((4-(2-(tert-butyl)-4-(2-fluoro-3-(propylsulfonamido)phenyl)thiazol-5-yl)pyrimidin-2-yl)amino)phenyl)piperidine-1-carboxylate

To a solution of N-{3-[2-tert-butyl-5-(2-chloropyrimidin-4-yl)-1,3-thiazol-4-yl]-2-fluorophenyl}propane-1-sulfonamide (600.00 mg, 1.2793 mmol) and tert-butyl 4-(4-aminophenyl)piperidine-1-carboxylate (530.38 mg, 1.9190 mmol) in dioxane (15.00 mL) was added sodium tert-butoxide (184.43 mg, 1.9190 mmol), Pd₂(dba)₃ (117.15 mg, 0.1279 mmol), and Dave-Phos 2′-(dicyclohexylphosphanyl)-N,N-dimethyl-[1,1′-biphenyl]-2-amine (503.49 mg, 1.2793 mmol). The mixture was purged with nitrogen gas for five minutes. The reaction mixture was then heated by microwave at 120° C. for two hours. Solvent was evaporated under reduced pressure. The crude product was loaded onto a Redi-Sep prepacked silica gel column eluting with 0-70% EtOAc in hexanes, followed by a second purification using a Redi-Sep prepacked silica gel column purification eluting with a gradient of 0-40% EtOAc in DCM to afford tert-butyl 4-{4-[(4-{2-tert-butyl-4-[2-fluoro-3-(propane-1-sulfonamido)phenyl]-1,3-thiazol-5-yl}pyrimidin-2-yl)amino]phenyl}piperidine-1-carboxylate (0.605 g, 66.71%). LCMS: C₃₆H₄₅FN₆O₄S₂, requires: 708.29, found: m/z=709.51 [M+H]⁺. ¹H NMR (500 MHz, DMSO-d₆) δ 9.65 (s, 1H), 8.33 (d, J=5.2 Hz, 1H), 7.56 (dd, J=16.5, 8.0 Hz, 3H), 7.37 (t, J=7.9 Hz, 1H), 7.13 (d, J=8.4 Hz, 2H), 3.07-2.95 (m, 2H), 2.81 (s, 2H), 1.75 (d, J=12.8 Hz, 2H), 1.66 (p, J=7.5 Hz, 2H), 1.47 (s, 9H), 1.43 (s, 9H), 0.90 (t, J=7.4 Hz, 3H).

### Step-2: N-(3-(2-(tert-butyl)-5-(2-((4-(piperidin-4-yl)phenyl)amino)pyrimidin-4-yl)thiazol-4-yl)-2-fluorophenyl)propane-1-sulfonamide

To a solution of tert-butyl 4-{4-[(4-{2-tert-butyl-4-[2-fluoro-3-(propane-1-sulfonamido)phenyl]-1,3-thiazol-5-yl}pyrimidin-2-yl)amino]phenyl}piperidine-1-carboxylate (0.55 g, 0.903 mmol) in DCM (5 mL) was added 4N HCl in dioxane solution (5 mL). The solution was stirred for two hours. Solvent was removed under reduced pressure and the product was lyophilized to dryness to afford N-{3-[2-tert-butyl-5-(2-{[4-(piperidin-4-yl)phenyl]amino}pyrimidin-4-yl)-1,3-thiazol-4-yl]-2-fluorophenyl}propane-1-sulfonamide (0.55 g, 94.6%) as an HCl salt. LCMS: C₃₁H₃₇FN₆O₂S₂, requires: 608.24, found: m/z=609.47 [M+H]⁺.

The following compounds were made using the above General Procedure for Buchwald Coupling Followed by BOC Deprotection.
4-[(4-{2-tert-butyl-4-[2-fluoro-3-(propane-1-sulfonamido)phenyl]-1,3-thiazol-5-yl}pyrimidin-2-yl)amino]benzoic acid [M+H]⁺ 626.5, then 570.5.
N-{3-[2-tert-butyl-5-(2-{[3-fluoro-4-(piperazin-1-yl)phenyl]amino}pyrimidin-4-yl)-1,3-thiazol-4-yl]-2-fluorophenyl}propane-1-sulfonamide [M+H]⁺ 728.5, then 628.5.
4-[2-tert-butyl-4-(3-{[ethyl(methyl)sulfamoyl]amino}-2-fluorophenyl)-1,3-thiazol-5-yl]-N-[3-fluoro-4-(piperazin-1-yl)phenyl]pyrimidin-2-amine [M+H]⁺ 743.7, then 643.6.
4-[2-tert-butyl-4-(3-{[ethyl(methyl)sulfamoyl]amino}-2-fluorophenyl)-1,3-thiazol-5-yl]-N-[3-chloro-4-(piperazin-1-yl)phenyl]pyrimidin-2-amine [M+H]⁺ 759.5, then 659.5 (Boc deprotection).

The following scheme uses the above General Procedure for Buchwald Coupling Followed by BOC Deprotection.

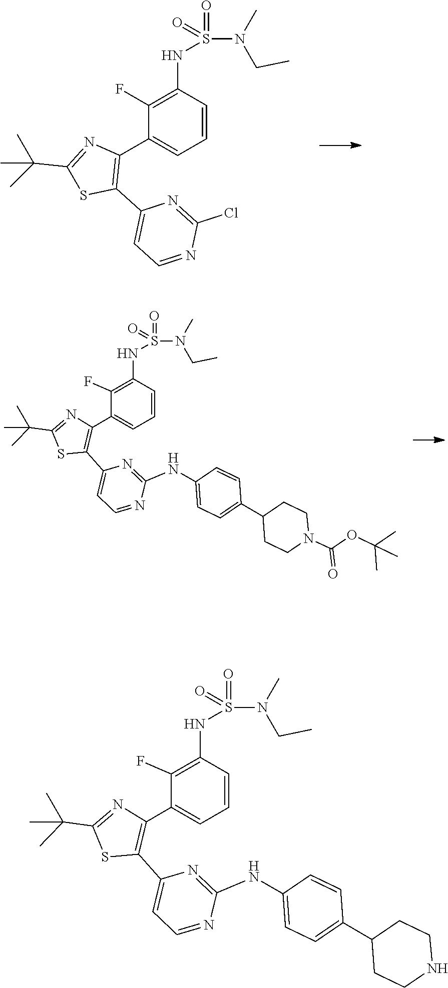

### Tert-butyl 4-[4-({4-[2-tert-butyl-4-(3-{[ethyl(methyl)sulfamoyl]amino}-2-fluorophenyl)-1,3-thiazol-5-yl]pyrimidin-2-yl}amino)phenyl]piperidine-1-carboxylate

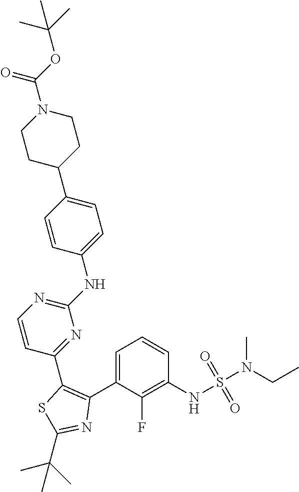 [SMILES: CCN(C)S(=O)(=O)Nc1cccc(-c2nc(C(C)(C)C)sc2-c2ccnc(Nc3ccc(C4CCN(C(=O)OC(C)(C)C)CC4)cc3)n2)c1F]

LCMS: C₃₆H₄₆FN₇O₄S₂, requires: 723.3, found: m/z=724.5 [M+H]⁺.

### 4-[2-tert-butyl-4-(3-f{[ethyl(methyl)sulfamoyl]amino}-2-fluorophenyl)-1,3-thiazol-5-yl]-N-[4-(piperidin-4-yl)phenyl]pyrimidin-2-amine

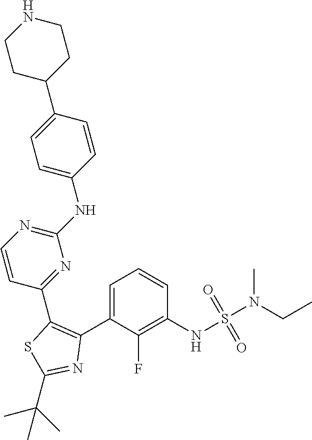 [SMILES: CCN(C)S(=O)(=O)Nc1cccc(-c2nc(C(C)(C)C)sc2-c2ccnc(Nc3ccc(C4CCNCC4)cc3)n2)c1F]

LCMS: C₃₁H₃₈FN₇O₂S₂, requires: 623.3, found: m/z=624.4 [M+H]⁺. ¹H NMR (500 MHz, DMSO-d₆) δ 9.70 (s, 2H), 8.82 (s, 1H), 7.64 (d, J=8.3 Hz, 2H), 7.56 (t, J=7.7 Hz, 1H), 7.40 (t, J=6.7 Hz, 1H), 7.35 (t, J=7.9 Hz, 1H), 7.13 (d, J=8.3 Hz, 2H), 3.75-3.65 (m, 6H), 3.49 (dd, J=15.3, 4.9 Hz, 1H), 3.37 (d, J=12.4 Hz, 2H), 3.08 (d, J=7.2 Hz, 1H), 3.05 (d, J=7.2 Hz, 1H), 2.99 (d, J=12.1 Hz, 2H), 2.84-2.74 (m, 1H), 2.67 (s, 2H), 1.93 (d, J=13.7 Hz, 2H), 1.81 (q, J=13.1 Hz, 2H), 1.47 (s, 9H), 1.00 (t, J=7.1 Hz, 3H).

The following scheme uses the above General Procedure for Buchwald Coupling Followed by BOC Deprotection.

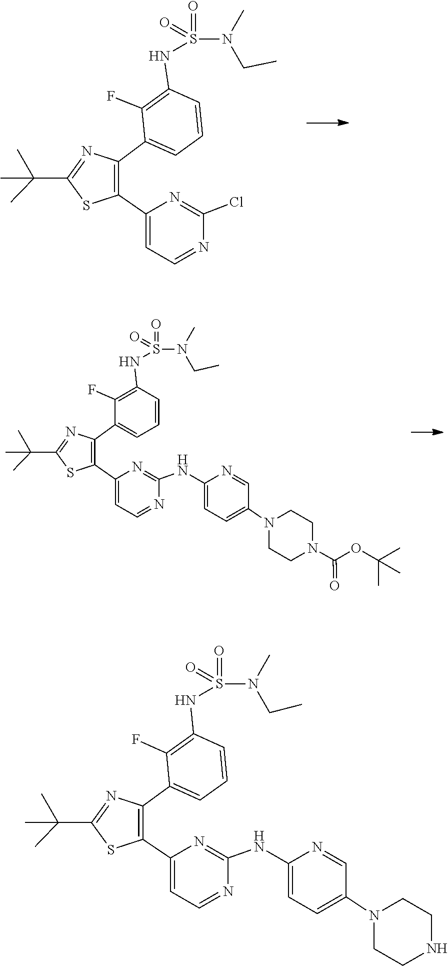

### Tert-butyl 4-[6-({4-[2-tert-butyl-4-(3-{[ethyl(methyl)sulfamoyl]amino}-2-fluorophenyl)-1,3-thiazol-5-yl]pyrimidin-2-yl}amino)pyridin-3-yl]piperazine-1-carboxylate

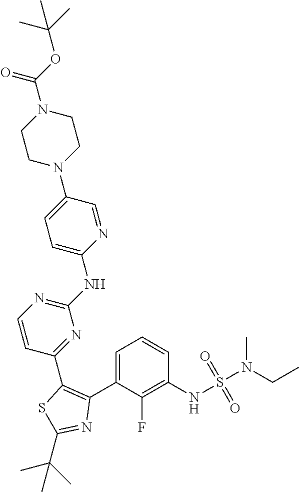 [SMILES: CCN(C)S(=O)(=O)Nc1cccc(-c2nc(C(C)(C)C)sc2-c2ccnc(Nc3ccc(N4CCN(C(=O)OC(C)(C)C)CC4)cn3)n2)c1F]

LCMS: C₃₄H₄₄FN₉O₄S₂ requires: 725.29, found: m/z=726.52 [M+H]⁺.

### 4-[6-({4-[2-tert-butyl-4-(3-{[ethyl(methyl)sulfamoyl]amino}-2-fluorophenyl)-1,3-thiazol-5-yl]pyrimidin-2-yl}amino)pyridin-3-yl]piperazine

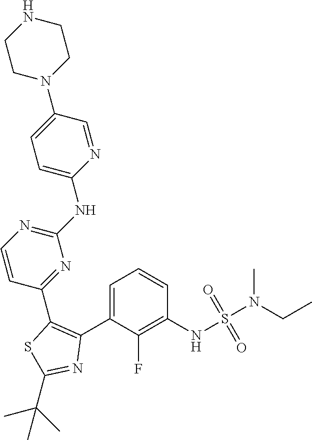 [SMILES: CCN(C)S(=O)(=O)Nc1cccc(-c2nc(C(C)(C)C)sc2-c2ccnc(Nc3ccc(N4CCNCC4)cn3)n2)c1F]

LCMS: C₂₉H₃₆FN₉O₂S₂ requires: 625.24, found: m/z=626.44 [M+H]⁺. ¹H NMR (500 MHz, DMSO-d₆) δ 10.92 (s, 1H), 9.68 (s, 1H), 9.17 (s, 2H), 8.50 (d, J=5.4 Hz, 1H), 8.06 (s, 1H), 7.92 (s, 1H), 7.88-7.84 (m, 1H), 7.74 (d, J=9.3 Hz, 1H), 7.54 (t, J=7.9 Hz, 1H), 7.45 (t, J=6.8 Hz, 1H), 7.36 (t, J=7.9 Hz, 1H), 6.68 (d, J=5.3 Hz, 1H), 5.16 (d, J=7.4 Hz, 1H), 3.75-3.65 (m, 1H), 3.54-3.44 (m, 1H), 3.40 (t, J=5.2 Hz, 4H), 3.07 (q, J=7.1 Hz, 2H), 2.68 (s, 3H), 1.48 (s, 9H), 1.01 (t, J=7.1 Hz, 3H).

The following scheme uses the above General Procedure for Buchwald Coupling Followed by BOC Deprotection.

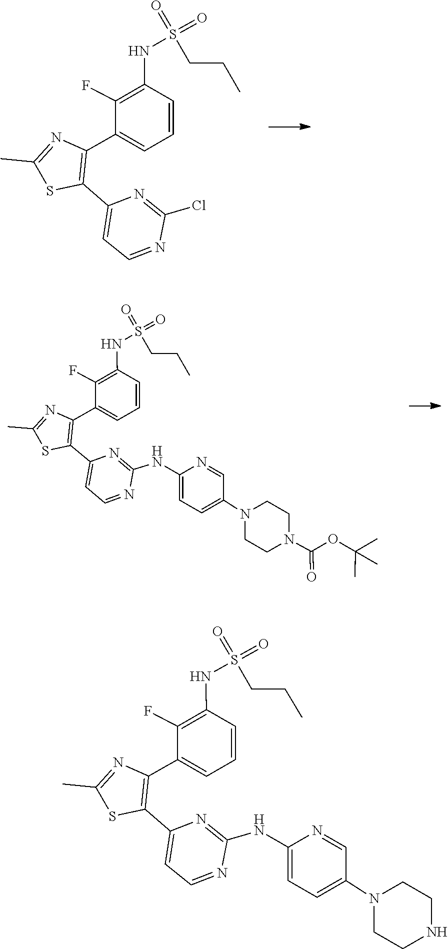

### Tert-butyl 4-{6-[(4-{4-[2-fluoro-3-(propane-1-sulfonamido)phenyl]-2-methyl-1,3-thiazol-5-yl}pyrimidin-2-yl)amino]pyridin-3-yl}piperazine-1-carboxylate

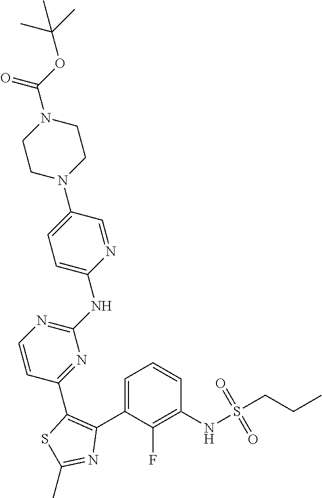 [SMILES: CCCS(=O)(=O)Nc1cccc(-c2nc(C)sc2-c2ccnc(Nc3ccc(N4CCN(C(=O)OC(C)(C)C)CC4)cn3)n2)c1F]

LCMS: C₃₁H₃₇FN₈O₄S₂ requires: 668.23, found: m/z=669.51 [M+H]⁺. ¹H NMR (500 MHz, CDCl₃) δ 8.31 (d, J=5.2 Hz, 1H), 8.29 (s, 1H), 8.08 (d, J=9.0 Hz, 1H), 8.03 (d, J=2.9 Hz, 1H), 7.75-7.67 (m, 1H), 7.41 (td, J=7.2, 6.5, 1.7 Hz, 1H), 7.35-7.29 (m, 2H), 6.83 (d, J=17.4 Hz, 1H), 6.50 (d, J=5.2 Hz, 1H), 3.63 (t, J=5.1 Hz, 4H), 3.12 (t, J=5.1 Hz, 4H), 3.04 (ddd, J=9.4, 5.6, 1.4 Hz, 2H), 2.82 (d, J=0.9 Hz, 3H), 1.84 (q, J=7.8 Hz, 2H), 1.52 (s, 9H), 1.01 (td, J=7.4, 1.4 Hz, 3H).

### N-{2-fluoro-3-[2-methyl-5-(2-{[5-(piperazin-1-yl)pyridin-2-yl]amino}pyrimidin-4-yl)-1,3-thiazol-4-yl]phenyl}propane-1-sulfonamide

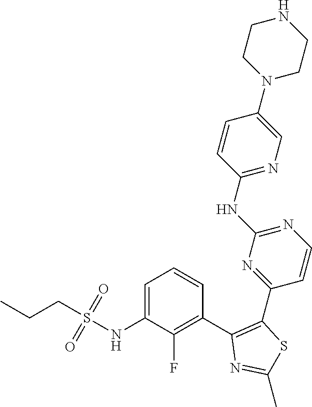 [SMILES: CCCS(=O)(=O)Nc1cccc(-c2nc(C)sc2-c2ccnc(Nc3ccc(N4CCNCC4)cn3)n2)c1F]

LCMS: C₂₆H₂₉FN₈O₂S₂ requires: 568.18, found: m/z=569.51 [M+H]⁺.

### General Procedure for SnAr Coupling Followed by Deprotection (Version 2)

### Synthesis of N-(3-(5-(2-((2-azaspiro[3.3]heptan-6-yl)amino)pyrimidin-4-yl)-2-(tert-butyl)thiazol-4-yl)-2-fluorophenyl)propane-1-sulfonamide

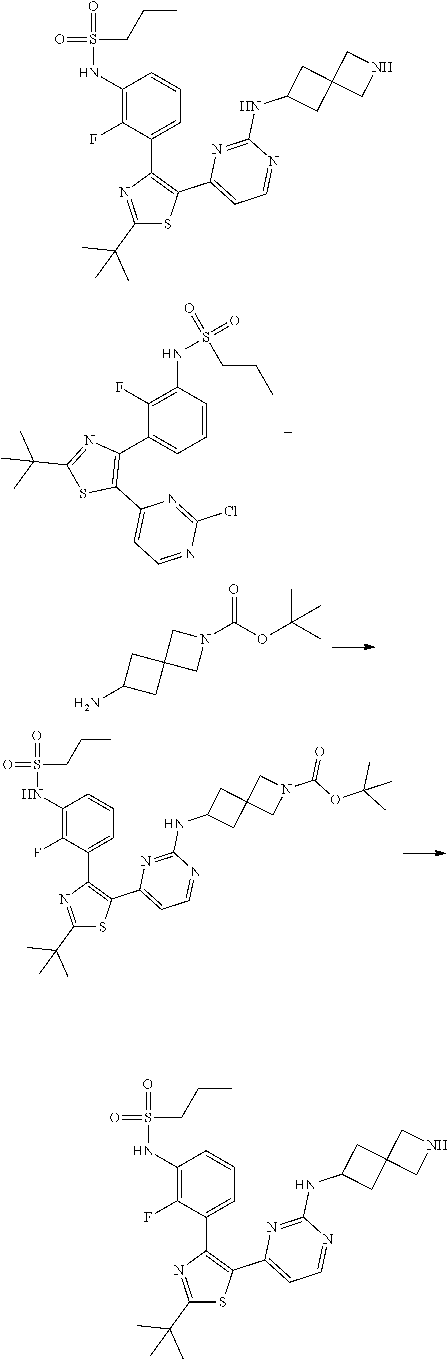

### Step-1: tert-butyl 6-((4-(2-(tert-butyl)-4-(2-fluoro-3-(propylsulfonamido)phenyl)thiazol-5-yl)pyrimidin-2-yl)amino)-2-azaspiro[3.3]heptane-2-carboxylate

To a solution of N-{3-[2-tert-butyl-5-(2-chloropyrimidin-4-yl)-1,3-thiazol-4-yl]-2-fluorophenyl}propane-1-sulfonamide (800.00 mg, 1.7058 mmol) and triethylamine (0.70 mL, 5.1174 mmol) in dioxane (2.50 mL) and isopropanol (2.50 mL) was added tert-butyl 6-amino-2-azaspiro[3.3]heptane-2-carboxylate (543.19 mg, 2.5587 mmol). The reaction mixture was heated at 60° C. for sixty hours. The reaction mixture was then cooled down and diluted with EtOAc (40 mL). The organic solution was washed by 20% citric acid solution, dried over sodium sulfate, filtered, and concentrated under reduced pressure. The crude product was loaded onto a Redi-Sep prepacked silica gel column eluting with a gradient of 15-90% EtOAc in hexanes, to provide tert-butyl 6-[(4-{2-tert-butyl-4-[2-fluoro-3-(propane-1-sulfonamido)phenyl]-1,3-thiazol-5-yl}pyrimidin-2-yl)amino]-2-azaspiro[3.3]heptane-2-carboxylate (0.86 g, 78.19%). LCMS: C₃₁H₄₁FN₆O₄S₂ required: 644.26 found: m/z=645.49 [M+H]⁺.

### Step-2: N-(3-(5-(2-((2-azaspiro[3.3]heptan-6-yl)amino)pyrimidin-4-yl)-2-(tert-butyl)thiazol-4-yl)-2-fluorophenyl)propane-1-sulfonamide

To a solution of tert-butyl 6-[(4-{2-tert-butyl-4-[2-fluoro-3-(propane-1-sulfonamido)phenyl]-1,3-thiazol-5-yl}pyrimidin-2-yl)amino]-2-azaspiro[3.3]heptane-2-carboxylate (860.00 mg, 1.3337 mmol) in hexafluoro-2-propanol (4 mL) was added TFA (0.5 mL) and the reaction was stirred for one hour at room temperature. The solution was concentrated in vacuo. The dried product was dissolved in DCM (30 mL), washed with sat. NaHCO₃ solution, and concentrated in vacuo. The product was lyophilized to provide N-{3-[5-(2-{2-azaspiro[3.3]heptan-6-ylamino}pyrimidin-4-yl)-2-tert-butyl-1,3-thiazol-4-yl]-2-fluorophenyl}propane-1-sulfonamide (0.661 g, 90.99%) as a yellow oil. LCMS: C₂₆H₃₃FN₆O₂S₂, requires: 544.21, found: m/z=545.34 [M+H]⁺.

The following scheme uses the above General Procedure for SnAr Coupling Followed by Deprotection (Version 2).

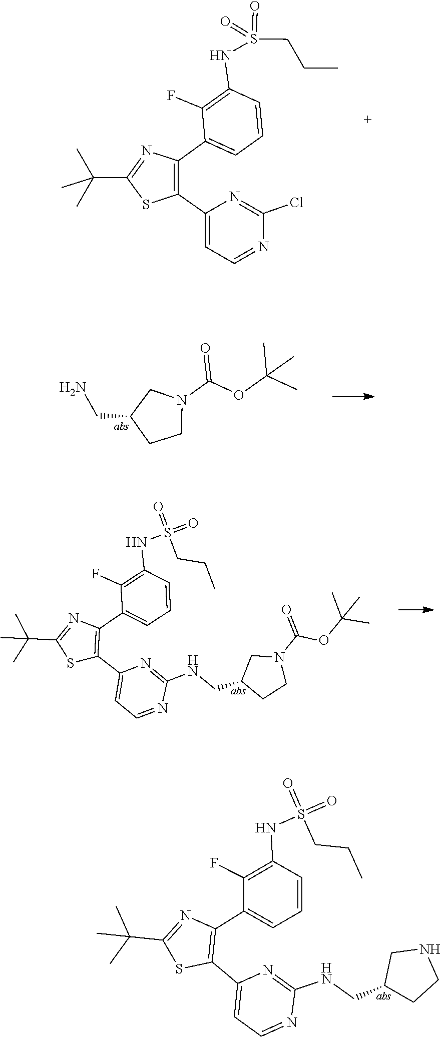

### Tert-butyl (3R)-3-{[(4-{2-tert-butyl-4-[2-fluoro-3-(propane-1-sulfonamido)phenyl]-1,3-thiazol-5-yl}pyrimidin-2-yl)amino]methyl}pyrrolidine-1-carboxylate

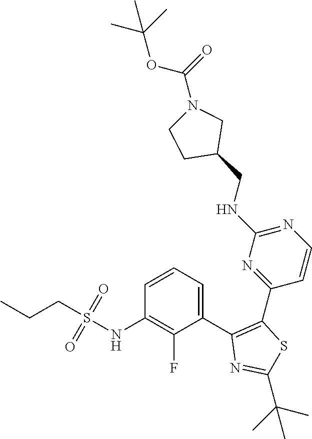 [SMILES: CCCS(=O)(=O)Nc1cccc(-c2nc(C(C)(C)C)sc2-c2ccnc(NC[C@H]3CCN(C(=O)OC(C)(C)C)C3)n2)c1F]

LCMS: C₃₀H₄₁FN₆O₄S₂ requires: 632.26, found: m/z=633.30 [M+H]⁺.

### N-{3-[2-tert-butyl-5-(2-{[(3S)-pyrrolidin-3-ylmethyl]amino}pyrimidin-4-yl)-1,3-thiazol-4-yl]-2-fluorophenyl}propane-1-sulfonamide

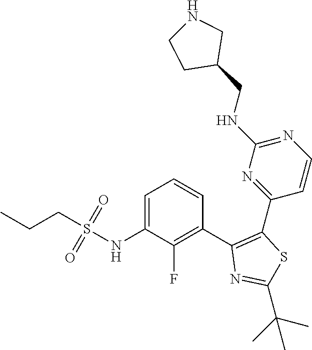 [SMILES: CCCS(=O)(=O)Nc1cccc(-c2nc(C(C)(C)C)sc2-c2ccnc(NC[C@H]3CCNC3)n2)c1F]

LCMS: C₂₅H₃₃FN₆O₂S₂, requires: 532.21, found: m/z=533.69 [M+H]⁺.

The following scheme uses the above General Procedure for SnAr Coupling Followed by Deprotection (Version 2).

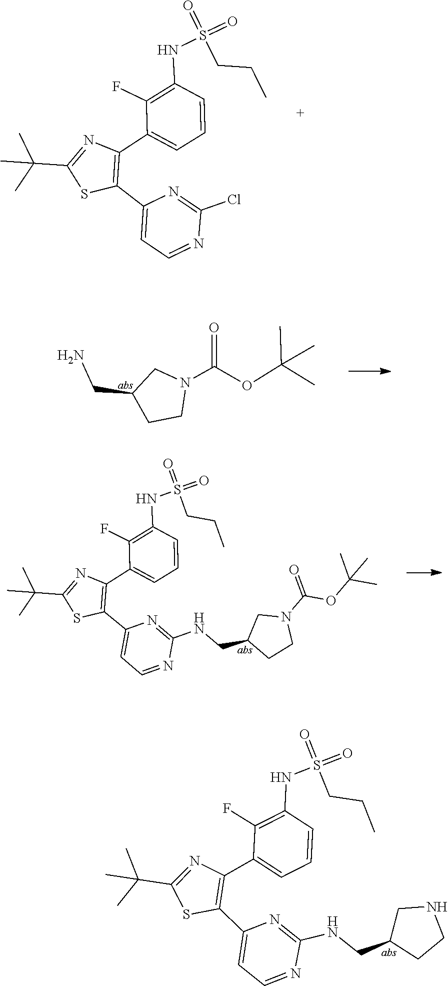

### Tert-butyl (3S)-3-{[(4-{2-tert-butyl-4-[2-fluoro-3-(propane-1-sulfonamido)phenyl]-1,3-thiazol-5-yl}pyrimidin-2-yl)amino]methyl}pyrrolidine-1-carboxylate

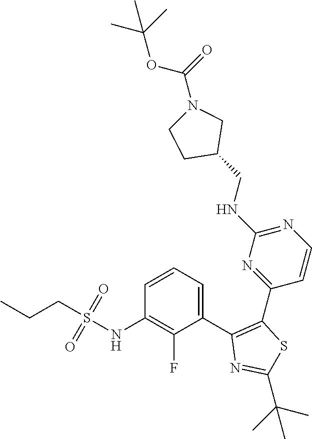 [SMILES: CCCS(=O)(=O)Nc1cccc(-c2nc(C(C)(C)C)sc2-c2ccnc(NC[C@@H]3CCN(C(=O)OC(C)(C)C)C3)n2)c1F]

LCMS: C₃₀H₄₁FN₆O₄S₂ requires: 632.26, found: m/z=633.86 [M+H]⁺.

### N-{3-[2-tert-butyl-5-(2-{[(3R)-pyrrolidin-3-ylmethyl]amino}pyrimidin-4-yl)-1,3-thiazol-4-yl]-2-fluorophenyl}propane-1-sulfonamide

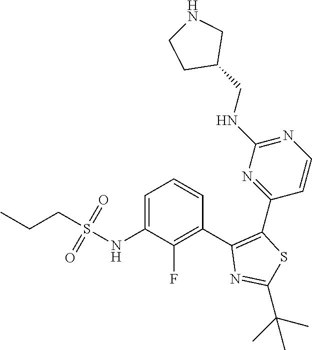 [SMILES: CCCS(=O)(=O)Nc1cccc(-c2nc(C(C)(C)C)sc2-c2ccnc(NC[C@@H]3CCNC3)n2)c1F]

LCMS: C₂₅H₃₃FN₆O₂S₂, requires: 532.21, found: m/z=533.76 [M+H]⁺.

The following scheme uses a modified General Procedure for Amide Coupling Followed by Deprotection.

### Synthesis of N-(3-(2-(tert-butyl)-5-(2-((2-(4-fluoropiperidine-4-carbonyl)-2-azaspiro[3.3]heptan-6-yl)amino)pyrimidin-4-yl)thiazol-4-yl)-2-fluorophenyl)propane-1-sulfonamide

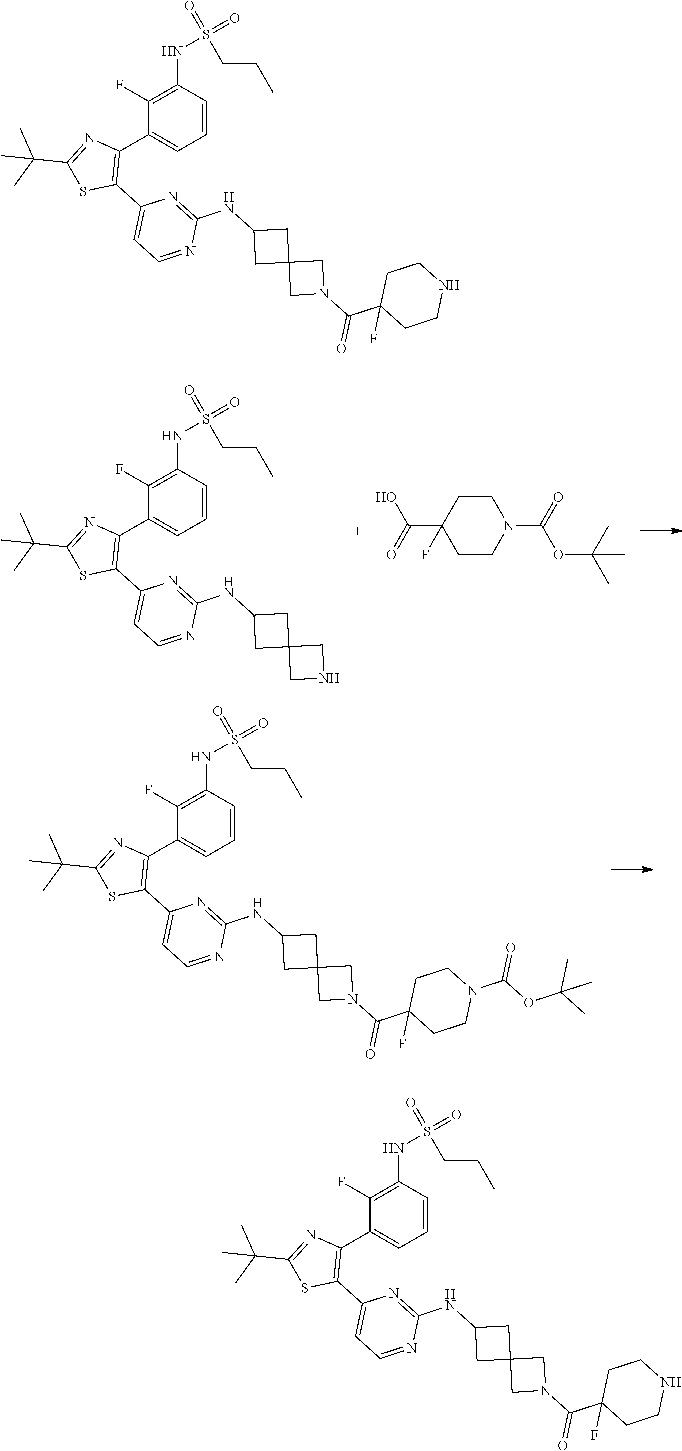

### Step-1: tert-butyl 4-(6-((4-(2-(tert-butyl)-4-(2-fluoro-3-(propylsulfonamido)phenyl)thiazol-5-yl)pyrimidin-2-yl)amino)-2-azaspiro[3.3]heptane-2-carbonyl)-4-fluoropiperidine-1-carboxylate

To a solution of 1-(tert-butoxycarbonyl)-4-fluoropiperidine-4-carboxylic acid (23.15 mg, 0.0936 mmol) and [(dimethylamino)({[1,2,3]triazolo[4,5-b]pyridine-3-yloxy})methylidene]dimethylazanium; hexafluoro-lambda5-phosphanide (35.60 mg, 0.0936 mmol) in DMF (0.4 mL) was added N,N-diisopropylethylamine (40.34 mg, 0.3121 mmol) followed by the addition of N-{3-[5-(2-{2-azaspiro[3.3]heptan-6-ylamino}pyrimidin-4-yl)-2-tert-butyl-1,3-thiazol-4-yl]-2-fluorophenyl}propane-1-sulfonamide (34.00 mg, 0.0624 mmol) in DMF (0.2 mL). The reaction was stirred at room temperature for twenty minutes. The reaction mixture was then diluted with EtOAc (20 mL). The organic solution was washed with water (0.5 mL) twice, dried over sodium sulfate, filtered, and concentrated in vacuo. The product was loaded onto a Redi-Sep prepacked silica gel column eluting with a gradient of 0-10% DCM in MeOH to afford tert-butyl 4-{6-[(4-{2-tert-butyl-4-[2-fluoro-3-(propane-1-sulfonamido)phenyl]-1,3-thiazol-5-yl}pyrimidin-2-yl)amino]-2-azaspiro[3.3]heptane-2-carbonyl}-4-fluoropiperidine-1-carboxylate (0.046 g, 95.22%). LCMS: C₃₇H₄₉F₂N₇O₅S₂ requires: 773.32, found: m/z=775.04.

### Step-2: N-(3-(2-(tert-butyl)-5-(2-((2-(4-fluoropiperidine-4-carbonyl)-2-azaspiro[3.3]heptan-6-yl)amino)pyrimidin-4-yl)thiazol-4-yl)-2-fluorophenyl)propane-1-sulfonamide

Tert-butyl 4-{6-[(4-{2-tert-butyl-4-[2-fluoro-3-(propane-1-sulfonamido)phenyl]-1,3-thiazol-5-yl}pyrimidin-2-yl)amino]-2-azaspiro[3.3]heptane-2-carbonyl}-4-fluoropiperidine-1-carboxylate (46.00 mg, 0.0594 mmol) was dissolved in DCM (2 mL) and 4N HCl in dioxane (0.30 mL, 1.1887 mmol) was added. The reaction was stirred for one hour and the solvent was removed in vacuo. N-(3-(2-(tert-butyl)-5-(2-((2-(4-fluoropiperidine-4-carbonyl)-2-azaspiro[3.3]heptan-6-yl)amino)pyrimidin-4-yl)thiazol-4-yl)-2-fluorophenyl)propane-1-sulfonamide was isolated without further purification. LCMS: C₃₂H₄₁F₂N₇O₃S₂ requires: 673.27, found: m/z=674.84 [M+H]⁺.

### General Procedure for Reductive Alkylation Followed by Deprotection

### Synthesis of N-(3-(5-(2-aminopyrimidin-4-yl)-2-(1-(azetidin-3-yl)piperidin-4-yl)thiazol-4-yl)-2-fluorophenyl)propane-1-sulfonamide

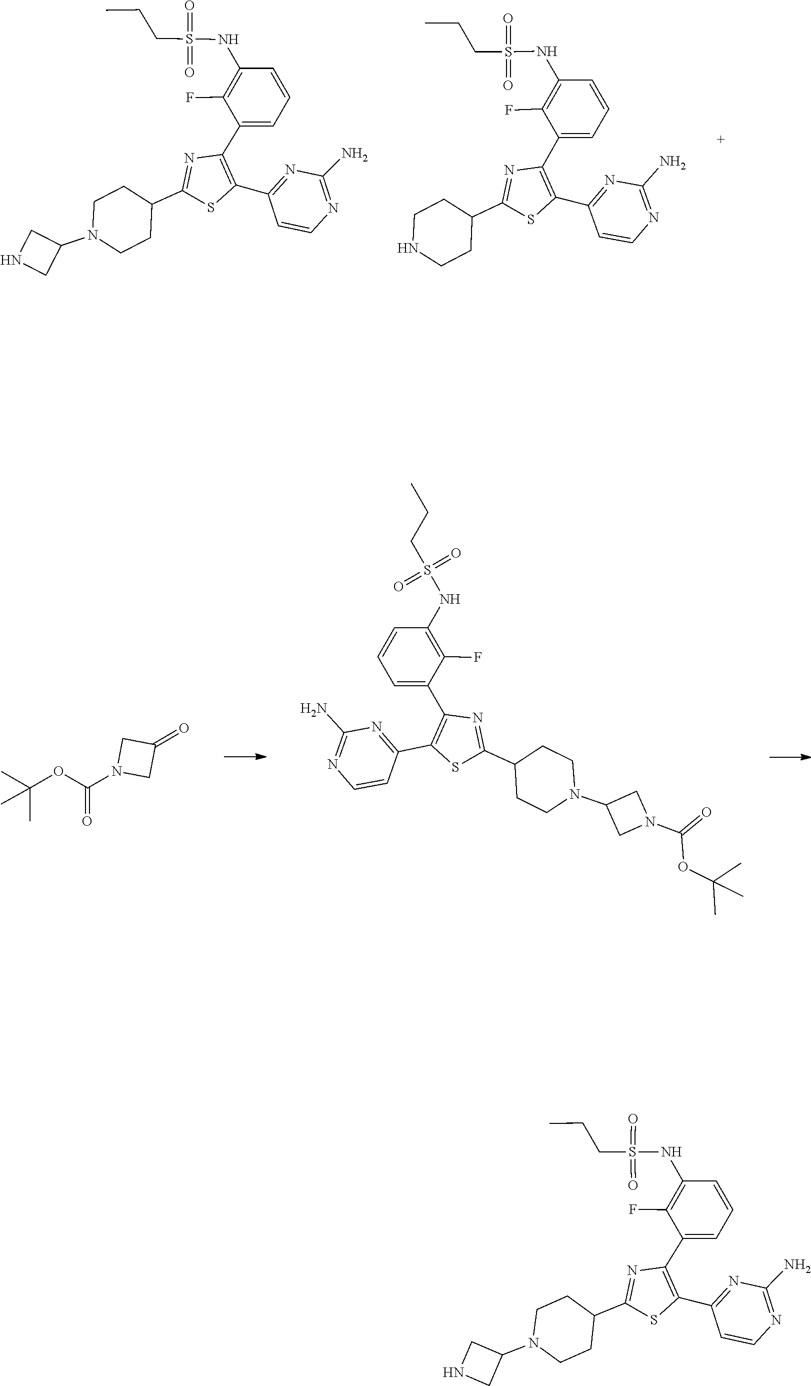 [SMILES: CCCS(=O)(=O)Nc1cccc(-c2nc(C3CCN(C4CNC4)CC3)sc2-c2ccnc(N)n2)c1F ; CCCS(=O)(=O)Nc1cccc(-c2nc(C3CCNCC3)sc2-c2ccnc(N)n2)c1F ; CC(C)(C)OC(=O)N1CC(=O)C1 ; CCCS(=O)(=O)Nc1cccc(-c2nc(C3CCN(C4CN(C(=O)OC(C)(C)C)C4)CC3)sc2-c2ccnc(N)n2)c1F ; CCCS(=O)(=O)Nc1cccc(-c2nc(C3CCN(C4CNC4)CC3)sc2-c2ccnc(N)n2)c1F]

### Step-1: tert-butyl 3-(4-(5-(2-aminopyrimidin-4-yl)-4-(2-fluoro-3-(propylsulfonamido)phenyl)thiazol-2-yl)piperidin-1-yl)azetidine-1-carboxylate

To a solution of N-{3-[5-(2-aminopyrimidin-4-yl)-2-(piperidin-4-yl)-1,3-thiazol-4-yl]-2-fluorophenyl}propane-1-sulfonamide (130.00 mg, 0.2728 mmol) and tert-butyl 3-oxoazetidine-1-carboxylate (46.70 mg, 0.2728 mmol) in DMSO (0.50 mL) and DCM (4.00 mL) was added triethylamine (37.81 μL, 27.60 mg, 0.2728 mmol) followed by sodium triacetoxyborohydride (173.43 mg, 0.8183 mmol) powder in portions. The reaction was stirred at room temperature for sixteen hours. The reaction was diluted with DCM (30 mL). The organic solution was washed with water (1 mL), dried over sodium sulfate, filtered, and concentrated in vacuo. The crude product was loaded onto a Redi-Sep prepacked silica gel column eluting with a gradient of 2-10% MeOH in DCM to provide tert-butyl 3-(4-(5-(2-aminopyrimidin-4-yl)-4-(2-fluoro-3-(propylsulfonamido)phenyl)thiazol-2-yl)piperidin-1-yl)azetidine-1-carboxylate (0.146 g, 84.72%). LCMS: C₂₉H₃₈FN₇O₄S₂ requires 631.24, found m/z=632.56 [M+H]⁺.

### Step-2: N-(3-(5-(2-aminopyrimidin-4-yl)-2-(1-(azetidin-3-yl)piperidin-4-yl)thiazol-4-yl)-2-fluorophenyl)propane-1-sulfonamide

To a solution of tert-butyl 3-(4-(5-(2-aminopyrimidin-4-yl)-4-(2-fluoro-3-(propylsulfonamido)phenyl)thiazol-2-yl)piperidin-1-yl)azetidine-1-carboxylate (146.00 mg, 0.2311 mmol) in DCM (2 mL) was added hydrogen chloride (2.00 mL, 0.29 g, 8.0000 mmol) in dioxane (4 M). The reaction was stirred for twenty minutes. Solvents were removed under reduced pressure to provide N-(3-(5-(2-aminopyrimidin-4-yl)-2-(1-(azetidin-3-yl)piperidin-4-yl)thiazol-4-yl)-2-fluorophenyl)propane-1-sulfonamide without further purification. LCMS C₂₄H₃₀FN₇O₂S₂ requires: 531.19; found: m/z=532.44 [M+H]⁺.

### General Procedure for Reductive Amination Followed by Deprotection

### Synthesis of N-(2-fluoro-3-(1-(4-(piperazin-1-ylmethyl)phenyl)-3-(pyridin-4-yl)-1H-pyrazol-4-yl)phenyl)propane-1-sulfonamide

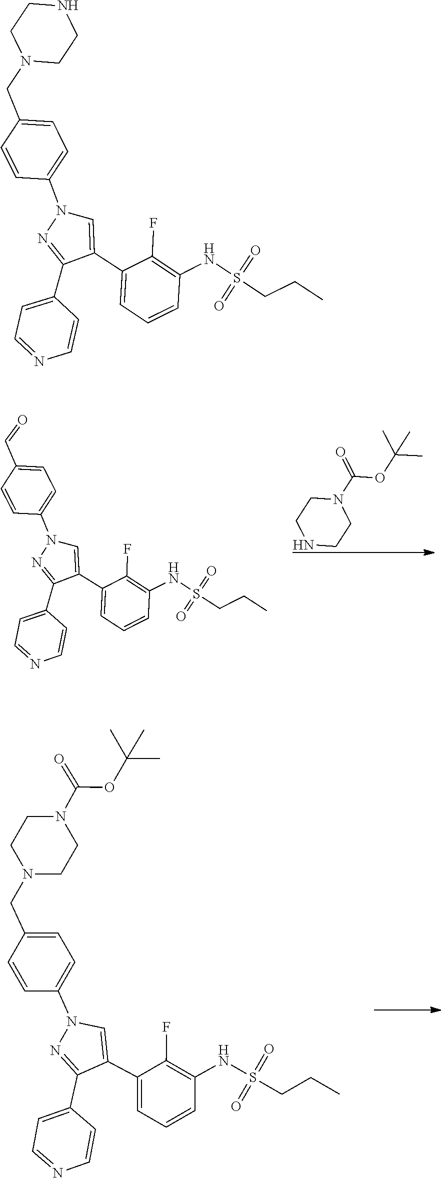 [SMILES: CCCS(=O)(=O)Nc1cccc(-c2cn(-c3ccc(CN4CCNCC4)cc3)nc2-c2ccncc2)c1F ; CCCS(=O)(=O)Nc1cccc(-c2cn(-c3ccc(C=O)cc3)nc2-c2ccncc2)c1F ; CC(C)(C)OC(=O)N1CCNCC1 ; CCCS(=O)(=O)Nc1cccc(-c2cn(-c3ccc(CN4CCN(C(=O)OC(C)(C)C)CC4)cc3)nc2-c2ccncc2)c1F]

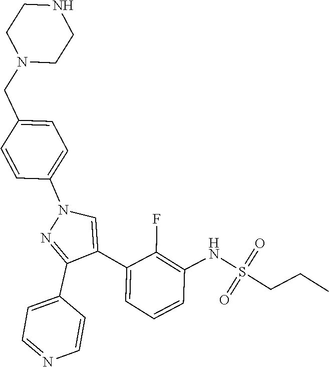 [SMILES: CCCS(=O)(=O)Nc1cccc(-c2cn(-c3ccc(CN4CCNCC4)cc3)nc2-c2ccncc2)c1F]

### Step-1: tert-butyl 4-(4-(4-(2-fluoro-3-(propylsulfonamido)phenyl)-3-(pyridin-4-yl)-1H-pyrazol-1-yl)benzyl)piperazine-1-carboxylate

To a solution of N-{2-fluoro-3-[1-(4-formylphenyl)-3-(pyridin-4-yl)pyrazol-4-yl]phenyl}propane-1-sulfonamide (101.20 mg, 0.2179 mmol) and tert-butyl piperazine-1-carboxylate (44.64 mg, 0.2396 mmol) in DCM (3.0 mL) was added acetic acid (0.05 mL) followed by the addition of sodium triacetoxyborohydride (138.52 mg, 0.6536 mmol). The reaction was stirred for thirty minutes; however, the starting material did not dissolve. Thus, MeOH (0.10 mL) was added and the starting material started to dissolve. The reaction was stirred for 2.5 hours. The crude product was diluted with DCM (30 mL), the solution was washed with water and brine, dried over sodium sulfate, filtered, and concentrated in vacuo. The crude product was loaded onto a Redi-Sep prepacked silica gel column eluting with a gradient of 20-100% EtOAc in DCM to afford tert-butyl 4-[(4-{4-[2-fluoro-3-(propane-1-sulfonamido)phenyl]-3-(pyridin-4-yl)pyrazol-1-yl}phenyl)methyl]piperazine-1-carboxylate (0.121 g, 70.00%). LCMS:C₃₃H₃₉FN₆O₄S requires: 634.3, found: m/z=635.7 [M+H]⁺.

### Step-2: N-(2-fluoro-3-(1-(4-(piperazin-1-ylmethyl)phenyl)-3-(pyridin-4-yl)-1H-pyrazol-4-yl)phenyl)propane-1-sulfonamide

To a solution of tert-butyl 4-[(4-{4-[2-fluoro-3-(propane-1-sulfonamido)phenyl]-3-(pyridin-4-yl)pyrazol-1-yl}phenyl)methyl]piperazine-1-carboxylate (121.00 mg, 0.1906 mmol) in DCM (4 mL) was added TFA (1 mL). The reaction was stirred for thirty minutes and then TFA and DCM were removed in vacuo. The crude product was loaded onto Redi-Sep prepacked C18 column (30 g) eluting with a gradient of 10-90% acetonitrile in water (with 0.01% TFA) to afford N-(2-fluoro-3-f{1-[4-(piperazin-1-ylmethyl)phenyl]-3-(pyridin-4-yl)pyrazol-4-yl}phenyl)propane-1-sulfonamide (0.1 g, 81.12%) as a TFA salt. LCMS: C₂₈H₃₁FN₆O₂S, requires: 534.22, found: m/z=535.57 [M+H]⁺.

The following scheme uses another modified General Procedure for Amide Coupling Followed by Deprotection.

### Synthesis of N-(3-(5-(2-aminopyrimidin-4-yl)-2-(1-(piperidine-4-carbonyl)piperidin-4-yl)thiazol-4-yl)-2-fluorophenyl)propane-1-sulfonamide

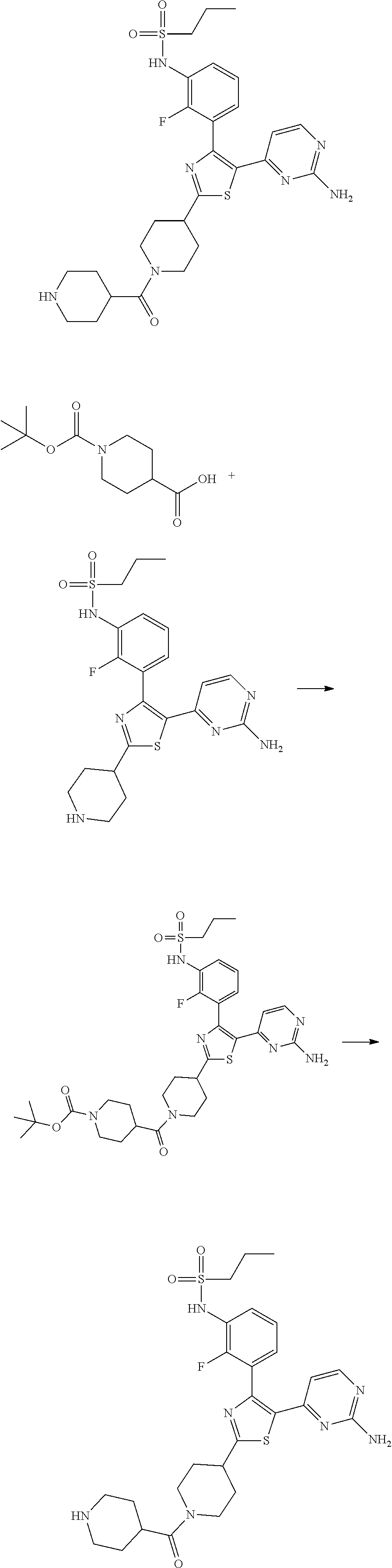

### Step-1: tert-butyl 4-(4-(5-(2-aminopyrimidin-4-yl)-4-(2-fluoro-3-(propylsulfonamido)phenyl)thiazol-2-yl)piperidine-1-carbonyl)piperidine-1-carboxylate

To a solution of 1-(tert-butoxycarbonyl)piperidine-4-carboxylic acid (81.78 mg, 0.3567 mmol) and [(dimethylamino)({[1,2,3]triazolo[4,5-b]pyridin-3-yloxy})methylidene]dimethylazanium; hexafluoro-lambda5-phosphanide (135.63 mg, 0.3567 mmol) in DMF (1 mL) was added N,N-diisopropylethylamine (256.12 μL, 184.41 mg, 1.4268 mmol). After stirring the reaction for three minutes, the reaction mixture was added into N-{3-[5-(2-aminopyrimidin-4-yl)-2-(piperidin-4-yl)-1,3-thiazol-4-yl]-2-fluorophenyl}propane-1-sulfonamide (170.00 mg, 0.3567 mmol) in DMF (0.5 mL) and the reaction mixture was stirred for twenty minutes. The crude product was diluted with EtOAc (10 mL), washed with water and brine, dried over sodium sulfate, filtered, and concentrated in vacuo. The crude product was loaded onto Redi-Sep prepacked silica gel column eluting with EtOAc in heptanes, to afford tert-butyl 4-{4-[5-(2-aminopyrimidin-4-yl)-4-[2-fluoro-3-(propane-1-sulfonamido)phenyl]-1,3-thiazol-2-yl]piperidine-1-carbonyl}piperidine-1-carboxylate (0.145 g, 38.41%). LCMS: C₃₂H₄₂FN₇O₅S₂ requires: 687.3, found: m/z=688.7 [M+H]⁺.

### Step-2: N-(3-(5-(2-aminopyrimidin-4-yl)-2-(1-(piperidine-4-carbonyl)piperidin-4-yl)thiazol-4-yl)-2-fluorophenyl)propane-1-sulfonamide

Tert-butyl 4-{4-[5-(2-aminopyrimidin-4-yl)-4-[2-fluoro-3-(propane-1-sulfonamido)phenyl]-1,3-thiazol-2-yl]piperidine-1-carbonyl}piperidine-1-carboxylate (145.00 mg, 0.2108 mmol) was dissolved in 50% v/v TFA in DCM (4 mL) and the solution was stirred for thirty minutes. TFA and DCM were then removed in vacuo. The crude product was loaded onto Redi-Sep prepacked C18 column (30 g) eluting with a gradient of 10-90% acetonitrile in water (0.01% TFA) to afford N-{3-[5-(2-aminopyrimidin-4-yl)-2-[1-(piperidine-4-carbonyl)piperidin-4-yl]-1,3-thiazol-4-yl]-2-fluorophenyl}propane-1-sulfonamide (0.1 g, 67.6%). LCMS: C₂₇H₃₄FN₇O₃S₂, requires: 587.2, found: m/z=588.6 [M+H]⁺. ¹H NMR (500 MHz, DMSO-d₆) δ 9.74 (s, 1H), 8.49 (d, J=11.1 Hz, 1H), 8.26 (s, 1H), 8.09 (d, J=5.3 Hz, 1H), 7.55 (t, J=7.7 Hz, 1H), 7.40 (t, J=7.0 Hz, 1H), 7.33 (t, J=7.9 Hz, 1H), 6.84 (s, 2H), 6.12 (d, J=5.2 Hz, 1H), 4.46 (d, J=12.9 Hz, 1H), 4.08 (d, J=13.5 Hz, 1H), 3.26 (dd, J=35.6, 12.8 Hz, 4H), 3.06 (dd, J=9.0, 6.3 Hz, 2H), 3.02-2.86 (m, 2H), 2.76 (d, J=19.8 Hz, 2H), 2.15 (dd, J=25.6, 12.8 Hz, 2H), 1.85-1.62 (m, 8H), 1.63-1.49 (m, 1H), 1.27 (q, J=5.8, 4.7 Hz, 2H), 0.93 (t, J=7.4 Hz, 3H).

### Synthesis of (R)—N-(5-chloro-2-fluoro-3-(1-(4-(3-methylpiperazin-1-yl)phenyl)-3-(pyridin-4-yl)-1H-pyrazol-4-yl)phenyl)propane-1-sulfonamide (12)

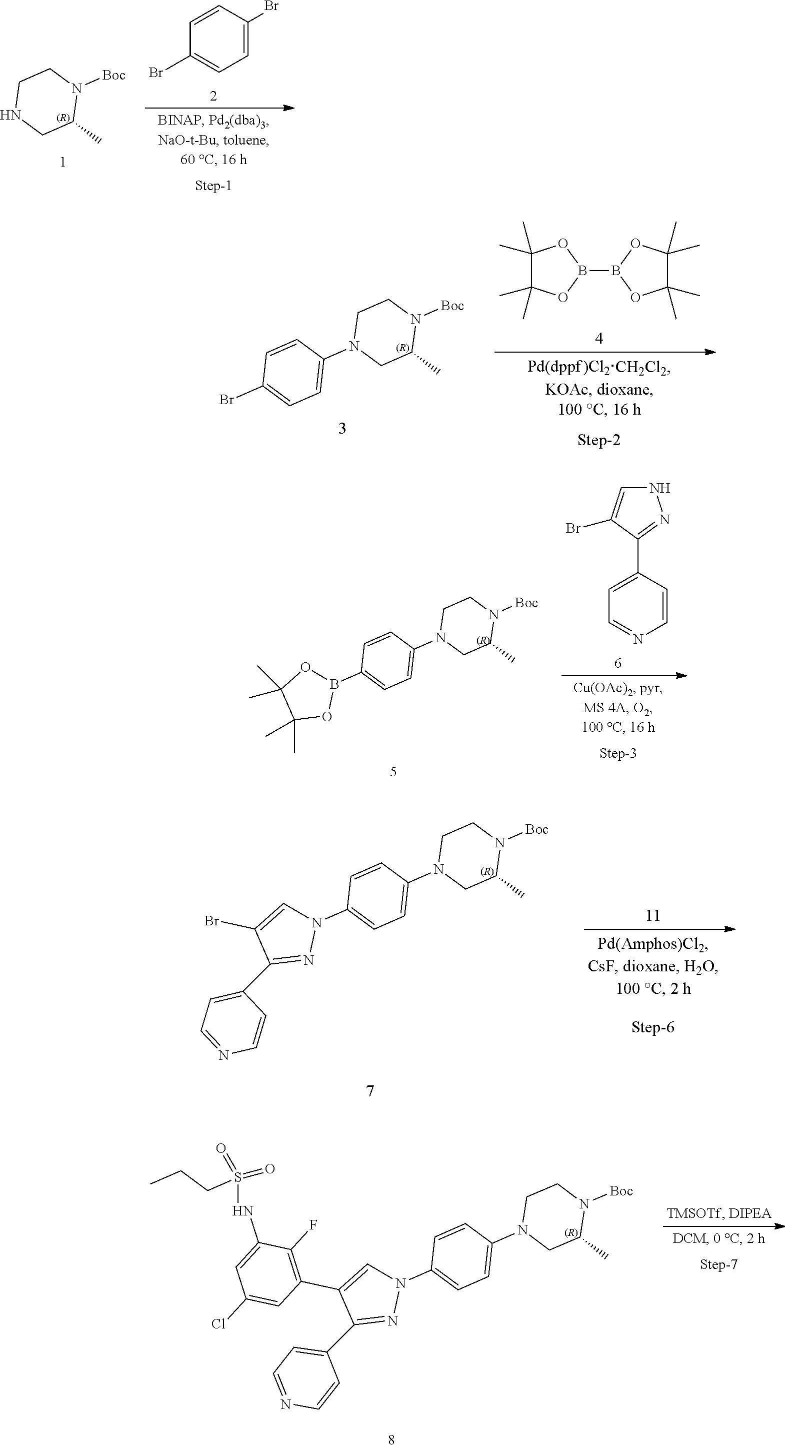

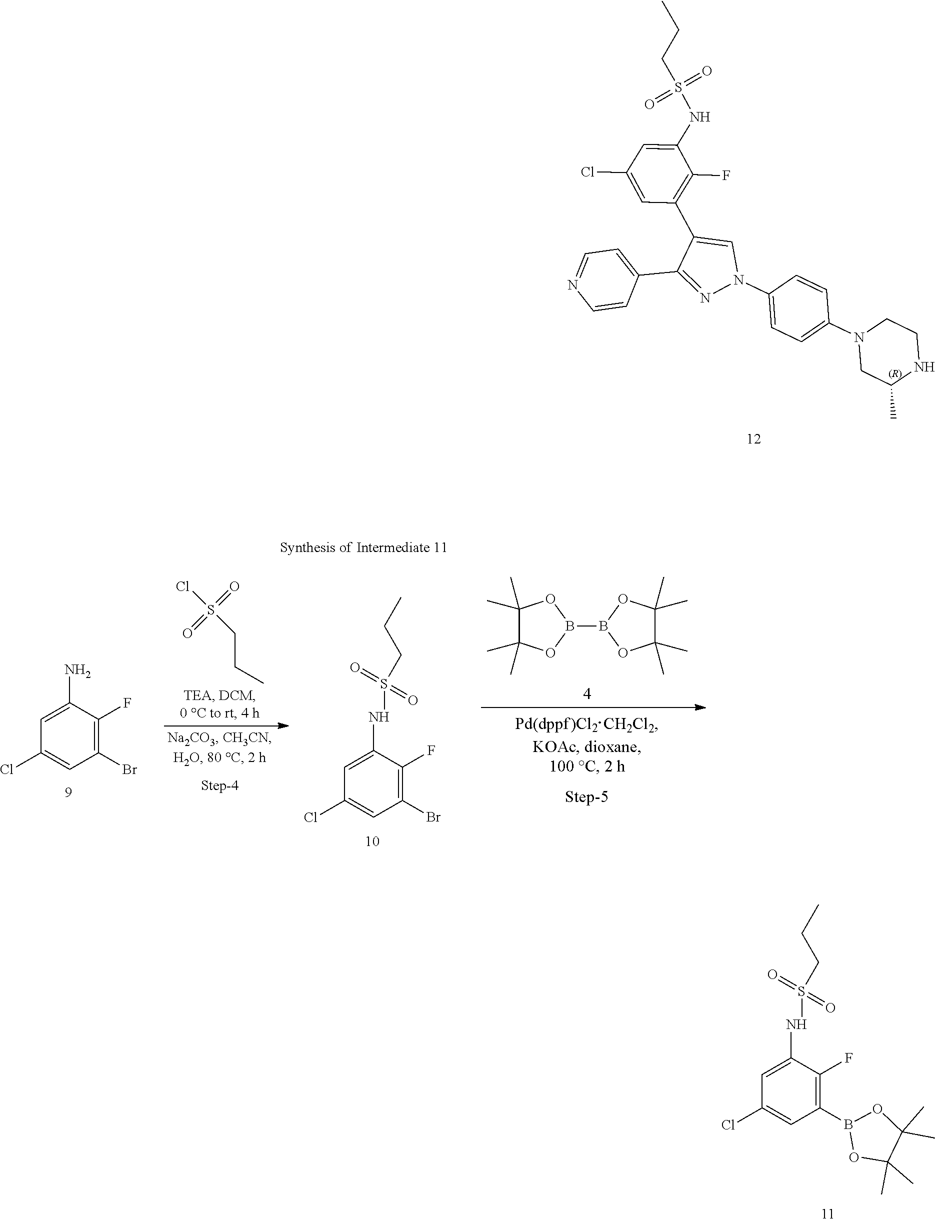

### Step-1: Synthesis of tert-butyl (R)-4-(4-bromophenyl)-2-methylpiperazine-1-carboxylate (3)

To a stirred solution of compound 1 (4.5 g, 22.47 mmol, 1.0 equiv) and compound 2 (12.25 g, 56.2 mmol, 2.5 equiv) in toluene (300 mL) was added sodium tert-butoxide (6.48 g, 67.4 mmol. 3.0 equiv) and BINAP (0.700 g, 1.123 mmol, 0.05 equiv) at room temperature. The resulting mixture was purged with N₂ for 10 min and then Pd₂(dba)₃ (1.029 g, 1.123 mmol, 0.05 equiv) was added. The resulting mixture was purged with N₂ for another 5 min and then heated at 60° C. for 16 h. The progress of the reaction was monitored by TLC (10% ethyl acetate:petroleum ether, R_{f}: ~0.8). The reaction mixture was then cooled to room temperature and filtered through a Celite pad and washed with ethyl acetate (100 mL). The filtrate was diluted with water (200 mL) and extracted with ethyl acetate (2×100 mL). The combined organic layer was dried over sodium sulphate and concentrated under vacuum to afford a crude compound (17 g) as a brown liquid. The crude compound was purified via Isolera (silica gel: 230-400 mesh) using ethyl acetate:petroleum ether (5-10%) as eluent to afford compound 3 (6.5 g, 78%) as a pale yellow solid.

### Step-2: Synthesis of tert-butyl (R)-2-methyl-4-(4-(4,4,5,5-tetramethyl-1,3,2-dioxaborolan-2-yl)phenyl)piperazine-1-carboxylate (5)

To a solution of compound 3 (6.5 g, 18.30 mmol, 1.0 equiv) and compound 4 (6.97 g, 27.4 mmol, 1.5 equiv) in 1,4-dioxane (300 mL) was added potassium acetate (3.59 g, 36.6 mmol, 2.0 equiv) at room temperature. The resulting mixture was purged with N₂ for 10 min and then PdCl₂(dppf) CH₂Cl₂ adduct (1.494 g, 1.830 mmol, 0.1 equiv) was added. The resulting mixture was purged with N₂ for another 5 min and then heated at 100° C. for 16 h. The progress of the reaction was monitored by TLC (10% ethyl acetate:petroleum ether, R_{f}: ~0.6). The reaction mixture was then cooled to room temperature and filtered through a Celite pad and washed with ethyl acetate (100 mL). The filtrate was diluted with water (200 mL) and extracted with ethyl acetate (2×100 mL). The combined organic layer was dried over sodium sulphate and concentrated under vacuum to afford a crude compound (14 g) as a brown liquid.

The crude compound was purified via Isolera (silica gel: 230-400 mesh) using ethyl acetate:petroleum ether (5-7%) as eluent to afford compound 5 (7 g, 92%) as a pale yellow solid.

### Step-3: Synthesis of tert-butyl (R)-4-(4-(4-bromo-3-(pyridin-4-yl)-1H-pyrazol-1-yl)phenyl)-2-methylpiperazine-1-carboxylate (7)

To a mixture of compound 6 (1.5 g, 6.69 mmol, 1.0 equiv) and compound 5 (3.23 g, 8.03 mmol, 1.2 equiv) in pyridine (20 mL) was added molecular sieves, 4 Å (0.731 g, 3.35 mmol, 0.5 equiv) and copper (II) acetate (1.824 g, 10.04 mmol, 1.5 equiv) at room temperature. The resulting mixture was then purged with O₂ for 15 min and then heated at 100° C. for 16 h. The progress of the reaction was monitored by TLC (50% ethyl acetate:petroleum ether, R_{f}: ~0.3) and LCMS. The reaction mixture was then cooled to room temperature, filtered through a Celite pad, and washed with ethyl acetate (100 mL). The filtrate was diluted with water (200 mL) and extracted with ethyl acetate (2×100 mL). The combined organic layer was dried over sodium sulphate and concentrated under vacuum to afford a crude compound (10 g) as a brown liquid. The crude compound was purified via Isolera (silica gel: 230-400 mesh) using ethyl acetate:petroleum ether (10%) as eluent to afford compound 7 (1.7 g, 47.6%) as a yellow gummy liquid.

### Step-4: Synthesis of N-(3-bromo-5-chloro-2-fluorophenyl)propane-1-sulfonamide (10)

To a stirred solution of compound 9 (1.0 g, 4.46 mmol, 1.0 equiv) in dichloromethane (30 mL) was added triethylamine (2.484 mL, 17.82 mmol, 4.0 equiv) and propane-1-sulfonyl chloride (1.003 mL, 8.91 mmol, 2.0 equiv) at 0° C., and the resulting reaction mixture was then warmed and stirred at room temperature for 4 h. Upon completion of the reaction as confirmed by TLC (10% ethyl acetate:petroleum ether, R_{f}: ~0.1) the reaction mixture was then concentrated under vacuum to give the crude product which was dissolved in acetonitrile (30 mL). To this was added Na₂CO₃ (4.72 g, 44.6 mmol) in water (30 mL) and the reaction was heated at 80° C. for 2 h. Then the reaction mixture was concentrated under vacuum and the residue obtained was acidified with aq. citric acid solution. The solid obtained was filtered and dried to give the crude product which was purified via Isolera (silica gel, 230-400 mesh) using ethyl acetate:petroleum ether (35-40%) as eluent. The pure fractions were concentrated under vacuum and the solid obtained was washed with petroleum ether (100 mL) and dried under vacuum to give compound 10 (1.0 g, 63.8%) as a white solid.

### Step-5: Synthesis of N-(5-chloro-2-fluoro-3-(4,4,5,5-tetramethyl-1,3,2-dioxaborolan-2-yl)phenyl)propane-1-sulfonamide (11)

To a mixture of compound 10 (1.0 g, 3.02 mmol, 1.0 equiv) and compound 4 (1.152 g, 4.54 mmol, 1.5 equiv) in 1,4-dioxane (15 mL) was added potassium acetate (1.039 g, 10.59 mmol, 3.5 equiv) and PdCl₂(dppf) CH₂Cl₂ (0.247 g, 0.302 mmol, 0.1 equiv). The resulting mixture was purged with nitrogen for 10 min and then stirred at 100° C. for 2 h. Upon completion of the reaction, as confirmed by LCMS, the reaction mixture was filtered through a Celite bed and washed with 1,4-dioxane (25 mL). The filtrate was concentrated under vacuum to afford crude compound 11. LCMS: m/z: 294 indicated the corresponding boronic acid of 11.

### Step-6: Synthesis of tert-butyl (R)-4-(4-(4-(5-chloro-2-fluoro-3-(propylsulfonamido)phenyl)-3-(pyridin-4-yl)-1H-pyrazol-1-yl)phenyl)-2-methylpiperazine-1-carboxylate (8)

To a stirred solution of compound 7 (1 g, 2.006 mmol, 1.0 equiv) and compound 11 (3 g, crude) in 1,4-dioxane (15 mL) and water (4 mL) was added CsF (0.610 g, 4.01 mmol, 2.0 equiv) and Pd(amphos)Cl₂ (0.142 g, 0.201 mmol, 0.1 equiv) and the reaction mixture was purged with nitrogen for 10 min. The reaction was then heated at 100° C. for 2 h. Upon completion of reaction as confirmed by LCMS, the reaction mixture was filtered through a Celite bed and washed with 1,4-dioxane (20 mL). The filtrate was concentrated under vacuum and the residue was washed with water (100 mL) and extracted with ethyl acetate (100 mL). The combined organic layer was dried over sodium sulfate, filtered, and concentrated under vacuum to provide the crude product which was purified via Isolera (silica gel, 230-400 mesh) using ethyl acetate:petroleum ether (30-35%) as eluent to afford compound 8 (0.480 g, 30%) as a yellow solid.

### Step-7: Synthesis of (R)—N-(5-chloro-2-fluoro-3-(1-(4-(3-methylpiperazin-1-yl)phenyl)-3-(pyridin-4-yl)-1H-pyrazol-4-yl)phenyl)propane-1-sulfonamide (12)

To stirred solution of compound 8 (0.480 g, 0.717 mmol, 1.0 equiv) in DCM (10 mL) was added DIPEA (0.260 mL, 1.435 mmol, 2.0 equiv) and TMS-OTf (0.195 mL, 1.076 mmol, 1.5 equiv) at 0° C. and the reaction was stirred at room temperature for 2 h. Upon completion of reaction as confirmed by LCMS, the reaction mixture was quenched with water (20 mL) and extracted with DCM (2×20 mL). The combined organic layer was dried over Na₂SO₄ and concentrated under vacuum provide the crude product which was purified by prep-HPLC (Column: X Bridge C8 150, Method: Formic acid: ACN, Rₜ: 10 min, Flow rate: 15 mL/min) to provide 12 (0.222 g, 51.6%, Formate salt) as a yellow solid. LCMS: m/z: 569.2 (M+H)⁺, Rₜ: 1.329 min, Area: 95.991%; Column: X-BRIDGE C8 (50×4.6 mm) 3.5 m, Mobile phase: A: 0.1% TFA in H₂O, B: ACN, Flow Rate: 2.0 mL/min. HPLC: Rₜ: 7.636 min, Area: 94.819%, Column: Atlantis T3 (150×4.6) mm, 3 m, Mobile phase: A:0.1% TFA in water, Mobile phase: B: ACN, Flow: 1.2 mL/min. ¹H NMR (400 MHz, DMSO-d₆): δ 8.78 (s, 1H), 8.57 (q, J=1.60 Hz, 2H), 8.15 (s, 1H), 7.83 (d, J=9.20 Hz, 2H), 7.43-7.50 (m, 4H), 7.18 (d, J=9.20 Hz, 2H), 3.85 (q, J=13.20 Hz, 2H), 3.40-3.33 (m, 3H), 3.14-3.06 (m, 3H), 2.94 (m, 1H), 2.74-2.68 (m, 1H), 1.69-1.63 (m, 2H), 1.26 (d, J=6.40 Hz, 3H), 0.92 (t, J=7.20 Hz, 3H).

### Synthesis of (S)—N-(5-chloro-2-fluoro-3-(1-(4-(3-methylpiperazin-1-yl)phenyl)-3-(pyridin-4-yl)-1H-pyrazol-4-yl)phenyl)propane-1-sulfonamide (12)

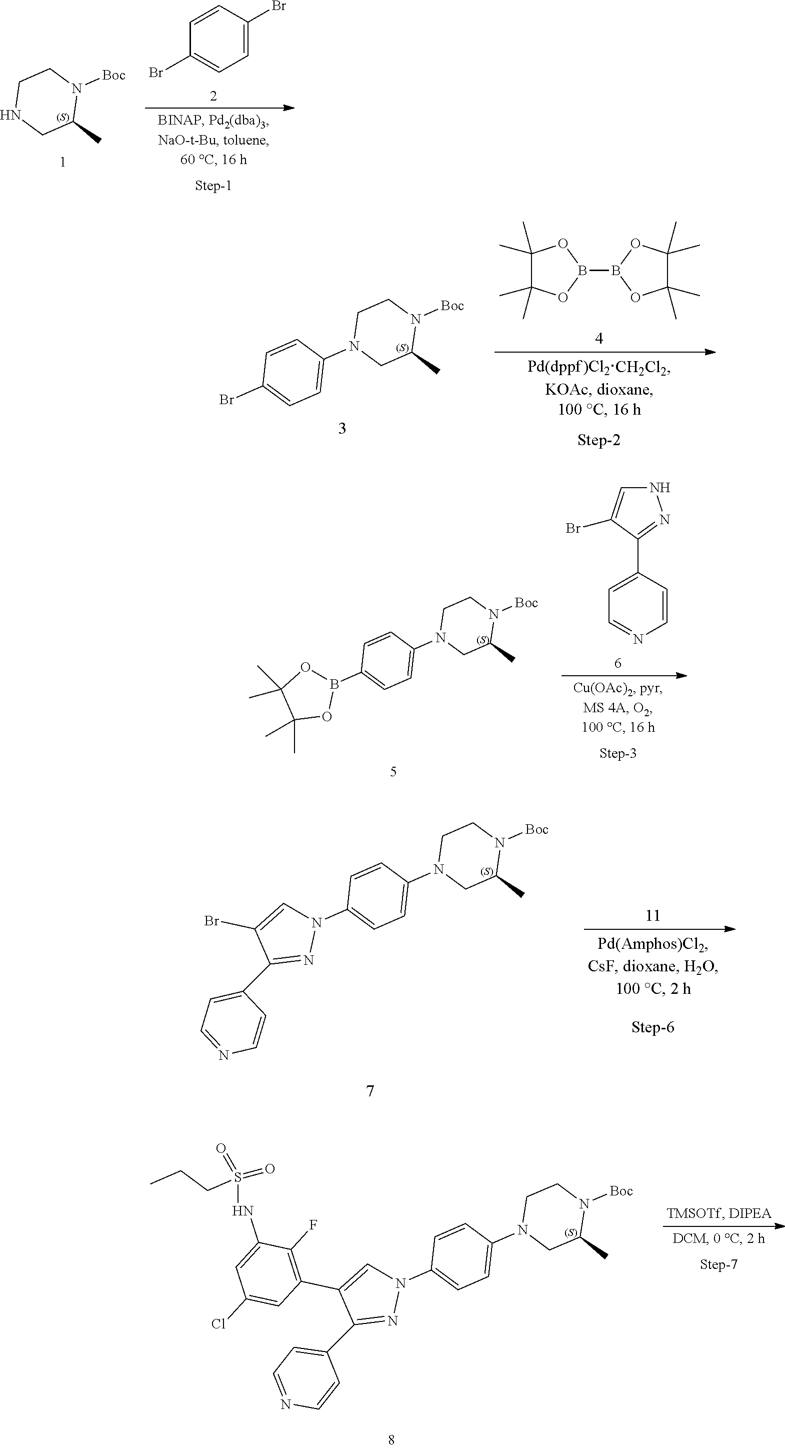

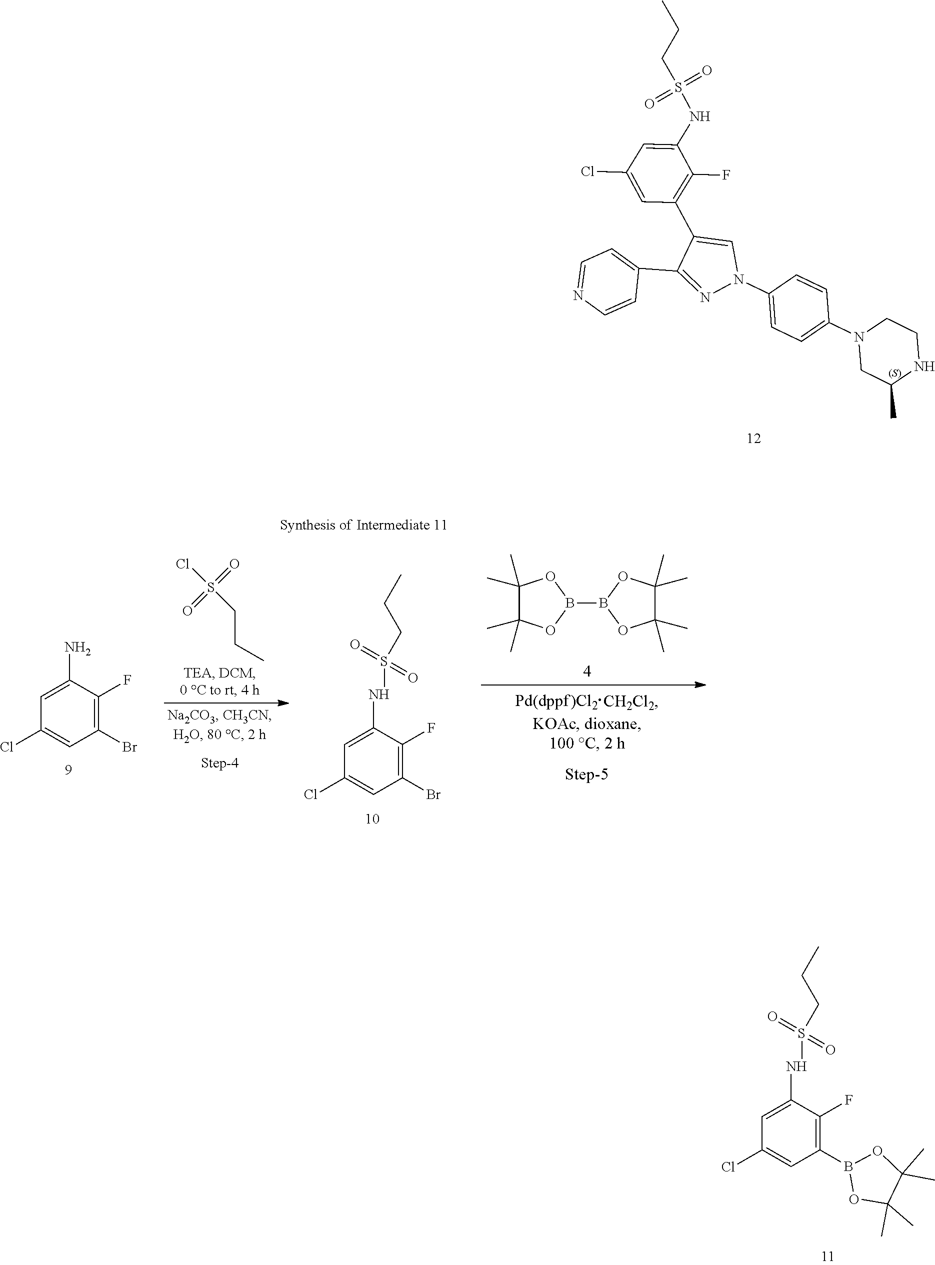

### Step-1: Synthesis of tert-butyl (S)-4-(4-bromophenyl)-2-methylpiperazine-1-carboxylate (3)

To a stirred solution of compound 1 (3.5 g, 17.48 mmol, 1.0 equiv) and compound 2 (10.31 g, 43.7 mmol, 2.5 equiv) in toluene (300 mL) was added sodium tert-butoxide (5.04 g, 52.4 mmol. 3.0 equiv) and BINAP (0.544 g, 0.874 mmol, 0.05 equiv) at room temperature. The resulting mixture was purged with N₂ for 10 min and then Pd₂(dba)₃ (0.800 g, 0.874 mmol, 0.05 equiv) was added. The resulting mixture was then purged with N₂ for another 5 min and the reaction was then heated at 60° C. for 16 h. The progress of the reaction was monitored by TLC (10% ethyl acetate:petroleum ether, R_{f}: ~0.8). The reaction mixture was then cooled to room temperature and filtered through a Celite pad and washed with ethyl acetate (100 mL). The filtrate was diluted with water (200 mL) and extracted with ethyl acetate (2×100 mL). The combined organic layer was dried over sodium sulphate and concentrated under vacuum to afford a crude compound (15 g) as a brown liquid. The crude compound was purified via Isolera (silica gel: 230-400 mesh) using ethyl acetate:petroleum ether (5-10%) as eluent to afford compound 3 (6.2 g, 95%) as a pale yellow solid.

### Step-2: Synthesis of tert-butyl (S)-2-methyl-4-(4-(4,4,5,5-tetramethyl-1,3,2-dioxaborolan-2-yl)phenyl)piperazine-1-carboxylate (5)

To a solution of compound 3 (7.7 g, 21.67 mmol, 1.0 equiv) and compound 4 (8.26 g, 32.5 mmol, 1.5 equiv) in 1,4-dioxane (300 mL) was added potassium acetate (4.25 g, 43.3 mmol, 2.0 equiv) at room temperature. The resulting mixture was purged with N₂ for 10 min and then PdCl₂(dppf) CH₂Cl₂ adduct (1.770 g, 2.167 mmol, 0.1 equiv) was added. The resulting mixture was purged with N₂ for another 5 min and the reaction was then heated at 100° C. for 16 h. The progress of the reaction was monitored by TLC (10% ethyl acetate:petroleum ether, R_{f}: ~0.6). The reaction mixture was then cooled to room temperature and filtered through a Celite pad and washed with ethyl acetate (100 mL). The filtrate was diluted with water (200 mL) and extracted with ethyl acetate (2×100 mL). The combined organic layer was dried over sodium sulphate and concentrated under vacuum to afford a crude compound (16 g) as a brown liquid. The crude compound was purified via Isolera (silica gel: 230-400 mesh) using ethyl acetate:petroleum ether (5-7%) as eluent to afford compound 5 (8.7 g, 98%) as a pale yellow solid.

### Step-3: Synthesis of tert-butyl (S)-4-(4-(4-bromo-3-(pyridin-4-yl)-1H-pyrazol-1-yl)phenyl)-2-methylpiperazine-1-carboxylate (7)

To a mixture of compound 6 (2 g, 8.93 mmol, 1.0 equiv) and compound 5 (4.31 g, 10.71 mmol, 1.2 equiv) in pyridine (25 mL) was added molecular sieves, 4 Å (0.974 g, 4.46 mmol, 0.5 equiv) and copper (II) acetate (2.432 g, 13.39 mmol, 1.5 equiv) at room temperature. The resulting mixture was purged with O₂ for 15 min and then the reaction was heated at 100° C. for 16 h. The progress of the reaction was monitored by TLC (50% ethyl acetate:petroleum ether, R_{f}: ~0.3) and LCMS. The reaction mixture was then cooled to room temperature and filtered through a Celite pad and washed with ethyl acetate (100 mL). The filtrate was diluted with water (200 mL) and extracted with ethyl acetate (2×100 mL). The combined organic layer was dried over sodium sulphate and concentrated under vacuum to afford a crude compound (12 g) as a brown liquid. The crude compound was purified via Isolera (silica gel: 230-400 mesh) using ethyl acetate:petroleum ether (15-20%) as eluent to afford compound 7 (1.5 g, 32%) as a yellow gummy liquid.

### Step-4: Synthesis of N-(3-bromo-5-chloro-2-fluorophenyl)propane-1-sulfonamide (10)

To a stirred solution of compound 9 (0.8 g, 3.56 mmol, 1.0 equiv) in dichloromethane (30 mL) was added triethylamine (1.44 g, 14.26 mmol, 4.0 equiv) and propane-1-sulfonyl chloride (1.203 mL, 10.69 mmol, 3.0 equiv) at 0° C. and the resulting reaction mixture was stirred at room temperature for 4 h. Upon completion of the reaction as confirmed by TLC (10% ethyl acetate:petroleum ether, R_{f}: ~0.1), the reaction mixture was then concentrated under vacuum to give the crude product which was dissolved in acetonitrile (30 mL). Na₂CO₃ (3.77 g, 35.6 mmol) in water (30 mL) was then added and the reaction was then heated at 80° C. for 2 h. Then the reaction mixture was concentrated under vacuum and the residue obtained was acidified with aq. citric acid solution. The solid obtained was filtered and dried to give the crude product which was purified via Isolera (silica gel, 230-400 mesh) using ethyl acetate:petroleum ether (35-40%) as eluent. The pure fractions were concentrated under vacuum and the solid obtained was washed with petroleum ether (25 mL) and dried under vacuum to give compound 10 (1.1 g, 91%) as a white solid.

### Step-5: Synthesis of N-(5-chloro-2-fluoro-3-(4,4,5,5-tetramethyl-1,3,2-dioxaborolan-2-yl)phenyl)propane-1-sulfonamide (11)

To a mixture of compound 10 (1.1 g, 3.33 mmol, 1.0 equiv) and compound 4 (1.267 g, 4.99 mmol, 1.5 equiv) in 1,4-dioxane (15 mL) was added potassium acetate (1.14 g, 11.65 mmol, 3.5 equiv) and PdCl₂(dppf) CH₂Cl₂ (0.272 g, 0.333 mmol, 0.1 equiv). The resulting mixture was purged with nitrogen for 10 min and then stirred at 100° C. for 2 h. Upon completion of the reaction, as confirmed by LCMS, the reaction mixture was filtered through a Celite bed and washed with 1,4-dioxane (25 mL). The filtrate was concentrated under vacuum to afford crude compound 11 (2.9 g, quant.). LCMS: m/z=294 corresponds to the boronic acid derivative of 11.

### Step-6: Synthesis of tert-butyl (S)-4-(4-(4-(5-chloro-2-fluoro-3-(propylsulfonamido)phenyl)-3-(pyridin-4-yl)-1H-pyrazol-1-yl)phenyl)-2-methylpiperazine-1-carboxylate (8)

To a stirred solution of compound 7 (1.1 g, 2.207 mmol, 1.0 equiv) and compound 11 (2.9 g, crude) in 1,4-dioxane (15 mL) and water (4 mL) was added CsF (0.670 g, 4.41 mmol, 2.0 equiv) and Pd(amphos)Cl₂ (0.156 g, 0.221 mmol, 0.1 equiv) and the reaction mixture was purged with nitrogen for 10 min. The reaction was then heated at 100° C. for 2 h. Upon completion of the reaction, as confirmed by LCMS, the reaction mixture was then filtered through a Celite bed and washed with 1,4-dioxane (20 mL). The filtrate was concentrated under vacuum and the residue was washed with water (100 mL) and extracted with ethyl acetate (100 mL). The combined organic layer was dried over sodium sulfate, filtered, and concentrated under vacuum to provide the crude product which was purified via Isolera (silica gel, 230-400 mesh) using ethyl acetate:petroleum ether (30-35%) as eluent to afford compound 8 (0.580 g, 31.8%) as a yellow solid.

### Step-7: Synthesis of (S)—N-(5-chloro-2-fluoro-3-(1-(4-(3-methylpiperazin-1-yl)phenyl)-3-(pyridin-4-yl)-1H-pyrazol-4-yl)phenyl)propane-1-sulfonamide (12)

To stirred solution of compound 8 (0.580 g, 0.867 mmol, 1.0 equiv) in DCM (10 mL) was added DIPEA (0.302 mL, 1.733 mmol, 2.0 equiv) and TMS-OTf (0.473 mL, 2.60 mmol, 3 equiv) at 0° C. and the reaction was then stirred at room temperature for 2 h. Upon completion of then reaction, as confirmed by LCMS, the reaction mixture was then quenched with water (20 mL) and extracted with DCM (2×20 mL). The combined organic layer was dried over Na₂SO₄ and concentrated under vacuum provide the crude which was purified by prep-HPLC (Column: Xselect C18 250 mm, Method: Formic acid (FA): ACN, Rₜ: 10 min, Flow rate: 15 mL/min) to provide 12 (0.156 g, 31.2%, formate salt) as a yellow solid. LCMS: Rₜ: 1.461 min, Area: 99.33%, m/z=569.2 (M+H)⁺. Column: BEH C18 (50×2.1 mm) 1.7 m; Mobile phase: A: 0.1% HCOOH in H₂O, B: ACN; Flow Rate: 0.8 mL/min. HPLC: Rₜ: 7.653 min, Area: 99.93%; Method Info: Column: Atlantis T3 (150×4.6 mm, 3 m; Mobile phase: A: 0.1% TFA in water; Mobile phase: B: ACN; Flow: 1.2 mL/min. ¹H NMR (400 MHz, DMSO-d₆): δ 9.00 (bs, 2H), 8.78 (s, 1H), 8.57 (q, J=1.60 Hz, 2H), 7.83 (d, J=9.20 Hz, 2H), 7.51-7.41 (m, 4H), 7.18 (d, J=9.20 Hz, 2H), 3.88-3.79 (m, 2H), 3.39-3.33 (m, 2H), 3.13-3.05 (m, 3H), 2.96-2.93 (m, 1H), 2.73-2.68 (m, 1H), 1.69-1.63 (m, 2H), 1.26 (d, J=6.80 Hz, 3H), 0.92 (t, J=7.60 Hz, 3H).

### Synthesis of (R)—N-(5-chloro-2-fluoro-3-(1-(5-(piperazin-1-yl)pyridin-2-yl)-3-(pyridin-4-yl)-1H-pyrazol-4-yl)phenyl)-3-fluoropyrrolidine-1-sulfonamide (11)

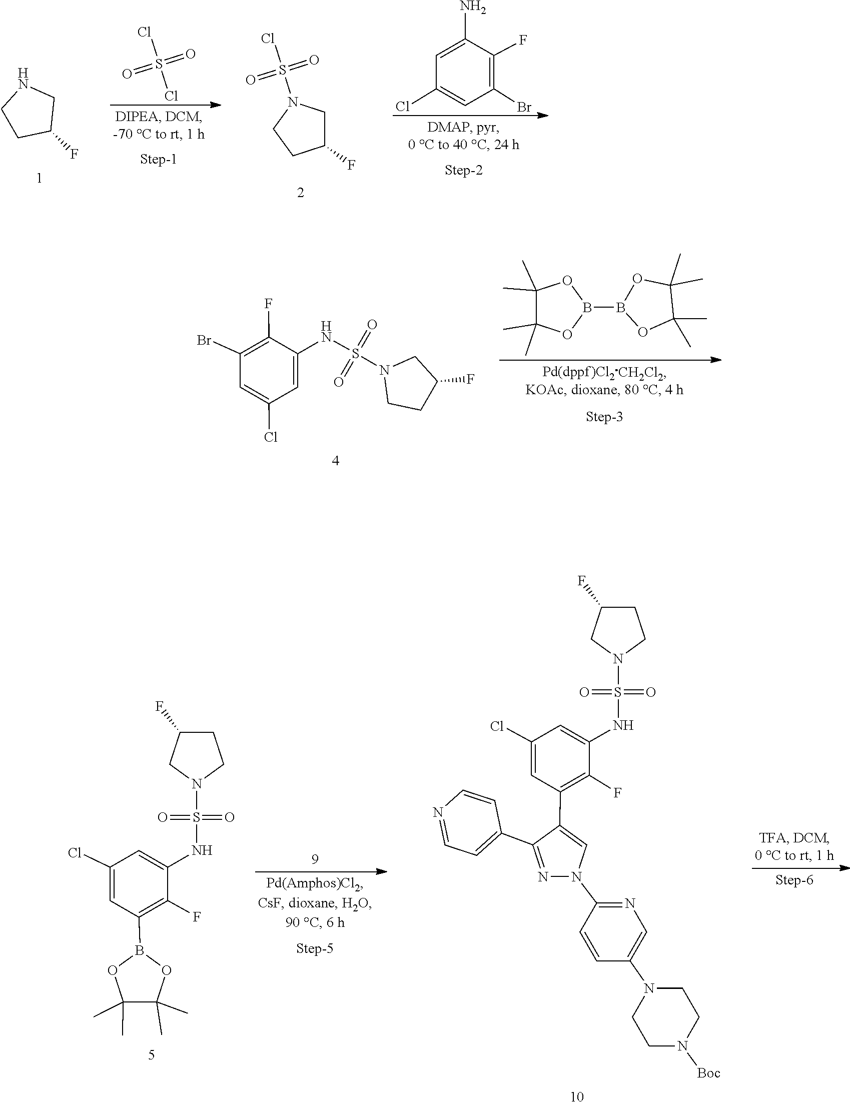

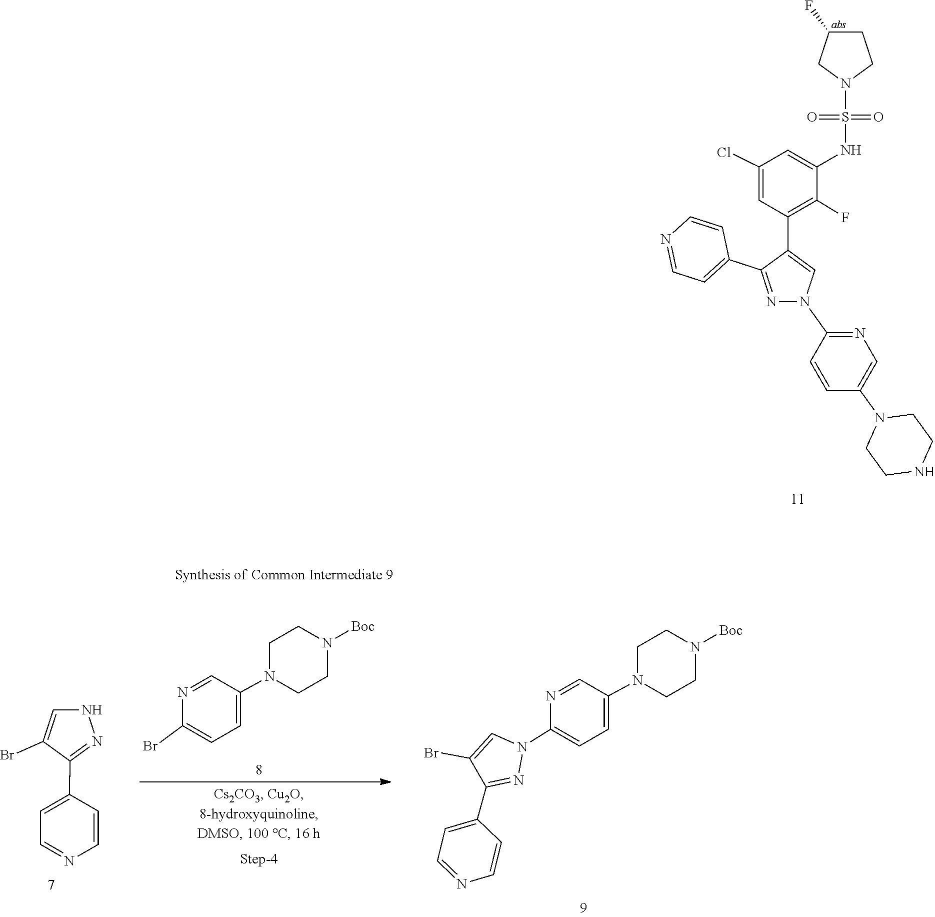 [SMILES: O=S(=O)(Nc1cc(Cl)cc(-c2cn(-c3ccc(N4CCNCC4)cn3)nc2-c2ccncc2)c1F)N1CC[C@@H](F)C1 ; Brc1c[nH]nc1-c1ccncc1 ; CC(C)(C)OC(=O)N1CCN(c2ccc(Br)nc2)CC1 ; CC(C)(C)OC(=O)N1CCN(c2ccc(-n3cc(Br)c(-c4ccncc4)n3)nc2)CC1]

### Step-1: Synthesis of (R)-3-fluoropyrrolidine-1-sulfonyl chloride (2)

To a solution of compound 1 (4.5 g, 35.8 mmol, 1.0 equiv) in DCM (90 mL) was added DIPEA (17.78 mL, 108 mmol, 3.0 equiv) and the solution was cooled to −70° C.

Sulfuryl dichloride (9.67 g, 71.7 mmol, 2.0 equiv) was then added and the reaction was stirred at −70° C. for one hour. The reaction was then warmed and stirred at room temperature for one hour. The progress of the reaction was monitored by TLC (20% ethyl acetate:petroleum ether, R_{f}: ~0.6, KMnO4 active). The reaction mixture was then quenched with water (200 mL), extracted with DCM (2×200 mL), and washed with 1.5N HCl (100 mL). The combined organic layer was dried over Na₂SO₄ and concentrated under vacuum at room temperature to afford compound 2 (5 g, 74.4%) as an off white solid.

### Step-2: Synthesis of (R)—N-(3-bromo-5-chloro-2-fluorophenyl)-3-fluoropyrrolidine-1-sulfonamide (4)

To a solution of compound 2 (3 g, 13.38 mmol, 1.0 equiv) in pyridine (4.5 mL) was added DMAP (0.327 g, 2.637 mmol, 0.2 equiv) followed by compound 3 (3.261 g, 17.37 mmol, 1.3 equiv) at 0° C., and the resulting reaction mixture was then heated to 40° C. for 24 h. Upon completion of the reaction, as confirmed by LCMS, the reaction mixture was concentrated under vacuum to afford a crude compound (5 g) as a brown gum. The crude compound was purified via Isolera (silica gel: 230-400 mesh) using ethyl acetate:petroleum ether (10-12%) as eluent to afford compound 4 (1.7 g, 33.8%) as a yellow solid.

### Step-3: Synthesis of (R)—N-(5-chloro-2-fluoro-3-(4,4,5,5-tetramethyl-1,3,2-dioxaborolan-2-yl)phenyl)-3-fluoropyrrolidine-1-sulfonamide (6)

To a solution of compound 4 (1 g, 2.66 mmol, 1.0 equiv) in 1,4-dioxane (30 mL) was added compound 5 (1.014 g, 3.99 mmol, 1.5 equiv) and potassium acetate (0.523 g, 5.32 mmol, 2.0 equiv) and the mixture was purged with nitrogen for 15 min. Then PdCl₂(dppf) DCM (0.195 g, 0.266 mmol, 0.1 equiv) was added and the reaction was heated to 90° C. for 4 h. The progress of the reaction was monitored by TLC (50% ethyl acetate:petroleum ether, R_{f}: ~0.4). The reaction mixture was then cooled to room temperature and filtered through a Celite pad and washed with ethyl acetate (100 mL). The filtrate was diluted with water (200 mL) and extracted with ethyl acetate (2×100 mL). The combined organic layer was passed through silica gel (100-120 mesh), dried over Na₂SO₄, and concentrated under vacuum to afford crude compound 6 (1.0 g, 89%) as a brown liquid.

### Step-4: Synthesis of tert-butyl 4-(6-(4-bromo-3-(pyridin-4-yl)-1H-pyrazol-1-yl)pyridin-3-yl)piperazine-1-carboxylate (9)

A mixture of compound 7 (0.5 g, 0.0022 mmol, 1.0 equiv) and 8 (0.75 g, 0.0022 mmol, 1.0 equiv) in DMSO (5 mL) was treated with cesium carbonate (1.45 g, 0.0044 mmol, 2.0 equiv), copper(I)oxide (0.032 g, 0.0002 mmol, 0.1 equiv), and 8-hydroxyquinoline (0.064 g, 0.0004 mmol, 0.2 equiv) at room temperature. The reaction mixture was purged with N₂ for 10 min and then heated at 100° C. in a closed vial for 16 h. Similarly, five additional batches were performed on 0.5 g scale.

All the batches were then cooled to room temperature, mixed together, and filtered through a Celite pad. The Celite pad was washed with ethyl acetate (100 mL). The filtrate was diluted with water (200 mL) and extracted with ethyl acetate (2×100 mL). The combined organic layer was dried over Na₂SO₄ and concentrated under vacuum to afford a crude compound (6 g) as a brown liquid. The crude compound was purified via Isolera (silica gel: 230-400 mesh) using ethyl acetate:petroleum ether (30-35%) as eluent to afford compound 9 (3.0 g, 46%) as an off-white solid.

### Step-5: Synthesis of tert-butyl (R)-4-(6-(4-(5-chloro-2-fluoro-3-((3-fluoropyrrolidine)-1-sulfonamido)phenyl)-3-(pyridin-4-yl)-1H-pyrazol-1-yl)pyridin-3-yl)piperazine-1-carboxylate (10)

To a mixture of compound 6 (1.0 g, 2.366 mmol, 1.0 equiv) and compound 9 (0.918 g, 1.892 mmol, 0.8 equiv) in 1,4-dioxane (24 mL) and H₂O (6 mL) was added cesium fluoride (0.718 g, 4.732 mmol, 2.0 equiv) and Pd(amphos)Cl₂ (0.168 g, 0.236 mmol, 0.1 equiv) at room temperature. The reaction mixture was purged with N₂ for 10 min and then heated at 90° C. for 6 h. Upon completion of reaction, as confirmed by LCMS, the reaction mixture was filtered through a Celite pad and washed with ethyl acetate (50 mL). The filtrate was diluted with water (50 mL) and extracted with ethyl acetate (2×50 mL). The combined organic layer was dried over Na₂SO₄ and concentrated under vacuum provide a crude which was purified by prep-HPLC (Column: SUNFIRE C18 250×30 mm, Method: TFA: ACN, Rₜ: 12 min, Flow rate: 15 mL/min) to provide compound 10 (0.6 g, 37.2%) as a yellow solid.

### Step-6: Synthesis of (R)—N-(5-chloro-2-fluoro-3-(1-(5-(piperazin-1-yl)pyridin-2-yl)-3-(pyridin-4-yl)-1H-pyrazol-4-yl)phenyl)-3-fluoropyrrolidine-1-sulfonamide (11)

To a solution of compound 10 (0.6 g, 0.856 mmol, 1.0 equiv) in DCM (20 mL) was added trifluoroacetic acid (0.976 g, 8.56 mmol, 10 equiv) at 0° C. and the reaction was warmed and stirred at room temperature for 2 h. The progress of the reaction was monitored by TLC (100% ethyl acetate, R_{f}: ~0.2). The reaction mixture was then concentrated under vacuum to give a brown gum, which was washed with petroleum ether (3×20 mL), dried under vacuum, and lyophilized to provide 11 (537 mg, 98%, TFA salt) as a yellow solid. LCMS: Rₜ: 1.261 min, Area: 96.8%, m/z=601.1 (M+H)⁺. Column: X-BRIDGE C8 (50×4.6 mm) 3.5 m; Mobile phase: A: 0.1% TFA in H₂O, B: ACN; Flow Rate: 2.0 mL/min. HPLC: Rₜ: 7.444 min, Area: 95.07%; Method Info: Column: Atlantis T3 150×4.6 mm, 3 μm; Mobile phase: A:0.1% TFA in water; Mobile phase: B: ACN; Flow: 1.2 mL/min. ¹H NMR (400 MHz, DMSO-d₆): 10.10 (s, 1H), 8.85 (s, 3H), 8.71 (d, J=6.40 Hz, 2H), 8.29 (d, J=2.80 Hz, 1H), 7.99 (d, J=9.20 Hz, 1H), 7.75-7.68 (m, 3H), 7.54 (q, J=2.40 Hz, 1H), 7.43 (q, J=2.40 Hz, 1H), 5.30 (d, J=52.00 Hz, 2H), 3.51 (q, J=8.00 Hz, 5H), 3.41-3.37 (m, 2H), 3.28 (t, J=2.40 Hz, 4H), 2.17-1.99 (m, 2H). ¹⁹F NMR (400 MHz, DMSO-d₆): −74.32, −126.08, −174.86.

### Synthesis of ethylmethyl[(5-chloro-2-fluoro-3-{1-[5-(piperazin-1-yl)pyridin-2-yl]-3-(pyridin-4-yl)-1H-pyrazol-4-yl}phenyl)sulfamoyl]amine (7)

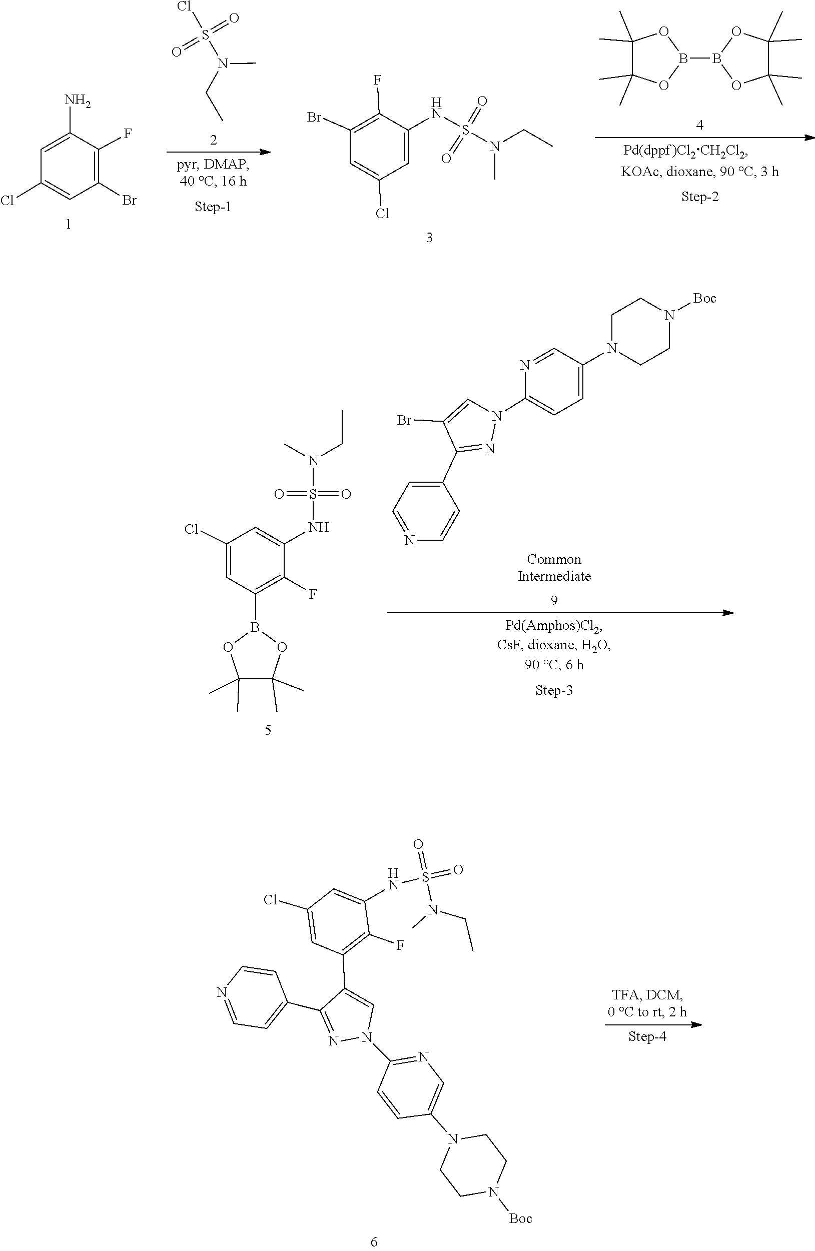

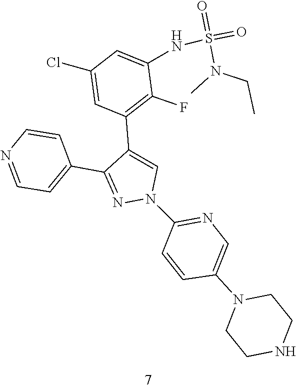 [SMILES: CCN(C)S(=O)(=O)Nc1cc(Cl)cc(-c2cn(-c3ccc(N4CCNCC4)cn3)nc2-c2ccncc2)c1F]

### Step-1: Synthesis of ethylmethyl[(3-bromo-5-chloro-2-fluorophenyl)sulfamoyl]amine (3)

To a solution of compound 1 (1 g, 4.46 mmol, 1.0 equiv) in pyridine (1.5 mL) was added DMAP (0.109 g, 0.891 mmol, 0.2 equiv) followed by compound 2 (0.913 g, 17.37 mmol, 1.3 equiv) at 0° C. and the resulting reaction mixture was heated to 40° C. for 16 h. Upon completion of the reaction, as confirmed by LCMS, the reaction mixture was concentrated under vacuum to afford a crude compound (1.5 g) as a brown gum. The crude compound was purified via Isolera (silica gel: 230-400 mesh) using ethyl acetate:petroleum ether (7-10%) as eluent to afford compound 3 (0.85 g, 49.5%) as an off-white solid.

### Step-2: Synthesis of ethylmethyl{[5-chloro-2-fluoro-3-(4,4,5,5-tetramethyl-1,3,2-dioxaborolan-2-yl)phenyl]sulfamoyl}amine (5)

To a solution of compound 3 (0.5 g, 1.447 mmol, 1.0 equiv) in 1,4-dioxane (10 mL) was added compound 4 (0.551 g, 2.17 mmol, 1.5 equiv) and potassium acetate (0.284 g, 2.89 mmol, 2.0 equiv) at room temperature. The resulting mixture was purged with N₂ for 15 min. Then PdCl₂(dppf) DCM (0.117 g, 0.145 mmol, 0.1 equiv) was added and the reaction was heated to 90° C. for 3 h. The progress of the reaction was monitored by TLC (50% ethyl acetate:petroleum ether, R_{f}: ~0.2). The reaction mixture was then cooled to room temperature and filtered through a Celite pad and washed with ethyl acetate (50 mL). The combined organic layer was passed through silica gel (100-120 mesh), dried over Na₂SO₄, and concentrated under vacuum to afford crude compound 5 (1.0 g, quant.) as a brown solid.

### Step-3: Synthesis of tert-butyl 4-(6-(4-(5-chloro-3-((N-ethyl-N-methylsulfamoyl)amino)-2-fluorophenyl)-3-(pyridin-4-yl)-1H-pyrazol-1-yl)pyridin-3-yl)piperazine-1-carboxylate (6)

To a mixture of compound 5 (2.0 g, 5.28 mmol, 3.0 equiv) and tert-butyl 4-(6-(4-bromo-3-(pyridin-4-yl)-1H-pyrazol-1-yl)pyridin-3-yl)piperazine-1-carboxylate (common intermediate 9, 0.85 g, 1.751 mmol, 1.0 equiv) and in 1,4-dioxane (30 mL) and water (7.5 mL) was added CsF (0.532 g, 3.5 mmol, 2.0 equiv) and Pd(Amphos)Cl₂ (0.124 g, 0.175 mmol, 0.1 equiv) and the reaction mixture was purged with nitrogen for 10 min. The reaction was then heated at 90° C. for 6 h. Upon completion of reaction as confirmed by LCMS, the reaction mixture was filtered through a Celite pad and washed with ethyl acetate (100 mL). The filtrate was diluted with water (50 mL) and extracted with ethyl acetate (2×100 mL). The combined organic layer was dried over Na₂SO₄ and concentrated under vacuum to provide a crude which was purified by prep-HPLC (Column: SUNFIRE C18 250×30 mm, Method: TFA: ACN, Rₜ: 12 min, Flow rate: 15 mL/min) to provide compound 6 (0.6 g, 51%) as a yellow solid.

### Step-4: Synthesis of ethylmethyl[(5-chloro-2-fluoro-3-{1-[5-(piperazin-1-yl)pyridin-2-yl]-3-(pyridin-4-yl)-1H-pyrazol-4-yl}phenyl)sulfamoyl]amine (7)

To a solution of compound 6 (0.6 g, 0.8939 mmol, 1.0 equiv) in DCM (14 mL) was added trifluoroacetic acid (0.70 mL, 8.939 mmol, 10 equiv) at 0° C. and the reaction was warmed and stirred at room temperature for 2 h. The progress of the reaction was monitored by TLC (100% ethyl acetate, R_{f}: ~0.2). The reaction mixture was concentrated under vacuum to provide a crude product as a brown gum. The crude was washed with petroleum ether (3×20 mL) and dried under vacuum to afford a crude compound (0.6 g, 92%) by LCMS. The crude was purified by prep-HPLC (Column: SUNFIRE C18 250×30 mm, Method: TFA: ACN, Rₜ: 12 min, Flow rate: 15 mL/min) and then lyophilized to provide 7 (392 mg, 98%, TFA salt) as a pale yellow solid. LCMS: Rₜ: 1.306 min, Area: 99.6%, m/z=571.2 (M+H)+; Column: X-BRIDGE C8 (50×4.6 mm) 3.5 m; Mobile phase: A: 0.1% TFA in H₂O, B: ACN; Flow Rate: 2.0 mL/min. HPLC: Rₜ: 7.527 min, Area: 99.31%; Method Info: Column: Atlantis T3 150×4.6 mm, 3 m; Mobile phase: A: 0.1% TFA in water; Mobile phase: B: ACN; Flow: 1.2 mL/min. ¹H NMR (400 MHz, DMSO-d₆): 9.96 (s, 1H), 8.87-8.85 (m, 3H), 8.66 (d, J=1.60 Hz, 2H), 8.28 (d, J=3.20 Hz, 1H), 7.98 (d, J=8.80 HZ, 1H), 7.73 (dd, J=3.20, 9.00 Hz, 1H), 7.58 (d, J=1.60 Hz, 2H), 7.47 (dd, J=2.80, 6.40 Hz, 1H), 7.41 (dd, J=2.80, 5.60 Hz, 1H), 3.32-3.50 (m, 4H), 3.30 (br s, 4H), 3.10 (q, J=7.20 Hz, 2H), 2.69 (s, 3H), 1.03 (t, J=7.20 Hz, 3H). ¹⁹F NMR (400 MHz, DMSO-d₆): −74.08, −126.55.

### Synthesis of N-(5-chloro-3-(1-(4-(3,3-dimethylpiperazin-1-yl)-2-fluorophenyl)-3-(pyridin-4-yl)-1H-pyrazol-4-yl)-2-fluorophenyl)pyrrolidine-1-sulfonamide TFA Salt (7)

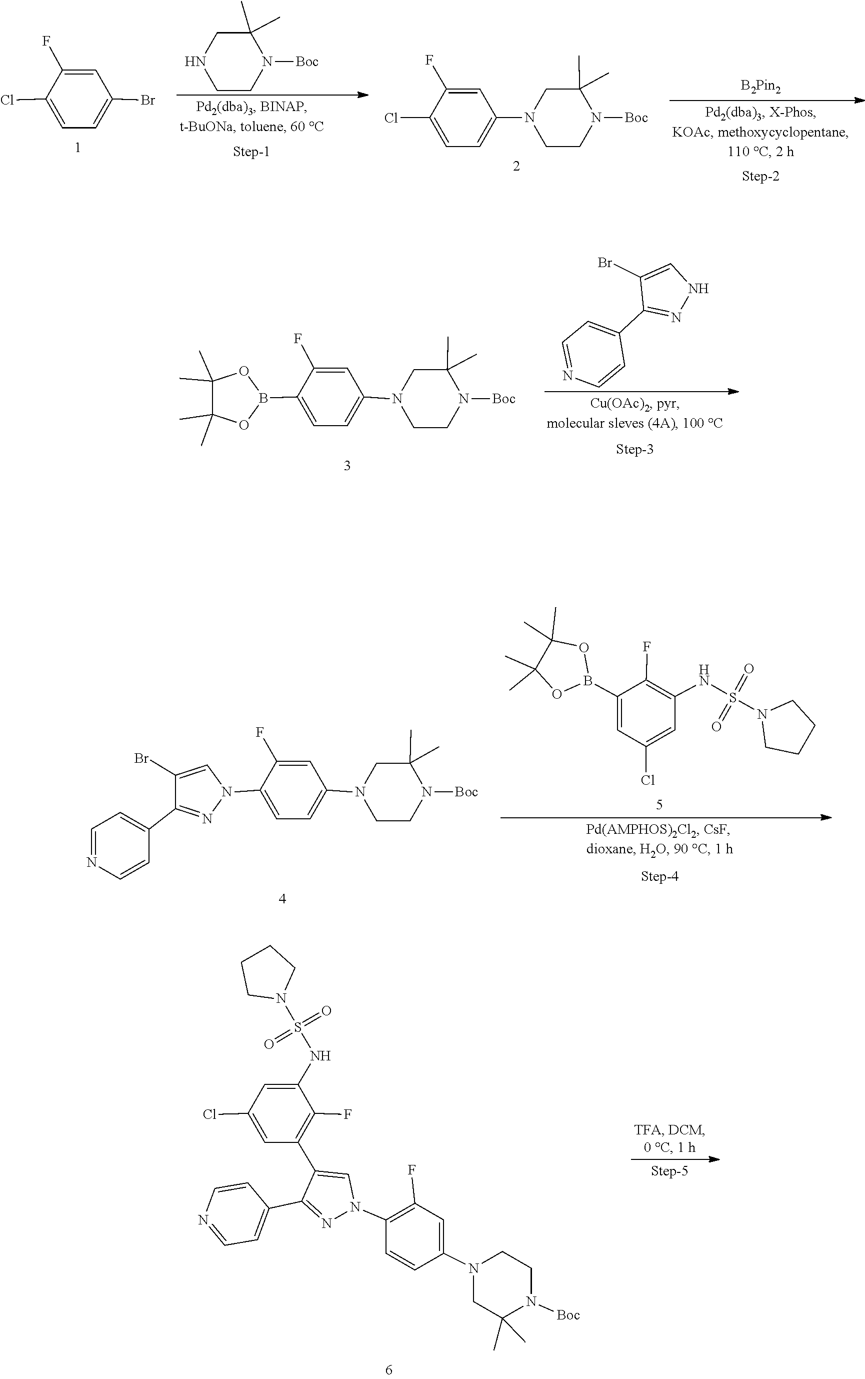

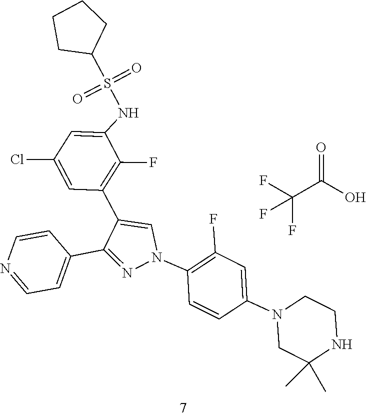 [SMILES: CC1(C)CN(c2ccc(-n3cc(-c4cc(Cl)cc(NS(=O)(=O)C5CCCC5)c4F)c(-c4ccncc4)n3)c(F)c2)CCN1 ; O=C(O)C(F)(F)F]

### Step-1: Synthesis of tert-butyl 4-(4-chloro-3-fluorophenyl)-2,2-dimethylpiperazine-1-carboxylate (2)

To a mixture of 4-bromo-1-chloro-2-fluorobenzene (3 g, 14.324 mmol, 1 equiv) and tert-butyl 2,2-dimethyl-1-piperazinecarboxylate (3.07 g, 14.324 mmol, 1 equiv) in toluene (60 mL) was added Pd₂(dba)₃ (131 mg, 0.143 mmol, 0.01 equiv), BINAP (178 mg, 0.286 mmol, 0.02 equiv), and t-BuONa (1.93 g, 20.054 mmol, 1.4 equiv). The resulting mixture was stirred at 60° C. overnight under a nitrogen atmosphere. The resulting mixture was then concentrated under reduced pressure. The residue was purified by silica gel column chromatography eluted with petroleum ether (PE):EtOAc (4:1) to afford tert-butyl 4-(4-chloro-3-fluorophenyl)-2,2-dimethylpiperazine-1-carboxylate (2.3 g, 46.84%) as an off-white solid. LCMS: C₁₇H₂₄ClFN₂O₂ requires: 342.2, found: m/z=343.3 [M+H]⁺.

### Step-2: Synthesis of tert-butyl 4-[3-fluoro-4-(4,4,5,5-tetramethyl-1,3,2-dioxaborolan-2-yl)phenyl]-2,2-dimethylpiperazine-1-carboxylate (3)

To a mixture of tert-butyl 4-(4-chloro-3-fluorophenyl)-2,2-dimethylpiperazine-1-carboxylate (2.1 g, 6.1253 mmol, 1 equiv) and 4,4,5,5-tetramethyl-2-(4,4,5,5-tetramethyl-1,3,2-dioxaborolan-2-yl)-1,3,2-dioxaborolane (B₂Pin₂) (2.38 g, 9.188 mmol, 1.5 equiv) in methoxycyclopentane (30 mL) was added Pd₂(dba)₃ (171 mg, 0.188 mmol, 0.03 equiv), X-Phos (176 mg, 0.376 mmol, 0.06 equiv), and KOAc (1.75 g, 18.375 mmol, 3 equiv). The resulting mixture was then stirred at 110° C. for 2.0 h under a nitrogen atmosphere. The resulting mixture was then concentrated under reduced pressure. The residue was purified by silica gel column chromatography eluted with PE:EtOAc (4:1) to afford tert-butyl 4-[3-fluoro-4-(4,4,5,5-tetramethyl-1,3,2-dioxaborolan-2-yl)phenyl]-2,2-dimethylpiperazine-1-carboxylate (1.3 g, 48.86%) as a brown oil. LCMS: C₂₃H₃₆BFN₂O₄ requires: 434.3, found: m/z=435.2 [M+H]⁺.

### Step-3: Synthesis of tert-butyl 4-{4-[4-bromo-3-(pyridin-4-yl)pyrazol-1-yl]-3-fluorophenyl}-2,2-dimethylpiperazine-1-carboxylate (4)

To a mixture of tert-butyl 4-[3-fluoro-4-(4,4,5,5-tetramethyl-1,3,2-dioxaborolan-2-yl)phenyl]-2,2-dimethylpiperazine-1-carboxylate (1.3 g, 2.993 mmol, 1 equiv) and 4-(4-bromo-1H-pyrazol-3-yl)pyridine (1.006 g, 4.489 mmol, 1.5 equiv) in pyridine (20 mL) was added Cu(OAc)₂ (1.09 g, 5.986 mmol, 2 equiv) and molecular sieves (4 A) (1.4 g). The resulting mixture was stirred at 100° C. overnight under an oxygen atmosphere. The resulting mixture was then concentrated under reduced pressure. The residue was purified by silica gel column chromatography eluted with CH₂Cl₂:MeOH (20:1) to afford tert-butyl 4-{4-[4-bromo-3-(pyridin-4-yl)pyrazol-1-yl]-3-fluorophenyl}-2,2-dimethylpiperazine-1-carboxylate (0.45 g, 28.35%) as a brown semi-solid. LCMS: C₂₅H₂₉BrFN₅O₂ requires: 529.1, found: m/z=530.2 [M+H]⁺.

### Step-4: Synthesis of tert-butyl 4-[4-(4-{5-chloro-2-fluoro-3-[(pyrrolidine-1-sulfonyl)amino]phenyl}-3-(pyridin-4-yl)pyrazol-1-yl)-3-fluorophenyl]-2,2-dimethylpiperazine-1-carboxylate (6)

To a mixture of tert-butyl 4-{4-[4-bromo-3-(pyridin-4-yl)pyrazol-1-yl]-3-fluorophenyl}-2,2-dimethylpiperazine-1-carboxylate (450 mg, 0.8483 mmol, 1 equiv) and N-[5-chloro-2-fluoro-3-(4,4,5,5-tetramethyl-1,3,2-dioxaborolan-2-yl)phenyl]pyrrolidine-1-sulfonamide (1.03 g, 2.545 mmol, 3 equiv) in dioxane (10 mL) was added CsF (258 mg, 1.70 mmol, 2 equiv) in H₂O (2 mL) and Pd(AMPHOS)₂Cl₂ (60 mg, 0.085 mmol, 0.1 equiv). The resulting mixture was then stirred at 90° C. for one hour under a nitrogen atmosphere. The resulting mixture was then concentrated under reduced pressure. The residue was purified by silica gel column chromatography eluted with PE:EtOAc (1:2) to afford tert-butyl 4-[4-(4-{5-chloro-2-fluoro-3-[(pyrrolidine-1-sulfonyl)amino]phenyl}-3-(pyridin-4-yl)pyrazol-1-yl)-3-fluorophenyl]-2,2-dimethylpiperazine-1-carboxylate (170 mg, 27.52%) as an off-white solid. LCMS: C₃₅H₄₀ClF₂N₇O₄S requires: 727.3, found: m/z=728.3 [M+H]⁺.

### Step-5: Synthesis of N-(5-chloro-3-(1-(4-(3,3-dimethylpiperazin-1-yl)-2-fluorophenyl)-3-(pyridin-4-yl)-1H-pyrazol-4-yl)-2-fluorophenyl)pyrrolidine-1-sulfonamide TFA Salt (7)

To a mixture of tert-butyl 4-[4-(4-{5-chloro-2-fluoro-3-[(pyrrolidine-1-sulfonyl)amino]phenyl}-3-(pyridin-4-yl)pyrazol-1-yl)-3-fluorophenyl]-2,2-dimethylpiperazine-1-carboxylate (170 mg, 0.2334 mmol, 1 equiv) in DCM (6 mL) was added TFA (3 mL) at 0° C. The resulting mixture was stirred at 0° C. for one hour. The resulting mixture was then concentrated under vacuum. The residue was purified by reverse phase flash chromatography eluted with ACN:H₂O (1:1) to afford N-(5-chloro-3-{1-[4-(3,3-dimethylpiperazin-1-yl)-2-fluorophenyl]-3-(pyridin-4-yl)pyrazol-4-yl}-2-fluorophenyl)pyrrolidine-1-sulfonamide trifluoroacetic acid (100.0 mg, 68.22%) as a yellow solid. LCMS: C₃₀H₃₂ClF₂N₇O₂S requires: 627.2, found: m/z=628.3 [M+H]⁺.

### Synthesis of N-(3-(1-(4-(3,3-dimethylpiperazin-1-yl)-2-fluorophenyl)-3-(pyridin-4-yl)-1H-pyrazol-4-yl)-2,5-difluorophenyl)pyrrolidine-1-sulfonamide TFA Salt

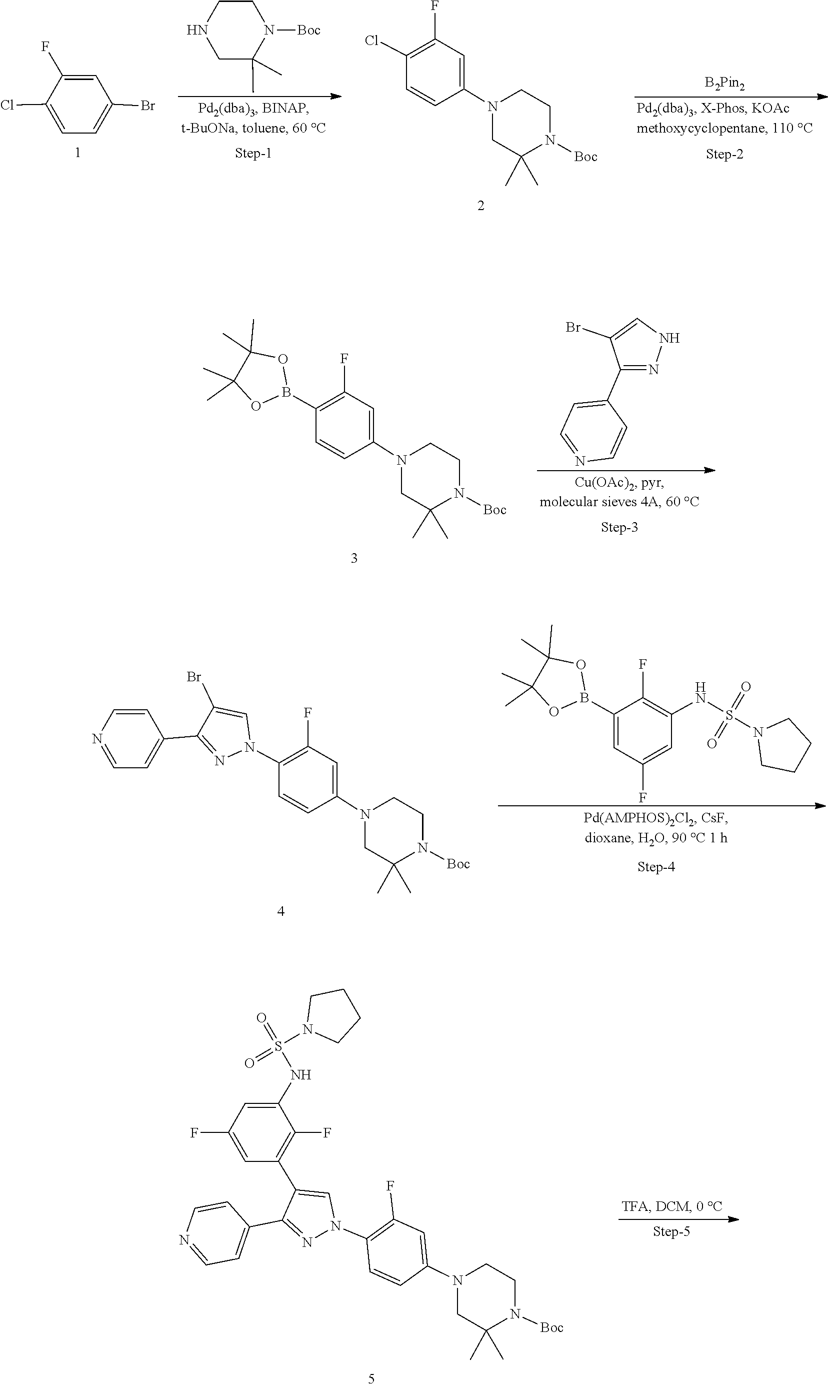

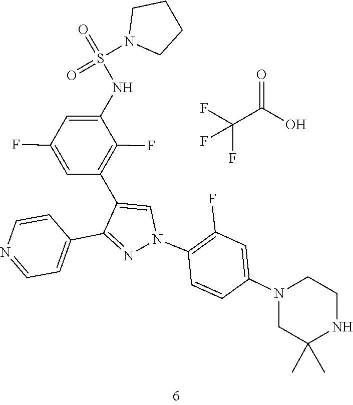

### Step-1: Synthesis of tert-butyl 4-(4-chloro-3-fluorophenyl)-2,2-dimethylpiperazine-1-carboxylate (2)

To a mixture of 4-bromo-1-chloro-2-fluorobenzene (5 g, 23.873 mmol, 1 equiv) and tert-butyl 2,2-dimethylpiperazine-1-carboxylate (5.12 g, 23.873 mmol, 1 equiv) in toluene (50 mL) was added Pd₂(dba)₃ (0.22 g, 0.239 mmol, 0.01 equiv), BINAP (0.30 g, 0.477 mmol, 0.02 equiv), and t-BuONa (3.21 g, 33.422 mmol, 1.4 equiv). The resulting mixture was stirred at 60° C. overnight under a nitrogen atmosphere. The resulting mixture was then concentrated under reduced pressure. The residue was purified by silica gel column chromatography eluted with PE:EtOAC (9:1) to afford tert-butyl 4-(4-chloro-3-fluorophenyl)-2,2-dimethylpiperazine-1-carboxylate (7.8 g, 95.30%) as an orange solid. LCMS: C₁₇H₂₄ClFN₂O₂ requires: 342.2, found: m/z=343.3 [M+H]⁺.

### Step-2: Synthesis of tert-butyl 4-[3-fluoro-4-(4,4,5,5-tetramethyl-1,3,2-dioxaborolan-2-yl)phenyl]-2,2-dimethylpiperazine-1-carboxylate (3)

To a mixture of tert-butyl 4-(4-chloro-3-fluorophenyl)-2,2-dimethylpiperazine-1-carboxylate (7.8 g, 22.751 mmol, 1 equiv) and bis(pinacolato)diboron (B₂Pin₂) (8.67 g, 34.127 mmol, 1.5 equiv) in methoxycyclopentane (80 mL) was added Pd₂(dba)₃ (0.63 g, 0.683 mmol, 0.03 equiv), XPhos (0.65 g, 1.365 mmol, 0.06 equiv), and AcOK (6.70 g, 68.253 mmol, 3 equiv). The resulting mixture was stirred at 110° C. overnight under a nitrogen atmosphere. The resulting mixture was then concentrated under reduced pressure. The residue was purified by silica gel column chromatography eluted with PE:EtOAc (9:1) to afford tert-butyl 4-[3-fluoro-4-(4,4,5,5-tetramethyl-1,3,2-dioxaborolan-2-yl)phenyl]-2,2-dimethylpiperazine-1-carboxylate (9 g, 91.07%) as an orange solid. LCMS: C₂₃H₃₆BFN₂O₄ requires: 434.3, found: m/z=435.2 [M+H]⁺.

### Step-3: Synthesis of tert-butyl 4-{4-[4-bromo-3-(pyridin-4-yl)pyrazol-1-yl]-3-fluorophenyl}-2,2-dimethylpiperazine-1-carboxylate (4)

To a mixture of tert-butyl 4-[3-fluoro-4-(4,4,5,5-tetramethyl-1,3,2-dioxaborolan-2-yl)phenyl]-2,2-dimethylpiperazine-1-carboxylate (2 g, 4.604 mmol, 1 equiv) and 4-(4-bromo-1H-pyrazol-3-yl)pyridine (1.13 g, 5.064 mmol, 1.1 equiv) in pyridine (20 mL) was added Cu(OAc)₂ (1.67 g, 9.208 mmol, 2 equiv) and molecular sieves (4 A) (2 g). The resulting mixture was stirred at 60° C. overnight under an oxygen atmosphere. The resulting mixture was then diluted with water and extracted with EtOAc. The combined organic layers were washed with water and dried over anhydrous Na₂SO₄. After filtration, the filtrate was concentrated under reduced pressure. The residue was purified by silica gel column chromatography eluted with PE:EtOAc (1:1) to afford tert-butyl 4-{4-[4-bromo-3-(pyridin-4-yl)pyrazol-1-yl]-3-fluorophenyl}-2,2-dimethylpiperazine-1-carboxylate (1 g, 40.94%) as a white solid. LCMS: C₂₅H₂₉BrFN₅O₂ requires: 529.2, found: m/z=530.1 [M+H]⁺.

### Step-4: Synthesis of tert-butyl 4-[4-(4-{2,5-difluoro-3-[(pyrrolidine-1-sulfonyl)amino]phenyl}-3-(pyridin-4-yl)pyrazol-1-yl)-3-fluorophenyl]-2,2-dimethylpiperazine-1-carboxylate (5)

To a mixture of N-[2,5-difluoro-3-(4,4,5,5-tetramethyl-1,3,2-dioxaborolan-2-yl)phenyl]pyrrolidine-1-sulfonamide (586 mg, 1.508 mmol, 1 equiv) and tert-butyl 4-(4-(4-bromo-3-(pyridin-4-yl)-1H-pyrazol-1-yl)-3-fluorophenyl)-2,2-dimethylpiperazine-1-carboxylate (800 mg, 1.508 mmol, 1 equiv) in dioxane (10 mL) was added Pd(AMPHOS)₂Cl₂ (107 mg, 0.151 mmol, 0.1 equiv) and CsF (458 mg, 3.016 mmol, 2 equiv) in H₂O (2 mL). The resulting mixture was stirred at 90° C. for one hour under a nitrogen atmosphere. The resulting mixture was then concentrated under reduced pressure. The residue was purified by silica gel column chromatography eluted with PE:EtOAc (1:1). The crude product was further purified by trituration with EtOAc to afford tert-butyl 4-[4-(4-{2,5-difluoro-3-[(pyrrolidine-1-sulfonyl)amino]phenyl}-3-(pyridin-4-yl)pyrazol-1-yl)-3-fluorophenyl]-2,2-dimethylpiperazine-1-carboxylate (250 mg, 23.29%) as a white solid. LCMS: C₃₅H₄₀F₃N₇O₄S requires: 711.3, found: m/z=712.4 [M+H]⁺.

### Step-5: Synthesis of N-(3-{1-[4-(3,3-dimethylpiperazin-1-yl)-2-fluorophenyl]-3-(pyridin-4-yl)pyrazol-4-yl}-2,5-difluorophenyl)pyrrolidine-1-sulfonamide TFA Salt (6)

To a mixture of tert-butyl 4-[4-(4-{2,5-difluoro-3-[(pyrrolidine-1-sulfonyl)amino]phenyl}-3-(pyridin-4-yl)pyrazol-1-yl)-3-fluorophenyl]-2,2-dimethylpiperazine-1-carboxylate (250 mg, 0.351 mmol, 1 equiv) in DCM (9 mL) was added TFA (3 mL) at 0° C. The resulting mixture was stirred at room temperature for one hour. The resulting mixture was then concentrated under vacuum to afford N-(3-(1-(4-(3,3-dimethylpiperazin-1-yl)-2-fluorophenyl)-3-(pyridin-4-yl)-1H-pyrazol-4-yl)-2,5-difluorophenyl)pyrrolidine-1-sulfonamide trifluoroacetic acid salt (219 mg, crude) as a yellow solid. LCMS: C₃₀H₃₂F₃N₇O₂S requires: 611.2, found: m/z=612.4 [M+H]⁺. ¹H NMR (400 MHz, Methanol-d₄) δ 8.78-8.70 (m, 2H), 8.40-8.35 (m, 1H), 8.12-8.05 (m, 2H), 7.88-7.79 (m, 1H), 7.45-7.36 (m, 1H), 7.14-7.01 (m, 3H), 3.60-3.53 (m, 2H), 3.48-3.44 (m, 2H), 3.42 (s, 2H), 3.33-3.30 (m, 3H), 1.96-1.84 (m, 5H), 1.53 (s, 6H).

### Synthesis of N-(2,5-difluoro-3-(1-(2-fluoro-4-(piperazin-1-yl)phenyl)-3-(pyridin-4-yl)-1H-pyrazol-4-yl)phenyl)cyclopentanesulfonamide TFA Salt (5)

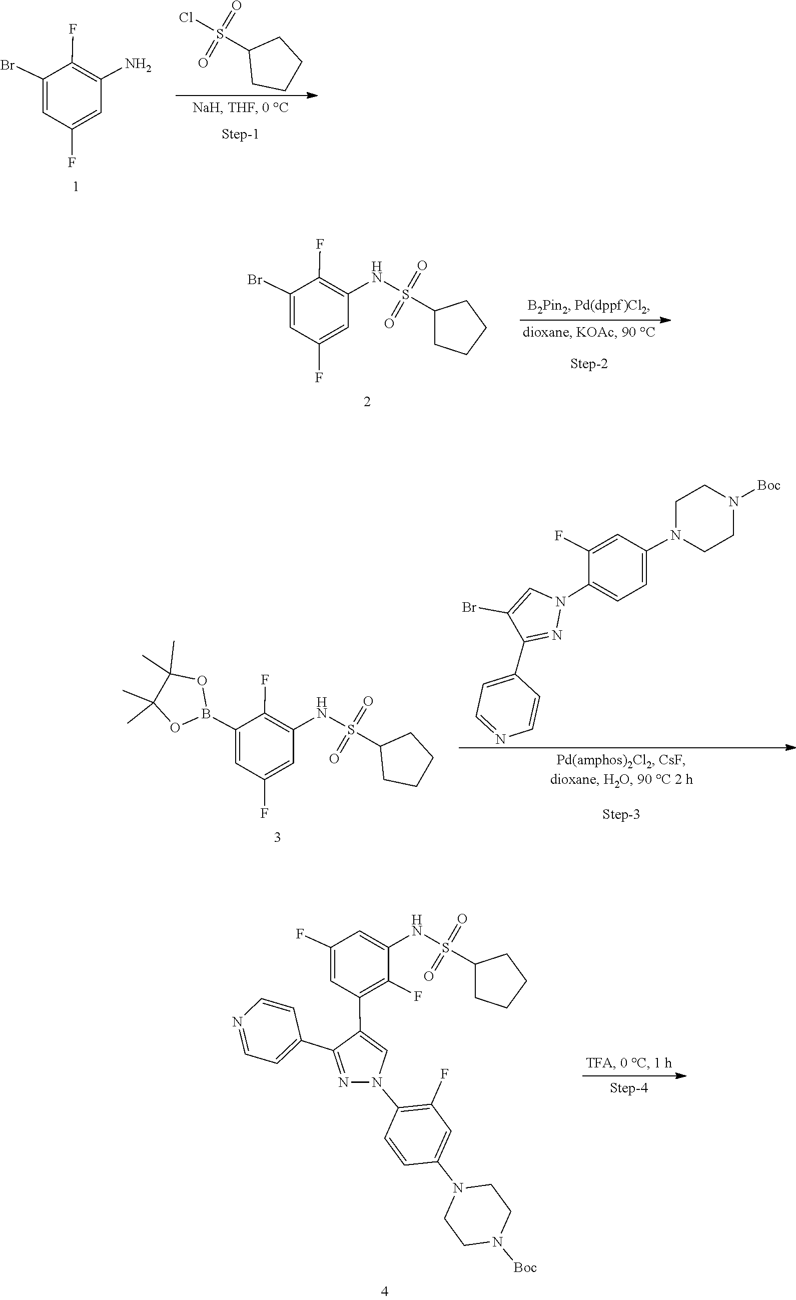 [SMILES: Nc1cc(F)cc(Br)c1F ; O=S(=O)(Cl)C1CCCC1 ; O=S(=O)(Nc1cc(F)cc(Br)c1F)C1CCCC1 ; CC1(C)OB(c2cc(F)cc(NS(=O)(=O)C3CCCC3)c2F)OC1(C)C ; CC(C)(C)OC(=O)N1CCN(c2ccc(-n3cc(Br)c(-c4ccncc4)n3)c(F)c2)CC1 ; CC(C)(C)OC(=O)N1CCN(c2ccc(-n3cc(-c4cc(F)cc(NS(=O)(=O)C5CCCC5)c4F)c(-c4ccncc4)n3)c(F)c2)CC1]

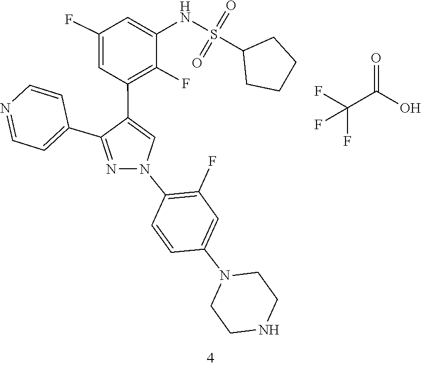

### Step-1: Synthesis of N-(3-bromo-2,5-difluorophenyl)cyclopentanesulfonamide (2)

To a mixture of NaH (1.08 g, 28.8454 mmol, 2 equiv, 60%) in THF (50 mL) was added 3-bromo-2,5-difluoroaniline (3 g, 14.4227 mmol, 1 equiv). The resulting mixture was stirred for one hour at 0° C. under a nitrogen atmosphere. To the above mixture was added cyclopentanesulfonyl chloride (4.86 g, 28.845 mmol, 2 equiv) at 0° C. The resulting mixture was then warmed and stirred at room temperature overnight under a nitrogen atmosphere. The reaction was then quenched by the addition of water at 0° C. The resulting mixture was concentrated under reduced pressure. The residue was purified by silica gel column chromatography eluted with PE:DCM (1:3) to afford N-(3-bromo-2,5-difluorophenyl)cyclopentanesulfonamide (639 mg, 13.02%) as an off-white solid. LCMS: C₁₁H₁₂BrF₂NO₂S requires: 340.0, found: m/z=341.1 [M−H]⁻.

### Step-2: Synthesis of N-[2,5-difluoro-3-(4,4,5,5-tetramethyl-1,3,2-dioxaborolan-2-yl)phenyl]cyclopentanesulfonamide (3)

To a mixture of N-(3-bromo-2,5-difluorophenyl)cyclopentanesulfonamide (639 mg, 1.878 mmol, 1 equiv) and 4,4,5,5-tetramethyl-2-(4,4,5,5-tetramethyl-1,3,2-dioxaborolan-2-yl)-1,3,2-dioxaborolane (B₂Pin₂) (953 mg, 3.754 mmol, 2 equiv) in dioxane (10 mL) was added KOAc (369 mg, 3.754 mmol, 2 equiv) and Pd(dppf)Cl₂ (137 mg, 0.188 mmol, 0.1 equiv). The resulting mixture was stirred at 90° C. overnight under a nitrogen atmosphere. The resulting mixture was then filtered. The filtrate was concentrated under reduced pressure. This resulted in N-[2,5-difluoro-3-(4,4,5,5-tetramethyl-1,3,2-dioxaborolan-2-yl)phenyl]cyclopentanesulfonamide (620 mg, crude) as a black oil. LCMS: C₁₇H₂₄BF₂NO₄S requires: 387.1, found: m/z=388.2 [M+H]⁺.

### Step-3: Synthesis of tert-butyl 4-{4-[4-(3-cyclopentanesulfonamido-2,5-difluorophenyl)-3-(pyridin-4-yl)pyrazol-1-yl]-3-fluorophenyl}piperazine-1-carboxylate

To a mixture of tert-butyl 4-{4-[4-bromo-3-(pyridin-4-yl)pyrazol-1-yl]-3-fluorophenyl}piperazine-1-carboxylate (700 mg, 1.3933 mmol, 1 equiv) and N-[2,5-difluoro-3-(4,4,5,5-tetramethyl-1,3,2-dioxaborolan-2-yl)phenyl]cyclopentanesulfonamide (540 mg, 1.3933 mmol, 1 equiv) in dioxane (10 mL) was added Pd(AMPHOS)₂Cl₂ (98 mg, 0.139 mmol, 0.1 equiv) and CsF (424 mg, 2.79 mmol, 2 equiv) in H₂O (2 mL). The resulting mixture was stirred at 90° C. for 2 h under a nitrogen atmosphere. The resulting mixture was then concentrated under reduced pressure. The residue was purified by silica gel column chromatography eluted with CH₂Cl₂:EtOAc (1:1) to afford tert-butyl 4-{4-[4-(3-cyclopentanesulfonamido-2,5-difluorophenyl)-3-(pyridin-4-yl)pyrazol-1-yl]-3-fluorophenyl}piperazine-1-carboxylate (192 mg, 20.29%) as an off-white solid. LCMS: C₃₄H₃₇F₃N₆O₄S requires: 682.3, found: m/z=683.1 [M+H]⁺.

### Step-4: Synthesis of N-(2,5-difluoro-3-{1-[2-fluoro-4-(piperazin-1-yl)phenyl]-3-(pyridin-4-yl)pyrazol-4-yl}phenyl)cyclopentanesulfonamide Trifluoroacetic Acid Salt (5)

A mixture of tert-butyl 4-{4-[4-(3-cyclopentanesulfonamido-2,5-difluorophenyl)-3-(pyridin-4-yl)pyrazol-1-yl]-3-fluorophenyl}piperazine-1-carboxylate (192 mg, 0.281 mmol, 1 equiv) in TFA (5 mL) was stirred at 0° C. for one hour. The resulting mixture was concentrated under reduced pressure and then lyophilized. This resulted in N-(2,5-difluoro-3-{1-[2-fluoro-4-(piperazin-1-yl)phenyl]-3-(pyridin-4-yl)pyrazol-4-yl}phenyl)cyclopentanesulfonamide trifluoroacetic acid slat (161 mg, crude) as a yellow solid. LCMS: C₂₉H₂₉F₃N₆O₂S requires: 582.2, found: m/z=583.3 [M+H]⁺.

### Synthesis of N-(2,5-difluoro-3-(1-(2-fluoro-4-(piperazin-1-yl)phenyl)-3-(pyridin-4-yl)-1H-pyrazol-4-yl)phenyl)cyclohexanesulfonamide TFA Salt (5)

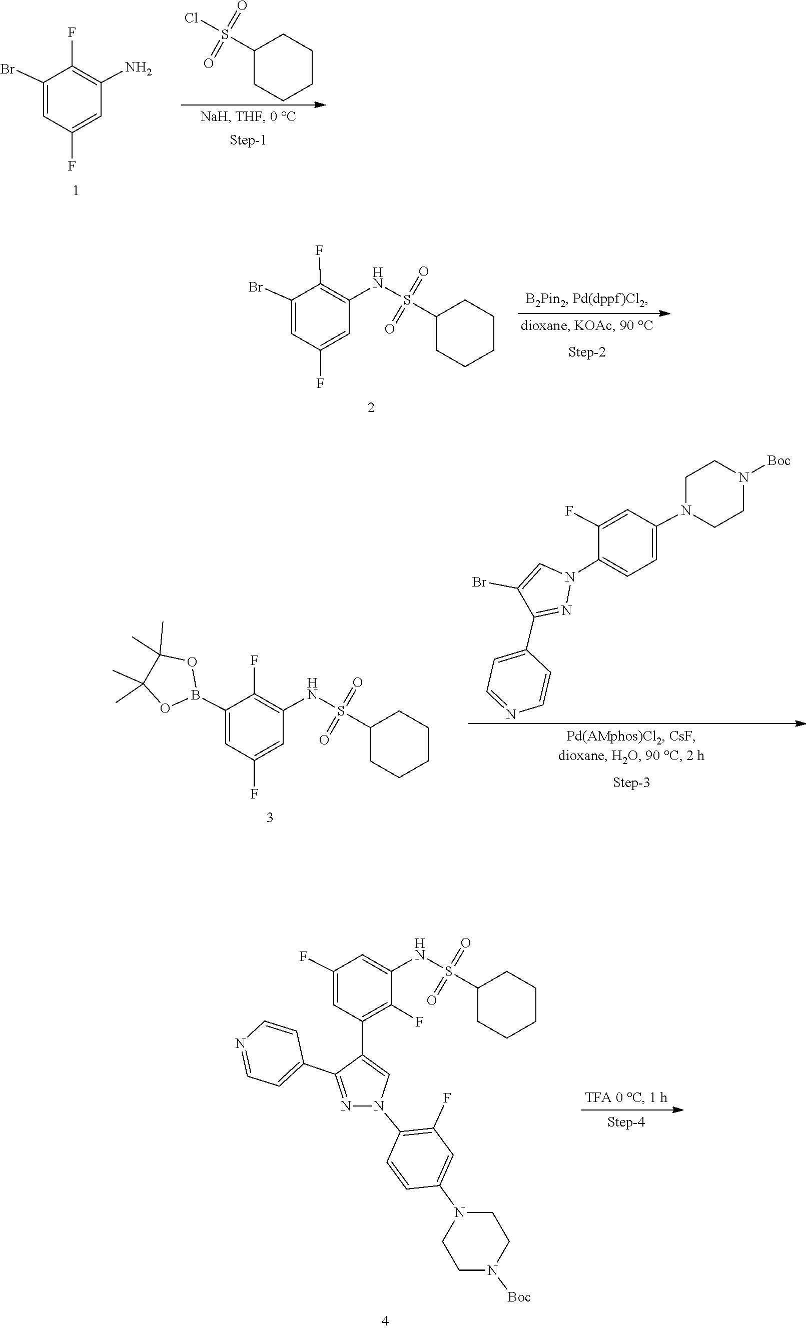 [SMILES: Nc1cc(F)cc(Br)c1F ; O=S(=O)(Cl)C1CCCCC1 ; O=S(=O)(Nc1cc(F)cc(Br)c1F)C1CCCCC1 ; CC1(C)OB(c2cc(F)cc(NS(=O)(=O)C3CCCCC3)c2F)OC1(C)C ; CC(C)(C)OC(=O)N1CCN(c2ccc(-n3cc(Br)c(-c4ccncc4)n3)c(F)c2)CC1 ; CC(C)(C)OC(=O)N1CCN(c2ccc(-n3cc(-c4cc(F)cc(NS(=O)(=O)C5CCCCC5)c4F)c(-c4ccncc4)n3)c(F)c2)CC1]

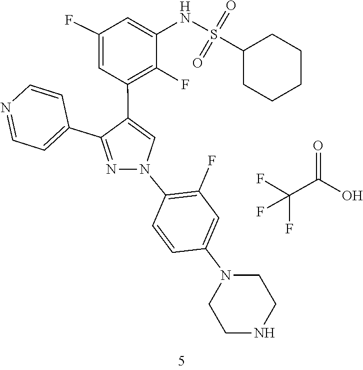

### Step-1: Synthesis of N-(3-bromo-2,5-difluorophenyl)cyclohexanesulfonamide (2)

To a mixture of NaH (1.26 g, 33.653 mmol, 2 equiv, 60%) in THF (60 mL) was added 3-bromo-2,5-difluoroaniline (3.5 g, 16.826 mmol, 1 equiv). The resulting mixture was stirred for one hour at 0° C. under a nitrogen atmosphere. To the above mixture was added cyclohexanesulfonyl chloride (6.18 g, 33.653 mmol, 2 equiv) at 0° C. The resulting mixture was stirred at room temperature overnight under a nitrogen atmosphere. The reaction was then quenched by the addition of water at 0° C. The resulting mixture was concentrated under reduced pressure. The residue was purified by silica gel column chromatography eluted with PE:DCM (1:3) to afford N-(3-bromo-2,5-difluorophenyl)cyclohexanesulfonamide (1.02 g, 17.1%) as an off-white solid. LCMS: C₁₂H₁₄BrF₂NO₂S requires: 353.0, found: m/z=354.4 [M−H]⁻.

### Step-2: Synthesis of N-[2,5-difluoro-3-(4,4,5,5-tetramethyl-1,3,2-dioxaborolan-2-yl)phenyl]cyclohexanesulfonamide (3)

To a mixture of N-(3-bromo-2,5-difluorophenyl)cyclohexanesulfonamide (800 mg, 1.878 mmol, 1 equiv) and 4,4,5,5-tetramethyl-2-(4,4,5,5-tetramethyl-1,3,2-dioxaborolan-2-yl)-1,3,2-dioxaborolane (953 mg, 3.754 mmol, 2 equiv) in dioxane (10 mL) was added KOAc (369 mg, 3.754 mmol, 2 equiv) and Pd(dppf)Cl₂ (137 mg, 0.188 mmol, 0.1 equiv). The resulting mixture was stirred at 90° C. overnight under a nitrogen atmosphere. The resulting mixture was then filtered. The filtrate was concentrated under reduced pressure. This resulted in N-[2,5-difluoro-3-(4,4,5,5-tetramethyl-1,3,2-dioxaborolan-2-yl)phenyl]cyclohexanesulfonamide (650 mg, crude) as a black oil. LCMS: C₁₈H₂₆BF₂NO₄S requires: 387.1, found: m/z=388.2 [M+H]⁺.

### Step-3: Synthesis of tert-butyl 4-(4-(4-(3-(cyclohexanesulfonamido)-2,5-difluorophenyl)-3-(pyridin-4-yl)-1H-pyrazol-1-yl)-3-fluorophenyl)piperazine-1-carboxylate (4)

To a mixture of tert-butyl 4-{4-[4-bromo-3-(pyridin-4-yl)pyrazol-1-yl]-3-fluorophenyl}piperazine-1-carboxylate (751 mg, 1.495 mmol, 1 equiv) and N-[2,5-difluoro-3-(4,4,5,5-tetramethyl-1,3,2-dioxaborolan-2-yl)phenyl]cyclohexanesulfonamide (600 mg, 1.495 mmol, 1 equiv) in dioxane (10 mL) was added Pd(AMPHOS)₂Cl₂ (105 mg, 0.150 mmol, 0.1 equiv) and CsF (454 mg, 2.99 mmol, 2 equiv) in H₂O (2 mL). The resulting mixture was stirred at 90° C. for 2 h under a nitrogen atmosphere. The resulting mixture was then concentrated under reduced pressure. The residue was purified by silica gel column chromatography eluted with CH₂Cl₂:EtOAc (1:1) to afford tert-butyl 4-(4-(4-(3-(cyclohexanesulfonamido)-2,5-difluorophenyl)-3-(pyridin-4-yl)-1H-pyrazol-1-yl)-3-fluorophenyl)piperazine-1-carboxylate (188 mg, 18%) as an off-white solid. LCMS: C₃₅H₃₉F₃N₆O₄S requires: 696.3, found: m/z=697.5 [M+H]⁺.

### Step-4: Synthesis of N-(2,5-difluoro-3-{1-[2-fluoro-4-(piperazin-1-yl)phenyl]-3-(pyridin-4-yl)pyrazol-4-yl}phenyl)cyclohexanesulfonamide Trifluoroacetic Acid Salt (5)

A mixture of tert-butyl 4-(4-(4-(3-(cyclohexanesulfonamido)-2,5-difluorophenyl)-3-(pyridin-4-yl)-1H-pyrazol-1-yl)-3-fluorophenyl)piperazine-1-carboxylate (188 mg, 0.27 mmol, 1 equiv) in TFA (4 mL) was stirred at 0° C. for one hour. The resulting mixture was then concentrated under reduced pressure and then lyophilized. This resulted in N-(2,5-difluoro-3-{1-[2-fluoro-4-(piperazin-1-yl)phenyl]-3-(pyridin-4-yl)pyrazol-4-yl}phenyl)cyclohexanesulfonamide trifluoroacetic acid salt (177 mg, crude) as a yellow solid. LCMS: C₃₀H₃₁F₃N₆O₂S requires: 596.2, found: m/z=597.2 [M+H]⁺.

### Synthesis of N-(2,5-difluoro-3-(1-(3-fluoro-4-(piperazin-1-yl)phenyl)-3-(pyridin-4-yl)-1H-pyrazol-4-yl)phenyl)cyclopentanesulfonamide TFA Salt (5)

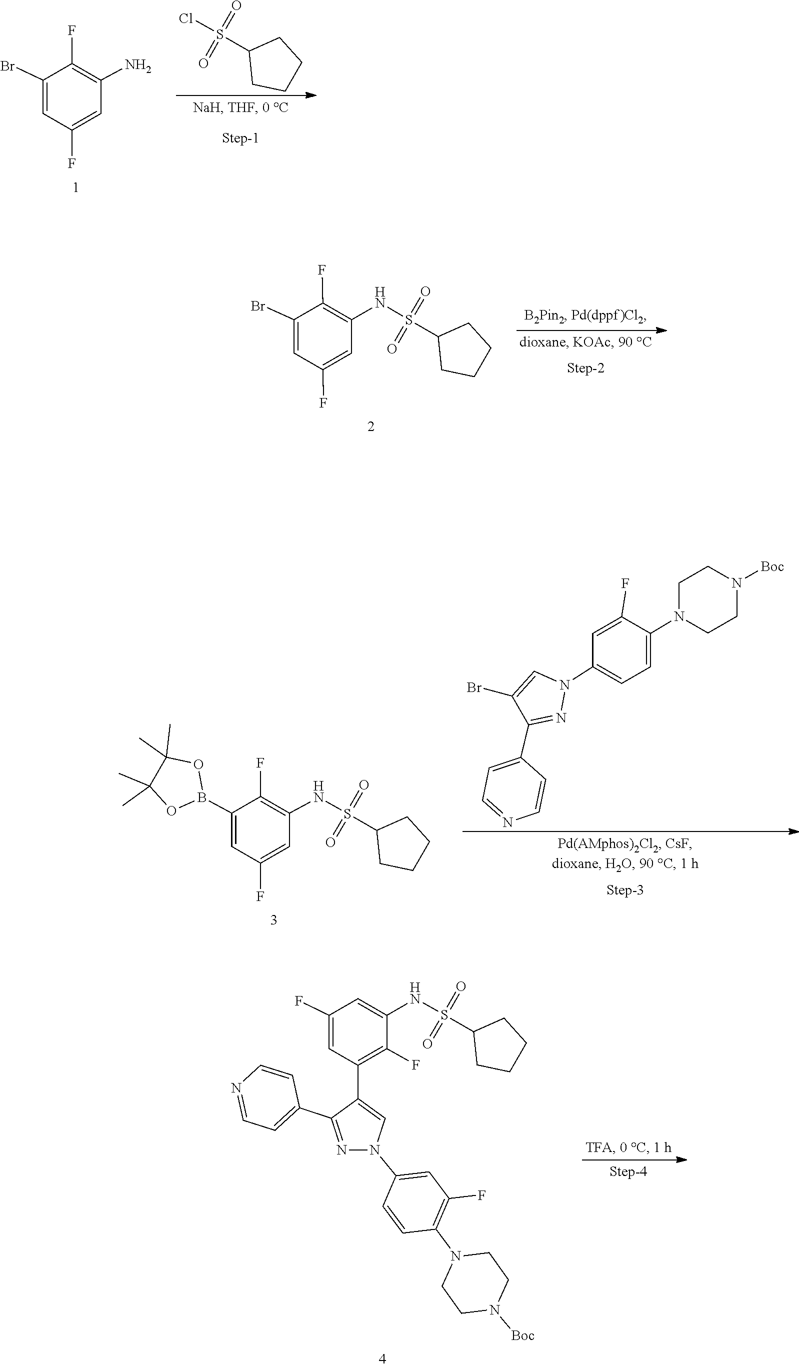 [SMILES: Nc1cc(F)cc(Br)c1F ; O=S(=O)(Cl)C1CCCC1 ; O=S(=O)(Nc1cc(F)cc(Br)c1F)C1CCCC1 ; CC1(C)OB(c2cc(F)cc(NS(=O)(=O)C3CCCC3)c2F)OC1(C)C ; CC(C)(C)OC(=O)N1CCN(c2ccc(-n3cc(Br)c(-c4ccncc4)n3)cc2F)CC1 ; CC(C)(C)OC(=O)N1CCN(c2ccc(-n3cc(-c4cc(F)cc(NS(=O)(=O)C5CCCC5)c4F)c(-c4ccncc4)n3)cc2F)CC1]

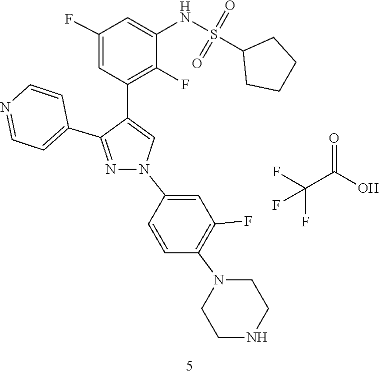

### Step-1: Synthesis of N-(3-bromo-2,5-difluorophenyl)cyclopentanesulfonamide (2)

To a mixture of NaH (1.08 g, 28.8454 mmol, 2 equiv, 60%) in THF (50 mL) was added 3-bromo-2,5-difluoroaniline (3 g, 14.4227 mmol, 1 equiv). The resulting mixture was stirred for one hour at 0° C. under a nitrogen atmosphere. To the above mixture was added cyclopentanesulfonyl chloride (4.86 g, 28.845 mmol, 2 equiv) at 0° C. The resulting mixture was stirred at room temperature overnight under a nitrogen atmosphere. The reaction was then quenched by the addition of water at 0° C. The resulting mixture was concentrated under reduced pressure. The residue was purified by silica gel column chromatography eluted with PE:DCM (1:3) to afford N-(3-bromo-2,5-difluorophenyl)cyclopentanesulfonamide (639 mg, 13.02%) as an off-white solid. LCMS: C₁₁H₁₂BrF₂NO₂S requires: 340.0, found: m/z=341.1 [M−H]⁻.

### Step-2: Synthesis of N-[2,5-difluoro-3-(4,4,5,5-tetramethyl-1,3,2-dioxaborolan-2-yl)phenyl]cyclopentanesulfonamide (3)

To a mixture of N-(3-bromo-2,5-difluorophenyl)cyclopentanesulfonamide (639 mg, 1.878 mmol, 1 equiv) and 4,4,5,5-tetramethyl-2-(4,4,5,5-tetramethyl-1,3,2-dioxaborolan-2-yl)-1,3,2-dioxaborolane (B₂Pin₂) (953 mg, 3.754 mmol, 2 equiv) in dioxane (10 mL) was added KOAc (369 mg, 3.754 mmol, 2 equiv) and Pd(dppf)Cl₂ (137 mg, 0.188 mmol, 0.1 equiv). The resulting mixture was stirred at 90° C. overnight under a nitrogen atmosphere. The resulting mixture was then filtered. The filtrate was concentrated under reduced pressure. This resulted in N-[2,5-difluoro-3-(4,4,5,5-tetramethyl-1,3,2-dioxaborolan-2-yl)phenyl]cyclopentanesulfonamide (620 mg, crude) as a black oil. LCMS: C₁₇H₂₄BF₂NO₄S requires: 387.1, found: m/z=388.2 [M+H]⁺.

### Step-3: Synthesis of tert-butyl 4-{4-[4-(3-cyclopentanesulfonamido-2,5-difluorophenyl)-3-(pyridin-4-yl)pyrazol-1-yl]-2-fluorophenyl}piperazine-1-carboxylate

To a mixture of tert-butyl 4-{4-[4-bromo-3-(pyridin-4-yl)pyrazol-1-yl]-2-fluorophenyl}piperazine-1-carboxylate (800 mg, 1.593 mmol, 1 equiv) and N-[2,5-difluoro-3-(4,4,5,5-tetramethyl-1,3,2-dioxaborolan-2-yl)phenyl]cyclopentanesulfonamide (617 mg, 1.5933 mmol, 1 equiv) in dioxane (10 mL) was added Pd(AMPHOS)₂Cl₂ (112 mg, 0.159 mmol, 0.1 equiv) and CsF (486 mg, 3.186 mmol, 2 equiv) in H₂O (2 mL). The resulting mixture was stirred at 90° C. for one hour under a nitrogen atmosphere. The resulting mixture was then concentrated under reduced pressure. The residue was purified by silica gel column chromatography eluted with CH₂Cl₂:EtOAc (1:1) to afford tert-butyl 4-{4-[4-(3-cyclopentanesulfonamido-2,5-difluorophenyl)-3-(pyridin-4-yl)pyrazol-1-yl]-2-fluorophenyl}piperazine-1-carboxylate (210 mg, 19.3%) as an off-white solid. LCMS: C₃₄H₃₇F₃N₆O₄S requires: 682.3, found: m/z=683.3 [M+H]⁺.

### Step-4: Synthesis of N-(2,5-difluoro-3-{1-[3-fluoro-4-(piperazin-1-yl)phenyl]-3-(pyridin-4-yl)pyrazol-4-yl}phenyl)cyclopentanesulfonamide Trifluoroacetic Acid Salt (5)

A mixture of tert-butyl 4-{4-[4-(3-cyclopentanesulfonamido-2,5-difluorophenyl)-3-(pyridin-4-yl)pyrazol-1-yl]-2-fluorophenyl}piperazine-1-carboxylate (210 mg, 0.307 mmol, 1 equiv) in TFA (5 mL) was stirred at 0° C. for one hour. The resulting mixture was then concentrated under reduced pressure and then lyophilized. This resulted in N-(2,5-difluoro-3-{1-[3-fluoro-4-(piperazin-1-yl)phenyl]-3-(pyridin-4-yl)pyrazol-4-yl}phenyl)cyclopentanesulfonamide trifluoroacetic acid salt (160 mg, crude) as a yellow solid. LCMS: C₂₉H₂₉F₃N₆O₂S requires: 582.2, found: m/z=583.3 [M+H]⁺.

### Synthesis of N-(5-chloro-2-fluoro-3-(1-(3-fluoro-4-(piperazin-1-yl)phenyl)-3-(pyridin-4-yl)-1H-pyrazol-4-yl)phenyl)cyclopentanesulfonamide TFA Salt (3)

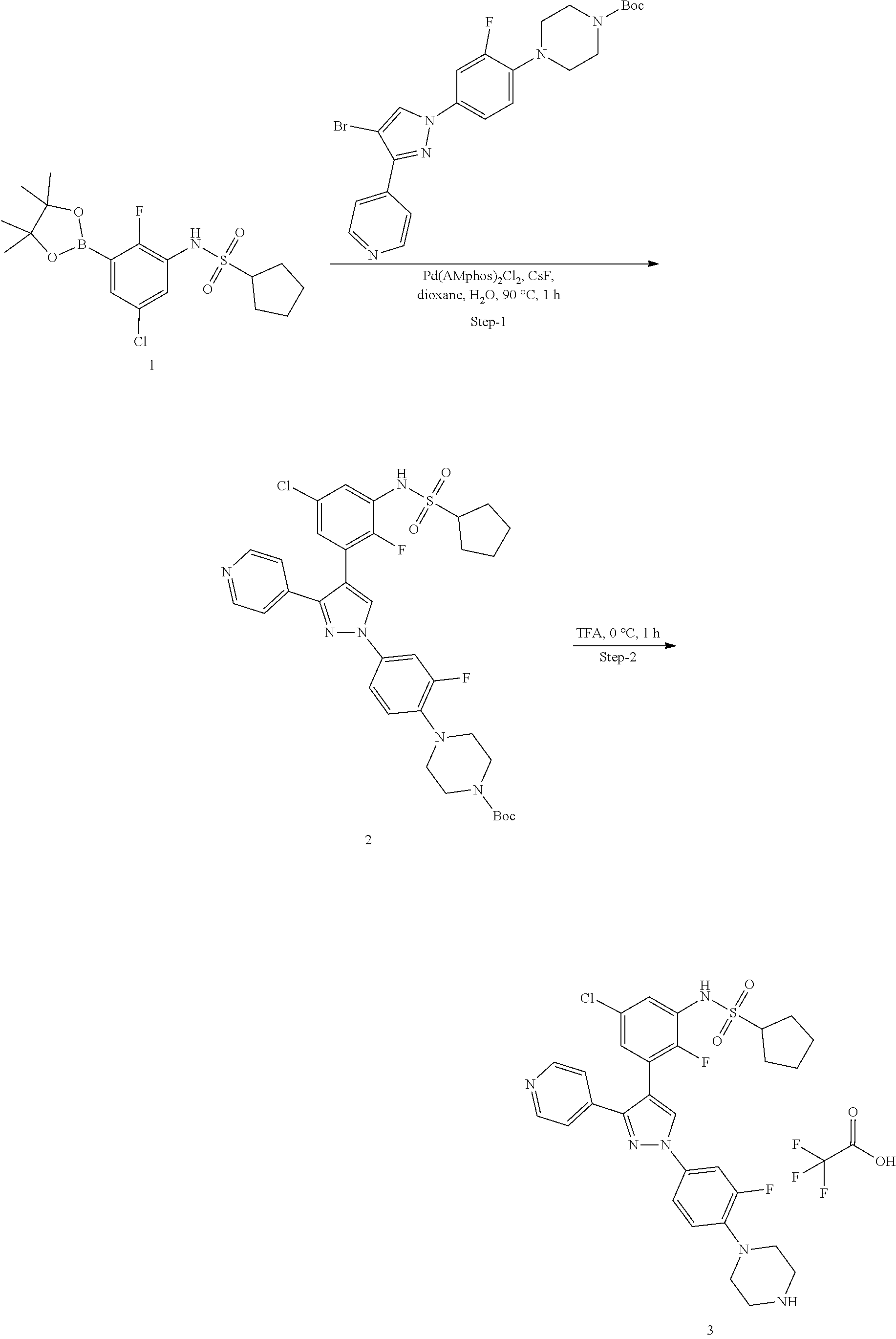

### Step-1: Synthesis of tert-butyl 4-{4-[4-(5-chloro-3-cyclopentanesulfonamido-2-fluorophenyl)-3-(pyridin-4-yl)pyrazol-1-yl]-2-fluorophenyl}piperazine-1-carboxylate (2)

To a mixture of tert-butyl 4-{4-[4-bromo-3-(pyridin-4-yl)pyrazol-1-yl]-2-fluorophenyl}piperazine-1-carboxylate (996 mg, 1.982 mmol, 1 equiv) and N-[5-chloro-2-fluoro-3-(4,4,5,5-tetramethyl-1,3,2-dioxaborolan-2-yl)phenyl]cyclopentanesulfonamide (800 mg, 1.982 mmol, 1 equiv) in dioxane (10 mL) was added Pd(AMPHOS)₂Cl₂ (140 mg, 0.198 mmol, 0.1 equiv) and CsF (606 mg, 3.964 mmol, 2 equiv) in H₂O (2 mL). The resulting mixture was stirred at 90° C. for one hour under a nitrogen atmosphere. The resulting mixture was then concentrated under reduced pressure. The residue was purified by silica gel column chromatography eluted with CH₂Cl₂:EtOAc (1:1) to afford tert-butyl 4-{4-[4-(5-chloro-3-cyclopentanesulfonamido-2-fluorophenyl)-3-(pyridin-4-yl)pyrazol-1-yl]-2-fluorophenyl}piperazine-1-carboxylate (350 mg, 25.3%) as an off-white solid. LCMS: C₃₄H₃₇ClF₂N₆O₄S requires: 698.2, found: m/z=699.3 [M+H]⁺.

### Step-2: Synthesis of N-(5-chloro-2-fluoro-3-{1-[3-fluoro-4-(piperazin-1-yl)phenyl]-3-(pyridin-4-yl)pyrazol-4-yl}phenyl)cyclopentanesulfonamide Trifluoroacetic Acid Salt (3)

A mixture of tert-butyl 4-{4-[4-(5-chloro-3-cyclopentanesulfonamido-2-fluorophenyl)-3-(pyridin-4-yl)pyrazol-1-yl]-2-fluorophenyl}piperazine-1-carboxylate (350 mg, 0.5 mmol, 1 equiv) in TFA (5 mL) was stirred at 0° C. for one hour. The resulting mixture was then concentrated under reduced pressure and then lyophilized. This resulted in N-(5-chloro-2-fluoro-3-{1-[3-fluoro-4-(piperazin-1-yl)phenyl]-3-(pyridin-4-yl)pyrazol-4-yl}phenyl)cyclopentanesulfonamide trifluoroacetic acid salt (276 mg, crude) as a yellow solid. LCMS: C₂₉H₂₉ClF₂N₆O₂S requires: 598.2, found: m/z=599.2 [M+H]⁺.

### Synthesis of (R)—N-(5-chloro-2-fluoro-3-(1-(2-fluoro-4-(3-methylpiperazin-1-yl)phenyl)-3-(pyridin-4-yl)-1H-pyrazol-4-yl)phenyl)cyclopentanesulfonamide TFA salt

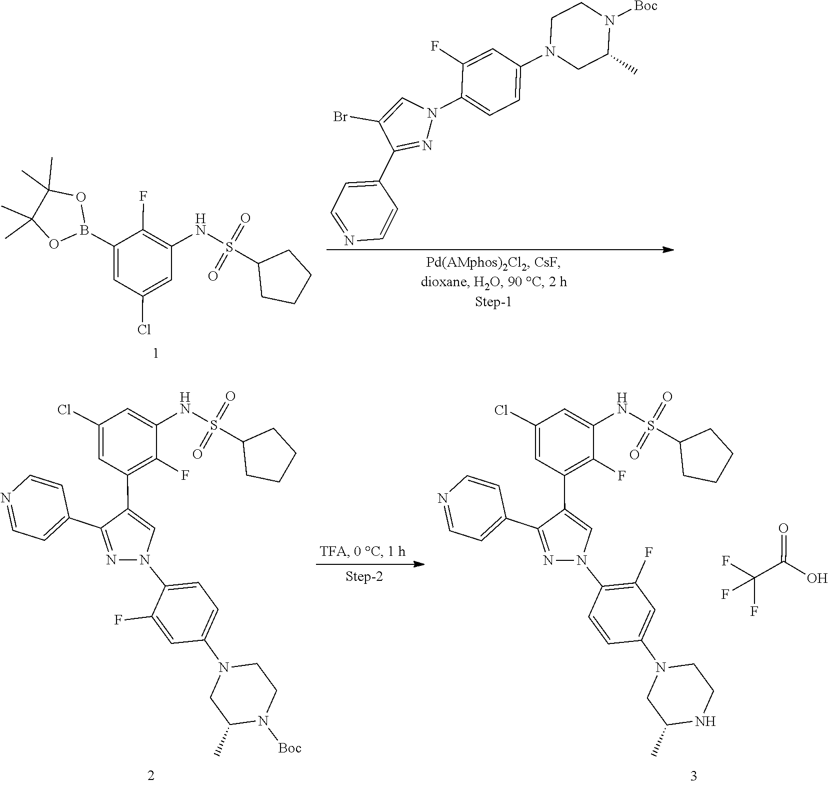 [SMILES: CC1(C)OB(c2cc(Cl)cc(NS(=O)(=O)C3CCCC3)c2F)OC1(C)C ; C[C@@H]1CN(c2ccc(-n3cc(Br)c(-c4ccncc4)n3)c(F)c2)CCN1C(=O)OC(C)(C)C ; C[C@@H]1CN(c2ccc(-n3cc(-c4cc(Cl)cc(NS(=O)(=O)C5CCCC5)c4F)c(-c4ccncc4)n3)c(F)c2)CCN1C(=O)OC(C)(C)C ; C[C@@H]1CN(c2ccc(-n3cc(-c4cc(Cl)cc(NS(=O)(=O)C5CCCC5)c4F)c(-c4ccncc4)n3)c(F)c2)CCN1 ; O=C(O)C(F)(F)F]

### Step-1: Synthesis of tert-butyl (2R)-4-{4-[4-(5-chloro-3-cyclopentanesulfonamido-2-fluorophenyl)-3-(pyridin-4-yl)pyrazol-1-yl]-3-fluorophenyl}-2-methylpiperazine-1-carboxylate (2)

To a mixture of tert-butyl (2R)-4-{4-[4-bromo-3-(pyridin-4-yl)pyrazol-1-yl]-3-fluorophenyl}-2-methylpiperazine-1-carboxylate (1.0 g, 1.936 mmol, 1 equiv) and N-[5-chloro-2-fluoro-3-(4,4,5,5-tetramethyl-1,3,2-dioxaborolan-2-yl)phenyl]cyclopentanesulfonamide (782 mg, 1.936 mmol, 1 equiv) in dioxane (10 mL) was added Pd(AMPHOS)₂Cl₂ (136 mg, 0.194 mmol, 0.1 equiv) and CsF (592 mg, 3.872 mmol, 2 equiv) in H₂O (2 mL). The resulting mixture was stirred at 90° C. for 2 h under a nitrogen atmosphere. The resulting mixture was then concentrated under reduced pressure. The residue was purified by silica gel column chromatography eluted with CH₂Cl₂:EtOAc (1:1) to afford tert-butyl (2R)-4-{4-[4-(5-chloro-3-cyclopentanesulfonamido-2-fluorophenyl)-3-(pyridin-4-yl)pyrazol-1-yl]-3-fluorophenyl}-2-methylpiperazine-1-carboxylate (400 mg, 28.96%) as an off-white solid. LCMS: C₃₅H₃₉ClF₂N₆O₄S requires: 712.2, found: m/z=713.4 [M+H]⁺.

### Step-2: Synthesis of N-[5-chloro-2-fluoro-3-(1-{2-fluoro-4-[(3R)-3-methylpiperazin-1-yl]phenyl}-3-(pyridin-4-yl)pyrazol-4-yl)phenyl]cyclopentanesulfonamide trifluoroacetic acid salt (3)

A mixture of tert-butyl (2R)-4-{4-[4-(5-chloro-3-cyclopentanesulfonamido-2-fluorophenyl)-3-(pyridin-4-yl)pyrazol-1-yl]-3-fluorophenyl}-2-methylpiperazine-1-carboxylate (400 mg, 0.561 mmol, 1 equiv) in TFA (6 mL) was stirred at 0° C. for one hour. The resulting mixture was then concentrated under reduced pressure and then lyophilized. This resulted N-[5-chloro-2-fluoro-3-(1-{2-fluoro-4-[(3R)-3-methylpiperazin-1-yl]phenyl}-3-(pyridin-4-yl)pyrazol-4-yl)phenyl]cyclopentanesulfonamide trifluoroacetic acid salt (321 mg, crude) as a yellow solid. LCMS: C₃₀H₃₁ClF₃N₆O₂S requires: 612.2, found: m/z=613.2 [M+H]⁺.

### Synthesis of (R)—N-(2,5-difluoro-3-(1-(2-fluoro-4-(3-methylpiperazin-1-yl)phenyl)-3-(pyridin-4-yl)-1H-pyrazol-4-yl)phenyl)cyclopentanesulfonamide TFA Salt

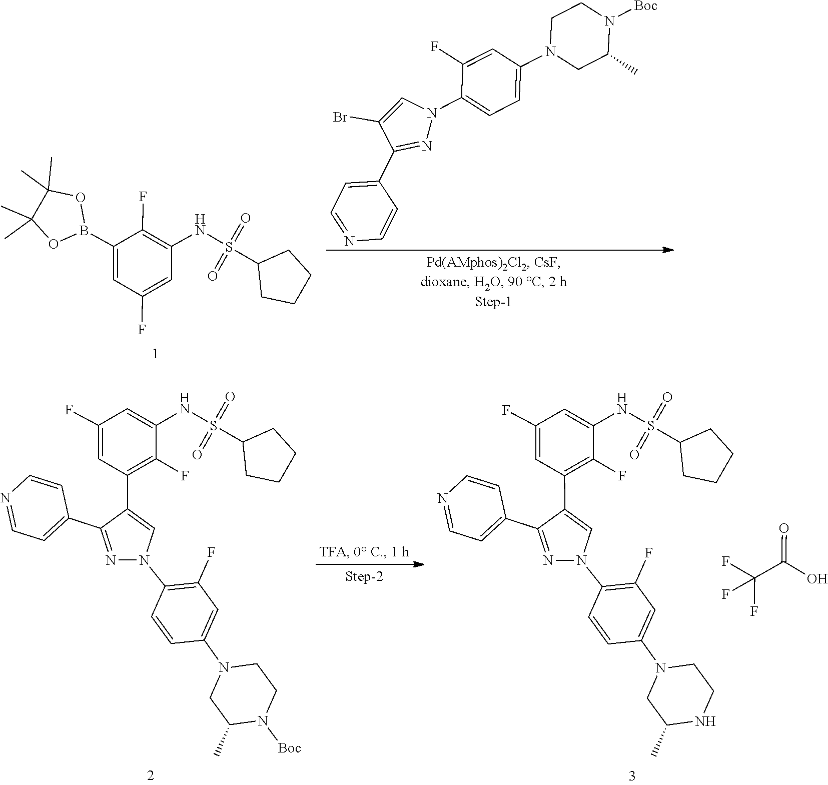 [SMILES: CC1(C)OB(c2cc(F)cc(NS(=O)(=O)C3CCCC3)c2F)OC1(C)C ; C[C@@H]1CN(c2ccc(-n3cc(Br)c(-c4ccncc4)n3)c(F)c2)CCN1C(=O)OC(C)(C)C ; C[C@@H]1CN(c2ccc(-n3cc(-c4cc(F)cc(NS(=O)(=O)C5CCCC5)c4F)c(-c4ccncc4)n3)c(F)c2)CCN1C(=O)OC(C)(C)C ; C[C@@H]1CN(c2ccc(-n3cc(-c4cc(F)cc(NS(=O)(=O)C5CCCC5)c4F)c(-c4ccncc4)n3)c(F)c2)CCN1 ; O=C(O)C(F)(F)F]

### Step-1: Synthesis of tert-butyl (2R)-4-{4-[4-(3-cyclopentanesulfonamido-2,5-difluorophenyl)-3-(pyridin-4-yl)pyrazol-1-yl]-3-fluorophenyl}-2-methylpiperazine-1-carboxylate (2)

To a mixture of tert-butyl (2R)-4-{4-[4-bromo-3-(pyridin-4-yl)pyrazol-1-yl]-3-fluorophenyl}-2-methylpiperazine-1-carboxylate (900 mg, 1.743 mmol, 1 equiv) and N-[2,5-difluoro-3-(4,4,5,5-tetramethyl-1,3,2-dioxaborolan-2-yl)phenyl]cyclopentanesulfonamide (675 mg, 1.743 mmol, 1 equiv) in dioxane (10 mL) was added Pd(AMPHOS)₂Cl₂ (122 mg, 0.174 mmol, 0.1 equiv) and CsF (533 mg, 3.486 mmol, 2 equiv) in H₂O (2 mL). The resulting mixture was stirred at 90° C. for 2 h under a nitrogen atmosphere. The resulting mixture was then concentrated under reduced pressure. The residue was purified by silica gel column chromatography eluted with CH₂Cl₂:EtOAc (1:1) to afford tert-butyl (2R)-4-{4-[4-(3-cyclopentanesulfonamido-2,5-difluorophenyl)-3-(pyridin-4-yl)pyrazol-1-yl]-3-fluorophenyl}-2-methylpiperazine-1-carboxylate (208 mg, 17.13%) as an off-white solid. LCMS: C₃₅H₃₉F₃N₆O₄S requires: 696.3, found: m/z=697.7 [M+H]⁺.

### Step-2: Synthesis of N-[2,5-difluoro-3-(1-{2-fluoro-4-[(3R)-3-methylpiperazin-1-yl]phenyl}-3-(pyridin-4-yl)pyrazol-4-yl)phenyl]cyclopentanesulfonamide; trifluoroacetic Acid Salt (3)

A mixture of tert-butyl (2R)-4-{4-[4-(3-cyclopentanesulfonamido-2,5-difluorophenyl)-3-(pyridin-4-yl)pyrazol-1-yl]-3-fluorophenyl}-2-methylpiperazine-1-carboxylate (208 mg, 0.2985 mmol, 1 equiv) in TFA (4 mL) was stirred at 0° C. for one hour. The resulting mixture was then concentrated under reduced pressure and then lyophilized. This resulted N-[2,5-difluoro-3-(1-{2-fluoro-4-[(3R)-3-methylpiperazin-1-yl]phenyl}-3-(pyridin-4-yl)pyrazol-4-yl)phenyl]cyclopentanesulfonamide trifluoroacetic acid salt (171 mg, crude) as a yellow solid. LCMS: C₃₀H₃₁F₄N₆O₂S requires: 596.2, found: m/z=597.4 [M+H]⁺.

### Synthesis of N-(3-(1-(2,6-difluoro-4-(piperazin-1-yl)phenyl)-3-(pyridin-4-yl)-1H-pyrazol-4-yl)-2,5-difluorophenyl)cyclopentanesulfonamide TFA Salt (7)

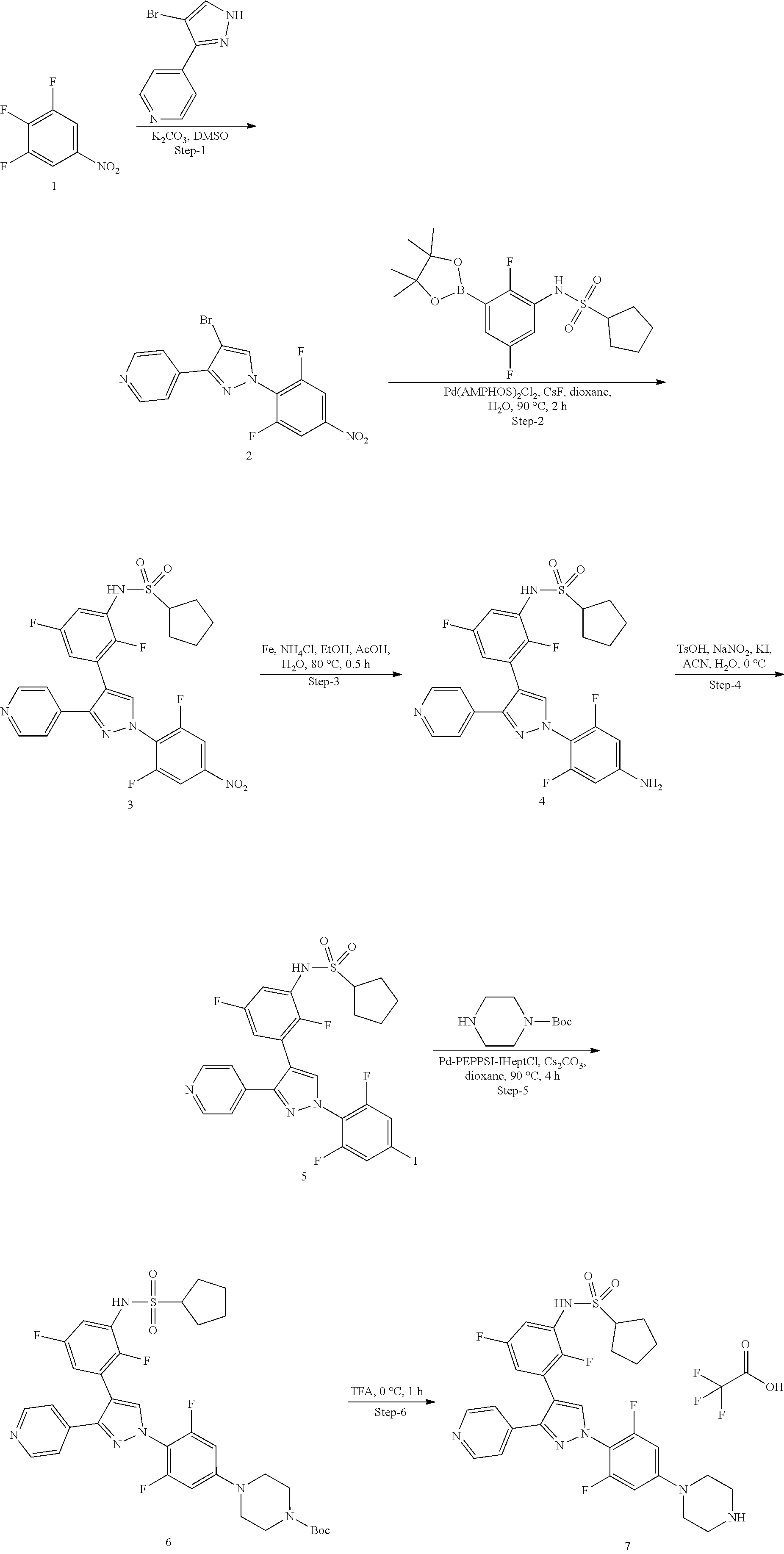

### Step-1: Synthesis of 4-[4-bromo-1-(2,6-difluoro-4-nitrophenyl)pyrazol-3-yl]pyridine (2)

To a mixture of 4-(4-bromo-1H-pyrazol-3-yl)pyridine (29 g, 129.43 mmol, 1 equiv) in DMSO (200 mL) was added K₂CO₃ (27 g, 194.1 mmol, 1.5 equiv) and 1,2,3-trifluoro-5-nitrobenzene (27.5 g, 155.32 mmol, 1.2 equiv). The resulting mixture was stirred at room temperature for 6 h. The resulting mixture was then extracted with CH₂Cl₂. The combined organic layers were washed with water and dried over anhydrous Na₂SO₄. After filtration, the filtrate was concentrated under reduced pressure. The residue was purified by silica gel column chromatography eluted with PE:EtOAc (3:1) to afford 4-[4-bromo-1-(2,6-difluoro-4-nitrophenyl)pyrazol-3-yl]pyridine (44 g, 89.2%) as a white solid. LCMS: C₁₄H₇BrF₂N₄O₂ requires: 379.9, found: m/z=380.9 [M+H]⁺.

### Step-2: Synthesis of N-{3-[1-(2,6-difluoro-4-nitrophenyl)-3-(pyridin-4-yl)pyrazol-4-yl]-2,5-difluorophenyl}cyclopentanesulfonamide (3)

To a mixture of 4-[4-bromo-1-(2,6-difluoro-4-nitrophenyl)pyrazol-3-yl]pyridine (9.0 g, 23.61 mmol, 1 equiv) and N-[5-chloro-2-fluoro-3-(4,4,5,5-tetramethyl-1,3,2-dioxaborolan-2-yl)phenyl]pyrrolidine-1-sulfonamide (9.1 g, 23.61 mmol, 1 equiv) in dioxane (100 mL) was added Pd(AMPHOS)₂Cl₂ (1.63 g, 2.361 mmol, 0.1 equiv) and CsF (7.15 g, 47.22 mmol, 2 equiv) in H₂O (20 mL). The resulting mixture was stirred at 90° C. for 2 h under a nitrogen atmosphere. The resulting mixture was then concentrated under vacuum. The residue was purified by silica gel column chromatography eluted with PE:EtOAc (1:1) to afford N-{3-[1-(2,6-difluoro-4-nitrophenyl)-3-(pyridin-4-yl)pyrazol-4-yl]-2,5-difluorophenyl}cyclopentanesulfonamide (5.3 g, 40%) as a yellow solid. LCMS: C₂₅H₁₉F₄N₅O₄S requires: 561.1, found: m/z=562.1 [M+H]⁺.

### Step-3: Synthesis of N-{3-[1-(4-amino-2,6-difluorophenyl)-3-(pyridin-4-yl)pyrazol-4-yl]-2,5-difluorophenyl}cyclopentanesulfonamide (4)

A mixture of Fe (2.64 g, 47.2 mmol, 5 equiv) and NH₄Cl (49 mg, 0.944 mmol, 0.1 equiv) in AcOH (2 mL) and H₂O (10 mL) was stirred at 80° C. for 5 min. To the above mixture was added N-{3-[1-(2,6-difluoro-4-nitrophenyl)-3-(pyridin-4-yl)pyrazol-4-yl]-2,5-difluorophenyl}cyclopentanesulfonamide (5.3 g, 9.439 mmol, 1 equiv) in EtOH (100 mL). The resulting mixture was stirred at 80° C. for 0.5 h. The resulting mixture was then concentrated under vacuum. The residue was purified by silica gel column chromatography eluted with PE:EtOAc (1:4) to afford N-{3-[1-(4-amino-2,6-difluorophenyl)-3-(pyridin-4-yl)pyrazol-4-yl]-2,5-difluorophenyl}cyclopentanesulfonamide (3.7 g, 73.7%) as a yellow solid. LCMS: C₂₅H₁₉F₄N₅O₂S requires: 531.1, found: m/z=532.2 [M+H]⁺.

### Step-4: Synthesis of N-{3-[1-(2,6-difluoro-4-iodophenyl)-3-(pyridin-4-yl)pyrazol-4-yl]-2,5-difluorophenyl}cyclopentanesulfonamide (5)

To a mixture of N-{3-[1-(4-amino-2,6-difluorophenyl)-3-(pyridin-4-yl)pyrazol-4-yl]-2,5-difluorophenyl}cyclopentanesulfonamide (3.7 g, 6.961 mmol, 1 equiv) and TsOH (3.64 g, 20.88 mmol, 3 equiv) in ACN (30 mL) was added NaNO₂ (1.46 g, 20.88 mmol, 3 equiv) in H₂O (3 mL) and KI (4.0 g, 17.40 mmol, 2.5 equiv) at 0° C. The resulting mixture was stirred at 0° C. for 10 min and was then warmed to room temperature. The mixture was stirred for additional 30 min at room temperature. The mixture was then basified to pH=8 with saturated aqueous Na₂CO₃. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with PE:EtOAc (3:2) to afford N-{3-[1-(2,6-difluoro-4-iodophenyl)-3-(pyridin-4-yl)pyrazol-4-yl]-2,5-difluorophenyl}cyclopentanesulfonamide (770 mg, 17.2%) as a brown solid. LCMS: C₂₅H₁₉F₄₁N₄O₂S requires: 642.0, found: m/z=643.7 [M+H]⁺.

### Step-5: Synthesis of tert-butyl 4-{4-[4-(3-cyclopentanesulfonamido-2,5-difluorophenyl)-3-(pyridin-4-yl)pyrazol-1-yl]-3,5-difluorophenyl}piperazine-1-carboxylate (6)

To a mixture of N-{3-[1-(2,6-difluoro-4-iodophenyl)-3-(pyridin-4-yl)pyrazol-4-yl]-2,5-difluorophenyl}cyclopentanesulfonamide (770 mg, 1.199 mmol, 1 equiv) and tert-butyl piperazine-1-carboxylate (558 mg, 2.998 mmol, 2.5 equiv) in dioxane (10 mL) was added Pd-PEPPSI-IHeptCl (116 mg, 0.12 mmol, 0.1 equiv) and Cs₂CO₃ (976 mg, 2.998 mmol, 2.5 equiv). The resulting mixture was stirred at 90° C. for 4 h under a nitrogen atmosphere. The resulting mixture was then concentrated under vacuum. The residue was purified by silica gel column chromatography eluted with PE:EtOAc (1:2) to afford tert-butyl 4-{4-[4-(3-cyclopentanesulfonamido-2,5-difluorophenyl)-3-(pyridin-4-yl)pyrazol-1-yl]-3,5-difluorophenyl}piperazine-1-carboxylate (320 mg, 38.1%) as a brown solid. LCMS: C₃₄H₃₆F₄N₆O₄S requires: 700.2, found: m/z=701.4 [M+H]⁺.

### Step-6: Synthesis of N-(3-{1-[2,6-difluoro-4-(piperazin-1-yl)phenyl]-3-(pyridin-4-yl)pyrazol-4-yl}-2,5-difluorophenyl)cyclopentanesulfonamide Trifluoroacetic Acid Salt (7)

A mixture of tert-butyl 4-{4-[4-(3-cyclopentanesulfonamido-2,5-difluorophenyl)-3-(pyridin-4-yl)pyrazol-1-yl]-3,5-difluorophenyl}piperazine-1-carboxylate (320 mg, 0.457 mmol, 1 equiv) in TFA (5 mL) was stirred at 0° C. for one hour. The resulting mixture was then concentrated under vacuum. The product was lyophilized to afford N-(3-{1-[2,6-difluoro-4-(piperazin-1-yl)phenyl]-3-(pyridin-4-yl)pyrazol-4-yl}-2,5-difluorophenyl)cyclopentanesulfonamide trifluoroacetic acid salt (210 mg, crude) as a light yellow solid. LCMS: C₂₉H₂₈F₄N₆O₄S requires: 600.2, found: m/z=601.2 [M+H]⁺.

### Synthesis of N-(5-chloro-3-(1-(2,6-difluoro-4-(piperazin-1-yl)phenyl)-3-(pyridin-4-yl)-1H-pyrazol-4-yl)-2-fluorophenyl)cyclopentanesulfonamide TFA Salt (7)

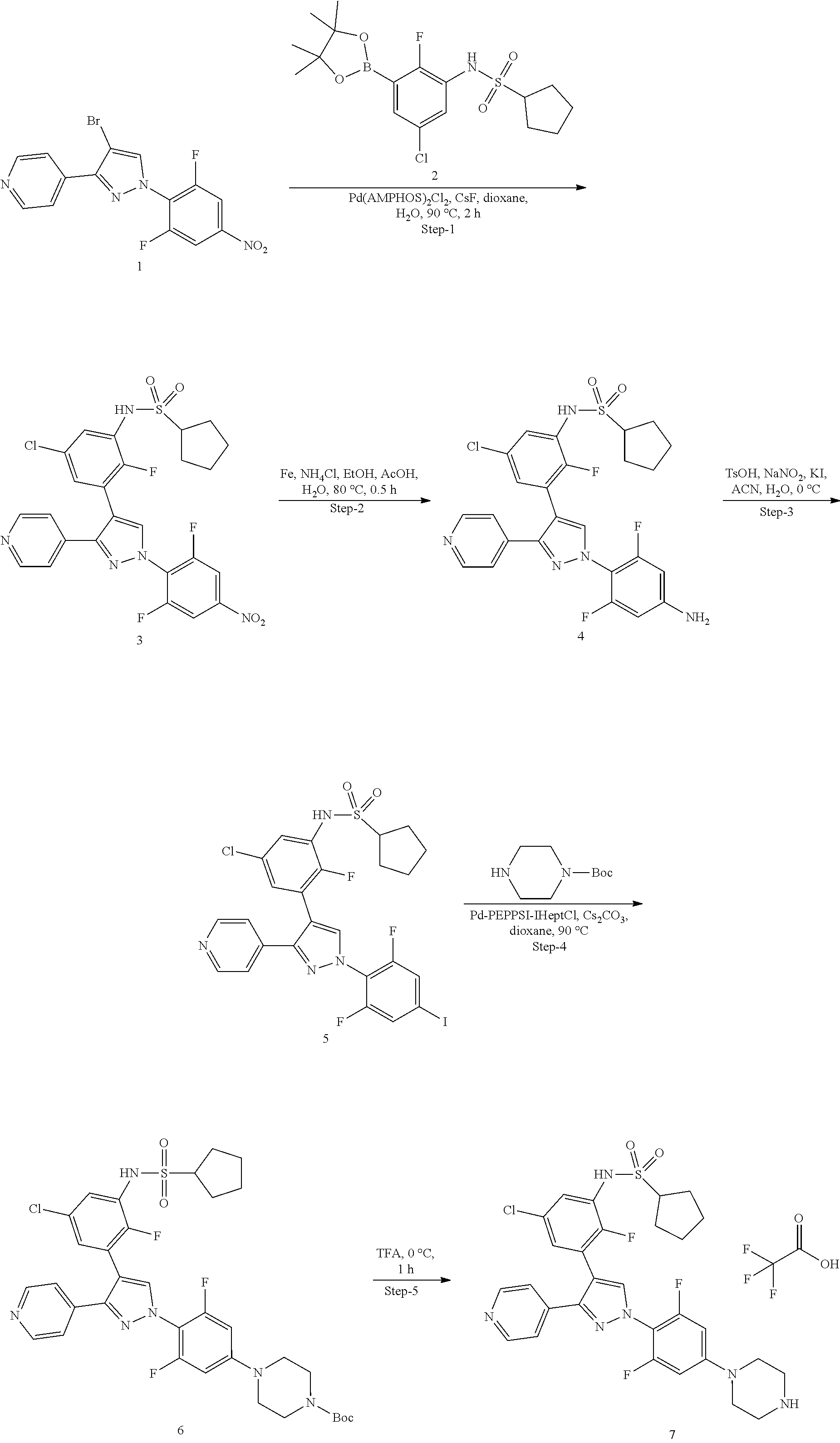

### Step-1: Synthesis of N-(5-chloro-3-(1-(2,6-difluoro-4-nitrophenyl)-3-(pyridin-4-yl)-1H-pyrazol-4-yl)-2-fluorophenyl)cyclopentanesulfonamide (3)

To a mixture of 4-[4-bromo-1-(2,6-difluoro-4-nitrophenyl)pyrazol-3-yl]pyridine (9.0 g, 23.61 mmol, 1 equiv) and N-[5-chloro-2-fluoro-3-(4,4,5,5-tetramethyl-1,3,2-dioxaborolan-2-yl)phenyl]cyclopentanesulfonamide (9.53 g, 23.61 mmol, 1 equiv) in dioxane (100 mL) was added Pd(AMPHOS)₂Cl₂ (1.64 g, 2.361 mmol, 0.1 equiv) and CsF (7.2 g, 47.22 mmol, 2 equiv) in H₂O (20 mL). The resulting mixture was stirred at 90° C. for 2 h under a nitrogen atmosphere. The resulting mixture was then concentrated under reduced pressure. The residue was purified by silica gel column chromatography eluted with PE:EtOAc (1:1) to afford N-(5-chloro-3-(1-(2,6-difluoro-4-nitrophenyl)-3-(pyridin-4-yl)-1H-pyrazol-4-yl)-2-fluorophenyl)cyclopentanesulfonamide_(6.1 g, 44.7%) as a yellow solid. LCMS: C₂₅H₁₉ClF₃N₅O₄S requires: 577.1, found: m/z=578.1 [M+H]⁺.

### Step-2: Synthesis of N-{3-[1-(4-amino-2,6-difluorophenyl)-3-(pyridin-4-yl)pyrazol-4-yl]-5-chloro-2-fluorophenyl}cyclopentanesulfonamide (4)

A mixture of Fe (2.94 g, 52.77 mmol, 5 equiv) and NH₄Cl (55 mg, 1.055 mmol, 0.1 equiv) in AcOH (2 mL) and H₂O (10 mL) was stirred at 80° C. for 5 min. To the above mixture was added N-{5-chloro-3-[1-(2,6-difluoro-4-nitrophenyl)-3-(pyridin-4-yl)pyrazol-4-yl]-2-fluorophenyl}cyclopentanesulfonamide (6.1 g, 10.55 mmol, 1 equiv) in EtOH (100 mL). The resulting mixture was stirred at 80° C. for 0.5 h. The resulting mixture was then concentrated under reduced pressure. The residue was purified by silica gel column chromatography eluted with PE:EtOAc (1:4) to afford N-{3-[1-(4-amino-2,6-difluorophenyl)-3-(pyridin-4-yl)pyrazol-4-yl]-5-chloro-2-fluorophenyl}cyclopentanesulfonamide (4.05 g, 70%) as a yellow solid. LCMS: C₂₅H₂₁ClF₃N₅O₂S requires: 547.1, found: m/z=548.2 [M+H]⁺.

### Step-3: Synthesis of N-{5-chloro-3-[1-(2,6-difluoro-4-iodophenyl)-3-(pyridin-4-yl)pyrazol-4-yl]-2-fluorophenyl}pyrrolidine-1-sulfonamide (5)

To a mixture of N-{3-[1-(4-amino-2,6-difluorophenyl)-3-(pyridin-4-yl)pyrazol-4-yl]-5-chloro-2-fluorophenyl}cyclopentanesulfonamide (4.0 g, 7.3 mmol, 1 equiv) and TsOH (3.82 g, 21.9 mmol, 3 equiv) in ACN (30 mL) was added NaNO₂ (1.53 g, 21.9 mmol, 3 equiv) in H₂O (3 mL) and KI (3.1 g, 18.25 mmol, 2.5 equiv) at 0° C. The resulting mixture was stirred at 0° C. for 10 min and then the reaction was warmed to room temperature. The mixture was stirred for additional 30 min at room temperature. The mixture was basified to pH=8 with saturated aqueous Na₂CO₃. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with PE:EtOAc (3:2) to afford N-{5-chloro-3-[1-(2,6-difluoro-4-iodophenyl)-3-(pyridin-4-yl)pyrazol-4-yl]-2-fluorophenyl}cyclopentanesulfonamide (982 mg, 20.4%) as a brown solid. LCMS: C₂₅H₁₉ClF₃1N₄O₂S requires: 657.9, found: m/z=658.9 [M+H]⁺.

### Step-4: Synthesis of tert-butyl 4-{4-[4-(5-chloro-3-cyclopentanesulfonamido-2-fluorophenyl)-3-(pyridin-4-yl)pyrazol-1-yl]-3,5-difluorophenyl}piperazine-1-carboxylate (6)

To a mixture of N-{5-chloro-3-[1-(2,6-difluoro-4-iodophenyl)-3-(pyridin-4-yl)pyrazol-4-yl]-2-fluorophenyl}cyclopentanesulfonamide (982 mg, 1.49 mmol, 1 equiv) and tert-butyl piperazine-1-carboxylate (694 mg, 3.725 mmol, 2.5 equiv) in dioxane (10 mL) was added Pd-PEPPSI-IHeptCl (145 mg, 0.149 mmol, 0.1 equiv) and Cs₂CO₃ (1.21 g, 3.725 mmol, 2.5 equiv). The resulting mixture was stirred at 90° C. for 4 h under a nitrogen atmosphere. The resulting mixture was then concentrated under reduced pressure. The residue was purified by silica gel column chromatography eluted with PE:EtOAc (1:2) to afford tert-butyl 4-{4-[4-(5-chloro-3-cyclopentanesulfonamido-2-fluorophenyl)-3-(pyridin-4-yl)pyrazol-1-yl]-3,5-difluorophenyl}piperazine-1-carboxylate (451 mg, 42.2%) as a brown solid. LCMS: C₃₄H₃₆ClF₃N₆O₄S requires: 716.2, found: m/z=717.3 [M+H]⁺.

### Step-5: Synthesis of N-(5-chloro-3-{1-[2,6-difluoro-4-(piperazin-1-yl)phenyl]-3-(pyridin-4-yl)pyrazol-4-yl}-2-fluorophenyl)cyclopentanesulfonamide TFA Salt (7)

A mixture of afford tert-butyl 4-{4-[4-(5-chloro-3-cyclopentanesulfonamido-2-fluorophenyl)-3-(pyridin-4-yl)pyrazol-1-yl]-3,5-difluorophenyl}piperazine-1-carboxylate (451 mg, 0.6288 mmol, 1 equiv) in TFA (5 mL) was stirred at 0° C. for one hour. The resulting mixture was then concentrated and lyophilized to afford N-(5-chloro-3-{1-[2,6-difluoro-4-(piperazin-1-yl)phenyl]-3-(pyridin-4-yl)pyrazol-4-yl}-2-fluorophenyl)cyclopentanesulfonamide trifluoroacetic acid salt (310 mg, crude) as a light yellow solid. LCMS: C₂₉H₂₈ClF₃N₆O₂S requires: 616.2; found: m/z=617.2 [M+H]⁺.

### Synthesis of ethylmethyl[(2-fluoro-3-{1-[3-fluoro-4-(piperazin-1-yl)phenyl]-3-(pyridin-4-yl)pyrazol-4-yl}phenyl)sulfamoyl]amine TFA Salt (6)

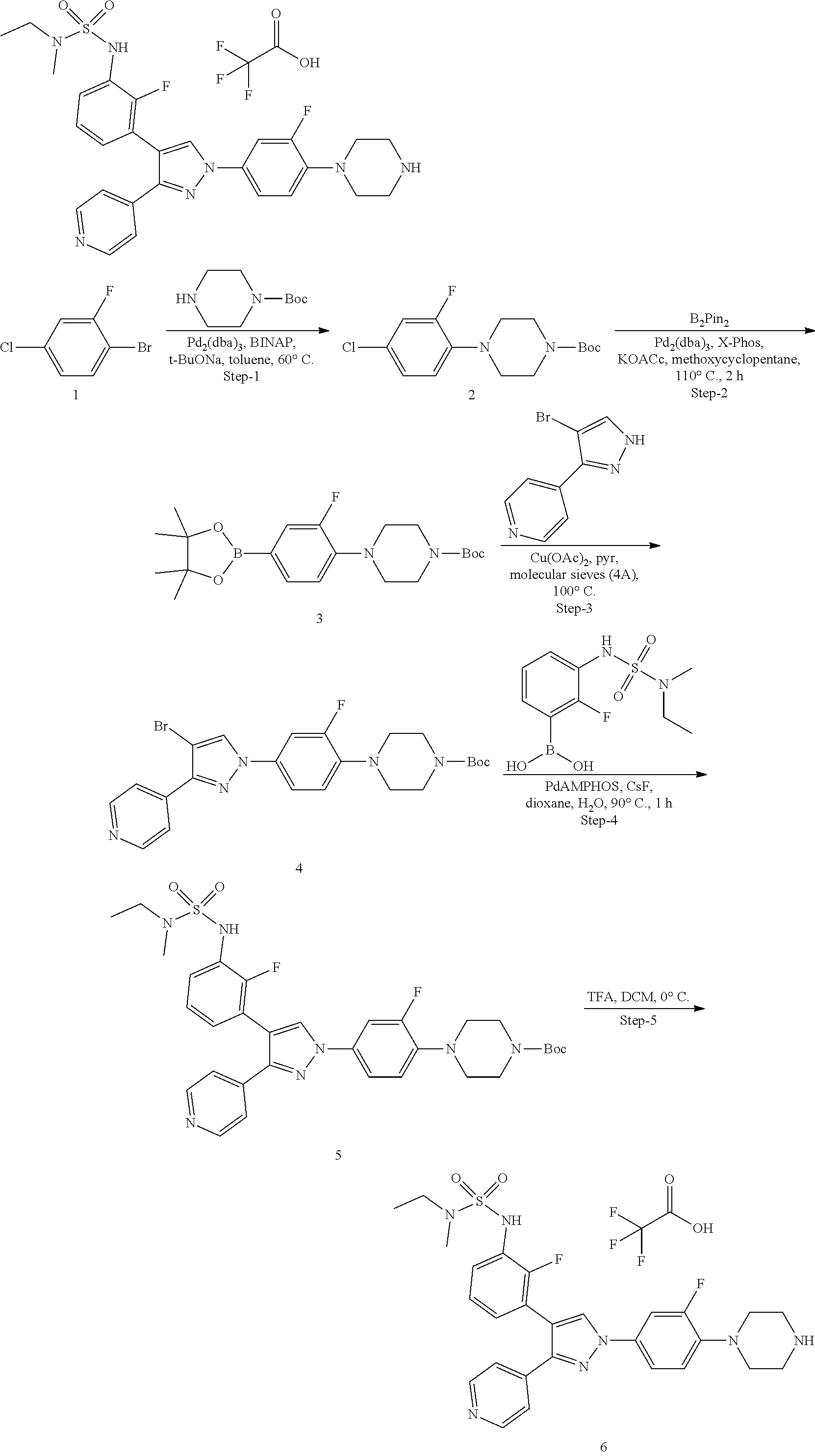

### Step-1: Synthesis of tert-butyl 4-(4-chloro-2-fluorophenyl)piperazine-1-carboxylate (2)

To a mixture of 1-bromo-4-chloro-2-fluorobenzene (3 g, 14.324 mmol, 1 equiv) and tert-butyl piperazine-1-carboxylate (2.67 g, 14.324 mmol, 1 equiv) in toluene (60 mL) was added Pd₂(dba)₃ (131 mg, 0.143 mmol, 0.01 equiv), BINAP (178 mg, 0.286 mmol, 0.02 equiv), and t-BuONa (1.93 g, 20.054 mmol, 1.4 equiv). The resulting mixture was stirred at 60° C. overnight under a nitrogen atmosphere. The resulting mixture was then concentrated under reduced pressure. The residue was purified by silica gel column chromatography eluted with PE:EtOAc (4:1) to afford tert-butyl 4-(4-chloro-2-fluorophenyl)piperazine-1-carboxylate (2 g, 39.92%) as an off-white solid. LCMS: C₁₅H₂₀ClFN₂O₂ requires: 314.1, found: m/z=315.1 [M+H]⁺.

### Step-2: Synthesis of tert-butyl 4-[2-fluoro-4-(4,4,5,5-tetramethyl-1,3,2-dioxaborolan-2-yl)phenyl]piperazine-1-carboxylate (3)

To a mixture of tert-butyl 4-(4-chloro-2-fluorophenyl)piperazine-1-carboxylate (2 g, 6.353 mmol, 1 equiv) and 4,4,5,5-tetramethyl-2-(4,4,5,5-tetramethyl-1,3,2-dioxaborolan-2-yl)-1,3,2-dioxaborolane (B₂Pin₂) (2.42 g, 9.529 mmol, 1.5 equiv) in methoxycyclopentane (30 mL) was added Pd₂(dba)₃ (175 mg, 0.191 mmol, 0.03 equiv), X-Phos (182 mg, 0.381 mmol, 0.06 equiv), and KOAc (1.87 g, 19.059 mmol, 3 equiv). The resulting mixture was stirred at 110° C. for 2 h under a nitrogen atmosphere. The resulting mixture was then concentrated under reduced pressure. The residue was purified by silica gel column chromatography eluted with PE:EtOAc (4:1) to afford tert-butyl 4-[2-fluoro-4-(4,4,5,5-tetramethyl-1,3,2-dioxaborolan-2-yl)phenyl]piperazine-1-carboxylate (2.5 g, 87.16%) as a brown oil. LCMS: C₂₁H₃₂BFN₂O₄ requires: 406.2, found: m/z=407.2 [M+H]⁺.

### Step-3: Synthesis of tert-butyl 4-{4-[4-bromo-3-(pyridin-4-yl)pyrazol-1-yl]-2-fluorophenyl}piperazine-1-carboxylate (4)

To a mixture of tert-butyl 4-[2-fluoro-4-(4,4,5,5-tetramethyl-1,3,2-dioxaborolan-2-yl)phenyl]piperazine-1-carboxylate (2.5 g, 6.153 mmol, 1 equiv) and 4-(4-bromo-1H-pyrazol-3-yl)pyridine (2.07 g, 9.229 mmol, 1.5 equiv) in pyridine (20 mL) was added Cu(OAc)₂ (2.24 g, 12.306 mmol, 2 equiv) and molecular sieves (4A) (2.07 g). The resulting mixture was stirred at 100° C. overnight under an oxygen atmosphere. The resulting mixture was then concentrated under reduced pressure. The residue was purified by silica gel column chromatography eluted with CH₂Cl₂:MeOH (20:1) to afford tert-butyl 4-{4-[4-bromo-3-(pyridin-4-yl)pyrazol-1-yl]-2-fluorophenyl}piperazine-1-carboxylate (2 g, 51.76%) as a brown semi-solid. LCMS: C₂₃H₂₅BrFN₅O₂ requires: 501.1, found: m/z=502.1 [M+H]⁺.

### Step-4: Synthesis of tert-butyl 4-{4-[4-(3-{[ethyl(methyl)sulfamoyl]amino}-2-fluorophenyl)-3-(pyridin-4-yl)pyrazol-1-yl]-2-fluorophenyl}piperazine-1-carboxylate

To a mixture of tert-butyl 4-{4-[4-bromo-3-(pyridin-4-yl)pyrazol-1-yl]-2-fluorophenyl}piperazine-1-carboxylate (1.5 g, 2.986 mmol, 1 equiv) and 3-{[ethyl(methyl)sulfamoyl]amino}-2-fluorophenylboronic acid (824 mg, 2.986 mmol, 1 equiv) in dioxane (10 mL) was added PdAMPHOS (423 mg, 0.597 mmol, 0.2 equiv) and CsF (907 mg, 5.972 mmol, 2 equiv) in H₂O (2 mL). The resulting mixture was stirred at 90° C. for one hour under a nitrogen atmosphere. The resulting mixture was then concentrated under reduced pressure. The residue was purified by C18 reverse phase column chromatography with ACN:H₂O (2:1) to afford tert-butyl 4-{4-[4-(3-{[ethyl(methyl)sulfamoyl]amino}-2-fluorophenyl)-3-(pyridin-4-yl)pyrazol-1-yl]-2-fluorophenyl}piperazine-1-carboxylate (500 mg, 24.33%) as an off-white solid. LCMS: C₃₂H₃₇F₂N₇O₄S requires: 653.3, found: m/z=654.3 [M+H]⁺.

### Step-5: Synthesis of ethylmethyl[(2-fluoro-3-{1-[3-fluoro-4-(piperazin-1-yl)phenyl]-3-(pyridin-4-yl)pyrazol-4-yl}phenyl)sulfamoyl]amine Trifluoroacetic Acid Salt

To a mixture of tert-butyl 4-{4-[4-(3-{[ethyl(methyl)sulfamoyl]amino}-2-fluorophenyl)-3-(pyridin-4-yl)pyrazol-1-yl]-2-fluorophenyl}piperazine-1-carboxylate (500 mg, 0.903 mmol, 1 equiv) in DCM (9 mL) was added TFA (3 mL) at 0° C. The resulting mixture was warmed and stirred at room temperature for one hour. The resulting mixture was then concentrated under reduced pressure. The product was then lyophilized under vacuum. This resulted in ethylmethyl[(2-fluoro-3-{1-[3-fluoro-4-(piperazin-1-yl)phenyl]-3-(pyridin-4-yl)pyrazol-4-yl}phenyl)sulfamoyl]amine trifluoroacetic acid salt (344.1 mg, crude) as a yellow solid. LCMS: C₂₇H₂₉F₂N₇O₂S requires: 553.2, found: m/z=554.2 [M+H]⁺. ¹HNMR (400 MHz, CD₃OD) δ 8.79-8.73 (m, 2H), 8.66-8.62 (m, 1H), 8.20-8.14 (m, 2H), 7.91-7.83 (m, 1H), 7.83-7.76 (m, 1H), 7.66-7.57 (m, 1H), 7.38-7.27 (m, 3H), 3.50-3.39 (m, 8H), 3.33-3.20 (m, 2H), 2.86-2.82 (m, 3H), 1.18-1.10 (m, 3H).

### Synthesis of ethylmethyl[(2-fluoro-3-{1-[2-fluoro-4-(piperazin-1-yl)phenyl]-3-(pyridin-4-yl)pyrazol-4-yl}phenyl)sulfamoyl]amine Trifluoroacetic Acid Salt (6)

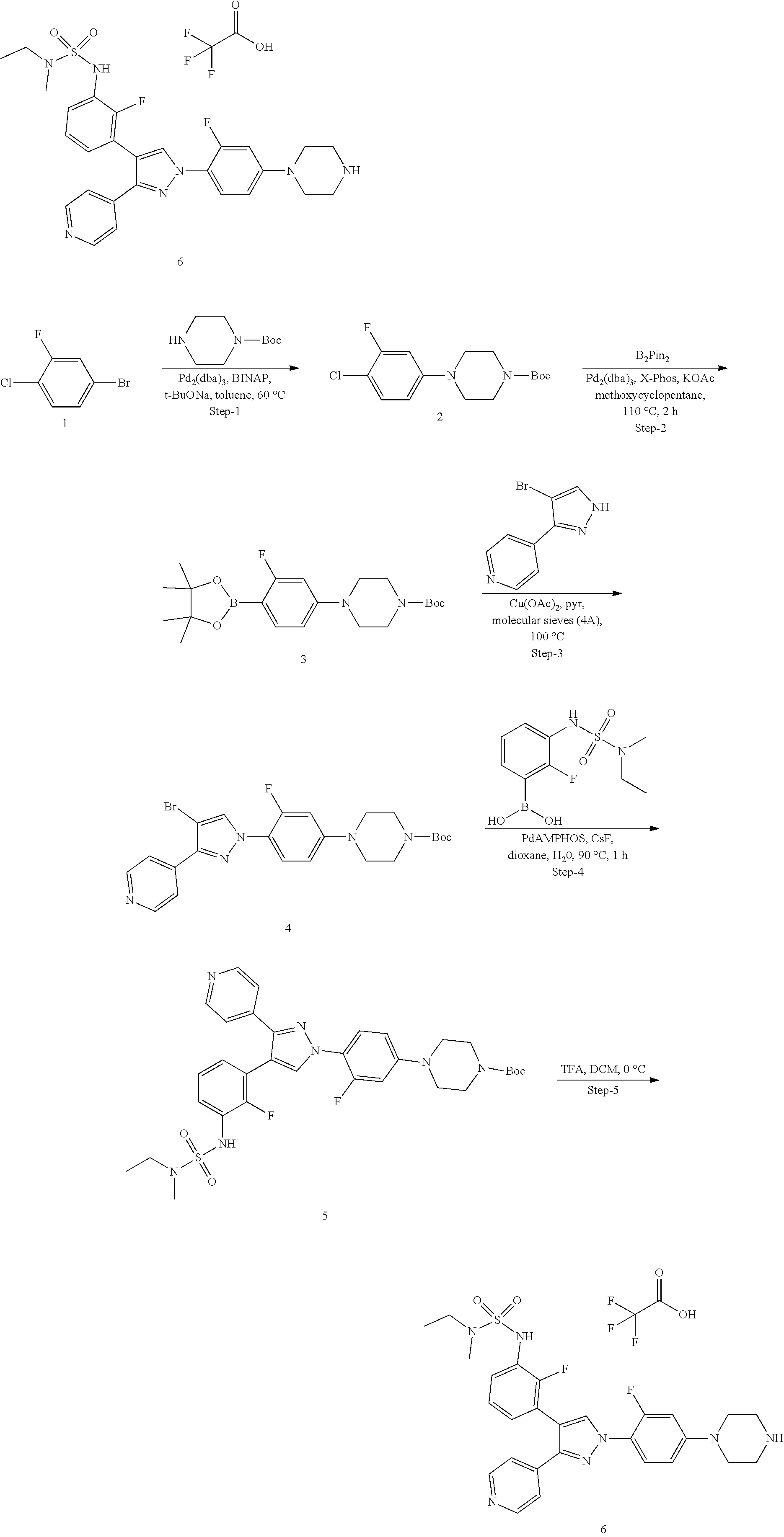

### Step-1: Synthesis of tert-butyl 4-(4-chloro-3-fluorophenyl)piperazine-1-carboxylate (2)

To a mixture of 4-bromo-1-chloro-2-fluorobenzene (3 g, 14.324 mmol, 1 equiv) and tert-butyl piperazine-1-carboxylate (2.67 g, 14.324 mmol, 1 equiv) in toluene (60 mL) was added Pd₂(dba)₃ (131 mg, 0.143 mmol, 0.01 equiv), BINAP (178 mg, 0.286 mmol, 0.02 equiv), and t-BuONa (1.93 g, 20.054 mmol, 1.4 equiv). The resulting mixture was stirred at 60° C. overnight under a nitrogen atmosphere. The resulting mixture was then concentrated under reduced pressure. The residue was purified by silica gel column chromatography eluted with PE:EtOAc (4:1) to afford tert-butyl 4-(4-chloro-3-fluorophenyl)piperazine-1-carboxylate (2 g, 39.92%) as an off-white solid. LCMS: C₁₅H₂₀ClFN₂O₂ requires: 314.1, found: m/z=315.1 [M+H]⁺.

### Step-2: Synthesis of tert-butyl 4-[3-fluoro-4-(4,4,5,5-tetramethyl-1,3,2-dioxaborolan-2-yl)phenyl]piperazine-1-carboxylate (3)

To a mixture of tert-butyl 4-(4-chloro-3-fluorophenyl)piperazine-1-carboxylate (2 g, 6.353 mmol, 1 equiv) and 4,4,5,5-tetramethyl-2-(4,4,5,5-tetramethyl-1,3,2-dioxaborolan-2-yl)-1,3,2-dioxaborolane (B₂Pin₂) (2.42 g, 9.529 mmol, 1.5 equiv) in methoxycyclopentane (30 mL) was added Pd₂(dba)₃ (175 mg, 0.191 mmol, 0.03 equiv), X-Phos (182 mg, 0.381 mmol, 0.06 equiv) and KOAc (1.87 g, 19.059 mmol, 3 equiv). The resulting mixture was stirred at 110° C. for 2 h under a nitrogen atmosphere. The resulting mixture was then concentrated under reduced pressure. The residue was purified by silica gel column chromatography eluted with PE:EtOAc (4:1) to afford tert-butyl 4-[3-fluoro-4-(4,4,5,5-tetramethyl-1,3,2-dioxaborolan-2-yl)phenyl]piperazine-1-carboxylate (1.5 g, 46.49%) as a brown oil. LCMS: C₂₁H₃₂BFN₂O₄ requires: 406.2, found: m/z=407.2 [M+H]⁺.

### Step-3: Synthesis of tert-butyl 4-(4-(4-bromo-3-(pyridin-4-yl)-1H-pyrazol-1-yl)-3-fluorophenyl)piperazine-1-carboxylate (4)

To a mixture of tert-butyl 4-[3-fluoro-4-(4,4,5,5-tetramethyl-1,3,2-dioxaborolan-2-yl)phenyl]piperazine-1-carboxylate (1.5 g, 3.692 mmol, 1 equiv) and 4-(4-bromo-1H-pyrazol-3-yl)pyridine (1.24 g, 5.538 mmol, 1.5 equiv) in pyridine (20 mL) was added Cu(OAc)₂ (1.34 g, 7.384 mmol, 2 equiv) and molecular sieves (4A) (1.5 g). The resulting mixture was stirred at 100° C. overnight under an oxygen atmosphere. The resulting mixture was then concentrated under reduced pressure. The residue was purified by silica gel column chromatography eluted with CH₂Cl₂:MeOH (20:1) to afford tert-butyl 4-(4-(4-bromo-3-(pyridin-4-yl)-1H-pyrazol-1-yl)-3-fluorophenyl)piperazine-1-carboxylate (500 mg, 22.91%) as a brown semi-solid. LCMS: C₂₃H₂₅BrFN₅O₂ requires: 501.1, found: m/z=502.1 [M+H]⁺.

### Step-4: Synthesis of tert-butyl 4-{4-[4-(3-{[ethyl(methyl)sulfamoyl]amino}-2-fluorophenyl)-3-(pyridin-4-yl)pyrazol-1-yl]-3-fluorophenyl}piperazine-1-carboxylate

To a mixture of tert-butyl 4-(4-(4-bromo-3-(pyridin-4-yl)-1H-pyrazol-1-yl)-3-fluorophenyl)piperazine-1-carboxylate (500 mg, 0.995 mmol, 1 equiv) and 3-{[ethyl(methyl)sulfamoyl]amino}-2-fluorophenylboronic acid (275 mg, 0.995 mmol, 1 equiv) in dioxane (10 mL) was added PdAMPHOS (141 mg, 0.199 mmol, 0.2 equiv) and CsF (302.36 mg, 1.990 mmol, 2 equiv) in H₂O (2 mL). The resulting mixture was stirred at 90° C. for 1 h under a nitrogen atmosphere. The resulting mixture was then concentrated under reduced pressure. The residue was purified by silica gel column chromatography eluted with PE:EtOAc (1:2) to afford tert-butyl 4-{4-[4-(3-{[ethyl(methyl)sulfamoyl]amino}-2-fluorophenyl)-3-(pyridin-4-yl)pyrazol-1-yl]-3-fluorophenyl}piperazine-1-carboxylate (450 mg, 55.33%) as a yellow solid. LCMS: C₃₂H₃₇F₂N₇O₄S requires: 653.3, found: m/z=654.3 [M+H]⁺.

### Step-5: Synthesis of ethylmethyl[(2-fluoro-3-{1-[2-fluoro-4-(piperazin-1-yl)phenyl]-3-(pyridin-4-yl)pyrazol-4-yl}phenyl)sulfamoyl]amine Trifluoroacetic Acid Salt

To a mixture of tert-butyl 4-{4-[4-(3-{[ethyl(methyl)sulfamoyl]amino}-2-fluorophenyl)-3-(pyridin-4-yl)pyrazol-1-yl]-3-fluorophenyl}piperazine-1-carboxylate (400 mg, 0.612 mmol, 1 equiv) in DCM (3 mL) was added TFA (1 mL) at 0° C. The resulting mixture was warmed and stirred at room temperature for one hour. The resulting mixture was then concentrated under reduced pressure. The residue was purified by C18 reverse phase column chromatography with ACN:H₂O (0.5% TFA) (1:3) to afford ethylmethyl[(2-fluoro-3-{1-[2-fluoro-4-(piperazin-1-yl)phenyl]-3-(pyridin-4-yl)pyrazol-4-yl}phenyl)sulfamoyl]amine trifluoroacetic acid salt (317.6 mg, 77.28%) as a yellow solid. LCMS: C₂₇H₂₉F₂N₇O₂S requires: 553.2, found: m/z=554.3 [M+H]⁺. ¹H NMR (400 MHz, DMSO-d₆) δ 9.67-9.62 (m, 1H), 8.85-8.81 (m, 2H), 8.66-8.60 (m, 2H), 8.46-8.41 (m, 1H), 7.80-7.71 (m, 1H), 7.57-7.51 (m, 2H), 7.54-7.45 (m, 1H), 7.31-7.20 (m, 2H), 7.20-7.12 (m, 1H), 7.05-6.96 (m, 1H), 3.55-3.48 (m, 4H), 3.32-3.22 (m, 4H), 3.13-3.03 (m, 2H), 2.70-2.66 (m, 3H), 1.05-0.97 (m, 3H).

### Synthesis of N-[2-fluoro-3-(1-{4-[(2R)-2-methylpiperazin-1-yl]phenyl}-3-(pyridin-4-yl)pyrazol-4-yl)phenyl]propane-1-sulfonamide Trifluoroacetic Acid Salt (8)

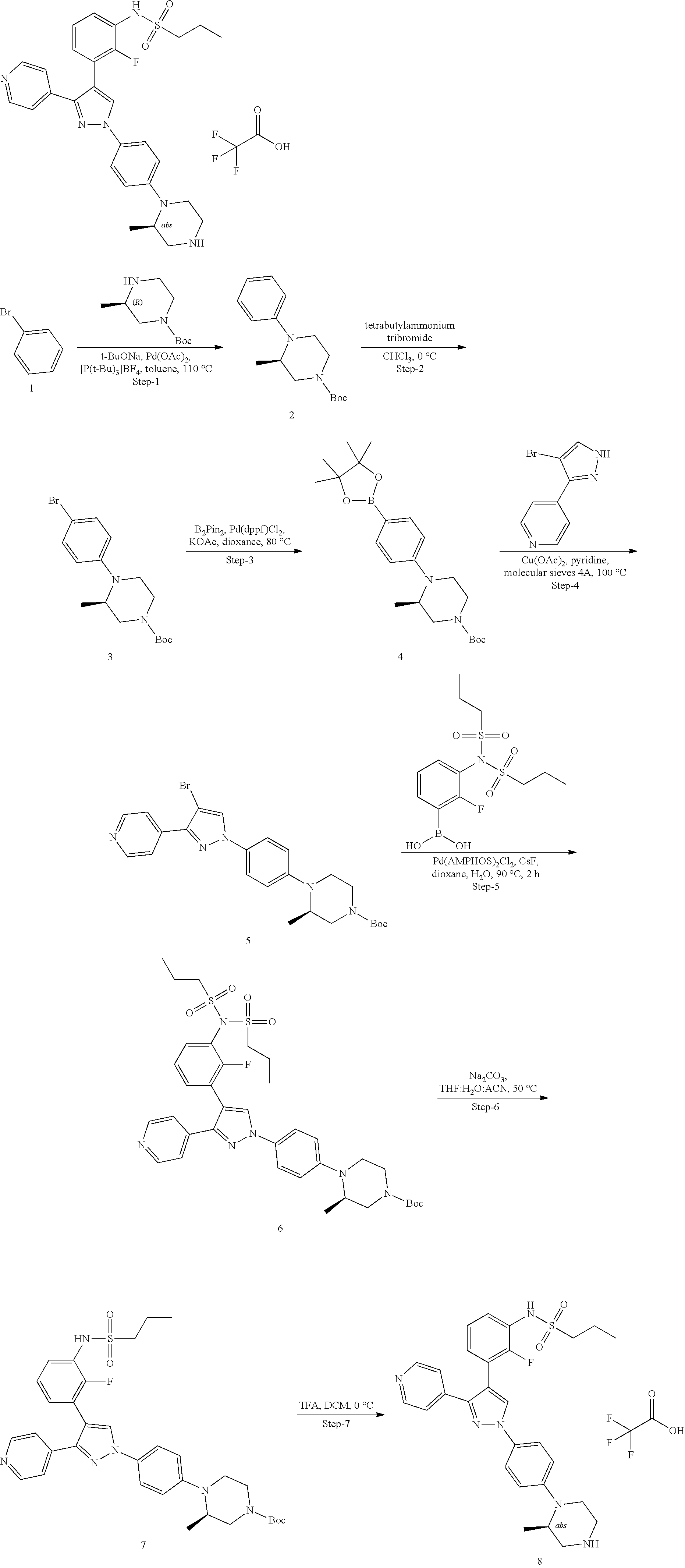

### Step-1: Synthesis of tert-butyl (3R)-3-methyl-4-phenylpiperazine-1-carboxylate (2)

To a mixture of bromobenzene (2 g, 12.762 mmol, 1.2 equiv) and tert-butyl (3R)-3-methylpiperazine-1-carboxylate (2.13 g, 10.635 mmol, 1 equiv) in toluene (20 mL) was added [P(t-Bu)₃]BF₄ (0.29 g, 0.851 mmol, 0.08 equiv), Pd(OAc)₂ (0.07 g, 0.319 mmol, 0.03 equiv), and t-BuONa (1.43 g, 14.889 mmol, 1.4 equiv). The resulting mixture was stirred at 110° C. overnight under a nitrogen atmosphere. The resulting mixture was then concentrated under vacuum. The residue was purified by silica gel column chromatography eluted with PE:EtOAc (3:1) to afford tert-butyl (3R)-3-methyl-4-phenylpiperazine-1-carboxylate (2.5 g, 76.55%) as a yellow oil. LCMS: C₁₆H₂₄N₂O₂ requires: 276.2, found: m/z=277.2 [M+H]⁺.

### Step-2: Synthesis of tert-butyl (3R)-4-(4-bromophenyl)-3-methylpiperazine-1-carboxylate (3)

To a mixture of tert-butyl (3R)-3-methyl-4-phenylpiperazine-1-carboxylate (2.4 g, 8.684 mmol, 1 equiv) in CHCl₃ (50 mL) was added tetrabutylammonium tribromide (4.19 g, 8.684 mmol, 1 equiv) at 0° C. The resulting mixture was warmed and stirred at room temperature for 0.5 h. The resulting mixture was then extracted with CHCl₃. The combined organic layers were washed with water, dried over anhydrous Na₂SO₄, filtered, and concentrated under vacuum. The residue was purified by silica gel column chromatography eluted with PE:EtOAc (1:1) to afford tert-butyl (3R)-4-(4-bromophenyl)-3-methylpiperazine-1-carboxylate (2.5 g, 72.93%) as a light yellow oil. LCMS: C₁₆H₂₃BrN₂O₂ requires: 354.1, found: m/z=355.2 [M+H]⁺.

### Step-3: Synthesis of tert-butyl (3R)-3-methyl-4-[4-(4,4,5,5-tetramethyl-1,3,2-dioxaborolan-2-yl)phenyl]piperazine-1-carboxylate (4)

To a mixture of tert-butyl (3R)-4-(4-bromophenyl)-3-methylpiperazine-1-carboxylate (2.47 g, 6.952 mmol, 1 equiv) and 4,4,5,5-tetramethyl-2-(4,4,5,5-tetramethyl-1,3,2-dioxaborolan-2-yl)-1,3,2-dioxaborolane (B₂Pin₂) (5.3 g, 20.856 mmol, 3 equiv) in 1,4-dioxane (50 mL) was added Pd(dppf)Cl₂ (0.57 g, 0.7 mmol, 0.1 equiv) and KOAc (1.36 g, 13.9 mmol, 2 equiv). The resulting mixture was stirred at 80° C. overnight under a nitrogen atmosphere. The resulting mixture was then concentrated under reduced pressure. The residue was purified by silica gel column chromatography eluted with PE:EtOAc (2:1) to afford tert-butyl (3R)-3-methyl-4-[4-(4,4,5,5-tetramethyl-1,3,2-dioxaborolan-2-yl)phenyl]piperazine-1-carboxylate (2.5 g, 80.44%) as a white solid. LCMS: C₂₂H₃₅BN₂O₄ requires: 402.3, found: m/z=403.2 [M+H]⁺.

### Step-4: Synthesis of tert-butyl (3R)-4-{4-[4-bromo-3-(pyridin-4-yl)pyrazol-1-yl]phenyl}-3-methylpiperazine-1-carboxylate (5)

To a mixture of tert-butyl (3R)-3-methyl-4-[4-(4,4,5,5-tetramethyl-1,3,2-dioxaborolan-2-yl)phenyl]piperazine-1-carboxylate (2.5 g, 6.214 mmol, 1 equiv) and 4-(4-bromo-1H-pyrazol-3-yl)pyridine (1.95 g, 8.7 mmol, 1.4 equiv) in pyridine (25 mL) was added Cu(OAc)₂ (2.26 g, 12.428 mmol, 2 equiv) and molecular sieves (4A) (2.5 g). The resulting mixture was stirred at 100° C. overnight under an oxygen atmosphere. The resulting mixture was then concentrated under reduced pressure. The residue was purified by silica gel column chromatography eluted with PE:EtOAc (3:1) to afford tert-butyl (3R)-4-{4-[4-bromo-3-(pyridin-4-yl)pyrazol-1-yl]phenyl}-3-methylpiperazine-1-carboxylate (2 g, 58.12%) as a light yellow solid. LCMS: C₂₄H₂₈BrN₅O₂ requires: 497.1, found: m/z=498.1 [M+H]⁺.

### Step-5: Synthesis of tert-butyl (3R)-4-[4-(4-{2-fluoro-3-[N-(propane-1-sulfonyl)-N-propane-1-sulfonamido]phenyl}-3-(pyridin-4-yl)pyrazol-1-yl)phenyl]-3-methylpiperazine-1-carboxylate (6)

To a mixture of tert-butyl (3R)-4-{4-[4-bromo-3-(pyridin-4-yl)pyrazol-1-yl]phenyl}-3-methylpiperazine-1-carboxylate (400 mg, 0.803 mmol, 1 equiv) and 2-fluoro-3-[N-(propane-1-sulfonyl)-N-propane-1-sulfonamido]phenylboronic acid (884 mg, 2.408 mmol, 3 equiv) in dioxane (10 mL) was added Pd(AMPHOS)₂Cl₂ (114 mg, 0.161 mmol, 0.2 equiv) and CsF (244 mg, 1.605 mmol, 2 equiv) in H₂O (2 mL). The resulting mixture was stirred at 90° C. for 2 h under a nitrogen atmosphere. The resulting mixture was then concentrated under reduced pressure. The residue was purified by silica gel column chromatography eluted with PE:EtOAc (1:2) to afford tert-butyl (3R)-4-[4-(4-{2-fluoro-3-[N-(propane-1-sulfonyl)-N-propane-1-sulfonamido]phenyl}-3-(pyridin-4-yl)pyrazol-1-yl)phenyl]-3-methylpiperazine-1-carboxylate (400 mg, 53.82%) as a dark yellow solid. LCMS: C₃₆H₄₅FN₆O₆S₂ requires: 740.3, found: m/z=741.2 [M+H]⁺.

### Step-6: Synthesis of tert-butyl (3R)-4-(4-{4-[2-fluoro-3-(propane-1-sulfonamido)phenyl]-3-(pyridin-4-yl)pyrazol-1-yl}phenyl)-3-methylpiperazine-1-carboxylate (7)

To a mixture of tert-butyl (3R)-4-[4-(4-{2-fluoro-3-[N-(propane-1-sulfonyl)-N-propane-1-sulfonamido]phenyl}-3-(pyridin-4-yl)pyrazol-1-yl)phenyl]-3-methylpiperazine-1-carboxylate (400 mg, 0.54 mmol, 1 equiv) in THF (10 mL) and ACN (10 mL) was added Na₂CO₃ (858 mg, 8.1 mmol, 15 equiv) in H₂O (10 mL). The resulting mixture was stirred at 50° C. overnight. The resulting mixture was then concentrated under reduced pressure. The residue was purified by silica gel column chromatography eluted with PE:EtOAc (5:1) to afford tert-butyl (3R)-4-(4-{4-[2-fluoro-3-(propane-1-sulfonamido)phenyl]-3-(pyridin-4-yl)pyrazol-1-yl}phenyl)-3-methylpiperazine-1-carboxylate (310 mg, 78.79%) as a yellow solid. LCMS: C₃₃H₃₉FN₆O₄S requires: 634.3, found: m/z=635.2 [M+H]⁺.

### Step-7: Synthesis of N-[2-fluoro-3-(1-{4-[(2R)-2-methylpiperazin-1-yl]phenyl}-3-(pyridin-4-yl)pyrazol-4-yl)phenyl]propane-1-sulfonamide Trifluoroacetic Acid Salt (8)

To a mixture of tert-butyl (3R)-4-(4-{4-[2-fluoro-3-(propane-1-sulfonamido)phenyl]-3-(pyridin-4-yl)pyrazol-1-yl}phenyl)-3-methylpiperazine-1-carboxylate (310 mg, 0.488 mmol, 1 equiv) in DCM (6 mL) was added TFA (2 mL) at 0° C. The resulting mixture was stirred at 0° C. for 2 h. The resulting mixture was then concentrated under reduced pressure and subjected to lyophilization. This resulted in N-[2-fluoro-3-(1-{4-[(2R)-2-methylpiperazin-1-yl]phenyl}-3-(pyridin-4-yl)pyrazol-4-yl)phenyl]propane-1-sulfonamide trifluoroacetic acid salt (309.0 mg, crude) as a yellow solid. LCMS: C₂₈H₃₁FN₆O₂S requires: 534.2, found: m/z=535.2 [M+H]⁺. ¹H NMR (400 MHz, Methanol-d₄) δ 8.78-8.72 (m, 2H), 8.60 (s, 1H), 8.24-8.17 (m, 2H), 7.98-7.89 (m, 2H), 7.65-7.56 (m, 1H), 7.45-7.23 (m, 4H), 4.16-4.08 (m, 1H), 3.58-3.44 (m, 3H), 3.41-3.34 (m, 2H), 3.31-3.26 (m, 1H), 3.19-3.11 (m, 2H), 1.94-1.80 (m, 2H), 1.21-1.15 (m, 3H), 1.10-1.01 (m, 3H).

### Synthesis of N-[2-fluoro-3-(1-{4-[(2S)-2-methylpiperazin-1-yl]phenyl}-3-(pyridin-4-yl)pyrazol-4-yl)phenyl]propane-1-sulfonamide Trifluoroacetic Acid Salt (8)

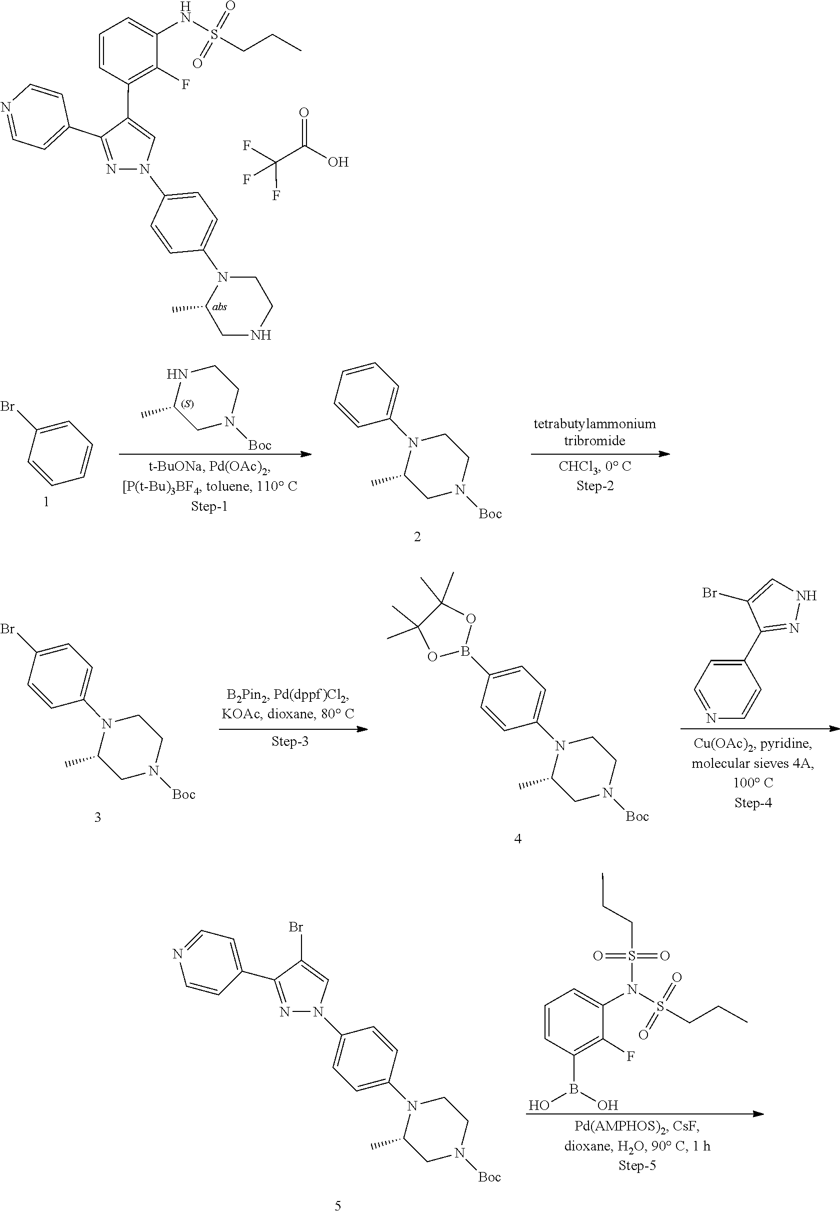

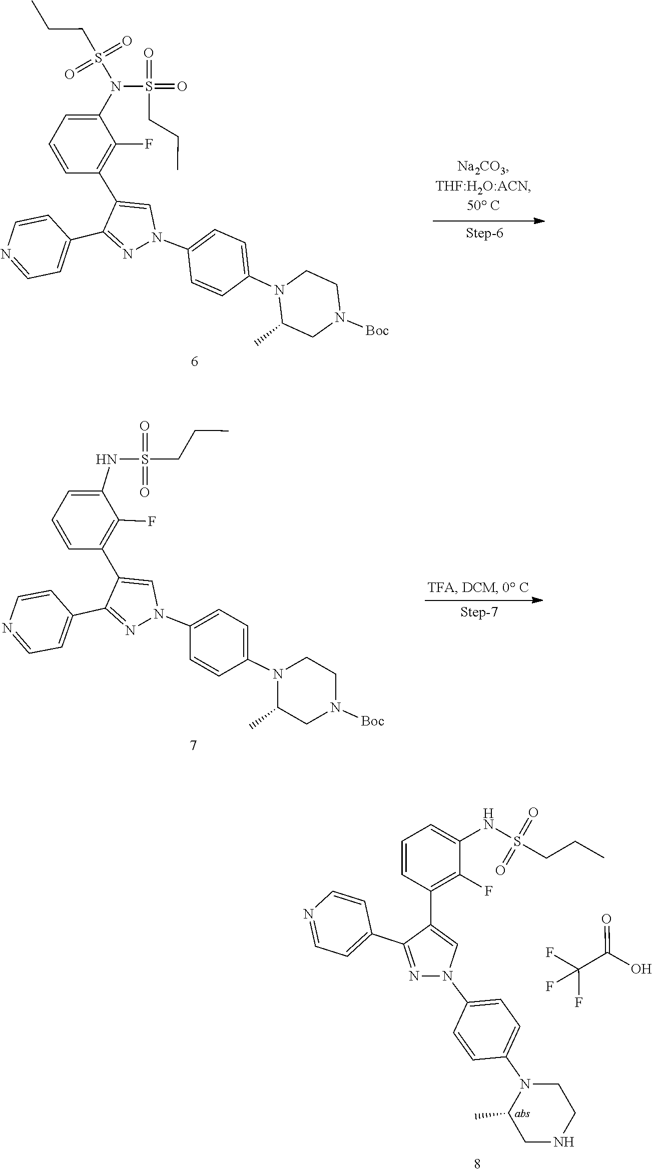 [SMILES: CCCS(=O)(=O)N(c1cccc(-c2cn(-c3ccc(N4CCN(C(=O)OC(C)(C)C)C[C@@H]4C)cc3)nc2-c2ccncc2)c1F)S(=O)(=O)CCC ; CCCS(=O)(=O)Nc1cccc(-c2cn(-c3ccc(N4CCN(C(=O)OC(C)(C)C)C[C@@H]4C)cc3)nc2-c2ccncc2)c1F ; CCCS(=O)(=O)Nc1cccc(-c2cn(-c3ccc(N4CCNC[C@@H]4C)cc3)nc2-c2ccncc2)c1F ; O=C(O)C(F)(F)F]

### Step-1: Synthesis of tert-butyl (3S)-3-methyl-4-phenylpiperazine-1-carboxylate (2)

To a mixture of bromobenzene (2.8 g, 17.975 mmol, 1.2 equiv) and tert-butyl (3S)-3-methylpiperazine-1-carboxylate (3 g, 14.979 mmol, 1 equiv) in toluene (30 mL) was added [P(t-Bu)₃]BF₄ (348 mg, 1.198 mmol, 0.08 equiv), Pd(OAc)₂ (101 mg, 0.449 mmol, 0.03 equiv), and t-BuOK (2 g, 20.971 mmol, 1.4 equiv). The resulting mixture was stirred at 110° C. overnight under a nitrogen atmosphere. The resulting mixture was then extracted with EtOAc. The combined organic layers were washed with water and dried over anhydrous Na₂SO₄. After filtration, the filtrate was concentrated under reduced pressure. The residue was purified by silica gel column chromatography eluted with PE:EtOAc (3:1) to afford tert-butyl (3S)-3-methyl-4-phenylpiperazine-1-carboxylate (3.5 g, 84.54%) as an off-white oil. LCMS: C₁₆H₂₄N₂O₂ requires: 276.2, found: m/z=277.2 [M+H]⁺.

### Step-2: Synthesis of tert-butyl (3S)-4-(4-bromophenyl)-3-methylpiperazine-1-carboxylate (3)

To a mixture of tert-butyl (3S)-3-methyl-4-phenylpiperazine-1-carboxylate (3.5 g, 12.664 mmol, 1 equiv) in CHCl₃ (40 mL) was added tetrabutylammonium tribromide (6.1 g, 12.664 mmol, 1 equiv) at 0° C. The resulting mixture was warmed and stirred at room temperature for 0.5 h. The resulting mixture was then extracted with CHCl₃. The combined organic layers were washed with water and dried over anhydrous Na₂SO₄. After filtration, the filtrate was concentrated under reduced pressure. The residue was purified by silica gel column chromatography eluted with PE:EtOAc (2:1) to afford tert-butyl (3S)-4-(4-bromophenyl)-3-methylpiperazine-1-carboxylate (4 g, 80.02%) as a white solid. LCMS: C₁₆H₂₃BrN₂02 requires: 354.2, found: m/z=355.2 [M+H]⁺.

### Step-3: Synthesis of tert-butyl (3S)-3-methyl-4-[4-(4,4,5,5-tetramethyl-1,3,2-dioxaborolan-2-yl)phenyl]piperazine-1-carboxylate (4)

To a mixture of tert-butyl (3S)-4-(4-bromophenyl)-3-methylpiperazine-1-carboxylate (4 g, 11.259 mmol, 1 equiv) and 4,4,5,5-tetramethyl-2-(4,4,5,5-tetramethyl-1,3,2-dioxaborolan-2-yl)-1,3,2-dioxaborolane (B₂Pin₂) (8.6 g, 33.777 mmol, 3 equiv) in 1,4-dioxane (40 mL) was added Pd(dppf)Cl₂ (917 mg, 1.126 mmol, 0.1 equiv) and KOAc (2.2 g, 22.518 mmol, 2 equiv). The resulting mixture was stirred at 80° C. overnight under a nitrogen atmosphere. The resulting mixture was then concentrated under reduced pressure. The residue was purified by silica gel column chromatography eluted with PE:EtOAc (2:1) to afford tert-butyl (3S)-3-methyl-4-[4-(4,4,5,5-tetramethyl-1,3,2-dioxaborolan-2-yl)phenyl]piperazine-1-carboxylate (4 g, 88.30%) as a white solid. LCMS: C₂₂H₃₅BN₂O₄ requires: 402.2, found: m/z=403.2 [M+H]⁺.

### Step-4: Synthesis of tert-butyl (3S)-4-{4-[4-bromo-3-(pyridin-4-yl)pyrazol-1-yl]phenyl}-3-methylpiperazine-1-carboxylate (5)

To a mixture of tert-butyl (3S)-3-methyl-4-[4-(4,4,5,5-tetramethyl-1,3,2-dioxaborolan-2-yl)phenyl]piperazine-1-carboxylate (4 g, 9.942 mmol, 1 equiv) and 4-(4-bromo-1H-pyrazol-3-yl)pyridine (3.1 g, 13.919 mmol, 1.4 equiv) in pyridine (40 mL) was added Cu(OAc)₂ (3.6 g, 19.884 mmol, 2 equiv) and molecular sieves (4A) (3.1 g). The resulting mixture was stirred at 100° C. overnight under an oxygen atmosphere. After filtration, the filtrate was concentrated under reduced pressure. The residue was purified by silica gel column chromatography eluted with PE:EtOAc (1:1) to afford tert-butyl (3S)-4-{4-[4-bromo-3-(pyridin-4-yl)pyrazol-1-yl]phenyl}-3-methylpiperazine-1-carboxylate (2 g, 36.33%) as a light yellow oil. LCMS: C₂₄H₂₈BrN₅O₂ requires: 497.1, found: m/z=498.1 [M+H]⁺.

### Step-5: Synthesis of tert-butyl (3S)-4-[4-(4-{2-fluoro-3-[N-(propane-1-sulfonyl)-N-propane-1-sulfonamido]phenyl}-3-(pyridin-4-yl)pyrazol-1-yl)phenyl]-3-methylpiperazine-1-carboxylate (6)

To a mixture of tert-butyl (3S)-4-{4-[4-bromo-3-(pyridin-4-yl)pyrazol-1-yl]phenyl}-3-methylpiperazine-1-carboxylate (500 mg, 1.003 mmol, 1 equiv) and 2-fluoro-3-[N-(propane-1-sulfonyl)-N-propane-1-sulfonamido]phenylboronic acid (1.5 g, 4.012 mmol, 4 equiv) in dioxane (10 mL) was added Pd(AMPHOS)₂Cl₂ (142 mg, 0.201 mmol, 0.2 equiv) and CsF (305 mg, 2.006 mmol, 2 equiv) in H₂O (2 mL). The resulting mixture was stirred at 90° C. for one hour under a nitrogen atmosphere. The resulting mixture was then concentrated under reduced pressure. This resulted in tert-butyl (3S)-4-[4-(4-{2-fluoro-3-[N-(propane-1-sulfonyl)-N-propane-1-sulfonamido]phenyl}-3-(pyridin-4-yl)pyrazol-1-yl)phenyl]-3-methylpiperazine-1-carboxylate (700 mg, crude) as a dark yellow solid. LCMS: C₃₆H₄₅FN₆O₆S₂ requires: 740.2, found: m/z=741.2 [M+H]⁺.

### Step-6: Synthesis of tert-butyl (3S)-4-(4-{4-[2-fluoro-3-(propane-1-sulfonamido)phenyl]-3-(pyridin-4-yl)pyrazol-1-yl}phenyl)-3-methylpiperazine-1-carboxylate (7)

To a mixture of tert-butyl (3S)-4-[4-(4-{2-fluoro-3-[N-(propane-1-sulfonyl)-N-propane-1-sulfonamido]phenyl}-3-(pyridin-4-yl)pyrazol-1-yl)phenyl]-3-methylpiperazine-1-carboxylate (700 mg, 0.945 mmol, 1 equiv) was added THF (20 mL), ACN (20 mL), and Na₂CO₃ (1.5 g, 14.175 mmol, 15 equiv) in H₂O (20 mL). The resulting mixture was stirred at 50° C. overnight. The resulting mixture was then concentrated under reduced pressure. The residue was purified by silica gel column chromatography eluted with PE:EtOAc (1:1) to afford tert-butyl (3S)-4-(4-{4-[2-fluoro-3-(propane-1-sulfonamido)phenyl]-3-(pyridin-4-yl)pyrazol-1-yl}phenyl)-3-methylpiperazine-1-carboxylate (250 mg, 37.52%) as a yellow solid. LCMS: C₃₃H₃₉FN₆O₄S requires: 634.2, found: m/z=635.2 [M+H]⁺.

### Step-7: Synthesis of N-[2-fluoro-3-(1-{4-[(2S)-2-methylpiperazin-1-yl]phenyl}-3-(pyridin-4-yl)pyrazol-4-yl)phenyl]propane-1-sulfonamide Trifluoroacetic Acid Salt (8)

To a mixture of tert-butyl (3S)-4-(4-{4-[2-fluoro-3-(propane-1-sulfonamido)phenyl]-3-(pyridin-4-yl)pyrazol-1-yl}phenyl)-3-methylpiperazine-1-carboxylate (240 mg, 0.378 mmol, 1 equiv) in DCM (6 mL) was added TFA (2 mL) at 0° C. The resulting mixture was warmed and stirred at room temperature for one hour. The resulting mixture was concentrated under reduced pressure. This resulted in N-[2-fluoro-3-(1-{4-[(2S)-2-methylpiperazin-1-yl]phenyl}-3-(pyridin-4-yl)pyrazol-4-yl)phenyl]propane-1-sulfonamide trifluoroacetic acid salt (215.5 mg, 87.51%) as a yellow solid. LCMS: C₂₈H₃₁FN₆O₂S requires: 534.2, found: m/z=535.2 [M+H]⁺. ¹H NMR (400 MHz, Methanol-d₄) δ 8.78-8.72 (m, 2H), 8.60 (s, 1H), 8.24-8.17 (m, 2H), 7.98-7.89 (m, 2H), 7.65-7.56 (m, 1H), 7.45-7.32 (m, 2H), 7.31-7.23 (m, 2H), 4.16-4.08 (m, 1H), 3.58-3.44 (m, 3H), 3.41-3.34 (m, 2H), 3.31-3.26 (m, 1H), 3.19-3.11 (m, 2H), 1.94-1.80 (m, 2H), 1.21-1.15 (m, 3H), 1.10-1.01 (m, 3H).

### Synthesis of N-[2-fluoro-3-(1-{4-[(3R)-3-methylpiperazin-1-yl]phenyl}-3-(pyridin-4-yl)pyrazol-4-yl)phenyl]propane-1-sulfonamide Trifluoroacetic Acid Salt (7)

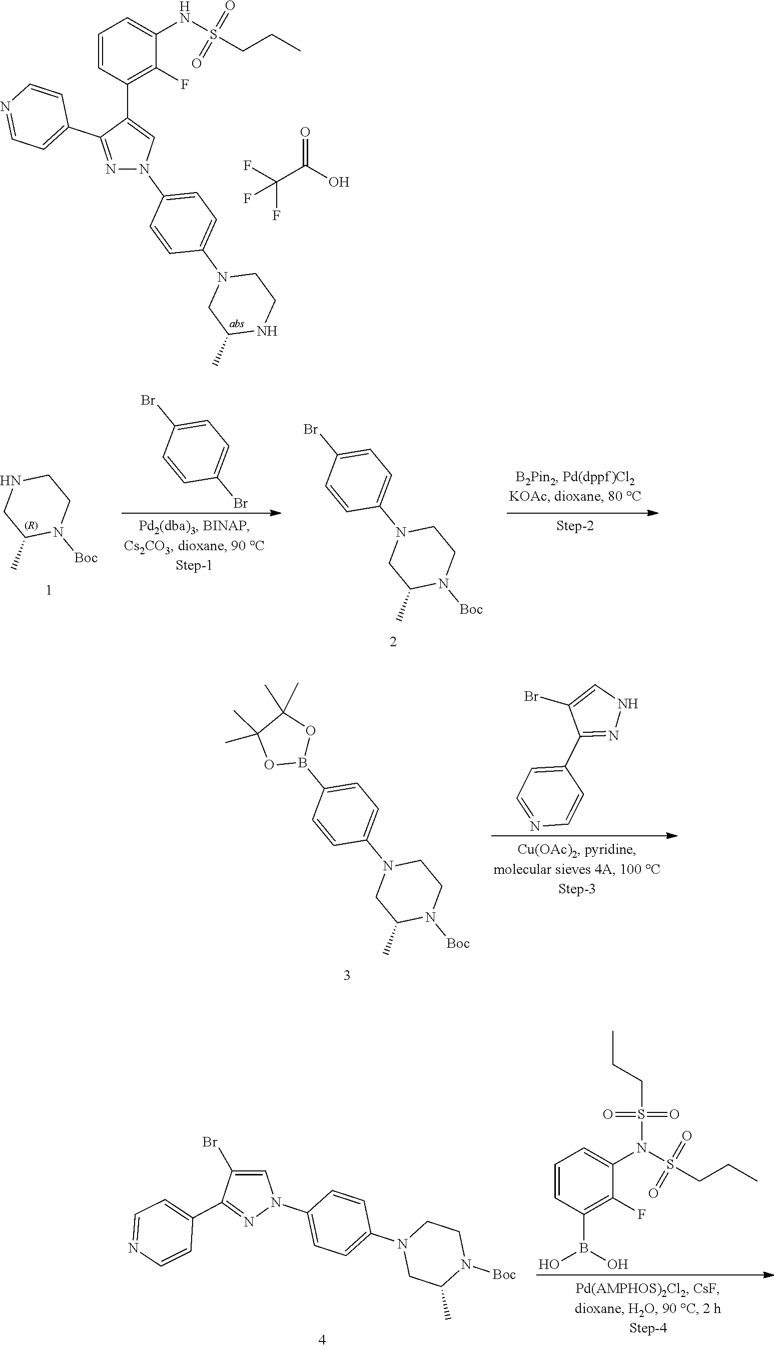

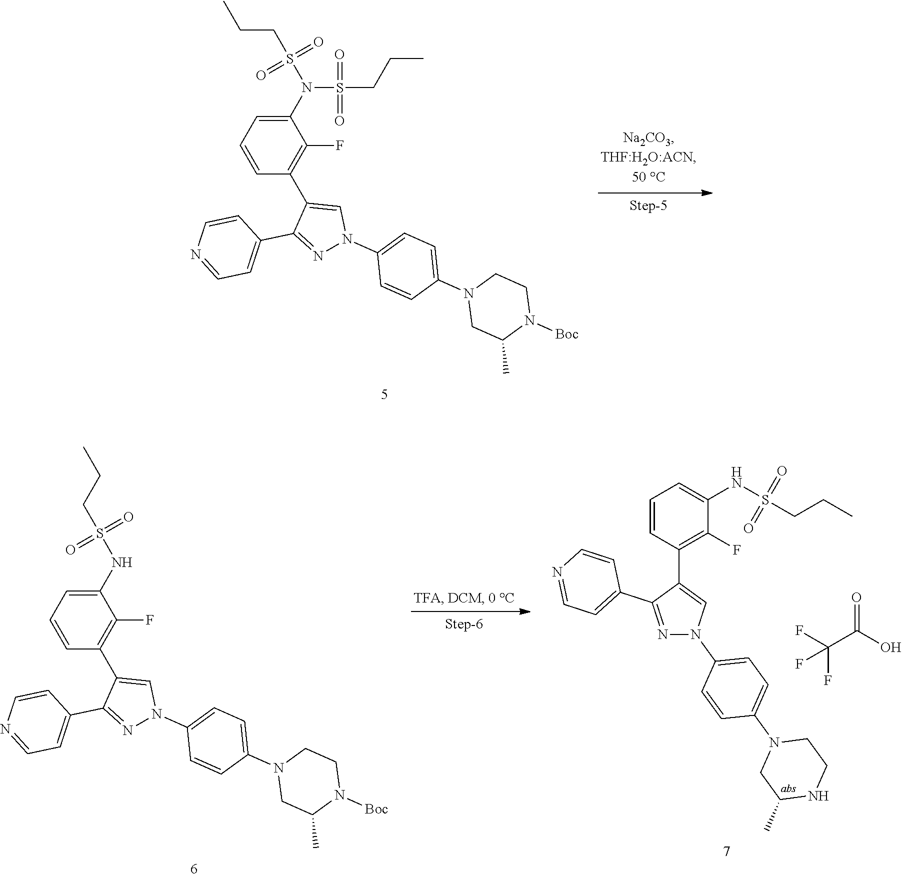

### Step-1: Synthesis of tert-butyl (2R)-4-(4-bromophenyl)-2-methylpiperazine-1-carboxylate (2)

To a mixture of tert-butyl (2R)-2-methylpiperazine-1-carboxylate (5 g, 24.965 mmol, 1 equiv) and 1,4-dibromobenzene (14.72 g, 62.413 mmol, 2.5 equiv) in 1,4-dioxane (50 mL) was added Pd₂(dba)₃ (1.14 g, 1.248 mmol, 0.05 equiv), BINAP (0.78 g, 1.248 mmol, 0.05 equiv), and Cs₂CO₃ (12.2 g, 37.447 mmol, 1.5 equiv). The resulting mixture was stirred at 90° C. overnight under a nitrogen atmosphere. The resulting mixture was then concentrated under reduced pressure. The residue was purified by reverse phase flash chromatography eluted with ACN:H₂O (1:1) to afford tert-butyl (2R)-4-(4-bromophenyl)-2-methylpiperazine-1-carboxylate (4.97 g, 56%) as a white solid. LCMS: C₁₆H₂₃BrN₂O₂ requires: 354.1, found: m/z=355.2 [M+H]⁺.

### Step-2: Synthesis of tert-butyl (2R)-2-methyl-4-[4-(4,4,5,5-tetramethyl-1,3,2-dioxaborolan-2-yl)phenyl]piperazine-1-carboxylate (3)

To a mixture of tert-butyl (2R)-4-(4-bromophenyl)-2-methylpiperazine-1-carboxylate (2 g, 5.629 mmol, 1 equiv) and B₂Pin₂ (4.29 g, 16.887 mmol, 3 equiv) in 1,4-dioxane (20 mL) was added Pd(dppf)Cl₂ (412 mg, 0.563 mmol, 0.1 equiv) and KOAc (1.1 g, 11.258 mmol, 2 equiv). The resulting mixture was stirred at 80° C. overnight under a nitrogen atmosphere. The resulting mixture was concentrated under reduced pressure. The residue was purified by silica gel column chromatography eluted with PE:EtOAc (1:1) to afford tert-butyl (2R)-2-methyl-4-[4-(4,4,5,5-tetramethyl-1,3,2-dioxaborolan-2-yl)phenyl]piperazine-1-carboxylate (2 g, 79.47%) as a white solid. LCMS: C₂₂H₃₅BN₂O₄ requires: 402.3, found: m/z=403.2 [M+H]⁺.

### Step-3: Synthesis of tert-butyl (2R)-4-{4-[4-bromo-3-(pyridin-4-yl)pyrazol-1-yl]phenyl}-2-methylpiperazine-1-carboxylate (4)

To a mixture of tert-butyl (2R)-2-methyl-4-[4-(4,4,5,5-tetramethyl-1,3,2-dioxaborolan-2-yl)phenyl]piperazine-1-carboxylate (2 g, 4.971 mmol, 1 equiv) and 4-(4-bromo-1H-pyrazol-3-yl)pyridine (1.56 g, 6.959 mmol, 1.4 equiv) in pyridine (20 mL) was added Cu(OAc)₂ (1.81 g, 9.942 mmol, 2 equiv) and molecular sieves (4A) (2 g). The resulting mixture was stirred at 100° C. overnight under an oxygen atmosphere. The resulting mixture was extracted with EtOAc. The combined organic layers were washed with water and dried over anhydrous Na₂SO₄. After filtration, the filtrate was concentrated under reduced pressure. The residue was purified by reverse phase flash chromatography eluted with ACN:H₂O (1:1) to afford tert-butyl (2R)-4-{4-[4-bromo-3-(pyridin-4-yl)pyrazol-1-yl]phenyl}-2-methylpiperazine-1-carboxylate (900 mg, 26.52%) as a yellow solid. LCMS: C₂₄H₂₈BrN₅O₂ requires: 497.1, found: m/z=498.1 [M+H]⁺.

### Step-4: Synthesis of tert-butyl (2R)-4-[4-(4-{2-fluoro-3-[N-(propane-1-sulfonyl)-N-propane-1-sulfonamido]phenyl}-3-(pyridin-4-yl)pyrazol-1-yl)phenyl]-2-methylpiperazine-1-carboxylate (5)

To a mixture of tert-butyl (2R)-4-{4-[4-bromo-3-(pyridin-4-yl)pyrazol-1-yl]phenyl}-2-methylpiperazine-1-carboxylate (400 mg, 0.803 mmol, 1 equiv) and 2-fluoro-3-[N-(propane-1-sulfonyl)-N-propane-1-sulfonamido]phenylboronic acid (1179 mg, 3.21 mmol, 4 equiv) in dioxane (10 mL) was added Pd(AMPHOS)₂Cl₂ (114 mg, 0.161 mmol, 0.2 equiv) and CsF (2441 mg, 1.606 mmol, 2 equiv) in H₂O (2 mL). The resulting mixture was stirred at 90° C. for 2 h under a nitrogen atmosphere. The resulting mixture was then concentrated under reduced pressure. The residue was purified by silica gel column chromatography eluted with PE:EtOAc (1:3) to afford tert-butyl (2R)-4-[4-(4-{2-fluoro-3-[N-(propane-1-sulfonyl)-N-propane-1-sulfonamido]phenyl}-3-(pyridin-4-yl)pyrazol-1-yl)phenyl]-2-methylpiperazine-1-carboxylate (400 mg, 61.89%) as a yellow solid. LCMS: C₃₆H₄₅FN₆O₆S₂ requires: 740.3, found: m/z=741.2 [M+H]⁺.

### Step-5: Synthesis of tert-butyl (2R)-4-(4-{4-[2-fluoro-3-(propane-1-sulfonamido)phenyl]-3-(pyridin-4-yl)pyrazol-1-yl}phenyl)-2-methylpiperazine-1-carboxylate (6)

To a mixture of tert-butyl (2R)-4-[4-(4-{2-fluoro-3-[N-(propane-1-sulfonyl)-N-propane-1-sulfonamido]phenyl}-3-(pyridin-4-yl)pyrazol-1-yl)phenyl]-2-methylpiperazine-1-carboxylate (400 mg, 0.54 mmol, 1 equiv) was added THF (10 mL), ACN (10 mL), and Na₂CO₃ (858 mg, 8.1 mmol, 15 equiv) in H₂O (10 mL). The resulting mixture was stirred at 50° C. overnight. The resulting mixture was then extracted with EtOAc. The combined organic layers were washed with water and dried over anhydrous Na₂SO₄. After filtration, the filtrate was concentrated under reduced pressure. The residue was purified by reverse phase flash chromatography eluted with ACN:H₂O (1:1) to afford tert-butyl (2R)-4-(4-{4-[2-fluoro-3-(propane-1-sulfonamido)phenyl]-3-(pyridin-4-yl)pyrazol-1-yl}phenyl)-2-methylpiperazine-1-carboxylate (220 mg, 57.78%) as a yellow solid. LCMS: C₃₃H₃₉FN₆O₄S requires: 634.3, found: m/z=635.2 [M+H]⁺.

### Step-6: Synthesis of N-[2-fluoro-3-(1-{4-[(3R)-3-methylpiperazin-1-yl]phenyl}-3-(pyridin-4-yl)pyrazol-4-yl)phenyl]propane-1-sulfonamide Trifluoroacetic Acid Salt (7)

To a mixture of tert-butyl (2R)-4-(4-{4-[2-fluoro-3-(propane-1-sulfonamido)phenyl]-3-(pyridin-4-yl)pyrazol-1-yl}phenyl)-2-methylpiperazine-1-carboxylate (215 mg, 0.339 mmol, 1 equiv) in DCM (6 mL) was added TFA (2 mL) at 0° C. The resulting mixture was stirred at 0° C. for 2 h. The resulting mixture was then concentrated under reduced pressure and subjected to lyophilization. This resulted in N-[2-fluoro-3-(1-{4-[(3R)-3-methylpiperazin-1-yl]phenyl}-3-(pyridin-4-yl)pyrazol-4-yl)phenyl]propane-1-sulfonamide trifluoroacetic acid salt (217.9 mg, crude) as a yellow solid. LCMS: C₂₈H₃₁FN₆O₂S requires: 534.2, found: m/z=535.2 [M+H]⁺. ¹H NMR (400 MHz, Methanol-d₄) δ 8.77-8.68 (m, 2H), 8.57 (s, 1H), 8.20-8.11 (m, 2H), 7.94-7.86 (m, 2H), 7.64-7.56 (m, 1H), 7.44-7.30 (m, 2H), 7.27-7.22 (m, 2H), 4.01-3.83 (m, 2H), 3.66-3.35 (m, 2H), 3.34 (s, 1H), 3.20-3.07 (m, 3H), 2.97-2.84 (m, 1H), 1.92-1.80 (m, 2H), 1.50-1.39 (m, 3H), 1.12-1.00 (m, 3H).

### Synthesis of ethylmethyl[(2-fluoro-3-{1-[6-(piperazin-1-yl)pyridin-3-yl]-3-(pyridin-4-yl)pyrazol-4-yl}phenyl)sulfamoyl]amine Trifluoroacetic Acid Salt (4)

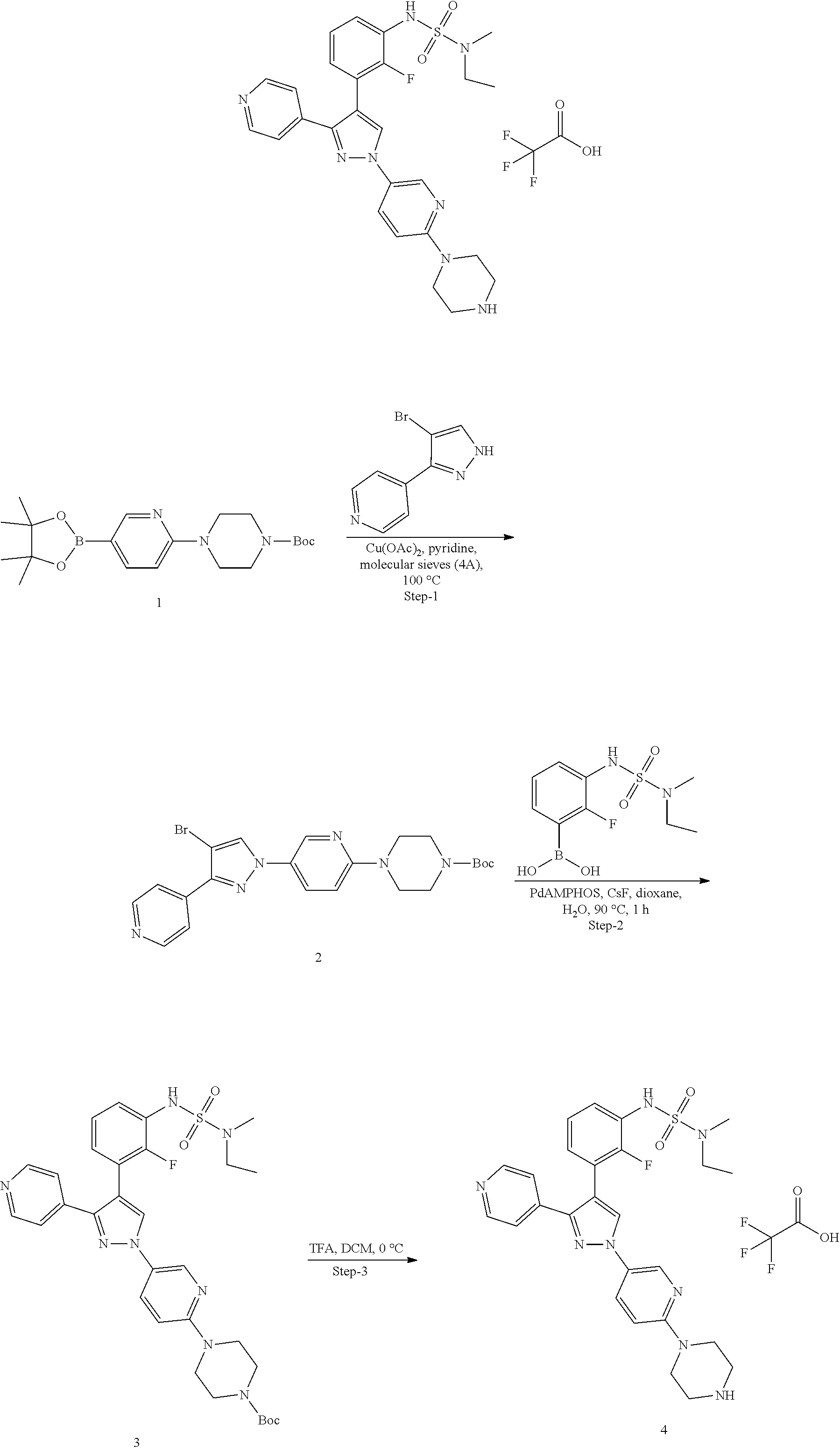

### Step-1: Synthesis of tert-butyl 4-{5-[4-bromo-3-(pyridin-4-yl)pyrazol-1-yl]pyridin-2-yl}piperazine-1-carboxylate (2)

To a mixture of tert-butyl 4-[5-(4,4,5,5-tetramethyl-1,3,2-dioxaborolan-2-yl)pyridin-2-yl]piperazine-1-carboxylate (2 g, 5.137 mmol, 1 equiv) and 4-(4-bromo-1H-pyrazol-3-yl)pyridine (1.7 g, 7.705 mmol, 1.5 equiv) in pyridine (20 mL) was added Cu(OAc)₂ (1.87 g, 10.274 mmol, 2 equiv) and molecular sieves (4A) (1.7 g). The resulting mixture was stirred at 100° C. overnight under an oxygen atmosphere. The resulting mixture was then concentrated under reduced pressure. The residue was purified by silica gel column chromatography eluted with PE:EtOAc (1:1) to afford tert-butyl 4-{5-[4-bromo-3-(pyridin-4-yl)pyrazol-1-yl]pyridin-2-yl}piperazine-1-carboxylate (1.5 g, 60.15%) as a yellow solid. LCMS: C₂₂H₂₅BrN₆O₂ requires: 484.1, found: m/z=485.1 [M+H]⁺.

### Step-2: Synthesis of tert-butyl 4-{5-[4-(3-{[ethyl(methyl)sulfamoyl]amino}-2-fluorophenyl)-3-(pyridin-4-yl)pyrazol-1-yl]pyridin-2-yl}piperazine-1-carboxylate (3)

To a mixture of tert-butyl 4-{5-[4-bromo-3-(pyridin-4-yl)pyrazol-1-yl]pyridin-2-yl}piperazine-1-carboxylate (500 mg, 1.03 mmol, 1 equiv) and 3-{[ethyl(methyl)sulfamoyl]amino}-2-fluorophenylboronic acid (341 mg, 1.236 mmol, 1.2 equiv) in dioxane (5 mL) was added PdAMPHOS (73 mg, 0.103 mmol, 0.1 equiv) and CsF (313 mg, 2.06 mmol, 2 equiv) in H₂O (1 mL). The resulting mixture was stirred at 90° C. for one hour under a nitrogen atmosphere. The resulting mixture was then concentrated under reduced pressure. The residue was purified by reverse phase flash chromatography eluted with ACN:H₂O (1:2) to afford tert-butyl 4-{5-[4-(3-{[ethyl(methyl)sulfamoyl]amino}-2-fluorophenyl)-3-(pyridin-4-yl)pyrazol-1-yl]pyridin-2-yl}piperazine-1-carboxylate (300 mg, 41.16%) as a white solid. LCMS: C₃₁H₃₇FN₈O₄S requires: 636.3, found: m/z=637.3 [M+H]⁺.

### Step-3: Synthesis of ethylmethyl[(2-fluoro-3-{1-[6-(piperazin-1-yl)pyridin-3-yl]-3-(pyridin-4-yl)pyrazol-4-yl}phenyl)sulfamoyl]amine trifluoroacetic acid salt (4)

To a mixture of tert-butyl 4-{5-[4-(3-{[ethyl(methyl)sulfamoyl]amino}-2-fluorophenyl)-3-(pyridin-4-yl)pyrazol-1-yl]pyridin-2-yl}piperazine-1-carboxylate (300 mg, 0.471 mmol, 1 equiv) in DCM (6 mL) was added TFA (2 mL) at 0° C. The resulting mixture was stirred at room temperature for one hour. The resulting mixture was then concentrated under reduced pressure and subjected to lyophilization. This resulted in ethylmethyl[(2-fluoro-3-{1-[6-(piperazin-1-yl)pyridin-3-yl]-3-(pyridin-4-yl)pyrazol-4-yl}phenyl)sulfamoyl]amine trifluoroacetic acid salt (233.3 mg, crude) as a yellow solid. LCMS: C₂₆H₂₉FN₈O₂S requires: 536, found: m/z=537 [M+H]⁺. ¹H NMR (400 MHz, Methanol-d₄) δ 8.86-8.72 (m, 3H), 8.59 (s, 1H), 8.26-8.13 (m, 3H), 7.64-7.56 (m, 1H), 7.38-7.29 (m, 2H), 7.19-7.10 (m, 1H), 3.99-3.88 (m, 4H), 3.44-3.37 (m, 4H), 3.29-3.21 (m, 2H), 2.84 (s, 3H), 1.23-1.10 (m, 3H).

### Synthesis of N-(2,5-difluoro-3-{1-[4-(piperazin-1-yl)phenyl]-3-(pyridin-4-yl)pyrazol-4-yl}phenyl)propane-1-sulfonamide Trifluoroacetic Acid Salt (6)

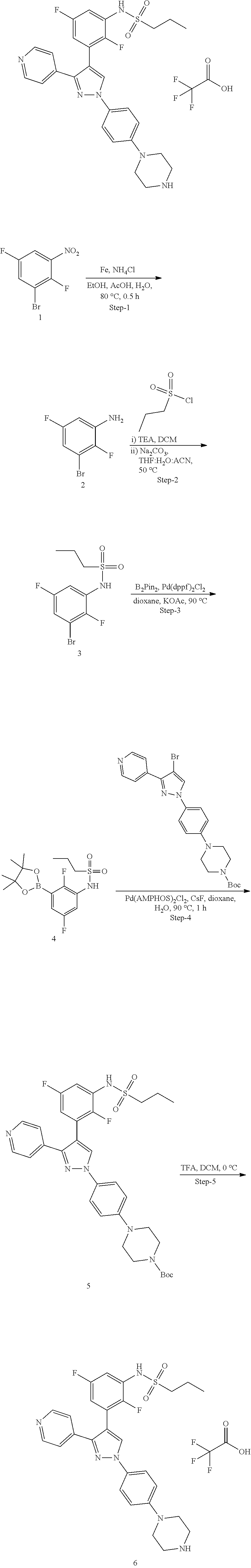

### Step-1: Synthesis of 3-bromo-2,5-difluoroaniline (2)

To a mixture of Fe (5.8 g, 105.045 mmol, 5 equiv) in AcOH (2 mL) and H₂O (10 mL) was added NH₄Cl (112 mg, 2.101 mmol, 0.1 equiv). The resulting mixture was stirred at 80° C. for 5 min. To the above mixture was added 1-bromo-2,5-difluoro-3-nitrobenzene (5 g, 21.009 mmol, 1 equiv) in EtOH (100 mL). The resulting mixture was stirred at 80° C. for 0.5 h. The resulting mixture was then concentrated under reduced pressure. The residue was purified by silica gel column chromatography eluted with PE:EtOAc (2:1) to afford 3-bromo-2,5-difluoroaniline (4 g, 82.38%) as an off-white oil. LCMS: C₆H₄BrF₂N requires: 207.2, found: m/z=208.3 [M+H]⁺.

### Step-2: Synthesis of N-(3-bromo-2,5-difluorophenyl)propane-1-sulfonamide (3)

To a mixture of 3-bromo-2,5-difluoroaniline (500 mg, 2.404 mmol, 1 equiv) and TEA (729 mg, 7.212 mmol, 3 equiv) in DCM (20 mL) was added propane-1-sulfonyl chloride (1.1 g, 7.212 mmol, 3 equiv) at 0° C. The resulting mixture was stirred at room temperature for one hour. The resulting mixture was then concentrated under reduced pressure. To the above mixture was then added THF (50 mL), ACN (50 mL), and Na₂CO₃ (3.8 g, 36.060 mmol, 15 equiv) in H₂O (50 mL). The resulting mixture was stirred at 50° C. overnight. The resulting mixture was then concentrated under reduced pressure. The residue was purified by silica gel column chromatography eluted with PE:EtOAc (1:1) to afford N-(3-bromo-2,5-difluorophenyl)propane-1-sulfonamide (700 mg, 83.43%) as a white solid. LCMS: C₉H₁₀BrF₂NO₂S requires: 313.2, found: m/z=314.3 [M+H]⁺.

### Step-3: Synthesis of N-[2,5-difluoro-3-(4,4,5,5-tetramethyl-1,3,2-dioxaborolan-2-yl)phenyl]propane-1-sulfonamide (4)

To a mixture of N-(3-bromo-2,5-difluorophenyl)propane-1-sulfonamide (700 mg, 2.228 mmol, 1 equiv) and 4,4,5,5-tetramethyl-2-(4,4,5,5-tetramethyl-1,3,2-dioxaborolan-2-yl)-1,3,2-dioxaborolane (B₂Pin₂) (1.1 g, 4.456 mmol, 2 equiv) in 1,4-dioxane (10 mL) was added Pd(dppf)Cl₂ (181 mg, 0.223 mmol, 0.1 equiv) and KOAc (437 mg, 4.456 mmol, 2 equiv). The resulting mixture was stirred at 90° C. overnight under a nitrogen atmosphere. After filtration, the filtrate was concentrated under reduced pressure. This resulted in N-[2,5-difluoro-3-(4,4,5,5-tetramethyl-1,3,2-dioxaborolan-2-yl)phenyl]propane-1-sulfonamide (700 mg, crude) as a black oil. LCMS: C₁₅H₂₂BF₂NO₄S requires: 361.2, found: m/z=362.2 [M+H]⁺.

### Step-4: Synthesis of tert-butyl 4-(4-{4-[2,5-difluoro-3-(propane-1-sulfonamido)phenyl]-3-(pyridin-4-yl)pyrazol-1-yl}phenyl)piperazine-1-carboxylate (5)

To a mixture of N-[2,5-difluoro-3-(4,4,5,5-tetramethyl-1,3,2-dioxaborolan-2-yl)phenyl]propane-1-sulfonamide (700 mg, 1.938 mmol, 3 equiv) and tert-butyl 4-{4-[4-bromo-3-(pyridin-4-yl)pyrazol-1-yl]phenyl}piperazine-1-carboxylate (313 mg, 0.646 mmol, 1 equiv) in dioxane (10 mL) was added Pd(AMPHOS)₂Cl₂ (46 mg, 0.065 mmol, 0.1 equiv) and CsF (196 mg, 1.292 mmol, 2 equiv) in H₂O (2 mL). The resulting mixture was stirred at 90° C. for one hour under a nitrogen atmosphere. The resulting mixture was then concentrated under reduced pressure. The residue was purified by silica gel column chromatography eluted with PE:EtOAc (1:1) to afford tert-butyl 4-(4-{4-[2,5-difluoro-3-(propane-1-sulfonamido)phenyl]-3-(pyridin-4-yl)pyrazol-1-yl}phenyl)piperazine-1-carboxylate (200 mg, 43.61%) as a yellow solid. LCMS: C₃₂H₃₆F₂N₆O₄S requires: 638.2, found: m/z=639.2 [M+H]⁺.

### Step-5: Synthesis of N-(2,5-difluoro-3-{1-[4-(piperazin-1-yl)phenyl]-3-(pyridin-4-yl)pyrazol-4-yl}phenyl)propane-1-sulfonamide Trifluoroacetic Acid Salt (6)

To a mixture of tert-butyl 4-(4-{4-[2,5-difluoro-3-(propane-1-sulfonamido)phenyl]-3-(pyridin-4-yl)pyrazol-1-yl}phenyl)piperazine-1-carboxylate (200 mg, 0.313 mmol, 1 equiv) in DCM (6 mL) was added TFA (2 mL) at 0° C. The resulting mixture was stirred at room temperature for one hour. The resulting mixture was then concentrated under reduced pressure and subjected to lyophilization. This resulted in N-(2,5-difluoro-3-{1-[4-(piperazin-1-yl)phenyl]-3-(pyridin-4-yl)pyrazol-4-yl}phenyl)propane-1-sulfonamide trifluoroacetic acid salt (196.1 mg, crude) as a yellow solid. LCMS: C₂₇H₂₈F₂N₆O₂S requires: 538.2, found: m/z=539.2 [M+H]⁺. ¹H NMR (400 MHz, Methanol-d₄) δ 8.77-8.71 (m, 2H), 8.59 (s, 1H), 8.16-8.09 (m, 2H), 7.94-7.85 (m, 2H), 7.46-7.37 (m, 1H), 7.28-7.20 (m, 2H), 7.19-7.11 (m, 1H), 3.57-3.50 (m, 4H), 3.50-3.40 (m, 4H), 3.22-3.13 (m, 2H), 1.92-1.78 (m, 2H), 1.09-1.01 (m, 3H).

### Synthesis of [(5-chloro-2-fluoro-3-{1-[4-(piperazin-1-yl)phenyl]-3-(pyridin-4-yl)pyrazol-4-yl}phenyl)sulfamoyl]-N-ethyl-N-methylamine Trifluoroacetic Acid Salt (3)

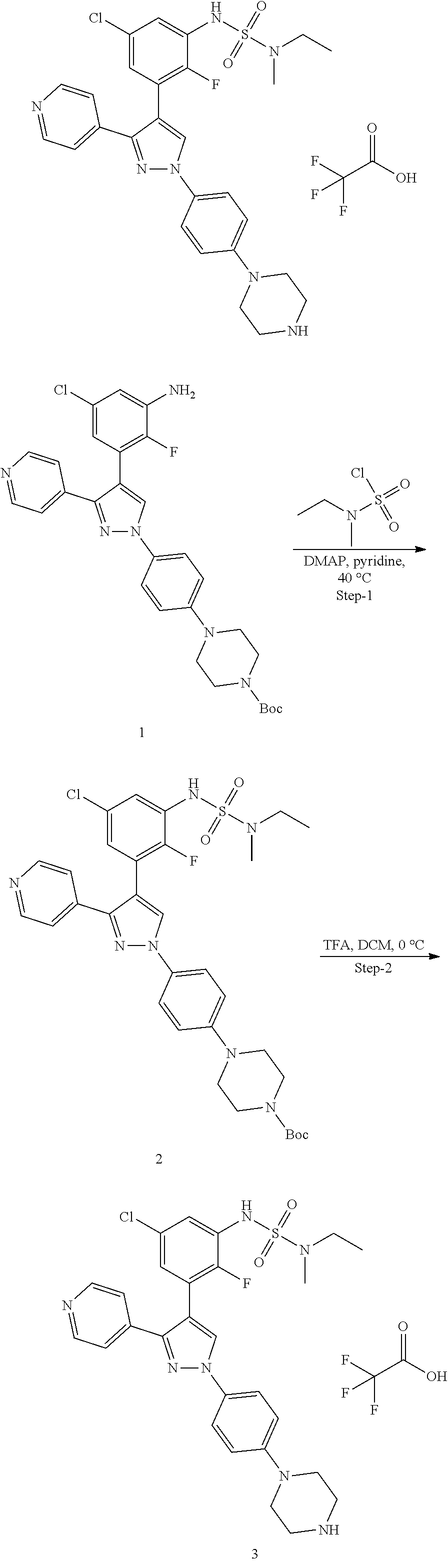

### Step-1: Synthesis of tert-butyl 4-{4-[4-(5-chloro-3-{[ethyl(methyl)sulfamoyl]amino}-2-fluorophenyl)-3-(pyridin-4-yl)pyrazol-1-yl]phenyl}piperazine-1-carboxylate (2)

To a mixture of tert-butyl 4-{4-[4-(3-amino-5-chloro-2-fluorophenyl)-3-(pyridin-4-yl)pyrazol-1-yl]phenyl}piperazine-1-carboxylate (400 mg, 0.729 mmol, 1 equiv) and N-ethyl-N-methylsulfamoyl chloride (230 mg, 1.458 mmol, 2 equiv) in pyridine (1.6 mL) was added DMAP (107 mg, 0.875 mmol, 1.2 equiv). The resulting mixture was stirred at 40° C. overnight under a nitrogen atmosphere. The residue was purified via C18 reverse phase column chromatography with ACN:H₂O (4:1). The residue was then purified by silica gel column chromatography eluted with PE:EtOAc (1:1) to afford tert-butyl 4-{4-[4-(5-chloro-3-{[ethyl(methyl)sulfamoyl]amino}-2-fluorophenyl)-3-(pyridin-4-yl)pyrazol-1-yl]phenyl}piperazine-1-carboxylate (100 mg, 18.43%) as a white solid. LCMS: C₃₂H₃₇ClFN₇O₄S requires: 669.2, found: m/z=670.1 [M+H]⁺.

### Step-2: Synthesis of [(5-chloro-2-fluoro-3-{1-[4-(piperazin-1-yl)phenyl]-3-(pyridin-4-yl)pyrazol-4-yl}phenyl)sulfamoyl]-N-ethyl-N-methylamine Trifluoroacetic Acid Salt (3)

To a mixture of tert-butyl 4-{4-[4-(5-chloro-3-{[ethyl(methyl)sulfamoyl]amino}-2-fluorophenyl)-3-(pyridin-4-yl)pyrazol-1-yl]phenyl}piperazine-1-carboxylate (100 mg, 0.149 mmol, 1 equiv) in DCM (3 mL) was added TFA (1 mL) at 0° C. The resulting mixture was stirred at 0° C. for one hour. The resulting mixture was then concentrated under reduced pressure and subjected to lyophilization. This resulted in [(5-chloro-2-fluoro-3-{1-[4-(piperazin-1-yl)phenyl]-3-(pyridin-4-yl)pyrazol-4-yl}phenyl)sulfamoyl]-N-ethyl-N-methylamine trifluoroacetic acid salt (81.8 mg, crude) as a yellow solid. LCMS: C₂₇H₂₉ClFN₇O₂S requires: 569.2, found: m/z=570.3. [M+H]⁺. ¹H NMR (400 MHz, Methanol-d₄) δ 8.76-8.70 (m, 2H), 8.60-8.56 (m, 1H), 8.09-8.03 (m, 2H), 7.94-7.85 (m, 2H), 7.63-7.56 (m, 1H), 7.38-7.32 (m, 1H), 7.28-7.19 (m, 2H), 3.57-3.50 (m, 4H), 3.47-3.40 (m, 4H), 3.32-3.22 (m, 2H), 2.86-2.82 (m, 3H), 1.20-1.12 (m, 3H).

### Synthesis of N-(5-chloro-2-fluoro-3-{1-[2-fluoro-4-(piperazin-1-yl)phenyl]-3-(pyridin-4-yl)pyrazol-4-yl}phenyl)pyrrolidine-1-sulfonamide Trifluoroacetic Acid Salt

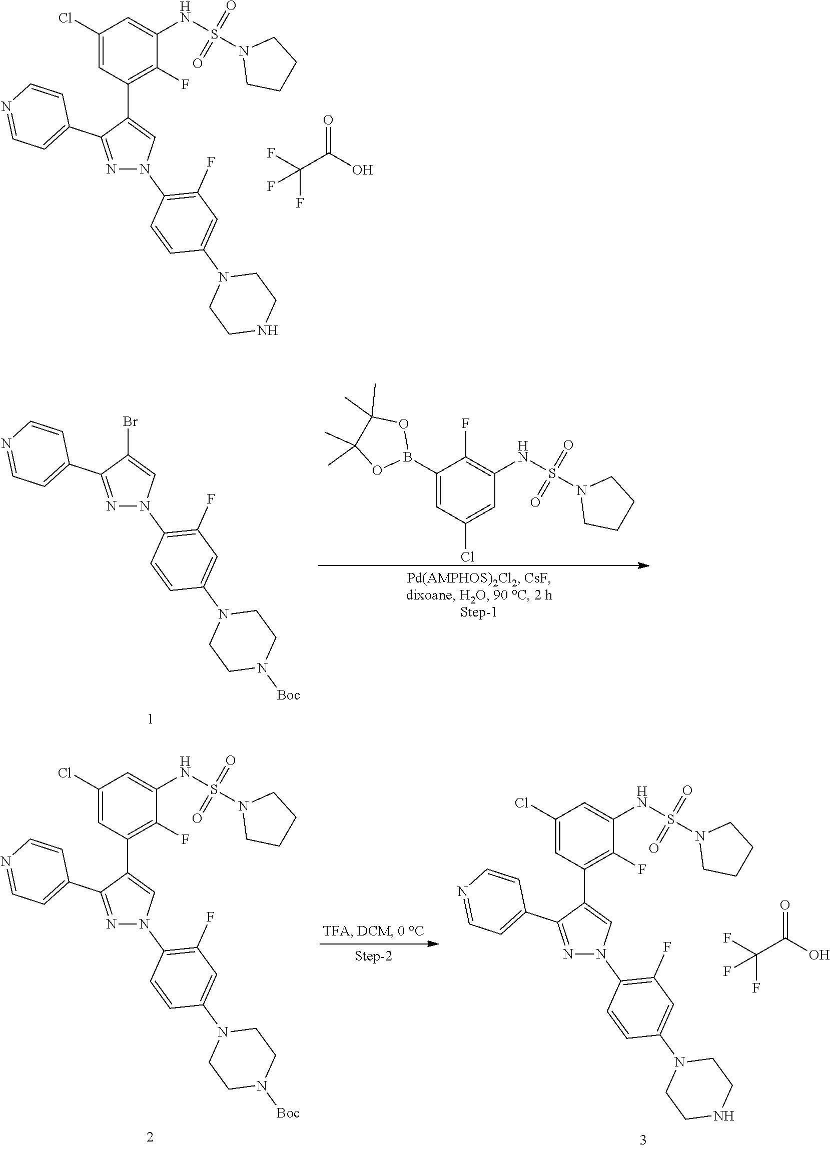

### Step-1: Synthesis of tert-butyl 4-[4-(4-{5-chloro-2-fluoro-3-[(pyrrolidine-1-sulfonyl)amino]phenyl}-3-(pyridin-4-yl)pyrazol-1-yl)-3-fluorophenyl]piperazine-1-carboxylate (2)

To a mixture of tert-butyl 4-{4-[4-bromo-3-(pyridin-4-yl)pyrazol-1-yl]-3-fluorophenyl}piperazine-1-carboxylate (400 mg, 0.796 mmol, 1 equiv) and N-[5-chloro-2-fluoro-3-(4,4,5,5-tetramethyl-1,3,2-dioxaborolan-2-yl)phenyl]pyrrolidine-1-sulfonamide (967 mg, 2.388 mmol, 3 equiv) in dioxane (10 mL) was added CsF (242 mg, 1.592 mmol, 2 equiv) in H₂O (2 mL) and Pd(AMPHOS)₂Cl₂ (59 mg, 0.08 mmol, 0.1 equiv). The resulting mixture was stirred at 90° C. for 2 h under a nitrogen atmosphere. The resulting mixture was then concentrated under vacuum. The residue was purified by silica gel column chromatography eluted with PE:EtOAc (1:10) to afford tert-butyl 4-[4-(4-{5-chloro-2-fluoro-3-[(pyrrolidine-1-sulfonyl)amino]phenyl}-3-(pyridin-4-yl)pyrazol-1-yl)-3-fluorophenyl]piperazine-1-carboxylate (260 mg, 46.64%) as a brown solid. LCMS: C₃₃H₃₆ClF₂N₇O₄S requires: 699.2, found: m/z=700.2 [M+H]⁺.

### Step-2: Synthesis of N-(5-chloro-2-fluoro-3-{1-[2-fluoro-4-(piperazin-1-yl)phenyl]-3-(pyridin-4-yl)pyrazol-4-yl}phenyl)pyrrolidine-1-sulfonamide Trifluoroacetic Acid Salt (3)

To a mixture of tert-butyl 4-[4-(4-{5-chloro-2-fluoro-3-[(pyrrolidine-1-sulfonyl)amino]phenyl}-3-(pyridin-4-yl)pyrazol-1-yl)-3-fluorophenyl]piperazine-1-carboxylate (210 mg, 0.300 mmol, 1 equiv) in DCM (9 mL) was added TFA (3 mL) at 0° C. The resulting mixture was stirred at 0° C. for one hour. The resulting mixture was then concentrated under vacuum. The residue was purified by reverse phase flash chromatography eluted with ACN:H₂O (1:1) to afford N-(5-chloro-2-fluoro-3-{1-[2-fluoro-4-(piperazin-1-yl)phenyl]-3-(pyridin-4-yl)pyrazol-4-yl}phenyl)pyrrolidine-1-sulfonamide trifluoroacetic acid salt (137.6 mg, 61.48%) as a yellow solid. LCMS: C₂₈H₂₈ClF₂N₇O₂S requires: 599.2, found: m/z=600.2 [M+H]⁺. ¹H NMR (400 MHz, Methanol-d₄) δ 8.70 (s, 2H), 8.36 (s, 1H), 7.99-7.93 (m, 2H), 7.84 (s, 1H), 7.63 (s, 1H), 7.30 (s, 1H), 7.14-7.04 (m, 2H), 3.59 (s, 4H), 3.43 (s, 4H), 3.34-3.28 (m, 4H), 1.96-1.86 (m, 4H).

### Synthesis of N-(5-chloro-2-fluoro-3-{1-[5-(piperazin-1-yl)pyridin-2-yl]-3-(pyridin-4-yl)pyrazol-4-yl}phenyl)propane-1-sulfonamide Trifluoroacetic Acid Salt (6)

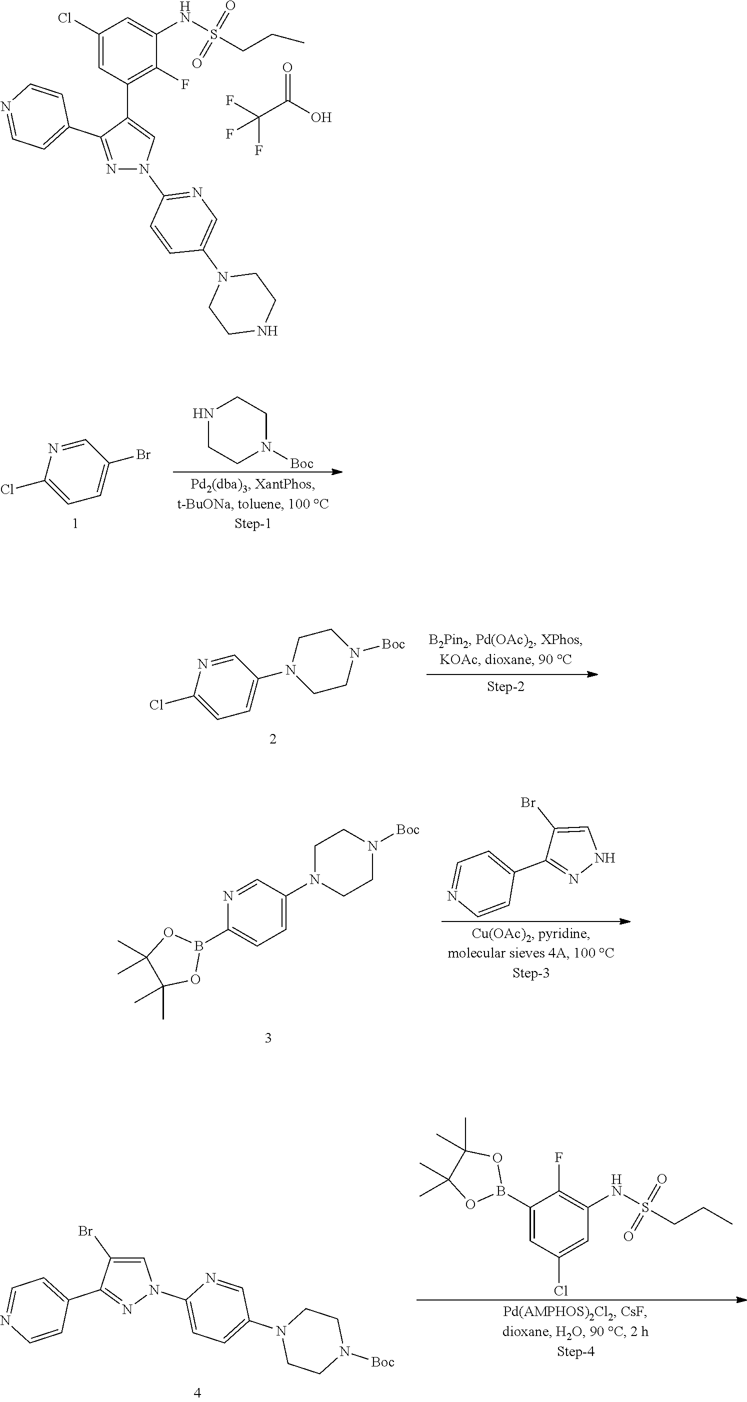

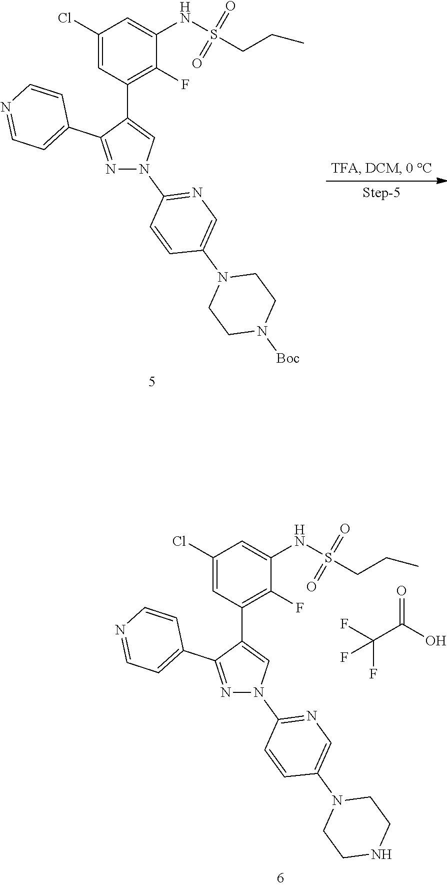

### Step-1: Synthesis of tert-butyl 4-(6-chloropyridin-3-yl)piperazine-1-carboxylate (2)

To a mixture of 5-bromo-2-chloropyridine (5 g, 25.982 mmol, 1 equiv) and tert-butyl piperazine-1-carboxylate (4.8 g, 25.982 mmol, 1 equiv) in toluene (150 mL) was added XantPhos (902 mg, 1.559 mmol, 0.06 equiv), Pd₂(dba)₃ (476 mg, 0.520 mmol, 0.02 equiv), and t-BuOK (3.8 g, 38.973 mmol, 1.5 equiv). The resulting mixture was stirred at 100° C. overnight under a nitrogen atmosphere. The resulting mixture was then extracted with EtOAc. The combined organic layers were washed with water and dried over anhydrous Na₂SO₄. After filtration, the filtrate was concentrated under reduced pressure. The residue was purified by silica gel column chromatography eluted with PE:EtOAc (3:1) to afford tert-butyl 4-(6-chloropyridin-3-yl)piperazine-1-carboxylate (7 g, 81.43%) as a white solid. LCMS: C₁₄H₂₀C1N₃O₂ requires: 297.1, found: m/z=298.2 [M+H]⁺.

### Step-2: Synthesis of tert-butyl 4-[6-(4,4,5,5-tetramethyl-1,3,2-dioxaborolan-2-yl)pyridin-3-yl]piperazine-1-carboxylate (3)

To a mixture of tert-butyl 4-(6-chloropyridin-3-yl)piperazine-1-carboxylate (2 g, 6.716 mmol, 1 equiv) and 4,4,5,5-tetramethyl-2-(4,4,5,5-tetramethyl-1,3,2-dioxaborolan-2-yl)-1,3,2-dioxaborolane (B₂Pin₂) (1.9 g, 7.388 mmol, 1.1 equiv) in 1,4-dioxane (40 mL) was added Pd(OAc)₂ (181 mg, 0.806 mmol, 0.12 equiv), XPhos (576 mg, 1.209 mmol, 0.18 equiv), and KOAc (2 g, 20.148 mmol, 3 equiv). The resulting mixture was stirred at 90° C. overnight under a nitrogen atmosphere. The resulting mixture was then concentrated under reduced pressure. After filtration, the filtrate was concentrated under reduced pressure. This resulted in tert-butyl 4-[6-(4,4,5,5-tetramethyl-1,3,2-dioxaborolan-2-yl)pyridin-3-yl]piperazine-1-carboxylate (3 g, crude) as a black oil. LCMS: C₂₀H₃₂BN₃O₄ requires: 389.3, found: m/z=390.2 [M+H]⁺.

### Step-3: Synthesis of tert-butyl 4-{6-[4-bromo-3-(pyridin-4-yl)pyrazol-1-yl]pyridin-3-yl}piperazine-1-carboxylate (4)

To a mixture of tert-butyl 4-[6-(4,4,5,5-tetramethyl-1,3,2-dioxaborolan-2-yl)pyridin-3-yl]piperazine-1-carboxylate (3 g, 7.706 mmol, 1 equiv) and 4-(4-bromo-1H-pyrazol-3-yl)pyridine (2.4 g, 10.801 mmol, 1.4 equiv) in pyridine (30 mL) was added Cu(OAc)₂ (2.8 g, 15.412 mmol, 2 equiv) and molecular sieves (4A) (2.4 g). The resulting mixture was stirred at 100° C. overnight under an oxygen atmosphere. The resulting mixture was then concentrated under reduced pressure. The residue was purified by silica gel column chromatography eluted with PE:EtOAc (1:1) to afford tert-butyl 4-{6-[4-bromo-3-(pyridin-4-yl)pyrazol-1-yl]pyridin-3-yl}piperazine-1-carboxylate (230 mg, 5.23%) as a yellow solid. LCMS: C₂₂H₂₅BrN₆O₂ requires: 484.1, found: m/z=485.1 [M+H]⁺.

### Step-4: Synthesis of tert-butyl 4-(6-{4-[5-chloro-2-fluoro-3-(propane-1-sulfonamido)phenyl]-3-(pyridin-4-yl)pyrazol-1-yl}pyridin-3-yl)piperazine-1-carboxylate

To a mixture of tert-butyl 4-{6-[4-bromo-3-(pyridin-4-yl)pyrazol-1-yl]pyridin-3-yl}piperazine-1-carboxylate (220 mg, 0.453 mmol, 1 equiv) and N-[5-chloro-2-fluoro-3-(4,4,5,5-tetramethyl-1,3,2-dioxaborolan-2-yl)phenyl]propane-1-sulfonamide (514 mg, 1.359 mmol, 3 equiv) in dioxane (5 mL) was added Pd(AMPHOS)₂Cl₂ (32 mg, 0.045 mmol, 0.1 equiv) and CsF (138 mg, 0.906 mmol, 2 equiv) in H₂O (1 mL). The resulting mixture was stirred at 90° C. for 2 h under a nitrogen atmosphere. The resulting mixture was then concentrated under reduced pressure. The residue was purified by silica gel column chromatography eluted with PE:EtOAc (1:1) to afford tert-butyl 4-(6-{4-[5-chloro-2-fluoro-3-(propane-1-sulfonamido)phenyl]-3-(pyridin-4-yl)pyrazol-1-yl}pyridin-3-yl)piperazine-1-carboxylate (150 mg, 45.39%) as a white solid. LCMS: C₃1H₃₅ClFN₇O₄S: 655.2, found: m/z=656.2 [M+H]⁺.

### Step-5: Synthesis of N-(5-chloro-2-fluoro-3-{1-[5-(piperazin-1-yl)pyridin-2-yl]-3-(pyridin-4-yl)pyrazol-4-yl}phenyl)propane-1-sulfonamide Trifluoroacetic Acid Salt

To a mixture of tert-butyl 4-(6-{4-[5-chloro-2-fluoro-3-(propane-1-sulfonamido)phenyl]-3-(pyridin-4-yl)pyrazol-1-yl}pyridin-3-yl)piperazine-1-carboxylate (145 mg, 0.221 mmol, 1 equiv) in DCM (6 mL) was added TFA (2 mL) at 0° C. The resulting mixture was stirred at room temperature for one hour. The resulting mixture was then concentrated under reduced pressure and subjected to lyophilization. This resulted in N-(5-chloro-2-fluoro-3-{1-[5-(piperazin-1-yl)pyridin-2-yl]-3-(pyridin-4-yl)pyrazol-4-yl}phenyl)propane-1-sulfonamide trifluoroacetic acid salt (125.4 mg, crude) as a yellow solid. LCMS: C₂₆H₂₇ClFN₇O₂S requires: 555.2, found: m/z=556.2 [M+H]⁺. ¹H NMR (400 MHz, Methanol-d₄) δ 8.85 (s, 1H), 8.76-8.70 (m, 2H), 8.35-8.24 (m, 1H), 8.14-8.03 (m, 3H), 7.81-7.69 (m, 1H), 7.67-7.58 (m, 1H), 7.44-7.31 (m, 1H), 3.63-3.54 (m, 4H), 3.51-3.43 (m, 4H), 3.20-3.12 (m, 2H), 1.91-1.78 (m, 2H), 1.09-1.00 (m, 3H).

### Synthesis of N-(5-chloro-2-fluoro-3-{1-[4-(piperazin-1-yl)phenyl]-3-(pyridin-4-yl)pyrazol-4-yl}phenyl)pyrrolidine-1-sulfonamide Trifluoroacetic Acid Salt (6)

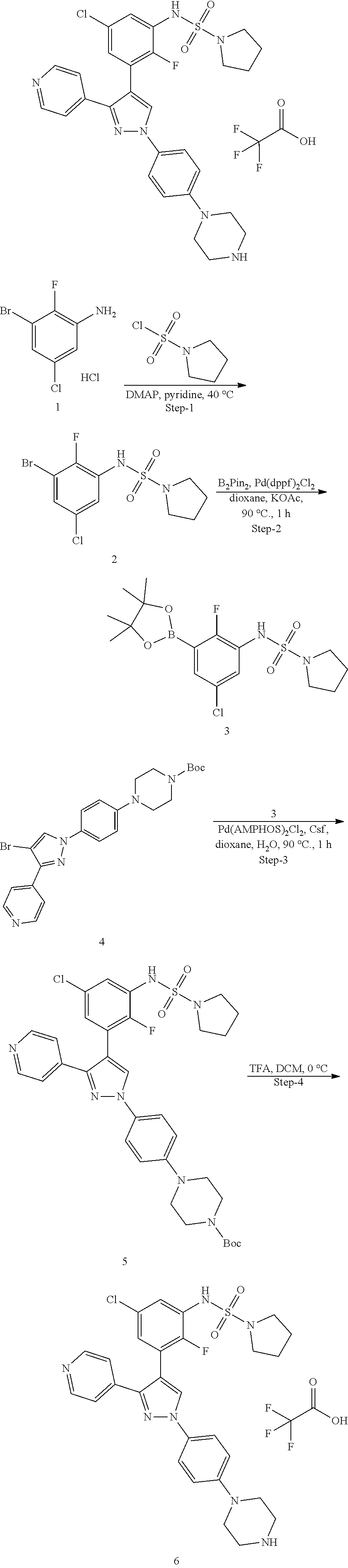

### Step-1: Synthesis of N-(3-bromo-5-chloro-2-fluorophenyl)pyrrolidine-1-sulfonamide (2)

To a mixture of 3-bromo-5-chloro-2-fluoroaniline hydrochloride (2 g, 7.665 mmol, 1 equiv) and pyrrolidine-1-sulfonyl chloride (2.6 g, 15.33 mmol, 2 equiv) in pyridine (4 mL) was added DMAP (1.12 g, 9.198 mmol, 1.2 equiv). The resulting mixture was stirred at 40° C. overnight under a nitrogen atmosphere. The resulting mixture was then concentrated under reduced pressure. The residue was purified by silica gel column chromatography eluted with PE:EtOAc (3:1) to afford N-(3-bromo-5-chloro-2-fluorophenyl)pyrrolidine-1-sulfonamide (2 g, 65.66%) as a yellow solid. LCMS: C₁₀H₁₁BrClFN₂O₂S requires: 355.9, found: m/z=357.1 [M+H]⁺.

### Step-2: Synthesis of N-[5-chloro-2-fluoro-3-(4,4,5,5-tetramethyl-1,3,2-dioxaborolan-2-yl)phenyl]pyrrolidine-1-sulfonamide (3)

To a mixture of N-(3-bromo-5-chloro-2-fluorophenyl)pyrrolidine-1-sulfonamide (2 g, 5.593 mmol, 1 equiv) and bis(pinacolato)diboron (B₂Pin₂) (2.84 g, 11.186 mmol, 2 equiv) in dioxane (30 mL) was added KOAc (1.1 g, 11.186 mmol, 2 equiv) and Pd(dppf)Cl₂ (0.41 g, 0.559 mmol, 0.1 equiv). The resulting mixture was stirred at 90° C. for 1 h under a nitrogen atmosphere. The resulting mixture was then filtered. The filtrate was concentrated under reduced pressure. This resulted in N-[5-chloro-2-fluoro-3-(4,4,5,5-tetramethyl-1,3,2-dioxaborolan-2-yl)phenyl]pyrrolidine-1-sulfonamide (2 g, crude) as a black oil. LCMS: C₁₆H₂₃BClFN₂O₄S requires: 404.1, found: m/z=405.2 [M+H]⁺.

### Step-3: Synthesis of tert-butyl 4-[4-(4-{5-chloro-2-fluoro-3-[(pyrrolidine-1-sulfonyl)amino]phenyl}-3-(pyridin-4-yl)pyrazol-1-yl)phenyl]piperazine-1-carboxylate

To a mixture of tert-butyl 4-{4-[4-bromo-3-(pyridin-4-yl)pyrazol-1-yl]phenyl}piperazine-1-carboxylate (800 mg, 1.652 mmol, 1 equiv) and N-[5-chloro-2-fluoro-3-(4,4,5,5-tetramethyl-1,3,2-dioxaborolan-2-yl)phenyl]pyrrolidine-1-sulfonamide (2 g, 4.956 mmol, 3 equiv) in dioxane (20 mL) was added CsF (502 mg, 3.304 mmol, 2 equiv) in H₂O (4 mL) and Pd(AMPHOS)₂Cl₂ (117 mg, 0.165 mmol, 0.1 equiv). The resulting mixture was stirred at 90° C. for 1 h under a nitrogen atmosphere. The resulting mixture was then concentrated under vacuum. The residue was purified by silica gel column chromatography eluted with PE:EtOAc (1:2) to afford tert-butyl 4-[4-(4-{5-chloro-2-fluoro-3-[(pyrrolidine-1-sulfonyl)amino]phenyl}-3-(pyridin-4-yl)pyrazol-1-yl)phenyl]piperazine-1-carboxylate (350 mg, 26.40%) as an off-white solid. LCMS: C₃₃H₃₇ClFN₇O₄S requires: 681.2, found: m/z=682.2 [M+H]⁺.

### Step-4: Synthesis of N-(5-chloro-2-fluoro-3-{1-[4-(piperazin-1-yl)phenyl]-3-(pyridin-4-yl)pyrazol-4-yl}phenyl)pyrrolidine-1-sulfonamide Trifluoroacetic Acid Salt

To a mixture of tert-butyl 4-[4-(4-{5-chloro-2-fluoro-3-[(pyrrolidine-1-sulfonyl)amino]phenyl}-3-(pyridin-4-yl)pyrazol-1-yl)phenyl]piperazine-1-carboxylate (360 mg, 0.528 mmol, 1 equiv) in DCM (9 mL) was added TFA (3 mL) at 0° C. The resulting mixture was stirred at 0° C. for one hour. The resulting mixture was then concentrated under vacuum. The residue was purified by reverse phase flash chromatography eluted with ACN:H₂O (1:1) to afford N-(5-chloro-2-fluoro-3-{1-[4-(piperazin-1-yl)phenyl]-3-(pyridin-4-yl)pyrazol-4-yl}phenyl)pyrrolidine-1-sulfonamide trifluoroacetic acid salt (161.0 mg, 61.45%) as a yellow solid. LCMS: C₂₈H₂₉ClFN₇O₂S requires: 581.2, found: m/z=582.2 [M+H]⁺. ¹H NMR (400 MHz, Methanol-d₄) δ 8.78-8.72 (m, 2H), 8.59 (s, 1H), 8.15-8.08 (m, 2H), 7.93-7.85 (m, 2H), 7.64 (s, 1H), 7.36 (s, 1H), 7.28-7.19 (m, 2H), 3.54 (s, 4H), 3.44 (s, 4H), 3.33 (s, 2H), 3.35 (s, 2H) 1.98-1.85 (m, 4H).

### Synthesis of N-(5-chloro-2-fluoro-3-{1-[2-fluoro-4-(piperazin-1-yl)phenyl]-3-(pyridin-4-yl)pyrazol-4-yl}phenyl)methanesulfonamide Trifluoroacetic Acid Salt (4)

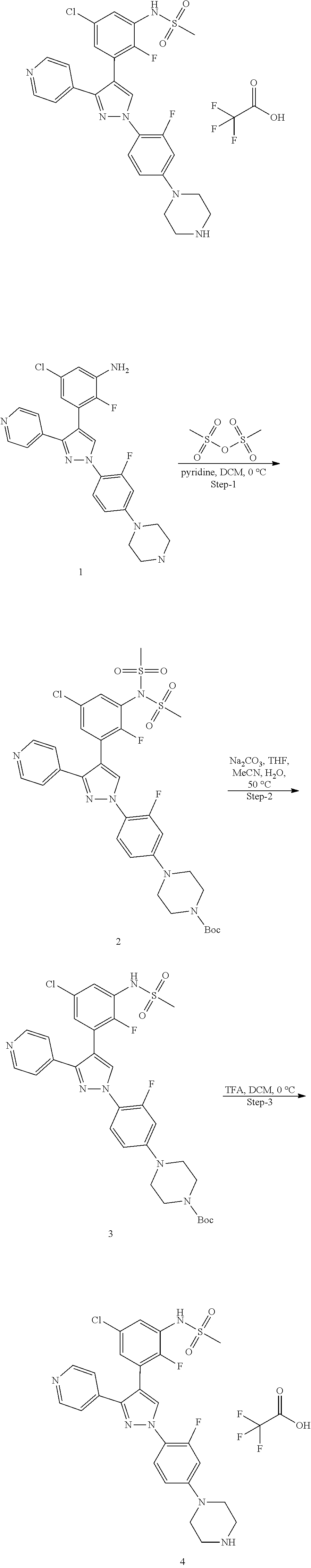

### Step-1 Synthesis of tert-butyl 4(4-{4-[5-chloro-2-fluoro-3-N-methanesulfonylmethanesulfonamido)phenyl]-3-(pyridin-4-yl)pyrazol-1-yl}-3-fluorophenyl)piperazine-1-carboxylate (2)

To a mixture of tert-butyl 4-{4-[4-(3-amino-5-chloro-2-fluorophenyl)-3-(pyridin-4-yl)pyrazol-1-yl]-3-fluorophenyl}piperazine-1-carboxylate (400 mg, 0.705 mmol, 1 equiv) and pyridine (837 mg, 10.575 mmol, 15 equiv) in DCM (10 mL) was added methanesulfonic anhydride (737 mg, 4.23 mmol, 6 equiv) at 0° C. The resulting mixture was stirred at room temperature for one hour. The resulting mixture was then extracted with CH₂Cl₂. The combined organic layers were washed with water and dried over anhydrous Na₂SO₄. After filtration, the filtrate was concentrated under reduced pressure. This resulted in tert-butyl 4-(4-{4-[5-chloro-2-fluoro-3-(N-methanesulfonylmethanesulfonamido)phenyl]-3-(pyridin-4-yl)pyrazol-1-yl}-3-fluorophenyl)piperazine-1-carboxylate (385 mg, crude) as a yellow solid. LCMS: C₃₁H₃₃ClF₂N₆O₆S₂ requires: 722.2, found: m/z=723.2 [M+H]⁺.

### Step-2 Synthesis of tert-butyl 4-{4-[4(5-chloro-2-fluoro-3-methanesulfonamidophenyl)-3-(pyridin-4-yl)pyrazol-1-yl]-3-fluorophenyl}piperazine-1-carboxylate (3)

To a mixture of tert-butyl 4-(4-{4-[5-chloro-2-fluoro-3-(N-methanesulfonylmethanesulfonamido)phenyl]-3-(pyridin-4-yl)pyrazol-1-yl}-3-fluorophenyl)piperazine-1-carboxylate (375 mg, 0.519 mmol, 1 equiv) in THF (5 mL) and MeCN (5 mL) was added Na₂CO₃ (824 mg, 7.785 mmol, 15 equiv) in H₂O (5 mL). The resulting mixture was stirred at 50° C. overnight. The resulting mixture was then concentrated under reduced pressure. The residue was purified by silica gel column chromatography eluted with PE:EtOAc (1:4) to afford tert-butyl 4-{4-[4-(5-chloro-2-fluoro-3-methanesulfonamidophenyl)-3-(pyridin-4-yl)pyrazol-1-yl]-3-fluorophenyl}piperazine-1-carboxylate (250 mg, 74.74%) as an off-white solid. LCMS: C₃₀H₃₁ClF₂N₆O₄S requires: 644.2, found: m/z=645.2 [M+H]⁺.

### Step-3 Synthesis of N-(5-chloro-2-fluoro-3-{1-[2-fluoro-4-(piperazin-1-yl)phenyl]-3-(pyridin-4-yl)pyrazol-4-yl}phenyl)methanesulfonamide Trifluoroacetic Acid Salt (4)

To a mixture of tert-butyl 4-{4-[4-(5-chloro-2-fluoro-3-methanesulfonamidophenyl)-3-(pyridin-4-yl)pyrazol-1-yl]-3-fluorophenyl}piperazine-1-carboxylate (380 mg, 0.589 mmol, 1 equiv) in DCM (9 mL) was added TFA (3 mL) at 0° C. The resulting mixture was stirred at room temperature for one hour. The resulting mixture was then concentrated under reduced pressure and subjected to lyophilization. This resulted in N-(5-chloro-2-fluoro-3-{1-[2-fluoro-4-(piperazin-1-yl)phenyl]-3-(pyridin-4-yl)pyrazol-4-yl}phenyl)methanesulfonamide trifluoroacetic acid salt (390.8 mg, crude) as a yellow solid. LCMS: C₂₅H₂₃ClF₂N₆O₂S requires: 544.1, found: m/z=545.2 [M+H]⁺. ¹H NMR (400 MHz, Methanol-d₄) δ 8.77-8.71 (m, 2H), 8.40 (s, 1H), 8.11-8.05 (m, 2H), 7.85 (s, 1H), 7.62 (s, 1H), 7.40 (s, 1H), 7.15-7.02 (m, 2H), 3.59 (s, 4H), 3.43 (s, 4H), 3.08 (s, 3H).

### Synthesis of N-(5-chloro-2-fluoro-3-{1-[5-(piperazin-1-yl)pyridin-2-yl]-3-(pyridin-4-yl)pyrazol-4-yl}phenyl)pyrrolidine-1-sulfonamide Trifluoroacetic Acid Salt (3)

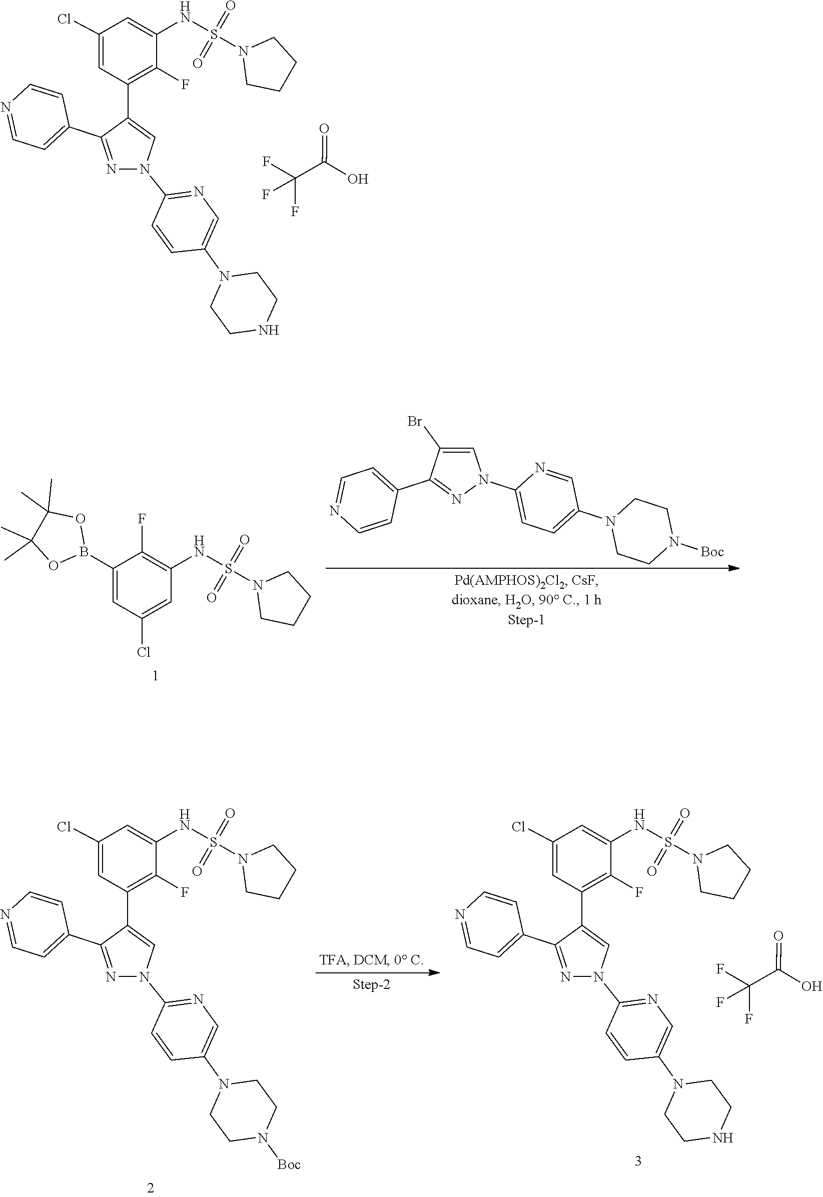

### Step-1: Synthesis of tert-butyl 4-[6-(4-{5-chloro-2-fluoro-3-[(pyrrolidine-1-sulfonyl)amino]phenyl}-3-(pyridin-4-yl)pyrazol-1-yl)pyridin-3-yl]piperazine-1-carboxylate (2)

To a mixture of N-[5-chloro-2-fluoro-3-(4,4,5,5-tetramethyl-1,3,2-dioxaborolan-2-yl)phenyl]pyrrolidine-1-sulfonamide (1 g, 2.472 mmol, 3 equiv) and tert-butyl 4-{6-[4-bromo-3-(pyridin-4-yl)pyrazol-1-yl]pyridin-3-yl}piperazine-1-carboxylate (400 mg, 0.824 mmol, 1 equiv) in dioxane (10 mL) was added CsF (83.87 mg, 0.552 mmol, 0.67 equiv) in H₂O (2 mL) and Pd(AMPHOS)₂Cl₂ (58.35 mg, 0.082 mmol, 0.1 equiv). The resulting mixture was stirred at 90° C. for 1 h under a nitrogen atmosphere. The resulting mixture was then concentrated under vacuum. The residue was purified by silica gel column chromatography eluted with PE:EtOAc (1:1) to afford tert-butyl 4-[6-(4-{5-chloro-2-fluoro-3-[(pyrrolidine-1-sulfonyl)amino]phenyl}-3-(pyridin-4-yl)pyrazol-1-yl)pyridin-3-yl]piperazine-1-carboxylate (600 mg, 35.52%) as a yellow solid. LCMS: C₃₂H₃₆ClFN₈O₄S requires: 682.2, found: m/z=683.2 [M+H]⁺.

### Step-2: Synthesis of N-(5-chloro-2-fluoro-3-{1-[5-(piperazin-1-yl)pyridin-2-yl]-3-(pyridin-4-yl)pyrazol-4-yl}phenyl)pyrrolidine-1-sulfonamide Trifluoroacetic Acid Salt (3)

To a mixture of tert-butyl 4-[6-(4-{5-chloro-2-fluoro-3-[(pyrrolidine-1-sulfonyl)amino]phenyl}-3-(pyridin-4-yl)pyrazol-1-yl)pyridin-3-yl]piperazine-1-carboxylate (590 mg, 0.864 mmol, 1 equiv) in DCM (9 mL) was added TFA (3 mL) at 0° C. The resulting mixture was stirred at 0° C. for one hour. The resulting mixture was then concentrated under vacuum. The residue was purified by reverse phase flash chromatography eluted with ACN:H₂O (1:1) to afford N-(5-chloro-2-fluoro-3-{1-[5-(piperazin-1-yl)pyridin-2-yl]-3-(pyridin-4-yl)pyrazol-4-yl}phenyl)pyrrolidine-1-sulfonamide trifluoroacetic acid salt (509.1 mg, 84.06%) as a yellow solid. LCMS: C₂₇H₂₈ClFN₈O₂S requires: 582.1, found: m/z=583.2 [M+H]⁺. ¹H NMR (400 MHz, Methanol-d₄) δ 8.84 (s, 1H), 8.77-8.71 (m, 2H), 8.29 (s, 1H), 8.10-8.02 (m, 3H), 7.73 (s, 1H), 7.64 (s, 1H), 7.32 (s, 1H), 3.76 (s, 4H), 3.59 (s, 4H), 3.46 (s, 2H), 3.33 (s, 2H), 1.98-1.85 (m, 4H).

### Synthesis of N-(2,5-difluoro-3-{1-[4-(piperazin-1-yl)phenyl]-3-(pyridin-4-yl)pyrazol-4-yl}phenyl)pyrrolidine-1-sulfonamide Trifluoroacetic Acid Salt (5)

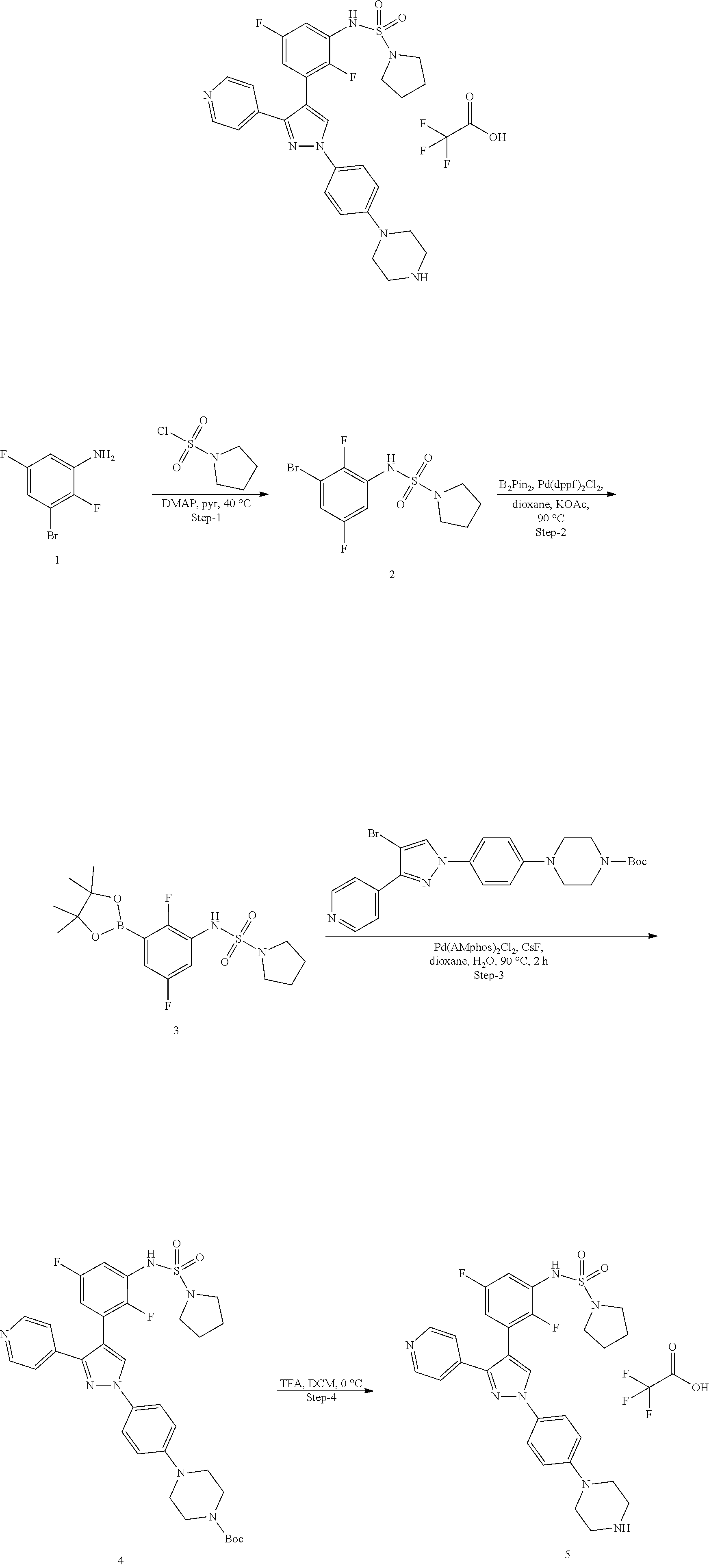

### Step-1: Synthesis of N-(3-bromo-2,5-difluorophenyl)pyrrolidine-1-sulfonamide (2)

To a mixture of 3-bromo-2,5-difluoroaniline (2.5 g, 12.019 mmol, 1 equiv) and pyrrolidine-1-sulfonyl chloride (4.08 g, 24.038 mmol, 2 equiv) in pyridine (4 mL) was added DMAP (1.76 g, 14.423 mmol, 1.2 equiv). The resulting mixture was stirred at 40° C. for 2 days under a nitrogen atmosphere. The resulting mixture was then concentrated under reduced pressure. The residue was purified by silica gel column chromatography eluted with PE:EtOAc (5:1) to afford N-(3-bromo-2,5-difluorophenyl)pyrrolidine-1-sulfonamide (2.8 g, 54.63%) as a white solid. LCMS: C₁₀H₁₁BrF₂N₂O₂S requires: 340.0, found: m/z=341.1 [M+H]⁺.

### Step-2: Synthesis of N-[2,5-difluoro-3-(4,4,5,5-tetramethyl-1,3,2-dioxaborolan-2-yl)phenyl]pyrrolidine-1-sulfonamide (3)

To a mixture of N-(3-bromo-2,5-difluorophenyl)pyrrolidine-1-sulfonamide (500 mg, 1.466 mmol, 1 equiv) and 4,4,5,5-tetramethyl-2-(4,4,5,5-tetramethyl-1,3,2-dioxaborolan-2-yl)-1,3,2-dioxaborolane (B₂Pin₂) (744 mg, 2.932 mmol, 2 equiv) in 1,4-dioxane (10 mL) was added Pd(dppf)Cl₂ (120 mg, 0.147 mmol, 0.1 equiv) and KOAc (288 mg, 2.932 mmol, 2 equiv). The resulting mixture was stirred at 90° C. overnight under a nitrogen atmosphere. The resulting mixture was then filtered. The filtrate was concentrated under reduced pressure. This resulted in N-[2,5-difluoro-3-(4,4,5,5-tetramethyl-1,3,2-dioxaborolan-2-yl)phenyl]pyrrolidine-1-sulfonamide (500 mg, 87.88%) as a black oil. LCMS: C₁₆H₂₃BF₂N₂O₄S requires: 388.1, found: m/z=389.2 [M+H]⁺.

### Step-3: Synthesis of tert-butyl 4-[4-(4-{2,5-difluoro-3-[(pyrrolidine-1-sulfonyl)amino]phenyl}-3-(pyridin-4-yl)pyrazol-1-yl)phenyl]piperazine-1-carboxylate (4)

To a mixture of N-[2,5-difluoro-3-(4,4,5,5-tetramethyl-1,3,2-dioxaborolan-2-yl)phenyl]pyrrolidine-1-sulfonamide (500 mg, 1.29 mmol, 1 equiv) and tert-butyl 4-{4-[4-bromo-3-(pyridin-4-yl)pyrazol-1-yl]phenyl}piperazine-1-carboxylate (625 mg, 1.29 mmol, 1 equiv) in dioxane (10 mL) was added Pd(AMPHOS)₂Cl₂ (91 mg, 0.129 mmol, 0.1 equiv) and CsF (392 mg, 2.58 mmol, 2 equiv) in H₂O (2 mL). The resulting mixture was stirred at 90° C. for 2 h under a nitrogen atmosphere. The resulting mixture was then concentrated under reduced pressure. The residue was purified by silica gel column chromatography eluted with PE:EtOAc (1:4) to afford tert-butyl 4-[4-(4-{2,5-difluoro-3-[(pyrrolidine-1-sulfonyl)amino]phenyl}-3-(pyridin-4-yl)pyrazol-1-yl)phenyl]piperazine-1-carboxylate (400 mg, 40.51%) as an off-white solid. LCMS: C₃₃H₃₇F₂N₇O₄S requires: 665.3, found: m/z=666.3 [M+H]⁺.

### Step-4: Synthesis of N-(2,5-difluoro-3-{1-[4-(piperazin-1-yl)phenyl]-3-(pyridin-4-yl)pyrazol-4-yl}phenyl)pyrrolidine-1-sulfonamide Trifluoroacetic Acid Salt (5)

To a mixture of tert-butyl 4-[4-(4-{2,5-difluoro-3-[(pyrrolidine-1-sulfonyl)amino]phenyl}-3-(pyridin-4-yl)pyrazol-1-yl)phenyl]piperazine-1-carboxylate (400 mg, 0.601 mmol, 1 equiv) in DCM (9 mL) was added TFA (3 mL) at 0° C. The resulting mixture was stirred at room temperature for one hour. The resulting mixture was then concentrated under reduced pressure. The residue was purified by reverse phase flash chromatography eluted with ACN:H₂O (1:4) to afford N-(2,5-difluoro-3-{1-[4-(piperazin-1-yl)phenyl]-3-(pyridin-4-yl)pyrazol-4-yl}phenyl)pyrrolidine-1-sulfonamide trifluoroacetic acid salt (212.6 mg, 51.70%) as a yellow solid. LCMS: C₂₈H₂₉F₂N₇O₂S requires: 565.2, found: m/z=566.3 [M+H]⁺. ¹H NMR (400 MHz, Methanol-d₄) δ 8.79-8.69 (m, 2H), 8.57 (s, 1H), 8.13-8.03 (m, 2H), 7.95-7.86 (m, 2H), 7.48-7.37 (m, 1H), 7.30-7.20 (m, 2H), 7.14-7.03 (m, 1H), 3.60-3.49 (m, 4H), 3.44 (m, 4H), 3.35 (m, 2H), 3.31 (m, 2H), 2.06-1.80 (m, 4H).

### Synthesis of (3R)—N-(5-chloro-2-fluoro-3-{1-[4-(piperazin-1-yl)phenyl]-3-(pyridin-4-yl)pyrazol-4-yl}phenyl)-3-fluoropyrrolidine-1-sulfonamide Trifluoroacetic Acid Salt (6)

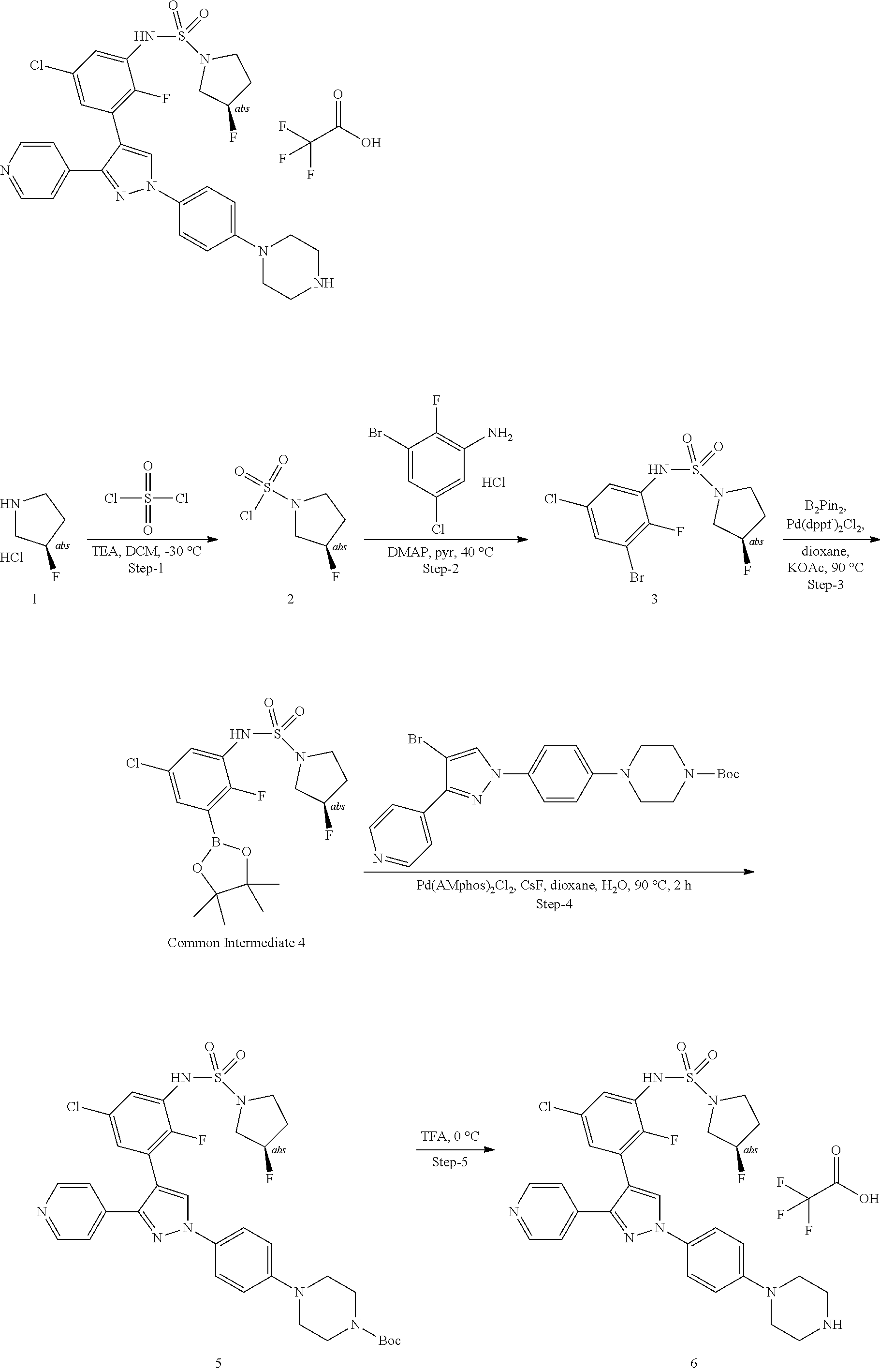

### Step-1: Synthesis of (3R)-3-fluoropyrrolidine-1-sulfonyl Chloride (2)

To a mixture of (3R)-3-fluoropyrrolidine hydrochloride (20 g, 159.274 mmol, 1 equiv) in DCM (400 mL) was added TEA (67 mL, 477.822 mmol, 3 equiv). To the above mixture was then added sulfonyl chloride (26 mL, 318.548 mmol, 2 equiv) in DCM (100 mL) dropwise over 30 min at −30° C. The resulting mixture was stirred at room temperature overnight under a nitrogen atmosphere. The resulting mixture was then diluted with HCl (1N) (300 mL). The resulting mixture was extracted with DCM. The combined organic layers were washed with water and dried over anhydrous Na₂SO₄. After filtration, the filtrate was concentrated under reduced pressure. This resulted in (3R)-3-fluoropyrrolidine-1-sulfonyl chloride (20 g, crude) as a dark yellow solid. LCMS: C₄H₇ClFNO₂S requires: 187.0, found: 188.6.

### Step-2: Synthesis of (3R)—N-(3-bromo-5-chloro-2-fluorophenyl)-3-fluoropyrrolidine-1-sulfonamide (3)

To a mixture of 3-bromo-5-chloro-2-fluoroaniline hydrochloride (5 g, 19.163 mmol, 1 equiv) and (3R)-3-fluoropyrrolidine-1-sulfonyl chloride (7.19 g, 38.326 mmol, 2 equiv) in pyridine (8 mL) was added DMAP (2.81 g, 22.996 mmol, 1.2 equiv). The resulting mixture was stirred at 40° C. for 2 days under a nitrogen atmosphere. The resulting mixture was then concentrated under reduced pressure. The residue was purified by silica gel column chromatography eluted with PE:EtOAc (5:1) to afford (3R)—N-(3-bromo-5-chloro-2-fluorophenyl)-3-fluoropyrrolidine-1-sulfonamide (5 g, 57.66%) as a brown solid. LCMS: C₁₀H₁₀BrClF₂N₂O₂S requires: 373.9, found: m/z=373.1 [M−H]⁻.

### Step-3: Synthesis of (3R)—N-[5-chloro-2-fluoro-3-(4,4,5,5-tetramethyl-1,3,2-dioxaborolan-2-yl)phenyl]-3-fluoropyrrolidine-1-sulfonamide (4)

To a mixture of (3R)—N-(3-bromo-5-chloro-2-fluorophenyl)-3-fluoropyrrolidine-1-sulfonamide (2 g, 5.325 mmol, 1 equiv) and 4,4,5,5-tetramethyl-2-(4,4,5,5-tetramethyl-1,3,2-dioxaborolan-2-yl)-1,3,2-dioxaborolane (B₂Pin₂) (2.7 g, 10.632 mmol, 2 equiv) in 1,4-dioxane (20 mL) was added Pd(dppf)Cl₂ (434 mg, 0.533 mmol, 0.1 equiv) and KOAc (1.05 g, 10.65 mmol, 2 equiv). The resulting mixture was stirred at 90° C. overnight under a nitrogen atmosphere. The resulting mixture was then filtered. The filtrate was concentrated under reduced pressure. This resulted in (3R)—N-[5-chloro-2-fluoro-3-(4,4,5,5-tetramethyl-1,3,2-dioxaborolan-2-yl)phenyl]-3-fluoropyrrolidine-1-sulfonamide (2 g, crude) as a black oil. LCMS: C₁₆H₂₂BClF₂N₂O₄S requires: 422.1, found: m/z=423.2 [M+H]⁺.

### Step-4: Synthesis of tert-butyl 4-{4-[4-(5-chloro-2-fluoro-3-{[(3R)-3-fluoropyrrolidin-1-ylsulfonyl]amino}phenyl)-3-(pyridin-4-yl)pyrazol-1-yl]phenyl}piperazine-1-carboxylate (5)

To a mixture of tert-butyl 4-{4-[4-bromo-3-(pyridin-4-yl)pyrazol-1-yl]phenyl}piperazine-1-carboxylate (2.4 g, 4.955 mmol, 1 equiv) and (3R)—N-[5-chloro-2-fluoro-3-(4,4,5,5-tetramethyl-1,3,2-dioxaborolan-2-yl)phenyl]-3-fluoropyrrolidine-1-sulfonamide (2.09 g, 4.955 mmol, 1 equiv) in dioxane (40 mL) was added Pd(AMPHOS)₂Cl₂ (0.35 g, 0.496 mmol, 0.1 equiv) and CsF (1.51 g, 9.91 mmol, 2 equiv) in H₂O (8 mL). The resulting mixture was stirred at 90° C. for 2 h under a nitrogen atmosphere. The resulting mixture was then concentrated under reduced pressure. The residue was purified by silica gel column chromatography eluted with PE:EtOAc (1:2) to afford tert-butyl 4-{4-[4-(5-chloro-2-fluoro-3-{[(3R)-3-fluoropyrrolidin-1-ylsulfonyl]amino}phenyl)-3-(pyridin-4-yl)pyrazol-1-yl]phenyl}piperazine-1-carboxylate (1.1 g, 26.95%) as a yellow solid. LCMS: C₃₃H₃₆ClF₂N₇O₄S requires: 699.2, found: m/z=700.3 [M+H]⁺.

### Step-5: Synthesis of (3R)—N-(5-chloro-2-fluoro-3-{1-[4-(piperazin-1-yl)phenyl]-3-(pyridin-4-yl)pyrazol-4-yl}phenyl)-3-fluoropyrrolidine-1-sulfonamide Trifluoroacetic Acid Salt (6)

To a mixture of tert-butyl 4-{4-[4-(5-chloro-2-fluoro-3-{[(3R)-3-fluoropyrrolidin-1-ylsulfonyl]amino}phenyl)-3-(pyridin-4-yl)pyrazol-1-yl]phenyl}piperazine-1-carboxylate (1.1 g, 1.571 mmol, 1 equiv) was added TFA (10 mL) at 0° C. The resulting mixture was stirred at 0° C. for one hour. The resulting mixture was then concentrated under reduced pressure. The residue was purified by reverse phase flash chromatography eluted with ACN:H₂O (1:4) to afford (3R)—N-(5-chloro-2-fluoro-3-{1-[4-(piperazin-1-yl)phenyl]-3-(pyridin-4-yl)pyrazol-4-yl}phenyl)-3-fluoropyrrolidine-1-sulfonamide trifluoroacetic acid salt (1.0020 g, 87.71%) as a yellow solid. LCMS: C₂₈H₂₈ClF₂N₇O₂S requires: 599.2, found: m/z=600.2 [M+H]⁺. ¹H NMR (400 MHz, Methanol-d₄) δ 8.74-8.67 (m, 2H), 8.54 (s, 1H), 8.04-7.96 (m, 2H), 7.92-7.84 (m, 2H), 7.72-7.64 (m, 1H), 7.37-7.31 (m, 1H), 7.28-7.19 (m, 2H), 5.28 (m, 1H), 3.66-3.49 (m, 7H), 3.49-3.38 (m, 5H), 2.33-1.98 (m, 2H).

### Synthesis of (3R)—N-[5-chloro-2-fluoro-3-(1-{4-[(3R)-3-methylpiperazin-1-yl]phenyl}-3-(pyridin-4-yl)pyrazol-4-yl)phenyl]-3-fluoropyrrolidine-1-sulfonamide Trifluoroacetic Acid Salt (6)

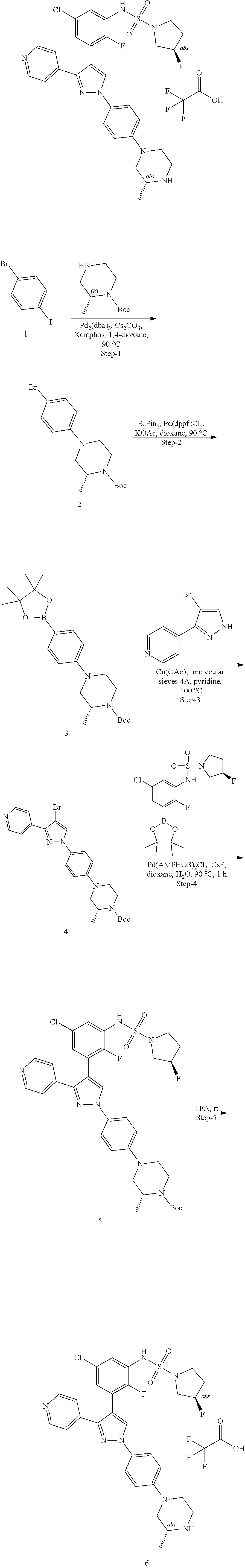

### Step-1: Synthesis of tert-butyl (2R)-4-(4-bromophenyl)-2-methylpiperazine-1-carboxylate (2)

To a mixture of 4-bromoiodobenzene (10 g, 35.347 mmol, 1 equiv) and tert-butyl (2R)-2-methylpiperazine-1-carboxylate (8.5 g, 42.416 mmol, 1.2 equiv) in 1,4-dioxane (150 mL) was added Pd₂(dba)₃ (1.6 g, 1.767 mmol, 0.05 equiv), Xantphos (2.1 g, 3.535 mmol, 0.1 equiv), and Cs₂CO₃ (17.3 g, 53.020 mmol, 1.5 equiv). The resulting mixture was stirred at 90° C. overnight under a nitrogen atmosphere. The resulting mixture was then concentrated under reduced pressure. The residue was purified by reverse phase flash chromatography eluted with PE:EtOAc (5:1) to afford tert-butyl (2R)-4-(4-bromophenyl)-2-methylpiperazine-1-carboxylate (3 g, 23.89%) as a brown solid. LCMS: C₁₆H₂₃BrN₂O₂ requires: 354.0, found: m/z=355.2 [M+H]⁺.

### Step-2: Synthesis of tert-butyl (2R)-2-methyl-4-[4-(4,4,5,5-tetramethyl-1,3,2-dioxaborolan-2-yl)phenyl]piperazine-1-carboxylate (3)

To a mixture of tert-butyl (2R)-4-(4-bromophenyl)-2-methylpiperazine-1-carboxylate (3 g, 8.444 mmol, 1 equiv) and 4,4,5,5-tetramethyl-2-(4,4,5,5-tetramethyl-1,3,2-dioxaborolan-2-yl)-1,3,2-dioxaborolane (B₂Pin₂) (4.3 g, 16.888 mmol, 2 equiv) in 1,4-dioxane (35 mL) was added Pd(dppf)Cl₂ (0.7 g, 0.844 mmol, 0.1 equiv) and KOAc (1.7 g, 16.888 mmol, 2 equiv). The resulting mixture was stirred at 90° C. overnight under a nitrogen atmosphere. The resulting mixture was then filtered. The filtrate was concentrated under reduced pressure. This resulted in tert-butyl (2R)-2-methyl-4-[4-(4,4,5,5-tetramethyl-1,3,2-dioxaborolan-2-yl)phenyl]piperazine-1-carboxylate (3 g, crude) as a brown solid. LCMS: C₂₂H₃₅BN₂04 requires: 402.2, found: m/z=403.2 [M+H]⁺.

### Step-3: Synthesis of tert-butyl (2R)-4-{4-[4-bromo-3-(pyridin-4-yl)pyrazol-1-yl]phenyl}-2-methylpiperazine-1-carboxylate (4)

To a mixture of tert-butyl (2R)-2-methyl-4-[4-(4,4,5,5-tetramethyl-1,3,2-dioxaborolan-2-yl)phenyl]piperazine-1-carboxylate (3 g, 7.456 mmol, 1 equiv) and 4-(4-bromo-1H-pyrazol-3-yl)pyridine (2 g, 8.947 mmol, 1.2 equiv) in pyridine (40 mL) was added Cu(OAc)₂ (2.7 g, 14.912 mmol, 2 equiv) and molecular sieves (4A) (3 g). The resulting mixture was stirred at 100° C. overnight under an oxygen atmosphere. The resulting mixture was then extracted with EtOAc. The combined organic layers were washed with water and dried over anhydrous Na₂SO₄. After filtration, the filtrate was concentrated under reduced pressure. The residue was purified by silica gel column chromatography eluted with PE:EtOAc (3:2) to afford tert-butyl (2R)-4-{4-[4-bromo-3-(pyridin-4-yl)pyrazol-1-yl]phenyl}-2-methylpiperazine-1-carboxylate (900 mg, 21.80%) as an orange oil. LCMS: C₂₄H₂₈BrN₅O₂ requires: 497.1, found: m/z=498.1 [M+H]⁺.

### Step-4: Synthesis of tert-butyl (2R)-4-{4-[4-(5-chloro-2-fluoro-3-{[(3R)-3-fluoropyrrolidin-1-ylsulfonyl]amino}phenyl)-3-(pyridin-4-yl)pyrazol-1-yl]phenyl}-2-methylpiperazine-1-carboxylate (5)

To a mixture of tert-butyl (2R)-4-{4-[4-bromo-3-(pyridin-4-yl)pyrazol-1-yl]phenyl}-2-methylpiperazine-1-carboxylate (900 mg, 1.806 mmol, 1 equiv) and (3R)—N-[5-chloro-2-fluoro-3-(4,4,5,5-tetramethyl-1,3,2-dioxaborolan-2-yl)phenyl]-3-fluoropyrrolidine-1-sulfonamide (763 mg, 1.806 mmol, 1 equiv) in dioxane (15 mL) was added Pd(AMPHOS)₂Cl₂ (128 mg, 0.181 mmol, 0.1 equiv) and CsF (549 mg, 3.612 mmol, 2 equiv) in H₂O (3 mL). The resulting mixture was stirred at 90° C. for one hour under a nitrogen atmosphere. The resulting mixture was then concentrated under reduced pressure. The residue was purified by silica gel column chromatography eluted with CH₂Cl₂:EtOAc (5:1) to afford tert-butyl (2R)-4-{4-[4-(5-chloro-2-fluoro-3-f{[(3R)-3-fluoropyrrolidin-1-ylsulfonyl]amino}phenyl)-3-(pyridin-4-yl)pyrazol-1-yl]phenyl}-2-methylpiperazine-1-carboxylate (460 mg, 19.62%) as a brown solid. LCMS: C₃₄H₃₈ClF₂N₇O₄S requires: 713.2, found: m/z=714.2 [M+H]⁺.

### Step-5: Synthesis of (3R)—N-[5-chloro-2-fluoro-3-(1-{4-[(3R)-3-methylpiperazin-1-yl]phenyl}-3-(pyridin-4-yl)pyrazol-4-yl)phenyl]-3-fluoropyrrolidine-1-sulfonamide Trifluoroacetic Acid Salt (6)

A mixture of tert-butyl (2R)-4-{4-[4-(5-chloro-2-fluoro-3-{[(3R)-3-fluoropyrrolidin-1-ylsulfonyl]amino}phenyl)-3-(pyridin-4-yl)pyrazol-1-yl]phenyl}-2-methylpiperazine-1-carboxylate (600 mg, 0.840 mmol, 1 equiv) in TFA (6 mL) was stirred at room temperature for one hour. The resulting mixture was then concentrated under reduced pressure. The crude product was purified by prep-HPLC with the following conditions: Column: Xcelect CSH F-pheny OBD Column 19×250 mm; Mobile Phase A: Water (0.05% TFA), Mobile Phase B: ACN; Flow rate: 20 mL/min; Gradient: 35% B to 50% B in 15 min; Wave Length: 254/220 nm to afford (3R)—N-[5-chloro-2-fluoro-3-(1-{4-[(3R)-3-methylpiperazin-1-yl]phenyl}-3-(pyridin-4-yl)pyrazol-4-yl)phenyl]-3-fluoropyrrolidine-1-sulfonamide trifluoroacetic acid salt (136.2 mg, 20.04%) as a yellow solid. LCMS: C₂₉H₃₀ClF₂N₇O₂S requires: 613.1, found: m/z=614.2 [M+H]⁺. ¹H NMR (400 MHz, Methanol-d₄) δ 8.73-8.67 (m, 2H), 8.57-8.52 (m, 1H), 8.05-7.99 (m, 2H), 7.93-7.85 (m, 2H), 7.71-7.64 (m, 1H), 7.37-7.31 (m, 1H), 7.27-7.20 (m, 2H), 5.37-5.18 (m, 1H), 3.99-3.86 (m, 2H), 3.68-3.34 (m, 4H), 3.38-3.33 (m, 3H), 3.14-3.06 (m, 1H), 2.95-2.84 (m, 1H), 2.30-2.03 (m, 2H), 1.48-1.42 (m, 3H).

### Synthesis of (3R)—N-[5-chloro-2-fluoro-3-(1-{5-[(3R)-3-methylpiperazin-1-yl]pyridin-2-yl}-3-(pyridin-4-yl)pyrazol-4-yl)phenyl]-3-fluoropyrrolidine-1-sulfonamide Trifluoroacetic Acid Salt (5)

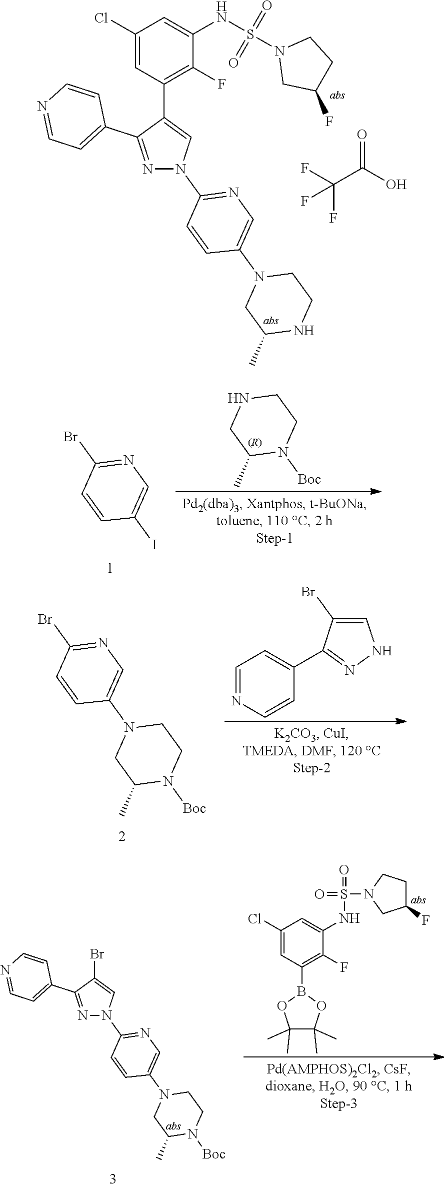

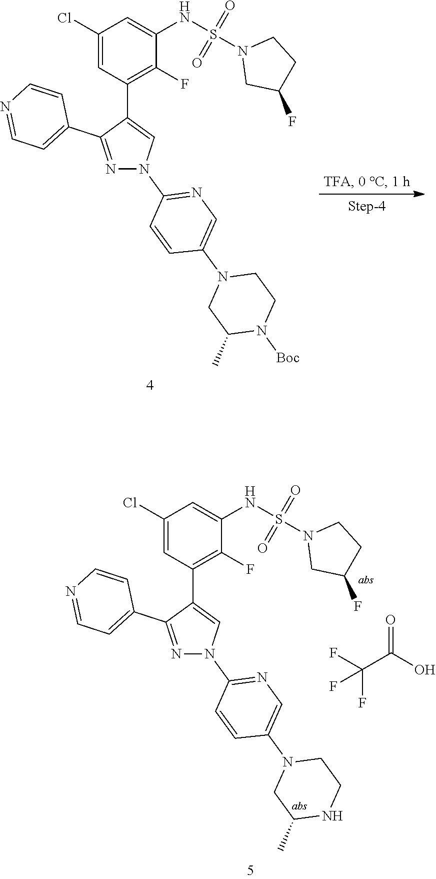

### Step-1: Synthesis of tert-butyl (2R)-4-(6-bromopyridin-3-yl)-2-methylpiperazine-1-carboxylate (2)

To a mixture of 2-bromo-5-iodopyridine (18.3 g, 64.410 mmol, 1.29 equiv) and tert-butyl (2R)-2-methylpiperazine-1-carboxylate (10 g, 49.930 mmol, 1 equiv) in toluene (200 mL) was added t-BuONa (14.4 g, 149.790 mmol, 3 equiv), Xantphos (3.47 g, 5.992 mmol, 0.12 equiv), and Pd₂(dba)₃ (2.3 g, 2.497 mmol, 0.05 equiv). The resulting mixture was stirred at 110° C. for 2 h under a nitrogen atmosphere. The resulting mixture was then concentrated under reduced pressure. The residue was purified by silica gel column chromatography eluted with PE:EtOAc (4:1) to afford tert-butyl (2R)-4-(6-bromopyridin-3-yl)-2-methylpiperazine-1-carboxylate (11 g, 55.6%) as a yellow solid. LCMS: C₁₅H₂₂BrN₃O₂ requires: 355.0, found: m/z=356.1 [M+H]⁺.

### Step-2: Synthesis of tert-butyl (2R)-4-{6-[4-bromo-3-(pyridin-4-yl)pyrazol-1-yl]pyridin-3-yl}-2-methylpiperazine-1-carboxylate (3)

To a mixture of tert-butyl (2R)-4-(6-bromopyridin-3-yl)-2-methylpiperazine-1-carboxylate (6 g, 16.888 mmol, 1 equiv) and 4-(4-bromo-1H-pyrazol-3-yl)pyridine (5.7 g, 25.332 mmol, 1.5 equiv) in DMF (80 mL) was added K₂CO₃ (7.0 g, 50.664 mmol, 3 equiv), TMEDA (0.79 g, 6.755 mmol, 0.4 equiv), and CuI (640 mg, 3.378 mmol, 0.2 equiv). The resulting mixture was stirred at 120° C. overnight under a nitrogen atmosphere. The resulting mixture was then extracted with EtOAc. The combined organic layers were washed with water and dried over anhydrous Na₂SO₄. After filtration, the filtrate was concentrated under reduced pressure. The residue was purified by silica gel column chromatography eluted with PE:EtOAc (1:3) to afford tert-butyl (2R)-4-{6-[4-bromo-3-(pyridin-4-yl)pyrazol-1-yl]pyridin-3-yl}-2-methylpiperazine-1-carboxylate (5 g, 53.35%) as a brown solid. LCMS: C₂₃H₂₇BrN₆O₂ requires: 498.1, found: m/z=499.2 [M+H]⁺.

### Step-3: Synthesis of tert-butyl (2R)-4-{6-[4-(5-chloro-2-fluoro-3-{[(3R)-3-fluoropyrrolidin-1-ylsulfonyl]amino}phenyl)-3-(pyridin-4-yl)pyrazol-1-yl]pyridin-3-yl}-2-methylpiperazine-1-carboxylate (4)

To a mixture of tert-butyl (2R)-4-{6-[4-bromo-3-(pyridin-4-yl)pyrazol-1-yl]pyridin-3-yl}-2-methylpiperazine-1-carboxylate (1.2 g, 2.403 mmol, 1 equiv) and (3R)—N-[5-chloro-2-fluoro-3-(4,4,5,5-tetramethyl-1,3,2-dioxaborolan-2-yl)phenyl]-3-fluoropyrrolidine-1-sulfonamide (3.1 g, 7.209 mmol, 3 equiv) in dioxane (15 mL) was added Pd(AMPHOS)₂Cl₂ (170 mg, 0.240 mmol, 0.1 equiv) and CsF (730 mg, 4.806 mmol, 2 equiv) in H₂O (3 mL). The resulting mixture was stirred at 90° C. for 1 h under a nitrogen atmosphere. The resulting mixture was then concentrated under reduced pressure. The residue was purified by silica gel column chromatography eluted with PE:EtOAc (3:1) to afford tert-butyl (2R)-4-{6-[4-(5-chloro-2-fluoro-3-{[(3R)-3-fluoropyrrolidin-1-ylsulfonyl]amino}phenyl)-3-(pyridin-4-yl)pyrazol-1-yl]pyridin-3-yl}-2-methylpiperazine-1-carboxylate (690 mg, 35.33%) as a yellow solid. LCMS: C₃₃H₃₇ClF₂N₈O₄S requires: 714.2, found: m/z=715.2 [M+H]⁺.

### Step-4: Synthesis of (3R)—N-[5-chloro-2-fluoro-3-(1-{5-[(3R)-3-methylpiperazin-1-yl]pyridin-2-yl}-3-(pyridin-4-yl)pyrazol-4-yl)phenyl]-3-fluoropyrrolidine-1-sulfonamide Trifluoroacetic Acid Salt (5)

A mixture of tert-butyl (2R)-4-{6-[4-(5-chloro-2-fluoro-3-{[(3R)-3-fluoropyrrolidin-1-ylsulfonyl]amino}phenyl)-3-(pyridin-4-yl)pyrazol-1-yl]pyridin-3-yl}-2-methylpiperazine-1-carboxylate (680 mg, 0.951 mmol, 1 equiv) in TFA (7 mL) was stirred at 0° C. for one hour. The resulting mixture was then concentrated under reduced pressure. The residue was purified by C18 reverse phase flash chromatography eluted with ACN:H₂O (4:1) to afford (3R)—N-[5-chloro-2-fluoro-3-(1-{5-[(3R)-3-methylpiperazin-1-yl]pyridin-2-yl}-3-(pyridin-4-yl)pyrazol-4-yl)phenyl]-3-fluoropyrrolidine-1-sulfonamide trifluoroacetic acid salt (427.6 mg, 59.09%) as a yellow solid. LCMS: C₂₈H₂₉ClF₂N₈O₂S requires: 614.1, found: m/z=615.1 [M+H]⁺. ¹H NMR (300 MHz, Methanol-d₄) δ 8.86-8.80 (m, 1H), 8.79-8.71 (m, 2H), 8.33-8.26 (m, 1H), 8.16-8.04 (m, 3H), 7.79-7.65 (m, 2H), 7.37-7.29 (m, 1H), 5.40-5.34 (m, 1H), 4.05-3.90 (m, 2H), 3.65-3.35 (m, 7H), 3.26-3.11 (m, 1H), 3.03-2.89 (m, 1H), 2.33-1.98 (m, 2H), 1.50-1.41 (m, 3H).

### Synthesis of (3R)—N-(5-chloro-2-fluoro-3-{1-[5-(piperazin-1-yl)pyridin-2-yl]-3-(pyridin-4-yl)pyrazol-4-yl}phenyl)-3-fluoropyrrolidine-1-sulfonamide Trifluoroacetic Acid Salt (3)

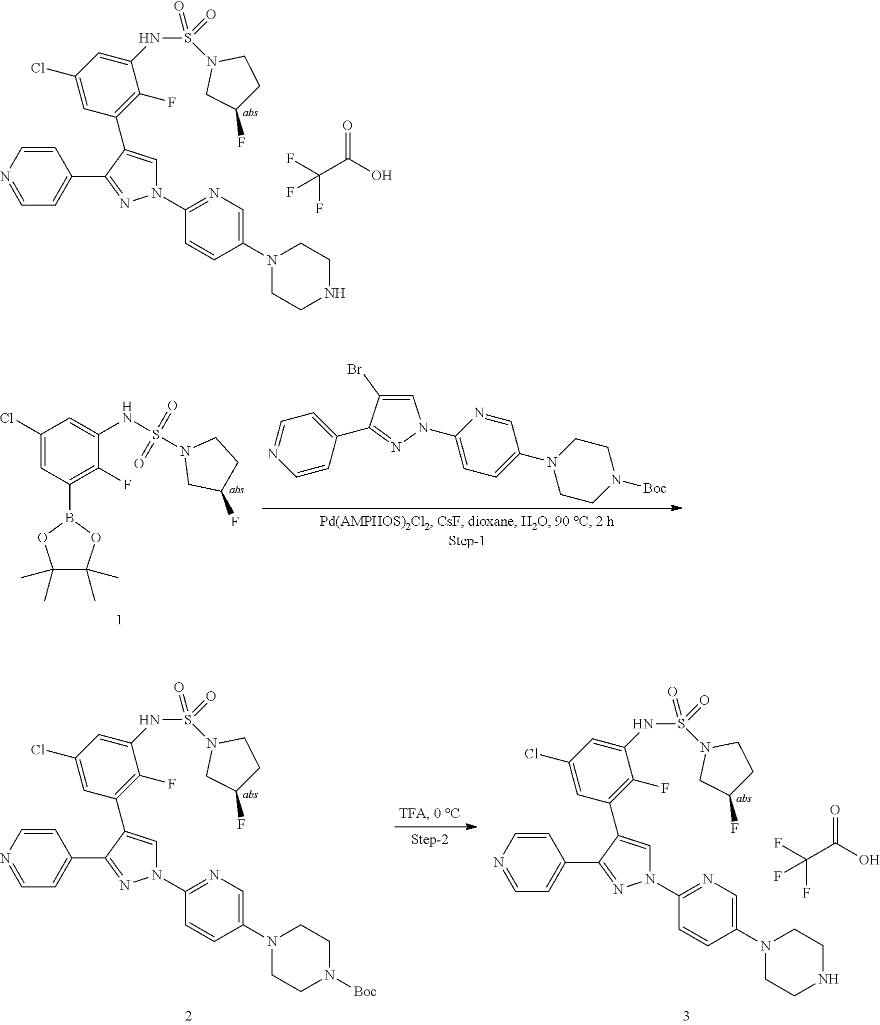

### Step-1: Synthesis of tert-butyl 4-{6-[4-(5-chloro-2-fluoro-3-{[(3R)-3-fluoropyrrolidin-1-ylsulfonyl]amino}phenyl)-3-(pyridin-4-yl)pyrazol-1-yl]pyridin-3-yl}piperazine-1-carboxylate (2)

To a mixture of (3R)—N-[5-chloro-2-fluoro-3-(4,4,5,5-tetramethyl-1,3,2-dioxaborolan-2-yl)phenyl]-3-fluoropyrrolidine-1-sulfonamide (1.9 g, 4.495 mmol, 1 equiv) and tert-butyl 4-{6-[4-bromo-3-(pyridin-4-yl)pyrazol-1-yl]pyridin-3-yl}piperazine-1-carboxylate (2.2 g, 4.495 mmol, 1 equiv) in dioxane (30 mL) was added CsF (1.37 g, 8.99 mmol, 2 equiv) in H₂O (6 mL) and Pd(AMPHOS)₂Cl₂ (0.32 g, 0.449 mmol, 0.1 equiv). The resulting mixture was stirred at 90° C. for 2 h under a nitrogen atmosphere. The resulting mixture was then concentrated under vacuum. The residue was purified by silica gel column chromatography eluted with PE:EtOAc (1:3) to afford tert-butyl 4-{6-[4-(5-chloro-2-fluoro-3-{[(3R)-3-fluoropyrrolidin-1-ylsulfonyl]amino}phenyl)-3-(pyridin-4-yl)pyrazol-1-yl]pyridin-3-yl}piperazine-1-carboxylate (1.5 g, 45.21%) as an off-white solid. LCMS: C₃₂H₃₅ClF₂N₈O₄S requires: 700.2, found: m/z=701.2 [M+H]⁺.

### Step-2: Synthesis of (3R)—N-(5-chloro-2-fluoro-3-{1-[5-(piperazin-1-yl)pyridin-2-yl]-3-(pyridin-4-yl)pyrazol-4-yl}phenyl)-3-fluoropyrrolidine-1-sulfonamide Trifluoroacetic Acid Salt (3)

A mixture of tert-butyl 4-{6-[4-(5-chloro-2-fluoro-3-{[(3R)-3-fluoropyrrolidin-1-ylsulfonyl]amino}phenyl)-3-(pyridin-4-yl)pyrazol-1-yl]pyridin-3-yl}piperazine-1-carboxylate (1 g, 1.426 mmol, 1 equiv) in TFA (10 mL) was stirred at 0° C. for 2 h. The resulting mixture was then concentrated under vacuum. The residue was purified by reverse phase flash chromatography eluted with ACN:H₂O (1:4) to afford (3R)—N-(5-chloro-2-fluoro-3-{1-[5-(piperazin-1-yl)pyridin-2-yl]-3-(pyridin-4-yl)pyrazol-4-yl}phenyl)-3-fluoropyrrolidine-1-sulfonamide trifluoroacetic acid salt (468.7 mg, 53.04%) as a yellow solid. LCMS: C₂₇H₂₇ClF₂N₈O₂S requires: 600.2, found: m/z=601.1 [M+H]⁺. ¹H NMR (400 MHz, Methanol-d₄) δ 8.81 (s, 1H), 8.76-8.68 (m, 2H), 8.31-8.25 (m, 1H), 8.13-8.06 (m, 3H), 7.76-7.63 (m, 2H), 7.35-7.27 (m, 1H), 5.39-5.17 (m, 1H), 3.65-3.48 (m, 7H), 3.47-3.37 (m, 5H), 2.31-1.94 (m, 2H).

### Synthesis of N-[5-chloro-2-fluoro-3-(1-{4-[(3R)-3-methylpiperazin-1-yl]phenyl}-3-(pyridin-4-yl)pyrazol-4-yl)phenyl]pyrrolidine-1-sulfonamide Trifluoroacetic Acid Salt (3)

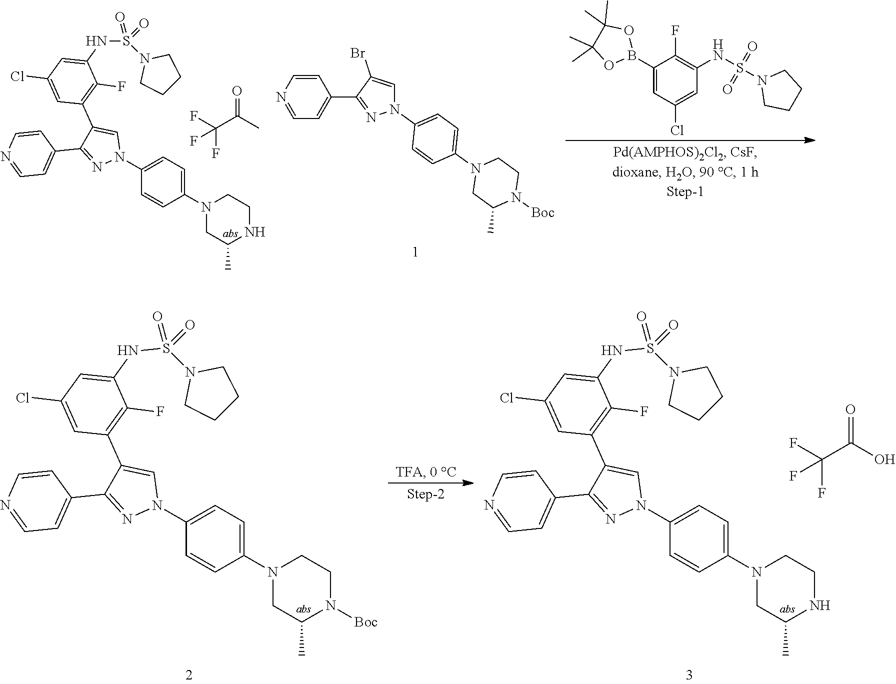

### Step-1: Synthesis of tert-butyl (2R)-4-[4-(4-{5-chloro-2-fluoro-3-[(pyrrolidine-1-sulfonyl)amino]phenyl}-3-(pyridin-4-yl)pyrazol-1-yl)phenyl]-2-methylpiperazine-1-carboxylate (2)

To a mixture of tert-butyl (2R)-4-{4-[4-bromo-3-(pyridin-4-yl)pyrazol-1-yl]phenyl}-2-methylpiperazine-1-carboxylate (500 mg, 1.003 mmol, 1 equiv) and N-[5-chloro-2-fluoro-3-(4,4,5,5-tetramethyl-1,3,2-dioxaborolan-2-yl)phenyl]pyrrolidine-1-sulfonamide (406 mg, 1.003 mmol, 1 equiv) in dioxane (15 mL) was added Pd(AMPHOS)₂Cl₂ (71 mg, 0.1 mmol, 0.1 equiv) and CsF (305 mg, 2.006 mmol, 2 equiv) in H₂O (3 mL). The resulting mixture was stirred at 90° C. for one hour under a nitrogen atmosphere. The resulting mixture was then concentrated under reduced pressure. The residue was purified by silica gel column chromatography eluted with CH₂Cl₂:EtOAc (2:1) to afford tert-butyl (2R)-4-[4-(4-{5-chloro-2-fluoro-3-[(pyrrolidine-1-sulfonyl)amino]phenyl}-3-(pyridin-4-yl)pyrazol-1-yl)phenyl]-2-methylpiperazine-1-carboxylate (300 mg, 30.07%) as a brown oil. LCMS: C₃₄H₃₉ClFN₇O₄S requires: 695.2, found: m/z=696.2 [M+H]⁺.

### Step-2: Synthesis of N-[5-chloro-2-fluoro-3-(1-{4-[(3R)-3-methylpiperazin-1-yl]phenyl}-3-(pyridin-4-yl)pyrazol-4-yl)phenyl]pyrrolidine-1-sulfonamide Trifluoroacetic Acid Salt (3)

A mixture of tert-butyl (2R)-4-[4-(4-{5-chloro-2-fluoro-3-[(pyrrolidine-1-sulfonyl)amino]phenyl}-3-(pyridin-4-yl)pyrazol-1-yl)phenyl]-2-methylpiperazine-1-carboxylate (280 mg, 0.402 mmol, 1 equiv) in TFA (3 mL) was stirred at 0° C. for one hour. The resulting mixture was then concentrated under reduced pressure. The crude product was purified by prep-HPLC with the following conditions: Column: Xselect CSH C18 OBD Column 30×150 mm 5 m; Mobile Phase A: Water (0.05% TFA), Mobile Phase B: ACN; Flow rate: 60 mL/min; Gradient: 5% B to 5% B in 2 min, 10% B to 25% B in 15 min; Wave Length: 254/220 nm to afford N-[5-chloro-2-fluoro-3-(1-{4-[(3R)-3-methylpiperazin-1-yl]phenyl}-3-(pyridin-4-yl)pyrazol-4-yl)phenyl]pyrrolidine-1-sulfonamide trifluoroacetic acid salt (188.8 mg, 22.73%) as a yellow solid. LCMS: C₂₉H₃₁ClFN₇O₂S requires: 595.1, found: m/z=596.2 [M+H]⁺. ¹H NMR (300 MHz, Methanol-d₄) δ 8.80-8.72 (m, 2H), 8.63-8.57 (m, 1H), 8.19-8.11 (m, 2H), 7.94-7.85 (m, 2H), 7.69-7.59 (m, 1H), 7.41-7.32 (m, 1H), 7.29-7.19 (m, 2H), 4.00-3.86 (m, 2H), 3.61-3.51 (m, 2H), 3.50-3.43 (m, 2H), 3.43-3.28 (m, 3H), 3.21-3.02 (m, 1H), 2.97-2.84 (m, 1H), 1.99-1.84 (m, 4H), 1.57-1.41 (m, 3H).

### Synthesis of N-[5-chloro-2-fluoro-3-(1-{5-[(3R)-3-methylpiperazin-1-yl]pyridin-2-yl}-3-(pyridin-4-yl)pyrazol-4-yl)phenyl]pyrrolidine-1-sulfonamide Trifluoroacetic Acid Salt (3)

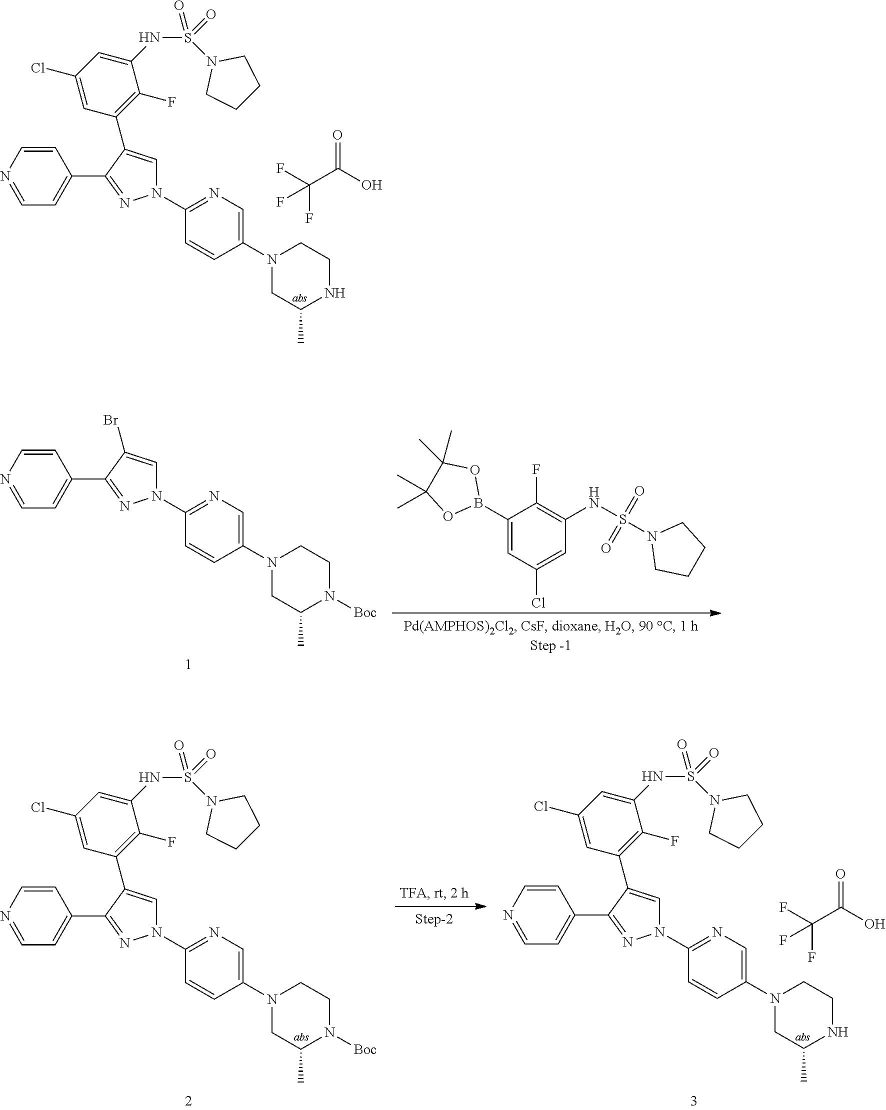

### Step-1: Synthesis of tert-butyl (2R)-4-[6-(4-{5-chloro-2-fluoro-3-[(pyrrolidine-1-sulfonyl)amino]phenyl}-3-(pyridin-4-yl)pyrazol-1-yl)pyridin-3-yl]-2-methylpiperazine-1-carboxylate (2)

To a mixture of tert-butyl (2R)-4-{6-[4-bromo-3-(pyridin-4-yl)pyrazol-1-yl]pyridin-3-yl}-2-methylpiperazine-1-carboxylate (700 mg, 1.402 mmol, 1 equiv) and N-[5-chloro-2-fluoro-3-(4,4,5,5-tetramethyl-1,3,2-dioxaborolan-2-yl)phenyl]pyrrolidine-1-sulfonamide (567 mg, 1.402 mmol, 1 equiv) in dioxane (20 mL) was added Pd(AMPHOS)₂Cl₂ (99 mg, 0.140 mmol, 0.1 equiv) and CsF (430 mg, 2.804 mmol, 2 equiv) in H₂O (4 mL). The resulting mixture was stirred at 90° C. for one hour under a nitrogen atmosphere. The resulting mixture was then concentrated under reduced pressure. The residue was purified by silica gel column chromatography eluted with CH₂Cl₂:EtOAc (2:1) to afford tert-butyl (2R)-4-[6-(4-{5-chloro-2-fluoro-3-[(pyrrolidine-1-sulfonyl)amino]phenyl}-3-(pyridin-4-yl)pyrazol-1-yl)pyridin-3-yl]-2-methylpiperazine-1-carboxylate (400 mg, 34.79%) as a brown solid. LCMS: C₃₃H₃₈ClFN₈O₄S requires: 696.2, found: m/z=697.2 [M+H]⁺.

### Step-2: Synthesis of N-[5-chloro-2-fluoro-3-(1-{5-[(3R)-3-methylpiperazin-1-yl]pyridin-2-yl}-3-(pyridin-4-yl)pyrazol-4-yl)phenyl]pyrrolidine-1-sulfonamide Trifluoroacetic Acid Salt (3)

A mixture of tert-butyl (2R)-4-[6-(4-{5-chloro-2-fluoro-3-[(pyrrolidine-1-sulfonyl)amino]phenyl}-3-(pyridin-4-yl)pyrazol-1-yl)pyridin-3-yl]-2-methylpiperazine-1-carboxylate (390 mg, 0.559 mmol, 1 equiv) in TFA (4 mL) was stirred at room temperature for 2 h. The resulting mixture was then concentrated under reduced pressure. The crude product was purified by prep-HPLC with the following conditions: Column: XBridge Prep Phenyl OBD Column 19×250 mm; Mobile Phase A: Water (0.05% TFA), Mobile Phase B: ACN; Flow rate: 25 mL/min; Gradient: 23% B to 33% B in 10 min; Wave Length: 254/297 nm to afford N-[5-chloro-2-fluoro-3-(1-{5-[(3R)-3-methylpiperazin-1-yl]pyridin-2-yl}-3-(pyridin-4-yl)pyrazol-4-yl)phenyl]pyrrolidine-1-sulfonamide trifluoroacetic acid salt (246.8 mg, 60.49%) as a yellow solid. LCMS: C₂₈H₃₀ClFN₈O₂S requires: 596.1, found: m/z=597.2 [M+H]⁺. ¹H NMR (300 MHz, Methanol-d₄) δ 8.88-8.82 (m, 1H), 8.81-8.73 (m, 2H), 8.33-8.27 (m, 1H), 8.16-8.06 (m, 3H), 7.80-7.70 (m, 1H), 7.69-7.60 (m, 1H), 7.38-7.29 (m, 1H), 4.05-3.90 (m, 2H), 3.59-3.53 (m, 2H), 3.46-3.35 (m, 3H), 3.34-3.28 (m, 2H), 3.26-3.09 (m, 1H), 3.03-2.89 (m, 1H), 1.99-1.86 (m, 4H), 1.56-1.42 (m, 3H).

### Synthesis of N-(5-chloro-2-fluoro-3-{1-[4-(piperazin-1-yl)phenyl]-3-(pyridin-4-yl)pyrazol-4-yl}phenyl)cyclopentanesulfonamide Trifluoroacetic Acid Salt (5)

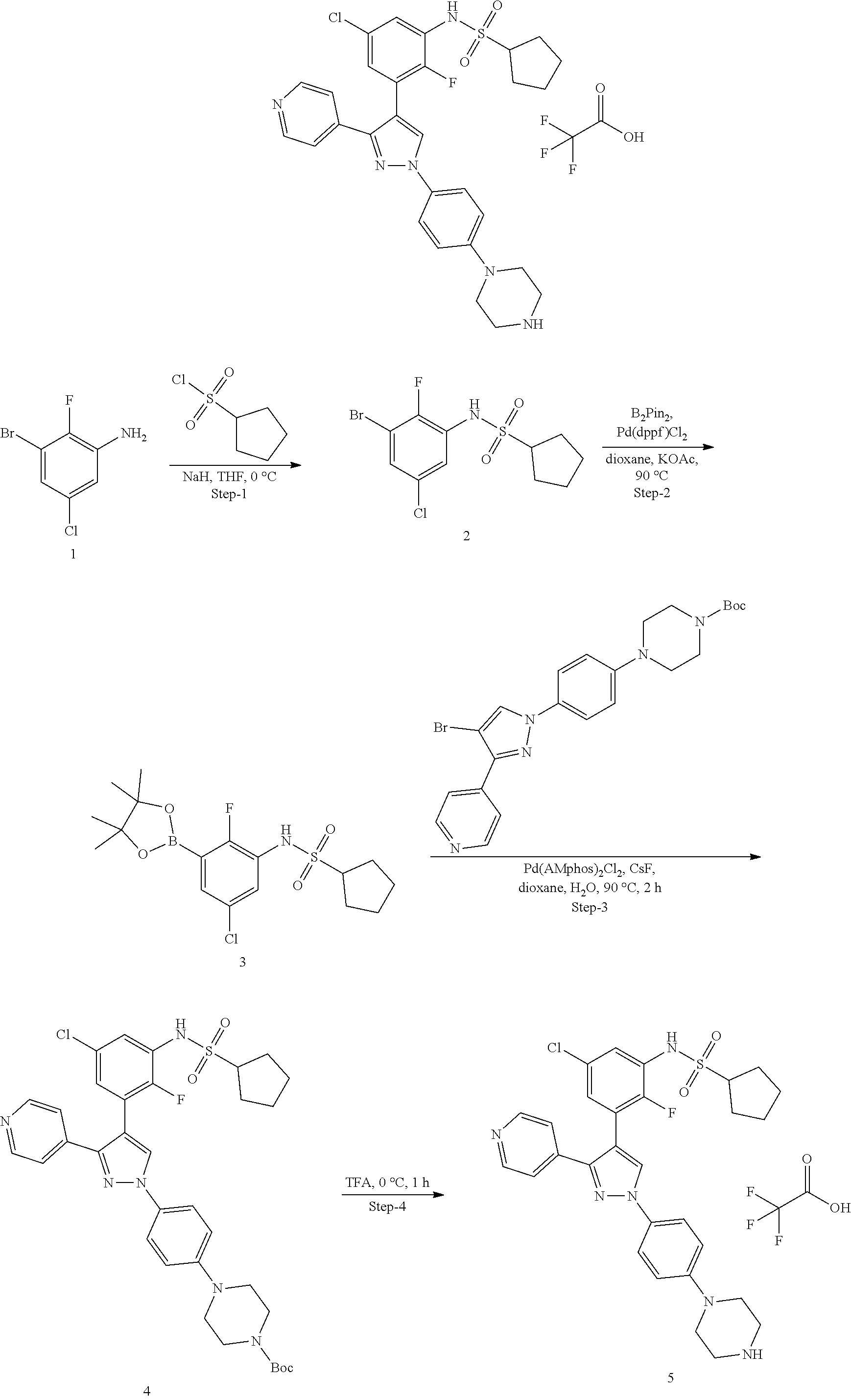

### Step-1: Synthesis of N-(3-bromo-5-chloro-2-fluorophenyl)cyclopentanesulfonamide (2)

To a mixture of NaH (1 g, 26.730 mmol, 2 equiv, 60%) in THF (50 mL) was added 3-bromo-5-chloro-2-fluoroaniline (3 g, 13.365 mmol, 1 equiv). The resulting mixture was then stirred for one hour at 0° C. under a nitrogen atmosphere. To the above mixture was then added cyclopentanesulfonyl chloride (4.5 g, 26.730 mmol, 2 equiv) at 0° C. The resulting mixture was then stirred at room temperature overnight under a nitrogen atmosphere. The reaction was then quenched by the addition of water at 0° C. The resulting mixture was concentrated under reduced pressure. The residue was purified by silica gel column chromatography eluted with PE:DCM (1:3) to afford N-(3-bromo-5-chloro-2-fluorophenyl)cyclopentanesulfonamide (630 mg, 11.90%) as an off-white solid. LCMS: C₁₁H₁₂BrClFNO₂S requires: 354.9, found: m/z=354.1 [M−H]⁻.

### Step-2: Synthesis of N-[5-chloro-2-fluoro-3-(4,4,5,5-tetramethyl-1,3,2-dioxaborolan-2-yl)phenyl]cyclopentanesulfonamide (3)

To a mixture of N-(3-bromo-5-chloro-2-fluorophenyl)cyclopentanesulfonamide (630 mg, 1.767 mmol, 1 equiv) and 4,4,5,5-tetramethyl-2-(4,4,5,5-tetramethyl-1,3,2-dioxaborolan-2-yl)-1,3,2-dioxaborolane (B₂Pin₂) (897 mg, 3.534 mmol, 2 equiv) in dioxane (10 mL) was added KOAc (347 mg, 3.534 mmol, 2 equiv) and Pd(dppf)Cl₂ (129.26 mg, 0.177 mmol, 0.1 equiv). The resulting mixture was stirred at 90° C. overnight under a nitrogen atmosphere. The resulting mixture was then filtered. The filtrate was concentrated under reduced pressure. This resulted in N-[5-chloro-2-fluoro-3-(4,4,5,5-tetramethyl-1,3,2-dioxaborolan-2-yl)phenyl]cyclopentanesulfonamide (600 mg, crude) as a black oil. LCMS: C₁₇H₂₄BClFNO₄S requires: 403.1, found: m/z=404.2 [M+H]⁺.

### Step-3: Synthesis of tert-butyl 4-(4-(4-(5-chloro-3-(cyclopentanesulfonamido)-2-fluorophenyl)-3-(pyridin-4-yl)-1H-pyrazol-1-yl)phenyl)piperazine-1-carboxylate (4)

To a mixture of N-[5-chloro-2-fluoro-3-(4,4,5,5-tetramethyl-1,3,2-dioxaborolan-2-yl)phenyl]cyclopentanesulfonamide (600 mg, 1.486 mmol, 1 equiv) and tert-butyl 4-{4-[4-bromo-3-(pyridin-4-yl)pyrazol-1-yl]phenyl}piperazine-1-carboxylate (720 mg, 1.486 mmol, 1 equiv) in dioxane (10 mL) was added Pd(AMPHOS)₂Cl₂ (105 mg, 0.149 mmol, 0.1 equiv) and CsF (452 mg, 2.972 mmol, 2 equiv) in H₂O (2 mL). The resulting mixture was stirred at 90° C. for 2 h under a nitrogen atmosphere. The resulting mixture was then concentrated under reduced pressure. The residue was purified by silica gel column chromatography eluted with CH₂Cl₂:EtOAc (1:1) to afford tert-butyl 4-{4-[4-(5-chloro-3-cyclopentanesulfonamido-2-fluorophenyl)-3-(pyridin-4-yl)pyrazol-1-yl]phenyl}piperazine-1-carboxylate (200 mg, 17.78%) as an off-white solid. LCMS: C₃₄H₃₈ClFN₆O₄S requires: 680.2, found: m/z=681.1 [M+H]⁺.

### Step-4: Synthesis of N-(5-chloro-2-fluoro-3-{1-[4-(piperazin-1-yl)phenyl]-3-(pyridin-4-yl)pyrazol-4-yl}phenyl)cyclopentanesulfonamide Trifluoroacetic Acid Salt (5)

A mixture of tert-butyl 4-{4-[4-(5-chloro-3-cyclopentanesulfonamido-2-fluorophenyl)-3-(pyridin-4-yl)pyrazol-1-yl]phenyl}piperazine-1-carboxylate (200 mg, 0.294 mmol, 1 equiv) in TFA (2 mL) was stirred at 0° C. for one hour. The resulting mixture was then concentrated under reduced pressure and lyophilized. This resulted in N-(5-chloro-2-fluoro-3-{1-[4-(piperazin-1-yl)phenyl]-3-(pyridin-4-yl)pyrazol-4-yl}phenyl)cyclopentanesulfonamide trifluoroacetic acid salt (126.1 mg, crude) as a yellow solid. LCMS: C₂₉H₃₀ClFN₆O₂S requires: 580.1, found: m/z=581.2 [M+H]⁺. ¹H NMR (400 MHz, Methanol-d₄) δ 8.78-8.72 (m, 2H), 8.62-8.58 (m, 1H), 8.17-8.11 (m, 2H), 7.94-7.86 (m, 2H), 7.71-7.63 (m, 1H), 7.46-7.39 (m, 1H), 7.28-7.20 (m, 2H), 3.72-3.59 (m, 1H), 3.58-3.50 (m, 4H), 3.47-3.40 (m, 4H), 2.09-1.91 (m, 4H), 1.83-1.78 (m, 2H), 1.69-1.65 (m, 2H).

### Synthesis of rac-(2R)—N-(5-chloro-2-fluoro-3-{1-[4-(piperazin-1-yl)phenyl]-3-(pyridin-4-yl)pyrazol-4-yl}phenyl)butane-2-sulfonamide Trifluoroacetic Acid Salt (5)

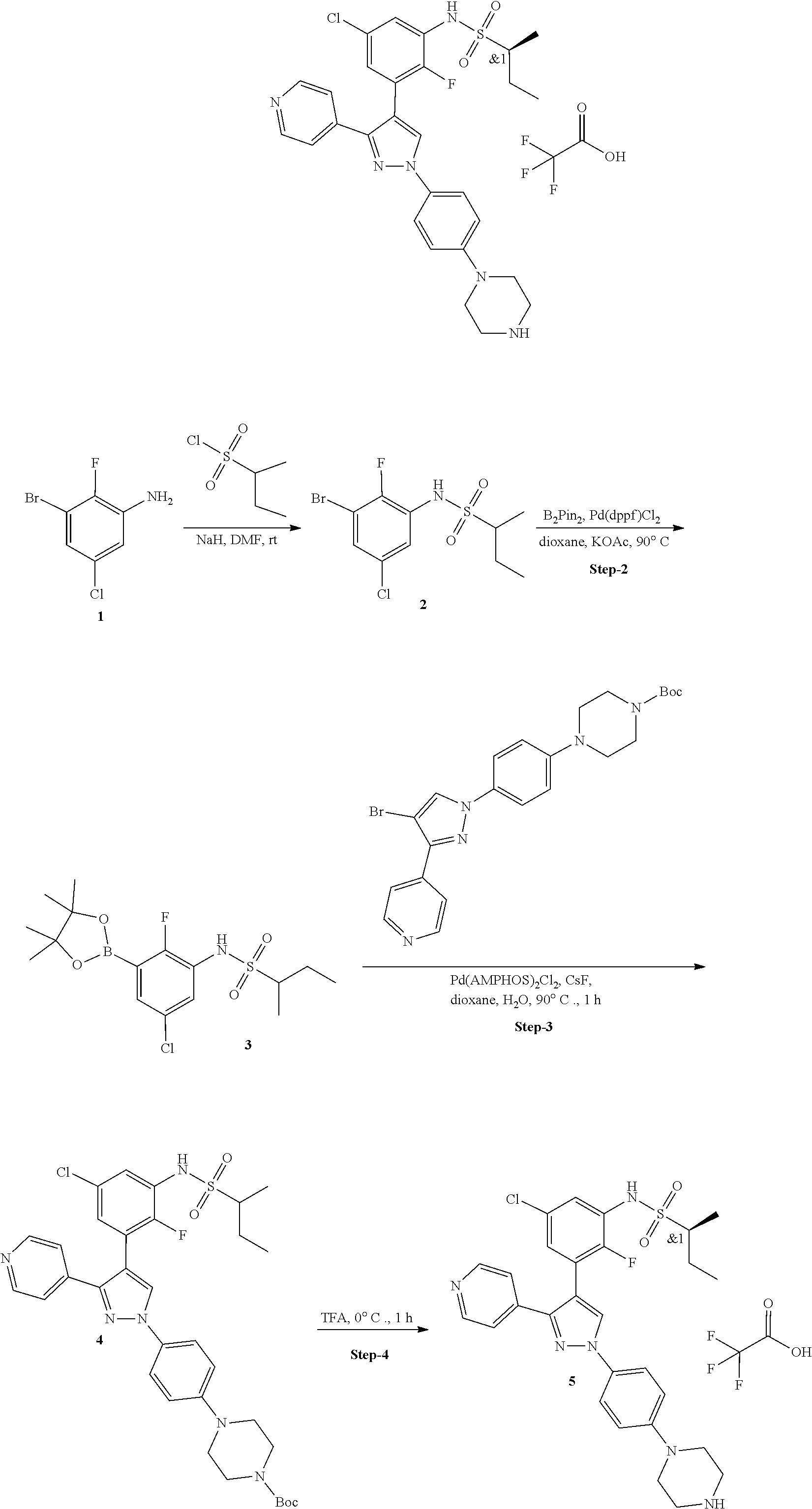

### Step-1: Synthesis of N-(3-bromo-5-chloro-2-fluorophenyl)butane-2-sulfonamide (2)

To a mixture of NaH (891 mg, 22.276 mmol, 2 equiv, 60%) in DMF (50 mL) was added 3-bromo-5-chloro-2-fluoroaniline (2.5 g, 11.138 mmol, 1 equiv). The resulting mixture was stirred at 0° C. for one hour under a nitrogen atmosphere. To the above mixture was added butane-2-sulfonyl chloride (3.5 g, 22.276 mmol, 2 equiv) at 0° C. The resulting mixture was stirred at room temperature overnight under a nitrogen atmosphere. The reaction was then quenched with water at 0° C. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with PE:CH₂Cl₂ (1:1) to afford N-(3-bromo-5-chloro-2-fluorophenyl)butane-2-sulfonamide (650 mg, 15.24%) as a brown semi-solid. LCMS: C₁₀H₁₂BrClFNO₂S Srequires: 342.9, found: m/z=342.0 [M−H]⁻.

### Step-2: Synthesis of N-[5-chloro-2-fluoro-3-(4,4,5,5-tetramethyl-1,3,2-dioxaborolan-2-yl)phenyl]butane-2-sulfonamide (3)

To a mixture of N-(3-bromo-5-chloro-2-fluorophenyl)butane-2-sulfonamide (650 mg, 1.886 mmol, 1 equiv) and 4,4,5,5-tetramethyl-2-(4,4,5,5-tetramethyl-1,3,2-dioxaborolan-2-yl)-1,3,2-dioxaborolane (B₂Pin₂) (958 mg, 3.772 mmol, 2 equiv) in dioxane (10 mL) was added Pd(dppf)Cl₂ (138 mg, 0.189 mmol, 0.1 equiv) and KOAc (370 mg, 3.772 mmol, 2 equiv). The resulting mixture was stirred at 90° C. overnight under a nitrogen atmosphere. The resulting mixture was then filtered. The filtrate was concentrated under reduced pressure. This resulted in N-[5-chloro-2-fluoro-3-(4,4,5,5-tetramethyl-1,3,2-dioxaborolan-2-yl)phenyl]butane-2-sulfonamide (650 mg, crude) as a black oil. LCMS: C₁₆H₂₄BClFNO₄S requires: 391.1, found: m/z=392.2 [M+H]⁺.

### Step-3: Synthesis of tert-butyl 4-(4-{4-[3-(butane-2-sulfonamido)-5-chloro-2-fluorophenyl]-3-(pyridin-4-yl)pyrazol-1-yl}phenyl)piperazine-1-carboxylate (4)

To a mixture of N-[5-chloro-2-fluoro-3-(4,4,5,5-tetramethyl-1,3,2-dioxaborolan-2-yl)phenyl]butane-2-sulfonamide (650 mg, 1.659 mmol, 1 equiv) and tert-butyl 4-{4-[4-bromo-3-(pyridin-4-yl)pyrazol-1-yl]phenyl}piperazine-1-carboxylate (804 mg, 1.659 mmol, 1 equiv) in dioxane (10 mL) was added Pd(AMPHOS)₂Cl₂ (118 mg, 0.166 mmol, 0.1 equiv) and CsF (504 mg, 3.318 mmol, 2 equiv) in H₂O (2 mL). The resulting mixture was stirred at 90° C. for one hour under a nitrogen atmosphere. The resulting mixture was then concentrated under reduced pressure. The residue was purified by silica gel column chromatography eluted with CH₂Cl₂:EtOAc (1:1) to afford tert-butyl 4-(4-{4-[3-(butane-2-sulfonamido)-5-chloro-2-fluorophenyl]-3-(pyridin-4-yl)pyrazol-1-yl}phenyl)piperazine-1-carboxylate (250 mg, 20.94%) as an off-white solid. LCMS: C₃₃H₃₈ClFN₆O₄S requires: 668.2, found: m/z=669.1 [M+H]⁺.

### Step-4: Synthesis of rac-(2R)—N-(5-chloro-2-fluoro-3-{1-[4-(piperazin-1-yl)phenyl]-3-(pyridin-4-yl)pyrazol-4-yl}phenyl)butane-2-sulfonamide Trifluoroacetic Acid Salt (5)

A mixture of tert-butyl 4-(4-{4-[3-(butane-2-sulfonamido)-5-chloro-2-fluorophenyl]-3-(pyridin-4-yl)pyrazol-1-yl}phenyl)piperazine-1-carboxylate (250 mg, 0.374 mmol, 1 equiv) in TFA (3 mL) was stirred at 0° C. for one hour. The resulting mixture was then concentrated under reduced pressure and lyophilized. This resulted in rac-(2R)—N-(5-chloro-2-fluoro-3-{1-[4-(piperazin-1-yl)phenyl]-3-(pyridin-4-yl)pyrazol-4-yl}phenyl)butane-2-sulfonamide trifluoroacetic acid salt (151.9 mg, crude) as a yellow solid. LCMS: C₂₈H₃₀ClFN₆O₂S requires: 568.1, found: m/z=569.2 [M+H]⁺. ¹H NMR (400 MHz, Methanol-d₄) δ 8.79-8.73 (m, 2H), 8.63-8.58 (m, 1H), 8.18-8.11 (m, 2H), 7.94-7.86 (m, 2H), 7.70-7.63 (m, 1H), 7.45-7.38 (m, 1H), 7.28-7.20 (m, 2H), 3.57-3.50 (m, 4H), 3.50-3.40 (m, 4H), 3.17-3.05 (m, 1H), 2.13-1.98 (m, 1H), 1.68-1.52 (m, 1H), 1.40-1.31 (m, 3H), 1.07-0.99 (m, 3H).

### Synthesis of N-(5-chloro-2-fluoro-3-{1-[4-(piperazin-1-yl)phenyl]-3-(pyridin-4-yl)pyrazol-4-yl}phenyl)-3-fluoroazetidine-1-sulfonamide Trifluoroacetic Acid Salt (6)

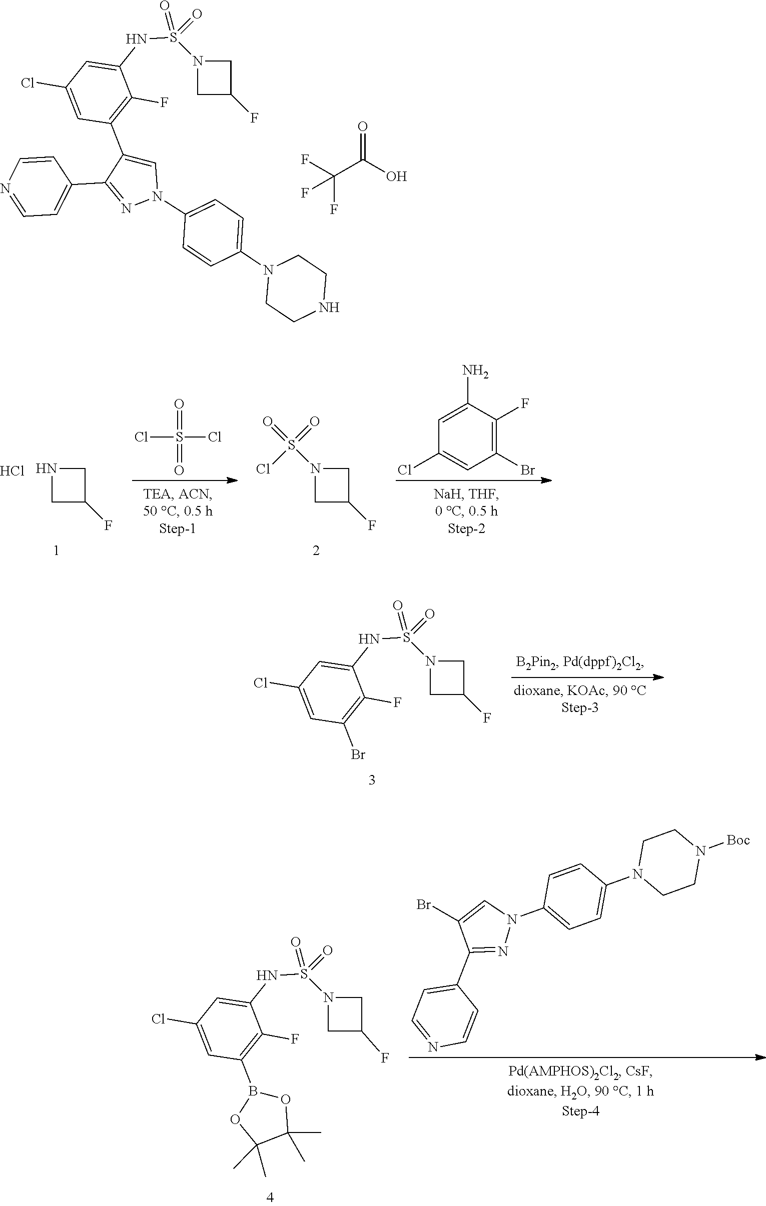

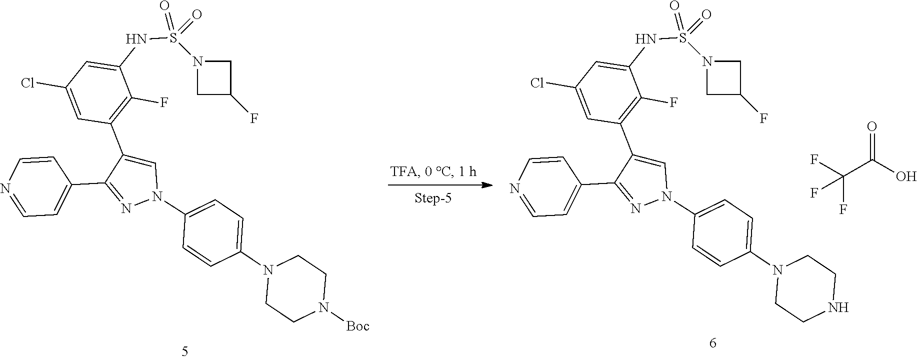 [SMILES: CC(C)(C)OC(=O)N1CCN(c2ccc(-n3cc(-c4cc(Cl)cc(NS(=O)(=O)N5CC(F)C5)c4F)c(-c4ccncc4)n3)cc2)CC1 ; O=S(=O)(Nc1cc(Cl)cc(-c2cn(-c3ccc(N4CCNCC4)cc3)nc2-c2ccncc2)c1F)N1CC(F)C1 ; O=C(O)C(F)(F)F]

### Step-1: Synthesis of 3-fluoroazetidine-1-sulfonyl Chloride (2)

To a mixture of sulfonyl chloride (9.0 g, 67.240 mmol, 2.5 equiv) in ACN (60 mL) was added 3-fluoroazetidine hydrochloride (3 g, 26.896 mmol, 1 equiv) and TEA (7.2 g, 71.274 mmol, 2.65 equiv) at 0° C. The resulting mixture was stirred at 50° C. for 30 min. The resulting mixture was then filtered. The filtrate was concentrated under reduced pressure. This resulted in 3-fluoroazetidine-1-sulfonyl chloride (3 g, crude) as an off-white solid. LCMS: C₃H₅ClFNO₂S requires: 172.9, found: m/z=173.8 [M+H]⁺.

### Step-2: Synthesis of N-(3-bromo-5-chloro-2-fluorophenyl)-3-fluoroazetidine-1-sulfonamide (3)

To a mixture of NaH (400 mg, 16.706 mmol, 2 equiv) in THF (100 mL) was added 3-bromo-5-chloro-2-fluoroaniline (1.9 g, 8.353 mmol, 1 equiv) at 0° C. under nitrogen atmosphere. The resulting mixture was stirred at 0° C. for 30 min under a nitrogen atmosphere. To the above mixture was added 3-fluoroazetidine-1-sulfonyl chloride (2.9 g, 16.706 mmol, 2 equiv) at 0° C. under a nitrogen atmosphere. The resulting mixture was then stirred at 50° C. overnight under a nitrogen atmosphere. The reaction was then quenched with saturated aqueous NH₄Cl at 0° C. The resulting mixture was concentrated under reduced pressure. The residue was purified by silica gel column chromatography eluted with PE:EtOAc (3:1) to afford N-(3-bromo-5-chloro-2-fluorophenyl)-3-fluoroazetidine-1-sulfonamide (500 mg, 13.24%) as a brown solid. LCMS: C₉H₈BrClF₂N₂O₂S requires: 359.9, found: m/z=358.9 [M−H]⁻.

### Step-3: Synthesis of N-[5-chloro-2-fluoro-3-(4,4,5,5-tetramethyl-1,3,2-dioxaborolan-2-yl)phenyl]-3-fluoroazetidine-1-sulfonamide (4)

To a mixture of N-(3-bromo-5-chloro-2-fluorophenyl)-3-fluoroazetidine-1-sulfonamide (450 mg, 1.245 mmol, 1 equiv) and 4,4,5,5-tetramethyl-2-(4,4,5,5-tetramethyl-1,3,2-dioxaborolan-2-yl)-1,3,2-dioxaborolane (B₂Pin₂) (632 mg, 2.490 mmol, 2 equiv) in dioxane (10 mL) was added KOAc (244 mg, 2.490 mmol, 2 equiv) and Pd(dppf)Cl₂ (101 mg, 0.125 mmol, 0.1 equiv). The resulting mixture was stirred at 90° C. overnight under a nitrogen atmosphere. The resulting mixture was then filtered. The filtrate was concentrated under reduced pressure. This resulted in N-[5-chloro-2-fluoro-3-(4,4,5,5-tetramethyl-1,3,2-dioxaborolan-2-yl)phenyl]-3-fluoroazetidine-1-sulfonamide (450 mg, crude) as a black oil. LCMS: C₁₅H₂₀BClF₂N₂O₄S requires: 408.1, found: m/z=409.2 [M+H]⁺.

### Step-4: Synthesis of tert-butyl 4-[4-(4-{5-chloro-2-fluoro-3-[(3-fluoroazetidin-1-ylsulfonyl)amino]phenyl}-3-(pyridin-4-yl)pyrazol-1-yl)phenyl]piperazine-1-carboxylate (5)

To a mixture of N-[5-chloro-2-fluoro-3-(4,4,5,5-tetramethyl-1,3,2-dioxaborolan-2-yl)phenyl]-3-fluoroazetidine-1-sulfonamide (450 mg, 1.101 mmol, 3 equiv) and tert-butyl 4-{4-[4-bromo-3-(pyridin-4-yl)pyrazol-1-yl]phenyl}piperazine-1-carboxylate (178 mg, 0.367 mmol, 1 equiv) in dioxane (5 mL) was added CsF (112 mg, 0.734 mmol, 2 equiv) in H₂O (1 mL) and Pd(AMPHOS)₂Cl₂ (26 mg, 0.037 mmol, 0.1 equiv). The resulting mixture was stirred at 90° C. for one hour under a nitrogen atmosphere. The resulting mixture was then concentrated under reduced pressure. The residue was purified by silica gel column chromatography eluted with CH₂Cl₂:EtOAc (3:2) to afford tert-butyl 4-[4-(4-{5-chloro-2-fluoro-3-[(3-fluoroazetidin-1-ylsulfonyl)amino]phenyl}-3-(pyridin-4-yl)pyrazol-1-yl)phenyl]piperazine-1-carboxylate (380 mg, 90.52%) as a black solid. LCMS: C₃₂H₃₄ClF₂N₇O₄S requires: 685.2, found: m/z=686.2 [M+H]⁺.

### Step-5: Synthesis of N-(5-chloro-2-fluoro-3-{1-[4-(piperazin-1-yl)phenyl]-3-(pyridin-4-yl)pyrazol-4-yl}phenyl)-3-fluoroazetidine-1-sulfonamide Trifluoroacetic Acid Salt (6)

A mixture of tert-butyl 4-[4-(4-{5-chloro-2-fluoro-3-[(3-fluoroazetidin-1-ylsulfonyl)amino]phenyl}-3-(pyridin-4-yl)pyrazol-1-yl)phenyl]piperazine-1-carboxylate (380 mg, 0.554 mmol, 1 equiv) in TFA (4 mL) was stirred at 0° C. for one hour. The resulting mixture was then concentrated under reduced pressure. The crude product was purified by prep-HPLC with the following conditions: Column: XSelect CSH Fluoro Phenyl 30×150 mm, 5 μm; Mobile Phase A: Water (0.05% TFA), Mobile Phase B: ACN; Flow rate: 60 mL/min; Gradient: 5% B to 40% B in 12 min; Wave Length: 254/220 nm to afford N-(5-chloro-2-fluoro-3-{1-[4-(piperazin-1-yl)phenyl]-3-(pyridin-4-yl)pyrazol-4-yl}phenyl)-3-fluoroazetidine-1-sulfonamide trifluoroacetic acid salt (220.8 mg, 21.01%) as a yellow solid. LCMS: C₂₇H₂₆ClF₂N₇O₂S requires: 585.2, found: m/z=586.1 [M+H]⁺. ¹H NMR (400 MHz, Methanol-d₄) δ 8.74-8.68 (m, 2H), 8.59-8.54 (m, 1H), 8.05-7.99 (m, 2H), 7.94-7.84 (m, 2H), 7.68-7.61 (m, 1H), 7.42-7.35 (m, 1H), 7.27-7.19 (m, 2H), 5.40-5.30 (m, 1H), 4.24-4.11 (m, 2H), 4.08-3.94 (m, 2H), 3.57-3.50 (m, 4H), 3.50-3.40 (m, 4H).

### Synthesis of ethylmethyl[(2,5-difluoro-3-{1-[4-(piperazin-1-yl)phenyl]-3-(pyridin-4-yl)pyrazol-4-yl}phenyl)sulfamoyl]amine Trifluoroacetic Acid Salt (5)

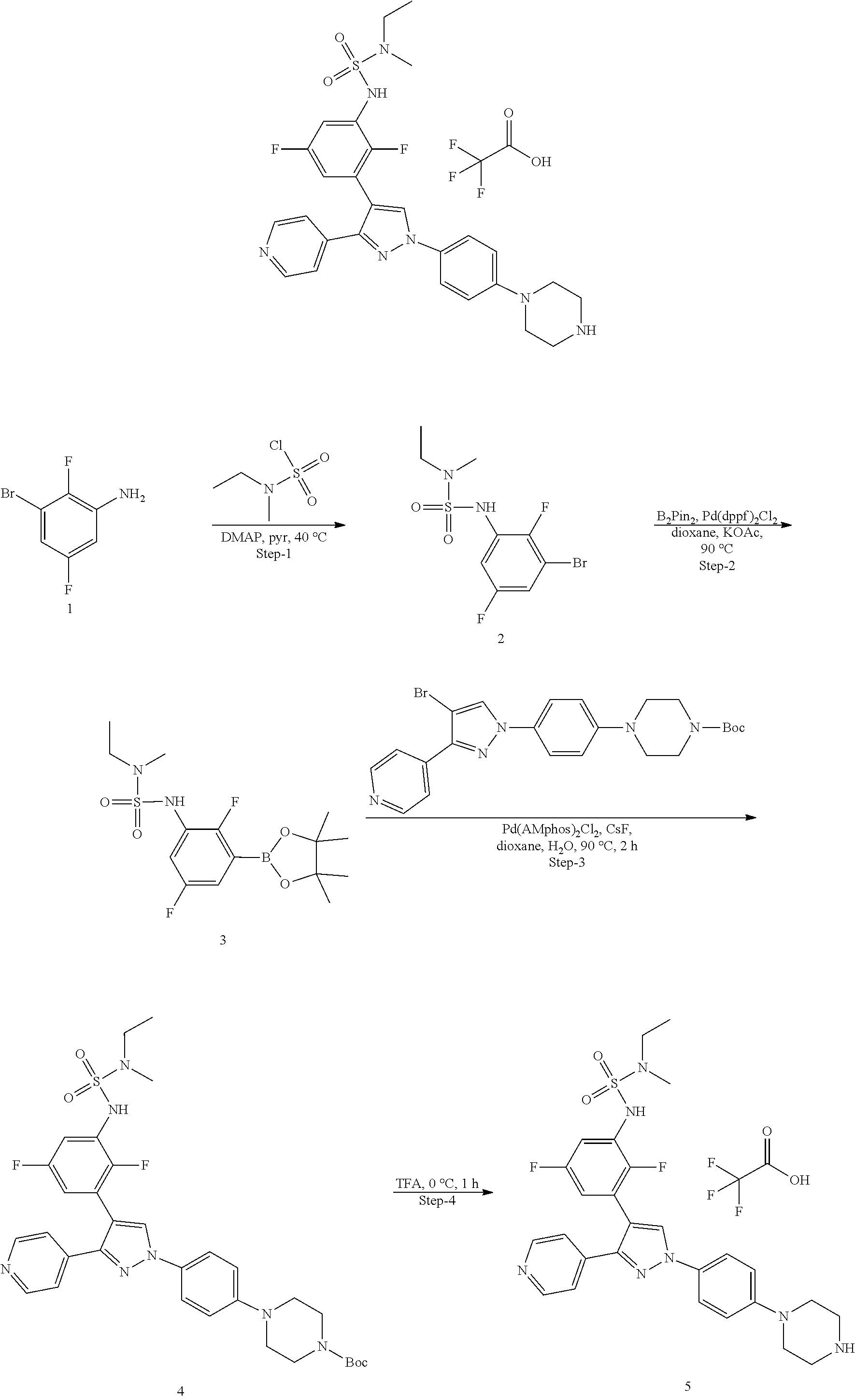

### Step-1: Synthesis of [(3-bromo-2,5-difluorophenyl)sulfamoyl](ethyl)methylamine (2)

To a mixture of 3-bromo-2,5-difluoroaniline (1.5 g, 7.211 mmol, 1 equiv) and N-ethyl-N-methylsulfamoyl chloride (2.84 g, 18.027 mmol, 2.5 equiv) in pyridine (2 mL) was added DMAP (1.06 g, 8.653 mmol, 1.2 equiv). The resulting mixture was stirred at 40° C. overnight under a nitrogen atmosphere. The resulting mixture was then concentrated under reduced pressure. The residue was purified by silica gel column chromatography eluted with PE:EtOAc (5:1) to afford [(3-bromo-2,5-difluorophenyl)sulfamoyl](ethyl)methylamine (1.4 g, 58.98%) as a brown yellow solid. LCMS: C₉H₁₁BrF₂N₂O₂S requires: 328.0, found: m/z=329.1 [M+H]⁺.

### Step-2: Synthesis of ethylmethyl{[2,5-difluoro-3-(4,4,5,5-tetramethyl-1,3,2-dioxaborolan-2-yl)phenyl]sulfamoyl}amine (3)

To a mixture of [(3-bromo-2,5-difluorophenyl)sulfamoyl](ethyl)methylamine (700 mg, 2.127 mmol, 1 equiv) and 4,4,5,5-tetramethyl-2-(4,4,5,5-tetramethyl-1,3,2-dioxaborolan-2-yl)-1,3,2-dioxaborolane (B₂Pin₂) (1.08 g, 4.254 mmol, 2 equiv) in 1,4-dioxane (10 mL) was added Pd(dppf)Cl₂ (156 mg, 0.213 mmol, 0.1 equiv) and KOAc (417 mg, 4.254 mmol, 2 equiv). The resulting mixture was stirred at 90° C. overnight under a nitrogen atmosphere. The resulting mixture was then filtered. The filtrate was concentrated under reduced pressure. This resulted in ethylmethyl{[2,5-difluoro-3-(4,4,5,5-tetramethyl-1,3,2-dioxaborolan-2-yl)phenyl]sulfamoyl}amine (700 mg, crude) as a black oil. LCMS: C₁₅H₂₃BF₂N₂O₄S requires: 376.1, found: m/z=377.2 [M+H]⁺.

### Step-3: Synthesis of tert-butyl 4-{4-[4-(3-{[ethyl(methyl)sulfamoyl]amino}-2,5-difluorophenyl)-3-(pyridin-4-yl)pyrazol-1-yl]phenyl}piperazine-1-carboxylate (4)

To a mixture of ethylmethyl{[2,5-difluoro-3-(4,4,5,5-tetramethyl-1,3,2-dioxaborolan-2-yl)phenyl]sulfamoyl}amine_(649 mg, 1.724 mmol, 1 equiv) and tert-butyl 4-{4-[4-bromo-3-(pyridin-4-yl)pyrazol-1-yl]phenyl}piperazine-1-carboxylate (835 mg, 1.724 mmol, 1 equiv) in dioxane (25 mL) was added CsF (524 mg, 3.448 mmol, 2 equiv) in H₂O (5 mL) and Pd(AMPHOS)₂Cl₂ (122 mg, 0.172 mmol, 0.1 equiv). The resulting mixture was stirred at 90° C. for 2 h under a nitrogen atmosphere. The resulting mixture was then concentrated under vacuum. The residue was purified by silica gel column chromatography eluted with DCM:EtOAc (2:3) to afford tert-butyl 4-{4-[4-(3-{[ethyl(methyl)sulfamoyl]amino}-2,5-difluorophenyl)-3-(pyridin-4-yl)pyrazol-1-yl]phenyl}piperazine-1-carboxylate (0.55 g, 48.81%) as a yellow solid. LCMS: C₃₂H₃₇F₂N₇O₄S requires: 653.3, found: m/z=654.3 [M+H]⁺.

### Step-4: Synthesis of ethylmethyl[(2,5-difluoro-3-{1-[4-(piperazin-1-yl)phenyl]-3-(pyridin-4-yl)pyrazol-4-yl}phenyl)sulfamoyl]amine Trifluoroacetic Acid Salt

A mixture of tert-butyl 4-{4-[4-(3-{[ethyl(methyl)sulfamoyl]amino}-2,5-difluorophenyl)-3-(pyridin-4-yl)pyrazol-1-yl]phenyl}piperazine-1-carboxylate (0.55 g, 0.841 mmol, 1 equiv) in TFA (5 mL) was stirred at 0° C. for one hour. The resulting mixture was then concentrated under reduced pressure and lyophilized. This resulted in ethylmethyl[(2,5-difluoro-3-{1-[4-(piperazin-1-yl)phenyl]-3-(pyridin-4-yl)pyrazol-4-yl}phenyl)sulfamoyl]amine trifluoroacetic acid salt (301.9 mg, crude) as a yellow solid. LCMS: C₂₇H₂₉F₂N₇O₂S requires: 553.2, found: m/z=554.2 [M+H]⁺. ¹H NMR (400 MHz, Methanol-d₄) δ 8.79-8.73 (m, 2H), 8.62-8.57 (m, 1H), 8.18-8.12 (m, 2H), 7.95-7.86 (m, 2H), 7.42-7.32 (m, 1H), 7.28-7.20 (m, 2H), 7.14-7.05 (m, 1H), 3.57-3.50 (m, 4H), 3.47-3.40 (m, 4H), 3.33-3.23 (m, 2H), 2.87-2.83 (m, 3H), 1.20-1.11 (m, 3H)

### Synthesis of ethylmethyl[(5-chloro-2-fluoro-3-{1-[5-(piperazin-1-yl)pyridin-2-yl]-3-(pyridin-4-yl)pyrazol-4-yl}phenyl)sulfamoyl]amine Trifluoroacetic Acid Salt (3)

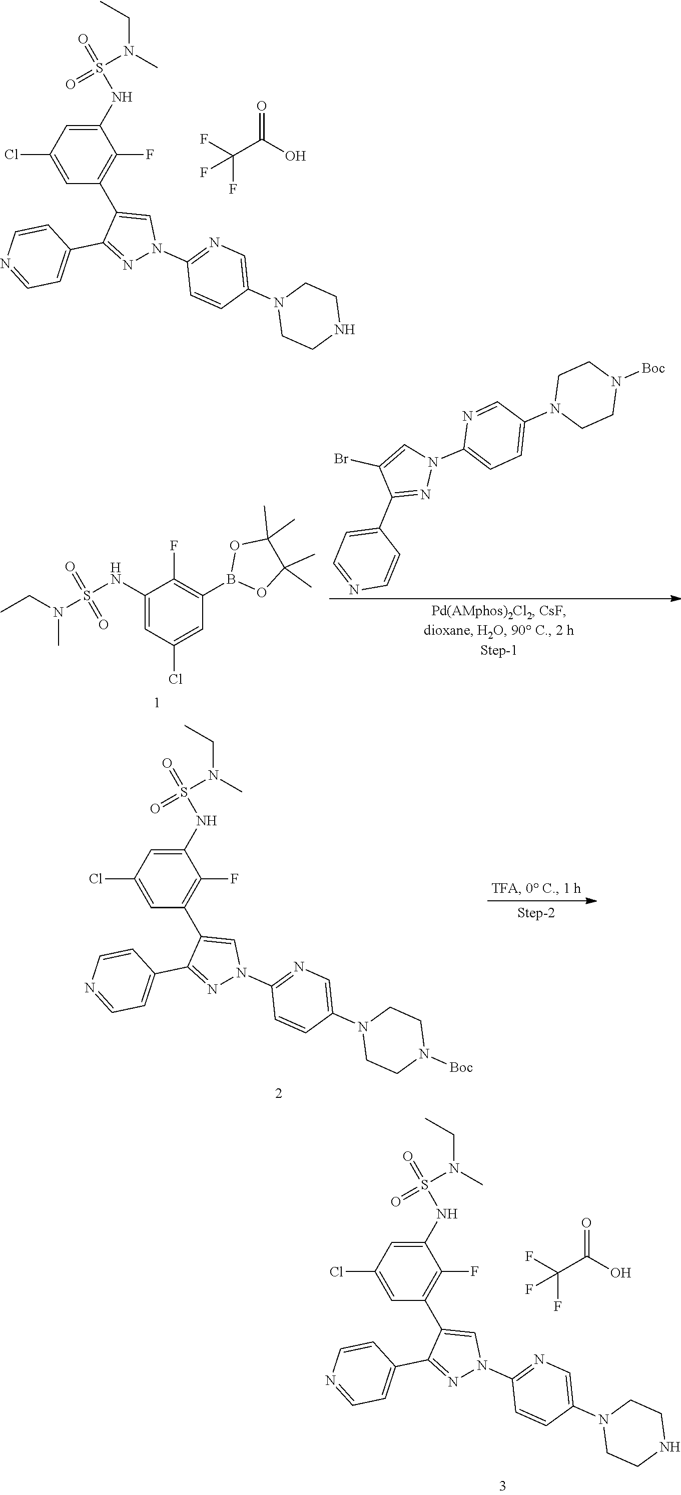

### Step-1: Synthesis of tert-butyl 4-{6-[4-(5-chloro-3-{[ethyl(methyl)sulfamoyl]amino}-2-fluorophenyl)-3-(pyridin-4-yl)pyrazol-1-yl]pyridin-3-yl}piperazine-1-carboxylate (2)

To a mixture of ethylmethyl{[5-chloro-2-fluoro-3-(4,4,5,5-tetramethyl-1,3,2-dioxaborolan-2-yl)phenyl]sulfamoyl}amine (1 g, 2.547 mmol, 1 equiv) and tert-butyl 4-{6-[4-bromo-3-(pyridin-4-yl)pyrazol-1-yl]pyridin-3-yl}piperazine-1-carboxylate (1.2 g, 2.547 mmol, 1 equiv) in dioxane (20 mL) was added Pd(AMphos)₂Cl₂ (180 mg, 0.255 mmol, 0.1 equiv) and CsF (774 mg, 5.094 mmol, 2 equiv) in H₂O (4 mL). The resulting mixture was stirred at 90° C. for 2 h under a nitrogen atmosphere. The resulting mixture was then concentrated under reduced pressure. The residue was purified by silica gel column chromatography eluted with CH₂Cl₂:EtOAc (1:1) to afford tert-butyl 4-{6-[4-(5-chloro-3-{[ethyl(methyl)sulfamoyl]amino}-2-fluorophenyl)-3-(pyridin-4-yl)pyrazol-1-yl]pyridin-3-yl}piperazine-1-carboxylate (500 mg, 23.40%) as a brown semi-solid. LCMS: C₃₁H₃₆ClFN₈O₄S requires: 670.2, found: m/z=671.3 [M+H]⁺.

### Step-2: Synthesis of ethylmethyl[(5-chloro-2-fluoro-3-{1-[5-(piperazin-1-yl)pyridin-2-yl]-3-(pyridin-4-yl)pyrazol-4-yl}phenyl)sulfamoyl]amine Trifluoroacetic Acid Salt (3)

A mixture of tert-butyl 4-{6-[4-(5-chloro-3-{[ethyl(methyl)sulfamoyl]amino}-2-fluorophenyl)-3-(pyridin-4-yl)pyrazol-1-yl]pyridin-3-yl}piperazine-1-carboxylate (500 mg, 0.745 mmol, 1 equiv) in TFA (5 mL) was stirred for one hour at 0° C. The resulting mixture was then concentrated under reduced pressure. The crude product was purified by prep-HPLC with the following conditions: Column: XBridge Prep Phenyl OBD Column 19×250 mm; Mobile Phase A: Water (0.05% TFA), Mobile Phase B: MeOH; Flow rate: 25 mL/min; Gradient: 40% B to 55% B in 10 min; Wave Length: 254/220 nm to afford ethylmethyl[(5-chloro-2-fluoro-3-{1-[5-(piperazin-1-yl)pyridin-2-yl]-3-(pyridin-4-yl)pyrazol-4-yl}phenyl)sulfamoyl]amine trifluoroacetic acid salt (222.9 mg, 43.50%) as a yellow solid. LCMS: C₂₆H₂₈ClFN₈O₂S requires: 570.1, found: m/z=571.2 [M+H]⁺. ¹HNMR (400 MHz, Methanol-d₄) δ 8.88-8.84 (m, 1H), 8.81-8.75 (m, 2H), 8.33-8.27 (m, 1H), 8.21-8.07 (m, 3H), 7.77-7.70 (m, 1H), 7.64-7.57 (m, 1H), 7.37-7.31 (m, 1H), 3.67-3.56 (m, 4H), 3.50-3.42 (m, 4H), 3.33-3.23 (m, 2H), 2.87-2.83 (m, 3H), 1.21-1.12 (m, 3H).

### Synthesis of (3R)—N-(5-chloro-2-fluoro-3-{1-[2-fluoro-4-(piperazin-1-yl)phenyl]-3-(pyridin-4-yl)pyrazol-4-yl}phenyl)-3-fluoropyrrolidine-1-sulfonamide Trifluoroacetic Acid Salt (3)

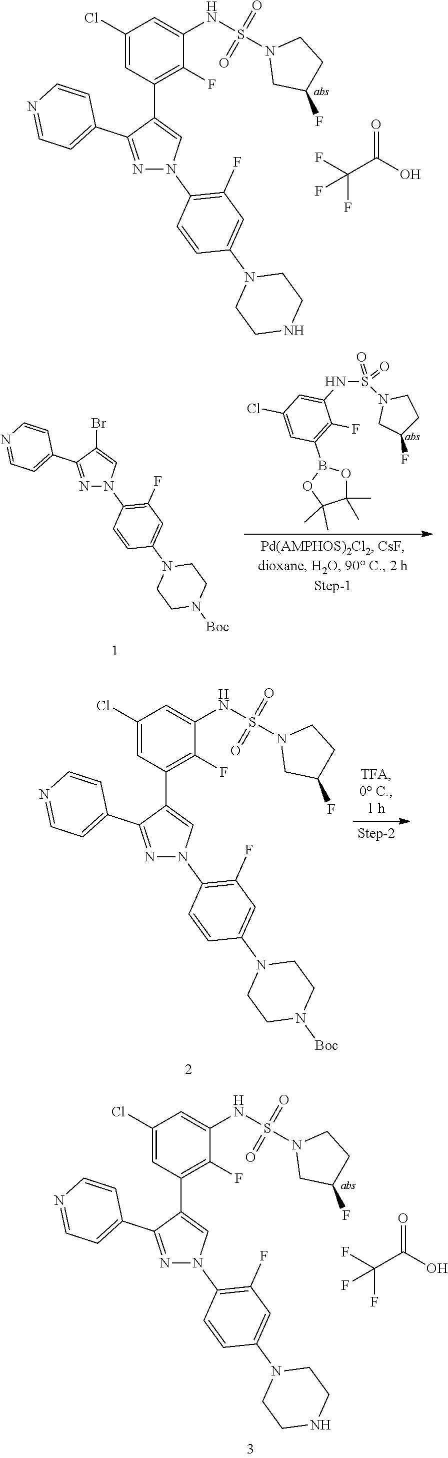

### Step-1: Synthesis of tert-butyl 4-{4-[4-(5-chloro-2-fluoro-3-{[(3R)-3-fluoropyrrolidin-1-ylsulfonyl]amino}phenyl)-3-(pyridin-4-yl)pyrazol-1-yl]-3-fluorophenyl}piperazine-1-carboxylate (2)

To a mixture of tert-butyl 4-{4-[4-bromo-3-(pyridin-4-yl)pyrazol-1-yl]-3-fluorophenyl}piperazine-1-carboxylate (1.5 g, 2.986 mmol, 1 equiv) and (3R)—N-[5-chloro-2-fluoro-3-(4,4,5,5-tetramethyl-1,3,2-dioxaborolan-2-yl)phenyl]-3-fluoropyrrolidine-1-sulfonamide (1.3 g, 2.986 mmol, 1 equiv) in dioxane (20 mL) was added Pd(AMPHOS)₂Cl₂ (211 mg, 0.299 mmol, 0.1 equiv) and CsF (907 mg, 5.972 mmol, 2 equiv) in H₂O (4 mL). The resulting mixture was stirred at 90° C. for 2 h under a nitrogen atmosphere. The resulting mixture was then concentrated under reduced pressure. The residue was purified by silica gel column chromatography eluted with CH₂Cl₂:EtOAc (1:1) to afford tert-butyl 4-{4-[4-(5-chloro-2-fluoro-3-{[(3R)-3-fluoropyrrolidin-1-ylsulfonyl]amino}phenyl)-3-(pyridin-4-yl)pyrazol-1-yl]-3-fluorophenyl}piperazine-1-carboxylate (900 mg, 31.48%) as a brown semi-solid. LCMS: C₃₃H₃₅ClF₃N₇O₄S requires: 717.2, found: m/z=718.3 [M+H]⁺.

### Step-2: Synthesis of (3R)—N-(5-chloro-2-fluoro-3-{1-[2-fluoro-4-(piperazin-1-yl)phenyl]-3-(pyridin-4-yl)pyrazol-4-yl}phenyl)-3-fluoropyrrolidine-1-sulfonamide Trifluoroacetic Acid (3)

A mixture of tert-butyl 4-{4-[4-(5-chloro-2-fluoro-3-{[(3R)-3-fluoropyrrolidin-1-ylsulfonyl]amino}phenyl)-3-(pyridin-4-yl)pyrazol-1-yl]-3-fluorophenyl}piperazine-1-carboxylate (900 mg, 1.253 mmol, 1 equiv) in TFA (5 mL) was stirred at 0° C. for one hour. The resulting mixture was then concentrated under reduced pressure. The crude product was purified by reverse phase flash chromatography with the following conditions: Column: Xselect CSH C18 OBD Column 30×150 mm, 5 μm; Mobile Phase A: Water (0.05% TFA), Mobile Phase B: ACN; Flow rate: 60 mL/min; Gradient: 5% B to 5% B in 2 min, 10% B to 35% B in 15 min; Wave Length: 254/220 nm to afford (3R)—N-(5-chloro-2-fluoro-3-{1-[2-fluoro-4-(piperazin-1-yl)phenyl]-3-(pyridin-4-yl)pyrazol-4-yl}phenyl)-3-fluoropyrrolidine-1-sulfonamide trifluoroacetic acid salt (485.3 mg, 51.84%) as a yellow solid. LCMS: C₂₈H₂₇ClF₃N₇O₂S requires: 617.1, found: m/z=618.1 [M+H]⁺. ¹HNMR (300 MHz, Methanol-d₄) δ 8.77-8.69 (m, 2H), 8.39-8.32 (m, 1H), 8.11-8.00 (m, 2H), 7.90-7.78 (m, 1H), 7.73-7.64 (m, 1H), 7.36-7.27 (m, 1H), 7.15-7.00 (m, 2H), 5.48-5.06 (m, 1H), 3.68-3.49 (m, 6H), 3.53-3.38 (m, 6H), 2.41-1.96 (m, 2H).

### Synthesis of N-(5-chloro-2-fluoro-3-{1-[2-fluoro-4-(piperazin-1-yl)phenyl]-3-(pyridin-4-yl)pyrazol-4-yl}phenyl)-3-fluoroazetidine-1-sulfonamide Trifluoroacetic Acid Salt (3)

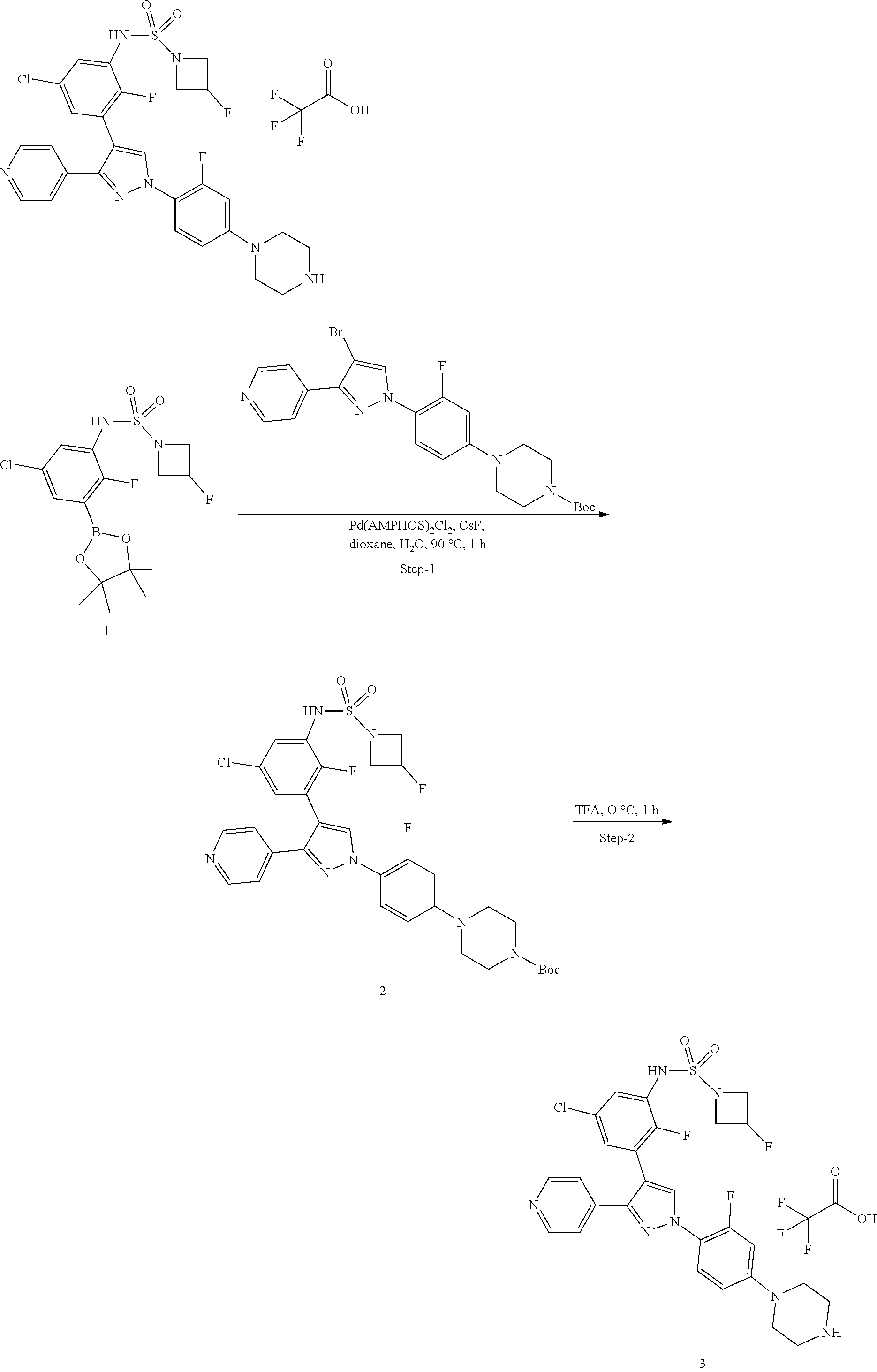

### Step-1: Synthesis of tert-butyl 4-[4-(4-{5-chloro-2-fluoro-3-[(3-fluoroazetidin-1-ylsulfonyl)amino]phenyl}-3-(pyridin-4-yl)pyrazol-1-yl)-3-fluorophenyl]piperazine-1-carboxylate (2)

To a mixture of tert-butyl 4-{4-[4-bromo-3-(pyridin-4-yl)pyrazol-1-yl]-3-fluorophenyl}piperazine-1-carboxylate (830 mg, 1.652 mmol, 1 equiv) in dioxane (10 mL) was added N-[5-chloro-2-fluoro-3-(4,4,5,5-tetramethyl-1,3,2-dioxaborolan-2-yl)phenyl]-3-fluoroazetidine-1-sulfonamide (2.1 g, 4.956 mmol, 3 equiv), CsF (502 mg, 3.304 mmol, 2 equiv) in H₂O (2 mL), and Pd(AMPHOS)₂Cl₂ (117 mg, 0.165 mmol, 0.1 equiv) at room temperature. The resulting mixture was stirred at 90° C. for one hour under a nitrogen atmosphere. The resulting mixture was then concentrated under reduced pressure. The residue was purified by silica gel column chromatography eluted with PE:EtOAc (1:2) to afford tert-butyl 4-[4-(4-{5-chloro-2-fluoro-3-[(3-fluoroazetidin-1-ylsulfonyl)amino]phenyl}-3-(pyridin-4-yl)pyrazol-1-yl)-3-fluorophenyl]piperazine-1-carboxylate (550 mg, 41.60%) as a yellow solid. LCMS: C₃₂H₃₃ClF₃N₇O₄S requires: 703.2, found: m/z=704.2 [M+H]⁺.

### Step-2: Synthesis of N-(5-chloro-2-fluoro-3-{1-[2-fluoro-4-(piperazin-1-yl)phenyl]-3-(pyridin-4-yl)pyrazol-4-yl}phenyl)-3-fluoroazetidine-1-sulfonamide Trifluoroacetic Acid Salt (3)

A mixture of tert-butyl 4-[4-(4-{5-chloro-2-fluoro-3-[(3-fluoroazetidin-1-ylsulfonyl)amino]phenyl}-3-(pyridin-4-yl)pyrazol-1-yl)-3-fluorophenyl]piperazine-1-carboxylate (500 mg, 0.710 mmol, 1 equiv) in TFA (5 mL) was stirred at 0° C. for one hour. The resulting mixture was then concentrated under reduced pressure. The crude product was purified by prep-HPLC with the following conditions: Column: XSelect CSH Fluoro Phenyl 30×150 mm, 5 μm; Mobile Phase A: Water (0.05% TFA), Mobile Phase B: ACN; Flow rate: 60 mL/min; Gradient: 5% B to 40% B in 12 min; Wave Length: 254/220 nm to afford N-(5-chloro-2-fluoro-3-{1-[2-fluoro-4-(piperazin-1-yl)phenyl]-3-(pyridin-4-yl)pyrazol-4-yl}phenyl)-3-fluoroazetidine-1-sulfonamide trifluoroacetic acid salt (264.2 mg, 50.88%) as a yellow solid. LCMS: C₂₇H₂₅ClF₃N₇O₂S requires: 603.1, found: m/z=604.2 [M+H]⁺. ¹H NMR (300 MHz, Methanol-d₄) δ 8.81-8.72 (m, 2H), 8.41-8.37 (m, 1H), 8.10-8.05 (m, 2H), 7.90-7.79 (m, 1H), 7.69-7.63 (m, 1H), 7.40-7.33 (m, 1H), 7.14-7.01 (m, 2H), 5.42-5.14 (m, 1H), 4.25-4.10 (m, 2H), 4.09-3.91 (m, 2H), 3.67-3.54 (m, 4H), 3.48-3.38 (m, 4H).

### Synthesis of (3R)—N-(5-chloro-2-fluoro-3-{1-[3-fluoro-4-(piperazin-1-yl)phenyl]-3-(pyridin-4-yl)pyrazol-4-yl}phenyl)-3-fluoropyrrolidine-1-sulfonamide Trifluoroacetic Acid Salt (3)

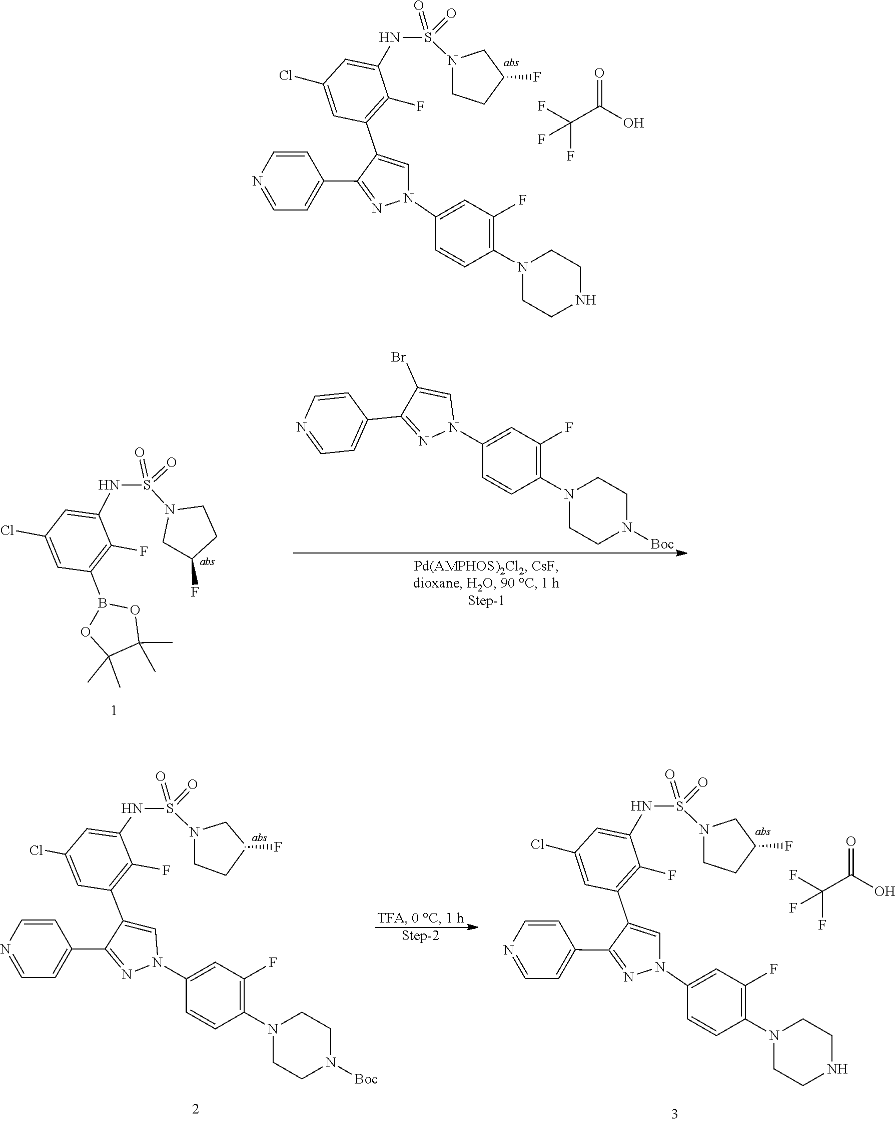

### Step-1: Synthesis of tert-butyl 4-{4-[4-(5-chloro-2-fluoro-3-{[(3R)-3-fluoropyrrolidin-1-ylsulfonyl]amino}phenyl)-3-(pyridin-4-yl)pyrazol-1-yl]-2-fluorophenyl}piperazine-1-carboxylate (2)

To a mixture of tert-butyl 4-{4-[4-bromo-3-(pyridin-4-yl)pyrazol-1-yl]-2-fluorophenyl}piperazine-1-carboxylate (900 mg, 1.791 mmol, 1 equiv) and (3R)—N-[5-chloro-2-fluoro-3-(4,4,5,5-tetramethyl-1,3,2-dioxaborolan-2-yl)phenyl]-3-fluoropyrrolidine-1-sulfonamide (757 mg, 1.791 mmol, 1 equiv) in dioxane (10 mL) was added CsF (544 mg, 3.582 mmol, 2 equiv) and Pd(AMPHOS)₂Cl₂ (127 mg, 0.179 mmol, 0.1 equiv) in H₂O (2 mL). The resulting mixture was stirred at 90° C. for one hour under a nitrogen atmosphere. The resulting mixture was then concentrated under reduced pressure. The residue was purified by silica gel column chromatography eluted with PE:EtOAc (1:1) to afford tert-butyl 4-{4-[4-(5-chloro-2-fluoro-3-{[(3R)-3-fluoropyrrolidin-1-ylsulfonyl]amino}phenyl)-3-(pyridin-4-yl)pyrazol-1-yl]-2-fluorophenyl}piperazine-1-carboxylate (660 mg, 51.30%) as a white solid. LCMS: C₃₃H₃₅ClF₃N₇O₄S requires: 717.2, found: m/z=718.3 [M+H]⁺.

### Step-2: Synthesis of (3R)—N-(5-chloro-2-fluoro-3-{1-[3-fluoro-4-(piperazin-1-yl)phenyl]-3-(pyridin-4-yl)pyrazol-4-yl}phenyl)-3-fluoropyrrolidine-1-sulfonamide Trifluoroacetic Acid Salt (3)

A mixture of tert-butyl 4-{4-[4-(5-chloro-2-fluoro-3-{[(3R)-3-fluoropyrrolidin-1-ylsulfonyl]amino}phenyl)-3-(pyridin-4-yl)pyrazol-1-yl]-2-fluorophenyl}piperazine-1-carboxylate (660 mg, 0.919 mmol, 1 equiv) in TFA (5 mL) was stirred at 0° C. for one hour. The resulting mixture was concentrated under reduced pressure. The crude product was purified by prep-HPLC with the following conditions: Column: XSelect CSH Prep C18 OBD Column, 30×150 mm, 5 μm; Mobile Phase A: Water (0.05% TFA), Mobile Phase B: ACN; Flow rate: 60 mL/min; Gradient: 5% B to 40% B in 12 min; Wave Length: 254/220 nm to provide (3R)—N-(5-chloro-2-fluoro-3-{1-[3-fluoro-4-(piperazin-1-yl)phenyl]-3-(pyridin-4-yl)pyrazol-4-yl}phenyl)-3-fluoropyrrolidine-1-sulfonamide trifluoroacetic acid salt (295.8 mg, 40.13%) as a yellow solid. LCMS: C₂₈H₂₇ClF₃N₇O₂S requires: 617.2, found: m/z=618.2 [M+H]⁺. ¹H NMR (300 MHz, Methanol-d₄) δ 8.83-8.68 (m, 2H), 8.64 (s, 1H), 8.18-8.05 (m, 2H), 7.90-7.74 (m, 2H), 7.74-7.65 (m, 1H), 7.41-7.26 (m, 2H), 5.41-5.14 (m, 1H), 3.63-3.38 (m, 12H), 2.33-1.93 (m, 2H).

### Synthesis of N-[5-(difluoromethyl)-2-fluoro-3-{1-[2-fluoro-4-(piperazin-1-yl)phenyl]-3-(pyridin-4-yl)pyrazol-4-yl}phenyl]pyrrolidine-1-sulfonamide Trifluoroacetic Acid Salt ((5)

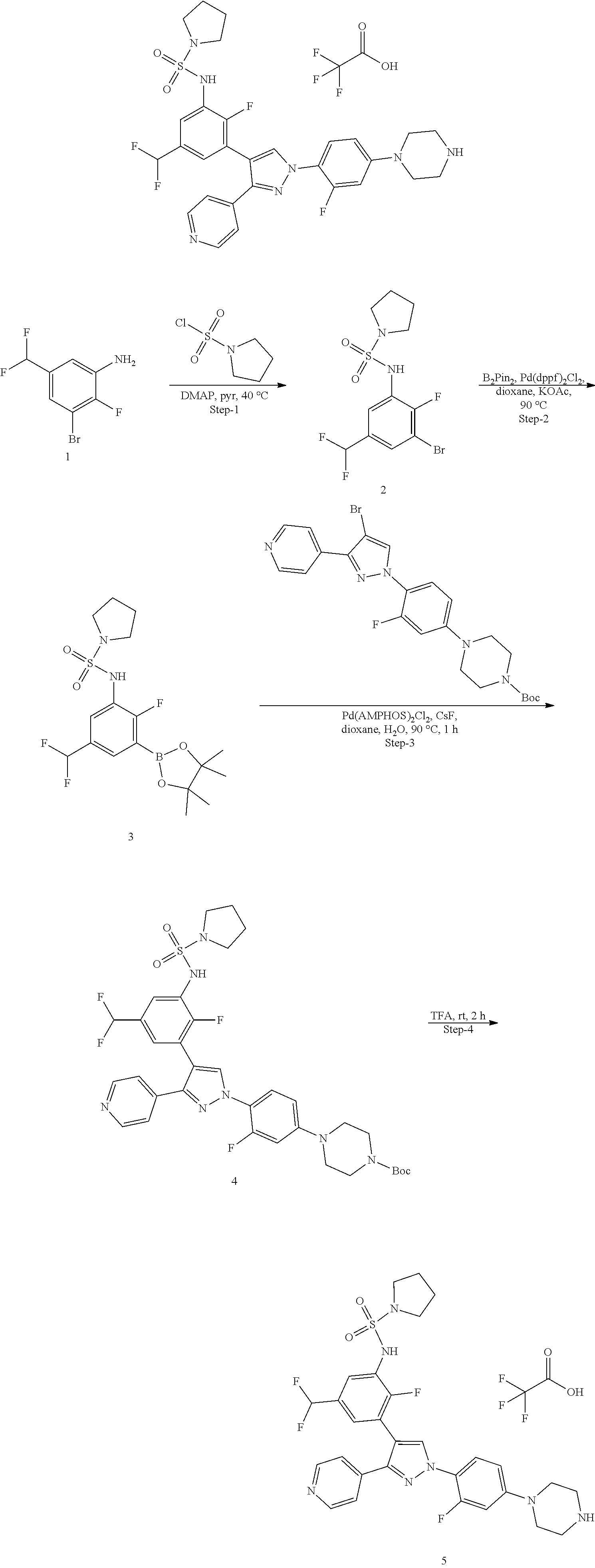

### Step-1: Synthesis of N-[3-bromo-5-(difluoromethyl)-2-fluorophenyl]pyrrolidine-1-sulfonamide (2)

To a mixture of 3-bromo-5-(difluoromethyl)-2-fluoroaniline (1.8 g, 7.499 mmol, 1 equiv) and pyrrolidine-1-sulfonyl chloride (2.3 g, 13.498 mmol, 1.8 equiv) in pyridine (3 mL) was added DMAP (1.1 g, 8.999 mmol, 1.2 equiv). The resulting mixture was stirred at 40° C. overnight under a nitrogen atmosphere. The resulting mixture was then concentrated under reduced pressure. The residue was purified by silica gel column chromatography eluted with PE:EtOAc (1:1) to afford N-[3-bromo-5-(difluoromethyl)-2-fluorophenyl]pyrrolidine-1-sulfonamide (1 g, 26.44%) as a yellow solid. LCMS: C₁₁H₁₂BrF₃N₂O₂S requires: 371.9, found: m/z=373.0 [M+H]⁺.

### Step-2: Synthesis of N-[5-(difluoromethyl)-2-fluoro-3-(4,4,5,5-tetramethyl-1,3,2-dioxaborolan-2-yl)phenyl]pyrrolidine-1-sulfonamide (3)

To a mixture of N-[3-bromo-5-(difluoromethyl)-2-fluorophenyl]pyrrolidine-1-sulfonamide (900 mg, 2.412 mmol, 1 equiv) and 4,4,5,5-tetramethyl-2-(4,4,5,5-tetramethyl-1,3,2-dioxaborolan-2-yl)-1,3,2-dioxaborolane (B₂Pin₂) (1225 mg, 4.824 mmol, 2 equiv) in dioxane (10 mL) was added KOAc (473 mg, 4.824 mmol, 2 equiv) and Pd(dppf)Cl₂ (197 mg, 0.241 mmol, 0.1 equiv). The resulting mixture was stirred at 90° C. overnight under a nitrogen atmosphere. The resulting mixture was then filtered. The resulting mixture was concentrated under reduced pressure to afford N-[5-(difluoromethyl)-2-fluoro-3-(4,4,5,5-tetramethyl-1,3,2-dioxaborolan-2-yl)phenyl]pyrrolidine-1-sulfonamide (900 mg, crude) as a black oil. LCMS: C11H₁₂BrF₃N₂O₂S requires: 420.2, found: m/z=421.1 [M+H]⁺.

### Step-3: Synthesis of tert-butyl 4-(4-{4-[5-(difluoromethyl)-2-fluoro-3-[(pyrrolidine-1-sulfonyl)amino]phenyl}-3-(pyridin-4-yl)pyrazol-1-yl]-3-fluorophenyl)piperazine-1-carboxylate (4)

To a mixture of N-[5-(difluoromethyl)-2-fluoro-3-(4,4,5,5-tetramethyl-1,3,2-dioxaborolan-2-yl)phenyl]pyrrolidine-1-sulfonamide (2.3 g, 5.572 mmol, 2.8 equiv) and tert-butyl 4-{4-[4-bromo-3-(pyridin-4-yl)pyrazol-1-yl]-3-fluorophenyl}piperazine-1-carboxylate (1 g, 1.990 mmol, 1 equiv) in 1,4-dioxane (40 mL) was added CsF (605 mg, 3.980 mmol, 2 equiv) in H₂O (8 mL) and Pd(AMPHOS)₂Cl₂ (141 mg, 0.199 mmol, 0.1 equiv). The resulting mixture was stirred at 90° C. for one hour under a nitrogen atmosphere. The resulting mixture was concentrated under reduced pressure. The residue was purified by silica gel column chromatography eluted with PE:EtOAc (1:1) to afford tert-butyl 4-(4-{4-[5-(difluoromethyl)-2-fluoro-3-[(pyrrolidine-1-sulfonyl)amino]phenyl]-3-(pyridin-4-yl)pyrazol-1-yl}-3-fluorophenyl)piperazine-1-carboxylate (560 mg, 39.31%) as a yellow solid. LCMS: C₃₄H₃₇F₄N₇O₄S requires: 715.3, found: m/z=716.1 [M+H]⁺.

### Step-4: Synthesis of N-[5-(difluoromethyl)-2-fluoro-3-{1-[2-fluoro-4-(piperazin-1-yl)phenyl]-3-(pyridin-4-yl)pyrazol-4-yl}phenyl]pyrrolidine-1-sulfonamide Trifluoroacetic Acid Salt (5)

A mixture of tert-butyl 4-(4-{4-[5-(difluoromethyl)-2-fluoro-3-[(pyrrolidine-1-sulfonyl)amino]phenyl]-3-(pyridin-4-yl)pyrazol-1-yl}-3-fluorophenyl)piperazine-1-carboxylate (470 mg, 0.657 mmol, 1 equiv) in TFA (7 mL) was stirred at room temperature for 2 h. The resulting mixture was then concentrated under reduced pressure. The crude product was purified by prep-HPLC with the following conditions: Column: Xcelect CSH F-pheny OBD Column 19×250 mm, 5 μm; Mobile Phase A: Water (0.05% TFA), Mobile Phase B: MeOH; Flow rate: 25 mL/min; Gradient: 28% B to 43% B in 30 min; Wave Length: 254/220 nm to provide N-[5-(difluoromethyl)-2-fluoro-3-{1-[2-fluoro-4-(piperazin-1-yl)phenyl]-3-(pyridin-4-yl)pyrazol-4-yl}phenyl]pyrrolidine-1-sulfonamide trifluoroacetic acid salt (293.2 mg, 61.19%) as a yellow solid. LCMS: C₂₉H₂₉F₄N₇O₂S requires: 615.2, found: m/z=616.2 [M+H]⁺. ¹H NMR (300 MHz, Methanol-d₄) δ 8.70-8.63 (m, 2H), 8.38-8.34 (m, 1H), 7.93-7.77 (m, 4H), 7.49-7.41 (m, 1H), 7.13-6.99 (m, 2H), 6.85-6.62 (m, 1H), 3.63-3.55 (m, 4H), 3.48-3.40 (m, 4H), 3.34 (s, 4H), 1.93-1.84 (m, 4H).

### Synthesis of N-(5-chloro-2-fluoro-3-{1-[3-fluoro-4-(piperazin-1-yl)phenyl]-3-(pyridin-4-yl)pyrazol-4-yl}phenyl)pyrrolidine-1-sulfonamide Trifluoroacetic Acid Salt

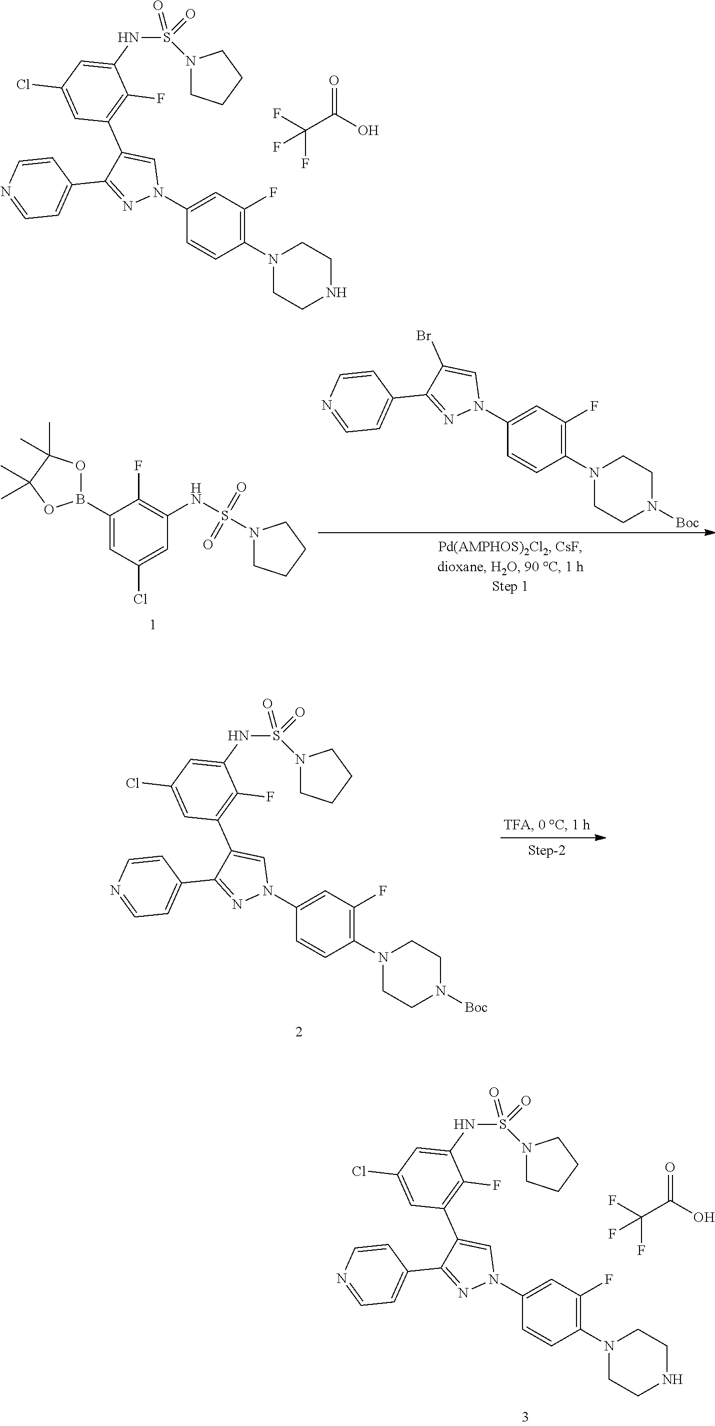

### Step-1: Synthesis of tert-butyl 4-[4-(4-{5-chloro-2-fluoro-3-[(pyrrolidine-1-sulfonyl)amino]phenyl}-3-(pyridin-4-yl)pyrazol-1-yl)-2-fluorophenyl]piperazine-1-carboxylate (2)

To a mixture of tert-butyl 4-{4-[4-bromo-3-(pyridin-4-yl)pyrazol-1-yl]-2-fluorophenyl}piperazine-1-carboxylate (1 g, 1.990 mmol, 1 equiv) and N-[5-chloro-2-fluoro-3-(4,4,5,5-tetramethyl-1,3,2-dioxaborolan-2-yl)phenyl]pyrrolidine-1-sulfonamide (805 mg, 1.990 mmol, 1 equiv) in dioxane (10 mL) was added CsF (604 mg, 3.980 mmol, 2 equiv) and Pd(AMPHOS)₂Cl₂ (141 mg, 0.199 mmol, 0.1 equiv) in H₂O (2 mL). The resulting mixture was stirred at 90° C. for one hour under a nitrogen atmosphere. The resulting mixture was then concentrated under reduced pressure. The residue was purified by silica gel column chromatography eluted with CH₂Cl₂:MeOH (1:1) to afford tert-butyl 4-[4-(4-{5-chloro-2-fluoro-3-[(pyrrolidine-1-sulfonyl)amino]phenyl}-3-(pyridin-4-yl)pyrazol-1-yl)-2-fluorophenyl]piperazine-1-carboxylate (700 mg, 40.18%) as a white solid. LCMS: C₃₃H₃₆ClF₂N₇O₄S requires: 699.2, found: m/z=700.2 [M+H]⁺.

### Step-2: Synthesis of N-(5-chloro-2-fluoro-3-{1-[3-fluoro-4-(piperazin-1-yl)phenyl]-3-(pyridin-4-yl)pyrazol-4-yl}phenyl)pyrrolidine-1-sulfonamide Trifluoroacetic Acid Salt (3)

A mixture of tert-butyl 4-[4-(4-{5-chloro-2-fluoro-3-[(pyrrolidine-1-sulfonyl)amino]phenyl}-3-(pyridin-4-yl)pyrazol-1-yl)-2-fluorophenyl]piperazine-1-carboxylate (700 mg, 1.000 mmol, 1 equiv) in TFA (7 mL) was stirred at 0° C. for one hour. The resulting mixture was then concentrated under reduced pressure. The crude product was purified by prep-HPLC with the following conditions: Column: XSelect CSH Prep C18 OBD Column, 19×250 mm, 5 μm; Mobile Phase A: Water (0.05% TFA), Mobile Phase B: MeOH; Flow rate: 25 mL/min; Gradient: 35% B to 50% B in 11 min; Wave Length: 254/220 nm to provide N-(5-chloro-2-fluoro-3-{1-[3-fluoro-4-(piperazin-1-yl)phenyl]-3-(pyridin-4-yl)pyrazol-4-yl}phenyl)pyrrolidine-1-sulfonamide trifluoroacetic acid salt (381.0 mg, 52.94%) as a yellow solid. LCMS: C₂₈H₂₈ClF₂N₇O₂S requires: 599.2, found: m/z=600.2 [M+H]⁺. ¹H NMR (300 MHz, Methanol-d₄) δ 8.80-8.70 (m, 2H), 8.66 (s, 1H), 8.13-8.04 (m, 2H), 7.88-7.73 (m, 2H), 7.68-7.61 (m, 1H), 7.41-7.24 (m, 2H), 3.53-3.39 (m, 8H), 3.36-3.34 (m, 2H), 3.32-3.29 (m, 2H), 2.02-1.63 (m, 4H).

### Synthesis of N-(5-chloro-3-{1-[2,6-difluoro-4-(piperazin-1-yl)phenyl]-3-(pyridin-4-yl)pyrazol-4-yl}-2-fluorophenyl)pyrrolidine-1-sulfonamide Trifluoroacetic Acid Salt (7)

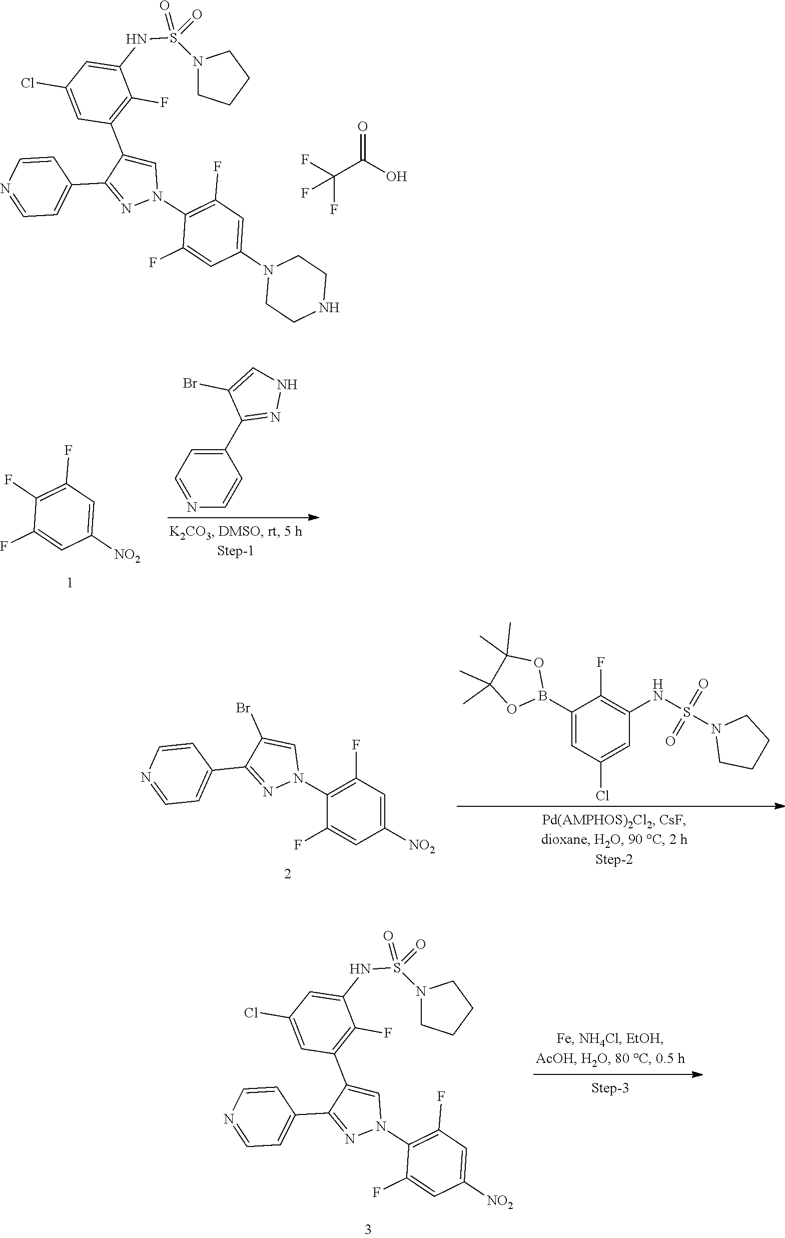

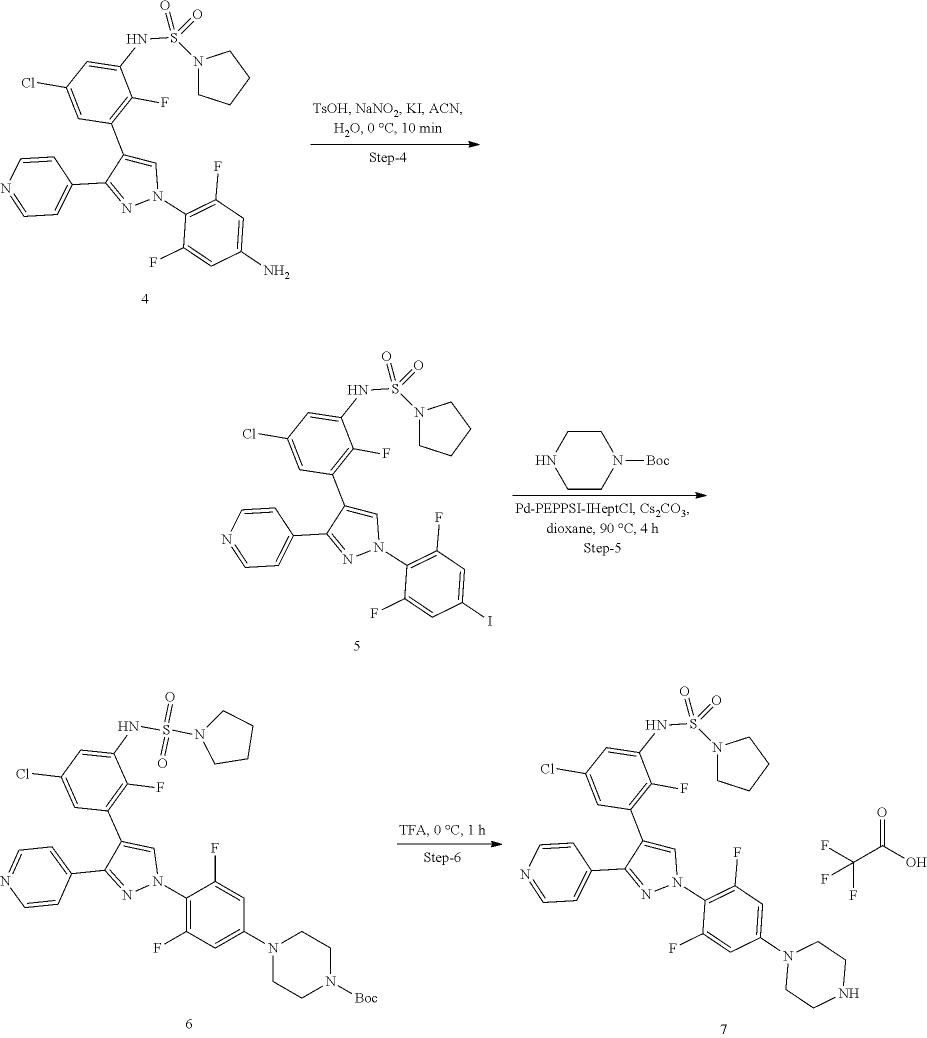 [SMILES: Nc1cc(F)c(-n2cc(-c3cc(Cl)cc(NS(=O)(=O)N4CCCC4)c3F)c(-c3ccncc3)n2)c(F)c1 ; O=S(=O)(Nc1cc(Cl)cc(-c2cn(-c3c(F)cc(I)cc3F)nc2-c2ccncc2)c1F)N1CCCC1 ; CC(C)(C)OC(=O)N1CCNCC1 ; CC(C)(C)OC(=O)N1CCN(c2cc(F)c(-n3cc(-c4cc(Cl)cc(NS(=O)(=O)N5CCCC5)c4F)c(-c4ccncc4)n3)c(F)c2)CC1 ; O=S(=O)(Nc1cc(Cl)cc(-c2cn(-c3c(F)cc(N4CCNCC4)cc3F)nc2-c2ccncc2)c1F)N1CCCC1 ; O=C(O)C(F)(F)F]

### Step-1: Synthesis of 4-[4-bromo-1-(2,6-difluoro-4-nitrophenyl)pyrazol-3-yl]pyridine (2)

To a mixture of 4-(4-bromo-1H-pyrazol-3-yl)pyridine (5.8 g, 25.886 mmol, 1 equiv) in DMSO (50 mL) was added K₂CO₃ (5.4 g, 38.829 mmol, 1.5 equiv) and 1,2,3-trifluoro-5-nitrobenzene (5.5 g, 31.063 mmol, 1.2 equiv). The resulting mixture was stirred at room temperature for 5 h. The resulting mixture was then extracted with CH₂Cl₂. The combined organic layers were washed with water and dried over anhydrous Na₂SO₄. After filtration, the filtrate was concentrated under reduced pressure. The residue was purified by silica gel column chromatography eluted with PE:EtOAc (3:1) to afford 4-[4-bromo-1-(2,6-difluoro-4-nitrophenyl)pyrazol-3-yl]pyridine (9 g, 82.10%) as a white solid. LCMS: C₁₄H₇BrF₂N₄O₂ requires: 379.9, found: m/z=380.9 [M+H]⁺.

### Step-2: Synthesis of N-{5-chloro-3-[1-(2,6-difluoro-4-nitrophenyl)-3-(pyridin-4-yl)pyrazol-4-yl]-2-fluorophenyl}pyrrolidine-1-sulfonamide (3)

To a mixture of 4-[4-bromo-1-(2,6-difluoro-4-nitrophenyl)pyrazol-3-yl]pyridine (8.8 g, 23.089 mmol, 1 equiv) and N-[5-chloro-2-fluoro-3-(4,4,5,5-tetramethyl-1,3,2-dioxaborolan-2-yl)phenyl]pyrrolidine-1-sulfonamide (9.3 g, 23.089 mmol, 1 equiv) in dioxane (100 mL) was added Pd(AMPHOS)₂Cl₂ (1.6 g, 2.309 mmol, 0.1 equiv) and CsF (7.0 g, 46.178 mmol, 2 equiv) in H₂O (20 mL). The resulting mixture was stirred at 90° C. for 2 h under a nitrogen atmosphere. The resulting mixture was then concentrated under vacuum. The residue was purified by silica gel column chromatography eluted with PE:EtOAc (1:1) to afford N-{5-chloro-3-[1-(2,6-difluoro-4-nitrophenyl)-3-(pyridin-4-yl)pyrazol-4-yl]-2-fluorophenyl}pyrrolidine-1-sulfonamide (5.65 g, 33.81%) as a yellow solid. LCMS: C₂₄H₁₈ClF₃N₆O₄S requires: 578.1, found: m/z=579.1 [M+H]⁺.

### Step-3: Synthesis of N-{3-[1-(4-amino-2,6-difluorophenyl)-3-(pyridin-4-yl)pyrazol-4-yl]-5-chloro-2-fluorophenyl}pyrrolidine-1-sulfonamide (4)

A mixture of Fe (2.7 g, 48.365 mmol, 5 equiv) and NH₄Cl (50 mg, 0.967 mmol, 0.1 equiv) in AcOH (2 mL) and H₂O (10 mL) was stirred at 80° C. for 5 min. To the above mixture was added N-{5-chloro-3-[1-(2,6-difluoro-4-nitrophenyl)-3-(pyridin-4-yl)pyrazol-4-yl]-2-fluorophenyl}pyrrolidine-1-sulfonamide (5.6 g, 9.673 mmol, 1 equiv) in EtOH (100 mL). The resulting mixture was stirred at 80° C. for 0.5 h. The resulting mixture was then concentrated under vacuum. The residue was purified by silica gel column chromatography eluted with PE:EtOAc (1:4) to afford N-{3-[1-(4-amino-2,6-difluorophenyl)-3-(pyridin-4-yl)pyrazol-4-yl]-5-chloro-2-fluorophenyl}pyrrolidine-1-sulfonamide (4.33 g, 65.24%) as a yellow solid. LCMS: C₂₄H₂₀ClF₃N₆O₂S requires: 548.1, found: m/z=549.2 [M+H]⁺.

### Step-4: Synthesis of N-{5-chloro-3-[1-(2,6-difluoro-4-iodophenyl)-3-(pyridin-4-yl)pyrazol-4-yl]-2-fluorophenyl}pyrrolidine-1-sulfonamide (5)

To a mixture of N-{3-[1-(4-amino-2,6-difluorophenyl)-3-(pyridin-4-yl)pyrazol-4-yl]-5-chloro-2-fluorophenyl}pyrrolidine-1-sulfonamide (3.7 g, 6.685 mmol, 1 equiv) and TsOH (3.5 g, 20.055 mmol, 3 equiv) in ACN (30 mL) was added NaNO₂ (1.4 g, 20.055 mmol, 3 equiv) in H₂O (3 mL) and KI (2.8 g, 16.712 mmol, 2.5 equiv) at 0° C. The resulting mixture was stirred at 0° C. for 10 min and then was warmed to room temperature. The mixture was stirred at room temperature for an additional 30 min. The mixture was then basified to pH=8 with saturated aqueous Na₂CO₃. The resulting mixture was extracted with EtOAc. The combined organic layers were washed with water and dried over anhydrous Na₂SO₄. After filtration, the filtrate was concentrated under reduced pressure. The residue was purified by silica gel column chromatography eluted with PE:EtOAc (3:2) to afford N-{5-chloro-3-[1-(2,6-difluoro-4-iodophenyl)-3-(pyridin-4-yl)pyrazol-4-yl]-2-fluorophenyl}pyrrolidine-1-sulfonamide (850 mg, 15.42%) as a brown solid. LCMS: C₂₄H₁₈ClF₃IN₅O₂S requires: 658.9, found: m/z=659.9 [M+H]⁺.

### Step-5: Synthesis of tert-butyl 4-[4-(4-{5-chloro-2-fluoro-3-[(pyrrolidine-1-sulfonyl)amino]phenyl}-3-(pyridin-4-yl)pyrazol-1-yl)-3,5-difluorophenyl]piperazine-1-carboxylate (6)

To a mixture of N-{5-chloro-3-[1-(2,6-difluoro-4-iodophenyl)-3-(pyridin-4-yl)pyrazol-4-yl]-2-fluorophenyl}pyrrolidine-1-sulfonamide (700 mg, 1.061 mmol, 1 equiv) and tert-butyl piperazine-1-carboxylate (494 mg, 2.652 mmol, 2.5 equiv) in dioxane (10 mL) was added Pd-PEPPSI-IHeptCl (103 mg, 0.106 mmol, 0.1 equiv) and Cs₂CO₃ (864 mg, 2.652 mmol, 2.5 equiv). The resulting mixture was stirred at 90° C. for 4 h under a nitrogen atmosphere. The resulting mixture was then concentrated under vacuum. The residue was purified by silica gel column chromatography eluted with PE:EtOAc (1:2) to afford tert-butyl 4-[4-(4-{5-chloro-2-fluoro-3-[(pyrrolidine-1-sulfonyl)amino]phenyl}-3-(pyridin-4-yl)pyrazol-1-yl)-3,5-difluorophenyl]piperazine-1-carboxylate (430 mg, 44.02%) as a brown solid. LCMS: C₃₃H₃₅ClF₃N₇O₄S requires: 717.2, found: m/z=718.4 [M+H]⁺.

### Step-6: Synthesis of N-(5-chloro-3-{1-[2,6-difluoro-4-(piperazin-1-yl)phenyl]-3-(pyridin-4-yl)pyrazol-4-yl}-2-fluorophenyl)pyrrolidine-1-sulfonamide Trifluoroacetic Acid Salt (7)

A mixture of tert-butyl 4-[4-(4-{5-chloro-2-fluoro-3-[(pyrrolidine-1-sulfonyl)amino]phenyl}-3-(pyridin-4-yl)pyrazol-1-yl)-3,5-difluorophenyl]piperazine-1-carboxylate (430 mg, 0.599 mmol, 1 equiv) in TFA (5 mL) was stirred at 0° C. for one hour. The resulting mixture was then concentrated under vacuum. The crude product was purified by prep-HPLC with the following conditions: Column: XSelect CSH Prep C18 OBD Column, 30×150 mm, 5 μm; Mobile Phase A: Water (0.05% TFA), Mobile Phase B: ACN; Flow rate: 60 mL/min; Gradient: 5% B to 50% B in 12 min; Wave Length: 254/220 nm to afford N-(5-chloro-3-{1-[2,6-difluoro-4-(piperazin-1-yl)phenyl]-3-(pyridin-4-yl)pyrazol-4-yl}-2-fluorophenyl)pyrrolidine-1-sulfonamide trifluoroacetic acid salt (163.9 mg, 37.09%) as a light yellow solid. LCMS: C₂₈H₂₇ClF₃ requires: 617.2, found: m/z=618.2 [M+H]⁺. ¹H NMR (400 MHz, Methanol-d₄) δ 8.75-8.69 (m, 2H), 8.28-8.24 (m, 1H), 8.00-7.94 (m, 2H), 7.67-7.60 (m, 1H), 7.33-7.27 (m, 1H), 6.99-6.90 (m, 2H), 3.68-3.61 (m, 4H), 3.45-3.38 (m, 4H), 3.34-3.28 (m, 4H), 1.96-1.85 (m, 4H).

### Synthesis of N-(5-chloro-3-{1-[2,5-difluoro-4-(piperazin-1-yl)phenyl]-3-(pyridin-4-yl)pyrazol-4-yl}-2-fluorophenyl)pyrrolidine-1-sulfonamide Trifluoroacetic Acid Salt (5)

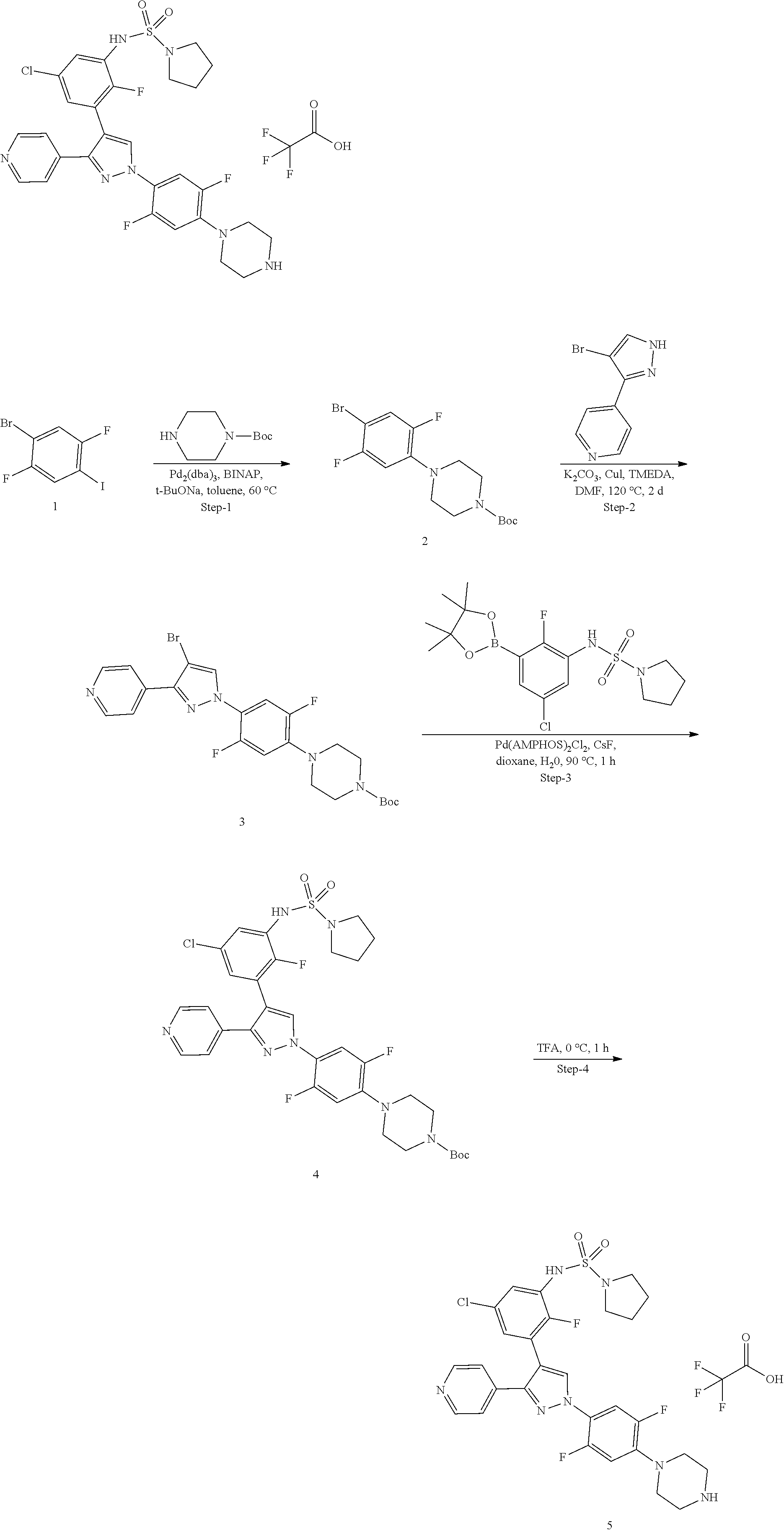

### Step-1: Synthesis of tert-butyl 4-(4-bromo-2,5-difluorophenyl)piperazine-1-carboxylate (2)

To a mixture of 1-bromo-2,5-difluoro-4-iodobenzene (14.5 g, 45.471 mmol, 1 equiv) and tert-butyl piperazine-1-carboxylate (10.6 g, 56.839 mmol, 1.25 equiv) in toluene (150 mL) was added t-BuONa (8.7 g, 90.942 mmol, 2 equiv), BINAP (5.7 g, 9.094 mmol, 0.2 equiv), and Pd₂(dba)₃ (4.2 g, 4.547 mmol, 0.1 equiv). The resulting mixture was stirred at 60° C. overnight under a nitrogen atmosphere. The resulting mixture was then concentrated under vacuum. The residue was purified by silica gel column chromatography eluted with PE:EtOAc (4:1) to afford tert-butyl 4-(4-bromo-2,5-difluorophenyl)piperazine-1-carboxylate (11 g, 51.30%) as a light yellow solid. LCMS: C₁₅H₁₉BrF₂N₂O₂ requires: 376.0, found: m/z=377.1 [M+H]⁺.

### Step-2: Synthesis of tert-butyl 4-{4-[4-bromo-3-(pyridin-4-yl)pyrazol-1-yl]-2,5-difluorophenyl}piperazine-1-carboxylate (3)

To a mixture of tert-butyl 4-(4-bromo-2,5-difluorophenyl)piperazine-1-carboxylate (11 g, 29.160 mmol, 1 equiv) and 4-(4-bromo-1H-pyrazol-3-yl)pyridine (7.2 g, 32.076 mmol, 1.1 equiv) in DMF (150 mL) was added K₂CO₃ (12.1 g, 87.480 mmol, 3 equiv), CuI (1.1 g, 5.832 mmol, 0.2 equiv), and TMEDA (1.4 g, 11.664 mmol, 0.4 equiv). The resulting mixture was stirred at 120° C. for two days under a nitrogen atmosphere. The resulting mixture was then extracted with EtOAc. The combined organic layers were washed with water and dried over anhydrous Na₂SO₄. After filtration, the filtrate was concentrated under reduced pressure. The residue was purified by silica gel column chromatography eluted with PE:EtOAc (1:1). The residue was purified by trituration with EtOAc. This resulted in tert-butyl 4-{4-[4-bromo-3-(pyridin-4-yl)pyrazol-1-yl]-2,5-difluorophenyl}piperazine-1-carboxylate (900 mg, 5.34%) as a brown solid. LCMS: C₂₃H₂₄BrF₂N₅O₂ requires: 519.1, found: m/z=520.3 [M+H]⁺.

### Step-3: Synthesis of tert-butyl 4-[4-(4-{5-chloro-2-fluoro-3-[(pyrrolidine-1-sulfonyl)amino]phenyl}-3-(pyridin-4-yl)pyrazol-1-yl)-2,5-difluorophenyl]piperazine-1-carboxylate (4)

To a mixture of tert-butyl 4-{4-[4-bromo-3-(pyridin-4-yl)pyrazol-1-yl]-2,5-difluorophenyl}piperazine-1-carboxylate (900 mg, 1.730 mmol, 1 equiv) and N-[5-chloro-2-fluoro-3-(4,4,5,5-tetramethyl-1,3,2-dioxaborolan-2-yl)phenyl]pyrrolidine-1-sulfonamide (700 mg, 1.730 mmol, 1 equiv) in dioxane (10 mL) was added Pd(AMPHOS)₂Cl₂ (245 mg, 0.346 mmol, 0.2 equiv) and CsF (525 mg, 3.460 mmol, 2 equiv) in H₂O (2 mL). The resulting mixture was stirred at 90° C. for 1 h under a nitrogen atmosphere. The resulting mixture was then concentrated under vacuum. The residue was purified by silica gel column chromatography eluted with PE:EtOAc (2:3). The residue was then purified by trituration with EtOAc. This resulted in tert-butyl 4-[4-(4-{5-chloro-2-fluoro-3-[(pyrrolidine-1-sulfonyl)amino]phenyl}-3-(pyridin-4-yl)pyrazol-1-yl)-2,5-difluorophenyl]piperazine-1-carboxylate (550 mg, 42.07%) as a white solid. LCMS: C₃₃H₃₅ClF₃N₇O₄S requires: 717.2, found: m/z=718.3 [M+H]⁺.

### Step-4: Synthesis of N-(5-chloro-3-{1-[2,5-difluoro-4-(piperazin-1-yl)phenyl]-3-(pyridin-4-yl)pyrazol-4-yl}-2-fluorophenyl)pyrrolidine-1-sulfonamide Trifluoroacetic Acid Salt (5)

A mixture of tert-butyl 4-[4-(4-{5-chloro-2-fluoro-3-[(pyrrolidine-1-sulfonyl)amino]phenyl}-3-(pyridin-4-yl)pyrazol-1-yl)-2,5-difluorophenyl]piperazine-1-carboxylate (550 mg, 0.766 mmol, 1 equiv) in TFA (6 mL) was stirred at 0° C. for one hour. The resulting mixture was then concentrated under vacuum and lyophilized. This resulted in N-(5-chloro-3-f{1-[2,5-difluoro-4-(piperazin-1-yl)phenyl]-3-(pyridin-4-yl)pyrazol-4-yl}-2-fluorophenyl)pyrrolidine-1-sulfonamide trifluoroacetic acid salt (345.2 mg, crude) as a yellow solid. LCMS: C₂₈H₂₇ClF₃N₇O₂S requires: 617.1, found: m/z=618.2 [M+H]⁺. ¹H NMR (400 MHz, Methanol-d₄) δ 8.82-8.75 (m, 2H), 8.51-8.46 (m, 1H), 8.17-8.10 (m, 2H), 7.90-7.81 (m, 1H), 7.68-7.61 (m, 1H), 7.37-7.31 (m, 1H), 7.29-7.18 (m, 1H), 3.50-3.44 (m, 8H), 3.38-3.29 (m, 4H), 1.97-1.86 (m, 4H).

### Synthesis of N-(5-chloro-2-fluoro-3-{1-[5-(piperazin-1-yl)pyrimidin-2-yl]-3-(pyridin-4-yl)pyrazol-4-yl}phenyl)pyrrolidine-1-sulfonamide Trifluoroacetic Acid Salt

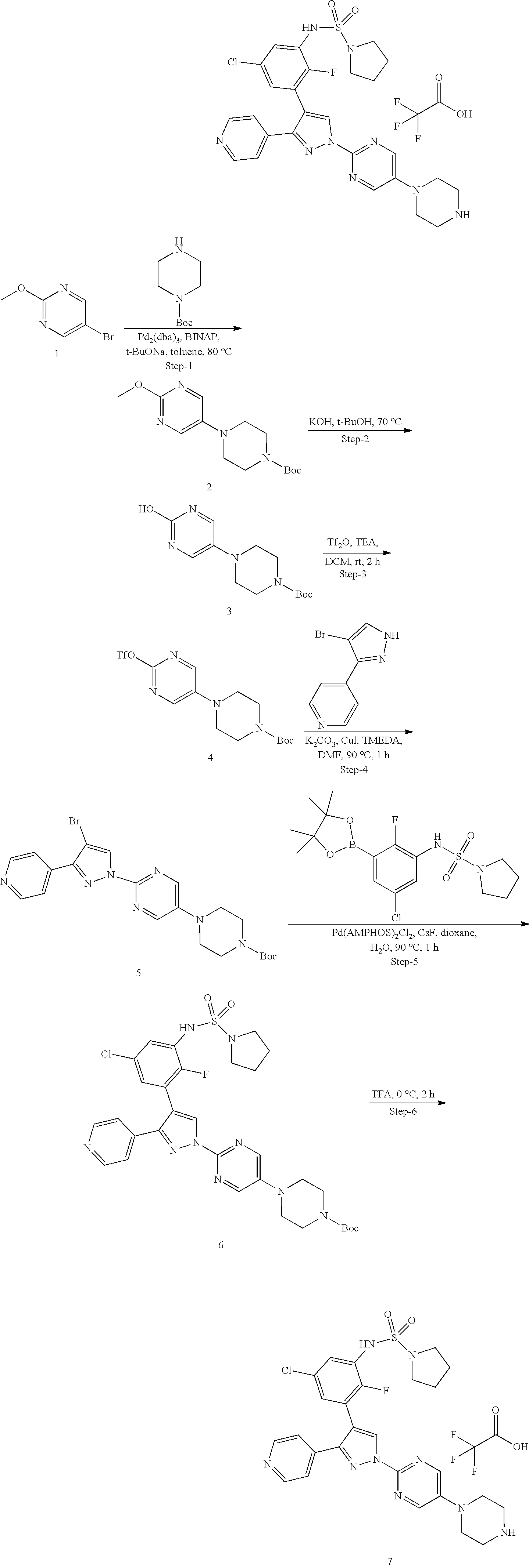

### Step-1: Synthesis of tert-butyl 4-(2-methoxypyrimidin-5-yl)piperazine-1-carboxylate (2)

To a mixture of 5-bromo-2-methoxypyrimidine (7.5 g, 39.680 mmol, 1 equiv) and tert-butyl piperazine-1-carboxylate (7.39 g, 39.680 mmol, 1 equiv) in toluene (30 mL) was added BINAP (0.49 g, 0.794 mmol, 0.02 equiv), t-BuONa (7.63 g, 79.360 mmol, 2 equiv), and Pd₂(dba)₃ (0.47 g, 0.518 mmol, 0.01 equiv). The resulting mixture was stirred at 80° C. overnight under a nitrogen atmosphere. The resulting mixture was then concentrated under vacuum. The residue was purified by silica gel column chromatography eluted with PE:EtOAc (1:1) to afford tert-butyl 4-(2-methoxypyrimidin-5-yl)piperazine-1-carboxylate (4.3 g, 25.36%) as a yellow solid. LCMS: C₁₄H₂₂N₄O₃ requires: 294.2, found: m/z=295.2 [M+H]⁺.

### Step-2: Synthesis of tert-butyl 4-(2-hydroxypyrimidin-5-yl)piperazine-1-carboxylate (3)

To a mixture of tert-butyl 4-(2-methoxypyrimidin-5-yl)piperazine-1-carboxylate (3.21 g, 10.905 mmol, 1 equiv) in t-BuOH (30 mL) was added KOH (0.86 g, 15.267 mmol, 1.4 equiv). The resulting mixture was stirred at 70° C. overnight. The mixture was then neutralized to pH=7 with aqueous HCl (2 M). The resulting mixture was concentrated under vacuum. The residue was purified by silica gel column chromatography eluted with CH₂Cl₂:MeOH (10:1) to afford tert-butyl 4-(2-hydroxypyrimidin-5-yl)piperazine-1-carboxylate (2.61 g, 85.38%) as a yellow solid. LCMS: C₁₃H₂₀N₄O₃ requires: 280.2, found: m/z=281.2 [M+H]⁺.

### Step-3: Synthesis of tert-butyl 4-[2-(trifluoromethanesulfonyloxy)pyrimidin-5-yl]piperazine-1-carboxylate (4)

To a mixture of tert-butyl 4-(2-hydroxypyrimidin-5-yl)piperazine-1-carboxylate (2.41 g, 8.597 mmol, 1 equiv) in DCM (7 mL) was added TEA (2.61 g, 25.791 mmol, 3 equiv) and Tf₂O (4.85 g, 17.194 mmol, 2 equiv) at 0° C. The resulting mixture was then warmed and stirred at room temperature for 2 h under a nitrogen atmosphere. The resulting mixture was then concentrated under vacuum. The residue was purified by silica gel column chromatography eluted with PE:EtOAc (3:1) to afford tert-butyl 4-[2-(trifluoromethanesulfonyloxy)pyrimidin-5-yl]piperazine-1-carboxylate (1.4159 g, 37.94%) as a light brown solid. LCMS: C₁₄H₁₉F₃N₄O₅S requires: 412.1, found: m/z=413.1 [M+H]⁺.

### Step-4: Synthesis of tert-butyl 4-{2-[4-bromo-3-(pyridin-4-yl)pyrazol-1-yl]pyrimidin-5-yl}piperazine-1-carboxylate (5)

To a mixture of tert-butyl 4-[2-(trifluoromethanesulfonyloxy)pyrimidin-5-yl]piperazine-1-carboxylate (3 g, 7.275 mmol, 1 equiv) and 4-(4-bromo-1H-pyrazol-3-yl)pyridine (2.5 g, 10.913 mmol, 1.5 equiv) in DMF (40 mL) was added K₂CO₃ (3.0 g, 21.825 mmol, 3 equiv), CuI (277 mg, 1.455 mmol, 0.2 equiv), and TMEDA (338 mg, 2.910 mmol, 0.4 equiv). The resulting mixture was stirred at 90° C. for one hour under a nitrogen atmosphere. The resulting mixture was then extracted with EtOAc. The combined organic layers were washed with water and dried over anhydrous Na₂SO₄. After filtration, the filtrate was concentrated under reduced pressure. The residue was purified by silica gel column chromatography eluted with EtOAc:MeOH (19:1). The residue was then purified by trituration with EtOAc. This resulted in tert-butyl 4-{2-[4-bromo-3-(pyridin-4-yl)pyrazol-1-yl]pyrimidin-5-yl}piperazine-1-carboxylate (900 mg, 22.89%) as a brown solid. LCMS: C₂₁H₂₄BrN₇O₂ requires: 485.1, found: m/z=486.3 [M+H]⁺.

### Step-5: Synthesis of tert-butyl 4-[2-(4-{5-chloro-2-fluoro-3-[(pyrrolidine-1-sulfonyl)amino]phenyl}-3-(pyridin-4-yl)pyrazol-1-yl)pyrimidin-5-yl]piperazine-1-carboxylate (6)

To a mixture of tert-butyl 4-{2-[4-bromo-3-(pyridin-4-yl)pyrazol-1-yl]pyrimidin-5-yl}piperazine-1-carboxylate (900 mg, 1.850 mmol, 1 equiv) and N-[5-chloro-2-fluoro-3-(4,4,5,5-tetramethyl-1,3,2-dioxaborolan-2-yl)phenyl]pyrrolidine-1-sulfonamide (749 mg, 1.850 mmol, 1 equiv) in dioxane (10 mL) was added Pd(AMPHOS)₂Cl₂ (131 mg, 0.185 mmol, 0.1 equiv) and CsF (562 mg, 3.700 mmol, 2 equiv) in H₂O (2 mL). The resulting mixture was stirred at 90° C. for one hour under a nitrogen atmosphere. The resulting mixture was concentrated under vacuum. The residue was purified by silica gel column chromatography eluted with EtOAc:MeOH (9:1). The residue was then purified by trituration with EtOAc. This resulted in tert-butyl 4-[2-(4-{5-chloro-2-fluoro-3-[(pyrrolidine-1-sulfonyl)amino]phenyl}-3-(pyridin-4-yl)pyrazol-1-yl)pyrimidin-5-yl]piperazine-1-carboxylate (700 mg, 44.23%) as a brown semi-solid. LCMS: C₃₁H₃₅ClFN₉O₄S requires: 683.2, found: m/z=684.3 [M+H]⁺.

### Step-6: Synthesis of N-(5-chloro-2-fluoro-3-{1-[5-(piperazin-1-yl)pyrimidin-2-yl]-3-(pyridin-4-yl)pyrazol-4-yl}phenyl)pyrrolidine-1-sulfonamide Trifluoroacetic Acid Salt (7)

A mixture of tert-butyl 4-[2-(4-{5-chloro-2-fluoro-3-[(pyrrolidine-1-sulfonyl)amino]phenyl}-3-(pyridin-4-yl)pyrazol-1-yl)pyrimidin-5-yl]piperazine-1-carboxylate (700 mg, 1.023 mmol, 1 equiv) in TFA (7 mL) was stirred at 0° C. for 2 h. The resulting mixture was then concentrated under vacuum. The crude product was purified by prep-HPLC with the following conditions: Column: XSelect CSH Prep C18 OBD Column, 19×250 mm, 5 μm; Mobile Phase A: Water (0.05% TFA), Mobile Phase B: MeOH; Flow rate: 25 mL/min; Gradient: 33% B to 48% B in 10 min; Wave Length: 254/220 nm to afford N-(5-chloro-2-fluoro-3-{1-[5-(piperazin-1-yl)pyrimidin-2-yl]-3-(pyridin-4-yl)pyrazol-4-yl}phenyl)pyrrolidine-1-sulfonamide trifluoroacetic acid salt (342.5 mg, 46.67%) as a yellow solid. LCMS: C₂₆H₂₇ClFN₉O₂S requires: 583.2, found: m/z=584.1 [M+H]⁺. ¹H NMR (300 MHz, Methanol-d₄) δ 8.98-8.91 (m, 1H), 8.84-8.76 (m, 2H), 8.75-8.65 (m, 2H), 8.22-8.14 (m, 2H), 7.69-7.60 (m, 1H), 7.40-7.30 (m, 1H), 3.73-3.64 (m, 4H), 3.53-3.43 (m, 4H), 3.38-3.28 (m, 4H), 1.97-1.86 (m, 4H).

### Synthesis of N-[5-(difluoromethyl)-2-fluoro-3-{1-[4-(piperazin-1-yl)phenyl]-3-(pyridin-4-yl)pyrazol-4-yl}phenyl]pyrrolidine-1-sulfonamide Trifluoroacetic Acid Salt (3)

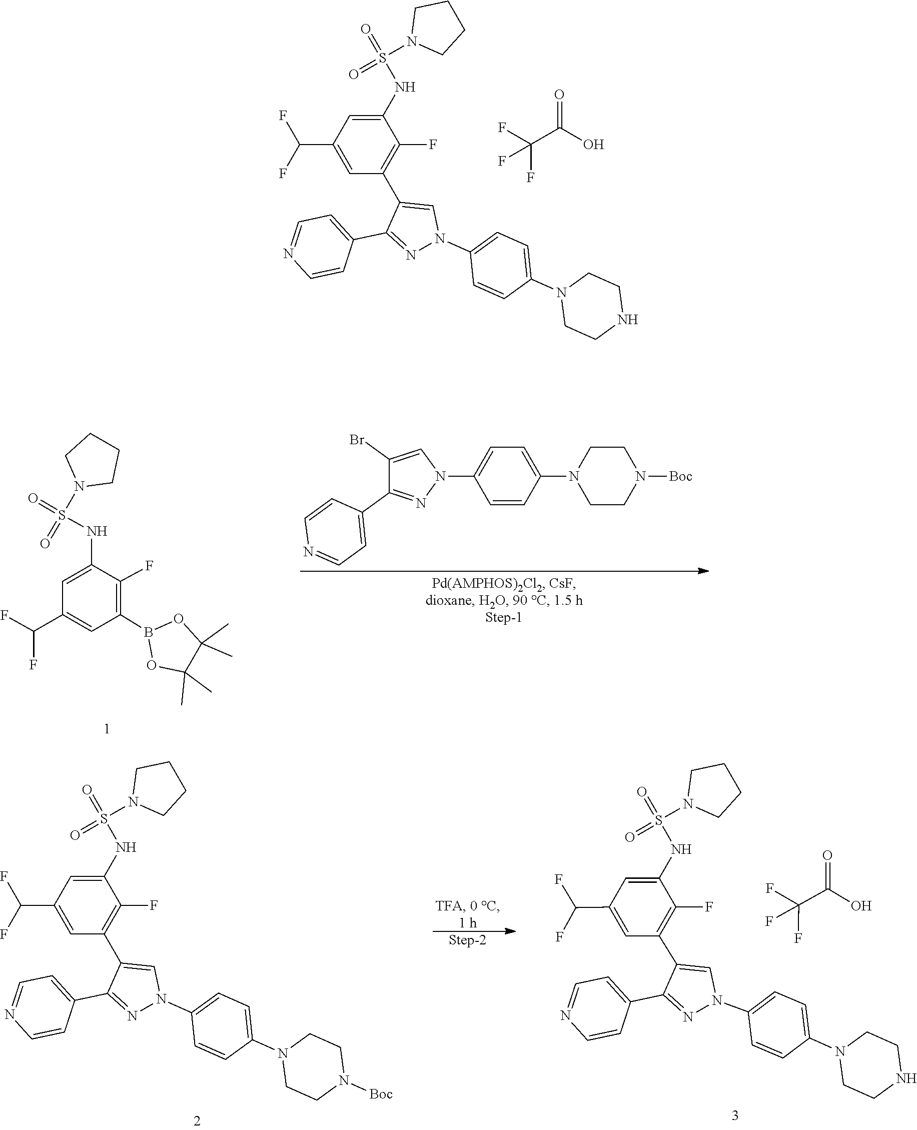

### Step-1: Synthesis of tert-butyl 4-(4-{4-[5-(difluoromethyl)-2-fluoro-3-[(pyrrolidine-1-sulfonyl)amino]phenyl]-3-(pyridin-4-yl)pyrazol-1-yl}phenyl)piperazine-1-carboxylate (2)

To a mixture of tert-butyl 4-{4-[4-bromo-3-(pyridin-4-yl)pyrazol-1-yl]phenyl}piperazine-1-carboxylate (1.2 g, 2.477 mmol, 1 equiv) and N-[5-(difluoromethyl)-2-fluoro-3-(4,4,5,5-tetramethyl-1,3,2-dioxaborolan-2-yl)phenyl]pyrrolidine-1-sulfonamide (1.04 g, 2.477 mmol, 1 equiv) in dioxane (40 mL) was added Pd(AMPHOS)₂Cl₂ (175 mg, 0.248 mmol, 0.1 equiv) and CsF (753 mg, 4.954 mmol, 2 equiv) in H₂O (8 mL). The resulting mixture was stirred at 90° C. for 1.5 h under a nitrogen atmosphere. The resulting mixture was then concentrated under reduced pressure. The residue was purified by silica gel column chromatography eluted with DCM:EtOAc (1:1) to afford tert-butyl 4-(4-{4-[5-(difluoromethyl)-2-fluoro-3-[(pyrrolidine-1-sulfonyl)amino]phenyl]-3-(pyridin-4-yl)pyrazol-1-yl}phenyl)piperazine-1-carboxylate (450 mg, 20.83%) as an off-white solid. LCMS: C₃₄H₃₈F₃N₇O₄S requires: 697.3, found: m/z=698.3 [M+H]⁺.

### Step-2: Synthesis of N-[5-(difluoromethyl)-2-fluoro-3-{1-[4-(piperazin-1-yl)phenyl]-3-(pyridin-4-yl)pyrazol-4-yl}phenyl]pyrrolidine-1-sulfonamide Trifluoroacetic Acid Salt (3)

A mixture of tert-butyl 4-(4-{4-[5-(difluoromethyl)-2-fluoro-3-[(pyrrolidine-1-sulfonyl)amino]phenyl]-3-(pyridin-4-yl)pyrazol-1-yl}phenyl)piperazine-1-carboxylate (440 mg, 0.631 mmol, 1 equiv) in TFA (3 mL) was stirred at 0° C. for one hour. The resulting mixture was then concentrated under reduced pressure. The crude product was purified by prep-HPLC with the following conditions: Column: XSelect CSH Prep C18 OBD Column, 30×150 mm, m; Mobile Phase A: Water (0.05% TFA), Mobile Phase B: ACN; Flow rate: 60 mL/min; Gradient: 5% B to 40% B in 12 min; Wave Length: 254/220 nm to afford N-[5-(difluoromethyl)-2-fluoro-3-{1-[4-(piperazin-1-yl)phenyl]-3-(pyridin-4-yl)pyrazol-4-yl}phenyl]pyrrolidine-1-sulfonamide trifluoroacetic acid salt (304.1 mg, 67.56%) as a yellow solid. LCMS: C₂₉H₃₀F₃N₇O₂S requires: 597.2, found: m/z=598.2 [M+H]⁺. ¹H NMR (400 MHz, Methanol-d₄) δ 8.83-8.70 (m, 2H), 8.62 (s, 1H), 8.21-8.07 (m, 2H), 7.98-7.87 (m, 2H), 7.86-7.76 (m, 1H), 7.60-7.49 (m, 1H), 7.35-7.19 (m, 2H), 7.06-6.66 (m, 1H), 3.60-3.50 (m, 4H), 3.49-3.40 (m, 4H), 3.36-3.28 (m, 4H), 2.02-1.82 (m, 4H).

### Synthesis of N-(5-chloro-3-{1-[4-(1,4-diazepan-1-yl)-2-fluorophenyl]-3-(pyridin-4-yl)pyrazol-4-yl}-2-fluorophenyl)pyrrolidine-1-sulfonamide Trifluoroacetic Acid Salt (6)

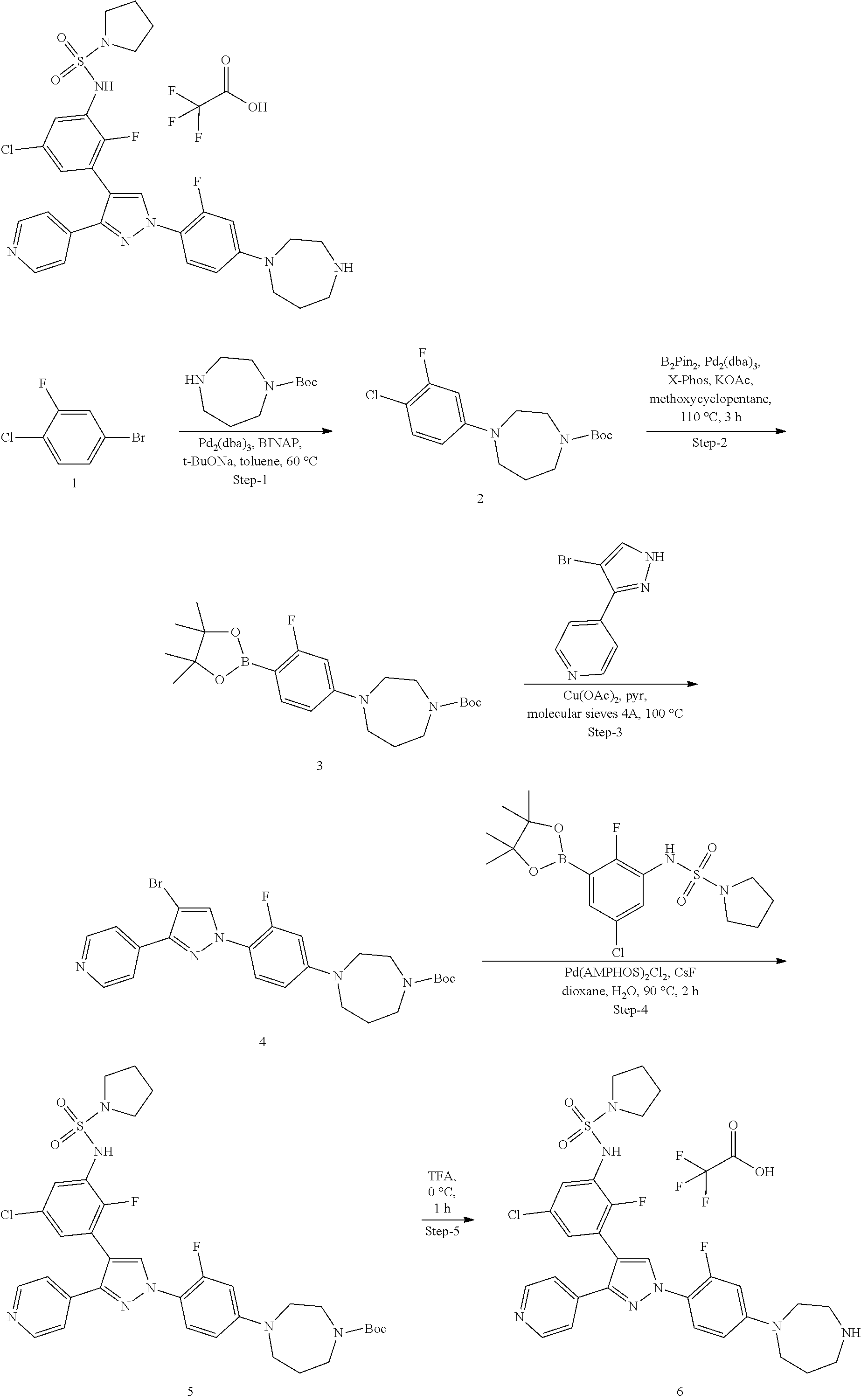

### Step-1: Synthesis of tert-butyl 4-(4-chloro-3-fluorophenyl)-1,4-diazepane-1-carboxylate (2)

To a mixture of 4-bromo-1-chloro-2-fluorobenzene (21 g, 100.267 mmol, 1 equiv) and tert-butyl 1,4-diazepane-1-carboxylate (25.10 g, 125.334 mmol, 1.25 equiv) in toluene (200 mL) was added BINAP (12.49 g, 20.053 mmol, 0.2 equiv), t-BuONa (19.27 g, 200.534 mmol, 2 equiv), and Pd₂(dba)₃ (9.18 g, 10.027 mmol, 0.1 equiv). The resulting mixture was stirred at 60° C. overnight under a nitrogen atmosphere. The resulting mixture was then concentrated under reduced pressure. The residue was purified by silica gel column chromatography eluted with PE:EtOAc (5:1) to afford tert-butyl 4-(4-chloro-3-fluorophenyl)-1,4-diazepane-1-carboxylate (30 g, 90.99%) as a brown oil. LCMS: C₁₆H₂₂ClFN₂O₂ requires: 328.1, found: m/z=329.3 [M+H]⁺.

### Step-2: Synthesis of tert-butyl 4-[3-fluoro-4-(4,4,5,5-tetramethyl-1,3,2-dioxaborolan-2-yl)phenyl]-1,4-diazepane-1-carboxylate (3)

To a mixture of tert-butyl 4-(4-chloro-3-fluorophenyl)-1,4-diazepane-1-carboxylate (27 g, 82.114 mmol, 1 equiv) and bis(pinacolato)diboron (B₂Pin₂) (31.28 g, 123.171 mmol, 1.5 equiv) in methoxycyclopentane (300 mL) was added AcOK (24.18 g, 246.342 mmol, 3 equiv), XPhos (2.35 g, 4.927 mmol, 0.06 equiv), and Pd₂(dba)₃ (2.26 g, 2.463 mmol, 0.03 equiv). The resulting mixture was stirred at 110° C. for 3 h under a nitrogen atmosphere. The resulting mixture was then concentrated under reduced pressure. The residue was purified by silica gel column chromatography eluted with PE:EtOAc (3:1) to afford tert-butyl 4-[3-fluoro-4-(4,4,5,5-tetramethyl-1,3,2-dioxaborolan-2-yl)phenyl]-1,4-diazepane-1-carboxylate (12 g, 34.77%) as a white solid. LCMS: C₂₂H₃₄BFN₂O₄ requires: 420.3, found: m/z=421.1 [M+H]⁺.

### Step-3: Synthesis of tert-butyl 4-{4-[4-bromo-3-(pyridin-4-yl)pyrazol-1-yl]-3-fluorophenyl}-1,4-diazepane-1-carboxylate (4)

To a mixture of tert-butyl 4-[3-fluoro-4-(4,4,5,5-tetramethyl-1,3,2-dioxaborolan-2-yl)phenyl]-1,4-diazepane-1-carboxylate (12 g, 28.549 mmol, 1 equiv) and 4-(4-bromo-1H-pyrazol-3-yl)pyridine (7.04 g, 31.404 mmol, 1.1 equiv) in pyridine (120 mL) was added Cu(OAc)₂ (10.37 g, 57.098 mmol, 2 equiv) and molecular sieves (4A) (12 g). The resulting mixture was stirred at 100° C. overnight under an oxygen atmosphere. The resulting mixture was then extracted with EtOAc. The combined organic layers were washed with water and dried over anhydrous Na₂SO₄. After filtration, the filtrate was concentrated under reduced pressure. The residue was purified by silica gel column chromatography eluted with PE:EtOAc (1:3) to afford tert-butyl 4-{4-[4-bromo-3-(pyridin-4-yl)pyrazol-1-yl]-3-fluorophenyl}-1,4-diazepane-1-carboxylate (3 g, 20.35%) as a white solid. LCMS: C₂₄H₂₇BrFN₅O₂ requires: 515.1, found: m/z=516.3 [M+H]⁺.

### Step-4: Synthesis of tert-butyl 4-[4-(4-{5-chloro-2-fluoro-3-[(pyrrolidine-1-sulfonyl)amino]phenyl}-3-(pyridin-4-yl)pyrazol-1-yl)-3-fluorophenyl]-1,4-diazepane-1-carboxylate (5)

To a mixture of tert-butyl 4-{4-[4-bromo-3-(pyridin-4-yl)pyrazol-1-yl]-3-fluorophenyl}-1,4-diazepane-1-carboxylate (1 g, 1.936 mmol, 1 equiv) and N-[5-chloro-2-fluoro-3-(4,4,5,5-tetramethyl-1,3,2-dioxaborolan-2-yl)phenyl]pyrrolidine-1-sulfonamide (0.78 g, 1.936 mmol, 1 equiv) in dioxane (10 mL) was added CsF (0.59 g, 3.872 mmol, 2 equiv) and Pd(AMPHOS)₂Cl₂ (0.14 g, 0.194 mmol, 0.1 equiv) in H₂O (2 mL). The resulting mixture was stirred at 90° C. for 2 h under a nitrogen atmosphere. The resulting mixture was then concentrated under reduced pressure. The residue was purified by silica gel column chromatography eluted with PE:EtOAc (1:4) to afford tert-butyl 4-[4-(4-{5-chloro-2-fluoro-3-[(pyrrolidine-1-sulfonyl)amino]phenyl}-3-(pyridin-4-yl)pyrazol-1-yl)-3-fluorophenyl]-1,4-diazepane-1-carboxylate (700 mg, 50.61%) as a grey solid. LCMS: C₃₄H₃₈ClF₂N₇O₄S requires: 713.2, found: m/z=714.3 [M+H]⁺.

### Step-5: Synthesis of N-(5-chloro-3-{1-[4-(1,4-diazepan-1-yl)-2-fluorophenyl}-3-(pyridin-4-yl)pyrazol-4-yl]-2-fluorophenyl)pyrrolidine-1-sulfonamide Trifluoroacetic Acid Salt (6)

A mixture of tert-butyl 4-[4-(4-{5-chloro-2-fluoro-3-[(pyrrolidine-1-sulfonyl)amino]phenyl}-3-(pyridin-4-yl)pyrazol-1-yl)-3-fluorophenyl]-1,4-diazepane-1-carboxylate (700 mg, 0.980 mmol, 1 equiv) in TFA (10 mL) was stirred at 0° C. for one hour. The resulting mixture was then concentrated under reduced pressure. The crude product was purified by prep-HPLC with the following conditions: Column: XSelect CSH Prep C18 OBD Column, 30×150 mm, 5 μm; Mobile Phase A: Water (0.05% TFA), Mobile Phase B: ACN; Flow rate: 60 mL/min; Gradient: 5% B to 40% B in 12 min; Wave Length: 254/220 nm to afford N-(5-chloro-3-f{1-[4-(1,4-diazepan-1-yl)-2-fluorophenyl]-3-(pyridin-4-yl)pyrazol-4-yl}-2-fluorophenyl)pyrrolidine-1-sulfonamide trifluoroacetic acid salt (347.2 mg, 48.65%) as a yellow solid. LCMS: C₂₉H₃₀ClF₂N₇O₂S requires: 613.2, found: m/z=614.3 [M+H]⁺. ¹H NMR (300 MHz, Methanol-d₄) δ 8.79-8.70 (m, 2H), 8.35-8.28 (m, 1H), 8.13-8.04 (m, 2H), 7.82-7.69 (m, 1H), 7.67-7.58 (m, 1H), 7.37-7.28 (m, 1H), 6.93-6.79 (m, 2H), 3.95-3.85 (m, 2H), 3.75-3.65 (m, 2H), 3.51-3.42 (m, 2H), 3.38-3.33 (m, 4H), 3.33-3.29 (m, 2H), 2.33-2.19 (m, 2H), 1.98-1.84 (m, 4H).

### Synthesis of N-(5-chloro-2-fluoro-3-{1-[6-(piperazin-1-yl)pyridin-3-yl]-3-(pyridin-4-yl)pyrazol-4-yl}phenyl)pyrrolidine-1-sulfonamide Trifluoroacetic Acid Salt (3)

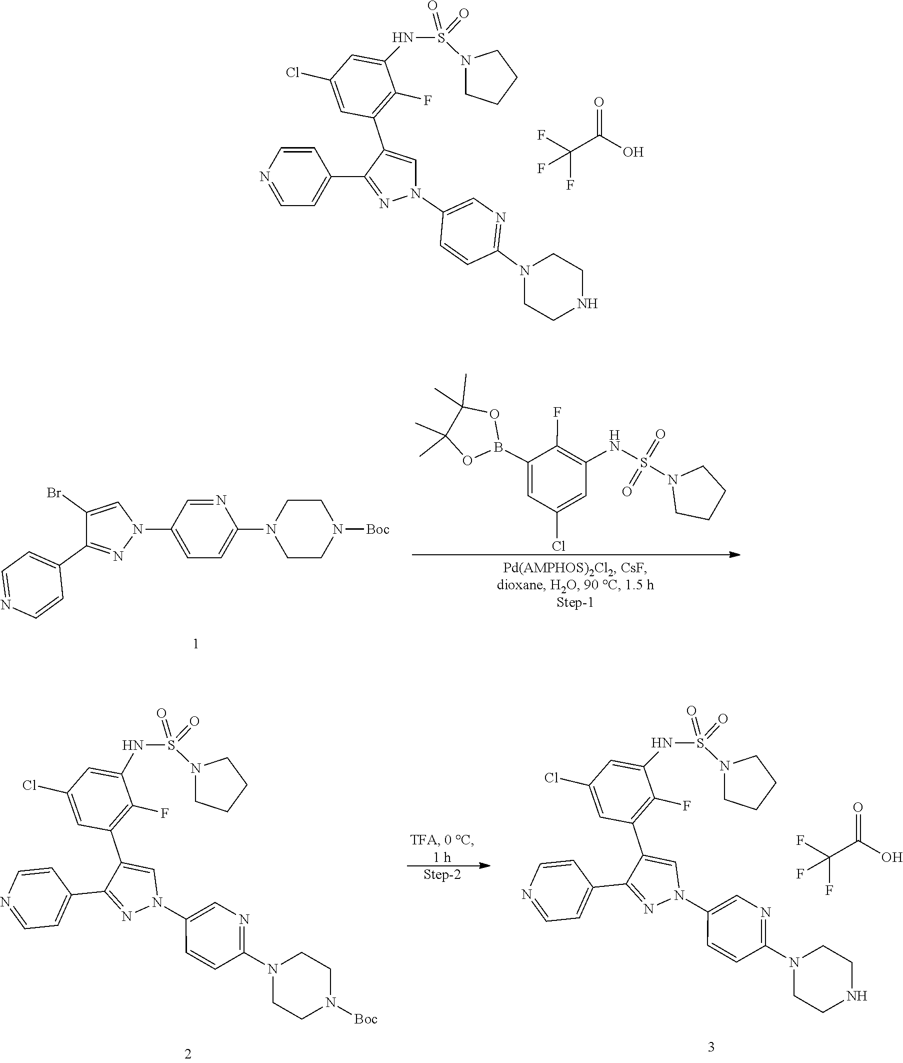

### Step-1: Synthesis of tert-butyl 4-[5-(4-{5-chloro-2-fluoro-3-[(pyrrolidine-1-sulfonyl)amino]phenyl}-3-(pyridin-4-yl)pyrazol-1-yl)pyridin-2-yl]piperazine-1-carboxylate (2)

To a mixture of tert-butyl 4-{5-[4-bromo-3-(pyridin-4-yl)pyrazol-1-yl]pyridin-2-yl}piperazine-1-carboxylate (1.2 g, 2.472 mmol, 1 equiv) and N-[5-chloro-2-fluoro-3-(4,4,5,5-tetramethyl-1,3,2-dioxaborolan-2-yl)phenyl]pyrrolidine-1-sulfonamide (1 g, 2.472 mmol, 1 equiv) in dioxane (40 mL) was added Pd(AMPHOS)₂Cl₂ (0.18 g, 0.247 mmol, 0.1 equiv) and CsF (0.75 g, 4.944 mmol, 2 equiv) in H₂O (8 mL). The resulting mixture was stirred at 90° C. for 1.5 h under a nitrogen atmosphere. The resulting mixture was then concentrated under reduced pressure. The residue was purified by silica gel column chromatography eluted with DCM:EtOAc (1:1) to afford tert-butyl 4-[5-(4-{5-chloro-2-fluoro-3-[(pyrrolidine-1-sulfonyl)amino]phenyl}-3-(pyridin-4-yl)pyrazol-1-yl)pyridin-2-yl]piperazine-1-carboxylate (700 mg, 33.15%) as an off-white solid. LCMS: C₃₂H₃₆ClFN₈O₄S requires: 682.2, found: m/z=683.2 [M+H]⁺.

### Step-2: Synthesis of N-(5-chloro-2-fluoro-3-{1-[6-(piperazin-1-yl)pyridin-3-yl]-3-(pyridin-4-yl)pyrazol-4-yl}phenyl)pyrrolidine-1-sulfonamide Trifluoroacetic Acid Salt (3)

A mixture of tert-butyl 4-[5-(4-{5-chloro-2-fluoro-3-[(pyrrolidine-1-sulfonyl)amino]phenyl}-3-(pyridin-4-yl)pyrazol-1-yl)pyridin-2-yl]piperazine-1-carboxylate (690 mg, 1.01 mmol, 1 equiv) in TFA (5 mL) was stirred at 0° C. for one hour. The resulting mixture was then concentrated under reduced pressure. The crude product was purified by prep-HPLC with the following conditions: Column: XSelect CSH Prep C18 OBD Column, 30×150 mm, 5 μm; Mobile Phase A: Water (0.05% TFA), Mobile Phase B: ACN; Flow rate: 60 mL/min; Gradient: 5% B to 40% B in 12 min; Wave Length: 254/220 nm to afford N-(5-chloro-2-fluoro-3-{1-[6-(piperazin-1-yl)pyridin-3-yl]-3-(pyridin-4-yl)pyrazol-4-yl}phenyl)pyrrolidine-1-sulfonamide trifluoroacetic acid salt (462.3 mg, 64.42%) as a yellow solid. LCMS: C₂₇H₂₈ClFN₈O₂S requires: 582.2, found: m/z=583.3 [M+H]⁺. ¹H NMR (400 MHz, Methanol-d₄) δ 8.86-8.72 (m, 3H), 8.61 (s, 1H), 8.24-8.18 (m, 1H), 8.17-8.11 (m, 2H), 7.70-7.61 (m, 1H), 7.42-7.33 (m, 1H), 7.13 (m, 1H), 4.05-3.86 (m, 4H), 3.42-3.36 (m, 4H), 3.35-3.30 (m, 4H), 2.00-1.84 (m, 4H).

### Synthesis of N-(2,5-difluoro-3-{1-[5-(piperazin-1-yl)pyrimidin-2-yl]-3-(pyridin-4-yl)pyrazol-4-yl}phenyl)pyrrolidine-1-sulfonamide Trifluoroacetic Acid Salt (3)

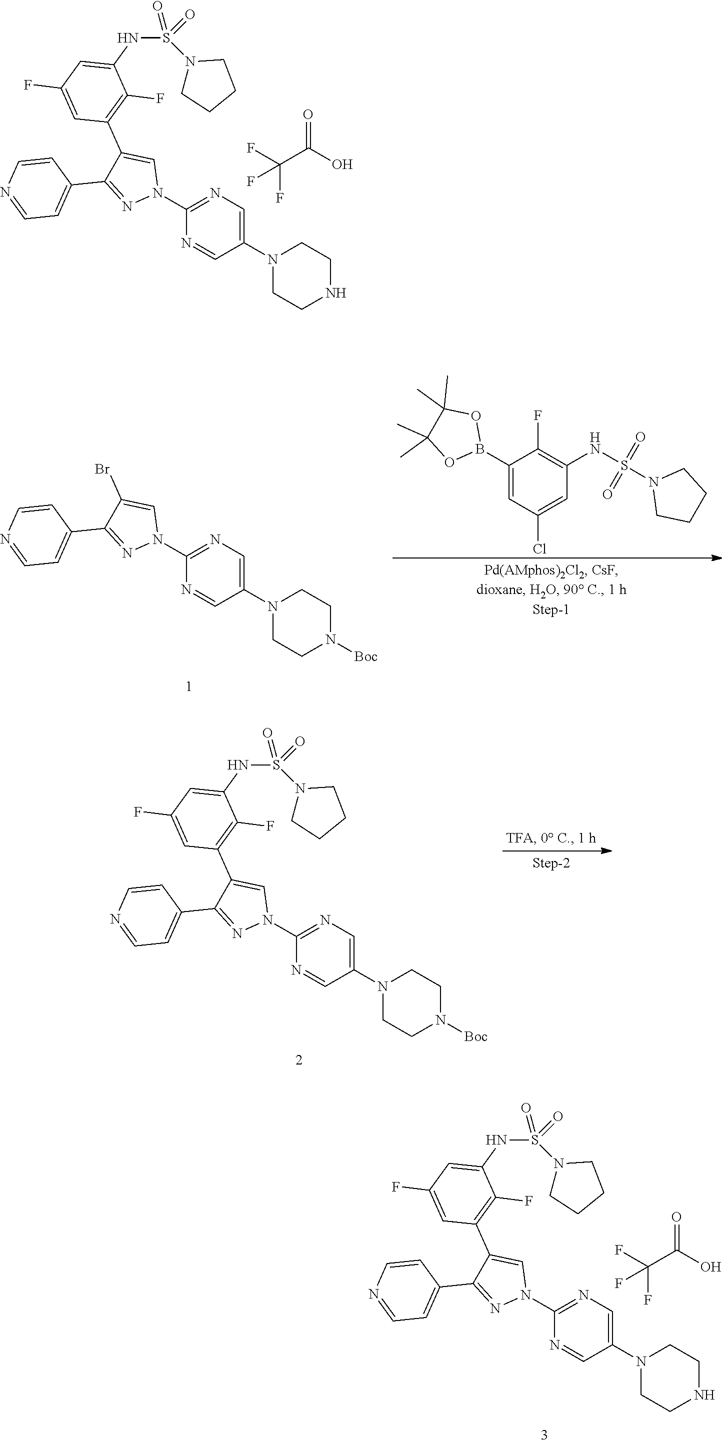

### Step-1: Synthesis of tert-butyl 4-[2-(4-{2,5-difluoro-3-[(pyrrolidine-1-sulfonyl)amino]phenyl}-3-(pyridin-4-yl)pyrazol-1-yl)pyrimidin-5-yl]piperazine-1-carboxylate (2)

To a mixture of tert-butyl 4-{2-[4-bromo-3-(pyridin-4-yl)pyrazol-1-yl]pyrimidin-5-yl}piperazine-1-carboxylate (900 mg, 1.850 mmol, 1 equiv) and N-[2,5-difluoro-3-(4,4,5,5-tetramethyl-1,3,2-dioxaborolan-2-yl)phenyl]pyrrolidine-1-sulfonamide (718 mg, 1.850 mmol, 1 equiv) in dioxane (10 mL) was added Pd(AMphos)₂Cl₂ (131 mg, 0.185 mmol, 0.1 equiv) and CsF (562 mg, 3.700 mmol, 2 equiv) in H₂O (2 mL). The resulting mixture was stirred at 90° C. for one hour under a nitrogen atmosphere. The resulting mixture was then concentrated under vacuum. The residue was purified by silica gel column chromatography eluted with EtOAc:MeOH (9:1) to afford tert-butyl 4-[2-(4-{2,5-difluoro-3-[(pyrrolidine-1-sulfonyl)amino]phenyl}-3-(pyridin-4-yl)pyrazol-1-yl)pyrimidin-5-yl]piperazine-1-carboxylate (700 mg, 45.32%) as a brown solid. LCMS: C₃₁H₃₅F₂N₉O₄S requires: 667.2, found: m/z=668.3 [M+H]⁺.

### Step-2: Synthesis of N-(2,5-difluoro-3-{1-[5-(piperazin-1-yl)pyrimidin-2-yl]-3-(pyridin-4-yl)pyrazol-4-yl}phenyl)pyrrolidine-1-sulfonamide Trifluoroacetic Acid Salt (3)

A mixture of tert-butyl 4-[2-(4-{2,5-difluoro-3-[(pyrrolidine-1-sulfonyl)amino]phenyl}-3-(pyridin-4-yl)pyrazol-1-yl)pyrimidin-5-yl]piperazine-1-carboxylate (700 mg, 1.048 mmol, 1 equiv) in TFA (7 mL) was stirred at 0° C. for one hour. The resulting mixture was then concentrated under vacuum. The crude product was purified by prep-HPLC with the following conditions: Column: XSelect CSH Prep C18 OBD Column, 19×250 mm, 5 μm; Mobile Phase A: Water (0.05% TFA), Mobile Phase B: MeOH; Flow rate: 25 mL/min; Gradient: 30% B to 45% B in 10 min; Wave Length: 254/220 nm to afford N-(2,5-difluoro-3-{1-[5-(piperazin-1-yl)pyrimidin-2-yl]-3-(pyridin-4-yl)pyrazol-4-yl}phenyl)pyrrolidine-1-sulfonamide trifluoroacetic acid salt (320.0 mg, 44.24%) as a yellow solid. LCMS: C₂₆H₂₇F₂N₉O₂S requires: 567.2, found: m/z=568.2 [M+H]⁺. ¹H NMR (400 MHz, Methanol-d₄) δ 8.97-8.92 (m, 1H), 8.84-8.78 (m, 2H), 8.71-8.66 (m, 2H), 8.23-8.17 (m, 2H), 7.47-7.38 (m, 1H), 7.13-7.04 (m, 1H), 3.72-3.65 (m, 4H), 3.51-3.44 (m, 4H), 3.40-3.35 (m, 4H), 1.96-1.85 (m, 4H).

### Synthesis of N-(2,5-difluoro-3-{1-[2-fluoro-4-(piperazin-1-yl)phenyl]-3-(pyridin-4-yl)pyrazol-4-yl}phenyl)pyrrolidine-1-sulfonamide Trifluoroacetic Acid Salt (3)

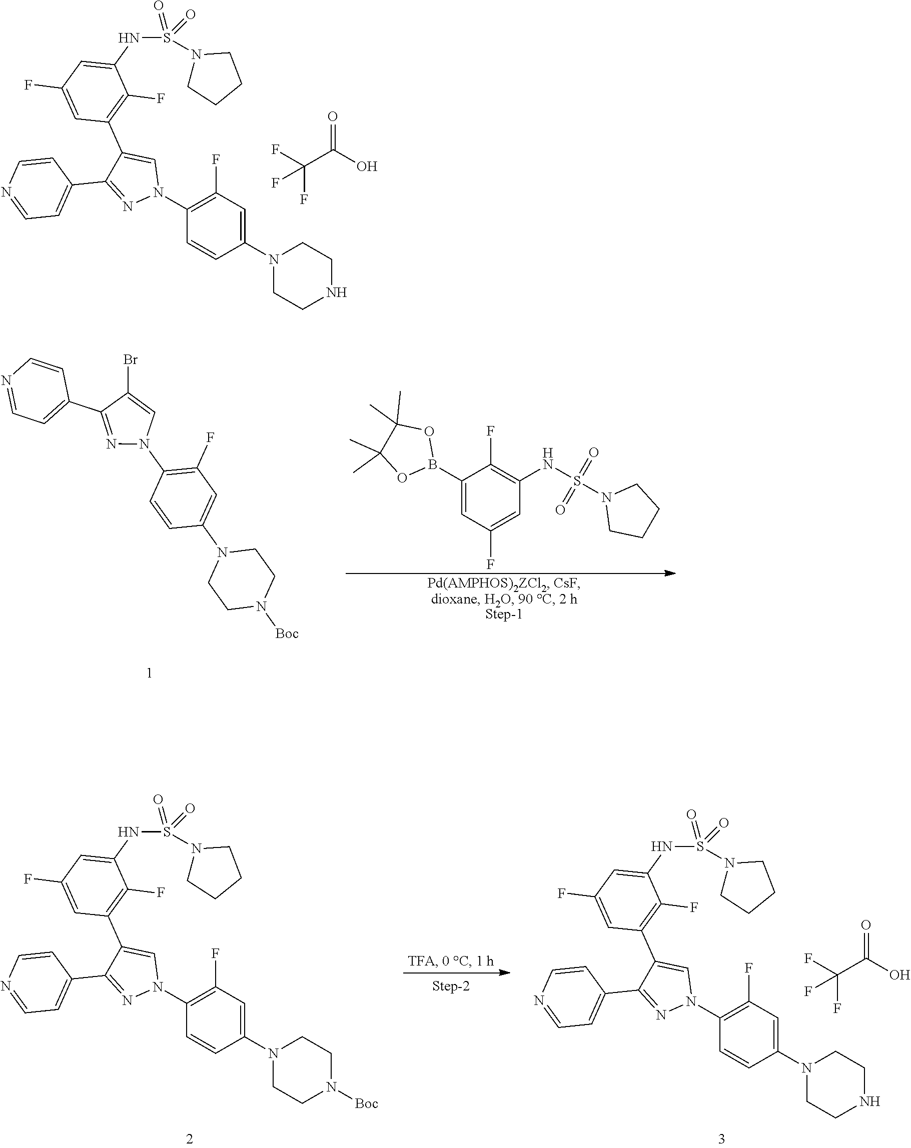

### Step-1: Synthesis of tert-butyl 4-[4-(4-{2,5-difluoro-3-[(pyrrolidine-1-sulfonyl)amino]phenyl}-3-(pyridin-4-yl)pyrazol-1-yl)-3-fluorophenyl]piperazine-1-carboxylate (2)

To a mixture of tert-butyl 4-{4-[4-bromo-3-(pyridin-4-yl)pyrazol-1-yl]-3-fluorophenyl}piperazine-1-carboxylate (900 mg, 1.791 mmol, 1 equiv) and N-[2,5-difluoro-3-(4,4,5,5-tetramethyl-1,3,2-dioxaborolan-2-yl)phenyl]pyrrolidine-1-sulfonamide (695 mg, 1.791 mmol, 1 equiv) in dioxane (10 mL) was added CsF (544 mg, 3.582 mmol, 2 equiv) and Pd(AMPHOS)₂Cl₂ (126 mg, 0.179 mmol, 0.1 equiv) in H₂O (2 mL). The resulting mixture was stirred at 90° C. for 2 h under a nitrogen atmosphere. The resulting mixture was then concentrated under reduced pressure. The residue was purified by silica gel column chromatography eluted with PE:EtOAc (1:3) to afford tert-butyl 4-[4-(4-{2,5-difluoro-3-[(pyrrolidine-1-sulfonyl)amino]phenyl}-3-(pyridin-4-yl)pyrazol-1-yl)-3-fluorophenyl]piperazine-1-carboxylate (600 mg, 48.98%) as a yellow solid. LCMS: C₃₃H₃₆F₃N₇O₄S requires: 683.2, found: m/z=684.3 [M+H]⁺.

### Step-2: Synthesis of N-(2,5-difluoro-3-{1-[2-fluoro-4-(piperazin-1-yl)phenyl]-3-(pyridin-4-yl)pyrazol-4-yl}phenyl)pyrrolidine-1-sulfonamide Trifluoroacetic Acid Salt (3)

A mixture of tert-butyl 4-[4-(4-{2,5-difluoro-3-[(pyrrolidine-1-sulfonyl)amino]phenyl}-3-(pyridin-4-yl)pyrazol-1-yl)-3-fluorophenyl]piperazine-1-carboxylate (600 mg, 0.878 mmol, 1 equiv) in TFA (6 mL) was stirred at 0° C. for one hour. The resulting mixture was then concentrated under reduced pressure. The crude product was purified by prep-HPLC with the following conditions: Column: XSelect CSH Prep C18 OBD Column, 19×250 mm, 5 μm; Mobile Phase A: Water (0.05% TFA), Mobile Phase B: MeOH; Flow rate: 25 mL/min; Gradient: 30% B to 45% B in 12 min; Wave Length: 254/220 nm to afford N-(2,5-difluoro-3-{1-[2-fluoro-4-(piperazin-1-yl)phenyl]-3-(pyridin-4-yl)pyrazol-4-yl}phenyl)pyrrolidine-1-sulfonamide trifluoroacetic acid salt (284.0 mg, 46.39%) as a yellow solid. LCMS: C₂₈H₂₈F₃N₇O₂S requires: 583.2, found: m/z=584.1 [M+H]⁺. ¹H NMR (300 MHz, Methanol-d₄) δ 8.80-8.72 (m, 2H), 8.41-8.34 (m, 1H), 8.16-8.08 (m, 2H), 7.90-7.78 (m, 1H), 7.47-7.35 (m, 1H), 7.14-7.01 (m, 3H), 3.65-3.55 (m, 4H), 3.47-3.38 (m, 4H), 3.38-3.33 (m, 4H), 1.98-1.84 (m, 4H).

### Synthesis of N-[5-chloro-3-(1-{4-[(3R,5R)-3,5-dimethylpiperazin-1-yl]-2-fluorophenyl}-3-(pyridin-4-yl)pyrazol-4-yl)-2-fluorophenyl]pyrrolidine-1-sulfonamide; Trifluoroacetic Acid (6)

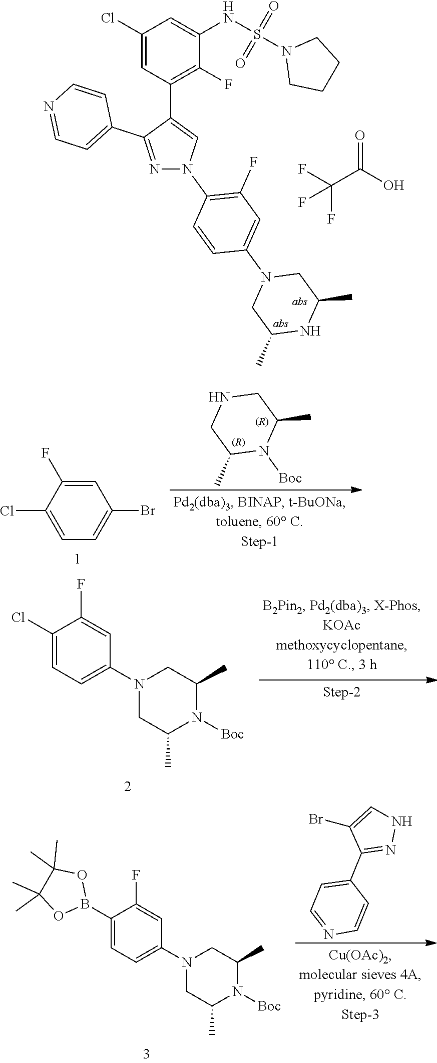

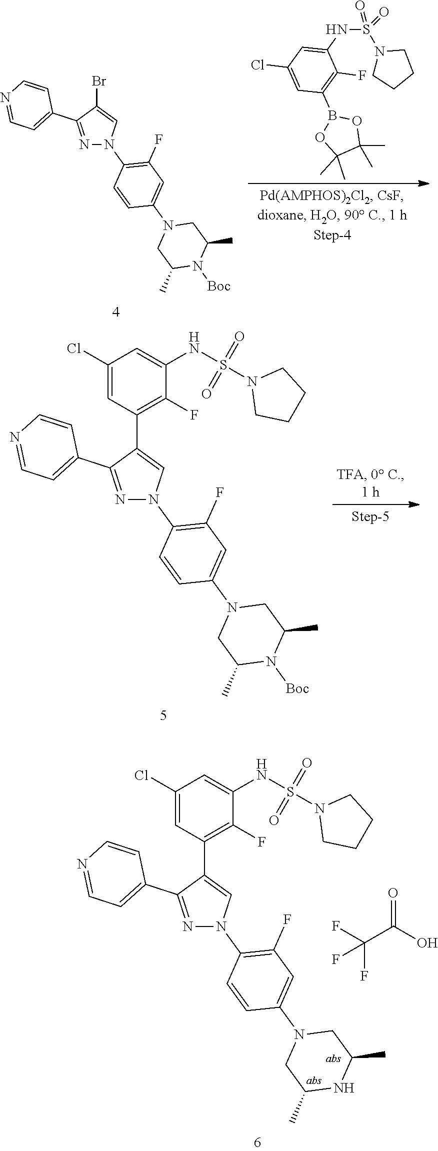

### Step-1: Synthesis of tert-butyl (2R,6R)-4-(4-chloro-3-fluorophenyl)-2,6-dimethylpiperazine-1-carboxylate (2)

To a mixture of 4-bromo-1-chloro-2-fluorobenzene (4.9 g, 23.331 mmol, 1 equiv) and tert-butyl (2R,6R)-2,6-dimethylpiperazine-1-carboxylate (5 g, 23.331 mmol, 1 equiv) in toluene (60 mL) was added Pd₂(dba)₃ (214 mg, 0.233 mmol, 0.01 equiv), t-BuONa (3.4 g, 32.663 mmol, 1.4 equiv), and BINAP (291 mg, 0.467 mmol, 0.02 equiv). The resulting mixture was stirred at 60° C. overnight under a nitrogen atmosphere. The resulting mixture was then concentrated under vacuum. The residue was purified by silica gel column chromatography eluted with PE:EtOAc (10:1) to afford tert-butyl (2R,6R)-4-(4-chloro-3-fluorophenyl)-2,6-dimethylpiperazine-1-carboxylate (5 g, 56.26%) as a brown solid. LCMS: C₁₇H₂₄ClFN₂O₂ requires: 342.1, found: m/z=343.2 [M+H]⁺.

### Step-2: Synthesis of tert-butyl (2R,6R)-4-[3-fluoro-4-(4,4,5,5-tetramethyl-1,3,2-dioxaborolan-2-yl)phenyl]-2,6-dimethylpiperazine-1-carboxylate (3)

To a mixture of tert-butyl (2R,6R)-4-(4-chloro-3-fluorophenyl)-2,6-dimethylpiperazine-1-carboxylate (5 g, 14.584 mmol, 1 equiv) and 4,4,5,5-tetramethyl-2-(4,4,5,5-tetramethyl-1,3,2-dioxaborolan-2-yl)-1,3,2-dioxaborolane (B₂Pin₂) (5.6 g, 21.876 mmol, 1.5 equiv) in methoxycyclopentane (70 mL) was added Pd₂(dba)₃ (401 mg, 0.438 mmol, 0.03 equiv), KOAc (4.3 g, 43.752 mmol, 3 equiv), and XPhos (417 mg, 0.875 mmol, 0.06 equiv). The resulting mixture was stirred at 110° C. for 3 h under a nitrogen atmosphere. The resulting mixture was then concentrated under vacuum. The residue was purified by silica gel column chromatography eluted with PE:EtOAc (9:1) to afford tert-butyl (2R,6R)-4-[3-fluoro-4-(4,4,5,5-tetramethyl-1,3,2-dioxaborolan-2-yl)phenyl]-2,6-dimethylpiperazine-1-carboxylate (5 g, 71.04%) as a brown solid. LCMS: C₂₃H₃₆BFN₂O₄ requires: 434.3, found: m/z=435.3 [M+H]⁺.

### Step-3: Synthesis of tert-butyl (2R,6R)-4-{4-[4-bromo-3-(pyridin-4-yl)pyrazol-1-yl]-3-fluorophenyl}-2,6-dimethylpiperazine-1-carboxylate (4)

To a mixture of tert-butyl (2R,6R)-4-[3-fluoro-4-(4,4,5,5-tetramethyl-1,3,2-dioxaborolan-2-yl)phenyl]-2,6-dimethylpiperazine-1-carboxylate (3.5 g, 8.058 mmol, 1 equiv) and 4-(4-bromo-1H-pyrazol-3-yl)pyridine (2 g, 8.864 mmol, 1.1 equiv) in pyridine (40 mL) was added Cu(OAc)₂ (2.9 g, 16.116 mmol, 2 equiv) and molecular sieves type 4A (3.5 g). The resulting mixture was stirred at 60° C. overnight under an oxygen atmosphere. The resulting mixture was then extracted with EtOAc. The combined organic layers were washed with water and dried over anhydrous Na₂SO₄. After filtration, the filtrate was concentrated under reduced pressure. The residue was purified by silica gel column chromatography eluted with PE:EtOAc (1:1) to afford tert-butyl (2R,6R)-4-{4-[4-bromo-3-(pyridin-4-yl)pyrazol-1-yl]-3-fluorophenyl}-2,6-dimethylpiperazine-1-carboxylate (1.5 g, 31.58%) as an off-white solid. LCMS: C₂₅H₂₉BrFN₅O₂ requires: 529.2, found: m/z=530.4 [M+H]⁺.

### Step-4: Synthesis of tert-butyl (2R,6R)-4-[4-(4-{5-chloro-2-fluoro-3-[(pyrrolidine-1-sulfonyl)amino]phenyl}-3-(pyridin-4-yl)pyrazol-1-yl)-3-fluorophenyl]-2,6-dimethylpiperazine-1-carboxylate (5)

To a mixture of tert-butyl (2R,6R)-4-{4-[4-bromo-3-(pyridin-4-yl)pyrazol-1-yl]-3-fluorophenyl}-2,6-dimethylpiperazine-1-carboxylate (500 mg, 0.943 mmol, 1 equiv) and N-[5-chloro-2-fluoro-3-(4,4,5,5-tetramethyl-1,3,2-dioxaborolan-2-yl)phenyl]pyrrolidine-1-sulfonamide (382 mg, 0.943 mmol, 1 equiv) in dioxane (10 mL) was added Pd(AMPHOS)₂Cl₂ (67 mg, 0.094 mmol, 0.1 equiv) and CsF (286 mg, 1.886 mmol, 2 equiv) in H₂O (2 mL). The resulting mixture was stirred at 90° C. for one hour under a nitrogen atmosphere. The resulting mixture was then concentrated under vacuum. The residue was purified by silica gel column chromatography eluted with PE:EtOAc (1:2). The residue was then purified by trituration with EtOAc. This resulted in tert-butyl (2R,6R)-4-[4-(4-{5-chloro-2-fluoro-3-[(pyrrolidine-1-sulfonyl)amino]phenyl}-3-(pyridin-4-yl)pyrazol-1-yl)-3-fluorophenyl]-2,6-dimethylpiperazine-1-carboxylate (190 mg, 26.29%) as a white solid. LCMS: C₃₅H₄₀ClF₂N₇O₄S requires: 727.2, found: m/z=728.3 [M+H]⁺.

### Step-5: Synthesis of N-[5-chloro-3-(1-{4-[(3R,5R)-3,5-dimethylpiperazin-1-yl]-2-fluorophenyl}-3-(pyridin-4-yl)pyrazol-4-yl)-2-fluorophenyl]pyrrolidine-1-sulfonamide Trifluoroacetic Acid Salt (6)

A mixture of tert-butyl (2R,6R)-4-[4-(4-{5-chloro-2-fluoro-3-[(pyrrolidine-1-sulfonyl)amino]phenyl}-3-(pyridin-4-yl)pyrazol-1-yl)-3-fluorophenyl]-2,6-dimethylpiperazine-1-carboxylate (190 mg, 0.261 mmol, 1 equiv) in TFA (2 mL) was stirred at 0° C. for one hour. The resulting mixture was concentrated under vacuum and lyophilized. This resulted in N-[5-chloro-3-(1-{4-[(3R,5R)-3,5-dimethylpiperazin-1-yl]-2-fluorophenyl}-3-(pyridin-4-yl)pyrazol-4-yl)-2-fluorophenyl]pyrrolidine-1-sulfonamide trifluoroacetic acid salt (133.3 mg, crude) as a yellow solid. LCMS: C₃₀H₃₂ClF₂N₇O₂S requires: 627.2, found: m/z=628.2 [M+H]⁺. ¹H NMR (400 MHz, Methanol-d₄) δ 8.80-8.71 (m, 2H), 8.41-8.32 (m, 1H), 8.15-8.09 (m, 2H), 7.87-7.78 (m, 1H), 7.67-7.60 (m, 1H), 7.37-7.30 (m, 1H), 7.13-7.00 (m, 2H), 3.90-3.78 (m, 2H), 3.70-3.61 (m, 2H), 3.41-3.33 (m, 3H), 3.33-3.28 (m, 3H), 1.96-1.85 (m, 4H), 1.52-1.46 (m, 6H).

### Synthesis of N-[5-chloro-2-fluoro-3-(1-{2-fluoro-4-[(2S)-2-methylpiperazin-1-yl]phenyl}-3-(pyridin-4-yl)pyrazol-4-yl)phenyl]pyrrolidine-1-sulfonamide Trifluoroacetic Acid Salt (6)

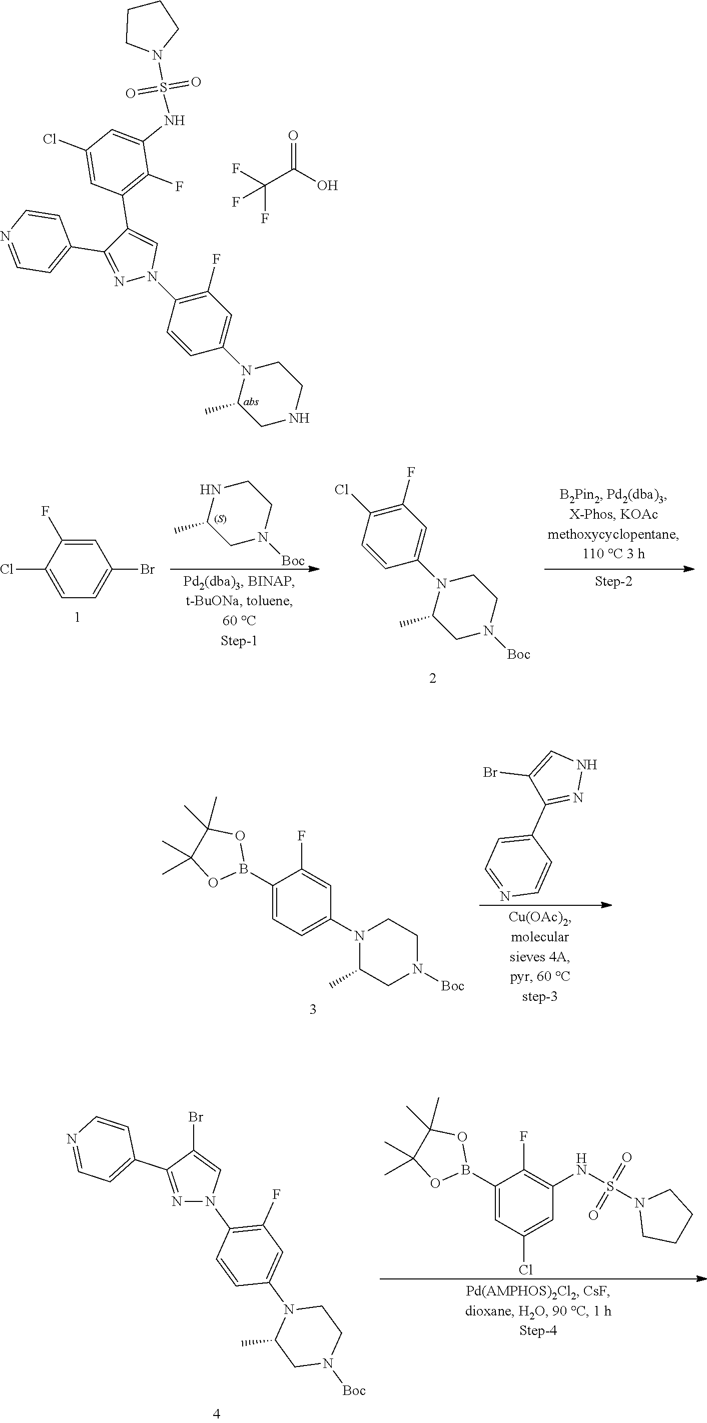

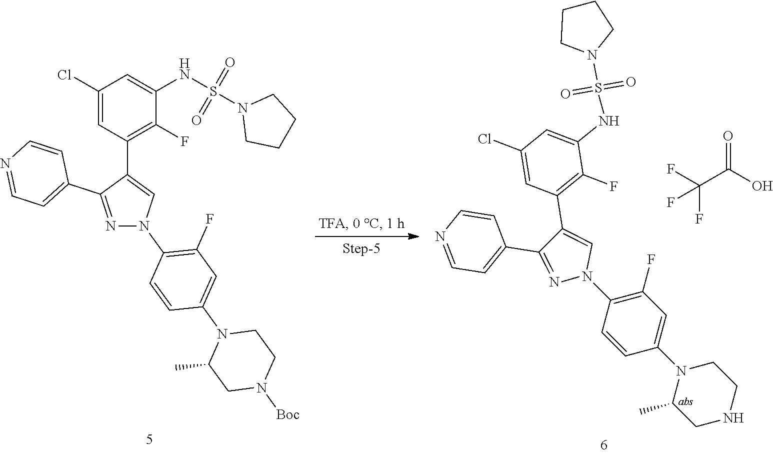 [SMILES: C[C@H]1CN(C(=O)OC(C)(C)C)CCN1c1ccc(-n2cc(-c3cc(Cl)cc(NS(=O)(=O)N4CCCC4)c3F)c(-c3ccncc3)n2)c(F)c1 ; C[C@H]1CNCCN1c1ccc(-n2cc(-c3cc(Cl)cc(NS(=O)(=O)N4CCCC4)c3F)c(-c3ccncc3)n2)c(F)c1 ; O=C(O)C(F)(F)F]

### Step-1: Synthesis of tert-butyl (3S)-4-(4-chloro-3-fluorophenyl)-3-methylpiperazine-1-carboxylate (2)

To a mixture of 4-bromo-1-chloro-2-fluorobenzene (8 g, 38.197 mmol, 1 equiv) and tert-butyl (3S)-3-methylpiperazine-1-carboxylate (7.7 g, 38.197 mmol, 1 equiv) in toluene (100 mL) was added Pd₂(dba)₃ (350 mg, 0.382 mmol, 0.01 equiv), t-BuONa (5.1 g, 53.476 mmol, 1.4 equiv), and BINAP (476 mg, 0.764 mmol, 0.02 equiv). The resulting mixture was stirred at 60° C. overnight under a nitrogen atmosphere. The resulting mixture was then concentrated under vacuum. The residue was purified by silica gel column chromatography eluted with PE:EtOAc (5:1) to afford tert-butyl (3S)-4-(4-chloro-3-fluorophenyl)-3-methylpiperazine-1-carboxylate (8 g, 37.82%) as a yellow oil. LCMS: C₁₆H₂₂ClFN₂O₂ requires: 328.1, found: m/z=329.2 [M+H]⁺.

### Step-2: Synthesis of tert-butyl (3S)-4-[3-fluoro-4-(4,4,5,5-tetramethyl-1,3,2-dioxaborolan-2-yl)phenyl]-3-methylpiperazine-1-carboxylate (3)

To a mixture of tert-butyl (3S)-4-(4-chloro-3-fluorophenyl)-3-methylpiperazine-1-carboxylate (5 g, 15.206 mmol, 1 equiv) and 4,4,5,5-tetramethyl-2-(4,4,5,5-tetramethyl-1,3,2-dioxaborolan-2-yl)-1,3,2-dioxaborolane (B₂Pin₂) (5.8 g, 22.809 mmol, 1.5 equiv) in methoxycyclopentane (70 mL) was added Pd₂(dba)₃ (418 mg, 0.456 mmol, 0.03 equiv), KOAc (4.5 g, 45.618 mmol, 3 equiv), and XPhos (435 mg, 0.912 mmol, 0.06 equiv). The resulting mixture was stirred at 110° C. for 3 h under a nitrogen atmosphere. The resulting mixture was then concentrated under vacuum. The residue was purified by silica gel column chromatography eluted with PE:EtOAc (9:1) to afford tert-butyl (3S)-4-[3-fluoro-4-(4,4,5,5-tetramethyl-1,3,2-dioxaborolan-2-yl)phenyl]-3-methylpiperazine-1-carboxylate (5 g, 70.40%) as a brown solid. LCMS: C₂₂H₃₄BFN₂O₄ requires: 420.2, found: m/z=421.3 [M+H]⁺.

### Step-3: Synthesis of tert-butyl (3S)-4-{4-[4-bromo-3-(pyridin-4-yl)pyrazol-1-yl]-3-fluorophenyl}-3-methylpiperazine-1-carboxylate (4)

To a mixture of tert-butyl (3S)-4-[3-fluoro-4-(4,4,5,5-tetramethyl-1,3,2-dioxaborolan-2-yl)phenyl]-3-methylpiperazine-1-carboxylate (2.5 g, 5.948 mmol, 1 equiv) and 4-(4-bromo-1H-pyrazol-3-yl)pyridine (1.5 g, 6.543 mmol, 1.1 equiv) in pyridine (30 mL) was added Cu(OAc)₂ (2.2 g, 11.896 mmol, 2 equiv) and molecular sieves type 4A (2.5 g). The resulting mixture was stirred at 60° C. overnight under an oxygen atmosphere. The resulting mixture was then extracted with EtOAc. The combined organic layers were washed with water and dried over anhydrous Na₂SO₄. After filtration, the filtrate was concentrated under reduced pressure. The residue was purified by silica gel column chromatography eluted with PE:EtOAc (1:1). The residue was then purified by trituration with EtOAc. This resulted in tert-butyl (3S)-4-{4-[4-bromo-3-(pyridin-4-yl)pyrazol-1-yl]-3-fluorophenyl}-3-methylpiperazine-1-carboxylate (1 g, 29.30%) as an off-white semi-solid. LCMS: C₂₄H₂₇BrFN₅O₂ requires: 515.1, found: m/z=516.3 [M+H]⁺.

### Step-4: Synthesis of tert-butyl (3S)-4-[4-(4-{5-chloro-2-fluoro-3-[(pyrrolidine-1-sulfonyl)amino]phenyl}-3-(pyridin-4-yl)pyrazol-1-yl)-3-fluorophenyl]-3-methylpiperazine-1-carboxylate (5)

To a mixture of tert-butyl (3S)-4-{4-[4-bromo-3-(pyridin-4-yl)pyrazol-1-yl]-3-fluorophenyl}-3-methylpiperazine-1-carboxylate (500 mg, 0.968 mmol, 1 equiv) and N-[5-chloro-2-fluoro-3-(4,4,5,5-tetramethyl-1,3,2-dioxaborolan-2-yl)phenyl]pyrrolidine-1-sulfonamide (392 mg, 0.968 mmol, 1 equiv) in dioxane (10 mL) was added Pd(AMPHOS)₂Cl₂ (69 mg, 0.097 mmol, 0.1 equiv) and CsF (294 mg, 1.936 mmol, 2 equiv) in H₂O (2 mL). The resulting mixture was stirred at 90° C. for one hour under a nitrogen atmosphere. The resulting mixture was then concentrated under vacuum. The residue was purified by silica gel column chromatography eluted with PE:EtOAc (1:2). The residue was purified by trituration with EtOAc. This resulted in tert-butyl (3S)-4-[4-(4-{5-chloro-2-fluoro-3-[(pyrrolidine-1-sulfonyl)amino]phenyl}-3-(pyridin-4-yl)pyrazol-1-yl)-3-fluorophenyl]-3-methylpiperazine-1-carboxylate (200 mg, 27.48%) as a white solid. LCMS: C₃₄H₃₈ClF₂N₇O₄S requires: 713.2, found: m/z=714.3 [M+H]⁺.

### Step-5: Synthesis of N-[5-chloro-2-fluoro-3-(1-{2-fluoro-4-[(2S)-2-methylpiperazin-1-yl]phenyl}-3-(pyridin-4-yl)pyrazol-4-yl)phenyl]pyrrolidine-1-sulfonamide Trifluoroacetic Acid Salt (6)

A mixture of tert-butyl (3S)-4-[4-(4-{5-chloro-2-fluoro-3-[(pyrrolidine-1-sulfonyl)amino]phenyl}-3-(pyridin-4-yl)pyrazol-1-yl)-3-fluorophenyl]-3-methylpiperazine-1-carboxylate (200 mg, 0.280 mmol, 1 equiv) in TFA (2 mL) was stirred at 0° C. for one hour. The resulting mixture was then concentrated under vacuum and lyophilized. This resulted in N-[5-chloro-2-fluoro-3-(1-{2-fluoro-4-[(2S)-2-methylpiperazin-1-yl]phenyl}-3-(pyridin-4-yl)pyrazol-4-yl)phenyl]pyrrolidine-1-sulfonamide trifluoroacetic acid salt (144.3 mg, crude) as a yellow solid. LCMS: C₂₉H₃₀ClF₂N₇O₂S requires: 613.1, found: m/z=614.2 [M+H]⁺. ¹H NMR (400 MHz, Methanol-d₄) δ 8.78-8.72 (m, 2H), 8.41-8.36 (m, 1H), 8.11-8.04 (m, 2H), 7.89-7.80 (m, 1H), 7.67-7.29 (m, 2H), 7.12-7.00 (m, 2H), 4.39-4.31 (m, 1H), 3.75-3.66 (m, 1H), 3.54-3.34 (m, 4H), 3.34-3.33 (m, 3H), 3.33-3.24 (m, 2H), 1.96-1.85 (m, 4H), 1.36-1.23 (m, 3H).

### Synthesis of N-[5-chloro-2-fluoro-3-(1-{2-fluoro-4-[(2R)-2-methylpiperazin-1-yl]phenyl}-3-(pyridin-4-yl)pyrazol-4-yl)phenyl]pyrrolidine-1-sulfonamide Trifluoroacetic Acid Salt (6)

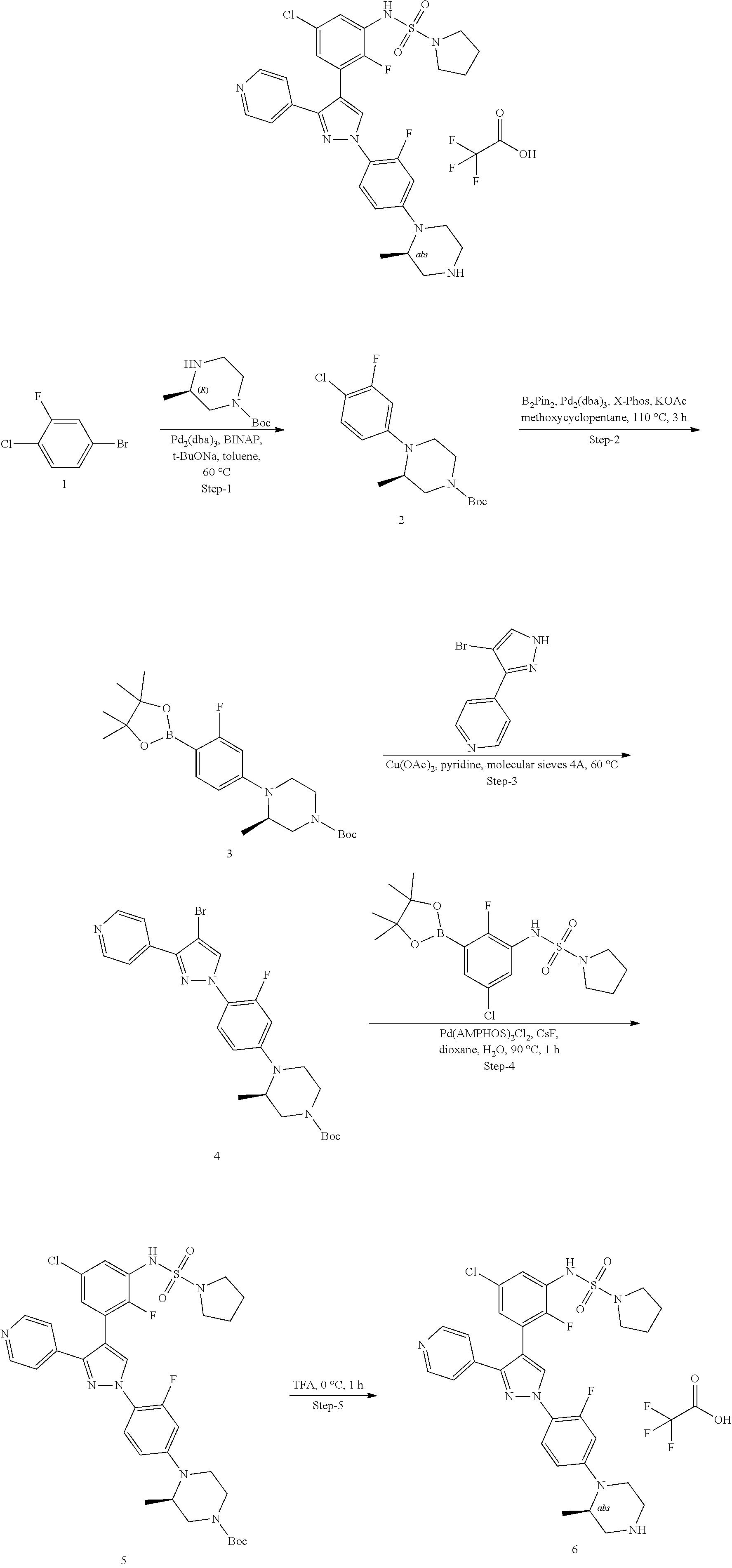

### Step-1: Synthesis of tert-butyl (3R)-4-(4-chloro-3-fluorophenyl)-3-methylpiperazine-1-carboxylate (2)

To a mixture of 4-bromo-1-chloro-2-fluorobenzene (8 g, 38.197 mmol, 1 equiv) and tert-butyl (3R)-3-methylpiperazine-1-carboxylate (7.65 g, 38.197 mmol, 1 equiv) in toluene (80 mL) was added BINAP (0.48 g, 0.764 mmol, 0.02 equiv), t-BuONa (5.14 g, 53.476 mmol, 1.4 equiv), and Pd₂(dba)₃ (0.35 g, 0.382 mmol, 0.01 equiv). The resulting mixture was stirred at 60° C. overnight under a nitrogen atmosphere. The resulting mixture was then concentrated under reduced pressure. The residue was purified by silica gel column chromatography eluted with PE:EtOAc (4:1) to afford tert-butyl (3R)-4-(4-chloro-3-fluorophenyl)-3-methylpiperazine-1-carboxylate (6 g, 47.77%) as a yellow oil. LCMS: C₁₆H₂₂ClFN₂O₂ requires: 328.1, found: m/z=329.0 [M+H]⁺.

### Step-2: Synthesis of tert-butyl (3R)-4-[3-fluoro-4-(4,4,5,5-tetramethyl-1,3,2-dioxaborolan-2-yl)phenyl]-3-methylpiperazine-1-carboxylate (3)

To a mixture of tert-butyl (3R)-4-(4-chloro-3-fluorophenyl)-3-methylpiperazine-1-carboxylate (6 g, 18.248 mmol, 1 equiv) and 4,4,5,5-tetramethyl-2-(4,4,5,5-tetramethyl-1,3,2-dioxaborolan-2-yl)-1,3,2-dioxaborolane (B₂Pin₂) (6.95 g, 27.372 mmol, 1.5 equiv) in methoxycyclopentane (60 mL) was added AcOK (5.37 g, 54.744 mmol, 3 equiv), XPhos (0.52 g, 1.095 mmol, 0.06 equiv), and Pd₂(dba)₃ (0.5 g, 0.547 mmol, 0.03 equiv). The resulting mixture was stirred at 110° C. for 3 h under a nitrogen atmosphere. The resulting mixture was then concentrated under reduced pressure. The residue was purified by silica gel column chromatography eluted with PE:EtOAc (8:1) to afford tert-butyl (3R)-4-[3-fluoro-4-(4,4,5,5-tetramethyl-1,3,2-dioxaborolan-2-yl)phenyl]-3-methylpiperazine-1-carboxylate (6 g, 78.23%) as a yellow solid. LCMS: C₂₂H₃₄BFN₂O₄ requires: 420.3, found: m/z=421.1 [M+H]⁺.

### Step-3: Synthesis of tert-butyl (3R)-4-{4-[4-bromo-3-(pyridin-4-yl)pyrazol-1-yl]-3-fluorophenyl}-3-methylpiperazine-1-carboxylate (4)

To a mixture of tert-butyl (3R)-4-[3-fluoro-4-(4,4,5,5-tetramethyl-1,3,2-dioxaborolan-2-yl)phenyl]-3-methylpiperazine-1-carboxylate (3 g, 7.137 mmol, 1 equiv) and 4-(4-bromo-1H-pyrazol-3-yl)pyridine (1.76 g, 7.851 mmol, 1.1 equiv) in pyridine (30 mL) was added Cu(OAc)₂ (2.59 g, 14.274 mmol, 2 equiv) and molecular sieves (4A) (3 g). The resulting mixture was stirred at 60° C. overnight under an oxygen atmosphere. The resulting mixture was then extracted with EtOAc. The combined organic layers were washed with water and dried over anhydrous Na₂SO₄. After filtration, the filtrate was concentrated under reduced pressure. The residue was purified by silica gel column chromatography eluted with PE:EtOAc (1:1) to afford tert-butyl (3R)-4-{4-[4-bromo-3-(pyridin-4-yl)pyrazol-1-yl]-3-fluorophenyl}-3-methylpiperazine-1-carboxylate (2 g, 54.26%) as a white solid. LCMS: C₂₄H₂₇BrFN₅O₂ requires: 515.1, found: m/z=516.1 [M+H]⁺.

### Step-4: Synthesis of tert-butyl (3R)-4-[4-(4-{5-chloro-2-fluoro-3-[(pyrrolidine-1-sulfonyl)amino]phenyl}-3-(pyridin-4-yl)pyrazol-1-yl)-3-fluorophenyl]-3-methylpiperazine-1-carboxylate (5)

To a stirred solution of tert-butyl (3R)-4-{4-[4-bromo-3-(pyridin-4-yl)pyrazol-1-yl]-3-fluorophenyl}-3-methylpiperazine-1-carboxylate (500 mg, 0.968 mmol, 1 equiv) and N-[5-chloro-2-fluoro-3-(4,4,5,5-tetramethyl-1,3,2-dioxaborolan-2-yl)phenyl]pyrrolidine-1-sulfonamide (391 mg, 0.968 mmol, 1 equiv) in dioxane (5 mL) was added Pd(AMPHOS)₂Cl₂ (68 mg, 0.097 mmol, 0.1 equiv) and CsF (294 mg, 1.936 mmol, 2 equiv) in H₂O (1 mL). The resulting mixture was stirred at 90° C. for one hour under a nitrogen atmosphere. The resulting mixture was then concentrated under reduced pressure. The residue was purified by silica gel column chromatography eluted with PE:EtOAc (1:4) to afford tert-butyl (3R)-4-[4-(4-{5-chloro-2-fluoro-3-[(pyrrolidine-1-sulfonyl)amino]phenyl}-3-(pyridin-4-yl)pyrazol-1-yl)-3-fluorophenyl]-3-methylpiperazine-1-carboxylate (450 mg, 65.07%) as a white solid. LCMS: C₃₄H₃₈ClF₂N₇O₄S requires: 713.2, found: m/z=714.0 [M+H]⁺.

### Step-5: Synthesis of N-[5-chloro-2-fluoro-3-(1-{2-fluoro-4-[(2R)-2-methylpiperazin-1-yl]phenyl}-3-(pyridin-4-yl)pyrazol-4-yl)phenyl]pyrrolidine-1-sulfonamide Trifluoroacetic Acid Salt (6)

A mixture of tert-butyl (3R)-4-[4-(4-{5-chloro-2-fluoro-3-[(pyrrolidine-1-sulfonyl)amino]phenyl}-3-(pyridin-4-yl)pyrazol-1-yl)-3-fluorophenyl]-3-methylpiperazine-1-carboxylate (450 mg, 0.630 mmol, 1 equiv) in TFA (5 mL) was stirred at 0° C. for one hour. The resulting mixture was then concentrated under reduced pressure. The crude product was purified by prep-HPLC with the following conditions: Column: Xcelect CSH F-pheny OBD Column 19×250 mm, 5 μm; Mobile Phase A: Water (0.05% TFA), Mobile Phase B: MeOH; Flow rate: 25 mL/min; Gradient: 30% B to 45% B in 12 min; Wave Length: 254/220 nm to afford N-[5-chloro-2-fluoro-3-(1-{2-fluoro-4-[(2R)-2-methylpiperazin-1-yl]phenyl}-3-(pyridin-4-yl)pyrazol-4-yl)phenyl]pyrrolidine-1-sulfonamide trifluoroacetic acid salt (149.4 mg, 32.57%) as a yellow solid. LCMS: C₂₉H₃₀ClF₂N₇O₂S requires: 613.2, found: m/z=614.3 [M+H]⁺. ¹H NMR (400 MHz, Methanol-d₄) δ 8.78-8.69 (m, 2H), 8.41-8.36 (m, 1H), 8.11-8.01 (m, 2H), 7.89-7.80 (m, 1H), 7.67-7.60 (m, 1H), 7.36-7.29 (m, 1H), 7.12-7.01 (m, 2H), 4.39-4.30 (m, 1H), 3.75-3.65 (m, 1H), 3.54-3.44 (m, 2H), 3.43-3.38 (m, 2H), 3.37-3.34 (m, 2H), 3.33 (s, 2H), 3.32 (s, 1H), 1.98-1.84 (m, 4H), 1.33-1.20 (m, 3H).

### Synthesis of N-[3-(1-{4-[(3R,5R)-3,5-dimethylpiperazin-1-yl]-2-fluorophenyl}-3-(pyridin-4-yl)pyrazol-4-yl)-2,5-difluorophenyl]pyrrolidine-1-sulfonamide Trifluoroacetic Acid Salt (3)

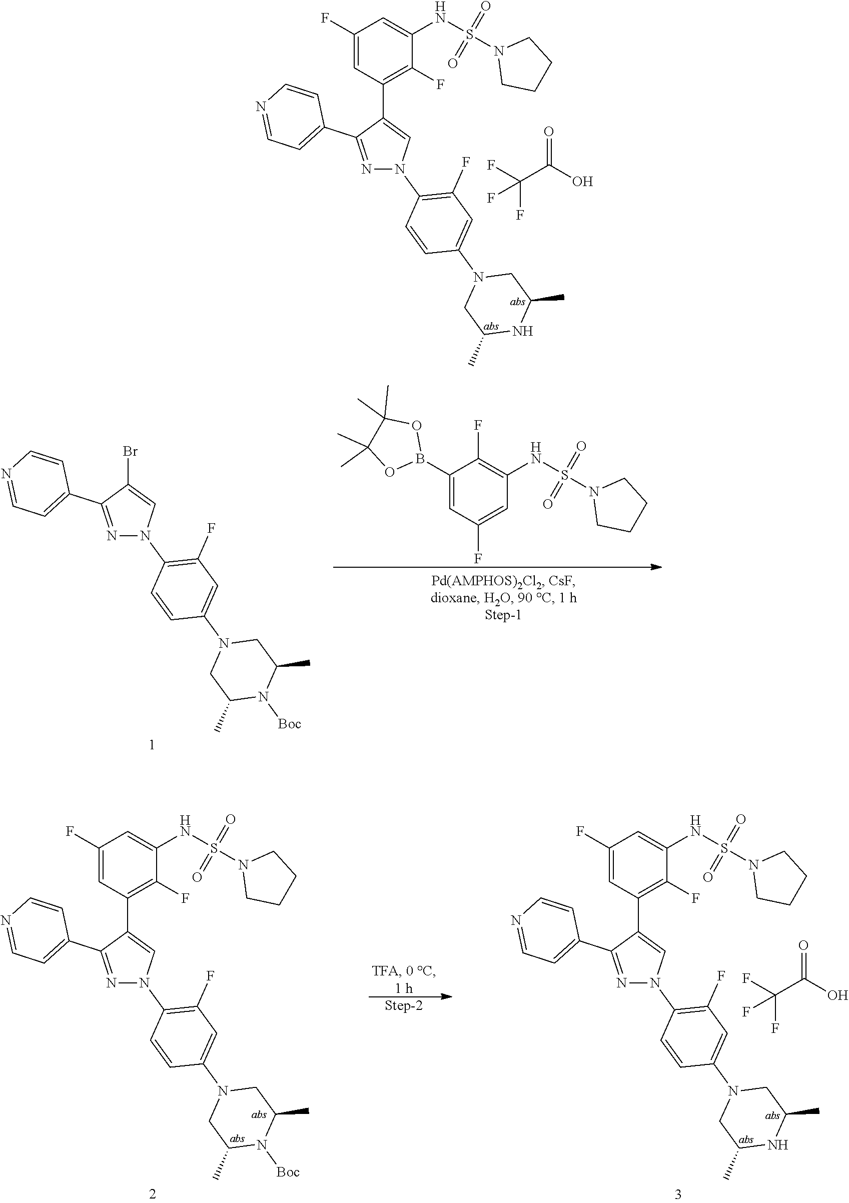

### Step-1: Synthesis of tert-butyl (2R,6R)-4-[4-(4-{2,5-difluoro-3-[(pyrrolidine-1-sulfonyl)amino]phenyl}-3-(pyridin-4-yl)pyrazol-1-yl)-3-fluorophenyl]-2,6-dimethylpiperazine-1-carboxylate (2)

To a mixture of tert-butyl (2R,6R)-4-{4-[4-bromo-3-(pyridin-4-yl)pyrazol-1-yl]-3-fluorophenyl}-2,6-dimethylpiperazine-1-carboxylate (500 mg, 0.943 mmol, 1 equiv) and N-[2,5-difluoro-3-(4,4,5,5-tetramethyl-1,3,2-dioxaborolan-2-yl)phenyl]pyrrolidine-1-sulfonamide (366 mg, 0.943 mmol, 1 equiv) in dioxane (7.5 mL) was added Pd(AMPHOS)₂Cl₂ (67 mg, 0.094 mmol, 0.1 equiv) and CsF (286 mg, 1.886 mmol, 2 equiv) in H₂O (1.5 mL). The resulting mixture was stirred at 90° C. for one hour under a nitrogen atmosphere. The resulting mixture was then concentrated under reduced pressure. The residue was purified by silica gel column chromatography eluted with PE:EtOAc (1:2) to afford tert-butyl (2R,6R)-4-[4-(4-{2,5-difluoro-3-[(pyrrolidine-1-sulfonyl)amino]phenyl}-3-(pyridin-4-yl)pyrazol-1-yl)-3-fluorophenyl]-2,6-dimethylpiperazine-1-carboxylate (300 mg, 42.48%) as a white solid. LCMS: C₃₅H₄₀F₃N₇O₄S requires: 711.3, found: m/z=712.3 [M+H]⁺.

### Step-2: Synthesis of N-[3-(1-{4-[(3R,5R)-3,5-dimethylpiperazin-1-yl]-2-fluorophenyl}-3-(pyridin-4-yl)pyrazol-4-yl)-2,5-difluorophenyl]pyrrolidine-1-sulfonamide Trifluoroacetic Acid Salt (3)

A mixture of tert-butyl (2R,6R)-4-[4-(4-{2,5-difluoro-3-[(pyrrolidine-1-sulfonyl)amino]phenyl}-3-(pyridin-4-yl)pyrazol-1-yl)-3-fluorophenyl]-2,6-dimethylpiperazine-1-carboxylate (30 mg, 0.042 mmol, 1 equiv) in TFA (1 mL) was stirred at 0° C. for one hour. The resulting mixture was concentrated under vacuum and lyophilized. This resulted in N-[3-(1-{4-[(3R,5R)-3,5-dimethylpiperazin-1-yl]-2-fluorophenyl}-3-(pyridin-4-yl)pyrazol-4-yl)-2,5-difluorophenyl]pyrrolidine-1-sulfonamide trifluoroacetic acid salt (16 mg, crude) as a yellow solid. LCMS: C₃₀H₃₂F₃N₇O₂S requires: 611.2, found: m/z=612.3 [M+H]⁺.

¹H NMR (400 MHz, Methanol-d₄) δ 8.83-8.72 (m, 2H), 8.42-8.34 (m, 1H), 8.18-8.09 (m, 2H), 7.90-7.78 (m, 1H), 7.50-7.36 (m, 1H), 7.15-7.00 (m, 3H), 3.92-3.76 (m, 2H), 3.71-3.59 (m, 2H), 3.41-3.34 (m, 2H), 3.33-3.28 (m, 4H), 2.00-1.80 (m, 4H), 1.59-1.42 (m, 6H).

### Synthesis of N-(3-{1-[2,3-difluoro-4-(piperazin-1-yl)phenyl]-3-(pyridin-4-yl)pyrazol-4-yl}-2,5-difluorophenyl)pyrrolidine-1-sulfonamide Trifluoroacetic Acid Salt (6)

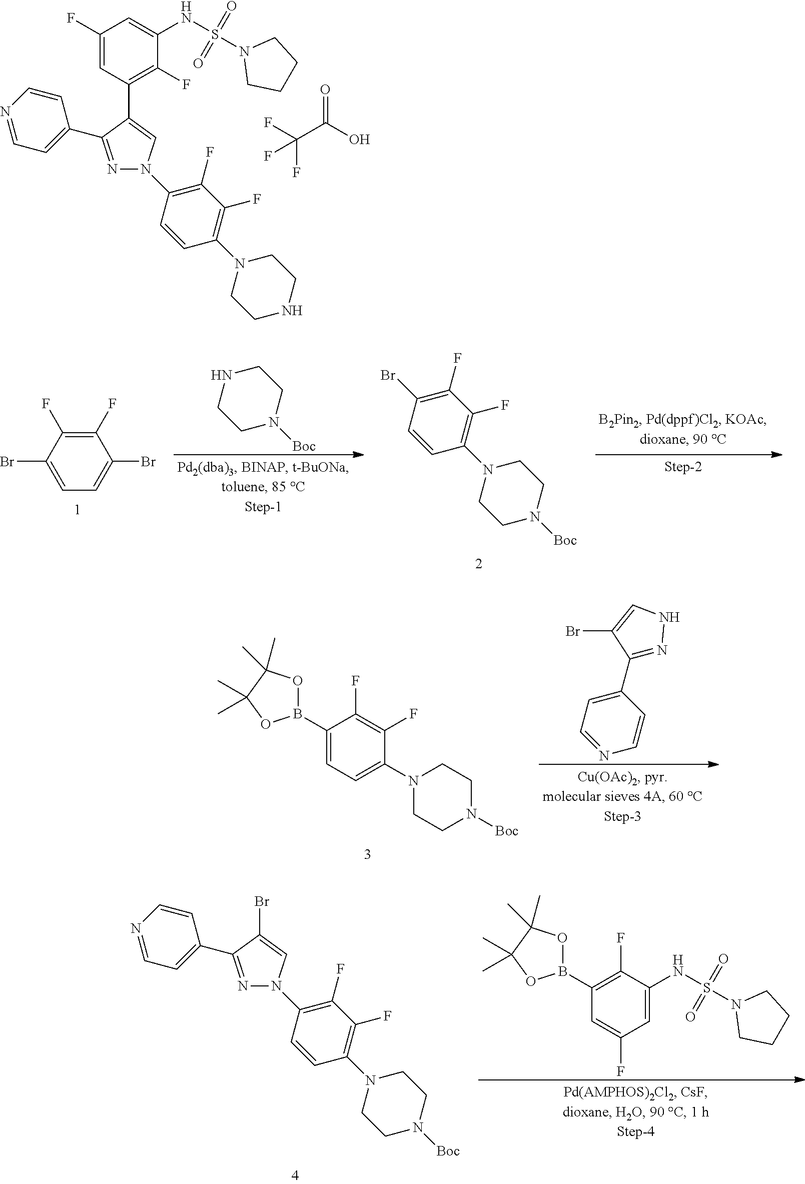

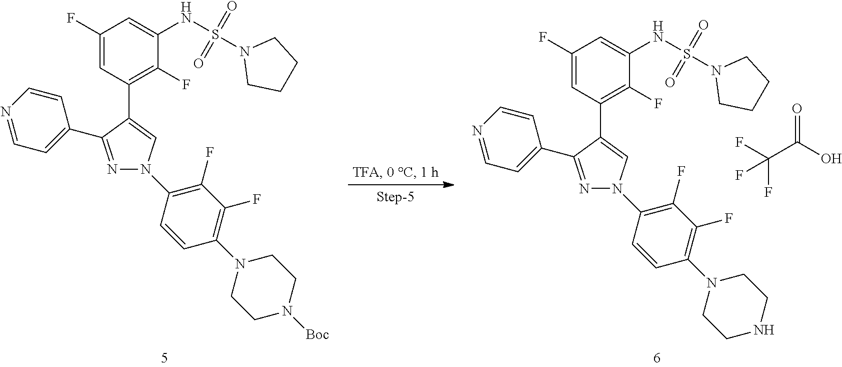

### Step-1: Synthesis of tert-butyl 4-(4-bromo-2,3-difluorophenyl)piperazine-1-carboxylate (2)

To a mixture of 1,4-dibromo-2,3-difluorobenzene (8 g, 29.424 mmol, 1 equiv) and tert-butyl piperazine-1-carboxylate (6.03 g, 32.366 mmol, 1.1 equiv) in toluene (150 mL) was added BINAP (1.1 g, 1.765 mmol, 0.06 equiv), t-BuONa (3.39 g, 35.309 mmol, 1.2 equiv), and Pd₂(dba)₃ (1.62 g, 1.769 mmol, 0.06 equiv). The resulting mixture was stirred at 85° C. overnight under a nitrogen atmosphere. The resulting mixture was then concentrated under reduced pressure. The residue was purified by silica gel column chromatography eluted with PE:EtOAc (3:1) to afford tert-butyl 4-(4-bromo-2,3-difluorophenyl)piperazine-1-carboxylate (8 g, 72.07%) as an orange solid. LCMS: C₁₅H₁₉BrF₂N₂O₂ requires: 376.1, found: m/z=377.0 [M+H]⁺.

### Step-2: Synthesis of tert-butyl 4-[2,3-difluoro-4-(4,4,5,5-tetramethyl-1,3,2-dioxaborolan-2-yl)phenyl]piperazine-1-carboxylate (3)

To a mixture of tert-butyl 4-(4-bromo-2,3-difluorophenyl)piperazine-1-carboxylate (6 g, 15.905 mmol, 1 equiv) and 4,4,5,5-tetramethyl-2-(tetramethyl-1,3,2-dioxaborolan-2-yl)-1,3,2-dioxaborolane (B₂Pin₂) (8.08 g, 31.810 mmol, 2 equiv) in 1,4-dioxane (100 mL) was added AcOK (3.12 g, 31.810 mmol, 2 equiv) and Pd(dppf)Cl₂ (1.3 g, 1.591 mmol, 0.1 equiv). The resulting mixture was stirred at 90° C. overnight under a nitrogen atmosphere. The resulting mixture was then concentrated under reduced pressure. The residue was purified by silica gel column chromatography eluted with PE:EtOAc (4:1) to afford tert-butyl 4-[2,3-difluoro-4-(4,4,5,5-tetramethyl-1,3,2-dioxaborolan-2-yl)phenyl]piperazine-1-carboxylate (5.6 g, 82.98%) as an orange solid. LCMS: C₂₁H₃₁BF₂N₂O₄ requires: 424.2, found: m/z=425.3 [M+H]⁺.

### Step-3: Synthesis of tert-butyl 4-{4-[4-bromo-3-(pyridin-4-yl)pyrazol-1-yl]-2,3-difluorophenyl}piperazine-1-carboxylate (4)

To a mixture of tert-butyl 4-[2,3-difluoro-4-(4,4,5,5-tetramethyl-1,3,2-dioxaborolan-2-yl)phenyl]piperazine-1-carboxylate (5.6 g, 13.198 mmol, 1 equiv) and 4-(4-bromo-1H-pyrazol-3-yl)pyridine (3.25 g, 14.518 mmol, 1.1 equiv) in pyridine (50 mL) was added Cu(OAc)₂ (4.79 g, 26.396 mmol, 2 equiv) and molecular sieves (4A) (5.6 g). The resulting mixture was stirred at 60° C. overnight under an oxygen atmosphere. The resulting mixture was then extracted with EtOAc. The combined organic layers were washed with water and dried over anhydrous Na₂SO₄. After filtration, the filtrate was concentrated under reduced pressure. The residue was purified by silica gel column chromatography eluted with PE:EtOAc (1:1) to afford tert-butyl 4-{4-[4-bromo-3-(pyridin-4-yl)pyrazol-1-yl]-2,3-difluorophenyl}piperazine-1-carboxylate (700 mg, 10.19%) as a white solid. LCMS: C₂₃H₂₄BrF₂N₅O₂ requires: 519.1, found: m/z=520.1 [M+H]⁺.

### Step-4: Synthesis of tert-butyl 4-[4-(4-{2,5-difluoro-3-[(pyrrolidine-1-sulfonyl)amino]phenyl}-3-(pyridin-4-yl)pyrazol-1-yl)-2,3-difluorophenyl]piperazine-1-carboxylate (5)

To a mixture of tert-butyl 4-{4-[4-bromo-3-(pyridin-4-yl)pyrazol-1-yl]-2,3-difluorophenyl}piperazine-1-carboxylate (600 mg, 1.153 mmol, 1 equiv) and N-[2,5-difluoro-3-(4,4,5,5-tetramethyl-1,3,2-dioxaborolan-2-yl)phenyl]pyrrolidine-1-sulfonamide (447 mg, 1.153 mmol, 1 equiv) in dioxane (5 mL) was added Pd(AMPHOS)₂Cl₂ (81 mg, 0.115 mmol, 0.1 equiv) and CsF (350 mg, 2.306 mmol, 2 equiv) in H₂O (1 mL). The resulting mixture was stirred at 90° C. for one hour under a nitrogen atmosphere. The resulting mixture was then concentrated under reduced pressure. The residue was purified by silica gel column chromatography eluted with PE:EtOAc (2:3). The residue was then purified by trituration with EtOAc. This resulted in tert-butyl 4-[4-(4-{2,5-difluoro-3-[(pyrrolidine-1-sulfonyl)amino]phenyl}-3-(pyridin-4-yl)pyrazol-1-yl)-2,3-difluorophenyl]piperazine-1-carboxylate (400 mg, 49.44%) as a white solid. LCMS: C₃₃H₃₅F₄N₇O₄S requires: 701.2, found: m/z=702.3 [M+H]⁺.

### Step-5: Synthesis of N-(3-{1-[2,3-difluoro-4-(piperazin-1-yl)phenyl]-3-(pyridin-4-yl)pyrazol-4-yl}-2,5-difluorophenyl)pyrrolidine-1-sulfonamide Trifluoroacetic Acid Salt (6)

A mixture of tert-butyl 4-[4-(4-{2,5-difluoro-3-[(pyrrolidine-1-sulfonyl)amino]phenyl}-3-(pyridin-4-yl)pyrazol-1-yl)-2,3-difluorophenyl]piperazine-1-carboxylate (400 mg, 0.570 mmol, 1 equiv) in TFA (4 mL) was stirred at 0° C. for hone hour. The resulting mixture was then concentrated under vacuum and lyophilized. This resulted in N-(3-{1-[2,3-difluoro-4-(piperazin-1-yl)phenyl]-3-(pyridin-4-yl)pyrazol-4-yl}-2,5-difluorophenyl)pyrrolidine-1-sulfonamide trifluoroacetic acid salt (376.6 mg, crude) as a yellow solid. LCMS: C₂₈H₂₇F₄N₇O₂S requires: 601.2, found: m/z=602.3 [M+H]⁺. ¹H NMR (400 MHz, Methanol-d₄) δ 8.89-8.77 (m, 2H), 8.51-8.46 (m, 1H), 8.21-8.14 (m, 2H), 7.79-7.70 (m, 1H), 7.47-7.37 (m, 1H), 7.19-7.05 (m, 2H), 3.52-3.43 (m, 8H), 3.37-3.35 (m, 1H), 3.34-3.33 (m, 3H), 1.98-1.86 (m, 4H).

### Synthesis of N-[2,5-difluoro-3-(1-{4-[(3R)-3-methylpiperazin-1-yl]phenyl}-3-(pyridin-4-yl)pyrazol-4-yl)phenyl]pyrrolidine-1-sulfonamide Trifluoroacetic Acid Salt

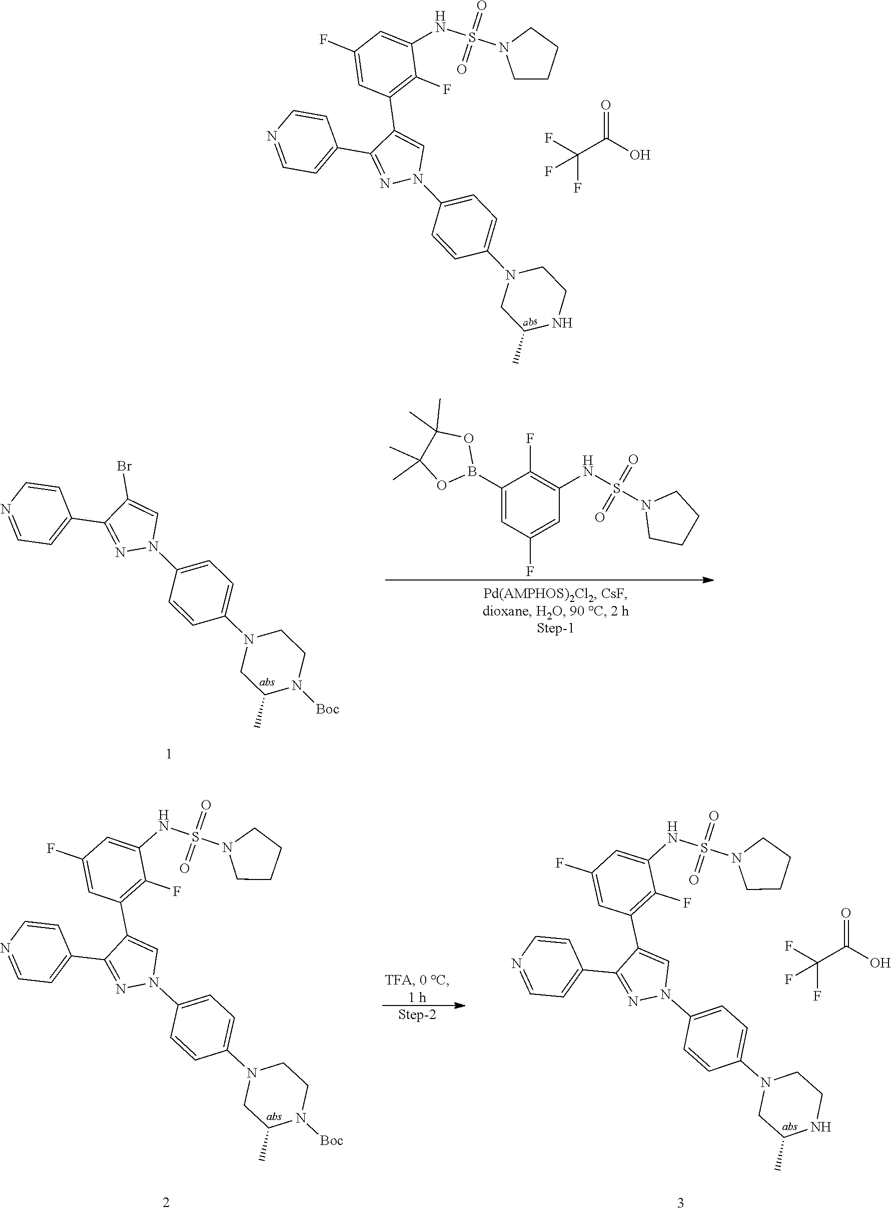

### Step-1: Synthesis of tert-butyl (2R)-4-[4-(4-{2,5-difluoro-3-[(pyrrolidine-1-sulfonyl)amino]phenyl}-3-(pyridin-4-yl)pyrazol-1-yl)phenyl]-2-methylpiperazine-1-carboxylate (2)

To a mixture of tert-butyl (2R)-4-{4-[4-bromo-3-(pyridin-4-yl)pyrazol-1-yl]phenyl}-2-methylpiperazine-1-carboxylate (80 mg, 0.161 mmol, 1 equiv) and N-[2,5-difluoro-3-(4,4,5,5-tetramethyl-1,3,2-dioxaborolan-2-yl)phenyl]pyrrolidine-1-sulfonamide (62 mg, 0.161 mmol, 1 equiv) in dioxane (1 mL) was added Pd(AMPHOS)₂Cl₂ (11 mg, 0.016 mmol, 0.1 equiv) and CsF (49 mg, 0.322 mmol, 2 equiv) in H₂O (0.2 mL). The resulting mixture was stirred at 90° C. for 2 h under a nitrogen atmosphere. The resulting mixture was then concentrated under vacuum. The residue was purified by silica gel column chromatography eluted with PE:EtOAc (2:3). The residue was then purified by trituration with EtOAc. This resulted in tert-butyl (2R)-4-[4-(4-{2,5-difluoro-3-[(pyrrolidine-1-sulfonyl)amino]phenyl}-3-(pyridin-4-yl)pyrazol-1-yl)phenyl]-2-methylpiperazine-1-carboxylate (20 mg, 17.41%) as an off-white solid. LCMS: C₃₄H₃₉F₂N₇O₄S requires: 679.2, found: m/z=680.4 [M+H]⁺.

### Step-2: Synthesis of N-[2,5-difluoro-3-(1-{4-[(3R)-3-methylpiperazin-1-yl]phenyl}-3-(pyridin-4-yl)pyrazol-4-yl)phenyl]pyrrolidine-1-sulfonamide Trifluoroacetic Acid Salt (3)

A mixture of tert-butyl (2R)-4-[4-(4-{2,5-difluoro-3-[(pyrrolidine-1-sulfonyl)amino]phenyl}-3-(pyridin-4-yl)pyrazol-1-yl)phenyl]-2-methylpiperazine-1-carboxylate (20 mg, 0.029 mmol, 1 equiv) in TFA (1 mL) was stirred at 0° C. for one hour. The resulting mixture was then concentrated under vacuum and lyophilized. This resulted in N-[2,5-difluoro-3-(1-{4-[(3R)-3-methylpiperazin-1-yl]phenyl}-3-(pyridin-4-yl)pyrazol-4-yl)phenyl]pyrrolidine-1-sulfonamide trifluoroacetic acid salt (11 mg, crude) as a yellow solid. LCMS: C₂₉H₃₁F₂N₇O₂S requires: 579.2, found: m/z=580.3 [M+H]⁺. ¹H NMR (400 MHz, Methanol-d₄) δ 8.81-8.75 (m, 2H), 8.62-8.58 (m, 1H), 8.23-8.16 (m, 2H), 7.94-7.86 (m, 2H), 7.46-7.37 (m, 1H), 7.29-7.20 (m, 2H), 7.14-7.06 (m, 1H), 4.00-3.86 (m, 2H), 3.63-3.50 (m, 2H), 3.41-3.29 (m, 5H), 3.19-3.07 (m, 1H), 2.96-2.85 (m, 1H), 1.98-1.85 (m, 4H), 1.48-1.42 (m, 3H).

### Synthesis of N-[2,5-difluoro-3-(1-{2-fluoro-4-[(3R)-3-methylpiperazin-1-yl]phenyl}-3-(pyridin-4-yl)pyrazol-4-yl)phenyl]pyrrolidine-1-sulfonamide Trifluoroacetic Acid Salt (6)

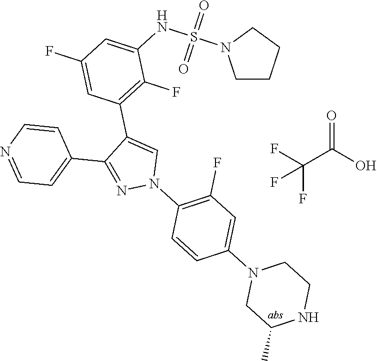 [SMILES: C[C@@H]1CN(c2ccc(-n3cc(-c4cc(F)cc(NS(=O)(=O)N5CCCC5)c4F)c(-c4ccncc4)n3)c(F)c2)CCN1 ; O=C(O)C(F)(F)F]

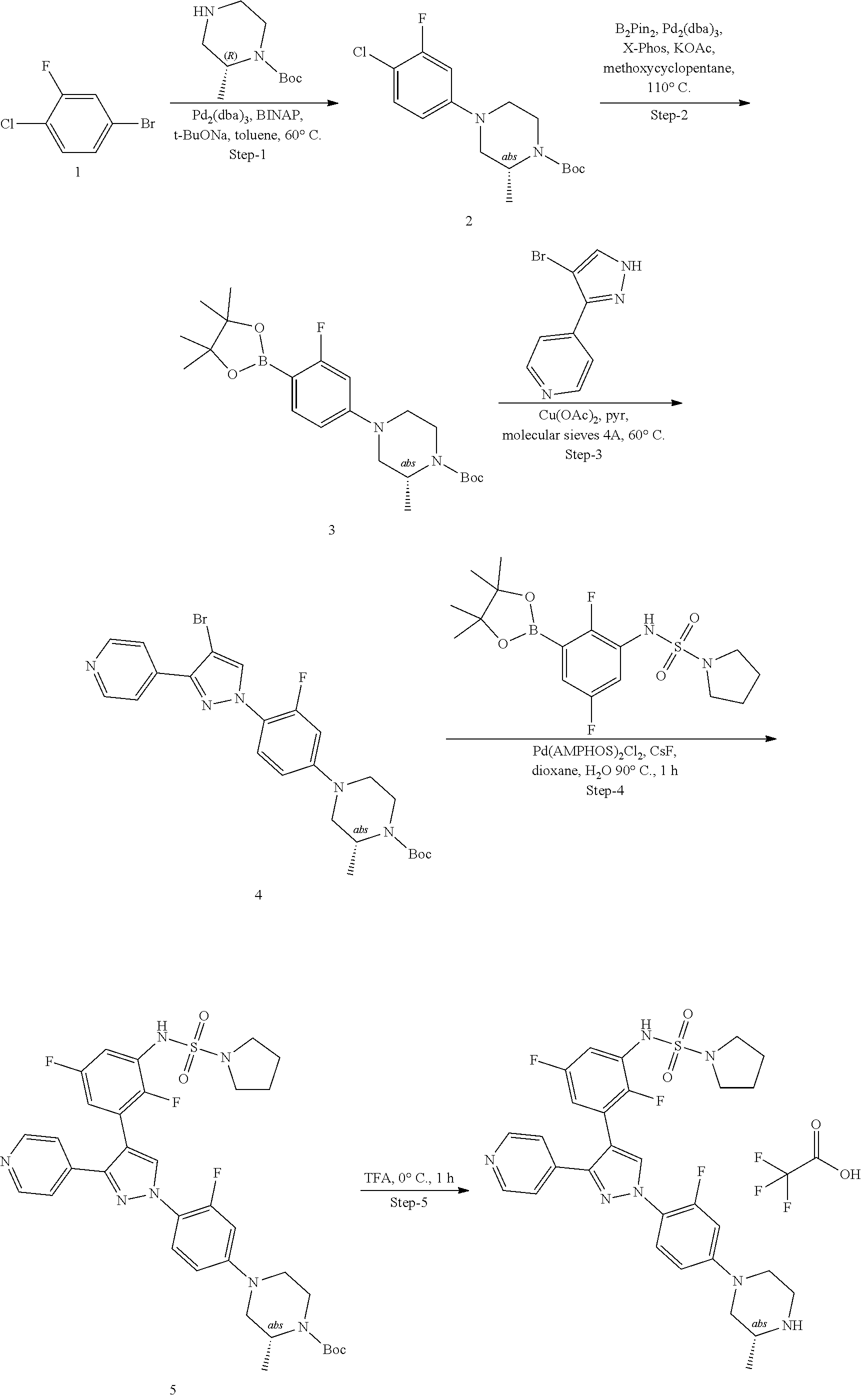

### Step-1: Synthesis of tert-butyl (2R)-4-(4-chloro-3-fluorophenyl)-2-methylpiperazine-1-carboxylate (2)

To a mixture of 4-bromo-1-chloro-2-fluorobenzene (8 g, 38.197 mmol, 1 equiv) and tert-butyl (2R)-2-methylpiperazine-1-carboxylate (7.65 g, 38.196 mmol, 1 equiv) in toluene (80 mL) was added BINAP (0.48 g, 0.764 mmol, 0.02 equiv), t-BuONa (5.14 g, 53.476 mmol, 1.4 equiv), and Pd₂(dba)₃ (0.35 g, 0.382 mmol, 0.01 equiv). The resulting mixture was stirred at 60° C. overnight under a nitrogen atmosphere. The resulting mixture was then concentrated under reduced pressure. The residue was purified by silica gel column chromatography eluted with PE:EtOAc (1:1) to afford tert-butyl (2R)-4-(4-chloro-3-fluorophenyl)-2-methylpiperazine-1-carboxylate (10 g, 79.62%) as a white solid. LCMS: C₁₆H₂₂ClFN₂O₂ requires: 328.1, found: m/z=329.0 [M+H]⁺.

### Step-2: Synthesis of tert-butyl (2R)-4-[3-fluoro-4-(4,4,5,5-tetramethyl-1,3,2-dioxaborolan-2-yl)phenyl]-2-methylpiperazine-1-carboxylate (3)

To a mixture of tert-butyl (2R)-4-(4-chloro-3-fluorophenyl)-2-methylpiperazine-1-carboxylate (10 g, 30.413 mmol, 1 equiv) and 4,4,5,5-tetramethyl-2-(4,4,5,5-tetramethyl-1,3,2-dioxaborolan-2-yl)-1,3,2-dioxaborolane (B₂Pin₂) (11.58 g, 45.620 mmol, 1.5 equiv) in methoxycyclopentane (100 mL) was added XPhos (0.87 g, 1.825 mmol, 0.06 equiv), AcOK (8.95 g, 91.239 mmol, 3 equiv), and Pd₂(dba)₃ (0.84 g, 0.912 mmol, 0.03 equiv). The resulting mixture was stirred at 110° C. overnight under a nitrogen atmosphere. The resulting mixture was then concentrated under reduced pressure. The residue was purified by silica gel column chromatography eluted with PE:EtOAc (9:1) to afford tert-butyl (2R)-4-[3-fluoro-4-(4,4,5,5-tetramethyl-1,3,2-dioxaborolan-2-yl)phenyl]-2-methylpiperazine-1-carboxylate (12 g, 93.87%) as a brown oil. LCMS: C₂₂H₃₄BFN₂O₄ requires: 420.3, found: m/z=421.1 [M+H]⁺.

### Step-3: Synthesis of tert-butyl (2R)-4-{4-[4-bromo-3-(pyridin-4-yl)pyrazol-1-yl]-3-fluorophenyl}-2-methylpiperazine-1-carboxylate (4)

To a mixture of tert-butyl (2R)-4-[3-fluoro-4-(4,4,5,5-tetramethyl-1,3,2-dioxaborolan-2-yl)phenyl]-2-methylpiperazine-1-carboxylate (5 g, 11.895 mmol, 1 equiv) and 4-(4-bromo-1H-pyrazol-3-yl)pyridine (2.93 g, 13.085 mmol, 1.1 equiv) in pyridine (50 mL) was added Cu(OAc)₂ (4.32 g, 23.790 mmol, 2 equiv) and molecular sieves (4A) (5 g). The resulting mixture was stirred at 60° C. overnight under an oxygen atmosphere. The resulting mixture was then extracted with EtOAc. The combined organic layers were washed with water and dried over anhydrous Na₂SO₄. After filtration, the filtrate was concentrated under reduced pressure. The residue was purified by silica gel column chromatography eluted with PE:EtOAc (1:1) to afford tert-butyl (2R)-4-{4-[4-bromo-3-(pyridin-4-yl)pyrazol-1-yl]-3-fluorophenyl}-2-methylpiperazine-1-carboxylate (2.1 g, 34.19%) as a white solid. LCMS: C₂₄H₂₇BrFN₅O₂ requires: 515.1, found: m/z=516.1 [M+H]⁺.

### Step-4: Synthesis of tert-butyl (2R)-4-[4-(4-{2,5-difluoro-3-[(pyrrolidine-1-sulfonyl)amino]phenyl}-3-(pyridin-4-yl)pyrazol-1-yl)-3-fluorophenyl]-2-methylpiperazine-1-carboxylate (5)

To a mixture of tert-butyl (2R)-4-{4-[4-bromo-3-(pyridin-4-yl)pyrazol-1-yl]-3-fluorophenyl}-2-methylpiperazine-1-carboxylate (55 mg, 0.107 mmol, 1 equiv) and N-[2,5-difluoro-3-(4,4,5,5-tetramethyl-1,3,2-dioxaborolan-2-yl)phenyl]pyrrolidine-1-sulfonamide (41 mg, 0.107 mmol, 1 equiv) in dioxane (1 mL) was added CsF (32 mg, 0.214 mmol, 2 equiv) and Pd(AMPHOS)₂Cl₂ (8 mg, 0.011 mmol, 0.1 equiv) in H₂O (0.2 mL). The resulting mixture was stirred at 90° C. for one hour under a nitrogen atmosphere. The resulting mixture was then concentrated under reduced pressure. The residue was purified by silica gel column chromatography eluted with PE:EtOAc (2:3) to afford tert-butyl (2R)-4-[4-(4-{2,5-difluoro-3-[(pyrrolidine-1-sulfonyl)amino]phenyl}-3-(pyridin-4-yl)pyrazol-1-yl)-3-fluorophenyl]-2-methylpiperazine-1-carboxylate (20 mg, 26.91%) as a yellow oil. LCMS: C₃₄H₃₈F₃N₇O₄S requires: 697.3, found: m/z=698.4 [M+H]⁺.

### Step-5: Synthesis of N-[2,5-difluoro-3-(1-{2-fluoro-4-[(3R)-3-methylpiperazin-1-yl]phenyl}-3-(pyridin-4-yl)pyrazol-4-yl)phenyl]pyrrolidine-1-sulfonamide Trifluoroacetic Acid Salt (6)

A mixture of tert-butyl (2R)-4-[4-(4-{2,5-difluoro-3-[(pyrrolidine-1-sulfonyl)amino]phenyl}-3-(pyridin-4-yl)pyrazol-1-yl)-3-fluorophenyl]-2-methylpiperazine-1-carboxylate (20 mg, 0.029 mmol, 1 equiv) in TFA (1 mL) was stirred at 0° C. for one hour. The resulting mixture was then concentrated under vacuum and lyophilized. This resulted in N-[2,5-difluoro-3-(1-{2-fluoro-4-[(3R)-3-methylpiperazin-1-yl]phenyl}-3-(pyridin-4-yl)pyrazol-4-yl)phenyl]pyrrolidine-1-sulfonamide trifluoroacetic acid salt (14 mg, crude) as a yellow solid. LCMS: C₂₉H₃₀F₃N₇O₂S requires: 597.2, found: m/z=598.3 [M+H]⁺. ¹H NMR (400 MHz, Methanol-d₄) δ 8.83-8.72 (m, 2H), 8.43-8.34 (m, 1H), 8.19-8.09 (m, 2H), 7.93-7.78 (m, 1H), 7.49-7.38 (m, 1H), 7.19-7.00 (m, 3H), 4.09-3.90 (m, 2H), 3.64-3.49 (m, 2H), 3.40-3.34 (m, 3H), 3.32-3.29 (m, 2H), 3.25-3.11 (m, 1H), 3.03-2.87 (m, 1H), 2.00-1.84 (m, 4H), 1.53-1.39 (m, 3H).

### Synthesis of (1R,4r)-4-((6-((RS)-2,6-dioxopiperidin-3-yl)-3,4-dihydroisoquinolin-2(1H)-yl)methyl)cyclohexane-1-carboxylic Acid

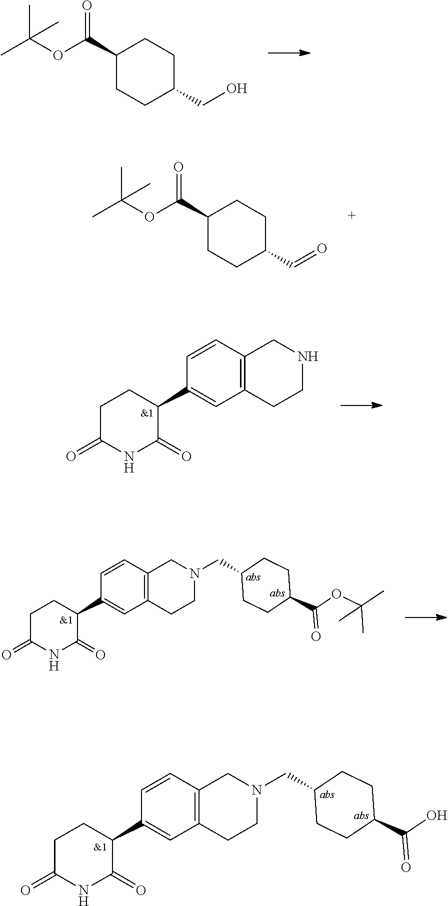 [SMILES: CC(C)(C)OC(=O)[C@H]1CC[C@H](CO)CC1 ; CC(C)(C)OC(=O)[C@H]1CC[C@H](C=O)CC1 ; O=C1CC[C@@H](c2ccc3c(c2)CCNC3)C(=O)N1 ; CC(C)(C)OC(=O)[C@H]1CC[C@H](CN2CCc3cc([C@@H]4CCC(=O)NC4=O)ccc3C2)CC1 ; O=C1CC[C@@H](c2ccc3c(c2)CCN(C[C@H]2CC[C@H](C(=O)O)CC2)C3)C(=O)N1]

### Step-1: Synthesis of tert-butyl (1r,4r)-4-formylcyclohexane-1-carboxylate

To a solution of tert-butyl (1r,4r)-4-(hydroxymethyl)cyclohexane-1-carboxylate (300.00 mg, 1.3999 mmol) in DMSO (1 mL) was added triethylamine (1.97 mL, 1416.57 mg, 13.9987 mmol) followed by the addition of sulfur trioxide pyridine complex (1.11 g, 6.9994 mmol) in DMSO (2 mL). The reaction mixture was stirred for 16 h. Additional sulfur trioxide pyridine complex (0.56 g, 3.5 mmol) in DMSO (0.5 mL) and triethylamine (0.985 mL, 7 mmol) was added after 16 h. The reaction was complete as indicated by TLC. i-PrOH (0.5 mL) was added to the reaction mixture. After stirring for three hours, the reaction mixture was concentrated under reduced pressure. The crude material was used in the next step without purification.

### Step-2: tert-butyl (1R,4r)-4-((6-((RS)-2,6-dioxopiperidin-3-yl)-3,4-dihydroisoquinolin-2(1H)-yl)methyl)cyclohexane-1-carboxylate

To a solution of rac-(3R)-3-(1,2,3,4-tetrahydroisoquinolin-6-yl)piperidine-2,6-dione (100.00 mg, 0.4093 mmol) and tert-butyl (1r,4r)-4-formylcyclohexane-1-carboxylate (86.90 mg, 0.4093 mmol) in 2 ml DMSO (2 mL) was added triethylamine (57.5 μL, 0.41 mmol) and sodium triacetoxyborohydride (0.26 g, 1.2280 mmol) and the reaction mixture was stirred for thirty minutes. EtOAc (30 mL) was added and the organic solution was washed with water and brine, dried over sodium sulfate, filtered, and concentrated. The crude product was loaded onto a Redi-Sep prepacked silica gel column eluting with MeOH in DCM (0-10%), to provide tert-butyl (1R,4r)-4-((6-((RS)-2,6-dioxopiperidin-3-yl)-3,4-dihydroisoquinolin-2(1H)-yl)methyl)cyclohexane-1-carboxylate (0.18 g, 99.81%). LCMS C₂₆H₃₆N₂O₄ requires: 440.3, found: m/z=441.7 [M+H]⁺.

### Step-3: (1R,4r)-4-((6-((RS)-2,6-dioxopiperidin-3-yl)-3,4-dihydroisoquinolin-2(1H)-yl)methyl)cyclohexane-1-carboxylic Acid

To a solution of tert-butyl (1R,4r)-4-((6-((RS)-2,6-dioxopiperidin-3-yl)-3,4-dihydroisoquinolin-2(1H)-yl)methyl)cyclohexane-1-carboxylate (180.00 mg, 0.4085 mmol) in DCM (3 mL) was added trifluoroacetic acid (1.5 mL) and the reaction was stirred for one hour. TFA and DCM were evaporated under reduced pressure and the crude product was pumped to dryness to afford (1R,4r)-4-((6-((RS)-2,6-dioxopiperidin-3-yl)-3,4-dihydroisoquinolin-2(1H)-yl)methyl)cyclohexane-1-carboxylic acid in quantitative yield. LCMS C₂₂H₂₈N₂O₄ requires: 384.2, found m/z=385.6 [M+H]⁺.

### Synthesis of rac-(R)-1-(1-(5-(2,6-dioxopiperidin-3-yl)pyridin-2-yl)piperidine-4-carbonyl)-4-methylpiperidine-4-carboxylic Acid

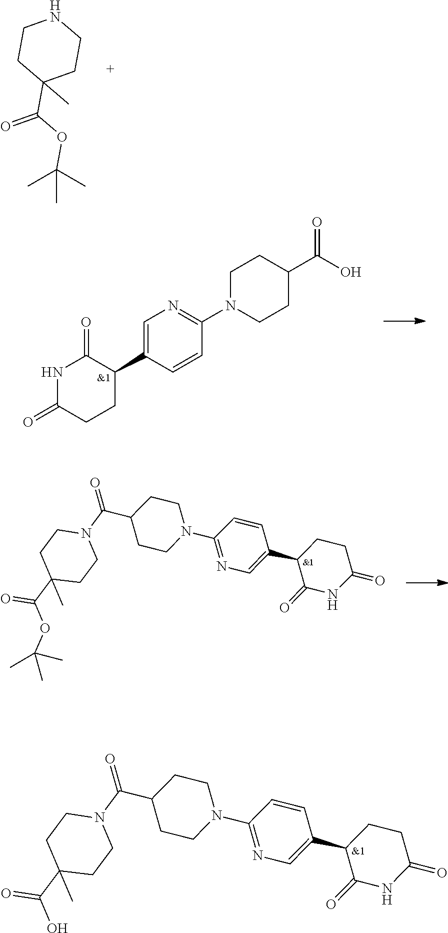 [SMILES: CC(C)(C)OC(=O)C1(C)CCNCC1 ; O=C1CC[C@H](c2ccc(N3CCC(C(=O)O)CC3)nc2)C(=O)N1 ; CC(C)(C)OC(=O)C1(C)CCN(C(=O)C2CCN(c3ccc([C@H]4CCC(=O)NC4=O)cn3)CC2)CC1 ; CC1(C(=O)O)CCN(C(=O)C2CCN(c3ccc([C@H]4CCC(=O)NC4=O)cn3)CC2)CC1]

### Step-1: rac-tert-butyl (R)-1-(1-(5-(2,6-dioxopiperidin-3-yl)pyridin-2-yl)piperidine-4-carbonyl)-4-methylpiperidine-4-carboxylate

To a solution of rac-(R)-1-(5-(2,6-dioxopiperidin-3-yl)pyridin-2-yl)piperidine-4-carboxylic acid (214.00 mg, 0.6743 mmol) and [(dimethylamino)({[1,2,3]triazolo[4,5-b]pyridin-3-yloxy})methylidene]dimethylazanium; hexafluoro-lambda5-phosphanide (282.05 mg, 0.7418 mmol) in DMF (2 mL) was added N,N-diisopropylethylamine (435.79 mg, 3.3717 mmol). Then tert-butyl 4-methylpiperidine-4-carboxylate (147.83 mg, 0.7418 mmol) in DMF (1 mL) was added. After stirring for one hour, the crude product was dissolved in ethyl acetate (40 mL) and the solution was then washed with water and brine, dried over sodium sulfate, filtered, and concentrated. The crude product was loaded onto a Redi-Sep prepacked silica gel column eluting with MeOH in DCM (0-6%) to afford rac-tert-butyl (R)-1-(1-(5-(2,6-dioxopiperidin-3-yl)pyridin-2-yl)piperidine-4-carbonyl)-4-methylpiperidine-4-carboxylate (0.236 g, 70.19%). LCMS: C₂₇H₃₈N₄O₅ requires: 498.6, found: m/z=499.6 [M+H]⁺.

### Step-2: rac-(R)-1-(1-(5-(2,6-dioxopiperidin-3-yl)pyridin-2-yl)piperidine-4-carbonyl)-4-methylpiperidine-4-carboxylic Acid

rac-tert-butyl (R)-1-(1-(5-(2,6-dioxopiperidin-3-yl)pyridin-2-yl)piperidine-4-carbonyl)-4-methylpiperidine-4-carboxylate (0.236 g, 0.4733 mmol) was dissolved in DCM (4 mL) and then trifluoroacetic acid (2 mL) was added. The solution was stirred for three hours. TFA and DCM were evaporated under reduced pressure. The crude product was loaded onto a Redi-Sep prepacked HP C18 (30 grams) column eluting with acetonitrile in water (5%-80%) to afford rac-(R)-1-(1-(5-(2,6-dioxopiperidin-3-yl)pyridin-2-yl)piperidine-4-carbonyl)-4-methylpiperidine-4-carboxylic acid (0.132 g, 44.23%). LCMS: C₂₃H₃₀N₄O₅ required: 442.5, found: m/z=443.6 [M+H]⁺.

### Synthesis of rac-(R)-1-(2-(1-(5-(2,6-dioxopiperidin-3-yl)pyridin-2-yl)piperidin-4-yl)acetyl)-4-methylpiperidine-4-carboxylic Acid

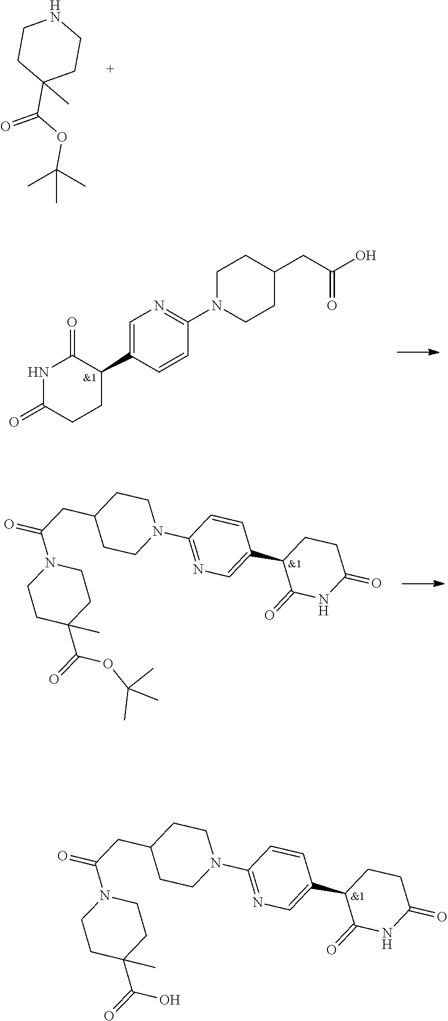 [SMILES: CC(C)(C)OC(=O)C1(C)CCNCC1 ; O=C(O)CC1CCN(c2ccc([C@H]3CCC(=O)NC3=O)cn2)CC1 ; CC(C)(C)OC(=O)C1(C)CCN(C(=O)CC2CCN(c3ccc([C@H]4CCC(=O)NC4=O)cn3)CC2)CC1 ; CC1(C(=O)O)CCN(C(=O)CC2CCN(c3ccc([C@H]4CCC(=O)NC4=O)cn3)CC2)CC1]

### Step-1: rac-tert-butyl (R)-1-(2-(1-(5-(2,6-dioxopiperidin-3-yl)pyridin-2-yl)piperidin-4-yl)acetyl)-4-methylpiperidine-4-carboxylate

To a solution of rac-(R)-2-(1-(5-(2,6-dioxopiperidin-3-yl)pyridin-2-yl)piperidin-4-yl)acetic acid (200.90 mg, 0.6063 mmol) and [(dimethylamino)({[1,2,3]triazolo[4,5-b]pyridine-3-yloxy})methylidene]dimethylazanium; hexafluoro-lambda5-phosphanide (253.58 mg, 0.6669 mmol) in DMF (2 mL) was added N,N-diisopropylethylamine (391.79 mg, 3.0313 mmol). Then tert-butyl 4-methylpiperidine-4-carboxylate (122.03 mg, 0.6123 mmol) in DMF (1 mL) was added. After stirring for one hour, the crude product was dissolved in ethyl acetate, washed with water and brine, dried over Na₂SO₄, filtered, and concentrated. The crude product was loaded onto a Redi-Sep prepacked silica gel column eluting with MeOH in DCM (0-6%) to afford the desired product which was contaminated with tert-butyl 4-methylpiperidine-4-carboxylate. The crude product was dissolved in DCM, washed with NH₄Cl solution three times, dried over Na₂SO₄, filtered, and concentrated. The product was used in the next step without purification. rac-tert-butyl (R)-1-(2-(1-(5-(2,6-dioxopiperidin-3-yl)pyridin-2-yl)piperidin-4-yl)acetyl)-4-methylpiperidine-4-carboxylate (0.26 g, 83.65%). LCMS: C₂₈H₄₀N₄O₅ requires: 512.3, found: m/z=513.3 [M+H]⁺.

### Step-2: rac-(R)-1-(2-(1-(5-(2,6-dioxopiperidin-3-yl)pyridin-2-yl)piperidin-4-yl)acetyl)-4-methylpiperidine-4-carboxylic Acid

rac-tert-butyl (R)-1-(2-(1-(5-(2,6-dioxopiperidin-3-yl)pyridin-2-yl)piperidin-4-yl)acetyl)-4-methylpiperidine-4-carboxylate (260.00 mg, 0.5072 mmol) was dissolved in DCM (3 mL) and trifluoroacetic acid (2 mL) was added. The solution was then stirred for three hours. TFA and DCM were evaporated under reduced pressure. The crude product was loaded onto a Redi-Sep prepacked HP C18 (30 grams) column eluting with acetonitrile in water (0%-60%) to afford rac-(R)-1-(2-(1-(5-(2,6-dioxopiperidin-3-yl)pyridin-2-yl)piperidin-4-yl)acetyl)-4-methylpiperidine-4-carboxylic acid (0.19 g, 82.06%) as white solid. LCMS: C₂₄H₃₂N₄O₅ required: 456.2, found: m/z=457.3 [M+H]⁺.

### Synthesis of rac-(R)-1-(5-(2,6-dioxopiperidin-3-yl)pyridin-2-yl)-4-methylpiperidine-4-carboxylic Acid

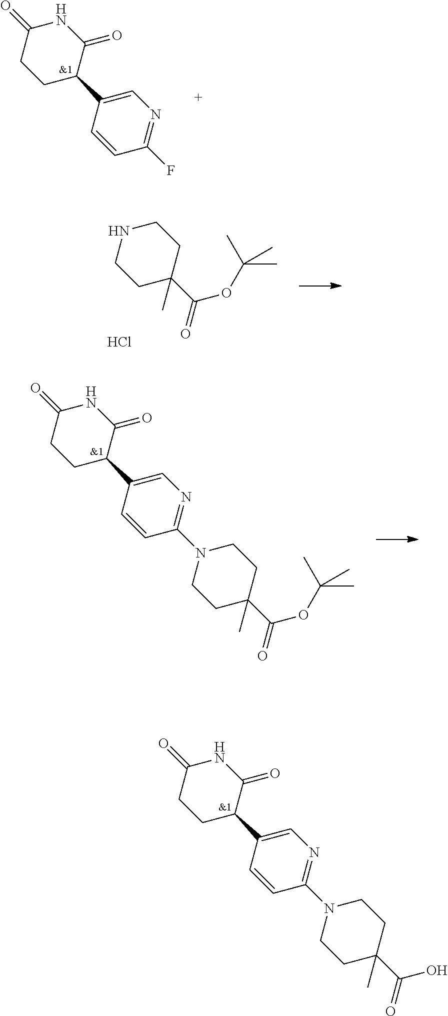 [SMILES: O=C1CC[C@H](c2ccc(F)nc2)C(=O)N1 ; CC(C)(C)OC(=O)C1(C)CCNCC1 ; Cl ; CC(C)(C)OC(=O)C1(C)CCN(c2ccc([C@H]3CCC(=O)NC3=O)cn2)CC1 ; CC1(C(=O)O)CCN(c2ccc([C@H]3CCC(=O)NC3=O)cn2)CC1]

### Step-1: rac-tert-butyl (R)-1-(5-(2,6-dioxopiperidin-3-yl)pyridin-2-yl)-4-methylpiperidine-4-carboxylate

A mixture of rac-(R)-3-(6-fluoropyridin-3-yl)piperidine-2,6-dione (200.00 mg, 0.9607 mmol), tert-butyl 4-methylpiperidine-4-carboxylate hydrochloride (452.95 mg, 1.9213 mmol), and N,N-diisopropylethylamine (671.14 μL, 496.65 mg, 3.8426 mmol) in DMSO (2.00 mL) was heated at 120° C. for sixteen hours. The crude product was then cooled down and water (5 mL) was added. The solution was then extracted with EtOAc twice. The organic solution was dried over sodium sulfate, filtered, and concentrated. The crude product was loaded onto a Redi-Sep prepacked silica gel column eluting with EtOAc:heptane (10-100%) to provide rac-tert-butyl (R)-1-(5-(2,6-dioxopiperidin-3-yl)pyridin-2-yl)-4-methylpiperidine-4-carboxylate (0.3 g, 80.59%). LCMS: C₂₁H₂₉N₃O₄ required: 387.2, found: m/z=388.5 [M+H]⁺.

### Step-2: rac-(R)-1-(5-(2,6-dioxopiperidin-3-yl)pyridin-2-yl)-4-methylpiperidine-4-carboxylic Acid

rac-tert-butyl (R)-1-(5-(2,6-dioxopiperidin-3-yl)pyridin-2-yl)-4-methylpiperidine-4-carboxylate (300.00 mg, 0.7742 mmol) was dissolved in DCM (2 mL) and then TFA (2 mL) was added. The reaction was then stirred for three hours. TFA and DCM were evaporated. The crude product was loaded onto a Redi-Sep prepacked HP C18 (30 grams) column eluting with acetonitrile:water (0-90%) to afford rac-(R)-1-(5-(2,6-dioxopiperidin-3-yl)pyridin-2-yl)-4-methylpiperidine-4-carboxylic acid (0.21 g, 81.85%) as white solid. LCMS C₁₇H₂₁N₃O₄ requires: 331.2, found: m/z=332.4 [M+H]⁺.

### Synthesis of rac-(R)-2-(5-(2,6-dioxopiperidin-3-yl)pyridin-2-yl)-2-azaspiro[3.3]heptane-6-carbaldehyde

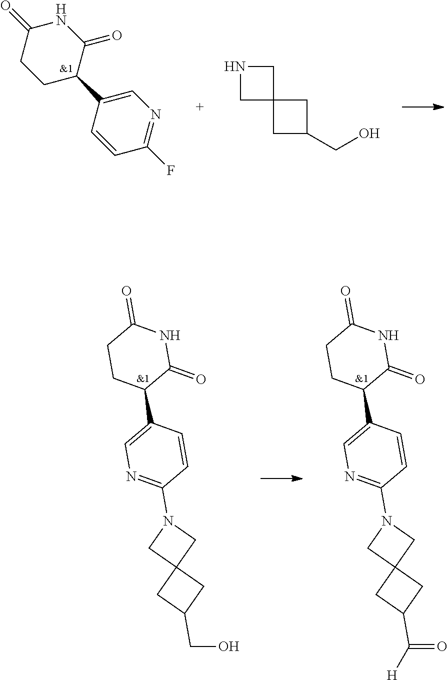 [SMILES: O=C1CC[C@H](c2ccc(F)nc2)C(=O)N1 ; OCC1CC2(CNC2)C1 ; O=C1CC[C@H](c2ccc(N3CC4(CC(CO)C4)C3)nc2)C(=O)N1 ; O=CC1CC2(C1)CN(c1ccc([C@H]3CCC(=O)NC3=O)cn1)C2]

### Step-1: rac-(R)-3-(6-(6-(hydroxymethyl)-2-azaspiro[3.3]heptan-2-yl)pyridin-3-yl)piperidine-2,6-dione

A mixture of rac-(R)-3-(6-fluoropyridin-3-yl)piperidine-2,6-dione (250.00 mg, 1.2008 mmol), (2-azaspiro[3.3]heptan-6-yl)methanol (413.57 mg, 1.2008 mmol), and N,N-diisopropylethylamine (1.05 mL, 776.01 mg, 6.0041 mmol) in DMSO (2.00 mL) was heated at 120° C. for sixteen hours. The reaction mixture was lyophilized to remove DMSO. The crude product was loaded onto a Redi-Sep prepacked silica gel column eluting with MeOH in DCM (3-20%) to provide rac-(R)-3-(6-(6-(hydroxymethyl)-2-azaspiro[3.3]heptan-2-yl)pyridin-3-yl)piperidine-2,6-dione (0.12 g, 31.69%). LCMS: C₁₇H₂₁N₃O₃ required: 315.2, found: m/z=316.5 [M+H]⁺. ¹H NMR (500 MHz, CDCl₃) δ 8.14-7.86 (m, 2H), 6.30 (d, J=8.6 Hz, 1H), 4.06 (d, J=9.8 Hz, 2H), 3.96 (d, J=7.4 Hz, 2H), 3.65 (dq, J=15.8, 8.8, 7.0 Hz, 4H), 3.52 (s, 2H), 2.80 (dt, J=17.7, 4.8 Hz, 1H), 2.74-2.58 (m, 2H), 2.52-2.12 (m, 7H), 2.11-1.99 (m, 3H), 1.56-1.31 (m, 3H).

### Step-2: rac-(R)-2-(5-(2,6-dioxopiperidin-3-yl)pyridin-2-yl)-2-azaspiro[3.3]heptane-6-carbaldehyde

rac-(R)-3-{6-[6-(hydroxymethyl)-2-azaspiro[3.3]heptan-2-yl]pyridin-3-yl}piperidine-2,6-dione (45.00 mg, 0.1427 mmol) was dissolved in DMSO (0.5 mL) and then triethylamine (0.40 mL, 0.29 g, 2.8538 mmol) and sulfur trioxide pyridine complex (227.10 mg, 1.4269 mmol) were added. After stirring for 30 min, TLC showed no more starting material left. The solution was dissolved in DCM (30 mL). The solution was washed with water and brine, dried over sodium sulfate, filtered, and concentrated. The crude product was used in the next step without purification. LCMS: C₁₇H₁₉N₃O₃, requires: 313.36, found m/z=314.33 [M+H]⁺.

### Synthesis of 3-(4-(2,6-diazaspiro[3.3]heptan-2-yl)phenyl)piperidine-2,6-dione

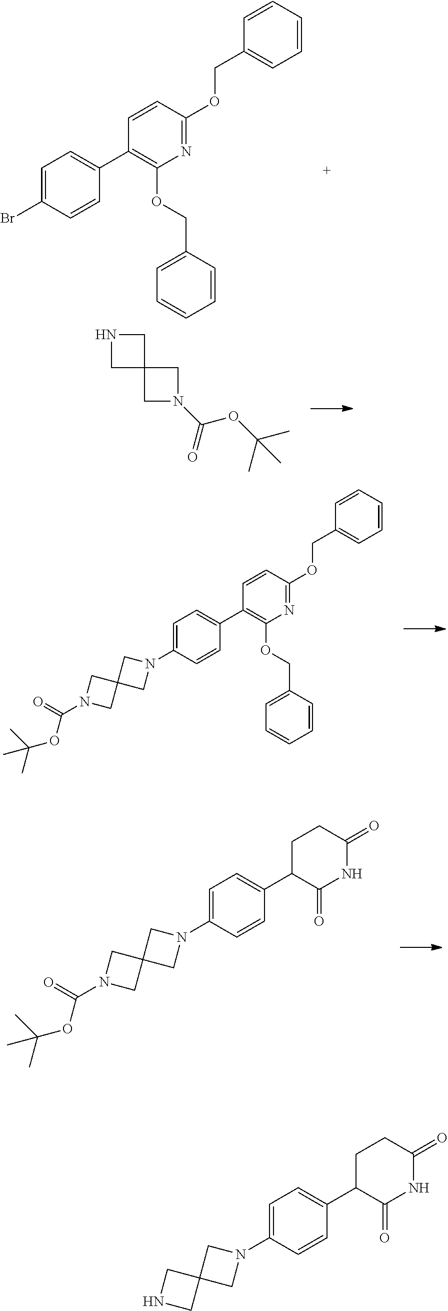

### Step-1: tert-butyl 6-(4-(2,6-bis(benzyloxy)pyridin-3-yl)phenyl)-2,6-diazaspiro[3.3]heptane-2-carboxylate

To a 20 mL vial was added 2,6-bis(benzyloxy)-3-(4-bromophenyl)pyridine (500.00 mg, 1.1202 mmol), tert-butyl 2,6-diazaspiro[3.3]heptane-2-carboxylate (244.31 mg, 1.2322 mmol), 1,3-bis[2,6-bis(pentan-3-yl)phenyl]-2H-imidazole, 3-chloropyridine, palladium chloride (44.46 mg, 0.0560 mmol), and cesium carbonate (1094.96 mg, 3.3606 mmol). Dioxane (4.00 mL) was then added and the mixture was purged with nitrogen gas for three minutes. Then the suspension was heated at 100° C. for sixteen hours. The crude reaction was diluted with EtOAc, washed with water twice, dried over Na₂SO₄, filtered, and concentrated. The crude product was loaded onto a Redi-Sep prepacked silica gel column (40 g column) eluting with EtOAc:hexane (0-70%) to provide tert-butyl 6-(4-(2,6-bis(benzyloxy)pyridin-3-yl)phenyl)-2,6-diazaspiro[3.3]heptane-2-carboxylate (0.443 g, 70.15%). LCMS: C₃₅H₃₇N₃O₄, requires: 563.70, found: m/z=564.52 [M+H]⁺.

### Step-2: tert-butyl 6-(4-(2,6-dioxopiperidin-3-yl)phenyl)-2,6-diazaspiro[3.3]heptane-2-carboxylate

To a mixture of tert-butyl 6-(4-(2,6-bis(benzyloxy)pyridin-3-yl)phenyl)-2,6-diazaspiro[3.3]heptane-2-carboxylate (443.00 mg, 0.7859 mmol) and palladium on carbon (150.00 mg) in THF (10.00 mL) was added isopropanol (3.00 mL) and the reaction mixture was stirred under a hydrogen balloon for 24 h. Pd/C solids were then filtered and the filtrate was washed with 10% MeOH in DCM and concentrated. The crude product was loaded onto a Redi-Sep prepacked silica gel column eluting with EtOAc in hexanes (70-100%) to afford tert-butyl 6-(4-(2,6-dioxopiperidin-3-yl)phenyl)-2,6-diazaspiro[3.3]heptane-2-carboxylate (0.22 g, 72.62%). LCMS: C₂₁H₂₇N₃O₄, requires: 385.2, found m/z=386.4 [M+H]⁺.

### Step-3: 3-(4-(2,6-diazaspiro[3.3]heptan-2-yl)phenyl)piperidine-2,6-dione

To a solution of tert-butyl 6-(4-(2,6-dioxopiperidin-3-yl)phenyl)-2,6-diazaspiro[3.3]heptane-2-carboxylate (150.00 mg, 0.3881 mmol) in DCM (2.00 mL) was added trifluoroacetic acid (2 mL). After stirring for one hour, TFA and DCM were evaporated. The product was lyophilized to dryness to provide 3-(4-(2,6-diazaspiro[3.3]heptan-2-yl)phenyl)piperidine-2,6-dione in quantitative yield. LCMS C₁₆H₁₉N₃O₂ requires: 285.1, found: m/z=286.3 [M+H]⁺.

### Synthesis of 3-(6-{2,6-diazaspiro[3.3]heptan-2-yl}pyridin-3-yl)piperidine-2,6-dione Synthesis

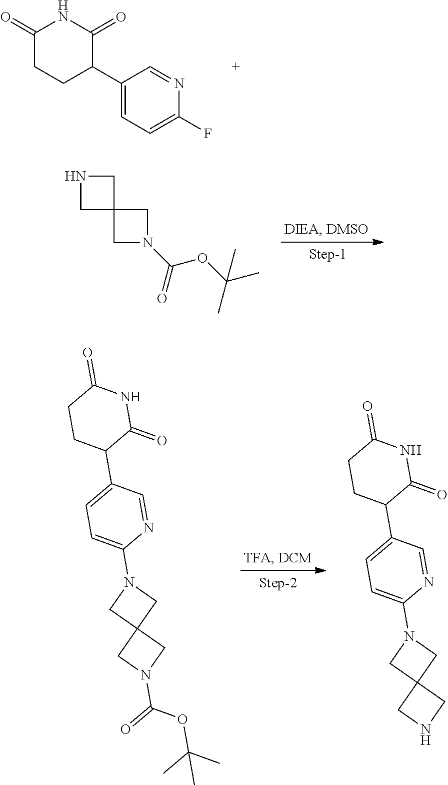 [SMILES: O=C1CCC(c2ccc(F)nc2)C(=O)N1 ; CC(C)(C)OC(=O)N1CC2(CNC2)C1 ; CC(C)(C)OC(=O)N1CC2(C1)CN(c1ccc(C3CCC(=O)NC3=O)cn1)C2 ; O=C1CCC(c2ccc(N3CC4(CNC4)C3)nc2)C(=O)N1]

### Step-1: Synthesis of tert-butyl 6-(5-(2,6-dioxopiperidin-3-yl)pyridin-2-yl)-2,6-diazaspiro[3.3]heptane-2-carboxylate

To a solution of 3-(6-fluoropyridin-3-yl)piperidine-2,6-dione (250.00 mg, 1.2008 mmol) and tert-butyl 2,6-diazaspiro[3.3]heptane-2-carboxylate (238.08 mg, 1.2008 mmol) in DMSO (2.00 mL), was added N,N-diisopropylethylamine (0.85 mL, 0.62 g, 4.8033 mmol) and the solution was heated at 120° C. for four hours. The crude product was lyophilized to remove DMSO. The resulting crude oil was loaded onto a Redi-Sep prepacked silica gel column eluting with 0-10% MeOH in EtOAc to afford tert-butyl 6-(5-(2,6-dioxopiperidin-3-yl)pyridin-2-yl)-2,6-diazaspiro[3.3]heptane-2-carboxylate (0.15 g, 32.32%). LCMS: C₂₁H₂₉N₃O₄ required: 386.2, found: m/z=386.5 [M+H]⁺.

### Step-2: Synthesis of 3-(6-{2,6-diazaspiro[3.3]heptan-2-yl}pyridin-3-yl)piperidine-2,6-dione

To a solution of tert-butyl 6-(5-(2,6-dioxopiperidin-3-yl)pyridin-2-yl)-2,6-diazaspiro[3.3]heptane-2-carboxylate (150.00 mg, 0.3881 mmol) in DCM (2.00 mL) was added trifluoroacetic acid (2 mL) and the resulting mixture was stirred for one hour. Solvent was removed under reduced pressure, and the product was lyophilized to afford 3-(6-{2,6-diazaspiro[3.3]heptan-2-yl}pyridin-3-yl)piperidine-2,6-dione as a TFA salt in quantitative yield. LCMS: C₁₅H₁₈N₄O₂ required: 286.1, found: m/z=286.3 [M+H]⁺.

### Amide Bond Formation Between the Hook Amines and the Harness Acids

FluidX barcoded tubes containing harness acid stocks in NMP (0.1 M, eq=10 μmol) were retrieved from the Hamilton Storage Q20 unit and placed onto the deck of a 1.3 m Hamilton Robotics Vantage. The hook amines were dissolved in NMP (0.1 M) containing DIEA (0.15 M, 1.5 equiv) in V-bottom 20 mL vials and placed into vial racks onto the deck. Transfers of 100 μL from the harness acid stocks into corresponding wells of 96 deep well plates were performed, followed by addition of each of DIEA (0.15 M, 1.5 equiv) and HATU (0.1 M, 1 equiv) cocktails from 60 mL troughs. The plates were shaken ten minutes at 600 rpm for activation. Transfers of 100 μL from the hook stocks into corresponding wells of the same plates were performed and then the plates were sealed and shaken overnight at 600 rpm for amide bond formation. Finally, these plates were submitted for RP-HPLC purification.

### Reductive Amination Between the Hook Amines and the Harness Aldehydes

FluidX barcoded tubes containing harness aldehyde stocks in NMP (0.1 M, eq=10 μmol) were retrieved from the Hamilton Storage Q20 unit and placed onto the deck of a 1.3 m Hamilton Robotics Vantage. The hook amines were dissolved in dry NMP (0.1 M) containing DIEA (0.2 M, 2 equiv) and AcOH (0.2 equiv, 0.02 M) in V-bottom 20 mL vials and placed into vial racks onto the deck. Transfers of 100 μL from the harness stocks into corresponding wells of 96 deep well plates were performed, followed by transfers of 100 μL from the hook stocks into corresponding wells of the same 96 deep well plates. The plates were then shaken ninety minutes at 600 rpm for Schiff base formation. Additional plates dry-loaded with polymer-supported BH₃CN (20 mg, 4 equiv) in each well were placed on the deck and the reactions were transferred into these corresponding plates using the 96 pipettor head. The plates were then sealed and shaken overnight at 600 rpm for the reductive amination to occur. The plates were then unsealed, and the reaction mixture was separated from the polymer-supported BH₃CN by decantation followed by washing cycles with fresh solvent from 60 mL troughs to recover only the liquid phase into new plates. Finally, these plates were submitted for RP-HPLC purification.

### Synthesis of 1-(4-(2,6-dioxopiperidin-3-yl)-2-fluorophenyl)piperidine-4-carbaldehyde (HA-3)

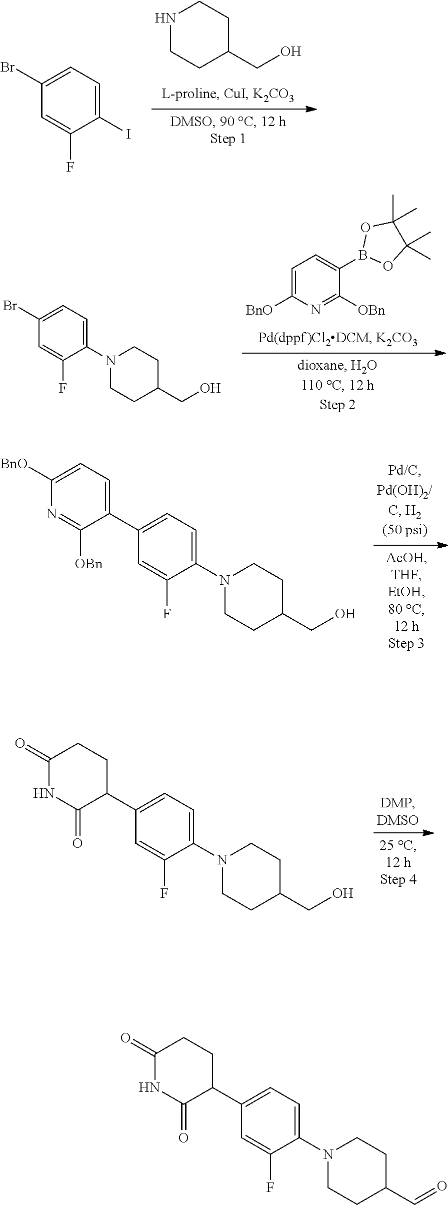

### Step-1: Synthesis of (1-(4-bromo-2-fluorophenyl)piperidin-4-yl)methanol

To a solution of 4-bromo-2-fluoro-iodobenzene (42.1 g, 365 mmol, 1.10 equiv) and piperidin-4-ylmethanol (100 g, 332 mmol, 1.00 equiv) in DMSO (1000 mL) was added L-proline (17.4 g, 132 mmol, 0.400 equiv), K₂CO₃ (91.8 g, 664 mmol, 2.00 equiv) and CuI (12.6 g, 66.4 mmol, 0.200 equiv) under a nitrogen atmosphere. The reaction was stirred at 90° C. for 12 h. The reaction mixture was then cooled to 20° C. and poured into saturated NH₄Cl solution (1000 mL). The product was extracted with ethyl acetate (1000 mL×3) and the combined organic layers were washed with brine (1000 mL), dried over Na₂SO₄, filtered, and concentrated. The residue was purified by column chromatography (SiO₂, petroleum ether:ethyl acetate=10:1 to 0:1, R_{f}=0.55). The desired product (10.7 g, 35.6 mmol, 10.7% yield) was obtained as a yellow solid. LCMS: m/z=290.0 (M+H)⁺.

### Step-2: Synthesis of (1-(4-(2,6-bis(benzyloxy)pyridin-3-yl)-2-fluorophenyl)piperidin-4-yl)methanol

To a solution of (1-(4-bromo-2-fluorophenyl)piperidin-4-yl)methanol (11.6 g, 38.6 mmol, 1.00 equiv), 2,6-bis(benzyloxy)-3-(4,4,5,5-tetramethyl-1,3,2-dioxaborolan-2-yl)pyridine (17.7 g, 42.4 mmol, 1.10 equiv), K₂CO₃ (16.0 g, 115 mmol, 3.00 equiv) in dioxane (116 mL) and H₂O (23 mL) was added Pd(dppf)Cl₂·CH₂Cl₂ (3.15 g, 3.86 mmol, 0.100 equiv) under a nitrogen atmosphere. Then the reaction mixture was stirred at 110° C. for 12 h. The mixture was then cooled to 25° C. Then the reaction mixture was poured into H₂O (300 mL) and was extracted with EtOAc (200 mL×3). The combined organic layers were washed with brine (200 mL), dried over Na₂SO₄, and concentrated. The crude product was purified by MPLC (SiO2, petroleum ether:ethyl acetate=100:1 to 5:1), R_{f}=0.5 (petroleum ether:ethyl acetate=1:1). (1-(4-(2,6-bis(benzyloxy)pyridin-3-yl)-2-fluorophenyl)piperidin-4-yl)methanol (10.7 g, 20.5 mmol, 53.2% yield) was obtained as a light yellow solid. LCMS: m/z=499.4 (M+H)⁺.

### Step-3: Synthesis of 3-(3-fluoro-4-(4-(hydroxymethyl)piperidin-1-yl)phenyl)piperidine-2,6-dione

To a solution of (1-(4-(2,6-bis(benzyloxy)pyridin-3-yl)-2-fluorophenyl)piperidin-4-yl)methanol (11.6 g, 23.2 mmol, 1.00 equiv) in THF (120 mL) and EtOH (120 mL) was added AcOH (4.19 g, 69.8 mmol, 3.99 mL, 3.00 equiv), Pd/C (2.90 g, 10% purity) and Pd(OH)₂/C (3.01 g, 4.29 mmol, 20% purity) under a nitrogen atmosphere. The suspension was degassed under vacuum and purged with H₂ several times. The mixture was stirred under H₂ (50 psi) at 80° C. for 12 h. The reaction was then cooled to 20° C. and filtered. The filtrate was concentrated. The desired product (8.40 g, crude) was obtained as a yellow solid and used in the next step without further purification. LCMS: m/z=321.2 (M+H)⁺.

### Step-4: Synthesis of 1-(4-(2,6-dioxopiperidin-3-yl)-2-fluorophenyl)piperidine-4-carbaldehyde

To a solution of 3-(3-fluoro-4-(4-(hydroxymethyl)piperidin-1-yl)phenyl)piperidine-2,6-dione (8.40 g, 26.2 mmol, 1.00 equiv) in DMSO (90.0 mL) was added DMP (22.2 g, 52.4 mmol, 16.2 mL, 2.00 equiv) in portions. The reaction was stirred at 25° C. for 12 h. The pH of the reaction mixture then was adjusted to pH=10 with saturated aqueous Na₂CO₃ and the aqueous layer was extracted with ethyl acetate (400 mL×4). The combined organic layers were washed with Na₂S₂O₃ solution (500 mL×2) and brine (1000 mL), dried over Na₂SO₄, filtered, and concentrated under vacuum. The crude product was triturated with ethyl acetate (100 mL) at 25° C. for 12 h. 1-(4-(2,6-dioxopiperidin-3-yl)-2-fluorophenyl)piperidine-4-carbaldehyde (2.50 g, 7.60 mmol, 28.9% yield) was obtained as a yellow solid. LCMS: m/z=317.1 (M−H)⁺. ¹H NMR (400 MHz, DMSO-d₆) δ 10.8 (s, 1H), 9.64 (s, 1H), 7.06-6.90 (s, 3H), 3.83-3.76 (m, 1H), 3.29-3.24 (m, 2H), 2.76 (t, J=10.0 Hz, 2H), 2.69-2.60 (m, 1H), 2.48-2.41 (m, 2H), 2.24-2.13 (m, 1H), 2.04-1.91 (m, 3H), 1.71-1.58 (m, 2H).

### Synthesis of 1-(4-(2,6-dioxopiperidin-3-yl)-3-fluorophenyl)piperidine-4-carbaldehyde (HA-4)

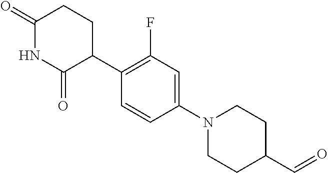 [SMILES: O=CC1CCN(c2ccc(C3CCC(=O)NC3=O)c(F)c2)CC1]

HA-4 was synthesized following the same procedure as HA-3 except substituting 4-bromo-2-fluoro-1-iodobenzene with 1-bromo-2-fluoro-4-iodobenzene in Step 1 to give 1-(4-(2,6-dioxopiperidin-3-yl)-3-fluorophenyl)piperidine-4-carbaldehyde. LCMS: m/z=317.1 (M−H)⁺. ¹H NMR (400 MHz, DMSO-d₆) δ 10.79 (s, 1H), 9.62 (s, 1H), 7.07 (t, J=8.8 Hz, 1H), 6.77-6.69 (m, 2H), 3.91-3.84 (m, 1H), 3.67-3.59 (m, 2H), 2.91-2.81 (m, 2H), 2.76-2.65 (m, 1H), 2.56-2.52 (m, 1H), 2.49-2.45 (m, 1H), 2.20-2.07 (m, 1H), 1.97-1.86 (m, 3H), 1.61-1.49 (m, 2H).

### Synthesis of 1-(4-(2,6-dioxopiperidin-3-yl)-3,5-difluorophenyl)piperidine-4-carbaldehyde (HA-5)

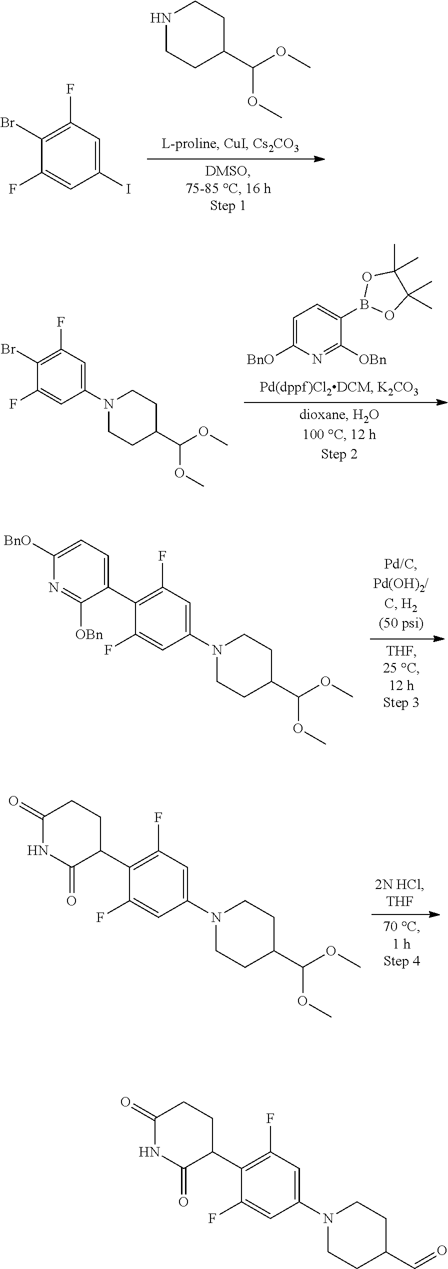

### Step 1: Synthesis of 1-(4-bromo-3,5-difluorophenyl)-4-(dimethoxymethyl)piperidine

2-bromo-1,3-difluoro-5-iodobenzene (56.3 g, 176 mmol, 1.00 equiv), 4-(dimethoxymethyl)piperidine (30.9 g, 194 mmol, 1.10 equiv), L-proline (8.13 g, 70.6 mmol, 0.40 equiv), Cs₂CO₃ (115 g, 353 mmol, 2.00 equiv), and CuI (6.72 g, 35.3 mmol, 0.20 equiv) was stirred in DMSO (350 mL) at 20° C. and purged with N₂ three times. The mixture was then stirred at 85° C. for 16 h under a nitrogen atmosphere. LCMS showed 60.5% of the desired mass (Rₜ=0.715 min) was detected. TLC (Petroleum ether:Ethyl acetate=10:1) showed that 2-bromo-1,3-difluoro-5-iodobenzene (R_{f}=0.8) was consumed and a major spot (R_{f}=0.3) was detected. The reaction was then added to water (800 mL) and extracted with ethyl acetate (300 mL×3). The combined organic layers were washed with brine (1.00 L×1), dried over Na₂SO₄, filtered, and concentrated. The residue was purified by column chromatography (SiO₂, Ethyl acetate:Petroleum ether=0:1 to 1:20; Ethyl acetate:Petroleum ether=1:10, R_{f}=0.3). Then the combined fractions were concentrated under vacuum. 1-(4-bromo-3,5-difluorophenyl)-4-(dimethoxymethyl)piperidine (30.0 g, 84.1 mmol, 48.5% yield, 98.2% purity) was obtained as a light-yellow solid. LCMS: m/z=350.2 (M+H)⁺. ¹H NMR (400 MHz, CDCl₃) δ 6.47 (d, J=10.0 Hz, 2H), 4.07 (d, J=6.8 Hz, 1H), 3.74-3.62 (m, 2H), 3.38 (s, 6H), 2.84-2.68 (m, 2H), 1.89-1.77 (m, 3H), 1.52-1.30 (m, 2H).

### Step 2: Synthesis of 2,6-bis(benzyloxy)-3-(4-(4-(dimethoxymethyl)piperidin-1-yl)-2,6-difluorophenyl)pyridine

1-(4-bromo-3,5-difluorophenyl)-4-(dimethoxymethyl)piperidine (20.0 g, 57.1 mmol, 1.00 equiv), 2,6-bis(benzyloxy)-3-(4,4,5,5-tetramethyl-1,3,2-dioxaborolan-2-yl)pyridine (30.9 g, 74.2 mmol, 1.30 equiv), K₂CO₃ (23.6 g, 171 mmol, 3.00 equiv), and Pd(dppf)Cl₂·DCM(2.51 g, 3.43 mmol, 0.06 equiv) was added to dioxane (200 mL) and H₂O (40 mL) at 20° C. and purged with nitrogen three times. The mixture was then stirred at 100° C. for 16 h under a nitrogen atmosphere. LCMS showed 67.7% of the desired mass (Rₜ=0.796 min) was detected. TLC (Petroleum ether:Ethyl acetate=10:1) showed that 1-(4-bromo-3,5-difluorophenyl)-4-(dimethoxymethyl)piperidine (R_{f}=0.3) was consumed and a major spot (R_{f}=0.25) was detected. The reaction was then added to water (1.00 L) and extracted with ethyl acetate (300 mL×3). The combined organic layers were washed with brine (1.00 L×1), dried over Na₂SO₄, filtered, and concentrated. The residue was purified by column chromatography (SiO₂, Ethyl acetate:Petroleum ether=0:1 to 1:20; Ethyl acetate:Petroleum ether=1:10, R_{f}=0.3). Then the fractions were concentrated under vacuum. 2,6-bis(benzyloxy)-3-(4-(4-(dimethoxymethyl)piperidin-1-yl)-2,6-difluorophenyl)pyridine (20.0 g, 34.0 mmol, 59.6% yield, 95.5% purity) was obtained as a yellow solid. LCMS: m/z=561.3 (M+H)⁺. ¹H NMR (400 MHz, CDCl₃) δ 7.56-7.29 (m, 11H), 6.50-6.44 (m, 3H), 5.39-5.32 (m, 4H), 4.09 (d, J=7.0 Hz, 1H), 3.75-3.71 (m, 2H), 3.39 (s, 6H), 2.79-2.73 (m, 2H), 1.93-1.72 (m, 3H), 1.56 (s, 2H).

### Step 3: Synthesis of 3-(4-(4-(dimethoxymethyl)piperidin-1-yl)-2,6-difluorophenyl)piperidine-2,6-dione

To a solution of 2,6-bis(benzyloxy)-3-(4-(4-(dimethoxymethyl)piperidin-1-yl)-2,6-difluorophenyl)pyridine (5.00 g, 8.92 mmol, 1.00 equiv) in THF (50.0 mL) was added Pd/C (1.50 g, 1.41 mmol, 10% purity) and Pd(OH)₂/C (1.50 g, 3.20 mmol, 30% purity) under a nitrogen atmosphere. The suspension was degassed and purged with H₂ three times. The mixture was then stirred under H₂ (50 psi) at 25° C. for 12 h. LCMS showed that the desired peak (Rₜ=0.568 min) was detected and the starting material was consumed completely. The mixture was then filtered and concentrated. 3-(4-(4-(dimethoxymethyl)piperidin-1-yl)-2,6-difluorophenyl)piperidine-2,6-dione (2.40 g, 6.28 mmol, 70.3% yield) was obtained as an off-white solid. LCMS: m/z=383.2 (M+H)⁺. ¹H NMR (400 MHz, CDCl₃) δ 8.04 (s, 1H), 6.42 (d, J=12.1 Hz, 2H), 4.07 (d, J=6.6 Hz, 1H), 4.01-3.88 (m, 1H), 3.69 (br d, J=12.6 Hz, 2H), 3.38 (s, 6H), 2.88-2.61 (m, 4H), 2.41-2.24 (m, 1H), 2.21-2.06 (m, 1H), 1.92-1.77 (m, 3H), 1.52-1.31 (m, 2H).

### Step 4: Synthesis of 1-(4-(2,6-dioxopiperidin-3-yl)-3,5-difluorophenyl)piperidine-4-carbaldehyde

To a solution of 3-(4-(4-(dimethoxymethyl)piperidin-1-yl)-2,6-difluorophenyl)piperidine-2,6-dione (20.0 g, 52.3 mmol, 1.00 equiv) in THF (460 mL) was added HCl (2N, 465 mL, 17.8 equiv) dropwise slowly. The reaction solution was then stirred at 70° C. for one hour. LCMS showed 100% of the desired mass (Rₜ=0.535 min) was detected. The reaction mixture was then treated with saturated NaHCO₃ solution until pH=7, extracted with methyl tetrahydrofuran (500 mL×3), dried over Na₂SO₄, filtered, and concentrated to provide a crude product. The crude was triturated with MTBE (30.0 mL) at 25° C. for 30 min. 1-(4-(2,6-dioxopiperidin-3-yl)-3,5-difluorophenyl)piperidine-4-carbaldehyde (15.4 g, 42.9 mmol, 84.2% yield, 96.2% purity) was obtained as a white solid. LCMS: m/z=337.2 (M+H)⁺. ¹H NMR (400 MHz, DMSO-d₆) δ 10.88 (s, 1H), 9.62 (s, 1H), 6.64 (d, J=12.6 Hz, 2H), 4.10-4.00 (m, 1H), 3.73-3.62 (m, 2H), 2.99-2.87 (m, 2H), 2.85-2.72 (m, 1H), 2.61-2.50 (m, 2H), 2.19-2.05 (m, 1H), 2.01-1.83 (m, 3H), 1.62-1.46 (m, 2H).

### Synthesis of 1-(4-(2,6-dioxopiperidin-3-yl)-2,5-difluorophenyl)piperidine-4-carbaldehyde (HA-6)

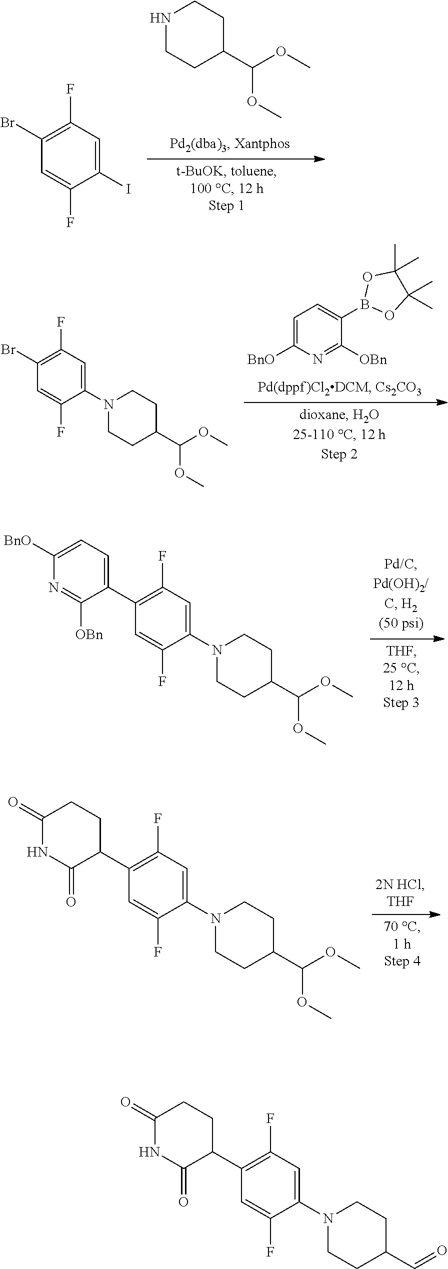

### Step 1: Synthesis of 1-(4-bromo-2,5-difluorophenyl)-4-(dimethoxymethyl)piperidine

To a solution of 1-bromo-2,5-difluoro-4-iodobenzene (30.0 g, 94.1 mmol, 1.00 equiv), 4-(dimethoxymethyl)piperidine (18.0 g, 113 mmol, 1.20 equiv), Xantphos (10.9 g, 18.8 mmol, 0.200 equiv), and t-BuOK (13.7 g, 122 mmol, 1.30 equiv) in toluene (210 mL) was added Pd₂(dba)₃ (8.61 g, 9.41 mmol, 0.100 equiv) under N₂. The reaction was then stirred at 100° C. for 12 h. The mixture was then diluted with H₂O (500 mL) and extracted with EtOAc (500 mL×2). The combined organic layer was washed with brine (500 mL), dried over Na₂SO₄, filtered, and concentrated under reduced pressure. The crude was purified by column chromatography (SiO₂, Petroleum ether:Ethyl acetate=0:1 to 10:1; Petroleum ether:Ethyl acetate=81, R_{f}=0.4) and concentrated to provide 1-(4-bromo-2,5-difluorophenyl)-4-(dimethoxymethyl)piperidine (15.3 g, 40.3 mmol, 42.8% yield, 92.2% purity) as a yellow oil. LCMS: m/z=352.0 (M+H)⁺. ¹H NMR (400 MHz, CDCl₃) δ 7.18 (dd, J=6.4, 11.6 Hz, 1H), 6.71 (dd, J=7.6, 10.4 Hz, 1H), 4.10 (d, J=7.2 Hz, 1H), 3.46 (d, J=11.6 Hz, 2H), 3.38 (s, 6H), 2.62 (dt, J=2.0, 12.0 Hz, 2H), 1.85 (br d, J=13.2 Hz, 2H), 1.81-1.69 (m, 1H), 1.57-1.43 (m, 2H).

### Step 2: Synthesis of 2,6-bis(benzyloxy)-3-(4-(4-(dimethoxymethyl)piperidin-1-yl)-2,5-difluorophenyl)pyridine

To a solution of 1-(4-bromo-2,5-difluorophenyl)-4-(dimethoxymethyl)piperidine (15.3 g, 40.3 mmol, 1.00 equiv) in dioxane (300 mL) and H₂O (60.0 mL) was added 2,6-bis(benzyloxy)-3-(4,4,5,5-tetramethyl-1,3,2-dioxaborolan-2-yl)pyridine (23.8 g, 52.4 mmol, 1.30 equiv), Cs₂CO₃ (32.8 g, 101 mmol, 2.50 equiv), and Pd(dppf)Cl₂·CH₂Cl₂ (1.64 g, 2.01 mmol, 0.05 equiv) at 25° C. under N₂. The reaction mixture was then stirred at 100° C. for 12 h. The mixture was then poured into H₂O (100 mL) and extracted with EtOAc (50.0 mL×3). The combined organic layer was washed with brine (100 mL), dried over Na₂SO₄, filtered, and concentrated under reduced pressure. The crude was purified by MPLC (SiO₂, Petroleum ether:Ethyl acetate=1:0 to 10:1; Petroleum ether:Ethyl acetate=5:1, R_{f}=0.4) and concentrated to provide 2,6-bis(benzyloxy)-3-(4-(4-(dimethoxymethyl)piperidin-1-yl)-2,5-difluorophenyl)pyridine (21.5 g, 37.0 mmol, 91.9% yield, 96.5% purity) as a yellow oil. LCMS: m/z=561.3 (M+H)⁺. ¹H NMR (400 MHz, CDCl₃) δ 7.54 (dd, J=1.2, 8.0 Hz, 1H), 7.45-7.41 (m, 2H), 7.40-7.28 (m, 8H), 7.09 (dd, J=7.2, 13.2 Hz, 1H), 6.78-6.65 (m, 1H), 6.46 (d, J=8.0 Hz, 1H), 5.41 (s, 2H), 5.36 (s, 2H), 4.12 (d, J=7.2 Hz, 1H), 3.54 (d, J=12.0 Hz, 2H), 3.40 (s, 6H), 2.66 (br t, J=11.6 Hz, 2H), 2.06 (s, 1H), 1.87 (br d, J=12.8 Hz, 2H), 1.81-1.73 (m, 1H), 1.58-1.47 (m, 2H).

### Step 3: Synthesis of 3-(4-(4-(dimethoxymethyl)piperidin-1-yl)-2,5-difluorophenyl)piperidine-2,6-dione

To a solution of 2,6-bis(benzyloxy)-3-(4-(4-(dimethoxymethyl)piperidin-1-yl)-2,5-difluorophenyl)pyridine (21.5 g, 37.0 mmol, 1.00 equiv) in THF (360 mL) was added Pd/C (6.45 g, 10% purity) and Pd(OH)₂/C (6.45 g, 20% purity) under N₂. The suspension was then degassed and purged with H₂ three times. The mixture was then stirred under H₂ (50 psi) at 25° C. for 12 h. LCMS showed the starting material was consumed and desired mass (Rₜ=0.745 min) was formed. The mixture was then filtered through celite and the filtrate was concentrated under reduced pressure. The crude was triturated with MTBE (100 mL) at 25° C. for 0.5 h, filtered, and the cake was dried under reduced pressure to provide 3-(4-(4-(dimethoxymethyl)piperidin-1-yl)-2,5-difluorophenyl)piperidine-2,6-dione (14.0 g, 36.6 mmol, 98.9% yield, 100% purity) as a white solid. LCMS: m/z=383.1 (M+H)⁺. ¹H NMR (400 MHz, CDCl₃) δ 8.20-8.01 (m, 1H), 6.82 (dd, J=6.8, 12.4 Hz, 1H), 6.69 (dd, J=7.2, 11.6 Hz, 1H), 4.10 (d, J=7.2 Hz, 1H), 3.81 (dd, J=5.6, 11.2 Hz, 1H), 3.49 (br d, J=11.2 Hz, 2H), 3.38 (s, 6H), 2.86-2.75 (m, 1H), 2.74-2.56 (m, 3H), 2.33-2.13 (m, 2H), 1.84 (br d, J=13.2 Hz, 2H), 1.77-1.71 (m, 1H), 1.58-1.43 (m, 2H).

### Step 4: Synthesis of 1-(4-(2,6-dioxopiperidin-3-yl)-2,5-difluorophenyl)piperidine-4-carbaldehyde

To a solution of 3-(4-(4-(dimethoxymethyl)piperidin-1-yl)-2,5-difluorophenyl)piperidine-2,6-dione (10.0 g, 26.2 mmol, 1.00 equiv) in THF (250 mL) was added HCl (2N, 233 mL, 17.8 equiv). The reaction solution was then stirred at 70° C. for one hour. The reaction mixture was then cooled to 25° C. and saturated NaHCO₃ solution was added until pH=7. The mixture was then extracted with EtOAc (300 mL×3) and the combined organic layer was washed with brine (500 mL), dried over Na₂SO₄, filtered, and concentrated to provide 1-(4-(2,6-dioxopiperidin-3-yl)-2,5-difluorophenyl)piperidine-4-carbaldehyde (7.68 g, 22.8 mmol, 87.1% yield, 99.7% purity) as an off-white solid. LCMS: m/z=337.1 (M+H)⁺.

¹H NMR (400 MHz, CDCl₃) δ 9.72 (s, 1H), 8.12 (br s, 1H), 6.84 (dd, J=6.8, 12.4 Hz, 1H), 6.68 (dd, J=7.2, 11.6 Hz, 1H), 3.82 (dd, J=5.6, 11.2 Hz, 1H), 3.48-3.34 (m, 2H), 2.92-2.75 (m, 3H), 2.74-2.61 (m, 1H), 2.49-2.36 (m, 1H), 2.33-2.14 (m, 2H), 2.10-2.00 (m, 2H), 1.94-1.78 (m, 2H).

### Synthesis of 1-(4-(2,6-dioxopiperidin-3-yl)-2,3-difluorophenyl)piperidine-4-carbaldehyde (HA-7)

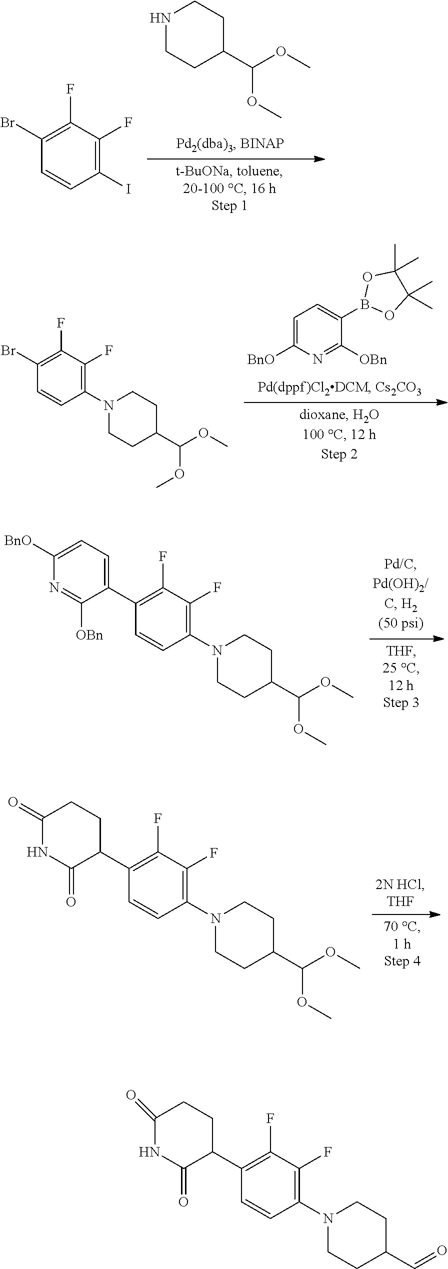

### Step 1: Synthesis of 1-(4-bromo-2,3-difluorophenyl)-4-(dimethoxymethyl)piperidine

1-Bromo-2,3-difluoro-4-iodobenzene (20.0 g, 62.7 mmol, 1.00 equiv), 4-(dimethoxymethyl)piperidine (10.9 g, 68.9 mmol, 1.10 equiv), BINAP (1.56 g, 2.51 mmol, 0.04 equiv), t-BuONa (12.0 g, 125 mmol, 2.00 equiv), and Pd₂(dba)₃ (1.15 g, 1.25 mmol, 0.02 equiv) was added to toluene (200 mL) at 20° C. and the mixture was purged with N₂ three times. The mixture was then stirred at 100° C. for 16 h. The mixture was then concentrated directly via vacuum. The residue was purified by column chromatography (SiO₂, Ethyl acetate:Petroleum ether=0:1 to 1:20; Ethyl acetate:Petroleum ether=1:10, R_{f}=0.30). Then the fractions were concentrated under vacuum. 1-(4-bromo-2,3-difluorophenyl)-4-(dimethoxymethyl)piperidine (14.0 g, 31.5 mmol, 50.3% yield, 79.0% purity) was obtained as a yellow solid. LCMS: m/z=350.1 (M+H)⁺. ¹H NMR (400 MHz, CDCl₃) δ 7.21-7.12 (m, 1H), 6.67-6.55 (m, 1H), 4.10 (d, J=7.1 Hz, 1H), 3.46 (br d, J=12.0 Hz, 2H), 3.38 (s, 6H), 2.73-2.60 (td, 2H), 1.85 (br d, J=13.9 Hz, 2H), 1.56-1.43 (m, 2H), 1.26 (t, 1H).

### Step 2: Synthesis of 2,6-bis(benzyloxy)-3-(4-(4-(dimethoxymethyl)piperidin-1-yl)-2,3-difluorophenyl)pyridine

1-(4-bromo-2,3-difluorophenyl)-4-(dimethoxymethyl)piperidine (13.0 g, 29.7 mmol, 1.00 equiv), 2,6-bis(benzyloxy)-3-(4,4,5,5-tetramethyl-1,3,2-dioxaborolan-2-yl)pyridine (16.1 g, 38.6 mmol, 1.30 equiv), Cs₂CO₃ (24.1 g, 74.2 mmol, 2.50 equiv), and Pd(dppf)Cl₂·CH₂Cl₂ (1.21 g, 1.48 mmol, 0.05 equiv) was added into dioxane (260 mL) and H₂O (52.0 mL) at 20° C. and the mixture was purged with N₂ three times. The mixture was then stirred at 100° C. for 16 h under a nitrogen atmosphere. The residue was then added to water (200 mL) and extracted with ethyl acetate (60.0 mL×3). The combined organic layers were washed with brine (100 mL×1), dried over Na₂SO₄, filtered, and concentrated. The residue was purified by column chromatography (SiO₂, Ethyl acetate:Petroleum ether=0:1 to 1:20; Ethyl acetate:Petroleum ether=1:10, R_{f}=0.25). Then the fractions were concentrated under vacuum. 2,6-bis(benzyloxy)-3-(4-(4-(dimethoxymethyl)piperidin-1-yl)-2,3-difluorophenyl)pyridine (8.80 g, 13.8 mmol, 46.5% yield, 88.0% purity) was obtained as a yellow solid. LCMS: m/z=561.3 (M+H)⁺. ¹H NMR (400 MHz, CDCl₃) δ 7.56-7.29 (m, 11H), 7.10-6.95 (m, 1H), 6.46 (d, J=8.1 Hz, 1H), 5.46-5.23 (m, 4H), 4.12 (d, J=7.2 Hz, 1H), 3.54 (br d, J=11.9 Hz, 2H), 3.40 (s, 6H), 2.71 (t, J=11.4 Hz, 2H), 1.87 (br d, J=12.8 Hz, 2H), 1.81-1.71 (m, 1H), 1.64-1.48 (m, 3H).

### Step 3: Synthesis of 3-(4-(4-(dimethoxymethyl)piperidin-1-yl)-2,3-difluorophenyl)piperidine-2,6-dione

To a solution of 2,6-bis(benzyloxy)-3-(4-(4-(dimethoxymethyl)piperidin-1-yl)-2,3-difluorophenyl)pyridine (43.0 g, 76.7 mmol, 1.00 equiv) in THF (430 mL) was added Pd/C (12.9 g, 12.1 mmol, 10% purity) and Pd(OH)₂/C (12.9 g, 27.5 mmol, 30% purity) under a nitrogen atmosphere. The suspension was then degassed and purged with H₂ three times. The mixture was then stirred under H₂ (50 psi) at 25° C. for 6 h. The mixture was then filtered and concentrated. The crude was triturated with MTBE (20.0 mL) at 25° C. for 30 min, filtered, and the cake was dried under reduced pressure to provide 3-(4-(4-(dimethoxymethyl)piperidin-1-yl)-2,3-difluorophenyl)piperidine-2,6-dione (8.00 g, 20.9 mmol, 88.8% yield) as a white solid. LCMS: m/z=383.1 (M+H)⁺. ¹H NMR (400 MHz, DMSO-d₆) δ 10.92 (s, 1H), 7.18-6.69 (m, 2H), 4.16 (d, J=6.5 Hz, 1H), 4.11-4.00 (m, 1H), 3.47-3.38 (m, 3H), 3.32 (s, 6H), 2.85-2.64 (m, 3H), 2.30-2.13 (m, 1H), 2.10-1.95 (m, 1H), 1.84-1.68 (m, 3H), 1.52-1.35 (m, 2H).

### Step 4: Synthesis of 1-(4-(2,6-dioxopiperidin-3-yl)-2,3-difluorophenyl)piperidine-4-carbaldehyde

To a solution of 3-(4-(4-(dimethoxymethyl)piperidin-1-yl)-2,3-difluorophenyl)piperidine-2,6-dione (4.00 g, 10.4 mmol, 1.00 equiv) in THF (93.0 mL) was added HCl (2.00N, 93.1 mL, 17.8 equiv) dropwise slowly. After addition, the reaction solution was then stirred at 70° C. for one hour. The reaction mixture was then treated with saturated NaHCO₃ solution until pH=7, extracted with EtOAc (30.0 mL×3), dried over Na₂SO₄, filtered, and concentrated to provide crude material. The crude was triturated with EtOAc (10.0 mL) at 25° C. for 30 min. 1-(4-(2,6-dioxopiperidin-3-yl)-2,3-difluorophenyl)piperidine-4-carbaldehyde (2.00 g, 5.86 mmol, 56.0% yield, 98.4% purity) was obtained as a white solid. LCMS: m/z=337.2 (M+H)⁺. ¹H NMR (400 MHz, DMSO-d₆) δ 10.88 (s, 1H), 9.64 (s, 1H), 6.99 (br t, J=7.5 Hz, 1H), 6.82 (br t, J=7.9 Hz, 1H), 4.12-3.94 (m, 1H), 3.35-3.26 (m, 2H), 2.88-2.67 (m, 3H), 2.55 (br d, J=3.1 Hz, 1H), 2.49-2.43 (m, 1H), 2.26-2.09 (m, 1H), 2.05-1.88 (m, 3H), 1.73-1.56 (m, 2H).

### Synthesis of 1-(5-(2,4-dioxotetrahydropyrimidin-1(2H)-yl) 679 pyridine-2-yl)piperidine-4-carbaldehyde (HA-8)

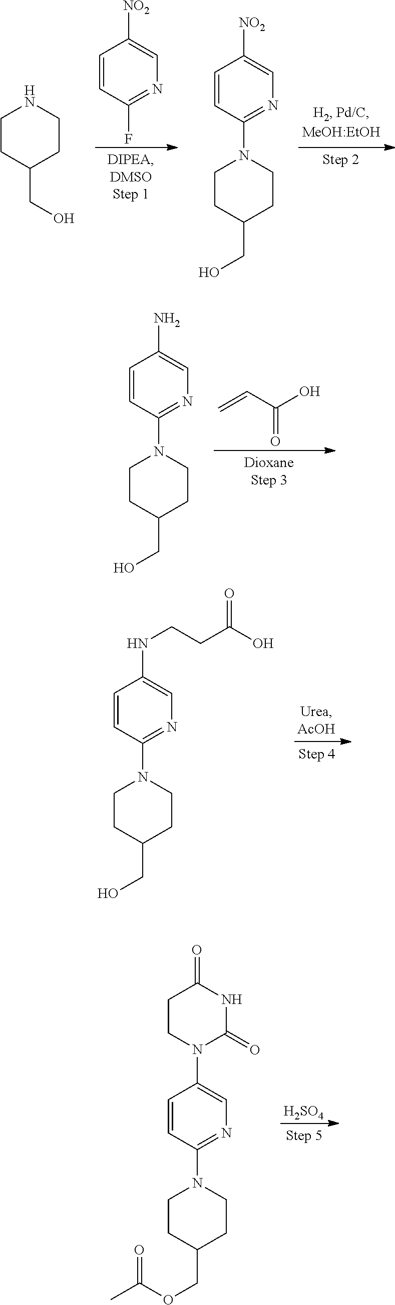

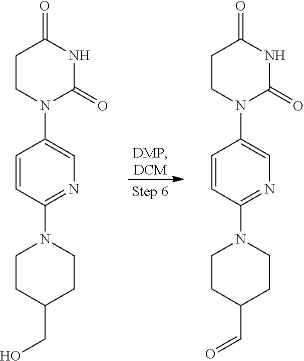

### Step-1: Synthesis of (1-(5-nitropyridin-2-yl)piperidin-4-yl)methanol

A mixture of 2-fluoro-4-nitropyridine (20 g, 0.140 mol, 1 equiv), piperidin-4-ylmethanol (24.318 g, 0.211 mol, 1.5 equiv), and DIPEA (27.29 mL, 0.211 mol, 1.5 equiv) in anhydrous DMSO (50 mL, 2.82 M) was stirred at 90° C. overnight. Then the reaction mixture was poured into ice water. The resulting precipitate was filtered and purified by flash chromatography (DCM:MeOH=9:1) to provide [1-(5-nitropyridin-2-yl)piperidin-4-yl]methanol (33.37 g, 47% yield) as a yellow solid. LCMS: m/z=237.7 (M+H)⁺. ¹H NMR (300 MHz, CDCl3) δ 9.05 (d, J=2.8 Hz, 1H), 8.20 (dd, J=9.6, 2.8 Hz, 1H), 6.60 (d, J=9.6 Hz, 1H), 4.59 (d, J=13.4 Hz, 2H), 3.57 (d, J=5.9 Hz, 2H), 3.11-2.95 (m, 2H), 1.98-1.84 (m, 3H), 1.31 (tdd, J=14.3, 11.8, 4.2 Hz, 2H).

### Step-2: Synthesis of (1-(5-aminopyridin-2-yl)piperidin-4-yl)methanol

[1-(5-nitropyridin-2-yl)piperidin-4-yl]methanol (15.5 g, 0.065 mol, 1 equiv) was dissolved in a mixture of EtOH:MeOH (1:1) (250 mL, 0.26 M), degassed, and charged with Pd/C (50% wet, 2.32 g, 15% weight). The reaction mixture was then evacuated and backfilled with H₂ (balloon, 1 atm) and left to stir overnight. The reaction was then filtered through a celite pad and concentrated to provide [1-(5-aminopyridin-2-yl)piperidin-4-yl]methanol (5.85 g, 45% yield) as a pale yellow oil, which was used in the next step without additional purification. LCMS: m/z=208.25 (M+H)⁺. ¹H NMR (300 MHz, DMSO-d₆) δ 7.59 (dd, J=2.9, 0.7 Hz, 1H), 6.89 (dd, J=8.8, 2.9 Hz, 1H), 6.61 (dd, J=8.9, 0.8 Hz, 1H), 4.56-4.39 (m, 3H), 4.02-3.89 (m, 2H), 3.26 (dd, J=6.3, 5.3 Hz, 2H), 2.60-2.52 (m, 2H), 1.76-1.62 (m, 2H), 1.49 (s, 1H), 1.12 (dd, J=12.2, 4.0 Hz, 2H).

### Step-3: Synthesis of 3-((6-(4-(hydroxymethyl)piperidin-1-yl)pyridin-3-yl)amino)propanoic acid

A pressure vessel was charged with [1-(5-aminopyridin-2-yl)piperidin-4-yl]methanol (5.8 g, 27.9 mmol, 1 equiv) and acrylic acid (1.9 mL, 27.9 mmol, 1 equiv) dissolved in 1,4-dioxane (58 mL, 0.5 M). The reaction mixture was then stirred at 90° C. After 16 h, 25% of the starting amine was still present. Additional acrylic acid (0.25 equiv) was added and the reaction mixture was stirred at 90° C. for another 16 h. Then the mixture was concentrated, the obtained residue was diluted with EtOAc and refluxed, and the solution was then decanted. The residue (black gum) was redissolved in MeOH and concentrated to yield 3-({6-[4-(hydroxymethyl)piperidin-1-yl]pyridin-3-yl}amino)propanoic acid (6.4 g, 78% yield). LCMS: m/z=280.25 (M+H)⁺; 278.0 (M−H)⁺. ¹H NMR (300 MHz, DMSO-d₆) δ 7.59 (d, J=2.9 Hz, 1H), 6.93 (dd, J=8.9, 3.0 Hz, 1H), 6.68 (d, J=8.9 Hz, 1H), 4.00 (dt, J=12.8, 3.3 Hz, 2H), 3.39 (t, J=7.1 Hz, 1H), 3.26 (d, J=6.3 Hz, 3H), 2.63-2.53 (m, 2H), 2.47-2.29 (m, 3H), 1.67 (s, 2H), 1.13 (dd, J=12.2, 4.0 Hz, 2H).

### Step-4 and Step-5: Synthesis of (1-(5-(2,4-dioxotetrahydropyrimidin-1(2H)-yl)pyridin-2-yl)piperidin-4-yl)methyl acetate and 1-(6-(4-(hydroxymethyl)piperidin-1-yl)pyridin-3-yl)dihydropyrimidine-2,4(1H,3H)-dione

3-({6-[4-(hydroxymethyl)piperidin-1-yl]pyridin-3-yl}amino)propanoic acid (6.4 g, 21.76 mmol, 1.0 equiv), and urea (2.614 g, 43.53 mmol, 2.0 equiv) were dissolved in glacial acetic acid (64 mL, 10 vol) and left to stir at 90° C. for 48 h. The reaction mixture was then concentrated and the obtained residue was dissolved in EtOH (100 mL), followed by the addition of H₂SO₄ (0.012 mL, 0.022 mmol, 0.01 equiv) and the mixture was stirred at room temperature for 48 h. The reaction mixture pH was adjusted to 10-11 with KHSO₄, concentrated, and purified by silica gel chromatography (DCM:MeOH=9:1). The obtained material was further triturated with DCM to provide 1-{6-[4-(hydroxymethyl)piperidin-1-yl]pyridin-3-yl}-1,3-diazinane-2,4-dione (1.28 g, 18% yield). LCMS: m/z=305.05 (M+H)⁺.

¹H NMR (300 MHz, DMSO-d₆) δ 10.34 (s, 1H), 8.04 (d, J=2.7 Hz, 1H), 7.47 (dd, J=9.0, 2.8 Hz, 1H), 6.84 (d, J=9.1 Hz, 1H), 4.47 (t, J=5.3 Hz, 1H), 4.29 (d, J=13.0 Hz, 2H), 3.70 (t, J=6.7 Hz, 2H), 3.27 (t, J=5.7 Hz, 2H), 2.83-2.65 (m, 4H), 1.77-1.54 (m, 3H), 1.18-1.03 (m, 2H).

### Step-6: Synthesis of 1-(5-(2,4-dioxotetrahydropyrimidin-1(2H)-yl)pyridin-2-yl)piperidine-4-carbaldehyde

To a solution of 1-{6-[4-(hydroxymethyl)piperidin-1-yl]pyridin-3-yl}-1,3-diazinane-2,4-dione (0.715 g, 2.3 mmol, 1 equiv) dissolved in anhydrous DCM (0.3 M) was added Dess-Martin periodinane (1.073 g, 2.53 mmol, 1.1 equiv) in anhydrous DCM (0.15 M) dropwise at 0° C. The reaction mixture was then warmed and left to stir at room temperature for one hour. The reaction was monitored by TLC and LCMS. The reaction mixture was then diluted with sat. aq. Na₂S₂O₃ and the organic layer was separated and washed with sat. aq. NaHCO₃. The aqueous layers were combined and back-extracted several times with DCM. The combined organic phase was dried over Na₂SO₄ and concentrated to provide 1-[5-(2,4-dioxo-1,3-diazinan-1-yl)pyridin-2-yl]piperidine-4-carbaldehyde (0.56 g, yield 77%) as a beige solid. The product tends to form a stable hydrate. LCMS: ESI(+) m/z==305.11 [M+H]⁺. ¹H NMR (300 MHz, DMSO d₆) δ 10.35 (s, 1H), 9.63 (d, J=0.9 Hz, 1H), 8.08-8.03 (m, 1H), 7.50 (dd, J=9.0, 2.8 Hz, 1H), 6.88 (d, J=9.1 Hz, 1H), 4.13 (dt, J=13.2, 4.1 Hz, 2H), 3.70 (t, J=6.7 Hz, 2H), 3.11-2.99 (m, 2H), 2.71 (d, J=6.7 Hz, 2H), 2.66-2.55 (m, 1H), 1.90 (dd, J=13.3, 3.9 Hz, 2H), 1.55-1.42 (m, 2H).

### SFC Separation of Harnesses

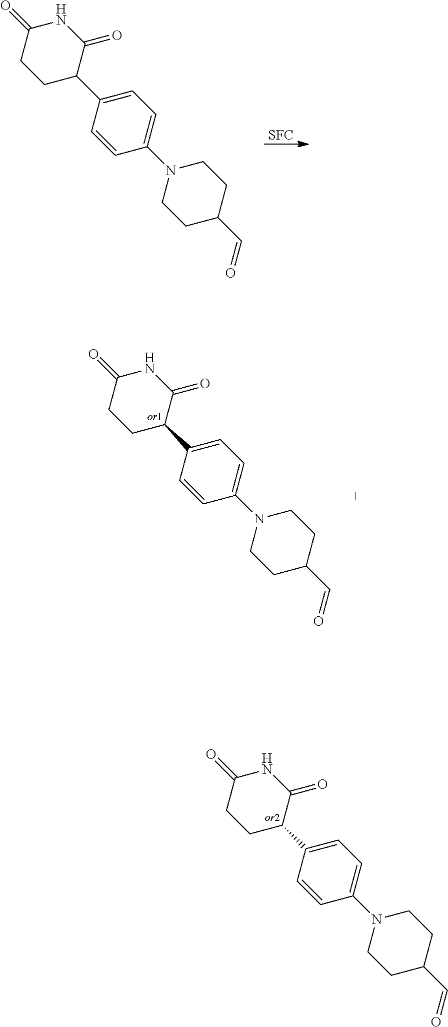 [SMILES: O=CC1CCN(c2ccc(C3CCC(=O)NC3=O)cc2)CC1 ; "SFC" ; O=CC1CCN(c2ccc([C@H]3CCC(=O)NC3=O)cc2)CC1 ; O=CC1CCN(c2ccc([C@@H]3CCC(=O)NC3=O)cc2)CC1]

### (R)-1-(4-(2,6-dioxopiperidin-3-yl)phenyl)piperidine-4-carbaldehyde and (S)-1-(4-(2,6-dioxopiperidin-3-yl)phenyl)piperidine-4-carbaldehyde (HA-XX, Peak 1 and HA-XX, Peak 2)

Racemic 1-(4-(2,6-dioxopiperidin-3-yl)phenyl)piperidine-4-carbaldehyde was purified by prep-SFC (column: DAICEL CHIRALPAK AD (250 mm×30 mm, 10 μm); mobile phase: [Neu-IPA]; B %: 65%-65%, 4.5; 650 min) to give peak 1 (13.64 g, 45.41 mmol, 48.7% yield) as yellow solid and crude peak 2. Crude peak 2 was purified by prep-SFC (column: DAICEL CHIRALPAK AD (250 mm×30 mm, 10 μm); mobile phase: [IPA-ACN]; B %: 65%-65%, 4; 580 min) to give peak 2 (8.13 g, 27.07 mmol, 29.04% yield) as an off-white solid.

HA-XX (peak 1):

LCMS: m/z=299.1 (M−H)⁻.

SFC: ee %=98.3% under 220 nm.

¹H NMR: (400 MHz, DMSO-d₆) δ 10.78 (s, 1H), 9.63 (s, 1H), 7.04 (d, J=8.4 Hz, 2H), 6.90 (d, J=8.8 Hz, 2H), 3.75-3.68 (m, 1H), 3.62-3.52 (m, 2H), 2.86-2.75 (m, 2H), 2.68-2.57 (m, 1H), 2.49-2.40 (m, 2H), 2.18-2.08 (m, 1H), 2.05-1.88 (m, 3H), 1.64-1.51 (m, 2H).

HA-XX (peak 2):

LCMS: m/z=299.1 (M−H).

SFC: ee %=100% under 220 nm.

¹H NMR (400 MHz, DMSO-d₆) δ 10.75 (s, 1H), 9.63 (s, 1H), 7.07-7.01 (m, 2H), 6.89 (d, J=8.8 Hz, 2H), 3.75-3.68 (m, 1H), 3.61-3.53 (m, 2H), 2.85-2.75 (m, 2H), 2.68-2.58 (m, 1H), 2.49-2.42 (m, 2H), 2.19-2.07 (m, 1H), 2.05-1.87 (m, 3H), 1.64-1.51 (m, 2H).

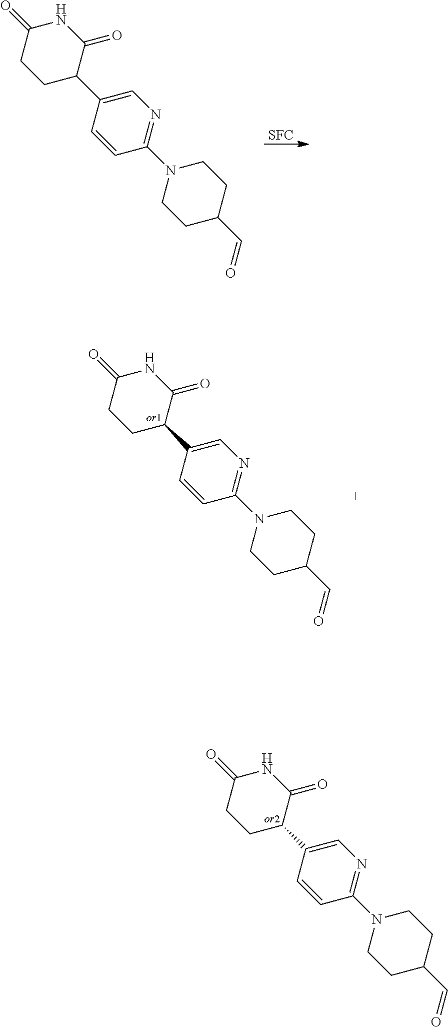 [SMILES: O=CC1CCN(c2ccc(C3CCC(=O)NC3=O)cn2)CC1 ; "SFC" ; O=CC1CCN(c2ccc([C@H]3CCC(=O)NC3=O)cn2)CC1 ; O=CC1CCN(c2ccc([C@@H]3CCC(=O)NC3=O)cn2)CC1]

### (R)-1-(5-(2,6-dioxopiperidin-3-yl)pyridin-2-yl)piperidine-4-carbaldehyde and (S)-1-(5-(2,6-dioxopiperidin-3-yl)pyridin-2-yl)piperidine-4-carbaldehyde (HA-XX, Peak 1 and HA-XX, Peak 2)

Racemic 1-(5-(2,6-dioxopiperidin-3-yl)pyridin-2-yl)piperidine-4-carbaldehyde was purified by SFC (column: REGIS (s,s) WHELK-Ol (250 mm×50 mm, 10 μm); mobile phase: [IPA-ACN]; B %: 60%-60%, B2.7; 300 min) and concentrated under vacuum to provide a residue. The residue was triturated with ethyl acetate (60.0 mL) at 20° C. for 2 h, filtered, and the filter cake was concentrated under vacuum to provide HA-XX, peak 1 (17.79 g, 58.3 mmol, 40.5% yield) and HA-XX, peak 2, HA-33 (16.05 g, 52.6 mmol, 36.5% yield) as white solids.

HA-XX (peak 1):

LCMS: m/z=320.0 (M+19)+.

¹H NMR (400 MHz, DMSO-d₆) δ 10.79 (s, 1H), 9.61 (s, 1H), 7.94 (d, J=2.4 Hz, 1H), 7.38 (dd, J=2.4, 8.8 Hz, 1H), 6.82 (d, J=8.8 Hz, 1H), 4.13-4.08 (m, 2H), 3.73-3.70 (m, 1H), 3.04-2.97 (m, 2H), 2.67-2.53 (m, 3H), 2.22-2.12 (m, 1H), 2.00-1.93 (m, 1H), 1.90-1.85 (m, 2H), 1.49-1.45 (m, 2H).

SFC: 100% ee under 220 nm.

HA-XX (peak 2):

LCMS: m/z=320.0 (M+19)+.

¹H NMR (400 MHz, DMSO-d₆) δ 10.79 (s, 1H), 9.61 (s, 1H), 7.94 (d, J=2.4 Hz, 1H), 7.38 (dd, J=2.4, 8.8 Hz, 1H), 6.82 (d, J=8.8 Hz, 1H), 4.13-4.09 (m, 2H), 3.74-3.70 (m, 1H), 3.04-2.97 (m, 2H), 2.67-2.53 (m, 3H), 2.22-2.12 (m, 1H), 2.00-1.93 (m, 1H), 1.90-1.85 (m, 2H), 1.49-1.46 (m, 2H).

SFC: 100% ee under 220 nm.

### General Procedures for Synthesis of Final Exemplary Chimeric Targeting Molecules (CTMs)

### Reductive Amination Example

### Synthesis of rac-N-[3-(2-tert-butyl-5-{2-[(4-{1-[(1-{4-[(3R)-2,6-dioxopiperidin-3-yl]phenyl}piperidin-4-yl)methyl]piperidin-4-yl}phenyl)amino]pyrimidin-4-yl}-1,3-thiazol-4-yl)-2-fluorophenyl]propane-1-sulfonamide

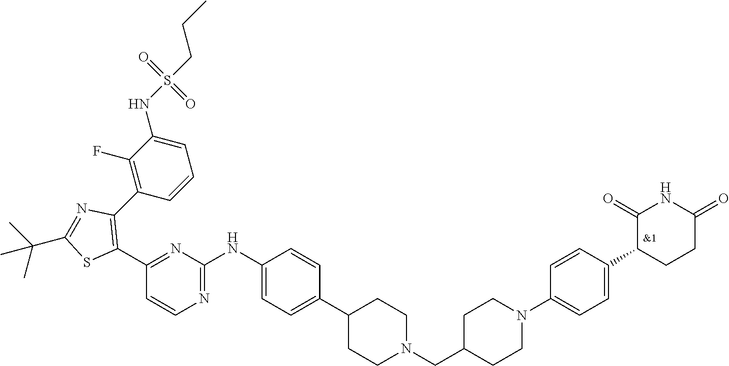 [SMILES: CCCS(=O)(=O)Nc1cccc(-c2nc(C(C)(C)C)sc2-c2ccnc(Nc3ccc(C4CCN(CC5CCN(c6ccc([C@H]7CCC(=O)NC7=O)cc6)CC5)CC4)cc3)n2)c1F]

N-{3-[2-tert-butyl-5-(2-{[4-(piperidin-4-yl)phenyl]amino}pyrimidin-4-yl)-1,3-thiazol-4-yl]-2-fluorophenyl}propane-1-sulfonamide (93 mg, 0.153 mmol) and rac-1-{4-[(3R)-2,6-dioxopiperidin-3-yl]phenyl}piperidine-4-carbaldehyde (57 mg, 1.25 equiv) were combined in DCE (0.1 M) and DIEA (0.08 mL, 3 equiv) and the reaction mixture was stirred for fifteen minutes, followed by the addition of STAB (89 mg, 2.75 equiv). The reaction was then stirred for 4 h, followed by concentration by rotary evaporator. The reaction was then diluted with DMF (0.5 mL), filtered through a syringe filter, and purified by HPLC to provide the desired product (32 mg, 22%). LCMS: C₄₈H₅₇FN₈O₄S₂ requires: 892.4, found: m/z=893.3 [M+H]⁺.

### Amide Coupling Examples

### Synthesis of rac-N-[3-(2-tert-butyl-5-{2-[(4-{1-[2-({2-[(3R)-2,6-dioxopiperidin-3-yl]-1,3-dioxoisoindol-5-yl}oxy)acetyl]piperidin-4-yl}phenyl)amino]pyrimidin-4-yl}-1,3-thiazol-4-yl)-2-fluorophenyl]propane-1-sulfonamide

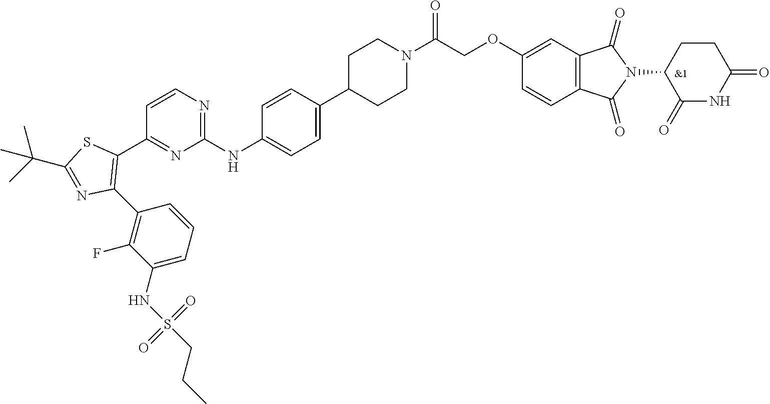 [SMILES: CCCS(=O)(=O)Nc1cccc(-c2nc(C(C)(C)C)sc2-c2ccnc(Nc3ccc(C4CCN(C(=O)COc5ccc6c(c5)C(=O)N([C@@H]5CCC(=O)NC5=O)C6=O)CC4)cc3)n2)c1F]

N-{3-[2-tert-butyl-5-(2-{[4-(piperidin-4-yl)phenyl]amino}pyrimidin-4-yl)-1,3-thiazol-4-yl]-2-fluorophenyl}propane-1-sulfonamide (20 mg, 0.033 mmol), rac-({2-[(3R)-2,6-dioxopiperidin-3-yl]-1,3-dioxoisoindol-5-yl}oxy)acetic acid (12 mg, 1.1 equiv) and HATU (13 mg, 1.1 equiv) in DMF (0.2 M) and DIEA (0.023 mL, 4 equiv) was stirred at rt for 4 h, followed by filtration by syringe filter and purification by HPLC to provide the desired product (13 mg, 39%). LCMS: C₄₆H₄₇FN₈O₈S₂ requires: 922.3, found: m/z=923.5 [M+H]⁺.

### Synthesis of rac-N-{3-[1-(4-{4-[2-(1-{5-[(3R)-2,6-dioxopiperidin-3-yl]pyridin-2-yl}piperidin-4-yl)acetyl]piperazine-1-carbonyl}phenyl)-3-(pyridin-4-yl)pyrazol-4-yl]-2-fluorophenyl}propane-1-sulfonamide

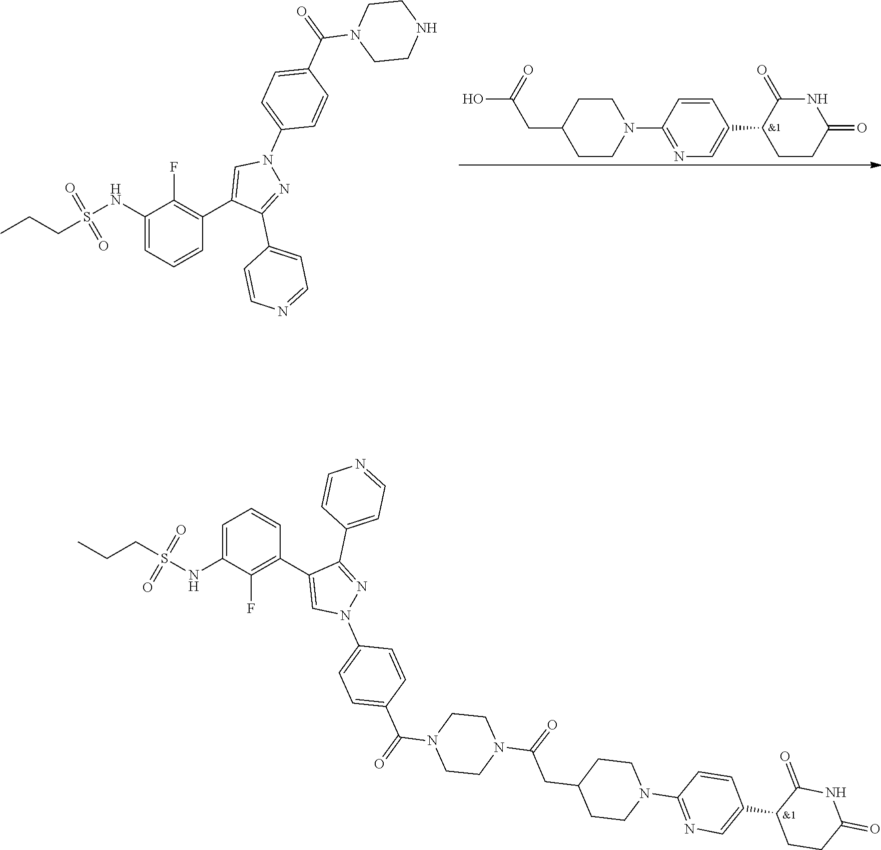 [SMILES: CCCS(=O)(=O)Nc1cccc(-c2cn(-c3ccc(C(=O)N4CCNCC4)cc3)nc2-c2ccncc2)c1F ; O=C(O)CC1CCN(c2ccc([C@H]3CCC(=O)NC3=O)cn2)CC1 ; CCCS(=O)(=O)Nc1cccc(-c2cn(-c3ccc(C(=O)N4CCN(C(=O)CC5CCN(c6ccc([C@H]7CCC(=O)NC7=O)cn6)CC5)CC4)cc3)nc2-c2ccncc2)c1F]

N-(2-fluoro-3-{1-[4-(piperazine-1-carbonyl)phenyl]-3-(pyridin-4-yl)pyrazol-4-yl}phenyl)propane-1-sulfonamide (20 mg, 0.0336 mmol), rac-(1-{5-[(3R)-2,6-dioxopiperidin-3-yl]pyridin-2-yl}piperidin-4-yl)acetic acid (12 mg, 1.1 equiv), and HATU (13 mg, 1.1 equiv) in DMF (0.2 M) and DIEA (0.023 mL, 4 equiv) was stirred at rt for 4 h. Filtration by syringe filter and purification by HPLC provided the desired product (23 mg, 66%). LCMS: C₄₅H₄₈FN₉O₆S requires: 861.3, found: m/z=862.2 [M+H]⁺.

### Synthesis of rac-N-[3-(1-{1-[1-(1-{5-[(3R)-2,6-dioxopiperidin-3-yl]pyridin-2-yl}piperidine-4-carbonyl)piperidine-4-carbonyl]piperidin-4-yl}-3-(pyridin-4-yl)pyrazol-4-yl)-2-fluorophenyl]propane-1-sulfonamide

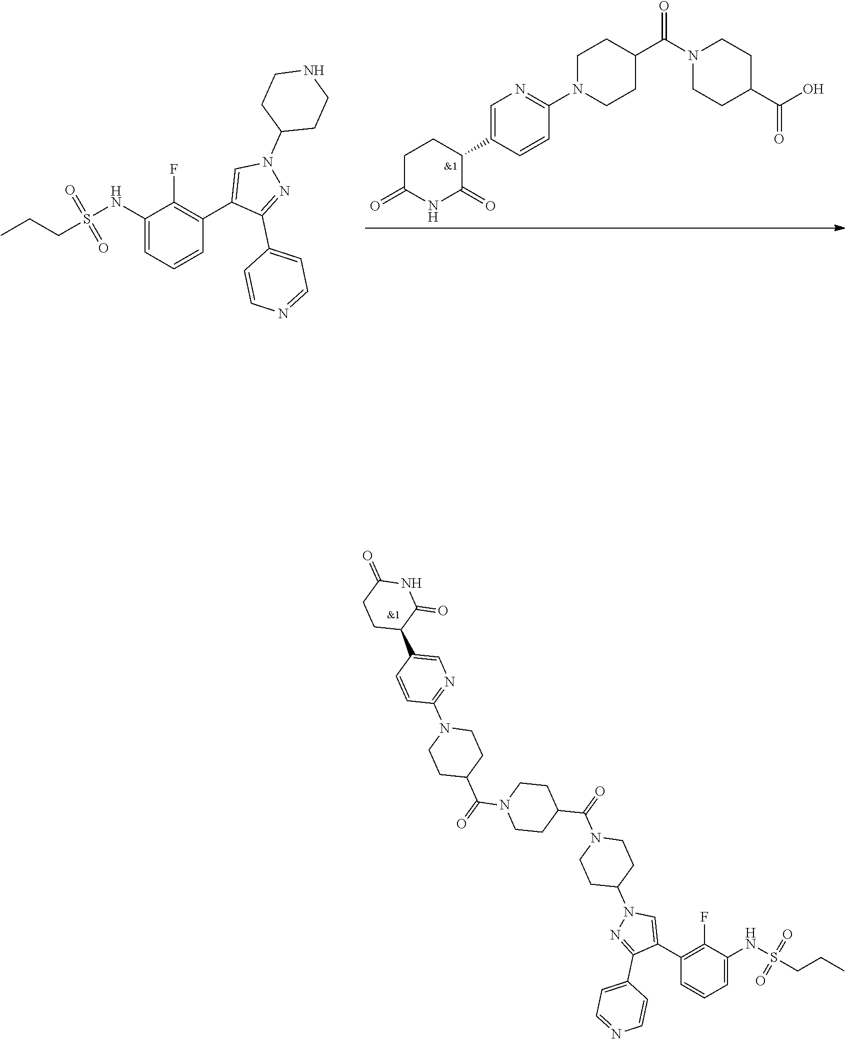 [SMILES: CCCS(=O)(=O)Nc1cccc(-c2cn(C3CCNCC3)nc2-c2ccncc2)c1F ; O=C1CC[C@H](c2ccc(N3CCC(C(=O)N4CCC(C(=O)O)CC4)CC3)nc2)C(=O)N1 ; CCCS(=O)(=O)Nc1cccc(-c2cn(C3CCN(C(=O)C4CCN(C(=O)C5CCN(c6ccc([C@H]7CCC(=O)NC7=O)cn6)CC5)CC4)CC3)nc2-c2ccncc2)c1F]

N-{2-fluoro-3-[1-(piperidin-4-yl)-3-(pyridin-4-yl)pyrazol-4-yl]phenyl}propane-1-sulfonamide (10 mg, 0.023 mmol), rac-1-(1-{5-[(3R)-2,6-dioxopiperidin-3-yl]pyridin-2-yl}piperidine-4-carbonyl)piperidine-4-carboxylic acid (10 mg, 1.03 equiv), and HATU (9 mg, 1.05 equiv) in DMF (0.1 M) and DIEA (0.02 mL, 5 equiv) was stirred at rt for 4 h. Filtration by syringe filter and purification by HPLC provided the desired product (5.4 mg, 27%). LCMS: C₄₄H₅₂FN₉O₆S requires: 853.4, found: m/z=854.7 [M+H]⁺.

### N-{3-[1-(4-{4-[(1-{5-[(3S)-2,6-dioxopiperidin-3-yl]pyridin-2-yl}piperidin-4-yl)methyl]piperazin-1-yl}phenyl)-3-(pyridin-4-yl)-1H-pyrazol-4-yl]-2-fluorophenyl}propane-1-sulfonamide (1)

LCMS: C₄₃H₄₈FN₉O₄S requires: 805.4, found: m/z=806.2 [M+H]⁺.

### N-{3-[1-(4-{4-[(1-{5-[(3R)-2,6-dioxopiperidin-3-yl]pyridin-2-yl}piperidin-4-yl)methyl]piperazin-1-yl}phenyl)-3-(pyridin-4-yl)-1H-pyrazol-4-yl]-2-fluorophenyl}propane-1-sulfonamide (2)

¹H NMR (500 MHz, DMSO-d₆) δ 10.88 (s, 1H), 9.69 (s, 1H), 9.50 (s, 1H), 8.77 (s, 1H), 8.65 (d, J=5.4 Hz, 2H), 7.94 (d, J=2.4 Hz, 1H), 7.87 (d, J=8.8 Hz, 2H), 7.63 (d, J=5.7 Hz, 3H), 7.49 (td, J=7.7, 1.9 Hz, 1H), 7.40-7.34 (m, 1H), 7.31 (t, J=7.8 Hz, 1H), 7.25-7.17 (m, 2H), 4.28 (d, J=13.0 Hz, 2H), 3.94 (d, J=10.9 Hz, 3H), 3.89-3.79 (m, 1H), 3.25-3.10 (m, 6H), 3.09-2.94 (m, 4H), 2.75-2.60 (m, 1H), 2.32-2.13 (m, 2H), 2.03-1.92 (m, 1H), 1.88 (d, J=12.9 Hz, 2H), 1.75-1.60 (m, 2H), 1.28 (d, J=11.4 Hz, 1H), 0.92 (t, J=7.4 Hz, 3H).

LCMS: C₄₃H₄₈FN₉O₄S requires: 805.4, found: m/z=806.3 [M+H]⁺.

### Rac-N-{3-[1-(6-{4-[(1-{5-[(3R)-2,6-dioxopiperidin-3-yl]pyridin-2-yl}piperidin-4-yl)methyl]piperazin-1-yl}pyridin-3-yl)-3-(pyridin-4-yl)-1H-pyrazol-4-yl]-2-fluorophenyl}propane-1-sulfonamide (3)

LCMS: C₄₂H₄₇FN₁₀O₄S requires: 806.3, found: m/z=807.2 [M+H]⁺.

### N-{3-[1-(6-{4-[(1-{4-[(3S)-2,6-dioxopiperidin-3-yl]phenyl}piperidin-4-yl)methyl]piperazin-1-yl}pyridin-3-yl)-3-(pyridin-4-yl)-1H-pyrazol-4-yl]-2-fluorophenyl}propane-1-sulfonamide (4)

LCMS: C₄₃H₄₈FN₉O₄S requires: 805.4, found: m/z=806.2 [M+H]⁺.

### Rac-N-{3-[1-(4-{[1-(1-{5-[(3R)-2,6-dioxopiperidin-3-yl]pyridin-2-yl}piperidine-4-carbonyl)piperidin-4-yl]oxy}phenyl)-3-(pyridin-4-yl)-1H-pyrazol-4-yl]-2-fluorophenyl}propane-1-sulfonamide (5)

LCMS: C₄₄H₄₇FN₈O₆S requires: 834.3, found: m/z=835.2 [M+H]⁺.

### Rac-N-{3-[1-(1-{1-[(1-{5-[(3R)-2,6-dioxopiperidin-3-yl]pyridin-2-yl}piperidin-4-yl)methyl]piperidine-4-carbonyl}piperidin-4-yl)-3-(pyridin-4-yl)-1H-pyrazol-4-yl]-2-fluorophenyl}propane-1-sulfonamide (6)

LCMS: C₄₄H₅₄FN₉O₅S requires: 839.4, found: m/z=840.3 [M+H]⁺.

### Rac-N-[3-(1-{1-[1-(1-{5-[(3R)-2,6-dioxopiperidin-3-yl]pyridin-2-yl}piperidine-4-carbonyl)piperidine-4-carbonyl]piperidin-4-yl}-3-(pyridin-4-yl)-1H-pyrazol-4-yl)-2-fluorophenyl]propane-1-sulfonamide (7)

LCMS: C₄₄H₅₂FN₉O₆S requires: 853.4, found: m/z=854.7 [M+H]⁺.

### Rac-N-[3-(1-{1-[1-(1-{5-[(3R)-2,6-dioxopiperidin-3-yl]pyridin-2-yl}piperidine-4-carbonyl)-4-methylpiperidine-4-carbonyl]piperidin-4-yl}-3-(pyridin-4-yl)-1H-pyrazol-4-yl)-2-fluorophenyl]propane-1-sulfonamide (8)

LCMS: C₄₅H₅₄FN₉O₆S requires: 867.4, found: m/z=868.2 [M+H]⁺.

### Rac-N-[3-(1-{1-[1-(1-{4-[(3R)-2,6-dioxopiperidin-3-yl]phenyl}piperidine-4-carbonyl)piperidine-4-carbonyl]piperidin-4-yl}-3-(pyridin-4-yl)-1H-pyrazol-4-yl)-2-fluorophenyl]propane-1-sulfonamide (9)

LCMS: C₄₅H₅₃FN₈O₆S requires: 852.4, found: m/z=853.2 [M+H]⁺.

### N-{3-[1-(4-{4-[(1-{4-[(3S)-2,6-dioxopiperidin-3-yl]phenyl}piperidin-4-yl)methyl]piperazin-1-yl}phenyl)-3-(pyridin-4-yl)-1H-pyrazol-4-yl]-2-fluorophenyl}propane-1-sulfonamide (10)

¹H NMR (500 MHz, DMSO-d₆) δ 10.78 (s, 1H), 9.68 (s, 1H), 8.71 (s, 1H), 8.55 (d, J=5.2 Hz, 2H), 7.83 (s, 2H), 7.45 (dd, J=16.5, 6.4 Hz, 3H), 7.32 (dt, J=26.2, 7.4 Hz, 2H), 7.24-7.00 (m, 4H), 6.92 (d, J=8.2 Hz, 2H), 3.73 (dd, J=11.1, 5.0 Hz, 1H), 3.21 (s, 6H), 3.02 (dd, J=9.1, 6.3 Hz, 2H), 2.65 (t, J=15.4 Hz, 3H), 2.32-2.07 (m, 1H), 2.02 (dd, J=13.0, 5.6 Hz, 1H), 1.85 (d, J=12.4 Hz, 2H), 1.69 (q, J=7.6 Hz, 2H), 0.92 (t, J=7.4 Hz, 3H).

LCMS: C₄₄H₄₉FN₈O₄S requires: 804.4, found: m/z=805.7 [M+H]⁺.

### N-{3-[1-(4-{4-[(1-{4-[(3R)-2,6-dioxopiperidin-3-yl]phenyl}piperidin-4-yl)methyl]piperazine-1-carbonyl}phenyl)-3-(pyridin-4-yl)-1H-pyrazol-4-yl]-2-fluorophenyl}propane-1-sulfonamide (11)

LCMS: C₄₅H₄₉FN₈O₅S requires: 832.4, found: m/z=833.2 [M+H]⁺.

### N-{3-[1-(4-{4-[(1-{4-[(3S)-2,6-dioxopiperidin-3-yl]phenyl}piperidin-4-yl)methyl]piperazine-1-carbonyl}phenyl)-3-(pyridin-4-yl)-1H-pyrazol-4-yl]-2-fluorophenyl}propane-1-sulfonamide (12)

LCMS: C₄₅H₄₉FN₈O₅S requires: 832.4, found: m/z=833.3 [M+H]⁺.

### Rac-N-{3-[1-(4-{4-[2-(1-{5-[(3R)-2,6-dioxopiperidin-3-yl]pyridin-2-yl}piperidin-4-yl)acetyl]piperazine-1-carbonyl}phenyl)-3-(pyridin-4-yl)-1H-pyrazol-4-yl]-2-fluorophenyl}propane-1-sulfonamide (13)

LCMS: C₄₅H₄₈FN₉O₆S requires: 861.3, found: m/z=862.2 [M+H]⁺.

### Rac-N-[3-(1-{4-[4-(1-{4-[(3R)-2,6-dioxopiperidin-3-yl]phenyl}piperidine-4-carbonyl)piperazine-1-carbonyl]phenyl}-3-(pyridin-4-yl)-1H-pyrazol-4-yl)-2-fluorophenyl]propane-1-sulfonamide (14)

LCMS: C₄₅H₄₇FN₈O₆S requires: 846.3, found: m/z=847.2 [M+H]⁺.

### N-{3-[1-(4-{1-[(1-{4-[(3S)-2,6-dioxopiperidin-3-yl]phenyl}piperidin-4-yl)methyl]piperidin-4-yl}phenyl)-3-(pyridin-4-yl)-1H-pyrazol-4-yl]-2-fluorophenyl}propane-1-sulfonamide (15)

LCMS: C₄₅H₅₀FN₇O₄S requires: 803.4, found: m/z=804.2 [M+H]⁺.

### N-{3-[1-(4-{4-[(1-{4-[(3R)-2,6-dioxopiperidin-3-yl]phenyl}piperidin-4-yl)methyl]piperazin-1-yl}phenyl)-3-(pyridin-4-yl)-1H-pyrazol-4-yl]-2-fluorophenyl}propane-1-sulfonamide (16)

¹H NMR (500 MHz, DMSO-d₆) δ 10.78 (s, 1H), 9.69 (s, 1H), 8.69 (d, J=3.0 Hz, 1H), 8.57-8.52 (m, 2H), 8.15 (s, 1H), 7.79 (d, J=8.7 Hz, 2H), 7.50-7.44 (m, 1H), 7.47-7.41 (m, 2H), 7.37-7.31 (m, 1H), 7.29 (t, J=7.8 Hz, 1H), 7.13-7.08 (m, 2H), 7.05 (d, J=8.2 Hz, 2H), 6.90 (d, J=8.3 Hz, 2H), 5.77 (s, 1H), 3.73 (dd, J=10.9, 4.9 Hz, 1H), 3.68 (d, J=11.8 Hz, 2H), 3.24 (d, J=4.8 Hz, 3H), 3.02 (dd, J=8.7, 6.6 Hz, 2H), 2.67 (d, J=11.7 Hz, 2H), 2.25 (d, J=7.1 Hz, 2H), 2.18-2.07 (m, 1H), 2.02 (dt, J=13.3, 4.9 Hz, 1H), 1.83 (d, J=12.5 Hz, 2H), 1.74-1.64 (m, 3H), 1.30-1.19 (m, 2H), 0.91 (t, J=7.4 Hz, 3H).

LCMS: C₄₄H₄₉FN₈O₄S requires: 804.4, found: m/z=805.2 [M+H]⁺.

### Rac-N-{3-[1-(4-{4-[(1-{5-[(3R)-2,6-dioxopiperidin-3-yl]pyridin-2-yl}-4-methylpiperidin-4-yl)methyl]piperazin-1-yl}phenyl)-3-(pyridin-4-yl)-1H-pyrazol-4-yl]-2-fluorophenyl}propane-1-sulfonamide (17)

LCMS: C₄₄H₅₀FN₉O₄S requires: 819.4, found: m/z=820.2 [M+H]⁺.

### Rac-N-{3-[1-(4-{4-[(1-{4-[(3R)-2,6-dioxopiperidin-3-yl]-2-fluorophenyl}piperidin-4-yl)methyl]piperazin-1-yl}phenyl)-3-(pyridin-4-yl)-1H-pyrazol-4-yl]-2-fluorophenyl}propane-1-sulfonamide (18)

¹H NMR (500 MHz, DMSO-d₆) δ 10.83 (s, 1H), 9.70 (s, 1H), 9.43 (s, 1H), 8.80 (s, 1H), 8.69 (d, J=5.6 Hz, 2H), 7.89 (d, J=8.7 Hz, 2H), 7.69 (d, J=5.5 Hz, 2H), 7.50 (td, J=7.7, 1.9 Hz, 1H), 7.41-7.36 (m, 1H), 7.32 (t, J=7.8 Hz, 1H), 7.22 (d, J=8.8 Hz, 2H), 7.08-6.95 (m, 3H), 3.95 (d, J=11.6 Hz, 2H), 3.82 (dd, J=11.8, 4.9 Hz, 1H), 3.73-3.62 (m, 2H), 3.40 (d, J=11.5 Hz, 2H), 3.30-3.11 (m, 6H), 3.10-3.01 (m, 2H), 2.78-2.60 (m, 3H), 2.21 (dd, J=12.4, 4.2 Hz, 1H), 2.06-1.95 (m, 2H), 1.95-1.84 (m, 2H), 1.70 (q, J=7.6 Hz, 2H), 1.56-1.36 (m, 2H), 0.93 (t, J=7.4 Hz, 3H).

LCMS: C₄₄H₄₈F₂N₈O₄S requires: 822.3, found: m/z=823.2 [M+H]⁺.

### Rac-N-{3-[1-(4-{4-[(1-{4-[(3R)-2,6-dioxopiperidin-3-yl]-3-fluorophenyl}piperidin-4-yl)methyl]piperazin-1-yl}phenyl)-3-(pyridin-4-yl)-1H-pyrazol-4-yl]-2-fluorophenyl}propane-1-sulfonamide (19)

LCMS: C₄₄H₄₈F₂N₈O₄S requires: 822.4, found: m/z=823.2 [M+H]⁺.

### Rac-N-{3-[1-(4-{4-[(1-{5-[(3R)-2,6-dioxopiperidin-3-yl]pyridin-2-yl}piperidin-4-yl)methyl]piperazin-1-yl}phenyl)-3-(pyridin-4-yl)-1H-pyrazol-4-yl]-2-fluorophenyl}propane-1-sulfonamide (20)

¹H NMR (500 MHz, DMSO-d₆) δ 10.90 (s, 1H), 9.70 (s, 1H), 9.53 (s, 1H), 8.80 (s, 1H), 8.68 (d, J=5.4 Hz, 2H), 7.95 (d, J=2.3 Hz, 1H), 7.89 (d, J=8.6 Hz, 2H), 7.67 (d, J=5.5 Hz, 3H), 7.55-7.46 (m, 1H), 7.35 (dt, J=29.6, 7.5 Hz, 2H), 7.22 (d, J=8.7 Hz, 2H), 7.16 (s, 1H), 4.28 (d, J=13.1 Hz, 2H), 3.95 (d, J=11.1 Hz, 2H), 3.86 (dd, J=12.6, 4.9 Hz, 1H), 3.67 (d, J=10.4 Hz, 2H), 3.31-3.10 (m, 6H), 3.11-2.94 (m, 3H), 2.77-2.63 (m, 1H), 2.25 (ddd, J=19.8, 14.4, 7.6 Hz, 2H), 1.98 (dq, J=13.1, 4.8, 4.3 Hz, 1H), 1.89 (d, J=12.8 Hz, 2H), 1.70 (q, J=7.6 Hz, 2H), 1.30 (q, J=12.9, 12.3 Hz, 2H), 0.93 (t, J=7.4 Hz, 3H).

LCMS: C₄₃H₄₈FN₉O₄S requires: 805.4, found: m/z=806.2 [M+H]⁺.

### Rac-N-{3-[1-(4-{4-[(1-{4-[(3R)-2,6-dioxopiperidin-3-yl]phenyl}piperidin-4-yl)methyl]piperazin-1-yl}phenyl)-3-(pyridin-4-yl)-1H-pyrazol-4-yl]-2-fluorophenyl}propane-1-sulfonamide (21)

LCMS: C₄₄H₄₉FN₈O₄S requires: 804.4, found: m/z=805.2 [M+H]⁺.

### Rac-N-{3-[1-(4-{1-[(1-{4-[(3R)-2,6-dioxopiperidin-3-yl]-2-fluorophenyl}piperidin-4-yl)methyl]piperidin-4-yl}phenyl)-3-(pyridin-4-yl)-1H-pyrazol-4-yl]-2-fluorophenyl}propane-1-sulfonamide (22)

LCMS: C₄₅H₄₉F₂N₇O₄S requires: 821.4, found: m/z=822.2 [M+H]⁺.

### Rac-N-{3-[1-(4-{1-[(1-{4-[(3R)-2,6-dioxopiperidin-3-yl]-3-fluorophenyl}piperidin-4-yl)methyl]piperidin-4-yl}phenyl)-3-(pyridin-4-yl)-1H-pyrazol-4-yl]-2-fluorophenyl}propane-1-sulfonamide (23)

LCMS: C₄₅H₄₉F₂N₇O₄S requires: 821.4, found: m/z=822.2 [M+H]⁺.

### Rac-N-{3-[1-(4-{1-[(1-{5-[(3R)-2,6-dioxopiperidin-3-yl]pyridin-2-yl}piperidin-4-yl)methyl]piperidin-4-yl}phenyl)-3-(pyridin-4-yl)-1H-pyrazol-4-yl]-2-fluorophenyl}propane-1-sulfonamide (24)

LCMS: C₄₄H₄₉FN₈O₄S requires: 804.4, found: m/z=805.2 [M+H]⁺.

### Rac-N-{3-[1-(4-{1-[(1-{4-[(3R)-2,6-dioxopiperidin-3-yl]phenyl}piperidin-4-yl)methyl]piperidin-4-yl}phenyl)-3-(pyridin-4-yl)-1H-pyrazol-4-yl]-2-fluorophenyl}propane-1-sulfonamide (25)

LCMS: C₄₅H₅₀FN₇O₄S requires: 803.4, found: m/z=804.3 [M+H]⁺.

### Rac-N-[3-(1-{4-[4-(1-{4-[(3R)-2,6-dioxopiperidin-3-yl]phenyl}piperidine-4-carbonyl)piperazin-1-yl]phenyl}-3-(pyridin-4-yl)-1H-pyrazol-4-yl)-2-fluorophenyl]propane-1-sulfonamide (26)

LCMS: C₄₄H₄₇FN₈O₅S requires: 818.3, found: m/z=819.2 [M+H]⁺.

### (3R)-3-{6-[4-({4-[6-({4-[4-(3-{[ethyl(methyl)sulfamoyl]amino}-2-fluorophenyl)-2-methyl-1,3-thiazol-5-yl]pyrimidin-2-yl}amino)pyridin-3-yl]piperazin-1-yl}methyl)piperidin-1-yl]pyridin-3-yl}piperidine-2,6-dione (27)

LCMS: C₄₂H₄₉FN₁₂O₄S₂ requires: 868.3, found: m/z=869.2 [M+H]⁺.

### Rac-(3R)-3-[2-(2-{4-[4-({4-[2-tert-butyl-4-(3-{[ethyl(methyl)sulfamoyl]amino}-2-fluorophenyl)-1,3-thiazol-5-yl]pyrimidin-2-yl}amino)-2-fluorophenyl]piperazin-1-yl}-2-oxoethyl)-1,2,3,4-tetrahydroisoquinolin-6-yl]piperidine-2,6-dione (28)

LCMS: C₄₆H₅₂F₂N₁₀O₅S₂ requires: 926.4, found: m/z=927.2 [M+H]⁺.

### (3R)-3-{6-[4-({4-[6-({4-[2-tert-butyl-4-(3-{[ethyl(methyl)sulfamoyl]amino}-2-fluorophenyl)-1,3-thiazol-5-yl]pyrimidin-2-yl}amino)pyridin-3-yl]piperazin-1-yl}methyl)piperidin-1-yl]pyridin-3-yl}piperidine-2,6-dione (29)

¹H NMR (500 MHz, DMSO-d₆) δ 10.81 (s, 1H), 9.70 (s, 1H), 9.56 (s, 1H), 8.35 (d, J=5.2 Hz, 1H), 7.99 (d, J=2.9 Hz, 1H), 7.95 (d, J=2.4 Hz, 1H), 7.83 (d, J=9.0 Hz, 1H), 7.55 (t, J=7.8 Hz, 1H), 7.44-7.25 (m, 4H), 6.80 (d, J=8.8 Hz, 1H), 6.44 (d, J=5.2 Hz, 1H), 4.28 (d, J=12.7 Hz, 2H), 3.73 (dd, J=12.2, 4.9 Hz, 1H), 3.14 (s, 4H), 3.06 (q, J=7.1 Hz, 2H), 2.80 (t, J=12.4 Hz, 2H), 2.75-2.61 (m, 5H), 2.40-2.34 (m, 1H), 2.31-2.12 (m, 3H), 2.05-1.92 (m, 1H), 1.80 (d, J=12.7 Hz, 3H), 1.48 (s, 10H), 1.25 (s, 2H), 1.19-1.05 (m, 2H), 1.00 (t, J=7.1 Hz, 3H).

LCMS: C₄₅H₅₅FN₁₂O₄S₂ requires: 910.4, found: m/z=911.2 [M+H]⁺.

### Rac-(3R)-3-{6-[6-({4-[6-({4-[2-tert-butyl-4-(3-{[ethyl(methyl)sulfamoyl]amino}-2-fluorophenyl)-1,3-thiazol-5-yl]pyrimidin-2-yl}amino)pyridin-3-yl]piperazin-1-yl}methyl)-2-azaspiro[3.3]heptan-2-yl]pyridin-3-yl}piperidine-2,6-dione (30)

LCMS: C₄₆H₅₅FN₁₂O₄S₂ requires: 922.4, found: m/z=923.2 [M+H]⁺.

### Rac-N-[3-(5-{2-[(4-{4-[(1-{4-[(3R)-2,6-dioxopiperidin-3-yl]phenyl}-4-fluoropiperidin-4-yl)methyl]piperazin-1-yl}phenyl)amino]pyrimidin-4-yl}-2-methyl-1,3-thiazol-4-yl)-2-fluorophenyl]propane-1-sulfonamide (31)

LCMS: C₄₄H₄₉F₂N₉O₄S₂ requires: 869.3, found: m/z=870.3 [M+H]⁺.

### Rac-N-[3-(5-{2-[(4-{4-[(1-{5-[(3R)-2,6-dioxopiperidin-3-yl]pyridin-2-yl}-4-methylpiperidin-4-yl)methyl]piperazin-1-yl}phenyl)amino]pyrimidin-4-yl}-2-methyl-1,3-thiazol-4-yl)-2-fluorophenyl]propane-1-sulfonamide (32)

LCMS: C₄₄H₅₁FN₁₀O₄S₂ requires: 866.4, found: m/z=867.3 [M+H]⁺.

### Rac-(3S)-3-{4-[4-({4-[6-({4-[4-(3-{[ethyl(methyl)sulfamoyl]amino}-2-fluorophenyl)-2-methyl-1,3-thiazol-5-yl]pyrimidin-2-yl}amino)pyridin-3-yl]piperazin-1-yl}methyl)-4-fluoropiperidin-1-yl]phenyl}piperidine-2,6-dione (33)

LCMS: C₄₃H₄₉F₂N₁₁O₄S₂ requires: 885.3, found: m/z=886.2 [M+H]⁺.

### Rac-(3S)-3-{6-[4-({4-[6-({4-[4-(3-{[ethyl(methyl)sulfamoyl]amino}-2-fluorophenyl)-2-methyl-1,3-thiazol-5-yl]pyrimidin-2-yl}amino)pyridin-3-yl]piperazin-1-yl}methyl)-4-methylpiperidin-1-yl]pyridin-3-yl}piperidine-2,6-dione (34)

LCMS: C₄₃H₅₁FN₁₂O₄S₂ requires: 882.4, found: m/z=883.2 [M+H]⁺.

### Rac-N-[3-(2-tert-butyl-5-{2-[(4-{1-[(1-{5-[(3R)-2,6-dioxopiperidin-3-yl]pyridin-2-yl}piperidin-4-yl)methyl]piperidin-4-yl}phenyl)amino]pyrimidin-4-yl}-1,3-thiazol-4-yl)-2-fluorophenyl]propane-1-sulfonamide (35)

LCMS: C₄₇H₅₆FN₉O₄S₂ requires: 893.4, found: m/z=894.2 [M+H]⁺.

### Rac-(3S)-3-[4-(4-{4-[6-({4-[2-tert-butyl-4-(3-{[ethyl(methyl)sulfamoyl]amino}-2-fluorophenyl)-1,3-thiazol-5-yl]pyrimidin-2-yl}amino)pyridin-3-yl]piperazine-1-carbonyl}piperidin-1-yl)phenyl]piperidine-2,6-dione (36)

LCMS: C₄₆H₅₄FN₁₁O₅S₂ requires: 923.4, found: m/z=924.2 [M+H]⁺.

### Rac-N-(3-{2-tert-butyl-5-[2-({4-[1-(1-{4-[(3R)-2,6-dioxopiperidin-3-yl]phenyl}piperidine-4-carbonyl)piperidin-4-yl]phenyl}amino)pyrimidin-4-yl]-1,3-thiazol-4-yl}-2-fluorophenyl)propane-1-sulfonamide (37)

LCMS: C₄₈H₅₅FN₈O₅S₂ requires: 906.4, found: m/z=907.2 [M+H]⁺.

### Rac-N-(3-{2-tert-butyl-5-[2-({4-[4-(1-{4-[(3R)-2,6-dioxopiperidin-3-yl]phenyl}piperidine-4-carbonyl)piperazin-1-yl]phenyl}amino)pyrimidin-4-yl]-1,3-thiazol-4-yl}-2-fluorophenyl)propane-1-sulfonamide (38)

LCMS: C₄₇H₅₄FN₉O₅S₂ requires: 907.4, found: m/z=908.7 [M+H]⁺.

### Rac-(3R)-3-{6-[4-(2-{4-[6-({4-[4-(3-{[ethyl(methyl)sulfamoyl]amino}-2-fluorophenyl)-2-methyl-1,3-thiazol-5-yl]pyrimidin-2-yl}amino)pyridin-3-yl]piperazin-1-yl}-2-oxoethyl)piperidin-1-yl]pyridin-3-yl}piperidine-2,6-dione (39)

LCMS: C₄₃H₄₉FN₁₂O₅S₂ requires: 896.3, found: m/z=897.2 [M+H]⁺.

### Rac-(3S)-3-[6-(4-{4-[6-({4-[4-(3-{[ethyl(methyl)sulfamoyl]amino}-2-fluorophenyl)-2-methyl-1,3-thiazol-5-yl]pyrimidin-2-yl}amino)pyridin-3-yl]piperazine-1-carbonyl}piperidin-1-yl)pyridin-3-yl]piperidine-2,6-dione (40)

LCMS: C₄₃H₄₇FN₁₂O₅S₂ requires: 882.3, found: m/z=883.2 [M+H]⁺.

### (3R)-3-{4-[4-({4-[6-({4-[4-(3-{[ethyl(methyl)sulfamoyl]amino}-2-fluorophenyl)-2-methyl-1,3-thiazol-5-yl]pyrimidin-2-yl}amino)pyridin-3-yl]piperazin-1-yl}methyl)piperidin-1-yl]phenyl}piperidine-2,6-dione (41)

LCMS: C₄₃H₅₀FN₁₁O₄S₂ requires: 867.3, found: m/z=868.2 [M+H]⁺.

### Rac-(3S)-3-[6-(4-{4-[6-({4-[2-tert-butyl-4-(3-{[ethyl(methyl)sulfamoyl]amino}-2-fluorophenyl)-1,3-thiazol-5-yl]pyrimidin-2-yl}amino)pyridin-3-yl]piperazine-1-carbonyl}-4-methylpiperidin-1-yl)pyridin-3-yl]piperidine-2,6-dione (42)

LCMS: C₄₆H₅₅FN₁₂O₅S₂ requires: 938.4, found: m/z=939.0 [M+H]⁺.

### Rac-(3S)-3-{6-[4-({4-[6-({4-[4-(3-{[ethyl(methyl)sulfamoyl]amino}-2-fluorophenyl)-2-methyl-1,3-thiazol-5-yl]pyrimidin-2-yl}amino)pyridin-3-yl]piperazin-1-yl}methyl)piperidin-1-yl]pyridin-3-yl}piperidine-2,6-dione (43)

LCMS: C₄₂H₄₉FN₁₂O₄S₂ requires: 868.3, found: m/z=868.3 [M+H]⁺.

### Rac-N-{3-[5-(2-{[4-(1-{2-[1-(4-{[(3R)-2,6-dioxopiperidin-3-yl]amino}phenyl)piperidin-4-yl]acetyl}piperidin-4-yl)phenyl]amino}pyrimidin-4-yl)-2-methyl-1,3-thiazol-4-yl]-2-fluorophenyl}propane-1-sulfonamide (44)

LCMS: C₄₆H₅₂FN₉O₅S₂ requires: 893.4, found: m/z=894.4 [M+H]⁺.

### Rac-N-[3-(2-tert-butyl-5-{2-[(4-{4-[(1-{5-[(3R)-2,6-dioxopiperidin-3-yl]pyridin-2-yl}piperidin-4-yl)methyl]piperazin-1-yl}-3-fluorophenyl)amino]pyrimidin-4-yl}-1,3-thiazol-4-yl)-2-fluorophenyl]propane-1-sulfonamide (45)

LCMS: C₄₆H₅₄F₂N₁₀O₄S₂ requires: 912.4, found: m/z=913.6 [M+H]⁺.

### Rac-N-{3-[2-tert-butyl-5-(2-{[4-(4-{2-[1-(4-{[(3R)-2,6-dioxopiperidin-3-yl]amino}phenyl)piperidin-4-yl]acetyl}piperazin-1-yl)-3-fluorophenyl]amino}pyrimidin-4-yl)-1,3-thiazol-4-yl]-2-fluorophenyl}propane-1-sulfonamide (46)

LCMS: C₄₈H₅₆F₂N₁₀O₅S₂ requires: 954.4, found: m/z=955.2 [M+H]⁺.

### Rac-N-[3-(2-tert-butyl-5-{2-[(4-{4-[(1-{5-[(3R)-2,6-dioxopiperidin-3-yl]pyridin-2-yl}piperidin-4-yl)methyl]piperazin-1-yl}phenyl)amino]pyrimidin-4-yl}-1,3-thiazol-4-yl)-2-fluorophenyl]propane-1-sulfonamide (47)

¹H NMR (500 MHz, DMSO-d₆) δ 10.88 (s, 1H), 9.73 (s, 1H), 9.55 (s, 1H), 9.32 (s, 1H), 8.30 (d, J=5.2 Hz, 1H), 7.95 (s, 1H), 7.55 (d, J=8.3 Hz, 3H), 7.47 (t, J=6.9 Hz, 1H), 7.37 (t, J=7.9 Hz, 1H), 7.09 (s, 1H), 6.94 (d, J=8.7 Hz, 2H), 6.40 (d, J=5.1 Hz, 1H), 4.29 (d, J=13.1 Hz, 2H), 3.83 (s, 2H), 3.73 (d, J=12.7 Hz, 2H), 3.64 (d, J=11.7 Hz, 2H), 3.27-3.09 (m, 3H), 3.09-2.94 (m, 5H), 2.77-2.62 (m, 2H), 2.32-2.12 (m, 2H), 1.99 (dt, J=11.5, 5.6 Hz, 1H), 1.88 (d, J=12.5 Hz, 2H), 1.68 (p, J=7.6 Hz, 2H), 1.37-1.21 (m, 3H), 0.91 (t, J=7.4 Hz, 3H).

LCMS: C₄₆H₅₅FN₁₀O₄S₂ requires: 894.4, found: m/z=895.2 [M+H]⁺.

### Rac-N-[3-(2-tert-butyl-5-{2-[(4-{4-[2-(1-{5-[(3R)-2,6-dioxopiperidin-3-yl]pyridin-2-yl}piperidin-4-yl)acetyl]piperazin-1-yl}-3-fluorophenyl)amino]pyrimidin-4-yl}-1,3-thiazol-4-yl)-2-fluorophenyl]propane-1-sulfonamide (48)

LCMS: C₄₇H₅₄F₂N₁₀O₅S₂ requires: 940.4, found: m/z=941.3 [M+H]⁺.

### Rac-N-(3-(2-(tert-butyl)-5-(2-((4-(4-((R)-1-(5-((R)-2,6-dioxopiperidin-3-yl)pyridin-2-yl)pyrrolidine-3-carbonyl)piperazin-1-yl)-3-fluorophenyl)amino)pyrimidin-4-yl)thiazol-4-yl)-2-fluorophenyl)propane-1-sulfonamide (49)

LCMS: C₄₅H₅₀F₂N₁₀O₅S₂ requires: 912.3, found: m/z=913.6 [M+H]⁺.

### N-[3-(2-tert-butyl-5-{2-[(3-fluoro-4-{4-[(1r,4r)-4-({5-[(3RS)-2,6-dioxopiperidin-3-yl]pyridin-2-yl}oxy)cyclohexanecarbonyl]piperazin-1-yl}phenyl)amino]pyrimidin-4-yl}-1,3-thiazol-4-yl)-2-fluorophenyl]propane-1-sulfonamide (50)

LCMS: C₄₇H₅₃F₂N₉O₆S₂ requires: 941.4, found: m/z=942.1 [M+H]⁺.

### Rac-N-(3-{2-tert-butyl-5-[2-({4-[4-(2-{6-[(3R)-2,6-dioxopiperidin-3-yl]-1,2,3,4-tetrahydroisoquinolin-2-yl}acetyl)piperazin-1-yl]-3-fluorophenyl}amino)pyrimidin-4-yl]-1,3-thiazol-4-yl}-2-fluorophenyl)propane-1-sulfonamide (51)

LCMS: C₄₆H₅₁F₂N₉O₅S₂ requires: 911.3, found: m/z=912.2 [M+H]⁺.

### Rac-N-{3-[2-tert-butyl-5-(2-{[4-(4-{2-[1-(4-{[(3R)-2,6-dioxopiperidin-3-yl]amino}-2-fluorophenyl)-4-hydroxypiperidin-4-yl]acetyl}piperazin-1-yl)-3-fluorophenyl]amino}pyrimidin-4-yl)-1,3-thiazol-4-yl]-2-fluorophenyl}propane-1-sulfonamide (52)

LCMS: C₄₈H₅₅F₃N₁₀O₆S₂ requires: 988.4, found: m/z=989.2 [M+H]⁺.

### Rac-(3S)-3-[6-(3-{4-[6-({4-[2-tert-butyl-4-(3-{[ethyl(methyl)sulfamoyl]amino}-2-fluorophenyl)-1,3-thiazol-5-yl]pyrimidin-2-yl}amino)pyridin-3-yl]piperazine-1-carbonyl}azetidin-1-yl)pyridin-3-yl]piperidine-2,6-dione (53)

LCMS: C₄₃H₄₉FN₁₂O₅S₂ requires: 896.3, found: m/z=897.2 [M+H]⁺.

### Rac-N-[3-(5-{2-[(4-{1-[(1-{5-[(3R)-2,6-dioxopiperidin-3-yl]pyridin-2-yl}piperidin-4-yl)methyl]piperidin-4-yl}phenyl)amino]pyrimidin-4-yl}-2-methyl-1,3-thiazol-4-yl)-2-fluorophenyl]propane-1-sulfonamide (54)

LCMS: C₄₄H₅₀FN₉O₄S₂ requires: 851.3, found: m/z=852.2 [M+H]⁺.

### Rac-N-(3-{5-[2-({4-[1-(1-{5-[(3R)-2,6-dioxopiperidin-3-yl]pyridin-2-yl}piperidine-4-carbonyl)piperidin-4-yl]phenyl}amino)pyrimidin-4-yl]-2-methyl-1,3-thiazol-4-yl}-2-fluorophenyl)propane-1-sulfonamide (55)

LCMS: C₄₄H₄₈FN₉O₅S₂ requires: 865.3, found: m/z=866.2 [M+H]⁺.

### Rac-(3R)-3-[6-(4-{4-[6-({4-[2-tert-butyl-4-(3-{[ethyl(methyl)sulfamoyl]amino}-2-fluorophenyl)-1,3-thiazol-5-yl]pyrimidin-2-yl}amino)pyridin-3-yl]piperazine-1-carbonyl}piperidin-1-yl)pyridin-3-yl]piperidine-2,6-dione (56)

LCMS: C₄₅H₅₃FN₁₂O₅S₂ requires: 924.4, found: m/z=925.7 [M+H]⁺.

### Rac-N-(3-{2-tert-butyl-5-[2-({4-[4-(1-{5-[(3R)-2,6-dioxopiperidin-3-yl]pyridin-2-yl}piperidine-4-carbonyl)piperazin-1-yl]-3-fluorophenyl}amino)pyrimidin-4-yl]-1,3-thiazol-4-yl}-2-fluorophenyl)propane-1-sulfonamide (57)

LCMS: C₄₆H₅₂F₂N₁₀O₅S₂ requires: 926.4, found: m/z=927.2 [M+H]⁺.

### N-[3-(2-tert-butyl-5-{2-[(4-{4-[(1-{4-[(3S)-2,6-dioxopiperidin-3-yl]phenyl}piperidin-4-yl)methyl]piperazin-1-yl}phenyl)amino]pyrimidin-4-yl}-1,3-thiazol-4-yl)-2-fluorophenyl]propane-1-sulfonamide (58)

¹H NMR (500 MHz, DMSO-d₆) δ 10.78 (s, 1H), 9.46 (s, 1H), 8.28 (d, J=5.2 Hz, 1H), 7.56 (t, J=7.6 Hz, 1H), 7.47 (dd, J=23.0, 7.7 Hz, 3H), 7.36 (t, J=7.9 Hz, 1H), 7.04 (d, J=8.2 Hz, 2H), 6.88 (dd, J=18.3, 8.5 Hz, 4H), 6.35 (d, J=5.1 Hz, 1H), 3.79-3.62 (m, 3H), 3.08 (t, J=4.9 Hz, 4H), 3.01 (t, J=7.7 Hz, 2H), 2.72-2.58 (m, 4H), 2.24 (d, J=7.2 Hz, 2H), 2.20-2.08 (m, 1H), 2.02 (dt, J=13.4, 5.1 Hz, 1H), 1.82 (d, J=12.7 Hz, 2H), 1.69 (dt, J=15.2, 7.4 Hz, 3H), 1.30-1.17 (m, 2H), 0.91 (t, J=7.4 Hz, 3H).

LCMS: C₄₇H₅₆FN₉O₄S₂ requires: 893.4, found: m/z=894.4 [M+H]⁺.

### N-[3-(2-tert-butyl-5-{2-[(4-{4-[(1-{4-[(3R)-2,6-dioxopiperidin-3-yl]phenyl}piperidin-4-yl)methyl]piperazin-1-yl}phenyl)amino]pyrimidin-4-yl}-1,3-thiazol-4-yl)-2-fluorophenyl]propane-1-sulfonamide (59)

¹H NMR (500 MHz, DMSO-d₆) δ 10.80 (s, 1H), 9.73 (s, 1H), 9.56 (s, 1H), 9.34 (s, 1H), 8.30 (d, J=5.2 Hz, 1H), 7.55 (d, J=8.3 Hz, 3H), 7.47 (t, J=7.0 Hz, 1H), 7.37 (t, J=7.9 Hz, 1H), 7.17-7.05 (m, 2H), 7.05-6.84 (m, 4H), 6.40 (d, J=5.1 Hz, 1H), 3.90-3.69 (m, 6H), 3.64 (d, J=11.5 Hz, 2H), 3.18 (q, J=8.9, 6.8 Hz, 4H), 3.10-2.95 (m, 4H), 2.80 (s, 2H), 2.65 (td, J=11.9, 5.5 Hz, 1H), 2.22-1.96 (m, 3H), 1.89 (d, J=12.3 Hz, 2H), 1.67 (q, J=7.5 Hz, 2H), 1.48 (s, 9H), 1.38 (t, J=15.5 Hz, 1H), 1.27 (q, J=5.8, 4.7 Hz, 1H), 0.91 (t, J=7.4 Hz, 3H).

LCMS: C₄₇H₅₆FN₉O₄S₂ requires: 893.4, found: m/z=894.2 [M+H]⁺.

### N-[3-(5-{2-[(5-{4-[(1-{4-[(3R)-2,6-dioxopiperidin-3-yl]phenyl}piperidin-4-yl)methyl]piperazin-1-yl}pyridin-2-yl)amino]pyrimidin-4-yl}-2-methyl-1,3-thiazol-4-yl)-2-fluorophenyl]propane-1-sulfonamide (60)

LCMS: C₄₃H₄₉FN₁₀O₄S₂ requires: 852.3, found: m/z=853.2 [M+H]⁺.

### N-[3-(5-{2-[(5-{4-[(1-{4-[(3S)-2,6-dioxopiperidin-3-yl]phenyl}piperidin-4-yl)methyl]piperazin-1-yl}pyridin-2-yl)amino]pyrimidin-4-yl}-2-methyl-1,3-thiazol-4-yl)-2-fluorophenyl]propane-1-sulfonamide (61)

LCMS: C₄₃H₄₉FN₁₀O₄S₂ requires: 852.3, found: m/z=853.2 [M+H]⁺.

### Rac-N-[3-(5-{2-[(5-{4-[(1-{5-[(3R)-2,6-dioxopiperidin-3-yl]pyridin-2-yl}piperidin-4-yl)methyl]piperazin-1-yl}pyridin-2-yl)amino]pyrimidin-4-yl}-2-methyl-1,3-thiazol-4-yl)-2-fluorophenyl]propane-1-sulfonamide (62)

LCMS: C₄₂H₄₈FN₁₁O₄S₂ requires: 853.3, found: m/z=854.2 [M+H]⁺.

### N-[3-(5-{2-[(4-{1-[(1-{4-[(3R)-2,6-dioxopiperidin-3-yl]phenyl}piperidin-4-yl)methyl]piperidin-4-yl}phenyl)amino]pyrimidin-4-yl}-2-methyl-1,3-thiazol-4-yl)-2-fluorophenyl]propane-1-sulfonamide (63)

LCMS: C₄₅H₅₁FN₈O₄S₂ requires: 850.3, found: m/z=851.2 [M+H]⁺.

### N-[3-(5-{2-[(4-{1-[(1-{4-[(3S)-2,6-dioxopiperidin-3-yl]phenyl}piperidin-4-yl)methyl]piperidin-4-yl}phenyl)amino]pyrimidin-4-yl}-2-methyl-1,3-thiazol-4-yl)-2-fluorophenyl]propane-1-sulfonamide (64)

LCMS: C₄₅H₅₁FN₈O₄S₂ requires: 850.3, found: m/z=851.2 [M+H]⁺.

### Rac-N-(3-{2-tert-butyl-5-[2-({4-[4-(1-{4-[(3R)-2,6-dioxopiperidin-3-yl]phenyl}piperidine-4-carbonyl)piperazin-1-yl]-3-fluorophenyl}amino)pyrimidin-4-yl]-1,3-thiazol-4-yl}-2-fluorophenyl)propane-1-sulfonamide (65)

LCMS: C₄₇H₅₃F₂N₉O₅S₂ requires: 925.4, found: m/z=926.2 [M+H]⁺.

### 5-(4-{[4-({4-[(4-{2-tert-butyl-4-[2-fluoro-3-(propane-1-sulfonamido)phenyl]-1,3-thiazol-5-yl}pyrimidin-2-yl)amino]-1H-pyrazol-1-yl}methyl)piperidin-1-yl]methyl}piperidin-1-yl)-N-[(3R)-2,6-dioxopiperidin-3-yl]pyridine-2-carboxamide (66)

LCMS: C₄₆H₅₇FN₁₂O₅S₂ requires: 940.4, found: m/z=941.2 [M+H]⁺.

### 5-{4-[(4-{4-[(4-{2-tert-butyl-4-[2-fluoro-3-(propane-1-sulfonamido)phenyl]-1,3-thiazol-5-yl}pyrimidin-2-yl)amino]-1H-pyrazol-1-yl}piperidin-1-yl)methyl]piperidin-1-yl}-N-[(3R)-2,6-dioxopiperidin-3-yl]pyridine-2-carboxamide (67)

LCMS: C₄₅H₅₅FN₁₂O₅S₂ requires: 926.4, found: m/z=927.3 [M+H]⁺.

### Rac-N-{3-[2-tert-butyl-5-(2-{[3-({1-[(1-{5-[(3R)-2,6-dioxopiperidin-3-yl]pyridin-2-yl}-4-methylpiperidin-4-yl)methyl]piperidin-4-yl}oxy)phenyl]amino}pyrimidin-4-yl)-1,3-thiazol-4-yl]-2-fluorophenyl}propane-1-sulfonamide (68)

LCMS: C₄₈H₅₈FN₉O₅S₂ requires: 923.4, found: m/z=924.3 [M+H]⁺.

### N-(3-{2-tert-butyl-5-[2-({3-[(1-{2-[(3S)-1-{5-[(3RS)-2,6-dioxopiperidin-3-yl]pyridin-2-yl}pyrrolidin-3-yl]ethyl}piperidin-4-yl)oxy]phenyl}amino)pyrimidin-4-yl]-1,3-thiazol-4-yl}-2-fluorophenyl)propane-1-sulfonamide (69)

LCMS: C₄₇H₅₆FN₉O₅S₂ requires: 909.4, found: m/z=910.6 [M+H]⁺.

### N-(3-{2-tert-butyl-5-[2-({3-[(1-{2-[(3R)-1-{5-[(3RS)-2,6-dioxopiperidin-3-yl]pyridin-2-yl}pyrrolidin-3-yl]ethyl}piperidin-4-yl)oxy]phenyl}amino)pyrimidin-4-yl]-1,3-thiazol-4-yl}-2-fluorophenyl)propane-1-sulfonamide (70)

LCMS: C₄₇H₅₆FN₉O₅S₂ requires: 909.4, found: m/z=910.3 [M+H]⁺.

### Rac-N-{3-[2-tert-butyl-5-(2-{[3-({1-[(1-{2-[(3R)-2,6-dioxopiperidin-3-yl]-1-oxo-1,2-dihydroisoquinolin-6-yl}piperidin-4-yl)methyl]piperidin-4-yl}oxy)phenyl]amino}pyrimidin-4-yl)-1,3-thiazol-4-yl]-2-fluorophenyl}propane-1-sulfonamide (71)

LCMS: C₅₁H₅₈FN₉O₆S₂ requires: 975.4, found: m/z=976.5 [M+H]⁺.

### N-(3-(2-(tert-butyl)-5-(2-((3-((1-(((1R,4r)-4-(4-((RS)-2,6-dioxopiperidin-3-yl)phenoxy)cyclohexyl)methyl)piperidin-4-yl)oxy)phenyl)amino)pyrimidin-4-yl)thiazol-4-yl)-2-fluorophenyl)propane-1-sulfonamide (72)

LCMS: C₄₉H₅₈FN₇O₆S₂ requires: 923.4, found: m/z=924.2 [M+H]⁺.

### Rac-N-[3-(2-tert-butyl-5-{2-[(3-{1-[(1-{5-[(3R)-2,6-dioxopiperidin-3-yl]pyridin-2-yl}-4-methylpiperidin-4-yl)methyl]piperidin-4-yl}phenyl)amino]pyrimidin-4-yl}-1,3-thiazol-4-yl)-2-fluorophenyl]propane-1-sulfonamide (73)

LCMS: C₄₈H₅₈FN₉O₄S₂ requires: 907.4, found: m/z=908.9 [M+H]⁺.

### N-{3-[2-tert-butyl-5-(2-{[3-(1-{2-[(3S)-1-{5-[(3RS)-2,6-dioxopiperidin-3-yl]pyridin-2-yl}pyrrolidin-3-yl]ethyl}piperidin-4-yl)phenyl]amino}pyrimidin-4-yl)-1,3-thiazol-4-yl]-2-fluorophenyl}propane-1-sulfonamide (74)

LCMS: C₄₇H₅₆FN₉O₄S₂ requires: 893.4, found: m/z=894.2 [M+H]⁺.

### N-{3-[2-tert-butyl-5-(2-{[3-(1-{2-[(3R)-1-{5-[(3RS)-2,6-dioxopiperidin-3-yl]pyridin-2-yl}pyrrolidin-3-yl]ethyl}piperidin-4-yl)phenyl]amino}pyrimidin-4-yl)-1,3-thiazol-4-yl]-2-fluorophenyl}propane-1-sulfonamide (75)

LCMS: C₄₇H₅₆FN₉O₄S₂ requires: 893.4, found: m/z=894.2 [M+H]⁺.

### Rac-N-[3-(2-tert-butyl-5-{2-[(3-{1-[(1-{2-[(3R)-2,6-dioxopiperidin-3-yl]-1-oxo-1,2-dihydroisoquinolin-6-yl}piperidin-4-yl)methyl]piperidin-4-yl}phenyl)amino]pyrimidin-4-yl}-1,3-thiazol-4-yl)-2-fluorophenyl]propane-1-sulfonamide (76)

LCMS: C₅₁H₅₈FN₉O₄S₂ requires: 959.4, found: m/z=960.3 [M+H]⁺.

### N-{3-[2-tert-butyl-5-(2-{[1-(1-{2-[(3S)-1-{5-[(3RS)-2,6-dioxopiperidin-3-yl]pyridin-2-yl}pyrrolidin-3-yl]ethyl}piperidin-4-yl)-1H-pyrazol-4-yl]amino}pyrimidin-4-yl)-1,3-thiazol-4-yl]-2-fluorophenyl}propane-1-sulfonamide (77)

LCMS: C₄₄H₅₄FN₁₁O₄S₂ requires: 883.4, found: m/z=884.3 [M+H]⁺.

### N-{3-[2-tert-butyl-5-(2-{[1-(1-{2-[(3R)-1-{5-[(3RS)-2,6-dioxopiperidin-3-yl]pyridin-2-yl}pyrrolidin-3-yl]ethyl}piperidin-4-yl)-1H-pyrazol-4-yl]amino}pyrimidin-4-yl)-1,3-thiazol-4-yl]-2-fluorophenyl}propane-1-sulfonamide (78)

LCMS: C₄₄H₅₄FN₁₁O₄S₂ requires: 883.4, found: m/z=884.6 [M+H]⁺.

### Rac-5-{4-[(4-{4-[(4-{2-tert-butyl-4-[2-fluoro-3-(propane-1-sulfonamido)phenyl]-1,3-thiazol-5-yl}pyrimidin-2-yl)amino]-1H-pyrazol-1-yl}piperidin-1-yl)methyl]piperidin-1-yl}-N-[(3R)-2,6-dioxopiperidin-3-yl]pyridine-2-carboxamide (79)

LCMS: C₄₅H₅₅FN₁₂O₅S₂ requires: 926.4, found: m/z=927.2 [M+H]⁺.

### Rac-N-[3-(2-tert-butyl-5-{2-[(1-f{1-[(1-{2-[(3R)-2,6-dioxopiperidin-3-yl]-1-oxo-1,2-dihydroisoquinolin-6-yl}piperidin-4-yl)methyl]piperidin-4-yl}-1H-pyrazol-4-yl)amino]pyrimidin-4-yl}-1,3-thiazol-4-yl)-2-fluorophenyl]propane-1-sulfonamide (80)

LCMS: C₄₈H₅₆FN₁₁O₅S₂ requires: 949.4, found: m/z=950.3 [M+H]⁺.

### N-(3-{2-tert-butyl-5-[2-({1-[(1-{2-[(3R)-1-{5-[(3RS)-2,6-dioxopiperidin-3-yl]pyridin-2-yl}pyrrolidin-3-yl]ethyl}piperidin-4-yl)methyl]-1H-pyrazol-4-yl}amino)pyrimidin-4-yl]-1,3-thiazol-4-yl}-2-fluorophenyl)propane-1-sulfonamide (81)

LCMS: C₄₅H₅₆FN₁₁O₄S₂ requires: 897.4, found: m/z=898.2 [M+H]⁺.

### Rac-5-(4-{[4-({4-[(4-{2-tert-butyl-4-[2-fluoro-3-(propane-1-sulfonamido)phenyl]-1,3-thiazol-5-yl}pyrimidin-2-yl)amino]-1H-pyrazol-1-yl}methyl)piperidin-1-yl]methyl}piperidin-1-yl)-N-[(3R)-2,6-dioxopiperidin-3-yl]pyridine-2-carboxamide (82)

LCMS: C₄₆H₅₇FN₁₂O₅S₂ requires: 940.4, found: m/z=941.3 [M+H]⁺.

### Rac-N-{3-[2-tert-butyl-5-(2-{[1-({1-[(1-{2-[(3R)-2,6-dioxopiperidin-3-yl]-1-oxo-1,2-dihydroisoquinolin-6-yl}piperidin-4-yl)methyl]piperidin-4-yl}methyl)-1H-pyrazol-4-yl]amino}pyrimidin-4-yl)-1,3-thiazol-4-yl]-2-fluorophenyl}propane-1-sulfonamide (83)

LCMS: C₄₉H₅₈FN₁₁O₅S₂ requires: 963.4, found: m/z=964.2 [M+H]⁺.

### N-(3-{2-tert-butyl-5-[2-({1-[(1-{[(1r,4r)-4-{4-[(3RS)-2,6-dioxopiperidin-3-yl]phenoxy}cyclohexyl]methyl}piperidin-4-yl)methyl]-1H-pyrazol-4-yl}amino)pyrimidin-4-yl]-1,3-thiazol-4-yl}-2-fluorophenyl)propane-1-sulfonamide (84)

LCMS: C₄₇H₅₈FN₉O₅S₂ requires: 911.4, found: m/z=912.2 [M+H]⁺.

### Rac-N-(3-{2-tert-butyl-5-[2-({2-[(1-{2-[(3R)-2,6-dioxopiperidin-3-yl]-1-oxo-1,2-dihydroisoquinolin-6-yl}piperidin-4-yl)methyl]-1,2,3,4-tetrahydroisoquinolin-6-yl}amino)pyrimidin-4-yl]-1,3-thiazol-4-yl}-2-fluorophenyl)propane-1-sulfonamide (85)

LCMS: C₄₉H₅₄FN₉O₅S₂ requires: 931.4, found: m/z=932.3 [M+H]⁺.

### (2S,4R)-1-[(2S)-2-[5-(4-{4-[(4-{2-tert-butyl-4-[2-fluoro-3-(propane-1-sulfonamido)phenyl]-1,3-thiazol-5-yl}pyrimidin-2-yl)amino]phenyl}piperidin-1-yl)-5-oxopentanamido]-3,3-dimethylbutanoyl]-4-hydroxy-N-[(1S)-1-[4-(4-methyl-1,3-thiazol-5-yl)phenyl]ethyl]pyrrolidine-2-carboxamide (86)

LCMS: C₅₉H₇₃FN₁₀O₇S₃ requires: 1148.5, found: m/z=1149.3 [M+H]⁺.

### N-[3-(2-tert-butyl-5-{2-[(4-{4-[(1-{4-[(3R)-2,6-dioxopiperidin-3-yl]phenyl}piperidin-4-yl)methyl]piperazin-1-yl}-3-fluorophenyl)amino]pyrimidin-4-yl}-1,3-thiazol-4-yl)-2-fluorophenyl]propane-1-sulfonamide (87)

LCMS: C₄₇H₅₅F₂N₉O₄S₂ requires: 911.4, found: m/z=912.6 [M+H]⁺.

### N-[3-(2-tert-butyl-5-{2-[(4-{4-[(1-{4-[(3S)-2,6-dioxopiperidin-3-yl]phenyl}piperidin-4-yl)methyl]piperazin-1-yl}-3-fluorophenyl)amino]pyrimidin-4-yl}-1,3-thiazol-4-yl)-2-fluorophenyl]propane-1-sulfonamide (88)

LCMS: C₄₇H₅₅F₂N₉O₄S₂ requires: 911.4, found: m/z=912.3 [M+H]⁺.

### N-[3-(2-tert-butyl-5-{2-[(4-{1-[(1-{4-[(3R)-2,6-dioxopiperidin-3-yl]phenyl}piperidin-4-yl)methyl]piperidin-4-yl}phenyl)amino]pyrimidin-4-yl}-1,3-thiazol-4-yl)-2-fluorophenyl]propane-1-sulfonamide (89)

LCMS: C₄₈H₅₇FN₈O₄S₂ requires: 892.4, found: m/z=893.2 [M+H]⁺.

### N-[3-(2-tert-butyl-5-{2-[(4-{1-[(1-{4-[(3S)-2,6-dioxopiperidin-3-yl]phenyl}piperidin-4-yl)methyl]piperidin-4-yl}phenyl)amino]pyrimidin-4-yl}-1,3-thiazol-4-yl)-2-fluorophenyl]propane-1-sulfonamide (90)

LCMS: C₄₈H₅₇FN₈O₄S₂ requires: 892.4, found: m/z=893.2 [M+H]⁺.

### N-(3-{2-tert-butyl-5-[2-({3-[(1-{2-[(3R)-1-{4-[(3RS)-2,6-dioxopiperidin-3-yl]phenyl}pyrrolidin-3-yl]acetyl}piperidin-4-yl)oxy]phenyl}amino)pyrimidin-4-yl]-1,3-thiazol-4-yl}-2-fluorophenyl)propane-1-sulfonamide (91)

LCMS: C₄₈H₅₅FN₈O₆S₂ requires: 922.4, found: m/z=923.3 [M+H]⁺.

### N-(3-{2-tert-butyl-5-[2-({3-[(1-{2-[(3S)-1-{4-[(3RS)-2,6-dioxopiperidin-3-yl]phenyl}pyrrolidin-3-yl]acetyl}piperidin-4-yl)oxy]phenyl}amino)pyrimidin-4-yl]-1,3-thiazol-4-yl}-2-fluorophenyl)propane-1-sulfonamide (92)

LCMS: C₄₈H₅₅FN₈O₆S₂ requires: 922.4, found: m/z=923.2 [M+H]⁺.

### Rac-N-{3-[2-tert-butyl-5-(2-{[3-({1-[2-(4-{4-[(3R)-2,6-dioxopiperidin-3-yl]pyridin-2-yl}piperazin-1-yl)acetyl]piperidin-4-yl}oxy)phenyl]amino}pyrimidin-4-yl)-1,3-thiazol-4-yl]-2-fluorophenyl}propane-1-sulfonamide (93)

LCMS: C₄₇H₅₅FN₁₀O₆S₂ requires: 938.4, found: m/z=939.5 [M+H]⁺.

### N-{3-[2-tert-butyl-5-(2-{[3-({1-[(3R)-1-{5-[(3RS)-2,6-dioxopiperidin-3-yl]pyridin-2-yl}pyrrolidine-3-carbonyl]piperidin-4-yl}oxy)phenyl]amino}pyrimidin-4-yl)-1,3-thiazol-4-yl]-2-fluorophenyl}propane-1-sulfonamide (94)

LCMS: C₄₆H₅₂FN₉O₆S₂ requires: 909.3, found: m/z=910.2 [M+H]⁺.

### Rac-5-[4-(4-{3-[(4-{2-tert-butyl-4-[2-fluoro-3-(propane-1-sulfonamido)phenyl]-1,3-thiazol-5-yl}pyrimidin-2-yl)amino]phenoxy}piperidine-1-carbonyl)piperidin-1-yl]-N-[(3R)-2,6-dioxopiperidin-3-yl]pyridine-2-carboxamide (95)

LCMS: C₄₈H₅₅FN₁₀O₇S₂ requires: 966.4, found: m/z=967.2 [M+H]⁺.

### Rac-N-(3-{2-tert-butyl-5-[2-({3-[(1-{2-[4-(4-{[(3R)-2,6-dioxopiperidin-3-yl]amino}phenyl)piperidin-1-yl]acetyl}piperidin-4-yl)oxy]phenyl}amino)pyrimidin-4-yl]-1,3-thiazol-4-yl}-2-fluorophenyl)propane-1-sulfonamide (96)

LCMS: C₄₉H₅₈FN₉O₆S₂ requires: 951.4, found: m/z=952.2 [M+H]⁺.

### N-{3-[2-tert-butyl-5-(2-{[3-({1-[(1r,4r)-4-({5-[(3RS)-2,6-dioxopiperidin-3-yl]pyridin-2-yl}oxy)cyclohexanecarbonyl]piperidin-4-yl}oxy)phenyl]amino}pyrimidin-4-yl)-1,3-thiazol-4-yl]-2-fluorophenyl}propane-1-sulfonamide (97)

LCMS: C₄₈H₅₅FN₈O₇S₂ requires: 938.4, found: m/z=939.2 [M+H]⁺.

### N-{3-[2-tert-butyl-5-(2-{[3-({1-[(1r,4r)-4-{4-[(3RS)-2,6-dioxopiperidin-3-yl]phenoxy}cyclohexanecarbonyl]piperidin-4-yl}oxy)phenyl]amino}pyrimidin-4-yl)-1,3-thiazol-4-yl]-2-fluorophenyl}propane-1-sulfonamide (98)

LCMS: C₄₉H₅₆FN₇O₇S₂ requires: 937.4, found: m/z=955.6 [M+NH₄]⁺.

### Rac-N-[3-(2-tert-butyl-5-{2-[(3-{[1-(1-{3-[(3R)-2,6-dioxopiperidin-3-yl]phenyl}piperidine-4-carbonyl)piperidin-4-yl]oxy}phenyl)amino]pyrimidin-4-yl}-1,3-thiazol-4-yl)-2-fluorophenyl]propane-1-sulfonamide (99)

LCMS: C₄₈H₅₅FN₈O₆S₂ requires: 922.4, found: m/z=923.7 [M+H]⁺.

### Rac-N-[3-(2-tert-butyl-5-{2-[(3-{[1-(1-{4-[(3R)-2,6-dioxopiperidin-3-yl]pyridin-2-yl}piperidine-4-carbonyl)piperidin-4-yl]oxy}phenyl)amino]pyrimidin-4-yl}-1,3-thiazol-4-yl)-2-fluorophenyl]propane-1-sulfonamide (100)

LCMS: C₄₇H₅₄FN₉O₆S₂ requires: 923.4, found: m/z=924.3 [M+H]⁺.

### Rac-N-[3-(2-tert-butyl-5-{2-[(3-{[1-(2-{6-[(3R)-2,6-dioxopiperidin-3-yl]-1,2,3,4-tetrahydroisoquinolin-2-yl}acetyl)piperidin-4-yl]oxy}phenyl)amino]pyrimidin-4-yl}-1,3-thiazol-4-yl)-2-fluorophenyl]propane-1-sulfonamide (101)

LCMS: C₄₇H₅₃FN₈O₆S₂ requires: 908.4, found: m/z=909.0 [M+H]⁺.

### N-[3-(2-tert-butyl-5-{2-[(3-{1-[(3R)-1-{5-[(3RS)-2,6-dioxopiperidin-3-yl]pyridin-2-yl}pyrrolidine-3-carbonyl]piperidin-4-yl}phenyl)amino]pyrimidin-4-yl}-1,3-thiazol-4-yl)-2-fluorophenyl]propane-1-sulfonamide (102)

LCMS: C₄₆H₅₂FN₉O₅S₂ requires: 893.4, found: m/z=894.2 [M+H]⁺.

### Rac-N-{3-[2-tert-butyl-5-(2-{[3-(1-{2-[4-(4-{[(3R)-2,6-dioxopiperidin-3-yl]amino}phenyl)piperidin-1-yl]acetyl}piperidin-4-yl)phenyl]amino}pyrimidin-4-yl)-1,3-thiazol-4-yl]-2-fluorophenyl}propane-1-sulfonamide (103)

LCMS: C₄₉H₅₈FN₉O₅S₂ requires: 935.4, found: m/z=936.2 [M+H]⁺.

### Rac-N-(3-{2-tert-butyl-5-[2-({3-[1-(2-{6-[(3R)-2,6-dioxopiperidin-3-yl]-1,2,3,4-tetrahydroisoquinolin-2-yl}acetyl)piperidin-4-yl]phenyl}amino)pyrimidin-4-yl]-1,3-thiazol-4-yl}-2-fluorophenyl)propane-1-sulfonamide (104)

LCMS: C₄₇H₅₃FN₈O₅S₂ requires: 892.4, found: m/z=893.2 [M+H]⁺.

### N-{3-[2-tert-butyl-5-(2-{[1-(1-{2-[(3S)-1-{4-[(3RS)-2,6-dioxopiperidin-3-yl]phenyl}pyrrolidin-3-yl]acetyl}piperidin-4-yl)-1H-pyrazol-4-yl]amino}pyrimidin-4-yl)-1,3-thiazol-4-yl]-2-fluorophenyl}propane-1-sulfonamide (105)

LCMS: C₄₅H₅₃FN₁₀O₅S₂ requires: 896.4, found: m/z=897.0 [M+H]⁺.

### N-[3-(2-tert-butyl-5-{2-[(1-{1-[(3R)-1-{5-[(3RS)-2,6-dioxopiperidin-3-yl]pyridin-2-yl}pyrrolidine-3-carbonyl]piperidin-4-yl}-1H-pyrazol-4-yl)amino]pyrimidin-4-yl}-1,3-thiazol-4-yl)-2-fluorophenyl]propane-1-sulfonamide (106)

LCMS: C₄₃H₅₀FN₁₁O₅S₂ requires: 883.3, found: m/z=884.3 [M+H]⁺.

### Rac-5-[4-(4-{4-[(4-{2-tert-butyl-4-[2-fluoro-3-(propane-1-sulfonamido)phenyl]-1,3-thiazol-5-yl}pyrimidin-2-yl)amino]-1H-pyrazol-1-yl}piperidine-1-carbonyl)piperidin-1-yl]-N-[(3R)-2,6-dioxopiperidin-3-yl]pyridine-2-carboxamide (107)

LCMS: C₄₅H₅₃FN₁₂O₆S₂ requires: 940.4, found: m/z=941.3 [M+H]⁺.

### Rac-N-{3-[2-tert-butyl-5-(2-{[1-(1-{2-[4-(4-{[(3R)-2,6-dioxopiperidin-3-yl]amino}phenyl)piperidin-1-yl]acetyl}piperidin-4-yl)-1H-pyrazol-4-yl]amino}pyrimidin-4-yl)-1,3-thiazol-4-yl]-2-fluorophenyl}propane-1-sulfonamide (108)

LCMS: C₄₆H₅₆FN₁₁O₅S₂ requires: 925.4, found: m/z=926.1 [M+H]⁺.

### N-[3-(2-tert-butyl-5-{2-[(1-{1-[(1r,4r)-4-({5-[(3RS)-2,6-dioxopiperidin-3-yl]pyridin-2-yl}oxy)cyclohexanecarbonyl]piperidin-4-yl}-1H-pyrazol-4-yl)amino]pyrimidin-4-yl}-1,3-thiazol-4-yl)-2-fluorophenyl]propane-1-sulfonamide (109)

LCMS: C₄₅H₅₃FN₁₁O₆S₂ requires: 912.4, found: m/z=913.2 [M+H]⁺.

### N-[3-(2-tert-butyl-5-{2-[(1-{1-[(1r,4r)-4-{4-[(3RS)-2,6-dioxopiperidin-3-yl]phenoxy}cyclohexanecarbonyl]piperidin-4-yl}-1H-pyrazol-4-yl)amino]pyrimidin-4-yl}-1,3-thiazol-4-yl)-2-fluorophenyl]propane-1-sulfonamide (110)

LCMS: C₄₆H₅₄FN₉O₆S₂ requires: 911.4, found: m/z=912.2 [M+H]⁺.

### N-(3-{2-tert-butyl-5-[2-({1-[(1-{2-[(3R)-1-{4-[(3RS)-2,6-dioxopiperidin-3-yl]phenyl}pyrrolidin-3-yl]acetyl}piperidin-4-yl)methyl]-1H-pyrazol-4-yl}amino)pyrimidin-4-yl]-1,3-thiazol-4-yl}-2-fluorophenyl)propane-1-sulfonamide (111)

LCMS: C₄₆H₅₅FN₁₀O₅S₂ requires: 910.4, found: m/z=911.4 [M+H]⁺.

### N-(3-{2-tert-butyl-5-[2-({1-[(1-{2-[(3S)-1-{4-[(3RS)-2,6-dioxopiperidin-3-yl]phenyl}pyrrolidin-3-yl]acetyl}piperidin-4-yl)methyl]-1H-pyrazol-4-yl}amino)pyrimidin-4-yl]-1,3-thiazol-4-yl}-2-fluorophenyl)propane-1-sulfonamide (112)

LCMS: C₄₆H₅₅FN₁₀O₅S₂ requires: 910.4, found: m/z=911.4 [M+H]⁺.

### Rac-N-{3-[2-tert-butyl-5-(2-{[1-({1-[2-(4-{4-[(3R)-2,6-dioxopiperidin-3-yl]pyridin-2-yl}piperazin-1-yl)acetyl]piperidin-4-yl}methyl)-1H-pyrazol-4-yl]amino}pyrimidin-4-yl)-1,3-thiazol-4-yl]-2-fluorophenyl}propane-1-sulfonamide (113)

LCMS: C₄₅H₅₅FN₁₂O₅S₂ requires: 926.4, found: m/z=927.2 [M+H]⁺.

### N-{3-[2-tert-butyl-5-(2-{[1-({1-[(3R)-1-{5-[(3RS)-2,6-dioxopiperidin-3-yl]pyridin-2-yl}pyrrolidine-3-carbonyl]piperidin-4-yl}methyl)-1H-pyrazol-4-yl]amino}pyrimidin-4-yl)-1,3-thiazol-4-yl]-2-fluorophenyl}propane-1-sulfonamide (114)

LCMS: C₄₄H₅₂FN₁₁O₅S₂ requires: 897.4, found: m/z=898.4 [M+H]⁺.

### Rac-5-{4-[4-({4-[(4-{2-tert-butyl-4-[2-fluoro-3-(propane-1-sulfonamido)phenyl]-1,3-thiazol-5-yl}pyrimidin-2-yl)amino]-1H-pyrazol-1-yl}methyl)piperidine-1-carbonyl]piperidin-1-yl}-N-[(3R)-2,6-dioxopiperidin-3-yl]pyridine-2-carboxamide (115)

LCMS: C₄₆H₅₅FN₁₂O₆S₂ requires: 954.4, found: m/z=955.3 [M+H]⁺.

### Rac-N-(3-{2-tert-butyl-5-[2-({1-[(1-{2-[4-(4-{[(3R)-2,6-dioxopiperidin-3-yl]amino}phenyl)piperidin-1-yl]acetyl}piperidin-4-yl)methyl]-1H-pyrazol-4-yl}amino)pyrimidin-4-yl]-1,3-thiazol-4-yl}-2-fluorophenyl)propane-1-sulfonamide (116)

LCMS: C₄₇H₅₈FN₁₁O₅S₂ requires: 939.4, found: m/z=940.5 [M+H]⁺.

### N-{3-[2-tert-butyl-5-(2-{[1-({1-[(1r,4r)-4-({5-[(3RS)-2,6-dioxopiperidin-3-yl]pyridin-2-yl}oxy)cyclohexanecarbonyl]piperidin-4-yl}methyl)-1H-pyrazol-4-yl]amino}pyrimidin-4-yl)-1,3-thiazol-4-yl]-2-fluorophenyl}propane-1-sulfonamide (117)

LCMS: C₄₆H₅₅FN₁₀O₆S₂ requires: 926.4, found: m/z=927.2 [M+H]⁺.

### N-{3-[2-tert-butyl-5-(2-{[1-({1-[(1r,4r)-4-{4-[(3RS)-2,6-dioxopiperidin-3-yl]phenoxy}cyclohexanecarbonyl]piperidin-4-yl}methyl)-1H-pyrazol-4-yl]amino}pyrimidin-4-yl)-1,3-thiazol-4-yl]-2-fluorophenyl}propane-1-sulfonamide (118)

LCMS: C₄₇H₅₆FN₉O₆S₂ requires: 925.4, found: m/z=926.2 [M+H]⁺.

### Rac-N-[3-(2-tert-butyl-5-{2-[(1-{[1-(2-{6-[(3R)-2,6-dioxopiperidin-3-yl]-1,2,3,4-tetrahydroisoquinolin-2-yl}acetyl)piperidin-4-yl]methyl}-1H-pyrazol-4-yl)amino]pyrimidin-4-yl}-1,3-thiazol-4-yl)-2-fluorophenyl]propane-1-sulfonamide (119)

LCMS: C₄₅H₅₃FN₁₀O₅S₂ requires: 896.4, found: m/z=897.5 [M+H]⁺.

### Rac-N-(3-{2-tert-butyl-5-[2-({2-[2-(4-{4-[(3R)-2,6-dioxopiperidin-3-yl]pyridin-2-yl}piperazin-1-yl)acetyl]-1,2,3,4-tetrahydroisoquinolin-6-yl}amino)pyrimidin-4-yl]-1,3-thiazol-4-yl}-2-fluorophenyl)propane-1-sulfonamide (120)

LCMS: C₄₅H₅₁FN₁₀O₅S₂ requires: 894.3, found: m/z=895.2 [M+H]⁺.

### N-(3-{2-tert-butyl-5-[2-({2-[(3R)-1-{5-[(3RS)-2,6-dioxopiperidin-3-yl]pyridin-2-yl}pyrrolidine-3-carbonyl]-1,2,3,4-tetrahydroisoquinolin-6-yl}amino)pyrimidin-4-yl]-1,3-thiazol-4-yl}-2-fluorophenyl)propane-1-sulfonamide (121)

LCMS: C₄₄H₄₈FN₉O₅S₂ requires: 865.3, found: m/z=866.3 [M+H]⁺.

### Rac-5-(4-{6-[(4-{2-tert-butyl-4-[2-fluoro-3-(propane-1-sulfonamido)phenyl]-1,3-thiazol-5-yl}pyrimidin-2-yl)amino]-1,2,3,4-tetrahydroisoquinoline-2-carbonyl}piperidin-1-yl)-N-[(3R)-2,6-dioxopiperidin-3-yl]pyridine-2-carboxamide (122)

LCMS: C₄₆H₅₁FN₁₀O₆S₂ requires: 922.3, found: m/z=923.1 [M+H]⁺.

### Rac-N-[3-(2-tert-butyl-5-{2-[(2-{2-[4-(4-{[(3R)-2,6-dioxopiperidin-3-yl]amino}phenyl)piperidin-1-yl]acetyl}-1,2,3,4-tetrahydroisoquinolin-6-yl)amino]pyrimidin-4-yl}-1,3-thiazol-4-yl)-2-fluorophenyl]propane-1-sulfonamide (123)

LCMS: C₄₇H₅₄FN₉O₅S₂ requires: 907.4, found: m/z=908.2 [M+H]⁺.

### Rac-(3S)-3-(2-{[(1r,4r)-4-{4-[6-({4-[2-tert-butyl-4-(3-{[ethyl(methyl)sulfamoyl]amino}-2-fluorophenyl)-1,3-thiazol-5-yl]pyrimidin-2-yl}amino)pyridin-3-yl]piperazine-1-carbonyl}cyclohexyl]methyl}-1,2,3,4-tetrahydroisoquinolin-7-yl)piperidine-2,6-dione (124)

LCMS: C₅₁H₆₂FN₁₁O₅S₂ requires: 991.4, found: m/z=992.3 [M+H]⁺.

### Rac-(3S)-3-(2-{[(1r,4r)-4-{4-[6-({4-[2-tert-butyl-4-(3-{[ethyl(methyl)sulfamoyl]amino}-2-fluorophenyl)-1,3-thiazol-5-yl]pyrimidin-2-yl}amino)pyridin-3-yl]piperazine-1-carbonyl}cyclohexyl]methyl}-1,2,3,4-tetrahydroisoquinolin-6-yl)piperidine-2,6-dione (125)

LCMS: C₅₁H₆₂FN₁₁O₅S₂ requires: 991.4, found: m/z=992.2 [M+H]⁺.

### Rac-(3S)-3-{6-[4-({4-[6-({4-[2-tert-butyl-4-(3-{[ethyl(methyl)sulfamoyl]amino}-2-fluorophenyl)-1,3-thiazol-5-yl]pyrimidin-2-yl}amino)pyridin-3-yl]piperazin-1-yl}methyl)piperidin-1-yl]pyridin-3-yl}piperidine-2,6-dione (126)

¹H NMR (500 MHz, CD₃CN) δ 10.30 (s, 1H), 8.82 (s, 1H), 8.50 (d, J=5.4 Hz, 1H), 7.95 (d, J=2.3 Hz, 1H), 7.91 (dd, J=9.7, 2.9 Hz, 1H), 7.80 (dd, J=9.6, 2.3 Hz, 1H), 7.69 (d, J=9.6 Hz, 1H), 7.63-7.55 (m, 2H), 7.49 (dd, J=14.5, 7.6 Hz, 2H), 7.37 (t, J=7.9 Hz, 1H), 7.19 (d, J=9.5 Hz, 1H), 4.22 (d, J=13.7 Hz, 2H), 3.83 (dd, J=12.7, 5.1 Hz, 1H), 3.24 (t, J=12.8 Hz, 2H), 3.17 (q, J=7.1 Hz, 2H), 3.08 (d, J=6.8 Hz, 2H), 2.76 (s, 3H), 2.74-2.69 (m, 2H), 2.40-2.09 (m, 2H), 2.09-2.02 (m, 2H), 1.54 (s, 9H), 1.50-1.40 (m, 2H), 1.09 (t, J=7.1 Hz, 3H).

LCMS: C₄₅H₅₅FN₁₂O₄S₂ requires: 910.4, found: m/z=911.2 [M+H]⁺.

### (3S)-3-{4-[4-({4-[6-({4-[2-tert-butyl-4-(3-{[ethyl(methyl)sulfamoyl]amino}-2-fluorophenyl)-1,3-thiazol-5-yl]pyrimidin-2-yl}amino)pyridin-3-yl]piperazin-1-yl}methyl)piperidin-1-yl]phenyl}piperidine-2,6-dione (127)

¹H NMR (500 MHz, DMSO-d₆) δ 10.79 (s, 1H), 9.69 (s, 1H), 9.32 (s, 1H), 8.41 (s, 1H), 8.03 (s, 1H), 7.84 (s, 1H), 7.55 (t, J=8.0 Hz, 1H), 7.48-7.26 (m, 2H), 7.09 (d, J=7.7 Hz, 2H), 6.97 (s, 2H), 6.54 (s, 1H), 3.81 (d, J=12.6 Hz, 2H), 3.77-3.62 (m, 2H), 3.29-2.99 (m, 6H), 2.73 (d, J=28.4 Hz, 2H), 2.66 (d, J=12.9 Hz, 4H), 2.15 (d, J=11.0 Hz, 1H), 2.11-1.97 (m, 1H), 1.88 (d, J=12.8 Hz, 2H), 1.48 (s, 8H), 1.38 (d, J=11.6 Hz, 2H), 1.00 (t, J=7.1 Hz, 3H).

LCMS: C₄₆H₅₆FN₁₁O₄S₂ requires: 909.4, found: m/z=910.3 [M+H]⁺.

### (3R)-3-{4-[4-({4-[6-({4-[2-tert-butyl-4-(3-{[ethyl(methyl)sulfamoyl]amino}-2-fluorophenyl)-1,3-thiazol-5-yl]pyrimidin-2-yl}amino)pyridin-3-yl]piperazin-1-yl}methyl)piperidin-1-yl]phenyl}piperidine-2,6-dione (128)

LCMS: C₄₆H₅₆FN₁₁O₄S₂ requires: 909.4, found: m/z=910.2 [M+H]⁺.

### Rac-N-(3-{2-tert-butyl-5-[2-({7-[(1-{2-[(3R)-2,6-dioxopiperidin-3-yl]-1-oxo-1,2-dihydroisoquinolin-6-yl}piperidin-4-yl)methyl]-7-azaspiro[3.5]nonan-2-yl}amino)pyrimidin-4-yl]-1,3-thiazol-4-yl}-2-fluorophenyl)propane-1-sulfonamide (129)

LCMS: C₄₈H₅₈FN₉O₅S₂ requires: 923.4, found: m/z=924.2 [M+H]⁺.

### N-[3-(2-tert-butyl-5-{2-[(7-{[(1r,4r)-4-{4-[(3RS)-2,6-dioxopiperidin-3-yl]phenoxy}cyclohexyl]methyl}-7-azaspiro[3.5]nonan-2-yl)amino]pyrimidin-4-yl}-1,3-thiazol-4-yl)-2-fluorophenyl]propane-1-sulfonamide (130)

LCMS: C₄₆H₅₈FN₇O₅S₂ requires: 871.4, found: m/z=872.3 [M+H]⁺.

### Rac-N-[3-(2-tert-butyl-5-{2-[(7-{2-[4-(4-{[(3R)-2,6-dioxopiperidin-3-yl]amino}phenyl)piperidin-1-yl]acetyl}-7-azaspiro[3.5]nonan-2-yl)amino]pyrimidin-4-yl}-1,3-thiazol-4-yl)-2-fluorophenyl]propane-1-sulfonamide (131)

LCMS: C₄₆H₅₈FN₉O₅S₂ requires: 899.4, found: m/z=900.3 [M+H]⁺.

### Rac-N-(3-{2-tert-butyl-5-[2-({7-[2-(4-{2-[(3R)-2,6-dioxopiperidin-3-yl]-1,3-dioxo-2,3-dihydro-1H-isoindol-5-yl}piperazin-1-yl)acetyl]-7-azaspiro[3.5]nonan-2-yl}amino)pyrimidin-4-yl]-1,3-thiazol-4-yl}-2-fluorophenyl)propane-1-sulfonamide (132)

LCMS: C₄₇H₅₅FN₁₀O₇S₂ requires: 954.4, found: m/z=955.4 [M+H]⁺.

### N-(3-{2-tert-butyl-5-[2-({7-[(1-{4-[(3R)-2,6-dioxopiperidin-3-yl]phenyl}piperidin-4-yl)methyl]-7-azaspiro[3.5]nonan-2-yl}amino)pyrimidin-4-yl]-1,3-thiazol-4-yl}-2-fluorophenyl)propane-1-sulfonamide (133)

LCMS: C₄₅H₅₇FN₈O₄S₂ requires: 856.4, found: m/z=857.3 [M+H]⁺.

### N-{3-[2-tert-butyl-5-(2-{[(1s,4s)-4-{4-[(1-{5-[(3RS)-2,6-dioxopiperidin-3-yl]pyridin-2-yl}piperidin-4-yl)methyl]piperazin-1-yl}cyclohexyl]amino}pyrimidin-4-yl)-1,3-thiazol-4-yl]-2-fluorophenyl}propane-1-sulfonamide (134)

LCMS: C₄₆H₆₁FN₁₀O₄S₂ requires: 900.4, found: m/z=901.8 [M+H]⁺.

### N-{3-[2-tert-butyl-5-(2-{[(1r,4r)-4-{4-[(1-{5-[(3RS)-2,6-dioxopiperidin-3-yl]pyridin-2-yl}piperidin-4-yl)methyl]piperazin-1-yl}cyclohexyl]amino}pyrimidin-4-yl)-1,3-thiazol-4-yl]-2-fluorophenyl}propane-1-sulfonamide (135)

LCMS: C₄₆H₆₁FN₁₀O₄S₂ requires: 900.4, found: m/z=901.4 [M+H]⁺.

### Rac-N-(3-{2-tert-butyl-5-[2-({1′-[(1-{5-[(3R)-2,6-dioxopiperidin-3-yl]pyridin-2-yl}piperidin-4-yl)methyl]-[1,4′-bipiperidin]-4-yl}amino)pyrimidin-4-yl]-1,3-thiazol-4-yl}-2-fluorophenyl)propane-1-sulfonamide (136)

LCMS: C₄₆H₆₁FN₁₀O₄S₂ requires: 900.4, found: m/z=901.5 [M+H]⁺.

### N-{3-[2-tert-butyl-5-(2-{[(1r,4r)-4-[4-(1-{5-[(3RS)-2,6-dioxopiperidin-3-yl]pyridin-2-yl}piperidine-4-carbonyl)piperazin-1-yl]cyclohexyl]amino}pyrimidin-4-yl)-1,3-thiazol-4-yl]-2-fluorophenyl}propane-1-sulfonamide (137)

LCMS: C₄₆H₅₉FN₁₀O₅S₂ requires: 914.4, found: m/z=915.4 [M+H]⁺.

### N-{3-[2-tert-butyl-5-(2-{[(1s,4s)-4-[4-(1-{5-[(3RS)-2,6-dioxopiperidin-3-yl]pyridin-2-yl}piperidine-4-carbonyl)piperazin-1-yl]cyclohexyl]amino}pyrimidin-4-yl)-1,3-thiazol-4-yl]-2-fluorophenyl}propane-1-sulfonamide (138)

LCMS: C₄₆H₅₉FN₁₀O₅S₂ requires: 914.4, found: m/z=915.3 [M+H]⁺.

### N-{3-[2-tert-butyl-5-(2-{[(1r,4r)-4-({4-[(1-{4-[(3S)-2,6-dioxopiperidin-3-yl]phenyl}piperidin-4-yl)methyl]piperazin-1-yl}methyl)cyclohexyl]amino}pyrimidin-4-yl)-1,3-thiazol-4-yl]-2-fluorophenyl}propane-1-sulfonamide (139)

LCMS: C₄₈H₆₄FN₉O₄S₂ requires: 913.5, found: m/z=914.4 [M+H]⁺.

### Rac-N-[3-(2-tert-butyl-5-{2-[(2-{[1-(1-{4-[(3R)-2,6-dioxopiperidin-3-yl]phenyl}piperidine-4-carbonyl)piperidin-4-yl]methyl}-2-azaspiro[3.3]heptan-6-yl)amino]pyrimidin-4-yl}-1,3-thiazol-4-yl)-2-fluorophenyl]propane-1-sulfonamide (140)

LCMS: C₄₉H₆₂FN₉O₅S₂ requires: 939.4, found: m/z=940.3 [M+H]⁺.

### Rac-N-[3-(2-tert-butyl-5-{2-[(2-{1-[(1-{4-[(3R)-2,6-dioxopiperidin-3-yl]phenyl}piperidin-4-yl)methyl]-4-fluoropiperidine-4-carbonyl}-2-azaspiro[3.3]heptan-6-yl)amino]pyrimidin-4-yl}-1,3-thiazol-4-yl)-2-fluorophenyl]propane-1-sulfonamide (141)

LCMS: C₄₉H₆₁F₂N₉O₅S₂ requires: 957.4, found: m/z=958.2 [M+H]⁺.

### Rac-N-{3-[2-tert-butyl-5-(2-{[2-({1-[(1-{4-[(3R)-2,6-dioxopiperidin-3-yl]phenyl}piperidin-4-yl)methyl]piperidin-4-yl}methyl)-2-azaspiro[3.3]heptan-6-yl]amino}pyrimidin-4-yl)-1,3-thiazol-4-yl]-2-fluorophenyl}propane-1-sulfonamide (142)

¹H NMR (500 MHz, DMSO-d₆) δ 10.79 (s, 1H), 9.76 (d, J=34.2 Hz, 2H), 8.94 (s, 1H), 8.15 (s, 1H), 7.63-7.49 (m, 2H), 7.41 (t, J=7.0 Hz, 1H), 7.34 (t, J=7.8 Hz, 1H), 7.09 (d, J=8.2 Hz, 2H), 6.96 (d, J=8.2 Hz, 2H), 6.23 (d, J=90.8 Hz, 1H), 4.40-4.03 (m, 3H), 3.57 (d, J=11.9 Hz, 2H), 3.23 (s, 2H), 3.16-2.97 (m, 6H), 2.89 (d, J=11.9 Hz, 2H), 2.82-2.57 (m, 3H), 2.24 (d, J=20.9 Hz, 1H), 2.21-2.12 (m, 1H), 2.08 (s, 6H), 2.01 (dq, J=14.9, 6.2, 5.5 Hz, 2H), 1.85 (t, J=17.8 Hz, 6H), 1.69 (p, J=7.5 Hz, 2H), 1.45 (s, 11H), 1.40-1.29 (m, 2H), 0.91 (t, J=7.4 Hz, 3H).

LCMS: C₄₉H₆₄FN₉O₄S₂ requires: 925.5, found: m/z=926.3 [M+H]⁺.

### 5-(4-{[(3R)-3-{[(4-{2-tert-butyl-4-[2-fluoro-3-(propane-1-sulfonamido)phenyl]-1,3-thiazol-5-yl}pyrimidin-2-yl)amino]methyl}pyrrolidin-1-yl]methyl}piperidin-1-yl)-N-[(3S)-2,6-dioxopiperidin-3-yl]pyridine-2-carboxamide (143)

LCMS: C₄₂H₅₃FN₁₀O₅S₂ requires: 860.4, found: m/z=861.2 [M+H]⁺.

### N-[3-(2-tert-butyl-5-{2-[(2-{1-[(1-{4-[(3R)-2,6-dioxopiperidin-3-yl]phenyl}piperidin-4-yl)methyl]piperidin-4-yl}-2-methylpropyl)amino]pyrimidin-4-yl}-1,3-thiazol-4-yl)-2-fluorophenyl]propane-1-sulfonamide (144)

LCMS: C₄₆H₆₁FN₈O₄S₂ requires: 872.4, found: m/z=873.3 [M+H]⁺.

### N-[3-(2-tert-butyl-5-{2-[({1-[(1-{4-[(3R)-2,6-dioxopiperidin-3-yl]phenyl}piperidin-4-yl)methyl]piperidin-4-yl}methyl)amino]pyrimidin-4-yl}-1,3-thiazol-4-yl)-2-fluorophenyl]propane-1-sulfonamide (145)

LCMS: C₄₃H₅₅FN₈O₄S₂ requires: 830.4, found: m/z=831.2 [M+H]⁺.

### 5-{4-[(4-{1-[(4-{2-tert-butyl-4-[2-fluoro-3-(propane-1-sulfonamido)phenyl]-1,3-thiazol-5-yl}pyrimidin-2-yl)amino]-2-methylpropan-2-yl}piperidin-1-yl)methyl]piperidin-1-yl}-N-[(3S)-2,6-dioxopiperidin-3-yl]pyridine-2-carboxamide (146)

LCMS: C₄₆H₆₁FN₁₀O₅S₂ requires: 916.4, found: m/z=917.3 [M+H]⁺.

### 5-{4-[(4-{[(4-{2-tert-butyl-4-[2-fluoro-3-(propane-1-sulfonamido)phenyl]-1,3-thiazol-5-yl}pyrimidin-2-yl)amino]methyl}piperidin-1-yl)methyl]piperidin-1-yl}-N-[(3S)-2,6-dioxopiperidin-3-yl]pyridine-2-carboxamide (147)

LCMS: C₄₃H₅₅FN₁₀O₅S₂ requires: 874.4, found: m/z=875.2 [M+H]⁺.

### Rac-N-[3-(2-tert-butyl-5-{2-[(2-{2-[4-(3-{2-[(3R)-2,6-dioxopiperidin-3-yl]-1,3-dioxo-2,3-dihydro-1H-isoindol-4-yl}propyl)piperazin-1-yl]acetyl}-2-azaspiro[3.3]heptan-6-yl)amino]pyrimidin-4-yl}-1,3-thiazol-4-yl)-2-fluorophenyl]propane-1-sulfonamide (148)

LCMS: C₄₈H₅₇FN₁₀O₇S₂ requires: 968.4, found: m/z=969.2 [M+H]⁺.

### Rac-N-[3-(2-tert-butyl-5-{2-[(4-{1-[2-(4-{2-[(3R)-2,6-dioxopiperidin-3-yl]-1,3-dioxo-2,3-dihydro-1H-isoindol-5-yl}piperazin-1-yl)acetyl]piperidin-4-yl}phenyl)amino]pyrimidin-4-yl}-1,3-thiazol-4-yl)-2-fluorophenyl]propane-1-sulfonamide (149)

LCMS: C₅₀H₅₅FN₁₀O₇S₂ requires: 990.4, found: m/z=991.2 [M+H]⁺.

### Rac-N-{3-[2-tert-butyl-5-(2-{[2-(6-{2-[(3R)-2,6-dioxopiperidin-3-yl]-3-oxo-2,3-dihydro-1H-isoindol-5-yl}hexanoyl)-2-azaspiro[3.3]heptan-6-yl]amino}pyrimidin-4-yl)-1,3-thiazol-4-yl]-2-fluorophenyl}propane-1-sulfonamide (150)

LCMS: C₄₅H₅₃FN₈O₆S₂ requires: 884.4, found: m/z=885.2 [M+H]⁺.

### Rac-N-(3-{2-tert-butyl-5-[2-({4-[1-(6-{2-[(3R)-2,6-dioxopiperidin-3-yl]-3-oxo-2,3-dihydro-1H-isoindol-5-yl}hexanoyl)piperidin-4-yl]phenyl}amino)pyrimidin-4-yl]-1,3-thiazol-4-yl}-2-fluorophenyl)propane-1-sulfonamide (151)

LCMS: C₅₀H₅₇FN₈O₆S₂ requires: 948.4, found: m/z=885.2 [M+H]⁺.

### Rac-N-(3-{2-tert-butyl-5-[2-({2-[2-(2-{2-[(3R)-2,6-dioxopiperidin-3-yl]-1,3-dioxo-2,3-dihydro-1H-isoindol-5-yl}-2,7-diazaspiro[3.5]nonan-7-yl)acetyl]-2-azaspiro[3.3]heptan-6-yl}amino)pyrimidin-4-yl]-1,3-thiazol-4-yl}-2-fluorophenyl)propane-1-sulfonamide (152)

LCMS: C₄₈H₅₅FN₁₀O₇S₂ requires: 966.4, found: m/z=967.2 [M+H]⁺.

### Rac-N-[3-(2-tert-butyl-5-{2-[(4-{1-[2-(2-{2-[(3R)-2,6-dioxopiperidin-3-yl]-1,3-dioxo-2,3-dihydro-1H-isoindol-5-yl}-2,7-diazaspiro[3.5]nonan-7-yl)acetyl]piperidin-4-yl}phenyl)amino]pyrimidin-4-yl}-1,3-thiazol-4-yl)-2-fluorophenyl]propane-1-sulfonamide (153)

LCMS: C₅₃H₅₉FN₁₀O₇S₂ requires: 1030.4, found: m/z=1031.2 [M+H]⁺.

### Rac-N-(3-{2-tert-butyl-5-[2-({2-[1-(1-{2-[(3R)-2,6-dioxopiperidin-3-yl]-1,3-dioxo-2,3-dihydro-1H-isoindol-5-yl}piperidin-4-yl)azetidine-3-carbonyl]-2-azaspiro[3.3]heptan-6-yl}amino)pyrimidin-4-yl]-1,3-thiazol-4-yl}-2-fluorophenyl)propane-1-sulfonamide (154)

LCMS: C₄₈H₅₅FN₁₀O₇S₂ requires: 966.4, found: m/z=967.2 [M+H]⁺.

### Rac-N-(3-{2-tert-butyl-5-[2-({4-[1-(1-{2-[(3R)-2,6-dioxopiperidin-3-yl]-1,3-dioxo-2,3-dihydro-1H-isoindol-5-yl}piperidine-4-carbonyl)piperidin-4-yl]phenyl}amino)pyrimidin-4-yl]-1,3-thiazol-4-yl}-2-fluorophenyl)propane-1-sulfonamide (155)

LCMS: C₅₀H₅₄FN₉O₇S₂ requires: 975.4, found: m/z=976.2 [M+H]⁺.

### Rac-N-(3-{2-tert-butyl-5-[2-({4-[1-(1-{5-[(3R)-2,6-dioxopiperidin-3-yl]pyridin-2-yl}piperidine-4-carbonyl)piperidin-4-yl]phenyl}amino)pyrimidin-4-yl]-1,3-thiazol-4-yl}-2-fluorophenyl)propane-1-sulfonamide (156)

LCMS: C₄₇H₅₄FN₉O₅S₂ requires: 907.4, found: m/z=930.2 [M+Na]⁺.

### Rac-N-(3-{2-tert-butyl-5-[2-({2-[2-(1′-{2-[(3R)-2,6-dioxopiperidin-3-yl]-1,3-dioxo-2,3-dihydro-1H-isoindol-5-yl}-[1,4′-bipiperidin]-4-yl)acetyl]-2-azaspiro[3.3]heptan-6-yl}amino)pyrimidin-4-yl]-1,3-thiazol-4-yl}-2-fluorophenyl)propane-1-sulfonamide (157)

### Rac-N-[3-(2-tert-butyl-5-{2-[(4-{1-[2-(1′-{2-[(3R)-2,6-dioxopiperidin-3-yl]-1,3-dioxo-2,3-dihydro-1H-isoindol-5-yl}-[1,4′-bipiperidin]-4-yl)acetyl]piperidin-4-yl}phenyl)amino]pyrimidin-4-yl}-1,3-thiazol-4-yl)-2-fluorophenyl]propane-1-sulfonamide (158)

LCMS: C₅₆H₆₅FN₁₀O₇S₂ requires: 1072.4, found: m/z=537.1 [M/2+H]⁺.

### Rac-N-(3-{2-tert-butyl-5-[2-({4-[1-(1-{5-[(3R)-2,6-dioxopiperidin-3-yl]pyridin-2-yl}azetidine-3-carbonyl)piperidin-4-yl]phenyl}amino)pyrimidin-4-yl]-1,3-thiazol-4-yl}-2-fluorophenyl)propane-1-sulfonamide (159)

LCMS: C₄₅H₅₀FN₉O₅S₂ requires: 879.3, found: m/z=880.2 [M+H]⁺.

### Rac-N-{3-[2-tert-butyl-5-(2-{[2-(1-{1-[(3R)-2,6-dioxopiperidin-3-yl]-3-methyl-2-oxo-2,3-dihydro-1H-1,3-benzodiazol-4-yl}piperidine-4-carbonyl)-2-azaspiro[3.3]heptan-6-yl]amino}pyrimidin-4-yl)-1,3-thiazol-4-yl]-2-fluorophenyl}propane-1-sulfonamide (160)

LCMS: C₄₅H₅₃FN₁₀O₆S₂ requires: 912.4, found: m/z=913.2 [M+H]⁺.

### Rac-N-(3-{2-tert-butyl-5-[2-({4-[1-(1-{1-[(3R)-2,6-dioxopiperidin-3-yl]-3-methyl-2-oxo-2,3-dihydro-1H-1,3-benzodiazol-4-yl}piperidine-4-carbonyl)piperidin-4-yl]phenyl}amino)pyrimidin-4-yl]-1,3-thiazol-4-yl}-2-fluorophenyl)propane-1-sulfonamide (161)

LCMS: C₅₀H₅₇FN₁₀O₆S₂ requires: 976.4, found: m/z=977.3 [M+H]⁺.

### N-{3-[2-tert-butyl-5-(2-{[4-(1-{2-[(3S)-1-{1-[(3RS)-2,6-dioxopiperidin-3-yl]-3-methyl-2-oxo-2,3-dihydro-1H-1,3-benzodiazol-5-yl}pyrrolidin-3-yl]acetyl}piperidin-4-yl)phenyl]amino}pyrimidin-4-yl)-1,3-thiazol-4-yl]-2-fluorophenyl}propane-1-sulfonamide (162)

LCMS: C₅₀H₅₇FN₁₀O₆S₂ requires: 976.4, found: m/z=977.2 [M+H]⁺.

### Rac-N-(3-{2-tert-butyl-5-[2-({4-[1-(1-{4-[(3R)-2,6-dioxopiperidin-3-yl]pyridin-2-yl}piperidine-4-carbonyl)piperidin-4-yl]phenyl}amino)pyrimidin-4-yl]-1,3-thiazol-4-yl}-2-fluorophenyl)propane-1-sulfonamide (163)

### N-[3-(2-tert-butyl-5-{2-[(4-{1-[(3R)-1-{5-[(3RS)-2,6-dioxopiperidin-3-yl]pyridin-2-yl}pyrrolidine-3-carbonyl]piperidin-4-yl}phenyl)amino]pyrimidin-4-yl}-1,3-thiazol-4-yl)-2-fluorophenyl]propane-1-sulfonamide (164)

LCMS: C₄₆H₅₂FN₉O₅S₂ requires: 893.2, found: m/z=894.2 [M+H]⁺.

### N-{3-[2-tert-butyl-5-(2-{[4-(1-{2-[(3R)-1-{4-[(3RS)-2,6-dioxopiperidin-3-yl]phenyl}pyrrolidin-3-yl]acetyl}piperidin-4-yl)phenyl]amino}pyrimidin-4-yl)-1,3-thiazol-4-yl]-2-fluorophenyl}propane-1-sulfonamide (165)

LCMS: C₄₈H₅₅FN₈O₅S₂ requires: 906.4, found: m/z=907.2 [M+H]⁺.

### N-{3-[2-tert-butyl-5-(2-{[4-(1-{2-[(3S)-1-{4-[(3RS)-2,6-dioxopiperidin-3-yl]phenyl}pyrrolidin-3-yl]acetyl}piperidin-4-yl)phenyl]amino}pyrimidin-4-yl)-1,3-thiazol-4-yl]-2-fluorophenyl}propane-1-sulfonamide (166)

LCMS: C₄₈H₅₇FN₈O₅S₂ requires: 906.4, found: m/z=907.3 [M+H]⁺.

### Rac-N-[3-(2-tert-butyl-5-{2-[(4-{1-[(1-{4-[(3R)-2,6-dioxopiperidin-3-yl]phenyl}piperidin-4-yl)methyl]piperidin-4-yl}phenyl)amino]pyrimidin-4-yl}-1,3-thiazol-4-yl)-2-fluorophenyl]propane-1-sulfonamide (167)

LCMS: C₄₈H₅₇FN₈O₄S₂ requires: 892.4, found: m/z=893.3 [M+H]⁺.

### N-{3-[2-tert-butyl-5-(2-{[4-(1-{[(1r,4r)-4-({5-[(3RS)-2,6-dioxopiperidin-3-yl]pyridin-2-yl}oxy)cyclohexyl]methyl}piperidin-4-yl)phenyl]amino}pyrimidin-4-yl)-1,3-thiazol-4-yl]-2-fluorophenyl}propane-1-sulfonamide (168)

LCMS: C₄₈H₅₇FN₈O₅S₂ requires: 908.4, found: m/z=909.3 [M+H]⁺.

### Rac-5-{4-[(4-{4-[(4-{2-tert-butyl-4-[2-fluoro-3-(propane-1-sulfonamido)phenyl]-1,3-thiazol-5-yl}pyrimidin-2-yl)amino]phenyl}piperidin-1-yl)methyl]piperidin-1-yl}-N-[(3R)-2,6-dioxopiperidin-3-yl]pyridine-2-carboxamide (169)

LCMS: C₄₈H₅₇FN₁₀O₅S₂ requires: 936.4, found: m/z=937.3 [M+H]⁺.

### Rac-4-[4-({6-[(4-{2-tert-butyl-4-[2-fluoro-3-(propane-1-sulfonamido)phenyl]-1,3-thiazol-5-yl}pyrimidin-2-yl)amino]-2-azaspiro[3.3]heptan-2-yl}methyl)piperidin-1-yl]-N-[(3R)-2,6-dioxopiperidin-3-yl]benzamide (170)

LCMS: C₄₄H₅₄FN₉O₅S₂ requires: 871.4, found: m/z=872.3 [M+H]⁺.

### Rac-4-{4-[(4-{4-[(4-{2-tert-butyl-4-[2-fluoro-3-(propane-1-sulfonamido)phenyl]-1,3-thiazol-5-yl}pyrimidin-2-yl)amino]phenyl}piperidin-1-yl)methyl]piperidin-1-yl}-N-[(3R)-2,6-dioxopiperidin-3-yl]benzamide (171)

LCMS: C₄₉H₅₈FN₉O₅S₂ requires: 935.4, found: m/z=936.3 [M+H]⁺.

### N-[3-(2-tert-butyl-5-{2-[(2-{2-[(3S)-1-{5-[(3RS)-2,6-dioxopiperidin-3-yl]pyridin-2-yl}pyrrolidin-3-yl]ethyl}-2-azaspiro[3.3]heptan-6-yl)amino]pyrimidin-4-yl}-1,3-thiazol-4-yl)-2-fluorophenyl]propane-1-sulfonamide (172)

LCMS: C₄₂H₅₂FN₉O₄S₂ requires: 829.4, found: m/z=830.3 [M+H]⁺.

### N-{3-[2-tert-butyl-5-(2-{[4-(1-{2-[(3S)-1-{5-[(3RS)-2,6-dioxopiperidin-3-yl]pyridin-2-yl}pyrrolidin-3-yl]ethyl}piperidin-4-yl)phenyl]amino}pyrimidin-4-yl)-1,3-thiazol-4-yl]-2-fluorophenyl}propane-1-sulfonamide (173)

LCMS: C₄₇H₅₆FN₉O₄S₂ requires: 893.4, found: m/z=894.4 [M+H]⁺.

### Rac-N-[3-(2-tert-butyl-5-{2-[(4-{1-[2-(4-{4-[(3R)-2,6-dioxopiperidin-3-yl]pyridin-2-yl}piperazin-1-yl)acetyl]piperidin-4-yl}phenyl)amino]pyrimidin-4-yl}-1,3-thiazol-4-yl)-2-fluorophenyl]propane-1-sulfonamide (174)

LCMS: C₄₇H₅₅FN₁₀O₅S₂ requires: 922.4, found: m/z=923.4 [M+H]⁺.

### Rac-N-{3-[2-tert-butyl-5-(2-{[4-(1-{1-[(1-{5-[(3R)-2,6-dioxopiperidin-3-yl]pyridin-2-yl}piperidin-4-yl)methyl]piperidine-4-carbonyl}piperidin-4-yl)phenyl]amino}pyrimidin-4-yl)-1,3-thiazol-4-yl]-2-fluorophenyl}propane-1-sulfonamide (175)

LCMS: C₅₃H₆₅FN₁₀O₅S₂ requires: 1004.5, found: m/z=1005.4 [M+H]⁺.

### Rac-N-[3-(2-tert-butyl-5-{2-[(4-{1-[2-({2-[(3R)-2,6-dioxopiperidin-3-yl]-1,3-dioxo-2,3-dihydro-1H-isoindol-5-yl}oxy)acetyl]piperidin-4-yl}phenyl)amino]pyrimidin-4-yl}-1,3-thiazol-4-yl)-2-fluorophenyl]propane-1-sulfonamide (176)

LCMS: C₄₆H₄₇FN₈O₈S₂ requires: 922.3, found: m/z=923.5 [M+H]⁺.

### Rac-N-[3-(2-tert-butyl-5-{2-[(4-{1-[(1-{5-[(3R)-2,6-dioxopiperidin-3-yl]pyridin-3-yl}piperidin-4-yl)methyl]piperidin-4-yl}phenyl)amino]pyrimidin-4-yl}-1,3-thiazol-4-yl)-2-fluorophenyl]propane-1-sulfonamide (177)

¹H NMR (500 MHz, DMSO-d₆) δ 10.87 (s, 1H), 9.67 (s, 1H), 9.15 (s, 2H), 8.34 (d, J=5.3 Hz, 1H), 8.21 (d, J=2.7 Hz, 1H), 7.85 (s, 1H), 7.67-7.52 (m, 3H), 7.47 (t, J=7.0 Hz, 1H), 7.38 (t, J=7.9 Hz, 1H), 7.22 (s, 1H), 7.15 (d, J=8.1 Hz, 2H), 6.43 (d, J=5.1 Hz, 1H), 3.93-3.72 (m, 3H), 3.68-3.55 (m, 5H), 3.18 (d, J=4.7 Hz, 2H), 3.12 (q, J=7.4 Hz, 6H), 2.99 (t, J=7.7 Hz, 2H), 2.83-2.63 (m, 4H), 2.31 (qd, J=12.7, 4.3 Hz, 2H), 2.02 (dq, J=8.5, 5.8, 4.5 Hz, 2H), 1.67 (h, J=7.5 Hz, 2H), 1.48 (s, 9H), 1.29 (d, J=6.8 Hz, 36H), 0.98-0.82 (m, 4H).

### Rac-N-[3-(2-{1-[1-(1-{4-[(3R)-2,6-dioxopiperidin-3-yl]phenyl}piperidine-4-carbonyl)-4-methylpiperidine-4-carbonyl]piperidin-4-yl}-5-(pyrimidin-4-yl)-1,3-thiazol-4-yl)-2-fluorophenyl]propane-1-sulfonamide (178)

LCMS: C₄₅H₅₃FN₈O₆S₂ requires: 884.4, found: m/z=885.2 [M+H]⁺.

### Rac-N-[3-(2-{1-[1-(1-{4-[(3R)-2,6-dioxopiperidin-3-yl]phenyl}piperidine-4-carbonyl)-4-methylpiperidine-4-carbonyl]piperidin-4-yl}-5-{2-[(propan-2-yl)amino]pyrimidin-4-yl}-1,3-thiazol-4-yl)-2-fluorophenyl]propane-1-sulfonamide (179)

¹H NMR (500 MHz, DMSO-d₆) δ 9.72 (s, 1H), 8.11 (d, J=5.2 Hz, 1H), 7.53 (t, J=7.6 Hz, 1H), 7.37 (d, J=7.1 Hz, 1H), 7.31 (t, J=7.9 Hz, 1H), 7.13 (d, J=7.9 Hz, 1H), 7.05 (s, 2H), 6.90 (s, 2H), 6.11 (s, 1H), 4.36 (s, 2H), 3.67 (s, 4H), 3.13 (s, 1H), 3.07-3.00 (m, 2H), 2.75 (s, 3H), 2.48 (s, 6H), 2.14 (d, J=13.9 Hz, 4H), 2.00 (s, 2H), 1.70 (d, J=7.7 Hz, 1H), 1.68 (s, 3H), 1.63 (s, 3H), 1.46 (s, 1H), 1.39 (s, 1H), 1.27 (s, 3H), 1.11 (d, J=6.6 Hz, 6H), 0.91 (t, J=7.4 Hz, 3H). LCMS: C₄₈H₆₀FN₉O₆S₂ requires: 941.4, found: m/z=942.2 [M+H]⁺.

### N-[3-(2-{1-[1-(1-{5-[(3RS)-2,6-dioxopiperidin-3-yl]pyridin-2-yl}piperidine-4-carbonyl)piperidine-4-carbonyl]piperidin-4-yl}-5-(2-{[(2R)-1-hydroxypropan-2-yl]amino}pyrimidin-4-yl)-1,3-thiazol-4-yl)-2-fluorophenyl]propane-1-sulfonamide (180)

LCMS: C₄₆H₅₇FN₁₀O₇S₂ requires: 944.4, found: m/z=945.3 [M+H]⁺.

### Rac-N-[3-(2-{1-[1-(1-{5-[(3R)-2,6-dioxopiperidin-3-yl]pyridin-2-yl}piperidine-4-carbonyl)-4-methylpiperidine-4-carbonyl]piperidin-4-yl}-5-(pyrimidin-4-yl)-1,3-thiazol-4-yl)-2-fluorophenyl]propane-1-sulfonamide (181)

LCMS: C₄₄H₅₂FN₉O₆S₂ requires: 885.3, found: m/z=886.6 [M+H]⁺.

### Rac-N-[3-(2-{1-[1-(1-{5-[(3R)-2,6-dioxopiperidin-3-yl]pyridin-2-yl}piperidine-4-carbonyl)-4-methylpiperidine-4-carbonyl]piperidin-4-yl}-5-(pyridin-4-yl)-1,3-thiazol-4-yl)-2-fluorophenyl]propane-1-sulfonamide (182)

LCMS: C₄₅H₅₃FN₈O₆S₂ requires: 884.4, found: m/z=855.3 [M+H]⁺.

### Rac-(3S)-3-{6-[4-(4-{4-[4-(3-{[ethyl(methyl)sulfamoyl]amino}-2-fluorophenyl)-5-{2-[(propan-2-yl)amino]pyrimidin-4-yl}-1,3-thiazol-2-yl]piperidine-1-carbonyl}piperidine-1-carbonyl)piperidin-1-yl]pyridin-3-yl}piperidine-2,6-dione (183)

LCMS: C₄₆H₅₈FN₁₁O₆S₂ requires: 943.4, found: m/z=944.2 [M+H]⁺.

### Rac-N-(3-{2-[1-(1-{2-[1-(4-{[(3R)-2,6-dioxopiperidin-3-yl]amino}phenyl)piperidin-4-yl]acetyl}piperidine-4-carbonyl)piperidin-4-yl]-5-(pyrimidin-4-yl)-1,3-thiazol-4-yl}-2-fluorophenyl)propane-1-sulfonamide (184)

LCMS: C₄₅H₅₄FN₉O₆S₂ requires: 899.4, found: m/z=900.3 [M+H]⁺.

### Rac-N-{3-[5-(2-aminopyrimidin-4-yl)-2-[4-(1-{2-[1-(4-{[(3R)-2,6-dioxopiperidin-3-yl]amino}phenyl)piperidin-4-yl]acetyl}piperidin-4-yl)phenyl]-1,3-thiazol-4-yl]-2-fluorophenyl}propane-1-sulfonamide (185)

LCMS: C₄₅H₅₀FN₉O₅S₂ requires: 879.3, found: m/z=880.1 [M+H]⁺.

### N-{3-[5-(2-aminopyrimidin-4-yl)-2-(4-{4-[(1-{4-[(3S)-2,6-dioxopiperidin-3-yl]phenyl}piperidin-4-yl)methyl]piperazin-1-yl}phenyl)-1,3-thiazol-4-yl]-2-fluorophenyl}propane-1-sulfonamide (186)

¹H NMR (500 MHz, DMSO-d₆) δ 10.80 (s, 1H), 9.79 (s, 1H), 9.43 (s, 1H), 8.11 (d, J=5.3 Hz, 1H), 7.92 (d, J=8.4 Hz, 2H), 7.59 (t, J=7.6 Hz, 1H), 7.48 (t, J=7.1 Hz, 1H), 7.38 (t, J=7.9 Hz, 1H), 7.16 (d, J=8.7 Hz, 2H), 7.10 (d, J=8.1 Hz, 2H), 6.95 (d, J=39.3 Hz, 4H), 6.13 (d, J=5.3 Hz, 1H), 4.04 (d, J=12.0 Hz, 3H), 3.83-3.58 (m, 6H), 3.29-3.13 (m, 6H), 3.13-3.04 (m, 2H), 2.79 (s, 2H), 2.66 (tt, J=12.1, 5.2 Hz, 1H), 2.22-1.97 (m, 2H), 1.88 (d, J=12.6 Hz, 2H), 1.72 (q, J=7.6 Hz, 2H), 1.39 (d, J=12.7 Hz, 2H), 0.94 (t, J=7.4 Hz, 3H).

LCMS: C₄₃H₄₈FN₉O₄S₂ requires: 837.3, found: m/z=838.3 [M+H]⁺.

### Rac-N-{3-[5-(2-aminopyrimidin-4-yl)-2-(4-{4-[(1-{5-[(3R)-2,6-dioxopiperidin-3-yl]pyridin-2-yl}piperidin-4-yl)methyl]piperazin-1-yl}phenyl)-1,3-thiazol-4-yl]-2-fluorophenyl}propane-1-sulfonamide (187)

¹H NMR (500 MHz, DMSO-d₆) δ 10.87 (s, 1H), 9.79 (s, 1H), 9.44 (s, 1H), 8.10 (d, J=5.2 Hz, 1H), 7.98-7.85 (m, 3H), 7.59 (t, J=7.7 Hz, 1H), 7.48 (t, J=7.0 Hz, 1H), 7.38 (t, J=7.9 Hz, 1H), 7.16 (d, J=8.5 Hz, 2H), 6.85 (s, 2H), 6.12 (d, J=5.2 Hz, 1H), 4.29 (d, J=12.8 Hz, 2H), 4.04 (d, J=12.3 Hz, 2H), 3.65 (d, J=11.2 Hz, 3H), 3.30-3.05 (m, 7H), 2.98 (s, 3H), 2.77-2.61 (m, 2H), 2.37 (s, 1H), 2.33-2.12 (m, 2H), 2.04-1.93 (m, 1H), 1.87 (d, J=12.7 Hz, 2H), 1.72 (q, J=7.6 Hz, 2H), 1.27 (d, J=13.4 Hz, 2H), 0.94 (t, J=7.4 Hz, 3H). LCMS: C₄₂H₄₇FN₁₀O₄S₂ requires: 838.3, found: m/z=839.1 [M+H]⁺.

### Rac-N-{3-[5-(2-aminopyrimidin-4-yl)-2-{4-[4-(1-{5-[(3R)-2,6-dioxopiperidin-3-yl]pyridin-2-yl}piperidine-4-carbonyl)piperazin-1-yl]phenyl}-1,3-thiazol-4-yl]-2-fluorophenyl}propane-1-sulfonamide (188)

LCMS: C₄₂H₄₅FN₁₀O₅S₂ requires: 852.3, found: m/z=853.5 [M+H]⁺.

### Rac-N-{3-[5-(2-aminopyrimidin-4-yl)-2-(4-{4-[2-(1-{5-[(3R)-2,6-dioxopiperidin-3-yl]pyridin-2-yl}piperidin-4-yl)acetyl]piperazin-1-yl}phenyl)-1,3-thiazol-4-yl]-2-fluorophenyl}propane-1-sulfonamide (189)

LCMS: C₄₃H₄₇FN₁₀O₅S₂ requires: 866.3, found: m/z=867.2 [M+H]⁺.

### Rac-N-{3-[5-(2-aminopyrimidin-4-yl)-2-{4-[4-(1-{4-[(3R)-2,6-dioxopiperidin-3-yl]phenyl}piperidine-4-carbonyl)piperazin-1-yl]phenyl}-1,3-thiazol-4-yl]-2-fluorophenyl}propane-1-sulfonamide (190)

LCMS: C₄₃H₄₆FN₉O₅S₂ requires: 851.3, found: m/z=852.1 [M+H]⁺.

### N-{3-[5-(2-aminopyrimidin-4-yl)-2-(4-{4-[(1-{4-[(3R)-2,6-dioxopiperidin-3-yl]phenyl}piperidin-4-yl)methyl]piperazin-1-yl}phenyl)-1,3-thiazol-4-yl]-2-fluorophenyl}propane-1-sulfonamide (191)

¹H NMR (500 MHz, DMSO-d₆) δ 10.78 (s, 1H), 9.78 (s, 1H), 8.08 (d, J=5.2 Hz, 1H), 7.85 (s, 2H), 7.58 (t, J=7.8 Hz, 1H), 7.53-7.43 (m, 1H), 7.37 (t, J=7.8 Hz, 1H), 7.05 (d, J=8.1 Hz, 4H), 6.91 (d, J=7.7 Hz, 2H), 6.78 (s, 2H), 6.10 (d, J=5.2 Hz, 1H), 3.84-3.61 (m, 4H), 3.13-3.03 (m, 2H), 2.84-2.61 (m, 1H), 2.33-1.96 (m, 4H), 1.83 (d, J=12.4 Hz, 2H), 1.72 (h, J=7.5 Hz, 3H), 1.25 (s, 2H), 0.94 (t, J=7.4 Hz, 3H). LCMS: C₄₃H₄₈FN₉O₄S₂ requires: 837.3, found: m/z=838.4 [M+H]⁺.

### N-{3-[5-(2-aminopyrimidin-4-yl)-2-(4-{1-[(1-{4-[(3S)-2,6-dioxopiperidin-3-yl]phenyl}piperidin-4-yl)methyl]piperidin-4-yl}phenyl)-1,3-thiazol-4-yl]-2-fluorophenyl}propane-1-sulfonamide (192)

LCMS: C₄₄H₄₉FN₈O₄S₂ requires: 836.3, found: m/z=837.3 [M+H]⁺.

### Rac-N-{3-[5-(2-aminopyrimidin-4-yl)-2-{1-[1-(1-{5-[(3R)-2,6-dioxopiperidin-3-yl]pyridin-2-yl}-4-methylpiperidine-4-carbonyl)piperidine-4-carbonyl]piperidin-4-yl}-1,3-thiazol-4-yl]-2-fluorophenyl}propane-1-sulfonamide (193)

¹H NMR (500 MHz, DMSO-d₆) δ 10.87 (s, 1H), 9.74 (s, 1H), 8.09 (d, J=5.2 Hz, 1H), 7.90 (s, 1H), 7.62-7.50 (m, 1H), 7.45-7.37 (m, 1H), 7.33 (t, J=7.9 Hz, 1H), 6.80 (s, 2H), 6.11 (d, J=5.2 Hz, 1H), 4.47 (d, J=12.6 Hz, 1H), 4.30 (d, J=12.8 Hz, 2H), 4.11 (d, J=13.4 Hz, 1H), 3.79 (d, J=41.1 Hz, 3H), 3.12-3.02 (m, 2H), 2.95 (d, J=37.3 Hz, 1H), 2.82-2.62 (m, 2H), 2.34-2.06 (m, 6H), 2.02-1.93 (m, 1H), 1.70 (h, J=6.9, 6.2 Hz, 5H), 1.58 (d, J=11.8 Hz, 3H), 1.46 (d, J=12.2 Hz, 2H), 1.28 (s, 3H), 0.93 (t, J=7.4 Hz, 3H). LCMS: C₄₄H₅₃FN₁₀O₆S₂ requires: 900.4, found: m/z=901.2 [M+H]⁺.

### Rac-N-{3-[5-(2-aminopyrimidin-4-yl)-2-(1-{1-[2-(1-{5-[(3R)-2,6-dioxopiperidin-3-yl]pyridin-2-yl}piperidin-4-yl)acetyl]-4-methylpiperidine-4-carbonyl}piperidin-4-yl)-1,3-thiazol-4-yl]-2-fluorophenyl}propane-1-sulfonamide (194)

¹H NMR (500 MHz, DMSO-d₆) δ 10.93 (s, 1H), 9.75 (s, 1H), 8.09 (d, J=5.3 Hz, 1H), 7.87 (s, 1H), 7.55 (t, J=7.5 Hz, 1H), 7.40 (t, J=7.1 Hz, 1H), 7.33 (t, J=7.8 Hz, 1H), 6.87 (s, 2H), 6.12 (d, J=5.3 Hz, 1H), 4.36 (d, J=13.0 Hz, 2H), 4.17 (d, J=13.2 Hz, 3H), 3.46-3.27 (m, 2H), 3.22-2.95 (m, 7H), 2.75-2.62 (m, 2H), 2.28 (t, J=6.3 Hz, 3H), 2.15 (d, J=12.3 Hz, 2H), 2.11-1.93 (m, 3H), 1.81 (d, J=12.7 Hz, 2H), 1.75-1.54 (m, 4H), 1.42 (d, J=12.8 Hz, 2H), 1.26 (d, J=12.5 Hz, 6H), 0.93 (t, J=7.4 Hz, 3H). LCMS: C₄₅H₅₅FN₁₀O₆S₂ requires: 914.4, found: m/z=915.2 [M+H]⁺.

### Rac-N-{3-[5-(2-aminopyrimidin-4-yl)-2-(1-{1-[(1-{1-[(3R)-2,6-dioxopiperidin-3-yl]-3-methyl-2-oxo-2,3-dihydro-1H-1,3-benzodiazol-4-yl}piperidin-4-yl)methyl]piperidine-4-carbonyl}piperidin-4-yl)-1,3-thiazol-4-yl]-2-fluorophenyl}propane-1-sulfonamide (195)

LCMS: C₄₆H₅₆FN₁₁O₆S₂ requires: 941.4, found: m/z=942.2 [M+H]⁺.

### Rac-N-{3-[5-(2-aminopyrimidin-4-yl)-2-{1-[1-(1-{4-[(3R)-2,6-dioxopiperidin-3-yl]phenyl}piperidine-4-carbonyl)-4-methylpiperidine-4-carbonyl]piperidin-4-yl}-1,3-thiazol-4-yl]-2-fluorophenyl}propane-1-sulfonamide (196)

¹H NMR (500 MHz, DMSO-d₆) δ 10.78 (s, 1H), 9.74 (s, 1H), 8.18-7.96 (m, 1H), 7.55 (t, J=7.6 Hz, 1H), 7.41 (t, J=7.0 Hz, 1H), 7.33 (t, J=7.9 Hz, 1H), 7.06 (s, 2H), 6.91 (s, 2H), 6.79 (s, 2H), 6.10 (d, J=5.2 Hz, 1H), 4.37 (s, 2H), 3.69 (s, 3H), 3.20-2.95 (m, 3H), 2.85-2.59 (m, 5H), 2.23-2.07 (m, 4H), 2.02 (dd, J=13.3, 5.3 Hz, 2H), 1.78-1.56 (m, 7H), 1.43 (d, J=33.5 Hz, 1H), 1.28 (s, 3H), 0.93 (t, J=7.4 Hz, 3H). LCMS: C₄₅H₅₄FN₉O₆S₂ requires: 899.4, found: m/z=900.3 [M+H]⁺.

### Rac-N-{3-[5-(2-aminopyrimidin-4-yl)-2-{1-[1-(1-{5-[(3R)-2,6-dioxopiperidin-3-yl]pyridin-2-yl}piperidine-4-carbonyl)-4-methylpiperidine-4-carbonyl]piperidin-4-yl}-1,3-thiazol-4-yl]-2-fluorophenyl}propane-1-sulfonamide (197)

¹H NMR (500 MHz, DMSO-d₆) δ 10.93 (s, 1H), 9.75 (s, 1H), 8.09 (d, J=5.3 Hz, 1H), 7.89 (d, J=2.2 Hz, 1H), 7.82 (s, 1H), 7.56 (t, J=7.7 Hz, 1H), 7.41 (t, J=7.4 Hz, 1H), 7.34 (t, J=7.9 Hz, 1H), 6.89 (s, 2H), 6.12 (d, J=5.3 Hz, 1H), 4.38 (d, J=12.8 Hz, 2H), 4.19 (s, 3H), 3.90 (d, J=12.0 Hz, 2H), 3.76 (s, 12H), 3.71 (s, 1H), 3.21-3.11 (m, 2H), 3.06 (t, J=7.7 Hz, 2H), 2.73-2.65 (m, 1H), 2.28 (d, J=12.1 Hz, 1H), 2.16 (d, J=12.4 Hz, 2H), 2.05-1.96 (m, 2H), 1.78-1.68 (m, 3H), 1.65-1.60 (m, 5H), 1.29 (s, 3H), 0.93 (t, J=7.5 Hz, 3H). LCMS: C₄₄H₅₃FN₁₀O₆S₂ requires: 900.4, found: m/z=901.2 [M+H]⁺.

### Rac-N-(3-{5-[2-(cyclopropylamino)pyrimidin-4-yl]-2-{1-[1-(1-{5-[(3R)-2,6-dioxopiperidin-3-yl]pyridin-2-yl}piperidine-4-carbonyl)piperidine-4-carbonyl]piperidin-4-yl}-1,3-thiazol-4-yl}-2-fluorophenyl)propane-1-sulfonamide (198)

### Rac-N-[3-(2-{1-[1-(1-{4-[(3R)-2,6-dioxopiperidin-3-yl]phenyl}piperidine-4-carbonyl)piperidine-4-carbonyl]piperidin-4-yl}-5-(pyrimidin-4-yl)-1,3-thiazol-4-yl)-2-fluorophenyl]propane-1-sulfonamide (199)

¹H NMR (500 MHz, DMSO-d₆) δ 10.81 (d, J=8.1 Hz, 1H), 9.74 (s, 1H), 9.16 (s, 1H), 8.68 (d, J=5.5 Hz, 1H), 7.58 (t, J=7.7 Hz, 1H), 7.47 (t, J=6.9 Hz, 1H), 7.36 (t, J=7.9 Hz, 1H), 7.31-7.04 (m, 6H), 4.45 (dd, J=35.7, 12.9 Hz, 2H), 4.14 (d, J=13.4 Hz, 1H), 4.03 (d, J=13.1 Hz, 1H), 3.88-3.63 (m, 3H), 3.28-3.11 (m, 1H), 3.06 (dd, J=9.1, 6.2 Hz, 2H), 3.04-2.58 (m, 4H), 2.26-2.11 (m, 1H), 2.06-1.95 (m, 1H), 1.90-1.64 (m, 10H), 1.46-1.28 (m, 1H), 0.93 (t, J=7.4 Hz, 3H). LCMS: C₄₄H₅₁FN₈O₆S₂ requires: 870.3, found: m/z=871.2 [M+H]⁺.

### N-[3-(2-{1-[1-(1-{4-[(3S)-2,6-dioxopiperidin-3-yl]phenyl}piperidine-4-carbonyl)piperidine-4-carbonyl]piperidin-4-yl}-5-(pyridin-4-yl)-1,3-thiazol-4-yl)-2-fluorophenyl]propane-1-sulfonamide (200)

¹H NMR (500 MHz, DMSO-d₆) δ 10.82 (s, 1H), 9.65 (s, 1H), 8.58 (d, J=5.4 Hz, 2H), 7.51 (t, J=7.7 Hz, 1H), 7.43 (t, J=6.9 Hz, 1H), 7.32 (d, J=7.3 Hz, 3H), 7.17 (s, 7H), 4.45 (dd, J=36.2, 12.9 Hz, 2H), 4.14 (d, J=13.4 Hz, 1H), 4.03 (d, J=13.3 Hz, 1H), 3.80 (s, 1H), 3.69 (d, J=11.8 Hz, 2H), 3.29-3.08 (m, 1H), 3.06-2.84 (m, 3H), 2.78 (t, J=12.1 Hz, 1H), 2.73-2.59 (m, 2H), 2.27-2.11 (m, 3H), 2.03 (dt, J=13.1, 4.9 Hz, 1H), 1.91-1.46 (m, 11H), 1.36 (s, 1H), 0.90 (t, J=7.4 Hz, 3H). LCMS: C₄₅H₅₂FN₇O₆S₂ requires: 869.3, found: m/z=870.3 [M+H]⁺.

### Rac-N-{3-[5-(2-aminopyrimidin-4-yl)-2-{4-[1-(1-{5-[(3R)-2,6-dioxopiperidin-3-yl]pyridin-2-yl}piperidine-4-carbonyl)piperidin-4-yl]phenyl}-1,3-thiazol-4-yl]-2-fluorophenyl}propane-1-sulfonamide (201)

LCMS: C₄₃H₄₆FN₉O₅S₂ requires: 851.3, found: m/z=852.4 [M+H]⁺.

### Rac-N-{3-[5-(2-aminopyrimidin-4-yl)-2-[1-({1-[2-(1-{5-[(3R)-2,6-dioxopiperidin-3-yl]pyridin-2-yl}piperidin-4-yl)acetyl]piperidin-4-yl}methyl)piperidin-4-yl]-1,3-thiazol-4-yl]-2-fluorophenyl}propane-1-sulfonamide (202)

LCMS: C₄₄H₅₅FN₁₀O₅S₂ requires: 886.4, found: m/z=887.2 [M+H]⁺.

### Rac-N-{3-[5-(2-aminopyrimidin-4-yl)-2-(1-{1-[2-(1-{5-[(3R)-2,6-dioxopiperidin-3-yl]pyridin-2-yl}piperidin-4-yl)acetyl]piperidine-4-carbonyl}piperidin-4-yl)-1,3-thiazol-4-yl]-2-fluorophenyl}propane-1-sulfonamide (203)

LCMS: C₄₄H₅₃FN₁₀O₆S₂ requires: 900.4, found: m/z=901.2 [M+H]⁺.

### N-{3-[2-(1-{1-[(1-{4-[(3R)-2,6-dioxopiperidin-3-yl]phenyl}piperidin-4-yl)methyl]piperidine-4-carbonyl}piperidin-4-yl)-5-[2-(methylamino)pyrimidin-4-yl]-1,3-thiazol-4-yl]-2-fluorophenyl}propane-1-sulfonamide (204)

LCMS: C₄₅H₅₆FN₉O₅S₂ requires: 885.4, found: m/z=886.2 [M+H]⁺.

### Rac-N-{3-[5-(2-aminopyrimidin-4-yl)-2-(2-{1-[1-(1-{5-[(3R)-2,6-dioxopiperidin-3-yl]pyridin-2-yl}piperidine-4-carbonyl)piperidine-4-carbonyl]piperidin-4-yl}ethyl)-1,3-thiazol-4-yl]-2-fluorophenyl}propane-1-sulfonamide (205)

LCMS: C₄₅H₅₅FN₁₀O₆S₂ requires: 914.4, found: m/z=915.2 [M+H]⁺.

### Rac-N-[3-(2-{1-[1-(1-{5-[(3R)-2,6-dioxopiperidin-3-yl]pyridin-2-yl}piperidine-4-carbonyl)piperidine-4-carbonyl]piperidin-4-yl}-5-{2-[(propan-2-yl)amino]pyrimidin-4-yl}-1,3-thiazol-4-yl)-2-fluorophenyl]propane-1-sulfonamide (206)

LCMS: C₄₆H₅₇FN₁₀O₆S₂ requires: 928.4, found: m/z=929.2 [M+H]⁺.

### Rac-N-{3-[5-(2-aminopyrimidin-4-yl)-2-(4-{1-[2-(1-{5-[(3R)-2,6-dioxopiperidin-3-yl]pyridin-2-yl}piperidin-4-yl)acetyl]piperidin-4-yl}phenyl)-1,3-thiazol-4-yl]-2-fluorophenyl}propane-1-sulfonamide (207)

LCMS: C₄₄H₄₈FN₉O₅S₂ requires: 865.3, found: m/z=866.1 [M+H]⁺.

### Rac-N-{3-[5-(2-aminopyrimidin-4-yl)-2-(4-{1-[(1-{5-[(3R)-2,6-dioxopiperidin-3-yl]pyridin-2-yl}piperidin-4-yl)methyl]piperidin-4-yl}phenyl)-1,3-thiazol-4-yl]-2-fluorophenyl}propane-1-sulfonamide (208)

LCMS: C₄₃H₄₈FN₉O₄S₂ requires: 837.3, found: m/z=838.2 [M+H]⁺.

### Rac-(3S)-3-{6-[4-(4-{4-[5-(2-aminopyrimidin-4-yl)-4-(3-{[ethyl(methyl)sulfamoyl]amino}-2-fluorophenyl)-1,3-thiazol-2-yl]piperidine-1-carbonyl}piperidine-1-carbonyl)piperidin-1-yl]pyridin-3-yl}piperidine-2,6-dione (209)

LCMS: C₄₃H₅₂FN₁₁O₆S₂ requires: 901.4, found: m/z=902.2 [M+H]⁺.

### Rac-N-[3-(2-{1-[1-(1-{5-[(3R)-2,6-dioxopiperidin-3-yl]pyridin-2-yl}piperidine-4-carbonyl)piperidine-4-carbonyl]piperidin-4-yl}-5-(pyridin-4-yl)-1,3-thiazol-4-yl)-2-fluorophenyl]propane-1-sulfonamide (210)

LCMS: C₄₄H₅₁FN₈O₆S₂ requires: 870.3, found: m/z=871.2 [M+H]⁺.

### Rac-N-{3-[2-(1-{1-[(1-{5-[(3R)-2,6-dioxopiperidin-3-yl]pyridin-2-yl}piperidin-4-yl)methyl]piperidine-4-carbonyl}piperidin-4-yl)-5-(pyridin-4-yl)-1,3-thiazol-4-yl]-2-fluorophenyl}propane-1-sulfonamide (211)

LCMS: C₄₄H₅₃FN₈O₅S₂ requires: 856.4, found: m/z=857.3 [M+H]⁺.

### Rac-N-[3-(2-{1-[1-(1-{5-[(3R)-2,6-dioxopiperidin-3-yl]pyridin-2-yl}piperidine-4-carbonyl)piperidine-4-carbonyl]piperidin-4-yl}-5-[2-(methylamino)pyrimidin-4-yl]-1,3-thiazol-4-yl)-2-fluorophenyl]propane-1-sulfonamide (212)

LCMS: C₄₄H₅₃FN₁₀O₆S₂ requires: 900.4, found: m/z=901.2 [M+H]⁺.

### Rac-N-{3-[5-(2-aminopyrimidin-4-yl)-2-(1-{[1-(1-{5-[(3R)-2,6-dioxopiperidin-3-yl]pyridin-2-yl}piperidine-4-carbonyl)piperidin-4-yl]methyl}piperidin-4-yl)-1,3-thiazol-4-yl]-2-fluorophenyl}propane-1-sulfonamide (213)

LCMS: C₄₃H₅₃FN₁₀O₅S₂ requires: 872.4, found: m/z=873.2 [M+H]⁺.

### Rac-N-{3-[5-(2-aminopyrimidin-4-yl)-2-[1-(1-{2-[1-(4-{[(3R)-2,6-dioxopiperidin-3-yl]amino}-2-fluorophenyl)-4-hydroxypiperidin-4-yl]acetyl}azetidin-3-yl)piperidin-4-yl]-1,3-thiazol-4-yl]-2-fluorophenyl}propane-1-sulfonamide (214)

LCMS: C₄₂H₅₀F₂N₁₀O₆S₂ requires: 892.3, found: m/z=893.2 [M+H]⁺.

### Rac-N-{3-[5-(2-aminopyrimidin-4-yl)-2-(1-{1-[2-(1-{4-[(3R)-2,6-dioxopiperidin-3-yl]phenyl}piperidin-4-yl)acetyl]azetidin-3-yl}piperidin-4-yl)-1,3-thiazol-4-yl]-2-fluorophenyl}propane-1-sulfonamide (215)

LCMS: C₄₂H₅₀FN₉O₅S₂ requires: 843.3, found: m/z=844.2 [M+H]⁺.

### Rac-N-{3-[5-(2-aminopyrimidin-4-yl)-2-{1-[1-(1-{4-[(3R)-2,6-dioxopiperidin-3-yl]phenyl}piperidine-4-carbonyl)piperidine-4-carbonyl]piperidin-4-yl}-1,3-thiazol-4-yl]-2-fluorophenyl}propane-1-sulfonamide (216)

LCMS: C₄₄H₅₂FN₉O₆S₂ requires: 885.3, found: m/z=886.4 [M+H]⁺.

### Rac-N-{3-[2-(1-{1-[(1-{5-[(3R)-2,6-dioxopiperidin-3-yl]pyridin-2-yl}piperidin-4-yl)methyl]piperidine-4-carbonyl}piperidin-4-yl)-5-[2-(methylamino)pyrimidin-4-yl]-1,3-thiazol-4-yl]-2-fluorophenyl}propane-1-sulfonamide (217)

LCMS: C₄₄H₅₅FN₁₀O₅S₂ requires: 886.4, found: m/z=887.4 [M+H]⁺.

### Rac-N-{3-[5-(2-aminopyrimidin-4-yl)-2-(1-{1-[(1-{5-[(3R)-2,6-dioxopiperidin-3-yl]pyridin-2-yl}piperidin-4-yl)methyl]piperidine-4-carbonyl}-4-methylpiperidin-4-yl)-1,3-thiazol-4-yl]-2-fluorophenyl}propane-1-sulfonamide (218)

LCMS: C₄₄H₅₅FN₁₀O₅S₂ requires: 886.4, found: m/z=887.2 [M+H]⁺.

### Rac-N-[3-(2-{1-[2-(2-{2-[(3R)-2,6-dioxopiperidin-3-yl]-1,3-dioxo-2,3-dihydro-1H-isoindol-5-yl}-2,7-diazaspiro[3.5]nonan-7-yl)acetyl]piperidin-4-yl}-5-[2-(methylamino)pyrimidin-4-yl]-1,3-thiazol-4-yl)-2-fluorophenyl]propane-1-sulfonamide (219)

LCMS: C₄₄H₄₉FN₁₀O₇S₂ requires: 912.3, found: m/z=913.2 [M+H]⁺.

### Rac-N-[3-(2-{1-[1-(1-{5-[(3R)-2,6-dioxopiperidin-3-yl]pyridin-2-yl}piperidine-4-carbonyl)piperidine-4-carbonyl]piperidin-4-yl}-5-(pyrimidin-4-yl)-1,3-thiazol-4-yl)-2-fluorophenyl]propane-1-sulfonamide (220)

LCMS: C₄₃H₅₀FN₉O₆S₂ requires: 871.3, found: m/z=872.2 [M+H]⁺.

### Rac-N-{3-[2-(1-{1-[(1-{5-[(3R)-2,6-dioxopiperidin-3-yl]pyridin-2-yl}piperidin-4-yl)methyl]piperidine-4-carbonyl}piperidin-4-yl)-5-(pyrimidin-4-yl)-1,3-thiazol-4-yl]-2-fluorophenyl}propane-1-sulfonamide (221)

LCMS: C₄₃H₅₂FN₉O₅S₂ requires: 857.4, found: m/z=858.2 [M+H]⁺.

### N-{3-[5-(2-aminopyrimidin-4-yl)-2-{1-[(1r,4r)-4-({6-[(3RS)-2,6-dioxopiperidin-3-yl]-1,2,3,4-tetrahydroisoquinolin-2-yl}methyl)cyclohexanecarbonyl]piperidin-4-yl}-1,3-thiazol-4-yl]-2-fluorophenyl}propane-1-sulfonamide (222)

LCMS: C₄₃H₅₁FN₈O₅S₂ requires: 842.3, found: m/z=843.6 [M+H]⁺.

### N-{3-[5-(2-aminopyrimidin-4-yl)-2-[1-({1-[(1r,4r)-4-{3-[(3RS)-2,6-dioxopiperidin-3-yl]phenoxy}cyclohexanecarbonyl]piperidin-4-yl}methyl)piperidin-4-yl]-1,3-thiazol-4-yl]-2-fluorophenyl}propane-1-sulfonamide (223)

LCMS: C₄₅H₅₅FN₈O₆S₂ requires: 886.4, found: m/z=887.3 [M+H]⁺.

### N-{3-[5-(2-aminopyrimidin-4-yl)-2-[1-({1-[(1r,4r)-4-{4-[(3RS)-2,6-dioxopiperidin-3-yl]phenoxy}cyclohexanecarbonyl]piperidin-4-yl}methyl)piperidin-4-yl]-1,3-thiazol-4-yl]-2-fluorophenyl}propane-1-sulfonamide (224)

LCMS: C₄₅H₅₅FN₈O₆S₂ requires: 886.4, found: m/z=887.3 [M+H]⁺.

### N-{3-[5-(2-aminopyrimidin-4-yl)-2-{1-[(1-{[(1r,4r)-4-{4-[(3RS)-2,6-dioxopiperidin-3-yl]phenoxy}cyclohexyl]methyl}piperidin-4-yl)methyl]piperidin-4-yl}-1,3-thiazol-4-yl]-2-fluorophenyl}propane-1-sulfonamide (225)

LCMS: C₄₅H₅₇FN₈O₅S₂ requires: 872.4, found: m/z=873.3 [M+H]⁺.

### Rac-N-{3-[5-(2-aminopyrimidin-4-yl)-2-(1-{[1-(2-{6-[(3R)-2,6-dioxopiperidin-3-yl]-1,2,3,4-tetrahydroisoquinolin-2-yl}acetyl)piperidin-4-yl]methyl}piperidin-4-yl)-1,3-thiazol-4-yl]-2-fluorophenyl}propane-1-sulfonamide (226)

LCMS: C₄₃H₅₂FN₉O₅S₂ requires: 857.4, found: m/z=858.2 [M+H]⁺.

### Rac-N-{3-[5-(2-aminopyrimidin-4-yl)-2-(1-{1-[(1-{4-[(3R)-2,6-dioxopiperidin-3-yl]phenyl}piperidin-4-yl)methyl]piperidine-4-carbonyl}-4-methylpiperidin-4-yl)-1,3-thiazol-4-yl]-2-fluorophenyl}propane-1-sulfonamide (227)

LCMS: C₄₅H₅₆FN₉O₅S₂ requires: 885.4, found: m/z=886.2 [M+H]⁺.

### Rac-N-{3-[5-(2-aminopyrimidin-4-yl)-2-(1-{1-[(1-{4-[(3R)-2,6-dioxopiperidin-3-yl]phenyl}piperidin-4-yl)methyl]piperidine-4-carbonyl}piperidin-4-yl)-1,3-thiazol-4-yl]-2-fluorophenyl}propane-1-sulfonamide (228)

LCMS: C₄₄H₅₄FN₉O₅S₂ requires: 871.4, found: m/z=872.2 [M+H]⁺.

### Rac-N-{3-[5-(2-aminopyrimidin-4-yl)-2-(3-{2-[4-(3-{2-[(3R)-2,6-dioxopiperidin-3-yl]-1,3-dioxo-2,3-dihydro-1H-isoindol-4-yl}propyl)piperazin-1-yl]acetyl}-3-azaspiro[5.5]undecan-9-yl)-1,3-thiazol-4-yl]-2-fluorophenyl}propane-1-sulfonamide (229)

LCMS: C₄₈H₅₇FN₁₀O₇S₂ requires: 968.4, found: m/z=969.2 [M+H]⁺.

### Rac-N-{3-[5-(2-aminopyrimidin-4-yl)-2-{2-[2-(4-{2-[(3R)-2,6-dioxopiperidin-3-yl]-1,3-dioxo-2,3-dihydro-1H-isoindol-5-yl}piperazin-1-yl)acetyl]-2-azaspiro[3.5]nonan-7-yl}-1,3-thiazol-4-yl]-2-fluorophenyl}propane-1-sulfonamide (230)

LCMS: C₄₃H₄₇FN₁₀O₇S₂ requires: 898.3, found: m/z=899.1 [M+H]⁺.

### Rac-N-{3-[5-(2-aminopyrimidin-4-yl)-2-{3-[2-(4-{2-[(3R)-2,6-dioxopiperidin-3-yl]-1,3-dioxo-2,3-dihydro-1H-isoindol-5-yl}piperazin-1-yl)acetyl]-3-azaspiro[5.5]undecan-9-yl}-1,3-thiazol-4-yl]-2-fluorophenyl}propane-1-sulfonamide (231)

LCMS: C₄₅H₅₁FN₁₀O₇S₂ requires: 926.3, found: m/z=927.2 [M+H]⁺.

### Rac-N-{3-[5-(2-aminopyrimidin-4-yl)-2-{1-[2-(4-{2-[(3R)-2,6-dioxopiperidin-3-yl]-1,3-dioxo-2,3-dihydro-1H-isoindol-5-yl}piperazin-1-yl)acetyl]-4-methylpiperidin-4-yl}-1,3-thiazol-4-yl]-2-fluorophenyl}propane-1-sulfonamide (232)

LCMS: C₄₁H₄₅FN₁₀O₇S₂ requires: 872.3, found: m/z=873.1 [M+H]⁺.

### Rac-N-{3-[5-(2-aminopyrimidin-4-yl)-2-{1-[2-(4-{2-[(3R)-2,6-dioxopiperidin-3-yl]-1,3-dioxo-2,3-dihydro-1H-isoindol-5-yl}piperazin-1-yl)acetyl]piperidin-4-yl}-1,3-thiazol-4-yl]-2-fluorophenyl}propane-1-sulfonamide (233)

LCMS: C₄₀H₄₃FN₁₀O₇S₂ requires: 858.3, found: m/z=859.1 [M+H]⁺.

### Rac-N-{3-[5-(2-aminopyrimidin-4-yl)-2-[2-(6-{2-[(3R)-2,6-dioxopiperidin-3-yl]-3-oxo-2,3-dihydro-1H-isoindol-5-yl}hexanoyl)-2-azaspiro[3.5]nonan-7-yl]-1,3-thiazol-4-yl]-2-fluorophenyl}propane-1-sulfonamide (234)

LCMS: C₄₃H₄₉FN₈O₆S₂ requires: 856.3, found: m/z=857.1 [M+H]⁺.

### Rac-N-{3-[5-(2-aminopyrimidin-4-yl)-2-[3-(6-{2-[(3R)-2,6-dioxopiperidin-3-yl]-3-oxo-2,3-dihydro-1H-isoindol-5-yl}hexanoyl)-3-azaspiro[5.5]undecan-9-yl]-1,3-thiazol-4-yl]-2-fluorophenyl}propane-1-sulfonamide (235)

LCMS: C₄₅H₅₃FN₈O₆S₂ requires: 884.4, found: m/z=885.2 [M+H]⁺.

### Rac-N-{3-[5-(2-aminopyrimidin-4-yl)-2-{2-[2-(2-{2-[(3R)-2,6-dioxopiperidin-3-yl]-1,3-dioxo-2,3-dihydro-1H-isoindol-5-yl}-2,7-diazaspiro[3.5]nonan-7-yl)acetyl]-2-azaspiro[3.5]nonan-7-yl}-1,3-thiazol-4-yl]-2-fluorophenyl}propane-1-sulfonamide (236)

LCMS: C₄₆H₅₁FN₁₀O₇S₂ requires: 937.3, found: m/z=939.2 [M+H]⁺.

### Rac-N-{3-[5-(2-aminopyrimidin-4-yl)-2-{3-[2-(2-{2-[(3R)-2,6-dioxopiperidin-3-yl]-1,3-dioxo-2,3-dihydro-1H-isoindol-5-yl}-2,7-diazaspiro[3.5]nonan-7-yl)acetyl]-3-azaspiro[5.5]undecan-9-yl}-1,3-thiazol-4-yl]-2-fluorophenyl}propane-1-sulfonamide (237)

LCMS: C₄₈H₅₅FN₁₀O₇S₂ requires: 966.4, found: m/z=967.2 [M+H]⁺.

### Rac-N-{3-[5-(2-aminopyrimidin-4-yl)-2-{1-[2-(2-{2-[(3R)-2,6-dioxopiperidin-3-yl]-1,3-dioxo-2,3-dihydro-1H-isoindol-5-yl}-2,7-diazaspiro[3.5]nonan-7-yl)acetyl]-4-methylpiperidin-4-yl}-1,3-thiazol-4-yl]-2-fluorophenyl}propane-1-sulfonamide (238)

LCMS: C₄₄H₄₉FN₁₀O₇S₂ requires: 912.3, found: m/z=913.1 [M+H]⁺.

### Rac-N-{3-[5-(2-aminopyrimidin-4-yl)-2-{1-[2-(2-{2-[(3R)-2,6-dioxopiperidin-3-yl]-1,3-dioxo-2,3-dihydro-1H-isoindol-5-yl}-2,7-diazaspiro[3.5]nonan-7-yl)acetyl]piperidin-4-yl}-1,3-thiazol-4-yl]-2-fluorophenyl}propane-1-sulfonamide (239)

¹H NMR (500 MHz, DMSO-d₆) δ 11.08 (s, 1H), 9.74 (s, 1H), 9.55 (s, 1H), 8.10 (d, J=5.2 Hz, 1H), 7.69 (d, J=8.2 Hz, 1H), 7.56 (td, J=7.8, 1.9 Hz, 1H), 7.43-7.37 (m, 1H), 7.34 (t, J=7.8 Hz, 1H), 6.94-6.74 (m, 3H), 6.68 (dd, J=8.4, 2.2 Hz, 1H), 6.12 (d, J=5.2 Hz, 1H), 5.07 (dd, J=12.7, 5.5 Hz, 1H), 4.51-4.25 (m, 3H), 3.44 (tt, J=7.3, 3.6 Hz, 3H), 3.28 (t, J=12.5 Hz, 1H), 3.17-3.00 (m, 3H), 3.00-2.80 (m, 2H), 2.67-2.56 (m, 2H), 2.27-2.11 (m, 4H), 2.11-1.97 (m, 2H), 1.91-1.76 (m, 1H), 1.80-1.57 (m, 3H), 0.93 (t, J=7.4 Hz, 3H). LCMS: C₄₃H₄₇FN₁₀O₇S₂ requires: 898.3, found: m/z=900.0 [M+H]⁺.

### Rac-N-{3-[5-(2-aminopyrimidin-4-yl)-2-(2-{1-[2-(2-{2-[(3R)-2,6-dioxopiperidin-3-yl]-1,3-dioxo-2,3-dihydro-1H-isoindol-5-yl}-2,7-diazaspiro[3.5]nonan-7-yl)acetyl]piperidin-4-yl}propan-2-yl)-1,3-thiazol-4-yl]-2-fluorophenyl}propane-1-sulfonamide (240)

LCMS: C₄₆H₅₃FN₁₀O₇S₂ requires: 940.4, found: m/z=941.2 [M+H]⁺.

### Rac-N-{3-[5-(2-aminopyrimidin-4-yl)-2-{2-[1-(1-{2-[(3R)-2,6-dioxopiperidin-3-yl]-1,3-dioxo-2,3-dihydro-1H-isoindol-5-yl}piperidin-4-yl)azetidine-3-carbonyl]-2-azaspiro[3.5]nonan-7-yl}-1,3-thiazol-4-yl]-2-fluorophenyl}propane-1-sulfonamide (241)

LCMS: C₄₆H₅₁FN₁₀O₇S₂ requires: 938.3, found: m/z=939.0 [M+H]⁺.

### Rac-N-{3-[5-(2-aminopyrimidin-4-yl)-2-{1-[1-(1-{2-[(3R)-2,6-dioxopiperidin-3-yl]-1,3-dioxo-2,3-dihydro-1H-isoindol-5-yl}piperidin-4-yl)azetidine-3-carbonyl]piperidin-4-yl}-1,3-thiazol-4-yl]-2-fluorophenyl}propane-1-sulfonamide (242)

LCMS: C₄₃H₄₇FN₁₀O₇S₂ requires: 898.3, found: m/z=899.1 [M+H]⁺.

### Rac-N-{3-[5-(2-aminopyrimidin-4-yl)-2-(2-{1-[1-(1-{2-[(3R)-2,6-dioxopiperidin-3-yl]-1,3-dioxo-2,3-dihydro-1H-isoindol-5-yl}piperidin-4-yl)azetidine-3-carbonyl]piperidin-4-yl}propan-2-yl)-1,3-thiazol-4-yl]-2-fluorophenyl}propane-1-sulfonamide (243)

LCMS: C₄₆H₅₃FN₁₀O₇S₂ requires: 940.4, found: m/z=941.2 [M+H]⁺.

### Rac-N-{3-[5-(2-aminopyrimidin-4-yl)-2-[2-(1-{2-[(3R)-2,6-dioxopiperidin-3-yl]-1,3-dioxo-2,3-dihydro-1H-isoindol-5-yl}piperidine-4-carbonyl)-2-azaspiro[3.5]nonan-7-yl]-1,3-thiazol-4-yl]-2-fluorophenyl}propane-1-sulfonamide (244)

LCMS: C₄₃H₄₆FN₉O₇S₂ requires: 883.3, found: m/z=884.2 [M+H]⁺.

### Rac-N-{3-[5-(2-aminopyrimidin-4-yl)-2-[3-(1-{2-[(3R)-2,6-dioxopiperidin-3-yl]-1,3-dioxo-2,3-dihydro-1H-isoindol-5-yl}piperidine-4-carbonyl)-3-azaspiro[5.5]undecan-9-yl]-1,3-thiazol-4-yl]-2-fluorophenyl}propane-1-sulfonamide (245)

LCMS: C₄₅H₅₀FN₉O₇S₂ requires: 911.3, found: m/z=912.2 [M+H]⁺.

### Rac-N-{3-[5-(2-aminopyrimidin-4-yl)-2-[3-(1-{5-[(3R)-2,6-dioxopiperidin-3-yl]pyridin-2-yl}piperidine-4-carbonyl)-3-azaspiro[5.5]undecan-9-yl]-1,3-thiazol-4-yl]-2-fluorophenyl}propane-1-sulfonamide (246)

LCMS: C₄₂H₅₀FN₉O₅S₂ requires: 843.3, found: m/z=422.6 [M/2]+.

### Rac-N-{3-[5-(2-aminopyrimidin-4-yl)-2-{2-[2-(1′-{2-[(3R)-2,6-dioxopiperidin-3-yl]-1,3-dioxo-2,3-dihydro-1H-isoindol-5-yl}-[1,4′-bipiperidin]-4-yl)acetyl]-2-azaspiro[3.5]nonan-7-yl}-1,3-thiazol-4-yl]-2-fluorophenyl}propane-1-sulfonamide (247)

LCMS: C₄₉H₅₇FN₁₀O₇S₂ requires: 980.4, found: m/z=981.6 [M+H]⁺.

### Rac-N-{3-[5-(2-aminopyrimidin-4-yl)-2-{3-[2-(1′-{2-[(3R)-2,6-dioxopiperidin-3-yl]-1,3-dioxo-2,3-dihydro-1H-isoindol-5-yl}-[1,4′-bipiperidin]-4-yl)acetyl]-3-azaspiro[5.5]undecan-9-yl}-1,3-thiazol-4-yl]-2-fluorophenyl}propane-1-sulfonamide (248)

LCMS: C₅₁H₆₁FN₁₀O₇S₂ requires: 1008.4, found: m/z=1009.2 [M+H]⁺.

### Rac-N-{3-[5-(2-aminopyrimidin-4-yl)-2-{1-[2-(1′-{2-[(3R)-2,6-dioxopiperidin-3-yl]-1,3-dioxo-2,3-dihydro-1H-isoindol-5-yl}-[1,4′-bipiperidin]-4-yl)acetyl]-4-methylpiperidin-4-yl}-1,3-thiazol-4-yl]-2-fluorophenyl}propane-1-sulfonamide (249)

LCMS: C₄₇H₅₅FN₁₀O₇S₂ requires: 954.4, found: m/z=955.2 [M+H]⁺.

### Rac-N-{3-[5-(2-aminopyrimidin-4-yl)-2-{1-[2-(1′-{2-[(3R)-2,6-dioxopiperidin-3-yl]-1,3-dioxo-2,3-dihydro-1H-isoindol-5-yl}-[1,4′-bipiperidin]-4-yl)acetyl]piperidin-4-yl}-1,3-thiazol-4-yl]-2-fluorophenyl}propane-1-sulfonamide (250)

LCMS: C₄₆H₅₃FN₁₀O₇S₂ requires: 940.4, found: m/z=941.2 [M+H]⁺.

### Rac-N-{3-[5-(2-aminopyrimidin-4-yl)-2-[3-(1-{1-[(3R)-2,6-dioxopiperidin-3-yl]-3-methyl-2-oxo-2,3-dihydro-1H-1,3-benzodiazol-4-yl}piperidine-4-carbonyl)-3-azaspiro[5.5]undecan-9-yl]-1,3-thiazol-4-yl]-2-fluorophenyl}propane-1-sulfonamide (251)

LCMS: C₄₅H₅₃FN₁₀O₆S₂ requires: 912.4, found: m/z=913.3 [M+H]⁺.

### Rac-5-(4-{9-[5-(2-aminopyrimidin-4-yl)-4-[2-fluoro-3-(propane-1-sulfonamido)phenyl]-1,3-thiazol-2-yl]-3-azaspiro[5.5]undecane-3-carbonyl}piperidin-1-yl)-N-[(3R)-2,6-dioxopiperidin-3-yl]pyridine-2-carboxamide (252)

LCMS: C₄₃H₅₁FN₁₀O₆S₂ requires: 886.3, found: m/z=887.2 [M+H]⁺.

### N-{3-[5-(2-aminopyrimidin-4-yl)-2-(3-{2-[(3R)-1-{4-[(3RS)-2,6-dioxopiperidin-3-yl]phenyl}pyrrolidin-3-yl]acetyl}-3-azaspiro[5.5]undecan-9-yl)-1,3-thiazol-4-yl]-2-fluorophenyl}propane-1-sulfonamide (253)

LCMS: C₄₃H₅₁FN₈O₅S₂ requires: 842.3, found: m/z=843.2 [M+H]⁺.

### Rac-N-{3-[5-(2-aminopyrimidin-4-yl)-2-{1-[1-(1-{5-[(3R)-2,6-dioxopiperidin-3-yl]pyridin-2-yl}piperidine-4-carbonyl)piperidine-4-carbonyl]piperidin-4-yl}-1,3-thiazol-4-yl]-2-fluorophenyl}propane-1-sulfonamide (254)

¹H NMR (500 MHz, DMSO-d₆) δ 10.81 (s, 1H), 9.74 (s, 1H), 8.08 (d, J=5.2 Hz, 1H), 7.96 (d, J=2.4 Hz, 1H), 7.59-7.49 (m, 1H), 7.46-7.36 (m, 2H), 7.33 (t, J=7.9 Hz, 1H), 6.88-6.72 (m, 3H), 6.11 (d, J=5.2 Hz, 1H), 4.43 (dd, J=39.3, 12.8 Hz, 2H), 4.28 (d, J=12.6 Hz, 2H), 4.08 (dd, J=36.0, 13.4 Hz, 2H), 3.74 (dd, J=12.2, 4.9 Hz, 1H), 3.29-3.19 (m, 1H), 3.14 (t, J=12.6 Hz, 1H), 3.09-3.02 (m, 2H), 3.02-2.83 (m, 4H), 2.82-2.59 (m, 3H), 2.37 (d, J=3.0 Hz, 1H), 2.27-2.07 (m, 3H), 2.06-1.91 (m, 1H), 1.70 (td, J=16.4, 15.6, 8.1 Hz, 7H), 1.51 (d, J=32.9 Hz, 1H), 1.35 (s, 1H), 0.93 (t, J=7.4 Hz, 3H). LCMS: C₄₃H₅₁FN₁₀O₆S₂ requires: 886.3, found: m/z=887.2 [M+H]⁺.

### 4-({4-[5-(2-aminopyrimidin-4-yl)-4-[2-fluoro-3-(propane-1-sulfonamido)phenyl]-1,3-thiazol-2-yl]piperidin-1-yl}methyl)-N-[(3R)-2,6-dioxopiperidin-3-yl]benzamide (255)

LCMS: C₃₄H₃₇FN₈O₅S₂ requires: 720.2, found: m/z=721.2 [M+H]⁺.

### Rac-N-{3-[5-(2-aminopyrimidin-4-yl)-2-{3-[(1-{4-[(3R)-2,6-dioxopiperidin-3-yl]phenyl}piperidin-4-yl)methyl]-3-azaspiro[5.5]undecan-9-yl}-1,3-thiazol-4-yl]-2-fluorophenyl}propane-1-sulfonamide (256)

LCMS: C₄₃H₅₃FN₈O₄S₂ requires: 828.4, found: m/z=829.3 [M+H]⁺.

### N-{3-[5-(2-aminopyrimidin-4-yl)-2-(3-{[(1r,4r)-4-({5-[(3RS)-2,6-dioxopiperidin-3-yl]pyridin-2-yl}oxy)cyclohexyl]methyl}-3-azaspiro[5.5]undecan-9-yl)-1,3-thiazol-4-yl]-2-fluorophenyl}propane-1-sulfonamide (257)

LCMS: C₄₃H₅₃FN₈O₅S₂ requires: 844.3, found: m/z=845.3 [M+H]⁺.

### Rac-N-{3-[5-(2-aminopyrimidin-4-yl)-2-{3-[(1-{2-[(3R)-2,6-dioxopiperidin-3-yl]-1-oxo-2,3-dihydro-1H-isoindol-5-yl}piperidin-4-yl)methyl]-3-azaspiro[5.5]undecan-9-yl}-1,3-thiazol-4-yl]-2-fluorophenyl}propane-1-sulfonamide (258)

LCMS: C₄₅H₅₄FN₉O₅S₂ requires: 883.4, found: m/z=845.3 [M+H]⁺.

### N-{3-[5-(2-aminopyrimidin-4-yl)-2-(3-{2-[(3S)-1-{5-[(3RS)-2,6-dioxopiperidin-3-yl]pyridin-2-yl}pyrrolidin-3-yl]ethyl}-3-azaspiro[5.5]undecan-9-yl)-1,3-thiazol-4-yl]-2-fluorophenyl}propane-1-sulfonamide (259)

LCMS: C₄₂H₅₂FN₉O₄S₂ requires: 829.4, found: m/z=830.3 [M+H]⁺.

### Rac-N-{3-[5-(2-aminopyrimidin-4-yl)-2-{3-[(1-{5-[(3R)-2,6-dioxopiperidin-3-yl]pyridin-2-yl}piperidin-4-yl)methyl]-3-azaspiro[5.5]undecan-9-yl}-1,3-thiazol-4-yl]-2-fluorophenyl}propane-1-sulfonamide (260)

LCMS: C₄₂H₅₂FN₉O₄S₂ requires: 829.4, found: m/z=830.5 [M+H]⁺.

### Rac-N-{3-[5-(2-aminopyrimidin-4-yl)-2-(1-{1-[(1-{5-[(3R)-2,6-dioxopiperidin-3-yl]pyridin-2-yl}piperidin-4-yl)methyl]piperidine-4-carbonyl}piperidin-4-yl)-1,3-thiazol-4-yl]-2-fluorophenyl}propane-1-sulfonamide (261)

LCMS: C₄₃H₅₃FN₁₀O₅S₂ requires: 872.4, found: m/z=873.7 [M+H]⁺.

### Rac-2-[(3R)-2,6-dioxopiperidin-3-yl]-5-(3-{[3-(4-{4-[(1Z)-1-(hydroxyimino)-2,3-dihydro-1H-inden-5-yl]-3-(pyridin-4-yl)-1H-pyrazol-1-yl}phenyl)azetidin-1-yl]methyl}azetidin-1-yl)-2,3-dihydro-1H-isoindole-1,3-dione (262)

(R)—N-(5-chloro-3-(1-(4-(4-((1-(5-(2,4-dioxotetrahydropyrimidin-1(2H)-yl)pyridin-2-yl)piperidin-4-yl)methyl)piperazin-1-yl)phenyl)-3-(pyridin-4-yl)-1H-pyrazol-4-yl)-2-fluorophenyl)-3-fluoropyrrolidine-1-sulfonamide (263)

LCMS: C₄₃H₄₆ClF₂N₁₁O₄S requires: 885.3, found: m/z=886.2 [M+H]⁺.

### (R)—N-(5-chloro-3-(1-(4-(4-((1-(5-((R)-2,6-dioxopiperidin-3-yl)pyridin-2-yl)piperidin-4-yl)methyl)piperazin-1-yl)phenyl)-3-(pyridin-4-yl)-1H-pyrazol-4-yl)-2-fluorophenyl)-3-fluoropyrrolidine-1-sulfonamide (264)

LCMS: C₄₄H₄₇ClF₂N₁₀O₄S requires: 884.3, found: m/z=885.2 [M+H]⁺.

### Rac-(R)—N-(5-chloro-3-(1-(4-(4-((1-(5-(2,6-dioxopiperidin-3-yl)pyridin-2-yl)piperidin-4-yl)methyl)piperazin-1-yl)phenyl)-3-(pyridin-4-yl)-1H-pyrazol-4-yl)-2-fluorophenyl)pyrrolidine-1-sulfonamide (265)

¹H NMR (500 MHz, DMSO) δ 10.83 (s, 1H), 9.94 (s, 1H), 8.79 (s, 1H), 8.64-8.53 (m, 2H), 7.96 (d, J=2.5 Hz, 1H), 7.85 (d, J=8.8 Hz, 2H), 7.53 (dd, J=6.4, 2.7 Hz, 1H), 7.48-7.38 (m, 4H), 7.20 (d, J=8.7 Hz, 2H), 6.90 (s, 1H), 4.31 (d, J=12.9 Hz, 2H), 3.91 (d, J=12.5 Hz, 2H), 3.76 (dd, J=12.1, 4.8 Hz, 1H), 3.64 (d, J=11.5 Hz, 2H), 3.20-3.07 (m, 8H), 2.89 (t, J=12.6 Hz, 2H), 2.70 (ddt, J=17.4, 12.4, 5.6 Hz, 1H), 2.54 (d, J=4.2 Hz, 1H), 2.20 (tt, J=12.5, 6.4 Hz, 2H), 2.03-1.94 (m, 1H), 1.89 (d, J=12.6 Hz, 2H), 1.78-1.67 (m, 4H), 1.25 (d, J=11.7 Hz, 2H).

LCMS: C₄₄H₄₈ClFN₁₀O₄S requires: 866.3, found: m/z=867.3 [M+H]⁺.

### Rac-(R)—N-(5-chloro-3-(1-(4-(4-((1-(5-(2,6-dioxopiperidin-3-yl)pyridin-2-yl)piperidin-4-yl)methyl)piperazin-1-yl)-2-fluorophenyl)-3-(pyridin-4-yl)-1H-pyrazol-4-yl)-2-fluorophenyl)pyrrolidine-1-sulfonamide (266)

¹H NMR (500 MHz, DMSO) δ 10.73 (s, 1H), 9.86 (s, 1H), 8.51-8.47 (m, 2H), 8.37 (d, J=2.0 Hz, 1H), 7.87 (d, J=2.5 Hz, 1H), 7.60 (t, J=9.0 Hz, 1H), 7.44 (dd, J=6.4, 2.7 Hz, 1H), 7.36-7.25 (m, 4H), 6.98 (d, J=14.3 Hz, 1H), 6.88 (d, J=9.1 Hz, 1H), 6.73 (d, J=8.9 Hz, 1H), 4.20 (d, J=12.8 Hz, 2H), 3.66 (dd, J=12.1, 4.9 Hz, 1H), 3.27 (s, 10H), 3.11-3.04 (m, 4H), 2.73 (t, J=12.4 Hz, 2H), 2.61 (td, J=12.2, 6.2 Hz, 1H), 2.46 (t, J=4.1 Hz, 1H), 2.11 (td, J=12.5, 8.3 Hz, 1H), 1.90 (dq, J=13.4, 4.7 Hz, 1H), 1.73 (d, J=12.9 Hz, 2H), 1.70-1.62 (m, 4H), 1.05 (d, J=12.5 Hz, 2H).

### Rac-(R)—N-(3-(1-(4-(4-((1-(5-(2,6-dioxopiperidin-3-yl)pyridin-2-yl)piperidin-4-yl)methyl)piperazin-1-yl)phenyl)-3-(pyridin-4-yl)-1H-pyrazol-4-yl)-2,5-difluorophenyl)pyrrolidine-1-sulfonamide (267)

LCMS: C₄₄H₄₈F₂N₁₀O₄S requires: 850.4, found: m/z=851.3 [M+H]⁺.

### N-(5-chloro-3-(1-(4-((R)-4-((1-(5-((RS)-2,6-dioxopiperidin-3-yl)pyridin-2-yl)piperidin-4-yl)methyl)-3-methylpiperazin-1-yl)phenyl)-3-(pyridin-4-yl)-1H-pyrazol-4-yl)-2-fluorophenyl)pyrrolidine-1-sulfonamide (268)

LCMS: C₄₅H₅₀ClFN₁₀O₄S requires: 880.3, found: m/z=881.2 [M+H]⁺.

### (R)—N-(3-(1-(4-(4-((1-(4-(2,6-dioxopiperidin-3-yl)-2-fluorophenyl)piperidin-4-yl)methyl)piperazin-1-yl)-2-fluorophenyl)-3-(pyridin-4-yl)-1H-pyrazol-4-yl)-2,5-difluorophenyl)cyclopentanesulfonamide (269)

LCMS C₄₆H₄₈F₄N₈O₄S requires: 884.4, found: m/z=885.2 [M+H]⁺.

### Rac-(R)—N-(3-(1-(4-(4-((1-(5-(2,6-dioxopiperidin-3-yl)pyridin-2-yl)piperidin-4-yl)methyl)piperazin-1-yl)-3-fluorophenyl)-3-(pyridin-4-yl)-1H-pyrazol-4-yl)-2,5-difluorophenyl)cyclopentanesulfonamide (270)

LCMS C₄₅H₄₈F₃N₉O₄S requires: 867.4, found: m/z=868.2 [M+H]⁺.

### Rac-(R)—N-(5-chloro-3-(1-(4-(4-((1-(5-(2,6-dioxopiperidin-3-yl)pyridin-2-yl)piperidin-4-yl)methyl)piperazin-1-yl)-3-fluorophenyl)-3-(pyridin-4-yl)-1H-pyrazol-4-yl)-2-fluorophenyl)cyclopentanesulfonamide (271)

LCMS C₄₅H₄₈ClF₂N₉O₄S requires: 883.3 found m/z=884.6 [M+H]⁺.

### (R)—N-(5-chloro-3-(1-(4-(4-((1-(4-(2,6-dioxopiperidin-3-yl)-2-fluorophenyl)piperidin-4-yl)methyl)piperazin-1-yl)-3-fluorophenyl)-3-(pyridin-4-yl)-1H-pyrazol-4-yl)-2-fluorophenyl)cyclopentanesulfonamide (272)

LCMS C₄₆H₄₈ClF₃N₈O₄S requires: 900.3, found m/z=901.3 [M+H]⁺.

### (R)—N-(5-chloro-3-(1-(4-(4-((1-(4-(2,6-dioxopiperidin-3-yl)-2-fluorophenyl)piperidin-4-yl)methyl)-1,4-diazepan-1-yl)-2-fluorophenyl)-3-(pyridin-4-yl)-1H-pyrazol-4-yl)-2-fluorophenyl)pyrrolidine-1-sulfonamide (273)

LCMS C₄₆H₄₉ClF₃N₉O₄S requires: 915.3, found m/z=916.5 [M+H]⁺.

### (R)—N-(3-(1-(4-(4-((1-(4-(2,6-dioxopiperidin-3-yl)-2-fluorophenyl)piperidin-4-yl)methyl)-1,4-diazepan-1-yl)-2-fluorophenyl)-3-(pyridin-4-yl)-1H-pyrazol-4-yl)-2,5-difluorophenyl)pyrrolidine-1-sulfonamide (274)

LCMS C₄₆H₄₉F₄N₉O₄S requires: 899.4, found: m/z=900.7 [M+H]⁺.

### N-[5-chloro-3-(1-{4-[(3R)-4-[(1-{4-[(3R)-2,6-dioxopiperidin-3-yl]phenyl}piperidin-4-yl)methyl]-3-methylpiperazin-1-yl]phenyl}-3-(pyridin-4-yl)-1H-pyrazol-4-yl)-2-fluorophenyl]propane-1-sulfonamide (275)

LCMS: C₄₅H₅₀ClFN₈O₄S requires: 852.3, found: m/z=853.2 [M+H]⁺.

### N-[5-chloro-3-(1-{4-[(3R)-4-[(1-{5-[(3RS)-2,6-dioxopiperidin-3-yl]pyridin-2-yl}piperidin-4-yl)methyl]-3-methylpiperazin-1-yl]phenyl}-3-(pyridin-4-yl)-1H-pyrazol-4-yl)-2-fluorophenyl]propane-1-sulfonamide (276)

LCMS: C₄₄H₄₉ClFN₉O₄S requires: 853.3, found: m/z=854.2 [M+H]⁺.

### N-[5-chloro-3-(1-{4-[(3R)-4-({1-[4-(2,4-dioxo-1,3-diazinan-1-yl)phenyl]piperidin-4-yl}methyl)-3-methylpiperazin-1-yl]phenyl}-3-(pyridin-4-yl)-1H-pyrazol-4-yl)-2-fluorophenyl]propane-1-sulfonamide (277)

LCMS: C₄₄H₄₉ClFN₉O₄S requires: 853.3, found: m/z=854.2 [M+H]⁺.

### N-[5-chloro-3-(1-{4-[(3S)-4-[(1-{4-[(3R)-2,6-dioxopiperidin-3-yl]phenyl}piperidin-4-yl)methyl]-3-methylpiperazin-1-yl]phenyl}-3-(pyridin-4-yl)-1H-pyrazol-4-yl)-2-fluorophenyl]propane-1-sulfonamide (278)

LCMS: C₄₅H₅₀ClFN₈O₄S requires: 852.3, found: m/z=853.3 [M+H]⁺.

### N-[5-chloro-3-(1-{4-[(3S)-4-[(1-{5-[(3RS)-2,6-dioxopiperidin-3-yl]pyridin-2-yl}piperidin-4-yl)methyl]-3-methylpiperazin-1-yl]phenyl}-3-(pyridin-4-yl)-1H-pyrazol-4-yl)-2-fluorophenyl]propane-1-sulfonamide (279)

LCMS: C₄₄H₄₉ClFN₉O₄S requires: 853.3, found: m/z=854.3 [M+H]⁺.

### N-[5-chloro-3-(1-{4-[(3S)-4-({1-[4-(2,4-dioxo-1,3-diazinan-1-yl)phenyl]piperidin-4-yl}methyl)-3-methylpiperazin-1-yl]phenyl}-3-(pyridin-4-yl)-1H-pyrazol-4-yl)-2-fluorophenyl]propane-1-sulfonamide (280)

LCMS: C₄₄H₄₉ClFN₉O₄S requires: 853.3, found: m/z=854.3 [M+H]⁺.

### N-{5-chloro-3-[1-(4-{4-[(1-{4-[(3R)-2,6-dioxopiperidin-3-yl]phenyl}piperidin-4-yl)methyl]piperazin-1-yl}-2-fluorophenyl)-3-(pyridin-4-yl)-1H-pyrazol-4-yl]-2-fluorophenyl}pyrrolidine-1-sulfonamide (281)

¹H NMR (500 MHz, DMSO-d₆) δ 10.78 (s, 1H), 9.94 (s, 1H), 8.59-8.55 (m, 2H), 8.45 (s, 1H), 7.67 (s, 1H), 7.52 (dd, J=6.5, 2.7 Hz, 1H), 7.44-7.39 (m, 2H), 7.36 (dd, J=5.7, 2.7 Hz, 1H), 7.05 (d, J=8.2 Hz, 3H), 6.91 (d, J=8.3 Hz, 3H), 3.77-3.66 (m, 4H), 3.24 (s, 2H), 3.19-3.12 (m, 4H), 2.24 (s, 1H), 2.14 (d, J=11.1 Hz, 1H), 2.02 (dd, J=13.5, 5.2 Hz, 1H), 1.83 (d, J=12.7 Hz, 2H), 1.77-1.71 (m, 4H), 1.25 (s, 2H).

### N-{5-chloro-3-[1-(4-{4-[(1-{4-[(3R)-2,6-dioxopiperidin-3-yl]phenyl}piperidin-4-yl)methyl]piperazin-1-yl}phenyl)-3-(pyridin-4-yl)-1H-pyrazol-4-yl]-2-fluorophenyl}pyrrolidine-1-sulfonamide (282)

LCMS: C₄₅H₄₉ClFN₉O₄S requires: 865.3, found: m/z=866.2 [M+H]⁺.

### N-[5-chloro-3-(1-{4-[4-({1-[4-(2,4-dioxo-1,3-diazinan-1-yl)phenyl]piperidin-4-yl}methyl)piperazin-1-yl]phenyl}-3-(pyridin-4-yl)-1H-pyrazol-4-yl)-2-fluorophenyl]pyrrolidine-1-sulfonamide (283)

LCMS: C₄₄H₄₈ClFN₁₀O₄S requires: 866.3, found: m/z=867.3 [M+H]⁺.

### Rac-N-{5-chloro-3-[1-(4-{4-[(1-{4-[(3R)-2,6-dioxopiperidin-3-yl]phenyl}-4-fluoropiperidin-4-yl)methyl]piperazin-1-yl}phenyl)-3-(pyridin-4-yl)-1H-pyrazol-4-yl]-2-fluorophenyl}pyrrolidine-1-sulfonamide (284)

LCMS: C₄₅H₄₈ClF₂N₉O₄S requires: 883.3, found: m/z=884.3 [M+H]⁺.

### N-{5-chloro-3-[1-(4-{4-[(1-{5-[(3R)-2,6-dioxopiperidin-3-yl]pyridin-2-yl}piperidin-4-yl)methyl]piperazin-1-yl}phenyl)-3-(pyridin-4-yl)-1H-pyrazol-4-yl]-2-fluorophenyl}pyrrolidine-1-sulfonamide (285)

LCMS: C₄₄H₄₈ClFN₁₀O₄S requires: 866.3, found: m/z=867.4 [M+H]⁺.

### N-{5-chloro-3-[1-(5-{4-[(1-{4-[(3R)-2,6-dioxopiperidin-3-yl]phenyl}piperidin-4-yl)methyl]piperazin-1-yl}pyridin-2-yl)-3-(pyridin-4-yl)-1H-pyrazol-4-yl]-2-fluorophenyl}pyrrolidine-1-sulfonamide (286)

LCMS: C₄₄H₄₈ClFN₁₀O₄S requires: 866.3, found: m/z=867.2 [M+H]⁺.

### N-[5-chloro-3-(1-{5-[4-({1-[4-(2,4-dioxo-1,3-diazinan-1-yl)phenyl]piperidin-4-yl}methyl)piperazin-1-yl]pyridin-2-yl}-3-(pyridin-4-yl)-1H-pyrazol-4-yl)-2-fluorophenyl]pyrrolidine-1-sulfonamide (287)

LCMS: C₄₃H₄₇ClFN₁₁O₄S requires: 867.3, found: m/z=868.2 [M+H]⁺.

### Rac-N-{5-chloro-3-[1-(5-{4-[(1-{5-[(3R)-2,6-dioxopiperidin-3-yl]pyridin-2-yl}piperidin-4-yl)methyl]piperazin-1-yl}pyridin-2-yl)-3-(pyridin-4-yl)-1H-pyrazol-4-yl]-2-fluorophenyl}pyrrolidine-1-sulfonamide (288)

LCMS: C₄₃H₄₇ClFN₁₁O₄S requires: 867.3, found: m/z=868.3 [M+H]⁺.

### N-(3-(1-(4-((R)-4-((1-(5-((RS)-2,6-dioxopiperidin-3-yl)pyridin-2-yl)piperidin-4-yl)methyl)-3-methylpiperazin-1-yl)-2-fluorophenyl)-3-(pyridin-4-yl)-1H-pyrazol-4-yl)-2,5-difluorophenyl)pyrrolidine-1-sulfonamide (289)

LCMS: C₄₅H₄₉F₃N₁₀O₄S requires: 882.4, found: m/z=883.3 [M+H]⁺.

### N-{5-chloro-3-[1-(5-{4-[(1-{5-[(3R)-2,6-dioxopiperidin-3-yl]pyridin-2-yl}piperidin-4-yl)methyl]piperazin-1-yl}pyridin-2-yl)-3-(pyridin-4-yl)-1H-pyrazol-4-yl]-2-fluorophenyl}pyrrolidine-1-sulfonamide (290)

LCMS: C₄₃H₄₇ClFN₁₁O₄S requires: 867.3, found: m/z=868.3 [M+H]⁺.

### (3R)—N-{5-chloro-3-[1-(4-{4-[(1-{4-[(3R)-2,6-dioxopiperidin-3-yl]phenyl}piperidin-4-yl)methyl]piperazin-1-yl}phenyl)-3-(pyridin-4-yl)-1H-pyrazol-4-yl]-2-fluorophenyl}-3-fluoropyrrolidine-1-sulfonamide (291)

LCMS: C₄₅H₄₈ClF₂N₉O₄S requires: 883.3, found: m/z=884.2 [M+H]⁺.

### (3R)—N-[5-chloro-3-(1-{4-[4-({1-[4-(2,4-dioxo-1,3-diazinan-1-yl)phenyl]piperidin-4-yl}methyl)piperazin-1-yl]phenyl}-3-(pyridin-4-yl)-1H-pyrazol-4-yl)-2-fluorophenyl]-3-fluoropyrrolidine-1-sulfonamide (292)

LCMS: C₄₄H₄₇ClF₂N₁₀O₄S requires: 884.3, found: m/z=885.3 [M+H]⁺.

### (3R)—N-{5-chloro-3-[1-(4-{4-[(1-{5-[(3R)-2,6-dioxopiperidin-3-yl]pyridin-2-yl}piperidin-4-yl)methyl]piperazin-1-yl}phenyl)-3-(pyridin-4-yl)-1H-pyrazol-4-yl]-2-fluorophenyl}-3-fluoropyrrolidine-1-sulfonamide (293)

LCMS: C₄₄H₄₇ClF₂N₁₀O₄S requires: 884.3, found: m/z=885.4 [M+H]⁺.

### (3R)—N-{5-chloro-3-[1-(5-{4-[(1-{4-[(3R)-2,6-dioxopiperidin-3-yl]phenyl}piperidin-4-yl)methyl]piperazin-1-yl}pyridin-2-yl)-3-(pyridin-4-yl)-1H-pyrazol-4-yl]-2-fluorophenyl}-3-fluoropyrrolidine-1-sulfonamide (294)

LCMS: C₄₄H₄₇ClF₂N₁₀O₄S requires: 884.3, found: m/z=885.2 [M+H]⁺.

### (3R)—N-[5-chloro-3-(1-{5-[4-({1-[4-(2,4-dioxo-1,3-diazinan-1-yl)phenyl]piperidin-4-yl}methyl)piperazin-1-yl]pyridin-2-yl}-3-(pyridin-4-yl)-1H-pyrazol-4-yl)-2-fluorophenyl]-3-fluoropyrrolidine-1-sulfonamide (295)

LCMS: C₄₃H₄₆ClF₂N₁₁O₄S requires: 885.3, found: m/z=886.3 [M+H]⁺.

### (3R)—N-{5-chloro-3-[1-(5-{4-[(1-{5-[(3RS)-2,6-dioxopiperidin-3-yl]pyridin-2-yl}piperidin-4-yl)methyl]piperazin-1-yl}pyridin-2-yl)-3-(pyridin-4-yl)-1H-pyrazol-4-yl]-2-fluorophenyl}-3-fluoropyrrolidine-1-sulfonamide (296)

LCMS: C₄₃H₄₆ClF₂N₁₁O₄S requires: 885.3, found: m/z=886.2 [M+H]⁺.

### (3R)—N-{5-chloro-3-[1-(5-{4-[(1-{5-[(3R)-2,6-dioxopiperidin-3-yl]pyridin-2-yl}piperidin-4-yl)methyl]piperazin-1-yl}pyridin-2-yl)-3-(pyridin-4-yl)-1H-pyrazol-4-yl]-2-fluorophenyl}-3-fluoropyrrolidine-1-sulfonamide (297)

LCMS: C₄₃H₄₆ClF₂N₁₁O₄S requires: 885.3, found: m/z=886.4 [M+H]⁺. ¹H NMR (500 MHz, DMSO-d₆) δ 10.81 (s, 2H), 10.07 (s, 1H), 8.77 (s, 2H), 8.61-8.56 (m, 4H), 8.20 (d, J=2.9 Hz, 2H), 7.94 (d, J=2.5 Hz, 2H), 7.91 (d, J=9.0 Hz, 2H), 7.65 (dd, J=9.2, 2.9 Hz, 2H), 7.52 (dd, J=6.5, 2.7 Hz, 2H), 7.48-7.43 (m, 4H), 7.36 (td, J=7.8, 6.7, 2.7 Hz, 4H), 6.80 (d, J=8.9 Hz, 2H), 5.34 (s, 1H), 5.24 (t, J=3.5 Hz, 1H), 4.27 (d, J=12.7 Hz, 4H), 3.73 (dd, J=12.2, 4.9 Hz, 2H), 3.48-3.34 (m, 9H), 3.32-3.22 (m, 15H), 2.80 (t, J=12.2 Hz, 4H), 2.68 (ddd, J=17.1, 12.2, 5.2 Hz, 3H), 2.57 (s, 6H), 2.54 (d, J=4.1 Hz, 6H), 2.25 (d, J=6.6 Hz, 3H), 2.23-2.04 (m, 6H), 1.98 (ddt, J=13.6, 9.0, 4.1 Hz, 3H), 1.80 (d, J=13.2 Hz, 5H), 1.10 (dd, J=19.0, 7.4 Hz, 4H).

### (3R)—N-{5-chloro-3-[1-(5-{4-[(7-{4-[(3RS)-2,6-dioxopiperidin-3-yl]phenyl}-7-azaspiro[3.5]nonan-2-yl)methyl]piperazin-1-yl}pyridin-2-yl)-3-(pyridin-4-yl)-1H-pyrazol-4-yl]-2-fluorophenyl}-3-fluoropyrrolidine-1-sulfonamide (298)

LCMS: C₄₇H₅₁ClF₂N₁₀O₄S requires: 924.3, found: m/z=925.2 [M+H]⁺.

### N-{3-[1-(4-{4-[(1-{4-[(3R)-2,6-dioxopiperidin-3-yl]phenyl}piperidin-4-yl)methyl]piperazin-1-yl}phenyl)-3-(pyridin-4-yl)-1H-pyrazol-4-yl]-2,5-difluorophenyl}pyrrolidine-1-sulfonamide (299)

LCMS: C₄₅H₄₉F₂N₉O₄S requires: 849.4, found: m/z=850.2 [M+H]⁺.

### N-[3-(1-{4-[4-({1-[4-(2,4-dioxo-1,3-diazinan-1-yl)phenyl]piperidin-4-yl}methyl)piperazin-1-yl]phenyl}-3-(pyridin-4-yl)-1H-pyrazol-4-yl)-2,5-difluorophenyl]pyrrolidine-1-sulfonamide (300)

LCMS: C₄₄H₄₈F₂N₁₀O₄S requires: 850.4, found: m/z=851.3 [M+H]⁺.

### N-{3-[1-(4-{4-[(1-{5-[(3R)-2,6-dioxopiperidin-3-yl]pyridin-2-yl}piperidin-4-yl)methyl]piperazin-1-yl}phenyl)-3-(pyridin-4-yl)-1H-pyrazol-4-yl]-2,5-difluorophenyl}pyrrolidine-1-sulfonamide (301)

LCMS: C₄₄H₄₈F₂N₁₀O₄S requires: 850.4, found: m/z=851.4 [M+H]⁺.

### (3R)—N-{3-[1-(5-{4-[(1-{4-[(3R)-2,6-dioxopiperidin-3-yl]phenyl}piperidin-4-yl)methyl]piperazin-1-yl}pyridin-2-yl)-3-(pyridin-4-yl)-1H-pyrazol-4-yl]-2,5-difluorophenyl}-3-fluoropyrrolidine-1-sulfonamide (302)

LCMS: C₄₄H₄₇F₃N₁₀O₄S requires: 868.3, found: m/z=869.3 [M+H]⁺.

### (3R)—N-[3-(1-{5-[4-({1-[4-(2,4-dioxo-1,3-diazinan-1-yl)phenyl]piperidin-4-yl}methyl)piperazin-1-yl]pyridin-2-yl}-3-(pyridin-4-yl)-1H-pyrazol-4-yl)-2,5-difluorophenyl]-3-fluoropyrrolidine-1-sulfonamide (303)

LCMS: C₄₃H₄₆F₃N₁₁O₄S requires: 869.3, found: m/z=870.3 [M+H]⁺.

### (3R)—N-{3-[1-(5-{4-[(1-{5-[(3RS)-2,6-dioxopiperidin-3-yl]pyridin-2-yl}piperidin-4-yl)methyl]piperazin-1-yl}pyridin-2-yl)-3-(pyridin-4-yl)-1H-pyrazol-4-yl]-2,5-difluorophenyl}-3-fluoropyrrolidine-1-sulfonamide (304)

LCMS: C₄₃H₄₆F₃N₁₁O₄S requires: 869.3, found: m/z=870.3 [M+H]⁺.

### (3R)—N-{3-[1-(5-{4-[(1-{5-[(3R)-2,6-dioxopiperidin-3-yl]pyridin-2-yl}piperidin-4-yl)methyl]piperazin-1-yl}pyridin-2-yl)-3-(pyridin-4-yl)-1H-pyrazol-4-yl]-2,5-difluorophenyl}-3-fluoropyrrolidine-1-sulfonamide (305)

LCMS: C₄₃H₄₆F₃N₁₁O₄S requires: 869.3, found: m/z=870.4 [M+H]⁺. ¹H NMR (500 MHz, DMSO-d₆) δ 10.81 (s, 2H), 10.08 (s, 1H), 8.75 (s, 2H), 8.61-8.56 (m, 4H), 8.20 (d, J=2.9 Hz, 2H), 7.94 (d, J=2.5 Hz, 2H), 7.91 (d, J=9.0 Hz, 2H), 7.65 (dd, J=9.1, 2.9 Hz, 2H), 7.48-7.43 (m, 4H), 7.40-7.28 (m, 4H), 7.14 (d, J=10.1 Hz, 2H), 6.80 (d, J=8.9 Hz, 2H), 5.34 (s, 1H), 5.24 (t, J=3.6 Hz, 1H), 4.27 (d, J=12.7 Hz, 4H), 3.73 (dd, J=12.1, 4.9 Hz, 2H), 3.48-3.34 (m, 8H), 3.32-3.23 (m, 13H), 2.79 (t, J=12.3 Hz, 4H), 2.68 (ddd, J=17.6, 12.4, 5.4 Hz, 3H), 2.55 (q, J=7.7, 6.2 Hz, 11H), 2.24 (d, J=6.7 Hz, 3H), 2.22-2.03 (m, 6H), 1.97 (dt, J=13.5, 4.5 Hz, 3H), 1.80 (d, J=13.2 Hz, 5H), 1.10 (td, J=10.8, 5.8 Hz, 4H).

### (3R)-3-(4-{4-[(4-{4-[4-(5-chloro-3-{[ethyl(methyl)sulfamoyl]amino}-2-fluorophenyl)-3-(pyridin-4-yl)-1H-pyrazol-1-yl]phenyl}piperazin-1-yl)methyl]piperidin-1-yl}phenyl)piperidine-2,6-dione (306)

LCMS: C₄₄H₄₉ClFN₉O₄S requires: 853.3, found: m/z=854.3 [M+H]⁺.

### 1-(4-{4-[(4-{4-[4-(5-chloro-3-{[ethyl(methyl)sulfamoyl]amino}-2-fluorophenyl)-3-(pyridin-4-yl)-1H-pyrazol-1-yl]phenyl}piperazin-1-yl)methyl]piperidin-1-yl}phenyl)-1,3-diazinane-2,4-dione (307)

LCMS: C₄₃H₄₈ClFN₁₀O₄S requires: 854.3, found: m/z=855.2 [M+H]⁺.

### Rac-(3R)-3-(6-{4-[(4-{4-[4-(5-chloro-3-{[ethyl(methyl)sulfamoyl]amino}-2-fluorophenyl)-3-(pyridin-4-yl)-1H-pyrazol-1-yl]phenyl}piperazin-1-yl)methyl]piperidin-1-yl}pyridin-3-yl)piperidine-2,6-dione (308)

LCMS: C₄₃H₄₈ClFN₁₀O₄S requires: 854.3, found: m/z=855.3 [M+H]⁺.

### (3R)-3-(6-{4-[(4-{4-[4-(5-chloro-3-{[ethyl(methyl)sulfamoyl]amino}-2-fluorophenyl)-3-(pyridin-4-yl)-1H-pyrazol-1-yl]phenyl}piperazin-1-yl)methyl]piperidin-1-yl}pyridin-3-yl)piperidine-2,6-dione (309)

LCMS: C₄₃H₄₈ClFN₁₀O₄S requires: 854.3, found: m/z=855.4 [M+H]⁺. ¹H NMR (500 MHz, DMSO-d₆) δ 10.81 (s, 2H), 9.94 (s, 1H), 8.75 (s, 2H), 8.59-8.54 (m, 4H), 7.95 (d, J=2.6 Hz, 2H), 7.79 (d, J=9.0 Hz, 4H), 7.49-7.34 (m, 11H), 7.11 (d, J=8.8 Hz, 4H), 6.80 (d, J=8.9 Hz, 2H), 4.28 (d, J=12.8 Hz, 4H), 3.73 (dd, J=12.1, 4.9 Hz, 3H), 3.24 (s, 4H), 3.09 (q, J=7.1 Hz, 5H), 2.80 (t, J=12.3 Hz, 5H), 2.68 (s, 8H), 2.53 (s, 9H), 2.25 (s, 1H), 2.17 (td, J=12.5, 4.3 Hz, 3H), 2.00 (s, 1H), 1.80 (d, J=13.1 Hz, 5H), 1.13 (s, 2H), 1.12-1.02 (m, 8H), 1.01 (s, 2H).

### (3R)-3-(4-{4-[(4-{6-[4-(5-chloro-3-{[ethyl(methyl)sulfamoyl]amino}-2-fluorophenyl)-3-(pyridin-4-yl)-1H-pyrazol-1-yl]pyridin-3-yl}piperazin-1-yl)methyl]piperidin-1-yl}phenyl)piperidine-2,6-dione (310)

LCMS: C₄₃H₄₈ClFN₁₀O₄S requires: 854.3, found: m/z=855.2 [M+H]⁺.

### 1-(4-{4-[(4-{6-[4-(5-chloro-3-{[ethyl(methyl)sulfamoyl]amino}-2-fluorophenyl)-3-(pyridin-4-yl)-1H-pyrazol-1-yl]pyridin-3-yl}piperazin-1-yl)methyl]piperidin-1-yl}phenyl)-1,3-diazinane-2,4-dione (311)

LCMS: C₄₂H₄₇ClFN₁₁O₄S requires: 855.3, found: m/z=856.3 [M+H]⁺.

### Rac-(3R)-3-(6-{4-[(4-{6-[4-(5-chloro-3-{[ethyl(methyl)sulfamoyl]amino}-2-fluorophenyl)-3-(pyridin-4-yl)-1H-pyrazol-1-yl]pyridin-3-yl}piperazin-1-yl)methyl]piperidin-1-yl}pyridin-3-yl)piperidine-2,6-dione (312)

LCMS: C₄₂H₄₇ClFN₁₁O₄S requires: 855.3, found: m/z=856.2 [M+H]⁺.

### 1-(6-{4-[(4-{6-[4-(5-chloro-3-{[ethyl(methyl)sulfamoyl]amino}-2-fluorophenyl)-3-(pyridin-4-yl)-1H-pyrazol-1-yl]pyridin-3-yl}piperazin-1-yl)methyl]piperidin-1-yl}pyridin-3-yl)-1,3-diazinane-2,4-dione (313)

LCMS: C₄₁H₄₆ClFN₁₂O₄S requires: 856.3, found: m/z=857.4 [M+H]⁺.

### (3R)-3-(6-{4-[(4-{6-[4-(5-chloro-3-{[ethyl(methyl)sulfamoyl]amino}-2-fluorophenyl)-3-(pyridin-4-yl)-1H-pyrazol-1-yl]pyridin-3-yl}piperazin-1-yl)methyl]piperidin-1-yl}pyridin-3-yl)piperidine-2,6-dione (314)

LCMS: C₄₂H₄₇ClFN₁₁O₄S requires: 855.3, found: m/z=856.4 [M+H]⁺. ¹H NMR (500 MHz, DMSO-d₆) δ 10.81 (s, 1H), 8.79 (s, 1H), 8.61-8.56 (m, 2H), 8.20 (d, J=2.9 Hz, 1H), 7.94 (d, J=2.6 Hz, 1H), 7.91 (d, J=9.0 Hz, 1H), 7.64 (dd, J=9.2, 2.8 Hz, 1H), 7.49-7.43 (m, 3H), 7.37 (dq, J=5.7, 2.5 Hz, 2H), 6.80 (d, J=8.9 Hz, 1H), 4.27 (d, J=12.7 Hz, 2H), 3.73 (dd, J=12.1, 4.9 Hz, 1H), 3.28 (d, J=5.9 Hz, 4H), 3.09 (q, J=7.1 Hz, 2H), 2.79 (t, J=11.8 Hz, 2H), 2.68 (s, 4H), 2.55 (d, J=12.7 Hz, 4H), 2.24 (d, J=6.7 Hz, 2H), 2.17 (td, J=12.5, 4.3 Hz, 1H), 1.98 (dq, J=8.3, 4.4 Hz, 1H), 1.80 (d, J=13.1 Hz, 2H), 1.10 (td, J=10.7, 5.7 Hz, 2H), 1.02 (t, J=7.1 Hz, 3H).

### (3R)—N-[5-chloro-3-(1-{4-[(3R)-4-({1-[4-(2,4-dioxo-1,3-diazinan-1-yl)phenyl]piperidin-4-yl}methyl)-3-methylpiperazin-1-yl]phenyl}-3-(pyridin-4-yl)-1H-pyrazol-4-yl)-2-fluorophenyl]-3-fluoropyrrolidine-1-sulfonamide (315)

LCMS: C₄₅H₄₉ClF₂N₁₀O₄S requires: 898.3, found: m/z=899.2 [M+H]⁺.

### N-[5-chloro-3-(1-{4-[(3R)-4-[(1-{4-[(3R)-2,6-dioxopiperidin-3-yl]phenyl}piperidin-4-yl)methyl]-3-methylpiperazin-1-yl]phenyl}-3-(pyridin-4-yl)-1H-pyrazol-4-yl)-2-fluorophenyl]pyrrolidine-1-sulfonamide (316)

LCMS: C₄₆H₅₁ClFN₉O₄S requires: 879.3, found: m/z=880.3 [M+H]⁺.

### (3R)—N-[5-chloro-3-(1-{4-[(3R)-4-[(1-{4-[(3R)-2,6-dioxopiperidin-3-yl]phenyl}piperidin-4-yl)methyl]-3-methylpiperazin-1-yl]phenyl}-3-(pyridin-4-yl)-1H-pyrazol-4-yl)-2-fluorophenyl]-3-fluoropyrrolidine-1-sulfonamide (317)

LCMS: C₄₆H₅₀ClF₂N₉O₄S requires: 897.3, found: m/z=898.2 [M+H]⁺.

### (3R)—N-[5-chloro-3-(1-{4-[(3R)-4-[(1-{5-[(3RS)-2,6-dioxopiperidin-3-yl]pyridin-2-yl}piperidin-4-yl)methyl]-3-methylpiperazin-1-yl]phenyl}-3-(pyridin-4-yl)-1H-pyrazol-4-yl)-2-fluorophenyl]-3-fluoropyrrolidine-1-sulfonamide (318)

LCMS: C₄₅H₄₉ClF₂N₁₀O₄S requires: 898.3, found: m/z=899.2 [M+H]⁺.

### N-[5-chloro-3-(1-{4-[(3R)-4-({1-[4-(2,4-dioxo-1,3-diazinan-1-yl)phenyl]piperidin-4-yl}methyl)-3-methylpiperazin-1-yl]phenyl}-3-(pyridin-4-yl)-1H-pyrazol-4-yl)-2-fluorophenyl]pyrrolidine-1-sulfonamide (319)

LCMS: C₄₅H₅₀ClFN₁₀O₄S requires: 880.3, found: m/z=881.3 [M+H]⁺.

### (3R)—N-[5-chloro-3-(1-{5-[(3R)-4-[(1-{4-[(3R)-2,6-dioxopiperidin-3-yl]phenyl}piperidin-4-yl)methyl]-3-methylpiperazin-1-yl]pyridin-2-yl}-3-(pyridin-4-yl)-1H-pyrazol-4-yl)-2-fluorophenyl]-3-fluoropyrrolidine-1-sulfonamide (320)

¹H NMR (500 MHz, DMSO-d₆) δ 10.77 (s, 1H), 10.08 (s, 1H), 8.77 (s, 1H), 8.61-8.56 (m, 2H), 8.21 (s, 1H), 7.91 (d, J=8.9 Hz, 1H), 7.65 (s, 1H), 7.52 (dd, J=6.4, 2.6 Hz, 1H), 7.48-7.43 (m, 2H), 7.37 (s, 1H), 7.04 (d, J=8.5 Hz, 2H), 6.90 (d, J=8.4 Hz, 2H), 5.34 (s, 1H), 5.24 (s, 1H), 3.72 (dd, J=11.1, 5.0 Hz, 1H), 3.68 (s, 2H), 3.58 (s, 2H), 3.46 (s, 1H), 3.45-3.37 (m, 1H), 3.40-3.35 (m, 1H), 3.30-3.23 (m, 1H), 2.99 (s, 2H), 2.68 (s, 3H), 2.16-2.10 (m, 1H), 2.05-1.99 (m, 1H), 1.91 (d, J=13.7 Hz, 1H), 1.73 (s, 1H), 1.24 (s, 3H), 1.10 (s, 3H).

### (3R)—N-[5-chloro-3-(1-{5-[(3R)-4-[(1-{5-[(3RS)-2,6-dioxopiperidin-3-yl]pyridin-2-yl}piperidin-4-yl)methyl]-3-methylpiperazin-1-yl]pyridin-2-yl}-3-(pyridin-4-yl)-1H-pyrazol-4-yl)-2-fluorophenyl]-3-fluoropyrrolidine-1-sulfonamide (321)

LCMS: C₄₄H₄₈ClF₂N₁₁O₄S requires: 899.3, found: m/z=900.2 [M+H]⁺.

### (3R)—N-[5-chloro-3-(1-{5-[(3R)-4-({1-[4-(2,4-dioxo-1,3-diazinan-1-yl)phenyl]piperidin-4-yl}methyl)-3-methylpiperazin-1-yl]pyridin-2-yl}-3-(pyridin-4-yl)-1H-pyrazol-4-yl)-2-fluorophenyl]-3-fluoropyrrolidine-1-sulfonamide (322)

LCMS: C₄₄H₄₈ClF₂N₁₁O₄S requires: 899.3, found: m/z=900.3 [M+H]⁺.

### N-[5-chloro-3-(1-{5-[(3R)-4-[(1-{5-[(3RS)-2,6-dioxopiperidin-3-yl]pyridin-2-yl}piperidin-4-yl)methyl]-3-methylpiperazin-1-yl]pyridin-2-yl}-3-(pyridin-4-yl)-1H-pyrazol-4-yl)-2-fluorophenyl]pyrrolidine-1-sulfonamide (323)

LCMS: C₄₄H₄₉ClFN₁₁O₄S requires: 881.3, found: m/z=882.4 [M+H]⁺. ¹H NMR (500 MHz, DMSO-d₆) δ 10.82 (s, 1H), 9.94 (s, 1H), 8.81 (s, 1H), 8.59 (d, J=5.3 Hz, 2H), 8.31 (s, 1H), 7.96 (s, 2H), 7.75 (s, 1H), 7.52 (dd, J=6.5, 2.7 Hz, 1H), 7.48-7.43 (m, 2H), 7.40 (dd, J=5.7, 2.7 Hz, 1H), 6.84 (s, 1H), 4.33 (s, 2H), 4.02 (s, 1H), 3.74 (dd, J=12.0, 4.9 Hz, 1H), 3.58 (s, OH), 3.48 (s, 1H), 3.20 (s, 2H), 3.18-3.12 (m, 2H), 3.00 (s, 2H), 2.84 (s, 2H), 2.18 (dd, J=14.0, 9.9 Hz, 1H), 2.01-1.94 (m, 1H), 1.91 (s, 1H), 1.77-1.71 (m, 3H), 1.39 (s, 2H), 1.25 (s, 3H), 1.08 (s, 1H).

### N-[5-chloro-3-(1-{5-[(3R)-4-({1-[4-(2,4-dioxo-1,3-diazinan-1-yl)phenyl]piperidin-4-yl}methyl)-3-methylpiperazin-1-yl]pyridin-2-yl}-3-(pyridin-4-yl)-1H-pyrazol-4-yl)-2-fluorophenyl]pyrrolidine-1-sulfonamide (324)

LCMS: C₄₄H₄₉ClFN₁₁O₄S requires: 881.3, found: m/z=882.4 [M+H]⁺.

### N-[5-chloro-3-(1-{5-[(3R)-4-[(1-{5-[(3R)-2,6-dioxopiperidin-3-yl]pyridin-2-yl}piperidin-4-yl)methyl]-3-methylpiperazin-1-yl]pyridin-2-yl}-3-(pyridin-4-yl)-1H-pyrazol-4-yl)-2-fluorophenyl]pyrrolidine-1-sulfonamide (325)

LCMS: C₄₄H₄₉ClFN₁₁O₄S requires: 881.3, found: m/z=882.3 [M+H]⁺.

### N-[5-chloro-3-(1-{5-[(3R)-4-[(1-{4-[(3R)-2,6-dioxopiperidin-3-yl]phenyl}piperidin-4-yl)methyl]-3-methylpiperazin-1-yl]pyridin-2-yl}-3-(pyridin-4-yl)-1H-pyrazol-4-yl)-2-fluorophenyl]pyrrolidine-1-sulfonamide (326)

LCMS: C₄₅H₅₀ClFN₁₀O₄S requires: 880.3, found: m/z=881.3 [M+H]⁺. ¹H NMR (500 MHz, DMSO-d₆) δ 10.77 (s, 1H), 9.94 (s, 1H), 8.78 (s, 1H), 8.61-8.56 (m, 2H), 8.20 (s, 1H), 7.91 (d, J=9.0 Hz, 1H), 7.65 (d, J=9.2 Hz, 1H), 7.52 (dd, J=6.5, 2.7 Hz, 1H), 7.47-7.42 (m, 2H), 7.41-7.37 (m, 1H), 7.04 (d, J=8.6 Hz, 2H), 6.90 (d, J=8.5 Hz, 2H), 3.76-3.65 (m, 3H), 3.58 (s, 2H), 3.34 (dd, J=10.8, 6.2 Hz, 5H), 3.18-3.12 (m, 2H), 2.98 (s, 2H), 2.69 (d, J=13.1 Hz, 2H), 2.64-2.58 (m, OH), 2.31 (s, 1H), 2.08 (s, 1H), 2.02 (s, 2H), 1.91 (d, J=12.9 Hz, 1H), 1.77-1.70 (m, 4H), 1.26 (d, J=13.7 Hz, 1H), 1.20 (s, 1H), 1.09 (s, 2H).

### (RS)—N-(5-chloro-3-(1-(4-(4-((1-(5-((R)-2,6-dioxopiperidin-3-yl)pyridin-2-yl)piperidin-4-yl)methyl)piperazin-1-yl)phenyl)-3-(pyridin-4-yl)-1H-pyrazol-4-yl)-2-fluorophenyl)butane-2-sulfonamide (327)

LCMS: C₄₄H₄₉ClFN₉O₄S requires: 853.3, found: m/z=854.4 [M+H]⁺.

### Rac-(2R)—N-[5-chloro-3-(1-{4-[4-({1-[4-(2,4-dioxo-1,3-diazinan-1-yl)phenyl]piperidin-4-yl}methyl)piperazin-1-yl]phenyl}-3-(pyridin-4-yl)-1H-pyrazol-4-yl)-2-fluorophenyl]butane-2-sulfonamide (328)

LCMS: C₄₄H₄₉ClFN₉O₄S requires: 853.3, found: m/z=854.3 [M+H]⁺.

### Rac-(R)—N-(5-chloro-3-(1-(4-(4-((1-(5-((S)-2,6-dioxopiperidin-3-yl)pyridin-2-yl)piperidin-4-yl)methyl)piperazin-1-yl)phenyl)-3-(pyridin-4-yl)-1H-pyrazol-4-yl)-2-fluorophenyl)butane-2-sulfonamide (329)

LCMS: C₄₄H₄₉ClFN₉O₄S requires: 853.3, found: m/z=854.3 [M+H]⁺.

### (2RS)—N-{5-chloro-3-[1-(4-{4-[(1-{4-[(3R)-2,6-dioxopiperidin-3-yl]phenyl}piperidin-4-yl)methyl]piperazin-1-yl}phenyl)-3-(pyridin-4-yl)-1H-pyrazol-4-yl]-2-fluorophenyl}butane-2-sulfonamide (330)

LCMS: C₄₅H₅₀ClFN₈O₄S requires: 852.3, found: m/z=853.4 [M+H]⁺.

### N-{5-chloro-3-[1-(4-{4-[(1-{5-[(3R)-2,6-dioxopiperidin-3-yl]pyridin-2-yl}piperidin-4-yl)methyl]piperazin-1-yl}phenyl)-3-(pyridin-4-yl)-1H-pyrazol-4-yl]-2-fluorophenyl}-3-fluoroazetidine-1-sulfonamide (331)

LCMS: C₄₃H₄₅ClF₂N₁₀O₄S requires: 870.3, found: m/z=871.2 [M+H]⁺.

### Rac-N-{5-chloro-3-[1-(4-{4-[(1-{5-[(3R)-2,6-dioxopiperidin-3-yl]pyridin-2-yl}piperidin-4-yl)methyl]piperazin-1-yl}phenyl)-3-(pyridin-4-yl)-1H-pyrazol-4-yl]-2-fluorophenyl}-3-fluoroazetidine-1-sulfonamide (332)

LCMS: C₄₃H₄₅ClF₂N₁₀O₄S requires: 870.3, found: m/z=871.2 [M+H]⁺.

### N-{5-chloro-3-[1-(4-{4-[(1-{4-[(3R)-2,6-dioxopiperidin-3-yl]phenyl}piperidin-4-yl)methyl]piperazin-1-yl}phenyl)-3-(pyridin-4-yl)-1H-pyrazol-4-yl]-2-fluorophenyl}-3-fluoroazetidine-1-sulfonamide (333)

LCMS: C₄₄H₄₆ClF₂N₉O₄S requires: 869.3, found: m/z=870.2 [M+H]⁺.

### (3R)—N-{5-chloro-3-[1-(4-{4-[(1-{5-[(3R)-2,6-dioxopiperidin-3-yl]pyridin-2-yl}piperidin-4-yl)methyl]piperazin-1-yl}-2-fluorophenyl)-3-(pyridin-4-yl)-1H-pyrazol-4-yl]-2-fluorophenyl}-3-fluoropyrrolidine-1-sulfonamide (334)

LCMS: C₄₄H₄₆ClF₃N₁₀O₄S requires: 902.3, found: m/z=903.2 [M+H]⁺.

### (3R)—N-{5-chloro-3-[1-(4-{4-[(1-{5-[(3RS)-2,6-dioxopiperidin-3-yl]pyridin-2-yl}piperidin-4-yl)methyl]piperazin-1-yl}-2-fluorophenyl)-3-(pyridin-4-yl)-1H-pyrazol-4-yl]-2-fluorophenyl}-3-fluoropyrrolidine-1-sulfonamide (335)

LCMS: C₄₄H₄₆ClF₃N₁₀O₄S requires: 902.3, found: m/z=903.2 [M+H]⁺.

### (3R)—N-{5-chloro-3-[1-(4-{4-[(1-{4-[(3R)-2,6-dioxopiperidin-3-yl]phenyl}piperidin-4-yl)methyl]piperazin-1-yl}-2-fluorophenyl)-3-(pyridin-4-yl)-1H-pyrazol-4-yl]-2-fluorophenyl}-3-fluoropyrrolidine-1-sulfonamide (336)

LCMS: C₄₅H₄₇ClF₃N₉O₄S requires: 901.3, found: m/z=902.3 [M+H]⁺.

### Rac-N-{5-chloro-3-[1-(4-{4-[(1-{5-[(3R)-2,6-dioxopiperidin-3-yl]pyridin-2-yl}piperidin-4-yl)methyl]piperazin-1-yl}-2-fluorophenyl)-3-(pyridin-4-yl)-1H-pyrazol-4-yl]-2-fluorophenyl}-3-fluoroazetidine-1-sulfonamide (337)

LCMS: C₄₃H₄₄ClF₃N₁₀O₄S requires: 888.3, found: m/z=889.2 [M+H]⁺. ¹H NMR (500 MHz, DMSO-d₆) δ 12.74 (s, 1H), 10.81 (s, 1H), 8.60-8.55 (m, 2H), 8.46 (d, J=2.0 Hz, 1H), 8.14 (s, 1H), 7.95 (d, J=2.5 Hz, 1H), 7.68 (t, J=9.0 Hz, 1H), 7.50 (dd, J=6.4, 2.7 Hz, 1H), 7.45-7.40 (m, 2H), 7.40-7.35 (m, 2H), 7.07 (d, J=14.9 Hz, 1H), 6.95 (d, J=9.3 Hz, 1H), 6.81 (d, J=8.8 Hz, 1H), 5.35 (dd, J=6.9, 3.1 Hz, OH), 5.23 (td, J=6.1, 3.1 Hz, OH), 4.28 (d, J=12.8 Hz, 2H), 4.06 (ddd, J=19.7, 10.4, 6.1 Hz, 2H), 3.89 (ddd, J=24.5, 10.7, 3.8 Hz, 2H), 3.73 (dd, J=12.1, 4.9 Hz, 1H), 2.81 (t, J=12.4 Hz, 2H), 2.74-2.65 (m, 1H), 2.24-2.13 (m, 1H), 2.01-1.94 (m, 1H), 1.88 (s, 1H), 1.80 (d, J=12.9 Hz, 2H), 1.13 (d, J=12.2 Hz, 2H).

### N-{5-chloro-3-[1-(4-{4-[(1-{4-[(3R)-2,6-dioxopiperidin-3-yl]phenyl}piperidin-4-yl)methyl]piperazin-1-yl}-2-fluorophenyl)-3-(pyridin-4-yl)-1H-pyrazol-4-yl]-2-fluorophenyl}-3-fluoroazetidine-1-sulfonamide (338)

LCMS: C₄₄H₄₅ClF₃N₉O₄S requires: 887.3, found: m/z=888.2 [M+H]⁺. ¹H NMR (500 MHz, DMSO-d₆) δ 10.77 (s, 1H), 10.23 (s, 1H), 8.60-8.55 (m, 2H), 8.45 (d, J=2.0 Hz, 1H), 7.67 (t, J=9.1 Hz, 1H), 7.50 (dd, J=6.4, 2.7 Hz, 1H), 7.45-7.40 (m, 2H), 7.35 (s, 1H), 7.09-7.02 (m, 3H), 6.95 (d, J=9.1 Hz, 1H), 6.90 (d, J=8.8 Hz, 2H), 5.34 (td, J=6.1, 3.1 Hz, OH), 5.23 (ddd, J=9.9, 6.0, 3.9 Hz, 1H), 4.06 (ddd, J=19.5, 10.0, 6.0 Hz, 2H), 3.88 (ddd, J=24.5, 10.5, 3.8 Hz, 2H), 3.76-3.65 (m, 3H), 3.35 (s, 2H), 2.68 (d, J=12.2 Hz, 2H), 2.64-2.58 (m, 1H), 2.50-2.42 (m, 1H), 2.33 (s, 2H), 2.19-2.06 (m, 1H), 2.05-1.98 (m, 1H), 1.82 (d, J=12.7 Hz, 2H), 1.25 (q, J=11.4 Hz, 2H).

### N-[5-chloro-3-(1-{4-[4-({1-[4-(2,4-dioxo-1,3-diazinan-1-yl)phenyl]piperidin-4-yl}methyl)piperazin-1-yl]-2-fluorophenyl}-3-(pyridin-4-yl)-1H-pyrazol-4-yl)-2-fluorophenyl]-3-fluoroazetidine-1-sulfonamide (339)

LCMS: C₄₃H₄₄ClF₃N₁₀O₄S requires: 888.3, found: m/z=889.1 [M+H]⁺. ¹H NMR (500 MHz, DMSO-d₆) δ 10.26 (s, 1H), 8.60-8.55 (m, 2H), 8.45 (d, J=2.0 Hz, 1H), 7.67 (t, J=9.1 Hz, 1H), 7.50 (dd, J=6.4, 2.7 Hz, 1H), 7.45-7.40 (m, 2H), 7.35 (s, 1H), 7.17-7.12 (m, 2H), 7.05 (d, J=14.9 Hz, 1H), 6.97-6.91 (m, 2H), 5.38-5.31 (m, OH), 5.22 (dd, J=7.0, 3.1 Hz, 1H), 4.11-4.00 (m, 2H), 3.88 (ddd, J=24.7, 10.4, 3.8 Hz, 2H), 3.73-3.67 (m, 4H), 3.35 (s, 1H), 2.69 (h, J=5.0 Hz, 4H), 2.59 (s, 4H), 2.32 (s, 3H), 1.83 (d, J=12.9 Hz, 2H), 1.76 (s, 1H), 1.30-1.22 (m, 2H).

### N-{3-[1-(4-{4-[(1-{4-[(3R)-2,6-dioxopiperidin-3-yl]phenyl}piperidin-4-yl)methyl]piperazin-1-yl}-2-fluorophenyl)-3-(pyridin-4-yl)-1H-pyrazol-4-yl]-2,5-difluorophenyl}pyrrolidine-1-sulfonamide (340)

LCMS: C₄₅H₄₈F₃N₉O₄S requires: 867.4, found: m/z=868.2 [M+H]⁺.

### Rac-N-{3-[1-(4-{4-[(1-{5-[(3R)-2,6-dioxopiperidin-3-yl]pyridin-2-yl}piperidin-4-yl)methyl]piperazin-1-yl}-2-fluorophenyl)-3-(pyridin-4-yl)-1H-pyrazol-4-yl]-2,5-difluorophenyl}pyrrolidine-1-sulfonamide (341)

LCMS: C₄₄H₄₇F₃N₁₀O₄S requires: 868.3, found: m/z=869.2 [M+H]⁺.

### N-{3-[1-(5-{4-[(1-{4-[(3R)-2,6-dioxopiperidin-3-yl]phenyl}piperidin-4-yl)methyl]piperazin-1-yl}pyrimidin-2-yl)-3-(pyridin-4-yl)-1H-pyrazol-4-yl]-2,5-difluorophenyl}pyrrolidine-1-sulfonamide (342)

LCMS: C₄₃H₄₇F₂N₁₁O₄S requires: 851.4, found: m/z=852.3 [M+H]⁺.

### Rac-N-{3-[1-(5-{4-[(1-{5-[(3R)-2,6-dioxopiperidin-3-yl]pyridin-2-yl}piperidin-4-yl)methyl]piperazin-1-yl}pyrimidin-2-yl)-3-(pyridin-4-yl)-1H-pyrazol-4-yl]-2,5-difluorophenyl}pyrrolidine-1-sulfonamide (343)

LCMS: C₄₂H₄₆F₂N₁₂O₄S requires: 852.3, found: m/z=853.2 [M+H]⁺.

### N-{5-chloro-3-[1-(4-{4-[(1-{4-[(3R)-2,6-dioxopiperidin-3-yl]-3-fluorophenyl}piperidin-4-yl)methyl]piperazin-1-yl}phenyl)-3-(pyridin-4-yl)-1H-pyrazol-4-yl]-2-fluorophenyl}pyrrolidine-1-sulfonamide (344)

¹H NMR (500 MHz, DMSO-d₆) δ 10.73 (s, 1H), 9.86 (s, 1H), 8.65 (s, 1H), 8.51-8.46 (m, 2H), 7.73-7.68 (m, 2H), 7.46-7.41 (m, 1H), 7.38-7.33 (m, 2H), 7.04 (s, 1H), 7.03-6.96 (m, 2H), 6.64 (d, J=10.0 Hz, 2H), 3.81 (dd, J=12.4, 5.0 Hz, 1H), 3.65 (d, J=12.3 Hz, 2H), 3.15 (s, 5H), 3.06 (s, 5H), 2.63 (t, J=12.1 Hz, 3H), 2.16 (d, J=7.0 Hz, 2H), 2.07 (dd, J=13.0, 4.2 Hz, 1H), 1.91-1.85 (m, 1H), 1.73 (d, J=13.6 Hz, 2H), 1.69-1.63 (m, 4H), 1.18-1.10 (m, 3H).

### Rac-N-{5-chloro-3-[1-(4-{4-[(1-{5-[(3R)-2,6-dioxopiperidin-3-yl]pyridin-2-yl}piperidin-4-yl)methyl]-1,4-diazepan-1-yl}-2-fluorophenyl)-3-(pyridin-4-yl)-1H-pyrazol-4-yl]-2-fluorophenyl}pyrrolidine-1-sulfonamide (345)

LCMS: C₄₅H₄₉ClF₂N₁₀O₄S requires: 898.3, found: m/z=899.2 [M+H]⁺. ¹H NMR (500 MHz, DMSO-d₆) δ 10.81 (s, 1H), 9.93 (s, 1H), 8.59-8.54 (m, 2H), 8.40 (d, J=1.9 Hz, 1H), 8.14 (s, 1H), 7.94 (s, 1H), 7.63 (s, 1H), 7.51 (dd, J=6.4, 2.7 Hz, 1H), 7.43-7.38 (m, 2H), 7.35 (dd, J=5.7, 2.7 Hz, 1H), 6.82 (s, 3H), 6.74 (s, 1H), 4.29 (s, 2H), 3.73 (d, J=10.1 Hz, 1H), 3.53 (s, 2H), 3.18-3.12 (m, 5H), 2.80 (s, 3H), 2.72-2.64 (m, 1H), 2.18 (d, J=10.5 Hz, 2H), 2.01-1.94 (m, 1H), 1.77-1.71 (m, 4H).

### N-{5-chloro-3-[1-(4-{4-[(1-{4-[(3R)-2,6-dioxopiperidin-3-yl]phenyl}piperidin-4-yl)methyl]-1,4-diazepan-1-yl}-2-fluorophenyl)-3-(pyridin-4-yl)-1H-pyrazol-4-yl]-2-fluorophenyl}pyrrolidine-1-sulfonamide (346)

LCMS: C₄₆H₅₀ClF₂N₉O₄S requires: 897.3, found: m/z=898.2 [M+H]⁺. ¹H NMR (500 MHz, DMSO-d₆) δ 10.77 (s, 1H), 9.93 (s, 1H), 8.63-8.49 (m, 2H), 8.39 (d, J=1.9 Hz, 1H), 8.14 (s, OH), 7.59 (s, 1H), 7.51 (dd, J=6.5, 2.7 Hz, 1H), 7.42-7.39 (m, 2H), 7.34 (dd, J=5.5, 2.7 Hz, 1H), 7.00 (d, J=8.2 Hz, 2H), 6.87 (d, J=8.3 Hz, 2H), 6.78 (s, 2H), 6.70 (s, 1H), 3.77-3.58 (m, 6H), 3.53 (s, 2H), 3.19-3.09 (m, 4H), 2.69-2.56 (m, 4H), 2.39-2.34 (m, 1H), 2.18-2.05 (m, 1H), 2.00 (dd, J=13.2, 5.0 Hz, 1H), 1.89 (s, 2H), 1.80-1.67 (m, 4H), 1.21 (d, J=28.0 Hz, 2H).

### Rac-N-{5-chloro-3-[1-(4-{4-[(1-{4-[(3R)-2,6-dioxopiperidin-3-yl]-2-fluorophenyl}piperidin-4-yl)methyl]piperazin-1-yl}phenyl)-3-(pyridin-4-yl)-1H-pyrazol-4-yl]-2-fluorophenyl}pyrrolidine-1-sulfonamide (347)

LCMS: C₄₅H₄₈ClF₂N₉O₄S requires: 883.3, found: m/z=884.2 [M+H]⁺. ¹H NMR (500 MHz, DMSO-d₆) δ 10.82 (s, 1H), 9.94 (s, 1H), 8.75 (s, 1H), 8.57 (d, J=5.2 Hz, 2H), 7.80 (s, 2H), 7.52 (dd, J=6.4, 2.7 Hz, 1H), 7.46-7.36 (m, 2H), 7.11 (s, 1H), 6.99 (d, J=22.1 Hz, 3H), 3.80 (d, J=9.1 Hz, 1H), 3.23 (s, 3H), 3.18-3.10 (m, 1H), 2.74-2.59 (m, 4H), 2.39-2.34 (m, 1H), 2.20 (d, J=8.7 Hz, OH), 2.01 (s, 1H), 1.85 (s, 2H), 1.79-1.65 (m, 3H), 1.30 (m, 3H).

### Rac-N-{5-chloro-3-[1-(4-{4-[(1-{4-[(3R)-2,6-dioxopiperidin-3-yl]-3-fluorophenyl}piperidin-4-yl)methyl]piperazin-1-yl}phenyl)-3-(pyridin-4-yl)-1H-pyrazol-4-yl]-2-fluorophenyl}pyrrolidine-1-sulfonamide (348)

LCMS: C₄₅H₄₈ClF₂N₉O₄S requires: 883.3, found: m/z=884.2 [M+H]⁺. ¹H NMR (500 MHz, DMSO-d₆) δ 10.81 (s, 1H), 9.93 (s, 1H), 8.74 (s, 1H), 8.59-8.54 (m, 2H), 7.79 (d, J=8.6 Hz, 2H), 7.52 (dd, J=6.5, 2.7 Hz, 1H), 7.46-7.38 (m, 3H), 7.14-7.04 (m, 3H), 6.72 (d, J=10.6 Hz, 2H), 3.88 (dd, J=12.4, 5.1 Hz, 1H), 3.73 (d, J=12.3 Hz, 2H), 3.37 (s, 1H), 3.34 (s, 3H), 3.36-3.27 (m, 1H), 3.22 (s, 3H), 3.18-3.12 (m, 2H), 2.72 (t, J=12.4 Hz, 3H), 2.24 (s, 2H), 2.15 (dd, J=12.7, 4.2 Hz, 1H), 1.99-1.93 (m, 1H), 1.81 (d, J=13.0 Hz, 2H), 1.77-1.71 (m, 4H), 1.22 (s, 3H).

### N-[5-(difluoromethyl)-3-[1-(4-{4-[(1-{4-[(3R)-2,6-dioxopiperidin-3-yl]phenyl}piperidin-4-yl)methyl]piperazin-1-yl}phenyl)-3-(pyridin-4-yl)-1H-pyrazol-4-yl]-2-fluorophenyl]pyrrolidine-1-sulfonamide (349)

LCMS: C₄₆H₅₀F₃N₉O₄S requires: 881.4, found: m/z=882.3 [M+H]⁺. ¹H NMR (500 MHz, DMSO-d₆) δ 10.78 (s, 1H), 9.91 (s, 1H), 8.76 (s, 1H), 8.58-8.53 (m, 2H), 7.81 (s, 2H), 7.71 (d, J=6.7 Hz, 1H), 7.51 (d, J=5.9 Hz, 1H), 7.43-7.38 (m, 2H), 7.10 (s, 1H), 7.05 (d, J=8.2 Hz, 2H), 6.90 (d, J=8.2 Hz, 2H), 3.73 (dd, J=11.0, 4.9 Hz, 1H), 3.25-3.19 (m, 5H), 3.19-3.13 (m, 5H), 2.66 (s, 2H), 2.48 (s, 2H), 2.24 (s, 1H), 2.13 (d, J=9.4 Hz, 1H), 2.05-1.98 (m, 1H), 1.83 (d, J=12.7 Hz, 2H), 1.77-1.70 (m, 4H), 1.25 (s, 3H).

### Rac-N-[5-(difluoromethyl)-3-[1-(4-{4-[(1-{5-[(3R)-2,6-dioxopiperidin-3-yl]pyridin-2-yl}piperidin-4-yl)methyl]piperazin-1-yl}phenyl)-3-(pyridin-4-yl)-1H-pyrazol-4-yl]-2-fluorophenyl]pyrrolidine-1-sulfonamide (350)

LCMS: C₄₅H₄₉F₃N₁₀O₄S requires: 882.4, found: m/z=883.3 [M+H]⁺. ¹H NMR (500 MHz, DMSO-d₆) δ 10.82 (s, 1H), 9.91 (s, 1H), 9.26 (s, 1H), 8.79 (s, 1H), 8.58-8.53 (m, 2H), 8.14 (s, 1H), 7.96 (d, J=2.5 Hz, 1H), 7.85 (s, 2H), 7.72 (dd, J=7.0, 2.1 Hz, 1H), 7.54-7.48 (m, 1H), 7.44-7.36 (m, 3H), 7.18 (s, 2H), 7.11 (s, OH), 6.84 (d, J=9.0 Hz, 1H), 4.30 (d, J=12.7 Hz, 2H), 3.93 (s, 1H), 3.74 (dd, J=12.1, 4.9 Hz, 1H), 3.65 (s, 1H), 3.21 (s, 5H), 3.19-3.13 (m, 4H), 2.84 (s, 2H), 2.69 (td, J=12.2, 6.2 Hz, 1H), 2.54 (d, J=3.8 Hz, OH), 2.19 (qd, J=12.7, 4.4 Hz, 1H), 2.01-1.94 (m, 1H), 1.82 (d, J=12.7 Hz, 2H), 1.78-1.69 (m, 4H), 1.20 (s, 3H).

### N-{5-chloro-3-[1-(6-{4-[(1-{4-[(3R)-2,6-dioxopiperidin-3-yl]phenyl}piperidin-4-yl)methyl]piperazin-1-yl}pyridin-3-yl)-3-(pyridin-4-yl)-1H-pyrazol-4-yl]-2-fluorophenyl}pyrrolidine-1-sulfonamide (351)

LCMS: C₄₄H₄₈ClFN₁₀O₄S requires: 866.3, found: m/z=867.2 [M+H]⁺.

### Rac-N-{5-chloro-3-[1-(6-{4-[(1-{5-[(3R)-2,6-dioxopiperidin-3-yl]pyridin-2-yl}piperidin-4-yl)methyl]piperazin-1-yl}pyridin-3-yl)-3-(pyridin-4-yl)-1H-pyrazol-4-yl]-2-fluorophenyl}pyrrolidine-1-sulfonamide (352)

LCMS: C₄₃H₄₇ClFN₁₁O₄S requires: 867.3, found: m/z=868.3 [M+H]⁺.

### N-{5-chloro-3-[1-(4-{4-[(1-{4-[(3R)-2,6-dioxopiperidin-3-yl]phenyl}piperidin-4-yl)methyl]piperazin-1-yl}-2,5-difluorophenyl)-3-(pyridin-4-yl)-1H-pyrazol-4-yl]-2-fluorophenyl}pyrrolidine-1-sulfonamide (353)

LCMS: C₄₅H₄₇ClF₃N₉O₄S requires: 901.3, found: m/z=902.2 [M+H]⁺.

### Rac-N-{5-chloro-3-[1-(4-{4-[(1-{5-[(3R)-2,6-dioxopiperidin-3-yl]pyridin-2-yl}piperidin-4-yl)methyl]piperazin-1-yl}-2,5-difluorophenyl)-3-(pyridin-4-yl)-1H-pyrazol-4-yl]-2-fluorophenyl}pyrrolidine-1-sulfonamide (354)

LCMS: C₄₄H₄₆ClF₃N₁₀O₄S requires: 902.3, found: m/z=903.2 [M+H]⁺.

### N-[5-chloro-3-(1-{4-[4-({1-[5-(2,4-dioxo-1,3-diazinan-1-yl)pyridin-2-yl]piperidin-4-yl}methyl)piperazin-1-yl]phenyl}-3-(pyridin-4-yl)-1H-pyrazol-4-yl)-2-fluorophenyl]pyrrolidine-1-sulfonamide (355)

LCMS: C₄₃H₄₇ClFN₁₁O₄S requires: 867.3, found: m/z=868.2 [M+H]⁺.

### N-[5-(difluoromethyl)-3-(1-{4-[4-({1-[4-(2,4-dioxo-1,3-diazinan-1-yl)phenyl]piperidin-4-yl}methyl)piperazin-1-yl]-2-fluorophenyl}-3-(pyridin-4-yl)-1H-pyrazol-4-yl)-2-fluorophenyl]pyrrolidine-1-sulfonamide (356)

LCMS: C₄₅H₄₈F₄N₁₀O₄S requires: 900.4, found: m/z=901.2 [M+H]⁺.

### Rac-N-[5-(difluoromethyl)-3-[1-(4-{4-[(1-{5-[(3R)-2,6-dioxopiperidin-3-yl]pyridin-2-yl}piperidin-4-yl)methyl]piperazin-1-yl}-2-fluorophenyl)-3-(pyridin-4-yl)-1H-pyrazol-4-yl]-2-fluorophenyl]pyrrolidine-1-sulfonamide (357)

LCMS: C₄₅H₄₈F₄N₁₀O₄S requires: 900.4, found: m/z=901.3 [M+H]⁺.

### N-[5-chloro-3-(1-{5-[4-({1-[4-(2,4-dioxo-1,3-diazinan-1-yl)phenyl]piperidin-4-yl}methyl)piperazin-1-yl]pyrimidin-2-yl}-3-(pyridin-4-yl)-1H-pyrazol-4-yl)-2-fluorophenyl]pyrrolidine-1-sulfonamide (358)

LCMS: C₄₂H₄₆ClFN₁₂O₄S requires: 868.3, found: m/z=869.3 [M+H]⁺.

### Rac-N-{5-chloro-3-[1-(5-{4-[(1-{5-[(3R)-2,6-dioxopiperidin-3-yl]pyridin-2-yl}piperidin-4-yl)methyl]piperazin-1-yl}pyrimidin-2-yl)-3-(pyridin-4-yl)-1H-pyrazol-4-yl]-2-fluorophenyl}pyrrolidine-1-sulfonamide (359)

LCMS: C₄₂H₄₆ClFN₁₂O₄S requires: 868.3, found: m/z=869.2 [M+H]⁺.

### N-{5-chloro-3-[1-(5-{4-[(1-{4-[(3R)-2,6-dioxopiperidin-3-yl]phenyl}piperidin-4-yl)methyl]piperazin-1-yl}pyrimidin-2-yl)-3-(pyridin-4-yl)-1H-pyrazol-4-yl]-2-fluorophenyl}pyrrolidine-1-sulfonamide (360)

LCMS: C₄₃H₄₇ClFN₁₁O₄S requires: 867.3, found: m/z=868.2 [M+H]⁺.

### N-[5-chloro-3-(1-{4-[4-({1-[4-(2,4-dioxo-1,3-diazinan-1-yl)phenyl]piperidin-4-yl}methyl)piperazin-1-yl]-3-fluorophenyl}-3-(pyridin-4-yl)-1H-pyrazol-4-yl)-2-fluorophenyl]pyrrolidine-1-sulfonamide (361)

LCMS: C₄₄H₄₇ClF₂N₁₀O₄S requires: 884.3, found: m/z=885.3 [M+H]⁺.

### N-[5-(difluoromethyl)-3-[1-(4-{4-[(1-{4-[(3R)-2,6-dioxopiperidin-3-yl]phenyl}piperidin-4-yl)methyl]piperazin-1-yl}-2-fluorophenyl)-3-(pyridin-4-yl)-1H-pyrazol-4-yl]-2-fluorophenyl]pyrrolidine-1-sulfonamide (362)

LCMS: C₄₆H₄₉F₄N₉O₄S requires: 899.4, found: m/z=900.3 [M+H]⁺.

### Rac-N-{5-chloro-3-[1-(4-{4-[(1-{5-[(3R)-2,6-dioxopiperidin-3-yl]pyridin-2-yl}piperidin-4-yl)methyl]piperazin-1-yl}-3-fluorophenyl)-3-(pyridin-4-yl)-1H-pyrazol-4-yl]-2-fluorophenyl}pyrrolidine-1-sulfonamide (363)

LCMS: C₄₄H₄₇ClF₂N₁₀O₄S requires: 884.3, found: m/z=885.2 [M+H]⁺.

### N-{5-chloro-3-[1-(4-{4-[(1-{4-[(3R)-2,6-dioxopiperidin-3-yl]phenyl}piperidin-4-yl)methyl]piperazin-1-yl}-3-fluorophenyl)-3-(pyridin-4-yl)-1H-pyrazol-4-yl]-2-fluorophenyl}pyrrolidine-1-sulfonamide (364)

LCMS: C₄₅H₄₈ClF₂N₉O₄S requires: 883.3, found: m/z=884.2 [M+H]⁺.

### (3R)—N-[5-chloro-3-(1-{4-[4-({1-[4-(2,4-dioxo-1,3-diazinan-1-yl)phenyl]piperidin-4-yl}methyl)piperazin-1-yl]-3-fluorophenyl}-3-(pyridin-4-yl)-1H-pyrazol-4-yl)-2-fluorophenyl]-3-fluoropyrrolidine-1-sulfonamide (365)

LCMS: C₄₄H₄₆ClF₃N₁₀O₄S requires: 902.3, found: m/z=903.2 [M+H]⁺.

### (3R)—N-{5-chloro-3-[1-(4-{4-[(1-{5-[(3RS)-2,6-dioxopiperidin-3-yl]pyridin-2-yl}piperidin-4-yl)methyl]piperazin-1-yl}-3-fluorophenyl)-3-(pyridin-4-yl)-1H-pyrazol-4-yl]-2-fluorophenyl}-3-fluoropyrrolidine-1-sulfonamide (366)

LCMS: C₄₄H₄₆ClF₃N₁₀O₄S requires: 902.3, found: m/z=903.2 [M+H]⁺.

### (3R)—N-{5-chloro-3-[1-(4-{4-[(1-{4-[(3R)-2,6-dioxopiperidin-3-yl]phenyl}piperidin-4-yl)methyl]piperazin-1-yl}-3-fluorophenyl)-3-(pyridin-4-yl)-1H-pyrazol-4-yl]-2-fluorophenyl}-3-fluoropyrrolidine-1-sulfonamide (367)

LCMS: C₄₅H₄₇ClF₃N₉O₄S requires: 901.3, found: m/z=902.3 [M+H]⁺.

### N-[5-chloro-3-(1-{4-[4-({1-[5-(2,4-dioxo-1,3-diazinan-1-yl)pyridin-2-yl]piperidin-4-yl}methyl)piperazin-1-yl]phenyl}-3-(pyridin-4-yl)-1H-pyrazol-4-yl)-2-fluorophenyl]-3-fluoroazetidine-1-sulfonamide (368)

LCMS: C₄₂H₄₄ClF₂N₁₁O₄S requires: 871.3, found: m/z=872.3 [M+H]⁺. ¹H NMR (500 MHz, DMSO-d₆) δ 10.34 (s, 1H), 8.76 (s, 1H), 8.60-8.53 (m, 2H), 8.14 (s, 1H), 8.05 (d, J=2.8 Hz, 1H), 7.79 (d, J=8.6 Hz, 2H), 7.54-7.36 (m, 4H), 7.12 (s, 2H), 6.86 (d, J=9.1 Hz, 1H), 4.29 (d, J=12.7 Hz, 2H), 4.07 (s, 2H), 3.89 (dd, J=24.4, 10.0 Hz, 2H), 3.70 (t, J=6.7 Hz, 2H), 2.82 (d, J=12.5 Hz, 1H), 2.71 (t, J=6.7 Hz, 2H), 2.64 (q, J=1.9 Hz, 1H), 2.37 (p, J=1.9 Hz, 1H), 1.81 (d, J=12.9 Hz, 2H), 1.14 (s, 2H).

### N-[5-chloro-3-(1-{4-[4-({1-[5-(2,4-dioxo-1,3-diazinan-1-yl)pyridin-2-yl]piperidin-4-yl}methyl)piperazin-1-yl]-2-fluorophenyl}-3-(pyridin-4-yl)-1H-pyrazol-4-yl)-2-fluorophenyl]-3-fluoroazetidine-1-sulfonamide (369)

LCMS: C₄₂H₄₃ClF₃N₁₁O₄S requires: 889.3, found: m/z=890.2 [M+H]⁺. ¹H NMR (500 MHz, DMSO-d₆) δ 10.34 (s, 1H), 8.62-8.54 (m, 2H), 8.45 (s, 1H), 8.14 (s, 1H), 8.05 (d, J=2.9 Hz, 1H), 7.67 (t, J=9.1 Hz, 1H), 7.56-7.45 (m, 2H), 7.45-7.40 (m, 2H), 7.36 (s, 1H), 7.06 (d, J=14.9 Hz, 1H), 6.95 (d, J=9.1 Hz, 1H), 6.85 (d, J=9.0 Hz, 1H), 5.34 (s, 1H), 4.29 (d, J=12.8 Hz, 2H), 4.05 (d, J=19.6 Hz, 2H), 3.95-3.80 (m, 2H), 3.70 (t, J=6.7 Hz, 2H), 2.83 (t, J=12.3 Hz, 2H), 2.70 (t, J=6.7 Hz, 2H), 2.66-2.62 (m, 1H), 2.39-2.35 (m, 1H), 1.81 (d, J=13.0 Hz, 2H), 1.13 (d, J=12.9 Hz, 2H).

### N-[5-chloro-3-(1-{4-[4-({1-[5-(2,4-dioxo-1,3-diazinan-1-yl)pyridin-2-yl]piperidin-4-yl}methyl)piperazin-1-yl]-2-fluorophenyl}-3-(pyridin-4-yl)-1H-pyrazol-4-yl)-2-fluorophenyl]pyrrolidine-1-sulfonamide (370)

LCMS: C₄₃H₄₆ClF₂N₁₁O₄S requires: 885.3, found: m/z=886.2 [M+H]⁺. ¹H NMR (500 MHz, DMSO-d₆) δ 10.34 (s, 1H), 9.94 (s, 1H), 8.60-8.54 (m, 2H), 8.44 (d, J=2.0 Hz, 1H), 8.05 (d, J=2.8 Hz, 1H), 7.68 (d, J=9.4 Hz, 1H), 7.54-7.45 (m, 2H), 7.45-7.39 (m, 2H), 7.35 (dd, J=5.6, 2.7 Hz, 1H), 7.05 (s, 1H), 6.94 (s, 1H), 6.85 (d, J=9.1 Hz, 1H), 4.29 (d, J=12.8 Hz, 2H), 3.70 (t, J=6.7 Hz, 2H), 3.15 (td, J=5.5, 3.3 Hz, 4H), 2.83 (t, J=12.4 Hz, 2H), 2.70 (t, J=6.7 Hz, 2H), 2.22 (s, 2H), 1.81 (d, J=13.0 Hz, 2H), 1.78-1.70 (m, 4H), 1.13 (s, 2H).

### (3R)—N-[5-chloro-3-(1-{4-[4-({1-[5-(2,4-dioxo-1,3-diazinan-1-yl)pyridin-2-yl]piperidin-4-yl}methyl)piperazin-1-yl]-2-fluorophenyl}-3-(pyridin-4-yl)-1H-pyrazol-4-yl)-2-fluorophenyl]-3-fluoropyrrolidine-1-sulfonamide (371)

LCMS: C₄₃H₄₅ClF₃N₁₁O₄S requires: 903.3, found: m/z=904.2 [M+H]⁺. ¹H NMR (500 MHz, DMSO-d₆) δ 10.34 (s, 1H), 10.08 (s, 1H), 8.61-8.51 (m, 2H), 8.43 (d, J=1.9 Hz, 1H), 8.05 (d, J=2.8 Hz, 1H), 7.68 (s, 1H), 7.57-7.45 (m, 2H), 7.45-7.37 (m, 2H), 7.34 (dd, J=5.7, 2.6 Hz, 1H), 7.06 (s, 1H), 6.95 (s, 1H), 6.86 (d, J=9.1 Hz, 1H), 4.29 (d, J=12.8 Hz, 2H), 3.70 (t, J=6.8 Hz, 2H), 3.44-3.35 (m, 2H), 2.84 (t, J=12.4 Hz, 2H), 2.71 (t, J=6.7 Hz, 2H), 2.31-1.89 (m, 2H), 1.81 (d, J=13.0 Hz, 2H), 1.14 (s, 2H).

### N-{5-chloro-3-[1-(4-{4-[(1-{5-[(3R)-2,6-dioxopiperidin-3-yl]pyridin-2-yl}piperidin-4-yl)methyl]piperazin-1-yl}phenyl)-3-(pyridin-4-yl)-1H-pyrazol-4-yl]-2-fluorophenyl}cyclopentanesulfonamide (372)

LCMS: C₄₅H₄₉ClFN₉O₄S requires: 865.3, found: m/z=866.2 [M+H]⁺.

### Rac-N-{5-chloro-3-[1-(4-{4-[(1-{5-[(3R)-2,6-dioxopiperidin-3-yl]pyridin-2-yl}piperidin-4-yl)methyl]piperazin-1-yl}phenyl)-3-(pyridin-4-yl)-1H-pyrazol-4-yl]-2-fluorophenyl}cyclopentanesulfonamide (373)

LCMS: C₄₅H₄₉ClFN₉O₄S requires: 865.3, found: m/z=866.3 [M+H]⁺.

### N-{5-chloro-3-[1-(4-{4-[(1-{4-[(3R)-2,6-dioxopiperidin-3-yl]phenyl}piperidin-4-yl)methyl]piperazin-1-yl}phenyl)-3-(pyridin-4-yl)-1H-pyrazol-4-yl]-2-fluorophenyl}cyclopentanesulfonamide (374)

LCMS: C₄₆H₅₀ClFN₈O₄S requires: 864.3, found: m/z=865.2 [M+H]⁺.

### (3R)-3-(6-{4-[(4-{4-[4-(3-{[ethyl(methyl)sulfamoyl]amino}-2,5-difluorophenyl)-3-(pyridin-4-yl)-1H-pyrazol-1-yl]phenyl}piperazin-1-yl)methyl]piperidin-1-yl}pyridin-3-yl)piperidine-2,6-dione (375)

LCMS: C₄₃H₄₈F₂N₁₀O₄S requires: 838.4, found: m/z=839.3 [M+H]⁺.

### Rac-(3R)-3-(6-{4-[(4-{4-[4-(3-{[ethyl(methyl)sulfamoyl]amino}-2,5-difluorophenyl)-3-(pyridin-4-yl)-1H-pyrazol-1-yl]phenyl}piperazin-1-yl)methyl]piperidin-1-yl}pyridin-3-yl)piperidine-2,6-dione (376)

LCMS: C₄₃H₄₈F₂N₁₀O₄S requires: 838.4, found: m/z=839.3 [M+H]⁺.

### (3R)-3-(4-{4-[(4-{4-[4-(3-{[ethyl(methyl)sulfamoyl]amino}-2,5-difluorophenyl)-3-(pyridin-4-yl)-1H-pyrazol-1-yl]phenyl}piperazin-1-yl)methyl]piperidin-1-yl}phenyl)piperidine-2,6-dione (377)

LCMS: C₄₄H₄₉F₂N₉O₄S requires: 837.4, found: m/z=838.3 [M+H]⁺.

### N-[3-(1-{4-[(3R)-4-[(1-{4-[(3R)-2,6-dioxopiperidin-3-yl]phenyl}piperidin-4-yl)methyl]-3-methylpiperazin-1-yl]phenyl}-3-(pyridin-4-yl)-1H-pyrazol-4-yl)-2-fluorophenyl]propane-1-sulfonamide (378)

LCMS: C₄₅H₅₁FN₈O₄S requires: 818.4, found: m/z=819.3 [M+H]⁺.

### (3R)-3-(6-{4-[(4-{4-[4-(3-{[ethyl(methyl)sulfamoyl]amino}-2-fluorophenyl)-3-(pyridin-4-yl)-1H-pyrazol-1-yl]-3-fluorophenyl}piperazin-1-yl)methyl]piperidin-1-yl}pyridin-3-yl)piperidine-2,6-dione (379)

LCMS: C₄₃H₄₈F₂N₁₀O₄S requires: 838.4, found: m/z=839.3 [M+H]⁺.

### (3S)-3-(6-{4-[(4-{4-[4-(3-{[ethyl(methyl)sulfamoyl]amino}-2-fluorophenyl)-3-(pyridin-4-yl)-1H-pyrazol-1-yl]-2-fluorophenyl}piperazin-1-yl)methyl]piperidin-1-yl}pyridin-3-yl)piperidine-2,6-dione (380)

LCMS: C₄₃H₄₈F₂N₁₀O₄S requires: 838.4, found: m/z=839.2 [M+H]⁺.

### Rac-(R)—N-(3-(1-(4-(4-((4-(4-(2,6-dioxopiperidin-3-yl)phenyl)piperazin-1-yl)methyl)piperidin-1-yl)phenyl)-3-(pyridin-4-yl)-1H-pyrazol-4-yl)-2-fluorophenyl)propane-1-sulfonamide (381)

LCMS: C₄₄H₄₉FN₈O₄S requires: 804.4, found: m/z=805.2 [M+H]⁺.

### N-(5-chloro-3-(1-(4-((3R,5R)-4-((1-(5-((RS)-2,6-dioxopiperidin-3-yl)pyridin-2-yl)piperidin-4-yl)methyl)-3,5-dimethylpiperazin-1-yl)-2-fluorophenyl)-3-(pyridin-4-yl)-1H-pyrazol-4-yl)-2-fluorophenyl)pyrrolidine-1-sulfonamide (382)

LCMS: C₄₆H₅₁ClF₂N₁₀O₄S requires: 912.3, found: m/z=913.2 [M+H]⁺.

### N-{5-chloro-3-[1-(5-{4-[(1-{5-[(3S)-2,6-dioxopiperidin-3-yl]pyridin-2-yl}piperidin-4-yl)methyl]piperazin-1-yl}pyridin-2-yl)-3-(pyridin-4-yl)-1H-pyrazol-4-yl]-2-fluorophenyl}pyrrolidine-1-sulfonamide (383)

LCMS: C₄₃H₄₇ClFN₁₁O₄S requires: 867.3, found: m/z=868.2 [M+H]⁺.

### N-{5-chloro-3-[1-(4-{4-[(1-{4-[(3S)-2,6-dioxopiperidin-3-yl]-3-fluorophenyl}piperidin-4-yl)methyl]piperazin-1-yl}phenyl)-3-(pyridin-4-yl)-1H-pyrazol-4-yl]-2-fluorophenyl}pyrrolidine-1-sulfonamide (384)

LCMS: C₄₅H₄₈ClF₂N₉O₄S requires: 883.3, found: m/z=884.2 [M+H]⁺.

### Rac-N-{5-chloro-3-[1-(4-{4-[(1-{4-[(3R)-2,6-dioxopiperidin-3-yl]-2,3-difluorophenyl}piperidin-4-yl)methyl]piperazin-1-yl}phenyl)-3-(pyridin-4-yl)-1H-pyrazol-4-yl]-2-fluorophenyl}pyrrolidine-1-sulfonamide (385)

LCMS: C₄₅H₄₇ClF₃N₉O₄S requires: 901.3, found: m/z=902.2 [M+H]⁺.

### Rac-N-{5-chloro-3-[1-(4-{4-[(1-{4-[(3R)-2,6-dioxopiperidin-3-yl]-2,5-difluorophenyl}piperidin-4-yl)methyl]piperazin-1-yl}phenyl)-3-(pyridin-4-yl)-1H-pyrazol-4-yl]-2-fluorophenyl}pyrrolidine-1-sulfonamide (386)

LCMS: C₄₅H₄₇ClF₃N₉O₄S requires: 901.3, found: m/z=902.2 [M+H]⁺.

### Rac-N-{5-chloro-3-[1-(4-{4-[(1-{4-[(3R)-2,6-dioxopiperidin-3-yl]-3,5-difluorophenyl}piperidin-4-yl)methyl]piperazin-1-yl}phenyl)-3-(pyridin-4-yl)-1H-pyrazol-4-yl]-2-fluorophenyl}pyrrolidine-1-sulfonamide (387)

LCMS: C₄₅H₄₇ClF₃N₉O₄S requires: 901.3, found: m/z=902.2 [M+H]⁺.

### N-[5-chloro-3-(1-{4-[4-({1-[5-(2,4-dioxo-1,3-diazinan-1-yl)pyridin-2-yl]piperidin-4-yl}methyl)piperazin-1-yl]phenyl}-3-(pyridin-4-yl)-1H-pyrazol-4-yl)-2-fluorophenyl]cyclopentanesulfonamide (388)

LCMS: C₄₄H₄₈ClFN₁₀O₄S requires: 866.3, found: m/z=867.2 [M+H]⁺.

### Cell Line Generation for HiBiT Evaluation

An A375 cell line expressing a homozygous V600E mutation was purchased from ATCC (Manassas, VA, USA). An A375 clone 1F10 cell line with an endogenous C-terminal HiBiT fusion tag on BRAF V600E allele was generated internally using CRISPR-Cas9 technology and cultured in DMEM, high glucose supplemented with 10% FBS and 1× GlutaMAX. A HCT-116 BRAF−/− cell line overexpressing the C-terminal HiBiT-tagged G466V, G469A, and D594G mutants were generated by lentiviral transduction, externally at Biosettia Inc (San Diego, CA, USA). The stably transfected cells were cultured in RPMI media supplemented with 10% FBS, 1× GlutaMAX, and 0.25 μg/ml puromycin. DMEM medium, RPMI 1640 medium, GlutaMAX, fetal bovine serum (FBS), and puromycin were purchased from Gibco (Grand Island, NY, USA). HCT-116 BRAF−/− cells were purchased from Horizon Discovery (Waterbeach, UK).

### BRAF HiBiT Degradation Assay Protocol

The C-terminal HiBiT tagged BRAF A375 cells (monoclonal cell line, cell clone 1F10) were seeded at a density of 10,000 cells/well in 30 μL media (DMEM, high glucose supplemented with 10% FBS and 1× GlutaMAX) in white opaque 384-well plates (Corning #3570) from column 1 to column 23. The wildtype A375 cells were seeded in column 24 at the same cell density in 30 μL media. Cells were incubated at 37° C. with 5% CO₂ for 24 h.

The C-terminal HiBiT tagged mutant BRAF G466V, G469A, and D594G overexpressed in HCT-116 BRAF−/− cells (monoclonal cell line, cell clones 8, 34, and 63, respectively) were seeded at the density of 3,000 cells/well in 30 μL media (RPMI supplemented with 10% FBS and 1× GlutaMAX) in white opaque 384-well plates (Corning #3570) from column 1 to column 23. Wildtype HCT-116 BRAF cells were seeded in column 24 at the same cell density in 30 μL media. Cells were incubated at 37° C. with 5% CO₂ for 24 h.

Intermediate compound dilution series (5-point, ranging from 5 mM to 0.5 μM, 10-fold dilutions in DMSO) were prepared in a source plate (Beckman Coulter cat. no. 001-16128). Final compound dilution series (11-point, ranging from 1 μM to 0.01 nM, half-log dilutions in DMSO) were stamped into the assay plate (Corning #3570, column 1-11 and 12-22) using the Echo Acoustic Liquid Handler (Beckman Coulter). Each compound was plated in duplicate, and the total volume of compound DMSO solution was 60 nL/well (0.2% final DMSO concentration). DMSO was included in wells A23-L23 and A24-L24. The DMSO-treated HiBiT-tagged samples served as Negative Control (NC) and the DMSO-treated wildtype samples served as Background Control (BC) for the HiBiT assay.

HiBiT signal was measured by a Nano-Glo HiBiT Lytic assay (Promega cat. no. N3050). HiBiT lytic detection reagent (Nano-Glo HiBiT Lytic Buffer with 1:50 Nano-Glo HiBiT Lytic Substrate and 1:100 LgBiT Protein, 40 μL) was added using Bravo. The mixture was incubated at room temperature, in the dark, and with gentle shaking for 10 min. Luminesce units (LU) were read on an EnVision plate reader 2105 (Perkin Elmer).

### HiBiT Data Analysis

Percent protein remaining and viability remaining per sample were calculated as follows:

$$\% Protein remaining = [ \frac{sample LU - average BC LU}{average NC LU - average BC LU} ] \times 100$$

% Protein remaining values were plotted as a function of compound concentration. XC₅₀ (DC₅₀) values were determined by fitting curves using the following equation:

$$Y = LowerBound + ( UpperBound - LowerBound ) / ( 1 + ( ( XC 50 / x ) \hat{} Hill )$$

For the HiBiT degradation assay, the key parameters reported include:
The dose of compound that produces 50% degradation (DC₅₀ at 50%):
   DC₅₀ at 50%: the Fitted X value (Conc.) at Y=50 (response)
Observed Dₘₐₓ (Obs. Dₘₐₓ) is the percentage protein degraded at maximal compound effect relative to DMSO-treated control:

$$Obs . {D}_{ma x} = 100 - minimum Y value ( response )$$

### Hibit Assay Results

| | Class 1 | | Class 2 | | Class 3 | | | |
|---|---|---|---|---|---|---|---|---|
| Cmpd | A375 | A375 | G469A | G469A | G466V | G466V | D594G | D594G |
| Number | Dmax (%) | DC₅₀ (nM) | Dmax (%) | DC₅₀ (nM) | Dmax (%) | DC₅₀ (nM) | Dmax (%) | DC₅₀ (nM) |
| 1 | A | B | | | | | | |
| 2 | A | C | B | B | B | B | B | B |
| 3 | A | C | B | D | B | B | B | B |
| 4 | A | B | C | D | B | B | B | D |
| 5 | B | C | | | | | | |
| 6 | A | B | C | D | B | D | B | D |
| 7 | A | B | B | D | B | C | B | D |
| 8 | A | B | B | D | B | D | B | D |
| 9 | A | C | | | | | | |
| 10 | A | B | B | D | B | B | B | D |
| 11 | B | C | | | | | | |
| 12 | B | C | | | | | | |
| 13 | A | B | | | | | | |
| 14 | B | C | | | | | | |
| 15 | A | B | C | D | B | B | B | D |
| 16 | A | B | B | C | A | B | B | A |
| 17 | B | C | | | | | | |
| 18 | A | B | B | D | B | B | B | B |
| 19 | A | B | B | D | B | B | B | B |
| 20 | A | B | | | | | | |
| 21 | A | B | B | D | B | B | B | D |
| 22 | A | B | C | D | B | C | B | D |
| 23 | A | B | C | D | B | B | B | D |
| 24 | A | B | C | D | B | B | B | D |
| 25 | A | B | C | D | B | B | B | D |
| 26 | B | C | | | | | | |
| 27 | A | A | B | C | A | B | B | B |
| 28 | A | A | B | B | B | A | B | B |
| 29 | A | B | B | B | A | A | B | A |
| 30 | A | A | B | B | B | B | B | A |
| 31 | A | B | | | | | | |
| 32 | A | A | | | | | | |
| 33 | A | B | B | A | B | A | B | B |
| 34 | A | B | | | | | | |
| 35 | A | A | C | B | B | D | B | D |
| 36 | A | A | B | B | C | D | B | D |
| 37 | B | B | | | | | | |
| 38 | A | A | C | B | C | D | B | D |
| 39 | A | A | | | | | | |
| 40 | A | A | B | B | B | D | B | D |
| 41 | A | A | B | B | B | C | B | B |
| 42 | A | A | C | D | C | D | B | D |
| 43 | A | A | B | B | B | B | B | A |
| 44 | A | A | C | D | C | D | C | D |
| 45 | A | B | B | D | A | A | B | B |
| 46 | A | A | B | D | B | A | B | C |
| 47 | A | A | B | D | A | A | B | B |
| 48 | A | A | C | D | C | D | B | D |
| 49 | A | A | B | D | B | D | B | D |
| 50 | A | B | | | | | | |
| 51 | A | A | B | D | B | C | B | C |
| 52 | A | A | C | D | C | D | C | D |
| 53 | A | A | | | | | | |
| 54 | A | A | | | | | | |
| 55 | A | B | | | | | | |
| 56 | A | A | B | B | B | A | B | A |
| 57 | A | A | B | D | B | D | B | D |
| 58 | A | B | B | D | B | C | B | D |
| 59 | A | B | B | D | A | A | B | B |
| 60 | A | B | | | | | | |
| 61 | A | A | B | A | B | D | B | D |
| 62 | A | A | B | A | B | B | B | D |
| 63 | A | A | | | | | | |
| 64 | A | B | | | | | | |
| 65 | A | A | B | D | B | D | B | D |
| 66 | A | A | | | | | | |
| 67 | B | A | C | D | C | D | B | D |
| 68 | A | B | B | C | B | C | B | D |
| 69 | A | B | C | D | B | D | B | D |
| 70 | B | C | C | D | C | D | B | D |
| 71 | B | B | | | | | | |
| 72 | B | B | C | D | B | D | B | D |
| 73 | B | C | | | | | | |
| 74 | B | B | | | | | | |
| 75 | A | B | C | D | C | D | C | D |
| 76 | B | B | C | D | C | D | C | D |
| 77 | B | B | C | D | C | D | B | D |
| 78 | A | B | | | | | | |
| 79 | B | A | | | | | | |
| 80 | A | A | C | D | B | D | B | D |
| 81 | A | B | | | | | | |
| 82 | B | B | | | | | | |
| 83 | A | A | C | C | C | C | C | C |
| 84 | A | C | C | D | C | D | B | D |
| 85 | B | B | C | D | B | D | B | D |
| 86 | B | C | | | | | | |
| 87 | A | B | B | D | B | B | B | D |
| 88 | B | B | | | | | | |
| 89 | A | B | B | D | B | D | B | D |
| 90 | B | B | | | | | | |
| 91 | B | C | | | | | | |
| 92 | B | D | C | D | C | D | C | D |
| 93 | B | B | | | | | | |
| 94 | A | A | B | D | B | A | B | A |
| 95 | B | B | | | | | | |
| 96 | A | B | B | D | B | D | B | D |
| 97 | A | B | C | D | B | D | B | D |
| 98 | B | B | | | | | | |
| 99 | B | B | | | | | | |
| 100 | A | A | C | D | C | D | B | C |
| 101 | A | B | | | | | | |
| 102 | B | B | | | | | | |
| 103 | B | B | | | | | | |
| 104 | B | C | | | | | | |
| 105 | B | D | | | | | | |
| 106 | B | C | | | | | | |
| 107 | B | B | | | | | | |
| 108 | A | A | C | D | C | D | B | D |
| 109 | A | A | C | D | C | D | C | D |
| 110 | B | A | C | D | C | D | C | D |
| 111 | B | A | C | C | C | C | B | C |
| 112 | B | A | C | D | C | D | B | D |
| 113 | A | B | | | | | | |
| 114 | A | B | C | D | C | D | B | D |
| 115 | B | C | | | | | | |
| 116 | A | B | C | D | B | D | C | D |
| 117 | A | A | C | D | C | D | B | D |
| 118 | A | A | C | D | C | D | B | D |
| 119 | A | A | C | D | C | D | B | D |
| 120 | B | C | | | | | | |
| 121 | B | C | | | | | | |
| 122 | B | | | | | | | |
| 123 | B | B | C | D | C | D | C | D |
| 124 | B | C | | | | | | |
| 125 | A | B | B | D | C | D | C | D |
| 126 | A | B | B | B | A | B | B | A |
| 127 | A | B | B | B | B | B | B | A |
| 128 | A | C | C | D | B | C | B | D |
| 129 | B | C | C | D | C | D | C | D |
| 130 | B | D | | | | | | |
| 131 | A | B | C | D | B | D | C | D |
| 132 | B | B | | | | | | |
| 133 | B | B | | | | | | |
| 134 | A | B | B | C | B | B | B | B |
| 135 | B | C | | | | | | |
| 136 | B | C | | | | | | |
| 137 | B | C | | | | | | |
| 138 | A | A | B | A | B | A | B | B |
| 139 | B | B | | | | | | |
| 140 | A | B | | | | | | |
| 141 | A | B | C | D | C | D | C | D |
| 142 | B | C | | | | | | |
| 143 | B | C | | | | | | |
| 144 | B | C | | | | | | |
| 145 | B | D | | | | | | |
| 146 | B | C | B | D | B | A | B | D |
| 147 | B | C | C | D | C | D | C | D |
| 148 | B | D | | | | | | |
| 150 | B | C | | | | | | |
| 151 | B | B | | | | | | |
| 152 | B | B | C | D | C | D | B | D |
| 153 | B | C | | | | | | |
| 154 | A | C | | | | | | |
| 155 | B | C | | | | | | |
| 156 | A | B | | | | | | |
| 157 | B | C | | | | | | |
| 158 | B | C | | | | | | |
| 159 | A | A | C | D | C | D | B | D |
| 160 | B | | | | | | | |
| 161 | B | B | | | | | | |
| 162 | B | B | | | | | | |
| 163 | B | C | C | D | C | D | C | D |
| 164 | A | A | C | D | B | D | B | D |
| 165 | B | B | C | D | C | D | C | D |
| 166 | B | C | | | | | | |
| 167 | B | B | B | D | B | D | B | D |
| 168 | A | B | | | | | | |
| 169 | B | C | | | | | | |
| 170 | B | C | C | D | C | D | C | D |
| 171 | B | C | C | D | C | D | C | D |
| 172 | B | C | | | | | | |
| 173 | A | B | C | D | B | D | B | D |
| 174 | A | B | C | D | C | D | C | D |
| 175 | B | B | | | | | | |
| 176 | B | B | | | | | | |
| 177 | A | B | C | D | C | D | C | D |
| 178 | A | A | | | | | | |
| 179 | A | A | C | D | B | D | B | A |
| 180 | A | A | B | B | B | A | B | A |
| 181 | A | A | B | D | A | A | A | A |
| 182 | A | A | | | | | | |
| 183 | A | A | B | D | B | A | B | A |
| 184 | A | A | | | | | | |
| 185 | B | B | | | | | | |
| 186 | A | B | C | D | B | D | B | D |
| 187 | A | A | B | B | B | B | B | A |
| 188 | A | A | | | | | | |
| 189 | A | A | | | | | | |
| 190 | B | B | | | | | | |
| 191 | A | A | B | D | B | B | B | D |
| 192 | A | B | C | D | B | D | B | D |
| 193 | A | A | B | D | B | B | B | A |
| 194 | A | A | C | D | B | D | B | D |
| 195 | A | A | B | A | B | A | B | A |
| 196 | A | A | | | | | | |
| 197 | A | A | B | B | B | A | B | A |
| 198 | A | A | B | D | B | D | B | A |
| 199 | A | A | | | | | | |
| 200 | A | B | | | | | | |
| 201 | B | B | | | | | | |
| 202 | A | A | | | | | | |
| 203 | A | A | B | D | A | A | B | A |
| 204 | A | A | B | D | B | D | B | D |
| 205 | A | A | B | D | B | D | B | D |
| 206 | A | A | B | D | B | B | B | A |
| 207 | A | B | B | D | B | D | B | D |
| 208 | A | B | B | D | B | D | B | D |
| 209 | A | A | B | B | A | A | A | A |
| 210 | A | A | B | C | B | A | B | A |
| 211 | A | C | C | D | B | D | B | D |
| 212 | A | A | B | B | B | A | B | A |
| 213 | A | A | | | | | | |
| 214 | B | B | | | | | | |
| 215 | A | B | C | D | C | D | C | D |
| 216 | A | A | | | | | | |
| 217 | A | B | C | D | B | D | B | D |
| 218 | A | C | | | | | | |
| 219 | A | A | B | D | B | A | B | D |
| 220 | A | A | B | D | B | B | B | A |
| 221 | A | B | B | D | B | D | B | D |
| 222 | B | D | | | | | | |
| 223 | B | C | | | | | | |
| 224 | A | A | C | D | B | D | B | D |
| 225 | A | B | C | D | B | D | B | D |
| 226 | B | C | | | | | | |
| 227 | A | A | B | D | B | D | B | D |
| 228 | A | A | | | | | | |
| 229 | B | D | | | | | | |
| 230 | B | A | | | | | | |
| 231 | B | A | | | | | | |
| 232 | B | C | | | | | | |
| 233 | B | B | | | | | | |
| 234 | A | A | C | D | B | B | B | A |
| 235 | A | A | B | D | B | D | B | D |
| 236 | A | A | | | | | | |
| 237 | A | A | | | | | | |
| 238 | A | A | B | D | B | B | B | A |
| 239 | A | A | | | | | | |
| 240 | B | D | | | | | | |
| 241 | A | A | | | | | | |
| 242 | A | A | | | | | | |
| 243 | B | C | C | D | C | D | B | D |
| 244 | A | A | C | D | B | B | B | A |
| 245 | A | A | B | D | B | D | B | A |
| 246 | A | B | C | D | C | D | B | D |
| 247 | A | B | C | D | B | D | B | D |
| 248 | A | A | | | | | | |
| 249 | B | C | | | | | | |
| 250 | A | C | C | D | B | D | B | D |
| 251 | A | A | B | D | B | A | B | A |
| 252 | A | B | C | D | C | D | C | D |
| 253 | B | B | | | | | | |
| 254 | A | A | B | B | A | A | B | A |
| 255 | C | D | | | | | | |
| 256 | B | C | | | | | | |
| 257 | A | B | C | D | C | D | C | D |
| 258 | B | A | C | D | B | D | C | D |
| 259 | B | B | C | D | C | D | C | D |
| 260 | B | B | C | D | C | D | C | D |
| 261 | A | A | B | D | B | C | B | D |
| 262 | A | A | C | D | C | D | C | D |
| 263 | A | A | B | A | A | A | B | A |
| 264 | A | A | A | A | A | A | A | A |
| 265 | A | A | A | A | A | A | A | A |
| 266 | A | A | A | B | A | A | B | A |
| 267 | A | A | A | B | A | A | A | A |
| 268 | A | A | A | A | A | A | A | A |
| 275 | A | A | B | B | A | A | B | A |
| 276 | A | A | A | A | A | A | B | A |
| 277 | A | A | B | B | A | A | B | C |
| 278 | A | A | B | D | A | A | B | C |
| 279 | A | A | B | B | A | A | B | A |
| 280 | A | B | C | D | B | D | C | D |
| 281 | A | A | B | B | A | A | B | A |
| 282 | A | A | B | A | A | A | B | A |
| 283 | A | A | B | D | A | A | B | A |
| 284 | A | B | A | B | A | A | A | A |
| 285 | A | A | A | A | A | A | B | A |
| 286 | A | A | B | B | A | A | A | A |
| 287 | A | A | B | B | B | A | B | A |
| 288 | A | A | A | A | A | A | A | A |
| 289 | A | A | A | B | A | A | A | A |
| 290 | A | A | A | A | A | A | A | A |
| 291 | A | A | A | A | A | A | A | A |
| 292 | A | A | B | C | B | A | B | A |
| 293 | A | A | A | A | A | A | A | A |
| 294 | A | A | A | A | A | A | A | A |
| 295 | A | A | B | C | B | A | B | A |
| 296 | A | A | A | A | A | A | A | A |
| 297 | A | A | A | A | A | A | A | A |
| 298 | B | C | C | D | C | D | C | D |
| 299 | A | A | B | B | A | A | B | A |
| 300 | A | B | B | C | A | A | B | A |
| 301 | A | A | A | B | A | A | B | A |
| 302 | A | A | B | B | A | A | A | A |
| 303 | A | A | B | C | B | B | B | A |
| 304 | A | A | A | B | A | A | A | A |
| 305 | A | A | A | A | A | A | A | A |
| 306 | A | A | B | B | A | A | B | A |
| 307 | A | A | B | C | A | A | B | B |
| 308 | A | A | A | B | A | A | A | A |
| 309 | A | A | A | B | A | A | B | A |
| 310 | A | A | B | B | A | B | A | A |
| 311 | A | B | B | D | B | C | B | B |
| 312 | A | A | B | A | A | A | A | A |
| 313 | A | B | B | C | A | B | B | A |
| 314 | A | A | B | B | A | B | A | A |
| 315 | A | A | B | B | A | A | B | A |
| 316 | A | A | A | B | A | A | B | A |
| 317 | A | A | A | A | A | A | B | A |
| 318 | A | A | A | A | A | A | A | A |
| 319 | A | A | B | C | A | A | B | A |
| 320 | A | A | A | A | A | A | A | A |
| 321 | A | A | A | A | A | A | A | A |
| 322 | A | A | B | C | B | A | B | A |
| 323 | A | A | A | A | A | A | A | A |
| 324 | A | A | B | C | B | A | B | A |
| 325 | A | A | A | B | A | A | A | A |
| 326 | A | A | B | B | A | A | A | A |
| 327 | A | A | A | B | A | A | A | A |
| 328 | A | A | B | C | A | B | B | A |
| 329 | A | A | A | B | A | A | A | A |
| 330 | A | A | B | B | A | A | B | A |
| 331 | A | A | A | A | A | A | B | A |
| 332 | A | A | A | A | A | A | B | A |
| 333 | A | A | B | A | A | A | B | A |
| 334 | A | A | A | B | A | A | A | A |
| 335 | A | A | A | B | A | A | A | A |
| 336 | A | A | A | B | A | A | A | A |
| 337 | A | A | B | B | A | A | B | A |
| 338 | A | A | B | A | A | A | B | A |
| 339 | A | A | C | D | B | A | B | B |
| 340 | A | A | | | | | | |
| 341 | A | A | | | | | | |
| 342 | A | A | | | | | | |
| 343 | A | A | | | | | | |
| 344 | A | A | A | A | A | A | A | A |
| 345 | A | A | C | D | B | D | C | D |
| 346 | A | A | C | D | B | D | C | D |
| 347 | A | A | B | B | A | A | A | A |
| 348 | A | A | B | B | A | A | B | A |
| 349 | A | A | A | A | A | A | A | A |
| 350 | A | A | A | A | A | A | A | A |
| 351 | A | A | B | B | A | A | B | A |
| 352 | A | A | A | A | A | A | B | A |
| 353 | A | A | A | A | A | A | A | A |
| 354 | A | A | B | B | A | A | B | A |
| 355 | A | A | B | B | A | A | B | A |
| 356 | A | B | C | D | B | B | B | D |
| 357 | A | A | A | B | A | A | B | A |
| 358 | A | A | B | D | B | B | B | A |
| 359 | A | A | A | A | A | A | A | A |
| 360 | A | A | B | A | A | A | A | A |
| 361 | A | A | B | D | A | A | B | A |
| 362 | A | A | B | B | A | A | B | A |
| 363 | A | A | A | A | A | A | A | A |
| 364 | A | B | A | B | A | A | A | A |
| 365 | A | A | B | D | B | A | B | A |
| 366 | A | A | A | A | A | A | A | A |
| 367 | A | A | A | A | A | A | A | A |
| 368 | A | A | B | B | B | A | B | A |
| 369 | A | A | B | A | B | B | B | A |
| 370 | A | A | B | D | B | A | B | A |
| 371 | A | A | B | A | B | A | B | A |
| 372 | A | A | A | B | A | B | A | A |
| 373 | A | A | A | B | A | A | A | A |
| 374 | A | A | B | B | A | B | B | B |
| 375 | A | A | A | B | A | A | A | A |
| 376 | A | A | B | B | A | A | A | A |
| 377 | A | B | B | A | A | B | B | B |
| 378 | A | B | B | D | B | B | B | D |
| 379 | A | C | B | D | B | C | B | C |
| 380 | A | C | B | D | B | C | B | B |
| 381 | A | B | B | B | B | B | B | A |
| 382 | A | A | A | B | A | A | A | A |
| 383 | A | A | A | B | A | A | A | A |
| 384 | A | B | B | D | B | B | B | D |
| 385 | A | B | B | C | A | A | B | A |
| 386 | A | B | B | C | A | B | B | B |
| 387 | A | B | B | B | A | A | B | A |
| 388 | A | A | B | B | A | A | B | A |

DC₅₀ (nM): D > 500; 50 < C < 500; 10 < B < 50; A < 10
Dmax (%): C < 30; 30 < B < 70, A > 70

### Other Embodiments

It is to be understood that the foregoing description is intended to illustrate and not limit the scope of this disclosure, which is defined by the scope of the appended claims. Other aspects, advantages, and modifications are within the scope of the following claims.

LENGTHY TABLES
The patent application contains a lengthy table section. A copy of the table is available in electronic form from the USPTO web site (https://seqdata.uspto.gov/docdetail?docId=US20260317324A1). An electronic copy of the table will also be available from the USPTO upon request and payment of the fee set forth in 37 CFR 1.19(b)(3).

What is claimed is:

1. A compound of Formula (I) 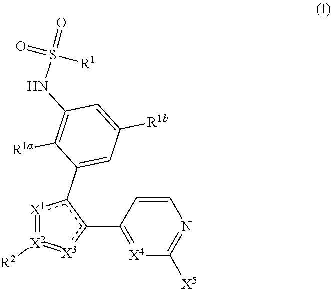
wherein
R¹ is alkyl, aryl, amino, alkylamino, dialkylamino, cycloalkyl, or heterocycloalkyl, each unsubstituted or substituted with one or more halogen or hydroxyl;
R^{1a} is halogen;
R^{1b} is hydrogen, haloalkyl, or halogen;
R² is hydrogen, alkyl, cycloalkyl, haloalkyl, or —[Ar¹—Z¹]ₙ—Z²-Cy¹—Z³-L-UBM;
X¹ is carbon or nitrogen;
X² is carbon or nitrogen;
X³ is sulfur, nitrogen, oxygen, or carbon;
the bonds among X¹, X², and X³ comprise single and double bonds, and the ring containing X¹, X², and X³ is aromatic;
X⁴ is carbon or nitrogen;
X⁵ is hydrogen or —NR³R⁴;
R³ is hydrogen or alkyl where alkyl is unsubstituted or substituted with hydroxyl;
R⁴ is hydrogen, alkyl, cycloalkyl, or —[Ar¹—Z¹]ₙ—Z²-Cy¹—Z³-L-UBM wherein
Ar¹ is arylene or heteroarylene, each unsubstituted or substituted with one or more alkyl or one or more halogen;
Z¹ is a bond or —CH₂—;
n is zero or one;
Z² is absent, —C(O)—, —O—, C₁₋₁₀ alkylene, C₁₋₁₀ alkylene-C(Me)(Me)-, three- to six-membered cycloalkylene, three- to six-membered cycloalkylene-C₁₋₁₀ alkylene, heterocycloalkylene, or —C(Me)(Me)-;
Cy¹ is absent or heterocycloalkylene, unsubstituted or substituted with one or more alkyl or one or more halogen;
Z³ is absent, C₁₋₁₀ alkylene, or —C(O)—;
L is a linker according to -L¹-L²-L³-L⁴- or -L⁴-L³-L²-L¹-, wherein
-L¹- is absent, —N(R¹⁰)—, —C(R¹¹)₂—, —C(O)—, —C₁₋₈ alkylene-, —C₂₋₈ alkynylene-, —C₆₋₁₀ aryl-, —C₄₋₁₀ heteroaryl-, -Q¹-, or -Q²-;
each -L²-, -L³-, and -L⁴- is independently, absent, —N(R¹⁰)—, —C(R¹¹)₂—, —C(O)—, —O—, —(CH₂CH₂—O)₁₋₈—, —C₁₋₈ alkylene-, —C₂₋₈ alkynylene-, —C₆₋₁₀ aryl-, —C₄₋₁₀ heteroaryl-, -Q¹-, -Q²-, or -Q³-;
each R¹⁰ is independently, hydrogen or methyl;
each R¹¹ is, independently, hydrogen, methyl, aryl, or heteroaryl;
each -Q¹- is a three- to seven-membered heterocycloalkylene comprising at least one nitrogen and is unsubstituted or substituted with one or more methyl, hydroxyl, or halogen;
each -Q²- is a five- to thirteen-membered bicyclic heterocycloalkylene comprising at least one nitrogen, wherein the five- to thirteen-membered bicyclic heterocycloalkylene is optionally a spiro bicyclic heterocycloalkylene ring;
each -Q³- is a three- to six-membered cycloalkylene;
UBM is a ubiquitin ligase binding moiety;
wherein at least one of R² or R⁴ is —[Ar¹—Z¹]ₙ—Z²-Cy¹—Z³-L-UBM; or
a stereoisomer and/or pharmaceutical salt thereof.

2. The compound of claim 1, having the following Formula (I) 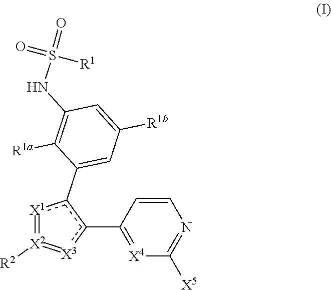
wherein
R¹ is alkyl, aryl, amino, alkylamino, dialkylamino, cycloalkyl, or heterocycloalkyl, each unsubstituted or substituted with one or more halogen or hydroxyl;
R^{1a} is halogen;
R^{1b} is hydrogen or halogen;
R² is hydrogen, alkyl, cycloalkyl, haloalkyl, or —[Ar¹—Z¹]ₙ—Z²-Cy¹—Z³-L-UBM;
X¹ is carbon or nitrogen;
X² is carbon or nitrogen;
X³ is sulfur, nitrogen, oxygen, or carbon;
the bonds among X¹, X², and X³ comprise single and double bonds, and the ring containing X¹, X², and X³ is aromatic;
X⁴ is carbon or nitrogen;
X⁵ is hydrogen or —NR³R⁴;
R³ is hydrogen or alkyl where alkyl is unsubstituted or substituted with hydroxyl;
R⁴ is hydrogen, alkyl, cycloalkyl, or —[Ar¹—Z¹]ₙ—Z²-Cy¹—Z³-L-UBM wherein
Ar¹ is arylene or heteroarylene, each unsubstituted or substituted with one or more alkyl or one or more halogen;
Z¹ is a bond or —CH₂—;
n is zero or one;
Z² is absent, —C(O)—, —O—, C₁₋₁₀ alkylene, C₁₋₁₀ alkylene-C(Me)(Me)-, three- to six-membered cycloalkylene, three- to six-membered cycloalkylene-C₁₋₁₀ alkylene, heterocycloalkylene, or —C(Me)(Me)-;
Cy¹ is absent or heterocycloalkylene, unsubstituted or substituted with alkyl;
Z³ is absent, C₁₋₁₀ alkylene, or —C(O)—;
L is a linker according to -L¹-L²-L³-L⁴- or -L⁴-L³-L²-L¹-, wherein
-L¹- is absent, —N(R¹⁰)—, —C(R¹¹)₂—, —C(O)—, —C₁₋₈ alkylene-, —C₂₋₈ alkynylene-, —C₆₋₁₀ aryl-, —C₄₋₁₀ heteroaryl-, -Q¹-, or -Q²-;
each -L²-, -L³-, and -L⁴- is independently, absent, —N(R¹⁰)—, —C(R¹¹)₂—, —C(O)—, —O—, —(CH₂CH₂—O)₁₋₈—, —C₁₋₈ alkylene-, —C₂₋₈ alkynylene-, —C₆₋₁₀ aryl-, —C₄₋₁₀ heteroaryl-, -Q¹-, -Q²-, or -Q³-;
each R¹⁰ is independently, hydrogen or methyl;
each R¹¹ is, independently, hydrogen, methyl, aryl, or heteroaryl;
each -Q¹- is a three- to seven-membered heterocycloalkylene comprising at least one nitrogen and is unsubstituted or substituted with methyl, hydroxyl, or halogen;
each -Q²- is a five- to thirteen-membered bicyclic heterocycloalkylene comprising at least one nitrogen, wherein the five- to thirteen-membered bicyclic heterocycloalkylene is optionally a spiro bicyclic heterocycloalkylene ring;
each -Q³- is a three- to six-membered cycloalkylene;
UBM is a ubiquitin ligase binding moiety;
wherein at least one of R² or R⁴ is —[Ar¹—Z¹]n-Z²-Cy¹—Z³-L-UBM; or
a stereoisomer and/or pharmaceutical salt thereof.

3. The compound of claim 2, wherein X¹ is nitrogen; X² is carbon, X³ is sulfur; and X⁴ is carbon or nitrogen.

4. The compound of claim 3, wherein
R¹ is alkyl;
R^{1a} is fluoro;
R² is —[Ar¹—Z¹]ₙ—Z²-Cy¹—Z³-L-UBM;
n is zero;
Z² is absent; and
Cy¹ is an unsubstituted heterocycloalkylene.

5. The compound of claim 4, wherein Z³ is —C(O)—.

6. The compound of claim 5, wherein L¹ is Q¹.

7. The compound of claim 6, wherein L² is —C(O)—.

8. The compound of claim 7, wherein L³ is Q¹; and L⁴ is absent.

9. The compound of any one of claims 4-8, wherein R¹ is —CH₂CH₂CH₃.

10. The compound of claim 2, wherein X¹ is carbon or nitrogen; X² is nitrogen, and X³ is carbon or nitrogen.

11. The compound of claim 10, wherein X¹ is carbon; X² is nitrogen, and X³ is nitrogen.

12. The compound of claim 11, wherein
R¹ is alkyl;
R^{1a} is fluoro;
R² is —[Ar¹—Z¹]ₙ—Z²-Cy¹—Z³-L-UBM;
Ar¹ is 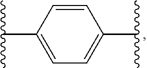 [SMILES: *c1ccc(*)cc1]
Z¹ is a bond;
n is one;
Z² and Z³ are absent; and
Cy¹ is an unsubstituted heterocycloalkylene.

13. The compound of claim 12, wherein X⁴ is carbon and X⁵ is hydrogen.

14. The compound of claim 13, wherein L¹ is —C(R¹¹)₂—.

15. The compound of claim 14, wherein is R¹¹ hydrogen.

16. The compound of claim 15, wherein L² is Q¹; and L³ and L⁴ are absent.

17. The compound of any one of claims 11-16, wherein R¹ is —CH₂CH₂CH₃.

18. The compound of claim 2, wherein X¹ is nitrogen; X² is carbon, X³ is sulfur; and X⁴ is nitrogen.

19. The compound of claim 18, wherein
R¹ is alkyl;
R^{1a} is fluoro;
R² is alkyl;
n is zero;
Z² is absent; and
Cy¹ is an unsubstituted heterocycloalkylene.

20. The compound of claim 19, wherein Z³ is absent, —CH₂— or —C(O)—.

21. The compound of claim 20, wherein L¹ is Q¹, —C(R¹¹)₂—, or —CH₂CH₂CH₂CH₂CH₂—, or Q³.

22. The compound of claim 21, wherein L² is absent, Q¹, Q², —C(R¹¹)₂—, —C(O)—, or —O—.

23. The compound of claim 22, wherein L³ is absent, Q¹, or —CH₂CH₂CH₂—; and L⁴ is absent.

24. The compound of any one of claims 19-23, wherein R¹ is —CH₂CH₂CH₃.

25. The compound of claim 2, wherein UBM binds an E3 ubiquitin ligase.

26. The compound of claim 2, wherein UBM binds SCFβ-TRCP, VHL, MDM2, IAP, or CRBN.

27. The compound of claim 2, wherein UBM binds VHL.

28. The compound of claim 27, wherein UBM binds VHL and has the following chemical formula 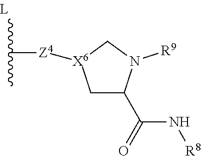
wherein 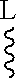
designates attachment to L;
Z⁴ is a bond, —CH₂—, —C(O)—, —C(O)N(R¹²)—, or —N(R¹²)—, wherein R¹² is hydrogen or methyl;
X⁶ is C—H or nitrogen;
R⁸ is alkyl, alkenyl, alkylene, alkynyl, aryl, cycloalkyl, heterocycloalkyl, heteroaryl, —S(O)(R¹³), or —S(O)₂(R¹³); wherein R¹³ is hydrogen, hydroxyl, alkyl, alkenyl, alkynyl, aryl, heterocycle, or heteroaryl, wherein each alkyl, alkenyl, alkylene, alkynyl, aryl, cycloalkyl, heterocycloalkyl, and heteroaryl is unsubstituted or substituted;
R⁹ is hydrogen, unsubstituted or substituted C₁₋₈ alkyl, or -AA¹-AA²-R¹⁴ wherein each AA¹ and AA² is an amino acid residue, and R¹⁴ is hydrogen or methyl.

29. The compound of claim 27, wherein UBM binds VHL and has the following chemical formula 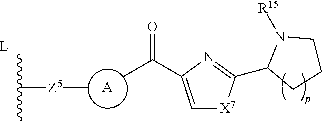
wherein 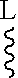
designates attachment to L;
Z⁵ is a bond, —CH₂—, —C(O)—, —C(O)N(R¹²)—, —N(R¹²)—, or —O—, wherein R¹² is hydrogen or methyl;
A is phenyl or C₄-heteroaryl;
X⁷ is —CH₂, —NR¹², oxygen, or sulfur, wherein R¹² is hydrogen or methyl;
p is zero or one;
R¹⁵ is unsubstituted or substituted C₁₋₈ alkyl, -AA¹-AA²-R¹⁴, wherein each AA¹ and AA² is an amino acid residue, and R¹⁴ is hydrogen or methyl.

30. The compound of claim 27, wherein UBM binds VHL and has the following chemical formula 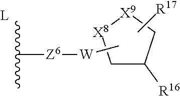
wherein 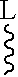
designates attachment to L;
Z⁶ is a bond, —CH₂—, —C(O)—, —C(O)N(R¹²)—, or N(R¹²), wherein R¹² is hydrogen or methyl;
W is 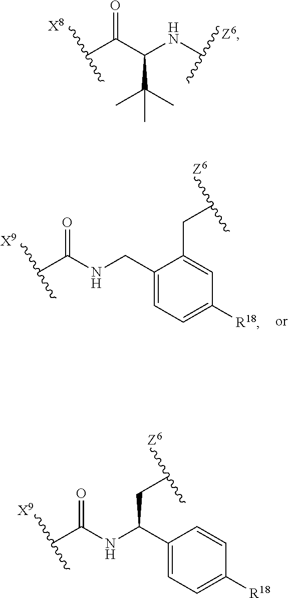
wherein 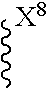
designates attachment to X⁸, wherein 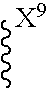
designates attachment to X⁹, and wherein, 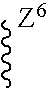
designates attachment to Z⁶;
X⁸ is carbon or nitrogen;
X⁹ is C—H or —CH₂—;
R¹⁶ is hydrogen, —OH, halogen, —NH₂, —C₁₋₃ alkyl, —C₂₋₆ alkenyl, —C₂₋₆ alkynyl, —C₁₋₃ alkoxy, —C₁₋₃ thioalkyl, —C₁₋₃ alkylamine, —C₆₋₁₀ aryl, cycloalkyl, heterocycloalkyl, or heteroaryl;
R¹⁷ is —C(O)N(H)—C₆₋₁₀ aralkyl, —C(O)CH(t-butyl)-N(H)—C₃₋₆ cycloalkyl, —C(O)—CH(t-butyl)-N(H)C(O)—C₃-C₆ cycloalkyl, 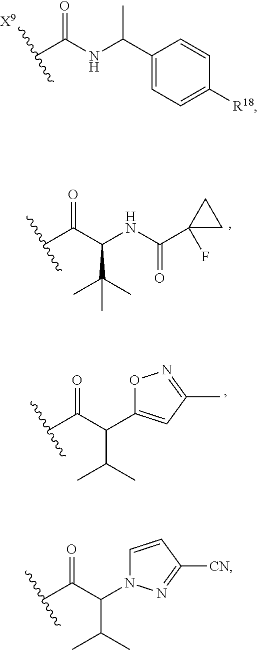
or -AA¹-AA²-R¹⁴, wherein each AA¹ and AA² is an amino acid residue and R¹⁴ is hydrogen or methyl; and
R¹⁸ is halogen or

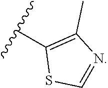 [SMILES: *c1scnc1C]

31. The compound of claim 2, wherein UBM binds CRBN.

32. The compound of claim 31, wherein UBM binds CRBN and has the following chemical formula 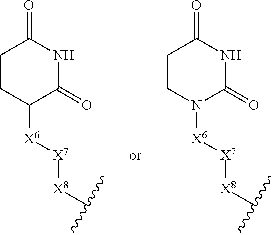
wherein
X⁶ absent or NR³;
X⁷ absent or —C(O)—; and
X⁸ is arylene or heteroarylene.

33. The compound of claim 32, wherein UBM binds CRBN and is selected from the group consisting of 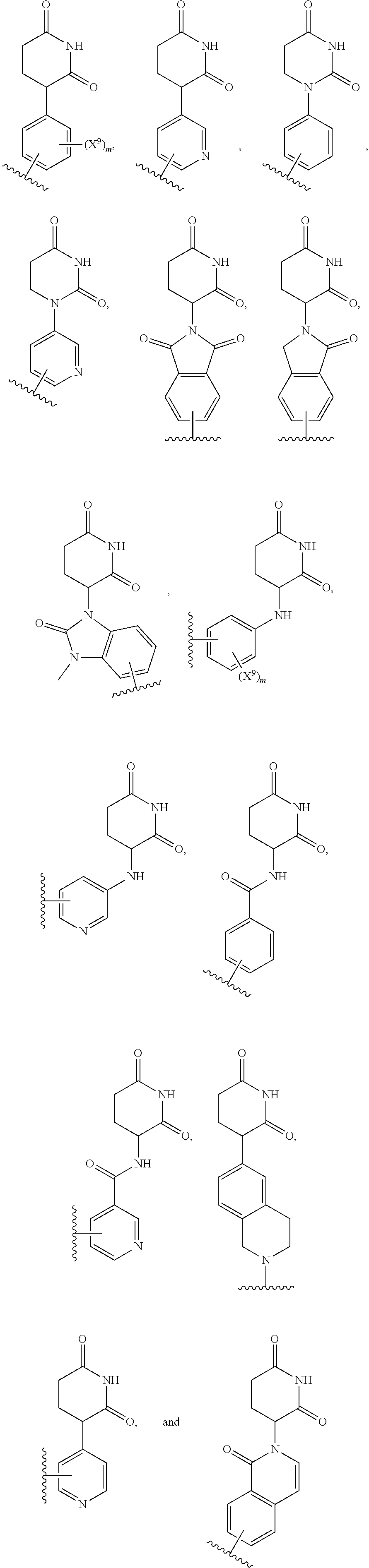
wherein
X⁹ is absent or halogen; and
m is one, two, three, or four.

34. A compound of Formula (II) 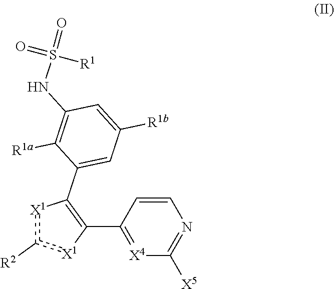
wherein
R¹ is alkyl, aryl, amino, alkylamino, dialkylamino, cycloalkyl, or heterocycloalkyl, each unsubstituted or substituted with one or more halogen or hydroxyl;
R^{1a} is halogen;
R^{1b} is hydrogen or halogen;
R² is —[Ar¹—Z¹]ₙ—Z²-Cy¹—Z³-L-UBM wherein
Ar¹ is arylene or heteroarylene, each unsubstituted or substituted with one or more alkyl or one or more halogen;
Z¹ is a bond or —CH₂—;
n is zero or one;
Z² is absent, —C(O)—, —O—, C₁₋₁₀ alkylene, C₁₋₁₀ alkylene-C(Me)(Me)-, three- to six-membered cycloalkylene, three- to six-membered cycloalkylene-C₁₋₁₀ alkylene, heterocycloalkylene, or —C(Me)(Me)-;
Cy¹ is absent or heterocycloalkylene, unsubstituted or substituted with alkyl;
Z³ is absent, C₁₋₁₀ alkylene, or —C(O)—;
L is a linker according to -L¹-L²-L³-L⁴- or -L⁴-L³-L²-L¹-, wherein
-L¹- is absent, —N(R¹⁰)—, —C(R¹¹)₂—, —C(O)—, —C₁₋₈ alkylene-, —C₂₋₈ alkynylene-, —C₆₋₁₀ aryl-, —C₄₋₁₀ heteroaryl-, -Q¹-, or -Q²-;
each -L²-, -L³-, and -L⁴- is independently, absent, —N(R¹⁰)—, —C(R¹¹)₂—, —C(O)—, —O—, —(CH₂CH₂—O)₁₋₈—, —C₁₋₈ alkylene-, —C₂₋₈ alkynylene-, —C₆₋₁₀ aryl-, —C₄₋₁₀ heteroaryl-, -Q¹-, -Q²-, or -Q³-;
each R¹⁰ is independently, hydrogen or methyl;
each R¹¹ is, independently, hydrogen, methyl, aryl, or heteroaryl;
each -Q¹- is a three- to seven-membered heterocycloalkylene comprising at least one nitrogen and is unsubstituted or substituted with methyl, hydroxyl, or halogen;
each -Q²- is a five- to thirteen-membered bicyclic heterocycloalkylene comprising at least one nitrogen, wherein the five- to thirteen-membered bicyclic heterocycloalkylene is optionally a spiro bicyclic heterocycloalkylene ring;
each -Q³- is a three- to six-membered cycloalkylene;
UBM is a ubiquitin ligase binding moiety;
X¹ is nitrogen or oxygen;
X³ is sulfur when X¹ is nitrogen; oxygen when X¹ is nitrogen; or nitrogen when X¹ is oxygen;
the bonds among X¹ and X³ comprise single and double bonds, and the ring containing X¹ and X³ is aromatic;
X⁴ is carbon or nitrogen;
X⁵ is hydrogen or —NR³R⁴;
R³ is hydrogen or alkyl where alkyl is unsubstituted or substituted with hydroxyl;
R⁴ is hydrogen, alkyl, or cycloalkyl; or
a stereoisomer and/or pharmaceutical salt thereof.

35. The compound of claim 34, wherein X¹ is nitrogen; X³ is oxygen; and X⁴ is carbon or nitrogen.

36. The compound of claim 34, wherein X¹ is oxygen; X³ is nitrogen; and X⁴ is carbon or nitrogen.

37. The compound of claim 34, wherein X¹ is nitrogen; X³ is sulfur; and X⁴ is carbon or nitrogen.

38. The compound of claim 37, wherein
R¹ is alkyl;
R^{1a} is fluoro;
R² is —[Ar¹—Z¹]ₙ—Z²-Cy¹—Z³-L-UBM;
n is zero;
Z² is absent; and
Cy¹ is an unsubstituted heterocycloalkylene.

39. The compound of claim 38, wherein Z³ is —C(O)—.

40. The compound of claim 39, wherein L¹ is Q¹.

41. The compound of claim 40, wherein L² is —C(O)—.

42. The compound of claim 41, wherein L³ is Q¹ and L⁴ is absent.

43. The compound of any one of claims 38-42, wherein R¹ is —CH₂CH₂CH₃.

44. The compound of claim 34, wherein UBM binds an E3 ubiquitin ligase.

45. The compound of claim 34, wherein UBM binds SCFP-TRCP, VHL, MDM2, IAP, or CRBN.

46. The compound of claim 45, wherein UBM binds VHL.

47. The compound of claim 46, wherein UBM binds VHL and has the following chemical formula 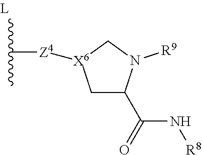
wherein 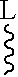
designates attachment to L;
Z⁴ is a bond, —CH₂—, —C(O)—, —C(O)N(R¹²)—, or —N(R¹²)—, wherein R¹² is hydrogen or methyl;
X⁶ is C—H or nitrogen;
R⁸ is alkyl, alkenyl, alkylene, alkynyl, aryl, cycloalkyl, heterocycloalkyl, heteroaryl, —S(O)(R¹³), or —S(O)₂(R¹³); wherein R¹³ is hydrogen, hydroxyl, alkyl, alkenyl, alkynyl, aryl, heterocycle, or heteroaryl, wherein each alkyl, alkenyl, alkylene, alkynyl, aryl, cycloalkyl, heterocycloalkyl, and heteroaryl is unsubstituted or substituted;
R⁹ is hydrogen, unsubstituted or substituted C₁₋₈ alkyl, or -AA¹-AA²-R¹⁴ wherein each AA¹ and AA² is an amino acid residue, and R¹⁴ is hydrogen or methyl.

48. The compound of claim 46, wherein UBM binds VHL and has the following chemical formula 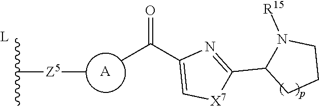
wherein 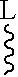
designates attachment to L;
Z⁵ is a bond, —CH₂—, —C(O)—, —C(O)N(R¹²)—, —N(R¹²)—, or —O—, wherein R¹² is hydrogen or methyl;
A is phenyl or C₄-heteroaryl;
X⁷ is —CH₂, —NR¹², oxygen, or sulfur, wherein R¹² is hydrogen or methyl;
p is zero or one;
R¹⁵ is unsubstituted or substituted C₁₋₈ alkyl, -AA¹-AA²-R¹⁴, wherein each AA¹ and AA² is an amino acid residue, and R¹⁴ is hydrogen or methyl.

49. The compound of claim 46, wherein UBM binds VHL and has the following chemical formula 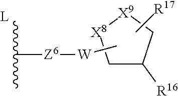
wherein 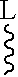
designates attachment to L;
Z⁶ is a bond, —CH₂—, —C(O)—, —C(O)N(R¹²)—, or N(R¹²), wherein R¹² is hydrogen or methyl;
W is 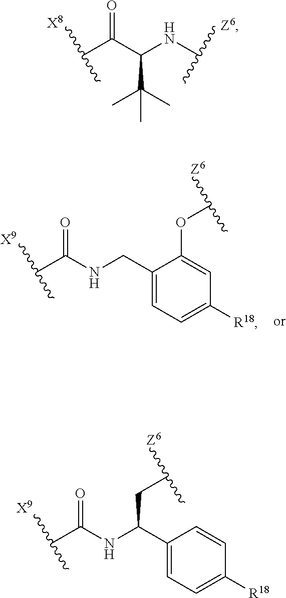
wherein 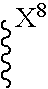
designates attachment to X⁸, wherein 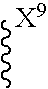
designates attachment to X⁹, and wherein, 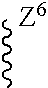
designates attachment to Z⁶;
X⁸ is carbon or nitrogen;
X⁹ is C—H or —CH₂—;
R¹⁶ is hydrogen, —OH, halogen, —NH₂, —C₁₋₃ alkyl, —C₂₋₆ alkenyl, —C₂₋₆ alkynyl, —C₁₋₃ alkoxy, —C₁₋₃ thioalkyl, —C₁₋₃ alkylamine, —C₆₋₁₀ aryl, cycloalkyl, heterocycloalkyl, or heteroaryl;
R¹⁷ is —C(O)N(H)—C₆₋₁₀ aralkyl, —C(O)CH(t-butyl)-N(H)—C₃₋₆ cycloalkyl, —C(O)—CH(t-butyl)-N(H)C(O)—C₃-C₆ cycloalkyl, 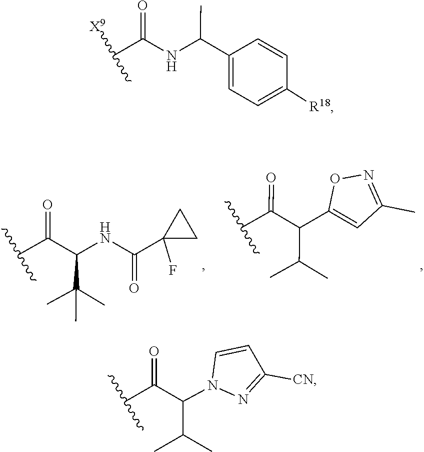
or -AA¹-AA²-R¹, wherein each AA¹ and AA² is an amino acid residue and R¹⁴ is hydrogen or methyl; and
R¹⁸ is halogen or

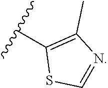 [SMILES: *c1scnc1C]

50. The compound of claim 34, wherein UBM binds CRBN.

51. The compound of claim 50, wherein UBM binds CRBN and has the following chemical formula 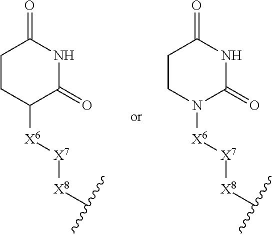
wherein
X⁶ is absent or NR³;
X⁷ is absent or —C(O)—; and
X⁸ is arylene or heteroarylene.

52. The compound of claim 51, wherein UBM binds CRBN and is selected from the group consisting of 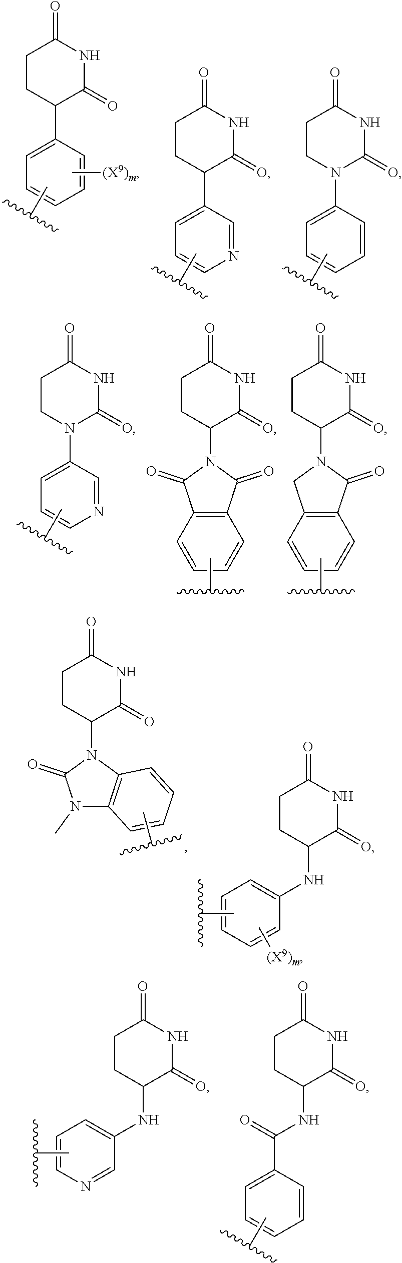 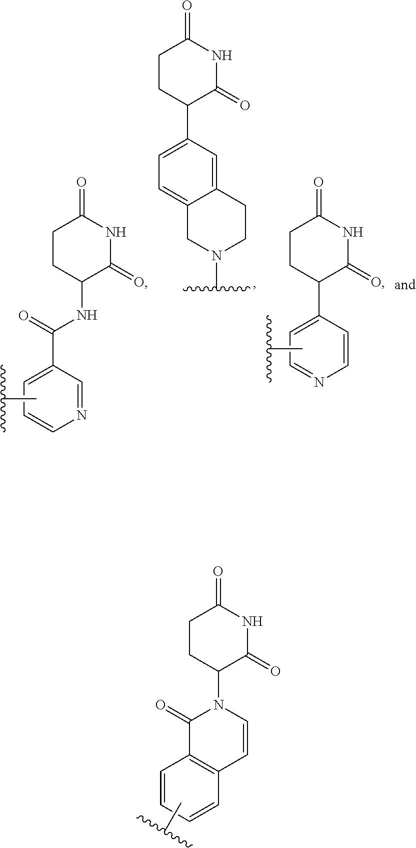
wherein
X⁹ is absent or halogen; and
m is one, two, three, or four.

53. The compound of claim 2 Formula (I) or claim 34 Formula (II), wherein L comprises at least one -Q¹-; at least one -Q²-; at least one -Q³-; at least one —C(R¹¹)₂—; or at least one C₁₋₈ alkylene-.

54. The compound of claim 53, wherein L is selected from the group consisting of

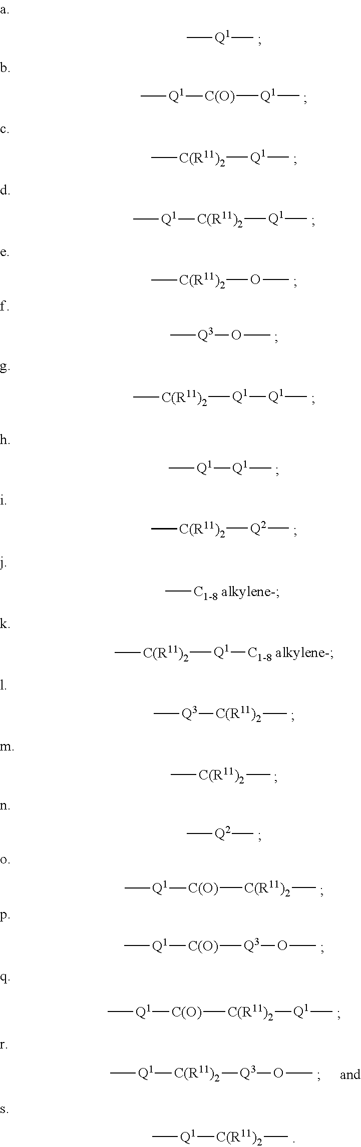

55. The compound of claim 54, wherein -Q¹- is 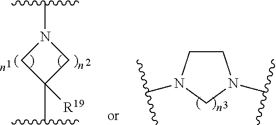
wherein R¹⁹ is hydrogen, hydroxyl, halogen, or unsubstituted alkyl, n¹ is one or two, n² is one or two, and n³ is one or two.

56. The compound of claim 55, wherein -Q¹- is selected from the group consisting of 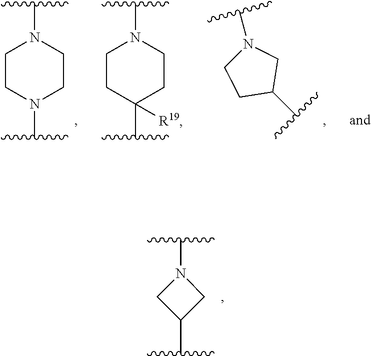
wherein R¹⁹ is hydrogen, hydroxyl, fluoro, or methyl.

57. The compound of claim 54, wherein -Q²- is 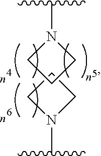
wherein n⁴ is one or two, n⁵ is one or two, and n⁶ is one or two.

58. The compound of claim 57, wherein -Q²- is selected from the group consisting of

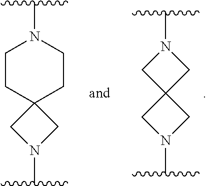 [SMILES: *N1CCC2(CC1)CN(*)C2 ; *N1CC2(C1)CN(*)C2]

59. The compound of claim 54, wherein -Q³- is 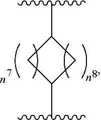
wherein n⁷ is one or two, and n⁸ is one or two.

60. The compound of claim 59, wherein -Q³- is selected from the group consisting of

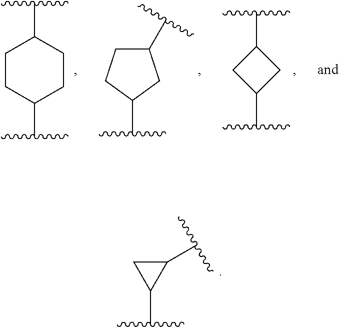

61. The compound of claim 54, wherein each R¹¹ is, independently, hydrogen or methyl.

62. The compound of claim 61, wherein both R¹¹ are hydrogen; one R¹¹ is hydrogen and one R¹¹ is methyl; or both R¹¹ are methyl.

63. The compound of claim 54, wherein —C₁₋₈ alkylene- is selected from the group consisting of —CH₂—, —CH₂CH₂—, —CH₂CH₂CH₂—, —CH₂CH₂CH₂CH₂—, —CH₂CH₂CH₂CH₂CH₂—, —CH₂CH₂CH₂CH₂CH₂CH₂—, —CH₂CH₂CH₂CH₂CH₂CH₂CH₂—, and —CH₂CH₂CH₂CH₂CH₂CH₂CH₂CH₂—.

64. The compound of claim 54, wherein the linker L is selected from 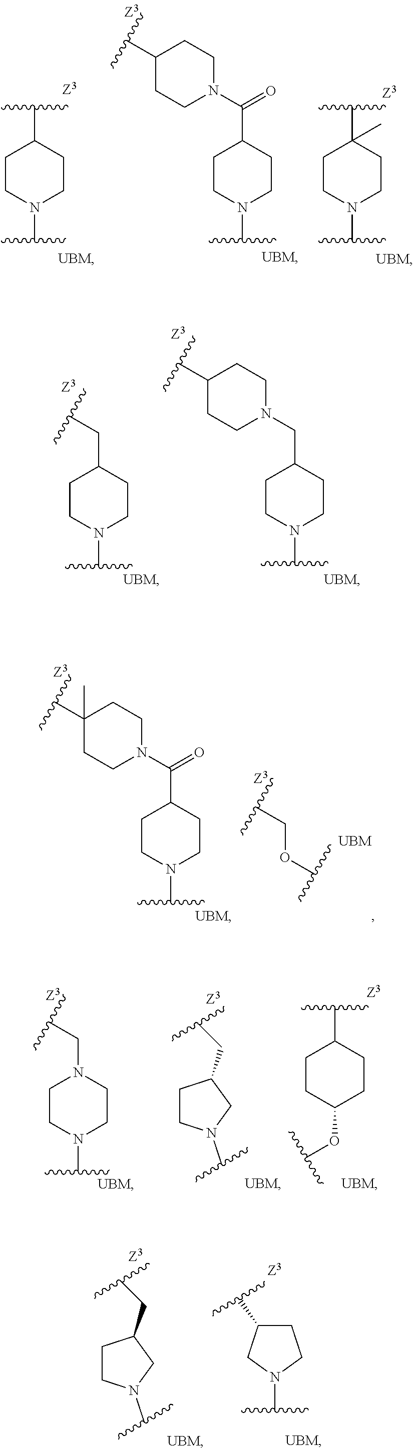 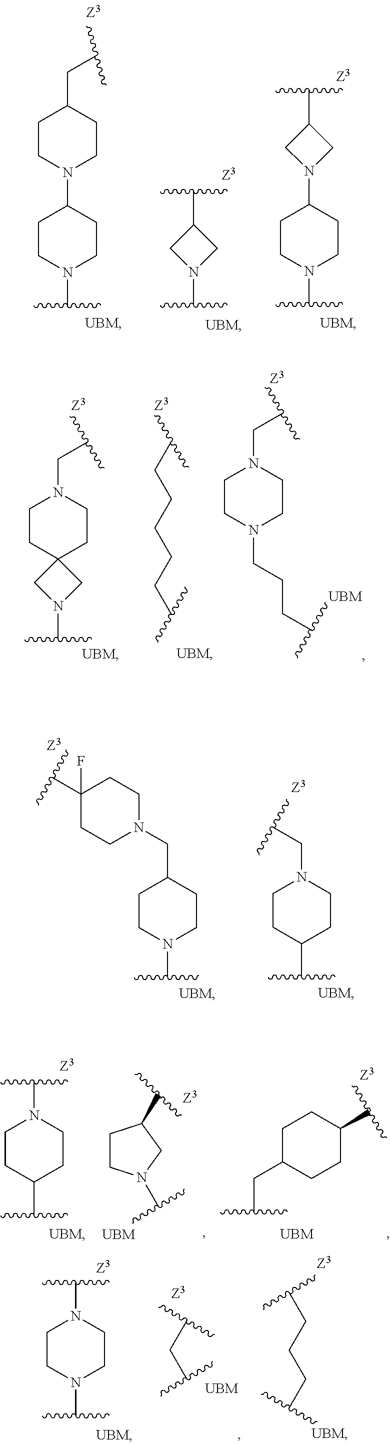 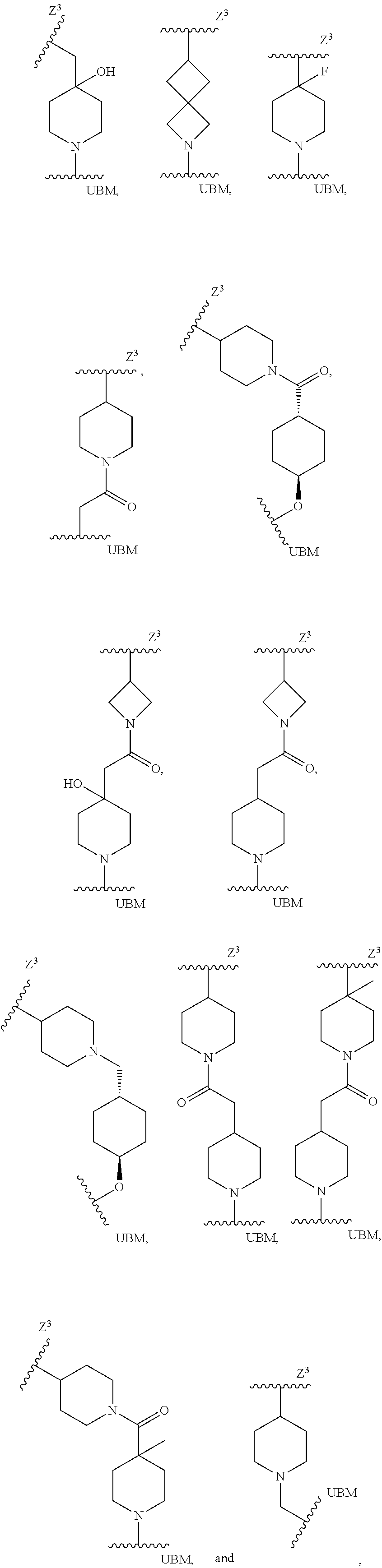
wherein 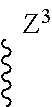
indicates attachment to Z³, and 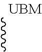
indicates attachment to UBM.

65. The compound of any one of claims 2-9, 31-33, or 53-64 selected from the group consisting of (S)—N-(3-(5-(2-aminopyrimidin-4-yl)-2-(1-(1-(1-(5-(2,6-dioxopiperidin-3-yl)pyridin-2-yl)piperidine-4-carbonyl)piperidine-4-carbonyl)piperidin-4-yl)thiazol-4-yl)-2-fluorophenyl)propane-1-sulfonamide, (S)—N-(3-(5-(2-aminopyrimidin-4-yl)-2-(1-(1-(1-(5-(2,6-dioxopiperidin-3-yl)pyridin-2-yl)-4-methylpiperidine-4-carbonyl)piperidine-4-carbonyl)piperidin-4-yl)thiazol-4-yl)-2-fluorophenyl)propane-1-sulfonamide, (S)—N-(3-(5-(2-aminopyrimidin-4-yl)-2-(1-(1-(1-(5-(2,6-dioxopiperidin-3-yl)pyridin-2-yl)piperidine-4-carbonyl)-4-methylpiperidine-4-carbonyl)piperidin-4-yl)thiazol-4-yl)-2-fluorophenyl)propane-1-sulfonamide, (R)—N-(3-(5-(2-aminopyrimidin-4-yl)-2-(1-(1-(1-(4-(2,6-dioxopiperidin-3-yl)phenyl)piperidine-4-carbonyl)-4-methylpiperidine-4-carbonyl)piperidin-4-yl)thiazol-4-yl)-2-fluorophenyl)propane-1-sulfonamide, (S)—N-(3-(2-(1-(1-(1-(4-(2,6-dioxopiperidin-3-yl)phenyl)piperidine-4-carbonyl)piperidine-4-carbonyl)piperidin-4-yl)-5-(pyrimidin-4-yl)thiazol-4-yl)-2-fluorophenyl)propane-1-sulfonamide, (S)—N-(3-(2-(1-(1-(1-(5-(2,6-dioxopiperidin-3-yl)pyridin-2-yl)piperidine-4-carbonyl)-4-methylpiperidine-4-carbonyl)piperidin-4-yl)-5-(pyrimidin-4-yl)thiazol-4-yl)-2-fluorophenyl)propane-1-sulfonamide, and (S)—N-(3-(2-(1-(1-(1-(5-(2,6-dioxopiperidin-3-yl)pyridin-2-yl)piperidine-4-carbonyl)-4-methylpiperidine-4-carbonyl)piperidin-4-yl)-5-(pyridin-4-yl)thiazol-4-yl)-2-fluorophenyl)propane-1-sulfonamide.

66. The compound of any one of claims 2, 31-33, or 53-64 selected from the group consisting of (R)—N-(3-(5-(2-aminopyrimidin-4-yl)-2-(3-(2-(4-(3-(2-(2,6-dioxopiperidin-3-yl)-1,3-dioxoisoindolin-4-yl)propyl)piperazin-1-yl)acetyl)-3-azaspiro[5.5]undecan-9-yl)thiazol-4-yl)-2-fluorophenyl)propane-1-sulfonamide, N-(3-(5-(2-aminopyrimidin-4-yl)-2-(1-((1S,4r)-4-((6-((S)-2,6-dioxopiperidin-3-yl)-3,4-dihydroisoquinolin-2(1H)-yl)methyl)cyclohexane-1-carbonyl)piperidin-4-yl)thiazol-4-yl)-2-fluorophenyl)propane-1-sulfonamide, (S)—N-(3-(2-(1-(1-((1-(5-(2,6-dioxopiperidin-3-yl)pyridin-2-yl)piperidin-4-yl)methyl)piperidine-4-carbonyl)piperidin-4-yl)-5-(pyrimidin-4-yl)thiazol-4-yl)-2-fluorophenyl)propane-1-sulfonamide, (R)—N-(3-(5-(2-aminopyrimidin-4-yl)-2-(1-(2-(4-(2-(2,6-dioxopiperidin-3-yl)-1,3-dioxoisoindolin-5-yl)piperazin-1-yl)acetyl)-4-methylpiperidin-4-yl)thiazol-4-yl)-2-fluorophenyl)propane-1-sulfonamide, (R)—N-(3-(5-(2-aminopyrimidin-4-yl)-2-(1-(2-(1′-(2-(2,6-dioxopiperidin-3-yl)-1,3-dioxoisoindolin-5-yl)-[1,4′-bipiperidin]-4-yl)acetyl)-4-methylpiperidin-4-yl)thiazol-4-yl)-2-fluorophenyl)propane-1-sulfonamide, (S)—N-(3-(5-(2-aminopyrimidin-4-yl)-2-(1-(1-((1-(5-(2,6-dioxopiperidin-3-yl)pyridin-2-yl)piperidin-4-yl)methyl)piperidine-4-carbonyl)-4-methylpiperidin-4-yl)thiazol-4-yl)-2-fluorophenyl)propane-1-sulfonamide, (S)—N-(3-(5-(2-aminopyrimidin-4-yl)-2-(1-((1-(2-(6-(2,6-dioxopiperidin-3-yl)-3,4-dihydroisoquinolin-2(1H)-yl)acetyl)piperidin-4-yl)methyl)piperidin-4-yl)thiazol-4-yl)-2-fluorophenyl)propane-1-sulfonamide, (S)—N-(3-(5-(2-aminopyrimidin-4-yl)-2-(3-((1-(4-(2,6-dioxopiperidin-3-yl)phenyl)piperidin-4-yl)methyl)-3-azaspiro[5.5]undecan-9-yl)thiazol-4-yl)-2-fluorophenyl)propane-1-sulfonamide, (S)—N-(3-(2-(1-(1-((1-(5-(2,6-dioxopiperidin-3-yl)pyridin-2-yl)piperidin-4-yl)methyl)piperidine-4-carbonyl)piperidin-4-yl)-5-(pyridin-4-yl)thiazol-4-yl)-2-fluorophenyl)propane-1-sulfonamide, (R)—N-(3-(5-(2-aminopyrimidin-4-yl)-2-(1-(2-(1′-(2-(2,6-dioxopiperidin-3-yl)-1,3-dioxoisoindolin-5-yl)-[1,4′-bipiperidin]-4-yl)acetyl)piperidin-4-yl)thiazol-4-yl)-2-fluorophenyl)propane-1-sulfonamide, (R)—N-(3-(5-(2-aminopyrimidin-4-yl)-2-(1-(2-(4-(2-(2,6-dioxopiperidin-3-yl)-1,3-dioxoisoindolin-5-yl)piperazin-1-yl)acetyl)piperidin-4-yl)thiazol-4-yl)-2-fluorophenyl)propane-1-sulfonamide, (R)—N-(3-(5-(2-aminopyrimidin-4-yl)-2-(2-(1-(1-(1-(2-(2,6-dioxopiperidin-3-yl)-1,3-dioxoisoindolin-5-yl)piperidin-4-yl)azetidine-3-carbonyl)piperidin-4-yl)propan-2-yl)thiazol-4-yl)-2-fluorophenyl)propane-1-sulfonamide, N-(3-(5-(2-aminopyrimidin-4-yl)-2-(1-((1-((1R,4r)-4-(3-((R)-2,6-dioxopiperidin-3-yl)phenoxy)cyclohexane-1-carbonyl)piperidin-4-yl)methyl)piperidin-4-yl)thiazol-4-yl)-2-fluorophenyl)propane-1-sulfonamide, (R)—N-(3-(5-(2-aminopyrimidin-4-yl)-2-(3-((1-(5-(2,6-dioxopiperidin-3-yl)pyridin-2-yl)piperidin-4-yl)methyl)-3-azaspiro[5.5]undecan-9-yl)thiazol-4-yl)-2-fluorophenyl)propane-1-sulfonamide, (S)—N-(3-(5-(2-aminopyrimidin-4-yl)-2-(1-(1-(2-(1-(4-((2,6-dioxopiperidin-3-yl)amino)-2-fluorophenyl)-4-hydroxypiperidin-4-yl)acetyl)azetidin-3-yl)piperidin-4-yl)thiazol-4-yl)-2-fluorophenyl)propane-1-sulfonamide, (S)—N-(3-(5-(2-aminopyrimidin-4-yl)-2-(4-(1-(2-(1-(5-(2,6-dioxopiperidin-3-yl)pyridin-2-yl)piperidin-4-yl)acetyl)piperidin-4-yl)phenyl)thiazol-4-yl)-2-fluorophenyl)propane-1-sulfonamide, (S)—N-(3-(5-(2-aminopyrimidin-4-yl)-2-(4-(4-((1-(4-(2,6-dioxopiperidin-3-yl)phenyl)piperidin-4-yl)methyl)piperazin-1-yl)phenyl)thiazol-4-yl)-2-fluorophenyl)propane-1-sulfonamide, (S)—N-(3-(5-(2-aminopyrimidin-4-yl)-2-(4-(1-(2-(1-(4-((2,6-dioxopiperidin-3-yl)amino)phenyl)piperidin-4-yl)acetyl)piperidin-4-yl)phenyl)thiazol-4-yl)-2-fluorophenyl)propane-1-sulfonamide, (S)—N-(3-(5-(2-aminopyrimidin-4-yl)-2-(4-(1-(1-(5-(2,6-dioxopiperidin-3-yl)pyridin-2-yl)piperidine-4-carbonyl)piperidin-4-yl)phenyl)thiazol-4-yl)-2-fluorophenyl)propane-1-sulfonamide, (S)—N-(3-(5-(2-aminopyrimidin-4-yl)-2-(4-(1-((1-(4-(2,6-dioxopiperidin-3-yl)phenyl)piperidin-4-yl)methyl)piperidin-4-yl)phenyl)thiazol-4-yl)-2-fluorophenyl)propane-1-sulfonamide, N-(3-(5-(2-aminopyrimidin-4-yl)-2-(3-(((1S,4r)-4-((5-((S)-2,6-dioxopiperidin-3-yl)pyridin-2-yl)oxy)cyclohexyl)methyl)-3-azaspiro[5.5]undecan-9-yl)thiazol-4-yl)-2-fluorophenyl)propane-1-sulfonamide, (R)—N-(3-(5-(2-aminopyrimidin-4-yl)-2-(4-(4-(1-(4-(2,6-dioxopiperidin-3-yl)phenyl)piperidine-4-carbonyl)piperazin-1-yl)phenyl)thiazol-4-yl)-2-fluorophenyl)propane-1-sulfonamide, (R)-5-(4-(9-(5-(2-aminopyrimidin-4-yl)-4-(2-fluoro-3-(propylsulfonamido)phenyl)thiazol-2-yl)-3-azaspiro[5.5]undecane-3-carbonyl)piperidin-1-yl)-N-(2,6-dioxopiperidin-3-yl)picolinamide, N-(3-(5-(2-aminopyrimidin-4-yl)-2-(3-(2-((S)-1-(5-((S)-2,6-dioxopiperidin-3-yl)pyridin-2-yl)pyrrolidin-3-yl)ethyl)-3-azaspiro[5.5]undecan-9-yl)thiazol-4-yl)-2-fluorophenyl)propane-1-sulfonamide, (S)—N-(3-(5-(2-aminopyrimidin-4-yl)-2-(1-(1-(2-(1-(4-(2,6-dioxopiperidin-3-yl)phenyl)piperidin-4-yl)acetyl)azetidin-3-yl)piperidin-4-yl)thiazol-4-yl)-2-fluorophenyl)propane-1-sulfonamide, (S)—N-(3-(5-(2-aminopyrimidin-4-yl)-2-(3-(1-(5-(2,6-dioxopiperidin-3-yl)pyridin-2-yl)piperidine-4-carbonyl)-3-azaspiro[5.5]undecan-9-yl)thiazol-4-yl)-2-fluorophenyl)propane-1-sulfonamide, (S)—N-(3-(5-(2-aminopyrimidin-4-yl)-2-(4-(1-((1-(5-(2,6-dioxopiperidin-3-yl)pyridin-2-yl)piperidin-4-yl)methyl)piperidin-4-yl)phenyl)thiazol-4-yl)-2-fluorophenyl)propane-1-sulfonamide, (S)—N-(3-(2-(1-(1-(1-(4-(2,6-dioxopiperidin-3-yl)phenyl)piperidine-4-carbonyl)piperidine-4-carbonyl)piperidin-4-yl)-5-(pyridin-4-yl)thiazol-4-yl)-2-fluorophenyl)propane-1-sulfonamide, (S)—N-(3-(2-(1-(1-((1-(5-(2,6-dioxopiperidin-3-yl)pyridin-2-yl)piperidin-4-yl)methyl)piperidine-4-carbonyl)piperidin-4-yl)-5-(2-(methylamino)pyrimidin-4-yl)thiazol-4-yl)-2-fluorophenyl)propane-1-sulfonamide, (R)—N-(3-(5-(2-aminopyrimidin-4-yl)-2-(2-(2-(1′-(2-(2,6-dioxopiperidin-3-yl)-1,3-dioxoisoindolin-5-yl)-[1,4′-bipiperidin]-4-yl)acetyl)-2-azaspiro[3.5]nonan-7-yl)thiazol-4-yl)-2-fluorophenyl)propane-1-sulfonamide, (S)—N-(3-(2-(1-(1-(1-(5-(2,6-dioxopiperidin-3-yl)pyridin-2-yl)piperidine-4-carbonyl)piperidine-4-carbonyl)piperidin-4-yl)-5-(pyridin-4-yl)thiazol-4-yl)-2-fluorophenyl)propane-1-sulfonamide, N-(3-(5-(2-aminopyrimidin-4-yl)-2-(1-((1-(((1S,4r)-4-(4-((S)-2,6-dioxopiperidin-3-yl)phenoxy)cyclohexyl)methyl)piperidin-4-yl)methyl)piperidin-4-yl)thiazol-4-yl)-2-fluorophenyl)propane-1-sulfonamide, (R)—N-(3-(5-(2-aminopyrimidin-4-yl)-2-(2-(1-(2-(2,6-dioxopiperidin-3-yl)-1,3-dioxoisoindolin-5-yl)piperidine-4-carbonyl)-2-azaspiro[3.5]nonan-7-yl)thiazol-4-yl)-2-fluorophenyl)propane-1-sulfonamide, (S)—N-(3-(2-(1-(1-(2-(1-(4-((2,6-dioxopiperidin-3-yl)amino)phenyl)piperidin-4-yl)acetyl)piperidine-4-carbonyl)piperidin-4-yl)-5-(pyrimidin-4-yl)thiazol-4-yl)-2-fluorophenyl)propane-1-sulfonamide, (S)—N-(3-(5-(2-aminopyrimidin-4-yl)-2-(2-(1-(1-(1-(5-(2,6-dioxopiperidin-3-yl)pyridin-2-yl)piperidine-4-carbonyl)piperidine-4-carbonyl)piperidin-4-yl)ethyl)thiazol-4-yl)-2-fluorophenyl)propane-1-sulfonamide, (R)—N-(3-(5-(2-aminopyrimidin-4-yl)-2-(2-(2-(4-(2-(2,6-dioxopiperidin-3-yl)-1,3-dioxoisoindolin-5-yl)piperazin-1-yl)acetyl)-2-azaspiro[3.5]nonan-7-yl)thiazol-4-yl)-2-fluorophenyl)propane-1-sulfonamide, (R)—N-(3-(5-(2-aminopyrimidin-4-yl)-2-(4-(4-(1-(5-(2,6-dioxopiperidin-3-yl)pyridin-2-yl)piperidine-4-carbonyl)piperazin-1-yl)phenyl)thiazol-4-yl)-2-fluorophenyl)propane-1-sulfonamide, (S)—N-(3-(2-(1-(1-(1-(5-(2,6-dioxopiperidin-3-yl)pyridin-2-yl)piperidine-4-carbonyl)piperidine-4-carbonyl)piperidin-4-yl)-5-(pyrimidin-4-yl)thiazol-4-yl)-2-fluorophenyl)propane-1-sulfonamide, (R)—N-(3-(5-(2-aminopyrimidin-4-yl)-2-(1-(1-(1-(4-(2,6-dioxopiperidin-3-yl)phenyl)piperidine-4-carbonyl)piperidine-4-carbonyl)piperidin-4-yl)thiazol-4-yl)-2-fluorophenyl)propane-1-sulfonamide, (R)—N-(3-(5-(2-aminopyrimidin-4-yl)-2-(4-(4-((1-(4-(2,6-dioxopiperidin-3-yl)phenyl)piperidin-4-yl)methyl)piperazin-1-yl)phenyl)thiazol-4-yl)-2-fluorophenyl)propane-1-sulfonamide, (S)—N-(3-(5-(2-aminopyrimidin-4-yl)-2-(1-((1-(1-(5-(2,6-dioxopiperidin-3-yl)pyridin-2-yl)piperidine-4-carbonyl)piperidin-4-yl)methyl)piperidin-4-yl)thiazol-4-yl)-2-fluorophenyl)propane-1-sulfonamide, (R)—N-(3-(5-(2-aminopyrimidin-4-yl)-2-(4-(4-(2-(1-(5-(2,6-dioxopiperidin-3-yl)pyridin-2-yl)piperidin-4-yl)acetyl)piperazin-1-yl)phenyl)thiazol-4-yl)-2-fluorophenyl)propane-1-sulfonamide, (R)—N-(3-(5-(2-aminopyrimidin-4-yl)-2-(3-(2-(1′-(2-(2,6-dioxopiperidin-3-yl)-1,3-dioxoisoindolin-5-yl)-[1,4′-bipiperidin]-4-yl)acetyl)-3-azaspiro[5.5]undecan-9-yl)thiazol-4-yl)-2-fluorophenyl)propane-1-sulfonamide, (S)—N-(3-(5-(2-aminopyrimidin-4-yl)-2-(1-(1-((1-(4-(2,6-dioxopiperidin-3-yl)phenyl)piperidin-4-yl)methyl)piperidine-4-carbonyl)-4-methylpiperidin-4-yl)thiazol-4-yl)-2-fluorophenyl)propane-1-sulfonamide, (S)—N-(3-(2-(1-(1-(1-(4-(2,6-dioxopiperidin-3-yl)phenyl)piperidine-4-carbonyl)-4-methylpiperidine-4-carbonyl)piperidin-4-yl)-5-(2-(isopropylamino)pyrimidin-4-yl)thiazol-4-yl)-2-fluorophenyl)propane-1-sulfonamide, (R)—N-(3-(5-(2-aminopyrimidin-4-yl)-2-(3-(2-(4-(2-(2,6-dioxopiperidin-3-yl)-1,3-dioxoisoindolin-5-yl)piperazin-1-yl)acetyl)-3-azaspiro[5.5]undecan-9-yl)thiazol-4-yl)-2-fluorophenyl)propane-1-sulfonamide, N-(3-(5-(2-aminopyrimidin-4-yl)-2-(1-((1-((1R,4r)-4-(4-((R)-2,6-dioxopiperidin-3-yl)phenoxy)cyclohexane-1-carbonyl)piperidin-4-yl)methyl)piperidin-4-yl)thiazol-4-yl)-2-fluorophenyl)propane-1-sulfonamide, (R)—N-(3-(5-(2-aminopyrimidin-4-yl)-2-(2-(6-(2-(2,6-dioxopiperidin-3-yl)-3-oxoisoindolin-5-yl)hexanoyl)-2-azaspiro[3.5]nonan-7-yl)thiazol-4-yl)-2-fluorophenyl)propane-1-sulfonamide, N-(3-(2-(1-(1-(1-(5-((S)-2,6-dioxopiperidin-3-yl)pyridin-2-yl)piperidine-4-carbonyl)piperidine-4-carbonyl)piperidin-4-yl)-5-(2-(((R)-1-hydroxypropan-2-yl)amino)pyrimidin-4-yl)thiazol-4-yl)-2-fluorophenyl)propane-1-sulfonamide, (S)—N-(3-(5-(2-aminopyrimidin-4-yl)-2-(4-(4-((1-(5-(2,6-dioxopiperidin-3-yl)pyridin-2-yl)piperidin-4-yl)methyl)piperazin-1-yl)phenyl)thiazol-4-yl)-2-fluorophenyl)propane-1-sulfonamide, rac-(3R)-3-{6-[4-(4-{4-[5-(2-aminopyrimidin-4-yl)-4-(3-{[ethyl(methyl)sulfamoyl]amino}-2-fluorophenyl)-1,3-thiazol-2-yl]piperidine-1-carbonyl}piperidine-1-carbonyl)piperidin-1-yl]pyridin-3-yl}piperidine-2,6-dione, rac-(3R)-3-{6-[4-(4-{4-[4-(3-{[ethyl(methyl)sulfamoyl]amino}-2-fluorophenyl)-5-{2-[(propan-2-yl)amino]pyrimidin-4-yl}-1,3-thiazol-2-yl]piperidine-1-carbonyl}piperidine-1-carbonyl)piperidin-1-yl]pyridin-3-yl}piperidine-2,6-dione, (S)—N-(3-(5-(2-aminopyrimidin-4-yl)-2-(1-(1-((1-(4-(2,6-dioxopiperidin-3-yl)phenyl)piperidin-4-yl)methyl)piperidine-4-carbonyl)piperidin-4-yl)thiazol-4-yl)-2-fluorophenyl)propane-1-sulfonamide, (S)—N-(3-(5-(2-aminopyrimidin-4-yl)-2-(1-(1-((1-(5-(2,6-dioxopiperidin-3-yl)pyridin-2-yl)piperidin-4-yl)methyl)piperidine-4-carbonyl)piperidin-4-yl)thiazol-4-yl)-2-fluorophenyl)propane-1-sulfonamide, (R)—N-(3-(5-(2-aminopyrimidin-4-yl)-2-(3-((1-(2-(2,6-dioxopiperidin-3-yl)-1-oxoisoindolin-5-yl)piperidin-4-yl)methyl)-3-azaspiro[5.5]undecan-9-yl)thiazol-4-yl)-2-fluorophenyl)propane-1-sulfonamide, (R)—N-(3-(5-(2-aminopyrimidin-4-yl)-2-(1-(2-(2-(2-(2,6-dioxopiperidin-3-yl)-1,3-dioxoisoindolin-5-yl)-2,7-diazaspiro[3.5]nonan-7-yl)acetyl)-4-methylpiperidin-4-yl)thiazol-4-yl)-2-fluorophenyl)propane-1-sulfonamide, (R)—N-(3-(5-(2-aminopyrimidin-4-yl)-2-(3-(1-(2-(2,6-dioxopiperidin-3-yl)-1,3-dioxoisoindolin-5-yl)piperidine-4-carbonyl)-3-azaspiro[5.5]undecan-9-yl)thiazol-4-yl)-2-fluorophenyl)propane-1-sulfonamide, (R)—N-(3-(2-(1-(2-(2-(2-(2,6-dioxopiperidin-3-yl)-1,3-dioxoisoindolin-5-yl)-2,7-diazaspiro[3.5]nonan-7-yl)acetyl)piperidin-4-yl)-5-(2-(methylamino)pyrimidin-4-yl)thiazol-4-yl)-2-fluorophenyl)propane-1-sulfonamide, (R)—N-(3-(5-(2-aminopyrimidin-4-yl)-2-(3-(6-(2-(2,6-dioxopiperidin-3-yl)-3-oxoisoindolin-5-yl)hexanoyl)-3-azaspiro[5.5]undecan-9-yl)thiazol-4-yl)-2-fluorophenyl)propane-1-sulfonamide, (R)—N-(3-(5-(2-aminopyrimidin-4-yl)-2-(1-(1-(2-(1-(5-(2,6-dioxopiperidin-3-yl)pyridin-2-yl)piperidin-4-yl)acetyl)-4-methylpiperidine-4-carbonyl)piperidin-4-yl)thiazol-4-yl)-2-fluorophenyl)propane-1-sulfonamide, (S)—N-(3-(2-(1-(1-(1-(5-(2,6-dioxopiperidin-3-yl)pyridin-2-yl)piperidine-4-carbonyl)piperidine-4-carbonyl)piperidin-4-yl)-5-(2-(methylamino)pyrimidin-4-yl)thiazol-4-yl)-2-fluorophenyl)propane-1-sulfonamide, (R)—N-(3-(5-(2-aminopyrimidin-4-yl)-2-(1-(1-(2-(1-(5-(2,6-dioxopiperidin-3-yl)pyridin-2-yl)piperidin-4-yl)acetyl)piperidine-4-carbonyl)piperidin-4-yl)thiazol-4-yl)-2-fluorophenyl)propane-1-sulfonamide, (S)—N-(3-(2-(1-(1-(1-(5-(2,6-dioxopiperidin-3-yl)pyridin-2-yl)piperidine-4-carbonyl)piperidine-4-carbonyl)piperidin-4-yl)-5-(2-(isopropylamino)pyrimidin-4-yl)thiazol-4-yl)-2-fluorophenyl)propane-1-sulfonamide, (S)—N-(3-(5-(2-(cyclopropylamino)pyrimidin-4-yl)-2-(1-(1-(1-(5-(2,6-dioxopiperidin-3-yl)pyridin-2-yl)piperidine-4-carbonyl)piperidine-4-carbonyl)piperidin-4-yl)thiazol-4-yl)-2-fluorophenyl)propane-1-sulfonamide, (S)—N-(3-(2-(1-(1-(1-(4-(2,6-dioxopiperidin-3-yl)phenyl)piperidine-4-carbonyl)-4-methylpiperidine-4-carbonyl)piperidin-4-yl)-5-(pyrimidin-4-yl)thiazol-4-yl)-2-fluorophenyl)propane-1-sulfonamide, N-(3-(5-(2-aminopyrimidin-4-yl)-2-(3-(2-((R)-1-(4-((S)-2,6-dioxopiperidin-3-yl)phenyl)pyrrolidin-3-yl)acetyl)-3-azaspiro[5.5]undecan-9-yl)thiazol-4-yl)-2-fluorophenyl)propane-1-sulfonamide, (R)—N-(3-(5-(2-aminopyrimidin-4-yl)-2-(2-(2-(2-(2-(2,6-dioxopiperidin-3-yl)-1,3-dioxoisoindolin-5-yl)-2,7-diazaspiro[3.5]nonan-7-yl)acetyl)-2-azaspiro[3.5]nonan-7-yl)thiazol-4-yl)-2-fluorophenyl)propane-1-sulfonamide, (R)—N-(3-(5-(2-aminopyrimidin-4-yl)-2-(1-((1-(2-(1-(5-(2,6-dioxopiperidin-3-yl)pyridin-2-yl)piperidin-4-yl)acetyl)piperidin-4-yl)methyl)piperidin-4-yl)thiazol-4-yl)-2-fluorophenyl)propane-1-sulfonamide, (R)—N-(3-(5-(2-aminopyrimidin-4-yl)-2-(1-(2-(2-(2-(2,6-dioxopiperidin-3-yl)-1,3-dioxoisoindolin-5-yl)-2,7-diazaspiro[3.5]nonan-7-yl)acetyl)piperidin-4-yl)thiazol-4-yl)-2-fluorophenyl)propane-1-sulfonamide, (R)—N-(3-(5-(2-aminopyrimidin-4-yl)-2-(3-(1-(1-(2,6-dioxopiperidin-3-yl)-3-methyl-2-oxo-2,3-dihydro-1H-benzo[d]imidazol-4-yl)piperidine-4-carbonyl)-3-azaspiro[5.5]undecan-9-yl)thiazol-4-yl)-2-fluorophenyl)propane-1-sulfonamide, (R)—N-(3-(2-(1-(1-((1-(4-(2,6-dioxopiperidin-3-yl)phenyl)piperidin-4-yl)methyl)piperidine-4-carbonyl)piperidin-4-yl)-5-(2-(methylamino)pyrimidin-4-yl)thiazol-4-yl)-2-fluorophenyl)propane-1-sulfonamide, (R)—N-(3-(5-(2-aminopyrimidin-4-yl)-2-(1-(1-(1-(2-(2,6-dioxopiperidin-3-yl)-1,3-dioxoisoindolin-5-yl)piperidin-4-yl)azetidine-3-carbonyl)piperidin-4-yl)thiazol-4-yl)-2-fluorophenyl)propane-1-sulfonamide, (R)—N-(3-(5-(2-aminopyrimidin-4-yl)-2-(2-(1-(1-(2-(2,6-dioxopiperidin-3-yl)-1,3-dioxoisoindolin-5-yl)piperidin-4-yl)azetidine-3-carbonyl)-2-azaspiro[3.5]nonan-7-yl)thiazol-4-yl)-2-fluorophenyl)propane-1-sulfonamide, (S)—N-(3-(5-(2-aminopyrimidin-4-yl)-2-(1-(1-((1-(1-(2,6-dioxopiperidin-3-yl)-3-methyl-2-oxo-2,3-dihydro-1H-benzo[d]imidazol-4-yl)piperidin-4-yl)methyl)piperidine-4-carbonyl)piperidin-4-yl)thiazol-4-yl)-2-fluorophenyl)propane-1-sulfonamide, (R)—N-(3-(5-(2-aminopyrimidin-4-yl)-2-(3-(2-(2-(2-(2,6-dioxopiperidin-3-yl)-1,3-dioxoisoindolin-5-yl)-2,7-diazaspiro[3.5]nonan-7-yl)acetyl)-3-azaspiro[5.5]undecan-9-yl)thiazol-4-yl)-2-fluorophenyl)propane-1-sulfonamide, (R)—N-(3-(5-(2-aminopyrimidin-4-yl)-2-(2-(1-(2-(2-(2-(2,6-dioxopiperidin-3-yl)-1,3-dioxoisoindolin-5-yl)-2,7-diazaspiro[3.5]nonan-7-yl)acetyl)piperidin-4-yl)propan-2-yl)thiazol-4-yl)-2-fluorophenyl)propane-1-sulfonamide, and (R)-4-((4-(5-(2-aminopyrimidin-4-yl)-4-(2-fluoro-3-(propylsulfonamido)phenyl)thiazol-2-yl)piperidin-1-yl)methyl)-N-(2,6-dioxopiperidin-3-yl)benzamide.

67. The compound of any one of claims 2, 18-24, 31-33, or 53-64 selected from the group consisting of (R)-4-(4-((6-((4-(2-(tert-butyl)-4-(2-fluoro-3-(propylsulfonamido)phenyl)thiazol-5-yl)pyrimidin-2-yl)amino)-2-azaspiro[3.3]heptan-2-yl)methyl)piperidin-1-yl)-N-(2,6-dioxopiperidin-3-yl)benzamide, N-(3-(2-(tert-butyl)-5-(2-((2-(2-((S)-1-(5-((S)-2,6-dioxopiperidin-3-yl)pyridin-2-yl)pyrrolidin-3-yl)ethyl)-2-azaspiro[3.3]heptan-6-yl)amino)pyrimidin-4-yl)thiazol-4-yl)-2-fluorophenyl)propane-1-sulfonamide, (R)—N-(3-(2-(tert-butyl)-5-(2-((2-(2-(1′-(2-(2,6-dioxopiperidin-3-yl)-1,3-dioxoisoindolin-5-yl)-[1,4′-bipiperidin]-4-yl)acetyl)-2-azaspiro[3.3]heptan-6-yl)amino)pyrimidin-4-yl)thiazol-4-yl)-2-fluorophenyl)propane-1-sulfonamide, (R)—N-(3-(2-(tert-butyl)-5-(2-((2-(1-(1-(2,6-dioxopiperidin-3-yl)-3-methyl-2-oxo-2,3-dihydro-1H-benzo[d]imidazol-4-yl)piperidine-4-carbonyl)-2-azaspiro[3.3]heptan-6-yl)amino)pyrimidin-4-yl)thiazol-4-yl)-2-fluorophenyl)propane-1-sulfonamide, (R)—N-(3-(2-(tert-butyl)-5-(2-((2-(1-(1-(2-(2,6-dioxopiperidin-3-yl)-1,3-dioxoisoindolin-5-yl)piperidin-4-yl)azetidine-3-carbonyl)-2-azaspiro[3.3]heptan-6-yl)amino)pyrimidin-4-yl)thiazol-4-yl)-2-fluorophenyl)propane-1-sulfonamide, (R)—N-(3-(2-(tert-butyl)-5-(2-((2-(2-(2-(2-(2,6-dioxopiperidin-3-yl)-1,3-dioxoisoindolin-5-yl)-2,7-diazaspiro[3.5]nonan-7-yl)acetyl)-2-azaspiro[3.3]heptan-6-yl)amino)pyrimidin-4-yl)thiazol-4-yl)-2-fluorophenyl)propane-1-sulfonamide, (R)—N-(3-(2-(tert-butyl)-5-(2-((2-(6-(2-(2,6-dioxopiperidin-3-yl)-3-oxoisoindolin-5-yl)hexanoyl)-2-azaspiro[3.3]heptan-6-yl)amino)pyrimidin-4-yl)thiazol-4-yl)-2-fluorophenyl)propane-1-sulfonamide, (R)—N-(3-(2-(tert-butyl)-5-(2-((2-((1-((1-(4-(2,6-dioxopiperidin-3-yl)phenyl)piperidin-4-yl)methyl)piperidin-4-yl)methyl)-2-azaspiro[3.3]heptan-6-yl)amino)pyrimidin-4-yl)thiazol-4-yl)-2-fluorophenyl)propane-1-sulfonamide, (R)—N-(3-(2-(tert-butyl)-5-(2-((2-(2-(4-(3-(2-(2,6-dioxopiperidin-3-yl)-1,3-dioxoisoindolin-4-yl)propyl)piperazin-1-yl)acetyl)-2-azaspiro[3.3]heptan-6-yl)amino)pyrimidin-4-yl)thiazol-4-yl)-2-fluorophenyl)propane-1-sulfonamide, (R)—N-(3-(2-(tert-butyl)-5-(2-((2-(1-((1-(4-(2,6-dioxopiperidin-3-yl)phenyl)piperidin-4-yl)methyl)-4-fluoropiperidine-4-carbonyl)-2-azaspiro[3.3]heptan-6-yl)amino)pyrimidin-4-yl)thiazol-4-yl)-2-fluorophenyl)propane-1-sulfonamide, (R)—N-(3-(2-(tert-butyl)-5-(2-((2-((1-(1-(4-(2,6-dioxopiperidin-3-yl)phenyl)piperidine-4-carbonyl)piperidin-4-yl)methyl)-2-azaspiro[3.3]heptan-6-yl)amino)pyrimidin-4-yl)thiazol-4-yl)-2-fluorophenyl)propane-1-sulfonamide, (S)—N-(3-(2-(tert-butyl)-5-(2-((1′-((1-(5-(2,6-dioxopiperidin-3-yl)pyridin-2-yl)piperidin-4-yl)methyl)-[1,4′-bipiperidin]-4-yl)amino)pyrimidin-4-yl)thiazol-4-yl)-2-fluorophenyl)propane-1-sulfonamide, (R)—N-(3-(2-(tert-butyl)-5-(2-((7-(2-(4-(4-((2,6-dioxopiperidin-3-yl)amino)phenyl)piperidin-1-yl)acetyl)-7-azaspiro[3.5]nonan-2-yl)amino)pyrimidin-4-yl)thiazol-4-yl)-2-fluorophenyl)propane-1-sulfonamide, N-(3-(2-(tert-butyl)-5-(2-((7-(((1S,4r)-4-(4-((S)-2,6-dioxopiperidin-3-yl)phenoxy)cyclohexyl)methyl)-7-azaspiro[3.5]nonan-2-yl)amino)pyrimidin-4-yl)thiazol-4-yl)-2-fluorophenyl)propane-1-sulfonamide, (R)—N-(3-(2-(tert-butyl)-5-(2-((7-((1-(2-(2,6-dioxopiperidin-3-yl)-1-oxo-1,2-dihydroisoquinolin-6-yl)piperidin-4-yl)methyl)-7-azaspiro[3.5]nonan-2-yl)amino)pyrimidin-4-yl)thiazol-4-yl)-2-fluorophenyl)propane-1-sulfonamide, (R)—N-(3-(2-(tert-butyl)-5-(2-((7-((1-(4-(2,6-dioxopiperidin-3-yl)phenyl)piperidin-4-yl)methyl)-7-azaspiro[3.5]nonan-2-yl)amino)pyrimidin-4-yl)thiazol-4-yl)-2-fluorophenyl)propane-1-sulfonamide, and (R)—N-(3-(2-(tert-butyl)-5-(2-((7-(2-(4-(2-(2,6-dioxopiperidin-3-yl)-1,3-dioxoisoindolin-5-yl)piperazin-1-yl)acetyl)-7-azaspiro[3.5]nonan-2-yl)amino)pyrimidin-4-yl)thiazol-4-yl)-2-fluorophenyl)propane-1-sulfonamide.

68. The compound of any one of claims 2, 31-33, or 53-64 selected from the group consisting of (S)—N-(3-(2-(tert-butyl)-5-(2-((4-(1-((1-(5-(2,6-dioxopiperidin-3-yl)pyridin-3-yl)piperidin-4-yl)methyl)piperidin-4-yl)phenyl)amino)pyrimidin-4-yl)thiazol-4-yl)-2-fluorophenyl)propane-1-sulfonamide, (R)—N-(3-(2-(tert-butyl)-5-(2-((4-(1-(2-((2-(2,6-dioxopiperidin-3-yl)-1,3-dioxoisoindolin-5-yl)oxy)acetyl)piperidin-4-yl)phenyl)amino)pyrimidin-4-yl)thiazol-4-yl)-2-fluorophenyl)propane-1-sulfonamide, (R)—N-(3-(2-(tert-butyl)-5-(2-((4-(1-(2-((2-(2,6-dioxopiperidin-3-yl)-1,3-dioxoisoindolin-5-yl)oxy)acetyl)piperidin-4-yl)phenyl)amino)pyrimidin-4-yl)thiazol-4-yl)-2-fluorophenyl)propane-1-sulfonamide, (R)—N-(3-(2-(tert-butyl)-5-(2-((4-(1-(2-(4-(4-(2,6-dioxopiperidin-3-yl)pyridin-2-yl)piperazin-1-yl)acetyl)piperidin-4-yl)phenyl)amino)pyrimidin-4-yl)thiazol-4-yl)-2-fluorophenyl)propane-1-sulfonamide, N-(3-(2-(tert-butyl)-5-(2-((4-(1-(2-((S)-1-(5-((S)-2,6-dioxopiperidin-3-yl)pyridin-2-yl)pyrrolidin-3-yl)ethyl)piperidin-4-yl)phenyl)amino)pyrimidin-4-yl)thiazol-4-yl)-2-fluorophenyl)propane-1-sulfonamide, (R)-4-(4-((4-(4-((4-(2-(tert-butyl)-4-(2-fluoro-3-(propylsulfonamido)phenyl)thiazol-5-yl)pyrimidin-2-yl)amino)phenyl)piperidin-1-yl)methyl)piperidin-1-yl)-N-(2,6-dioxopiperidin-3-yl)benzamide, (R)-5-(4-((4-(4-((4-(2-(tert-butyl)-4-(2-fluoro-3-(propylsulfonamido)phenyl)thiazol-5-yl)pyrimidin-2-yl)amino)phenyl)piperidin-1-yl)methyl)piperidin-1-yl)-N-(2,6-dioxopiperidin-3-yl)picolinamide, N-(3-(2-(tert-butyl)-5-(2-((4-(1-(((1S,4r)-4-((5-((S)-2,6-dioxopiperidin-3-yl)pyridin-2-yl)oxy)cyclohexyl)methyl)piperidin-4-yl)phenyl)amino)pyrimidin-4-yl)thiazol-4-yl)-2-fluorophenyl)propane-1-sulfonamide, (S)—N-(3-(2-(tert-butyl)-5-(2-((4-(1-((1-(4-(2,6-dioxopiperidin-3-yl)phenyl)piperidin-4-yl)methyl)piperidin-4-yl)phenyl)amino)pyrimidin-4-yl)thiazol-4-yl)-2-fluorophenyl)propane-1-sulfonamide, N-(3-(2-(tert-butyl)-5-(2-((4-(1-(2-((S)-1-(4-((S)-2,6-dioxopiperidin-3-yl)phenyl)pyrrolidin-3-yl)acetyl)piperidin-4-yl)phenyl)amino)pyrimidin-4-yl)thiazol-4-yl)-2-fluorophenyl)propane-1-sulfonamide, N-(3-(2-(tert-butyl)-5-(2-((4-(1-(2-((R)-1-(4-((S)-2,6-dioxopiperidin-3-yl)phenyl)pyrrolidin-3-yl)acetyl)piperidin-4-yl)phenyl)amino)pyrimidin-4-yl)thiazol-4-yl)-2-fluorophenyl)propane-1-sulfonamide, N-(3-(2-(tert-butyl)-5-(2-((4-(1-((R)-1-(5-((S)-2,6-dioxopiperidin-3-yl)pyridin-2-yl)pyrrolidine-3-carbonyl)piperidin-4-yl)phenyl)amino)pyrimidin-4-yl)thiazol-4-yl)-2-fluorophenyl)propane-1-sulfonamide, (S)—N-(3-(2-(tert-butyl)-5-(2-((4-(1-(1-(4-(2,6-dioxopiperidin-3-yl)pyridin-2-yl)piperidine-4-carbonyl)piperidin-4-yl)phenyl)amino)pyrimidin-4-yl)thiazol-4-yl)-2-fluorophenyl)propane-1-sulfonamide, (R)—N-(3-(2-(tert-butyl)-5-(2-((4-(1-(2-(1′-(2-(2,6-dioxopiperidin-3-yl)-1,3-dioxoisoindolin-5-yl)-[1,4′-bipiperidin]-4-yl)acetyl)piperidin-4-yl)phenyl)amino)pyrimidin-4-yl)thiazol-4-yl)-2-fluorophenyl)propane-1-sulfonamide, (S)—N-(3-(2-(tert-butyl)-5-(2-((4-(1-(1-(5-(2,6-dioxopiperidin-3-yl)pyridin-2-yl)piperidine-4-carbonyl)piperidin-4-yl)phenyl)amino)pyrimidin-4-yl)thiazol-4-yl)-2-fluorophenyl)propane-1-sulfonamide, N-(3-(2-(tert-butyl)-5-(2-((4-(1-(2-((S)-1-(1-((R)-2,6-dioxopiperidin-3-yl)-3-methyl-2-oxo-2,3-dihydro-1H-benzo[d]imidazol-5-yl)pyrrolidin-3-yl)acetyl)piperidin-4-yl)phenyl)amino)pyrimidin-4-yl)thiazol-4-yl)-2-fluorophenyl)propane-1-sulfonamide, (R)—N-(3-(2-(tert-butyl)-5-(2-((4-(1-(1-(1-(2,6-dioxopiperidin-3-yl)-3-methyl-2-oxo-2,3-dihydro-1H-benzo[d]imidazol-4-yl)piperidine-4-carbonyl)piperidin-4-yl)phenyl)amino)pyrimidin-4-yl)thiazol-4-yl)-2-fluorophenyl)propane-1-sulfonamide, (S)—N-(3-(2-(tert-butyl)-5-(2-((4-(1-(1-(5-(2,6-dioxopiperidin-3-yl)pyridin-2-yl)azetidine-3-carbonyl)piperidin-4-yl)phenyl)amino)pyrimidin-4-yl)thiazol-4-yl)-2-fluorophenyl)propane-1-sulfonamide, (R)—N-(3-(2-(tert-butyl)-5-(2-((4-(1-(1-(2-(2,6-dioxopiperidin-3-yl)-1,3-dioxoisoindolin-5-yl)piperidine-4-carbonyl)piperidin-4-yl)phenyl)amino)pyrimidin-4-yl)thiazol-4-yl)-2-fluorophenyl)propane-1-sulfonamide, (R)—N-(3-(2-(tert-butyl)-5-(2-((4-(1-(2-(2-(2-(2,6-dioxopiperidin-3-yl)-1,3-dioxoisoindolin-5-yl)-2,7-diazaspiro[3.5]nonan-7-yl)acetyl)piperidin-4-yl)phenyl)amino)pyrimidin-4-yl)thiazol-4-yl)-2-fluorophenyl)propane-1-sulfonamide, (R)—N-(3-(2-(tert-butyl)-5-(2-((4-(1-(6-(2-(2,6-dioxopiperidin-3-yl)-3-oxoisoindolin-5-yl)hexanoyl)piperidin-4-yl)phenyl)amino)pyrimidin-4-yl)thiazol-4-yl)-2-fluorophenyl)propane-1-sulfonamide, (R)—N-(3-(2-(tert-butyl)-5-(2-((4-(1-(2-(4-(2-(2,6-dioxopiperidin-3-yl)-1,3-dioxoisoindolin-5-yl)piperazin-1-yl)acetyl)piperidin-4-yl)phenyl)amino)pyrimidin-4-yl)thiazol-4-yl)-2-fluorophenyl)propane-1-sulfonamide, (S)-5-(4-((4-(((4-(2-(tert-butyl)-4-(2-fluoro-3-(propylsulfonamido)phenyl)thiazol-5-yl)pyrimidin-2-yl)amino)methyl)piperidin-1-yl)methyl)piperidin-1-yl)-N-(2,6-dioxopiperidin-3-yl)picolinamide, (S)-5-(4-((4-(1-((4-(2-(tert-butyl)-4-(2-fluoro-3-(propylsulfonamido)phenyl)thiazol-5-yl)pyrimidin-2-yl)amino)-2-methylpropan-2-yl)piperidin-1-yl)methyl)piperidin-1-yl)-N-(2,6-dioxopiperidin-3-yl)picolinamide, (R)—N-(3-(2-(tert-butyl)-5-(2-(((1-((1-(4-(2,6-dioxopiperidin-3-yl)phenyl)piperidin-4-yl)methyl)piperidin-4-yl)methyl)amino)pyrimidin-4-yl)thiazol-4-yl)-2-fluorophenyl)propane-1-sulfonamide, (R)—N-(3-(2-(tert-butyl)-5-(2-((2-(1-((1-(4-(2,6-dioxopiperidin-3-yl)phenyl)piperidin-4-yl)methyl)piperidin-4-yl)-2-methylpropyl)amino)pyrimidin-4-yl)thiazol-4-yl)-2-fluorophenyl)propane-1-sulfonamide, 5-(4-(((R)-3-(((4-(2-(tert-butyl)-4-(2-fluoro-3-(propylsulfonamido)phenyl)thiazol-5-yl)pyrimidin-2-yl)amino)methyl)pyrrolidin-1-yl)methyl)piperidin-1-yl)-N—((S)-2,6-dioxopiperidin-3-yl)picolinamide, N-(3-(2-(tert-butyl)-5-(2-(((1r,4r)-4-((4-((1-(4-((S)-2,6-dioxopiperidin-3-yl)phenyl)piperidin-4-yl)methyl)piperazin-1-yl)methyl)cyclohexyl)amino)pyrimidin-4-yl)thiazol-4-yl)-2-fluorophenyl)propane-1-sulfonamide, N-(3-(2-(tert-butyl)-5-(2-(((1s,4s)-4-(4-(1-(5-((S)-2,6-dioxopiperidin-3-yl)pyridin-2-yl)piperidine-4-carbonyl)piperazin-1-yl)cyclohexyl)amino)pyrimidin-4-yl)thiazol-4-yl)-2-fluorophenyl)propane-1-sulfonamide, N-(3-(2-(tert-butyl)-5-(2-(((1r,4r)-4-(4-(1-(5-((S)-2,6-dioxopiperidin-3-yl)pyridin-2-yl)piperidine-4-carbonyl)piperazin-1-yl)cyclohexyl)amino)pyrimidin-4-yl)thiazol-4-yl)-2-fluorophenyl)propane-1-sulfonamide, N-(3-(2-(tert-butyl)-5-(2-(((1r,4r)-4-(4-((1-(5-((S)-2,6-dioxopiperidin-3-yl)pyridin-2-yl)piperidin-4-yl)methyl)piperazin-1-yl)cyclohexyl)amino)pyrimidin-4-yl)thiazol-4-yl)-2-fluorophenyl)propane-1-sulfonamide, N-(3-(2-(tert-butyl)-5-(2-(((1s,4s)-4-(4-((1-(5-((S)-2,6-dioxopiperidin-3-yl)pyridin-2-yl)piperidin-4-yl)methyl)piperazin-1-yl)cyclohexyl)amino)pyrimidin-4-yl)thiazol-4-yl)-2-fluorophenyl)propane-1-sulfonamide, (3R)-3-{4-[4-({4-[6-({4-[2-tert-butyl-4-(3-{[ethyl(methyl)sulfamoyl]amino}-2-fluorophenyl)-1,3-thiazol-5-yl]pyrimidin-2-yl}amino)pyridin-3-yl]piperazin-1-yl}methyl)piperidin-1-yl]phenyl}piperidine-2,6-dione, (3S)-3-{4-[4-({4-[6-({4-[2-tert-butyl-4-(3-{[ethyl(methyl)sulfamoyl]amino}-2-fluorophenyl)-1,3-thiazol-5-yl]pyrimidin-2-yl}amino)pyridin-3-yl]piperazin-1-yl}methyl)piperidin-1-yl]phenyl}piperidine-2,6-dione, rac-(3S)-3-{6-[4-({4-[6-({4-[2-tert-butyl-4-(3-{[ethyl(methyl)sulfamoyl]amino}-2-fluorophenyl)-1,3-thiazol-5-yl]pyrimidin-2-yl}amino)pyridin-3-yl]piperazin-1-yl}methyl)piperidin-1-yl]pyridin-3-yl}piperidine-2,6-dione, rac-(3S)-3-(2-{[(1r,4r)-4-{4-[6-({4-[2-tert-butyl-4-(3-{[ethyl(methyl)sulfamoyl]amino}-2-fluorophenyl)-1,3-thiazol-5-yl]pyrimidin-2-yl}amino)pyridin-3-yl]piperazine-1-carbonyl}cyclohexyl]methyl}-1,2,3,4-tetrahydroisoquinolin-6-yl)piperidine-2,6-dione, rac-(3S)-3-(2-{[(1r,4r)-4-{4-[6-({4-[2-tert-butyl-4-(3-{[ethyl(methyl)sulfamoyl]amino}-2-fluorophenyl)-1,3-thiazol-5-yl]pyrimidin-2-yl}amino)pyridin-3-yl]piperazine-1-carbonyl}cyclohexyl]methyl}-1,2,3,4-tetrahydroisoquinolin-7-yl)piperidine-2,6-dione, (R)—N-(3-(2-(tert-butyl)-5-(2-((2-(2-(4-(4-((2,6-dioxopiperidin-3-yl)amino)phenyl)piperidin-1-yl)acetyl)-1,2,3,4-tetrahydroisoquinolin-6-yl)amino)pyrimidin-4-yl)thiazol-4-yl)-2-fluorophenyl)propane-1-sulfonamide, (R)-5-(4-(6-((4-(2-(tert-butyl)-4-(2-fluoro-3-(propylsulfonamido)phenyl)thiazol-5-yl)pyrimidin-2-yl)amino)-1,2,3,4-tetrahydroisoquinoline-2-carbonyl)piperidin-1-yl)-N-(2,6-dioxopiperidin-3-yl)picolinamide, N-(3-(2-(tert-butyl)-5-(2-((2-((R)-1-(5-((S)-2,6-dioxopiperidin-3-yl)pyridin-2-yl)pyrrolidine-3-carbonyl)-1,2,3,4-tetrahydroisoquinolin-6-yl)amino)pyrimidin-4-yl)thiazol-4-yl)-2-fluorophenyl)propane-1-sulfonamide, (S)—N-(3-(2-(tert-butyl)-5-(2-((2-(2-(4-(4-(2,6-dioxopiperidin-3-yl)pyridin-2-yl)piperazin-1-yl)acetyl)-1,2,3,4-tetrahydroisoquinolin-6-yl)amino)pyrimidin-4-yl)thiazol-4-yl)-2-fluorophenyl)propane-1-sulfonamide, (S)—N-(3-(2-(tert-butyl)-5-(2-((1-((1-(2-(6-(2,6-dioxopiperidin-3-yl)-3,4-dihydroisoquinolin-2(1H)-yl)acetyl)piperidin-4-yl)methyl)-1H-pyrazol-4-yl)amino)pyrimidin-4-yl)thiazol-4-yl)-2-fluorophenyl)propane-1-sulfonamide, N-(3-(2-(tert-butyl)-5-(2-((1-((1-((1S,4r)-4-(4-((S)-2,6-dioxopiperidin-3-yl)phenoxy)cyclohexane-1-carbonyl)piperidin-4-yl)methyl)-1H-pyrazol-4-yl)amino)pyrimidin-4-yl)thiazol-4-yl)-2-fluorophenyl)propane-1-sulfonamide, N-(3-(2-(tert-butyl)-5-(2-((1-((1-((1S,4r)-4-((5-((S)-2,6-dioxopiperidin-3-yl)pyridin-2-yl)oxy)cyclohexane-1-carbonyl)piperidin-4-yl)methyl)-1H-pyrazol-4-yl)amino)pyrimidin-4-yl)thiazol-4-yl)-2-fluorophenyl)propane-1-sulfonamide, (R)—N-(3-(2-(tert-butyl)-5-(2-((1-((1-(2-(4-(4-((2,6-dioxopiperidin-3-yl)amino)phenyl)piperidin-1-yl)acetyl)piperidin-4-yl)methyl)-1H-pyrazol-4-yl)amino)pyrimidin-4-yl)thiazol-4-yl)-2-fluorophenyl)propane-1-sulfonamide, (R)-5-(4-(4-((4-((4-(2-(tert-butyl)-4-(2-fluoro-3-(propylsulfonamido)phenyl)thiazol-5-yl)pyrimidin-2-yl)amino)-1H-pyrazol-1-yl)methyl)piperidine-1-carbonyl)piperidin-1-yl)-N-(2,6-dioxopiperidin-3-yl)picolinamide, N-(3-(2-(tert-butyl)-5-(2-((1-((1-((R)-1-(5-((S)-2,6-dioxopiperidin-3-yl)pyridin-2-yl)pyrrolidine-3-carbonyl)piperidin-4-yl)methyl)-1H-pyrazol-4-yl)amino)pyrimidin-4-yl)thiazol-4-yl)-2-fluorophenyl)propane-1-sulfonamide, (S)—N-(3-(2-(tert-butyl)-5-(2-((1-((1-(2-(4-(4-(2,6-dioxopiperidin-3-yl)pyridin-2-yl)piperazin-1-yl)acetyl)piperidin-4-yl)methyl)-1H-pyrazol-4-yl)amino)pyrimidin-4-yl)thiazol-4-yl)-2-fluorophenyl)propane-1-sulfonamide, N-(3-(2-(tert-butyl)-5-(2-((1-((1-(2-((S)-1-(4-((S)-2,6-dioxopiperidin-3-yl)phenyl)pyrrolidin-3-yl)acetyl)piperidin-4-yl)methyl)-1H-pyrazol-4-yl)amino)pyrimidin-4-yl)thiazol-4-yl)-2-fluorophenyl)propane-1-sulfonamide, N-(3-(2-(tert-butyl)-5-(2-((1-((1-(2-((R)-1-(4-((S)-2,6-dioxopiperidin-3-yl)phenyl)pyrrolidin-3-yl)acetyl)piperidin-4-yl)methyl)-1H-pyrazol-4-yl)amino)pyrimidin-4-yl)thiazol-4-yl)-2-fluorophenyl)propane-1-sulfonamide, N-(3-(2-(tert-butyl)-5-(2-((1-(1-((1S,4r)-4-(4-((S)-2,6-dioxopiperidin-3-yl)phenoxy)cyclohexane-1-carbonyl)piperidin-4-yl)-1H-pyrazol-4-yl)amino)pyrimidin-4-yl)thiazol-4-yl)-2-fluorophenyl)propane-1-sulfonamide, N-(3-(2-(tert-butyl)-5-(2-((1-(1-((1S,4r)-4-((5-((S)-2,6-dioxopiperidin-3-yl)pyridin-2-yl)oxy)cyclohexane-1-carbonyl)piperidin-4-yl)-1H-pyrazol-4-yl)amino)pyrimidin-4-yl)thiazol-4-yl)-2-fluorophenyl)propane-1-sulfonamide, (R)—N-(3-(2-(tert-butyl)-5-(2-((1-(1-(2-(4-(4-((2,6-dioxopiperidin-3-yl)amino)phenyl)piperidin-1-yl)acetyl)piperidin-4-yl)-1H-pyrazol-4-yl)amino)pyrimidin-4-yl)thiazol-4-yl)-2-fluorophenyl)propane-1-sulfonamide, (R)-5-(4-(4-(4-((4-(2-(tert-butyl)-4-(2-fluoro-3-(propylsulfonamido)phenyl)thiazol-5-yl)pyrimidin-2-yl)amino)-1H-pyrazol-1-yl)piperidine-1-carbonyl)piperidin-1-yl)-N-(2,6-dioxopiperidin-3-yl)picolinamide, N-(3-(2-(tert-butyl)-5-(2-((1-(1-((R)-1-(5-((S)-2,6-dioxopiperidin-3-yl)pyridin-2-yl)pyrrolidine-3-carbonyl)piperidin-4-yl)-1H-pyrazol-4-yl)amino)pyrimidin-4-yl)thiazol-4-yl)-2-fluorophenyl)propane-1-sulfonamide, N-(3-(2-(tert-butyl)-5-(2-((1-(1-(2-((S)-1-(4-((S)-2,6-dioxopiperidin-3-yl)phenyl)pyrrolidin-3-yl)acetyl)piperidin-4-yl)-1H-pyrazol-4-yl)amino)pyrimidin-4-yl)thiazol-4-yl)-2-fluorophenyl)propane-1-sulfonamide, (S)—N-(3-(2-(tert-butyl)-5-(2-((3-(1-(2-(6-(2,6-dioxopiperidin-3-yl)-3,4-dihydroisoquinolin-2(1H)-yl)acetyl)piperidin-4-yl)phenyl)amino)pyrimidin-4-yl)thiazol-4-yl)-2-fluorophenyl)propane-1-sulfonamide, (R)—N-(3-(2-(tert-butyl)-5-(2-((3-(1-(2-(4-(4-((2,6-dioxopiperidin-3-yl)amino)phenyl)piperidin-1-yl)acetyl)piperidin-4-yl)phenyl)amino)pyrimidin-4-yl)thiazol-4-yl)-2-fluorophenyl)propane-1-sulfonamide, N-(3-(2-(tert-butyl)-5-(2-((3-(1-((R)-1-(5-((S)-2,6-dioxopiperidin-3-yl)pyridin-2-yl)pyrrolidine-3-carbonyl)piperidin-4-yl)phenyl)amino)pyrimidin-4-yl)thiazol-4-yl)-2-fluorophenyl)propane-1-sulfonamide, (S)—N-(3-(2-(tert-butyl)-5-(2-((3-((1-(2-(6-(2,6-dioxopiperidin-3-yl)-3,4-dihydroisoquinolin-2(1H)-yl)acetyl)piperidin-4-yl)oxy)phenyl)amino)pyrimidin-4-yl)thiazol-4-yl)-2-fluorophenyl)propane-1-sulfonamide, (S)—N-(3-(2-(tert-butyl)-5-(2-((3-((1-(1-(4-(2,6-dioxopiperidin-3-yl)pyridin-2-yl)piperidine-4-carbonyl)piperidin-4-yl)oxy)phenyl)amino)pyrimidin-4-yl)thiazol-4-yl)-2-fluorophenyl)propane-1-sulfonamide, (S)—N-(3-(2-(tert-butyl)-5-(2-((3-((1-(1-(3-(2,6-dioxopiperidin-3-yl)phenyl)piperidine-4-carbonyl)piperidin-4-yl)oxy)phenyl)amino)pyrimidin-4-yl)thiazol-4-yl)-2-fluorophenyl)propane-1-sulfonamide, N-(3-(2-(tert-butyl)-5-(2-((3-((1-((1S,4r)-4-(4-((S)-2,6-dioxopiperidin-3-yl)phenoxy)cyclohexane-1-carbonyl)piperidin-4-yl)oxy)phenyl)amino)pyrimidin-4-yl)thiazol-4-yl)-2-fluorophenyl)propane-1-sulfonamide, N-(3-(2-(tert-butyl)-5-(2-((3-((1-((1S,4r)-4-((5-((S)-2,6-dioxopiperidin-3-yl)pyridin-2-yl)oxy)cyclohexane-1-carbonyl)piperidin-4-yl)oxy)phenyl)amino)pyrimidin-4-yl)thiazol-4-yl)-2-fluorophenyl)propane-1-sulfonamide, (R)—N-(3-(2-(tert-butyl)-5-(2-((3-((1-(2-(4-(4-((2,6-dioxopiperidin-3-yl)amino)phenyl)piperidin-1-yl)acetyl)piperidin-4-yl)oxy)phenyl)amino)pyrimidin-4-yl)thiazol-4-yl)-2-fluorophenyl)propane-1-sulfonamide, (R)-5-(4-(4-(3-((4-(2-(tert-butyl)-4-(2-fluoro-3-(propylsulfonamido)phenyl)thiazol-5-yl)pyrimidin-2-yl)amino)phenoxy)piperidine-1-carbonyl)piperidin-1-yl)-N-(2,6-dioxopiperidin-3-yl)picolinamide, N-(3-(2-(tert-butyl)-5-(2-((3-((1-((R)-1-(5-((S)-2,6-dioxopiperidin-3-yl)pyridin-2-yl)pyrrolidine-3-carbonyl)piperidin-4-yl)oxy)phenyl)amino)pyrimidin-4-yl)thiazol-4-yl)-2-fluorophenyl)propane-1-sulfonamide, (S)—N-(3-(2-(tert-butyl)-5-(2-((3-((1-(2-(4-(4-(2,6-dioxopiperidin-3-yl)pyridin-2-yl)piperazin-1-yl)acetyl)piperidin-4-yl)oxy)phenyl)amino)pyrimidin-4-yl)thiazol-4-yl)-2-fluorophenyl)propane-1-sulfonamide, N-(3-(2-(tert-butyl)-5-(2-((3-((1-(2-((S)-1-(4-((S)-2,6-dioxopiperidin-3-yl)phenyl)pyrrolidin-3-yl)acetyl)piperidin-4-yl)oxy)phenyl)amino)pyrimidin-4-yl)thiazol-4-yl)-2-fluorophenyl)propane-1-sulfonamide, N-(3-(2-(tert-butyl)-5-(2-((3-((1-(2-((R)-1-(4-((S)-2,6-dioxopiperidin-3-yl)phenyl)pyrrolidin-3-yl)acetyl)piperidin-4-yl)oxy)phenyl)amino)pyrimidin-4-yl)thiazol-4-yl)-2-fluorophenyl)propane-1-sulfonamide, (S)—N-(3-(2-(tert-butyl)-5-(2-((4-(1-((1-(4-(2,6-dioxopiperidin-3-yl)phenyl)piperidin-4-yl)methyl)piperidin-4-yl)phenyl)amino)pyrimidin-4-yl)thiazol-4-yl)-2-fluorophenyl)propane-1-sulfonamide, (R)—N-(3-(2-(tert-butyl)-5-(2-((4-(1-((1-(4-(2,6-dioxopiperidin-3-yl)phenyl)piperidin-4-yl)methyl)piperidin-4-yl)phenyl)amino)pyrimidin-4-yl)thiazol-4-yl)-2-fluorophenyl)propane-1-sulfonamide, (S)—N-(3-(2-(tert-butyl)-5-(2-((4-(4-((1-(4-(2,6-dioxopiperidin-3-yl)phenyl)piperidin-4-yl)methyl)piperazin-1-yl)-3-fluorophenyl)amino)pyrimidin-4-yl)thiazol-4-yl)-2-fluorophenyl)propane-1-sulfonamide, (R)—N-(3-(2-(tert-butyl)-5-(2-((4-(4-((1-(4-(2,6-dioxopiperidin-3-yl)phenyl)piperidin-4-yl)methyl)piperazin-1-yl)-3-fluorophenyl)amino)pyrimidin-4-yl)thiazol-4-yl)-2-fluorophenyl)propane-1-sulfonamide, (2S,4R)-1-((S)-2-(5-(4-(4-((4-(2-(tert-butyl)-4-(2-fluoro-3-(propylsulfonamido)phenyl)thiazol-5-yl)pyrimidin-2-yl)amino)phenyl)piperidin-1-yl)-5-oxopentanamido)-3,3-dimethylbutanoyl)-4-hydroxy-N—((S)-1-(4-(4-methylthiazol-5-yl)phenyl)ethyl)pyrrolidine-2-carboxamide, (R)—N-(3-(2-(tert-butyl)-5-(2-((2-((1-(2-(2,6-dioxopiperidin-3-yl)-1-oxo-1,2-dihydroisoquinolin-6-yl)piperidin-4-yl)methyl)-1,2,3,4-tetrahydroisoquinolin-6-yl)amino)pyrimidin-4-yl)thiazol-4-yl)-2-fluorophenyl)propane-1-sulfonamide, N-(3-(2-(tert-butyl)-5-(2-((1-((1-(((1S,4r)-4-(4-((S)-2,6-dioxopiperidin-3-yl)phenoxy)cyclohexyl)methyl)piperidin-4-yl)methyl)-1H-pyrazol-4-yl)amino)pyrimidin-4-yl)thiazol-4-yl)-2-fluorophenyl)propane-1-sulfonamide, (R)—N-(3-(2-(tert-butyl)-5-(2-((1-((1-((1-(2-(2,6-dioxopiperidin-3-yl)-1-oxo-1,2-dihydroisoquinolin-6-yl)piperidin-4-yl)methyl)piperidin-4-yl)methyl)-1H-pyrazol-4-yl)amino)pyrimidin-4-yl)thiazol-4-yl)-2-fluorophenyl)propane-1-sulfonamide, (R)-5-(4-((4-((4-((4-(2-(tert-butyl)-4-(2-fluoro-3-(propylsulfonamido)phenyl)thiazol-5-yl)pyrimidin-2-yl)amino)-1H-pyrazol-1-yl)methyl)piperidin-1-yl)methyl)piperidin-1-yl)-N-(2,6-dioxopiperidin-3-yl)picolinamide, N-(3-(2-(tert-butyl)-5-(2-((1-((1-(2-((R)-1-(5-((S)-2,6-dioxopiperidin-3-yl)pyridin-2-yl)pyrrolidin-3-yl)ethyl)piperidin-4-yl)methyl)-1H-pyrazol-4-yl)amino)pyrimidin-4-yl)thiazol-4-yl)-2-fluorophenyl)propane-1-sulfonamide, (R)—N-(3-(2-(tert-butyl)-5-(2-((1-(1-((1-(2-(2,6-dioxopiperidin-3-yl)-1-oxo-1,2-dihydroisoquinolin-6-yl)piperidin-4-yl)methyl)piperidin-4-yl)-1H-pyrazol-4-yl)amino)pyrimidin-4-yl)thiazol-4-yl)-2-fluorophenyl)propane-1-sulfonamide, (R)-5-(4-((4-(4-((4-(2-(tert-butyl)-4-(2-fluoro-3-(propylsulfonamido)phenyl)thiazol-5-yl)pyrimidin-2-yl)amino)-1H-pyrazol-1-yl)piperidin-1-yl)methyl)piperidin-1-yl)-N-(2,6-dioxopiperidin-3-yl)picolinamide, N-(3-(2-(tert-butyl)-5-(2-((1-(1-(2-((R)-1-(5-((S)-2,6-dioxopiperidin-3-yl)pyridin-2-yl)pyrrolidin-3-yl)ethyl)piperidin-4-yl)-1H-pyrazol-4-yl)amino)pyrimidin-4-yl)thiazol-4-yl)-2-fluorophenyl)propane-1-sulfonamide, N-(3-(2-(tert-butyl)-5-(2-((1-(1-(2-((S)-1-(5-((S)-2,6-dioxopiperidin-3-yl)pyridin-2-yl)pyrrolidin-3-yl)ethyl)piperidin-4-yl)-1H-pyrazol-4-yl)amino)pyrimidin-4-yl)thiazol-4-yl)-2-fluorophenyl)propane-1-sulfonamide, (R)—N-(3-(2-(tert-butyl)-5-(2-((3-(1-((1-(2-(2,6-dioxopiperidin-3-yl)-1-oxo-1,2-dihydroisoquinolin-6-yl)piperidin-4-yl)methyl)piperidin-4-yl)phenyl)amino)pyrimidin-4-yl)thiazol-4-yl)-2-fluorophenyl)propane-1-sulfonamide, N-(3-(2-(tert-butyl)-5-(2-((3-(1-(2-((R)-1-(5-((S)-2,6-dioxopiperidin-3-yl)pyridin-2-yl)pyrrolidin-3-yl)ethyl)piperidin-4-yl)phenyl)amino)pyrimidin-4-yl)thiazol-4-yl)-2-fluorophenyl)propane-1-sulfonamide, N-(3-(2-(tert-butyl)-5-(2-((3-(1-(2-((S)-1-(5-((S)-2,6-dioxopiperidin-3-yl)pyridin-2-yl)pyrrolidin-3-yl)ethyl)piperidin-4-yl)phenyl)amino)pyrimidin-4-yl)thiazol-4-yl)-2-fluorophenyl)propane-1-sulfonamide, (S)—N-(3-(2-(tert-butyl)-5-(2-((3-(1-((1-(5-(2,6-dioxopiperidin-3-yl)pyridin-2-yl)-4-methylpiperidin-4-yl)methyl)piperidin-4-yl)phenyl)amino)pyrimidin-4-yl)thiazol-4-yl)-2-fluorophenyl)propane-1-sulfonamide, N-(3-(2-(tert-butyl)-5-(2-((3-((1-(((1S,4r)-4-(4-((S)-2,6-dioxopiperidin-3-yl)phenoxy)cyclohexyl)methyl)piperidin-4-yl)oxy)phenyl)amino)pyrimidin-4-yl)thiazol-4-yl)-2-fluorophenyl)propane-1-sulfonamide, (R)—N-(3-(2-(tert-butyl)-5-(2-((3-((1-((1-(2-(2,6-dioxopiperidin-3-yl)-1-oxo-1,2-dihydroisoquinolin-6-yl)piperidin-4-yl)methyl)piperidin-4-yl)oxy)phenyl)amino)pyrimidin-4-yl)thiazol-4-yl)-2-fluorophenyl)propane-1-sulfonamide, N-(3-(2-(tert-butyl)-5-(2-((3-((1-(2—((R)-1-(5-((S)-2,6-dioxopiperidin-3-yl)pyridin-2-yl)pyrrolidin-3-yl)ethyl)piperidin-4-yl)oxy)phenyl)amino)pyrimidin-4-yl)thiazol-4-yl)-2-fluorophenyl)propane-1-sulfonamide, N-(3-(2-(tert-butyl)-5-(2-((3-((1-(2-((S)-1-(5-((S)-2,6-dioxopiperidin-3-yl)pyridin-2-yl)pyrrolidin-3-yl)ethyl)piperidin-4-yl)oxy)phenyl)amino)pyrimidin-4-yl)thiazol-4-yl)-2-fluorophenyl)propane-1-sulfonamide, (S)—N-(3-(2-(tert-butyl)-5-(2-((3-((1-((1-(5-(2,6-dioxopiperidin-3-yl)pyridin-2-yl)-4-methylpiperidin-4-yl)methyl)piperidin-4-yl)oxy)phenyl)amino)pyrimidin-4-yl)thiazol-4-yl)-2-fluorophenyl)propane-1-sulfonamide, (R)-5-(4-((4-(4-((4-(2-(tert-butyl)-4-(2-fluoro-3-(propylsulfonamido)phenyl)thiazol-5-yl)pyrimidin-2-yl)amino)-1H-pyrazol-1-yl)piperidin-1-yl)methyl)piperidin-1-yl)-N-(2,6-dioxopiperidin-3-yl)picolinamide, (R)-5-(4-((4-((4-((4-(2-(tert-butyl)-4-(2-fluoro-3-(propylsulfonamido)phenyl)thiazol-5-yl)pyrimidin-2-yl)amino)-1H-pyrazol-1-yl)methyl)piperidin-1-yl)methyl)piperidin-1-yl)-N-(2,6-dioxopiperidin-3-yl)picolinamide, (S)—N-(3-(2-(tert-butyl)-5-(2-((4-(4-(1-(4-(2,6-dioxopiperidin-3-yl)phenyl)piperidine-4-carbonyl)piperazin-1-yl)-3-fluorophenyl)amino)pyrimidin-4-yl)thiazol-4-yl)-2-fluorophenyl)propane-1-sulfonamide, (S)—N-(3-(5-(2-((4-(1-((1-(4-(2,6-dioxopiperidin-3-yl)phenyl)piperidin-4-yl)methyl)piperidin-4-yl)phenyl)amino)pyrimidin-4-yl)-2-methylthiazol-4-yl)-2-fluorophenyl)propane-1-sulfonamide, (R)—N-(3-(5-(2-((4-(1-((1-(4-(2,6-dioxopiperidin-3-yl)phenyl)piperidin-4-yl)methyl)piperidin-4-yl)phenyl)amino)pyrimidin-4-yl)-2-methylthiazol-4-yl)-2-fluorophenyl)propane-1-sulfonamide, (S)—N-(3-(5-(2-((5-(4-((1-(5-(2,6-dioxopiperidin-3-yl)pyridin-2-yl)piperidin-4-yl)methyl)piperazin-1-yl)pyridin-2-yl)amino)pyrimidin-4-yl)-2-methylthiazol-4-yl)-2-fluorophenyl)propane-1-sulfonamide, (S)—N-(3-(5-(2-((5-(4-((1-(4-(2,6-dioxopiperidin-3-yl)phenyl)piperidin-4-yl)methyl)piperazin-1-yl)pyridin-2-yl)amino)pyrimidin-4-yl)-2-methylthiazol-4-yl)-2-fluorophenyl)propane-1-sulfonamide, (R)—N-(3-(5-(2-((5-(4-((1-(4-(2,6-dioxopiperidin-3-yl)phenyl)piperidin-4-yl)methyl)piperazin-1-yl)pyridin-2-yl)amino)pyrimidin-4-yl)-2-methylthiazol-4-yl)-2-fluorophenyl)propane-1-sulfonamide, (R)—N-(3-(2-(tert-butyl)-5-(2-((4-(4-((1-(4-(2,6-dioxopiperidin-3-yl)phenyl)piperidin-4-yl)methyl)piperazin-1-yl)phenyl)amino)pyrimidin-4-yl)thiazol-4-yl)-2-fluorophenyl)propane-1-sulfonamide, (S)—N-(3-(2-(tert-butyl)-5-(2-((4-(4-((1-(4-(2,6-dioxopiperidin-3-yl)phenyl)piperidin-4-yl)methyl)piperazin-1-yl)phenyl)amino)pyrimidin-4-yl)thiazol-4-yl)-2-fluorophenyl)propane-1-sulfonamide, (S)—N-(3-(2-(tert-butyl)-5-(2-((4-(4-(1-(5-(2,6-dioxopiperidin-3-yl)pyridin-2-yl)piperidine-4-carbonyl)piperazin-1-yl)-3-fluorophenyl)amino)pyrimidin-4-yl)thiazol-4-yl)-2-fluorophenyl)propane-1-sulfonamide, rac-(3R)-3-[6-(4-{4-[6-({4-[2-tert-butyl-4-(3-{[ethyl(methyl)sulfamoyl]amino}-2-fluorophenyl)-1,3-thiazol-5-yl]pyrimidin-2-yl}amino)pyridin-3-yl]piperazine-1-carbonyl}piperidin-1-yl)pyridin-3-yl]piperidine-2,6-dione, (S)—N-(3-(5-(2-((4-(1-(1-(5-(2,6-dioxopiperidin-3-yl)pyridin-2-yl)piperidine-4-carbonyl)piperidin-4-yl)phenyl)amino)pyrimidin-4-yl)-2-methylthiazol-4-yl)-2-fluorophenyl)propane-1-sulfonamide, N-(3-(2-(tert-butyl)-5-(2-((4-(4-((R)-1-(5-((R)-2,6-dioxopiperidin-3-yl)pyridin-2-yl)pyrrolidine-3-carbonyl)piperazin-1-yl)-3-fluorophenyl)amino)pyrimidin-4-yl)thiazol-4-yl)-2-fluorophenyl)propane-1-sulfonamide, (R)—N-(3-(5-(2-((4-(1-((1-(5-(2,6-dioxopiperidin-3-yl)pyridin-2-yl)piperidin-4-yl)methyl)piperidin-4-yl)phenyl)amino)pyrimidin-4-yl)-2-methylthiazol-4-yl)-2-fluorophenyl)propane-1-sulfonamide, N-(3-(2-(tert-butyl)-5-(2-((4-(4-((R)-1-(5-((R)-2,6-dioxopiperidin-3-yl)pyridin-2-yl)pyrrolidine-3-carbonyl)piperazin-1-yl)-3-fluorophenyl)amino)pyrimidin-4-yl)thiazol-4-yl)-2-fluorophenyl)propane-1-sulfonamide, rac-(3S)-3-[6-(3-{4-[6-({4-[2-tert-butyl-4-(3-{[ethyl(methyl)sulfamoyl]amino}-2-fluorophenyl)-1,3-thiazol-5-yl]pyrimidin-2-yl}amino)pyridin-3-yl]piperazine-1-carbonyl}azetidin-1-yl)pyridin-3-yl]piperidine-2,6-dione, (S)—N-(3-(2-(tert-butyl)-5-(2-((4-(4-(2-(1-(4-((2,6-dioxopiperidin-3-yl)amino)-2-fluorophenyl)-4-hydroxypiperidin-4-yl)acetyl)piperazin-1-yl)-3-fluorophenyl)amino)pyrimidin-4-yl)thiazol-4-yl)-2-fluorophenyl)propane-1-sulfonamide, (R)—N-(3-(2-(tert-butyl)-5-(2-((4-(4-(2-(6-(2,6-dioxopiperidin-3-yl)-3,4-dihydroisoquinolin-2(1H)-yl)acetyl)piperazin-1-yl)-3-fluorophenyl)amino)pyrimidin-4-yl)thiazol-4-yl)-2-fluorophenyl)propane-1-sulfonamide, N-(3-(2-(tert-butyl)-5-(2-((4-(4-((1S,4r)-4-((5-((S)-2,6-dioxopiperidin-3-yl)pyridin-2-yl)oxy)cyclohexane-1-carbonyl)piperazin-1-yl)-3-fluorophenyl)amino)pyrimidin-4-yl)thiazol-4-yl)-2-fluorophenyl)propane-1-sulfonamide, (R)—N-(3-(2-(tert-butyl)-5-(2-((4-(4-(2-(1-(5-(2,6-dioxopiperidin-3-yl)pyridin-2-yl)piperidin-4-yl)acetyl)piperazin-1-yl)-3-fluorophenyl)amino)pyrimidin-4-yl)thiazol-4-yl)-2-fluorophenyl)propane-1-sulfonamide, (S)—N-(3-(2-(tert-butyl)-5-(2-((4-(4-((1-(5-(2,6-dioxopiperidin-3-yl)pyridin-2-yl)piperidin-4-yl)methyl)piperazin-1-yl)phenyl)amino)pyrimidin-4-yl)thiazol-4-yl)-2-fluorophenyl)propane-1-sulfonamide, (S)—N-(3-(2-(tert-butyl)-5-(2-((4-(4-(2-(1-(4-((2,6-dioxopiperidin-3-yl)amino)phenyl)piperidin-4-yl)acetyl)piperazin-1-yl)-3-fluorophenyl)amino)pyrimidin-4-yl)thiazol-4-yl)-2-fluorophenyl)propane-1-sulfonamide, (S)—N-(3-(2-(tert-butyl)-5-(2-((4-(4-((1-(5-(2,6-dioxopiperidin-3-yl)pyridin-2-yl)piperidin-4-yl)methyl)piperazin-1-yl)-3-fluorophenyl)amino)pyrimidin-4-yl)thiazol-4-yl)-2-fluorophenyl)propane-1-sulfonamide, (S)—N-(3-(5-(2-((4-(1-(2-(1-(4-((2,6-dioxopiperidin-3-yl)amino)phenyl)piperidin-4-yl)acetyl)piperidin-4-yl)phenyl)amino)pyrimidin-4-yl)-2-methylthiazol-4-yl)-2-fluorophenyl)propane-1-sulfonamide, rac-(3S)-3-{6-[4-({4-[6-({4-[4-(3-{[ethyl(methyl)sulfamoyl]amino}-2-fluorophenyl)-2-methyl-1,3-thiazol-5-yl]pyrimidin-2-yl}amino)pyridin-3-yl]piperazin-1-yl}methyl)piperidin-1-yl]pyridin-3-yl}piperidine-2,6-dione, rac-(3S)-3-[6-(4-{4-[6-({4-[2-tert-butyl-4-(3-{[ethyl(methyl)sulfamoyl]amino}-2-fluorophenyl)-1,3-thiazol-5-yl]pyrimidin-2-yl}amino)pyridin-3-yl]piperazine-1-carbonyl}-4-methylpiperidin-1-yl)pyridin-3-yl]piperidine-2,6-dione, (3R)-3-{4-[4-({4-[6-({4-[4-(3-{[ethyl(methyl)sulfamoyl]amino}-2-fluorophenyl)-2-methyl-1,3-thiazol-5-yl]pyrimidin-2-yl}amino)pyridin-3-yl]piperazin-1-yl}methyl)piperidin-1-yl]phenyl}piperidine-2,6-dione, rac-(3S)-3-[6-(4-{4-[6-({4-[4-(3-{[ethyl(methyl)sulfamoyl]amino}-2-fluorophenyl)-2-methyl-1,3-thiazol-5-yl]pyrimidin-2-yl}amino)pyridin-3-yl]piperazine-1-carbonyl}piperidin-1-yl)pyridin-3-yl]piperidine-2,6-dione, rac-(3R)-3-{6-[4-(2-{4-[6-({4-[4-(3-{[ethyl(methyl)sulfamoyl]amino}-2-fluorophenyl)-2-methyl-1,3-thiazol-5-yl]pyrimidin-2-yl}amino)pyridin-3-yl]piperazin-1-yl}-2-oxoethyl)piperidin-1-yl]pyridin-3-yl}piperidine-2,6-dione, (S)—N-(3-(2-(tert-butyl)-5-(2-((4-(4-(1-(4-(2,6-dioxopiperidin-3-yl)phenyl)piperidine-4-carbonyl)piperazin-1-yl)phenyl)amino)pyrimidin-4-yl)thiazol-4-yl)-2-fluorophenyl)propane-1-sulfonamide, (S)—N-(3-(2-(tert-butyl)-5-(2-((4-(1-(1-(4-(2,6-dioxopiperidin-3-yl)phenyl)piperidine-4-carbonyl)piperidin-4-yl)phenyl)amino)pyrimidin-4-yl)thiazol-4-yl)-2-fluorophenyl)propane-1-sulfonamide, rac-(3S)-3-[4-(4-{4-[6-({4-[2-tert-butyl-4-(3-{[ethyl(methyl)sulfamoyl]amino}-2-fluorophenyl)-1,3-thiazol-5-yl]pyrimidin-2-yl}amino)pyridin-3-yl]piperazine-1-carbonyl}piperidin-1-yl)phenyl]piperidine-2,6-dione, (R)—N-(3-(2-(tert-butyl)-5-(2-((4-(1-((1-(5-(2,6-dioxopiperidin-3-yl)pyridin-2-yl)piperidin-4-yl)methyl)piperidin-4-yl)phenyl)amino)pyrimidin-4-yl)thiazol-4-yl)-2-fluorophenyl)propane-1-sulfonamide, rac-(3S)-3-{6-[4-({4-[6-({4-[4-(3-{[ethyl(methyl)sulfamoyl]amino}-2-fluorophenyl)-2-methyl-1,3-thiazol-5-yl]pyrimidin-2-yl}amino)pyridin-3-yl]piperazin-1-yl}methyl)-4-methylpiperidin-1-yl]pyridin-3-yl}piperidine-2,6-dione, rac-(3S)-3-{4-[4-({4-[6-({4-[4-(3-{[ethyl(methyl)sulfamoyl]amino}-2-fluorophenyl)-2-methyl-1,3-thiazol-5-yl]pyrimidin-2-yl}amino)pyridin-3-yl]piperazin-1-yl}methyl)-4-fluoropiperidin-1-yl]phenyl}piperidine-2,6-dione, (S)—N-(3-(5-(2-((4-(4-((1-(5-(2,6-dioxopiperidin-3-yl)pyridin-2-yl)-4-methylpiperidin-4-yl)methyl)piperazin-1-yl)phenyl)amino)pyrimidin-4-yl)-2-methylthiazol-4-yl)-2-fluorophenyl)propane-1-sulfonamide, (S)—N-(3-(5-(2-((4-(4-((1-(4-(2,6-dioxopiperidin-3-yl)phenyl)-4-fluoropiperidin-4-yl)methyl)piperazin-1-yl)phenyl)amino)pyrimidin-4-yl)-2-methylthiazol-4-yl)-2-fluorophenyl)propane-1-sulfonamide, rac-(3R)-3-{6-[6-({4-[6-({4-[2-tert-butyl-4-(3-{[ethyl(methyl)sulfamoyl]amino}-2-fluorophenyl)-1,3-thiazol-5-yl]pyrimidin-2-yl}amino)pyridin-3-yl]piperazin-1-yl}methyl)-2-azaspiro[3.3]heptan-2-yl]pyridin-3-yl}piperidine-2,6-dione, (3R)-3-{6-[4-({4-[6-({4-[2-tert-butyl-4-(3-{[ethyl(methyl)sulfamoyl]amino}-2-fluorophenyl)-1,3-thiazol-5-yl]pyrimidin-2-yl}amino)pyridin-3-yl]piperazin-1-yl}methyl)piperidin-1-yl]pyridin-3-yl}piperidine-2,6-dione, rac-(3R)-3-[2-(2-{4-[4-({4-[2-tert-butyl-4-(3-{[ethyl(methyl)sulfamoyl]amino}-2-fluorophenyl)-1,3-thiazol-5-yl]pyrimidin-2-yl}amino)-2-fluorophenyl]piperazin-1-yl}-2-oxoethyl)-1,2,3,4-tetrahydroisoquinolin-6-yl]piperidine-2,6-dione, and (3R)-3-{6-[4-({4-[6-({4-[4-(3-{[ethyl(methyl)sulfamoyl]amino}-2-fluorophenyl)-2-methyl-1,3-thiazol-5-yl]pyrimidin-2-yl}amino)pyridin-3-yl]piperazin-1-yl}methyl)piperidin-1-yl]pyridin-3-yl}piperidine-2,6-dione.

69. The compound of any one of claims 2, 10-17, 31-33, or 53-64 selected from the group consisting of (S)—N-(3-(1-(4-(4-((1-(4-(2,6-dioxopiperidin-3-yl)phenyl)piperidin-4-yl)methyl)piperazin-1-yl)phenyl)-3-(pyridin-4-yl)-1H-pyrazol-4-yl)-2-fluorophenyl)propane-1-sulfonamide, (R)—N-(3-(1-(4-(4-((1-(4-(2,6-dioxopiperidin-3-yl)phenyl)piperidin-4-yl)methyl)piperazin-1-yl)phenyl)-3-(pyridin-4-yl)-1H-pyrazol-4-yl)-2-fluorophenyl)propane-1-sulfonamide, and (S)—N-(3-(1-(4-(4-((1-(5-(2,6-dioxopiperidin-3-yl)pyridin-2-yl)piperidin-4-yl)methyl)piperazin-1-yl)phenyl)-3-(pyridin-4-yl)-1H-pyrazol-4-yl)-2-fluorophenyl)propane-1-sulfonamide.

70. The compound of any one of claims 2, 31-33, or 53-64 selected from the group consisting of (R)—N-(3-(1-(4-(4-(1-(4-(2,6-dioxopiperidin-3-yl)phenyl)piperidine-4-carbonyl)piperazine-1-carbonyl)phenyl)-3-(pyridin-4-yl)-1H-pyrazol-4-yl)-2-fluorophenyl)propane-1-sulfonamide, (S)—N-(3-(1-(4-(4-((1-(4-(2,6-dioxopiperidin-3-yl)phenyl)piperidin-4-yl)methyl)piperazine-1-carbonyl)phenyl)-3-(pyridin-4-yl)-1H-pyrazol-4-yl)-2-fluorophenyl)propane-1-sulfonamide, (S)—N-(3-(1-(4-(4-(1-(4-(2,6-dioxopiperidin-3-yl)phenyl)piperidine-4-carbonyl)piperazin-1-yl)phenyl)-3-(pyridin-4-yl)-1H-pyrazol-4-yl)-2-fluorophenyl)propane-1-sulfonamide, (R)—N-(3-(1-(4-((1-(1-(5-(2,6-dioxopiperidin-3-yl)pyridin-2-yl)piperidine-4-carbonyl)piperidin-4-yl)oxy)phenyl)-3-(pyridin-4-yl)-1H-pyrazol-4-yl)-2-fluorophenyl)propane-1-sulfonamide, (S)—N-(3-(1-(4-(4-((1-(5-(2,6-dioxopiperidin-3-yl)pyridin-2-yl)-4-methylpiperidin-4-yl)methyl)piperazin-1-yl)phenyl)-3-(pyridin-4-yl)-1H-pyrazol-4-yl)-2-fluorophenyl)propane-1-sulfonamide, (R)—N-(3-(1-(4-(4-((1-(4-(2,6-dioxopiperidin-3-yl)phenyl)piperidin-4-yl)methyl)piperazine-1-carbonyl)phenyl)-3-(pyridin-4-yl)-1H-pyrazol-4-yl)-2-fluorophenyl)propane-1-sulfonamide, (S)—N-(3-(1-(1-(1-(1-(4-(2,6-dioxopiperidin-3-yl)phenyl)piperidine-4-carbonyl)piperidine-4-carbonyl)piperidin-4-yl)-3-(pyridin-4-yl)-1H-pyrazol-4-yl)-2-fluorophenyl)propane-1-sulfonamide, (S)—N-(3-(1-(4-(4-((1-(4-(2,6-dioxopiperidin-3-yl)-3-fluorophenyl)piperidin-4-yl)methyl)piperazin-1-yl)phenyl)-3-(pyridin-4-yl)-1H-pyrazol-4-yl)-2-fluorophenyl)propane-1-sulfonamide, (S)—N-(3-(1-(4-(1-((1-(4-(2,6-dioxopiperidin-3-yl)-2-fluorophenyl)piperidin-4-yl)methyl)piperidin-4-yl)phenyl)-3-(pyridin-4-yl)-1H-pyrazol-4-yl)-2-fluorophenyl)propane-1-sulfonamide, (S)—N-(3-(1-(1-(1-(1-(5-(2,6-dioxopiperidin-3-yl)pyridin-2-yl)piperidine-4-carbonyl)piperidine-4-carbonyl)piperidin-4-yl)-3-(pyridin-4-yl)-1H-pyrazol-4-yl)-2-fluorophenyl)propane-1-sulfonamide, (S)—N-(3-(1-(4-(4-((1-(4-(2,6-dioxopiperidin-3-yl)-2-fluorophenyl)piperidin-4-yl)methyl)piperazin-1-yl)phenyl)-3-(pyridin-4-yl)-1H-pyrazol-4-yl)-2-fluorophenyl)propane-1-sulfonamide, (S)—N-(3-(1-(6-(4-((1-(4-(2,6-dioxopiperidin-3-yl)phenyl)piperidin-4-yl)methyl)piperazin-1-yl)pyridin-3-yl)-3-(pyridin-4-yl)-1H-pyrazol-4-yl)-2-fluorophenyl)propane-1-sulfonamide, (R)—N-(3-(1-(4-(4-(2-(1-(5-(2,6-dioxopiperidin-3-yl)pyridin-2-yl)piperidin-4-yl)acetyl)piperazine-1-carbonyl)phenyl)-3-(pyridin-4-yl)-1H-pyrazol-4-yl)-2-fluorophenyl)propane-1-sulfonamide, (S)—N-(3-(1-(1-(1-((1-(5-(2,6-dioxopiperidin-3-yl)pyridin-2-yl)piperidin-4-yl)methyl)piperidine-4-carbonyl)piperidin-4-yl)-3-(pyridin-4-yl)-1H-pyrazol-4-yl)-2-fluorophenyl)propane-1-sulfonamide, (S)—N-(3-(1-(4-(4-((1-(5-(2,6-dioxopiperidin-3-yl)pyridin-2-yl)piperidin-4-yl)methyl)piperazin-1-yl)phenyl)-3-(pyridin-4-yl)-1H-pyrazol-4-yl)-2-fluorophenyl)propane-1-sulfonamide, (S)—N-(3-(1-(6-(4-((1-(5-(2,6-dioxopiperidin-3-yl)pyridin-2-yl)piperidin-4-yl)methyl)piperazin-1-yl)pyridin-3-yl)-3-(pyridin-4-yl)-1H-pyrazol-4-yl)-2-fluorophenyl)propane-1-sulfonamide, (S)—N-(3-(1-(4-(1-((1-(4-(2,6-dioxopiperidin-3-yl)phenyl)piperidin-4-yl)methyl)piperidin-4-yl)phenyl)-3-(pyridin-4-yl)-1H-pyrazol-4-yl)-2-fluorophenyl)propane-1-sulfonamide, (S)—N-(3-(1-(4-(1-((1-(4-(2,6-dioxopiperidin-3-yl)phenyl)piperidin-4-yl)methyl)piperidin-4-yl)phenyl)-3-(pyridin-4-yl)-1H-pyrazol-4-yl)-2-fluorophenyl)propane-1-sulfonamide, (S)—N-(3-(1-(4-(1-((1-(5-(2,6-dioxopiperidin-3-yl)pyridin-2-yl)piperidin-4-yl)methyl)piperidin-4-yl)phenyl)-3-(pyridin-4-yl)-1H-pyrazol-4-yl)-2-fluorophenyl)propane-1-sulfonamide, (S)—N-(3-(1-(4-(4-((1-(4-(2,6-dioxopiperidin-3-yl)phenyl)piperidin-4-yl)methyl)piperazin-1-yl)phenyl)-3-(pyridin-4-yl)-1H-pyrazol-4-yl)-2-fluorophenyl)propane-1-sulfonamide, (S)—N-(3-(1-(1-(1-(1-(5-(2,6-dioxopiperidin-3-yl)pyridin-2-yl)piperidine-4-carbonyl)-4-methylpiperidine-4-carbonyl)piperidin-4-yl)-3-(pyridin-4-yl)-1H-pyrazol-4-yl)-2-fluorophenyl)propane-1-sulfonamide, (S)—N-(3-(1-(4-(1-((1-(4-(2,6-dioxopiperidin-3-yl)-3-fluorophenyl)piperidin-4-yl)methyl)piperidin-4-yl)phenyl)-3-(pyridin-4-yl)-1H-pyrazol-4-yl)-2-fluorophenyl)propane-1-sulfonamide, and (R)—N-(3-(1-(4-(4-((1-(5-(2,6-dioxopiperidin-3-yl)pyridin-2-yl)piperidin-4-yl)methyl)piperazin-1-yl)phenyl)-3-(pyridin-4-yl)-1H-pyrazol-4-yl)-2-fluorophenyl)propane-1-sulfonamide.

71. The compound of any one of claims 34, 37-45, or 50-54 selected from the group consisting of (S)—N-(3-(5-(2-aminopyrimidin-4-yl)-2-(1-(1-(1-(5-(2,6-dioxopiperidin-3-yl)pyridin-2-yl)piperidine-4-carbonyl)piperidine-4-carbonyl)piperidin-4-yl)thiazol-4-yl)-2-fluorophenyl)propane-1-sulfonamide, (S)—N-(3-(5-(2-aminopyrimidin-4-yl)-2-(1-(1-(1-(5-(2,6-dioxopiperidin-3-yl)pyridin-2-yl)-4-methylpiperidine-4-carbonyl)piperidine-4-carbonyl)piperidin-4-yl)thiazol-4-yl)-2-fluorophenyl)propane-1-sulfonamide, (S)—N-(3-(5-(2-aminopyrimidin-4-yl)-2-(1-(1-(1-(5-(2,6-dioxopiperidin-3-yl)pyridin-2-yl)piperidine-4-carbonyl)-4-methylpiperidine-4-carbonyl)piperidin-4-yl)thiazol-4-yl)-2-fluorophenyl)propane-1-sulfonamide, (R)—N-(3-(5-(2-aminopyrimidin-4-yl)-2-(1-(1-(1-(4-(2,6-dioxopiperidin-3-yl)phenyl)piperidine-4-carbonyl)-4-methylpiperidine-4-carbonyl)piperidin-4-yl)thiazol-4-yl)-2-fluorophenyl)propane-1-sulfonamide, (S)—N-(3-(2-(1-(1-(1-(4-(2,6-dioxopiperidin-3-yl)phenyl)piperidine-4-carbonyl)piperidine-4-carbonyl)piperidin-4-yl)-5-(pyrimidin-4-yl)thiazol-4-yl)-2-fluorophenyl)propane-1-sulfonamide, (S)—N-(3-(2-(1-(1-(1-(5-(2,6-dioxopiperidin-3-yl)pyridin-2-yl)piperidine-4-carbonyl)-4-methylpiperidine-4-carbonyl)piperidin-4-yl)-5-(pyrimidin-4-yl)thiazol-4-yl)-2-fluorophenyl)propane-1-sulfonamide, and (S)—N-(3-(2-(1-(1-(1-(5-(2,6-dioxopiperidin-3-yl)pyridin-2-yl)piperidine-4-carbonyl)-4-methylpiperidine-4-carbonyl)piperidin-4-yl)-5-(pyridin-4-yl)thiazol-4-yl)-2-fluorophenyl)propane-1-sulfonamide.

72. The compound of any one of claims 34, 37, 44, 45, or 50-64 selected from the group consisting of (R)—N-(3-(5-(2-aminopyrimidin-4-yl)-2-(3-(2-(4-(3-(2-(2,6-dioxopiperidin-3-yl)-1,3-dioxoisoindolin-4-yl)propyl)piperazin-1-yl)acetyl)-3-azaspiro[5.5]undecan-9-yl)thiazol-4-yl)-2-fluorophenyl)propane-1-sulfonamide, N-(3-(5-(2-aminopyrimidin-4-yl)-2-(1-((1S,4r)-4-((6-((S)-2,6-dioxopiperidin-3-yl)-3,4-dihydroisoquinolin-2(1H)-yl)methyl)cyclohexane-1-carbonyl)piperidin-4-yl)thiazol-4-yl)-2-fluorophenyl)propane-1-sulfonamide, (S)—N-(3-(2-(1-(1-((1-(5-(2,6-dioxopiperidin-3-yl)pyridin-2-yl)piperidin-4-yl)methyl)piperidine-4-carbonyl)piperidin-4-yl)-5-(pyrimidin-4-yl)thiazol-4-yl)-2-fluorophenyl)propane-1-sulfonamide, (R)—N-(3-(5-(2-aminopyrimidin-4-yl)-2-(1-(2-(4-(2-(2,6-dioxopiperidin-3-yl)-1,3-dioxoisoindolin-5-yl)piperazin-1-yl)acetyl)-4-methylpiperidin-4-yl)thiazol-4-yl)-2-fluorophenyl)propane-1-sulfonamide, (R)—N-(3-(5-(2-aminopyrimidin-4-yl)-2-(1-(2-(1′-(2-(2,6-dioxopiperidin-3-yl)-1,3-dioxoisoindolin-5-yl)-[1,4′-bipiperidin]-4-yl)acetyl)-4-methylpiperidin-4-yl)thiazol-4-yl)-2-fluorophenyl)propane-1-sulfonamide, (S)—N-(3-(5-(2-aminopyrimidin-4-yl)-2-(1-(1-((1-(5-(2,6-dioxopiperidin-3-yl)pyridin-2-yl)piperidin-4-yl)methyl)piperidine-4-carbonyl)-4-methylpiperidin-4-yl)thiazol-4-yl)-2-fluorophenyl)propane-1-sulfonamide, (S)—N-(3-(5-(2-aminopyrimidin-4-yl)-2-(1-((1-(2-(6-(2,6-dioxopiperidin-3-yl)-3,4-dihydroisoquinolin-2(1H)-yl)acetyl)piperidin-4-yl)methyl)piperidin-4-yl)thiazol-4-yl)-2-fluorophenyl)propane-1-sulfonamide, (S)—N-(3-(5-(2-aminopyrimidin-4-yl)-2-(3-((1-(4-(2,6-dioxopiperidin-3-yl)phenyl)piperidin-4-yl)methyl)-3-azaspiro[5.5]undecan-9-yl)thiazol-4-yl)-2-fluorophenyl)propane-1-sulfonamide, (S)—N-(3-(2-(1-(1-((1-(5-(2,6-dioxopiperidin-3-yl)pyridin-2-yl)piperidin-4-yl)methyl)piperidine-4-carbonyl)piperidin-4-yl)-5-(pyridin-4-yl)thiazol-4-yl)-2-fluorophenyl)propane-1-sulfonamide, (R)—N-(3-(5-(2-aminopyrimidin-4-yl)-2-(1-(2-(1′-(2-(2,6-dioxopiperidin-3-yl)-1,3-dioxoisoindolin-5-yl)-[1,4′-bipiperidin]-4-yl)acetyl)piperidin-4-yl)thiazol-4-yl)-2-fluorophenyl)propane-1-sulfonamide, (R)—N-(3-(5-(2-aminopyrimidin-4-yl)-2-(1-(2-(4-(2-(2,6-dioxopiperidin-3-yl)-1,3-dioxoisoindolin-5-yl)piperazin-1-yl)acetyl)piperidin-4-yl)thiazol-4-yl)-2-fluorophenyl)propane-1-sulfonamide, (R)—N-(3-(5-(2-aminopyrimidin-4-yl)-2-(2-(1-(1-(1-(2-(2,6-dioxopiperidin-3-yl)-1,3-dioxoisoindolin-5-yl)piperidin-4-yl)azetidine-3-carbonyl)piperidin-4-yl)propan-2-yl)thiazol-4-yl)-2-fluorophenyl)propane-1-sulfonamide, N-(3-(5-(2-aminopyrimidin-4-yl)-2-(1-((1-((1R,4r)-4-(3-((R)-2,6-dioxopiperidin-3-yl)phenoxy)cyclohexane-1-carbonyl)piperidin-4-yl)methyl)piperidin-4-yl)thiazol-4-yl)-2-fluorophenyl)propane-1-sulfonamide, (R)—N-(3-(5-(2-aminopyrimidin-4-yl)-2-(3-((1-(5-(2,6-dioxopiperidin-3-yl)pyridin-2-yl)piperidin-4-yl)methyl)-3-azaspiro[5.5]undecan-9-yl)thiazol-4-yl)-2-fluorophenyl)propane-1-sulfonamide, (S)—N-(3-(5-(2-aminopyrimidin-4-yl)-2-(1-(1-(2-(1-(4-((2,6-dioxopiperidin-3-yl)amino)-2-fluorophenyl)-4-hydroxypiperidin-4-yl)acetyl)azetidin-3-yl)piperidin-4-yl)thiazol-4-yl)-2-fluorophenyl)propane-1-sulfonamide, (S)—N-(3-(5-(2-aminopyrimidin-4-yl)-2-(4-(1-(2-(1-(5-(2,6-dioxopiperidin-3-yl)pyridin-2-yl)piperidin-4-yl)acetyl)piperidin-4-yl)phenyl)thiazol-4-yl)-2-fluorophenyl)propane-1-sulfonamide, (S)—N-(3-(5-(2-aminopyrimidin-4-yl)-2-(4-(4-((1-(4-(2,6-dioxopiperidin-3-yl)phenyl)piperidin-4-yl)methyl)piperazin-1-yl)phenyl)thiazol-4-yl)-2-fluorophenyl)propane-1-sulfonamide, (S)—N-(3-(5-(2-aminopyrimidin-4-yl)-2-(4-(1-(2-(1-(4-((2,6-dioxopiperidin-3-yl)amino)phenyl)piperidin-4-yl)acetyl)piperidin-4-yl)phenyl)thiazol-4-yl)-2-fluorophenyl)propane-1-sulfonamide, (S)—N-(3-(5-(2-aminopyrimidin-4-yl)-2-(4-(1-(1-(5-(2,6-dioxopiperidin-3-yl)pyridin-2-yl)piperidine-4-carbonyl)piperidin-4-yl)phenyl)thiazol-4-yl)-2-fluorophenyl)propane-1-sulfonamide, (S)—N-(3-(5-(2-aminopyrimidin-4-yl)-2-(4-(1-((1-(4-(2,6-dioxopiperidin-3-yl)phenyl)piperidin-4-yl)methyl)piperidin-4-yl)phenyl)thiazol-4-yl)-2-fluorophenyl)propane-1-sulfonamide, N-(3-(5-(2-aminopyrimidin-4-yl)-2-(3-(((1S,4r)-4-((5-((S)-2,6-dioxopiperidin-3-yl)pyridin-2-yl)oxy)cyclohexyl)methyl)-3-azaspiro[5.5]undecan-9-yl)thiazol-4-yl)-2-fluorophenyl)propane-1-sulfonamide, (R)—N-(3-(5-(2-aminopyrimidin-4-yl)-2-(4-(4-(1-(4-(2,6-dioxopiperidin-3-yl)phenyl)piperidine-4-carbonyl)piperazin-1-yl)phenyl)thiazol-4-yl)-2-fluorophenyl)propane-1-sulfonamide, (R)-5-(4-(9-(5-(2-aminopyrimidin-4-yl)-4-(2-fluoro-3-(propylsulfonamido)phenyl)thiazol-2-yl)-3-azaspiro[5.5]undecane-3-carbonyl)piperidin-1-yl)-N-(2,6-dioxopiperidin-3-yl)picolinamide, N-(3-(5-(2-aminopyrimidin-4-yl)-2-(3-(2-((S)-1-(5-((S)-2,6-dioxopiperidin-3-yl)pyridin-2-yl)pyrrolidin-3-yl)ethyl)-3-azaspiro[5.5]undecan-9-yl)thiazol-4-yl)-2-fluorophenyl)propane-1-sulfonamide, (S)—N-(3-(5-(2-aminopyrimidin-4-yl)-2-(1-(1-(2-(1-(4-(2,6-dioxopiperidin-3-yl)phenyl)piperidin-4-yl)acetyl)azetidin-3-yl)piperidin-4-yl)thiazol-4-yl)-2-fluorophenyl)propane-1-sulfonamide, (S)—N-(3-(5-(2-aminopyrimidin-4-yl)-2-(3-(1-(5-(2,6-dioxopiperidin-3-yl)pyridin-2-yl)piperidine-4-carbonyl)-3-azaspiro[5.5]undecan-9-yl)thiazol-4-yl)-2-fluorophenyl)propane-1-sulfonamide, (S)—N-(3-(5-(2-aminopyrimidin-4-yl)-2-(4-(1-((1-(5-(2,6-dioxopiperidin-3-yl)pyridin-2-yl)piperidin-4-yl)methyl)piperidin-4-yl)phenyl)thiazol-4-yl)-2-fluorophenyl)propane-1-sulfonamide, (S)—N-(3-(2-(1-(1-(1-(4-(2,6-dioxopiperidin-3-yl)phenyl)piperidine-4-carbonyl)piperidine-4-carbonyl)piperidin-4-yl)-5-(pyridin-4-yl)thiazol-4-yl)-2-fluorophenyl)propane-1-sulfonamide, (S)—N-(3-(2-(1-(1-((1-(5-(2,6-dioxopiperidin-3-yl)pyridin-2-yl)piperidin-4-yl)methyl)piperidine-4-carbonyl)piperidin-4-yl)-5-(2-(methylamino)pyrimidin-4-yl)thiazol-4-yl)-2-fluorophenyl)propane-1-sulfonamide, (R)—N-(3-(5-(2-aminopyrimidin-4-yl)-2-(2-(2-(1′-(2-(2,6-dioxopiperidin-3-yl)-1,3-dioxoisoindolin-5-yl)-[1,4′-bipiperidin]-4-yl)acetyl)-2-azaspiro[3.5]nonan-7-yl)thiazol-4-yl)-2-fluorophenyl)propane-1-sulfonamide, (S)—N-(3-(2-(1-(1-(1-(5-(2,6-dioxopiperidin-3-yl)pyridin-2-yl)piperidine-4-carbonyl)piperidine-4-carbonyl)piperidin-4-yl)-5-(pyridin-4-yl)thiazol-4-yl)-2-fluorophenyl)propane-1-sulfonamide, N-(3-(5-(2-aminopyrimidin-4-yl)-2-(1-((1-(((1S,4r)-4-(4-((S)-2,6-dioxopiperidin-3-yl)phenoxy)cyclohexyl)methyl)piperidin-4-yl)methyl)piperidin-4-yl)thiazol-4-yl)-2-fluorophenyl)propane-1-sulfonamide, (R)—N-(3-(5-(2-aminopyrimidin-4-yl)-2-(2-(1-(2-(2,6-dioxopiperidin-3-yl)-1,3-dioxoisoindolin-5-yl)piperidine-4-carbonyl)-2-azaspiro[3.5]nonan-7-yl)thiazol-4-yl)-2-fluorophenyl)propane-1-sulfonamide, (S)—N-(3-(2-(1-(1-(2-(1-(4-((2,6-dioxopiperidin-3-yl)amino)phenyl)piperidin-4-yl)acetyl)piperidine-4-carbonyl)piperidin-4-yl)-5-(pyrimidin-4-yl)thiazol-4-yl)-2-fluorophenyl)propane-1-sulfonamide, (S)—N-(3-(5-(2-aminopyrimidin-4-yl)-2-(2-(1-(1-(1-(5-(2,6-dioxopiperidin-3-yl)pyridin-2-yl)piperidine-4-carbonyl)piperidine-4-carbonyl)piperidin-4-yl)ethyl)thiazol-4-yl)-2-fluorophenyl)propane-1-sulfonamide, (R)—N-(3-(5-(2-aminopyrimidin-4-yl)-2-(2-(2-(4-(2-(2,6-dioxopiperidin-3-yl)-1,3-dioxoisoindolin-5-yl)piperazin-1-yl)acetyl)-2-azaspiro[3.5]nonan-7-yl)thiazol-4-yl)-2-fluorophenyl)propane-1-sulfonamide, (R)—N-(3-(5-(2-aminopyrimidin-4-yl)-2-(4-(4-(1-(5-(2,6-dioxopiperidin-3-yl)pyridin-2-yl)piperidine-4-carbonyl)piperazin-1-yl)phenyl)thiazol-4-yl)-2-fluorophenyl)propane-1-sulfonamide, (S)—N-(3-(2-(1-(1-(1-(5-(2,6-dioxopiperidin-3-yl)pyridin-2-yl)piperidine-4-carbonyl)piperidine-4-carbonyl)piperidin-4-yl)-5-(pyrimidin-4-yl)thiazol-4-yl)-2-fluorophenyl)propane-1-sulfonamide, (R)—N-(3-(5-(2-aminopyrimidin-4-yl)-2-(1-(1-(1-(4-(2,6-dioxopiperidin-3-yl)phenyl)piperidine-4-carbonyl)piperidine-4-carbonyl)piperidin-4-yl)thiazol-4-yl)-2-fluorophenyl)propane-1-sulfonamide, (R)—N-(3-(5-(2-aminopyrimidin-4-yl)-2-(4-(4-((1-(4-(2,6-dioxopiperidin-3-yl)phenyl)piperidin-4-yl)methyl)piperazin-1-yl)phenyl)thiazol-4-yl)-2-fluorophenyl)propane-1-sulfonamide, (S)—N-(3-(5-(2-aminopyrimidin-4-yl)-2-(1-((1-(1-(5-(2,6-dioxopiperidin-3-yl)pyridin-2-yl)piperidine-4-carbonyl)piperidin-4-yl)methyl)piperidin-4-yl)thiazol-4-yl)-2-fluorophenyl)propane-1-sulfonamide, (R)—N-(3-(5-(2-aminopyrimidin-4-yl)-2-(4-(4-(2-(1-(5-(2,6-dioxopiperidin-3-yl)pyridin-2-yl)piperidin-4-yl)acetyl)piperazin-1-yl)phenyl)thiazol-4-yl)-2-fluorophenyl)propane-1-sulfonamide, (R)—N-(3-(5-(2-aminopyrimidin-4-yl)-2-(3-(2-(1′-(2-(2,6-dioxopiperidin-3-yl)-1,3-dioxoisoindolin-5-yl)-[1,4′-bipiperidin]-4-yl)acetyl)-3-azaspiro[5.5]undecan-9-yl)thiazol-4-yl)-2-fluorophenyl)propane-1-sulfonamide, (S)—N-(3-(5-(2-aminopyrimidin-4-yl)-2-(1-(1-((1-(4-(2,6-dioxopiperidin-3-yl)phenyl)piperidin-4-yl)methyl)piperidine-4-carbonyl)-4-methylpiperidin-4-yl)thiazol-4-yl)-2-fluorophenyl)propane-1-sulfonamide, (S)—N-(3-(2-(1-(1-(1-(4-(2,6-dioxopiperidin-3-yl)phenyl)piperidine-4-carbonyl)-4-methylpiperidine-4-carbonyl)piperidin-4-yl)-5-(2-(isopropylamino)pyrimidin-4-yl)thiazol-4-yl)-2-fluorophenyl)propane-1-sulfonamide, (R)—N-(3-(5-(2-aminopyrimidin-4-yl)-2-(3-(2-(4-(2-(2,6-dioxopiperidin-3-yl)-1,3-dioxoisoindolin-5-yl)piperazin-1-yl)acetyl)-3-azaspiro[5.5]undecan-9-yl)thiazol-4-yl)-2-fluorophenyl)propane-1-sulfonamide, N-(3-(5-(2-aminopyrimidin-4-yl)-2-(1-((1-((1R,4r)-4-(4-((R)-2,6-dioxopiperidin-3-yl)phenoxy)cyclohexane-1-carbonyl)piperidin-4-yl)methyl)piperidin-4-yl)thiazol-4-yl)-2-fluorophenyl)propane-1-sulfonamide, (R)—N-(3-(5-(2-aminopyrimidin-4-yl)-2-(2-(6-(2-(2,6-dioxopiperidin-3-yl)-3-oxoisoindolin-5-yl)hexanoyl)-2-azaspiro[3.5]nonan-7-yl)thiazol-4-yl)-2-fluorophenyl)propane-1-sulfonamide, N-(3-(2-(1-(1-(1-(5-((S)-2,6-dioxopiperidin-3-yl)pyridin-2-yl)piperidine-4-carbonyl)piperidine-4-carbonyl)piperidin-4-yl)-5-(2-(((R)-1-hydroxypropan-2-yl)amino)pyrimidin-4-yl)thiazol-4-yl)-2-fluorophenyl)propane-1-sulfonamide, (S)—N-(3-(5-(2-aminopyrimidin-4-yl)-2-(4-(4-((1-(5-(2,6-dioxopiperidin-3-yl)pyridin-2-yl)piperidin-4-yl)methyl)piperazin-1-yl)phenyl)thiazol-4-yl)-2-fluorophenyl)propane-1-sulfonamide, rac-(3S)-3-{6-[4-(4-{4-[5-(2-aminopyrimidin-4-yl)-4-(3-{[ethyl(methyl)sulfamoyl]amino}-2-fluorophenyl)-1,3-thiazol-2-yl]piperidine-1-carbonyl}piperidine-1-carbonyl)piperidin-1-yl]pyridin-3-yl}piperidine-2,6-dione, rac-(3S)-3-{6-[4-(4-{4-[4-(3-{[ethyl(methyl)sulfamoyl]amino}-2-fluorophenyl)-5-{2-[(propan-2-yl)amino]pyrimidin-4-yl}-1,3-thiazol-2-yl]piperidine-1-carbonyl}piperidine-1-carbonyl)piperidin-1-yl]pyridin-3-yl}piperidine-2,6-dione, (S)—N-(3-(5-(2-aminopyrimidin-4-yl)-2-(1-(1-((1-(4-(2,6-dioxopiperidin-3-yl)phenyl)piperidin-4-yl)methyl)piperidine-4-carbonyl)piperidin-4-yl)thiazol-4-yl)-2-fluorophenyl)propane-1-sulfonamide, (S)—N-(3-(5-(2-aminopyrimidin-4-yl)-2-(1-(1-((1-(5-(2,6-dioxopiperidin-3-yl)pyridin-2-yl)piperidin-4-yl)methyl)piperidine-4-carbonyl)piperidin-4-yl)thiazol-4-yl)-2-fluorophenyl)propane-1-sulfonamide, (R)—N-(3-(5-(2-aminopyrimidin-4-yl)-2-(3-((1-(2-(2,6-dioxopiperidin-3-yl)-1-oxoisoindolin-5-yl)piperidin-4-yl)methyl)-3-azaspiro[5.5]undecan-9-yl)thiazol-4-yl)-2-fluorophenyl)propane-1-sulfonamide, (R)—N-(3-(5-(2-aminopyrimidin-4-yl)-2-(1-(2-(2-(2-(2,6-dioxopiperidin-3-yl)-1,3-dioxoisoindolin-5-yl)-2,7-diazaspiro[3.5]nonan-7-yl)acetyl)-4-methylpiperidin-4-yl)thiazol-4-yl)-2-fluorophenyl)propane-1-sulfonamide, (R)—N-(3-(5-(2-aminopyrimidin-4-yl)-2-(3-(1-(2-(2,6-dioxopiperidin-3-yl)-1,3-dioxoisoindolin-5-yl)piperidine-4-carbonyl)-3-azaspiro[5.5]undecan-9-yl)thiazol-4-yl)-2-fluorophenyl)propane-1-sulfonamide, (R)—N-(3-(2-(1-(2-(2-(2-(2,6-dioxopiperidin-3-yl)-1,3-dioxoisoindolin-5-yl)-2,7-diazaspiro[3.5]nonan-7-yl)acetyl)piperidin-4-yl)-5-(2-(methylamino)pyrimidin-4-yl)thiazol-4-yl)-2-fluorophenyl)propane-1-sulfonamide, (R)—N-(3-(5-(2-aminopyrimidin-4-yl)-2-(3-(6-(2-(2,6-dioxopiperidin-3-yl)-3-oxoisoindolin-5-yl)hexanoyl)-3-azaspiro[5.5]undecan-9-yl)thiazol-4-yl)-2-fluorophenyl)propane-1-sulfonamide, (R)—N-(3-(5-(2-aminopyrimidin-4-yl)-2-(1-(1-(2-(1-(5-(2,6-dioxopiperidin-3-yl)pyridin-2-yl)piperidin-4-yl)acetyl)-4-methylpiperidine-4-carbonyl)piperidin-4-yl)thiazol-4-yl)-2-fluorophenyl)propane-1-sulfonamide, (S)—N-(3-(2-(1-(1-(1-(5-(2,6-dioxopiperidin-3-yl)pyridin-2-yl)piperidine-4-carbonyl)piperidine-4-carbonyl)piperidin-4-yl)-5-(2-(methylamino)pyrimidin-4-yl)thiazol-4-yl)-2-fluorophenyl)propane-1-sulfonamide, (R)—N-(3-(5-(2-aminopyrimidin-4-yl)-2-(1-(1-(2-(1-(5-(2,6-dioxopiperidin-3-yl)pyridin-2-yl)piperidin-4-yl)acetyl)piperidine-4-carbonyl)piperidin-4-yl)thiazol-4-yl)-2-fluorophenyl)propane-1-sulfonamide, (S)—N-(3-(2-(1-(1-(1-(5-(2,6-dioxopiperidin-3-yl)pyridin-2-yl)piperidine-4-carbonyl)piperidine-4-carbonyl)piperidin-4-yl)-5-(2-(isopropylamino)pyrimidin-4-yl)thiazol-4-yl)-2-fluorophenyl)propane-1-sulfonamide, (S)—N-(3-(5-(2-(cyclopropylamino)pyrimidin-4-yl)-2-(1-(1-(1-(5-(2,6-dioxopiperidin-3-yl)pyridin-2-yl)piperidine-4-carbonyl)piperidine-4-carbonyl)piperidin-4-yl)thiazol-4-yl)-2-fluorophenyl)propane-1-sulfonamide, (S)—N-(3-(2-(1-(1-(1-(4-(2,6-dioxopiperidin-3-yl)phenyl)piperidine-4-carbonyl)-4-methylpiperidine-4-carbonyl)piperidin-4-yl)-5-(pyrimidin-4-yl)thiazol-4-yl)-2-fluorophenyl)propane-1-sulfonamide, N-(3-(5-(2-aminopyrimidin-4-yl)-2-(3-(2-((R)-1-(4-((S)-2,6-dioxopiperidin-3-yl)phenyl)pyrrolidin-3-yl)acetyl)-3-azaspiro[5.5]undecan-9-yl)thiazol-4-yl)-2-fluorophenyl)propane-1-sulfonamide, (R)—N-(3-(5-(2-aminopyrimidin-4-yl)-2-(2-(2-(2-(2-(2,6-dioxopiperidin-3-yl)-1,3-dioxoisoindolin-5-yl)-2,7-diazaspiro[3.5]nonan-7-yl)acetyl)-2-azaspiro[3.5]nonan-7-yl)thiazol-4-yl)-2-fluorophenyl)propane-1-sulfonamide, (R)—N-(3-(5-(2-aminopyrimidin-4-yl)-2-(1-((1-(2-(1-(5-(2,6-dioxopiperidin-3-yl)pyridin-2-yl)piperidin-4-yl)acetyl)piperidin-4-yl)methyl)piperidin-4-yl)thiazol-4-yl)-2-fluorophenyl)propane-1-sulfonamide, (R)—N-(3-(5-(2-aminopyrimidin-4-yl)-2-(1-(2-(2-(2-(2,6-dioxopiperidin-3-yl)-1,3-dioxoisoindolin-5-yl)-2,7-diazaspiro[3.5]nonan-7-yl)acetyl)piperidin-4-yl)thiazol-4-yl)-2-fluorophenyl)propane-1-sulfonamide, (R)—N-(3-(5-(2-aminopyrimidin-4-yl)-2-(3-(1-(1-(2,6-dioxopiperidin-3-yl)-3-methyl-2-oxo-2,3-dihydro-1H-benzo[d]imidazol-4-yl)piperidine-4-carbonyl)-3-azaspiro[5.5]undecan-9-yl)thiazol-4-yl)-2-fluorophenyl)propane-1-sulfonamide, (R)—N-(3-(2-(1-(1-((1-(4-(2,6-dioxopiperidin-3-yl)phenyl)piperidin-4-yl)methyl)piperidine-4-carbonyl)piperidin-4-yl)-5-(2-(methylamino)pyrimidin-4-yl)thiazol-4-yl)-2-fluorophenyl)propane-1-sulfonamide, (R)—N-(3-(5-(2-aminopyrimidin-4-yl)-2-(1-(1-(1-(2-(2,6-dioxopiperidin-3-yl)-1,3-dioxoisoindolin-5-yl)piperidin-4-yl)azetidine-3-carbonyl)piperidin-4-yl)thiazol-4-yl)-2-fluorophenyl)propane-1-sulfonamide, (R)—N-(3-(5-(2-aminopyrimidin-4-yl)-2-(2-(1-(1-(2-(2,6-dioxopiperidin-3-yl)-1,3-dioxoisoindolin-5-yl)piperidin-4-yl)azetidine-3-carbonyl)-2-azaspiro[3.5]nonan-7-yl)thiazol-4-yl)-2-fluorophenyl)propane-1-sulfonamide, (S)—N-(3-(5-(2-aminopyrimidin-4-yl)-2-(1-(1-((1-(1-(2,6-dioxopiperidin-3-yl)-3-methyl-2-oxo-2,3-dihydro-1H-benzo[d]imidazol-4-yl)piperidin-4-yl)methyl)piperidine-4-carbonyl)piperidin-4-yl)thiazol-4-yl)-2-fluorophenyl)propane-1-sulfonamide, (R)—N-(3-(5-(2-aminopyrimidin-4-yl)-2-(3-(2-(2-(2-(2,6-dioxopiperidin-3-yl)-1,3-dioxoisoindolin-5-yl)-2,7-diazaspiro[3.5]nonan-7-yl)acetyl)-3-azaspiro[5.5]undecan-9-yl)thiazol-4-yl)-2-fluorophenyl)propane-1-sulfonamide, (R)—N-(3-(5-(2-aminopyrimidin-4-yl)-2-(2-(1-(2-(2-(2-(2,6-dioxopiperidin-3-yl)-1,3-dioxoisoindolin-5-yl)-2,7-diazaspiro[3.5]nonan-7-yl)acetyl)piperidin-4-yl)propan-2-yl)thiazol-4-yl)-2-fluorophenyl)propane-1-sulfonamide, and (R)-4-((4-(5-(2-aminopyrimidin-4-yl)-4-(2-fluoro-3-(propylsulfonamido)phenyl)thiazol-2-yl)piperidin-1-yl)methyl)-N-(2,6-dioxopiperidin-3-yl)benzamide.

73. The compound of claim 1, wherein X¹ is carbon; X² is nitrogen, X³ is nitrogen; X⁴ is carbon; and X⁵ is hydrogen.

74. The compound of claim 73, wherein
R¹ is alkyl, dialkylamino, cycloalkyl, or heterocycloalkyl each unsubstituted or substituted with one or more halogen;
R^{1a} is halogen;
R^{1b} is hydrogen, haloalkyl or halogen;
R² is —[Ar¹—Z¹]ₙ—Z²-Cy¹—Z³-L-UBM;
Ar¹ is arylene or heteroarylene, each unsubstituted or substituted with one or more halogen;
Z¹ is a bond;
n is one;
Z² is absent;
Cy¹ is absent or heterocycloalkylene, unsubstituted or substituted with one or more alkyl or one or more halogen;
Z³ is absent or C₁₋₁₀ alkylene;
L is a linker according to -L¹-L²-L³-L⁴- or -L⁴-L³-L²-L¹-, wherein
-L¹- is -Q¹- or -Q²-;
each -L²-, -L³-, and -L⁴- is independently, absent, —C₁₋₈ alkylene-, or -Q¹-;
each -Q¹- is a three- to seven-membered heterocycloalkylene comprising at least one nitrogen and is unsubstituted or substituted with one or more methyl or halogen;
each -Q²- is a five- to thirteen-membered bicyclic heterocycloalkylene comprising at least one nitrogen, wherein the five- to thirteen-membered bicyclic heterocycloalkylene is optionally a spiro bicyclic heterocycloalkylene ring; and
UBM is a ubiquitin ligase binding moiety; or
a stereoisomer and/or pharmaceutical salt thereof.

75. The compound of claim 73 or 74, wherein R¹ is selected from the group consisting of propyl, ethylmethylamino, sec-butyl,

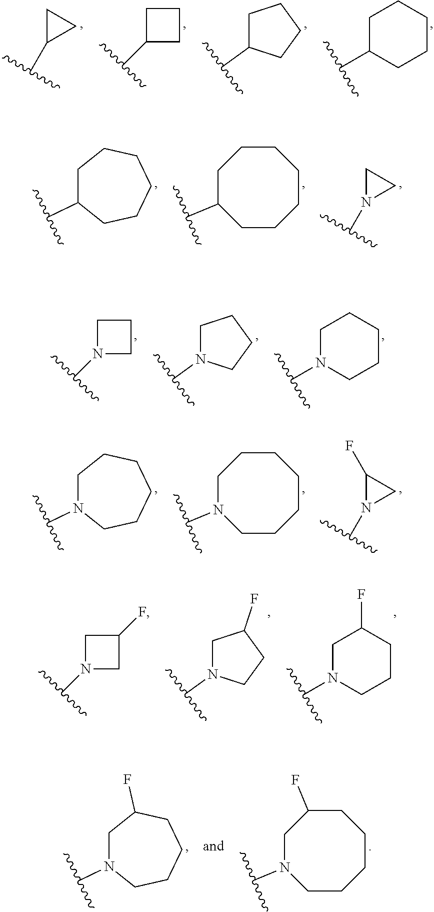

76. The compound of any one of claims 73-75, wherein R^{1a} is chloro or fluoro; and R^{1b} is hydrogen, chloro, fluoro, or haloalkyl.

77. The compound of any one of claims 73-76, wherein Ar¹ is arylene or heteroarylene unsubstituted or substituted with one or two halogen.

78. The compound of any one of claims 73-77, wherein UBM binds an E3 ubiquitin ligase.

79. The compound of claim 78, wherein UBM binds SCFP-TRCP, VHL, MDM2, IAP, or CRBN.

80. The compound of claim 79, wherein UBM binds CRBN.

81. The compound of claim 80, wherein UBM binds CRBN and has the following chemical formula 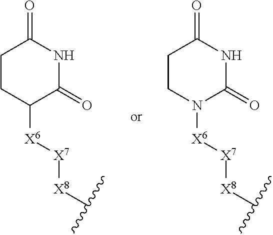
wherein
X⁶ absent or NR³;
X⁷ absent or —C(O)—; and
X⁸ is arylene or heteroarylene.

82. The compound of claim 81, wherein UBM binds CRBN and is selected from the group consisting of 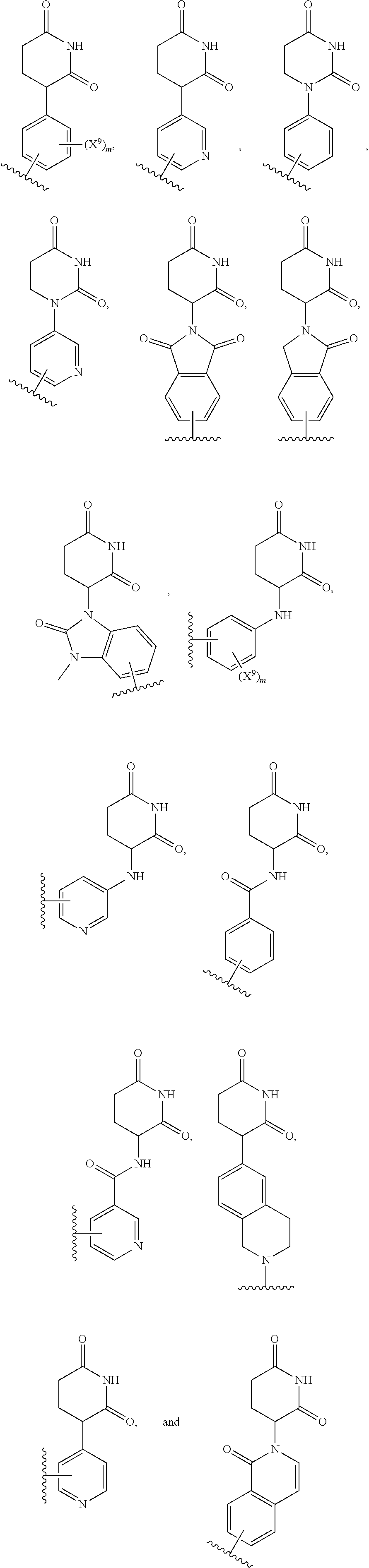
wherein
X⁹ is absent or halogen; and
m is one, two, three, or four.

83. The compound of any one of claims 1 or 73-82, wherein the compound is selected from the group consisting of (R)—N-(5-chloro-3-(1-(4-(4-((1-(5-(2,4-dioxotetrahydropyrimidin-1(2H)-yl)pyridin-2-yl)piperidin-4-yl)methyl)piperazin-1-yl)phenyl)-3-(pyridin-4-yl)-1H-pyrazol-4-yl)-2-fluorophenyl)-3-fluoropyrrolidine-1-sulfonamide; (R)—N-(5-chloro-3-(1-(4-(4-((1-(5-((R)-2,6-dioxopiperidin-3-yl)pyridin-2-yl)piperidin-4-yl)methyl)piperazin-1-yl)phenyl)-3-(pyridin-4-yl)-1H-pyrazol-4-yl)-2-fluorophenyl)-3-fluoropyrrolidine-1-sulfonamide; rac-(R)—N-(5-chloro-3-(1-(4-(4-((1-(5-(2,6-dioxopiperidin-3-yl)pyridin-2-yl)piperidin-4-yl)methyl)piperazin-1-yl)phenyl)-3-(pyridin-4-yl)-1H-pyrazol-4-yl)-2-fluorophenyl)pyrrolidine-1-sulfonamide; rac-(R)—N-(5-chloro-3-(1-(4-(4-((1-(5-(2,6-dioxopiperidin-3-yl)pyridin-2-yl)piperidin-4-yl)methyl)piperazin-1-yl)-2-fluorophenyl)-3-(pyridin-4-yl)-1H-pyrazol-4-yl)-2-fluorophenyl)pyrrolidine-1-sulfonamide; rac-(R)—N-(3-(1-(4-(4-((1-(5-(2,6-dioxopiperidin-3-yl)pyridin-2-yl)piperidin-4-yl)methyl)piperazin-1-yl)phenyl)-3-(pyridin-4-yl)-1H-pyrazol-4-yl)-2,5-difluorophenyl)pyrrolidine-1-sulfonamide;
N-(5-chloro-3-(1-(4-((R)-4-((1-(5-((RS)-2,6-dioxopiperidin-3-yl)pyridin-2-yl)piperidin-4-yl)methyl)-3-methylpiperazin-1-yl)phenyl)-3-(pyridin-4-yl)-1H-pyrazol-4-yl)-2-fluorophenyl)pyrrolidine-1-sulfonamide; (R)—N-(3-(1-(4-(4-((1-(4-(2,6-dioxopiperidin-3-yl)-2-fluorophenyl)piperidin-4-yl)methyl)piperazin-1-yl)-2-fluorophenyl)-3-(pyridin-4-yl)-1H-pyrazol-4-yl)-2,5-difluorophenyl)cyclopentanesulfonamide;
rac-(R)—N-(3-(1-(4-(4-((1-(5-(2,6-dioxopiperidin-3-yl)pyridin-2-yl)piperidin-4-yl)methyl)piperazin-1-yl)-3-fluorophenyl)-3-(pyridin-4-yl)-1H-pyrazol-4-yl)-2,5-difluorophenyl)cyclopentanesulfonamide;
rac-(R)—N-(5-chloro-3-(1-(4-(4-((1-(5-(2,6-dioxopiperidin-3-yl)pyridin-2-yl)piperidin-4-yl)methyl)piperazin-1-yl)-3-fluorophenyl)-3-(pyridin-4-yl)-1H-pyrazol-4-yl)-2-fluorophenyl)cyclopentanesulfonamide;
(R)—N-(5-chloro-3-(1-(4-(4-((1-(4-(2,6-dioxopiperidin-3-yl)-2-fluorophenyl)piperidin-4-yl)methyl)piperazin-1-yl)-3-fluorophenyl)-3-(pyridin-4-yl)-1H-pyrazol-4-yl)-2-fluorophenyl)cyclopentanesulfonamide;
(R)—N-(5-chloro-3-(1-(4-(4-((1-(4-(2,6-dioxopiperidin-3-yl)-2-fluorophenyl)piperidin-4-yl)methyl)-1,4-diazepan-1-yl)-2-fluorophenyl)-3-(pyridin-4-yl)-1H-pyrazol-4-yl)-2-fluorophenyl)pyrrolidine-1-sulfonamide;
(R)—N-(3-(1-(4-(4-((1-(4-(2,6-dioxopiperidin-3-yl)-2-fluorophenyl)piperidin-4-yl)methyl)-1,4-diazepan-1-yl)-2-fluorophenyl)-3-(pyridin-4-yl)-1H-pyrazol-4-yl)-2,5-difluorophenyl)pyrrolidine-1-sulfonamide; N-[5-chloro-3-(1-{4-[(3R)-4-[(1-{4-[(3R)-2,6-dioxopiperidin-3-yl]phenyl}piperidin-4-yl)methyl]-3-methylpiperazin-1-yl]phenyl}-3-(pyridin-4-yl)-1H-pyrazol-4-yl)-2-fluorophenyl]propane-1-sulfonamide; N-[5-chloro-3-(1-{4-[(3R)-4-[(1-{5-[(3RS)-2,6-dioxopiperidin-3-yl]pyridin-2-yl}piperidin-4-yl)methyl]-3-methylpiperazin-1-yl]phenyl}-3-(pyridin-4-yl)-1H-pyrazol-4-yl)-2-fluorophenyl]propane-1-sulfonamide; N-[5-chloro-3-(1-{4-[(3R)-4-({1-[4-(2,4-dioxo-1,3-diazinan-1-yl)phenyl]piperidin-4-yl}methyl)-3-methylpiperazin-1-yl]phenyl}-3-(pyridin-4-yl)-1H-pyrazol-4-yl)-2-fluorophenyl]propane-1-sulfonamide; N-[5-chloro-3-(1-{4-[(3S)-4-[(1-{4-[(3R)-2,6-dioxopiperidin-3-yl]phenyl}piperidin-4-yl)methyl]-3-methylpiperazin-1-yl]phenyl}-3-(pyridin-4-yl)-1H-pyrazol-4-yl)-2-fluorophenyl]propane-1-sulfonamide; N-[5-chloro-3-(1-{4-[(3S)-4-[(1-{5-[(3RS)-2,6-dioxopiperidin-3-yl]pyridin-2-yl}piperidin-4-yl)methyl]-3-methylpiperazin-1-yl]phenyl}-3-(pyridin-4-yl)-1H-pyrazol-4-yl)-2-fluorophenyl]propane-1-sulfonamide; N-[5-chloro-3-(1-{4-[(3S)-4-({1-[4-(2,4-dioxo-1,3-diazinan-1-yl)phenyl]piperidin-4-yl}methyl)-3-methylpiperazin-1-yl]phenyl}-3-(pyridin-4-yl)-1H-pyrazol-4-yl)-2-fluorophenyl]propane-1-sulfonamide; N-{5-chloro-3-[1-(4-{4-[(1-{4-[(3R)-2,6-dioxopiperidin-3-yl]phenyl}piperidin-4-yl)methyl]piperazin-1-yl}-2-fluorophenyl)-3-(pyridin-4-yl)-1H-pyrazol-4-yl]-2-fluorophenyl}pyrrolidine-1-sulfonamide; N-{5-chloro-3-[1-(4-{4-[(1-{4-[(3R)-2,6-dioxopiperidin-3-yl]phenyl}piperidin-4-yl)methyl]piperazin-1-yl}phenyl)-3-(pyridin-4-yl)-1H-pyrazol-4-yl]-2-fluorophenyl}pyrrolidine-1-sulfonamide; N-[5-chloro-3-(1-{4-[4-({1-[4-(2,4-dioxo-1,3-diazinan-1-yl)phenyl]piperidin-4-yl}methyl)piperazin-1-yl]phenyl}-3-(pyridin-4-yl)-1H-pyrazol-4-yl)-2-fluorophenyl]pyrrolidine-1-sulfonamide; rac-N-{5-chloro-3-[1-(4-{4-[(1-{4-[(3R)-2,6-dioxopiperidin-3-yl]phenyl}-4-fluoropiperidin-4-yl)methyl]piperazin-1-yl}phenyl)-3-(pyridin-4-yl)-1H-pyrazol-4-yl]-2-fluorophenyl}pyrrolidine-1-sulfonamide; N-{5-chloro-3-[1-(4-{4-[(1-{5-[(3R)-2,6-dioxopiperidin-3-yl]pyridin-2-yl}piperidin-4-yl)methyl]piperazin-1-yl}phenyl)-3-(pyridin-4-yl)-1H-pyrazol-4-yl]-2-fluorophenyl}pyrrolidine-1-sulfonamide;
N-{5-chloro-3-[1-(5-{4-[(1-{4-[(3R)-2,6-dioxopiperidin-3-yl]phenyl}piperidin-4-yl)methyl]piperazin-1-yl}pyridin-2-yl)-3-(pyridin-4-yl)-1H-pyrazol-4-yl]-2-fluorophenyl}pyrrolidine-1-sulfonamide; N-[5-chloro-3-(1-{5-[4-({1-[4-(2,4-dioxo-1,3-diazinan-1-yl)phenyl]piperidin-4-yl}methyl)piperazin-1-yl]pyridin-2-yl}-3-(pyridin-4-yl)-1H-pyrazol-4-yl)-2-fluorophenyl]pyrrolidine-1-sulfonamide; rac-N-{5-chloro-3-[1-(5-{4-[(1-{5-[(3R)-2,6-dioxopiperidin-3-yl]pyridin-2-yl}piperidin-4-yl)methyl]piperazin-1-yl}pyridin-2-yl)-3-(pyridin-4-yl)-1H-pyrazol-4-yl]-2-fluorophenyl}pyrrolidine-1-sulfonamide; N-(3-(1-(4-((R)-4-((1-(5-((RS)-2,6-dioxopiperidin-3-yl)pyridin-2-yl)piperidin-4-yl)methyl)-3-methylpiperazin-1-yl)-2-fluorophenyl)-3-(pyridin-4-yl)-1H-pyrazol-4-yl)-2,5-difluorophenyl)pyrrolidine-1-sulfonamide; N-{5-chloro-3-[1-(5-{4-[(1-{5-[(3R)-2,6-dioxopiperidin-3-yl]pyridin-2-yl}piperidin-4-yl)methyl]piperazin-1-yl}pyridin-2-yl)-3-(pyridin-4-yl)-1H-pyrazol-4-yl]-2-fluorophenyl}pyrrolidine-1-sulfonamide; (3R)—N-{5-chloro-3-[1-(4-{4-[(1-{4-[(3R)-2,6-dioxopiperidin-3-yl]phenyl}piperidin-4-yl)methyl]piperazin-1-yl}phenyl)-3-(pyridin-4-yl)-1H-pyrazol-4-yl]-2-fluorophenyl}-3-fluoropyrrolidine-1-sulfonamide; (3R)—N-[5-chloro-3-(1-{4-[4-({1-[4-(2,4-dioxo-1,3-diazinan-1-yl)phenyl]piperidin-4-yl}methyl)piperazin-1-yl]phenyl}-3-(pyridin-4-yl)-1H-pyrazol-4-yl)-2-fluorophenyl]-3-fluoropyrrolidine-1-sulfonamide; (3R)—N-{5-chloro-3-[1-(4-{4-[(1-{5-[(3R)-2,6-dioxopiperidin-3-yl]pyridin-2-yl}piperidin-4-yl)methyl]piperazin-1-yl}phenyl)-3-(pyridin-4-yl)-1H-pyrazol-4-yl]-2-fluorophenyl}-3-fluoropyrrolidine-1-sulfonamide; (3R)—N-{5-chloro-3-[1-(5-{4-[(1-{4-[(3R)-2,6-dioxopiperidin-3-yl]phenyl}piperidin-4-yl)methyl]piperazin-1-yl}pyridin-2-yl)-3-(pyridin-4-yl)-1H-pyrazol-4-yl]-2-fluorophenyl}-3-fluoropyrrolidine-1-sulfonamide; (3R)—N-[5-chloro-3-(1-{5-[4-({1-[4-(2,4-dioxo-1,3-diazinan-1-yl)phenyl]piperidin-4-yl}methyl)piperazin-1-yl]pyridin-2-yl}-3-(pyridin-4-yl)-1H-pyrazol-4-yl)-2-fluorophenyl]-3-fluoropyrrolidine-1-sulfonamide; (3R)—N-{5-chloro-3-[1-(5-{4-[(1-{5-[(3RS)-2,6-dioxopiperidin-3-yl]pyridin-2-yl}piperidin-4-yl)methyl]piperazin-1-yl}pyridin-2-yl)-3-(pyridin-4-yl)-1H-pyrazol-4-yl]-2-fluorophenyl}-3-fluoropyrrolidine-1-sulfonamide; (3R)—N-{5-chloro-3-[1-(5-{4-[(1-{5-[(3R)-2,6-dioxopiperidin-3-yl]pyridin-2-yl}piperidin-4-yl)methyl]piperazin-1-yl}pyridin-2-yl)-3-(pyridin-4-yl)-1H-pyrazol-4-yl]-2-fluorophenyl}-3-fluoropyrrolidine-1-sulfonamide; (3R)—N-{5-chloro-3-[1-(5-{4-[(7-{4-[(3RS)-2,6-dioxopiperidin-3-yl]phenyl}-7-azaspiro[3.5]nonan-2-yl)methyl]piperazin-1-yl}pyridin-2-yl)-3-(pyridin-4-yl)-1H-pyrazol-4-yl]-2-fluorophenyl}-3-fluoropyrrolidine-1-sulfonamide; N-{3-[1-(4-{4-[(1-{4-[(3R)-2,6-dioxopiperidin-3-yl]phenyl}piperidin-4-yl)methyl]piperazin-1-yl}phenyl)-3-(pyridin-4-yl)-1H-pyrazol-4-yl]-2,5-difluorophenyl}pyrrolidine-1-sulfonamide; N-[3-(1-{4-[4-({1-[4-(2,4-dioxo-1,3-diazinan-1-yl)phenyl]piperidin-4-yl}methyl)piperazin-1-yl]phenyl}-3-(pyridin-4-yl)-1H-pyrazol-4-yl)-2,5-difluorophenyl]pyrrolidine-1-sulfonamide; N-{3-[1-(4-{4-[(1-{5-[(3R)-2,6-dioxopiperidin-3-yl]pyridin-2-yl}piperidin-4-yl)methyl]piperazin-1-yl}phenyl)-3-(pyridin-4-yl)-1H-pyrazol-4-yl]-2,5-difluorophenyl}pyrrolidine-1-sulfonamide; (3R)—N-{3-[1-(5-{4-[(1-{4-[(3R)-2,6-dioxopiperidin-3-yl]phenyl}piperidin-4-yl)methyl]piperazin-1-yl}pyridin-2-yl)-3-(pyridin-4-yl)-1H-pyrazol-4-yl]-2,5-difluorophenyl}-3-fluoropyrrolidine-1-sulfonamide;
(3R)—N-[3-(1-{5-[4-({1-[4-(2,4-dioxo-1,3-diazinan-1-yl)phenyl]piperidin-4-yl}methyl)piperazin-1-yl]pyridin-2-yl}-3-(pyridin-4-yl)-1H-pyrazol-4-yl)-2,5-difluorophenyl]-3-fluoropyrrolidine-1-sulfonamide; (3R)—N-{3-[1-(5-{4-[(1-{5-[(3RS)-2,6-dioxopiperidin-3-yl]pyridin-2-yl}piperidin-4-yl)methyl]piperazin-1-yl}pyridin-2-yl)-3-(pyridin-4-yl)-1H-pyrazol-4-yl]-2,5-difluorophenyl}-3-fluoropyrrolidine-1-sulfonamide;
(3R)—N-{3-[1-(5-{4-[(1-{5-[(3R)-2,6-dioxopiperidin-3-yl]pyridin-2-yl}piperidin-4-yl)methyl]piperazin-1-yl}pyridin-2-yl)-3-(pyridin-4-yl)-1H-pyrazol-4-yl]-2,5-difluorophenyl}-3-fluoropyrrolidine-1-sulfonamide; (3R)-3-(4-{4-[(4-{4-[4-(5-chloro-3-{[ethyl(methyl)sulfamoyl]amino}-2-fluorophenyl)-3-(pyridin-4-yl)-1H-pyrazol-1-yl]phenyl}piperazin-1-yl)methyl]piperidin-1-yl}phenyl)piperidine-2,6-dione; 1-(4-{4-[(4-{4-[4-(5-chloro-3-{[ethyl(methyl)sulfamoyl]amino}-2-fluorophenyl)-3-(pyridin-4-yl)-1H-pyrazol-1-yl]phenyl}piperazin-1-yl)methyl]piperidin-1-yl}phenyl)-1,3-diazinane-2,4-dione;
rac-(3R)-3-(6-{4-[(4-{4-[4-(5-chloro-3-{[ethyl(methyl)sulfamoyl]amino}-2-fluorophenyl)-3-(pyridin-4-yl)-1H-pyrazol-1-yl]phenyl}piperazin-1-yl)methyl]piperidin-1-yl}pyridin-3-yl)piperidine-2,6-dione; (3R)-3-(6-{4-[(4-{4-[4-(5-chloro-3-{[ethyl(methyl)sulfamoyl]amino}-2-fluorophenyl)-3-(pyridin-4-yl)-1H-pyrazol-1-yl]phenyl}piperazin-1-yl)methyl]piperidin-1-yl}pyridin-3-yl)piperidine-2,6-dione; (3R)-3-(4-{4-[(4-{6-[4-(5-chloro-3-{[ethyl(methyl)sulfamoyl]amino}-2-fluorophenyl)-3-(pyridin-4-yl)-1H-pyrazol-1-yl]pyridin-3-yl}piperazin-1-yl)methyl]piperidin-1-yl}phenyl)piperidine-2,6-dione; 1-(4-{4-[(4-{6-[4-(5-chloro-3-{[ethyl(methyl)sulfamoyl]amino}-2-fluorophenyl)-3-(pyridin-4-yl)-1H-pyrazol-1-yl]pyridin-3-yl}piperazin-1-yl)methyl]piperidin-1-yl}phenyl)-1,3-diazinane-2,4-dione; rac-(3R)-3-(6-{4-[(4-{6-[4-(5-chloro-3-{[ethyl(methyl)sulfamoyl]amino}-2-fluorophenyl)-3-(pyridin-4-yl)-1H-pyrazol-1-yl]pyridin-3-yl}piperazin-1-yl)methyl]piperidin-1-yl}pyridin-3-yl)piperidine-2,6-dione; 1-(6-{4-[(4-{6-[4-(5-chloro-3-{[ethyl(methyl)sulfamoyl]amino}-2-fluorophenyl)-3-(pyridin-4-yl)-1H-pyrazol-1-yl]pyridin-3-yl}piperazin-1-yl)methyl]piperidin-1-yl}pyridin-3-yl)-1,3-diazinane-2,4-dione; (3R)-3-(6-{4-[(4-{6-[4-(5-chloro-3-{[ethyl(methyl)sulfamoyl]amino}-2-fluorophenyl)-3-(pyridin-4-yl)-1H-pyrazol-1-yl]pyridin-3-yl}piperazin-1-yl)methyl]piperidin-1-yl}pyridin-3-yl)piperidine-2,6-dione; (3R)—N-[5-chloro-3-(1-{4-[(3R)-4-({1-[4-(2,4-dioxo-1,3-diazinan-1-yl)phenyl]piperidin-4-yl}methyl)-3-methylpiperazin-1-yl]phenyl}-3-(pyridin-4-yl)-1H-pyrazol-4-yl)-2-fluorophenyl]-3-fluoropyrrolidine-1-sulfonamide; N-[5-chloro-3-(1-{4-[(3R)-4-[(1-{4-[(3R)-2,6-dioxopiperidin-3-yl]phenyl}piperidin-4-yl)methyl]-3-methylpiperazin-1-yl]phenyl}-3-(pyridin-4-yl)-1H-pyrazol-4-yl)-2-fluorophenyl]pyrrolidine-1-sulfonamide; (3R)—N-[5-chloro-3-(1-{4-[(3R)-4-[(1-{4-[(3R)-2,6-dioxopiperidin-3-yl]phenyl}piperidin-4-yl)methyl]-3-methylpiperazin-1-yl]phenyl}-3-(pyridin-4-yl)-1H-pyrazol-4-yl)-2-fluorophenyl]-3-fluoropyrrolidine-1-sulfonamide; (3R)—N-[5-chloro-3-(1-{4-[(3R)-4-[(1-{5-[(3RS)-2,6-dioxopiperidin-3-yl]pyridin-2-yl}piperidin-4-yl)methyl]-3-methylpiperazin-1-yl]phenyl}-3-(pyridin-4-yl)-1H-pyrazol-4-yl)-2-fluorophenyl]-3-fluoropyrrolidine-1-sulfonamide; N-[5-chloro-3-(1-{4-[(3R)-4-({1-[4-(2,4-dioxo-1,3-diazinan-1-yl)phenyl]piperidin-4-yl}methyl)-3-methylpiperazin-1-yl]phenyl}-3-(pyridin-4-yl)-1H-pyrazol-4-yl)-2-fluorophenyl]pyrrolidine-1-sulfonamide;
(3R)—N-[5-chloro-3-(1-{5-[(3R)-4-[(1-{4-[(3R)-2,6-dioxopiperidin-3-yl]phenyl}piperidin-4-yl)methyl]-3-methylpiperazin-1-yl]pyridin-2-yl}-3-(pyridin-4-yl)-1H-pyrazol-4-yl)-2-fluorophenyl]-3-fluoropyrrolidine-1-sulfonamide; (3R)—N-[5-chloro-3-(1-{5-[(3R)-4-[(1-{5-[(3RS)-2,6-dioxopiperidin-3-yl]pyridin-2-yl}piperidin-4-yl)methyl]-3-methylpiperazin-1-yl]pyridin-2-yl}-3-(pyridin-4-yl)-1H-pyrazol-4-yl)-2-fluorophenyl]-3-fluoropyrrolidine-1-sulfonamide; (3R)—N-[5-chloro-3-(1-{5-[(3R)-4-({1-[4-(2,4-dioxo-1,3-diazinan-1-yl)phenyl]piperidin-4-yl}methyl)-3-methylpiperazin-1-yl]pyridin-2-yl}-3-(pyridin-4-yl)-1H-pyrazol-4-yl)-2-fluorophenyl]-3-fluoropyrrolidine-1-sulfonamide; N-[5-chloro-3-(1-{5-[(3R)-4-[(1-{5-[(3RS)-2,6-dioxopiperidin-3-yl]pyridin-2-yl}piperidin-4-yl)methyl]-3-methylpiperazin-1-yl]pyridin-2-yl}-3-(pyridin-4-yl)-1H-pyrazol-4-yl)-2-fluorophenyl]pyrrolidine-1-sulfonamide; N-[5-chloro-3-(1-{5-[(3R)-4-({1-[4-(2,4-dioxo-1,3-diazinan-1-yl)phenyl]piperidin-4-yl}methyl)-3-methylpiperazin-1-yl]pyridin-2-yl}-3-(pyridin-4-yl)-1H-pyrazol-4-yl)-2-fluorophenyl]pyrrolidine-1-sulfonamide; N-[5-chloro-3-(1-{5-[(3R)-4-[(1-{5-[(3R)-2,6-dioxopiperidin-3-yl]pyridin-2-yl}piperidin-4-yl)methyl]-3-methylpiperazin-1-yl]pyridin-2-yl}-3-(pyridin-4-yl)-1H-pyrazol-4-yl)-2-fluorophenyl]pyrrolidine-1-sulfonamide; N-[5-chloro-3-(1-{5-[(3R)-4-[(1-{4-[(3R)-2,6-dioxopiperidin-3-yl]phenyl}piperidin-4-yl)methyl]-3-methylpiperazin-1-yl]pyridin-2-yl}-3-(pyridin-4-yl)-1H-pyrazol-4-yl)-2-fluorophenyl]pyrrolidine-1-sulfonamide; (RS)—N-(5-chloro-3-(1-(4-(4-((1-(5-((R)-2,6-dioxopiperidin-3-yl)pyridin-2-yl)piperidin-4-yl)methyl)piperazin-1-yl)phenyl)-3-(pyridin-4-yl)-1H-pyrazol-4-yl)-2-fluorophenyl)butane-2-sulfonamide; rac-(2R)—N-[5-chloro-3-(1-{4-[4-({1-[4-(2,4-dioxo-1,3-diazinan-1-yl)phenyl]piperidin-4-yl}methyl)piperazin-1-yl]phenyl}-3-(pyridin-4-yl)-1H-pyrazol-4-yl)-2-fluorophenyl]butane-2-sulfonamide; rac-(R)—N-(5-chloro-3-(1-(4-(4-((1-(5-((S)-2,6-dioxopiperidin-3-yl)pyridin-2-yl)piperidin-4-yl)methyl)piperazin-1-yl)phenyl)-3-(pyridin-4-yl)-1H-pyrazol-4-yl)-2-fluorophenyl)butane-2-sulfonamide; (2RS)—N-{5-chloro-3-[1-(4-{4-[(1-{4-[(3R)-2,6-dioxopiperidin-3-yl]phenyl}piperidin-4-yl)methyl]piperazin-1-yl}phenyl)-3-(pyridin-4-yl)-1H-pyrazol-4-yl]-2-fluorophenyl}butane-2-sulfonamide; N-{5-chloro-3-[1-(4-{4-[(1-{5-[(3R)-2,6-dioxopiperidin-3-yl]pyridin-2-yl}piperidin-4-yl)methyl]piperazin-1-yl}phenyl)-3-(pyridin-4-yl)-1H-pyrazol-4-yl]-2-fluorophenyl}-3-fluoroazetidine-1-sulfonamide; rac-N-{5-chloro-3-[1-(4-{4-[(1-{5-[(3R)-2,6-dioxopiperidin-3-yl]pyridin-2-yl}piperidin-4-yl)methyl]piperazin-1-yl}phenyl)-3-(pyridin-4-yl)-1H-pyrazol-4-yl]-2-fluorophenyl}-3-fluoroazetidine-1-sulfonamide; N-{5-chloro-3-[1-(4-{4-[(1-{4-[(3R)-2,6-dioxopiperidin-3-yl]phenyl}piperidin-4-yl)methyl]piperazin-1-yl}phenyl)-3-(pyridin-4-yl)-1H-pyrazol-4-yl]-2-fluorophenyl}-3-fluoroazetidine-1-sulfonamide; (3R)—N-{5-chloro-3-[1-(4-{4-[(1-{5-[(3R)-2,6-dioxopiperidin-3-yl]pyridin-2-yl}piperidin-4-yl)methyl]piperazin-1-yl}-2-fluorophenyl)-3-(pyridin-4-yl)-1H-pyrazol-4-yl]-2-fluorophenyl}-3-fluoropyrrolidine-1-sulfonamide; (3R)—N-{5-chloro-3-[1-(4-{4-[(1-{5-[(3RS)-2,6-dioxopiperidin-3-yl]pyridin-2-yl}piperidin-4-yl)methyl]piperazin-1-yl}-2-fluorophenyl)-3-(pyridin-4-yl)-1H-pyrazol-4-yl]-2-fluorophenyl}-3-fluoropyrrolidine-1-sulfonamide; (3R)—N-{5-chloro-3-[1-(4-{4-[(1-{4-[(3R)-2,6-dioxopiperidin-3-yl]phenyl}piperidin-4-yl)methyl]piperazin-1-yl}-2-fluorophenyl)-3-(pyridin-4-yl)-1H-pyrazol-4-yl]-2-fluorophenyl}-3-fluoropyrrolidine-1-sulfonamide; rac-N-{5-chloro-3-[1-(4-{4-[(1-{5-[(3R)-2,6-dioxopiperidin-3-yl]pyridin-2-yl}piperidin-4-yl)methyl]piperazin-1-yl}-2-fluorophenyl)-3-(pyridin-4-yl)-1H-pyrazol-4-yl]-2-fluorophenyl}-3-fluoroazetidine-1-sulfonamide; N-{5-chloro-3-[1-(4-{4-[(1-{4-[(3R)-2,6-dioxopiperidin-3-yl]phenyl}piperidin-4-yl)methyl]piperazin-1-yl}-2-fluorophenyl)-3-(pyridin-4-yl)-1H-pyrazol-4-yl]-2-fluorophenyl}-3-fluoroazetidine-1-sulfonamide; N-[5-chloro-3-(1-{4-[4-({1-[4-(2,4-dioxo-1,3-diazinan-1-yl)phenyl]piperidin-4-yl}methyl)piperazin-1-yl]-2-fluorophenyl}-3-(pyridin-4-yl)-1H-pyrazol-4-yl)-2-fluorophenyl]-3-fluoroazetidine-1-sulfonamide; N-{3-[1-(4-{4-[(1-{4-[(3R)-2,6-dioxopiperidin-3-yl]phenyl}piperidin-4-yl)methyl]piperazin-1-yl}-2-fluorophenyl)-3-(pyridin-4-yl)-1H-pyrazol-4-yl]-2,5-difluorophenyl}pyrrolidine-1-sulfonamide; rac-N-{3-[1-(4-{4-[(1-{5-[(3R)-2,6-dioxopiperidin-3-yl]pyridin-2-yl}piperidin-4-yl)methyl]piperazin-1-yl}-2-fluorophenyl)-3-(pyridin-4-yl)-1H-pyrazol-4-yl]-2,5-difluorophenyl}pyrrolidine-1-sulfonamide; N-{3-[1-(5-{4-[(1-{4-[(3R)-2,6-dioxopiperidin-3-yl]phenyl}piperidin-4-yl)methyl]piperazin-1-yl}pyrimidin-2-yl)-3-(pyridin-4-yl)-1H-pyrazol-4-yl]-2,5-difluorophenyl}pyrrolidine-1-sulfonamide; rac-N-{3-[1-(5-{4-[(1-{5-[(3R)-2,6-dioxopiperidin-3-yl]pyridin-2-yl}piperidin-4-yl)methyl]piperazin-1-yl}pyrimidin-2-yl)-3-(pyridin-4-yl)-1H-pyrazol-4-yl]-2,5-difluorophenyl}pyrrolidine-1-sulfonamide; N-{5-chloro-3-[1-(4-{4-[(1-{4-[(3R)-2,6-dioxopiperidin-3-yl]-3-fluorophenyl}piperidin-4-yl)methyl]piperazin-1-yl}phenyl)-3-(pyridin-4-yl)-1H-pyrazol-4-yl]-2-fluorophenyl}pyrrolidine-1-sulfonamide; rac-N-{5-chloro-3-[1-(4-{4-[(1-{5-[(3R)-2,6-dioxopiperidin-3-yl]pyridin-2-yl}piperidin-4-yl)methyl]-1,4-diazepan-1-yl}-2-fluorophenyl)-3-(pyridin-4-yl)-1H-pyrazol-4-yl]-2-fluorophenyl}pyrrolidine-1-sulfonamide; N-{5-chloro-3-[1-(4-{4-[(1-{4-[(3R)-2,6-dioxopiperidin-3-yl]phenyl}piperidin-4-yl)methyl]-1,4-diazepan-1-yl}-2-fluorophenyl)-3-(pyridin-4-yl)-1H-pyrazol-4-yl]-2-fluorophenyl}pyrrolidine-1-sulfonamide; rac-N-{5-chloro-3-[1-(4-{4-[(1-{4-[(3R)-2,6-dioxopiperidin-3-yl]-2-fluorophenyl}piperidin-4-yl)methyl]piperazin-1-yl}phenyl)-3-(pyridin-4-yl)-1H-pyrazol-4-yl]-2-fluorophenyl}pyrrolidine-1-sulfonamide; rac-N-{5-chloro-3-[1-(4-{4-[(1-{4-[(3R)-2,6-dioxopiperidin-3-yl]-3-fluorophenyl}piperidin-4-yl)methyl]piperazin-1-yl}phenyl)-3-(pyridin-4-yl)-1H-pyrazol-4-yl]-2-fluorophenyl}pyrrolidine-1-sulfonamide; N-[5-(difluoromethyl)-3-[1-(4-{4-[(1-{4-[(3R)-2,6-dioxopiperidin-3-yl]phenyl}piperidin-4-yl)methyl]piperazin-1-yl}phenyl)-3-(pyridin-4-yl)-1H-pyrazol-4-yl]-2-fluorophenyl]pyrrolidine-1-sulfonamide; rac-N-[5-(difluoromethyl)-3-[1-(4-{4-[(1-{5-[(3R)-2,6-dioxopiperidin-3-yl]pyridin-2-yl}piperidin-4-yl)methyl]piperazin-1-yl}phenyl)-3-(pyridin-4-yl)-1H-pyrazol-4-yl]-2-fluorophenyl]pyrrolidine-1-sulfonamide; N-{5-chloro-3-[1-(6-{4-[(1-{4-[(3R)-2,6-dioxopiperidin-3-yl]phenyl}piperidin-4-yl)methyl]piperazin-1-yl}pyridin-3-yl)-3-(pyridin-4-yl)-1H-pyrazol-4-yl]-2-fluorophenyl}pyrrolidine-1-sulfonamide; rac-N-{5-chloro-3-[1-(6-{4-[(1-{5-[(3R)-2,6-dioxopiperidin-3-yl]pyridin-2-yl}piperidin-4-yl)methyl]piperazin-1-yl}pyridin-3-yl)-3-(pyridin-4-yl)-1H-pyrazol-4-yl]-2-fluorophenyl}pyrrolidine-1-sulfonamide; N-{5-chloro-3-[1-(4-{4-[(1-{4-[(3R)-2,6-dioxopiperidin-3-yl]phenyl}piperidin-4-yl)methyl]piperazin-1-yl}-2,5-difluorophenyl)-3-(pyridin-4-yl)-1H-pyrazol-4-yl]-2-fluorophenyl}pyrrolidine-1-sulfonamide; rac-N-{5-chloro-3-[1-(4-{4-[(1-{5-[(3R)-2,6-dioxopiperidin-3-yl]pyridin-2-yl}piperidin-4-yl)methyl]piperazin-1-yl}-2,5-difluorophenyl)-3-(pyridin-4-yl)-1H-pyrazol-4-yl]-2-fluorophenyl}pyrrolidine-1-sulfonamide; N-[5-chloro-3-(1-{4-[4-({1-[5-(2,4-dioxo-1,3-diazinan-1-yl)pyridin-2-yl]piperidin-4-yl}methyl)piperazin-1-yl]phenyl}-3-(pyridin-4-yl)-1H-pyrazol-4-yl)-2-fluorophenyl]pyrrolidine-1-sulfonamide; N-[5-(difluoromethyl)-3-(1-{4-[4-({1-[4-(2,4-dioxo-1,3-diazinan-1-yl)phenyl]piperidin-4-yl}methyl)piperazin-1-yl]-2-fluorophenyl}-3-(pyridin-4-yl)-1H-pyrazol-4-yl)-2-fluorophenyl]pyrrolidine-1-sulfonamide;
rac-N-[5-(difluoromethyl)-3-[1-(4-{4-[(1-{5-[(3R)-2,6-dioxopiperidin-3-yl]pyridin-2-yl}piperidin-4-yl)methyl]piperazin-1-yl}-2-fluorophenyl)-3-(pyridin-4-yl)-1H-pyrazol-4-yl]-2-fluorophenyl]pyrrolidine-1-sulfonamide; N-[5-chloro-3-(1-{5-[4-({1-[4-(2,4-dioxo-1,3-diazinan-1-yl)phenyl]piperidin-4-yl}methyl)piperazin-1-yl]pyrimidin-2-yl}-3-(pyridin-4-yl)-1H-pyrazol-4-yl)-2-fluorophenyl]pyrrolidine-1-sulfonamide; rac-N-{5-chloro-3-[1-(5-{4-[(1-{5-[(3R)-2,6-dioxopiperidin-3-yl]pyridin-2-yl}piperidin-4-yl)methyl]piperazin-1-yl}pyrimidin-2-yl)-3-(pyridin-4-yl)-1H-pyrazol-4-yl]-2-fluorophenyl}pyrrolidine-1-sulfonamide; N-{5-chloro-3-[1-(5-{4-[(1-{4-[(3R)-2,6-dioxopiperidin-3-yl]phenyl}piperidin-4-yl)methyl]piperazin-1-yl}pyrimidin-2-yl)-3-(pyridin-4-yl)-1H-pyrazol-4-yl]-2-fluorophenyl}pyrrolidine-1-sulfonamide; N-[5-chloro-3-(1-{4-[4-({1-[4-(2,4-dioxo-1,3-diazinan-1-yl)phenyl]piperidin-4-yl}methyl)piperazin-1-yl]-3-fluorophenyl}-3-(pyridin-4-yl)-1H-pyrazol-4-yl)-2-fluorophenyl]pyrrolidine-1-sulfonamide; N-[5-(difluoromethyl)-3-[1-(4-{4-[(1-{4-[(3R)-2,6-dioxopiperidin-3-yl]phenyl}piperidin-4-yl)methyl]piperazin-1-yl}-2-fluorophenyl)-3-(pyridin-4-yl)-1H-pyrazol-4-yl]-2-fluorophenyl]pyrrolidine-1-sulfonamide;
rac-N-{5-chloro-3-[1-(4-{4-[(1-{5-[(3R)-2,6-dioxopiperidin-3-yl]pyridin-2-yl}piperidin-4-yl)methyl]piperazin-1-yl}-3-fluorophenyl)-3-(pyridin-4-yl)-1H-pyrazol-4-yl]-2-fluorophenyl}pyrrolidine-1-sulfonamide; N-{5-chloro-3-[1-(4-{4-[(1-{4-[(3R)-2,6-dioxopiperidin-3-yl]phenyl}piperidin-4-yl)methyl]piperazin-1-yl}-3-fluorophenyl)-3-(pyridin-4-yl)-1H-pyrazol-4-yl]-2-fluorophenyl}pyrrolidine-1-sulfonamide; (3R)—N-[5-chloro-3-(1-{4-[4-({1-[4-(2,4-dioxo-1,3-diazinan-1-yl)phenyl]piperidin-4-yl}methyl)piperazin-1-yl]-3-fluorophenyl}-3-(pyridin-4-yl)-1H-pyrazol-4-yl)-2-fluorophenyl]-3-fluoropyrrolidine-1-sulfonamide; (3R)—N-{5-chloro-3-[1-(4-{4-[(1-{5-[(3RS)-2,6-dioxopiperidin-3-yl]pyridin-2-yl}piperidin-4-yl)methyl]piperazin-1-yl}-3-fluorophenyl)-3-(pyridin-4-yl)-1H-pyrazol-4-yl]-2-fluorophenyl}-3-fluoropyrrolidine-1-sulfonamide; (3R)—N-{5-chloro-3-[1-(4-{4-[(1-{4-[(3R)-2,6-dioxopiperidin-3-yl]phenyl}piperidin-4-yl)methyl]piperazin-1-yl}-3-fluorophenyl)-3-(pyridin-4-yl)-1H-pyrazol-4-yl]-2-fluorophenyl}-3-fluoropyrrolidine-1-sulfonamide; N-[5-chloro-3-(1-{4-[4-({1-[5-(2,4-dioxo-1,3-diazinan-1-yl)pyridin-2-yl]piperidin-4-yl}methyl)piperazin-1-yl]phenyl}-3-(pyridin-4-yl)-1H-pyrazol-4-yl)-2-fluorophenyl]-3-fluoroazetidine-1-sulfonamide; N-[5-chloro-3-(1-{4-[4-({1-[5-(2,4-dioxo-1,3-diazinan-1-yl)pyridin-2-yl]piperidin-4-yl}methyl)piperazin-1-yl]-2-fluorophenyl}-3-(pyridin-4-yl)-1H-pyrazol-4-yl)-2-fluorophenyl]-3-fluoroazetidine-1-sulfonamide; N-[5-chloro-3-(1-{4-[4-({1-[5-(2,4-dioxo-1,3-diazinan-1-yl)pyridin-2-yl]piperidin-4-yl}methyl)piperazin-1-yl]-2-fluorophenyl}-3-(pyridin-4-yl)-1H-pyrazol-4-yl)-2-fluorophenyl]pyrrolidine-1-sulfonamide; (3R)—N-[5-chloro-3-(1-{4-[4-({1-[5-(2,4-dioxo-1,3-diazinan-1-yl)pyridin-2-yl]piperidin-4-yl}methyl)piperazin-1-yl]-2-fluorophenyl}-3-(pyridin-4-yl)-1H-pyrazol-4-yl)-2-fluorophenyl]-3-fluoropyrrolidine-1-sulfonamide; N-{5-chloro-3-[1-(4-{4-[(1-{5-[(3R)-2,6-dioxopiperidin-3-yl]pyridin-2-yl}piperidin-4-yl)methyl]piperazin-1-yl}phenyl)-3-(pyridin-4-yl)-1H-pyrazol-4-yl]-2-fluorophenyl}cyclopentanesulfonamide; rac-N-{5-chloro-3-[1-(4-{4-[(1-{5-[(3R)-2,6-dioxopiperidin-3-yl]pyridin-2-yl}piperidin-4-yl)methyl]piperazin-1-yl}phenyl)-3-(pyridin-4-yl)-1H-pyrazol-4-yl]-2-fluorophenyl}cyclopentanesulfonamide; N-{5-chloro-3-[1-(4-{4-[(1-{4-[(3R)-2,6-dioxopiperidin-3-yl]phenyl}piperidin-4-yl)methyl]piperazin-1-yl}phenyl)-3-(pyridin-4-yl)-1H-pyrazol-4-yl]-2-fluorophenyl}cyclopentanesulfonamide; (3R)-3-(6-{4-[(4-{4-[4-(3-{[ethyl(methyl)sulfamoyl]amino}-2,5-difluorophenyl)-3-(pyridin-4-yl)-1H-pyrazol-1-yl]phenyl}piperazin-1-yl)methyl]piperidin-1-yl}pyridin-3-yl)piperidine-2,6-dione; rac-(3R)-3-(6-{4-[(4-{4-[4-(3-{[ethyl(methyl)sulfamoyl]amino}-2,5-difluorophenyl)-3-(pyridin-4-yl)-1H-pyrazol-1-yl]phenyl}piperazin-1-yl)methyl]piperidin-1-yl}pyridin-3-yl)piperidine-2,6-dione; (3R)-3-(4-{4-[(4-{4-[4-(3-{[ethyl(methyl)sulfamoyl]amino}-2,5-difluorophenyl)-3-(pyridin-4-yl)-1H-pyrazol-1-yl]phenyl}piperazin-1-yl)methyl]piperidin-1-yl}phenyl)piperidine-2,6-dione; N-[3-(1-{4-[(3R)-4-[(1-{4-[(3R)-2,6-dioxopiperidin-3-yl]phenyl}piperidin-4-yl)methyl]-3-methylpiperazin-1-yl]phenyl}-3-(pyridin-4-yl)-1H-pyrazol-4-yl)-2-fluorophenyl]propane-1-sulfonamide; (3R)-3-(6-{4-[(4-{4-[4-(3-{[ethyl(methyl)sulfamoyl]amino}-2-fluorophenyl)-3-(pyridin-4-yl)-1H-pyrazol-1-yl]-3-fluorophenyl}piperazin-1-yl)methyl]piperidin-1-yl}pyridin-3-yl)piperidine-2,6-dione; (3S)-3-(6-{4-[(4-{4-[4-(3-{[ethyl(methyl)sulfamoyl]amino}-2-fluorophenyl)-3-(pyridin-4-yl)-1H-pyrazol-1-yl]-2-fluorophenyl}piperazin-1-yl)methyl]piperidin-1-yl}pyridin-3-yl)piperidine-2,6-dione; rac-(R)—N-(3-(1-(4-(4-((4-(4-(2,6-dioxopiperidin-3-yl)phenyl)piperazin-1-yl)methyl)piperidin-1-yl)phenyl)-3-(pyridin-4-yl)-1H-pyrazol-4-yl)-2-fluorophenyl)propane-1-sulfonamide; N-(5-chloro-3-(1-(4-((3R,5R)-4-((1-(5-((RS)-2,6-dioxopiperidin-3-yl)pyridin-2-yl)piperidin-4-yl)methyl)-3,5-dimethylpiperazin-1-yl)-2-fluorophenyl)-3-(pyridin-4-yl)-1H-pyrazol-4-yl)-2-fluorophenyl)pyrrolidine-1-sulfonamide; N-{5-chloro-3-[1-(5-{4-[(1-{5-[(3S)-2,6-dioxopiperidin-3-yl]pyridin-2-yl}piperidin-4-yl)methyl]piperazin-1-yl}pyridin-2-yl)-3-(pyridin-4-yl)-1H-pyrazol-4-yl]-2-fluorophenyl}pyrrolidine-1-sulfonamide; N-{5-chloro-3-[1-(4-{4-[(1-{4-[(3S)-2,6-dioxopiperidin-3-yl]-3-fluorophenyl}piperidin-4-yl)methyl]piperazin-1-yl}phenyl)-3-(pyridin-4-yl)-1H-pyrazol-4-yl]-2-fluorophenyl}pyrrolidine-1-sulfonamide; rac-N-{5-chloro-3-[1-(4-{4-[(1-{4-[(3R)-2,6-dioxopiperidin-3-yl]-2,3-difluorophenyl}piperidin-4-yl)methyl]piperazin-1-yl}phenyl)-3-(pyridin-4-yl)-1H-pyrazol-4-yl]-2-fluorophenyl}pyrrolidine-1-sulfonamide; rac-N-{5-chloro-3-[1-(4-{4-[(1-{4-[(3R)-2,6-dioxopiperidin-3-yl]-2,5-difluorophenyl}piperidin-4-yl)methyl]piperazin-1-yl}phenyl)-3-(pyridin-4-yl)-1H-pyrazol-4-yl]-2-fluorophenyl}pyrrolidine-1-sulfonamide; rac-N-{5-chloro-3-[1-(4-{4-[(1-{4-[(3R)-2,6-dioxopiperidin-3-yl]-3,5-difluorophenyl}piperidin-4-yl)methyl]piperazin-1-yl}phenyl)-3-(pyridin-4-yl)-1H-pyrazol-4-yl]-2-fluorophenyl}pyrrolidine-1-sulfonamide; and N-[5-chloro-3-(1-{4-[4-({1-[5-(2,4-dioxo-1,3-diazinan-1-yl)pyridin-2-yl]piperidin-4-yl}methyl)piperazin-1-yl]phenyl}-3-(pyridin-4-yl)-1H-pyrazol-4-yl)-2-fluorophenyl]cyclopentanesulfonamide.

84. A pharmaceutical composition comprising the compound of any one of the previous claims and a pharmaceutically acceptable carrier, excipient, and/or diluent.

85. A method of treating a disease or disorder in a subject in need thereof comprising administering to the subject a therapeutically effective amount of the compound or composition of any one of the previous claims.

86. The method of claim 85, wherein the disease or disorder is cancer.

87. The compound or composition of any one of the previous claims for use in therapy.

88. The compound or composition of any one of the previous claims for use in the treatment of cancer.
